(19) United States
(12) Patent Application Publication
(10) Pub. No.: US 2025/0177374 A1
(43) Pub. Date: Jun. 5, 2025

(54) BCL-XL Inhibitory Compounds Having Low Cell Permeability and Antibody Drug Conjugates Including the Same

(71) Applicant: AbbVie Inc., North Chicago, IL (US)

(72) Inventors: Scott L. Ackler, Gurnee, IL (US); Nathan B. Bennett, Gurnee, IL (US); Erwin R. Boghaert, Pleasant Prairie, WI (US); Steve C. Cullen, Lake Villa, IL (US); George Doherty, Libertyville, IL (US); Robin R. Frey, Libertyville, IL (US); Anthony R. Haight, Wadsworth, IL (US); Andrew S. Judd, Grayslake, IL (US); Aaron R. Kunzer, Arlington Heights, IL (US); Violeta L. Marin, Chicago, IL (US); Xiaoqiang Shen, Lincolnshire, IL (US); Xiaohong Song, Grayslake, IL (US); Andrew J. Souers, Libertyville, IL (US); Gerard M. Sullivan, Lake Villa, IL (US); Zhi-Fu Tao, Vernon Hills, IL (US); Xilu Wang, Libertyville, IL (US); Dennie S. Welch, Gurnee, IL (US); Michael D. Wendt, Vernon Hills, IL (US)

(21) Appl. No.: 18/766,249

(22) Filed: Jul. 8, 2024

Related U.S. Application Data
(63) Continuation of application No. 17/804,696, filed on May 31, 2022, now abandoned.
Application No. 17/804,696 is a continuation of application No. 16/563,507, filed on Sep. 6, 2019, now abandoned.
Application No. 16/563,507 is a continuation of application No. 16/240,544, filed on Jan. 4, 2019, now abandoned.
Application No. 16/240,544 is a continuation of application No. 14/963,506, filed on Dec. 9, 2015, now abandoned.
(60) Provisional application No. 62/089,780, filed on Dec. 9, 2014.

(51) Int. Cl. A61K 31/4709; A61K 31/4725; A61K 31/498; A61K 31/4985; A61K 31/538; A61K 45/06; A61K 47/68; C07D 417/14; C07D 487/04; C07D 513/04

(52) U.S. Cl. CPC A61K 31/4709; A61K 31/4725; A61K 31/498; A61K 31/4985; A61K 31/538; A61K 45/06; A61K 47/6803; A61K 47/6849; A61K 47/6851; A61K 47/6883; A61K 47/6889; C07D 417/14; C07D 487/04; C07D 513/04

(57) ABSTRACT

The present disclosure concerns Bcl-xL inhibitors having low cell permeability, antibody drug conjugates (ADCs) comprising the inhibitors, synthons useful for synthesizing the ADCs, compositions comprising the inhibitors or ADCs, and various methods of using the inhibitors and ADCs.

### 1. FIELD

The present disclosure pertains to compounds that inhibit the activity of Bcl-xL anti-apoptotic proteins, antibody drug conjugates comprising these inhibitors, methods useful for synthesizing these inhibitors and antibody drug conjugates, compositions comprising the inhibitors, and antibody drug conjugates, and methods of treating diseases in which anti-apoptotic Bcl-xL proteins are expressed.

### 2. BACKGROUND

Apoptosis is recognized as an essential biological process for tissue homeostasis of all living species. In mammals in particular, it has been shown to regulate early embryonic development. Later in life, cell death is a default mechanism by which potentially dangerous cells (e.g., cells carrying cancerous defects) are removed. Several apoptotic pathways have been uncovered, and one of the most important involves the Bcl-2 family of proteins, which are key regulators of the mitochondrial (also called “intrinsic”) pathway of apoptosis. See, Danial & Korsmeyer, 2004, Cell 116:205-219.

Dysregulated apoptotic pathways have been implicated in the pathology of many significant diseases such as neurodegenerative conditions (up-regulated apoptosis), such as for example, Alzheimer's disease; and proliferative diseases (down-regulated apoptosis) such as for example, cancer, autoimmune diseases and pro-thrombotic conditions.

In one aspect, the implication that down-regulated apoptosis (and more particularly the Bcl-2 family of proteins) is involved in the onset of cancerous malignancy has revealed a novel way of targeting this still elusive disease. Research has shown, for example, the anti-apoptotic proteins, Bcl-2 and Bcl-xL, are over-expressed in many cancer cell types. See, Zhang, 2002, Nature Reviews/Drug Discovery 1:101; Kirkin et al., 2004, Biochimica Biophysica Acta 1644:229-249; and Amundson et al., 2000, Cancer Research 60:6101-6110. The effect of this deregulation is the survival of altered cells which would otherwise have undergone apoptosis in normal conditions. The repetition of these defects associated with unregulated proliferation is thought to be the starting point of cancerous evolution.

These findings as well as numerous others have made possible the emergence of new strategies in drug discovery for targeting cancer. If a small molecule were able to enter the cell and overcome the anti-apoptotic protein over-expression, then it could be possible to reset the apoptotic process. This strategy can have the advantage that it can alleviate the problem of drug resistance which is usually a consequence of apoptotic deregulation (abnormal survival).

Researchers also have demonstrated that platelets also contain the necessary apoptotic machinery (e.g., Bax, Bak, Bcl-xL, Bcl-2, cytochrome c, caspase-9, caspase-3 and APAF-1) to execute programmed cell death through the intrinsic apoptotic pathway. Although circulating platelet production is a normal physiological process, a number of diseases are caused or exacerbated by excess of, or undesired activation of, platelets. The above suggests that therapeutic agents capable of inhibiting anti-apoptotic proteins in platelets and reducing the number of platelets in mammals may be useful in treating pro-thrombotic conditions and diseases that are characterized by an excess of, or undesired activation of, platelets.

Numerous Bcl-xL inhibitors have been developed for treatment of diseases (e.g., cancer) that involve dysregulated apoptotic pathways. However, Bcl-xL inhibitors can act on cells other than the target cells (e.g., cancer cells). For instance, pre-clinical studies have shown that pharmacological inactivation of Bcl-xL reduces platelet half-life and causes thrombocytopenia (see Mason et al., 2007, Cell 128:1173-1186).

Given the importance of Bcl-xL in regulating apoptosis, there remains a need in the art for agents that inhibit Bcl-xL activity, either selectively or non-selectively, as an approach towards the treatment of diseases in which apoptosis is dysregulated via expression or over-expression of anti-apoptotic Bcl-2 family proteins, such as Bcl-xL. Accordingly, new Bcl-xL inhibitors with reduced dose-limiting toxicity are needed.

Additionally, new methods of delivering Bcl-xL inhibitors that limit toxicity are needed. One potential means of delivering a drug to a cell which has not been explored for Bcl-xL inhibitors is delivery through the use of antibody drug conjugates (ADCs). ADCs are formed by chemically linking a cytotoxic drug to a monoclonal antibody through a linker. The monoclonal antibody of an ADC selectively binds to a target antigen of a cell (e.g., cancer cell) and releases the drug into the cell. ADCs have therapeutic potential because they combine the specificity of the antibody and the cytotoxic potential of the drug. Nonetheless, developing ADCs as therapeutic agents has thus far met with limited success owing to a variety of factors such as unfavorable toxicity profiles, low efficacies and poor pharmacological parameters. Accordingly, the development of new ADCs that overcome these problems and can selectively deliver Bcl-xL to target cancer cells would be a significant discovery.

### 3. SUMMARY

It has now been discovered that small molecule inhibitors of Bcl-xL are efficacious when administered in the form of antibody drug conjugates (ADCs; also called immunoconjugates) that bind to antigens expressed on the surface of cells where inhibition of Bcl-xL and consequent induction of apoptosis would be beneficial. This discovery provides, for the first time, the ability to target Bcl-xL inhibitory therapies to specific cells and/or tissues of interest, potentially lowering serum levels necessary to achieve desired therapeutic benefit and/or avoiding and/or ameliorating potential side effects associated with systemic administration of the small molecule Bcl-xL inhibitors per se.

Accordingly, in one aspect, the present disclosure provides ADCs comprising Bcl-xL inhibitors useful for, among other things, inhibiting anti-apoptotic Bcl-xL proteins as a therapeutic approach towards the treatment of diseases that involve a dysregulated apoptosis pathway (e.g., cancer). The ADCs generally comprise small molecule inhibitors of Bcl-xL (referred to herein as Bcl-xL inhibitors) linked by way of linkers to an antibody that specifically binds an antigen expressed on a target cell of interest.

In one aspect, the disclosure provides Bcl-xL inhibitors that have low cell-permeability. The Bcl-xL inhibitors may be used therapeutically as a component of an ADC or may be used independently from the ADCs. The Bcl-xL inhibitors described herein include solubilizing hydrophilic groups that increase water solubility and decrease the cell permeability as compared to similar inhibitors without the solubilizing groups. In certain embodiments, solubilizing group comprises a moiety capable of hydrogen bonding, dipole-dipole interactions, and/or that contains a polyol, a polyethylene glycol polymeric moiety, a salt or a moiety that is charged at physiological pH. In certain embodiments, the Bcl-xL inhibitors of the disclosure have very low cell permeability.

In embodiments where the Bcl-xL inhibitor is a component of an ADC, the use of a low cell-permeable Bcl-xL inhibitor can have benefits in that, once released from the antibody within a cell, it will have limited ability to permeate other cells and cause effects other than the intended anti-tumor effect. For instance, following internalization by ADC delivery, the Bcl-xL inhibitors of the disclosure are less likely to diffuse out of the cell than cell-permeable inhibitors, likely decreasing or ameliorating any undesirable side effects associated with systemic levels of the compound. Likewise, if Bcl-xL inhibitors of the disclosure are released into the systemic circulation prior to the antibody of the ADC binding to its target antigen, the released Bcl-xL inhibitors would diffuse into healthy cells much slower than the inhibitors without solubilizing groups, which may also result in reduced toxicity.

In addition to reduced toxicity, the low cell-permeable Bcl-xL inhibitors of the disclosure confer other beneficial properties to the ADCs. For instance, inclusion of a charged moiety on the Bcl-xL inhibitors increases water solubility of the ADCs and modulates the physiochemical properties of the ADCs. Furthermore, ADCs of the disclosure have much less of a tendency to aggregate that ADCs derived from Bcl-xL inhibitors that do not contain solubilizing groups. As a result, the Bcl-xL inhibitors of the disclosure are compatible with a larger array of linkers that link the antibody of the ADC with the inhibitor as compared to Bcl-xL inhibitors without solubilizing groups.

The antibody of an ADC may be any antibody that binds, typically but not necessarily specifically, to an antigen expressed on the surface of a target cell of interest. Target cells of interest will generally include cells where induction of apoptosis via inhibition of anti-apoptotic Bcl-xL proteins is desirable, including, by way of example and not limitation, tumor cells that express or over-express Bcl-xL. Target antigens may be any protein, glycoprotein, etc. expressed on the target cell of interest, but will typically be proteins or glycoproteins that are either uniquely expressed on the target cell and not on normal or healthy cells, or that are over-expressed on the target cell as compared to normal or healthy cells, such that the ADCs selectively target specific cells of interest, such as, for example, tumor cells. As is well-known in the art, ADCs bound to certain cell-surface antigens that internalize a bound ADC have certain advantages. Accordingly, in some embodiments, the antigen targeted by the antibody is an antigen that has the ability to internalize an ADC bound thereto into the cell. However, the antigen targeted by the ADC need not be one that internalizes the bound ADC. Bcl-xL inhibitors released outside the target cell or tissue may enter the cell via passive diffusion or other mechanisms to inhibit Bcl-xL.

As will be appreciated by skilled artisans, the specific antigen, and hence antibody, selected will depend upon the identity of the desired target cell of interest. In certain specific therapeutic embodiments, the target antigen for the antibody of the ADC is an antigen that is not expressed on a normal or healthy cell type known or suspected of being dependent, at least in part, on Bcl-xL for survival. In other certain specific therapeutic embodiments, the antibody of the ADC is an antibody suitable for administration to humans.

A vast array of cell-specific antigens useful as therapeutic targets, as well as antibodies that bind these antigens, are known in the art, as are techniques for obtaining additional antibodies suitable for targeting known cell-specific antigens or later-discovered cell-specific antigens. Any of these various different antibodies may be included in the ADCs described herein.

The linkers linking the Bcl-xL inhibitors to the antibody of an ADC may be long, short, flexible, rigid, hydrophobic or hydrophilic in nature, or may comprise segments have different characteristics, such as segments of flexibility, segments of rigidity, etc. The linker may be chemically stable to extracellular environments, for example, chemically stable in the blood stream, or may include linkages that are not stable and release the Bcl-xL inhibitor in the extracellular millieu. In some embodiments, the linker includes linkages that are designed to release the Bcl-xL inhibitor upon internalization of the ADC within the cell. In some specific embodiments, the linker includes linkages designed to cleave and/or immolate or otherwise breakdown specifically or non-specifically inside cells. A wide variety of linkers useful for linking drugs to antibodies in the context of ADCs are known in the art. Any of these linkers, as well as other linkers, may be used to link the Bcl-xL inhibitors to the antibody of the ADCs described herein.

The number of Bcl-xL inhibitors linked to the antibody of an ADC can vary (called the “drug-to-antibody ratio,” or “DAR”), and will be limited only by the number of available attachments sites on the antibody and the number of inhibitors linked to a single linker. Typically, a linker will link a single Bcl-xL inhibitor to the antibody of an ADC. As long as the ADC does not exhibit unacceptable levels of aggregation under the conditions of use and/or storage, ADCs with DARs of twenty, or even higher, are contemplated. In some embodiments, the ADCs described herein may have a DAR in the range of about 1-10, 1-8, 1-6, or 14. In certain specific embodiments, the ADCs may have a DAR of 2, 3 or 4. In some embodiments, Bcl-xL inhibitors, linkers and DAR combinations are selected such that the resultant ADC does not aggregate excessively under conditions of use and/or storage.

The low permeable Bcl-xL inhibitors described herein are generally compounds according to the following structural formula (IIa), (IIb), (IIc) or (IId), below, and/or pharmaceutically acceptable salts thereof, where the various substituents Ar¹, Ar², Z¹, Z^{2a}, Z^{2b}, R′, R¹, R², R⁴, R^{11a}, R^{11b}, R¹² and R¹³ are as defined in the Detailed Description section:

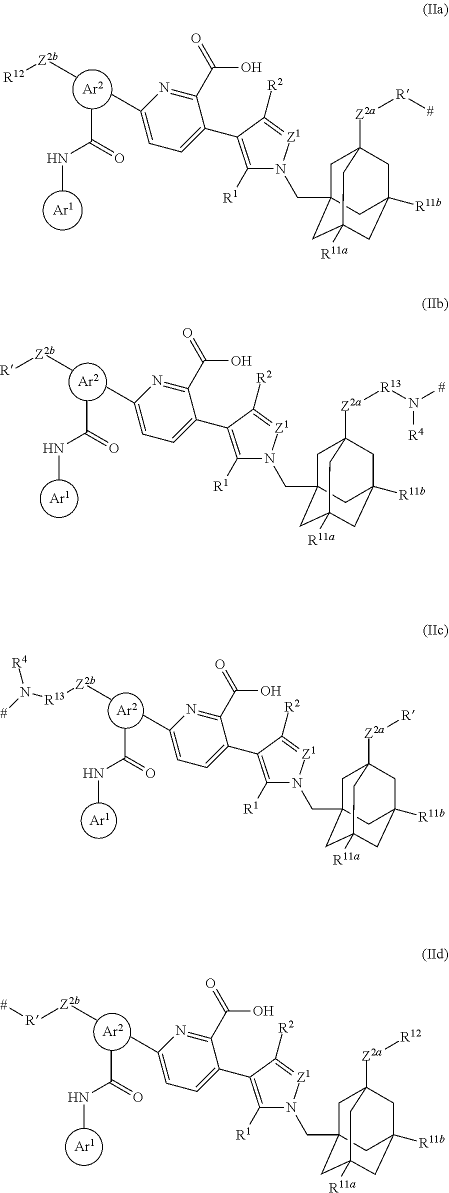

In formulae (IIa), (IIb), (IIc), (IId), #represents the point of attachment to the linker of an ADC or, for an inhibitor that is not part of an ADC, #represents a hydrogen atom.

In certain embodiments in which the Bcl-xL inhibitor is a compound of formula (IIa), the compound has the structural formula (IIa.1), below and or pharmaceutically acceptable salts thereof, where the various substituents Ar¹, Ar², Z¹, Z^{2a}, Z^{2b}, R¹, R², R^{11a}, R^{11b}, R¹², G, Y, r and s are as defined in the Detailed Description section:

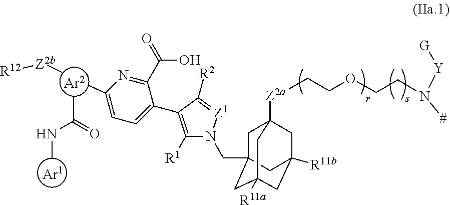

In certain embodiments in which the Bcl-xL inhibitor is a compound of formula (IIa), the compound has the structural formula (IIa.2), below and or pharmaceutically acceptable salts thereof, where the various substituents Ar¹, Ar², Z¹, Z^{2a}, Z^{2b}, R¹, R², R^{11a}, R^{11b}, R¹², U, V^{a}, V^{b}, R²⁰, R^{21a}, R^{21b} and s are as defined in the Detailed Description section:

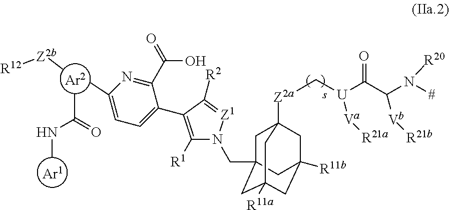

In certain embodiments in which the Bcl-xL inhibitor is a compound of formula (IIa), the compound has the structural formula (IIa.3), below and or pharmaceutically acceptable salts thereof, where the various substituents Ar¹, Ar², Z¹, Z^{2a}, Z^{2b}, R¹, R², R^{11a}, R^{11b}, R¹², G, J^{a}, T, R^{b} and s are as defined in the Detailed Description section:

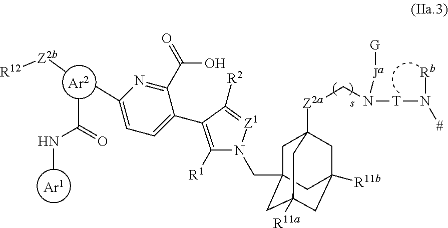

In certain embodiments in which the Bcl-xL inhibitor is a compound of formula (IIb), the compound has the structural formula (IIb.1), below and or pharmaceutically acceptable salts thereof, where the various substituents Ar¹, Ar², Z¹, Z^{2a}, Z^{2b}, G· R¹, R², R⁴, R^{11a}, R^{11b}, Y, r and s are as defined in the Detailed Description section:

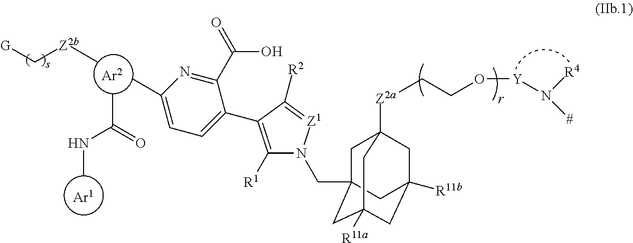

In certain embodiments in which the Bcl-xL inhibitor is a compound of formula (IIc), the compound has the structural formula (IIc.1), below and or pharmaceutically acceptable salts thereof, where the various substituents Ar¹, Ar², Z¹, Z^{2a}, Z^{2b}, G· R¹, R², R⁴, R^{11a}, R^{11b}, R²³, Y^{a} and Y^{b} are as defined in the Detailed Description section:

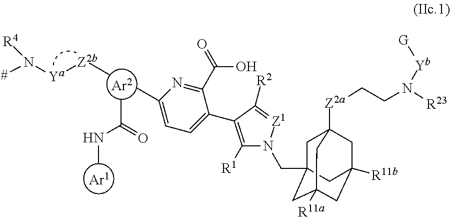

In certain embodiments in which the Bcl-xL inhibitor is a compound of formula (IIc), the compound has the structural formula (IIc.2), below and or pharmaceutically acceptable salts thereof, where the various substituents Ar¹, Ar², Z¹, Z^{2a}, Z^{2b}, G· R¹, R², R⁴, R^{11a}, R^{11b}, R²³, R²⁵, Y^{a}, Y^{b} and Y^{c} are as defined in the Detailed Description section:

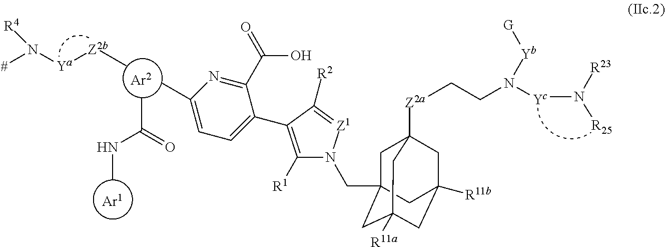

In certain embodiments in which the Bcl-xL inhibitor is a compound of formula (IId), the compound has the structural formula (IId.1), below and or pharmaceutically acceptable salts thereof, where the various substituents Ar¹, Ar², Z, Z², Z^{2b}, G, R¹, R², R^{11a}, R^{11b}, R²³, Y^{a}, Y^{b} and s are as defined in the Detailed Description section:

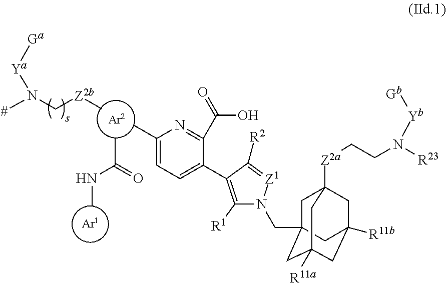

In some embodiments, the ADCs described herein are generally compounds according to structural formula (I):

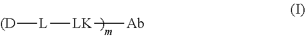

where Ab represents the antibody, D represents the drug (here, a Bcl-xL inhibitor), L represents the linker linking the drug D to the antibody Ab, LK represents a linkage formed between a functional group on linker L and a complementary functional group on antibody Ab, and m represents the number of linker-drug units linked to the antibody. In certain embodiments, Ab represents the antibody, D represents the drug, L represents the linker linking the drug D to the antibody Ab, LK represents a linkage formed between a functional group on linker L and a complementary functional group on antibody Ab, and m is 1 to 8. In certain embodiments, m is 1 to 20. In certain embodiments, m is 1 to 8. In certain embodiments, m is 2 to 8. In certain embodiments, m is 1 to 6. In certain embodiments, m is 2, 3, or 4.

In certain specific embodiments, the ADCs are compounds according to structural formula (Ia), (Ib), (Ic) and (Id), below, where the various substituents Ar¹, Ar², Z¹, Z^{2a}, Z^{2b}, R′, R¹, R², R^{11a}, R^{11b}, R¹² and R¹³ are as previously defined for formula (IIa), (IIb), (IIc), and (IId), respectively, Ab and L are as defined for structural formulae (I), LK represents a linkage formed between a functional group on linker L and a complementary functional group on antibody Ab, and m is an integer ranging from 1 to 20, and in some embodiments from 2 to 8:

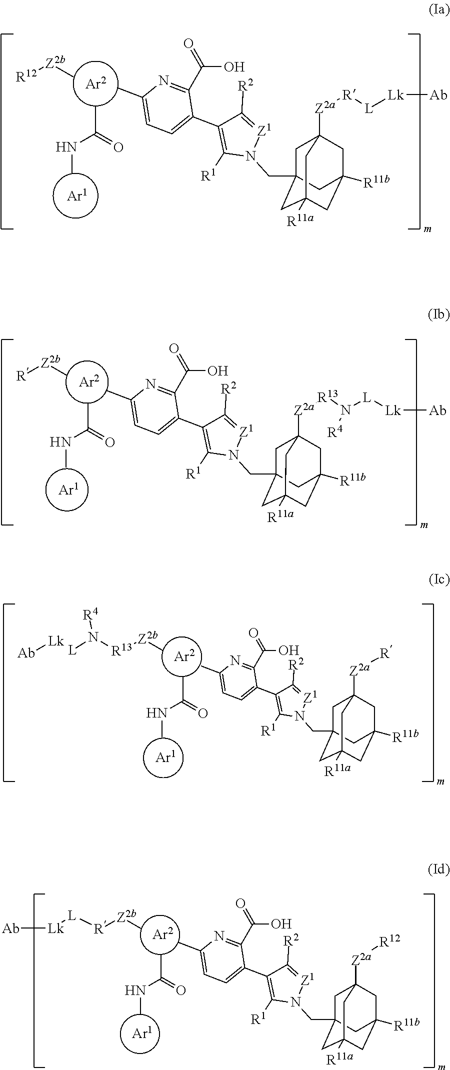

In another aspect, the present disclosure provides intermediate synthons useful for synthesizing the ADCs described herein, as well as methods for synthesizing the ADCs. The intermediate synthons generally comprise Bcl-xL inhibitors linked to a linker moiety that includes a functional group capable of linking the synthon to an antibody. The synthons are generally compounds according to structural formula (III), below, or salts thereof, where D is a Bcl-xL inhibitor as previously described herein, L is a linker as previously described and R^{x} comprises a functional group capable of conjugating the synthon to a complementary functional group on an antibody:

D-L-R^{x} (III)

In certain specific embodiments, the intermediate synthons are compounds according to structural formulae (IIIa), (IIIb), (IIIc) and (IIId), below, or salts thereof, where the various substituents Ar¹, Ar², Z¹, Z^{2a}, Z^{2b}, R′, R¹, R², R⁴, R^{11a}, R^{11b}, R¹² and R¹³ are as previously defined for structural formulae (ha), (IIb), (IIc) and (IId), respectively, L is a linker as previously described and R^{x} is a functional group as described above:

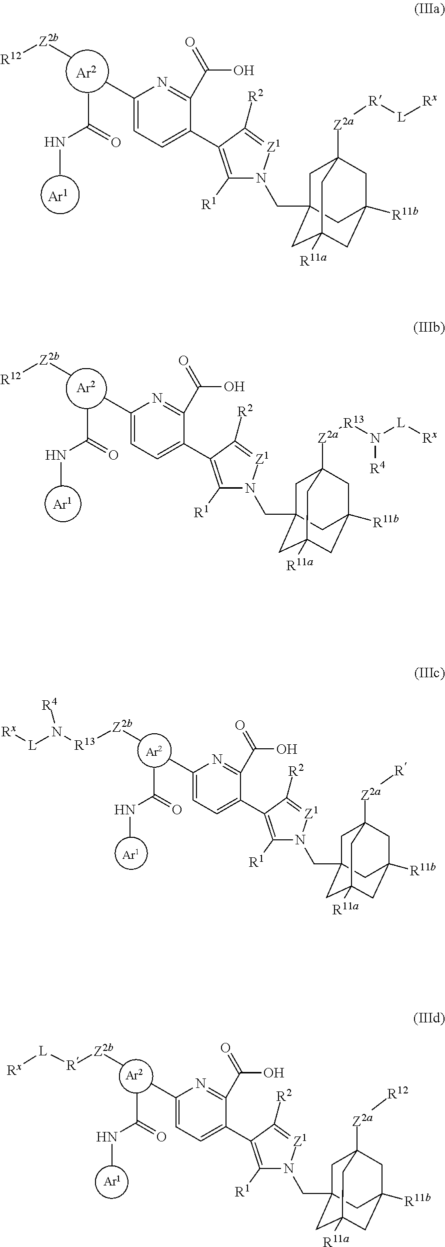

To synthesize an ADC, intermediate synthons according to structural formulae (III) or (IIIa)-(IIId), or salts thereof, are contacted with an antibody of interest under conditions in which functional group R^{x} reacts with a complementary functional group on the antibody to form a covalent linkage. The identity of group R^{x} will depend upon the desired coupling chemistry and the complementary groups on the antibody to which the synthons will be attached. Numerous groups suitable for conjugating molecules to antibodies are known in the art. Any of these groups may be suitable for R^{x}. Non-limiting exemplary functional groups (R^{x}) include NHS-esters, maleimides, haloacetyls, isothiocyanates, vinyl sulfones and vinyl sulfonamides. In certain embodiments, R^{x} comprises a functional group selected from the group consisting of NHS-esters, maleimides, haloacetyls, and isothiocyanates.

In another aspect, the present disclosure provides compositions including the Bcl-xL inhibitors or ADCs described herein. The compositions generally comprise one or more Bcl-xL inhibitors or ADCs as described herein, and/or salts thereof, and one or more excipients, carriers or diluents. The compositions may be formulated for pharmaceutical use, or other uses. In a specific embodiment, the composition is formulated for pharmaceutical use and comprises a Bcl-xL inhibitor according to structural formula (IIa), (IIb), (IIc) or (IId), or a pharmaceutically acceptable salt thereof, where #is hydrogen. In another embodiment, the composition is formulated for pharmaceutical use and comprises an ADC according to structural formula (Ia), (Ib), (Ic) or (IIId), or a pharmaceutically acceptable salt thereof, and one or more pharmaceutically acceptable excipients, carriers or diluents.

Bcl-xL inhibitory compositions formulated for pharmaceutical use may be packaged in bulk form suitable for multiple administrations, or may be packaged in the term of unit doses, such as for example tablets or capsules, suitable for a single administration. Likewise, ADC compositions formulated for pharmaceutical use may be packaged in bulk form suitable for multiple administrations, or may be packaged in the form of unit doses suitable for a single administration. Whether packaged in bulk or in the form of unit doses, the ADC composition may be a dry composition, such as a lyophilate, or a liquid composition. Unit dosage liquid ADC compositions may be conveniently packaged in the form of syringes pre-filled with an amount of ADC suitable for a single administration.

In still another aspect, the present disclosure provides methods of inhibiting anti-apoptotic Bcl-xL proteins. The method generally involves contacting an ADC as described herein, for example, an ADC according to structural formula (Ia), (Ib), (Ic) or (Id), or a salt thereof, with a target cell that expresses or overexpresses Bcl-xL and an antigen for the antibody of the ADC under conditions in which the antibody binds the antigen on the target cell. Depending upon the antigen, the ADC may become internalized into the target cell. The method may be carried out in vitro in a cellular assay to inhibit Bcl-xL activity, or in vivo as a therapeutic approach towards the treatment of diseases in which inhibition of Bcl-xL activity is desirable. The method may alternatively involve contacting a cell that expresses or over-expresses Bcl-xL with a Bcl-xL inhibitor, such as an inhibitor according to structural formula (IIa), (IIb), (IIc) or (IId), where #is hydrogen, or a salt thereof.

In still another aspect, the present disclosure provides methods of inducing apoptosis in cells. The method generally involves contacting an ADC as described herein, for example, an ADC according to structural formula (Ia), (Ib), (Ic) or (Id), or a salt thereof, with a target cell that expresses or overexpresses Bcl-xL and an antigen for the antibody of the ADC under conditions in which the antibody binds the antigen on the target cell. Depending upon the antigen, the ADC may become internalized into the target cell. The method may be carried out in vitro in a cellular assay to induce apoptosis, or in vivo as a therapeutic approach towards the treatment of diseases in which induction of apoptosis in specific cells would be beneficial. The method may alternatively involve contacting a cell that expresses or over-expresses Bcl-xL with a Bcl-xL inhibitor, for example an inhibitor according to structural formula (IIa), (IIb), (IIc) or (IId), where #is hydrogen, or a salt thereof.

In yet another aspect, the present disclosure provides methods of treating disease in which inhibition of Bcl-xL and/or induction of apoptosis would be desirable. As will be discussed more thoroughly in the Detailed Description section, a wide variety of diseases are mediated, at least in part, by dysregulated apoptosis stemming, at least in part, by expression or over-expression of anti-apoptotic Bcl-xL proteins. Any of these diseases may be treated or ameliorated with the Bcl-xL inhibitors or ADCs described herein.

The methods include administering to a subject suffering from a disease mediated, at least in part by expression or over-expression of Bcl-xL, an amount of a Bcl-xL inhibitor or ADC described herein effective to provide therapeutic benefit. For ADCs, the identity of the antibody of the ADC administered will depend upon the disease being treated. The therapeutic benefit achieved with the Bcl-xL inhibitors and ADCs described herein will also depend upon the disease being treated. In certain instances, the Bcl-xL inhibitory or ADC may treat or ameliorate the specific disease when administered as monotherapy. In other instances, the Bcl-xL inhibitor or ADC may be part of an overall treatment regimen including other agents that, together with the Bcl-xL inhibitor or ADC treat or ameliorate the disease.

For example, elevated expression levels of Bcl-xL have been associated with resistance to chemotherapy and radiation therapy in cancers. (Datta et al., 1995, Cell Growth Differ 6:363-370; Amundson et al., 2000, Cancer Res 60:6101-6110; Haura et al., 2004, Clin Lung Cancer 6:113-122). In the context of treating cancers, data disclosed herein establish that ADCs may be effective as monotherapy or may be effective when administered adjunctive to, or with, other targeted or non-targeted chemotherapeutic agents and/or radiation therapy. While not intending to be bound by any theory of operation, it is believed that inhibition of Bcl-xL activity with the Bcl-xL inhibitors and ADCs described herein in tumors that have become resistant to targeted or non-targeted chemo- and/or radiation therapies will “sensitize” the tumors such that they are again susceptible to the chemotherapeutic agents and/or radiation treatment. Certain embodiments pertain to a method of sensitizing a tumor to standard cytotoxic agents and/or radiation, comprising contacting the tumor with an ADC that is capable of binding the tumor, in an amount effective to sensitize the tumor cell to a standard cytotoxic agent and/or radiation. Another embodiment pertains to a method of sensitizing a tumor to standard cytotoxic agents and/or radiation, comprising contacting the tumor with an ADC that is capable of binding the tumor, in an amount effective to sensitize the tumor cell to a standard cytotoxic agent and/or radiation in which the tumor has become resistant to treatment with standard cytotoxic agents and/or radiation. Another embodiment pertains to a method of sensitizing a tumor to standard cytotoxic agents and/or radiation, comprising contacting the tumor with an ADC that is capable of binding the tumor, in an amount effective to sensitize the tumor cell to a standard cytotoxic agent and/or radiation in which the tumor has not been previously exposed to standard cytotoxic agents and/or radiation therapy.

Accordingly, in the context of treating cancers, “therapeutic benefit” includes administration of the Bcl-xL inhibitors and ADCs described herein adjunctive to, or with, targeted or non-targeted chemotherapeutic agents and/or radiation therapy, either in patients that have not yet begun the chemo- and/or radiation therapeutic regimens, or in patients that have exhibited resistance (or are suspected or becoming resistant) to the chemo- and/or radiation therapeutic regimens, as a means of sensitizing the tumors to the chemo- and/or radiation therapy.

ADCs will provide a means of delivering Bcl-xL inhibitors that would be difficult to deliver in unconjugated form. Due to their low cell permeability, once inside the cell, the Bcl-xL inhibitors will be unlikely to “leak” out of the cell.

### 4. DETAILED DESCRIPTION

The present disclosure concerns Bcl-xL inhibitors having low cell permeability, ADCs comprising the inhibitors, synthons useful for synthesizing the ADCs, compositions comprising the inhibitors or ADCs, and various methods of using the inhibitors and ADCs.

As will be appreciated by skilled artisans, the ADCs disclosed herein are “modular” in nature. Throughout the instant disclosure, various specific embodiments of the various “modules” comprising the ADCs, as well as the synthons useful for synthesizing the ADCs, are described. As specific non-limiting examples, specific embodiments of antibodies, linkers, and Bcl-xL inhibitors that may comprise the ADCs and synthons are described. It is intended that all of the specific embodiments described may be combined with each other as though each specific combination were explicitly described individually.

It will also be appreciated by skilled artisans that the various Bcl-xL inhibitors, ADCs and/or ADC synthons described herein may be in the form of salts, and in certain embodiments, particularly pharmaceutically acceptable salts. The compounds of the present disclosure that possess a sufficiently acidic, a sufficiently basic, or both functional groups, can react with any of a number of inorganic bases, and inorganic and organic acids, to form a salt. Alternatively, compounds that are inherently charged, such as those with a quaternary nitrogen, can form a salt with an appropriate counterion, e.g., a halide such as a bromide, chloride, or fluoride.

Acids commonly employed to form acid addition salts are inorganic acids such as hydrochloric acid, hydrobromic acid, hydroiodic acid, sulfuric acid, phosphoric acid, and the like, and organic acids such as p-toluenesulfonic acid, methanesulfonic acid, oxalic acid, p-bromophenyl-sulfonic acid, carbonic acid, succinic acid, citric acid, etc. Base addition salts include those derived from inorganic bases, such as ammonium and alkali or alkaline earth metal hydroxides, carbonates, bicarbonates, and the like.

In the disclosure below, if both structural diagrams and nomenclature are included and if the nomenclature conflicts with the structural diagram, the structural diagram controls.

### 4.1. Definitions

Unless otherwise defined herein, scientific and technical terms used in connection with the present disclosure shall have the meanings that are commonly understood by those of ordinary skill in the art.

Various chemical substituents are defined below. In some instances, the number of carbon atoms in a substituent (e.g., alkyl, alkanyl, alkenyl, alkynyl, cycloalkyl, heterocyclyl, heteroaryl, and aryl) is indicated by the prefix “Cₓ-C_{y},” wherein x is the minimum and y is the maximum number of carbon atoms. Thus, for example, “C₁-C₆ alkyl” refers to an alkyl containing from 1 to 6 carbon atoms. Illustrating further, “C₃-C₈ cycloalkyl” means a saturated hydrocarbyl ring containing from 3 to 8 carbon ring atoms.

If a substituent is described as being “substituted,” a hydrogen atom on a carbon or nitrogen is replaced with a non-hydrogen group. For example, a substituted alkyl substituent is an alkyl substituent in which at least one hydrogen atom on the alkyl is replaced with a non-hydrogen group. To illustrate, monofluoroalkyl is alkyl substituted with a fluoro radical, and difluoroalkyl is alkyl substituted with two fluoro radicals. It should be recognized that if there is more than one substitution on a substituent, each substitution may be identical or different (unless otherwise stated). If a substituent is described as being “optionally substituted”, the substituent may be either (1) not substituted or (2) substituted. Possible substituents include, but are not limited to, C₁-C₆ alkyl, C₂-C₆ alkenyl, C₂-C₆ alkynyl, aryl, cycloalkyl, heterocyclyl, heteroaryl, halogen, C₁-C₆ haloalkyl, oxo, —CN, NO₂, —OR^{xa}, —OC(O)R^{z}, —OC(O)N(R^{xa})₂, —SR^{xa}, —S(O)₂R^{xa}, —S(O)₂N(R^{xa})₂, —C(O)R^{xa}, —C(O)OR^{xa}, —C(O)N(R^{xa})₂, —C(O)N(R^{xa})S(O)₂R^{z}, —N(R^{xa})₂, —N(R^{xa})C(O)R^{z}, —N(R^{xa})S(O)₂R^{z}, —N(R^{xa})C(O)O(R^{z}), —N(R^{xa})C(O)N(R^{xa})₂, —N(R^{xa})S(O)₂N(R^{xa})₂, —(C₁-C₆ alkylenyl)-CN, —(C₁-C₆ alkylenyl)-OR^{xa}, —(C₁-C₆ alkylenyl)-OC(O)R^{z}, —(C₁-C₆ alkylenyl)-OC(O)N(R^{xa})₂, —(C₁-C₆ alkylenyl)-SR^{xa}, —(C₁-C₆ alkylenyl)-S(O)₂R^{xa}, —(C₁-C₆ alkylenyl)-S(O)₂N(R^{xa})₂, —(C₁-C₆ alkylenyl)-C(O)R^{xa}, —(C₁-C₆ alkylenyl)-C(O)OR^{xa}, —(C₁-C₆ alkylenyl)-C(O)N(R^{xa})₂, —(C₁-C₆ alkylenyl)-C(O)N(R^{xa})S(O)₂R^{z}, —(C₁-C₆ alkylenyl)-N(R^{xa})₂, —(C₁-C₆ alkylenyl)-N(R^{xa})C(O)R^{z}, —(C₁-C₆ alkylenyl)-N(R^{xa})S(O)₂R^{z}, —(C₁-C₆ alkylenyl)-N(R^{xa})C(O)O(R^{z}), —(C₁-C₆ alkylenyl)-N(R^{xa})C(O)N(R^{xa})₂, or —(C₁-C₆ alkylenyl)-N(R^{xa})S(O)₂N(R^{xa})₂; wherein R^{xa}, at each occurrence, is independently hydrogen, aryl, cycloalkyl, heterocyclyl, heteroaryl, C₁-C₆ alkyl, or C₁-C₆ haloalkyl; and R^{z}, at each occurrence, is independently aryl, cycloalkyl, heterocyclyl, heteroaryl, C₁-C₆ alkyl or C₁-C₆ haloalkyl.

Various Bcl-xL inhibitors, ADCs, and synthons are described in some embodiments herein by reference to structural formulae including substituent groups. It is to be understood that the various groups comprising the substituents may be combined as valence and stability permit. Combinations of substituents and variables envisioned by this disclosure are only those that result in the formation of stable compounds. As used herein, the term “stable” refers to compounds that possess stability sufficient to allow manufacture and that maintain the integrity of the compound for a sufficient period of time to be useful for the purpose detailed herein.

As used herein, the following terms are intended to have the following meanings:

The term “alkoxy” refers to a group of the formula —OR^{a}, where R^{a′} is an alkyl group. Representative alkoxy groups include methoxy, ethoxy, propoxy, tert-butoxy and the like.

The term “alkoxyalkyl” refers to an alkyl group substituted with an alkoxy group and may be represented by the general formula —R^{b}OR^{a} where R^{b} is an alkylene group and R^{a} is an alkyl group.

The term “alkyl” by itself or as part of another substituent refers to a saturated or unsaturated branched, straight-chain or cyclic monovalent hydrocarbon radical that is derived by the removal of one hydrogen atom from a single carbon atom of a parent alkane, alkene or alkyne. Typical alkyl groups include, but are not limited to, methyl; ethyls such as ethanyl, ethenyl, ethynyl; propyls such as propan-1-yl, propan-2-yl, cyclopropan-1-yl, prop-1-en-1-yl, prop-1-en-2-yl, prop-2-en-1-yl, cycloprop-1-en-1-yl; cycloprop-2-en-1-yl, prop-1-yn-1-yl, prop-2-yn-1-yl, etc.; butyls such as butan-1-yl, butan-2-yl, 2-methyl-propan-1-yl, 2-methyl-propan-2-yl, cyclobutan-1-yl, but-1-en-1-yl, but-1-en-2-yl, 2-methyl-prop-1-en-1-yl, but-2-en-1-yl, but-2-en-2-yl, buta-1,3-dien-1-yl, buta-1,3-dien-2-yl, cyclobut-1-en-1-yl, cyclobut-1-en-3-yl, cyclobuta-1,3-dien-1-yl, but-1-yn-1-yl, but-1-yn-3-yl, but-3-yn-1-yl, etc.; and the like. Where specific levels of saturation are intended, the nomenclature “alkanyl,” “alkenyl” and/or “alkynyl” is used, as defined below. The term “lower alkyl” refers to alkyl groups with 1 to 6 carbons.

The term “alkanyl” by itself or as part of another substituent refers to a saturated branched, straight-chain or cyclic alkyl derived by the removal of one hydrogen atom from a single carbon atom of a parent alkane. Typical alkanyl groups include, but are not limited to, methyl; ethanyl; propanyls such as propan-1-yl, propan-2-yl (isopropyl), cyclopropan-1-yl, etc.; butanyls such as butan-1-yl, butan-2-yl (sec-butyl), 2-methyl-propan-1-yl (isobutyl), 2-methyl-propan-2-yl (t-butyl), cyclobutan-1-yl, etc.; and the like.

The term “alkenyl” by itself or as part of another substituent refers to an unsaturated branched, straight-chain or cyclic alkyl having at least one carbon-carbon double bond derived by the removal of one hydrogen atom from a single carbon atom of a parent alkene. Typical alkenyl groups include, but are not limited to, ethenyl; propenyls such as prop-1-en-1-yl, prop-1-en-2-yl, prop-2-en-1-yl, prop-2-en-2-yl, cycloprop-1-en-1-yl; cycloprop-2-en-1-yl; butenyls such as but-1-en-1-yl, but-1-en-2-yl, 2-methyl-prop-1-en-1-yl, but-2-en-1-yl, but-2-en-2-yl, buta-1,3-dien-1-yl, buta-1,3-dien-2-yl, cyclobut-1-en-1-yl, cyclobut-1-en-3-yl, cyclobuta-1,3-dien-1-yl, etc.; and the like.

The term “alkynyl” by itself or as part of another substituent refers to an unsaturated branched, straight-chain or cyclic alkyl having at least one carbon-carbon triple bond derived by the removal of one hydrogen atom from a single carbon atom of a parent alkyne. Typical alkynyl groups include, but are not limited to, ethynyl; propynyls such as prop-1-yn-1-yl, prop-2-yn-1-yl, etc.; butynyls such as but-1-yn-1-yl, but-1-yn-3-yl, but-3-yn-1-yl, etc.; and the like.

The term “alkylamine” refers to a group of the formula —NHR^{a} and “dialkylamine” refers to a group of the formula —NR^{a}R^{a}, where each R^{a} is, independently of the others, an alkyl group.

The term “alkylene” refers to an alkane, alkene or alkyne group having two terminal monovalent radical centers derived by the removal of one hydrogen atom from each of the two terminal carbon atoms. Typical alkylene groups include, but are not limited to, methylene; and saturated or unsaturated ethylene; propylene; butylene; and the like. The term “lower alkylene” refers to alkylene groups with 1 to 6 carbons.

The term “aryl” means an aromatic carbocyclyl containing from 6 to 14 carbon ring atoms. An aryl may be monocyclic or polycyclic (i.e., may contain more than one ring). In the case of polycyclic aromatic rings, only one ring the polycyclic system is required to be aromatic while the remaining ring(s) may be saturated, partially saturated or unsaturated. Examples of aryls include phenyl, naphthalenyl, indenyl, indanyl, and tetrahydronaphthyl.

The term “arylene” refers to an aryl group having two monovalent radical centers derived by the removal of one hydrogen atom from each of the two ring carbons. An exemplary arylene group is a phenylene.

An alkyl group may be substituted by a “carbonyl” which means that two hydrogen atoms from a single alkanylene carbon atom are removed and replaced with a double bond to an oxygen atom.

The prefix “halo” indicates that the substituent which includes the prefix is substituted with one or more independently selected halogen radicals. For example, haloalkyl means an alkyl substituent in which at least one hydrogen radical is replaced with a halogen radical. Typical halogen radicals include chloro, fluoro, bromo and iodo. Examples of haloalkyls include chloromethyl, 1-bromoethyl, fluoromethyl, difluoromethyl, trifluoromethyl, and 1,1,1-trifluoroethyl. It should be recognized that if a substituent is substituted by more than one halogen radical, those halogen radicals may be identical or different (unless otherwise stated).

The term “haloalkoxy” refers to a group of the formula —OR^{c}, where R^{c} is a haloalkyl.

The terms “heteroalkyl,” “heteroalkanyl,” “heteroalkenyl,” “heteroalkynyl,” and “heteroalkylene” refer to alkyl, alkanyl, alkenyl, alkynyl, and alkylene groups, respectively, in which one or more of the carbon atoms, e.g., 1, 2 or 3 carbon atoms, are each independently replaced with the same or different heteroatoms or heteroatomic groups. Typical heteroatoms and/or heteroatomic groups which can replace the carbon atoms include, but are not limited to, —O—, —S—, —S—O—, —NR—, —PH, —S(O)—, —S(O)₂—, —S(O)NR^{c}—, —S(O)₂NR^{c}—, and the like, including combinations thereof, where each R^{c} is independently hydrogen or C₁-C₆ alkyl. The term “lower heteroalkyl” refers to between 1 and 4 carbon atoms and between 1 and 3 heteroatoms. The term “lower heteroalkylene” refers to alkylene groups with 1 to 4 carbon atoms and 1 to 3 heteroatoms.

The terms “cycloalkyl” and “heterocyclyl” refer to cyclic versions of “alkyl” and “heteroalkyl” groups, respectively. For heterocyclyl groups, a heteroatom can occupy the position that is attached to the remainder of the molecule. A cycloalkyl or heterocyclyl ring may be a single-ring (monocyclic) or have two or more rings (bicyclic or polycyclic).

Monocyclic cycloalkyl and heterocyclyl groups will typically contains from 3 to 7 ring atoms, more typically from 3 to 6 ring atoms, and even more typically 5 to 6 ring atoms. Examples of cycloalkyl groups include, but are not limited to, cyclopropyl; cyclobutyls such as cyclobutanyl and cyclobutenyl; cyclopentyls such as cyclopentanyl and cyclopentenyl; cyclohexyls such as cyclohexanyl and cyclohexenyl; and the like. Examples of monocyclic heterocyclyls include, but are not limited to, oxetane, furanyl, dihydrofuranyl, tetrahydrofuranyl, tetrahydropyranyl, thiophenyl (thiofuranyl), dihydrothiophenyl, tetrahydrothiophenyl, pyrrolyl, pyrrolinyl, pyrrolidinyl, imidazolyl, imidazolinyl, imidazolidinyl, pyrazolyl, pyrazolinyl, pyrazolidinyl, triazolyl, tetrazolyl, oxazolyl, oxazolidinyl, isoxazolidinyl, isoxazolyl, thiazolyl, isothiazolyl, thiazolinyl, isothiazolinyl, thiazolidinyl, isothiazolidinyl, thiodiazolyl, oxadiazolyl (including 1,2,3-oxadiazolyl, 1,2,4-oxadiazolyl, 1,2,5-oxadiazolyl (furazanyl), or 1,3,4-oxadiazolyl), oxatriazolyl (including 1,2,3,4-oxatriazolyl or 1,2,3,5-oxatriazolyl), dioxazolyl (including 1,2,3-dioxazolyl, 1,2,4-dioxazolyl, 1,3,2-dioxazolyl, or 1,3,4-dioxazolyl), 1,4-dioxanyl, dioxothiomorpholinyl, oxathiazolyl, oxathiolyl, oxathiolanyl, pyranyl, dihydropyranyl, thiopyranyl, tetrahydrothiopyranyl, pyridinyl (azinyl), piperidinyl, diazinyl (including pyridazinyl (1,2-diazinyl), pyrimidinyl (1,3-diazinyl), or pyrazinyl (1,4-diazinyl)), piperazinyl, triazinyl (including 1,3,5-triazinyl, 1,2,4-triazinyl, and 1,2,3-triazinyl)), oxazinyl (including 1,2-oxazinyl, 1,3-oxazinyl, or 1,4-oxazinyl)), oxathiazinyl (including 1,2,3-oxathiazinyl, 1,2,4-oxathiazinyl, 1,2,5-oxathiazinyl, or 1,2,6-oxathiazinyl)), oxadiazinyl (including 1,2,3-oxadiazinyl, 1,2,4-oxadiazinyl, 1,4,2-oxadiazinyl, or 1,3,5-oxadiazinyl)), morpholinyl, azepinyl, oxepinyl, thiepinyl, diazepinyl, pyridonyl (including pyrid-2(1H)-onyl and pyrid-4(1H)-onyl), furan-2(5H)-onyl, pyrimidonyl (including pyramid-2(1H)-onyl and pyramid-4(3H)-onyl), oxazol-2(3H)-onyl, 1H-imidazol-2(3H)-onyl, pyridazin-3(2H)-onyl, and pyrazin-2(1H)-onyl.

Polycyclic cycloalkyl and heterocyclyl groups contain more than one ring, and bicyclic cycloalkyl and heterocyclyl groups contain two rings. The rings may be in a bridged, fused or spiro orientation. Polycyclic cycloalkyl and heterocyclyl groups may include combinations of bridged, fused and/or spiro rings. In a spirocyclic cycloalkyl or heterocyclyl, one atom is common to two different rings. An example of a spirocycloalkyl is spiro[4.5]decane and an example of a spiroheterocyclyls is a spiropyrazoline.

In a bridged cycloalkyl or heterocyclyl, the rings share at least two common non-adjacent atoms. Examples of bridged cycloalkyls include, but are not limited to, adamantyl and norbornanyl rings. Examples of bridged heterocyclyls include, but are not limited to, 2-oxatricyclo[3.3.1.1^{3,7}]decanyl.

In a fused-ring cycloalkyl or heterocyclyl, two or more rings are fused together, such that two rings share one common bond. Examples of fused-ring cycloalkyls include decalin, naphthylene, tetralin, and anthracene. Examples of fused-ring heterocyclyls containing two or three rings include imidazopyrazinyl (including imidazo[1,2-a]pyrazinyl), imidazopyridinyl (including imidazo[1,2-a]pyridinyl), imidazopyridazinyl (including imidazo[1,2-b]pyridazinyl), thiazolopyridinyl (including thiazolo[5,4-c]pyridinyl, thiazolo[5,4-b]pyridinyl, thiazolo[4,5-b]pyridinyl, and thiazolo[4,5-c]pyridinyl), indolizinyl, pyranopyrrolyl, 4H-quinolizinyl, purinyl, naphthyridinyl, pyridopyridinyl (including pyrido[3,4-b]-pyridinyl, pyrido[3,2-b]-pyridinyl, or pyrido[4,3-b]-pyridinyl), and pteridinyl. Other examples of fused-ring heterocyclyls include benzo-fused heterocyclyls, such as dihydrochromenyl, tetrahydroisoquinolinyl, indolyl, isoindolyl (isobenzazolyl, pseudoisoindolyl), indoleninyl (pseudoindolyl), isoindazolyl (benzpyrazolyl), benzazinyl (including quinolinyl (1-benzazinyl) or isoquinolinyl (2-benzazinyl)), phthalazinyl, quinoxalinyl, quinazolinyl, benzodiazinyl (including cinnolinyl (1,2-benzodiazinyl) or quinazolinyl (1,3-benzodiazinyl)), benzopyranyl (including chromanyl or isochromanyl), benzoxazinyl (including 1,3,2-benzoxazinyl, 1,4,2-benzoxazinyl, 2,3,1-benzoxazinyl, or 3,1,4-benzoxazinyl), benzo[d]thiazolyl, and benzisoxazinyl (including 1,2-benzisoxazinyl or 1,4-benzisoxazinyl).

The term “heteroaryl” refers to an aromatic heterocyclyl containing from 5 to 14 ring atoms. A heteroaryl may be a single ring or 2 or 3 fused rings. Examples of heteroaryls include 6-membered rings such as pyridyl, pyrazyl, pyrimidinyl, pyridazinyl, and 1,3,5-, 1,2,4- or 1,2,3-triazinyl; 5-membered ring substituents such as triazolyl, pyrrolyl, imidazoyl, furanyl, thiophenyl, pyrazolyl, oxazolyl, isoxazolyl, thiazolyl, 1,2,3-, 1,2,4-, 1,2,5-, or 1,3,4-oxadiazolyl and isothiazolyl; 6/5-membered fused ring substituents such as imidazopyrazinyl (including imidazo[1,2-a]pyrazinyl)imidazopyridinyl (including imidazo[1,2-a]pyridinyl), imidazopyridazinyl (including imidazo[1,2-b]pyridazinyl), thiazolopyridinyl (including thiazolo[5,4-c]pyridinyl, thiazolo[5,4-b]pyridinyl, thiazolo[4,5-b]pyridinyl, and thiazolo[4,5-c]pyridinyl), benzo[d]thiazolyl, benzothiofuranyl, benzisoxazolyl, benzoxazolyl, purinyl, and anthranilyl; and 6/6-membered fused rings such as benzopyranyl, quinolinyl, isoquinolinyl, cinnolinyl, quinazolinyl, and benzoxazinyl. Heteroaryls may also be heterocycles having aromatic (4N+2 pi electron) resonance contributors such as pyridonyl (including pyrid-2(1H)-onyl and pyrid-4(1H)-onyl), pyrimidonyl (including pyramid-2(1H)-onyl and pyramid-4(3H)-onyl), pyridazin-3(2H)-onyl and pyrazin-2(1H)-onyl.

The term “heterocyclene” refers to a heterocycle group having two monovalent radical centers derived by the removal of one hydrogen atom from each of the two ring atoms. Exemplary heterocyclene groups include:

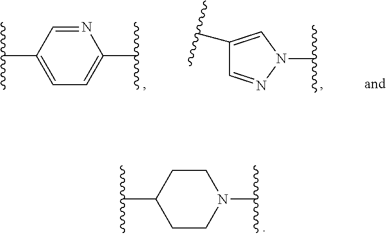 [SMILES: *c1ccc(*)nc1 ; *c1cnn(*)c1 ; *C1CCN(*)CC1]

The term “sulfonate” as used herein means a salt or ester of a sulfonic acid.

The term “methyl sulfonate” as used herein means a methyl ester of a sulfonic acid group.

The term “carboxylate” as used herein means a salt or ester of a caboxylic acid.

The term “polyol”, as used herein, means a group containing more than two hydroxyl groups independently or as a portion of a monomer unit. Polyols include, but are not limited to, reduced C₂-C₆ carbohydrates, ethylene glycol, and glycerin.

The term “sugar” when used in context of “G,” “G¹,” “G,” “G^{b},” and “R′” includes O-glycoside, N-glycoside, S-glycoside and C-glycoside (C-glycoslyl) carbohydrate derivatives of the monosaccharide and disaccharide classes and may originate from naturally-occurring sources or may be synthetic in origin. For example “sugar” when used in context of “G,” “G¹,” “G^{a},” “G^{b},” and “R′” includes derivatives such as but not limited to those derived from glucuronic acid, galacturonic acid, galactose, and glucose among others. Suitable sugar substitutions include but are not limited to hydroxyl, amine, carboxylic acid, sulfonic acid, phosphonic acid, esters, and ethers.

The term “NHS ester” means the N-hydroxysuccinimide ester derivative of a carboxylic acid.

The term “amine” when used in context of “G,” “G^{a},” “G^{b},” and “R′” includes primary, secondary and tertiary aliphatic amines, including cyclic versions, that contain a nitrogen atom of sufficient basicity to render the pKa of its conjugate acid greater than or equal to approximately 7. The term “amine” when used in context of “G,” “G^{a},” “G^{b},” and “R′” is also contemplated to include a quanidine moiety, —NHC(NH₂)₂.

The term “salt” when used in context of “G,” “G^{a},” “G^{b},” and “R′” includes but is not limited to quaternary ammonium cations and their associated counter-ions, zwitter ions, which carry internally both cationic and anionic charges but are neutral overall, and dipolar moieties such as amine oxide, which carry formal charges.

The term salt when used in context of “or salt thereof” includes salts commonly used to form alkali metal salts and to form addition salts of free acids or free bases. In general, these salts typically may be prepared by conventional means by reacting, for example, the appropriate acid or base with a compound of the invention.

Where a salt is intended to be administered to a patient (as opposed to, for example, being in use in an in vitro context), the salt preferably is pharmaceutically acceptable and/or physiologically compatible. The term “pharmaceutically acceptable” is used adjectivally in this patent application to mean that the modified noun is appropriate for use as a pharmaceutical product or as a part of a pharmaceutical product. The term “pharmaceutically acceptable salt” includes salts commonly used to form alkali metal salts and to form addition salts of free acids or free bases. In general, these salts typically may be prepared by conventional means by reacting, for example, the appropriate acid or base with a compound of the invention.

### 4.2. Exemplary Embodiments

As noted in the Summary, aspects of the disclosure concern Bcl-xL inhibitors having low cell permeability and ADCs comprising Bcl-xL inhibitors linked to antibodies by way of linkers. In specific embodiments, the ADCs are compounds according to structural formula (I), below, or salts thereof, wherein Ab represents the antibody, D represents a Bcl-xL inhibitor (drug), L represents a linker, LK represents a linkage formed between a reactive functional group on linker L and a complementary functional group on antibody Ab and m represents the number of D-L-LK units linked to the antibody:

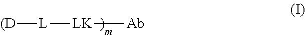

Specific embodiments of various Bcl-xL inhibitors per se, and various Bcl-xL inhibitors (D), linkers (L) and antibodies (Ab) that can comprise the ADCs described herein, as well as the number of Bcl-xL inhibitors linked to the ADCs, are described in more detail below.

### 4.3. Bcl-xL INHIBITORS

One aspect of the instant disclosure concerns Bcl-xL inhibitors that have low cell permeability. The compounds are generally heterocyclic in nature and include one or more solubilizing groups that impart the compounds with high water solubility and low cell permeability. The solubilizing groups are generally groups that are capable of hydrogen bonding, forming dipole-dipole interactions, and/or that include a polyethylene glycol polymer containing from 1 to 30 units, one or more polyols, one or more salts, or one or more groups that are charged at physiological pH.

The Bcl-xL inhibitors may be used as compounds or salts per se in the various methods described herein, or may be included as a component part of an ADC.

Specific embodiments of Bcl-xL inhibitors that may be used in unconjugated form, or that may be included as part of an ADC include compounds according to structural formulae (IIa), (IIb), (IIc), or (IId):

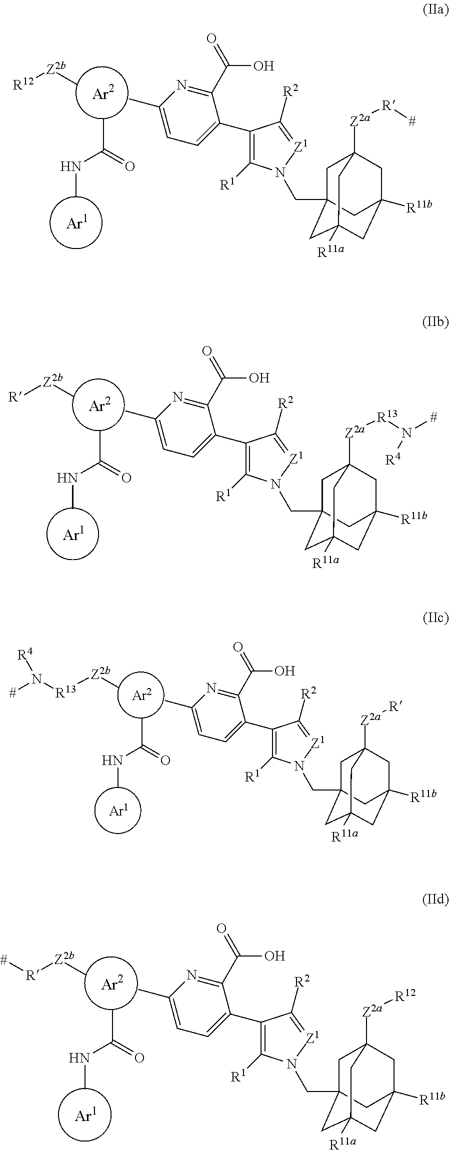

or salts thereof, wherein:
Ar¹ is selected from

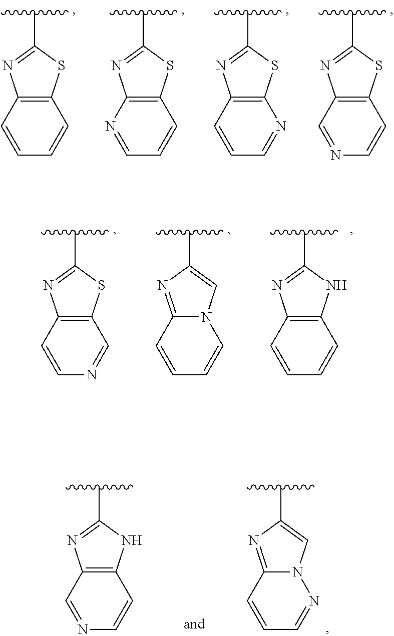

and, and is optionally substituted with one or more substituents independently selected from halo, hydroxy, nitro, lower alkyl, lower heteroalkyl, alkoxy, amino, cyano and halomethyl;
Ar² is selected from

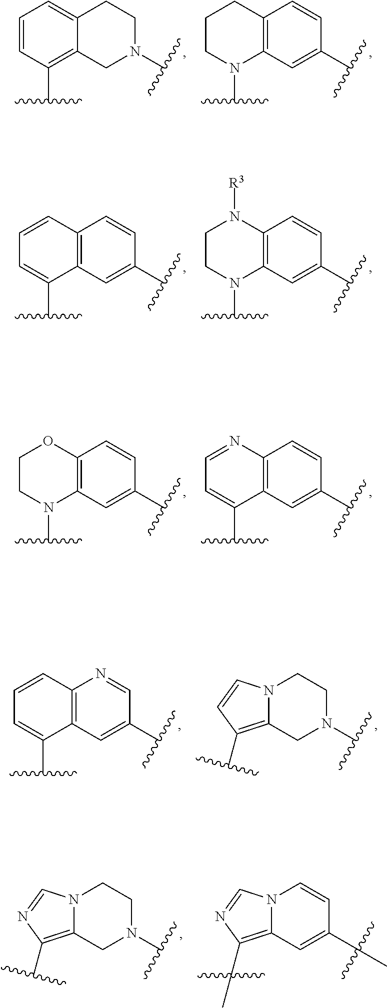

and is optionally substituted with one or more substitutents independently selected from halo, hydroxy, nitro, lower alkyl, lower heteroalkyl, alkoxy, amino, cyano and halomethyl, wherein the R¹²—Z^{2b}—, R′—Z^{2b}—, #—N(R⁴)—R¹³—Z^{2b}—, or #—R′—Z^{2b}— substituents are attached to Ar² at any Ar² atom capable of being substituted;
Z¹ is selected from N, CH, C-halo, C—CH₃ and C—CN;
Z^{2a} and Z^{2b} are each, independently from one another, selected from a bond, NR⁶, CR^{6a}R^{6b}, O, S, S(O), SO₂, —NR⁶C(O)—, —NR^{6a}C(O)NR^{6b}—, and —NR⁶C(O)O—;
R′ is a alkylene, heteroalkylene, cycloalkylene, heterocyclene, aryl or heteroaryl independently substituted at one or more carbon or heteroatoms with a solubilizing moiety containing a group selected from a polyol, a polyethylene glycol containing from 4 to 30 ethylene glycol units, a salt, and a group that is charged at physiological pH and combinations thereof, wherein #, where attached to R′, is attached to R′ at any R′ atom capable of being substituted;
R¹ is selected from hydrogen, methyl, halo, halomethyl, ethyl, and cyano;
R² is selected from hydrogen, methyl, halo, halomethyl and cyano;
R³ is selected from hydrogen, methyl, ethyl, halomethyl and haloethyl;
R⁴ is selected from hydrogen, lower alkyl and lower heteroalkyl or is taken together with an atom of R¹³ to form a cycloalkyl or heterocyclyl ring having between 3 and 7 ring atoms;
R⁶, R^{6a} and R^{6b} are each, independent from one another, selected from hydrogen, optionally substituted lower alkyl, optionally substituted lower heteroalkyl, optionally substituted cycloalkyl and optionally substituted heterocyclyl, or are taken together with an atom from R⁴ and at atom from R¹³ to form a cycloalkyl or heterocyclyl ring having between 3 and 7 ring atoms;
R^{11a} and R^{11b} are each, independently of one another, selected from hydrogen, halo, methyl, ethyl, halomethyl, hydroxyl, methoxy, CN, and SCH₃;
R¹² is optionally R′ or is selected from hydrogen, halo, cyano, optionally substituted alkyl, optionally substituted heteroalkyl, optionally substituted heterocyclyl, and optionally substituted cycloalkyl;
R¹³ is selected from optionally substituted alkylene, optionally substituted heteroalkylene, optionally substituted heterocyclene, and optionally substituted cycloalkylene; and
#represents the point of attachment to a linker L or a hydrogen atom.

One embodiment of Bcl-xL inhibitors that may be used in unconjugated form, or that may be included as part of an ADC include compounds according to structural formulae (IIa), (IIb), (IIc), or (IId):

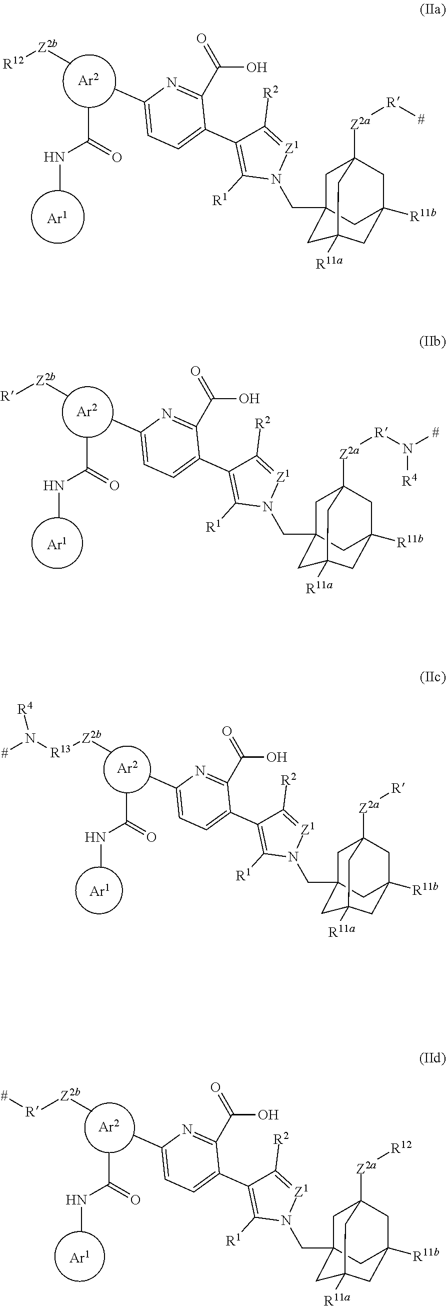

or salts thereof, wherein:
Ar¹ is selected from

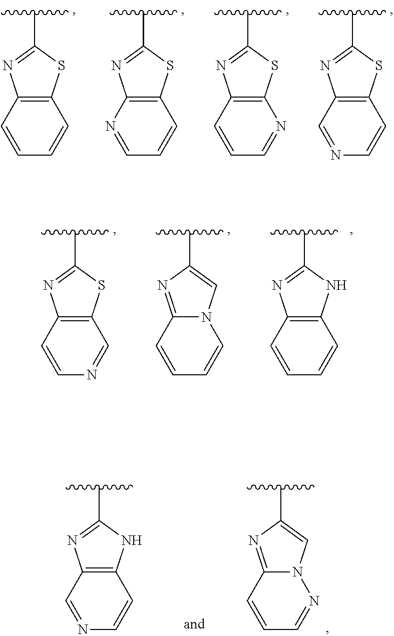

and is optionally substituted with one or more substituents independently selected from halo, hydroxy, nitro, lower alkyl, lower heteroalkyl, alkoxy, amino, cyano and halomethyl;
Ar² is selected from

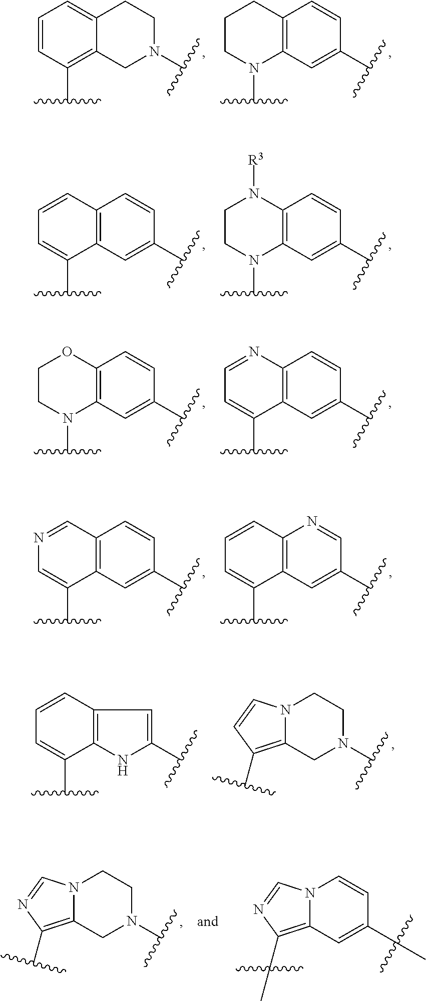

and is optionally substituted with one or more substitutents independently selected from halo, hydroxy, nitro, lower alkyl, lower heteroalkyl, alkoxy, amino, cyano and halomethyl, wherein the R¹²—Z^{2b}—, R′—Z^{2b}—, #—N(R⁴)—R¹³—Z^{2b}—, or #—R′—Z^{2b}-substituents are attached to Ar² at any Ar² atom capable of being substituted;
Z¹ is selected from N, CH, C-halo, C—CH₃ and C—CN;
Z^{2a} and Z^{2b} are each, independently from one another, selected from a bond, NR⁶, CR^{6a}R^{6b}, O, S, S(O), SO₂, —NR⁶C(O)—, —NR^{6a}C(O)NR^{6b}—, and —NR⁶C(O)O—;
R′ is

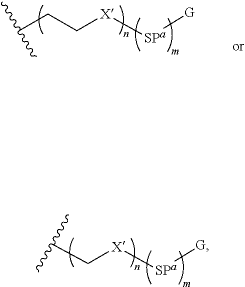

or wherein #, where attached to R′, is attached to R′ at any R′ atom capable of being substituted;
X′ is selected at each occurrence from —N(R¹⁰)—, —N(R¹⁰)C(O)—, —N(R¹⁰)S(O)₂—, —S(O)₂N(R¹⁰)—, and —O—;
n is selected from 0-3;
R¹⁰ is independently selected at each occurrence from hydrogen, alkyl, heterocycle, aminoalkyl, G-alkyl, heterocycle, and —(CH₂)₂—O—(CH₂)₂—O—(CH₂)₂—NH₂;
G at each occurrence is independently selected from a polyol, a polyethylene glycol with between 4 and 30 repeating units, a salt and a moiety that is charged at physiological pH;
SP^{a} is independently selected at each occurrence from oxygen, —S(O)₂N(H)—, —N(H)S(O)₂—, —N(H)C(O)—, —C(O)N(H)—, —N(H)—, arylene, heterocyclene, and optionally substituted methylene; wherein methylene is optionally substituted with one or more of —NH(CH₂)₂G, amine, alkyl, and carbonyl;
m is selected from 0-12;
R¹ is selected from hydrogen, methyl, halo, halomethyl, ethyl, and cyano;
R² is selected from hydrogen, methyl, halo, halomethyl and cyano;
R³ is selected from hydrogen, methyl, ethyl, halomethyl and haloethyl;
R⁴ is selected from hydrogen, lower alkyl and lower heteroalkyl or is taken together with an atom of R¹³ to form a cycloalkyl or heterocyclyl ring having between 3 and 7 ring atoms;
R⁶, R^{6a} and R^{6b} are each, independent from one another, selected from hydrogen, optionally substituted lower alkyl, optionally substituted lower heteroalkyl, optionally substituted cycloalkyl and optionally substituted heterocyclyl, or are taken together with an atom from R⁴ and at atom from R¹³ to form a cycloalkyl or heterocyclyl ring having between 3 and 7 ring atoms;
R^{11a} and R^{11b} are each, independently of one another, selected from hydrogen, halo, methyl, ethyl, halomethyl, hydroxyl, methoxy, CN, and SCH₃;
R¹² is optionally R′ or is selected from hydrogen, halo, cyano, optionally substituted alkyl, optionally substituted heteroalkyl, optionally substituted heterocyclyl, and optionally substituted cycloalkyl;
R¹³ is selected from optionally substituted alkylene, optionally substituted heteroalkylene, optionally substituted heterocyclene, and optionally substituted cycloalkylene; and
#represents either a hydrogen atom or the point of attachment to a linker L.

When a Bcl-xL inhibitor of structural formulae (IIa)-(IId) is not a component of an ADC, #in formulae (IIa)-(IId) represents the point of attachment to a hydrogen atom. When the Bcl-xL inhibitor is a component of an ADC, #in formulae (IIa)-(IId) represents the point of attachment to the linker. When a Bcl-xL inhibitor is a component of an ADC, the ADC may comprise one or more Bcl-xL inhibitors, which may be the same or different, but are typically the same.

In certain embodiments, R′ is a C₂-C₈ heteroalkylene substituted with one or more moieties containing a salt and/or a group that is charged at physiological pH. The salt may be selected, for example, from the salt of a carboxylate, a sulfonate, a phosphonate, and an ammonium ion. For example, the salt may be the sodium or potassium salt of a carboxylate, sulfonate or phosphonate or the chloride salt of an ammonium ion. The group that is charged at physiological pH may be any group that is charged at a physiological pH, including, by way of example and not limitation, a zwitterionic group. In certain embodiments a group that is a salt is a dipolar moiety such as, but not limited to, N-oxides of amines including certain heterocyclyls such as, but not limited to, pyridine and quinoline. In specific embodiments the group that is charged at physiological pH is selected independently at each occurrence, from carboxylate, sulfonate, phosphonate, and amine.

In certain embodiments, R′ is a C₂-C₈ heteroalkylene substituted with one or more moieties containing polyethylene glycol or a polyol such as a diol or a sugar moiety.

In certain embodiments, R′ may be substituted with groups in addition to a solubilizing moiety. For example, R′ may be substituted with one or more of the same or different alkyl, heteroalkyl, cycloalkyl, heterocyclyl, aryl, heteroaryl, or halo groups.

In certain embodiments, R′ is represented by the formula:

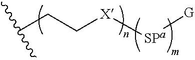

or a salt thereof, wherein:
X′ is selected at each occurrence from —N(R¹⁰)— and —O—;
n is selected from 1-3;
R¹⁰ is individually selected at each occurrence from hydrogen, alkyl, heterocycle, aminoalkyl, G-alkyl, heterocycle, and —(CH₂)₂—O—(CH₂)₂—O—(CH₂)₂—NH₂;
G at each occurrence is independently selected from a polyol, a polyethylene glycol with between 4 and 30 repeating unit (referred to herein as PEG4-30), a salt and a moiety that is charged at physiological pH;
SP^{a} is independently selected at each occurrence from oxygen, sulfonamide, arylene, heterocyclene, and optionally substituted methylene; wherein methylene is optionally substituted with one or more of —NH(CH₂)₂G, amine and carbonyl; and
m is selected from 0-6,
wherein there is at least one substitutable nitrogen in R′ that is attached to a linker or a hydrogen atom at a substitutable nitrogen atom of R′.

In certain embodiments, R′ is

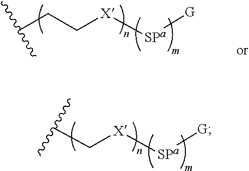

X′ is selected at each occurrence from —N(R¹⁰)—, —N(R¹⁰)C(O)—, —N(R¹⁰)S(O)₂—, —S(O)₂N(R¹⁰)—, and —O—;
n is selected from 0-3;
R¹⁰ is independently selected at each occurrence from hydrogen, alkyl, heterocycle, aminoalkyl, G-alkyl, heterocycle, and —(CH₂)₂—O—(CH₂)₂—O—(CH₂)₂—NH₂;
G at each occurrence is independently selected from a polyol, a polyethylene glycol with between 4 and 30 repeating units, a salt and a moiety that is charged at physiological pH;
SP^{a} is independently selected at each occurrence from oxygen-S(O)₂N(H)—, —N(H)S(O)₂—, —N(H)C(O)—, —C(O)N(H)—, —N(H)—, arylene, heterocyclene, and optionally substituted methylene; wherein methylene is optionally substituted with one or more of —NH(CH₂)₂G, amine, alkyl, and carbonyl;
m is selected from 0-12, and
#, where attached to R′, is attached to R′ at any R′ atom capable of being substituted.

In certain embodiments, G at each occurrence is a salt or a moiety that is charged at physiological pH.

In certain embodiments, G at each occurrence is a salt of a carboxylate, a sulfonate, a phosphonate, or ammonium.

In certain embodiments, G at each occurrence is a moiety that is charged at physiological pH selected from the group consisting of carboxylate, a sulfonate, a phosphonate, and an amine.

In certain embodiments, G at each occurrence is a moiety containing a polyethylene glycol or a polyol.

In certain embodiments, the polyol is a sugar.

In certain embodiments, R′ includes at least one substitutable nitrogen suitable for attachment to a linker.

In certain embodiments, G is selected independently at each occurrence from:

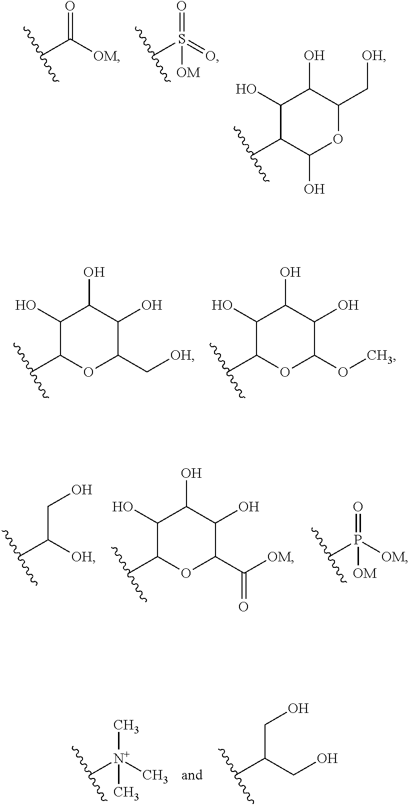

wherein M is hydrogen or a positively charged counterion. In certain embodiments, M is Na⁺, K⁺ or Li⁺. In certain embodiments, M is hydrogen. In particular embodiments, G is SO₃H.

In certain embodiments, G is selected independently at each occurrence from:

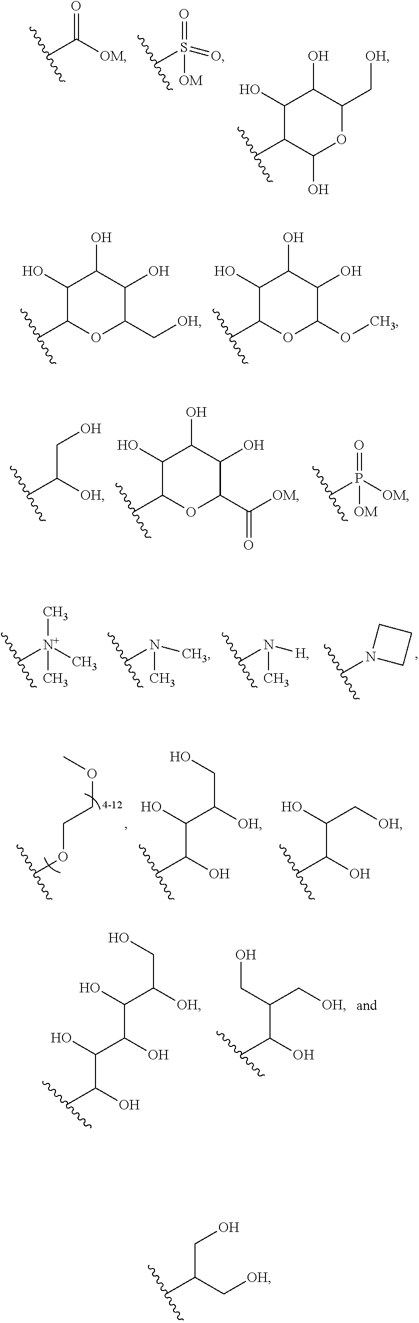

wherein M is hydrogen or a positively charged counterion. In certain embodiments, M is hydrogen. In particular embodiments, G is SO₃H.

In certain embodiments, R′ is selected from:

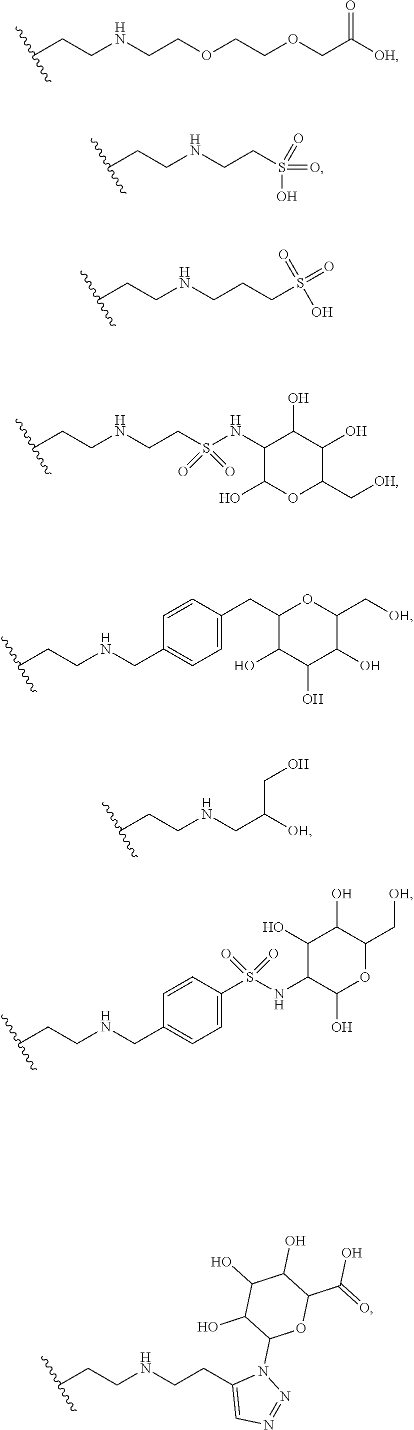

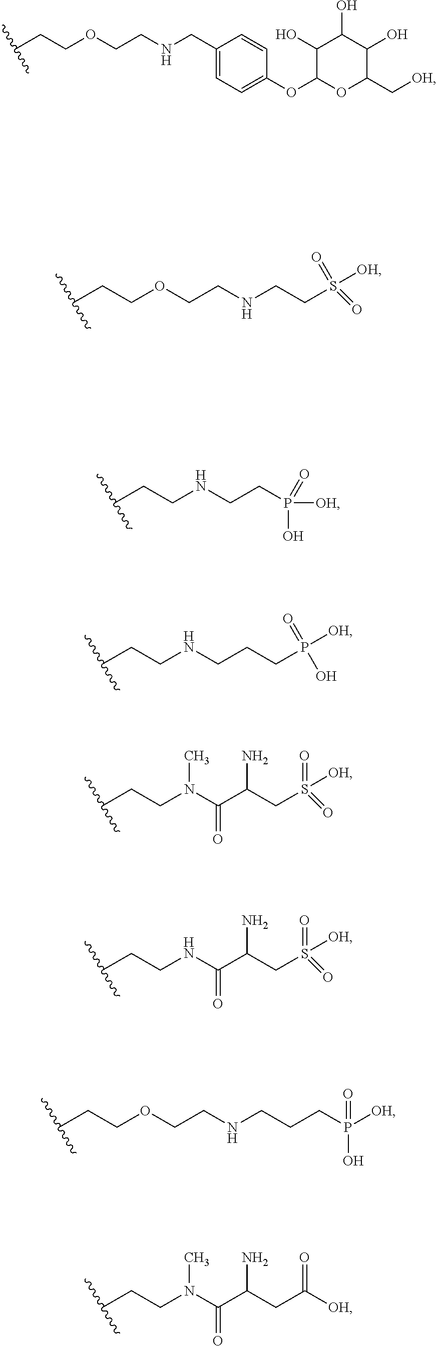

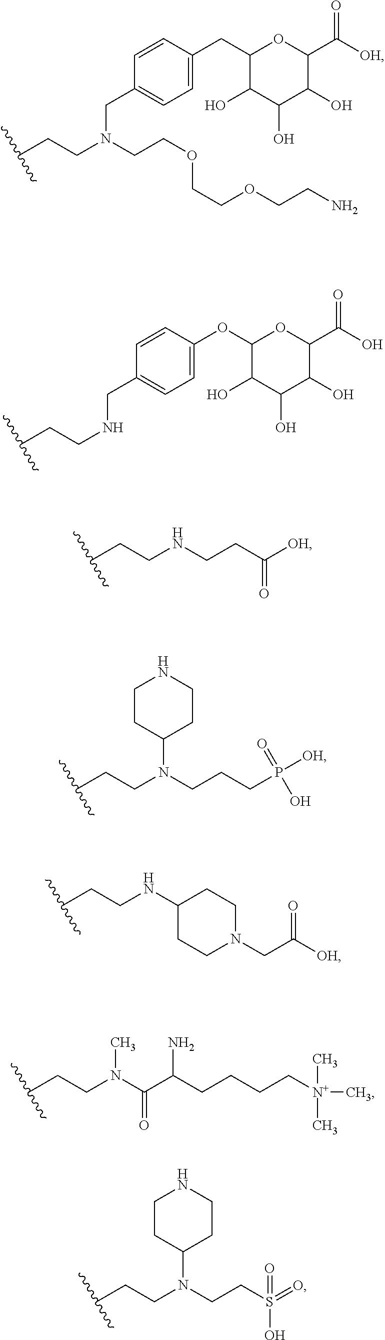

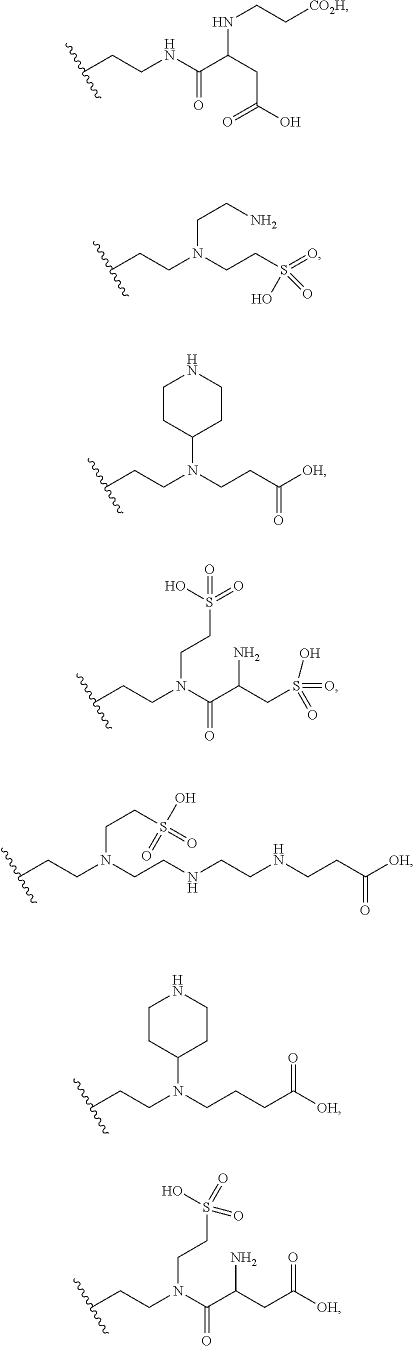

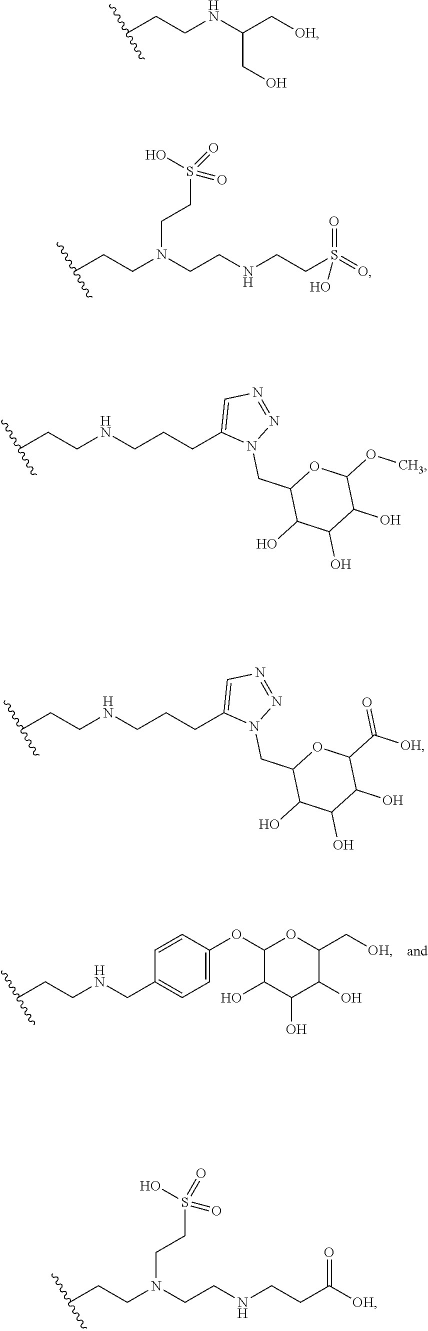

or a salt thereof. When Bcl-xL inhibitors of this embodiment are included in an ADC, the linker of the ADC is linked to the nitrogen atom of an available primary or secondary amine group.

In certain embodiments, R′ is selected from:

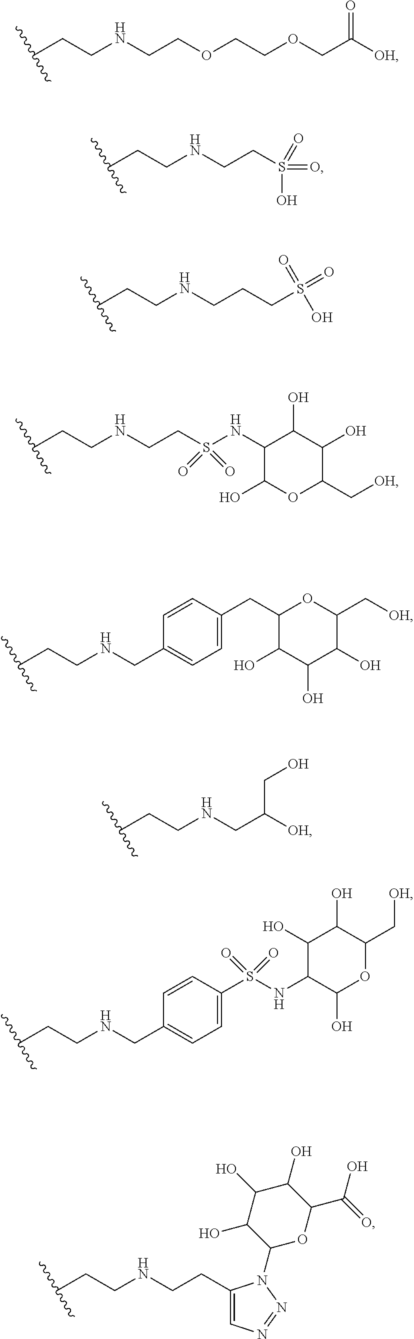

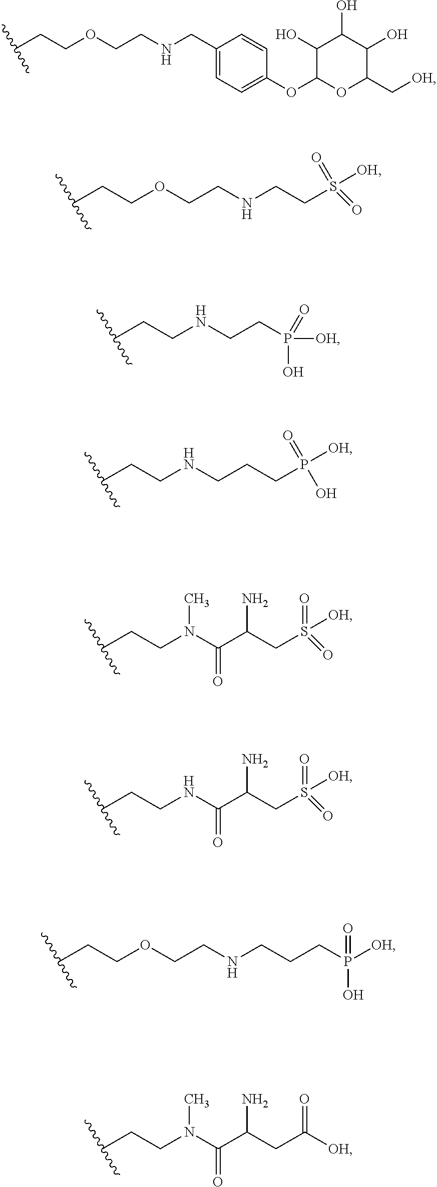

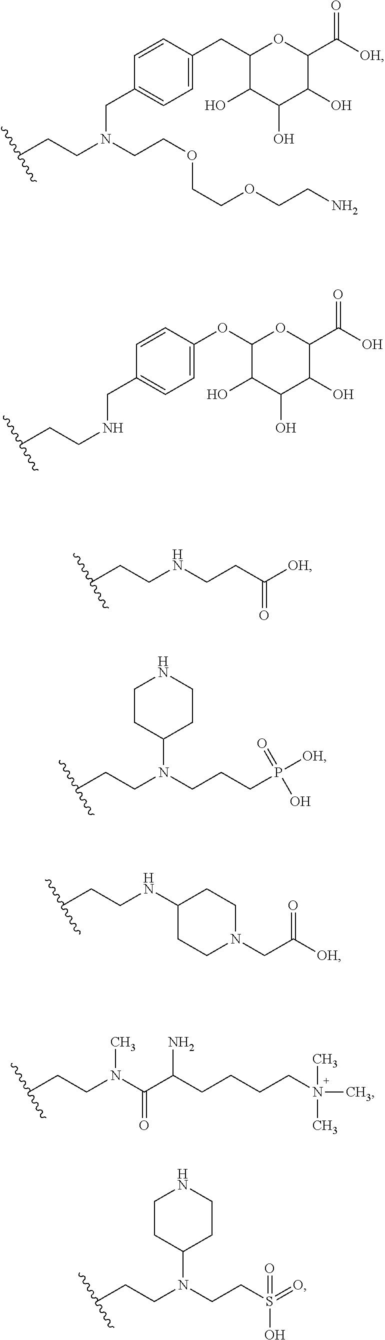

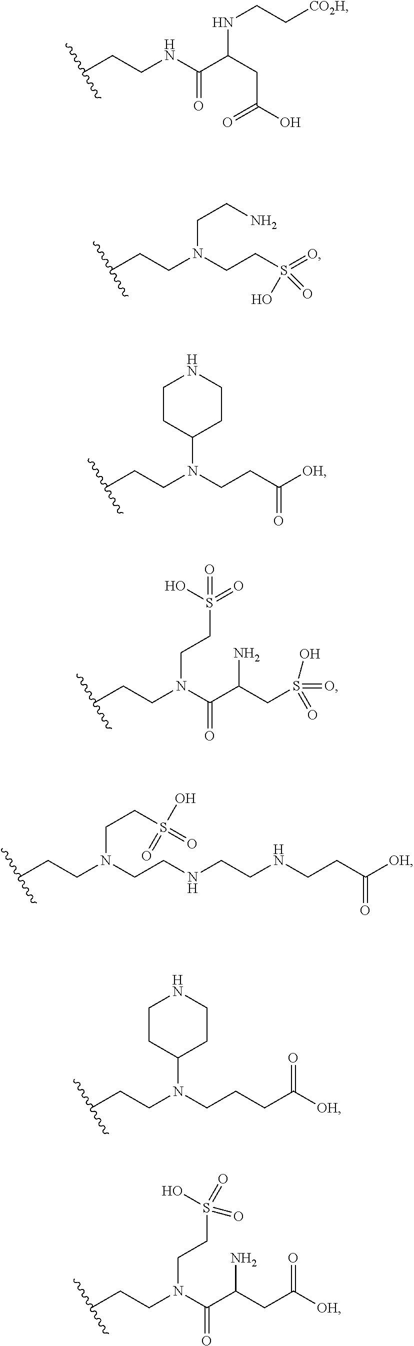

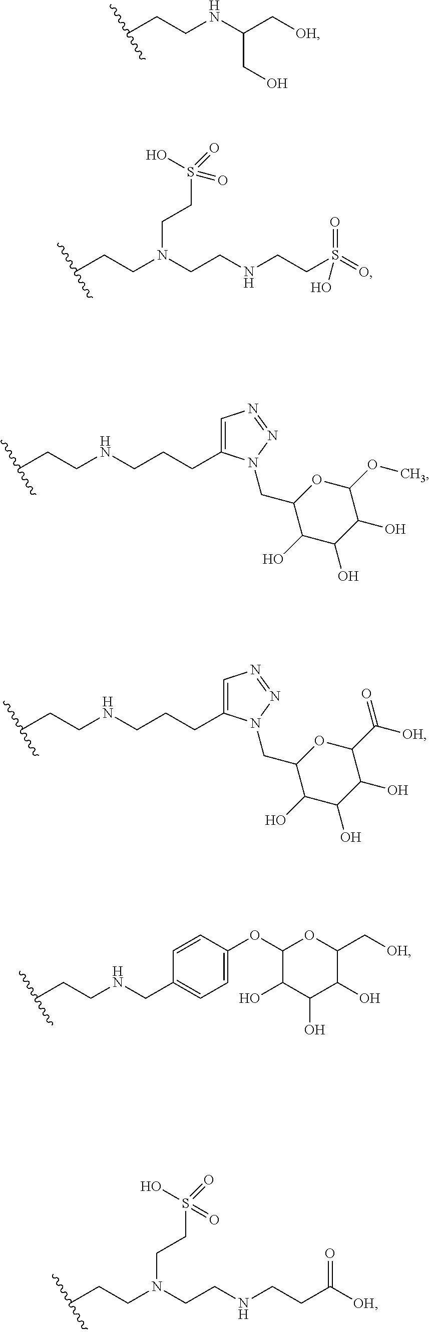

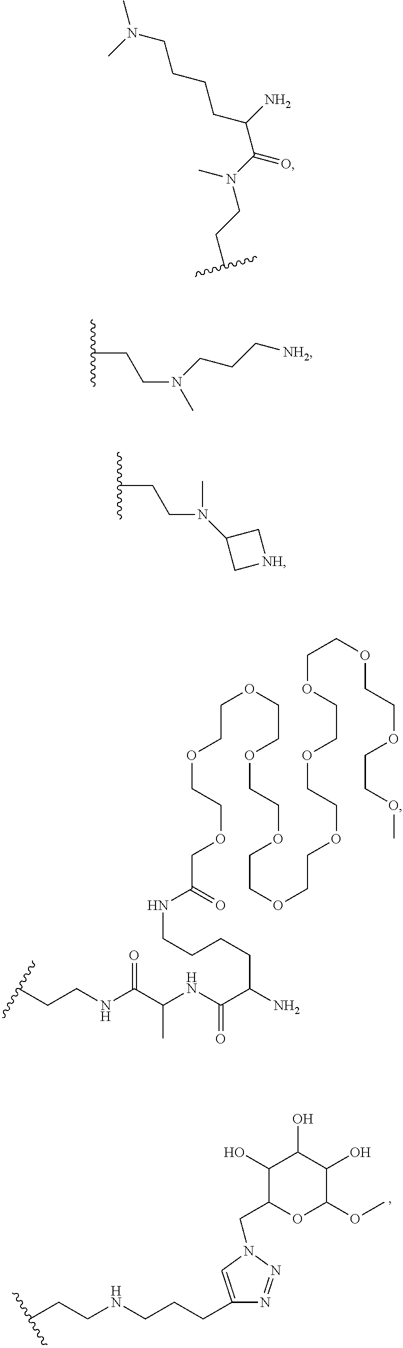

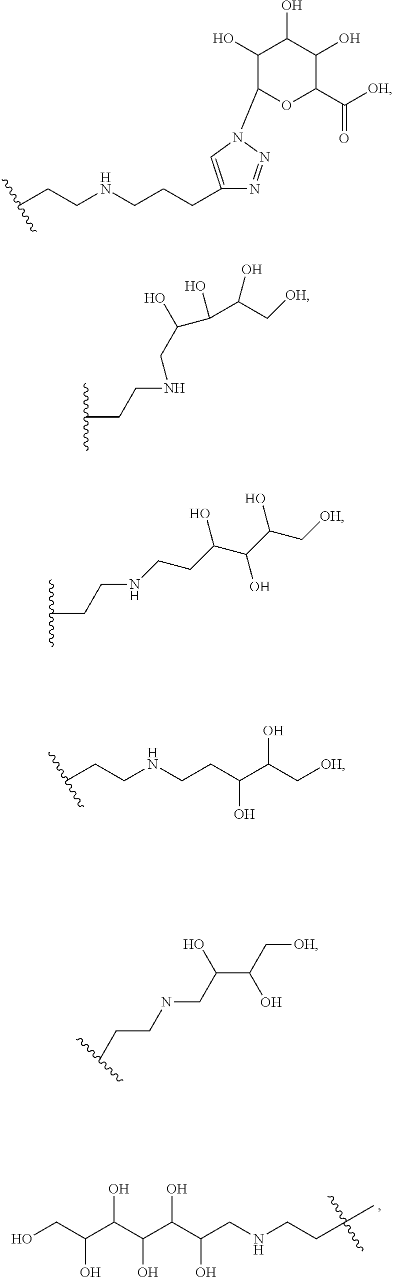 [SMILES: *CCNCCCc1cn(C2OC(C(=O)O)C(O)C(O)C2O)nn1 ; *CCNCC(O)C(O)C(O)CO ; *CCNCCC(O)C(O)C(O)CO ; *CCNCCC(O)C(O)CO ; *CCNCC(O)C(O)CO ; *CCNCC(O)C(O)C(O)C(O)C(O)CO]

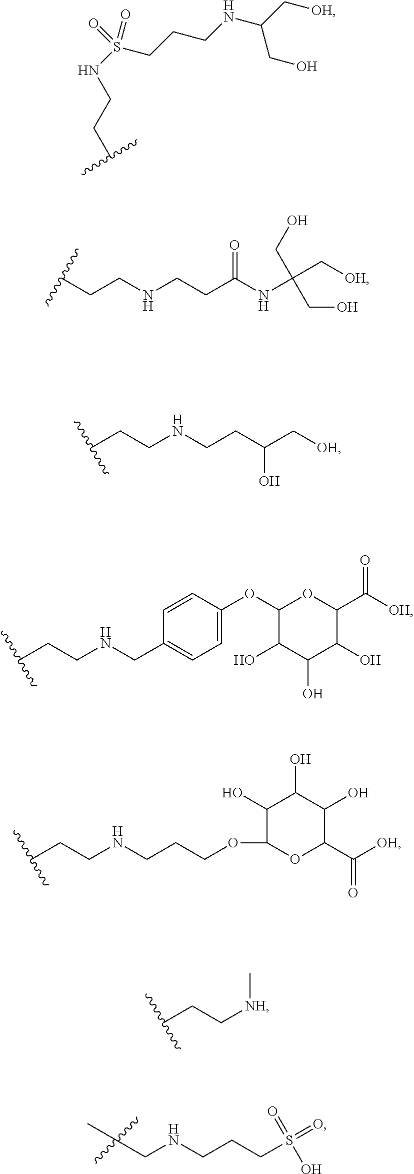

or r a salt thereof. When Bcl-xL inhibitors of this embodiment are included in an ADC, the linker of the ADC is linked to the nitrogen atom of an available primary or secondary amine group.

In certain embodiments, Ar¹ of formulae (IIa)-(IId) is selected from

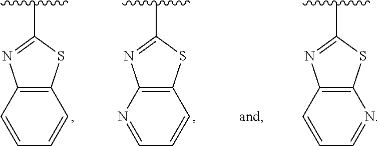 [SMILES: *c1nc2ccccc2s1 ; *c1nc2ncccc2s1 ; *c1nc2cccnc2s1]

In certain embodiments, Ar¹ of formulae (IIa)-(IId) is selected from

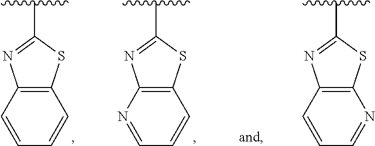 [SMILES: *c1nc2ccccc2s1 ; *c1nc2ncccc2s1 ; *c1nc2cccnc2s1]

and is optionally substituted with one or more substituents independently selected from halo, cyano, methyl, and halomethyl. In particular embodiments, Ar¹ is

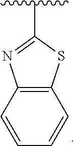 [SMILES: *c1nc2ccccc2s1]

In certain embodiments, Ar² is

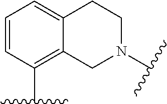 [SMILES: *c1cccc2c1CN(*)CC2]

optionally substituted with one or more substituents, wherein the R¹²—Z^{2b}—, R′—Z^{2b}—, #—N(R⁴)—R¹³—Z^{2b}—, or #—R′—Z^{2b}— substituents are attached to Ar² at any Ar² atom capable of being substituted. In certain embodiments, Ar² is selected from:

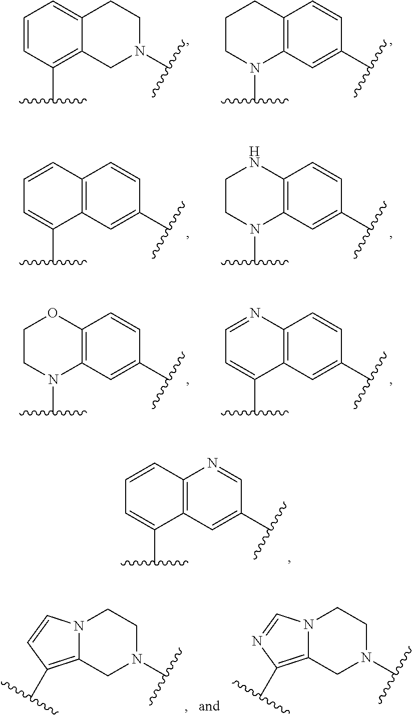

and is optionally substituted with one or more substituents, wherein the R¹²—Z^{2b}—, R′—Z^{2b}—, #—N(R⁴)—R¹³—Z^{2b}—, or #—R′—Z^{b}-substituents are attached to Ar² at any Ar² atom capable of being substituted. In certain embodiments, Ar² is selected from:

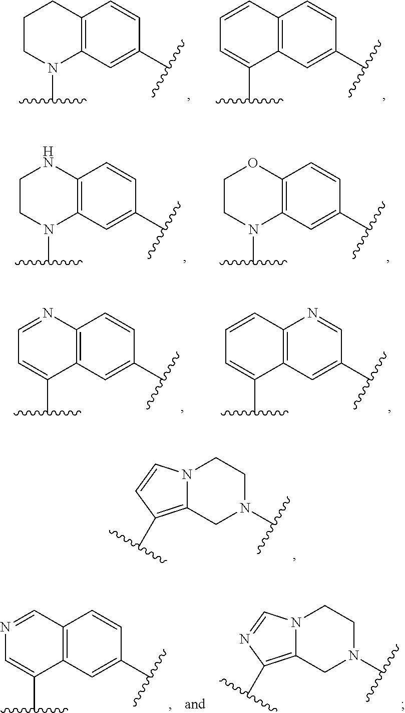

and is optionally substituted with one or more substituents, wherein the R¹²—Z^{2b}—, R′—Z^{2b}—, #—N(R⁴)—R¹³—Z^{2b}—, or #—R′—Z^{2b}— substituents are attached to Ar² at any Ar² atom capable of being substituted. In certain embodiments, Ar² is substituted with at least one solubilizing group. In certain embodiments, the solubilizing group is selected from a moiety containing a polyol, a polyethylene glycol, a salt, or a group that is charged at physiological pH.

In certain embodiments, Z¹ of formulae (IIa)-(IId) is N.

In certain embodiments, Z^{2a} of formulae (IIa)-(IId) is O. In certain embodiments, Z^{2a} of formulae (IIa)-(IId) is CR^{6a}R^{6b}. In certain embodiments, Z^{2a} of formulae (IIa)-(IId) is S. In certain embodiments, Z^{2a} of formulae (IIa)-(IId) is —NR⁶C(O)—. In particular embodiments, R⁶ is hydrogen.

In certain embodiments, Z^{2b} of formulae (IIa)-(IId) is O. In certain embodiments, Z^{2b} of formulae (IIa)-(IId) is NH.

In certain embodiments, R¹ of formulae (IIa)-(IId) is selected from methyl and chloro.

In certain embodiments, R² of formulae (IIa)-(IId) is selected from hydrogen and methyl. In particular embodiments, R² is hydrogen.

In certain embodiments the Bcl-xL inhibitor is a compound of formula (IIa). In certain embodiments in which the Bcl-xL inhibitor is a compound of formula (IIa), the compound has the structural formula (IIa.1),

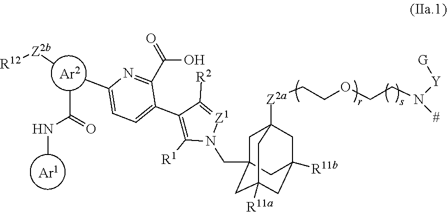

or salts thereof, wherein:
Ar¹, Ar², Z¹, Z^{2a}, Z^{2b}, R¹, R², R^{11a}, R^{11b}, R¹², G and #are defined as above;
Y is optionally substituted alkylene;
r is 0 or 1; and
s is 1, 2 or 3.

In certain embodiments in which the Bcl-xL inhibitor is a compound of formula (IIa.1), r is 0 and s is 1.

In certain embodiments in which the Bcl-xL inhibitor is a compound of formula (IIa.1), r is 0 and s is 2.

In certain embodiments in which the Bcl-xL inhibitor is a compound of formula (IIa.1), r is 1 and s is 2.

In certain embodiments in which the Bcl-xL inhibitor is a compound of formula (IIa.1), Z^{2a} is selected from O, NH, CH₂ and S. In particular embodiments, Z^{2a} is O. In certain embodiments, Z^{2a} of formula (IIa.1) is —CR^{6a}R^{6b}—. In certain embodiments, Z^{2a} of formula (IIa.1) is CH₂. In certain embodiments, Z^{2a} of formula (IIa.1) is S. In certain embodiments, Z^{2a} of formula (IIa.1) is —NR⁶C(O)—.

In certain embodiments in which the Bcl-xL inhibitor is a compound of formula (IIa.1), Y is selected from ethylene, propylene and butylene. In particular embodiments, Y is selected from ethylene and propylene.

In certain embodiments in which the Bcl-xL inhibitor is a compound of formula (IIa.1), G is selected from

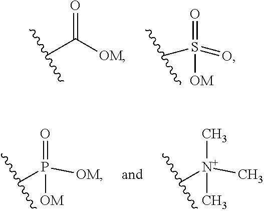

wherein M is hydrogen or a positively charged counterion. In particular embodiments, G is

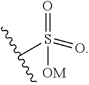

In particular embodiments, G is SO₃H.

In certain embodiments in which the Bcl-xL inhibitor is a compound of formula (IIa.1), Ar² is selected from

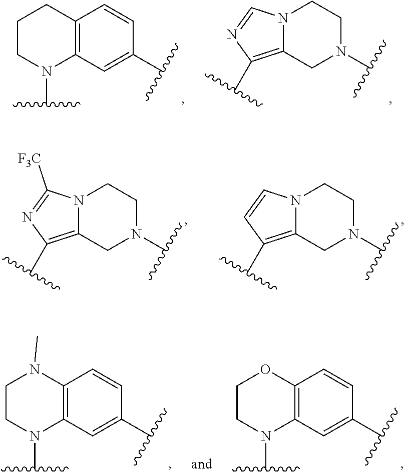 [SMILES: *c1ccc2c(c1)N(*)CCC2 ; *c1ncn2c1CN(*)CC2 ; *c1nc(C(F)(F)F)n2c1CN(*)CC2 ; *c1ccn2c1CN(*)CC2 ; *c1ccc2c(c1)N(*)CCN2C ; *c1ccc2c(c1)N(*)CCO2]

wherein the R²—Z^{2b}— substituent is attached to Ar² at any Ar² atom capable of being substituted.

In certain embodiments in which the Bcl-xL inhibitor is a compound of formula (IIa.1), Ar² is selected from

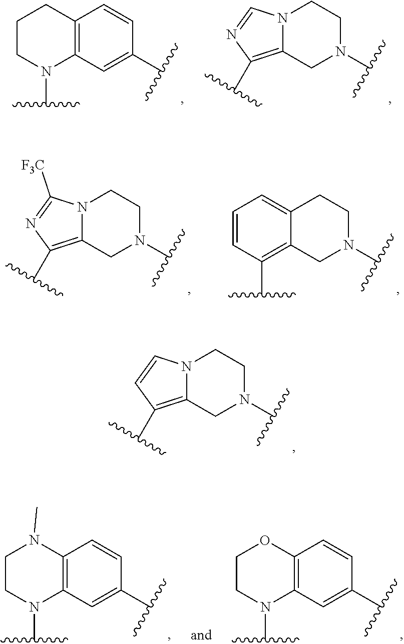

wherein the R¹²—Z^{2b}— substituent is attached to Ar² at any Ar² atom capable of being substituted.

In particular embodiments in which the Bcl-xL inhibitor is a compound of formula (IIa.1), Ar² is

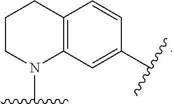 [SMILES: *c1ccc2c(c1)N(*)CCC2]

In particular embodiments in which the Bcl-xL inhibitor is a compound of formula (IIa.1), Ar² is

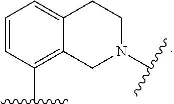 [SMILES: *c1cccc2c1CN(*)CC2]

In certain embodiments in which the Bcl-xL inhibitor is a compound of formula (IIa.1), Z^{2b}-R¹² is selected from H, F, CN, OCH₃, OH, NH₂, OCH₂CH₂OCH₃, N(CH₃)C(═O)CH₃, CH₂N(CH₃)C(═O)CH₃SCH₃, C(═O)N(CH₃)₂ and OCH₂CH₂N(CH₃)(C(═O)CH₃). In particular embodiments, Z^{2b}-R¹² is selected from H, F and CN. In particular embodiments, Z^{2b}-R¹² is H.

In embodiments where Z^{2b}—R¹² is substituted with hydroxyl (OH), the oxygen can serve as the point of attachment to a linking group (See Section 4.4.1.1).

In certain embodiments in which the Bcl-xL inhibitor is a compound of formula (IIa.1), Ar¹ is

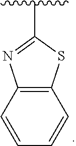 [SMILES: *c1nc2ccccc2s1]

In certain embodiments in which the Bcl-xL inhibitor is a compound of formula (IIa.1), the group

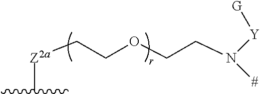

bonded to the adamantane ring is selected from:

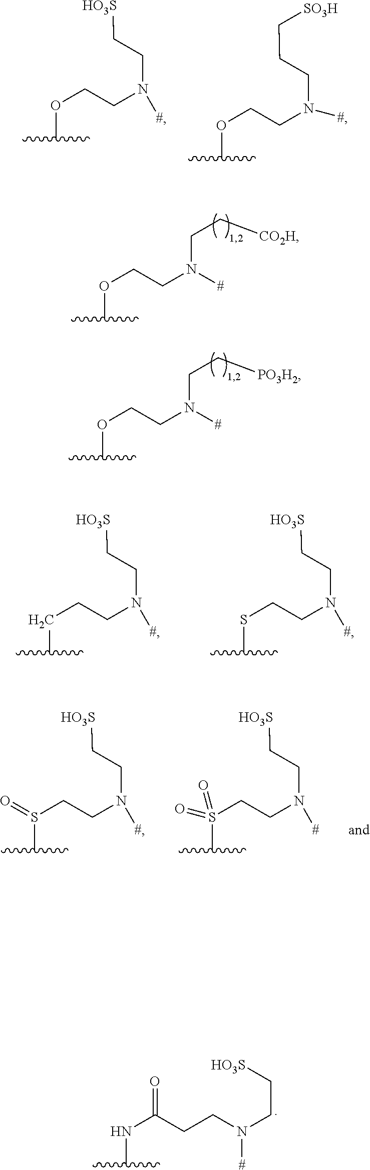

In certain embodiments, a compound of formula (IIa.1) may be converted into the compound of formula IIa.1.1, wherein n is selected from 1-3:

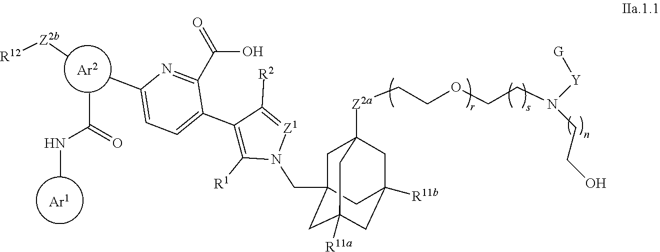

In certain embodiments, the compound of formula IIa.1.1 can be converted into a compound of formula IIa.1.2, wherein L represents a linker and LK represents a linkage formed between a reactive functional group on linker L and a complementary functional group on antibody.

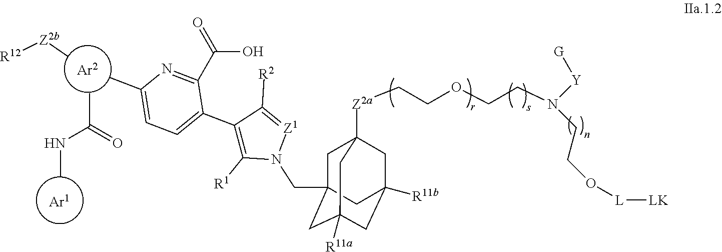

In certain embodiments in which the Bcl-xL inhibitor is a compound of formula (IIa), the compound has the structural formula (IIa.2),

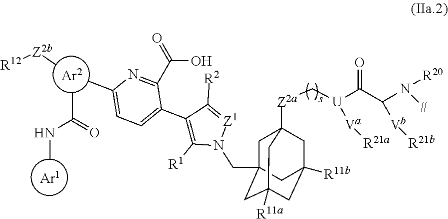

or salts thereof, wherein:
Ar¹, Ar², Z¹, Z^{2a}, Z^{2b}, R¹, R², R^{11a}, R^{11b}, R¹² and #are defined as above;
U is selected from N, O and CH, with the proviso that when U is O, then V^{a} and R^{21a} are absent;
R²⁰ is selected from H and C₁-C₄ alkyl;
R^{21a} and R^{21b} are each, independently from one another, absent or selected from H, C₁-C₄ alkyl and G, where G is selected from a polyol, PEG4-30, a salt and a moiety that is charged at physiological pH;
V^{a} and V^{b} are each, independently from one another, absent or selected from a bond, and an optionally substituted alkylene;
R²⁰ is selected from H and C₁-C₄ alkyl; and
s is 1, 2 or 3.

In certain embodiments in which the Bcl-xL inhibitor is a compound of formula (IIa.2), s is 2.

In certain embodiments in which the Bcl-xL inhibitor is a compound of formula (IIa.2), Z^{2a} is selected from O, NH, CH₂ and S. In particular embodiments, Z^{2a} is O. In certain embodiments, Z^{2a} of formula (IIa.2) is CR^{6a}R^{6b}. In certain embodiments, Z^{2a} of formula (IIa.2) is CH₂. In certain embodiments, Z^{2a} of formula (IIa.2) is S. In certain embodiments, Z^{2a} of formula (IIa.2) is —NR⁶C(O)—.

In certain embodiments in which the Bcl-xL inhibitor is a compound of formula (IIa.2), U is selected from N and O. In particular embodiments, U is O.

In certain embodiments in which the Bcl-xL inhibitor is a compound of formula (IIa.2), V^{a} is a bond, R^{21a} is a C₁-C₄ alkyl group, V^{b} is selected from methylene and ethylene and R^{21b} is G. In particular embodiments, V^{a} is a bond, R^{21a} is a methyl group and V^{b} is selected from methylene and ethylene and R^{21b} is G.

In certain embodiments in which the Bcl-xL inhibitor is a compound of formula (IIa.2), V^{a} is selected from methylene and ethylene, R^{21a} is G, V^{b} is selected from methylene and ethylene and R^{21b} is G. In particular embodiments, V^{a} is ethylene, R^{21a} is G, V^{b} is selected from methylene and ethylene and R^{21b} is G.

In certain embodiments in which the Bcl-xL inhibitor is a compound of formula (IIa.2), G is selected from

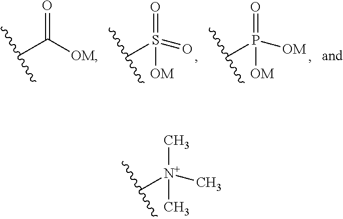

wherein M is hydrogen or a positively charged counterion. In particular embodiments, G is

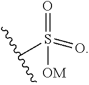

In particular embodiments, G is SO₃H.

In certain embodiments in which the Bcl-xL inhibitor is a compound of formula (IIa.2), R²⁰ is selected from hydrogen and a methyl group.

In certain embodiments in which the Bcl-xL inhibitor is a compound of formula (IIa.2), Ar² is selected from

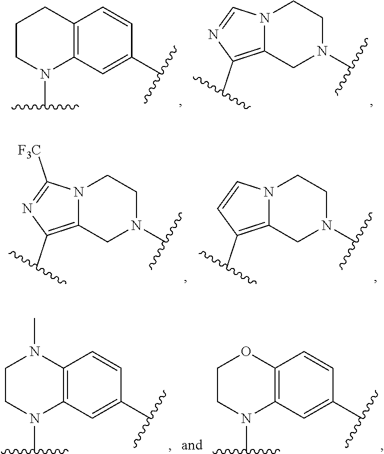

wherein the R¹²—Z^{2b}— substituent is attached to Ar² at any Ar² atom capable of being substituted.

In certain embodiments in which the Bcl-xL inhibitor is a compound of formula (IIa.2), Ar² is selected from

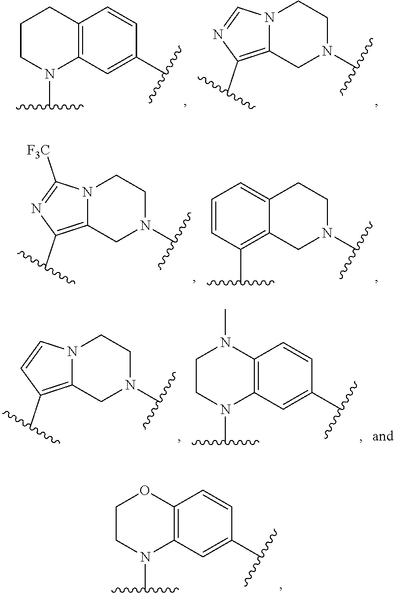

wherein the R¹²—Z^{2b}— substituent is attached to Ar² at any Ar² atom capable of being substituted.

In articular embodiments in which the Bcl-xL inhibitor is a compound of formula (IIa.2), Ar² is

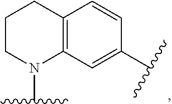 [SMILES: *c1ccc2c(c1)N(*)CCC2]

wherein the R¹²—Z^{2b}— substituent is attached to Ar² at any Ar² atom capable of being substituted.

In certain embodiments in which the Bcl-xL inhibitor is a compound of formula (IIa.2), Z^{2b}—R¹² is selected from H, F, CN, OCH₃, OH, NH₂, OCH₂CH₂OCH₃, N(CH₃)C(═O)CH₃, CH₂N(CH₃)C(═O)CH₃SCH₃, C(═O)N(CH₃)₂ and OCH₂CH₂N(CH₃)(C(═O)CH₃). In particular embodiments, Z^{2b}-R¹² is selected from H, F and CN. In particular embodiments, Z^{2b}—R¹² is H. In certain embodiments in which the Bcl-xL inhibitor is a compound of formula (IIa.2), Ar¹ is

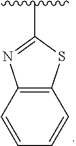 [SMILES: *c1nc2ccccc2s1]

In articular embodiments in which the Bcl-xL inhibitor is a compound of formula (IIa.2), Ar² is

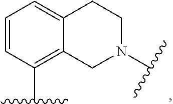 [SMILES: *c1cccc2c1CN(*)CC2]

wherein the R¹²—Z^{2b}— substituent is attached to Ar² at any Ar² atom capable of being substituted.

In certain embodiments in which the Bcl-xL inhibitor is a compound of formula (IIa), the compound has the structural formula (IIa.3),

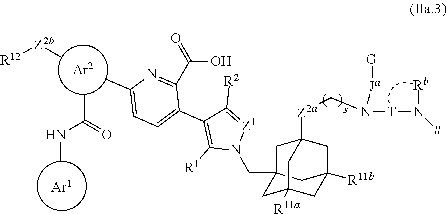

or salts thereof, wherein:
Ar¹, Ar², Z¹, Z^{2a}, Z^{2b}, R¹, R², R^{11a}, R^{11b}, R¹² and #are defined as above;
R^{b} is selected from H, C₁-C₄ alkyl and J^{b}-G or is optionally taken together with an atom of T to form a ring having between 3 and 7 atoms;
J^{a} and J^{b} are each, independently from one another, selected from optionally substituted alkylene and optionally substituted phenylene;
T is selected from optionally substituted alkylene, CH₂CH₂OCH₂CH₂OCH₂CH₂, CH₂CH₂OCH₂CH₂OCH₂CH₂OCH₂ and a polyethylene glycol containing from 4 to 10 ethylene glycol units;
G is selected from a polyol, PEG4-30, a salt and a moiety that is charged at physiological pH; and
s is 1, 2 or 3.

In certain embodiments in which the Bcl-xL inhibitor is a compound of formula (IIa.3), s is 1. In certain embodiments in which the Bcl-xL inhibitor is a compound of formula (IIa.3), s is 2.

In certain embodiments in which the Bcl-xL inhibitor is a compound of formula (IIa.3), Z^{2a} is selected from O, CH₂ and S. In particular embodiments, Z^{2a} is O. In certain embodiments, Z^{2a} of formula (IIa.3) is CR^{6a}R^{6b}. In certain embodiments, Z^{2a} of formula (IIa.3) is CH₂. In certain embodiments, Z^{2a} of formula (IIa.3) is S. In certain embodiments, Z^{2a} of formula (IIa.3) is —NR⁶C(O)—.

In certain embodiments in which the Bcl-xL inhibitor is a compound of formula (IIa.3), J^{a} is selected from methylene and ethylene and R^{b} is J^{b}-G, wherein J is methylene or ethylene. In some such embodiments, T is ethylene. In other such embodiments, T is CH₂CH₂OCH₂CH₂OCH₂CH₂. In other such embodiments, T is a polyethylene glycol containing from 4 to 10 ethylene glycol units.

In certain embodiments in which the Bcl-xL inhibitor is a compound of formula (IIa.3), J^{a} is selected from methylene and ethylene and R^{b} is taken together with an atom of T to form a ring having 4-6 ring atoms.

In certain embodiments in which the Bcl-xL inhibitor is a compound of formula (IIa.3), J^{a} is selected from methylene and ethylene and R^{b} is H or alkyl. In some such embodiments, T is ethylene. In other such embodiments, T is CH₂CH₂OCH₂CH₂OCH₂CH₂.

In certain embodiments in which the Bcl-xL inhibitor is a compound of formula (IIa.3), G is selected from

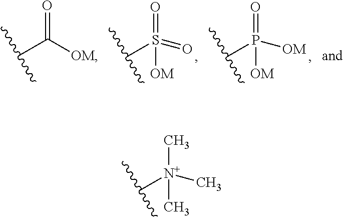

wherein M is hydrogen or a positively charged counterion. In particular embodiments, G is

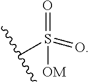

In particular embodiments, G is SO₃H.

In certain embodiments in which the Bcl-xL inhibitor is a compound of formula (IIa.3), R²⁰ is selected from hydrogen and a methyl group.

In certain embodiments in which the Bcl-xL inhibitor is a compound of formula (IIa.3), Ar² is selected from

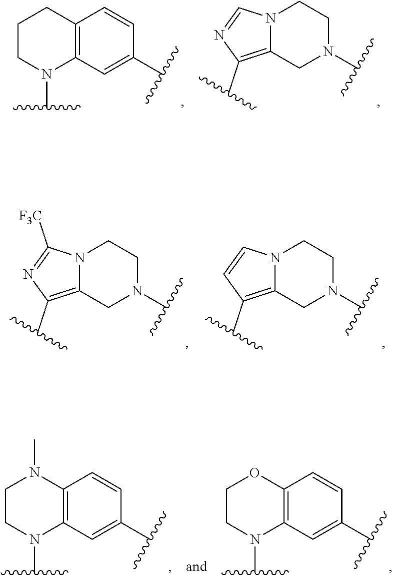 [SMILES: *c1ccc2c(c1)N(*)CCC2 ; *c1ncn2c1CN(*)CC2 ; *c1nc(C(F)(F)F)n2c1CN(*)CC2 ; *c1ccn2c1CN(*)CC2 ; *c1ccc2c(c1)N(*)CCN2C ; *c1ccc2c(c1)N(*)CCO2]

wherein the R¹²—Z^{2b}— substituent is attached to Ar² at any Ar² atom capable of being substituted.

In particular embodiments in which the Bcl-xL inhibitor is a compound of formula (IIa.3), Ar² is

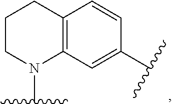 [SMILES: *c1ccc2c(c1)N(*)CCC2]

wherein the R¹²—Z^{2b}— substituent is attached to Ar² at any Ar² atom capable of being substituted. In certain embodiments in which the Bcl-xL inhibitor is a compound of formula (IIa.3), Ar² is selected from

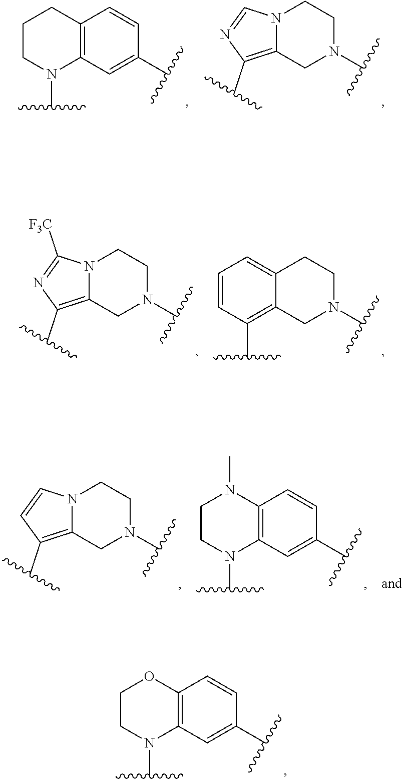

wherein the R¹²—Z^{2b}— substituent is attached to Ar² at any Ar² atom capable of being substituted. In particular embodiments in which the Bcl-xL inhibitor is a compound of formula (IIa.3), Ar² is

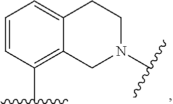 [SMILES: *c1cccc2c1CN(*)CC2]

wherein the R¹²—Z^{2b}— substituent is attached to Ar² at any Ar² atom capable of being substituted.

In certain embodiments in which the Bcl-xL inhibitor is a compound of formula (IIa.3), Z^{2b}-R¹² is selected from H, F, CN, OCH₃, OH, NH₂, OCH₂CH₂OCH₃, N(CH₃)C(═O)CH₃, CH₂N(CH₃)C(═O)CH₃SCH₃, C(═O)N(CH₃)₂ and OCH₂CH₂N(CH₃)(C(═O)CH₃). In particular embodiments, Z^{2b}-R¹² is selected from H, F and CN. In particular embodiments, Z^{2b}—R¹² is H.

In certain embodiments in which the Bcl-xL inhibitor is a compound of formula (IIa.3), Ar¹ is

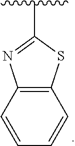 [SMILES: *c1nc2ccccc2s1]

In certain embodiments in which the Bcl-xL inhibitor is a compound of formula (IIa.3), the group

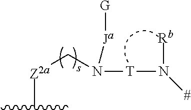

is selected from:

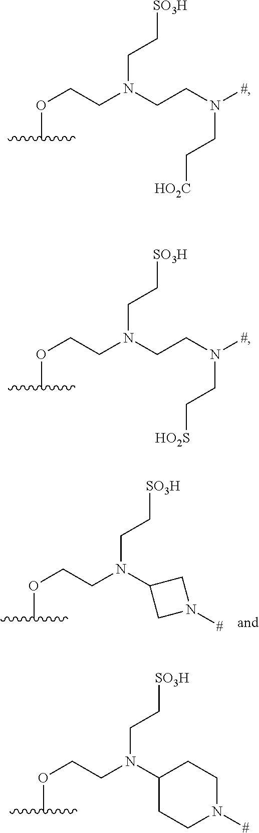

In certain embodiments in which the Bcl-xL inhibitor is a compound of formula (IIa.3), the group

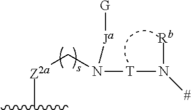

is selected from:

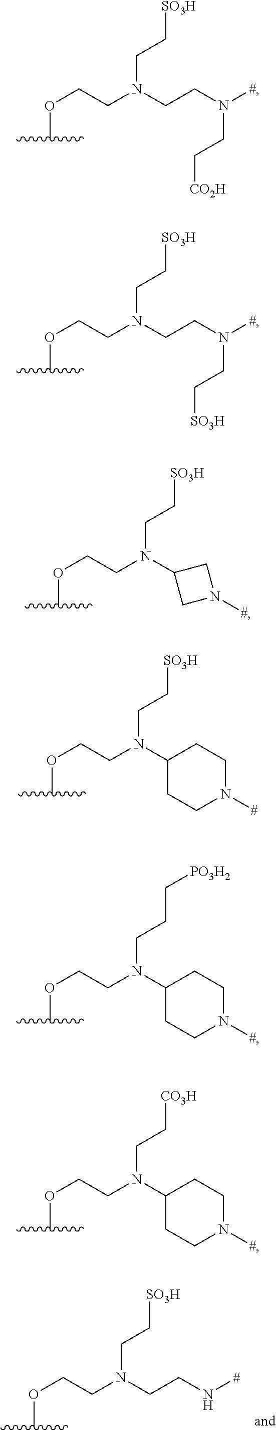

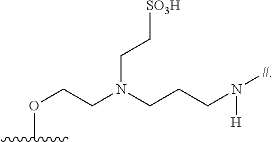

In certain embodiments the Bcl-xL inhibitor is a compound of formula (IIb). In certain embodiments in which the Bcl-xL inhibitor is a compound of formula (IIb), the compound has the structural formula (IIb.1),

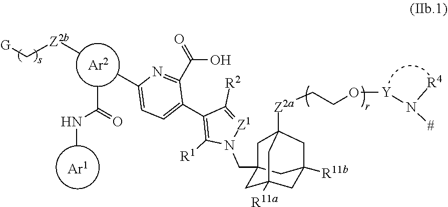

or salts thereof, wherein:
Ar¹, Ar², Z¹, Z^{2a}, Z^{2b}, R¹, R², R⁴, R^{11a}, R^{11b} and #are defined as above;
Y is optionally substituted alkylene;
G is selected from a polyol, PEG4-30, a salt and a moiety that is charged at physiological pH;
r is 0 or 1; and
s is 1, 2 or 3.

In certain embodiments in which the Bcl-xL inhibitor is a compound of formula (IIb.1), s is 1. In certain embodiments in which the Bcl-xL inhibitor is a compound of formula (IIb.1), s is 2. In certain embodiments in which the Bcl-xL inhibitor is a compound of formula (IIb.1), s is 3.

In certain embodiments in which the Bcl-xL inhibitor is a compound of formula (IIb.1), Z^{2a} is selected from O, CH₂, NH and S. In particular embodiments, Z^{2a} is O. In certain embodiments, Z^{2a} of formula (IIb.1) is CR^{6a}R^{6b}. In certain embodiments, Z^{2a} of formula (IIb.1) is CH₂. In certain embodiments, Z^{2a} of formula (IIb.1) is S. In certain embodiments, Z^{2a} of formula (IIb.1) is —NR⁶C(O)—.

In certain embodiments in which the Bcl-xL inhibitor is a compound of formula (IIb.1), Z^{2b} is selected from O, CH₂, NH, NCH₃ and S. In particular embodiments, Z^{2b} is O. In particular embodiments, Z^{2b} is NH. In particular embodiments, Z^{2b} is NCH₃.

In certain embodiments in which the Bcl-xL inhibitor is a compound of formula (IIb.1), Y is ethylene and r is 0.

In certain embodiments in which the Bcl-xL inhibitor is a compound of formula (IIb.1), Y is ethylene and r is 1.

In certain embodiments in which the Bcl-xL inhibitor is a compound of formula (IIb.1), R is H or methyl. In particular embodiments, R⁴ is methyl. In other embodiments, R⁴ is H.

In certain embodiments in which the Bcl-xL inhibitor is a compound of formula (IIb.1), R⁴ is taken together with an atom of Y to form a ring having 4-6 ring atoms. In particular embodiments, the ring is a cyclobutane ring. In other embodiments, the ring is a piperazine ring. In other embodiments, the ring is a morpholine ring.

In certain embodiments in which the Bcl-xL inhibitor is a compound of formula (IIb.1), G is selected from

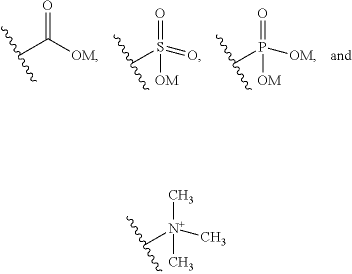

wherein M is hydrogen or a positively charged counterion. In particular embodiments, G is

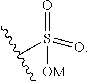

In other embodiments, G is SO₃H. In particular embodiments, G is NH₂. In other embodiments, G is PO₃H₂. In particular embodiments, G is NH₂. In particular embodiments, G is C(O)OH. In particular embodiments, G is polyol.

In certain embodiments in which the Bcl-xL inhibitor is a compound of formula (IIb.1), Ar² is selected from

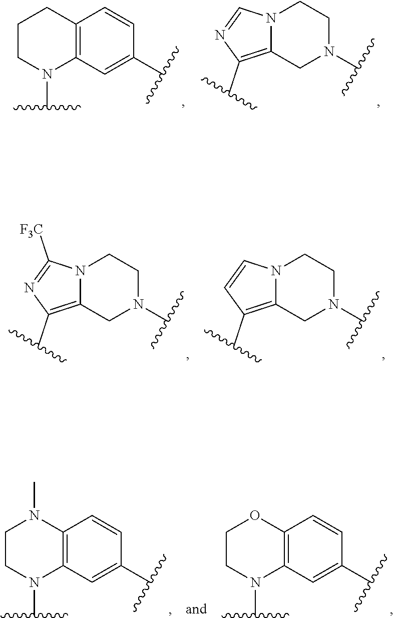 [SMILES: *c1ccc2c(c1)N(*)CCC2 ; *c1ncn2c1CN(*)CC2 ; *c1nc(C(F)(F)F)n2c1CN(*)CC2 ; *c1ccn2c1CN(*)CC2 ; *c1ccc2c(c1)N(*)CCN2C ; *c1ccc2c(c1)N(*)CCO2]

wherein the G-(CH₂)ₛ—Z^{2b}— substituent is attached to Ar² at any Ar² atom capable of being substituted.

In particular embodiments in which the Bcl-xL inhibitor is a compound of formula (IIb.1), Ar² is

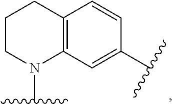 [SMILES: *c1ccc2c(c1)N(*)CCC2]

wherein the G-(CH₂)ₛ—Z^{2b}— substituent is attached to Ar² at any Ar² atom capable of being substituted. In certain embodiments in which the Bcl-xL inhibitor is a compound of formula (IIb.1), Ar² is selected from

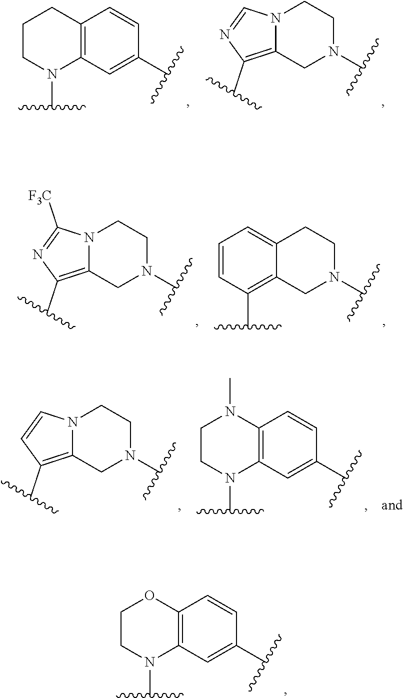

wherein the G-(CH₂)ₛ—Z^{2b}— substituent is attached to Ar² at any Ar² atom capable of being substituted. In particular embodiments in which the Bcl-xL inhibitor is a compound of formula (IIb.1), Ar² is

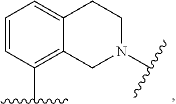 [SMILES: *c1cccc2c1CN(*)CC2]

wherein the G-(CH₂)ₛ—Z^{2b}— substituent is attached to Ar² at any Ar² atom capable of being substituted.

In certain embodiments in which the Bcl-xL inhibitor is a compound of formula (IIb.1), Ar¹ is

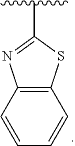 [SMILES: *c1nc2ccccc2s1]

In certain embodiments in which the Bcl-xL inhibitor is a compound of formula (IIb.1), the group

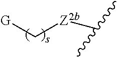

is selected from:

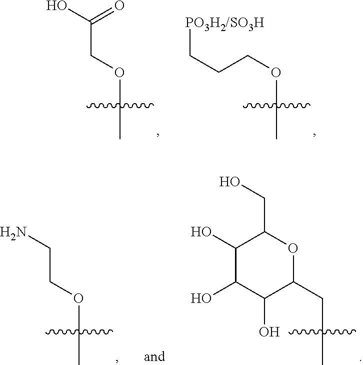

In certain embodiments in which the Bcl-xL inhibitor is a compound of formula (IIb.1), the group

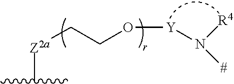

is selected from:

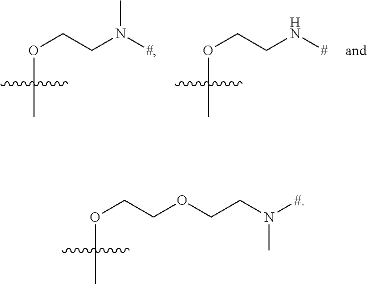

In certain embodiments in which the Bcl-xL inhibitor is a compound of formula (IIb.1), the group

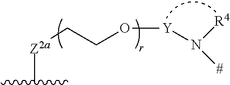

is selected from:

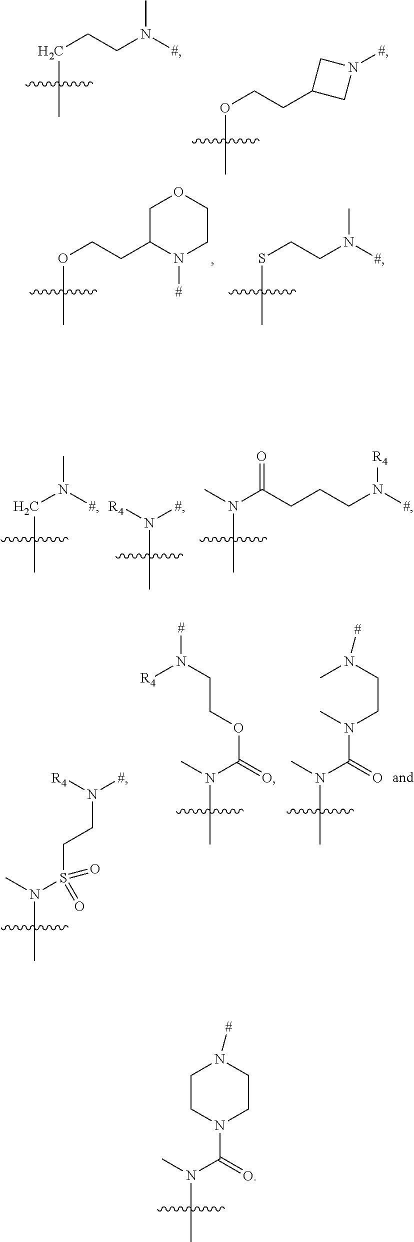

In certain embodiments the Bcl-xL inhibitor is a compound of formula (IIc). In certain embodiments in which the Bcl-xL inhibitor is a compound of formula (IIc), the compound has the structural formula (IIc.1)

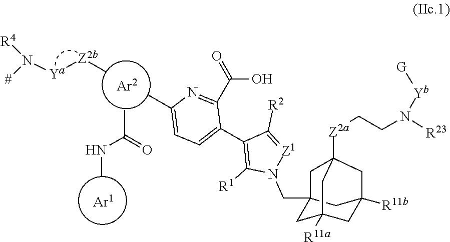

or salts thereof, wherein:
Ar¹, Ar², Z¹, Z^{2a}, Z^{2b}, R¹, R², R⁴, R^{11a}, R^{11b} and #are defined as above;
Y^{a} is optionally substituted alkylene;
Y^{b} is optionally substituted alkylene;
R² is selected from H and C₁-C₄ alkyl; and
G is selected from a polyol, PEG4-30, a salt and a moiety that is charged at physiological pH;

In certain embodiments in which the Bcl-xL inhibitor is a compound of formula (IIc.1), Z^{2a} is selected from O, CH₂, NH and S. In particular embodiments, Z^{2a} is O. In certain embodiments, Z^{2a} of formula (IIc.1) is CR^{6a}R^{6b}. In certain embodiments, Z^{2a} of formula (IIc.1) is S. In certain embodiments, Z^{2a} of formula (IIc.1) is —NR⁶C(O)—.

In certain embodiments in which the Bcl-xL inhibitor is a compound of formula (IIc.1), Z^{2b} is selected from O, CH₂, NH, NCH₃ and S. In particular embodiments, Z^{2b} is O. In particular embodiments, Z^{2b} is NH. In particular embodiments, Z^{2b} is NCH₃.

In certain embodiments in which the Bcl-xL inhibitor is a compound of formula (IIc.1), Z^{2b} is a bond. In some such embodiments Y^{a} is methylene or ethylene.

In certain embodiments in which the Bcl-xL inhibitor is a compound of formula (IIc.1), Z^{2b} is O. In some such embodiments Y^{a} is methylene, ethylene, or propylene.

In certain embodiments in which the Bcl-xL inhibitor is a compound of formula (IIc.1), Z^{2b} is NR⁶, where R⁶ is defined as above. In some such embodiments, R⁶ is taken together with an atom from Y^{a} to form a cycloalkyl or heterocyclyl ring having between 3 and 7 ring atoms. In some such embodiments, the ring has 5 atoms.

In certain embodiments in which the Bcl-xL inhibitor is a compound of formula (IIc.1), Y^{a} is ethylene.

In certain embodiments in which the Bcl-xL inhibitor is a compound of formula (IIc.1), Y^{a} is methylene.

In certain embodiments in which the Bcl-xL inhibitor is a compound of formula (IIc.1), Y^{a} is propylene.

In certain embodiments in which the Bcl-xL inhibitor is a compound of formula (IIc.1), R⁴ is H or methyl. In particular embodiments, R is H.

In certain embodiments in which the Bcl-xL inhibitor is a compound of formula (IIc.1), Y^{b} is ethylene or propylene. In particular embodiments, Y^{b} is ethylene.

In certain embodiments in which the Bcl-xL inhibitor is a compound of formula (IIc.1), R²³ is methyl.

In certain embodiments in which the Bcl-xL inhibitor is a compound of formula (IIc.1), R²³ is H.

In certain embodiments in which the Bcl-xL inhibitor is a compound of formula (IIc.1), G is selected from

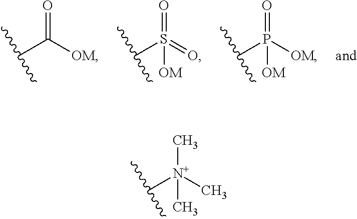

wherein M is hydrogen or a positively charged counterion. In particular embodiments, G is

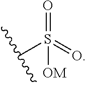

In particular embodiments, G is SO₃H.

In certain embodiments in which the Bcl-xL inhibitor is a compound of formula (IIc.1), Ar² is selected from

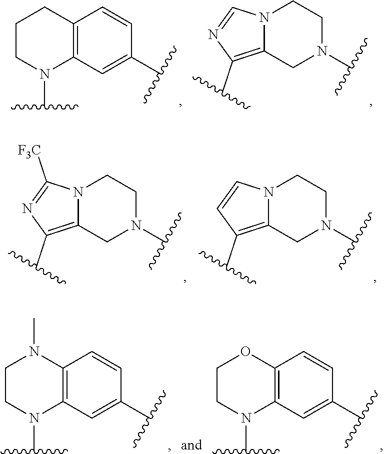

wherein the #—N(R⁴)—Y^{a}—Z^{2b}— substituent is attached to Ar² at any Ar² atom capable of being substituted.

In particular embodiments in which the Bcl-xL inhibitor is a compound of formula (IIc.1), Ar² is

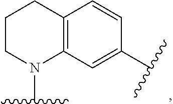 [SMILES: *c1ccc2c(c1)N(*)CCC2]

wherein the #—N(R⁴)—Y^{a}—Z^{2b}— substituent is attached to Ar² at any Ar² atom capable of being substituted. In certain embodiments in which the Bcl-xL inhibitor is a compound of formula (IIc.1), Ar² is selected from

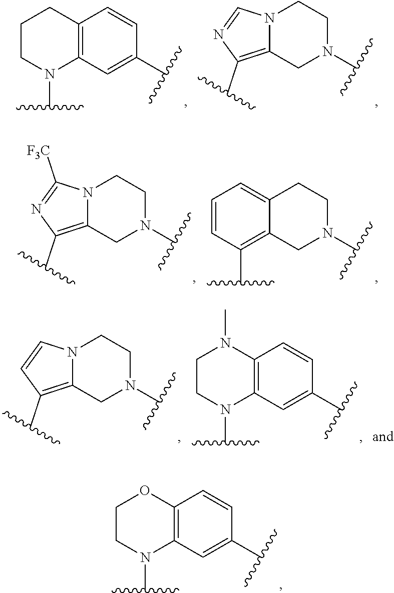

wherein the #—N(R⁴)—Y^{a}—Z^{2b}— substituent is attached to Ar² at any Ar² atom capable of being substituted. In particular embodiments in which the Bcl-xL inhibitor is a compound of formula (IIc.1), Ar² is

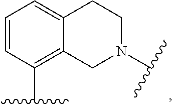 [SMILES: *c1cccc2c1CN(*)CC2]

wherein the #—N(R⁴)—Y^{a}—Z^{2b}— substituent is attached to Ar² at any Ar² atom capable of being substituted.

In certain embodiments in which the Bcl-xL inhibitor is a compound of formula (IIc.1), Ar¹ is

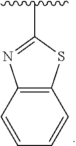 [SMILES: *c1nc2ccccc2s1]

In certain embodiments in which the Bcl-xL inhibitor is a compound of formula (IIc.1), the group

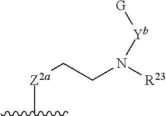

is selected from:

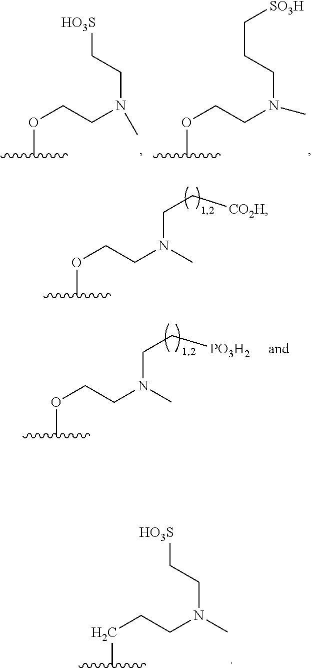

In other embodiments in which the Bcl-xL inhibitor is a compound of formula (IIc.1), the group

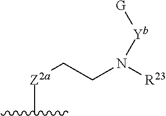

is selected from:

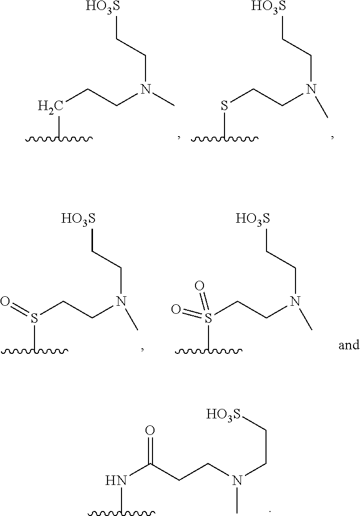

In certain embodiments in which the Bcl-xL inhibitor is a compound of formula (IIc), the compound has the structural formula (IIc.2),

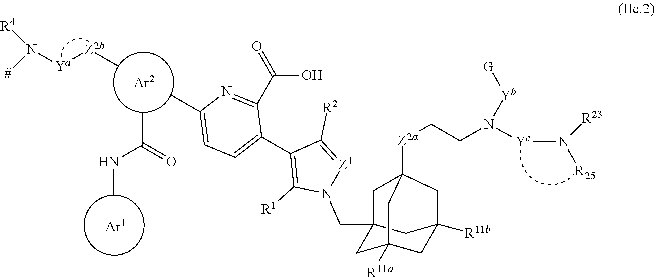

or salts thereof, wherein:
Ar¹, Ar², Z¹, Z^{2a}, Z^{2b}, R¹, R², R⁴, R^{11a}, R^{11b} and #are defined as above;
Y^{a} is optionally substituted alkylene;
Y^{b} is optionally substituted alkylene;
Y^{c} is optionally substituted alkylene;
R²³ is selected from H and C₁-C₄ alkyl;
R²⁵ is Y^{b}-G or is taken together with an atom of Y^{c} to form a ring having 4-6 ring atoms; and
G is selected from a polyol, PEG4-30, a salt and a moiety that is charged at physiological pH.

In certain embodiments in which the Bcl-xL inhibitor is a compound of formula (IIc.2), Z^{2a} is selected from O, CH₂, NH and S. In particular embodiments, Z^{2a} is O. In certain embodiments, Z^{2a} of formula (IIc.2) is CR^{6a}R^{6b}. In certain embodiments, Z^{2a} of formula (IIc.2) is S. In certain embodiments, Z^{2a} of formula (IIc.2) is —NR⁶C(O)—. In certain embodiments in which the Bcl-xL inhibitor is a compound of formula (IIc.2), Z^{2b} is selected from O, CH₂, NH, NCH₃ and S. In particular embodiments, Z^{2b} is O. In particular embodiments, Z^{2b} is NH. In particular embodiments, Z^{2b} is NCH₃.

In certain embodiments in which the Bcl-xL inhibitor is a compound of formula (IIc.2), Z^{2b} is a bond. In some such embodiments Y^{a} is methylene or ethylene.

In certain embodiments in which the Bcl-xL inhibitor is a compound of formula (IIc.2), Z^{2b} is NR⁶, where R⁶ is defined as above. In some such embodiments, R⁶ is taken together with an atom from Y^{a} to form a cycloalkyl or heterocyclyl ring having between 3 and 7 ring atoms. In some such embodiments, the ring has 5 atoms.

In certain embodiments in which the Bcl-xL inhibitor is a compound of formula (IIc.2), Y^{a} is ethylene.

In certain embodiments in which the Bcl-xL inhibitor is a compound of formula (IIc.2), Y^{a} is methylene.

In certain embodiments in which the Bcl-xL inhibitor is a compound of formula (IIc.2), R⁴ is H or methyl.

In certain embodiments in which the Bcl-xL inhibitor is a compound of formula (IIc.2), Y^{b} is ethylene or propylene. In particular embodiments, Y^{b} is ethylene.

In certain embodiments in which the Bcl-xL inhibitor is a compound of formula (IIc.2), Y^{c} is ethylene or propylene. In particular embodiments, Y^{b} is ethylene.

In certain embodiments in which the Bcl-xL inhibitor is a compound of formula (IIc.2), R²⁵ is taken together with an atom of Y^{c} to form a ring having 4 or 5 ring atoms.

In certain embodiments in which the Bcl-xL inhibitor is a compound of formula (IIc.2), R²³ is methyl.

In certain embodiments in which the Bcl-xL inhibitor is a compound of formula (IIc.2), G is selected from

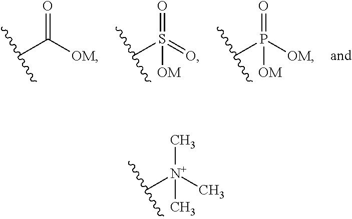

wherein M is hydrogen or a positively charged counterion. In particular embodiments, G is

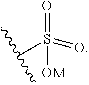

In particular embodiments, G is SO₃H.

In certain embodiments in which the Bcl-xL inhibitor is a compound of formula (IIc.2), Ar² is selected from

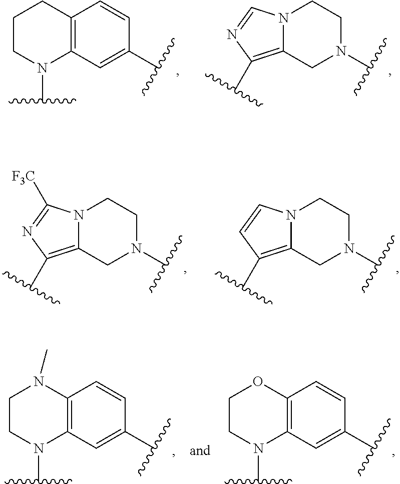 [SMILES: *c1ccc2c(c1)N(*)CCC2 ; *c1ncn2c1CN(*)CC2 ; *c1nc(C(F)(F)F)n2c1CN(*)CC2 ; *c1ccn2c1CN(*)CC2 ; *c1ccc2c(c1)N(*)CCN2C ; *c1ccc2c(c1)N(*)CCO2]

wherein the #—N(R⁴)—Y^{a}—Z^{2b}-substituent is attached to Ar² at any Ar² atom capable of being substituted.

In particular embodiments in which the Bcl-xL inhibitor is a compound of formula (IIc.2), Ar² is

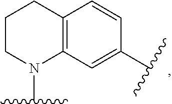 [SMILES: *c1ccc2c(c1)N(*)CCC2]

wherein the #—N(R⁴)—Y^{a}—Z^{2b}— substituent is attached to Ar² at any Ar² atom capable of being substituted. In certain embodiments in which the Bcl-xL inhibitor is a compound of formula (IIc.2), Ar² is selected from

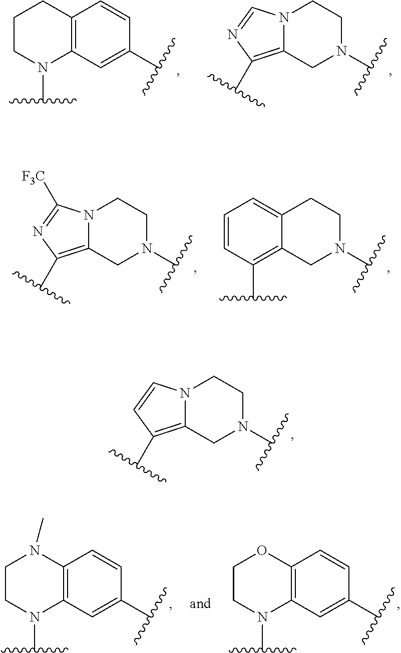

wherein the #—N(R⁴)—Y^{a}—Z^{2b}— substituent is attached to Ar² at any Ar² atom capable of being substituted. In articular embodiments in which the Bcl-xL inhibitor is a compound of formula (IIc.2), Ar² is

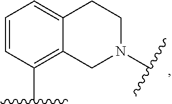 [SMILES: *c1cccc2c1CN(*)CC2]

wherein the #—N(R⁴)—Y^{a}—Z^{2b}— substituent is attached to Ar² at any Ar² atom capable of being substituted.

In certain embodiments in which the Bcl-xL inhibitor is a compound of formula (IIc.2), Ar¹ is

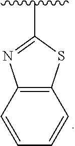 [SMILES: *c1nc2ccccc2s1]

In certain embodiments in which the Bcl-xL inhibitor is a compound of formula (IIc.2), the group

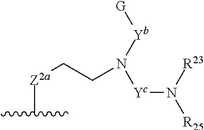

is selected from:

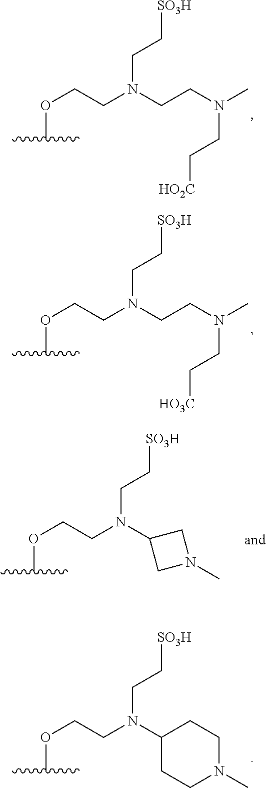

In certain embodiments in which the Bcl-xL inhibitor is a compound of formula (IId), the compound has the structural formula (IId.1),

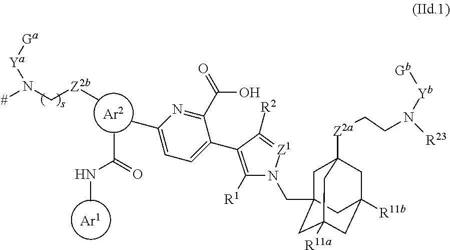

or salts thereof, wherein:
Ar¹, Ar², Z¹, Z^{2a}, Z^{2b}, R¹, R², R^{11a}, R^{11b} and #are defined as above;
Y^{a} is optionally substituted alkylene;
Y^{b} is optionally substituted alkylene;
R²³ is selected from H and C₁-C₄ alkyl;
G^{a} is selected from a polyol, PEG4-30, a salt and a moiety that is charged at physiological pH;
G^{b} is selected from a polyol, PEG4-30, a salt and a moiety that is charged at physiological pH;

In certain embodiments in which the Bcl-xL inhibitor is a compound of formula (IId.1), s is 1.

In certain embodiments in which the Bcl-xL inhibitor is a compound of formula (IId.1), s is 2.

In certain embodiments in which the Bcl-xL inhibitor is a compound of formula (IId.1), Z^{2a} is selected from O, NH, CH₂ and S. In particular embodiments, Z^{2a} is O. In certain embodiments, Z^{2a} of formula (IId.1) is CR^{6a}R^{6b}. In certain embodiments, Z^{2a} of formula (IId.1) is S. In certain embodiments, Z^{2a} of formula (IId.1) is —NR⁶C(O)—.

In certain embodiments in which the Bcl-xL inhibitor is a compound of formula (IId.1), Z^{2b} is selected from O, NH, CH₂ and S. In particular embodiments, Z^{2b} is O.

In certain embodiments in which the Bcl-xL inhibitor is a compound of formula (IId.1), Y^{a} is selected from ethylene, propylene and butylene. In particular embodiments, Y is ethylene.

In certain embodiments in which the Bcl-xL inhibitor is a compound of formula (IId.1), Y^{a} is selected from ethylene, propylene and butylene. In particular embodiments, Y is ethylene.

In certain embodiments in which the Bcl-xL inhibitor is a compound of formula (IId.1), G^{a} is selected from

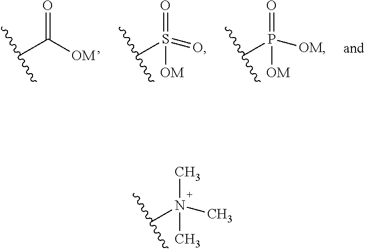

wherein M is hydrogen or a positively charged counterion. In particular embodiments, G^{a} is

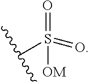

In particular embodiments, G^{a} is SO₃H. In particular embodiments, G^{a} is CO₂H.

In certain embodiments in which the Bcl-xL inhibitor is a compound of formula (IId.1), G^{b} is selected from

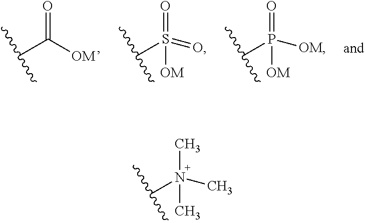

wherein M is hydrogen or a positively charged counterion. In particular embodiments, G^{b} is

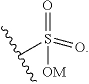

In particular embodiments, G^{b} is SO₃H. In particular embodiments, G^{b} is CO₂H.

In certain embodiments in which the Bcl-xL inhibitor is a compound of formula (IId.1), R²³ is methyl.

In certain embodiments in which the Bcl-xL inhibitor is a compound of formula (IId.1), Ar² is selected from

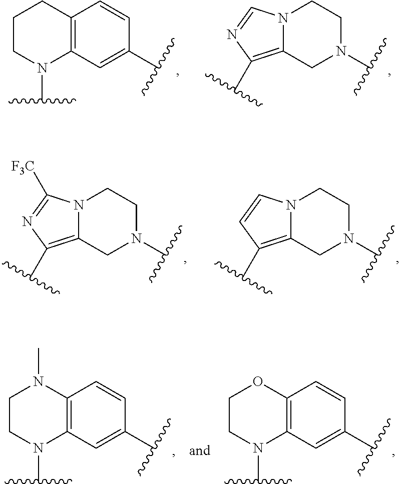 [SMILES: *c1ccc2c(c1)N(*)CCC2 ; *c1ncn2c1CN(*)CC2 ; *c1nc(C(F)(F)F)n2c1CN(*)CC2 ; *c1ccn2c1CN(*)CC2 ; *c1ccc2c(c1)N(*)CCN2C ; *c1ccc2c(c1)N(*)CCO2]

wherein the G^{a}-Y^{a}—N(#)—(CH₂)ₛ—Z^{2b}-substituent is attached to Ar² at any Ar² atom capable of being substituted.

In particular embodiments in which the Bcl-xL inhibitor is a compound of formula (IId.1), Ar² is

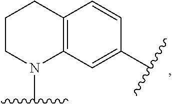 [SMILES: *c1ccc2c(c1)N(*)CCC2]

wherein the G^{a}-Y^{a}—N(#)—(CH₂)ₛ—Z^{2b}— substituent is attached to Ar² at any Ar² atom capable of being substituted. In certain embodiments in which the Bcl-xL inhibitor is a compound of formula (IId.1), Ar² is selected from

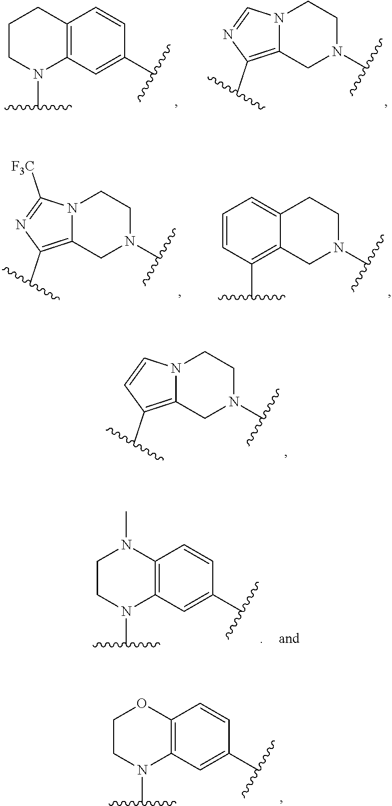

wherein the G^{a}-Y^{a}—N(#)—(CH₂)ₛ—Z^{2b}— substituent is attached to Ar² at any Ar² atom capable of being substituted. In particular embodiments in which the Bcl-xL inhibitor is a compound of formula (IId.1), Ar² is

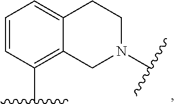 [SMILES: *c1cccc2c1CN(*)CC2]

wherein the G^{a}-Y^{a}—N(#)—(CH₂)ₛ—Z^{2b}— substituent is attached to Ar² at any Ar² atom capable of being substituted.

In certain embodiments in which the Bcl-xL inhibitor is a compound of formula (IId.1), Ar¹ is

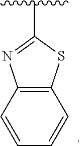 [SMILES: *c1nc2ccccc2s1]

In certain embodiments, R^{11a} and R^{11b} of formulae (IIa)-(IId) are the same. In a particular embodiment, R^{11a} and R^{11b} are each methyl.

In certain embodiments, the compounds of formulae (IIa)-(IId) include one of the following cores (C.1)-(C.21):

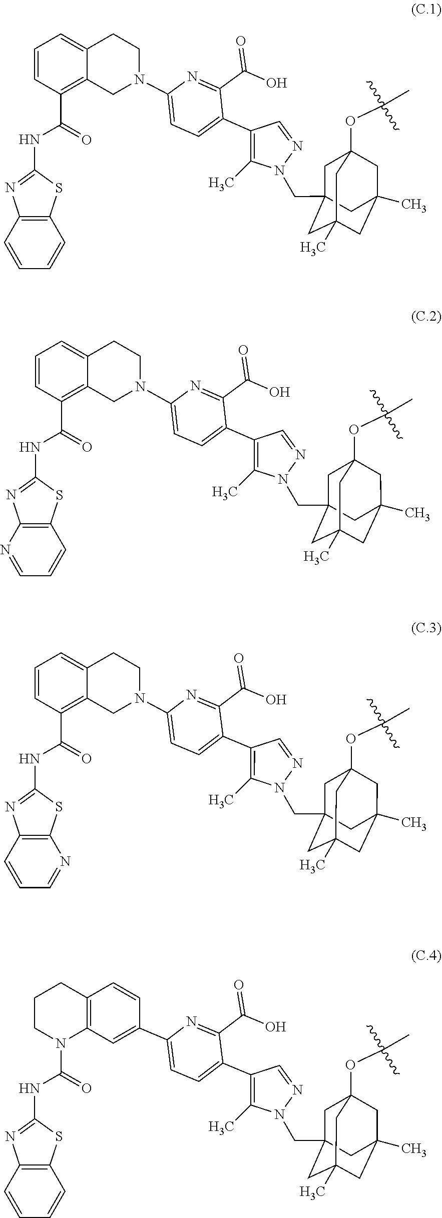 [SMILES: *OC12CC3(C)CC(C)(CC(Cn4ncc(-c5ccc(N6CCc7cccc(C(=O)Nc8nc9ccccc9s8)c7C6)nc5C(=O)O)c4C)(C3)C1)C2 ; *OC12CC3(C)CC(C)(CC(Cn4ncc(-c5ccc(N6CCc7cccc(C(=O)Nc8nc9ncccc9s8)c7C6)nc5C(=O)O)c4C)(C3)C1)C2 ; *OC12CC3(C)CC(C)(CC(Cn4ncc(-c5ccc(N6CCc7cccc(C(=O)Nc8nc9cccnc9s8)c7C6)nc5C(=O)O)c4C)(C3)C1)C2 ; *OC12CC3(C)CC(C)(CC(Cn4ncc(-c5ccc(-c6ccc7c(c6)N(C(=O)Nc6nc8ccccc8s6)CCC7)nc5C(=O)O)c4C)(C3)C1)C2]

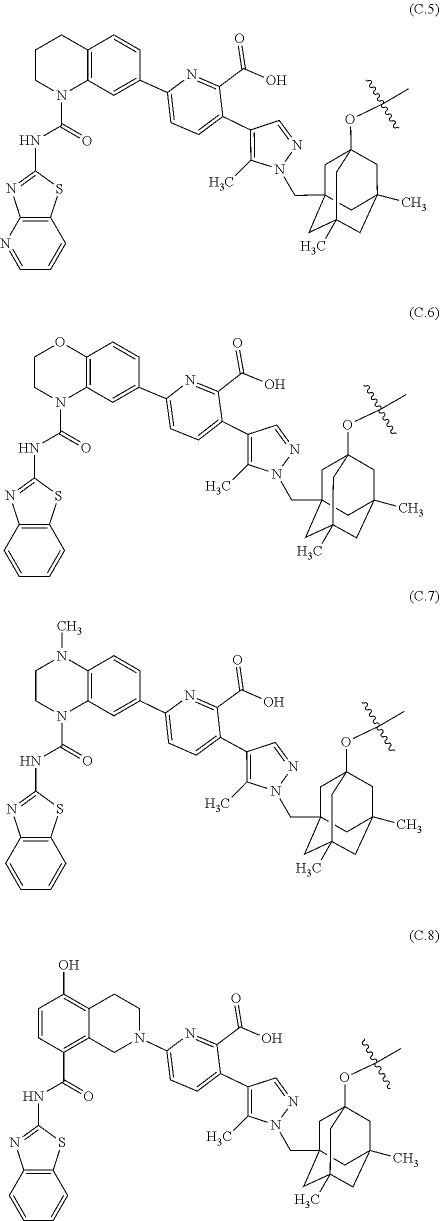 [SMILES: *OC12CC3(C)CC(C)(CC(Cn4ncc(-c5ccc(-c6ccc7c(c6)N(C(=O)Nc6nc8ncccc8s6)CCC7)nc5C(=O)O)c4C)(C3)C1)C2 ; *OC12CC3(C)CC(C)(CC(Cn4ncc(-c5ccc(-c6ccc7c(c6)N(C(=O)Nc6nc8ccccc8s6)CCO7)nc5C(=O)O)c4C)(C3)C1)C2 ; *OC12CC3(C)CC(C)(CC(Cn4ncc(-c5ccc(-c6ccc7c(c6)N(C(=O)Nc6nc8ccccc8s6)CCN7C)nc5C(=O)O)c4C)(C3)C1)C2 ; *OC12CC3(C)CC(C)(CC(Cn4ncc(-c5ccc(N6CCc7c(O)ccc(C(=O)Nc8nc9ccccc9s8)c7C6)nc5C(=O)O)c4C)(C3)C1)C2]

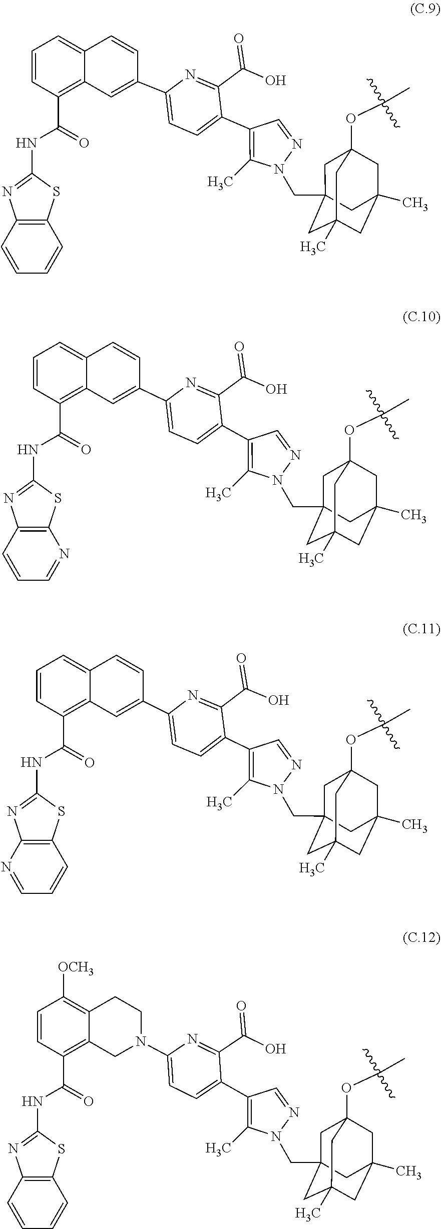 [SMILES: *OC12CC3(C)CC(C)(CC(Cn4ncc(-c5ccc(-c6ccc7cccc(C(=O)Nc8nc9ccccc9s8)c7c6)nc5C(=O)O)c4C)(C3)C1)C2 ; *OC12CC3(C)CC(C)(CC(Cn4ncc(-c5ccc(-c6ccc7cccc(C(=O)Nc8nc9cccnc9s8)c7c6)nc5C(=O)O)c4C)(C3)C1)C2 ; *OC12CC3(C)CC(C)(CC(Cn4ncc(-c5ccc(-c6ccc7cccc(C(=O)Nc8nc9ncccc9s8)c7c6)nc5C(=O)O)c4C)(C3)C1)C2 ; *OC12CC3(C)CC(C)(CC(Cn4ncc(-c5ccc(N6CCc7c(OC)ccc(C(=O)Nc8nc9ccccc9s8)c7C6)nc5C(=O)O)c4C)(C3)C1)C2]

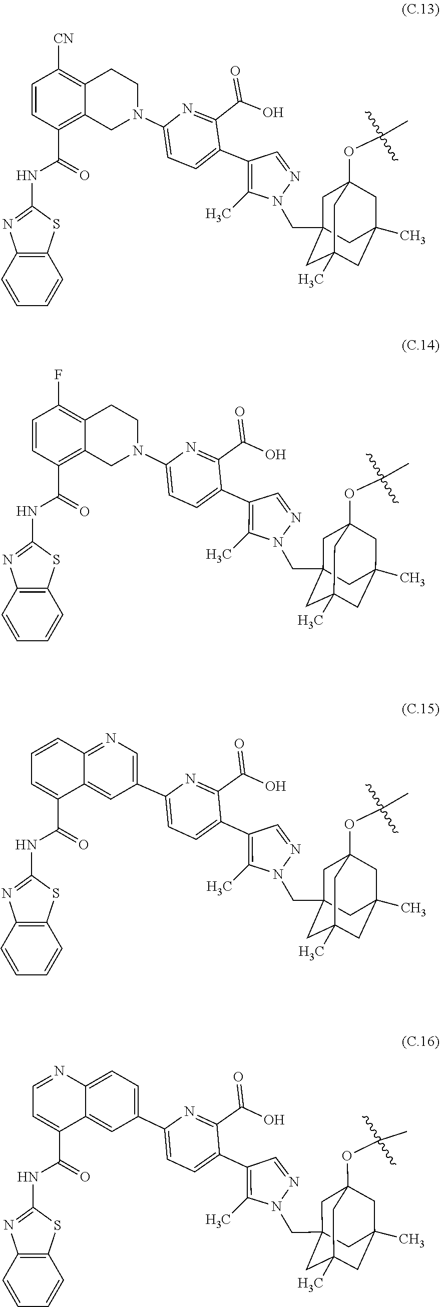 [SMILES: *OC12CC3(C)CC(C)(CC(Cn4ncc(-c5ccc(N6CCc7c(C#N)ccc(C(=O)Nc8nc9ccccc9s8)c7C6)nc5C(=O)O)c4C)(C3)C1)C2 ; *OC12CC3(C)CC(C)(CC(Cn4ncc(-c5ccc(N6CCc7c(F)ccc(C(=O)Nc8nc9ccccc9s8)c7C6)nc5C(=O)O)c4C)(C3)C1)C2 ; *OC12CC3(C)CC(C)(CC(Cn4ncc(-c5ccc(-c6cnc7cccc(C(=O)Nc8nc9ccccc9s8)c7c6)nc5C(=O)O)c4C)(C3)C1)C2 ; *OC12CC3(C)CC(C)(CC(Cn4ncc(-c5ccc(-c6ccc7nccc(C(=O)Nc8nc9ccccc9s8)c7c6)nc5C(=O)O)c4C)(C3)C1)C2]

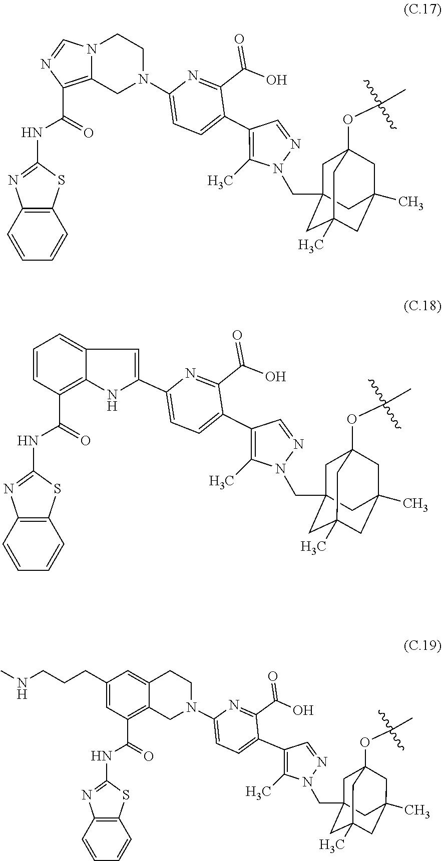

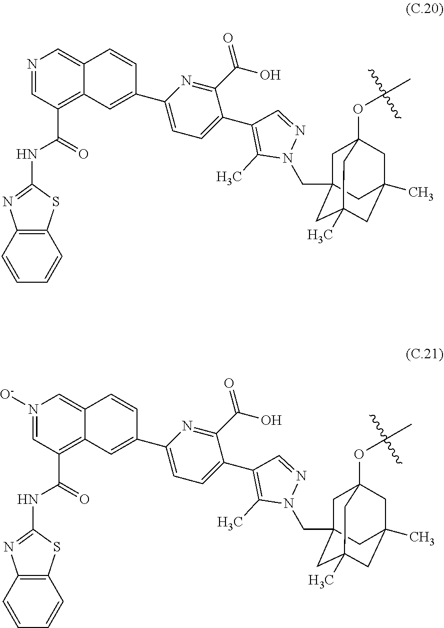

Exemplary Bcl-xL inhibitors according to structural formulae (IIa)-(IId) that may be used in the methods described herein in unconjugated form and/or included in the ADCs described herein include the following compounds, and/or salts thereof:

| App | Bcl-xL |
|---|---|
| Ex. No. | Inhibitor Cmpd No |
| 1.1 | W2.01 |
| 1.2 | W2.02 |
| 1.3 | W2.03 |
| 1.5 | W2.05 |
| 1.6 | W2.06 |
| 1.7 | W2.07 |
| 1.8 | W2.08 |
| 1.9 | W2.09 |
| 1.10 | W2.10 |
| 1.11 | W2.11 |
| 1.12 | W2.12 |
| 1.13 | W2.13 |
| 1.14 | W2.14 |
| 1.15 | W2.15 |
| 1.16 | W2.16 |
| 1.17 | W2.17 |
| 1.18 | W2.18 |
| 1.19 | W2.19 |
| 1.20 | W2.20 |
| 1.21 | W2.21 |
| 1.22 | W2.22 |
| 1.23 | W2.23 |
| 1.24 | W2.24 |
| 1.25 | W2.25 |
| 1.26 | W2.26 |
| 1.27 | W2.27 |
| 1.28 | W2.28 |
| 1.29 | W2.29 |
| 1.30 | W2.30 |
| 1.31 | W2.31 |
| 1.32 | W2.32 |
| 1.33 | W2.33 |
| 1.34 | W2.34 |
| 1.35 | W2.35 |
| 1.36 | W2.36 |
| 1.37 | W2.37 |
| 1.38 | W2.38 |
| 1.39 | W2.39 |
| 1.40 | W2.40 |
| 1.41 | W2.41 |
| 1.42 | W2.42 |
| 1.43 | W2.43 |
| 1.44 | W2.44 |
| 1.45 | W2.45 |
| 1.46 | W2.46 |
| 1.47 | W2.47 |
| 1.48 | W2.48 |
| 1.49 | W2.49 |
| 1.50 | W2.50 |
| 1.51 | W2.51 |
| 1.52 | W2.52 |
| 1.53 | W2.53 |
| 1.54 | W2.54 |
| 1.55 | W2.55 |
| 1.56 | W2.56 |
| 1.57 | W2.57 |
| 1.58 | W2.58 |
| 1.59 | W2.59 |
| 1.60 | W2.60 |
| 1.61 | W2.61 |
| 1.62 | W2.62 |
| 1.63 | W2.63 |
| 1.64 | W2.64 |
| 1.65 | W2.65 |
| 1.66 | W2.66 |
| 1.67 | W2.67 |
| 1.68 | W2.68 |
| 1.69 | W2.69 |
| 1.70 | W2.70 |
| 1.71 | W2.71 |
| 1.72 | W2.72 |
| 1.73 | W2.73 |
| 1.74 | W2.74 |
| 1.75 | W2.75 |
| 1.76 | W2.76 |
| 1.77 | W2.77 |
| 1.78 | W2.78 |
| 1.79 | W2.79 |
| 1.80 | W2.80 |
| 1.81 | W2.81 |
| 1.82 | W2.82 |
| 1.83 | W2.83 |
| 1.84 | W2.84 |
| 1.85 | W2.85 |
| 1.86 | W2.86 |
| 1.87 | W2.87 |
| 1.88 | W2.88 |
| 1.89 | W2.89 |
| 1.90 | W2.90 |
| 1.91 | W2.91 |

In certain embodiments, the Bcl-xL inhibitors according to structural formulae (IIa)-(IId) are selected from the group consisting of W2.01, W2.02, W2.03, W2.04, W2.05, W2.06, W2.07, W2.08, W2.09, W2.10, W2.11, W2.12, W2.13, W2.14, W2.15, W2.16, W2.17, W2.18, W2.19, W2.20, W2.21, W2.22, W2.23, W2.24, W2.25, W2.26, W2.27, W2.28, W2.29, W2.30, W2.31, W2.32, W2.33, W2.34, W2.35, W2.36, W2.37, W2.38, W2.39, W2.40, W2.41, W2.42, W2.43, W2.44, W2.45, W2.46, W2.47, W2.48, W2.49, W2.50, W2.51, W2.52, W2.53, W2.54, W2.55, W2.56, W2.57, W2.58, W2.59, W2.60, W2.61, W2.62, W2.63, W2.64, W2.65, W2.66, W2.67, W2.68, W2.69, W2.70, W2.71, W2.72, W2.73, W2.74, W2.75, W2.76, W2.77, W2.78, W2.79, W2.80, W2.81, W2.82, W2.83, W2.84, W2.85, W2.86, W2.87, W2.88, W2.89, W2.90, and W2.91, or pharmaceutically acceptable salts thereof.

In certain embodiments, the ADC, or a pharmaceutically acceptable salt thereof, comprises a drug linked to an antibody by way of a linker, wherein the drug is a Bcl-xL inhibitor selected from the group consisting of W2.01, W2.02, W2.03, W2.04, W2.05, W2.06, W2.07, W2.08, W2.09, W2.10, W2.11, W2.12, W2.13, W2.14, W2.15, W2.16, W2.17, W2.18, W2.19, W2.20, W2.21, W2.22, W2.23, W2.24, W2.25, W2.26, W2.27, W2.28, W2.29, W2.30, W2.31, W2.32, W2.33, W2.34, W2.35, W2.36, W2.37, W2.38, W2.39, W2.40, W2.41, W2.42, W2.43, W2.44, W2.45, W2.46, W2.47, W2.48, W2.49, W2.50, W2.51, W2.52, W2.53, W2.54, W2.55, W2.56, W2.57, W2.58, W2.59, W2.60, W2.61, W2.62, W2.63, W2.64, W2.65, W2.66, W2.67, W2.68, W2.69, W2.70, W2.71, W2.72, W2.73, W2.74, W2.75, W2.76, W2.77, W2.78, W2.79, W2.80, W2.81, W2.82, W2.83, W2.84, W2.85, W2.86, W2.87, W2.88, W2.89, W2.90, and W2.91.

The Bcl-xL inhibitors bind to and inhibit anti-apoptotic Bcl-xL proteins, inducing apoptosis. The ability of specific Bcl-xL inhibitors according to structural formulae (IIa)-(IId) to bind to and inhibit Bcl-xL activity may be confirmed in standard binding and activity assays, including, for example, the TR-FRET Bcl-xL binding assays described in Tao et al., 2014, ACS Med. Chem. Lett., 5:1088-1093. A specific TR-FRET Bcl-xL binding assay that can be used to confirm Bcl-xL binding is provided in Example 4, below. Typically, Bcl-xL inhibitors useful as inhibitors per se and in the ADCs described herein will exhibit a Kᵢ in the binding assay of Example 5 of less than about 1 nM, but may exhibit a significantly lower Kᵢ, for example a Kᵢ of less than about 1, 0.1, or even 0.01 nM.

Bcl-xL inhibitory activity may also be confirmed in standard cell-based cytotoxicity assays, such as the FL5.12 cellular and Molt4 cytotoxicity assays described in Tao et al., 2014, ACS Med. Chem. Lett., 5:1088-1093. A specific Molt-4 cellular cytotoxicity assay that may be used to confirm Bcl-xL inhibitory activity of specific Bcl-xL inhibitors that are able to permeate cell membranes is provided in Examples 5 and 6, below. Typically, such cell-permeable Bcl-xL inhibitors will exhibit an EC₅₀ of less than about 500 nM in the Molt-4 cytotoxicity assay of Examples 5 and 6, but may exhibit a significantly lower EC₅₀, for example an EC₅₀ of less than about 250, 100, 50, 20, 10 or even 5 nM.

Owing to the presence of solubilizing groups, many of the Bcl-xL inhibitors described herein are expected to exhibit low or very low cell permeability, and therefore will not yield significant activity in certain cellular assays due to the inability of the compound to traverse the cell membrane, including the Molt-4 cellular toxicity assay of Examples 5 and 6. Bcl-xL inhibitory activity of compounds that do not freely traverse cell membranes may be confirmed in cellular assays with permeabilized cells. The process of mitochondrial outer-membrane permeabilization (MOMP) is controlled by the Bcl-2 family proteins. Specifically, MOMP is promoted by the pro-apoptotic Bcl-2 family proteins Bax and Bak which, upon activation oligomerize on the outer mitochondrial membrane and form pores, leading to release of cytochrome c (cyt c). The release of cyt c triggers formulation of the apoptosome which, in turn, results in caspase activation and other events that commit the cell to undergo programmed cell death (see, Goldstein et al., 2005, Cell Death and Differentiation 12:453-462). The oligomerization action of Bax and Bak is antagonized by the anti-apoptotic Bcl-2 family members, including Bcl-2 and Bcl-xL. Bcl-xL inhibitors, in cells that depend upon Bcl-xL for survival, can cause activation of Bax and/or Bak, MOMP, release of cyt c and downstream events leading to apoptosis. The process of cyt c release can be measured via western blot of both mitochondrial and cytosolic fractions of cells and used as a proxy measurement of apoptosis in cells.

As a means of detecting Bcl-xL inhibitory activity and consequent release of cyt c for Bcl-xL inhibitors with low cell permeability, the cells can be treated with an agent that causes selective pore formation in the plasma, but not mitochondrial, membrane. Specifically, the cholesterol/phospholipid ratio is much higher in the plasma membrane than the mitochondrial membrane. As a result, short incubation with low concentrations of the cholesterol-directed detergent digitonin selectively permeabilizes the plasma membrane without significantly affecting the mitochondrial membrane. This agent forms insoluble complexes with cholesterol leading to the segregation of cholesterol from its normal phospholipid binding sites. This action, in turn, leads to the formation of holes about 40-50 Å wide in the lipid bilayer. Once the plasma membrane is permeabilized, cytosolic components able to pass over digitonin-formed holes can be washed out, including the cytochrome C that was released from mitochondria to cytosol in the apoptotic cells (Campos, 2006, Cytometry A 69(6):515-523).

Typically, Bcl-xL inhibitors will yield an EC₅₀ of less than about 10 nM in the Molt4 cell permeabilized cyt c assay of Examples 5 and 6, although the compounds may exhibit significantly lower EC₅₀s, for example, less than about 5, 1, or even 0.5 nM. As demonstrated in Example 6, Bcl-xL inhibitors having low or very low cell permeability that do not exhibit activity in the standard Molt-4 cellular toxicity assay with non-permeabilized cells exhibit potent functional activity, as measured by release of cyt c, in cellular cytotoxicity assays with permeabilized cells. In addition to cytochrome c release, mitochondria undergoing apoptosis frequently lose their transmembrane mitochondrial membrane potential (Bouchier-Hayes et al., 2008, Methods 44(3): 222-228). JC-1 is a cationic carbocyanine dye that accumulates in mitochondria and fluoresces red when mitochondria are healthy and is lost when the mitochondrial membrane is compromised (percentage depolarization; Smiley et al., 1991, Proc. Natl. Acad. Sci. USA, 88: 3671-3675; Reers et al., 1991: Biochemistry, 30: 4480-4486). This loss in signal can be detected in permeabilized cells using a fluorimeter (excitation 545 nm and emission of 590 nm) and is therefore fully quantitative, enhancing both reproducibility and throughput. Typically, Bcl-xL inhibitors will yield an EC₅₀ of less than about 10 nM in the Molt-4 cell permeabilized JC-1 assay of Examples 5 and 6, although the compounds may exhibit significantly lower EC₅₀s, for example, less than about 5, 1, 0.5 or even 0.05 nM. As demonstrated in Example 6, Bcl-xL inhibitors having low or very low cell permeability that do not exhibit activity in the standard Molt-4 cellular toxicity assay with non-permeabilized cells exhibit potent functional activity, as measured by their loss of transmembrane mitochondrial membrane potential in the JC-1 assay, in cellular cytotoxicity assays with permeabilized cells. Low permeability Bcl-xL inhibitors also exhibit potent activity when administered to cells in the form of ADCs (see, e.g., Example 8).

Although many of the Bcl-xL inhibitors of structural formulae (IIa)-(IId) selectively or specifically inhibit Bcl-xL over other anti-apoptotic Bcl-2 family proteins, selective and/or specific inhibition of Bcl-xL is not necessary. The Bcl-xL inhibitors and ADCs comprising the compounds may also, in addition to inhibiting Bcl-xL, inhibit one or more other anti-apoptotic Bcl-2 family proteins, such as, for example, Bcl-2. In some embodiments, the Bcl-xL inhibitors and/or ADCs are selective and/or specific for Bcl-xL. By specific or selective is meant that the particular Bcl-xL inhibitor and/or ADC binds or inhibits Bcl-xL to a greater extent than Bcl-2 under equivalent assay conditions. In specific embodiments, the Bcl-xL inhibitors and/or ADCs exhibit in the range of about 10-fold, 100-fold, or even greater specificity or selectivity for Bcl-xL than Bcl-2 in binding assays.

### 4.4. Linkers

In the ADCs described herein, the Bcl-xL inhibitors are linked to the antibody by way of linkers. The linker linking a Bcl-xL inhibitor to the antibody of an ADC may be short, long, hydrophobic, hydrophilic, flexible or rigid, or may be composed of segments that each independently have one or more of the above-mentioned properties such that the linker may include segments having different properties. The linkers may be polyvalent such that they covalently link more than one Bcl-xL inhibitor to a single site on the antibody, or monovalent such that covalently they link a single Bcl-xL inhibitor to a single site on the antibody.

As will be appreciated by skilled artisans, the linkers link the Bcl-xL inhibitors to the antibody by forming a covalent linkage to the Bcl-xL inhibitor at one location and a covalent linkage to antibody at another. The covalent linkages are formed by reaction between functional groups on the linker and functional groups on the inhibitors and antibody. As used herein, the expression “linker” is intended to include (i) unconjugated forms of the linker that include a functional group capable of covalently linking the linker to a Bcl-xL inhibitor and a functional group capable of covalently linking the linker to an antibody; (ii) partially conjugated forms of the linker that include a functional group capable of covalently linking the linker to an antibody and that is covalently linked to a Bcl-xL inhibitor, or vice versa; and (iii) fully conjugated forms of the linker that is covalently linked to both a Bcl-xL inhibitor and an antibody. In some specific embodiments of intermediate synthons and ADCs described herein, moieties comprising the functional groups on the linker and covalent linkages formed between the linker and antibody are specifically illustrated as R^{x} and LK, respectively.

The linkers are preferably, but need not be, chemically stable to conditions outside the cell, and may be designed to cleave, immolate and/or otherwise specifically degrade inside the cell. Alternatively, linkers that are not designed to specifically cleave or degrade inside the cell may be used. A wide variety of linkers useful for linking drugs to antibodies in the context of ADCs are known in the art. Any of these linkers, as well as other linkers, may be used to link the Bcl-xL inhibitors to the antibody of the ADCs described herein.

Exemplary polyvalent linkers that may be used to link many Bcl-xL inhibitors to an antibody are described, for example, in U.S. Pat. No. 8,399,512; U.S. Published Application No. 2010/0152725; U.S. Pat. Nos. 8,524,214; 8,349,308; U.S. Published Application No. 2013/189218; U.S. Published Application No. 2014/017265; WO 2014/093379; WO 2014/093394; WO 2014/093640, the contents of which are incorporated herein by reference in their entireties. For example, the Fleximer® linker technology developed by Mersana et al. has the potential to enable high-DAR ADCs with good physicochemical properties. As shown below, the Fleximer® linker technology is based on incorporating drug molecules into a solubilizing poly-acetal backbone via a sequence of ester bonds. The methodology renders highly-loaded ADCs (DAR up to 20) whilst maintaining good physicochemical properties. This methodology could be utilized with Bcl-xL inhibitors as shown in the Scheme below.

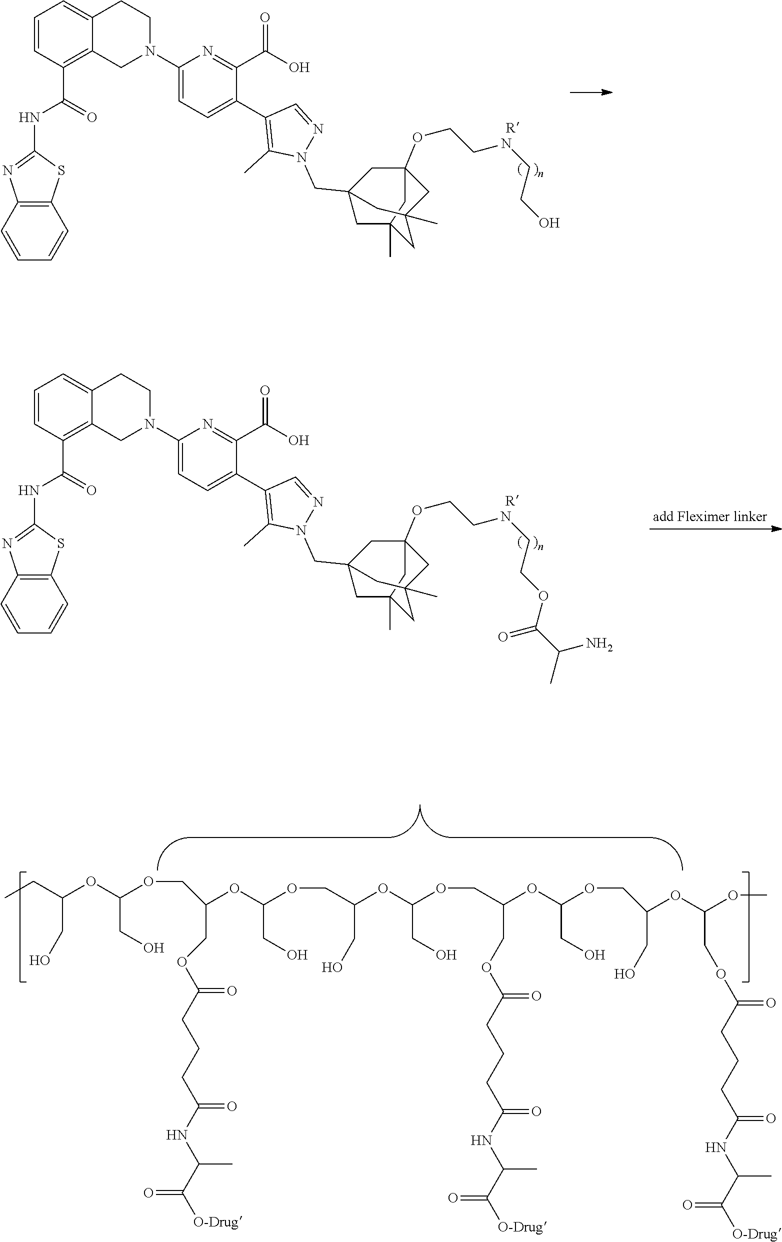

To utilize the Fleximer® linker technology depicted in the scheme above, an aliphatic alcohol can be present or introduced into the Bcl-xL inhibitor. The alcohol moiety is then conjugated to an alanine moiety, which is then synthetically incorporated into the Fleximer® linker. Liposomal processing of the ADC in vitro releases the parent alcohol-containing drug.

Additional examples of dendritic type linkers can be found in US 2006/116422; US 2005/271615; de Groot et al., (2003) Angew. Chem. Int. Ed. 42:4490-4494; Amir et al., (2003) Angew. Chem. Int. Ed. 42:4494-4499; Shamis et al., (2004) J. Am. Chem. Soc. 126:1726-1731; Sun et al., (2002) Bioorganic & Medicinal Chemistry Letters 12:2213-2215; Sun et al., (2003) Bioorganic & Medicinal Chemistry 11:1761-1768; King et al., (2002) Tetrahedron Letters 43:1987-1990.

Exemplary monovalent linkers that may be used are described, for example, in Nolting, 2013, Antibody-Drug Conjugates, Methods in Molecular Biology 1045:71-100; Kitson et al., 2013, CROs/CMOs—Chemica Oggi—Chemistry Today 31(4): 30-36; Ducry et al., 2010, Bioconjugate Chem. 21:5-13; Zhao et al., 2011, J. Med. Chem. 54:3606-3623; U.S. Pat. Nos. 7,223,837; 8,568,728; 8,535,678; and WO2004010957, the content of each of which is incorporated herein by reference in their entireties.

By way of example and not limitation, some cleavable and noncleavable linkers that may be included in the ADCs described herein are described below.

### 4.4.1.1. Cleavable Linkers

In certain embodiments, the linker selected is cleavable in vitro and in vivo. Cleavable linkers may include chemically or enzymatically unstable or degradable linkages. Cleavable linkers generally rely on processes inside the cell to liberate the drug, such as reduction in the cytoplasm, exposure to acidic conditions in the lysosome, or cleavage by specific proteases or other enzymes within the cell. Cleavable linkers generally incorporate one or more chemical bonds that are either chemically or enzymatically cleavable while the remainder of the linker is noncleavable.

In certain embodiments, a linker comprises a chemically labile group such as hydrazone and/or disulfide groups. Linkers comprising chemically labile groups exploit differential properties between the plasma and some cytoplasmic compartments. The intracellular conditions to facilitate drug release for hydrazone containing linkers are the acidic environment of endosomes and lysosomes, while the disulfide containing linkers are reduced in the cytosol, which contains high thiol concentrations, e.g., glutathione. In certain embodiments, the plasma stability of a linker comprising a chemically labile group may be increased by introducing steric hindrance using substituents near the chemically labile group.

Acid-labile groups, such as hydrazone, remain intact during systemic circulation in the blood's neutral pH environment (pH 7.3-7.5) and undergo hydrolysis and release the drug once the ADC is internalized into mildly acidic endosomal (pH 5.0-6.5) and lysosomal (pH 4.5-5.0) compartments of the cell. This pH dependent release mechanism has been associated with nonspecific release of the drug. To increase the stability of the hydrazone group of the linker, the linker may be varied by chemical modification, e.g., substitution, allowing tuning to achieve more efficient release in the lysosome with a minimized loss in circulation.

Hydrazone-containing linkers may contain additional cleavage sites, such as additional acid-labile cleavage sites and/or enzymatically labile cleavage sites. ADCs including exemplary hydrazone-containing linkers include the following structures:

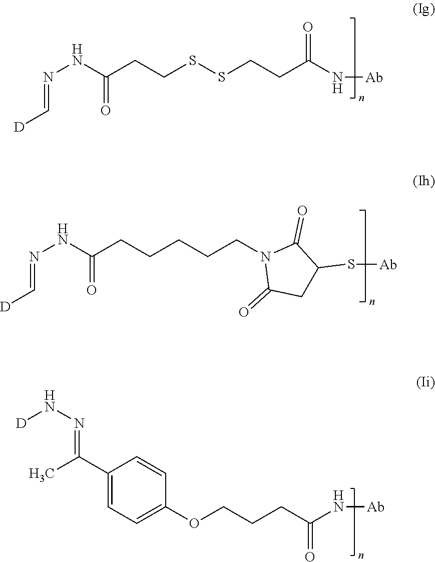

wherein D and Ab represent the drug and Ab, respectively, and n represents the number of drug-linkers linked to the antibody. In certain linkers such as linker (Ig), the linker comprises two cleavable groups—a disulfide and a hydrazone moiety. For such linkers, effective release of the unmodified free drug requires acidic pH or disulfide reduction and acidic pH. Linkers such as (Ih) and (Ii) have been shown to be effective with a single hydrazone cleavage site.

Other acid-labile groups that may be included in linkers include cis-aconityl-containing linkers. cis-Aconityl chemistry uses a carboxylic acid juxtaposed to an amide bond to accelerate amide hydrolysis under acidic conditions.

Cleavable linkers may also include a disulfide group. Disulfides are thermodynamically stable at physiological pH and are designed to release the drug upon internalization inside cells, wherein the cytosol provides a significantly more reducing environment compared to the extracellular environment. Scission of disulfide bonds generally requires the presence of a cytoplasmic thiol cofactor, such as (reduced) glutathione (GSH), such that disulfide-containing linkers are reasonable stable in circulation, selectively releasing the drug in the cytosol. The intracellular enzyme protein disulfide isomerase, or similar enzymes capable of cleaving disulfide bonds, may also contribute to the preferential cleavage of disulfide bonds inside cells. GSH is reported to be present in cells in the concentration range of 0.5-10 mM compared with a significantly lower concentration of GSH or cysteine, the most abundant low-molecular weight thiol, in circulation at approximately 5 μM. Tumor cells, where irregular blood flow leads to a hypoxic state, result in enhanced activity of reductive enzymes and therefore even higher glutathione concentrations. In certain embodiments, the in vivo stability of a disulfide-containing linker may be enhanced by chemical modification of the linker, e.g., use of steric hindrance adjacent to the disulfide bond.

ADCs including exemplary disulfide-containing linkers include the following structures:

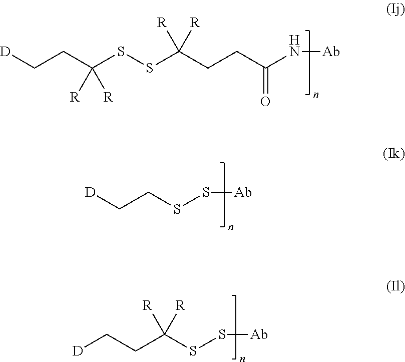

wherein D and Ab represent the drug and antibody, respectively, n represents the number of drug-linkers linked to the antibody and R is independently selected at each occurrence from hydrogen or alkyl, for example. In certain embodiments, increasing steric hindrance adjacent to the disulfide bond increases the stability of the linker. Structures such as (Ij) and (Il) show increased in vivo stability when one or more R groups is selected from a lower alkyl such as methyl.

Another type of linker that may be used is a linker that is specifically cleaved by an enzyme. Such linkers are typically peptide-based or include peptidic regions that act as substrates for enzymes. Peptide based linkers tend to be more stable in plasma and extracellular millieu than chemically labile linkers. Peptide bonds generally have good serum stability, as lysosomal proteolytic enzymes have very low activity in blood due to endogenous inhibitors and the unfavorably high pH value of blood compared to lysosomes. Release of a drug from an antibody occurs specifically due to the action of lysosomal proteases, e.g., cathepsin and plasmin. These proteases may be present at elevated levels in certain tumor tissues. In certain embodiments, the linker is cleavable by a lysosomal enzyme. In certain embodiments, the linker is cleavable by a lysosomal enzyme, and the lysosomal enzyme is Cathepsin B. In certain embodiments, the linker is cleavable by a lysosomal enzyme, and the lysosomal enzyme is β-glucuronidase or β-galactosidase. In certain embodiments, the linker is cleavable by a lysosomal enzyme, and the lysosomal enzyme is β-glucuronidase. In certain embodiments, the linker is cleavable by a lysosomal enzyme, and the lysosomal enzyme is β-galactosidase.

Those skilled in the art recognize the importance of cleavable linkers that are stable to plasma, yet are readily cleaved by a lysosomal enzyme. Disclosed herein, in certain embodiments, are linkers, cleavable by the lysosomal enzymes β-glucuronidase or β-galactosidase, that show improved plasma stability and reduced non-specific release of small molecule drug.

In exemplary embodiments, the cleavable peptide is selected from tetrapeptides such as Gly-Phe-Leu-Gly, Ala-Leu-Ala-Leu or dipeptides such as Val-Cit, Val-Ala, and Phe-Lys. In certain embodiments, dipeptides are preferred over longer polypeptides due to hydrophobicity of the longer peptides.

A variety of dipeptide-based cleavable linkers useful for linking drugs such as doxorubicin, mitomycin, camptothecin, tallysomycin and auristatin/auristatin family members to antibodies have been described (see, Dubowchik et al., 1998, J. Org. Chem. 67:1866-1872; Dubowchik et al., 1998, Bioorg. Med. Chem. Lett. 8:3341-3346; Walker et al., 2002, Bioorg. Med. Chem. Lett. 12:217-219; Walker et al., 2004, Bioorg. Med. Chem. Lett. 14:4323-4327; and Francisco et al., 2003, Blood 102:1458-1465, the contents of each of which are incorporated herein by reference). All of these dipeptide linkers, or modified versions of these dipeptide linkers, may be used in the ADCs described herein. Other dipeptide linkers that may be used include those found in ADCs such as Seattle Genetics' Brentuximab Vendotin SGN-35 (Adcetris™), Seattle Genetics SGN-75 (anti-CD-70, MC-monomethyl auristatin F (MMAF), Celldex Therapeutics glembatumumab (CDX-011) (anti-NMB, Val-Cit-monomethyl auristatin E (MMAE), and Cytogen PSMA-ADC (PSMA-ADC-1301) (anti-PSMA, Val-Cit-MMAE).

Enzymatically cleavable linkers may include a self-immolative spacer to spatially separate the drug from the site of enzymatic cleavage. The direct attachment of a drug to a peptide linker can result in proteolytic release of an amino acid adduct of the drug, thereby impairing its activity. The use of a self-immolative spacer allows for the elimination of the fully active, chemically unmodified drug upon amide bond hydrolysis.

One self-immolative spacer is the bifunctional para-aminobenzyl alcohol group, which is linked to the peptide through the amino group, forming an amide bond, while amine containing drugs may be attached through carbamate functionalities to the benzylic hydroxyl group of the linker (to give a p-amidobenzylcarbamate, PABC). The resulting prodrugs are activated upon protease-mediated cleavage, leading to a 1,6-elimination reaction releasing the unmodified drug, carbon dioxide, and remnants of the linker group. The following scheme depicts the fragmentation of p-amidobenzyl carbamate and release of the drug:

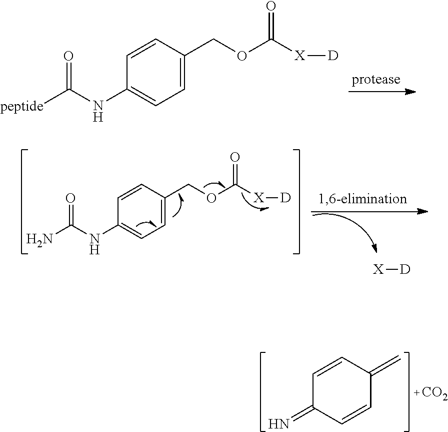

wherein X-D represents the unmodified drug.

Heterocyclic variants of this self-immolative group have also been described. See U.S. Pat. No. 7,989,434.

In certain embodiments, the enzymatically cleavable linker is a ß-glucuronic acid-based linker. Facile release of the drug may be realized through cleavage of the ß-glucuronide glycosidic bond by the lysosomal enzyme ß-glucuronidase. This enzyme is present abundantly within lysosomes and is overexpressed in some tumor types, while the enzyme activity outside cells is low. ß-Glucuronic acid-based linkers may be used to circumvent the tendency of an ADC to undergo aggregation due to the hydrophilic nature of ß-glucuronides. In certain embodiments, ß-glucuronic acid-based linkers are preferred as linkers for ADCs linked to hydrophobic drugs. The following scheme depicts the release of the drug from and ADC containing a ß-glucuronic acid-based linker:

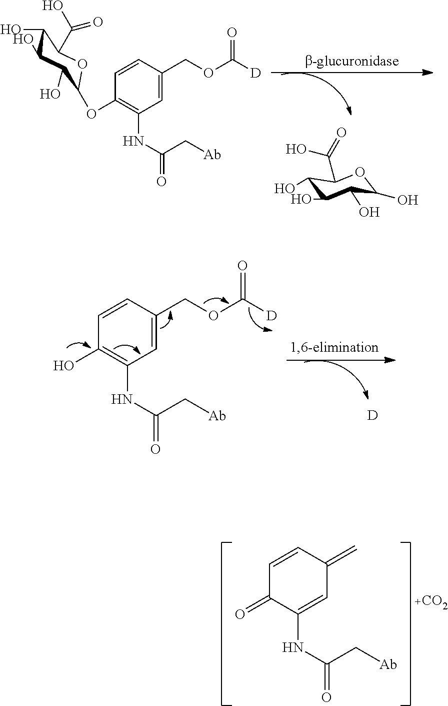

A variety of cleavable ß-glucuronic acid-based linkers useful for linking drugs such as auristatins, camptothecin and doxorubicin analogues, CBI minor-groove binders, and psymberin to antibodies have been described (see, Jeffrey et al., 2006, Bioconjug. Chem. 17:831-840; Jeffrey et al., Bioorg. Med. Chem. Lett. 17:2278-2280; and Jiang et al., 2005, J. Am. Chem. Soc. 127:11254-11255, the contents of each of which are incorporated herein by reference). All of these ß-glucuronic acid-based linkers may be used in the ADCs described herein. In certain embodiments, the enzymatically cleavable linker is a ß-galactoside-based linker. ß-Galactoside is present abundantly within lysosomes, while the enzyme activity outside cells is low. Additionally, Bcl-xL inhibitors containing a phenol group can be covalently bonded to a linker through the phenolic oxygen. One such linker, described in U.S. Published App. No. 2009/0318668, relies on a methodology in which a diamino-ethane “SpaceLink” is used in conjunction with traditional “PABO”-based self-immolative groups to deliver phenols. The cleavage of the linker is depicted schematically below using a Bcl-xL inhibitor of the disclosure.

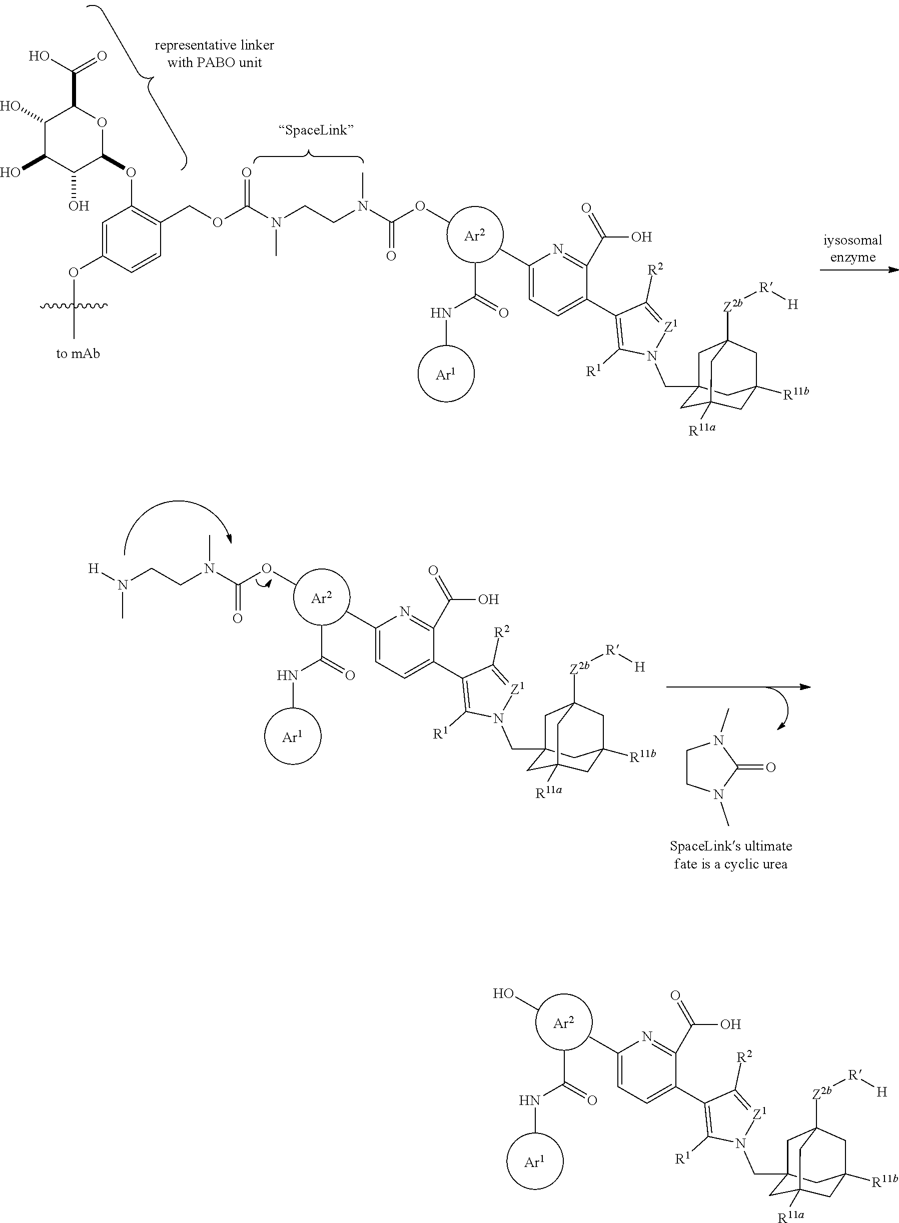

Cleavable linkers may include noncleavable portions or segments, and/or cleavable segments or portions may be included in an otherwise non-cleavable linker to render it cleavable. By way of example only, polyethylene glycol (PEG) and related polymers may include cleavable groups in the polymer backbone. For example, a polyethylene glycol or polymer linker may include one or more cleavable groups such as a disulfide, a hydrazone or a dipeptide.

Other degradable linkages that may be included in linkers include ester linkages formed by the reaction of PEG carboxylic acids or activated PEG carboxylic acids with alcohol groups on a biologically active agent, wherein such ester groups generally hydrolyze under physiological conditions to release the biologically active agent. Hydrolytically degradable linkages include, but are not limited to, carbonate linkages; imine linkages resulting from reaction of an amine and an aldehyde; phosphate ester linkages formed by reacting an alcohol with a phosphate group; acetal linkages that are the reaction product of an aldehyde and an alcohol; orthoester linkages that are the reaction product of a formate and an alcohol; and oligonucleotide linkages formed by a phosphoramidite group, including but not limited to, at the end of a polymer, and a 5′ hydroxyl group of an oligonucleotide.

In certain embodiments, the linker comprises an enzymatically cleavable peptide moiety, for example, a linker comprising structural formula (IVa), (IVb), (IVc) or (IVd):

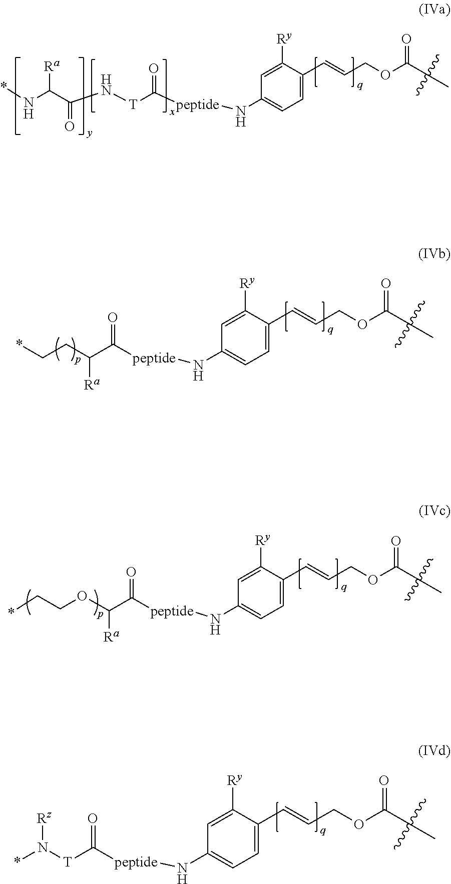

or a salt thereof, wherein:
peptide represents a peptide (illustrated N→C, wherein peptide includes the amino and carboxy “termini”) cleavable by a lysosomal enzyme;
T represents a polymer comprising one or more ethylene glycol units or an alkylene chain, or combinations thereof;
R^{a} is selected from hydrogen, alkyl, sulfonate and methyl sulfonate;
R^{y} is hydrogen or C₁₋₄ alkyl-(O)ᵣ(C₁₋₄ alkylene)ₛ-G¹ or C₁₋₄ alkyl-(N)—[(C₁₋₄ alkylene)-G¹]₂;
R^{z} is C₁₋₄ alkyl-(O)ᵣ—(C₁₋₄ alkylene)ₛ-G²;
G¹ is SO₃H, CO₂H, PEG 4-32, or sugar moiety;
G² is SO₃H, CO₂H, or PEG 4-32 moiety;
r is 0 or 1;
s is 0 or 1;
p is an integer ranging from 0 to 5;
q is 0 or 1;
x is 0 or 1;
y is 0 or 1;
[symbol] represents the point of attachment of the linker to the Bcl-xL inhibitor; and
* represents the point of attachment to the remainder of the linker.

In certain embodiments, the linker comprises an enzymatically cleavable peptide moiety, for example, a linker comprising structural formula (IVa), (IVb), (IVc), or (IVd), or salts thereof.

In certain embodiments, the peptide is selected from a tripeptide or a dipeptide. In particular embodiments, the dipeptide is selected from: Val-Cit; Cit-Val; Ala-Ala; Ala-Cit; Cit-Ala; Asn-Cit; Cit-Asn; Cit-Cit; Val-Glu; Glu-Val; Ser-Cit; Cit-Ser; Lys-Cit; Cit-Lys; Asp-Cit; Cit-Asp; Ala-Val; Val-Ala; Phe-Lys; Lys-Phe; Val-Lys; Lys-Val; Ala-Lys; Lys-Ala; Phe-Cit; Cit-Phe; Leu-Cit; Cit-Leu; Ile-Cit; Cit-Ile; Phe-Arg; Arg-Phe; Cit-Trp; and Trp-Cit; or salts thereof.

Exemplary embodiments of linkers according to structural formula (IVa) that may be included in the ADCs described herein include the linkers illustrated below (as illustrated, the linkers include a group suitable for covalently linking the linker to an antibody):

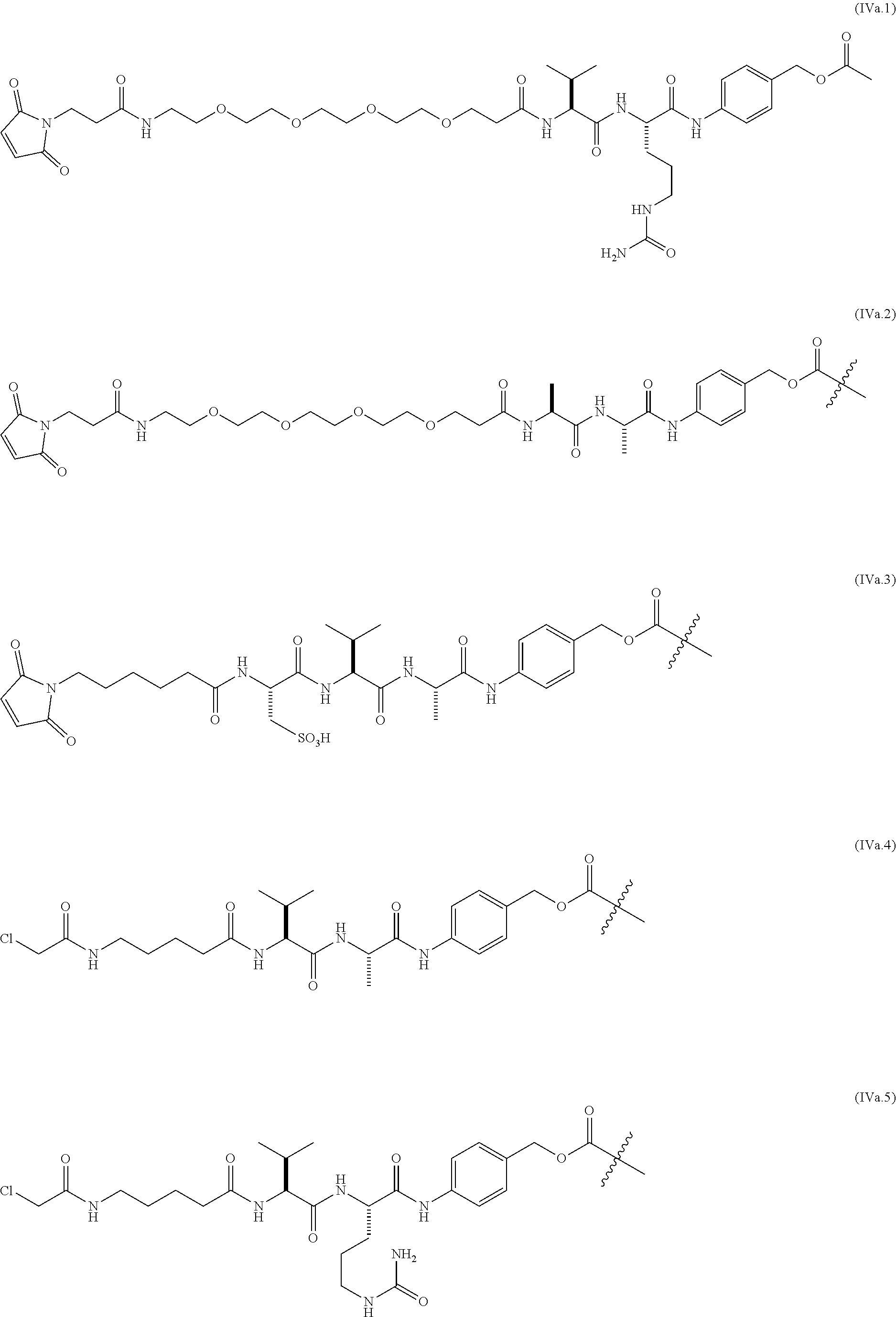 [SMILES: CC(=O)OCc1ccc(NC(=O)[C@H](CCCNC(N)=O)NC(=O)[C@@H](NC(=O)CCOCCOCCOCCOCCNC(=O)CCN2C(=O)C=CC2=O)C(C)C)cc1 ; *C(=O)OCc1ccc(NC(=O)[C@H](C)NC(=O)[C@H](C)NC(=O)CCOCCOCCOCCOCCNC(=O)CCN2C(=O)C=CC2=O)cc1 ; *C(=O)OCc1ccc(NC(=O)[C@H](C)NC(=O)[C@@H](NC(=O)[C@H](CS(=O)(=O)O)NC(=O)CCCCCN2C(=O)C=CC2=O)C(C)C)cc1 ; *C(=O)OCc1ccc(NC(=O)[C@H](C)NC(=O)[C@@H](NC(=O)CCCCNC(=O)CCl)C(C)C)cc1 ; *C(=O)OCc1ccc(NC(=O)[C@H](CCCNC(N)=O)NC(=O)[C@@H](NC(=O)CCCCNC(=O)CCl)C(C)C)cc1]

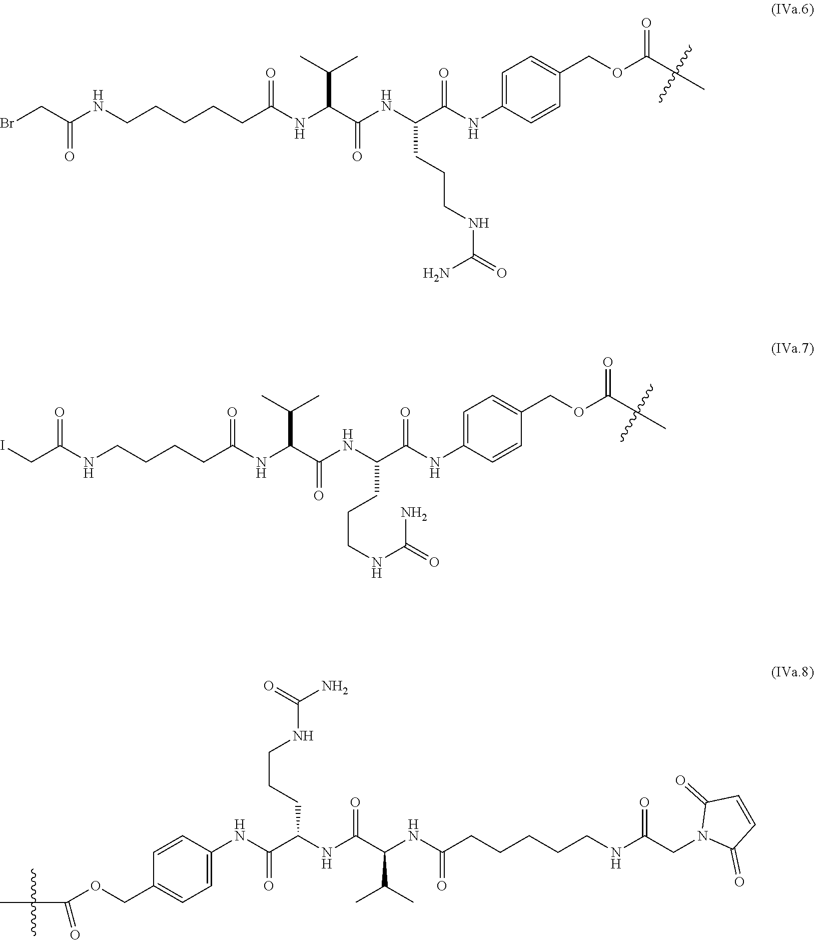 [SMILES: *C(=O)OCc1ccc(NC(=O)[C@H](CCCNC(N)=O)NC(=O)[C@@H](NC(=O)CCCCCNC(=O)CBr)C(C)C)cc1 ; *C(=O)OCc1ccc(NC(=O)[C@H](CCCNC(N)=O)NC(=O)[C@@H](NC(=O)CCCCNC(=O)CI)C(C)C)cc1 ; *C(=O)OCc1ccc(NC(=O)[C@H](CCCNC(N)=O)NC(=O)[C@@H](NC(=O)CCCCCNC(=O)CN2C(=O)C=CC2=O)C(C)C)cc1]

Exemplary embodiments of linkers according to structural formula (IVb), (IVc), or (IVd) that may be included in the ADCs described herein include the linkers illustrated below (as illustrated, the linkers include a group suitable for covalently linking the linker to an antibody):

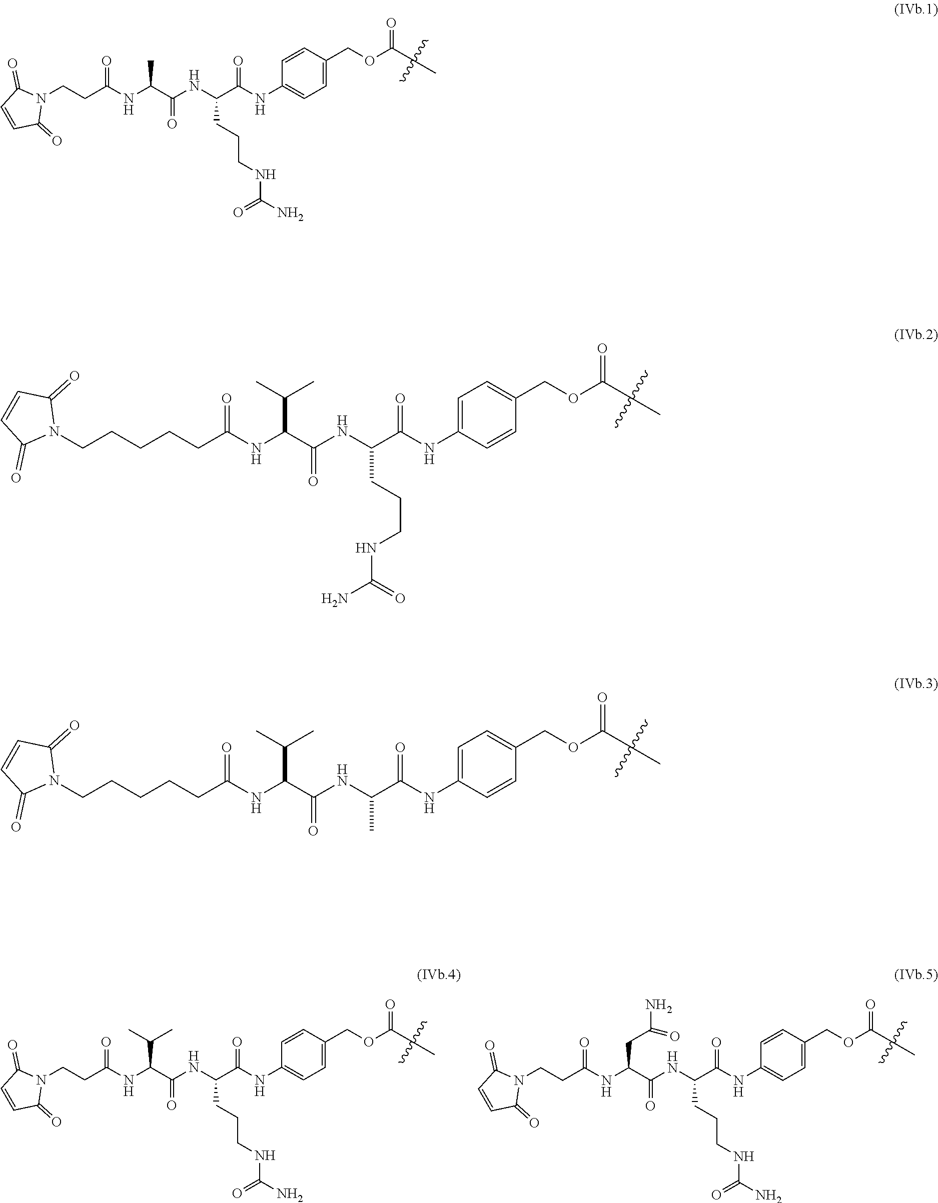 [SMILES: *C(=O)OCc1ccc(NC(=O)[C@H](CCCNC(N)=O)NC(=O)[C@H](C)NC(=O)CCN2C(=O)C=CC2=O)cc1 ; *C(=O)OCc1ccc(NC(=O)[C@H](CCCNC(N)=O)NC(=O)[C@@H](NC(=O)CCCCCN2C(=O)C=CC2=O)C(C)C)cc1 ; *C(=O)OCc1ccc(NC(=O)[C@H](C)NC(=O)[C@@H](NC(=O)CCCCCN2C(=O)C=CC2=O)C(C)C)cc1 ; *C(=O)OCc1ccc(NC(=O)[C@H](CCCNC(N)=O)NC(=O)[C@@H](NC(=O)CCN2C(=O)C=CC2=O)C(C)C)cc1 ; *C(=O)OCc1ccc(NC(=O)[C@H](CCCNC(N)=O)NC(=O)[C@H](CC(N)=O)NC(=O)CCN2C(=O)C=CC2=O)cc1]

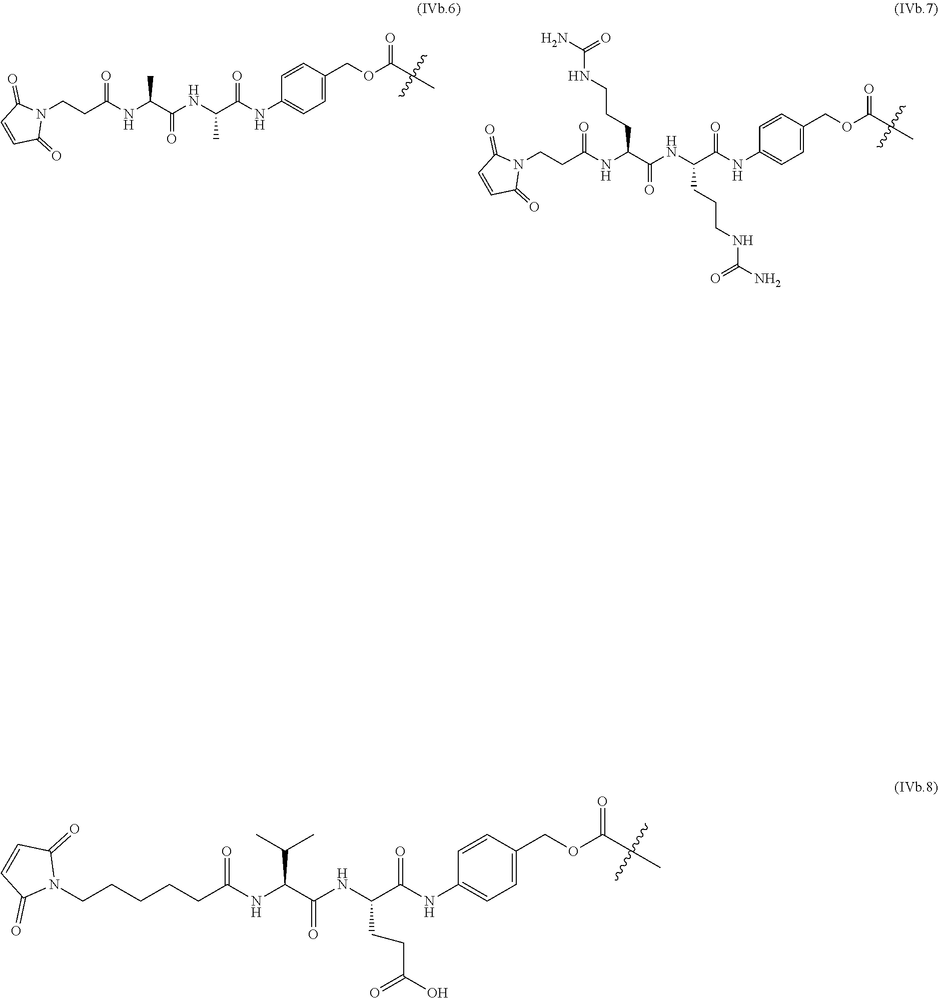 [SMILES: *C(=O)OCc1ccc(NC(=O)[C@H](C)NC(=O)[C@H](C)NC(=O)CCN2C(=O)C=CC2=O)cc1 ; *C(=O)OCc1ccc(NC(=O)[C@H](CCCNC(N)=O)NC(=O)[C@H](CCCNC(N)=O)NC(=O)CCN2C(=O)C=CC2=O)cc1 ; *C(=O)OCc1ccc(NC(=O)[C@H](CCC(=O)O)NC(=O)[C@@H](NC(=O)CCCCCN2C(=O)C=CC2=O)C(C)C)cc1]

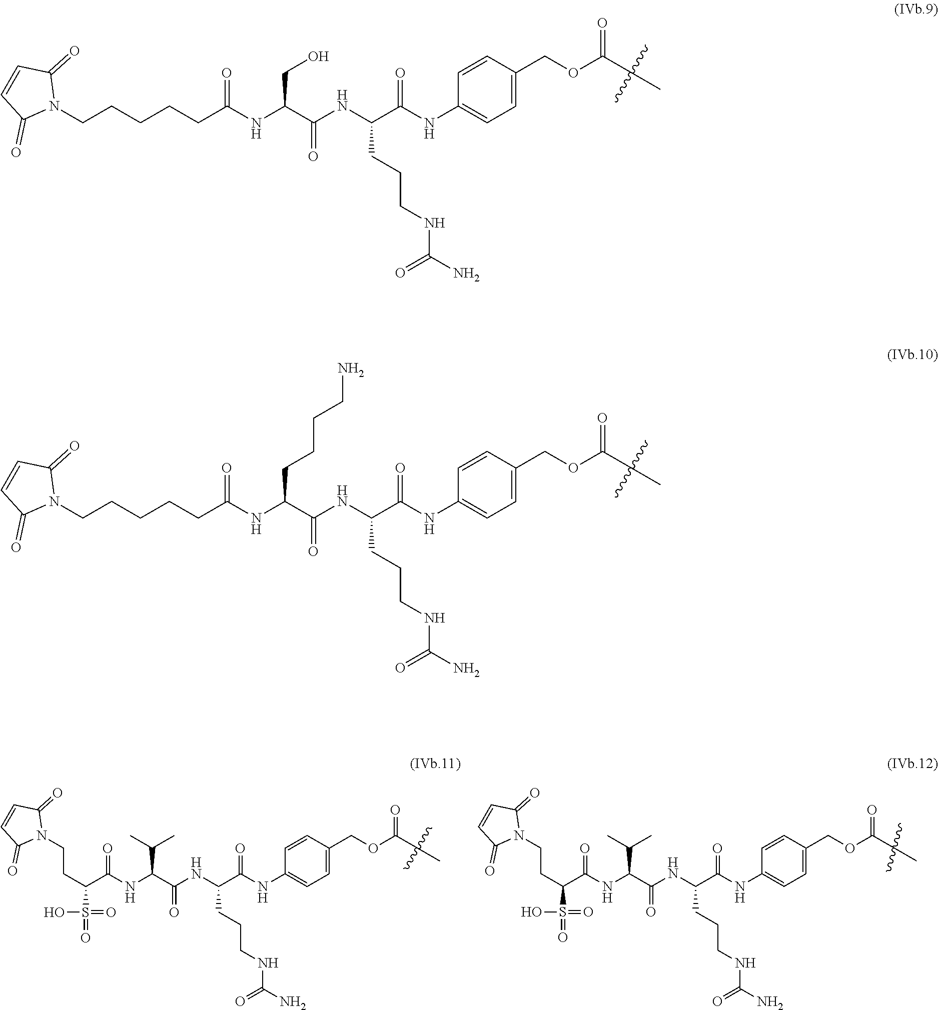 [SMILES: *C(=O)OCc1ccc(NC(=O)[C@H](CCCNC(N)=O)NC(=O)[C@H](CO)NC(=O)CCCCCN2C(=O)C=CC2=O)cc1 ; *C(=O)OCc1ccc(NC(=O)[C@H](CCCNC(N)=O)NC(=O)[C@H](CCCCN)NC(=O)CCCCCN2C(=O)C=CC2=O)cc1 ; *C(=O)OCc1ccc(NC(=O)[C@H](CCCNC(N)=O)NC(=O)[C@@H](NC(=O)[C@@H](CCN2C(=O)C=CC2=O)S(=O)(=O)O)C(C)C)cc1 ; *C(=O)OCc1ccc(NC(=O)[C@H](CCCNC(N)=O)NC(=O)[C@@H](NC(=O)[C@H](CCN2C(=O)C=CC2=O)S(=O)(=O)O)C(C)C)cc1]

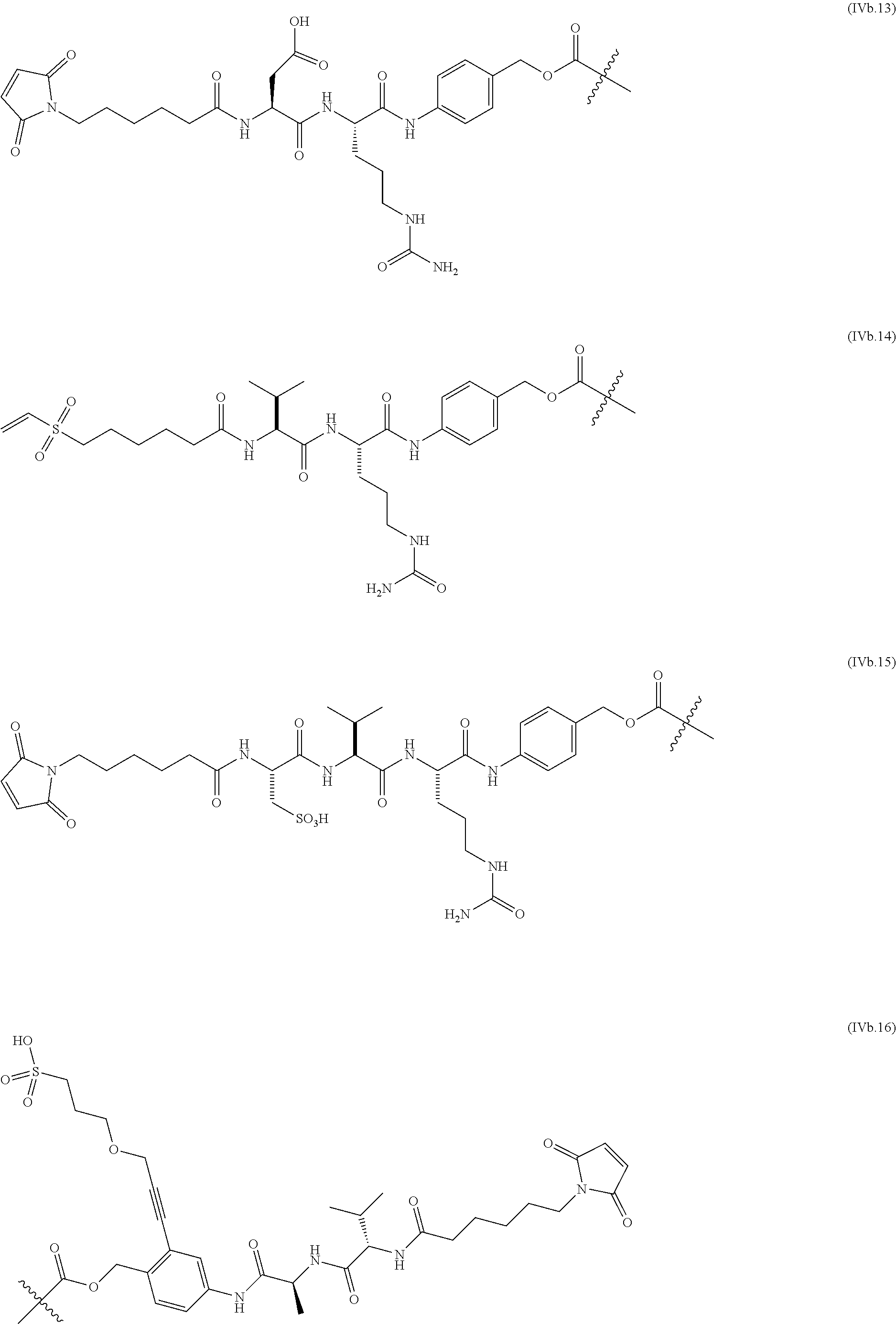 [SMILES: *C(=O)OCc1ccc(NC(=O)[C@H](CCCNC(N)=O)NC(=O)[C@H](CC(=O)O)NC(=O)CCCCCN2C(=O)C=CC2=O)cc1 ; *C(=O)OCc1ccc(NC(=O)[C@H](CCCNC(N)=O)NC(=O)[C@@H](NC(=O)CCCCCS(=O)(=O)C=C)C(C)C)cc1 ; *C(=O)OCc1ccc(NC(=O)[C@H](CCCNC(N)=O)NC(=O)[C@@H](NC(=O)[C@H](CS(=O)(=O)O)NC(=O)CCCCCN2C(=O)C=CC2=O)C(C)C)cc1 ; *C(=O)OCc1ccc(NC(=O)[C@H](C)NC(=O)[C@@H](NC(=O)CCCCCN2C(=O)C=CC2=O)C(C)C)cc1C#CCOCCCS(=O)(=O)O]

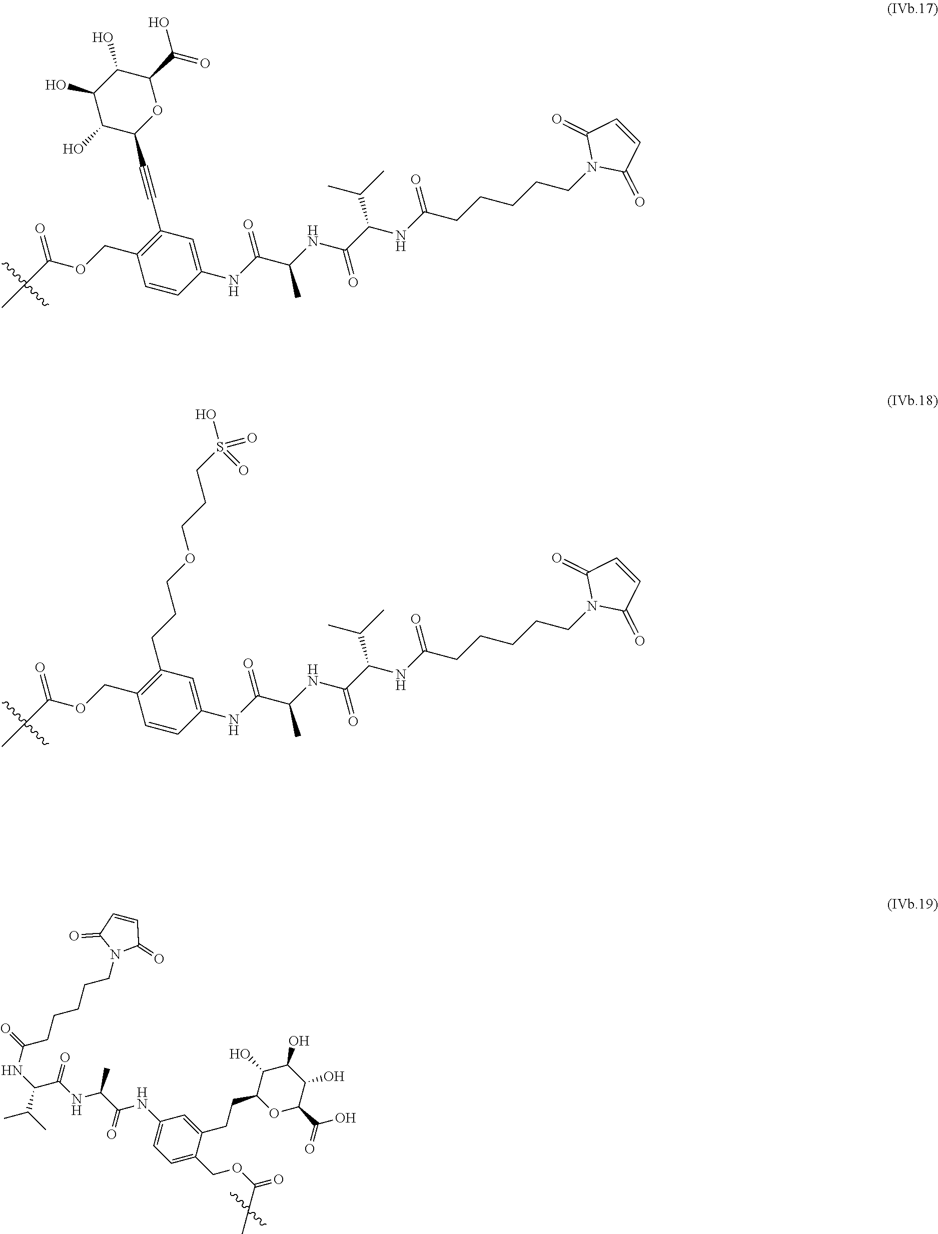 [SMILES: *C(=O)OCc1ccc(NC(=O)[C@H](C)NC(=O)[C@@H](NC(=O)CCCCCN2C(=O)C=CC2=O)C(C)C)cc1C#C[C@@H]1O[C@H](C(=O)O)[C@@H](O)[C@H](O)[C@H]1O ; *C(=O)OCc1ccc(NC(=O)[C@H](C)NC(=O)[C@@H](NC(=O)CCCCCN2C(=O)C=CC2=O)C(C)C)cc1CCCOCCCS(=O)(=O)O ; *C(=O)OCc1ccc(NC(=O)[C@H](C)NC(=O)[C@@H](NC(=O)CCCCCN2C(=O)C=CC2=O)C(C)C)cc1CC[C@@H]1O[C@H](C(=O)O)[C@@H](O)[C@H](O)[C@H]1O]

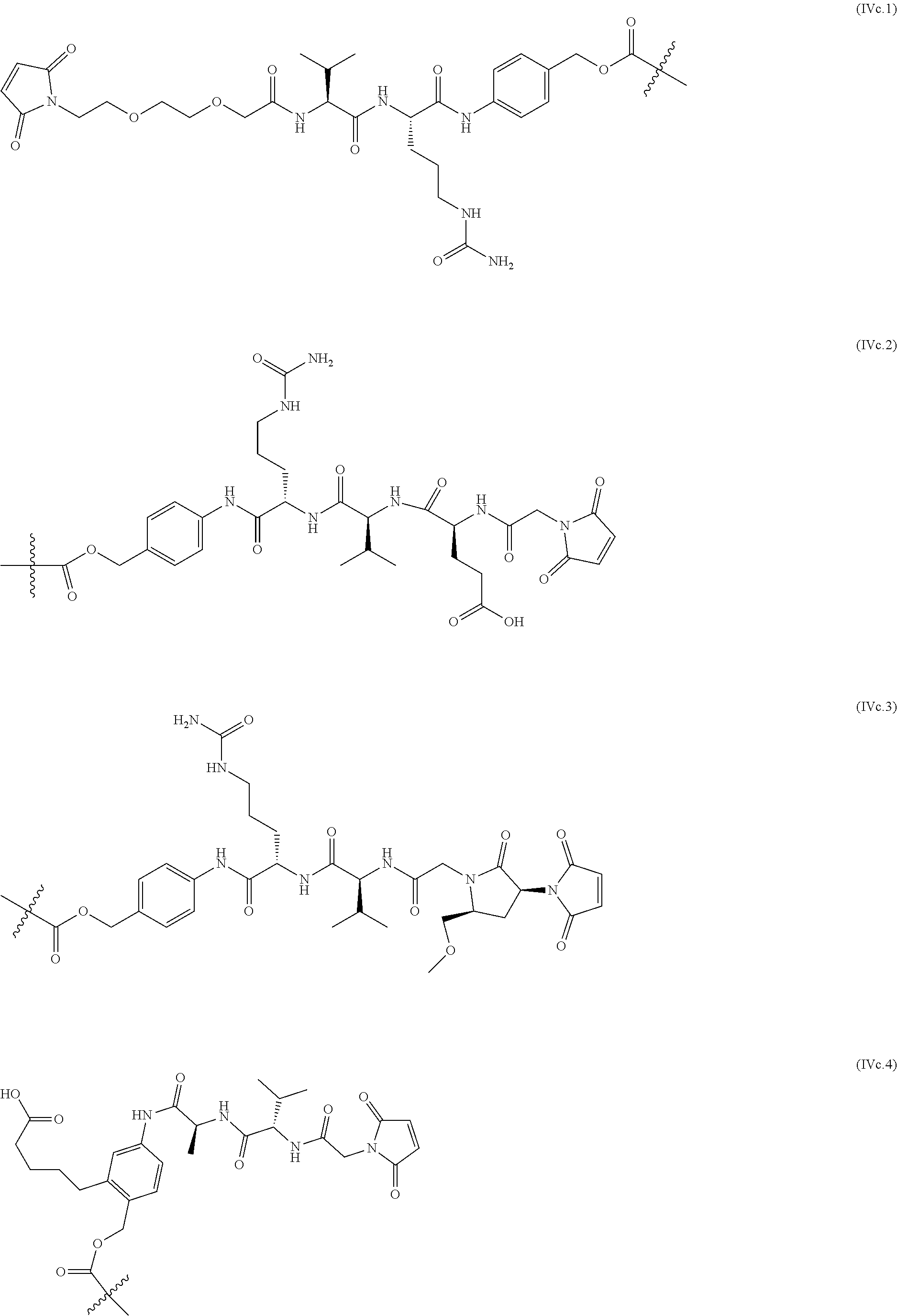 [SMILES: *C(=O)OCc1ccc(NC(=O)[C@H](CCCNC(N)=O)NC(=O)[C@@H](NC(=O)COCCOCCN2C(=O)C=CC2=O)C(C)C)cc1 ; *C(=O)OCc1ccc(NC(=O)[C@H](CCCNC(N)=O)NC(=O)[C@@H](NC(=O)[C@H](CCC(=O)O)NC(=O)CN2C(=O)C=CC2=O)C(C)C)cc1 ; *C(=O)OCc1ccc(NC(=O)[C@H](CCCNC(N)=O)NC(=O)[C@@H](NC(=O)CN2C(=O)[C@@H](N3C(=O)C=CC3=O)C[C@H]2COC)C(C)C)cc1 ; *C(=O)OCc1ccc(NC(=O)[C@H](C)NC(=O)[C@@H](NC(=O)CN2C(=O)C=CC2=O)C(C)C)cc1CCCCC(=O)O]

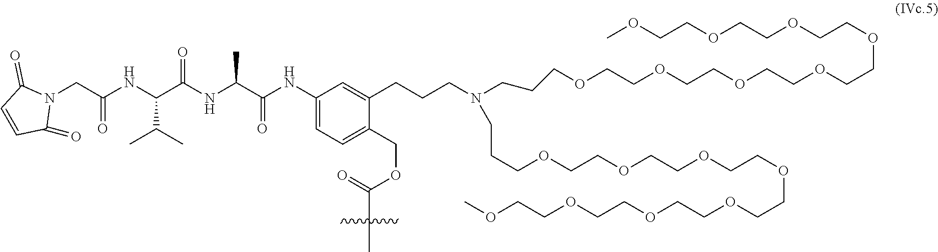 [SMILES: *C(=O)OCc1ccc(NC(=O)[C@H](C)NC(=O)[C@@H](NC(=O)CN2C(=O)C=CC2=O)C(C)C)cc1CCCN(CCCOCCOCCOCCOCCOCCOCCOCCOC)CCCOCCOCCOCCOCCOCCOCCOCCOC]

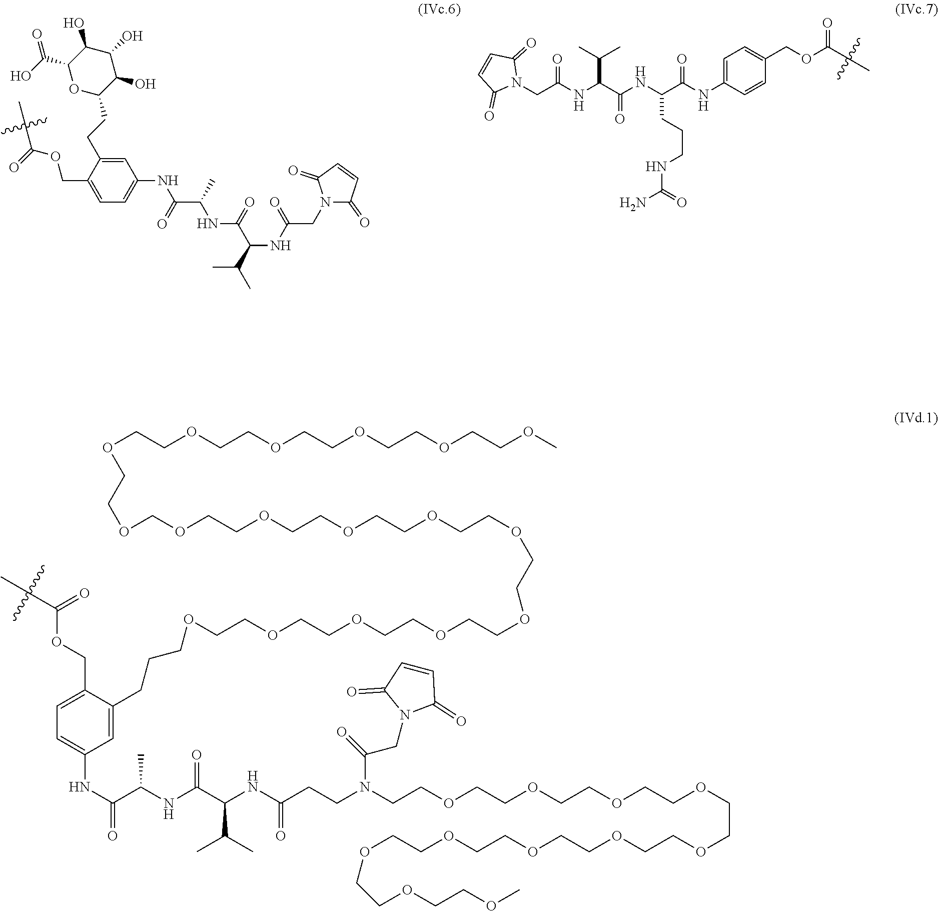 [SMILES: *C(=O)OCc1ccc(NC(=O)[C@H](C)NC(=O)[C@@H](NC(=O)CN2C(=O)C=CC2=O)C(C)C)cc1CC[C@@H]1O[C@H](C(=O)O)[C@@H](O)[C@H](O)[C@H]1O ; *C(=O)OCc1ccc(NC(=O)[C@H](CCCNC(N)=O)NC(=O)[C@@H](NC(=O)CN2C(=O)C=CC2=O)C(C)C)cc1 ; *C(=O)OCc1ccc(NC(=O)[C@H](C)NC(=O)[C@@H](NC(=O)CCN(CCOCCOCCOCCOCCOCCOCCOCCOCCOCCOCCOC)C(=O)CN2C(=O)C=CC2=O)C(C)C)cc1CCCOCCOCCOCCOCCOCCOCCOCCOCCOCCOCOCCOCCOCCOCCOCCOCCOC]

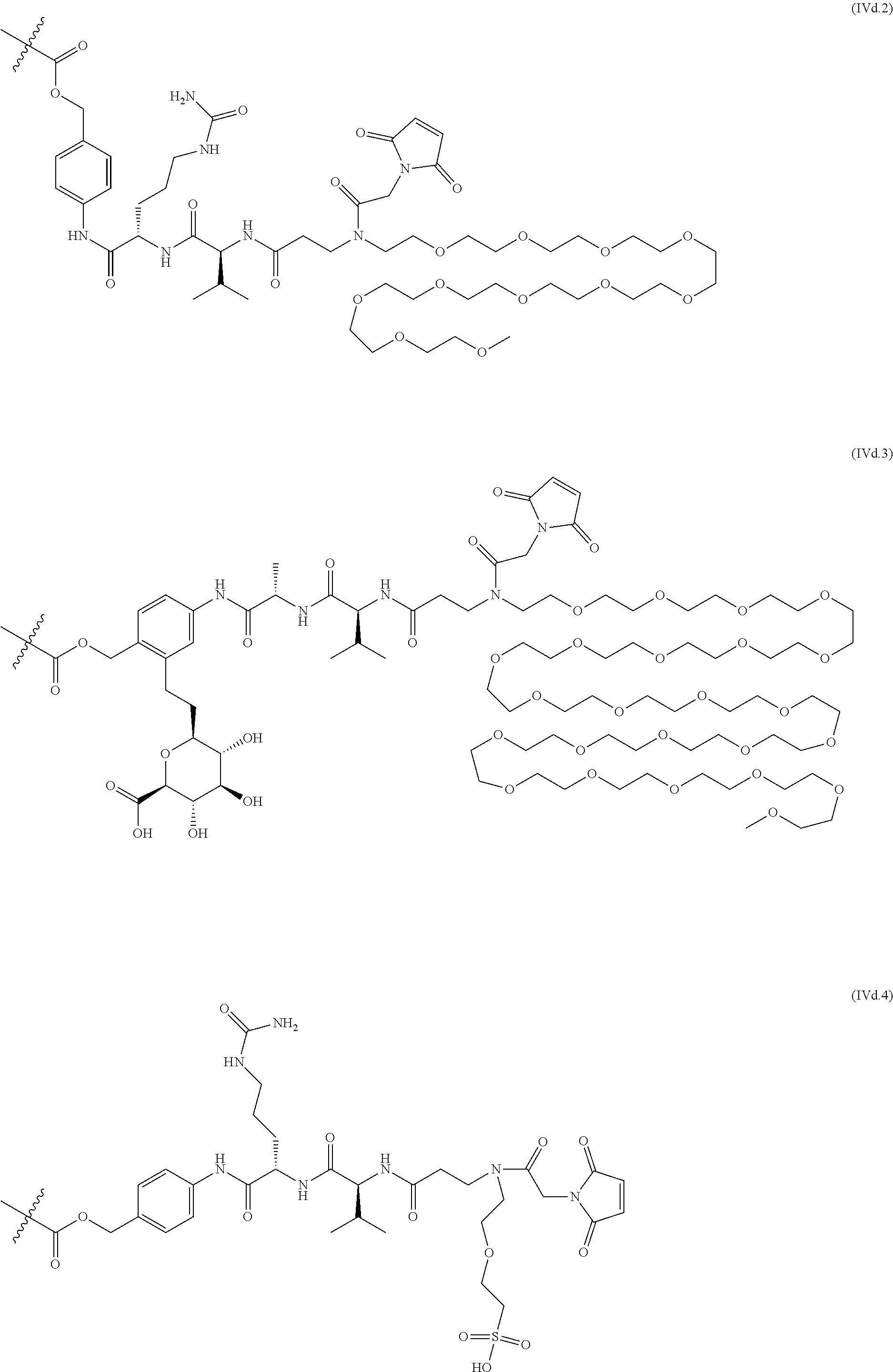 [SMILES: *C(=O)OCc1ccc(NC(=O)[C@H](CCCNC(N)=O)NC(=O)[C@@H](NC(=O)CCN(CCOCCOCCOCCOCCOCCOCCOCCOCCOCCOCCOC)C(=O)CN2C(=O)C=CC2=O)C(C)C)cc1 ; *C(=O)OCc1ccc(NC(=O)[C@H](C)NC(=O)[C@@H](NC(=O)CCN(CCOCCOCCOCCOCCOCCOCCOCCOCCOCCOCCOCCOCCOCCOCCOCCOCCOCCOCCOCCOCCOCCOCCOCCOC)C(=O)CN2C(=O)C=CC2=O)C(C)C)cc1CC[C@@H]1O[C@H](C(=O)O)[C@@H](O)[C@H](O)[C@H]1O ; *C(=O)OCc1ccc(NC(=O)[C@H](CCCNC(N)=O)NC(=O)[C@@H](NC(=O)CCN(CCOCCS(=O)(=O)O)C(=O)CN2C(=O)C=CC2=O)C(C)C)cc1]

In certain embodiments, the linker comprises an enzymatically cleavable sugar moiety, for example, a linker comprising structural formula (Va), (Vb), (Vc), (Vd), or (Ve):

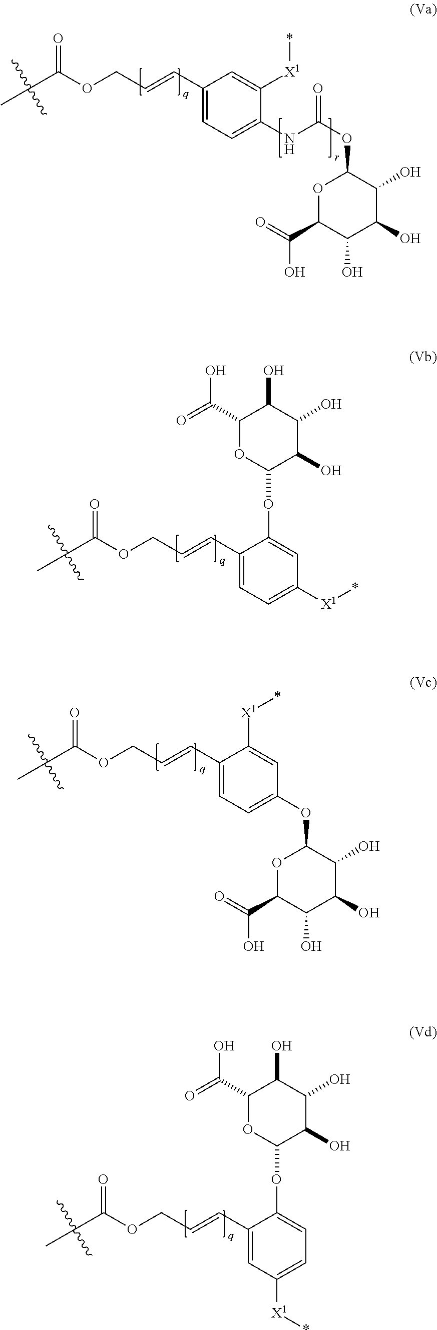

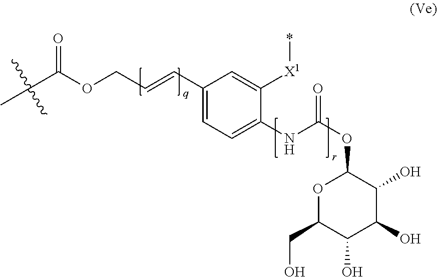

or a salt thereof, wherein:
q is 0 or 1;
r is 0 or 1;
X¹ is CH₂, O or NH;
[symbol] represents the point of attachment of the linker to the drug; and
* represents the point of attachment to the remainder of the linker.

Exemplary embodiments of linkers according to structural formula (Va) that may be included in the ADCs described herein include the linkers illustrated below (as illustrated, the linkers include a group suitable for covalently linking the linker to an antibody):

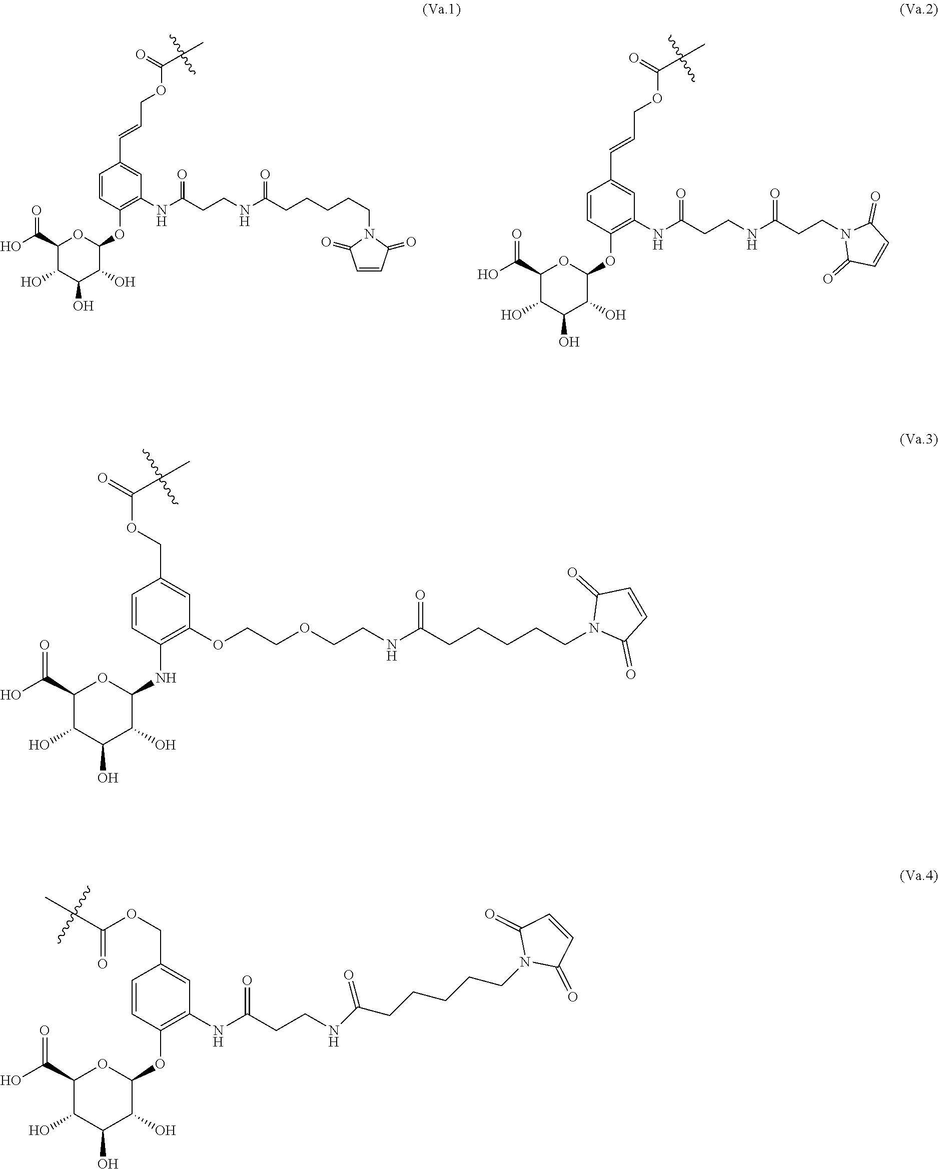 [SMILES: *C(=O)OC/C=C/c1ccc(O[C@@H]2O[C@H](C(=O)O)[C@@H](O)[C@H](O)[C@H]2O)c(NC(=O)CCNC(=O)CCCCCN2C(=O)C=CC2=O)c1 ; *C(=O)OC/C=C/c1ccc(O[C@@H]2O[C@H](C(=O)O)[C@@H](O)[C@H](O)[C@H]2O)c(NC(=O)CCNC(=O)CCN2C(=O)C=CC2=O)c1 ; *C(=O)OCc1ccc(N[C@@H]2O[C@H](C(=O)O)[C@@H](O)[C@H](O)[C@H]2O)c(OCCOCCNC(=O)CCCCCN2C(=O)C=CC2=O)c1 ; *C(=O)OCc1ccc(O[C@@H]2O[C@H](C(=O)O)[C@@H](O)[C@H](O)[C@H]2O)c(NC(=O)CCNC(=O)CCCCCN2C(=O)C=CC2=O)c1]

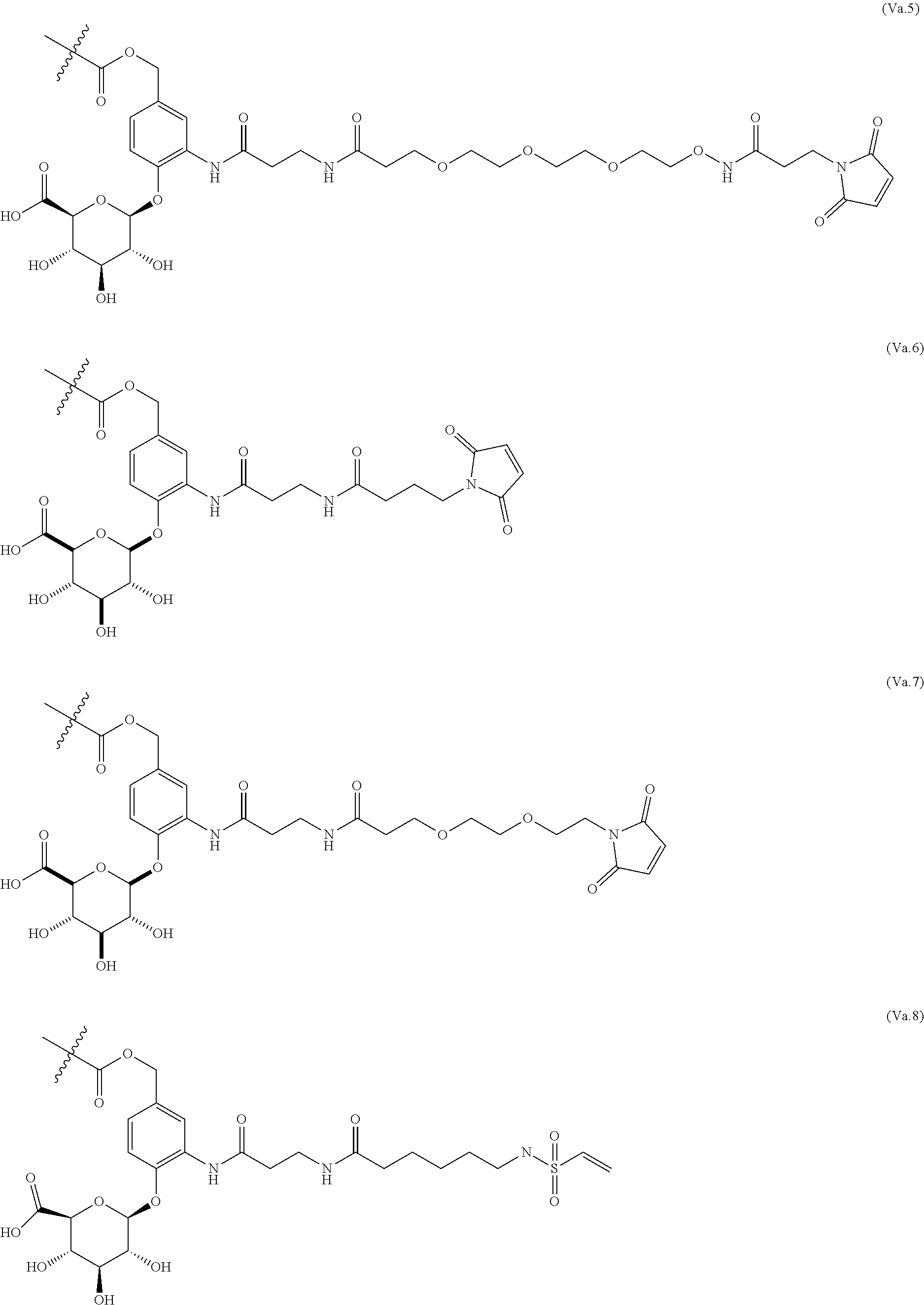

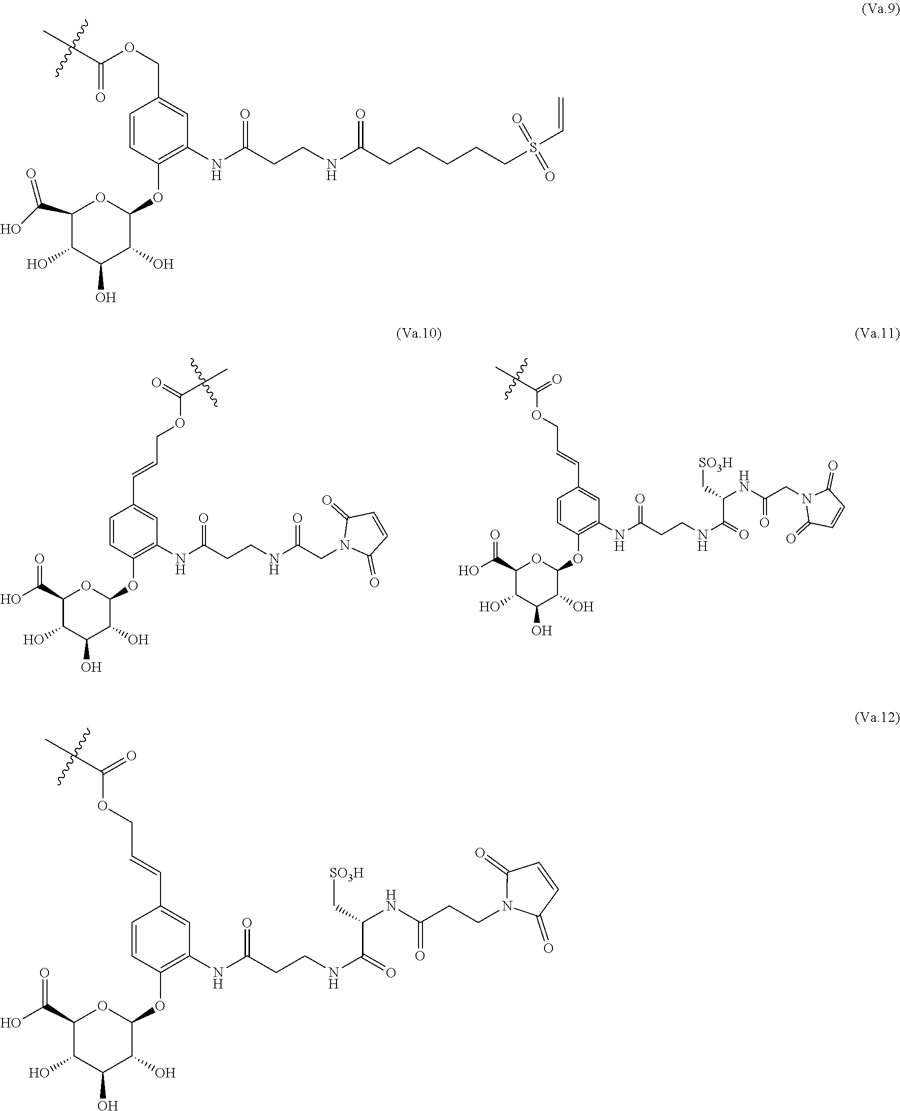 [SMILES: *C(=O)OCc1ccc(O[C@@H]2O[C@H](C(=O)O)[C@@H](O)[C@H](O)[C@H]2O)c(NC(=O)CCNC(=O)CCCCCS(=O)(=O)C=C)c1 ; *C(=O)OC/C=C/c1ccc(O[C@@H]2O[C@H](C(=O)O)[C@@H](O)[C@H](O)[C@H]2O)c(NC(=O)CCNC(=O)CN2C(=O)C=CC2=O)c1 ; *C(=O)OC/C=C/c1ccc(O[C@@H]2O[C@H](C(=O)O)[C@@H](O)[C@H](O)[C@H]2O)c(NC(=O)CCNC(=O)[C@H](CS(=O)(=O)O)NC(=O)CN2C(=O)C=CC2=O)c1 ; *C(=O)OC/C=C/c1ccc(O[C@@H]2O[C@H](C(=O)O)[C@@H](O)[C@H](O)[C@H]2O)c(NC(=O)CCNC(=O)[C@H](CS(=O)(=O)O)NC(=O)CCN2C(=O)C=CC2=O)c1]

Exemplary embodiments of linkers according to structural formula (Vb) that may be included in the ADCs described herein include the linkers illustrated below (as illustrated, the linkers include a group suitable for covalently linking the linker to an antibody):

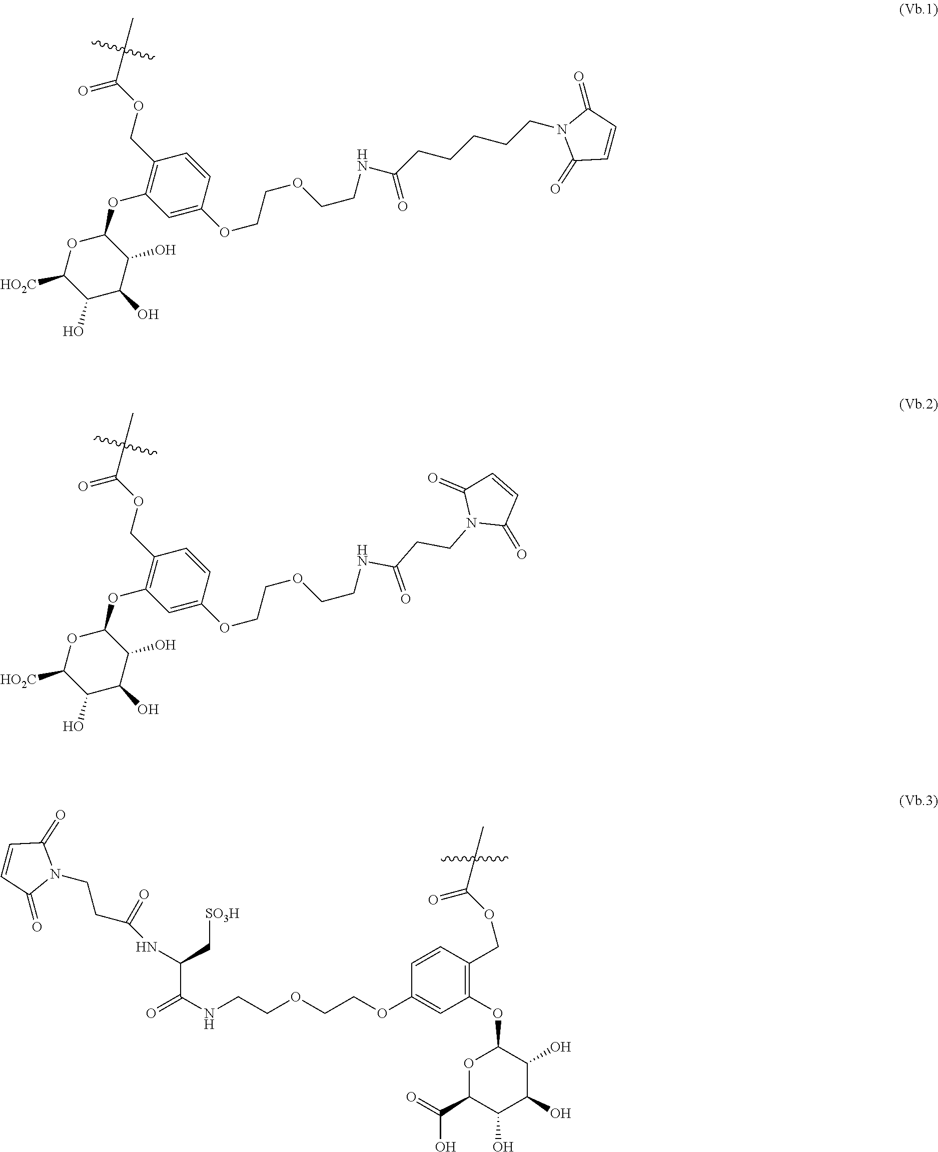 [SMILES: *C(=O)OCc1ccc(OCCOCCNC(=O)CCCCCN2C(=O)C=CC2=O)cc1O[C@@H]1O[C@H](C(=O)O)[C@@H](O)[C@H](O)[C@H]1O ; *C(=O)OCc1ccc(OCCOCCNC(=O)CCN2C(=O)C=CC2=O)cc1O[C@@H]1O[C@H](C(=O)O)[C@@H](O)[C@H](O)[C@H]1O ; *C(=O)OCc1ccc(OCCOCCNC(=O)[C@H](CS(=O)(=O)O)NC(=O)CCN2C(=O)C=CC2=O)cc1O[C@@H]1O[C@H](C(=O)O)[C@@H](O)[C@H](O)[C@H]1O]

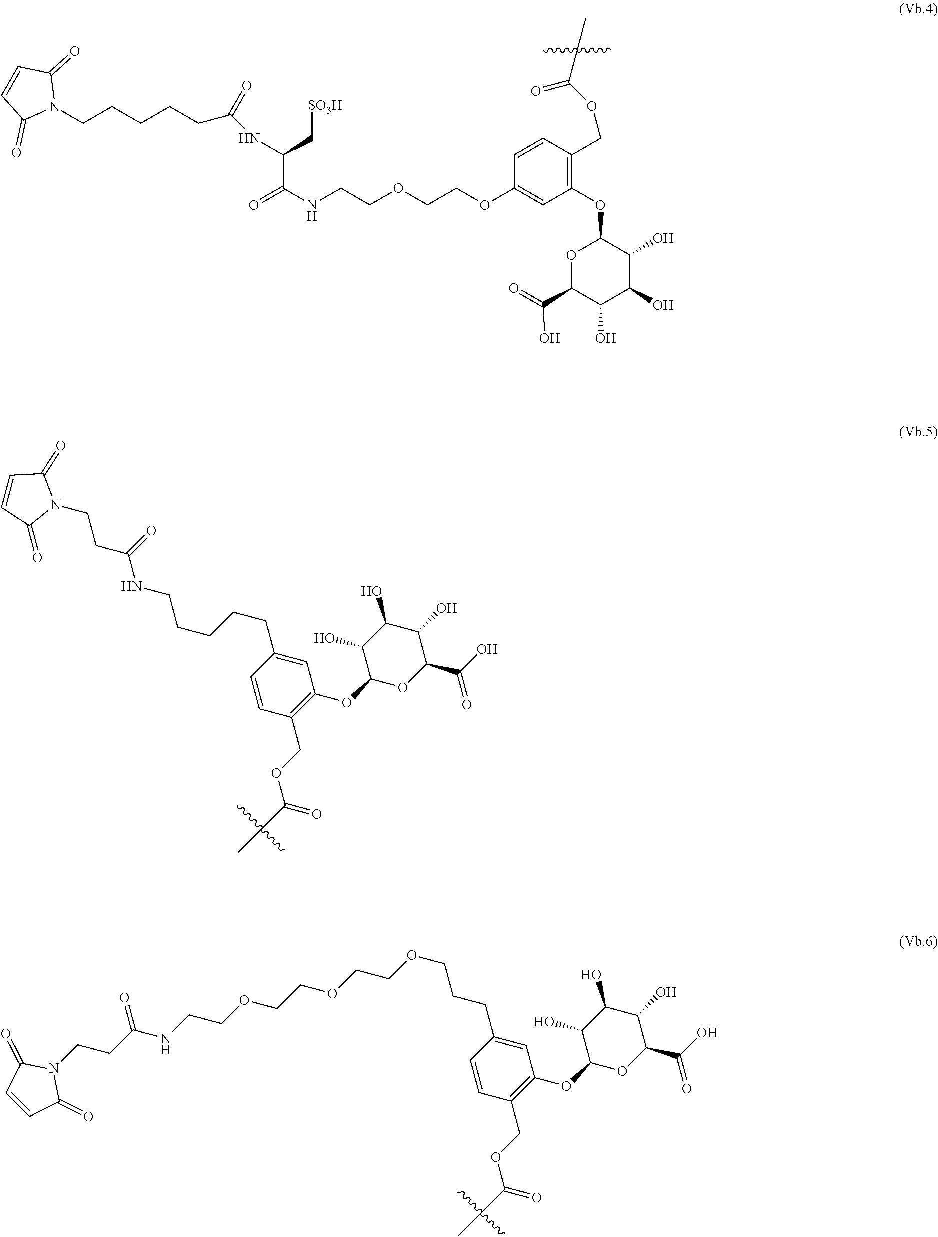 [SMILES: *C(=O)OCc1ccc(OCCOCCNC(=O)[C@H](CS(=O)(=O)O)NC(=O)CCCCCN2C(=O)C=CC2=O)cc1O[C@@H]1O[C@H](C(=O)O)[C@@H](O)[C@H](O)[C@H]1O ; *C(=O)OCc1ccc(CCCCCNC(=O)CCN2C(=O)C=CC2=O)cc1O[C@@H]1O[C@H](C(=O)O)[C@@H](O)[C@H](O)[C@H]1O ; *C(=O)OCc1ccc(CCCOCCOCCOCCNC(=O)CCN2C(=O)C=CC2=O)cc1O[C@@H]1O[C@H](C(=O)O)[C@@H](O)[C@H](O)[C@H]1O]

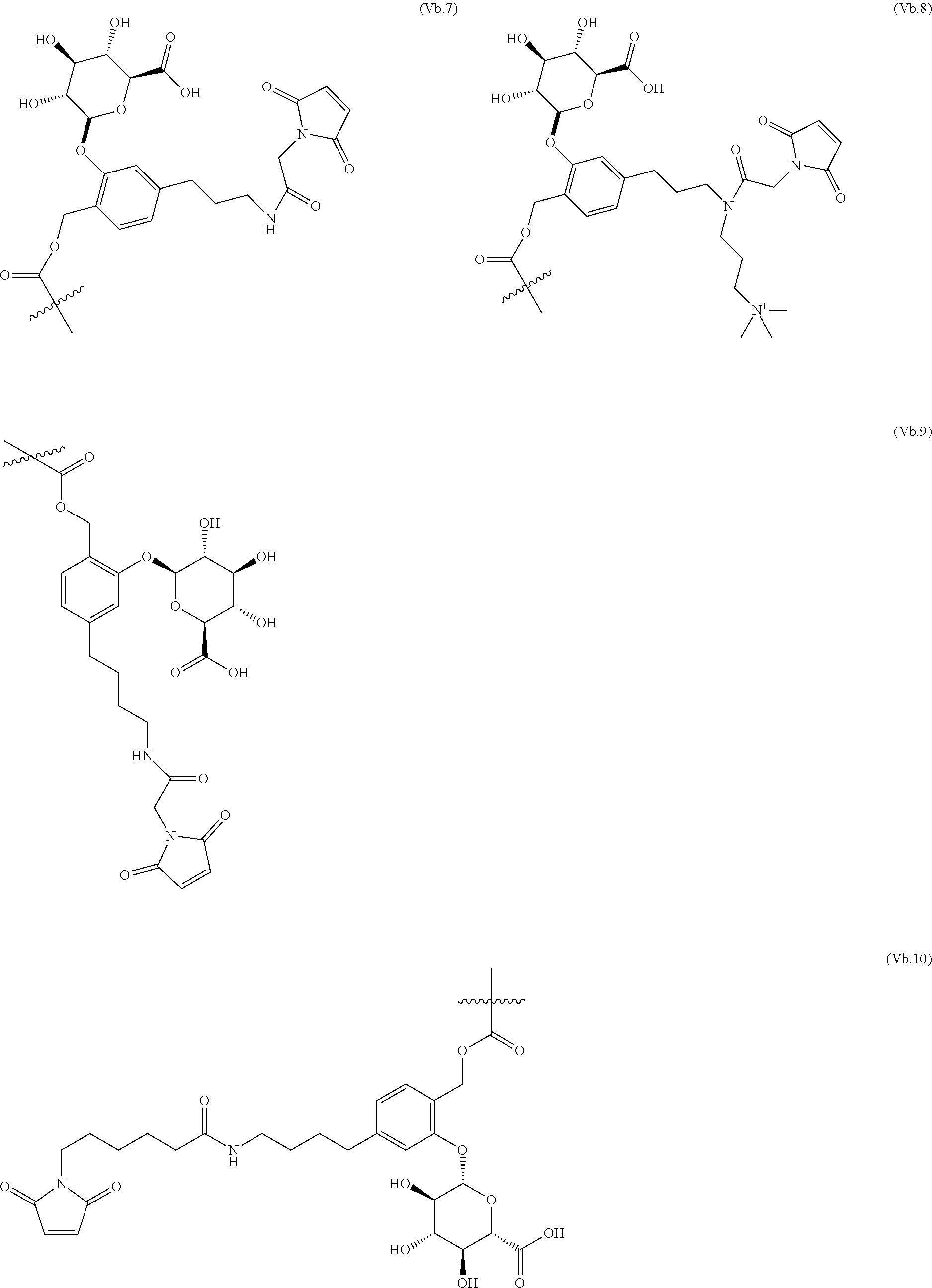

Exemplary embodiments of linkers according to structural formula (Vc) that may be included in the ADCs described herein include the linkers illustrated below (as illustrated, the linkers include a group suitable for covalently linking the linker to an antibody):

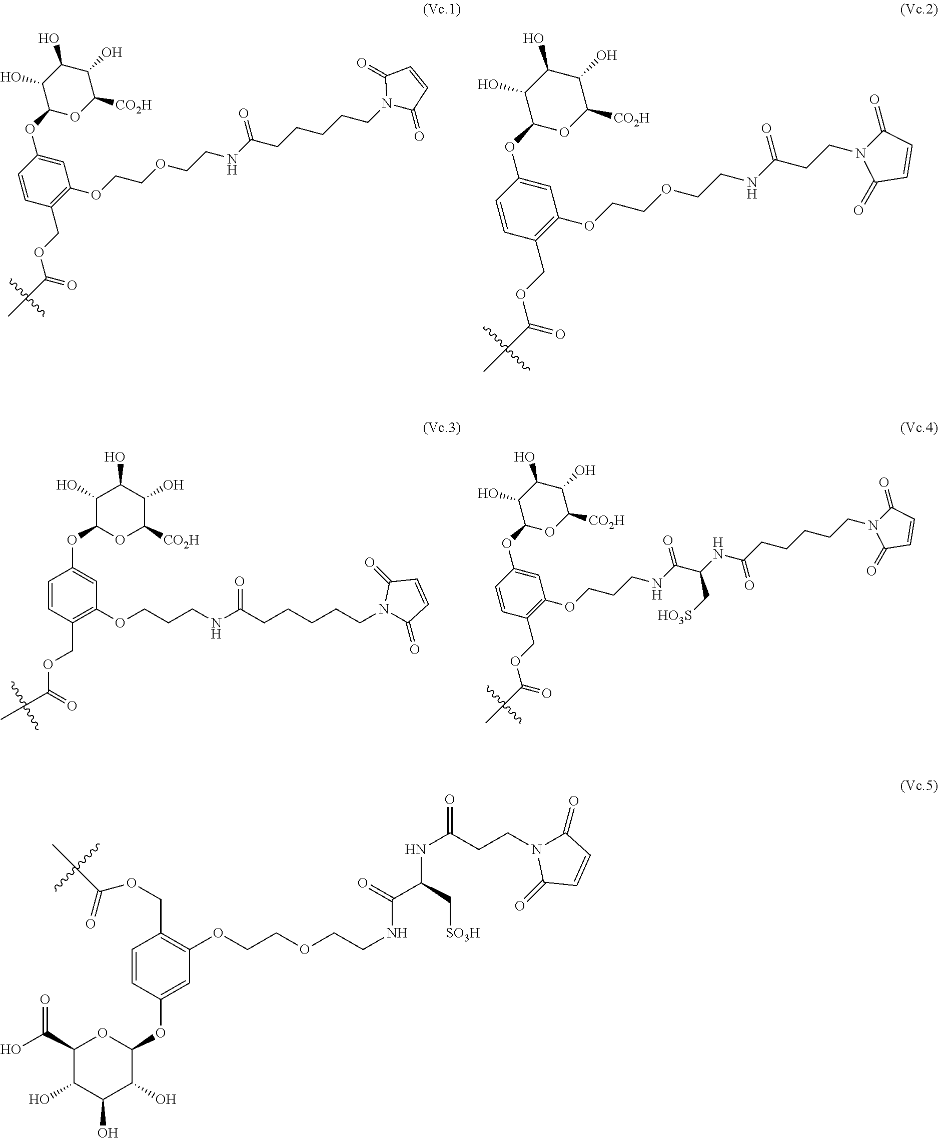

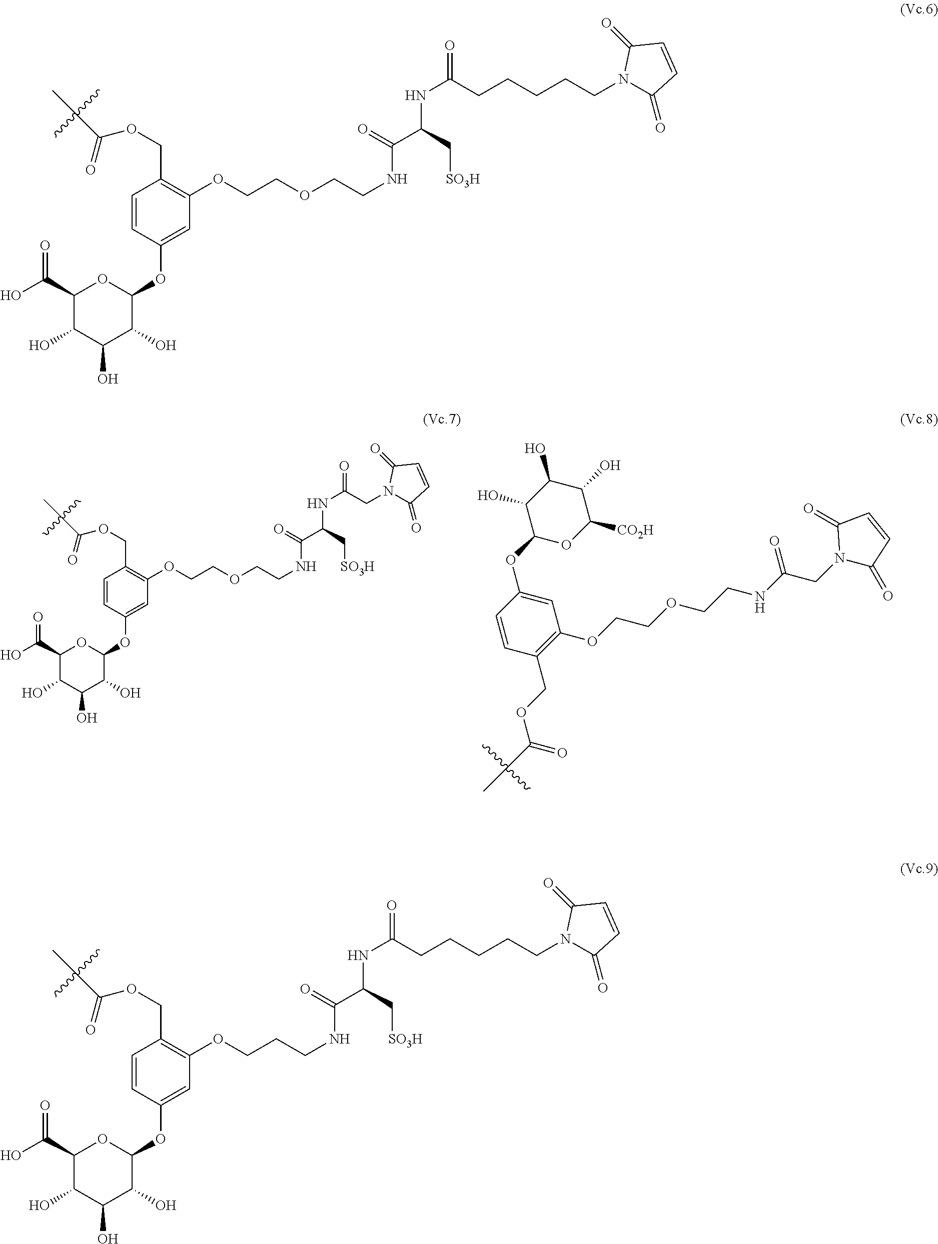 [SMILES: *C(=O)OCc1ccc(O[C@@H]2O[C@H](C(=O)O)[C@@H](O)[C@H](O)[C@H]2O)cc1OCCOCCNC(=O)[C@H](CS(=O)(=O)O)NC(=O)CCCCCN1C(=O)C=CC1=O ; *C(=O)OCc1ccc(O[C@@H]2O[C@H](C(=O)O)[C@@H](O)[C@H](O)[C@H]2O)cc1OCCOCCNC(=O)[C@H](CS(=O)(=O)O)NC(=O)CN1C(=O)C=CC1=O ; *C(=O)OCc1ccc(O[C@@H]2O[C@H](C(=O)O)[C@@H](O)[C@H](O)[C@H]2O)cc1OCCOCCNC(=O)CN1C(=O)C=CC1=O ; *C(=O)OCc1ccc(O[C@@H]2O[C@H](C(=O)O)[C@@H](O)[C@H](O)[C@H]2O)cc1OCCCNC(=O)[C@H](CS(=O)(=O)O)NC(=O)CCCCCN1C(=O)C=CC1=O]

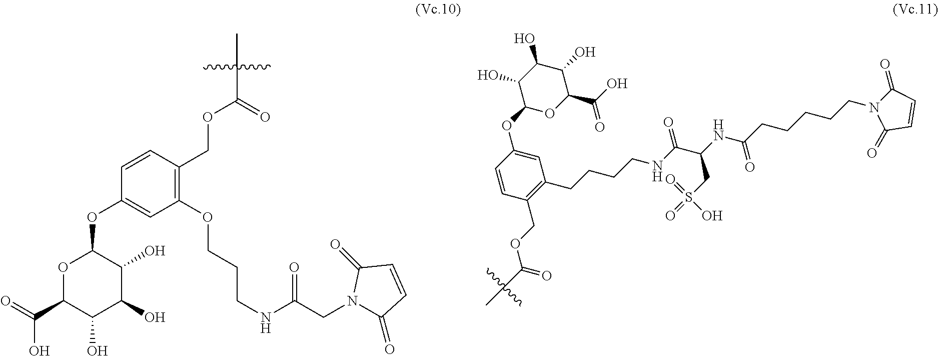 [SMILES: *C(=O)OCc1ccc(O[C@@H]2O[C@H](C(=O)O)[C@@H](O)[C@H](O)[C@H]2O)cc1OCCCNC(=O)CN1C(=O)C=CC1=O ; *C(=O)OCc1ccc(O[C@@H]2O[C@H](C(=O)O)[C@@H](O)[C@H](O)[C@H]2O)cc1CCCCNC(=O)[C@H](CS(=O)(=O)O)NC(=O)CCCCCN1C(=O)C=CC1=O]

Exemplary embodiments of linkers according to structural formula (Vd) that may be included in the ADCs described herein include the linkers illustrated below (as illustrated, the linkers include a group suitable for covalently linking the linker to an antibody):

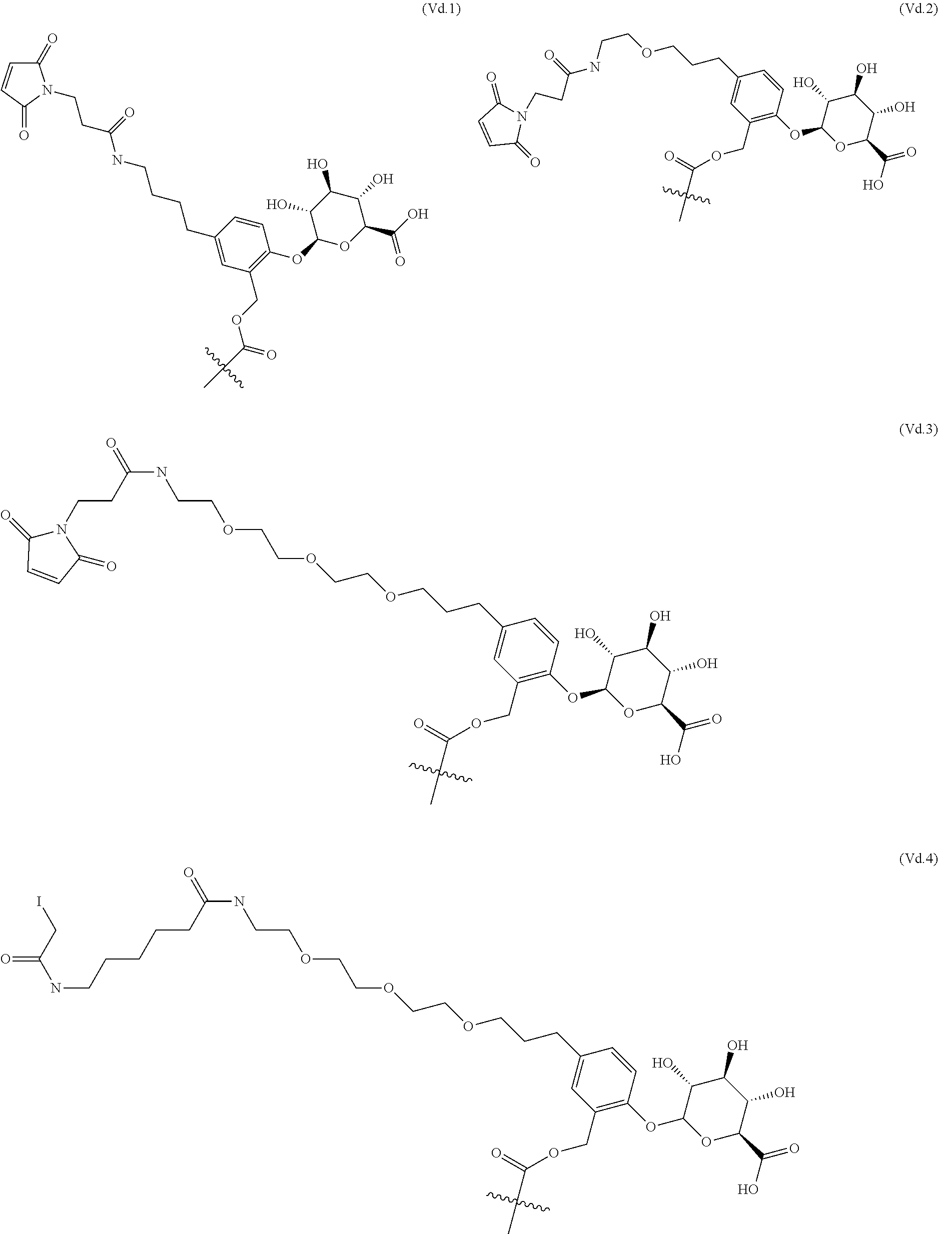 [SMILES: *C(=O)OCc1cc(CCCCNC(=O)CCN2C(=O)C=CC2=O)ccc1O[C@@H]1O[C@H](C(=O)O)[C@@H](O)[C@H](O)[C@H]1O ; *C(=O)OCc1cc(CCCOCCNC(=O)CCN2C(=O)C=CC2=O)ccc1O[C@@H]1O[C@H](C(=O)O)[C@@H](O)[C@H](O)[C@H]1O ; *C(=O)OCc1cc(CCCOCCOCCOCCNC(=O)CCN2C(=O)C=CC2=O)ccc1O[C@@H]1O[C@H](C(=O)O)[C@@H](O)[C@H](O)[C@H]1O ; *C(=O)OCc1cc(CCCOCCOCCOCCNC(=O)CCCCCNC(=O)CI)ccc1OC1O[C@H](C(=O)O)[C@@H](O)[C@H](O)[C@H]1O]

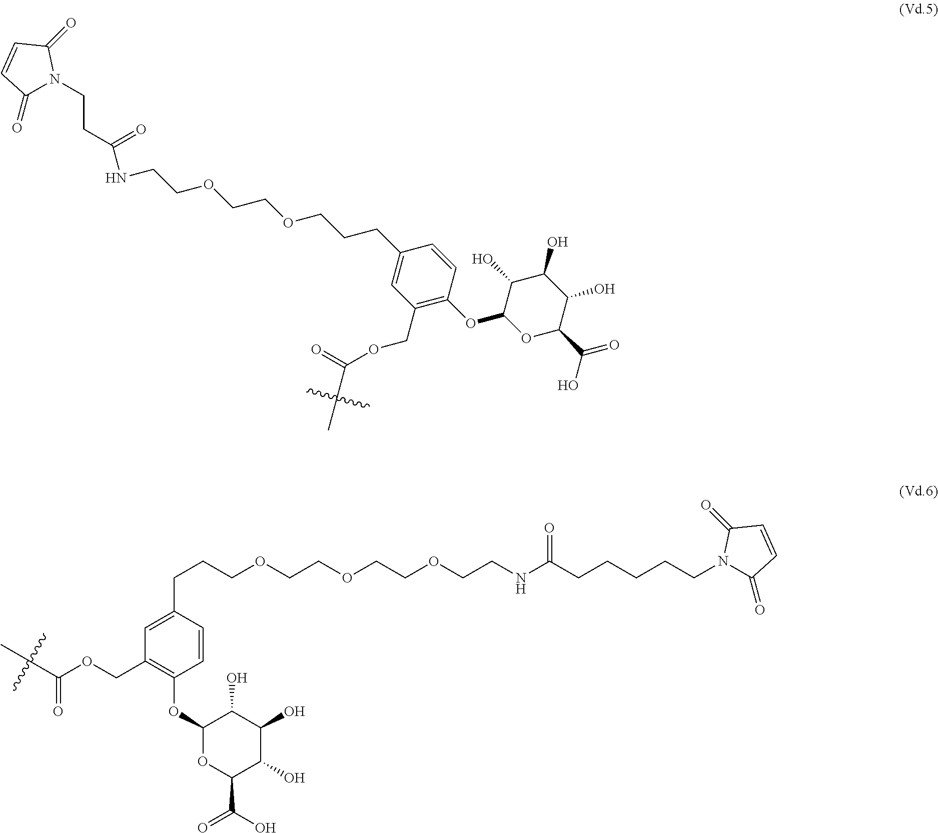

Exemplary embodiments of linkers according to structural formula (Ve) that may be included in the ADCs described herein include the linkers illustrated below (as illustrated, the linkers include a group suitable for covalently linking the linker to an antibody):

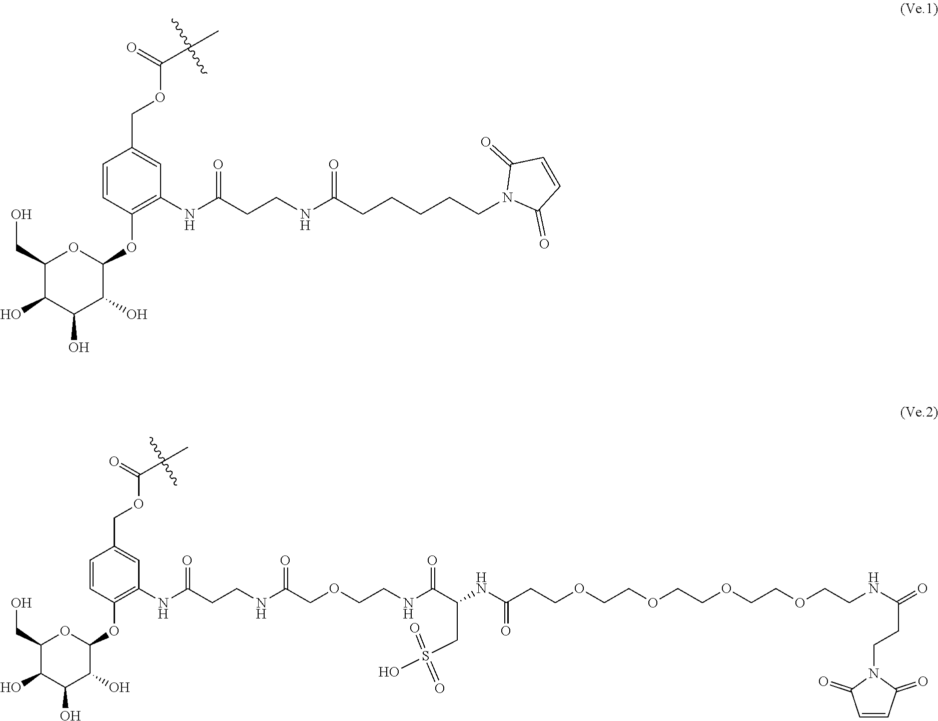 [SMILES: *C(=O)OCc1ccc(O[C@@H]2O[C@H](CO)[C@H](O)[C@H](O)[C@H]2O)c(NC(=O)CCNC(=O)CCCCCN2C(=O)C=CC2=O)c1 ; *C(=O)OCc1ccc(O[C@@H]2O[C@H](CO)[C@H](O)[C@H](O)[C@H]2O)c(NC(=O)CCNC(=O)COCCNC(=O)[C@@H](CS(=O)(=O)O)NC(=O)CCOCCOCCOCCOCCNC(=O)CCN2C(=O)C=CC2=O)c1]

### Non-Cleavable Linkers

Although cleavable linkers may provide certain advantages, the linkers comprising the ADC described herein need not be cleavable. For noncleavable linkers, the drug release does not depend on the differential properties between the plasma and some cytoplasmic compartments. The release of the drug is postulated to occur after internalization of the ADC via antigen-mediated endocytosis and delivery to lysosomal compartment, where the antibody is degraded to the level of amino acids through intracellular proteolytic degradation. This process releases a drug derivative, which is formed by the drug, the linker, and the amino acid residue to which the linker was covalently attached. The amino-acid drug metabolites from conjugates with noncleavable linkers are more hydrophilic and generally less membrane permeable, which leads to less bystander effects and less nonspecific toxicities compared to conjugates with a cleavable linker. In general, ADCs with noncleavable linkers have greater stability in circulation than ADCs with cleavable linkers. Non-cleavable linkers may be alkylene chains, or maybe polymeric in natures, such as, for example, based upon polyalkylene glycol polymers, amide polymers, or may include segments of alkylene chains, polyalkylene glycols and/or amide polymers. In certain embodiments, the linker comprises a polyethylene glycol segment having from 1 to 6 ethylene glycol units.

A variety of non-cleavable linkers used to link drugs to antibodies have been described. (See, Jeffrey et al., 2006, Bioconjug. Chem. 17; 831-840; Jeffrey et al., 2007, Bioorg. Med. Chem. Lett. 17:2278-2280; and Jiang et al., 2005, J. Am. Chem. Soc. 127:11254-11255, the contents of which are incorporated herein by reference). All of these linkers may be included in the ADCs described herein.

In certain embodiments, the linker is non-cleavable in vivo, for example a linker according to structural formula (VIa), (VIb), (VIc) or (VId) (as illustrated, the linkers include a group suitable for covalently linking the linker to an antibody:

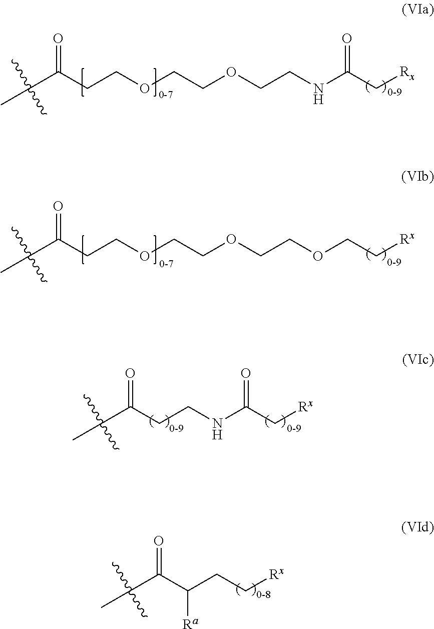

or salts thereof, wherein:
R^{a} is selected from hydrogen, alkyl, sulfonate and methyl sulfonate;
R^{x} is a moiety including a functional group capable of covalently linking the linker to an antibody; and
[symbol] represents the point of attachment of the linker to the Bcl-xL inhibitor.

Exemplary embodiments of linkers according to structural formula (VIa)-(VId) that may be included in the ADCs described herein include the linkers illustrated below (as illustrated, the linkers include a group suitable for covalently linking the linker to an antibody, and “ [symbol] ” represents the point of attachment to a Bcl-xL inhibitor):

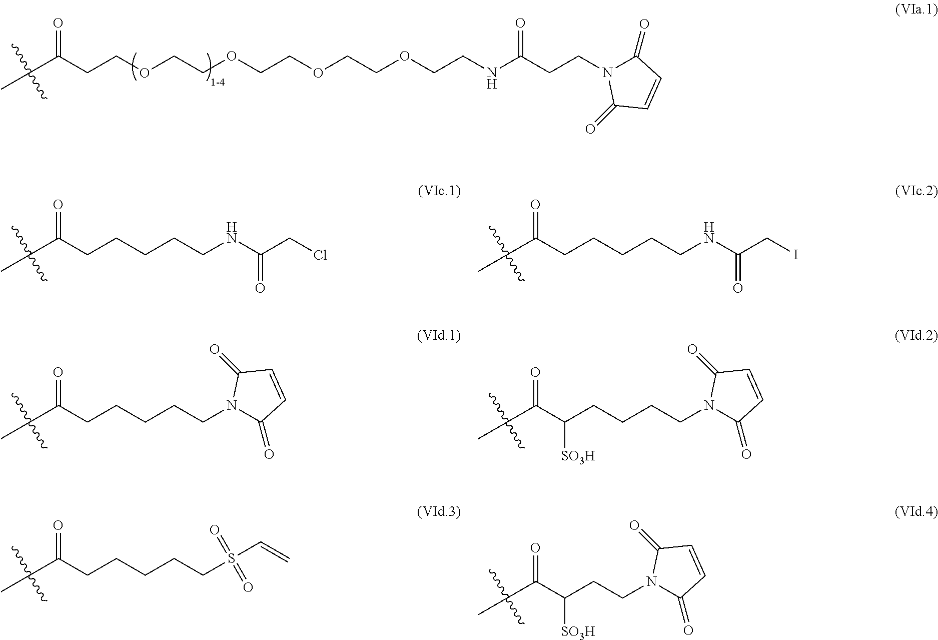

### 4.4.1.2. Groups Used to Attach Linkers to Antibodies

Attachment groups can be electrophilic in nature and include: maleimide groups, activated disulfides, active esters such as NHS esters and HOBt esters, haloformates, acid halides, alkyl and benzyl halides such as haloacetamides. As discussed below, there are also emerging technologies related to “self-stabilizing” maleimides and “bridging disulfides” that can be used in accordance with the disclosure.

Loss of the drug-linker from the ADC has been observed as a result of a maleimide exchange process with albumin, cysteine or glutathione (Alley et al., 2008, Bioconjugate Chem. 19: 759-769). This is particularly prevalent from highly solvent-accessible sites of conjugation while sites that are partially accessible and have a positively charged environment promote maleimide ring hydrolysis (Junutula et al., 2008, Nat. Biotechnol. 26: 925-932). A recognized solution is to hydrolyze the succinimide formed from conjugation as this is resistant to deconjugation from the antibody, thereby making the ADC stable in serum. It has been reported previously that the succinimide ring will undergo hydrolysis under alkaline conditions (Kalia et al., 2007, Bioorg. Med. Chem. Lett. 17: 6286-6289). One example of a “self-stabilizing” maleimide group that hydrolyzes spontaneously under antibody conjugation conditions to give an ADC species with improved stability is depicted in the schematic below. See U.S. Published Application No. 2013/0309256 and Lyon et al., 2014, Nat. Biotechnol. 32: 1059-1062. Thus, the maleimide attachment group is reacted with a sulfhydryl of an antibody to give an intermediate succinimide ring. The hydrolyzed form of the attachment group is resistant to deconjugation in the presence of plasma proteins.

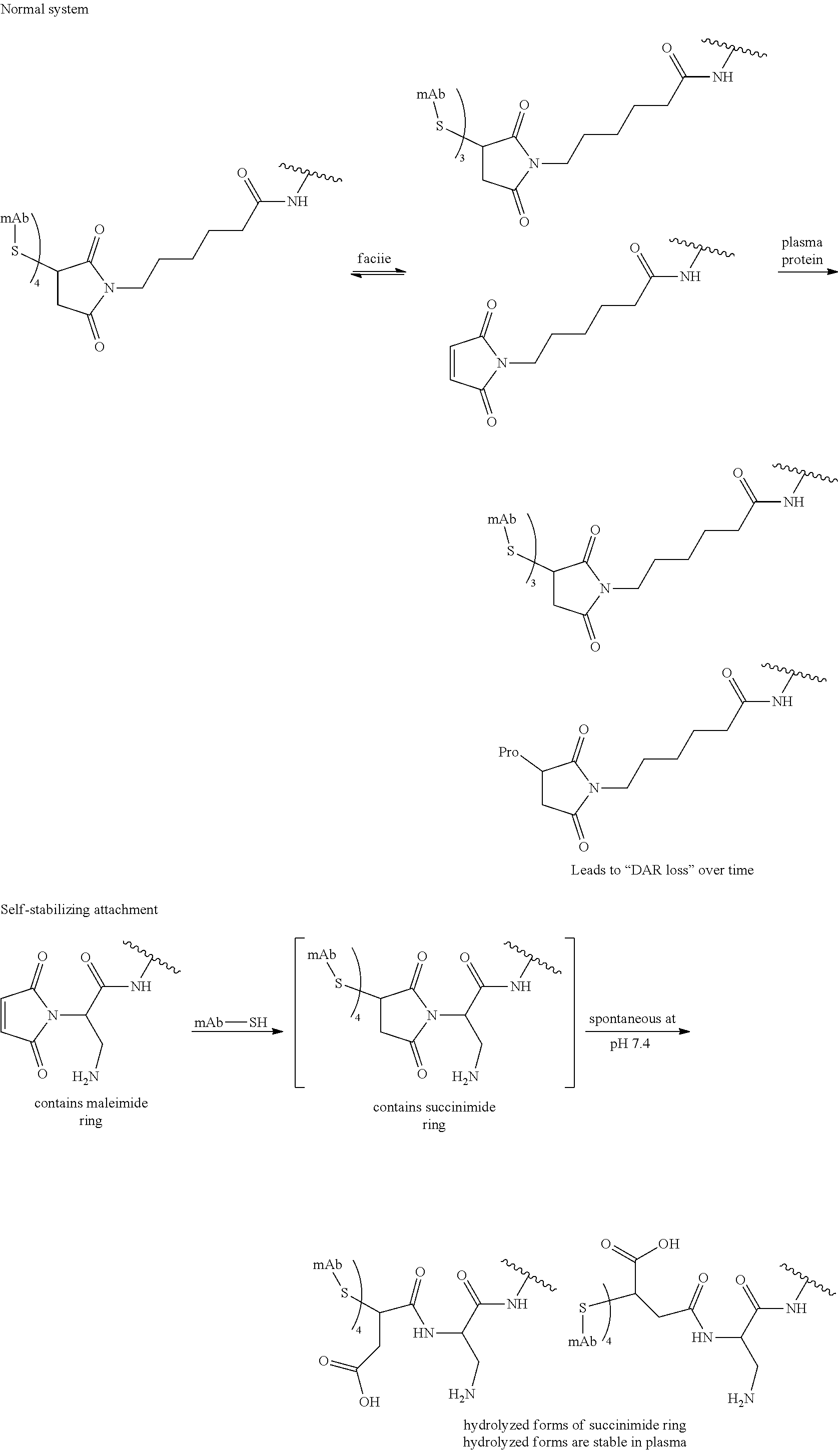

Polytherics has disclosed a method for bridging a pair of sulfhydryl groups derived from reduction of a native hinge disulfide bond. See, Badescu et al., 2014, Bioconjugate Chem. 25:1124-1136. The reaction is depicted in the schematic below. An advantage of this methodology is the ability to synthesize homogenous DAR4 ADCs by full reduction of IgGs (to give 4 pairs of sulfhydryls) followed by reaction with 4 equivalents of the alkylating agent. ADCs containing “bridged disulfides” are also claimed to have increased stability.

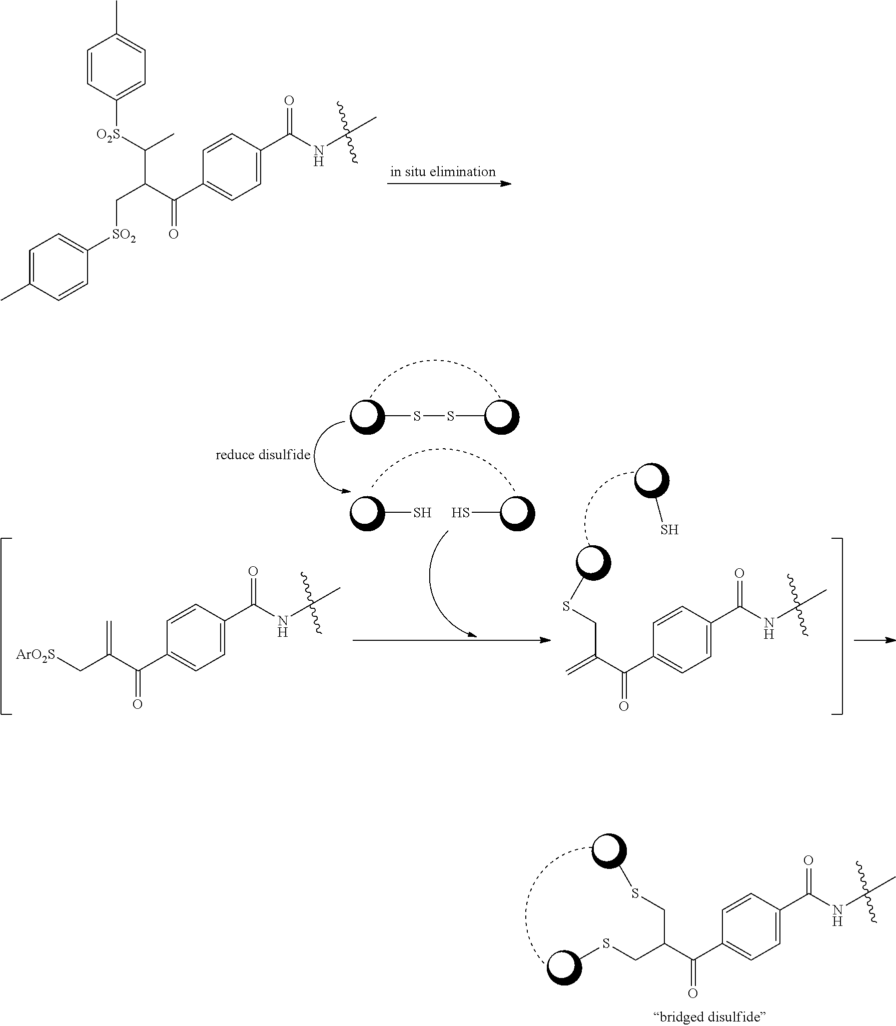

Similarly, as depicted below, a maleimide derivative that is capable of bridging a pair of sulfhydryl groups has been developed. See U.S. Published Application No. 2013/0224228.

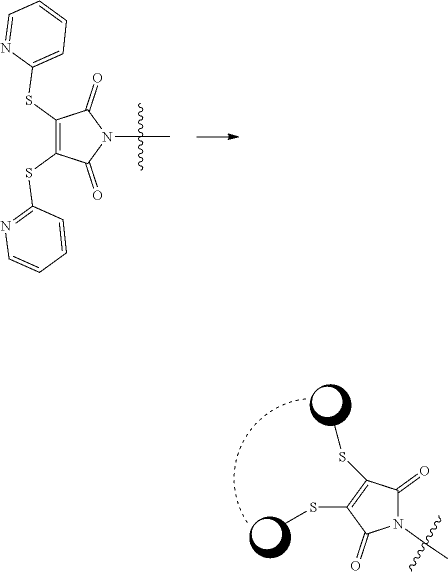 [SMILES: *N1C(=O)C(Sc2ccccn2)=C(Sc2ccccn2)C1=O ; *N1C(=O)C(SC)=C(SC)C1=O]

In certain embodiments the attachment moiety comprises the structural formulae (VIIa), (VIIb), or (VIIc):

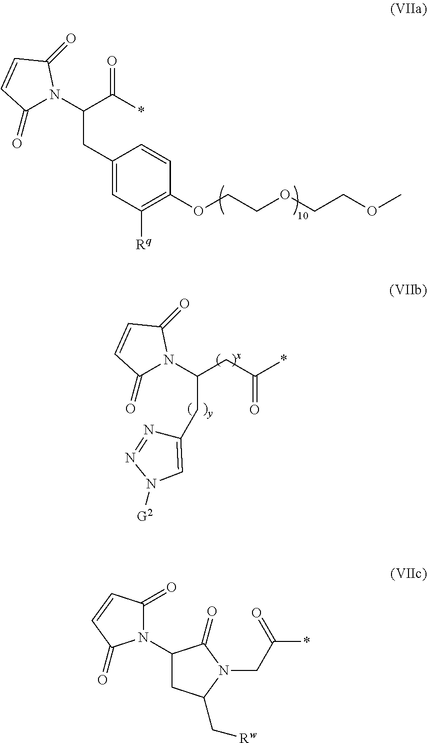

or salts thereof, wherein:
R^{q} is H or —O—(CH₂CH₂O)₁₁—CH₃;
x is 0 or 1;
y is 0 or 1;
G² is —CH₂CH₂CH₂SO₃H or —CH₂CH₂O—(CH₂CH₂O)₁₁—CH₃;
R^{w} is —O—CH₂CH₂SO₃H or —NH(CO)—CH₂CH₂O—(CH₂CH₂O)₁₂—CH₃; and
* represents the point of attachment to the remainder of the linker.

Exemplary embodiments of linkers according to structural formula (VIIa) and (VIIb) that may be included in the ADCs described herein include the linkers illustrated below (as illustrated, the linkers include a group suitable for covalently linking the linker to an antibody):

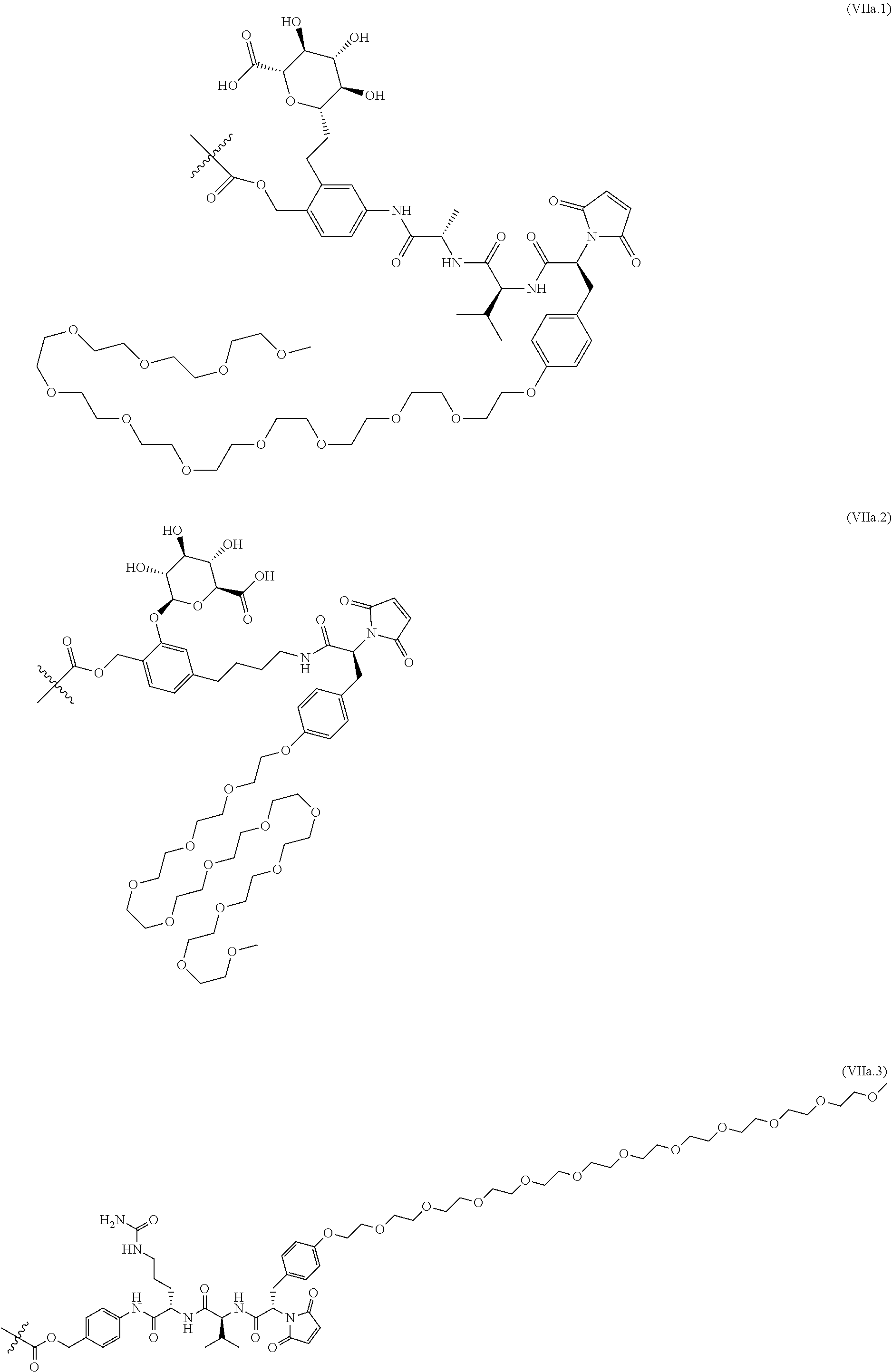 [SMILES: *C(=O)OCc1ccc(NC(=O)[C@H](C)NC(=O)[C@@H](NC(=O)[C@H](Cc2ccc(OCCOCCOCCOCCOCCOCCOCCOCCOCCOCCOCCOC)cc2)N2C(=O)C=CC2=O)C(C)C)cc1CC[C@@H]1O[C@H](C(=O)O)[C@@H](O)[C@H](O)[C@H]1O ; *C(=O)OCc1ccc(CCCCNC(=O)[C@H](Cc2ccc(OCCOCCOCCOCCOCCOCCOCCOCCOCCOCCOCCOC)cc2)N2C(=O)C=CC2=O)cc1O[C@@H]1O[C@H](C(=O)O)[C@@H](O)[C@H](O)[C@H]1O ; *C(=O)OCc1ccc(NC(=O)[C@H](CCCNC(N)=O)NC(=O)[C@@H](NC(=O)[C@H](Cc2ccc(OCCOCCOCCOCCOCCOCCOCCOCCOCCOCCOCCOC)cc2)N2C(=O)C=CC2=O)C(C)C)cc1]

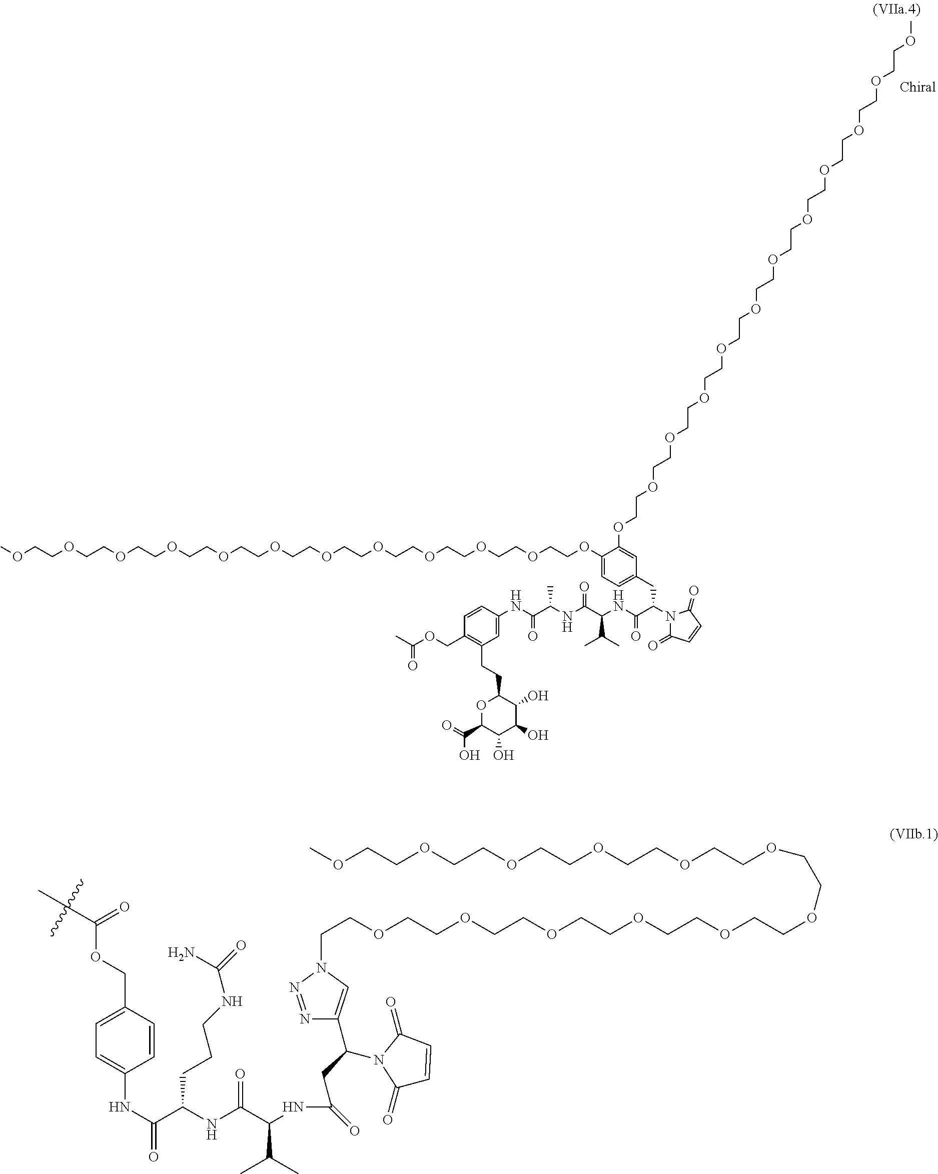

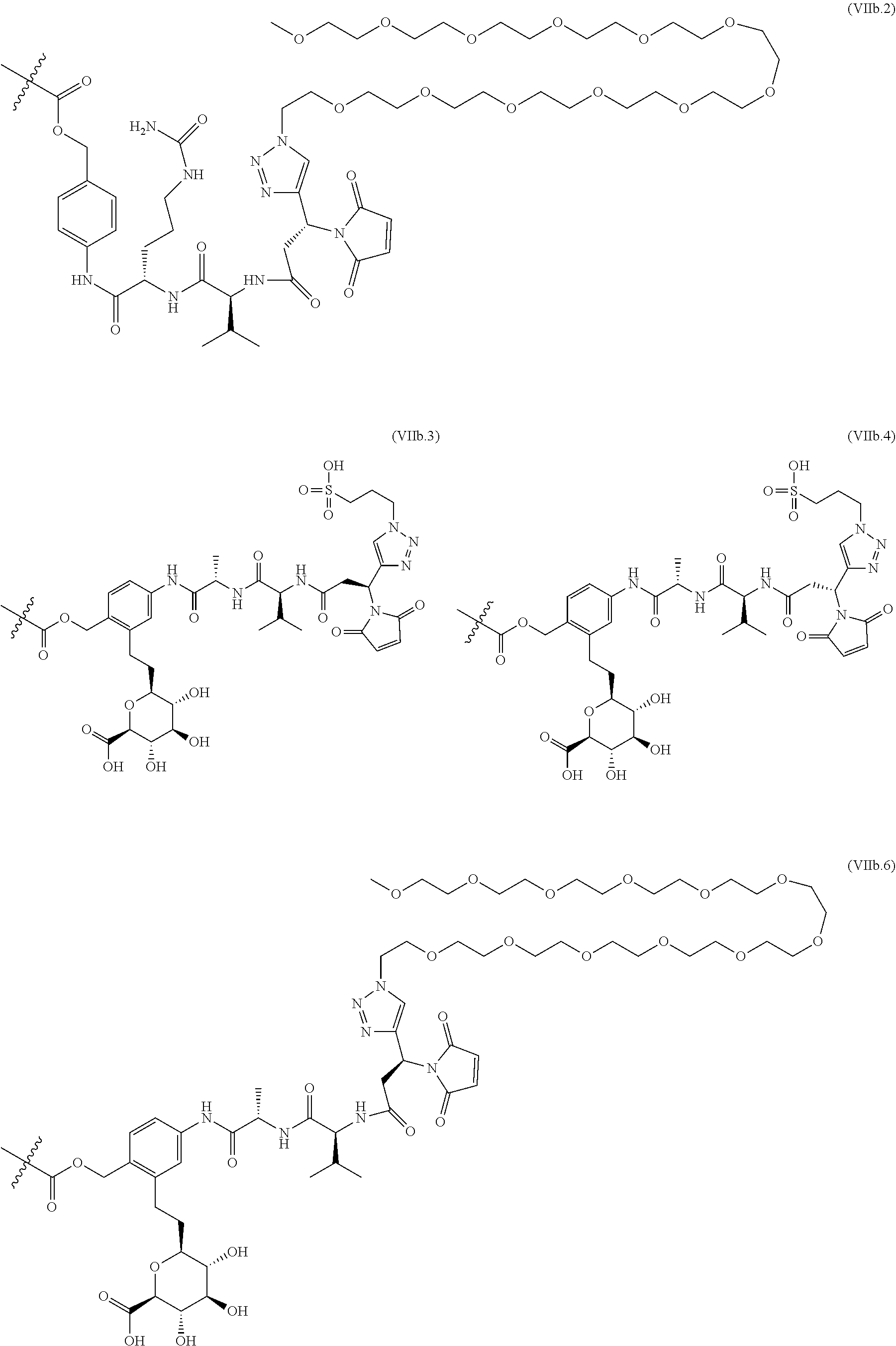

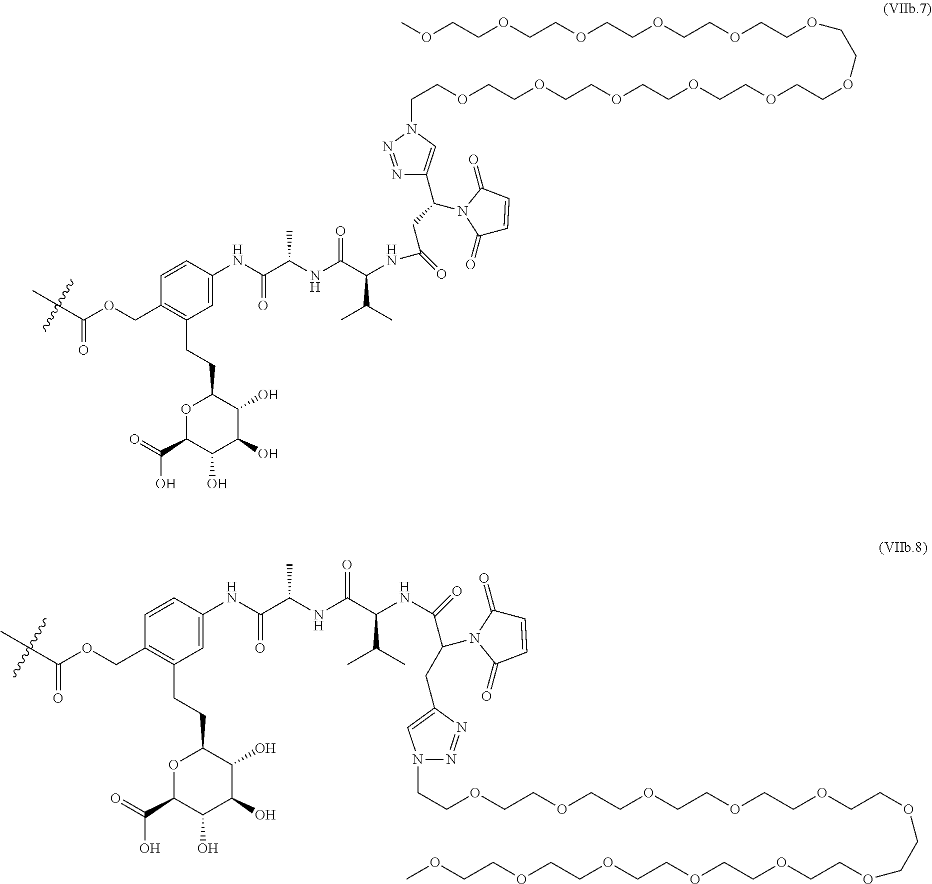 [SMILES: *C(=O)OCc1ccc(NC(=O)[C@H](C)NC(=O)[C@@H](NC(=O)C[C@H](c2cn(CCOCCOCCOCCOCCOCCOCCOCCOCCOCCOCCOCCOC)nn2)N2C(=O)C=CC2=O)C(C)C)cc1CC[C@@H]1O[C@H](C(=O)O)[C@@H](O)[C@H](O)[C@H]1O ; *C(=O)OCc1ccc(NC(=O)[C@H](C)NC(=O)[C@@H](NC(=O)C(Cc2cn(CCOCCOCCOCCOCCOCCOCCOCCOCCOCCOCCOCCOC)nn2)N2C(=O)C=CC2=O)C(C)C)cc1CC[C@@H]1O[C@H](C(=O)O)[C@@H](O)[C@H](O)[C@H]1O]

Exemplary embodiments of linkers according to structural formula (VIIc) that may be included in the ADCs described herein include the linkers illustrated below (as illustrated, the linkers include a group suitable for covalently linking the linker to an antibody):

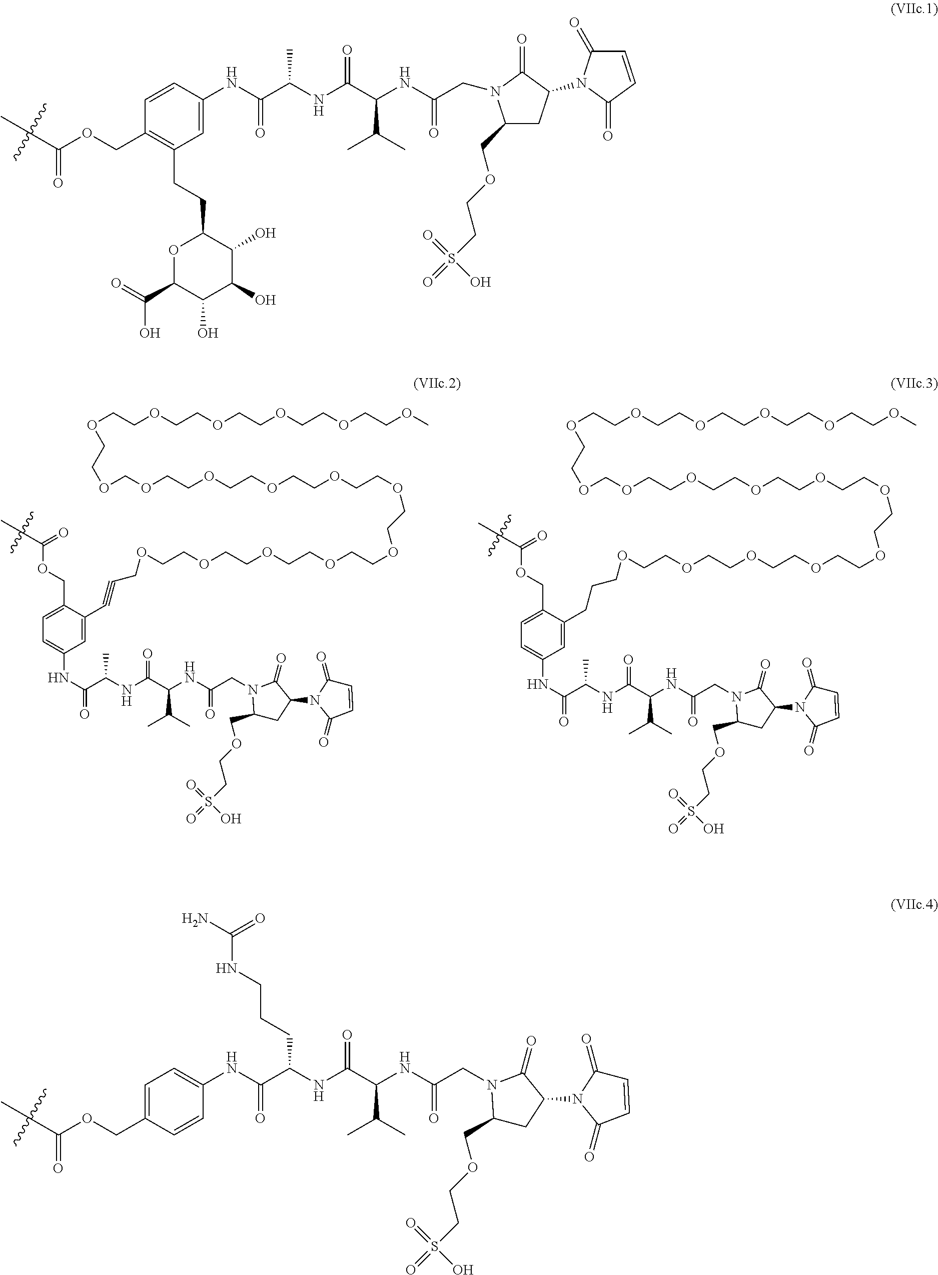

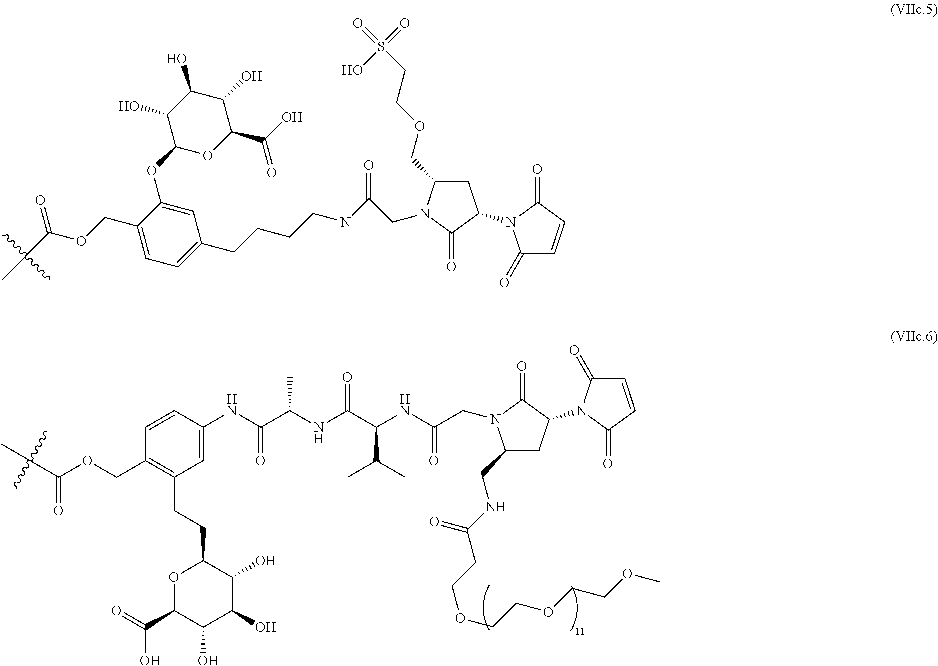

### 4.4.1.3. Linker Selection Considerations

As is known by skilled artisans, the linker selected for a particular ADC may be influenced by a variety of factors, including but not limited to, the site of attachment to the antibody (e.g., lys, cys or other amino acid residues), structural constraints of the drug pharmacophore and the lipophilicity of the drug. The specific linker selected for an ADC should seek to balance these different factors for the specific antibody/drug combination. For a review of the factors that are influenced by choice of linkers in ADCs, see Nolting, Chapter 5 “Linker Technology in Antibody-Drug Conjugates,” In: Antibody-Drug Conjugates: Methods in Molecular Biology, vol. 1045, pp. 71-100, Laurent Ducry (Ed.), Springer Science & Business Medica, LLC, 2013.

For example, ADCs have been observed to effect killing of bystander antigen-negative cells present in the vicinity of the antigen-positive tumor cells. The mechanism of bystander cell killing by ADCs has indicated that metabolic products formed during intracellular processing of the ADCs may play a role. Neutral cytotoxic metabolites generated by metabolism of the ADCs in antigen-positive cells appear to play a role in bystander cell killing while charged metabolites may be prevented from diffusing across the membrane into the medium and therefore cannot affect bystander killing. In certain embodiments, the linker is selected to attenuate the bystander killing effect caused by cellular metabolites of the ADC. In certain embodiments, the linker is selected to increase the bystander killing effect.

The properties of the linker may also impact aggregation of the ADC under conditions of use and/or storage. Typically, ADCs reported in the literature contain no more than 34 drug molecules per antibody molecule (see, e.g., Chari, 2008, Acc Chem Res 41:98-107). Attempts to obtain higher drug-to-antibody ratios (“DAR”) often failed, particularly if both the drug and the linker were hydrophobic, due to aggregation of the ADC (see King et al., 2002, J Med Chem 45:4336-4343; Hollander et al., 2008, Bioconjugate Chem 19:358-361; Burke et al., 2009 Bioconjugate Chem 20:1242-1250). In many instances, DARs higher than 3-4 could be beneficial as a means of increasing potency. In instances where the Bcl-xL inhibitor is hydrophobic in nature, it may be desirable to select linkers that are relatively hydrophilic as a means of reducing ADC aggregation, especially in instances where DARS greater than 3-4 are desired. Thus, in certain embodiments, the linker incorporates chemical moieties that reduce aggregation of the ADCs during storage and/or use. A linker may incorporate polar or hydrophilic groups such as charged groups or groups that become charged under physiological pH to reduce the aggregation of the ADCs. For example, a linker may incorporate charged groups such as salts or groups that deprotonate, e.g., carboxylates, or protonate, e.g., amines, at physiological pH.

Exemplary polyvalent linkers that have been reported to yield DARs as high as 20 that may be used to link numerous Bcl-xL inhibitors to an antibody are described in U.S. Pat. No. 8,399,512; U.S. Published Application No. 2010/0152725; U.S. Pat. Nos. 8,524,214; 8,349,308; U.S. Published Application No. 2013/189218; U.S. Published Application No. 2014/017265; WO 2014/093379; WO 2014/093394; WO 2014/093640, the content of which are incorporated herein by reference in their entireties.

In particular embodiments, the aggregation of the ADCs during storage or use is less than about 40% as determined by size-exclusion chromatography (SEC). In particular embodiments, the aggregation of the ADCs during storage or use is less than 35%, such as less than about 30%, such as less than about 25%, such as less than about 20%, such as less than about 15%, such as less than about 10%, such as less than about 5%, such as less than about 4%, or even less, as determined by size-exclusion chromatography (SEC).

### 4.5. Antibodies

The antibody of an ADC may be any antibody that binds, typically but not necessarily specifically, an antigen expressed on the surface of a target cell of interest. The antigen need not, but in some embodiments, is capable of internalizing an ADC bound thereto into the cell. Target cells of interest will generally include cells where induction of apoptosis via inhibition of anti-apoptotic Bcl-xL proteins is desirable, including, by way of example and not limitation, tumor cells that express or over-express Bcl-xL. Target antigens may be any protein, glycoprotein, polysaccharide, lipoprotein, etc. expressed on the target cell of interest, but will typically be proteins that are either uniquely expressed on the target cell and not on normal or healthy cells, or that are over-expressed on the target cell as compared to normal or healthy cells, such that the ADCs selectively target specific cells of interest, such as, for example, tumor cells. As will be appreciated by skilled artisans, the specific antigen, and hence antibody, selected will depend upon the identity of the desired target cell of interest. In specific embodiments, the antibody of the ADC is an antibody suitable for administration to humans.

Antibodies (Abs) and immunoglobulins (Igs) are glycoproteins having the same structural characteristics. While antibodies exhibit binding specificity to a specific target, immunoglobulins include both antibodies and other antibody-like molecules which lack target specificity. Native antibodies and immunoglobulins are usually heterotetrameric glycoproteins of about 150,000 daltons, composed of two identical light (L) chains and two identical heavy (H) chains. Each heavy chain has at one end a variable domain (VH) followed by a number of constant domains. Each light chain has a variable domain at one end (VL) and a constant domain at its other end.

References to “VH” refer to the variable region of an immunoglobulin heavy chain of an antibody, including the heavy chain of an Fv, scFv, or Fab. References to “VL” refer to the variable region of an immunoglobulin light chain, including the light chain of an Fv, scFv, dsFv or Fab.

The term “antibody” herein is used in the broadest sense and refers to an immunoglobulin molecule that specifically binds to, or is immunologically reactive with, a particular antigen, and includes polyclonal, monoclonal, genetically engineered and otherwise modified forms of antibodies, including but not limited to murine, chimeric antibodies, humanized antibodies, heteroconjugate antibodies (e.g., bispecific antibodies, diabodies, triabodies, and tetrabodies), and antigen binding fragments of antibodies, including e.g., Fab′, F(ab′)₂, Fab, Fv, rIgG, and scFv fragments. The term “scFv” refers to a single chain Fv antibody in which the variable domains of the heavy chain and the light chain from a traditional antibody have been joined to form one chain.

Antibodies may be murine, human, humanized, chimeric, or derived from other species. An antibody is a protein generated by the immune system that is capable of recognizing and binding to a specific antigen. (Janeway, C., Travers, P., Walport, M., Shlomchik (2001) Immuno Biology, 5th Ed., Garland Publishing, New York). A target antigen generally has numerous binding sites, also called epitopes, recognized by CDRs on multiple antibodies. Each antibody that specifically binds to a different epitope has a different structure. Thus, one antigen may have more than one corresponding antibody. An antibody includes a full-length immunoglobulin molecule or an immunologically active portion of a full-length immunoglobulin molecule, i.e., a molecule that contains an antigen binding site that immunospecifically binds an antigen of a target of interest or part thereof, such targets including but not limited to, cancer cell or cells that produce autoimmune antibodies associated with an autoimmune disease. The immunoglobulin disclosed herein can be of any type (e.g., IgG, IgE, IgM, IgD, and IgA), class (e.g., IgG1, IgG2, IgG3, IgG4, IgA1 and IgA2) or subclass of immunoglobulin molecule. The immunoglobulins can be derived from any species. In one aspect, however, the immunoglobulin is of human, murine, or rabbit origin.

The term “antibody fragment” refers to a portion of a full-length antibody, generally the target binding or variable region. Examples of antibody fragments include Fab, Fab′, F(ab′)₂ and Fv fragments. An “Fv” fragment is the minimum antibody fragment which contains a complete target recognition and binding site. This region consists of a dimer of one heavy and one light chain variable domain in a tight, non-covalent association (VH-VL dimer). It is in this configuration that the three CDRs of each variable domain interact to define a target binding site on the surface of the VH-VL dimer. Often, the six CDRs confer target binding specificity to the antibody. However, in some instances even a single variable domain (or half of an Fv comprising only three CDRs specific for a target) can have the ability to recognize and bind target. “Single-chain Fv” or “scFv” antibody fragments comprise the VH and VL domains of an antibody in a single polypeptide chain. Generally, the Fv polypeptide further comprises a polypeptide linker between the VH and VL domains which enables the scFv to form the desired structure for target binding. “Single domain antibodies” are composed of a single VH or VL domains which exhibit sufficient affinity to the target. In a specific embodiment, the single domain antibody is a camelized antibody (see, e.g., Riechmann, 1999, Journal of Immunological Methods 231:25-38).

The Fab fragment contains the constant domain of the light chain and the first constant domain (CH₁) of the heavy chain. Fab′ fragments differ from Fab fragments by the addition of a few residues at the carboxyl terminus of the heavy chain CH domain including one or more cysteines from the antibody hinge region. F(ab′) fragments are produced by cleavage of the disulfide bond at the hinge cysteines of the F(ab′)₂ pepsin digestion product. Additional chemical couplings of antibody fragments are known to those of ordinary skill in the art.

Both the light chain and the heavy chain variable domains have complementarity determining regions (CDRs), also known as hypervariable regions. The more highly conserved portions of variable domains are called the framework (FR). As is known in the art, the amino acid position/boundary delineating a hypervariable region of an antibody can vary, depending on the context and the various definitions known in the art. Some positions within a variable domain may be viewed as hybrid hypervariable positions in that these positions can be deemed to be within a hypervariable region under one set of criteria while being deemed to be outside a hypervariable region under a different set of criteria. One or more of these positions can also be found in extended hypervariable regions. The CDRs in each chain are held together in close proximity by the FR regions and, with the CDRs from the other chain, contribute to the formation of the target binding site of antibodies (see Kabat et al., Sequences of Proteins of Immunological Interest (National Institute of Health, Bethesda, Md. 1987). As used herein, numbering of immunoglobulin amino acid residues is done according to the immunoglobulin amino acid residue numbering system of Kabat et al., unless otherwise indicated.

In certain embodiments, the antibodies of the ADCs in the disclosure are monoclonal antibodies. The term “monoclonal antibody” (mAb) refers to an antibody that is derived from a single copy or clone, including e.g., any eukaryotic, prokaryotic, or phage clone, and not the method by which it is produced. Preferably, a monoclonal antibody of the disclosure exists in a homogeneous or substantially homogeneous population. Monoclonal antibody includes both intact molecules, as well as, antibody fragments (such as, for example, Fab and F(ab′)₂ fragments) which are capable of specifically binding to a protein. Fab and F(ab′)₂ fragments lack the Fc fragment of intact antibody, clear more rapidly from the circulation of the animal, and may have less non-specific tissue binding than an intact antibody (Wahl et al., 1983, J. Nucl. Med. 24:316). Monoclonal antibodies useful with the present disclosure can be prepared using a wide variety of techniques known in the art including the use of hybridoma, recombinant, and phage display technologies, or a combination thereof. The antibodies of the disclosure include chimeric, primatized, humanized, or human antibodies.

While in most instances antibodies are composed of only the genetically-encoded amino acids, in some embodiments non-encoded amino acids may be incorporated at specific locations to control the number of Bcl-xL inhibitors linked to the antibody, as well as their locations. Examples of non-encoded amino acids that may be incorporated into antibodies for use in controlling stoichiometry and attachment location, as well as methods for making such modified antibodies are discussed in Tian et al., 2014, Proc Nat'l Acad Sci USA 111(5):1766-1771 and Axup et al., 2012, Proc Nat'l Acad Sci USA 109(40):16101-16106, the entire contents of which are incorporated herein by reference. In certain embodiments, the non-encoded amino acids limit the number of Bcl-xL inhibitors per antibody to about 1-8 or about 2-4.

In certain embodiments, the antibody of the ADCs described herein is a chimeric antibody. The term “chimeric” antibody as used herein refers to an antibody having variable sequences derived from a non-human immunoglobulin, such as rat or mouse antibody, and human immunoglobulin constant regions, typically chosen from a human immunoglobulin template. Methods for producing chimeric antibodies are known in the art. See, e.g., Morrison, 1985, Science 229(4719):1202-7; Oi et al., 1986, BioTechniques 4:214-221; Gillies et al., 1985, J. Immunol. Methods 125:191-202; U.S. Pat. Nos. 5,807,715; 4,816,567; and 4,816,397, which are incorporated herein by reference in their entireties.

In certain embodiments, the antibody of the ADCs described herein is a humanized antibody. “Humanized” forms of non-human (e.g., murine) antibodies are chimeric immunoglobulins, immunoglobulin chains or fragments thereof (such as Fv, Fab, Fab′, F(ab′)₂ or other target-binding subdomains of antibodies) which contain minimal sequences derived from non-human immunoglobulin. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin sequence. The humanized antibody can also comprise at least a portion of an immunoglobulin constant region (Fc), typically that of a human immunoglobulin consensus sequence. Methods of antibody humanization are known in the art. See, e.g., Riechmann et al., 1988, Nature 332:323-7; U.S. Pat. Nos. 5,530,101; 5,585,089; 5,693,761; 5,693,762; and 6,180,370 to Queen et al.; EP239400; PCT publication WO 91/09967; U.S. Pat. No. 5,225,539; EP592106; EP519596; Padlan, 1991, Mol. Immunol., 28:489-498; Studnicka et al., 1994, Prot. Eng. 7:805-814; Roguska et al., 1994, Proc. Natl. Acad. Sci. 91:969-973; and U.S. Pat. No. 5,565,332, all of which are hereby incorporated by reference in their entireties.

In certain embodiments, the antibody of the ADCs described herein is a human antibody. Completely “human” antibodies can be desirable for therapeutic treatment of human patients. As used herein, “human antibodies” include antibodies having the amino acid sequence of a human immunoglobulin and include antibodies isolated from human immunoglobulin libraries or from animals transgenic for one or more human immunoglobulin and that do not express endogenous immunoglobulins. Human antibodies can be made by a variety of methods known in the art including phage display methods using antibody libraries derived from human immunoglobulin sequences. See U.S. Pat. Nos. 4,444,887 4,716,111, 6,114,598, 6,207,418, 6,235,883, 7,227,002, 8,809,151 and U.S. Published Application No. 2013/189218, the contents of which are incorporated herein by reference in their entireties. Human antibodies can also be produced using transgenic mice which are incapable of expressing functional endogenous immunoglobulins, but which can express human immunoglobulin genes. See, e.g., U.S. Pat. Nos. 5,413,923; 5,625,126; 5,633,425; 5,569,825; 5,661,016; 5,545,806; 5,814,318; 5,885,793; 5,916,771; 5,939,598; 7,723,270; 8,809,051 and U.S. Published Application No. 2013/117871, the contents of each which are incorporated by reference herein in their entireties. In addition, companies such as Medarex (Princeton, NJ), Astellas Pharma (Deerfield, IL), and Regeneron (Tarrytown, NY) can be engaged to provide human antibodies directed against a selected antigen using technology similar to that described above. Completely human antibodies that recognize a selected epitope can be generated using a technique referred to as “guided selection.” In this approach a selected non-human monoclonal antibody, e.g., a mouse antibody, is used to guide the selection of a completely human antibody recognizing the same epitope (Jespers et al., 1988, Biotechnology 12:899-903).

In certain embodiments, the antibody of the ADCs described herein is a primatized antibody. The term “primatized antibody” refers to an antibody comprising monkey variable regions and human constant regions. Methods for producing primatized antibodies are known in the art. See, e.g., U.S. Pat. Nos. 5,658,570; 5,681,722; and 5,693,780, which are incorporated herein by reference in their entireties.

In certain embodiments, the antibody of the ADCs described herein is a bispecific antibody or a dual variable domain antibody (DVD). Bispecific and DVD antibodies are monoclonal, often human or humanized, antibodies that have binding specificities for at least two different antigens. DVDs are described, for example, in U.S. Pat. No. 7,612,181, the disclosure of which is incorporated herein by reference.

In certain embodiments, the antibody of the ADCs described herein is a derivatized antibody. For example, but not by way of limitation, derivatized antibodies are typically modified by glycosylation, acetylation, pegylation, phosphorylation, amidation, derivatization by known protecting/blocking groups, proteolytic cleavage, linkage to a cellular ligand or other protein, etc. Any of numerous chemical modifications can be carried out by known techniques, including, but not limited to, specific chemical cleavage, acetylation, formylation, metabolic synthesis of tunicamycin, etc. Additionally, the derivative can contain one or more non-natural amino acids, e.g., using Ambrx technology (see, e.g., Wolfson, 2006, Chem. Biol. 13(10):1011-2).

In certain embodiments, the antibody of the ADCs described herein has a sequence that has been modified to alter at least one constant region-mediated biological effector function relative to the corresponding wild type sequence. For example, in some embodiments, the antibody can be modified to reduce at least one constant region-mediated biological effector function relative to an unmodified antibody, e.g., reduced binding to the Fc receptor (FcR). FcR binding can be reduced by mutating the immunoglobulin constant region segment of the antibody at particular regions necessary for FcR interactions (see e.g., Canfield and Morrison, 1991, J. Exp. Med. 173:1483-1491; and Lund et al., 1991, J. Immunol. 147:2657-2662).

In certain embodiments, the antibody of the ADCs described herein is modified to acquire or improve at least one constant region-mediated biological effector function relative to an unmodified antibody, e.g., to enhance FcγR interactions (See, e.g., US 2006/0134709). For example, an antibody with a constant region that binds FcγRIIA, FcγRIIB and/or FcγRIIIA with greater affinity than the corresponding wild type constant region can be produced according to the methods described herein.

In certain embodiments, the antibody of the ADCs described herein is an antibody that binds tumor cells, such as an antibody against a cell surface receptor or a tumor-associated antigen (TAA). In attempts to discover effective cellular targets for cancer diagnosis and therapy, researchers have sought to identify transmembrane or otherwise tumor-associated polypeptides that are specifically expressed on the surface of one or more particular type(s) of cancer cell as compared to one or more normal non-cancerous cell(s). Often, such tumor-associated polypeptides are more abundantly expressed on the surface of the cancer cells as compared to the surface of the non-cancerous cells. Such cell surface receptor and tumor-associated antigens are known in the art, and can prepared for use in generating antibodies using methods and information which are well known in the art.

### 4.5.1 Exemplary Cell Surface Receptors and TAAs

Examples of cell surface receptor and TAAs to which the antibody of the ADCs described herein may be targeted include, but are not limited to, the various receptors and TAAs listed below. For convenience, information relating to these antigens, all of which are known in the art, is listed below and includes names, alternative names, Genbank accession numbers and primary reference(s), following nucleic acid and protein sequence identification conventions of the National Center for Biotechnology Information (NCBI). Nucleic acid and protein sequences corresponding to the listed cell surface receptors and TAAs are available in public databases such as GenBank.
4-1BB
5AC
5T4
Alpha-fetoprotein
angiopoietin 2
ASLG659
TCL1
BMPR1B
Brevican (BCAN, BEHAB)
C242 antigen
C5
CA-125
CA-125 (imitation)
CA-IX (Carbonic anhydrase 9)
CCR4
CD140a
CD152
CD19
CD20
CD200
CD21 (C3DR) 1)
CD22 (B-cell receptor CD22-B isoform)
CD221
CD23 (gE receptor)
CD28
CD30 (TNFRSF8)
CD33
CD37
CD38 (cyclic ADP ribose hydrolase)
CD4
CD40
CD44 v6
CD51
CD52
CD56
CD70
CD72 (Lyb-2, B-cell differentiation antigen CD72)
CD74
CD79a (CD79A, CD79a, immunoglobulin-associated alpha) Genbank accession No. NP__001774.10)
CD79b (CD79B, CD79p, B29)
CD80
CEA
CEA-related antigen
ch4D5
CLDN18.2
CRIPTO (CR, CR1, CRGF, TDGF1 teratocarcinoma-derived growth factor)
CTLA-4
CXCR5
DLL4
DR5
E16 (LAT1, SLC7A5) EGFL7
EGFR
EpCAM
EphB2R (DRT, ERK, Hek5, EPHT3, Tyro5)
Episialin
ERBB3
ETBR (Endothelin type B receptor)
FCRH1 (Fc receptor-like protein 1)
FcRH2 (IFGP4, IRTA4, SPAP1, SPAP1B, SPAP1C, SH2 domain containing phosphatase anchor protein
Fibronectin extra domain-B
Folate receptor 1
Frizzled receptor
GD2
GD3 ganglioside
GEDA
GPNMB
HER1
HER2 (ErbB2)
HER2/neu
HER3
HGF
HLA-DOB
HLA-DR
Human scatter factor receptor kinase
IGF-1 receptor
IgG4
IL-13
IL20Rα (IL20Ra, ZCYTOR7)
IL-6
ILGF2
ILFR1R
integrin α
integrin α₅β₁
Integrin αᵥβ₃
IRTA2 (Immunoglobulin superfamily receptor translocation associated 2, Gene Chromosome 1q21)
Lewis-Y antigen
LY64 (RP105)
MCP-1
MDP (DPEP1)
MPF (MSLN, SMR, mesothelin, megakaryocyte potentiating factor)
MS4A1
MSG783 (RNF124, hypothetical protein FIJ20315)
MUC1
Mucin CanAg
Napi3 (NAPI-3B, NPTIIb, SLC34A2, type II sodium-dependent phosphate transporter 3b)
NCA (CEACAM6)
P2X5 (Purinergic receptor P2X ligand-gated ion channel 5)
PD-1
PDCD1
PDGF-R α
Prostate specific membrane antigen
PSCA (Prostate stem cell antigen precursor)
PSCA hlg
RANKL
RON
SDC1
Sema 5b
SLAMF7 (CS-1)
STEAP1
STEAP2 (HGNC_8639, PCANAP1, STAMP1, STEAP2, STMP, prostate cancer associated gene 1)
TAG-72
TEM1
Tenascin C
TENB2, (TMEFF2, tomoregulin, TPEF, HPP1, TR)
TGF-β
TRAIL-E2
TRAIL-R1
TRAIL-R2
TrpM4 (BR22450, FIJ20041, TRPM4, TRPM4B, transient receptor potential cation channel subfamily M, member 4)
TA CTAA16.88
TWEAK-R
TYRP1 (glycoprotein 75)
VEGF
VEGF-A
EGFR-1
VEGFR-2
Vimentin

### 4.5.2 Exemplary Antibodies

Exemplary antibodies to be used with ADCs of the disclosure include but are not limited to 3F8 (GD2), Abagovomab (CA-125 (imitation)), Adecatumumab (EpCAM), Afutuzumab (CD20), Alacizumab pegol (VEGFR2), ALD518 (IL-6), Alemtuzumab (CD52), Altumomab pentetate (CEA), Amatuximab (Mesothelin), Anatumomab mafenatox (TAG-72), Apolizumab (HLA-DR), Arcitumomab (CEA), Bavituximab (Phosphatidylserine), Bectumomab (CD22), Belimumab (BAFF), Besilesomab (CEA-related antigen), Bevacizumab (VEGF-A), Bivatuzumab mertansine (CD44 v6), Blinatumomab (CD19), Brentuximab vedotin ((CD30 (TNFRSF8)), Cantuzumab mertansine (Mucin CanAg), Cantuzumab ravtansine (MUC1), Capromab pendetide (Prostatic carcinoma cells), Carlumab (MCP-1), Catumaxomab (EpCAM, CD3), CC49 (Tag-72), cBR96-DOX ADC (Lewis-Y antigen), Cetuximab (EGFR), Citatuzumab bogatox (EpCAM), Cixutumumab (IGF-1 receptor), Clivatuzumab tetraxetan(MUC1), Conatumumab (TRAIL-E2), Dacetuzumab (CD40), Dalotuzumab (Insulin-like growth factor I receptor), Daratumumab ((CD38 (cyclic ADP ribose hydrolase)), Demcizumab (DLL4), Denosumab (RANKL), Detumomab (B-lymphoma cell), Drozitumab (DR5), Dusigitumab (ILGF2), Ecromeximab (GD3 ganglioside), Eculizumab (C5), Edrecolomab (EpCAM), Elotuzumab (SLAMF7), Elsilimomab (IL-6), Enavatuzumab (TWEAK receptor), Enoticumab (DLL4), Ensituximab (5AC), Epitumomab cituxetan (Episialin), Epratuzumab (CD22), Ertumaxomab ((HER2/neu, CD3)), Etaracizumab (Integrin v03), Farletuzumab (Folate receptor 1), FBTA05 (CD20), Ficlatuzumab (HGF), Figitumumab (IGF-1 receptor), Flanvotumab ((TYRP1 (glycoprotein 75)), Fresolimumab (TGF-β), Galiximab (CD80), Ganitumab (IGF-I), Gemtuzumab ozogamicin (CD33), Girentuximab ((Carbonic anhydrase 9 (CA-IX)), Glembatumumab vedotin (GPNMB), Ibritumomab tiuxetan (CD20), Icrucumab (VEGFR-1), Igovomab (CA-125), IMAB362 (CLDN18.2), Imgatuzumab (EGFR), Indatuximab ravtansine (SDC1), Intetumumab (CD51), Inotuzumab ozogamicin (CD22), Ipilimumab (CD152), Iratumumab ((CD30 (TNFRSF8)), Labetuzumab (CEA), Lambrolizumab (PDCD1), Lexatumumab (TRAIL-R2), Lintuzumab (CD33), Lorvotuzumab mertansine (CD56), Lucatumumab (CD40), Lumiliximab ((CD23 (IgE receptor)), Mapatumumab (TRAIL-R1), Margetuximab (ch4D5), Matuzumab (EGFR), Milatuzumab (CD74), Mitumomab (GD3 ganglioside), Mogamulizumab (CCR4), Moxetumomab pasudotox (CD22), Nacolomab tafenatox (C242 antigen), Naptumomab estafenatox (5T4), Narnatumab (RON), Natalizumab (integrin α₄), Necitumumab (EGFR), Nesvacumab (angiopoietin 2), Nimotuzumab (EGFR), Nivolumab (IgG4), Ocaratuzumab (CD20), Ofatumumab (CD20), Olaratumab (PDGF-R α), Onartuzumab (Human scatter factor receptor kinase), Ontuxizumab (TEM1), Oportuzumab monato (EpCAM), Oregovomab (CA-125), Otlertuzumab (CD37), Panitumumab (EGFR), Pankomab (Tumor specific glycosylation of MUC1), Parsatuzumab (EGFL7), Patritumab (HER3), Pemtumomab (MUC1), Pertuzumab (HER2/neu), Pidilizumab (PD-1), Pinatuzumab vedotin (CD22), Pritumumab (Vimentin), Racotumomab (N-glycolylneuraminic acid), Radretumab (Fibronectin extra domain-B), Ramucirumab (VEGFR2), Rilotumumab (HGF), Rituximab (CD20), Robatumumab (IGF-1 receptor), Samalizumab (CD200), Satumomab pendetide (TAG-72), Seribantumab (ERBB3), Sibrotuzumab (FAP), SGN-CD19A (CD19), SGN-CD33A (CD33), Siltuximab (IL-6), Solitomab (EpCAM), Sonepcizumab (Sphingosine-1-phosphate), Tabalumb (BAFF), Tacatuzumab tetraxetan (Alpha-fetoprotein), Taplitumomab paptox (CD19), Tenatumomab (Tenascin C), Teprotumumab (CD221), TGN1412 (CD28), Ticilimumab (CTLA-4), Tigatuzumab (TRAIL-R2), TNX-650 (IL-13), Tovetumab (CD140a), Trastuzumab (HER2/neu), TRBS07 (GD2), Tremelimumab (CTLA-4), Tucotuzumab celmoleukin (EpCAM), Ublituximab (MS4A1), Urelumab (4-1BB), Vandetanib (VEGF), Vantictumab (Frizzled receptor), Volociximab (integrin α₅β₁), Vorsetuzumab mafodotin (CD70), Votumumab (Tumor antigen CTAA16.88), Zalutumumab (EGFR), Zanolimumab (CD4), and Zatuximab (HER1).

In certain embodiments, the antibody of the ADC binds EGFR, EpCAM, NCAM1, or CD98. In certain embodiments, the antibody of the ADC binds EGFR, EpCAM, or NCAM1. In certain embodiments, the antibody of the ADC binds EGFR or NCAM1. In certain embodiments, the antibody is selected from the group consisting of the EpCAM antibody referred to ING-1, the NCAM-1 antibody referred to as N901, and the EGFR antibody referred to as AB033.

### 4.6. Methods of Making Antibodies

The antibody of an ADC can be prepared by recombinant expression of immunoglobulin light and heavy chain genes in a host cell. For example, to express an antibody recombinantly, a host cell is transfected with one or more recombinant expression vectors carrying DNA fragments encoding the immunoglobulin light and heavy chains of the antibody such that the light and heavy chains are expressed in the host cell and, optionally, secreted into the medium in which the host cells are cultured, from which medium the antibodies can be recovered. Standard recombinant DNA methodologies are used to obtain antibody heavy and light chain genes, incorporate these genes into recombinant expression vectors and introduce the vectors into host cells, such as those described in Molecular Cloning; A Laboratory Manual, Second Edition (Sambrook, Fritsch and Maniatis (eds), Cold Spring Harbor, N. Y., 1989), Current Protocols in Molecular Biology (Ausubel, F. M. et al., eds., Greene Publishing Associates, 1989) and in U.S. Pat. No. 4,816,397.

In one embodiment, the Fc variant antibodies are similar to their wild-type equivalents but for changes in their Fc domains. To generate nucleic acids encoding such Fc variant antibodies, a DNA fragment encoding the Fc domain or a portion of the Fc domain of the wild-type antibody (referred to as the “wild-type Fc domain”) can be synthesized and used as a template for mutagenesis to generate an antibody as described herein using routine mutagenesis techniques; alternatively, a DNA fragment encoding the antibody can be directly synthesized.

Once DNA fragments encoding wild-type Fc domains are obtained, these DNA fragments can be further manipulated by standard recombinant DNA techniques, for example, to convert the constant region genes to full-length antibody chain genes. In these manipulations, a CH-encoding DNA fragment is operatively linked to another DNA fragment encoding another protein, such as an antibody variable region or a flexible linker. The term “operatively linked,” as used in this context, is intended to mean that the two DNA fragments are joined such that the amino acid sequences encoded by the two DNA fragments remain in-frame.

To express the Fc variant antibodies, DNAs encoding partial or full-length light and heavy chains, obtained as described above, are inserted into expression vectors such that the genes are operatively linked to transcriptional and translational control sequences. In this context, the term “operatively linked” is intended to mean that an antibody gene is ligated into a vector such that transcriptional and translational control sequences within the vector serve their intended function of regulating the transcription and translation of the antibody gene. The expression vector and expression control sequences are chosen to be compatible with the expression host cell used. A variant antibody light chain gene and the antibody heavy chain gene can be inserted into separate vectors or, more typically, both genes are inserted into the same expression vector.

The antibody genes are inserted into the expression vector by standard methods (e.g., ligation of complementary restriction sites on the antibody gene fragment and vector, or blunt end ligation if no restriction sites are present). Prior to insertion of the variant Fc domain sequences, the expression vector can already carry antibody variable region sequences. Additionally or alternatively, the recombinant expression vector can encode a signal peptide that facilitates secretion of the antibody chain from a host cell. The antibody chain gene can be cloned into the vector such that the signal peptide is linked in-frame to the amino terminus of the antibody chain gene. The signal peptide can be an immunoglobulin signal peptide or a heterologous signal peptide (i.e., a signal peptide from a non-immunoglobulin protein).

In addition to the antibody chain genes, the recombinant expression vectors carry regulatory sequences that control the expression of the antibody chain genes in a host cell. The term “regulatory sequence” is intended to include promoters, enhancers and other expression control elements (e.g., polyadenylation signals) that control the transcription or translation of the antibody chain genes. Such regulatory sequences are described, for example, in Goeddel, Gene Expression Technology: Methods in Enzymology 185 (Academic Press, San Diego, CA, 1990). It will be appreciated by those skilled in the art that the design of the expression vector, including the selection of regulatory sequences may depend on such factors as the choice of the host cell to be transformed, the level of expression of protein desired, etc. Suitable regulatory sequences for mammalian host cell expression include viral elements that direct high levels of protein expression in mammalian cells, such as promoters and/or enhancers derived from cytomegalovirus (CMV) (such as the CMV promoter/enhancer), Simian Virus 40 (SV40) (such as the SV40 promoter/enhancer), adenovirus, (e.g., the adenovirus major late promoter (AdMLP)) and polyoma. For further description of viral regulatory elements, and sequences thereof, see, e.g., U.S. Pat. No. 5,168,062 by Stinski, U.S. Pat. No. 4,510,245 by Bell et al., and U.S. Pat. No. 4,968,615 by Schaffner et al.

In addition to the antibody chain genes and regulatory sequences, the recombinant expression vectors can carry additional sequences, such as sequences that regulate replication of the vector in host cells (e.g., origins of replication) and selectable marker genes. The selectable marker gene facilitates selection of host cells into which the vector has been introduced (See, e.g., U.S. Pat. Nos. 4,399,216, 4,634,665 and 5,179,017, all by Axel et al.). For example, typically the selectable marker gene confers resistance to drugs, such as G418, puromycin, blasticidin, hygromycin or methotrexate, on a host cell into which the vector has been introduced. Suitable selectable marker genes include the dihydrofolate reductase (DHFR) gene (for use in DHFR host cells with methotrexate selection/amplification) and the neo gene (for G418 selection). For expression of the light and heavy chains, the expression vector(s) encoding the heavy and light chains is transfected into a host cell by standard techniques. The various forms of the term “transfection” are intended to encompass a wide variety of techniques commonly used for the introduction of exogenous DNA into a prokaryotic or eukaryotic host cell, e.g., electroporation, lipofection, calcium-phosphate precipitation, DEAE-dextran transfection and the like.

It is possible to express the antibodies in either prokaryotic or eukaryotic host cells. In certain embodiments, expression of antibodies is performed in eukaryotic cells, e.g., mammalian host cells, for optimal secretion of a properly folded and immunologically active antibody. Exemplary mammalian host cells for expressing the recombinant antibodies include Chinese Hamster Ovary (CHO cells) (including DHFR CHO cells, described in Urlaub and Chasin, 1980, Proc. Natl. Acad. Sci. USA 77:4216-4220, used with a DHFR selectable marker, e.g., as described in Kaufman and Sharp, 1982, Mol. Biol. 159:601-621), NS0 myeloma cells, COS cells, 293 cells and SP2/0 cells. When recombinant expression vectors encoding antibody genes are introduced into mammalian host cells, the antibodies are produced by culturing the host cells for a period of time sufficient to allow for expression of the antibody in the host cells or secretion of the antibody into the culture medium in which the host cells are grown. Antibodies can be recovered from the culture medium using standard protein purification methods. Host cells can also be used to produce portions of intact antibodies, such as Fab fragments or scFv molecules.

In some embodiments, the antibody of an ADC can be a bifunctional antibody. Such antibodies, in which one heavy and one light chain are specific for one antigen and the other heavy and light chain are specific for a second antigen, can be produced by crosslinking an antibody to a second antibody by standard chemical crosslinking methods. Bifunctional antibodies can also be made by expressing a nucleic acid engineered to encode a bifunctional antibody.

In certain embodiments, dual specific antibodies, i.e., antibodies that bind one antigen and a second, unrelated antigen using the same binding site, can be produced by mutating amino acid residues in the light chain and/or heavy chain CDRs. Exemplary second antigens include a proinflammatory cytokine (such as, for example, lymphotoxin, interferon-γ, or interleukin-1). Dual specific antibodies can be produced, e.g., by mutating amino acid residues in the periphery of the antigen binding site (See, e.g., Bostrom et al., 2009, Science 323:1610-1614). Dual functional antibodies can be made by expressing a nucleic acid engineered to encode a dual specific antibody.

Antibodies can also be produced by chemical synthesis (e.g., by the methods described in Solid Phase Peptide Synthesis, 2^{nd} ed., 1984 The Pierce Chemical Co., Rockford, Ill.). Antibodies can also be generated using a cell-free platform (see, e.g., Chu et al., Biochemia No. 2, 2001 (Roche Molecular Biologicals)).

Methods for recombinant expression of Fc fusion proteins are described in Flanagan et al., Methods in Molecular Biology, vol. 378: Monoclonal Antibodies: Methods and Protocols.

Once an antibody has been produced by recombinant expression, it can be purified by any method known in the art for purification of an immunoglobulin molecule, for example, by chromatography (e.g., ion exchange, affinity, particularly by affinity for antigen after Protein A or Protein G selection, and sizing column chromatography), centrifugation, differential solubility, or by any other standard technique for the purification of proteins.

Once isolated, an antibody can, if desired, be further purified, e.g., by high performance liquid chromatography (See, e.g., Fisher, Laboratory Techniques In Biochemistry And Molecular Biology (Work and Burdon, eds., Elsevier, 1980)), or by gel filtration chromatography on a Superdex™ 75 column (Pharmacia Biotech AB, Uppsala, Sweden).

### 4.7. Antibody-Drug Conjugate Synthons

Antibody-Drug Conjugate synthons are synthetic intermediates used to form ADCs. The synthons are generally compounds according to structural formula (III):

D-L-R^{x} (III)

or salts thereof, wherein D is a Bcl-xL inhibitor as previously described, L is a linker as previously described, and R is a reactive group suitable for linking the synthon to an antibody.

In specific embodiments, the intermediate synthons are compounds according to structural formulae (IIIa), (IIIb), (IIIc) and (IIId), below, or salts thereof, where the various substituents Ar¹, Ar², Z¹, Z^{2a}, Z^{2b}, R′, R¹, R², R⁴, R^{11a}, R^{11b}, R¹² and R¹³ are as previously defined for structural formulae (IIa), (IIb), (IIc) and (IId), respectively, L is a linker as previously described and R^{x} is a functional group as described above:

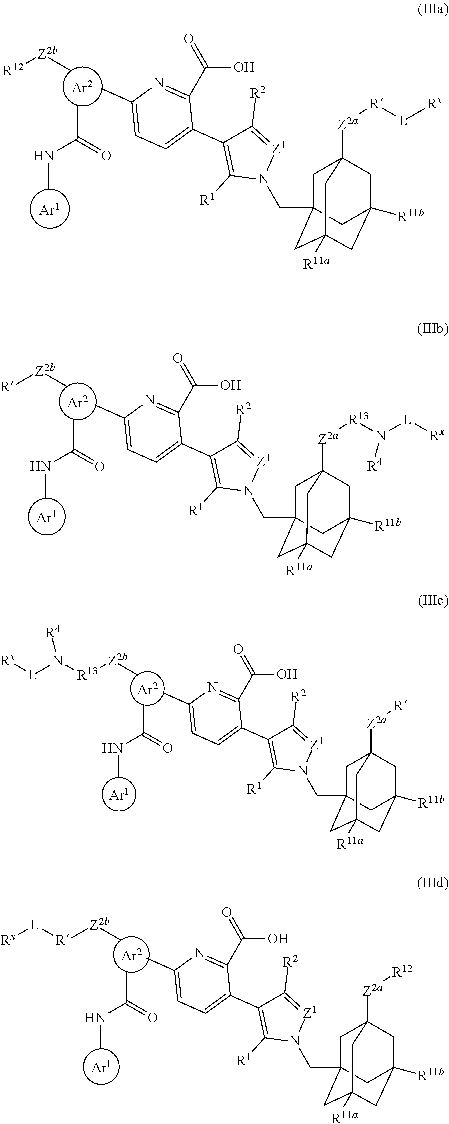

To synthesize an ADC, an intermediate synthon according to structural formula (III), or a salt thereof, is contacted with an antibody of interest under conditions in which functional group R^{x} reacts with a “complementary” functional group on the antibody, Fₓ, to form a covalent linkage.

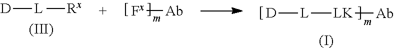

The identities of groups R^{x} and Fₓ will depend upon the chemistry used to link the synthon to the antibody. Generally, the chemistry used should not alter the integrity of the antibody, for example its ability to bind its target. Preferably, the binding properties of the conjugated antibody will closely resemble those of the unconjugated antibody. A variety of chemistries and techniques for conjugating molecules to biological molecules such as antibodies are known in the art and in particular to antibodies, are well-known. See, e.g., Amon et al., “Monoclonal Antibodies For Immunotargeting Of Drugs In Cancer Therapy,” in: Monoclonal Antibodies And Cancer Therapy, Reisfeld et al. Eds., Alan R. Liss, Inc., 1985; Hellstrom et al., “Antibodies For Drug Delivery,” in: Controlled Drug Delivery, Robinson et al., Eds., Marcel Dekker, Inc., 2nd Ed. 1987; Thorpe, “Antibody Carriers Of Cytotoxic Agents In Cancer Therapy: A Review,” in: Monoclonal Antibodies '84: Biological And Clinical Applications, Pinchera et al., Eds., 1985; “Analysis, Results, and Future Prospective of the Therapeutic Use of Radiolabeled Antibody In Cancer Therapy,” in: Monoclonal Antibodies For Cancer Detection And Therapy, Baldwin et al., Eds., Academic Press, 1985; Thorpe et al., 1982, Immunol. Rev. 62:119-58; PCT publication WO 89/12624. Any of these chemistries may be used to link the synthons to an antibody.

Typically, the synthons are linked to the side chains of amino acid residues of the antibody, including, for example, the primary amino group of accessible lysine residues or the sulfhydryl group of accessible cysteine residues. Free sulfhydryl groups may be obtained by reducing interchain disulfide bonds. In certain embodiments, LK is a linkage formed with an amino group on antibody Ab. In certain embodiments, LK is an amide, thioether, or thiourea. In certain embodiments, LK is an amide or thiourea. In certain embodiments, LK is a linkage formed with an sulfhydryl group on antibody Ab. In certain embodiments, LK is a thioether. In certain embodiments, LK is an amide, thioether, or thiourea; and m is an integer ranging from 1 to 8.

A number of functional groups R^{x} and chemistries useful for linking synthons to accessible lysine residues are known, and include by way of example and not limitation NHS-esters and isothiocyanates.

A number of functional groups R^{x} and chemistries useful for linking synthons to accessible free sulfhydryl groups of cysteine residues are known, and include by way of example and not limitation haloacetyls and maleimides.

However, conjugation chemistries are not limited to available side chain groups. Side chains such as amines may be converted to other useful groups, such as hydroxyls, by linking an appropriate small molecule to the amine. This strategy can be used to increase the number of available linking sites on the antibody by conjugating multifunctional small molecules to side chains of accessible amino acid residues of the antibody. Functional groups R^{x} suitable for covalently linking the synthons to these “converted” functional groups are then included in the synthons.

The antibody may also be engineered to include amino acid residues for conjugation. An approach for engineering antibodies to include non-genetically encoded amino acid residues useful for conjugating drugs in the context of ADCs is described in Axup et al., 2003, Proc Natl Acad Sci 109:16101-16106 and Tian et al., 2014, Proc Natl Acad Sci 111:1776-1771 as are chemistries and functional groups useful for linking synthons to the non-encoded amino acids.

Exemplary synthons that may be used to make ADCs include, but are not limited to, the following synthons:

| Exam- | Syn- | |
|---|---|---|
| ple | thon | |
| No. | Code | Synthon Structure |
| 2.1 | CZ | 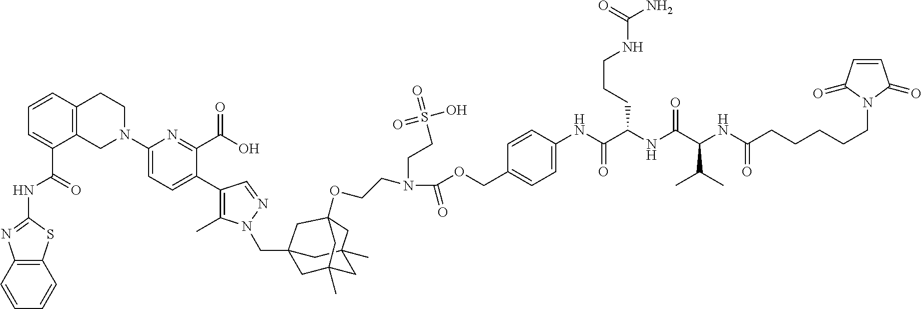 [SMILES: Cc1c(-c2ccc(N3CCc4cccc(C(=O)Nc5nc6ccccc6s5)c4C3)nc2C(=O)O)cnn1CC12CC3(C)CC(C)(C1)CC(OCCN(CCS(=O)(=O)O)C(=O)OCc1ccc(NC(=O)[C@H](CCCNC(N)=O)NC(=O)[C@@H](NC(=O)CCCCCN4C(=O)C=CC4=O)C(C)C)cc1)(C3)C2] |
| 2.2 | DH | 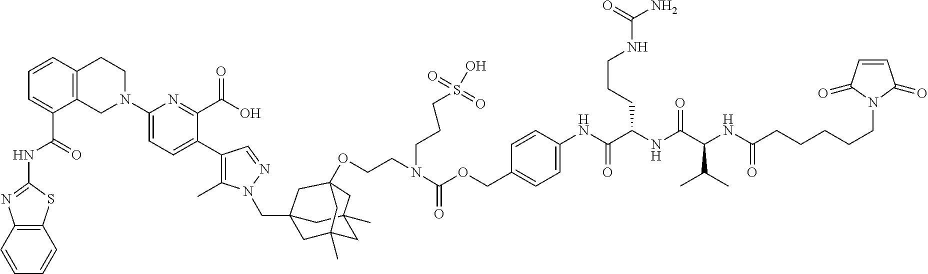 [SMILES: Cc1c(-c2ccc(N3CCc4cccc(C(=O)Nc5nc6ccccc6s5)c4C3)nc2C(=O)O)cnn1CC12CC3(C)CC(C)(C1)CC(OCCN(CCCS(=O)(=O)O)C(=O)OCc1ccc(NC(=O)[C@H](CCCNC(N)=O)NC(=O)[C@@H](NC(=O)CCCCCN4C(=O)C=CC4=O)C(C)C)cc1)(C3)C2] |
| 2.4 | EP | 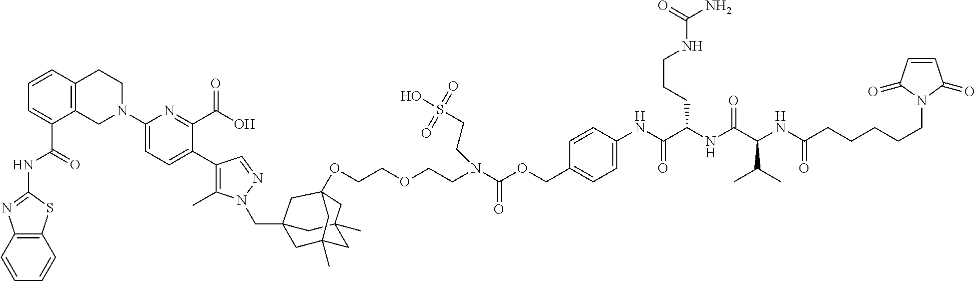 [SMILES: Cc1c(-c2ccc(N3CCc4cccc(C(=O)Nc5nc6ccccc6s5)c4C3)nc2C(=O)O)cnn1CC12CC3(C)CC(C)(C1)CC(OCCOCCN(CCS(=O)(=O)O)C(=O)OCc1ccc(NC(=O)[C@H](CCCNC(N)=O)NC(=O)[C@@H](NC(=O)CCCCCN4C(=O)C=CC4=O)C(C)C)cc1)(C3)C2] |
| 2.5 | EF | 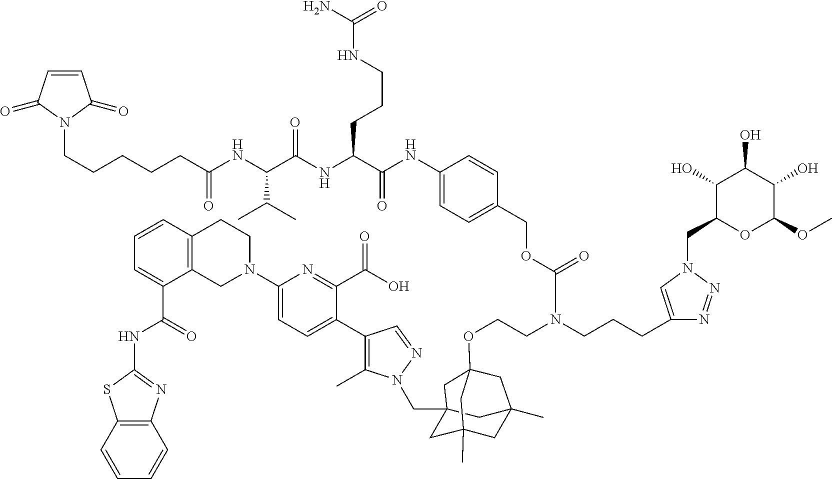 [SMILES: CO[C@H]1O[C@@H](Cn2cc(CCCN(CCOC34CC5(C)CC(C)(CC(Cn6ncc(-c7ccc(N8CCc9cccc(C(=O)Nc%10nc%11ccccc%11s%10)c9C8)nc7C(=O)O)c6C)(C5)C3)C4)C(=O)OCc3ccc(NC(=O)[C@H](CCCNC(N)=O)NC(=O)[C@@H](NC(=O)CCCCCN4C(=O)C=CC4=O)C(C)C)cc3)nn2)[C@H](O)[C@@H](O)[C@@H]1O] |
| 2.6 | EG | 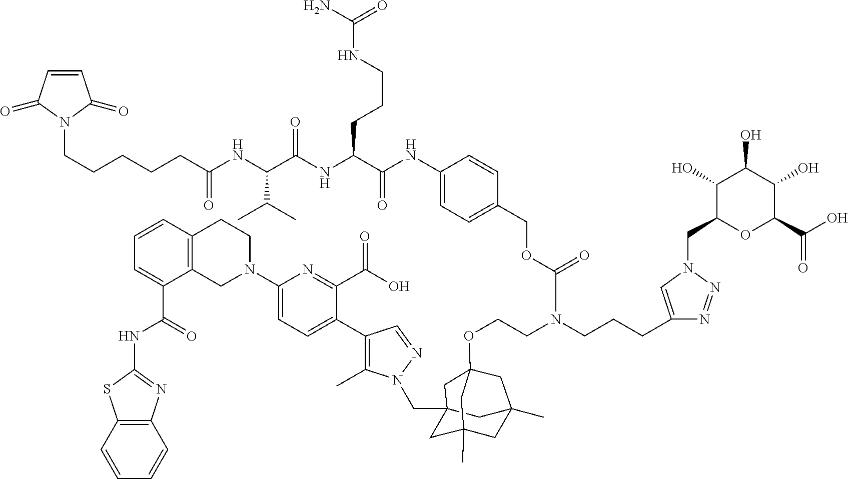 [SMILES: Cc1c(-c2ccc(N3CCc4cccc(C(=O)Nc5nc6ccccc6s5)c4C3)nc2C(=O)O)cnn1CC12CC3(C)CC(C)(C1)CC(OCCN(CCCc1cn(C[C@@H]4O[C@H](C(=O)O)[C@@H](O)[C@H](O)[C@H]4O)nn1)C(=O)OCc1ccc(NC(=O)[C@H](CCCNC(N)=O)NC(=O)[C@@H](NC(=O)CCCCCN4C(=O)C=CC4=O)C(C)C)cc1)(C3)C2] |
| 2.7 | EH | 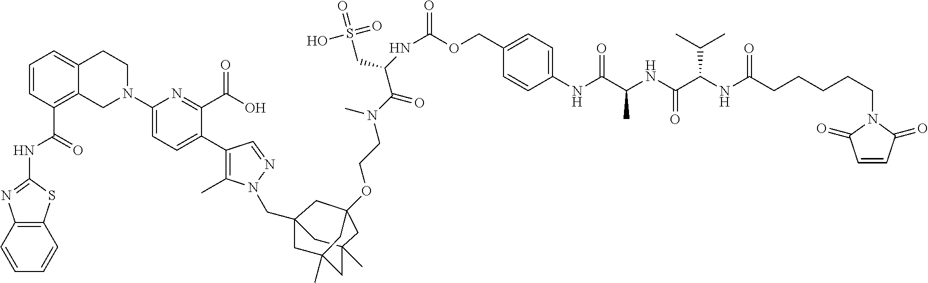 [SMILES: Cc1c(-c2ccc(N3CCc4cccc(C(=O)Nc5nc6ccccc6s5)c4C3)nc2C(=O)O)cnn1CC12CC3(C)CC(C)(C1)CC(OCCN(C)C(=O)[C@H](CS(=O)(=O)O)NC(=O)OCc1ccc(NC(=O)[C@H](C)NC(=O)[C@@H](NC(=O)CCCCCN4C(=O)C=CC4=O)C(C)C)cc1)(C3)C2] |
| 2.8 | ER | 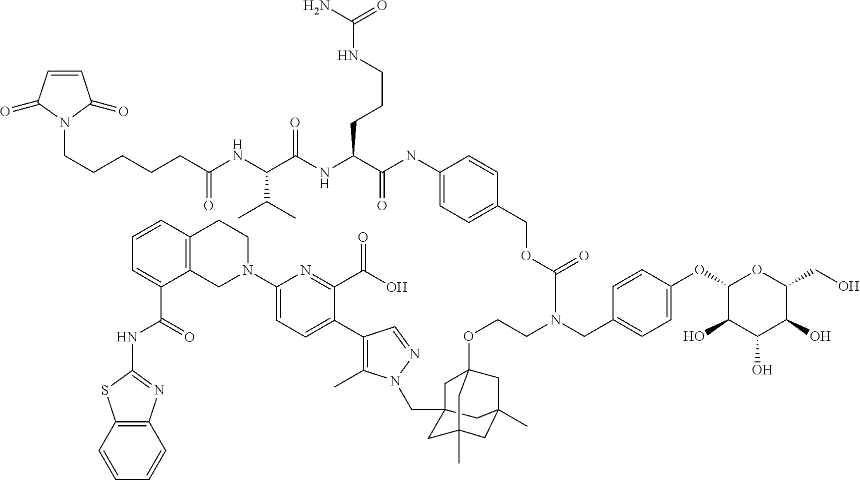 [SMILES: Cc1c(-c2ccc(N3CCc4cccc(C(=O)Nc5nc6ccccc6s5)c4C3)nc2C(=O)O)cnn1CC12CC3(C)CC(C)(C1)CC(OCCN(Cc1ccc(O[C@@H]4O[C@H](CO)[C@@H](O)[C@H](O)[C@H]4O)cc1)C(=O)OCc1ccc(NC(=O)[C@H](CCCNC(N)=O)NC(=O)[C@@H](NC(=O)CCCCCN4C(=O)C=CC4=O)C(C)C)cc1)(C3)C2] |
| 2.9 | ES | 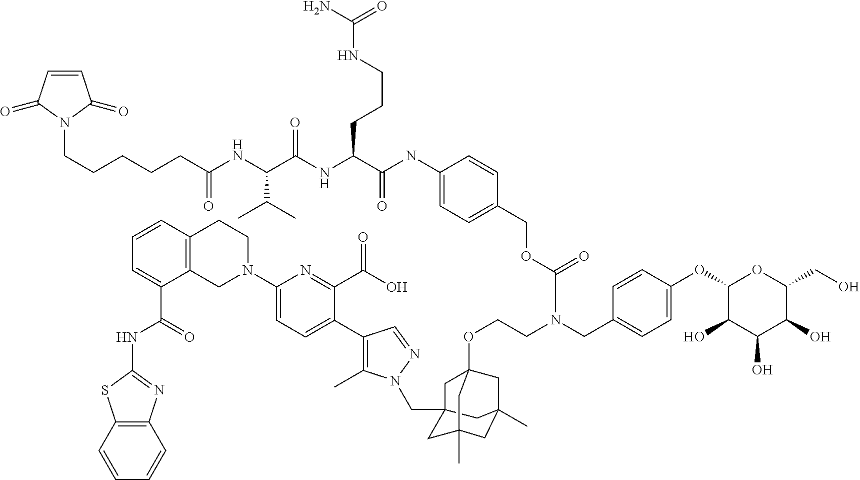 [SMILES: Cc1c(-c2ccc(N3CCc4cccc(C(=O)Nc5nc6ccccc6s5)c4C3)nc2C(=O)O)cnn1CC12CC3(C)CC(C)(C1)CC(OCCN(Cc1ccc(O[C@@H]4O[C@H](CO)[C@@H](O)[C@@H](O)[C@H]4O)cc1)C(=O)OCc1ccc(NC(=O)[C@H](CCCNC(N)=O)NC(=O)[C@@H](NC(=O)CCCCCN4C(=O)C=CC4=O)C(C)C)cc1)(C3)C2] |
| 2.10 | EQ | 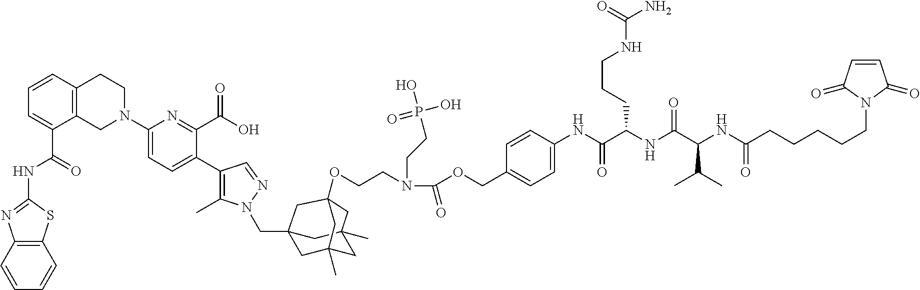 [SMILES: Cc1c(-c2ccc(N3CCc4cccc(C(=O)Nc5nc6ccccc6s5)c4C3)nc2C(=O)O)cnn1CC12CC3(C)CC(C)(C1)CC(OCCN(CCP(=O)(O)O)C(=O)OCc1ccc(NC(=O)[C@H](CCCNC(N)=O)NC(=O)[C@@H](NC(=O)CCCCCN4C(=O)C=CC4=O)C(C)C)cc1)(C3)C2] |
| 2.11 | EU | 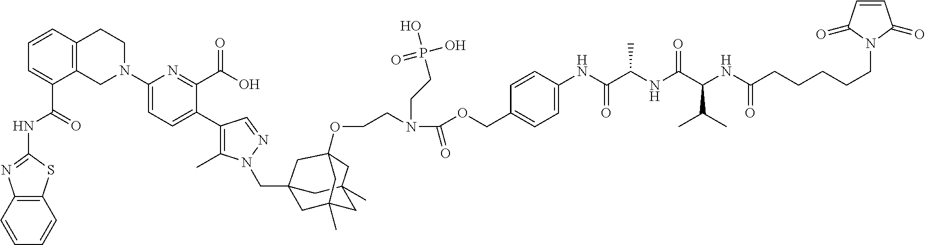 [SMILES: Cc1c(-c2ccc(N3CCc4cccc(C(=O)Nc5nc6ccccc6s5)c4C3)nc2C(=O)O)cnn1CC12CC3(C)CC(C)(C1)CC(OCCN(CCP(=O)(O)O)C(=O)OCc1ccc(NC(=O)[C@H](C)NC(=O)[C@@H](NC(=O)CCCCCN4C(=O)C=CC4=O)C(C)C)cc1)(C3)C2] |
| 2.12 | EV | 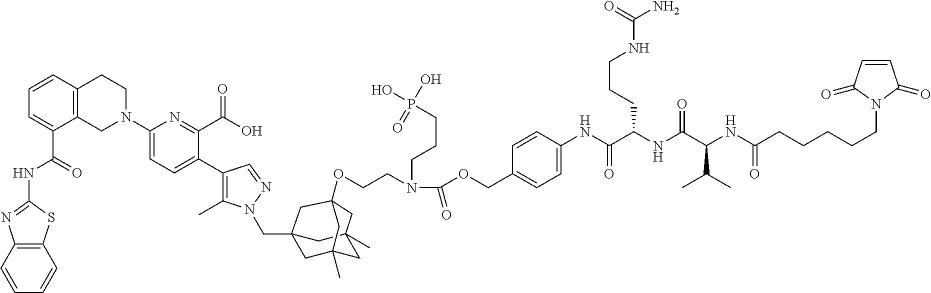 [SMILES: Cc1c(-c2ccc(N3CCc4cccc(C(=O)Nc5nc6ccccc6s5)c4C3)nc2C(=O)O)cnn1CC12CC3(C)CC(C)(C1)CC(OCCN(CCCP(=O)(O)O)C(=O)OCc1ccc(NC(=O)[C@H](CCCNC(N)=O)NC(=O)[C@@H](NC(=O)CCCCCN4C(=O)C=CC4=O)C(C)C)cc1)(C3)C2] |
| 2.13 | EW | 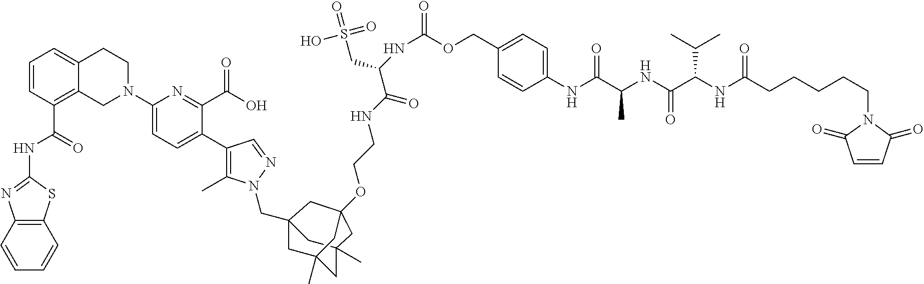 [SMILES: Cc1c(-c2ccc(N3CCc4cccc(C(=O)Nc5nc6ccccc6s5)c4C3)nc2C(=O)O)cnn1CC12CC3(C)CC(C)(C1)CC(OCCNC(=O)[C@H](CS(=O)(=O)O)NC(=O)OCc1ccc(NC(=O)[C@H](C)NC(=O)[C@@H](NC(=O)CCCCCN4C(=O)C=CC4=O)C(C)C)cc1)(C3)C2] |
| 2.14 | EX | 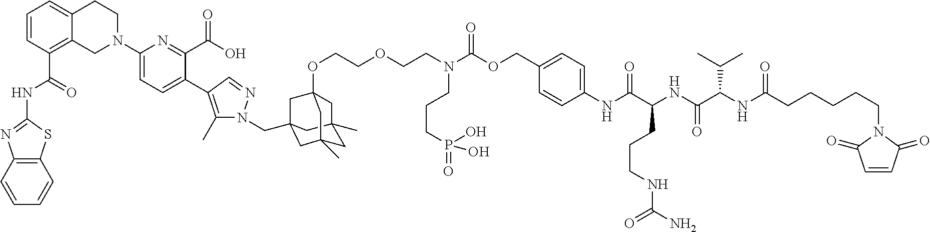 [SMILES: Cc1c(-c2ccc(N3CCc4cccc(C(=O)Nc5nc6ccccc6s5)c4C3)nc2C(=O)O)cnn1CC12CC3(C)CC(C)(C1)CC(OCCOCCN(CCCP(=O)(O)O)C(=O)OCc1ccc(NC(=O)[C@H](CCCNC(N)=O)NC(=O)[C@@H](NC(=O)CCCCCN4C(=O)C=CC4=O)C(C)C)cc1)(C3)C2] |
| 2.15 | EY | 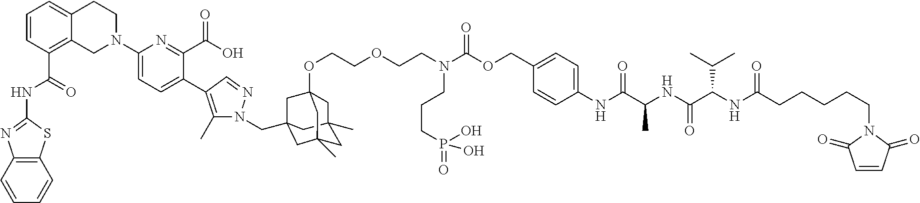 [SMILES: Cc1c(-c2ccc(N3CCc4cccc(C(=O)Nc5nc6ccccc6s5)c4C3)nc2C(=O)O)cnn1CC12CC3(C)CC(C)(C1)CC(OCCOCCN(CCCP(=O)(O)O)C(=O)OCc1ccc(NC(=O)[C@H](C)NC(=O)[C@@H](NC(=O)CCCCCN4C(=O)C=CC4=O)C(C)C)cc1)(C3)C2] |
| 2.16 | EZ | 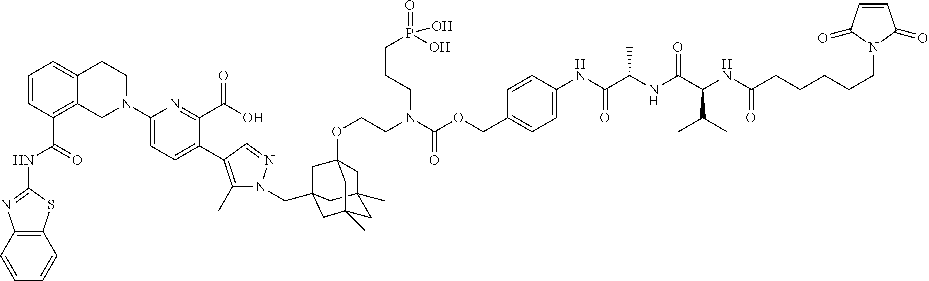 [SMILES: Cc1c(-c2ccc(N3CCc4cccc(C(=O)Nc5nc6ccccc6s5)c4C3)nc2C(=O)O)cnn1CC12CC3(C)CC(C)(C1)CC(OCCN(CCCP(=O)(O)O)C(=O)OCc1ccc(NC(=O)[C@H](C)NC(=O)[C@@H](NC(=O)CCCCCN4C(=O)C=CC4=O)C(C)C)cc1)(C3)C2] |
| 2.17 | FD | 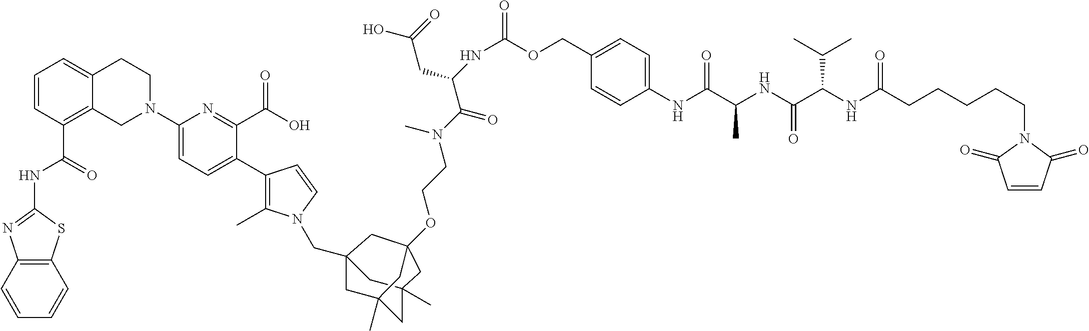 [SMILES: Cc1c(-c2ccc(N3CCc4cccc(C(=O)Nc5nc6ccccc6s5)c4C3)nc2C(=O)O)ccn1CC12CC3(C)CC(C)(C1)CC(OCCN(C)C(=O)[C@H](CC(=O)O)NC(=O)OCc1ccc(NC(=O)[C@H](C)NC(=O)[C@@H](NC(=O)CCCCCN4C(=O)C=CC4=O)C(C)C)cc1)(C3)C2] |
| 2.18 | FS | 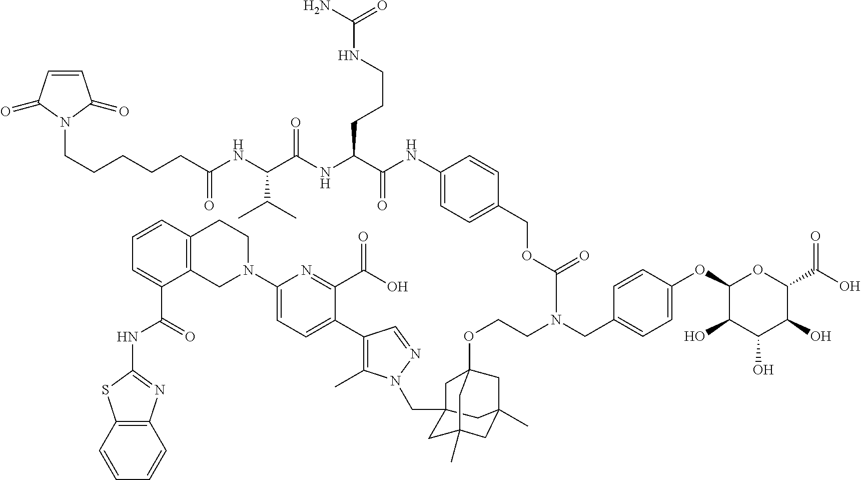 [SMILES: Cc1c(-c2ccc(N3CCc4cccc(C(=O)Nc5nc6ccccc6s5)c4C3)nc2C(=O)O)cnn1CC12CC3(C)CC(C)(C1)CC(OCCN(Cc1ccc(O[C@H]4O[C@H](C(=O)O)[C@@H](O)[C@H](O)[C@H]4O)cc1)C(=O)OCc1ccc(NC(=O)[C@H](CCCNC(N)=O)NC(=O)[C@@H](NC(=O)CCCCCN4C(=O)C=CC4=O)C(C)C)cc1)(C3)C2] |
| 2.19 | FI | 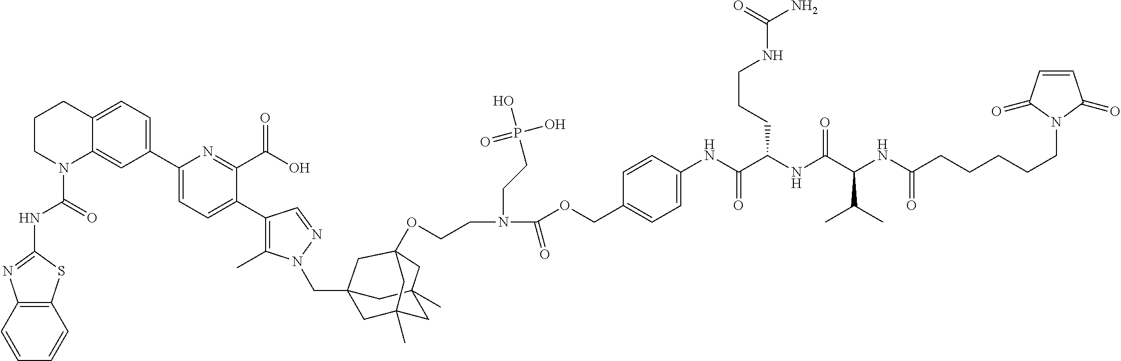 [SMILES: Cc1c(-c2ccc(-c3ccc4c(c3)N(C(=O)Nc3nc5ccccc5s3)CCC4)nc2C(=O)O)cnn1CC12CC3(C)CC(C)(C1)CC(OCCN(CCP(=O)(O)O)C(=O)OCc1ccc(NC(=O)[C@H](CCCNC(N)=O)NC(=O)[C@@H](NC(=O)CCCCCN4C(=O)C=CC4=O)C(C)C)cc1)(C3)C2] |
| 2.20 | FV | 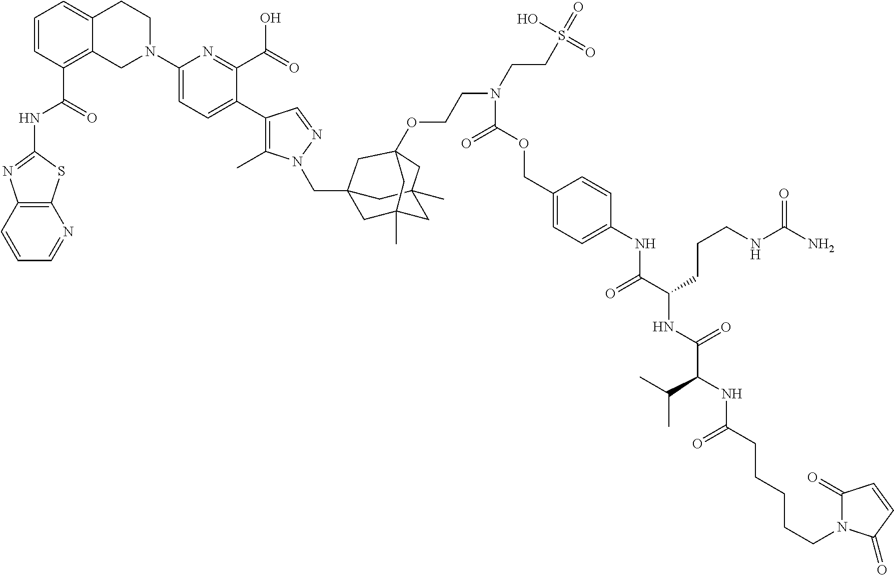 [SMILES: Cc1c(-c2ccc(N3CCc4cccc(C(=O)Nc5nc6cccnc6s5)c4C3)nc2C(=O)O)cnn1CC12CC3(C)CC(C)(C1)CC(OCCN(CCS(=O)(=O)O)C(=O)OCc1ccc(NC(=O)[C@H](CCCNC(N)=O)NC(=O)[C@@H](NC(=O)CCCCCN4C(=O)C=CC4=O)C(C)C)cc1)(C3)C2] |
| 2.21 | GC | 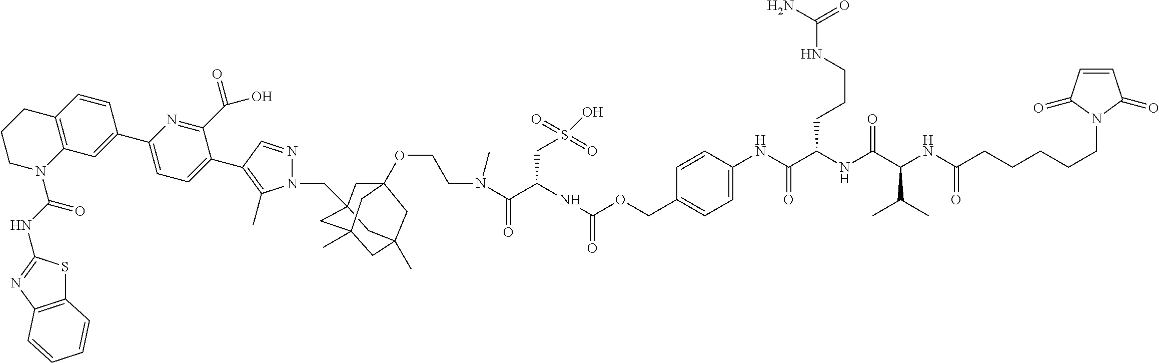 [SMILES: Cc1c(-c2ccc(-c3ccc4c(c3)N(C(=O)Nc3nc5ccccc5s3)CCC4)nc2C(=O)O)cnn1CC12CC3(C)CC(C)(C1)CC(OCCN(C)C(=O)[C@H](CS(=O)(=O)O)NC(=O)OCc1ccc(NC(=O)[C@H](CCCNC(N)=O)NC(=O)[C@@H](NC(=O)CCCCCN4C(=O)C=CC4=O)C(C)C)cc1)(C3)C2] |
| 2.22 | GB | 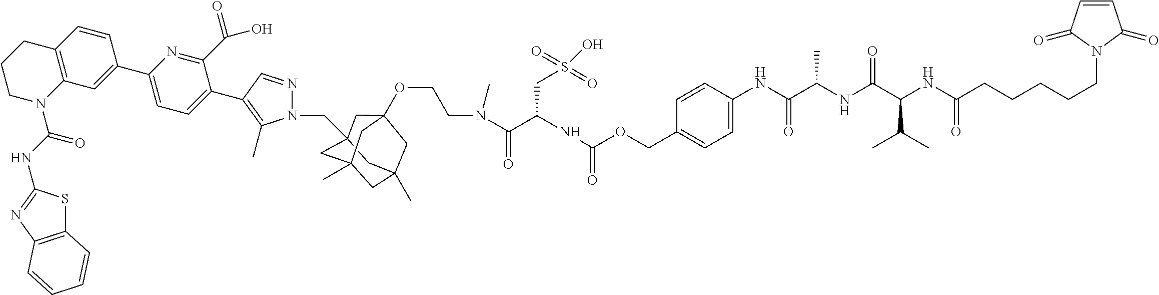 [SMILES: Cc1c(-c2ccc(-c3ccc4c(c3)N(C(=O)Nc3nc5ccccc5s3)CCC4)nc2C(=O)O)cnn1CC12CC3(C)CC(C)(C1)CC(OCCN(C)C(=O)[C@H](CS(=O)(=O)O)NC(=O)OCc1ccc(NC(=O)[C@H](C)NC(=O)[C@@H](NC(=O)CCCCCN4C(=O)C=CC4=O)C(C)C)cc1)(C3)C2] |
| 2.23 | FW | 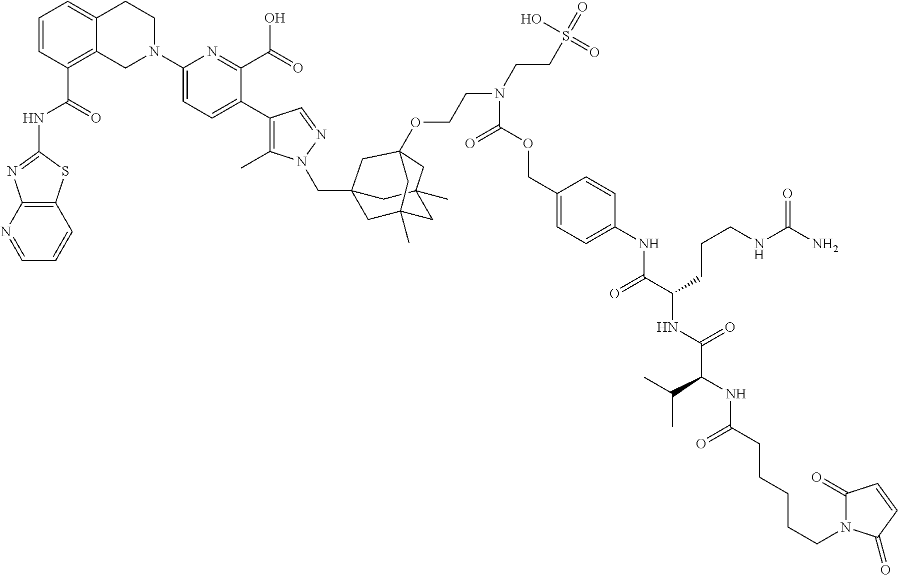 [SMILES: Cc1c(-c2ccc(N3CCc4cccc(C(=O)Nc5nc6ncccc6s5)c4C3)nc2C(=O)O)cnn1CC12CC3(C)CC(C)(C1)CC(OCCN(CCS(=O)(=O)O)C(=O)OCc1ccc(NC(=O)[C@H](CCCNC(N)=O)NC(=O)[C@@H](NC(=O)CCCCCN4C(=O)C=CC4=O)C(C)C)cc1)(C3)C2] |
| 2.24 | GD | 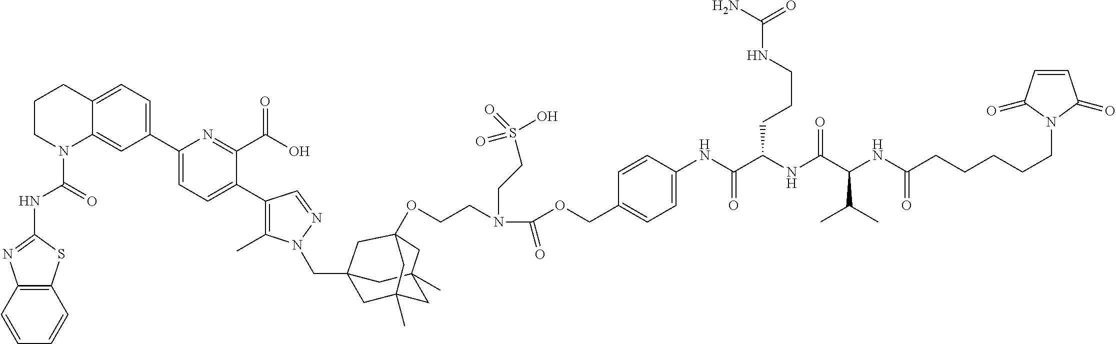 [SMILES: Cc1c(-c2ccc(-c3ccc4c(c3)N(C(=O)Nc3nc5ccccc5s3)CCC4)nc2C(=O)O)cnn1CC12CC3(C)CC(C)(C1)CC(OCCN(CCS(=O)(=O)O)C(=O)OCc1ccc(NC(=O)[C@H](CCCNC(N)=O)NC(=O)[C@@H](NC(=O)CCCCCN4C(=O)C=CC4=O)C(C)C)cc1)(C3)C2] |
| 2.25 | GK | 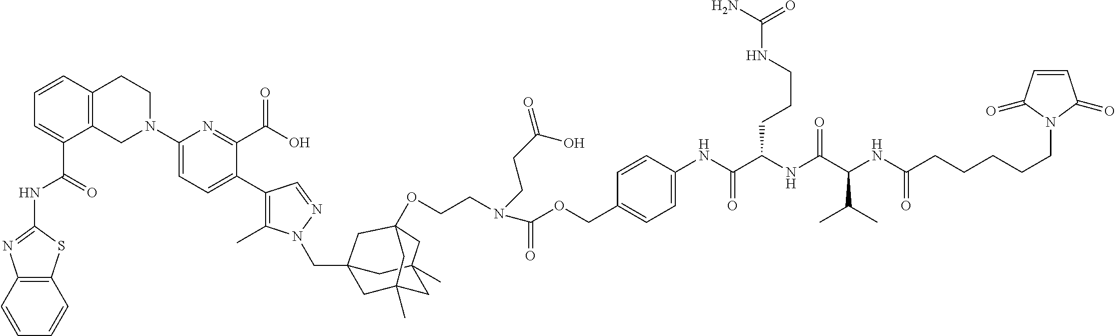 [SMILES: Cc1c(-c2ccc(N3CCc4cccc(C(=O)Nc5nc6ccccc6s5)c4C3)nc2C(=O)O)cnn1CC12CC3(C)CC(C)(C1)CC(OCCN(CCC(=O)O)C(=O)OCc1ccc(NC(=O)[C@H](CCCNC(N)=O)NC(=O)[C@@H](NC(=O)CCCCCN4C(=O)C=CC4=O)C(C)C)cc1)(C3)C2] |
| 2.26 | GJ | 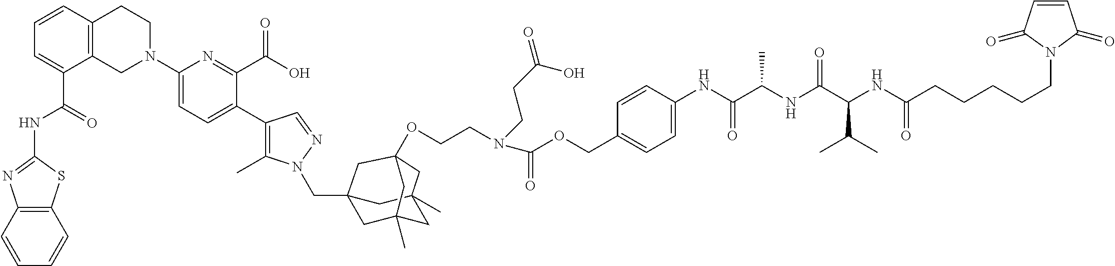 [SMILES: Cc1c(-c2ccc(N3CCc4cccc(C(=O)Nc5nc6ccccc6s5)c4C3)nc2C(=O)O)cnn1CC12CC3(C)CC(C)(C1)CC(OCCN(CCC(=O)O)C(=O)OCc1ccc(NC(=O)[C@H](C)NC(=O)[C@@H](NC(=O)CCCCCN4C(=O)C=CC4=O)C(C)C)cc1)(C3)C2] |
| 2.27 | GW | 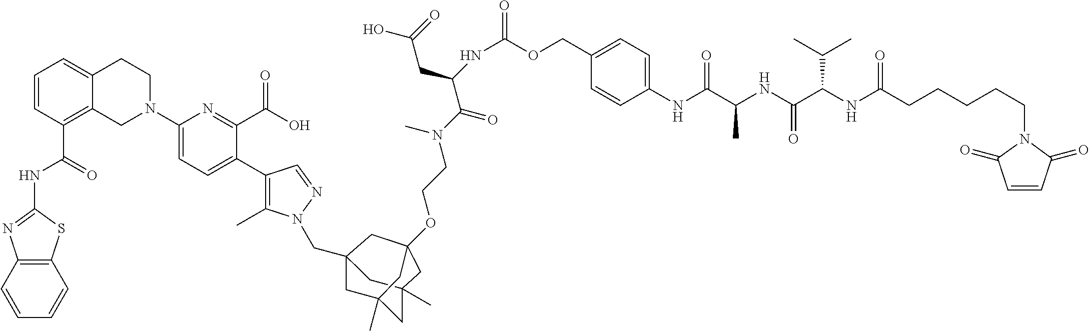 [SMILES: Cc1c(-c2ccc(N3CCc4cccc(C(=O)Nc5nc6ccccc6s5)c4C3)nc2C(=O)O)cnn1CC12CC3(C)CC(C)(C1)CC(OCCN(C)C(=O)[C@@H](CC(=O)O)NC(=O)OCc1ccc(NC(=O)[C@H](C)NC(=O)[C@@H](NC(=O)CCCCCN4C(=O)C=CC4=O)C(C)C)cc1)(C3)C2] |
| 2.28 | HF | 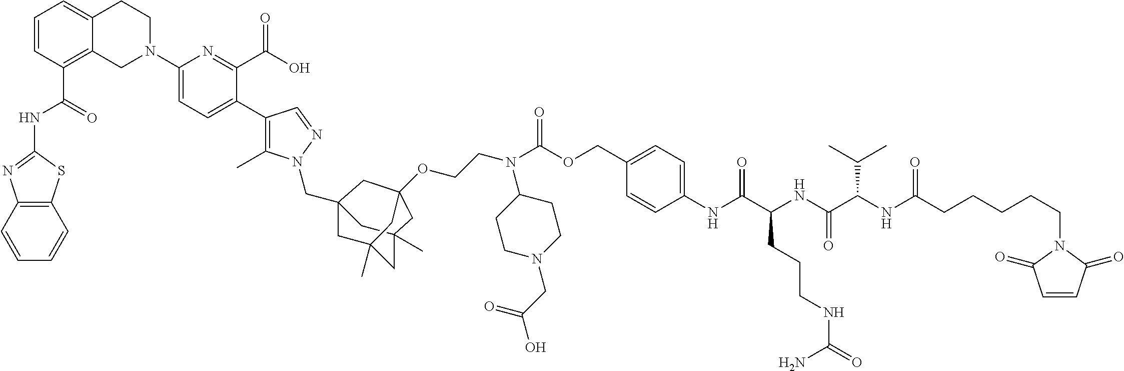 [SMILES: Cc1c(-c2ccc(N3CCc4cccc(C(=O)Nc5nc6ccccc6s5)c4C3)nc2C(=O)O)cnn1CC12CC3(C)CC(C)(C1)CC(OCCN(C(=O)OCc1ccc(NC(=O)[C@H](CCCNC(N)=O)NC(=O)[C@@H](NC(=O)CCCCCN4C(=O)C=CC4=O)C(C)C)cc1)C1CCN(CC(=O)O)CC1)(C3)C2] |
| 2.29 | HG | 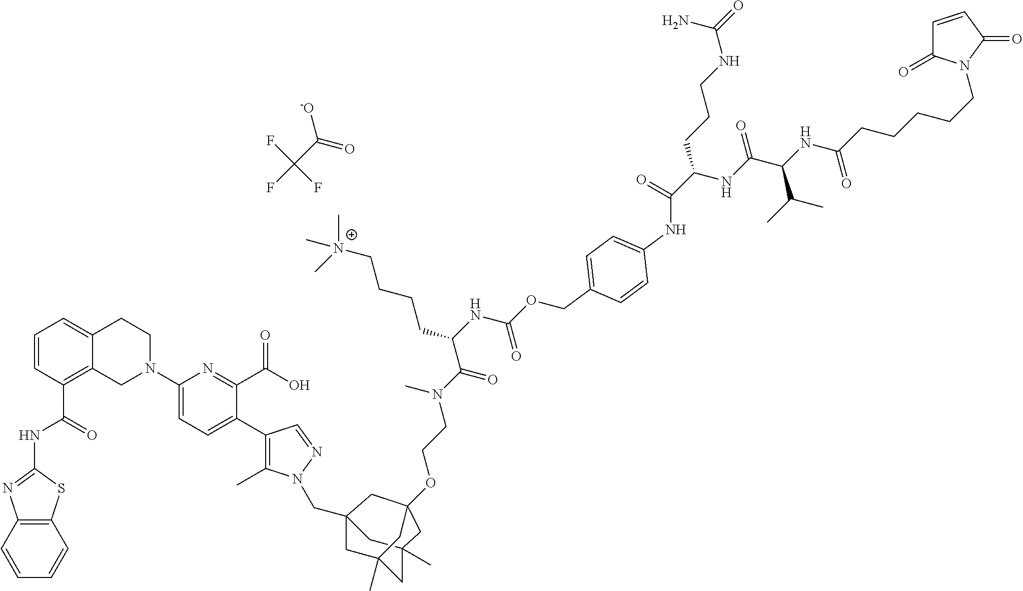 |
| 2.30 | HP | 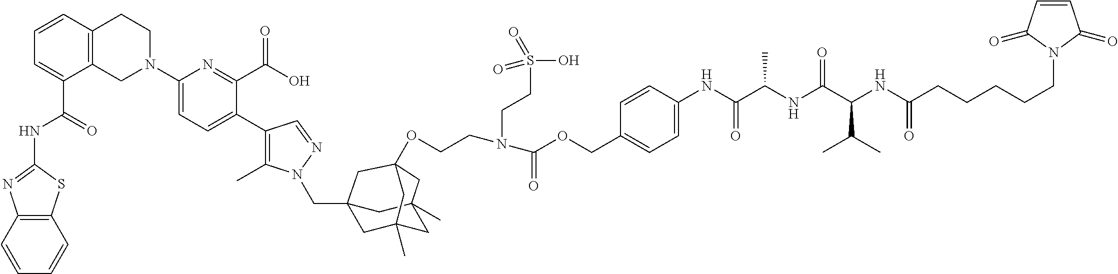 [SMILES: Cc1c(-c2ccc(N3CCc4cccc(C(=O)Nc5nc6ccccc6s5)c4C3)nc2C(=O)O)cnn1CC12CC3(C)CC(C)(C1)CC(OCCN(CCS(=O)(=O)O)C(=O)OCc1ccc(NC(=O)[C@H](C)NC(=O)[C@@H](NC(=O)CCCCCN4C(=O)C=CC4=O)C(C)C)cc1)(C3)C2] |
| 2.31 | HR | 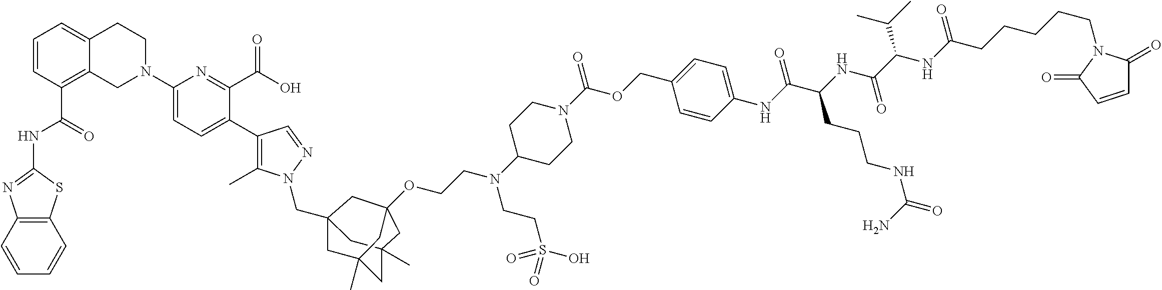 [SMILES: Cc1c(-c2ccc(N3CCc4cccc(C(=O)Nc5nc6ccccc6s5)c4C3)nc2C(=O)O)cnn1CC12CC3(C)CC(C)(C1)CC(OCCN(CCS(=O)(=O)O)C1CCN(C(=O)OCc4ccc(NC(=O)[C@H](CCCNC(N)=O)NC(=O)[C@@H](NC(=O)CCCCCN5C(=O)C=CC5=O)C(C)C)cc4)CC1)(C3)C2] |
| 2.32 | HU | 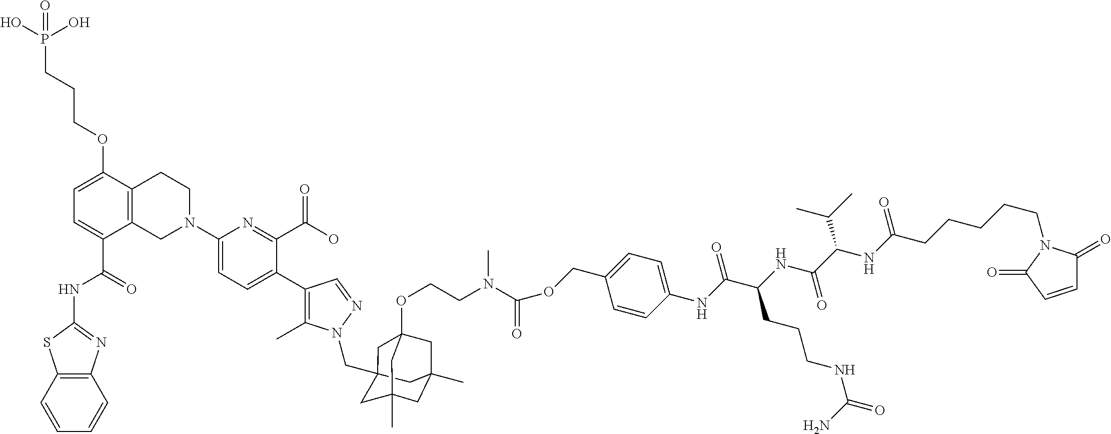 [SMILES: Cc1c(-c2ccc(N3CCc4c(OCCCP(=O)(O)O)ccc(C(=O)Nc5nc6ccccc6s5)c4C3)nc2C(=O)O)cnn1CC12CC3(C)CC(C)(C1)CC(OCCN(C)C(=O)OCc1ccc(NC(=O)[C@H](CCCNC(N)=O)NC(=O)[C@@H](NC(=O)CCCCCN4C(=O)C=CC4=O)C(C)C)cc1)(C3)C2] |
| 2.33 | HT | 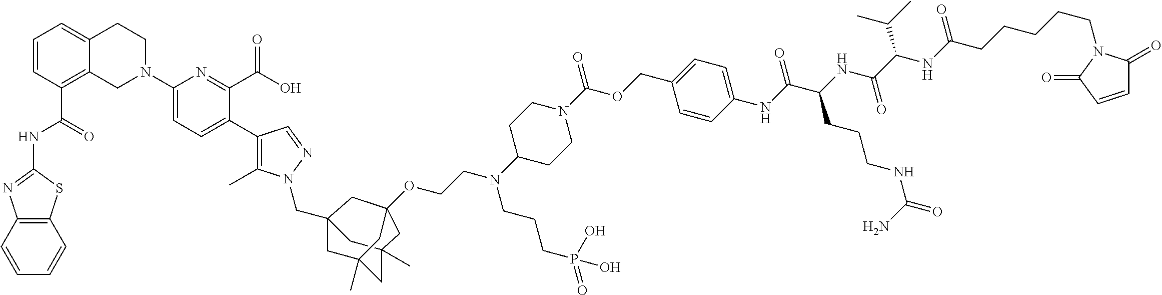 [SMILES: Cc1c(-c2ccc(N3CCc4cccc(C(=O)Nc5nc6ccccc6s5)c4C3)nc2C(=O)O)cnn1CC12CC3(C)CC(C)(C1)CC(OCCN(CCCP(=O)(O)O)C1CCN(C(=O)OCc4ccc(NC(=O)[C@H](CCCNC(N)=O)NC(=O)[C@@H](NC(=O)CCCCCN5C(=O)C=CC5=O)C(C)C)cc4)CC1)(C3)C2] |
| 2.34 | HV | 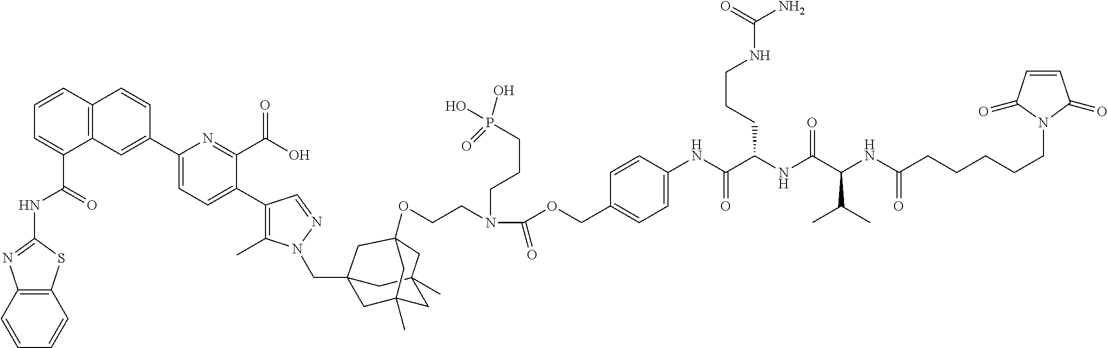 [SMILES: Cc1c(-c2ccc(-c3ccc4cccc(C(=O)Nc5nc6ccccc6s5)c4c3)nc2C(=O)O)cnn1CC12CC3(C)CC(C)(C1)CC(OCCN(CCCP(=O)(O)O)C(=O)OCc1ccc(NC(=O)[C@H](CCCNC(N)=O)NC(=O)[C@@H](NC(=O)CCCCCN4C(=O)C=CC4=O)C(C)C)cc1)(C3)C2] |
| 2.35 | HZ | 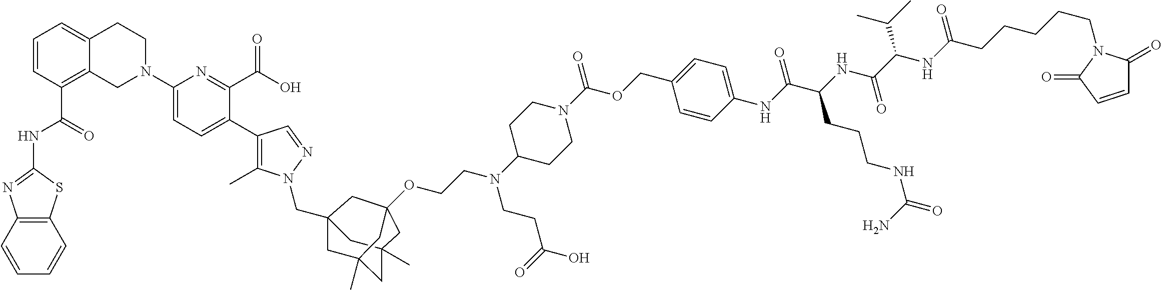 [SMILES: Cc1c(-c2ccc(N3CCc4cccc(C(=O)Nc5nc6ccccc6s5)c4C3)nc2C(=O)O)cnn1CC12CC3(C)CC(C)(C1)CC(OCCN(CCC(=O)O)C1CCN(C(=O)OCc4ccc(NC(=O)[C@H](CCCNC(N)=O)NC(=O)[C@@H](NC(=O)CCCCCN5C(=O)C=CC5=O)C(C)C)cc4)CC1)(C3)C2] |
| 2.36 | IA | 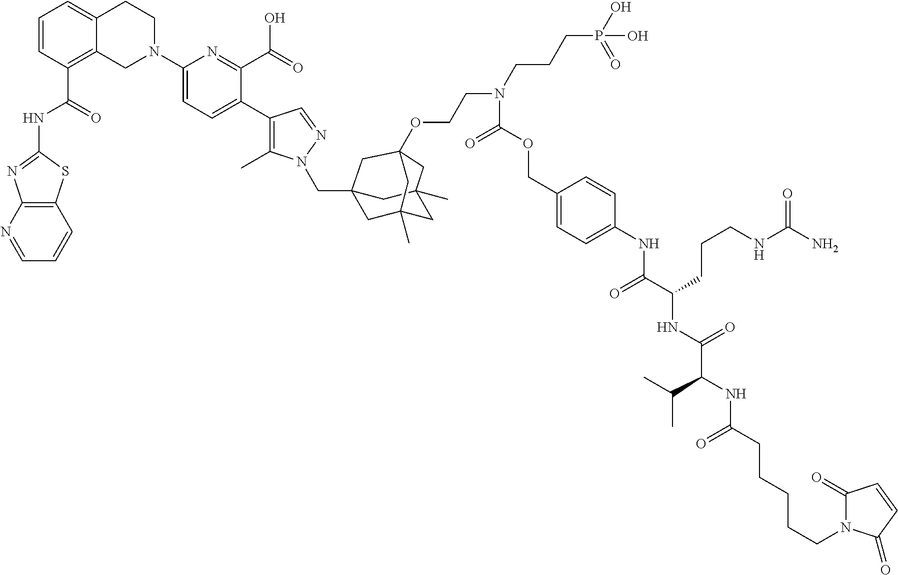 [SMILES: Cc1c(-c2ccc(N3CCc4cccc(C(=O)Nc5nc6ncccc6s5)c4C3)nc2C(=O)O)cnn1CC12CC3(C)CC(C)(C1)CC(OCCN(CCCP(=O)(O)O)C(=O)OCc1ccc(NC(=O)[C@H](CCCNC(N)=O)NC(=O)[C@@H](NC(=O)CCCCCN4C(=O)C=CC4=O)C(C)C)cc1)(C3)C2] |
| 2.37 | IF | 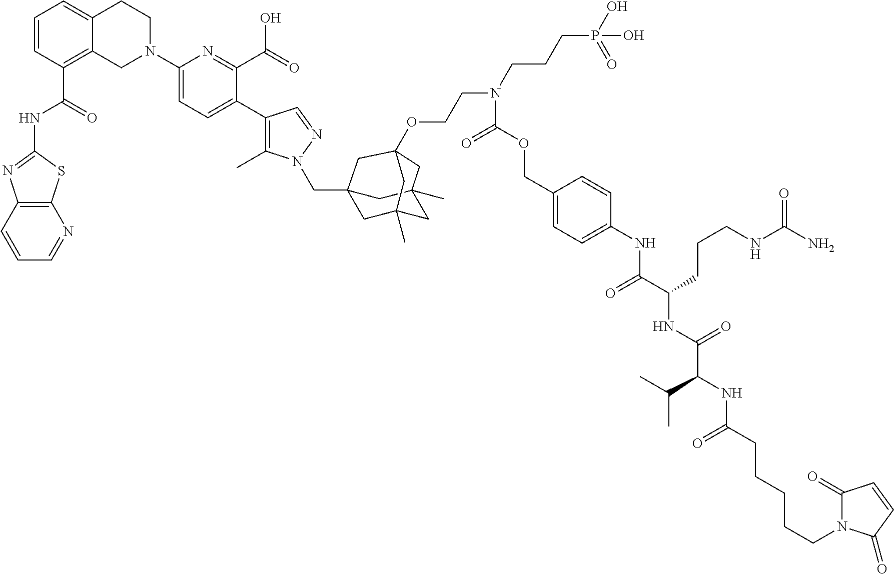 [SMILES: Cc1c(-c2ccc(N3CCc4cccc(C(=O)Nc5nc6cccnc6s5)c4C3)nc2C(=O)O)cnn1CC12CC3(C)CC(C)(C1)CC(OCCN(CCCP(=O)(O)O)C(=O)OCc1ccc(NC(=O)[C@H](CCCNC(N)=O)NC(=O)[C@@H](NC(=O)CCCCCN4C(=O)C=CC4=O)C(C)C)cc1)(C3)C2] |
| 2.38 | IG | 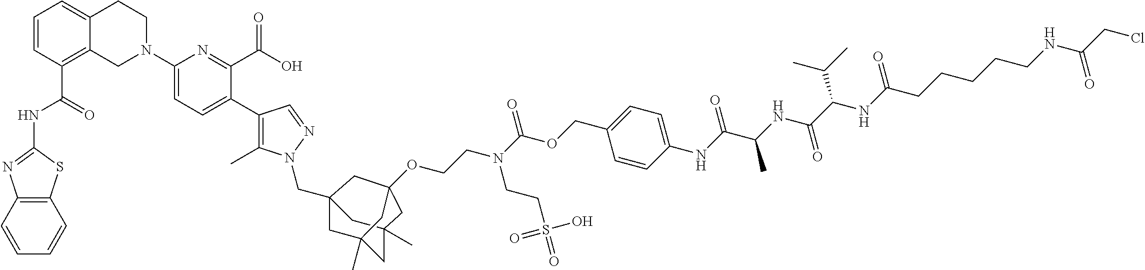 [SMILES: Cc1c(-c2ccc(N3CCc4cccc(C(=O)Nc5nc6ccccc6s5)c4C3)nc2C(=O)O)cnn1CC12CC3(C)CC(C)(C1)CC(OCCN(CCS(=O)(=O)O)C(=O)OCc1ccc(NC(=O)[C@H](C)NC(=O)[C@@H](NC(=O)CCCCCNC(=O)CCl)C(C)C)cc1)(C3)C2] |
| 2.39 | IH | 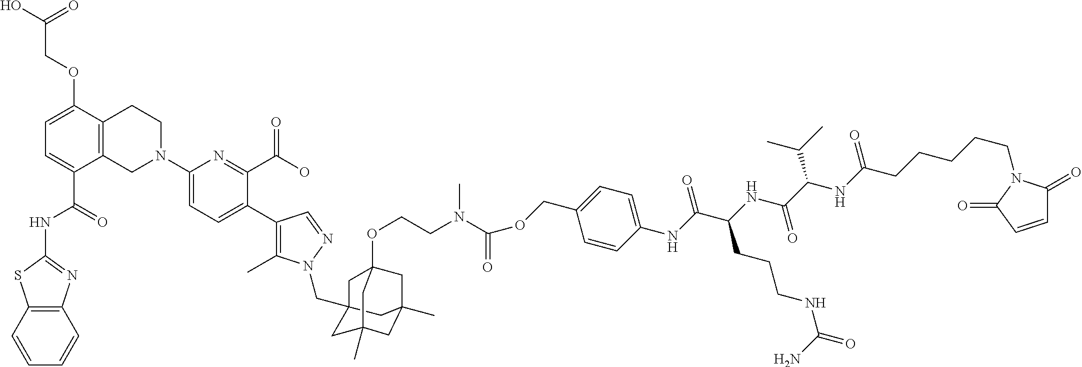 [SMILES: Cc1c(-c2ccc(N3CCc4c(OCC(=O)O)ccc(C(=O)Nc5nc6ccccc6s5)c4C3)nc2C(=O)O)cnn1CC12CC3(C)CC(C)(C1)CC(OCCN(C)C(=O)OCc1ccc(NC(=O)[C@H](CCCNC(N)=O)NC(=O)[C@@H](NC(=O)CCCCCN4C(=O)C=CC4=O)C(C)C)cc1)(C3)C2] |
| 2.40 | IJ | 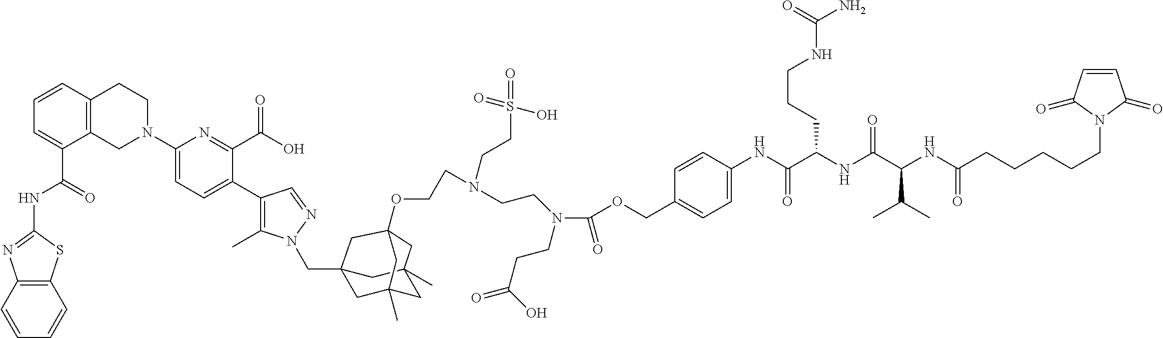 [SMILES: Cc1c(-c2ccc(N3CCc4cccc(C(=O)Nc5nc6ccccc6s5)c4C3)nc2C(=O)O)cnn1CC12CC3(C)CC(C)(C1)CC(OCCN(CCN(CCC(=O)O)C(=O)OCc1ccc(NC(=O)[C@H](CCCNC(N)=O)NC(=O)[C@@H](NC(=O)CCCCCN4C(=O)C=CC4=O)C(C)C)cc1)CCS(=O)(=O)O)(C3)C2] |
| 2.41 | IK | 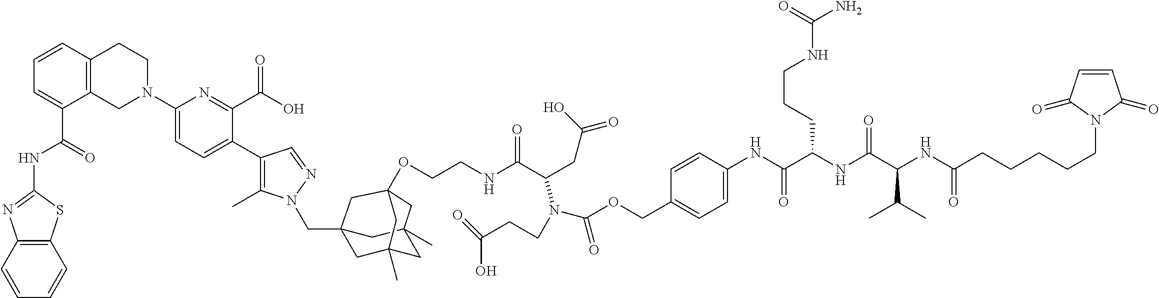 |
| 2.42 | IL | 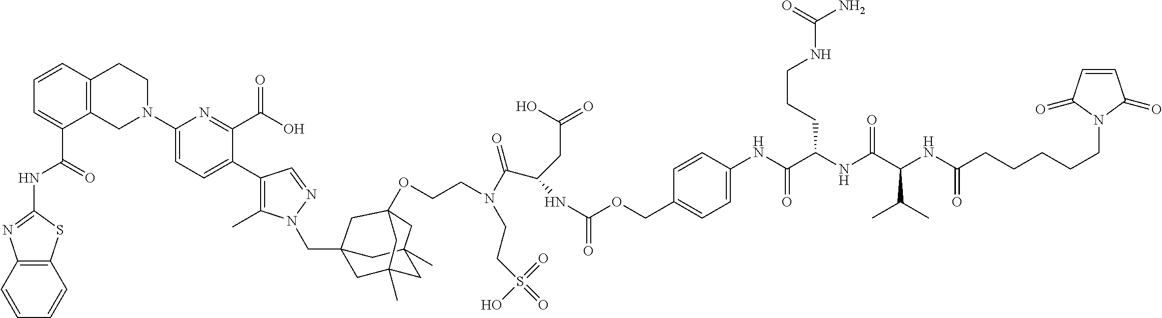 [SMILES: Cc1c(-c2ccc(N3CCc4cccc(C(=O)Nc5nc6ccccc6s5)c4C3)nc2C(=O)O)cnn1CC12CC3(C)CC(C)(C1)CC(OCCN(CCS(=O)(=O)O)C(=O)[C@H](CC(=O)O)NC(=O)OCc1ccc(NC(=O)[C@H](CCCNC(N)=O)NC(=O)[C@@H](NC(=O)CCCCCN4C(=O)C=CC4=O)C(C)C)cc1)(C3)C2] |
| 2.43 | IM | 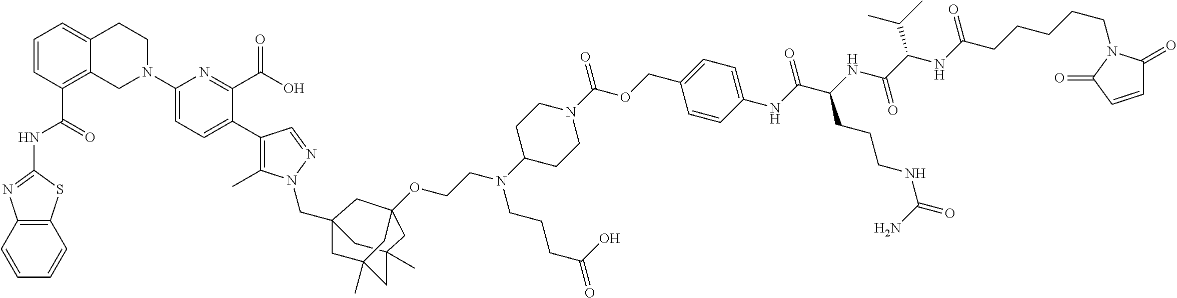 [SMILES: Cc1c(-c2ccc(N3CCc4cccc(C(=O)Nc5nc6ccccc6s5)c4C3)nc2C(=O)O)cnn1CC12CC3(C)CC(C)(C1)CC(OCCN(CCCC(=O)O)C1CCN(C(=O)OCc4ccc(NC(=O)[C@H](CCCNC(N)=O)NC(=O)[C@@H](NC(=O)CCCCCN5C(=O)C=CC5=O)C(C)C)cc4)CC1)(C3)C2] |
| 2.44 | IO | 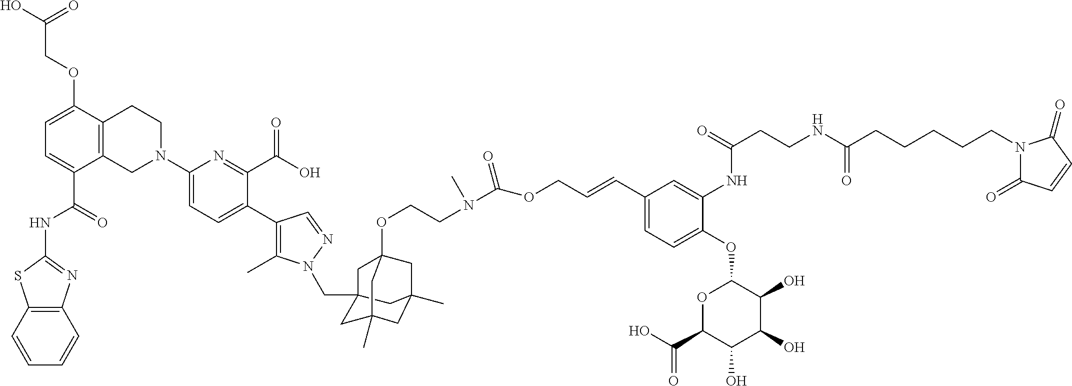 [SMILES: Cc1c(-c2ccc(N3CCc4c(OCC(=O)O)ccc(C(=O)Nc5nc6ccccc6s5)c4C3)nc2C(=O)O)cnn1CC12CC3(C)CC(C)(C1)CC(OCCN(C)C(=O)OC/C=C/c1ccc(O[C@H]4O[C@H](C(=O)O)[C@@H](O)[C@H](O)[C@@H]4O)c(NC(=O)CCNC(=O)CCCCCN4C(=O)C=CC4=O)c1)(C3)C2] |
| 2.45 | IP | 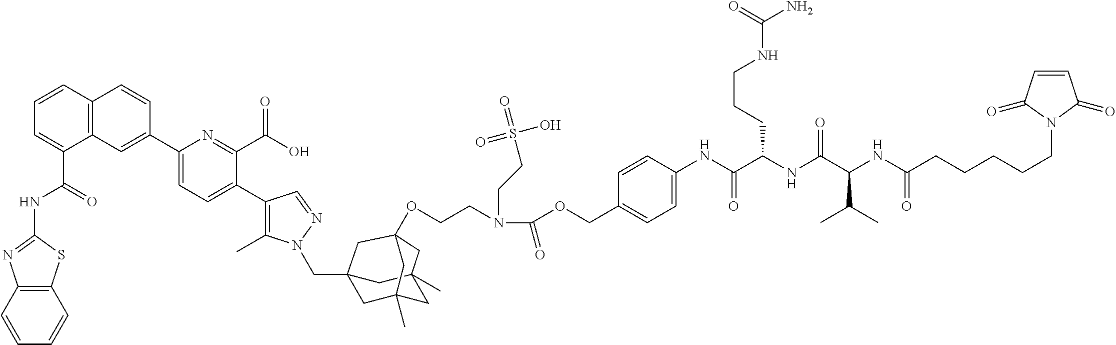 [SMILES: Cc1c(-c2ccc(-c3ccc4cccc(C(=O)Nc5nc6ccccc6s5)c4c3)nc2C(=O)O)cnn1CC12CC3(C)CC(C)(C1)CC(OCCN(CCS(=O)(=O)O)C(=O)OCc1ccc(NC(=O)[C@H](CCCNC(N)=O)NC(=O)[C@@H](NC(=O)CCCCCN4C(=O)C=CC4=O)C(C)C)cc1)(C3)C2] |
| 2.46 | IS | 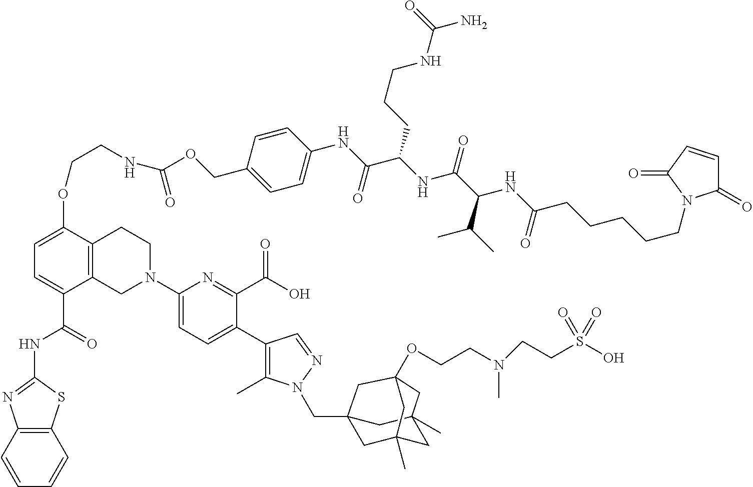 [SMILES: Cc1c(-c2ccc(N3CCc4c(OCCNC(=O)OCc5ccc(NC(=O)[C@H](CCCNC(N)=O)NC(=O)[C@@H](NC(=O)CCCCCN6C(=O)C=CC6=O)C(C)C)cc5)ccc(C(=O)Nc5nc6ccccc6s5)c4C3)nc2C(=O)O)cnn1CC12CC3(C)CC(C)(C1)CC(OCCN(C)CCS(=O)(=O)O)(C3)C2] |
| 2.47 | IU | 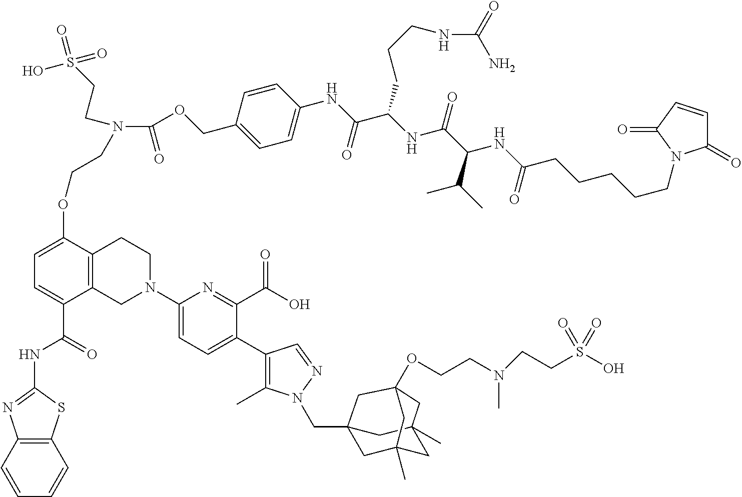 [SMILES: Cc1c(-c2ccc(N3CCc4c(OCCN(CCS(=O)(=O)O)C(=O)OCc5ccc(NC(=O)[C@H](CCCNC(N)=O)NC(=O)[C@@H](NC(=O)CCCCCN6C(=O)C=CC6=O)C(C)C)cc5)ccc(C(=O)Nc5nc6ccccc6s5)c4C3)nc2C(=O)O)cnn1CC12CC3(C)CC(C)(C1)CC(OCCN(C)CCS(=O)(=O)O)(C3)C2] |
| 2.48 | IV | 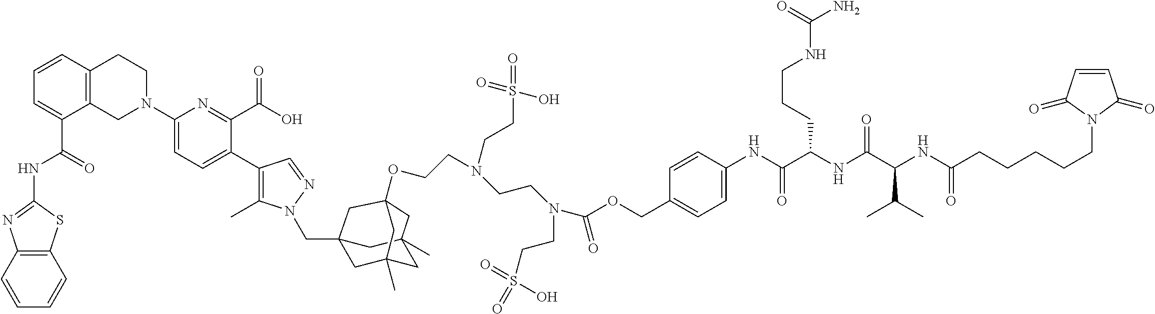 [SMILES: Cc1c(-c2ccc(N3CCc4cccc(C(=O)Nc5nc6ccccc6s5)c4C3)nc2C(=O)O)cnn1CC12CC3(C)CC(C)(C1)CC(OCCN(CCN(CCS(=O)(=O)O)C(=O)OCc1ccc(NC(=O)[C@H](CCCNC(N)=O)NC(=O)[C@@H](NC(=O)CCCCCN4C(=O)C=CC4=O)C(C)C)cc1)CCS(=O)(=O)O)(C3)C2] |
| 2.49 | IZ | 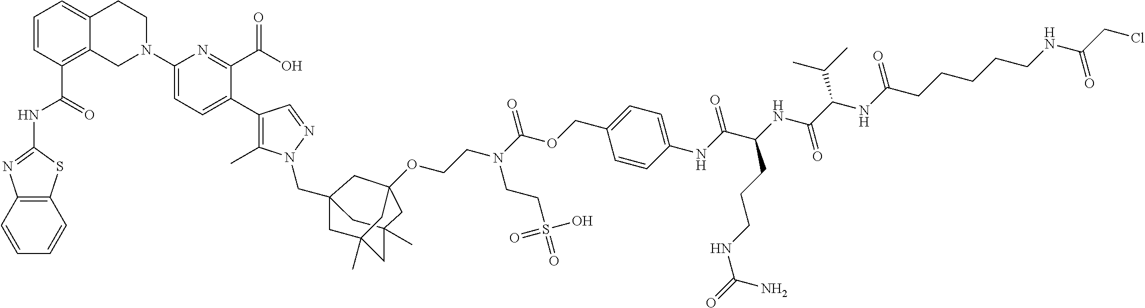 [SMILES: Cc1c(-c2ccc(N3CCc4cccc(C(=O)Nc5nc6ccccc6s5)c4C3)nc2C(=O)O)cnn1CC12CC3(C)CC(C)(C1)CC(OCCN(CCS(=O)(=O)O)C(=O)OCc1ccc(NC(=O)[C@H](CCCNC(N)=O)NC(=O)[C@@H](NC(=O)CCCCCNC(=O)CCl)C(C)C)cc1)(C3)C2] |
| 2.50 | JD | 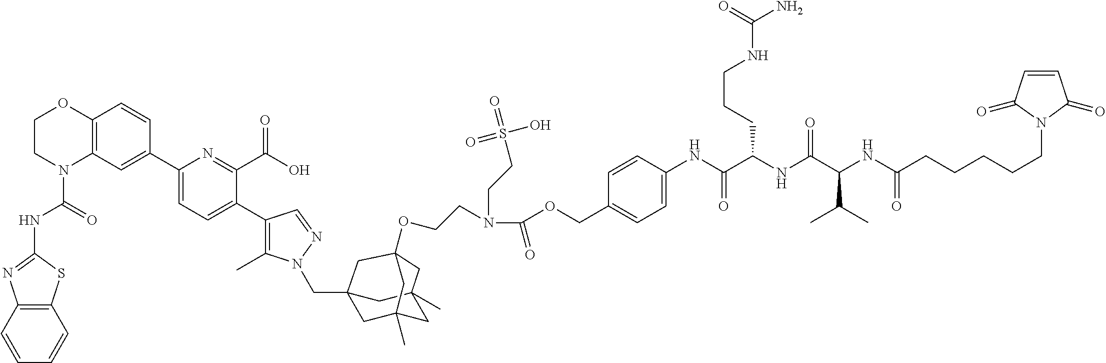 [SMILES: Cc1c(-c2ccc(-c3ccc4c(c3)N(C(=O)Nc3nc5ccccc5s3)CCO4)nc2C(=O)O)cnn1CC12CC3(C)CC(C)(C1)CC(OCCN(CCS(=O)(=O)O)C(=O)OCc1ccc(NC(=O)[C@H](CCCNC(N)=O)NC(=O)[C@@H](NC(=O)CCCCCN4C(=O)C=CC4=O)C(C)C)cc1)(C3)C2] |
| 2.51 | JF | 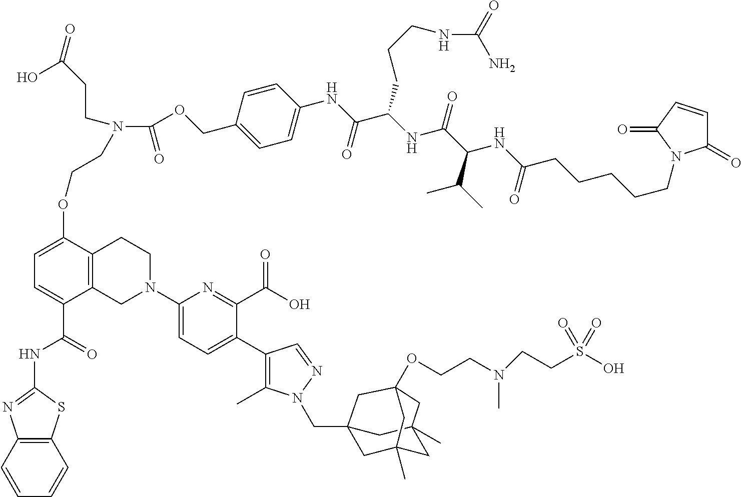 [SMILES: Cc1c(-c2ccc(N3CCc4c(OCCN(CCC(=O)O)C(=O)OCc5ccc(NC(=O)[C@H](CCCNC(N)=O)NC(=O)[C@@H](NC(=O)CCCCCN6C(=O)C=CC6=O)C(C)C)cc5)ccc(C(=O)Nc5nc6ccccc6s5)c4C3)nc2C(=O)O)cnn1CC12CC3(C)CC(C)(C1)CC(OCCN(C)CCS(=O)(=O)O)(C3)C2] |
| 2.52 | JK | 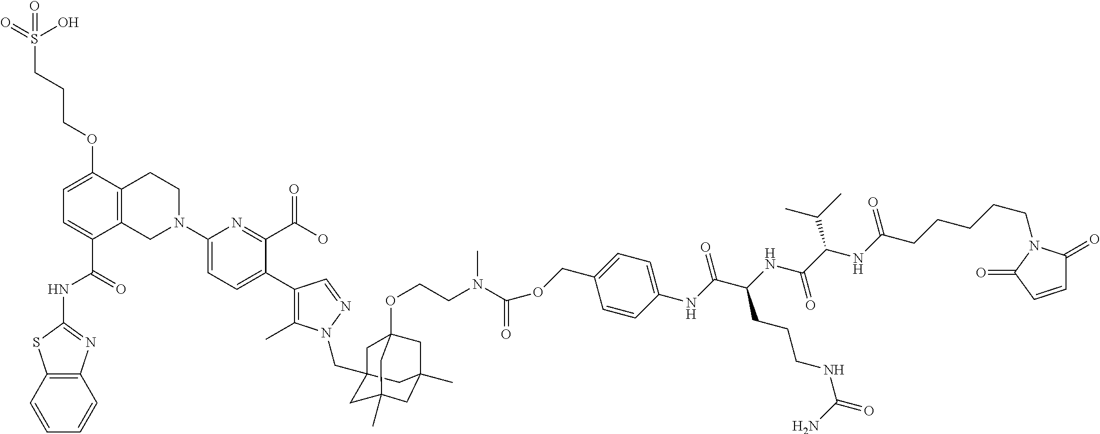 [SMILES: Cc1c(-c2ccc(N3CCc4c(OCCCS(=O)(=O)O)ccc(C(=O)Nc5nc6ccccc6s5)c4C3)nc2C(=O)O)cnn1CC12CC3(C)CC(C)(C1)CC(OCCN(C)C(=O)OCc1ccc(NC(=O)[C@H](CCCNC(N)=O)NC(=O)[C@@H](NC(=O)CCCCCN4C(=O)C=CC4=O)C(C)C)cc1)(C3)C2] |
| 2.53 | JJ | 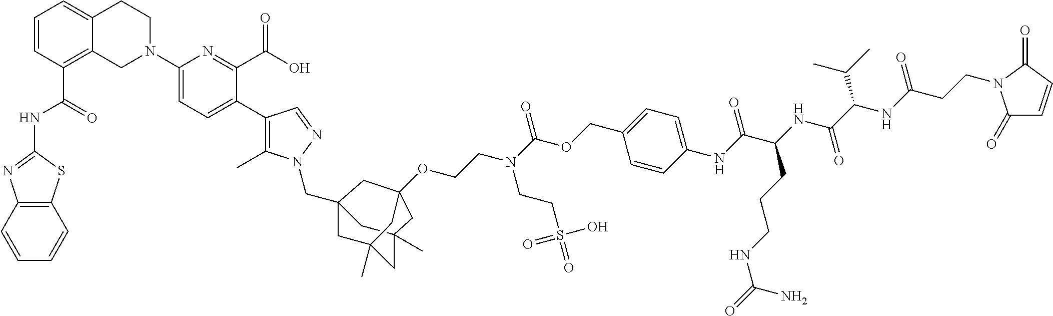 [SMILES: Cc1c(-c2ccc(N3CCc4cccc(C(=O)Nc5nc6ccccc6s5)c4C3)nc2C(=O)O)cnn1CC12CC3(C)CC(C)(C1)CC(OCCN(CCS(=O)(=O)O)C(=O)OCc1ccc(NC(=O)[C@H](CCCNC(N)=O)NC(=O)[C@@H](NC(=O)CCN4C(=O)C=CC4=O)C(C)C)cc1)(C3)C2] |
| 2.54 | JL | 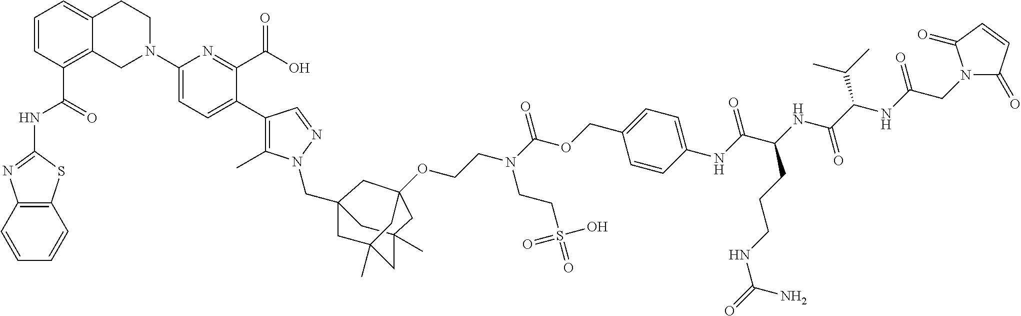 [SMILES: Cc1c(-c2ccc(N3CCc4cccc(C(=O)Nc5nc6ccccc6s5)c4C3)nc2C(=O)O)cnn1CC12CC3(C)CC(C)(C1)CC(OCCN(CCS(=O)(=O)O)C(=O)OCc1ccc(NC(=O)[C@H](CCCNC(N)=O)NC(=O)[C@@H](NC(=O)CN4C(=O)C=CC4=O)C(C)C)cc1)(C3)C2] |
| 2.55 | FE | 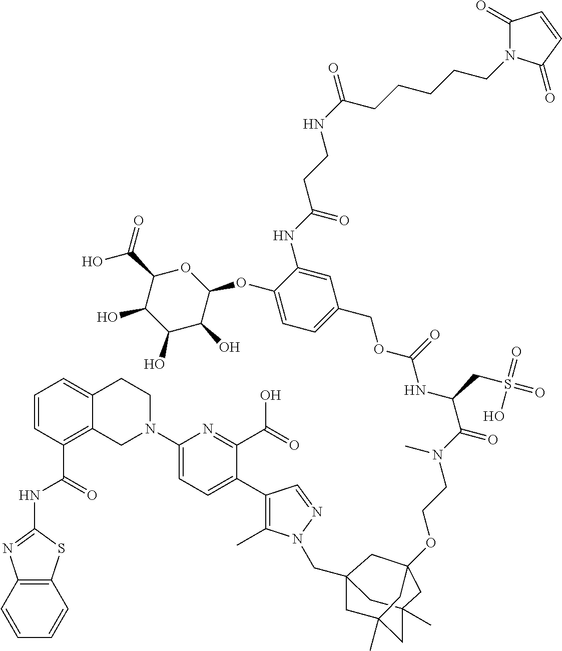 [SMILES: Cc1c(-c2ccc(N3CCc4cccc(C(=O)Nc5nc6ccccc6s5)c4C3)nc2C(=O)O)cnn1CC12CC3(C)CC(C)(C1)CC(OCCN(C)C(=O)[C@H](CS(=O)(=O)O)NC(=O)OCc1ccc(O[C@@H]4O[C@H](C(=O)O)[C@H](O)[C@H](O)[C@@H]4O)c(NC(=O)CCNC(=O)CCCCCN4C(=O)C=CC4=O)c1)(C3)C2] |
| 2.56 | GG | 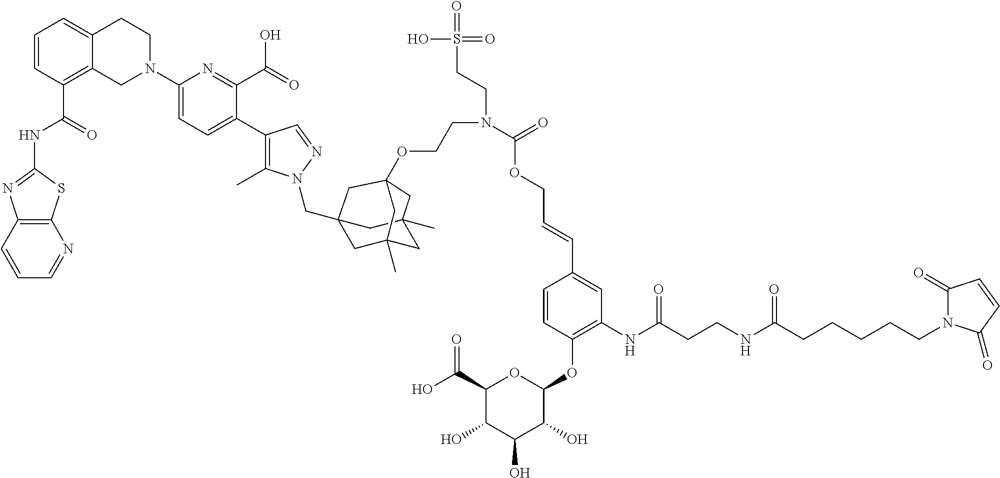 [SMILES: Cc1c(-c2ccc(N3CCc4cccc(C(=O)Nc5nc6cccnc6s5)c4C3)nc2C(=O)O)cnn1CC12CC3(C)CC(C)(C1)CC(OCCN(CCS(=O)(=O)O)C(=O)OC/C=C/c1ccc(O[C@@H]4O[C@H](C(=O)O)[C@@H](O)[C@H](O)[C@H]4O)c(NC(=O)CCNC(=O)CCCCCN4C(=O)C=CC4=O)c1)(C3)C2] |
| 2.57 | GM | 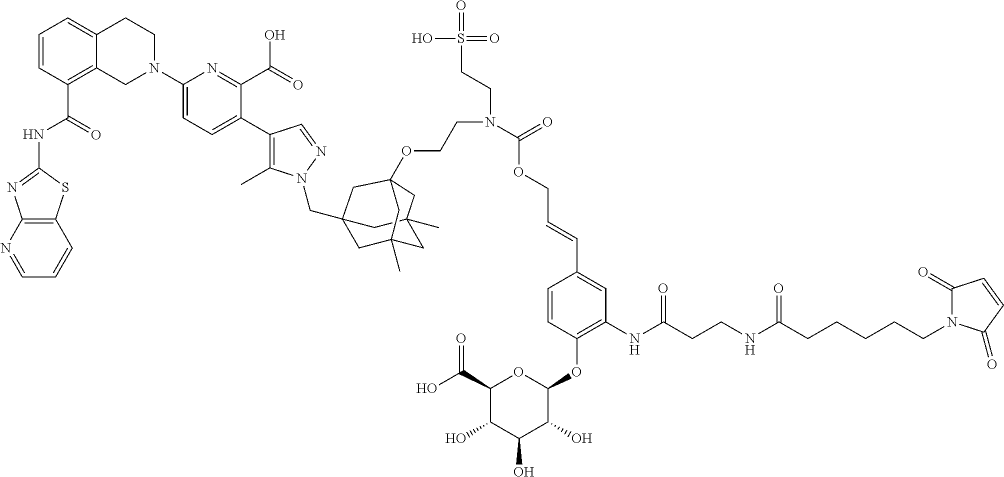 [SMILES: Cc1c(-c2ccc(N3CCc4cccc(C(=O)Nc5nc6ncccc6s5)c4C3)nc2C(=O)O)cnn1CC12CC3(C)CC(C)(C1)CC(OCCN(CCS(=O)(=O)O)C(=O)OC/C=C/c1ccc(O[C@@H]4O[C@H](C(=O)O)[C@@H](O)[C@H](O)[C@H]4O)c(NC(=O)CCNC(=O)CCCCCN4C(=O)C=CC4=O)c1)(C3)C2] |
| 2.58 | HD | 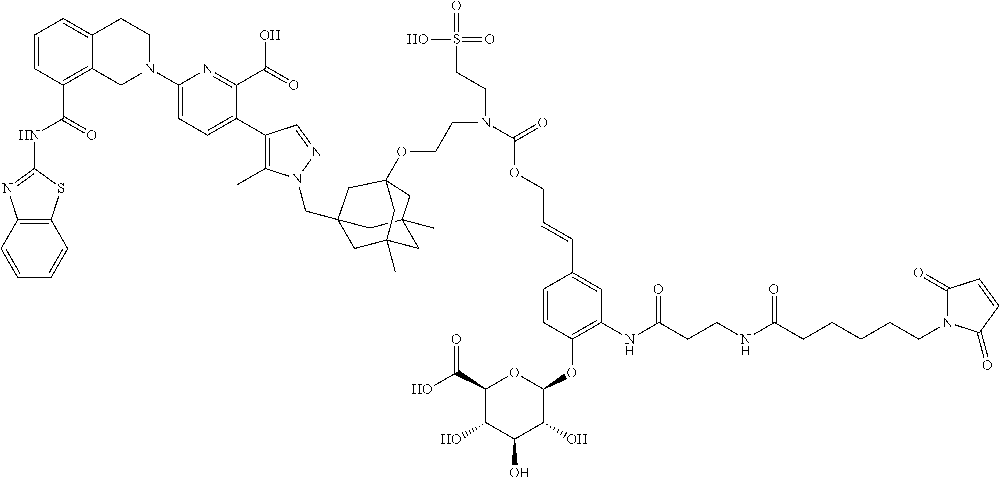 [SMILES: Cc1c(-c2ccc(N3CCc4cccc(C(=O)Nc5nc6ccccc6s5)c4C3)nc2C(=O)O)cnn1CC12CC3(C)CC(C)(C1)CC(OCCN(CCS(=O)(=O)O)C(=O)OC/C=C/c1ccc(O[C@@H]4O[C@H](C(=O)O)[C@@H](O)[C@H](O)[C@H]4O)c(NC(=O)CCNC(=O)CCCCCN4C(=O)C=CC4=O)c1)(C3)C2] |
| 2.59 | HS | 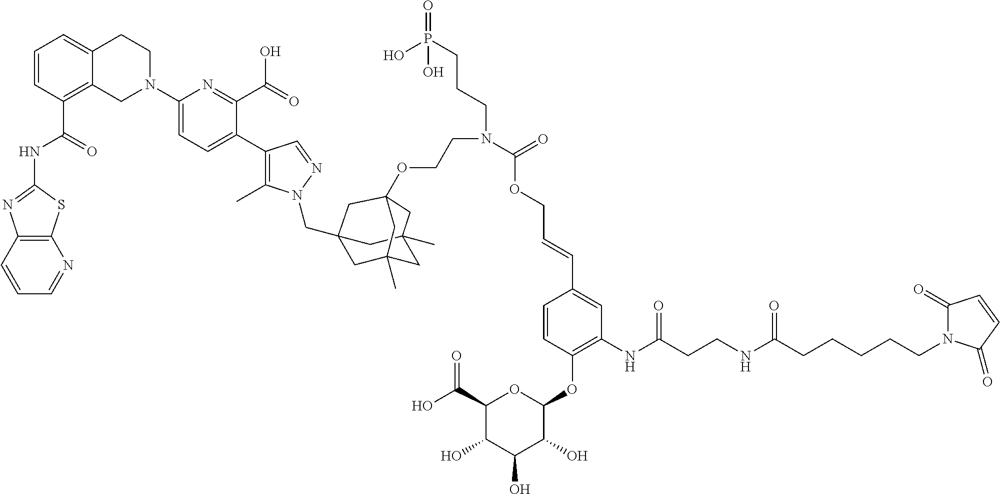 [SMILES: Cc1c(-c2ccc(N3CCc4cccc(C(=O)Nc5nc6cccnc6s5)c4C3)nc2C(=O)O)cnn1CC12CC3(C)CC(C)(C1)CC(OCCN(CCCP(=O)(O)O)C(=O)OC/C=C/c1ccc(O[C@@H]4O[C@H](C(=O)O)[C@@H](O)[C@H](O)[C@H]4O)c(NC(=O)CCNC(=O)CCCCCN4C(=O)C=CC4=O)c1)(C3)C2] |
| 2.60 | HW | 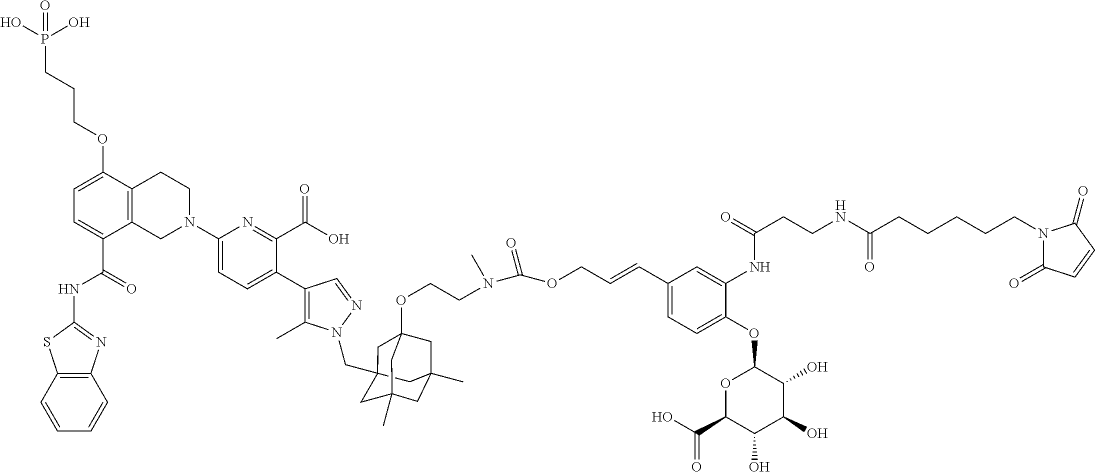 [SMILES: Cc1c(-c2ccc(N3CCc4c(OCCCP(=O)(O)O)ccc(C(=O)Nc5nc6ccccc6s5)c4C3)nc2C(=O)O)cnn1CC12CC3(C)CC(C)(C1)CC(OCCN(C)C(=O)OC/C=C/c1ccc(O[C@@H]4O[C@H](C(=O)O)[C@@H](O)[C@H](O)[C@H]4O)c(NC(=O)CCNC(=O)CCCCCN4C(=O)C=CC4=O)c1)(C3)C2] |
| 2.61 | HX | 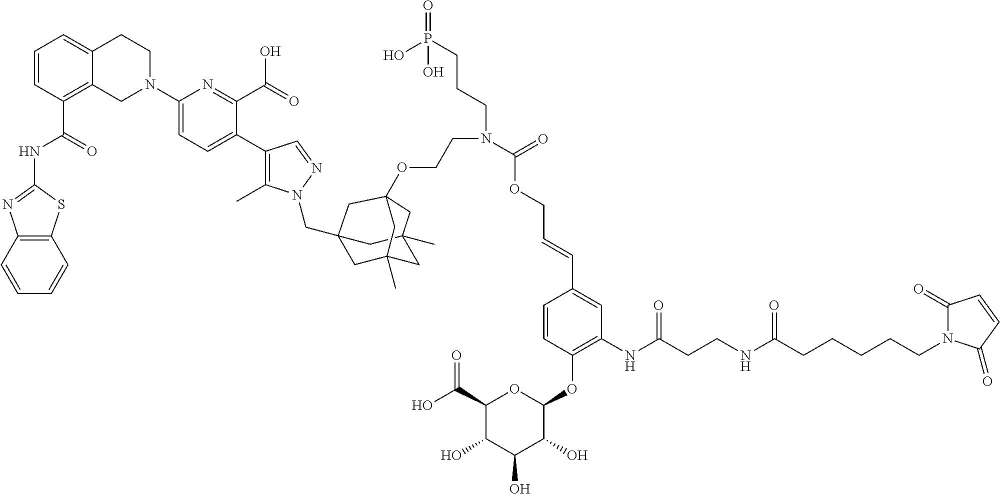 [SMILES: Cc1c(-c2ccc(N3CCc4cccc(C(=O)Nc5nc6ccccc6s5)c4C3)nc2C(=O)O)cnn1CC12CC3(C)CC(C)(C1)CC(OCCN(CCCP(=O)(O)O)C(=O)OC/C=C/c1ccc(O[C@@H]4O[C@H](C(=O)O)[C@@H](O)[C@H](O)[C@H]4O)c(NC(=O)CCNC(=O)CCCCCN4C(=O)C=CC4=O)c1)(C3)C2] |
| 2.62 | HY | 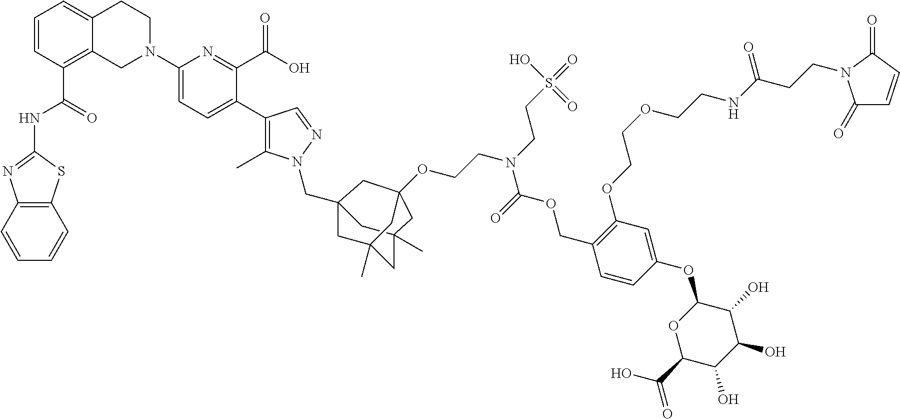 [SMILES: Cc1c(-c2ccc(N3CCc4cccc(C(=O)Nc5nc6ccccc6s5)c4C3)nc2C(=O)O)cnn1CC12CC3(C)CC(C)(C1)CC(OCCN(CCS(=O)(=O)O)C(=O)OCc1ccc(O[C@@H]4O[C@H](C(=O)O)[C@@H](O)[C@H](O)[C@H]4O)cc1OCCOCCNC(=O)CCN1C(=O)C=CC1=O)(C3)C2] |
| 2.63 | IB | 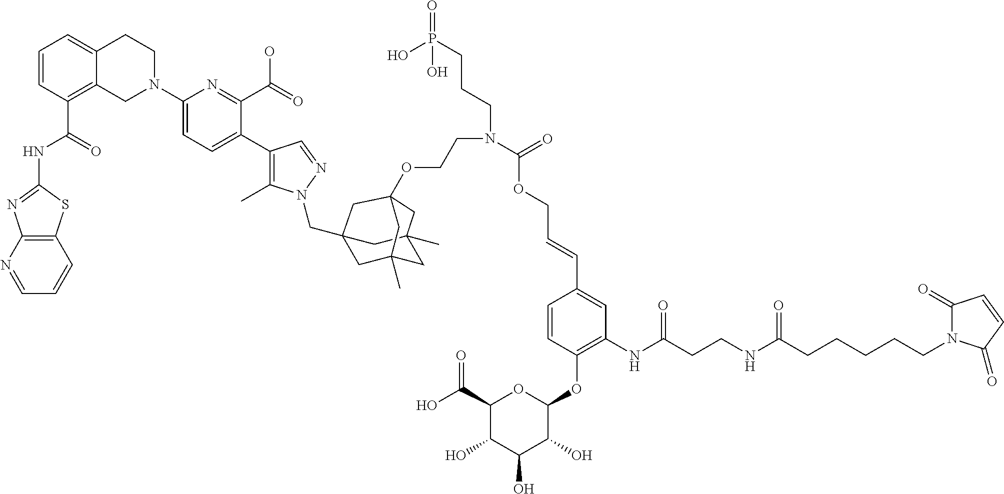 [SMILES: Cc1c(-c2ccc(N3CCc4cccc(C(=O)Nc5nc6ncccc6s5)c4C3)nc2C(=O)O)cnn1CC12CC3(C)CC(C)(C1)CC(OCCN(CCCP(=O)(O)O)C(=O)OC/C=C/c1ccc(O[C@@H]4O[C@H](C(=O)O)[C@@H](O)[C@H](O)[C@H]4O)c(NC(=O)CCNC(=O)CCCCCN4C(=O)C=CC4=O)c1)(C3)C2] |
| 2.64 | IE | 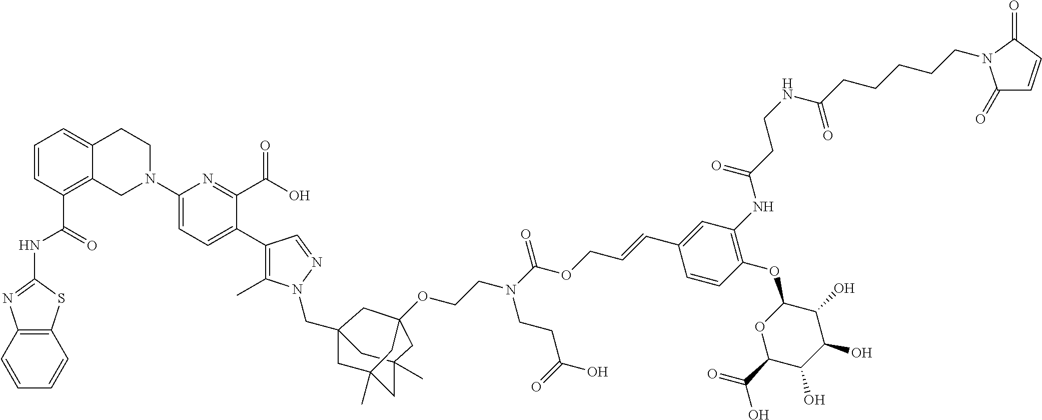 [SMILES: Cc1c(-c2ccc(N3CCc4cccc(C(=O)Nc5nc6ccccc6s5)c4C3)nc2C(=O)O)cnn1CC12CC3(C)CC(C)(C1)CC(OCCN(CCC(=O)O)C(=O)OC/C=C/c1ccc(O[C@@H]4O[C@H](C(=O)O)[C@@H](O)[C@H](O)[C@H]4O)c(NC(=O)CCNC(=O)CCCCCN4C(=O)C=CC4=O)c1)(C3)C2] |
| 2.65 | II | 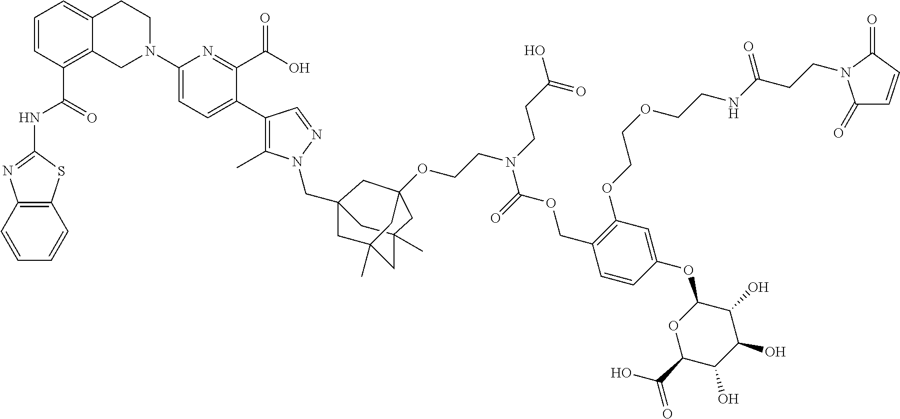 [SMILES: Cc1c(-c2ccc(N3CCc4cccc(C(=O)Nc5nc6ccccc6s5)c4C3)nc2C(=O)O)cnn1CC12CC3(C)CC(C)(C1)CC(OCCN(CCC(=O)O)C(=O)OCc1ccc(O[C@@H]4O[C@H](C(=O)O)[C@@H](O)[C@H](O)[C@H]4O)cc1OCCOCCNC(=O)CCN1C(=O)C=CC1=O)(C3)C2] |
| 2.66 | KY | 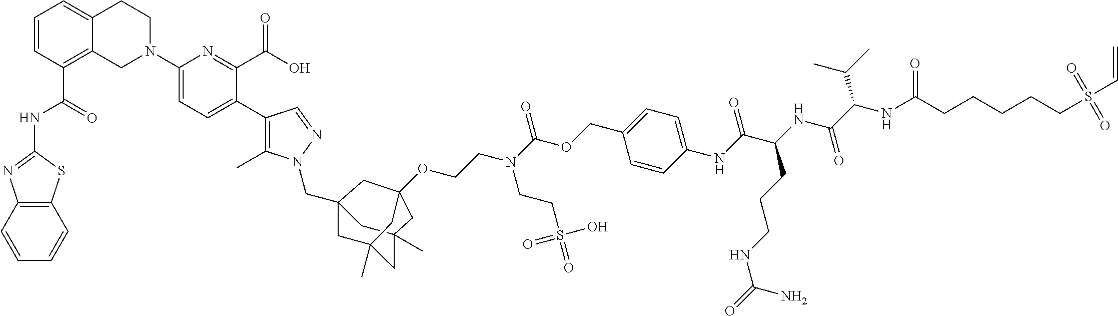 [SMILES: C=CS(=O)(=O)CCCCCC(=O)N[C@H](C(=O)N[C@@H](CCCNC(N)=O)C(=O)Nc1ccc(COC(=O)N(CCOC23CC4(C)CC(C)(CC(Cn5ncc(-c6ccc(N7CCc8cccc(C(=O)Nc9nc%10ccccc%10s9)c8C7)nc6C(=O)O)c5C)(C4)C2)C3)CCS(=O)(=O)O)cc1)C(C)C] |
| 2.67 | IW | 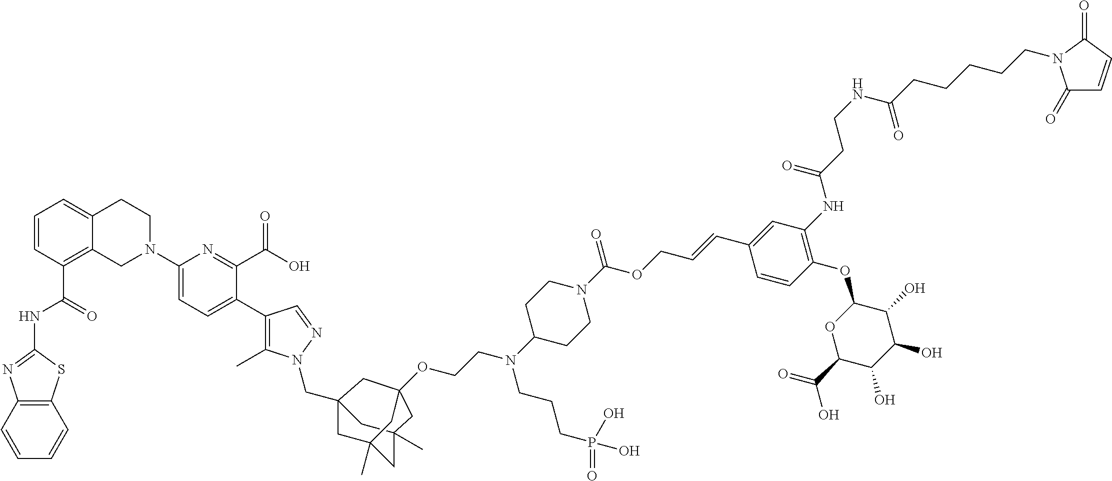 [SMILES: Cc1c(-c2ccc(N3CCc4cccc(C(=O)Nc5nc6ccccc6s5)c4C3)nc2C(=O)O)cnn1CC12CC3(C)CC(C)(C1)CC(OCCN(CCCP(=O)(O)O)C1CCN(C(=O)OC/C=C/c4ccc(O[C@@H]5O[C@H](C(=O)O)[C@@H](O)[C@H](O)[C@H]5O)c(NC(=O)CCNC(=O)CCCCCN5C(=O)C=CC5=O)c4)CC1)(C3)C2] |
| 2.68 | IY | 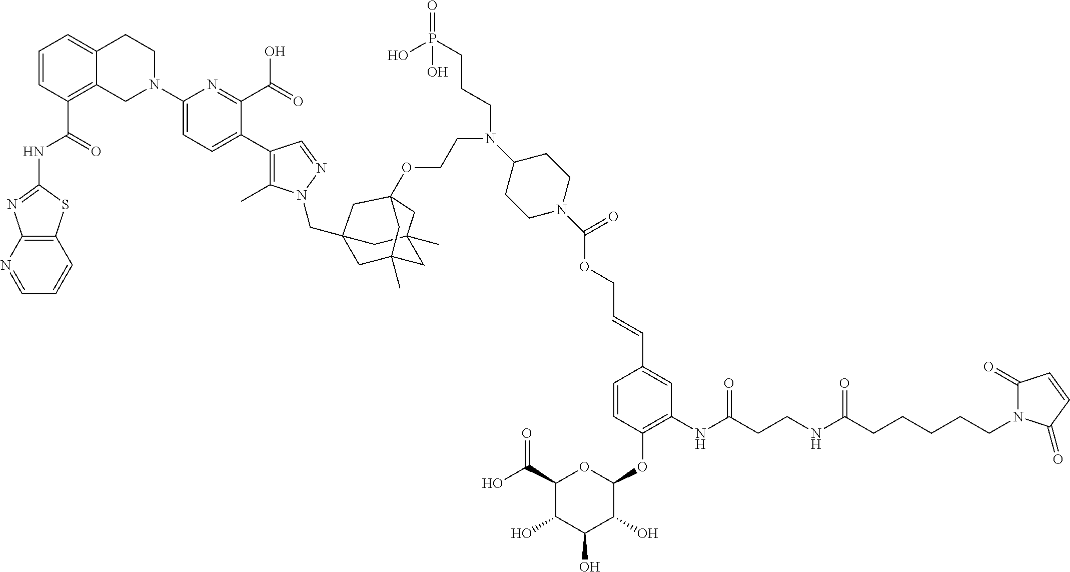 [SMILES: Cc1c(-c2ccc(N3CCc4cccc(C(=O)Nc5nc6ncccc6s5)c4C3)nc2C(=O)O)cnn1CC12CC3(C)CC(C)(C1)CC(OCCN(CCCP(=O)(O)O)C1CCN(C(=O)OC/C=C/c4ccc(O[C@@H]5O[C@H](C(=O)O)[C@@H](O)[C@H](O)[C@H]5O)c(NC(=O)CCNC(=O)CCCCCN5C(=O)C=CC5=O)c4)CC1)(C3)C2] |
| 2.69 | JA | 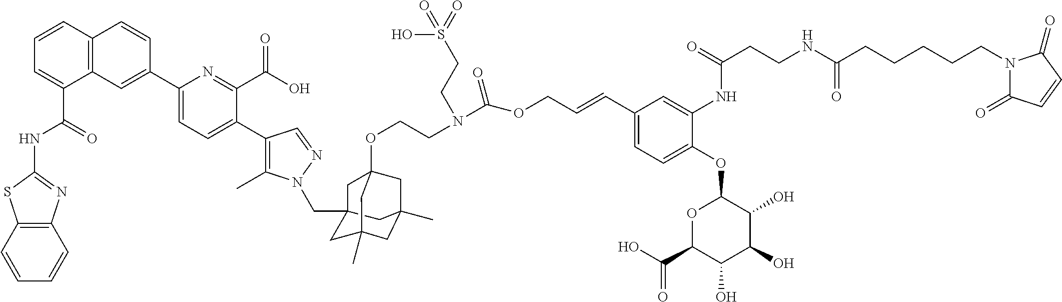 [SMILES: Cc1c(-c2ccc(-c3ccc4cccc(C(=O)Nc5nc6ccccc6s5)c4c3)nc2C(=O)O)cnn1CC12CC3(C)CC(C)(C1)CC(OCCN(CCS(=O)(=O)O)C(=O)OC/C=C/c1ccc(O[C@@H]4O[C@H](C(=O)O)[C@@H](O)[C@H](O)[C@H]4O)c(NC(=O)CCNC(=O)CCCCCN4C(=O)C=CC4=O)c1)(C3)C2] |
| 2.77 | FA | 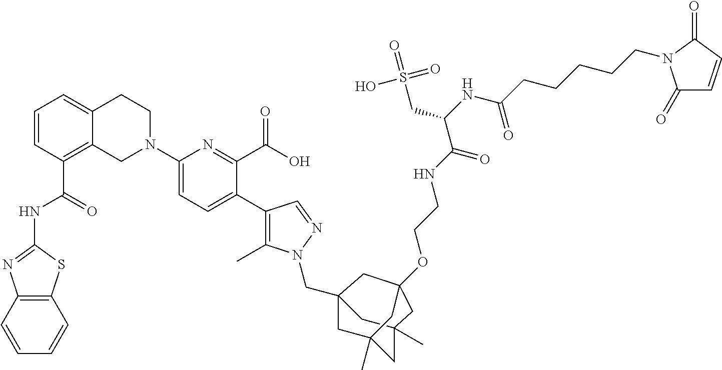 [SMILES: Cc1c(-c2ccc(N3CCc4cccc(C(=O)Nc5nc6ccccc6s5)c4C3)nc2C(=O)O)cnn1CC12CC3(C)CC(C)(C1)CC(OCCNC(=O)[C@H](CS(=O)(=O)O)NC(=O)CCCCCN1C(=O)C=CC1=O)(C3)C2] |
| 2.78 | FJ | 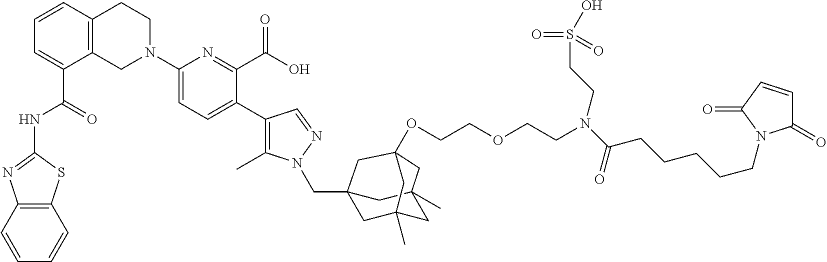 [SMILES: Cc1c(-c2ccc(N3CCc4cccc(C(=O)Nc5nc6ccccc6s5)c4C3)nc2C(=O)O)cnn1CC12CC3(C)CC(C)(C1)CC(OCCOCCN(CCS(=O)(=O)O)C(=O)CCCCCN1C(=O)C=CC1=O)(C3)C2] |
| 2.79 | FK | 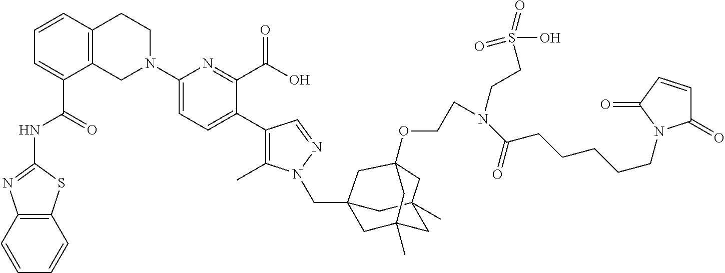 [SMILES: Cc1c(-c2ccc(N3CCc4cccc(C(=O)Nc5nc6ccccc6s5)c4C3)nc2C(=O)O)cnn1CC12CC3(C)CC(C)(C1)CC(OCCN(CCS(=O)(=O)O)C(=O)CCCCCN1C(=O)C=CC1=O)(C3)C2] |
| 2.80 | FQ | 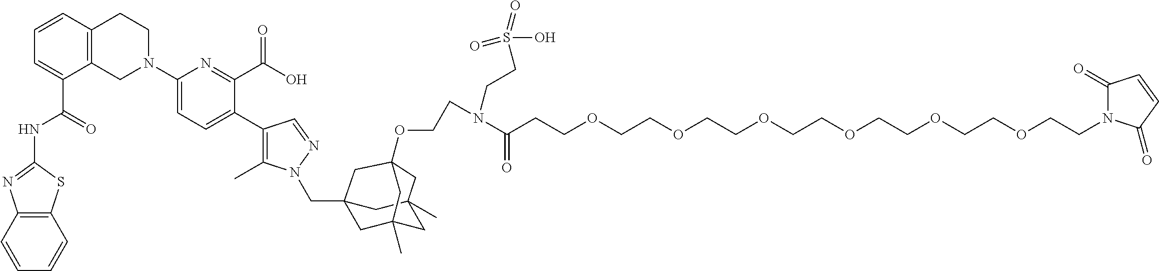 [SMILES: Cc1c(-c2ccc(N3CCc4cccc(C(=O)Nc5nc6ccccc6s5)c4C3)nc2C(=O)O)cnn1CC12CC3(C)CC(C)(C1)CC(OCCN(CCS(=O)(=O)O)C(=O)CCOCCOCCOCCOCCOCCOCCN1C(=O)C=CC1=O)(C3)C2] |
| 2.81 | FR | 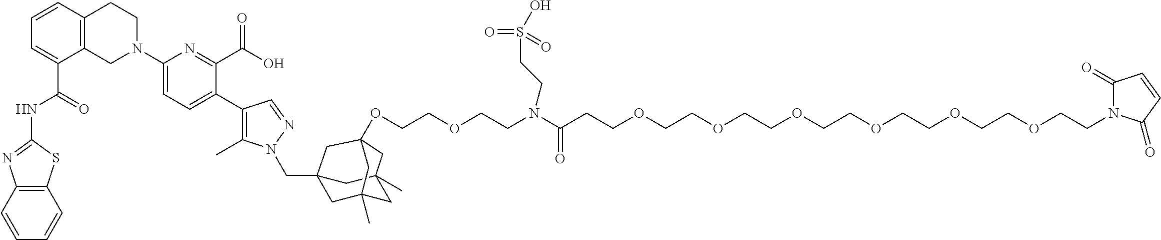 [SMILES: Cc1c(-c2ccc(N3CCc4cccc(C(=O)Nc5nc6ccccc6s5)c4C3)nc2C(=O)O)cnn1CC12CC3(C)CC(C)(C1)CC(OCCOCCN(CCS(=O)(=O)O)C(=O)CCOCCOCCOCCOCCOCCOCCN1C(=O)C=CC1=O)(C3)C2] |
| 2.82 | JE | 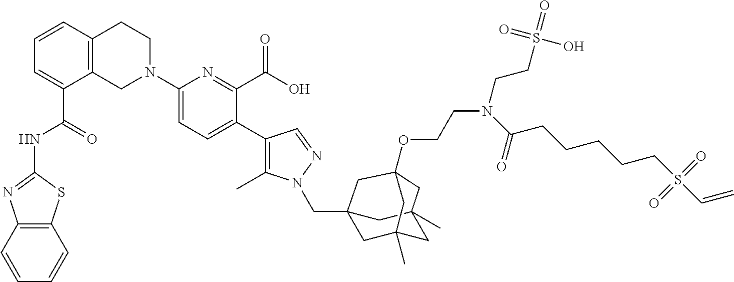 [SMILES: C=CS(=O)(=O)CCCCCC(=O)N(CCOC12CC3(C)CC(C)(CC(Cn4ncc(-c5ccc(N6CCc7cccc(C(=O)Nc8nc9ccccc9s8)c7C6)nc5C(=O)O)c4C)(C3)C1)C2)CCS(=O)(=O)O] |
| 2.83 | JM | 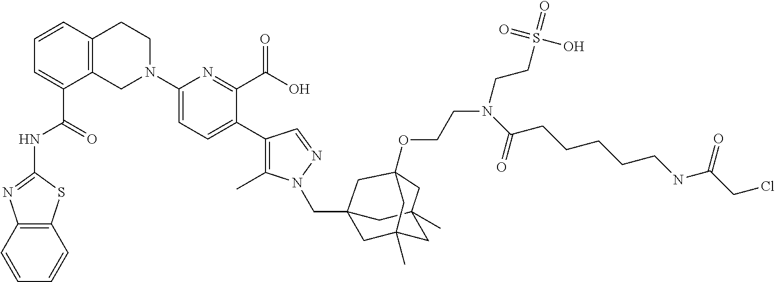 [SMILES: Cc1c(-c2ccc(N3CCc4cccc(C(=O)Nc5nc6ccccc6s5)c4C3)nc2C(=O)O)cnn1CC12CC3(C)CC(C)(C1)CC(OCCN(CCS(=O)(=O)O)C(=O)CCCCCNC(=O)CCl)(C3)C2] |
| 2.84 | LE | 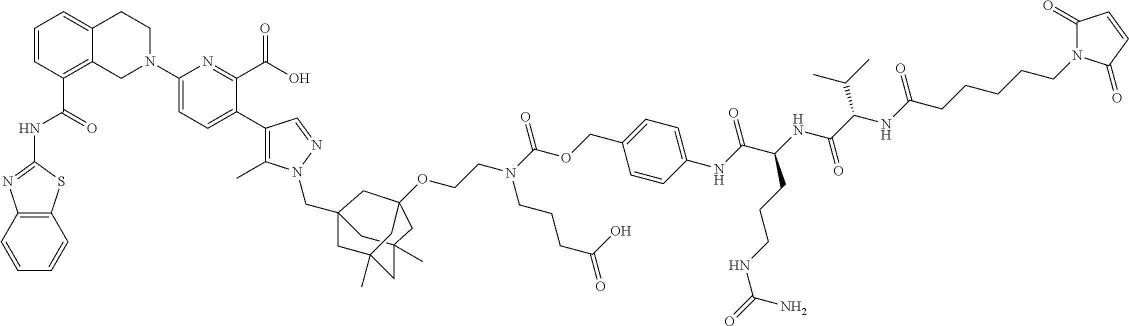 [SMILES: Cc1c(-c2ccc(N3CCc4cccc(C(=O)Nc5nc6ccccc6s5)c4C3)nc2C(=O)O)cnn1CC12CC3(C)CC(C)(C1)CC(OCCN(CCCC(=O)O)C(=O)OCc1ccc(NC(=O)[C@H](CCCNC(N)=O)NC(=O)[C@@H](NC(=O)CCCCCN4C(=O)C=CC4=O)C(C)C)cc1)(C3)C2] |
| 2.85 | LH | 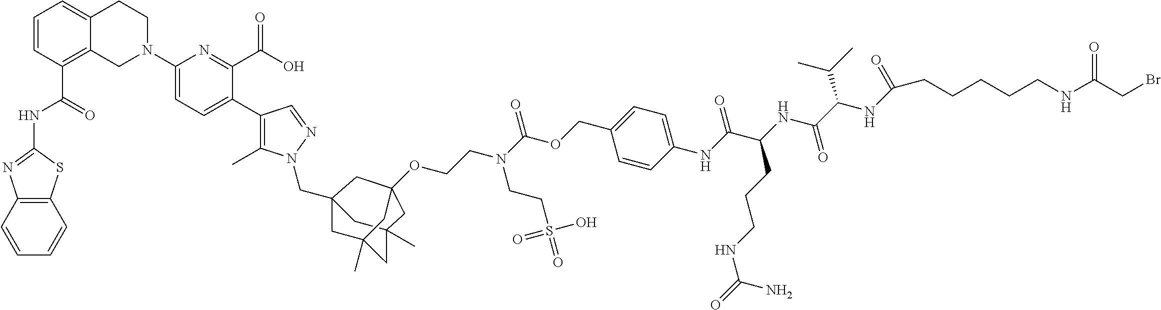 [SMILES: Cc1c(-c2ccc(N3CCc4cccc(C(=O)Nc5nc6ccccc6s5)c4C3)nc2C(=O)O)cnn1CC12CC3(C)CC(C)(C1)CC(OCCN(CCS(=O)(=O)O)C(=O)OCc1ccc(NC(=O)[C@H](CCCNC(N)=O)NC(=O)[C@@H](NC(=O)CCCCCNC(=O)CBr)C(C)C)cc1)(C3)C2] |
| 2.86 | LJ | 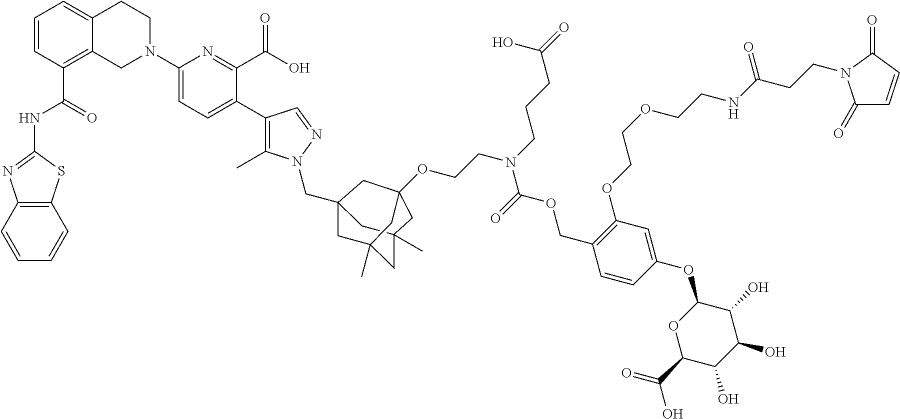 [SMILES: Cc1c(-c2ccc(N3CCc4cccc(C(=O)Nc5nc6ccccc6s5)c4C3)nc2C(=O)O)cnn1CC12CC3(C)CC(C)(C1)CC(OCCN(CCCC(=O)O)C(=O)OCc1ccc(O[C@@H]4O[C@H](C(=O)O)[C@@H](O)[C@H](O)[C@H]4O)cc1OCCOCCNC(=O)CCN1C(=O)C=CC1=O)(C3)C2] |
| 2.87 | MA | 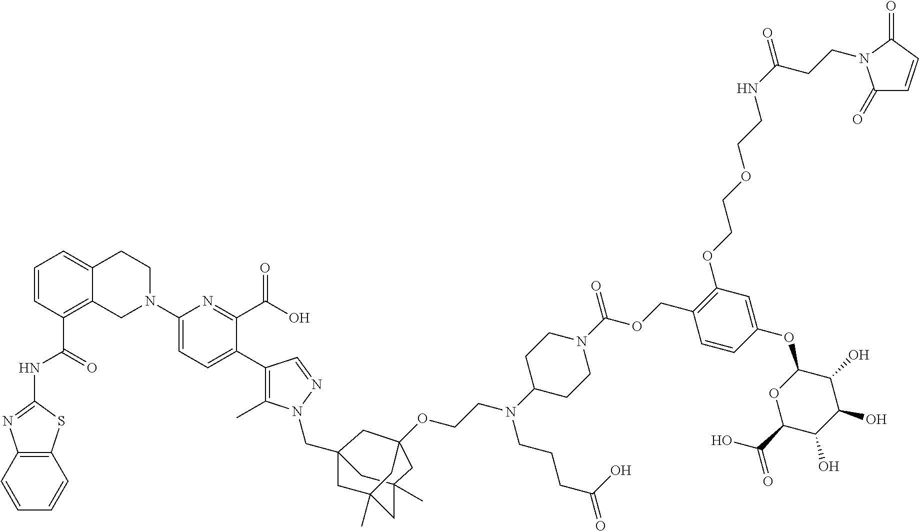 [SMILES: Cc1c(-c2ccc(N3CCc4cccc(C(=O)Nc5nc6ccccc6s5)c4C3)nc2C(=O)O)cnn1CC12CC3(C)CC(C)(C1)CC(OCCN(CCCC(=O)O)C1CCN(C(=O)OCc4ccc(O[C@@H]5O[C@H](C(=O)O)[C@@H](O)[C@H](O)[C@H]5O)cc4OCCOCCNC(=O)CCN4C(=O)C=CC4=O)CC1)(C3)C2] |
| 2.88 | MD | 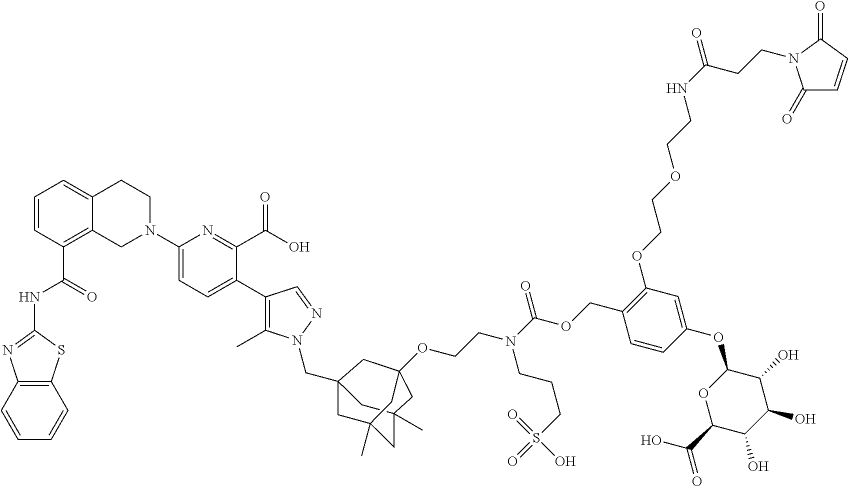 [SMILES: Cc1c(-c2ccc(N3CCc4cccc(C(=O)Nc5nc6ccccc6s5)c4C3)nc2C(=O)O)cnn1CC12CC3(C)CC(C)(C1)CC(OCCN(CCCS(=O)(=O)O)C(=O)OCc1ccc(O[C@@H]4O[C@H](C(=O)O)[C@@H](O)[C@H](O)[C@H]4O)cc1OCCOCCNC(=O)CCN1C(=O)C=CC1=O)(C3)C2] |
| 2.89 | MG | 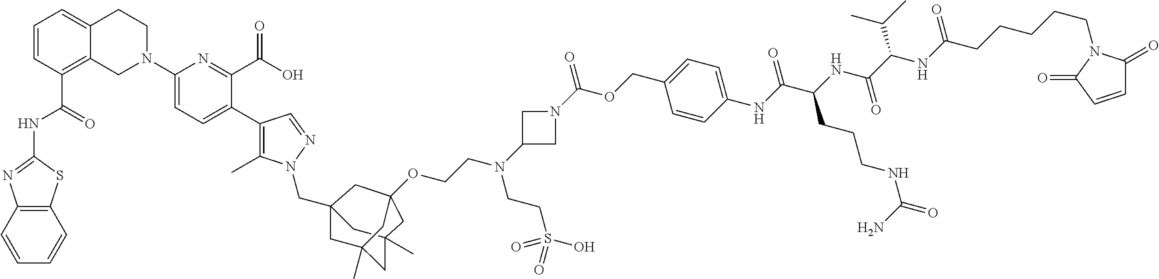 [SMILES: Cc1c(-c2ccc(N3CCc4cccc(C(=O)Nc5nc6ccccc6s5)c4C3)nc2C(=O)O)cnn1CC12CC3(C)CC(C)(C1)CC(OCCN(CCS(=O)(=O)O)C1CN(C(=O)OCc4ccc(NC(=O)[C@H](CCCNC(N)=O)NC(=O)[C@@H](NC(=O)CCCCCN5C(=O)C=CC5=O)C(C)C)cc4)C1)(C3)C2] |
| 2.90 | MS | 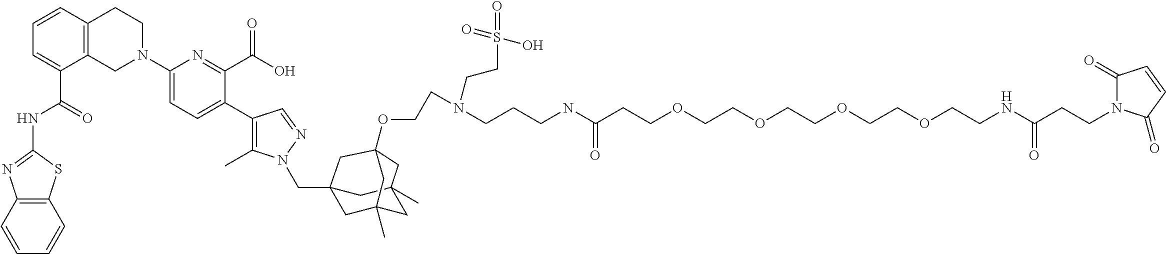 [SMILES: Cc1c(-c2ccc(N3CCc4cccc(C(=O)Nc5nc6ccccc6s5)c4C3)nc2C(=O)O)cnn1CC12CC3(C)CC(C)(C1)CC(OCCN(CCCNC(=O)CCOCCOCCOCCOCCNC(=O)CCN1C(=O)C=CC1=O)CCS(=O)(=O)O)(C3)C2] |
| 2.91 | MR | 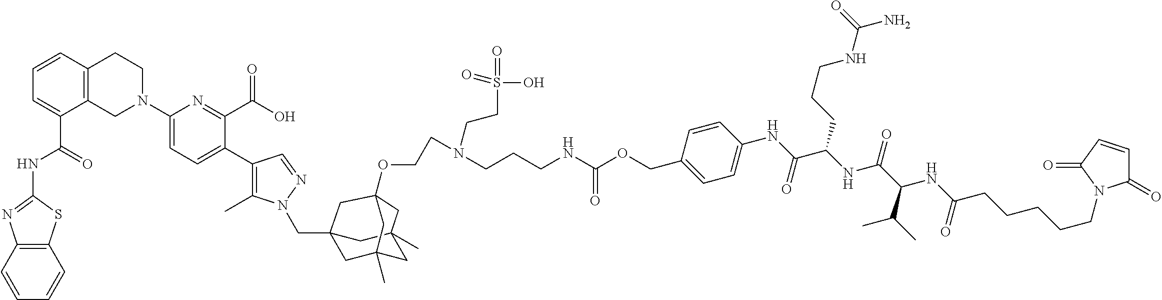 [SMILES: Cc1c(-c2ccc(N3CCc4cccc(C(=O)Nc5nc6ccccc6s5)c4C3)nc2C(=O)O)cnn1CC12CC3(C)CC(C)(C1)CC(OCCN(CCCNC(=O)OCc1ccc(NC(=O)[C@H](CCCNC(N)=O)NC(=O)[C@@H](NC(=O)CCCCCN4C(=O)C=CC4=O)C(C)C)cc1)CCS(=O)(=O)O)(C3)C2] |
| 2.92 | MQ | 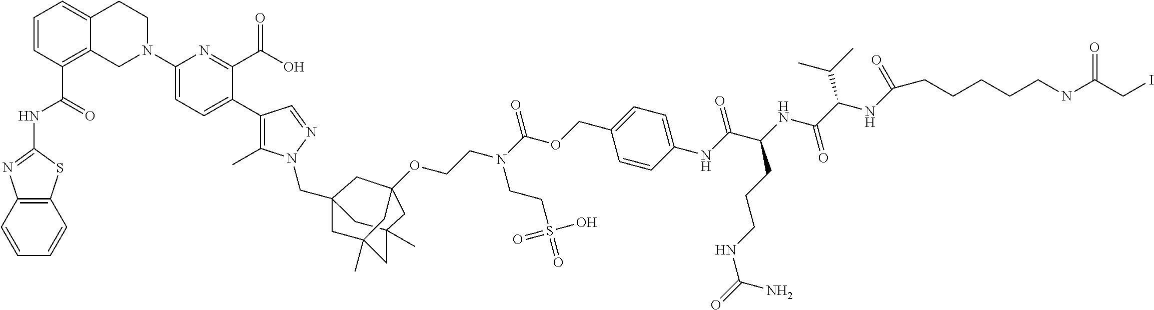 [SMILES: Cc1c(-c2ccc(N3CCc4cccc(C(=O)Nc5nc6ccccc6s5)c4C3)nc2C(=O)O)cnn1CC12CC3(C)CC(C)(C1)CC(OCCN(CCS(=O)(=O)O)C(=O)OCc1ccc(NC(=O)[C@H](CCCNC(N)=O)NC(=O)[C@@H](NC(=O)CCCCCNC(=O)CI)C(C)C)cc1)(C3)C2] |
| 2.93 | MZ | 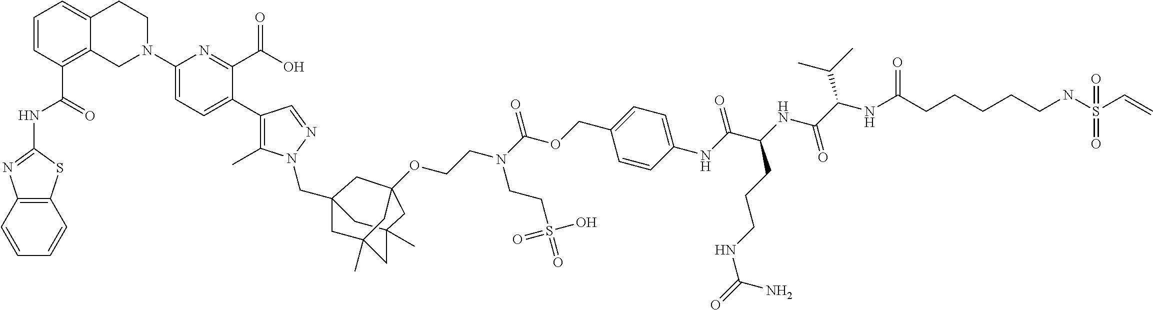 [SMILES: C=CS(=O)(=O)NCCCCCC(=O)N[C@H](C(=O)N[C@@H](CCCNC(N)=O)C(=O)Nc1ccc(COC(=O)N(CCOC23CC4(C)CC(C)(CC(Cn5ncc(-c6ccc(N7CCc8cccc(C(=O)Nc9nc%10ccccc%10s9)c8C7)nc6C(=O)O)c5C)(C4)C2)C3)CCS(=O)(=O)O)cc1)C(C)C] |
| 2.94 | NA | 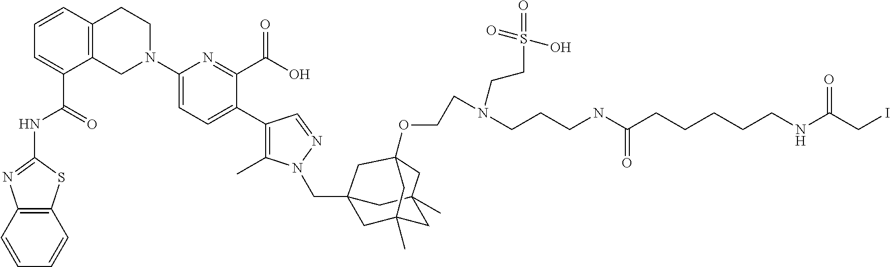 [SMILES: Cc1c(-c2ccc(N3CCc4cccc(C(=O)Nc5nc6ccccc6s5)c4C3)nc2C(=O)O)cnn1CC12CC3(C)CC(C)(C1)CC(OCCN(CCCNC(=O)CCCCCNC(=O)CI)CCS(=O)(=O)O)(C3)C2] |
| 2.95 | NB | 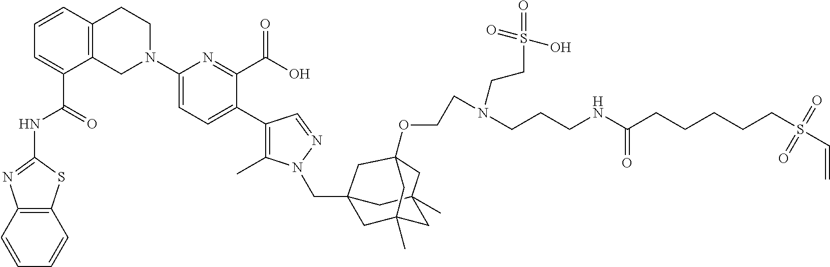 [SMILES: C=CS(=O)(=O)CCCCCC(=O)NCCCN(CCOC12CC3(C)CC(C)(CC(Cn4ncc(-c5ccc(N6CCc7cccc(C(=O)Nc8nc9ccccc9s8)c7C6)nc5C(=O)O)c4C)(C3)C1)C2)CCS(=O)(=O)O] |
| 2.96 | NP | 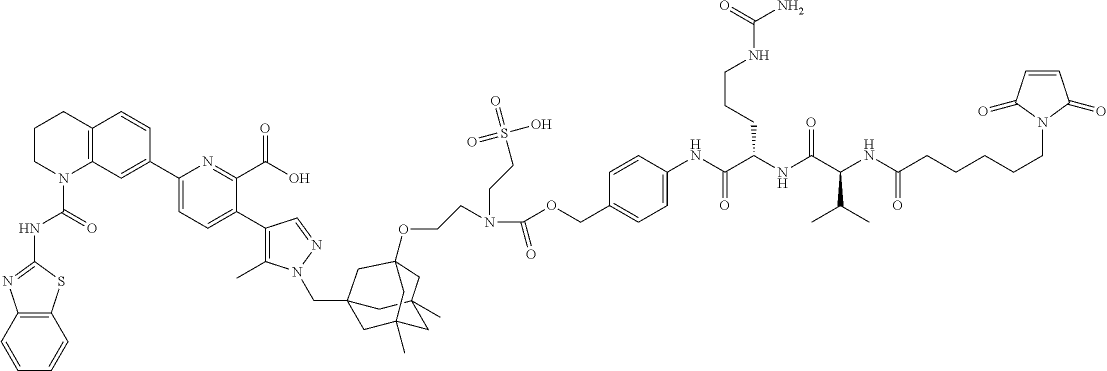 [SMILES: Cc1c(-c2ccc(-c3ccc4c(c3)N(C(=O)Nc3nc5ccccc5s3)CCC4)nc2C(=O)O)cnn1CC12CC3(C)CC(C)(C1)CC(OCCN(CCS(=O)(=O)O)C(=O)OCc1ccc(NC(=O)[C@H](CCCNC(N)=O)NC(=O)[C@@H](NC(=O)CCCCCN4C(=O)C=CC4=O)C(C)C)cc1)(C3)C2] |
| 2.97 | NN | 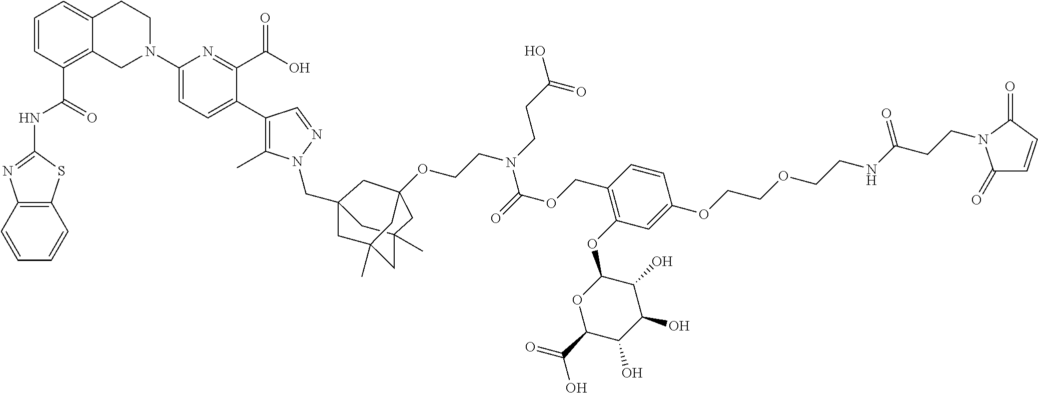 [SMILES: Cc1c(-c2ccc(N3CCc4cccc(C(=O)Nc5nc6ccccc6s5)c4C3)nc2C(=O)O)cnn1CC12CC3(C)CC(C)(C1)CC(OCCN(CCC(=O)O)C(=O)OCc1ccc(OCCOCCNC(=O)CCN4C(=O)C=CC4=O)cc1O[C@@H]1O[C@H](C(=O)O)[C@@H](O)[C@H](O)[C@H]1O)(C3)C2] |
| 2.98 | NO | 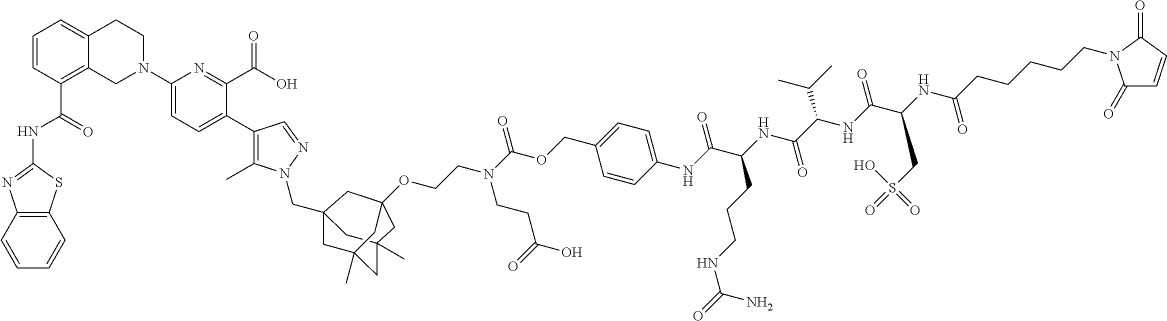 [SMILES: Cc1c(-c2ccc(N3CCc4cccc(C(=O)Nc5nc6ccccc6s5)c4C3)nc2C(=O)O)cnn1CC12CC3(C)CC(C)(C1)CC(OCCN(CCC(=O)O)C(=O)OCc1ccc(NC(=O)[C@H](CCCNC(N)=O)NC(=O)[C@@H](NC(=O)[C@H](CS(=O)(=O)O)NC(=O)CCCCCN4C(=O)C=CC4=O)C(C)C)cc1)(C3)C2] |
| 2.101 | OK | 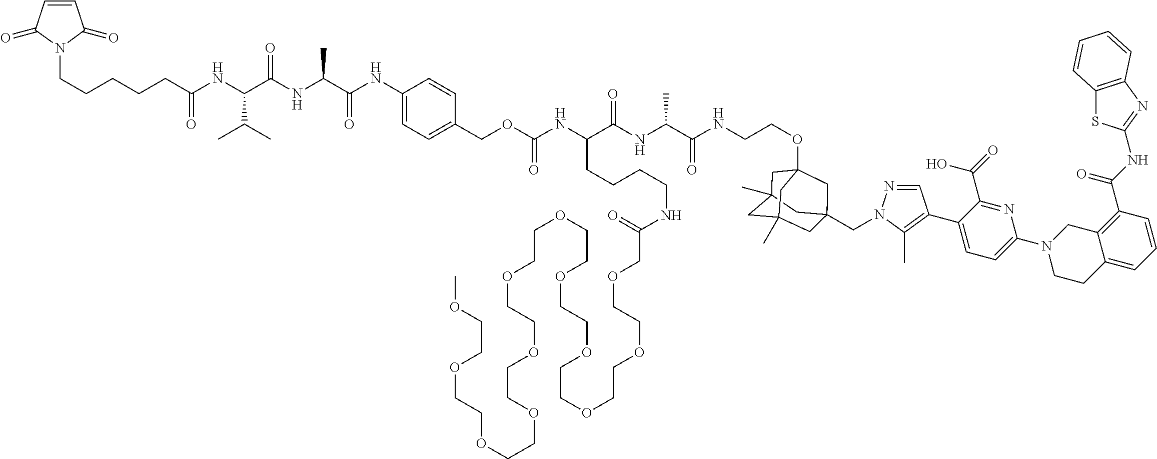 [SMILES: COCCOCCOCCOCCOCCOCCOCCOCCOCCOCCOCCOCC(=O)NCCCCC(NC(=O)OCc1ccc(NC(=O)[C@H](C)NC(=O)[C@@H](NC(=O)CCCCCN2C(=O)C=CC2=O)C(C)C)cc1)C(=O)N[C@H](C)C(=O)NCCOC12CC3(C)CC(C)(CC(Cn4ncc(-c5ccc(N6CCc7cccc(C(=O)Nc8nc9ccccc9s8)c7C6)nc5C(=O)O)c4C)(C3)C1)C2] |
| 2.102 | OW | 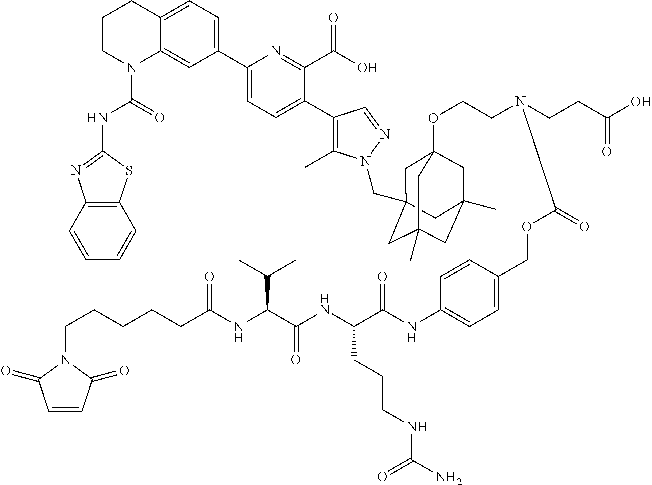 [SMILES: Cc1c(-c2ccc(-c3ccc4c(c3)N(C(=O)Nc3nc5ccccc5s3)CCC4)nc2C(=O)O)cnn1CC12CC3(C)CC(C)(C1)CC(OCCN(CCC(=O)O)C(=O)OCc1ccc(NC(=O)[C@H](CCCNC(N)=O)NC(=O)[C@@H](NC(=O)CCCCCN4C(=O)C=CC4=O)C(C)C)cc1)(C3)C2] |
| 2.103 | PC | 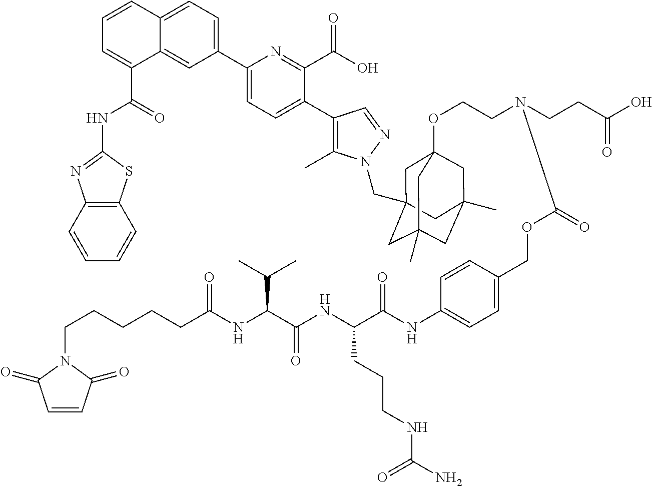 [SMILES: Cc1c(-c2ccc(-c3ccc4cccc(C(=O)Nc5nc6ccccc6s5)c4c3)nc2C(=O)O)cnn1CC12CC3(C)CC(C)(C1)CC(OCCN(CCC(=O)O)C(=O)OCc1ccc(NC(=O)[C@H](CCCNC(N)=O)NC(=O)[C@@H](NC(=O)CCCCCN4C(=O)C=CC4=O)C(C)C)cc1)(C3)C2] |
| 2.104 | PI | 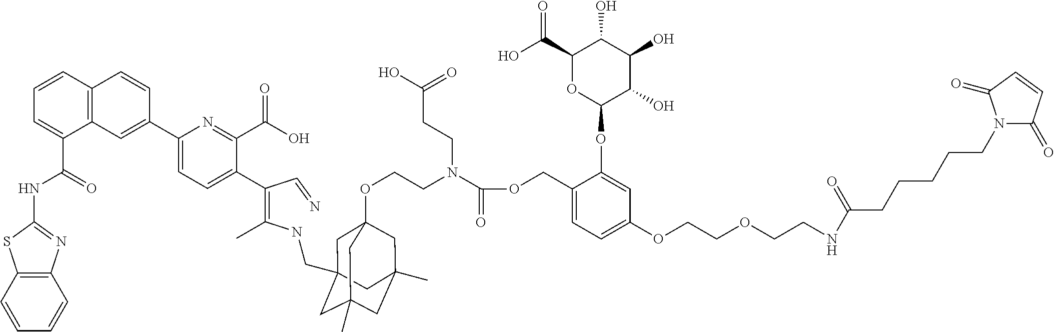 [SMILES: C/C(NCC12CC3(C)CC(C)(C1)CC(OCCN(CCC(=O)O)C(=O)OCc1ccc(OCCOCCNC(=O)CCCCCN4C(=O)C=CC4=O)cc1O[C@H]1O[C@@H](C(=O)O)[C@H](O)[C@@H](O)[C@@H]1O)(C3)C2)=C(/C=N)c1ccc(-c2ccc3cccc(C(=O)Nc4nc5ccccc5s4)c3c2)nc1C(=O)O] |
| 2.105 | PJ | 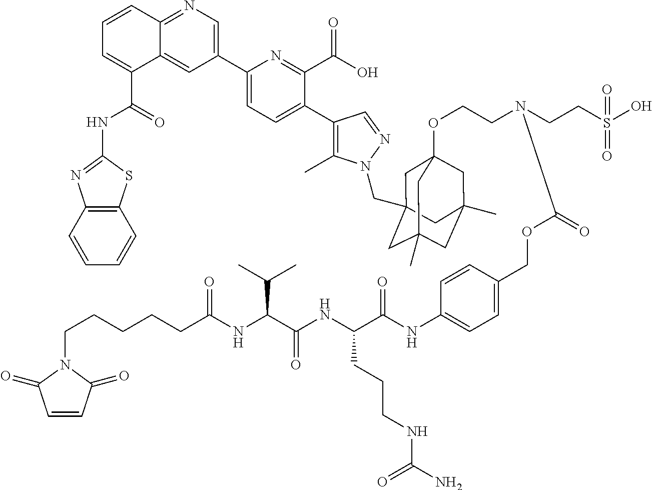 [SMILES: Cc1c(-c2ccc(-c3cnc4cccc(C(=O)Nc5nc6ccccc6s5)c4c3)nc2C(=O)O)cnn1CC12CC3(C)CC(C)(C1)CC(OCCN(CCS(=O)(=O)O)C(=O)OCc1ccc(NC(=O)[C@H](CCCNC(N)=O)NC(=O)[C@@H](NC(=O)CCCCCN4C(=O)C=CC4=O)C(C)C)cc1)(C3)C2] |
| 2.106 | PU | 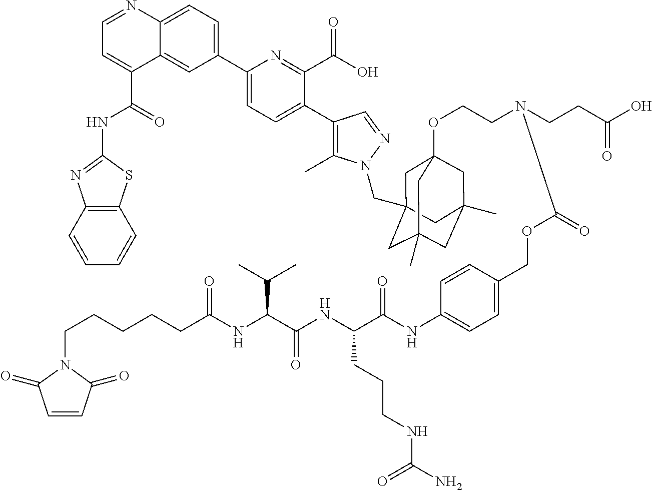 [SMILES: Cc1c(-c2ccc(-c3ccc4nccc(C(=O)Nc5nc6ccccc6s5)c4c3)nc2C(=O)O)cnn1CC12CC3(C)CC(C)(C1)CC(OCCN(CCC(=O)O)C(=O)OCc1ccc(NC(=O)[C@H](CCCNC(N)=O)NC(=O)[C@@H](NC(=O)CCCCCN4C(=O)C=CC4=O)C(C)C)cc1)(C3)C2] |
| 2.107 | PV | 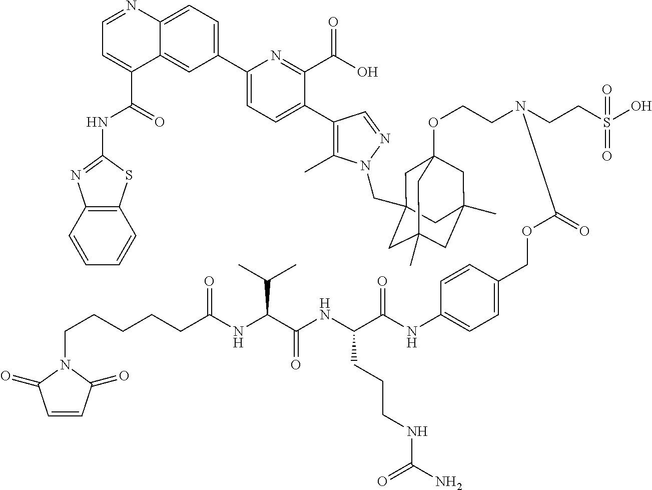 [SMILES: Cc1c(-c2ccc(-c3ccc4nccc(C(=O)Nc5nc6ccccc6s5)c4c3)nc2C(=O)O)cnn1CC12CC3(C)CC(C)(C1)CC(OCCN(CCS(=O)(=O)O)C(=O)OCc1ccc(NC(=O)[C@H](CCCNC(N)=O)NC(=O)[C@@H](NC(=O)CCCCCN4C(=O)C=CC4=O)C(C)C)cc1)(C3)C2] |
| 2.108 | PW | 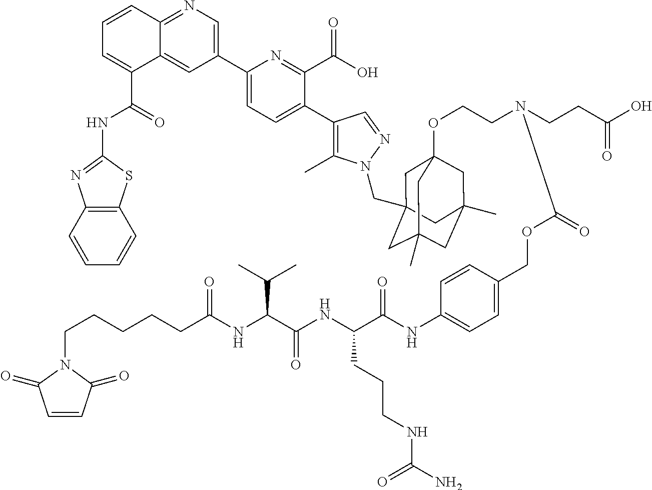 [SMILES: Cc1c(-c2ccc(-c3cnc4cccc(C(=O)Nc5nc6ccccc6s5)c4c3)nc2C(=O)O)cnn1CC12CC3(C)CC(C)(C1)CC(OCCN(CCC(=O)O)C(=O)OCc1ccc(NC(=O)[C@H](CCCNC(N)=O)NC(=O)[C@@H](NC(=O)CCCCCN4C(=O)C=CC4=O)C(C)C)cc1)(C3)C2] |
| 2.109 | QW | 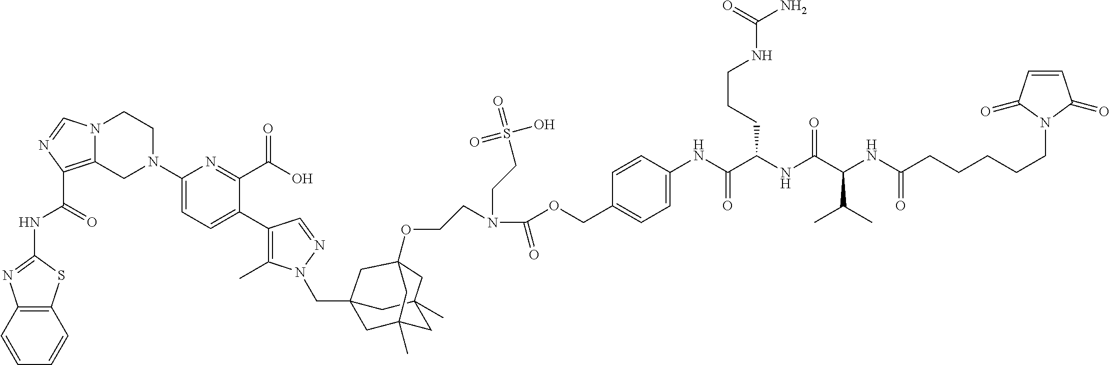 [SMILES: Cc1c(-c2ccc(N3CCn4cnc(C(=O)Nc5nc6ccccc6s5)c4C3)nc2C(=O)O)cnn1CC12CC3(C)CC(C)(C1)CC(OCCN(CCS(=O)(=O)O)C(=O)OCc1ccc(NC(=O)[C@H](CCCNC(N)=O)NC(=O)[C@@H](NC(=O)CCCCCN4C(=O)C=CC4=O)C(C)C)cc1)(C3)C2] |
| 2.110 | RM | 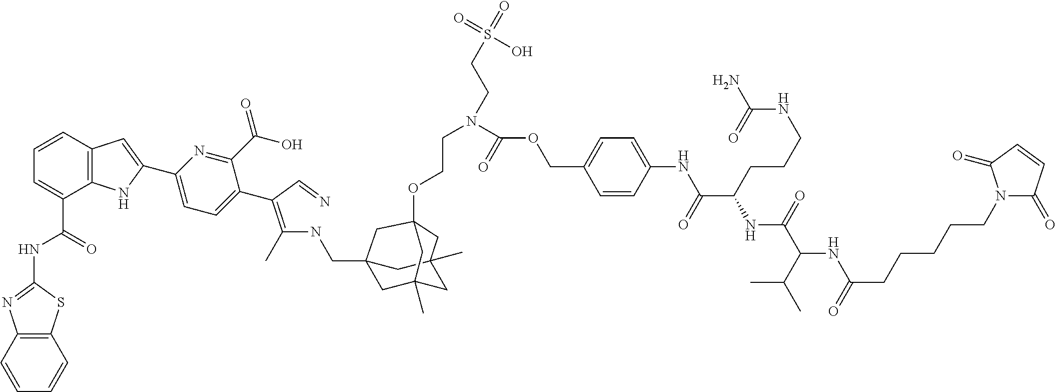 [SMILES: C/C(NCC12CC3(C)CC(C)(C1)CC(OCCN(CCS(=O)(=O)O)C(=O)OCc1ccc(NC(=O)[C@H](CCCNC(N)=O)NC(=O)C(NC(=O)CCCCCN4C(=O)C=CC4=O)C(C)C)cc1)(C3)C2)=C(/C=N)c1ccc(-c2cc3cccc(C(=O)Nc4nc5ccccc5s4)c3[nH]2)nc1C(=O)O] |
| 2.111 | RR | 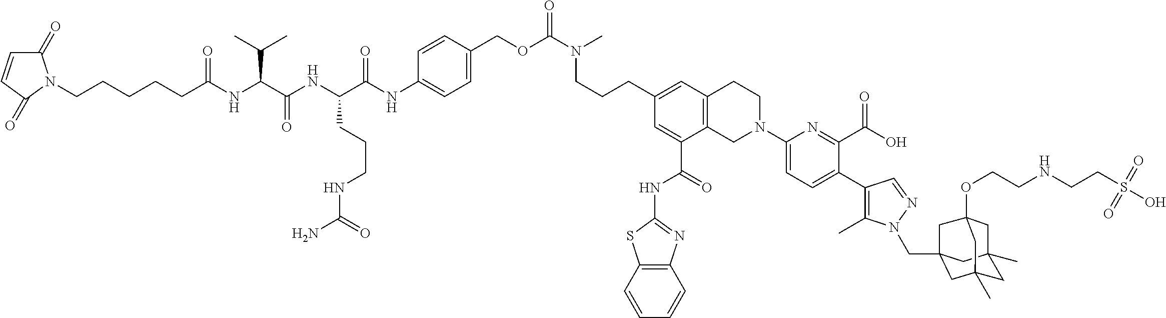 [SMILES: Cc1c(-c2ccc(N3CCc4cc(CCCN(C)C(=O)OCc5ccc(NC(=O)[C@H](CCCNC(N)=O)NC(=O)[C@@H](NC(=O)CCCCCN6C(=O)C=CC6=O)C(C)C)cc5)cc(C(=O)Nc5nc6ccccc6s5)c4C3)nc2C(=O)O)cnn1CC12CC3(C)CC(C)(C1)CC(OCCNCCS(=O)(=O)O)(C3)C2] |
| 2.112 | SJ | 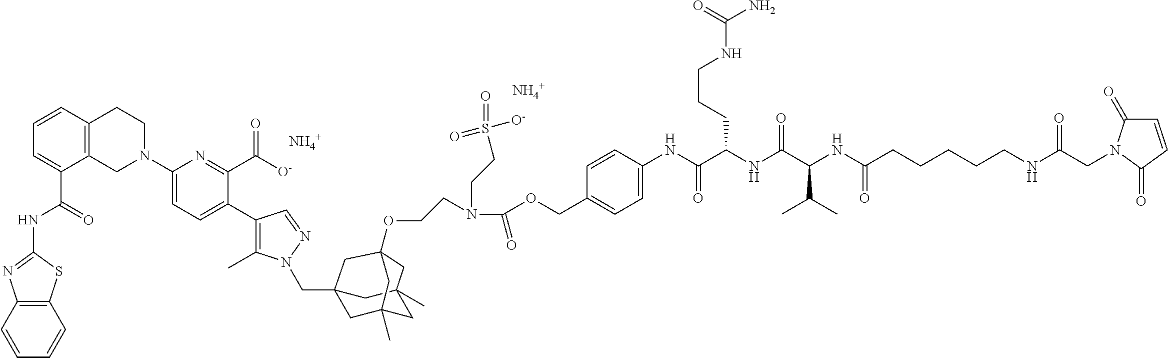 [SMILES: Cc1c(-c2ccc(N3CCc4cccc(C(=O)Nc5nc6ccccc6s5)c4C3)nc2C(=O)[O-])cnn1CC12CC3(C)CC(C)(C1)CC(OCCN(CCS(=O)(=O)[O-])C(=O)OCc1ccc(NC(=O)[C@H](CCCNC(N)=O)NC(=O)[C@@H](NC(=O)CCCCCNC(=O)CN4C(=O)C=CC4=O)C(C)C)cc1)(C3)C2 ; [NH4+] ; [NH4+]] |
| 2.113 | SM | 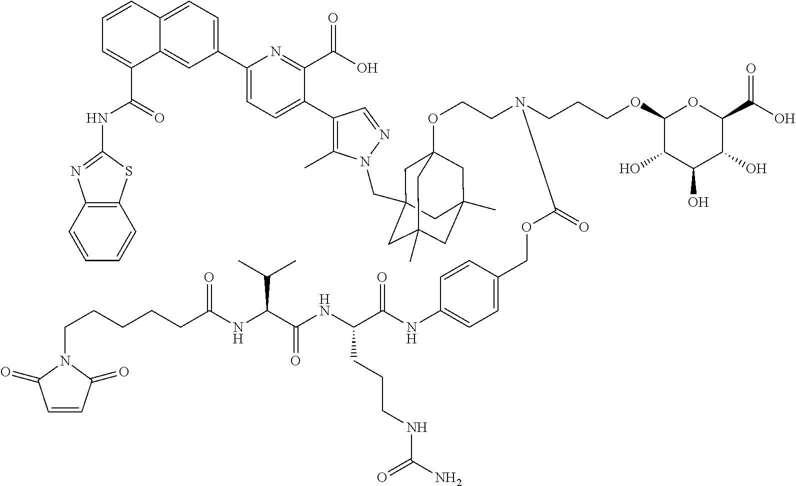 [SMILES: Cc1c(-c2ccc(-c3ccc4cccc(C(=O)Nc5nc6ccccc6s5)c4c3)nc2C(=O)O)cnn1CC12CC3(C)CC(C)(C1)CC(OCCN(CCCO[C@H]1O[C@@H](C(=O)O)[C@H](O)[C@@H](O)[C@@H]1O)C(=O)OCc1ccc(NC(=O)[C@H](CCCNC(N)=O)NC(=O)[C@@H](NC(=O)CCCCCN4C(=O)C=CC4=O)C(C)C)cc1)(C3)C2] |
| 2.114 | SN | 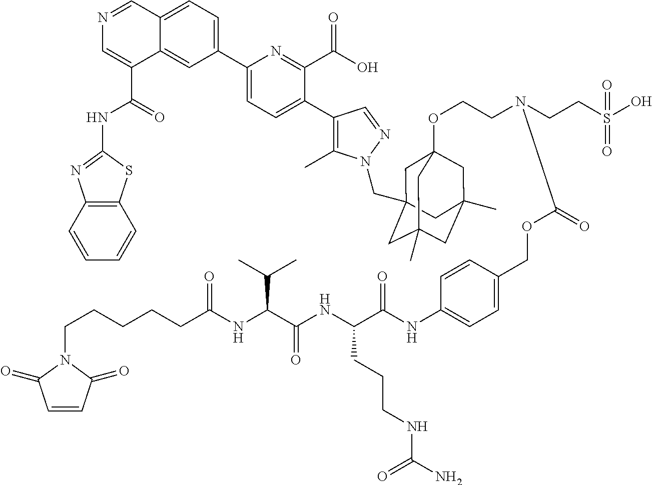 [SMILES: Cc1c(-c2ccc(-c3ccc4cncc(C(=O)Nc5nc6ccccc6s5)c4c3)nc2C(=O)O)cnn1CC12CC3(C)CC(C)(C1)CC(OCCN(CCS(=O)(=O)O)C(=O)OCc1ccc(NC(=O)[C@H](CCCNC(N)=O)NC(=O)[C@@H](NC(=O)CCCCCN4C(=O)C=CC4=O)C(C)C)cc1)(C3)C2] |
| 2.115 | SS | 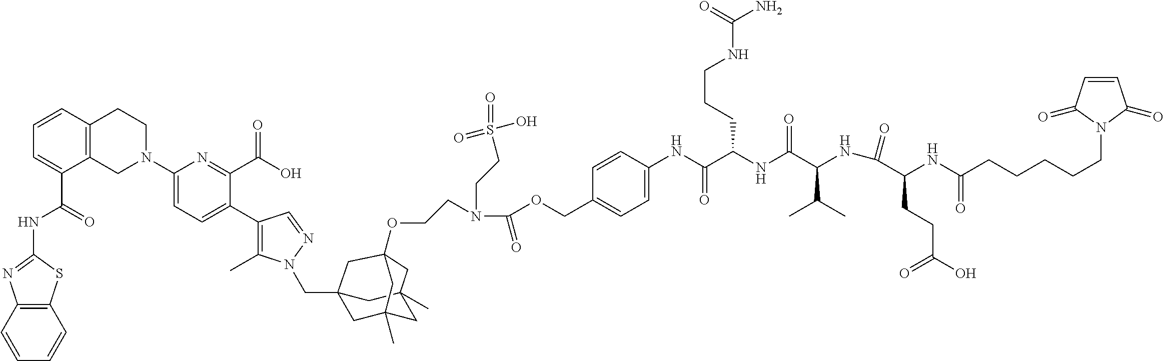 [SMILES: Cc1c(-c2ccc(N3CCc4cccc(C(=O)Nc5nc6ccccc6s5)c4C3)nc2C(=O)O)cnn1CC12CC3(C)CC(C)(C1)CC(OCCN(CCS(=O)(=O)O)C(=O)OCc1ccc(NC(=O)[C@H](CCCNC(N)=O)NC(=O)[C@@H](NC(=O)[C@H](CCC(=O)O)NC(=O)CCCCCN4C(=O)C=CC4=O)C(C)C)cc1)(C3)C2] |
| 2.116 | TA | 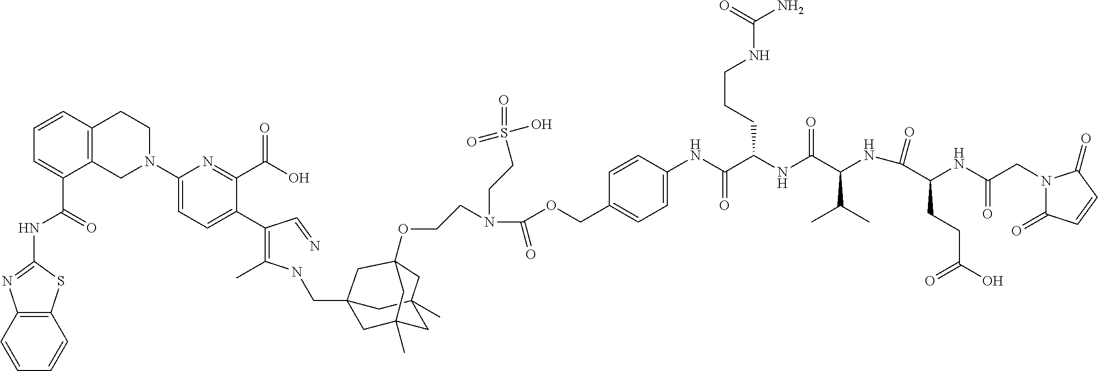 [SMILES: C/C(NCC12CC3(C)CC(C)(C1)CC(OCCN(CCS(=O)(=O)O)C(=O)OCc1ccc(NC(=O)[C@H](CCCNC(N)=O)NC(=O)[C@@H](NC(=O)[C@H](CCC(=O)O)NC(=O)CN4C(=O)C=CC4=O)C(C)C)cc1)(C3)C2)=C(/C=N)c1ccc(N2CCc3cccc(C(=O)Nc4nc5ccccc5s4)c3C2)nc1C(=O)O] |
| 2.117 | TW | 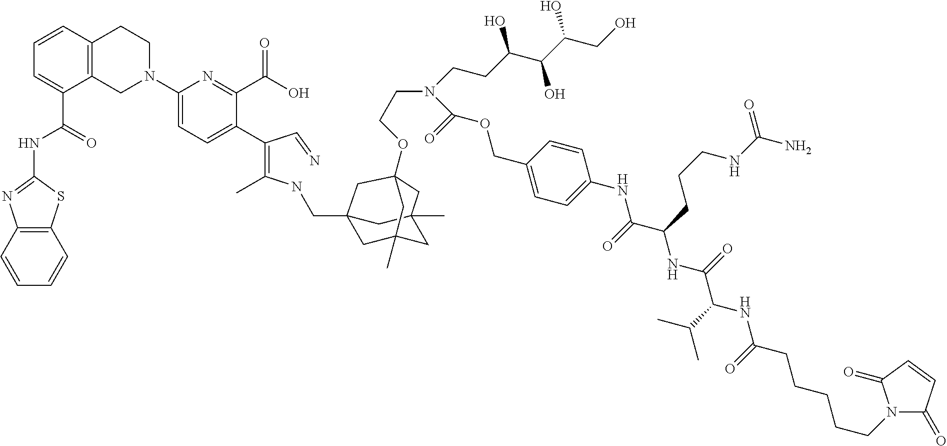 [SMILES: C/C(NCC12CC3(C)CC(C)(C1)CC(OCCN(CC[C@@H](O)[C@H](O)[C@H](O)CO)C(=O)OCc1ccc(NC(=O)[C@@H](CCCNC(N)=O)NC(=O)[C@H](NC(=O)CCCCCN4C(=O)C=CC4=O)C(C)C)cc1)(C3)C2)=C(/C=N)c1ccc(N2CCc3cccc(C(=O)Nc4nc5ccccc5s4)c3C2)nc1C(=O)O] |
| 2.118 | ST | 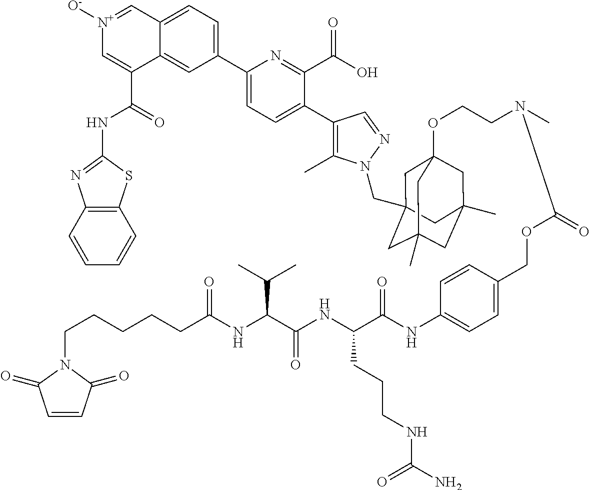 [SMILES: Cc1c(-c2ccc(-c3ccc4c[n+]([O-])cc(C(=O)Nc5nc6ccccc6s5)c4c3)nc2C(=O)O)cnn1CC12CC3(C)CC(C)(C1)CC(OCCN(C)C(=O)OCc1ccc(NC(=O)[C@H](CCCNC(N)=O)NC(=O)[C@@H](NC(=O)CCCCCN4C(=O)C=CC4=O)C(C)C)cc1)(C3)C2] |
| 2.119 | ZL | 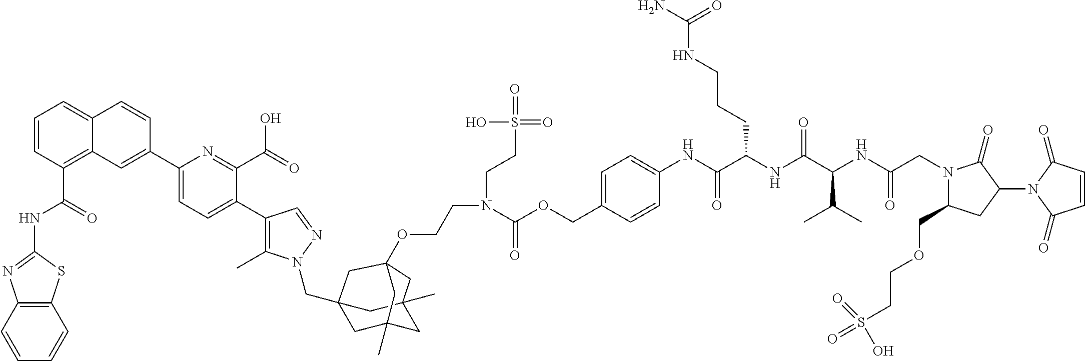 [SMILES: Cc1c(-c2ccc(-c3ccc4cccc(C(=O)Nc5nc6ccccc6s5)c4c3)nc2C(=O)O)cnn1CC12CC3(C)CC(C)(C1)CC(OCCN(CCS(=O)(=O)O)C(=O)OCc1ccc(NC(=O)[C@H](CCCNC(N)=O)NC(=O)[C@@H](NC(=O)CN4C(=O)C(N5C(=O)C=CC5=O)C[C@H]4COCCS(=O)(=O)O)C(C)C)cc1)(C3)C2] |
| 2.120 | SX | 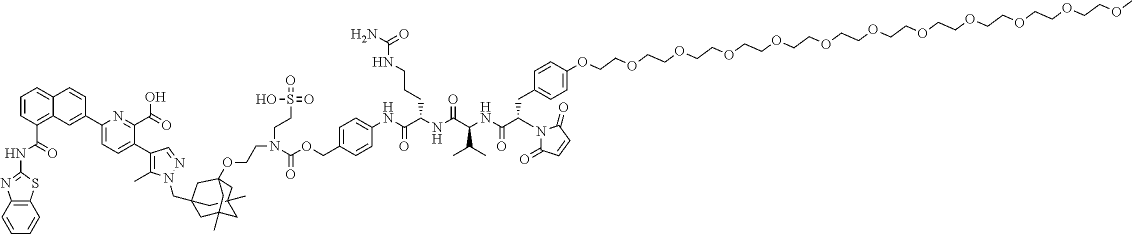 [SMILES: COCCOCCOCCOCCOCCOCCOCCOCCOCCOCCOCCOc1ccc(C[C@@H](C(=O)N[C@H](C(=O)N[C@@H](CCCNC(N)=O)C(=O)Nc2ccc(COC(=O)N(CCOC34CC5(C)CC(C)(CC(Cn6ncc(-c7ccc(-c8ccc9cccc(C(=O)Nc%10nc%11ccccc%11s%10)c9c8)nc7C(=O)O)c6C)(C5)C3)C4)CCS(=O)(=O)O)cc2)C(C)C)N2C(=O)C=CC2=O)cc1] |
| 2.121 | SW | 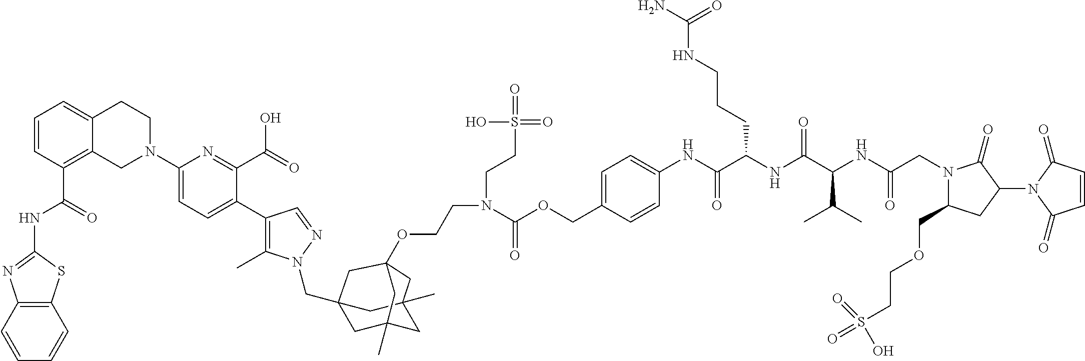 [SMILES: Cc1c(-c2ccc(N3CCc4cccc(C(=O)Nc5nc6ccccc6s5)c4C3)nc2C(=O)O)cnn1CC12CC3(C)CC(C)(C1)CC(OCCN(CCS(=O)(=O)O)C(=O)OCc1ccc(NC(=O)[C@H](CCCNC(N)=O)NC(=O)[C@@H](NC(=O)CN4C(=O)C(N5C(=O)C=CC5=O)C[C@H]4COCCS(=O)(=O)O)C(C)C)cc1)(C3)C2] |
| 2.122 | TV | 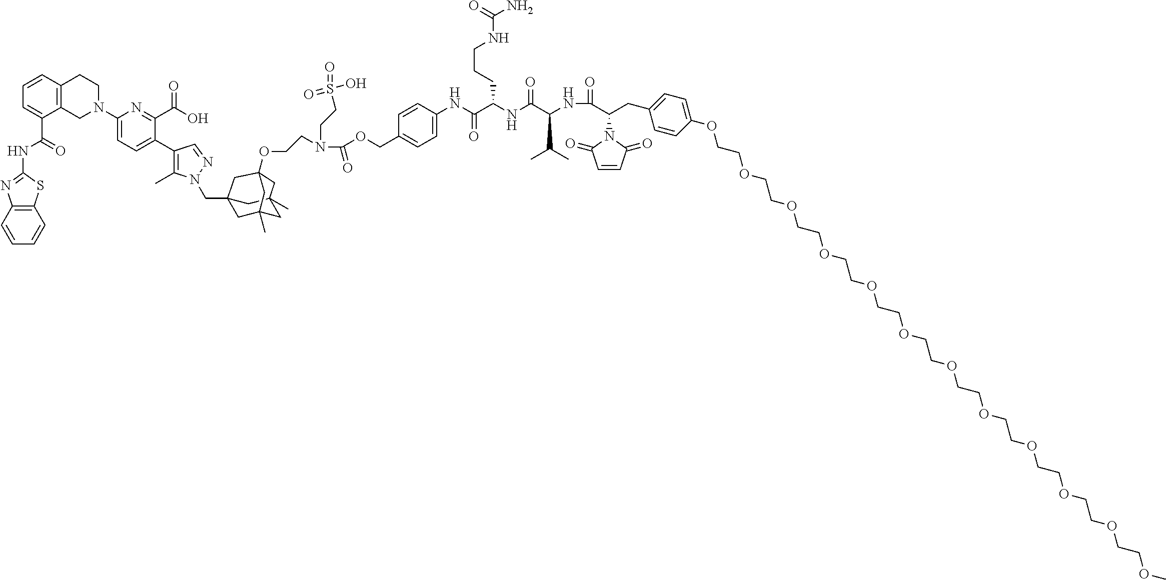 [SMILES: COCCOCCOCCOCCOCCOCCOCCOCCOCCOCCOCCOc1ccc(C[C@@H](C(=O)N[C@H](C(=O)N[C@@H](CCCNC(N)=O)C(=O)Nc2ccc(COC(=O)N(CCOC34CC5(C)CC(C)(CC(Cn6ncc(-c7ccc(N8CCc9cccc(C(=O)Nc%10nc%11ccccc%11s%10)c9C8)nc7C(=O)O)c6C)(C5)C3)C4)CCS(=O)(=O)O)cc2)C(C)C)N2C(=O)C=CC2=O)cc1] |
| 2.123 | SZ | 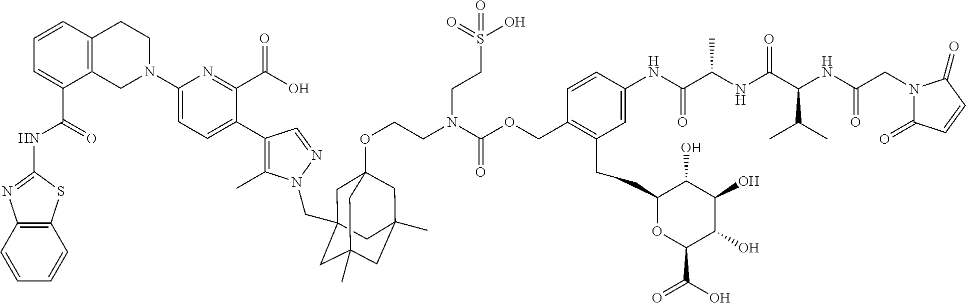 [SMILES: Cc1c(-c2ccc(N3CCc4cccc(C(=O)Nc5nc6ccccc6s5)c4C3)nc2C(=O)O)cnn1CC12CC3(C)CC(C)(C1)CC(OCCN(CCS(=O)(=O)O)C(=O)OCc1ccc(NC(=O)[C@H](C)NC(=O)[C@@H](NC(=O)CN4C(=O)C=CC4=O)C(C)C)cc1CC[C@@H]1O[C@H](C(=O)O)[C@@H](O)[C@H](O)[C@H]1O)(C3)C2] |
| 2.124 | ZM | 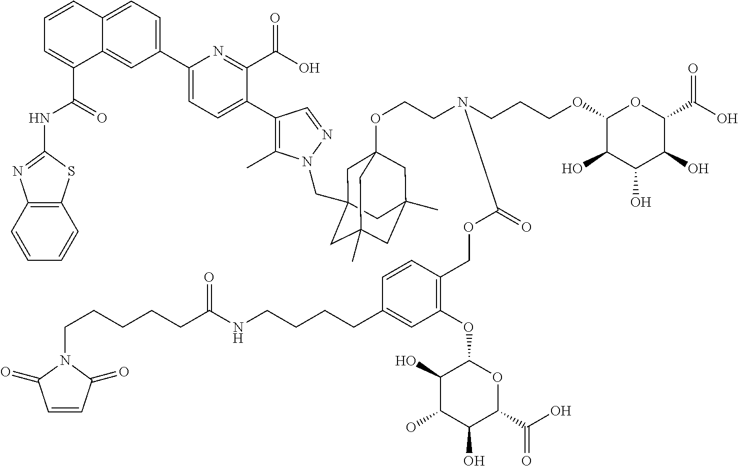 [SMILES: Cc1c(-c2ccc(-c3ccc4cccc(C(=O)Nc5nc6ccccc6s5)c4c3)nc2C(=O)O)cnn1CC12CC3(C)CC(C)(C1)CC(OCCN(CCCO[C@@H]1O[C@H](C(=O)O)[C@@H](O)[C@H](O)[C@H]1O)C(=O)OCc1ccc(CCCCNC(=O)CCCCCN4C(=O)C=CC4=O)cc1O[C@@H]1O[C@H](C(=O)O)[C@@H](O)[C@H](O)[C@H]1O)(C3)C2] |
| 2.125 | SV | 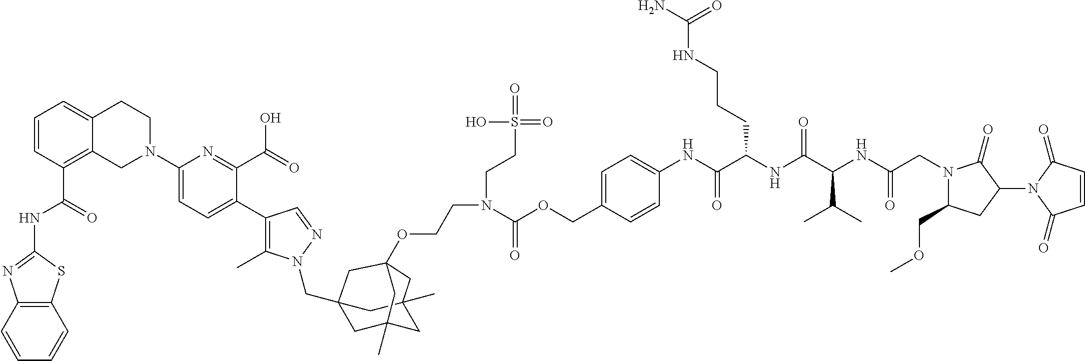 [SMILES: COC[C@@H]1CC(N2C(=O)C=CC2=O)C(=O)N1CC(=O)N[C@H](C(=O)N[C@@H](CCCNC(N)=O)C(=O)Nc1ccc(COC(=O)N(CCOC23CC4(C)CC(C)(CC(Cn5ncc(-c6ccc(N7CCc8cccc(C(=O)Nc9nc%10ccccc%10s9)c8C7)nc6C(=O)O)c5C)(C4)C2)C3)CCS(=O)(=O)O)cc1)C(C)C] |
| 2.126 | SY | 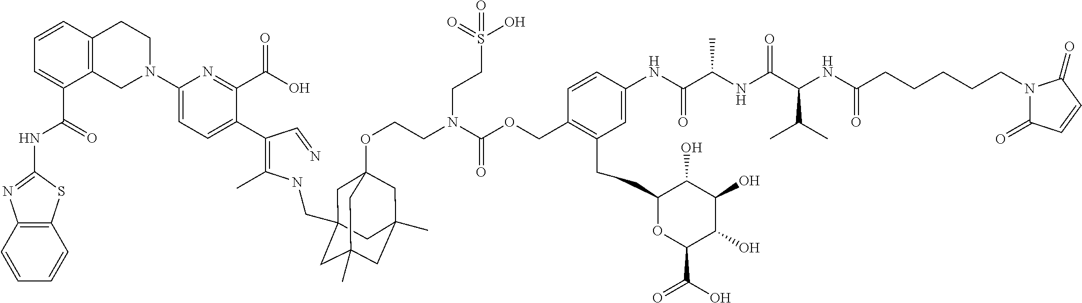 [SMILES: C/C(NCC12CC3(C)CC(C)(C1)CC(OCCN(CCS(=O)(=O)O)C(=O)OCc1ccc(NC(=O)[C@H](C)NC(=O)[C@@H](NC(=O)CCCCCN4C(=O)C=CC4=O)C(C)C)cc1CC[C@@H]1O[C@H](C(=O)O)[C@@H](O)[C@H](O)[C@H]1O)(C3)C2)=C(/C=N)c1ccc(N2CCc3cccc(C(=O)Nc4nc5ccccc5s4)c3C2)nc1C(=O)O] |
| 2.127 | TK | 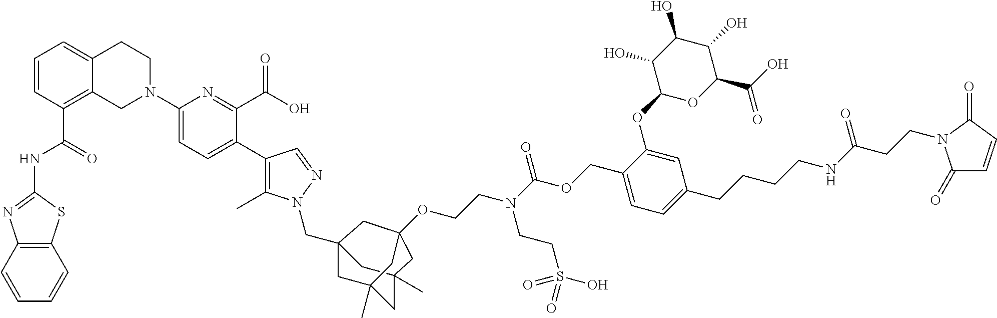 [SMILES: Cc1c(-c2ccc(N3CCc4cccc(C(=O)Nc5nc6ccccc6s5)c4C3)nc2C(=O)O)cnn1CC12CC3(C)CC(C)(C1)CC(OCCN(CCS(=O)(=O)O)C(=O)OCc1ccc(CCCCNC(=O)CCN4C(=O)C=CC4=O)cc1O[C@@H]1O[C@H](C(=O)O)[C@@H](O)[C@H](O)[C@H]1O)(C3)C2] |
| 2.128 | TR | 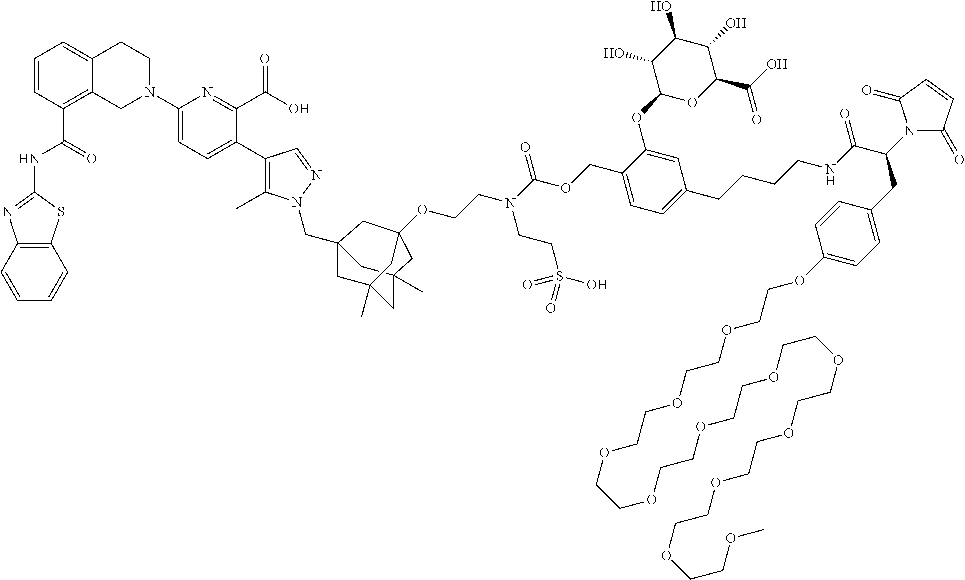 [SMILES: COCCOCCOCCOCCOCCOCCOCCOCCOCCOCCOCCOc1ccc(C[C@@H](C(=O)NCCCCc2ccc(COC(=O)N(CCOC34CC5(C)CC(C)(CC(Cn6ncc(-c7ccc(N8CCc9cccc(C(=O)Nc%10nc%11ccccc%11s%10)c9C8)nc7C(=O)O)c6C)(C5)C3)C4)CCS(=O)(=O)O)c(O[C@@H]3O[C@H](C(=O)O)[C@@H](O)[C@H](O)[C@H]3O)c2)N2C(=O)C=CC2=O)cc1] |
| 2.129 | TY | 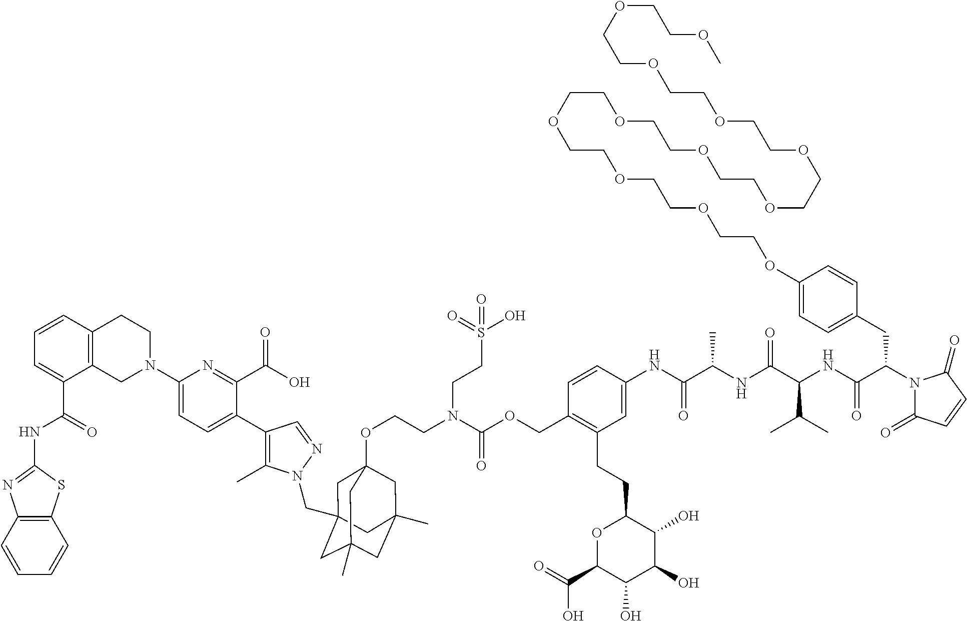 [SMILES: COCCOCCOCCOCCOCCOCCOCCOCCOCCOCCOCCOc1ccc(C[C@@H](C(=O)N[C@H](C(=O)N[C@@H](C)C(=O)Nc2ccc(COC(=O)N(CCOC34CC5(C)CC(C)(CC(Cn6ncc(-c7ccc(N8CCc9cccc(C(=O)Nc%10nc%11ccccc%11s%10)c9C8)nc7C(=O)O)c6C)(C5)C3)C4)CCS(=O)(=O)O)c(CC[C@@H]3O[C@H](C(=O)O)[C@@H](O)[C@H](O)[C@H]3O)c2)C(C)C)N2C(=O)C=CC2=O)cc1] |
| 2.130 | TX | 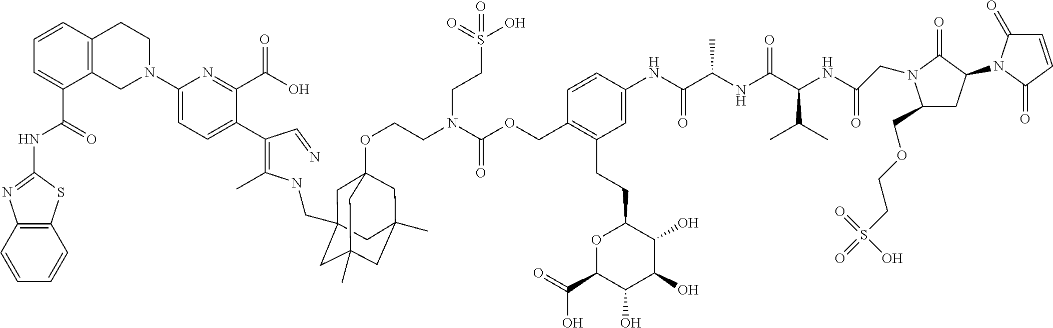 [SMILES: C/C(NCC12CC3(C)CC(C)(C1)CC(OCCN(CCS(=O)(=O)O)C(=O)OCc1ccc(NC(=O)[C@H](C)NC(=O)[C@@H](NC(=O)CN4C(=O)[C@@H](N5C(=O)C=CC5=O)C[C@H]4COCCS(=O)(=O)O)C(C)C)cc1CC[C@@H]1O[C@H](C(=O)O)[C@@H](O)[C@H](O)[C@H]1O)(C3)C2)=C(/C=N)c1ccc(N2CCc3cccc(C(=O)Nc4nc5ccccc5s4)c3C2)nc1C(=O)O] |
| 2.131 | TZ | 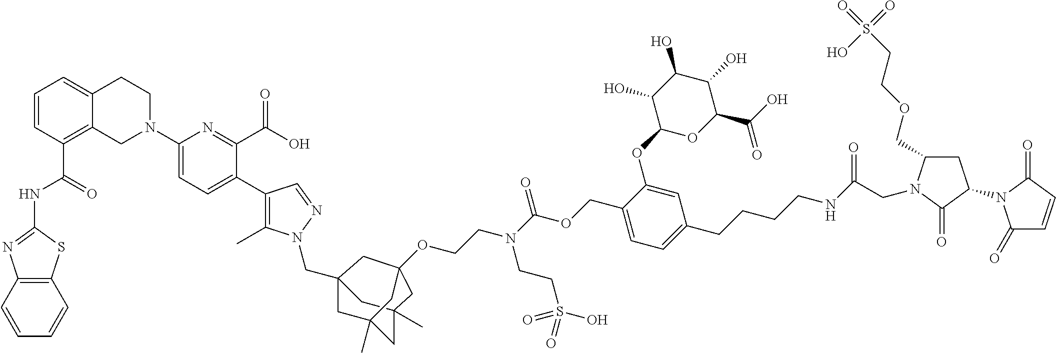 [SMILES: Cc1c(-c2ccc(N3CCc4cccc(C(=O)Nc5nc6ccccc6s5)c4C3)nc2C(=O)O)cnn1CC12CC3(C)CC(C)(C1)CC(OCCN(CCS(=O)(=O)O)C(=O)OCc1ccc(CCCCNC(=O)CN4C(=O)[C@@H](N5C(=O)C=CC5=O)C[C@H]4COCCS(=O)(=O)O)cc1O[C@@H]1O[C@H](C(=O)O)[C@@H](O)[C@H](O)[C@H]1O)(C3)C2] |
| 2.132 | UA | 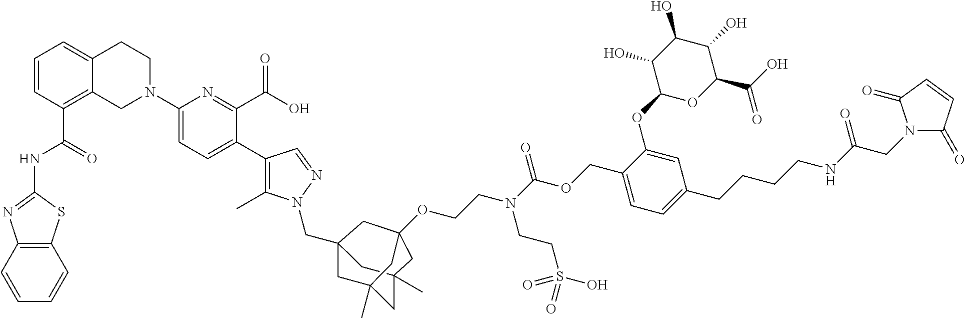 [SMILES: Cc1c(-c2ccc(N3CCc4cccc(C(=O)Nc5nc6ccccc6s5)c4C3)nc2C(=O)O)cnn1CC12CC3(C)CC(C)(C1)CC(OCCN(CCS(=O)(=O)O)C(=O)OCc1ccc(CCCCNC(=O)CN4C(=O)C=CC4=O)cc1O[C@@H]1O[C@H](C(=O)O)[C@@H](O)[C@H](O)[C@H]1O)(C3)C2] |
| 2.133 | UJ | 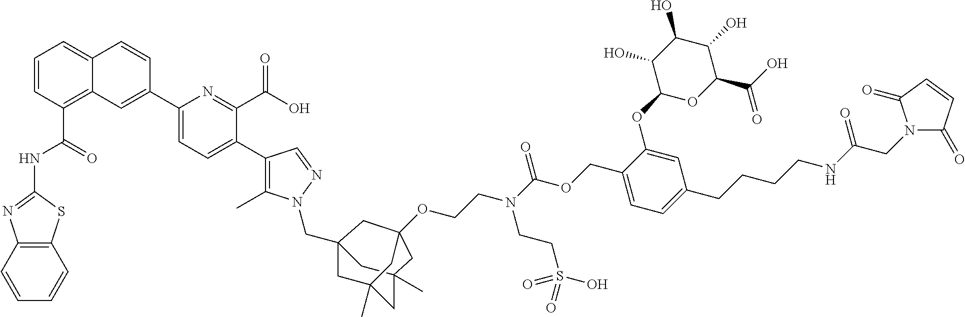 [SMILES: Cc1c(-c2ccc(-c3ccc4cccc(C(=O)Nc5nc6ccccc6s5)c4c3)nc2C(=O)O)cnn1CC12CC3(C)CC(C)(C1)CC(OCCN(CCS(=O)(=O)O)C(=O)OCc1ccc(CCCCNC(=O)CN4C(=O)C=CC4=O)cc1O[C@@H]1O[C@H](C(=O)O)[C@@H](O)[C@H](O)[C@H]1O)(C3)C2] |
| 2.134 | UK | 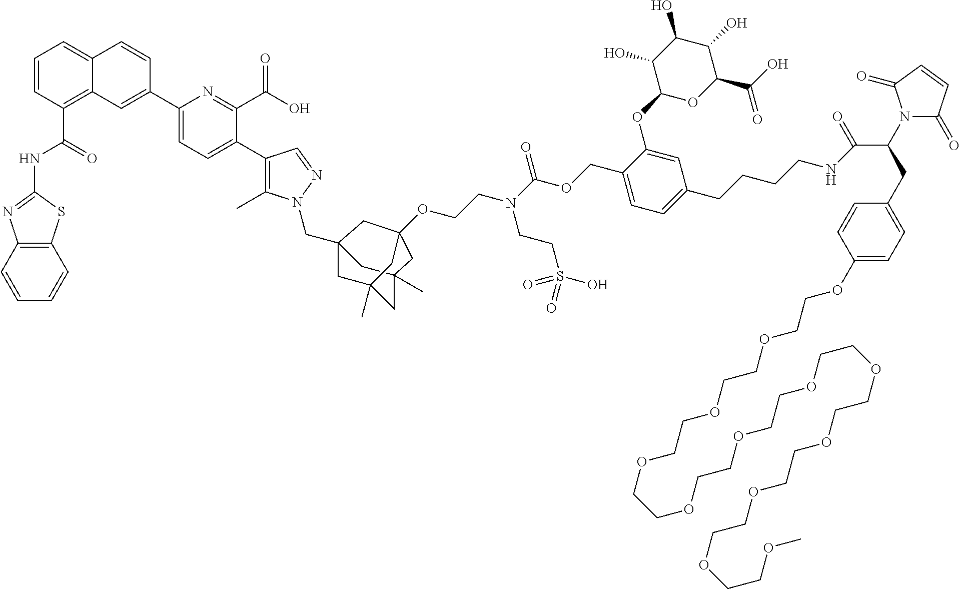 [SMILES: COCCOCCOCCOCCOCCOCCOCCOCCOCCOCCOCCOc1ccc(C[C@@H](C(=O)NCCCCc2ccc(COC(=O)N(CCOC34CC5(C)CC(C)(CC(Cn6ncc(-c7ccc(-c8ccc9cccc(C(=O)Nc%10nc%11ccccc%11s%10)c9c8)nc7C(=O)O)c6C)(C5)C3)C4)CCS(=O)(=O)O)c(O[C@@H]3O[C@H](C(=O)O)[C@@H](O)[C@H](O)[C@H]3O)c2)N2C(=O)C=CC2=O)cc1] |
| 2.135 | UU | 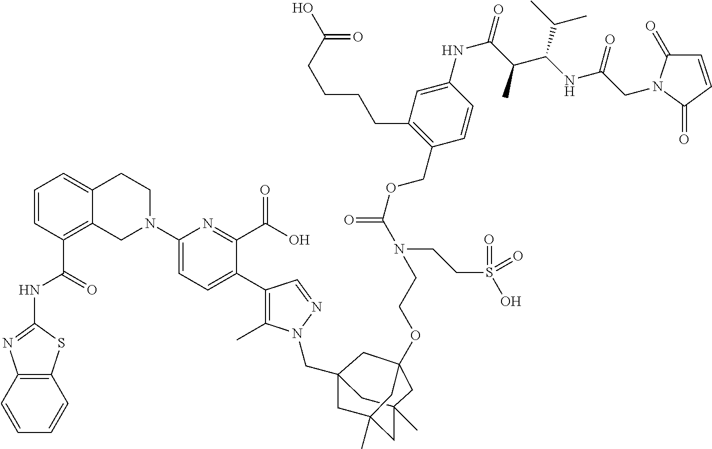 [SMILES: Cc1c(-c2ccc(N3CCc4cccc(C(=O)Nc5nc6ccccc6s5)c4C3)nc2C(=O)O)cnn1CC12CC3(C)CC(C)(C1)CC(OCCN(CCS(=O)(=O)O)C(=O)OCc1ccc(NC(=O)[C@H](C)[C@@H](NC(=O)CN4C(=O)C=CC4=O)C(C)C)cc1CCCCC(=O)O)(C3)C2] |
| 2.136 | UV | 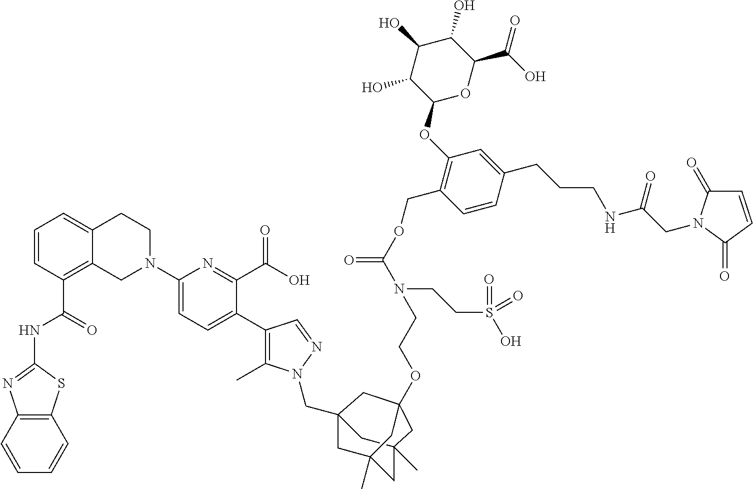 [SMILES: Cc1c(-c2ccc(N3CCc4cccc(C(=O)Nc5nc6ccccc6s5)c4C3)nc2C(=O)O)cnn1CC12CC3(C)CC(C)(C1)CC(OCCN(CCS(=O)(=O)O)C(=O)OCc1ccc(CCCNC(=O)CN4C(=O)C=CC4=O)cc1O[C@@H]1O[C@H](C(=O)O)[C@@H](O)[C@H](O)[C@H]1O)(C3)C2] |
| 2.137 | UZ | 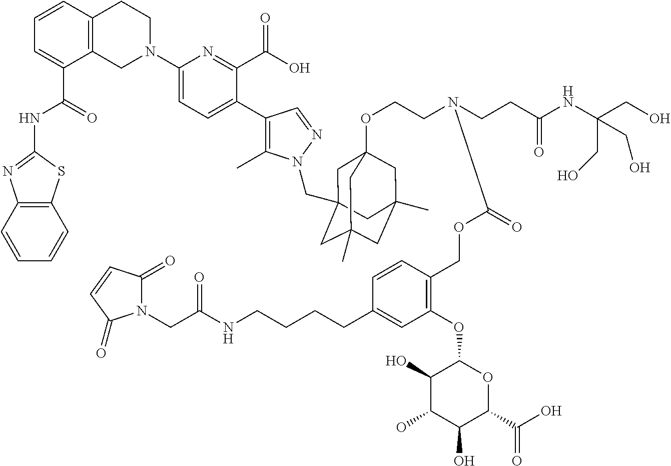 [SMILES: Cc1c(-c2ccc(N3CCc4cccc(C(=O)Nc5nc6ccccc6s5)c4C3)nc2C(=O)O)cnn1CC12CC3(C)CC(C)(C1)CC(OCCN(CCC(=O)NC(CO)(CO)CO)C(=O)OCc1ccc(CCCCNC(=O)CN4C(=O)C=CC4=O)cc1O[C@@H]1O[C@H](C(=O)O)[C@@H](O)[C@H](O)[C@H]1O)(C3)C2] |
| 2.138 | VB | 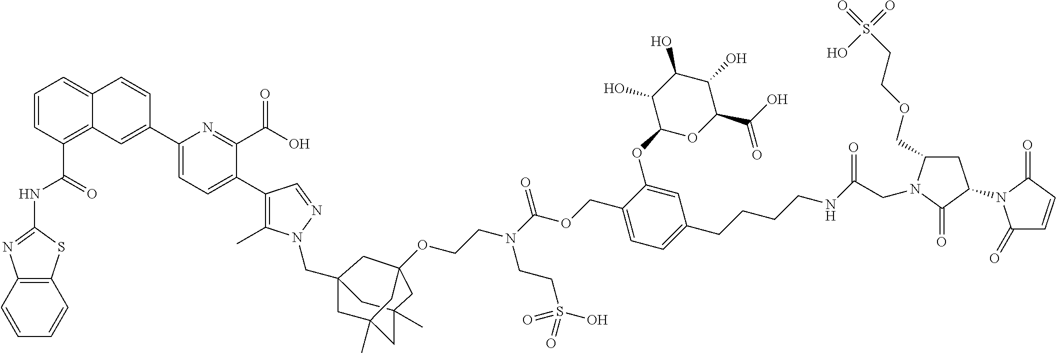 [SMILES: Cc1c(-c2ccc(-c3ccc4cccc(C(=O)Nc5nc6ccccc6s5)c4c3)nc2C(=O)O)cnn1CC12CC3(C)CC(C)(C1)CC(OCCN(CCS(=O)(=O)O)C(=O)OCc1ccc(CCCCNC(=O)CN4C(=O)[C@@H](N5C(=O)C=CC5=O)C[C@H]4COCCS(=O)(=O)O)cc1O[C@@H]1O[C@H](C(=O)O)[C@@H](O)[C@H](O)[C@H]1O)(C3)C2] |
| 2.139 | VC | 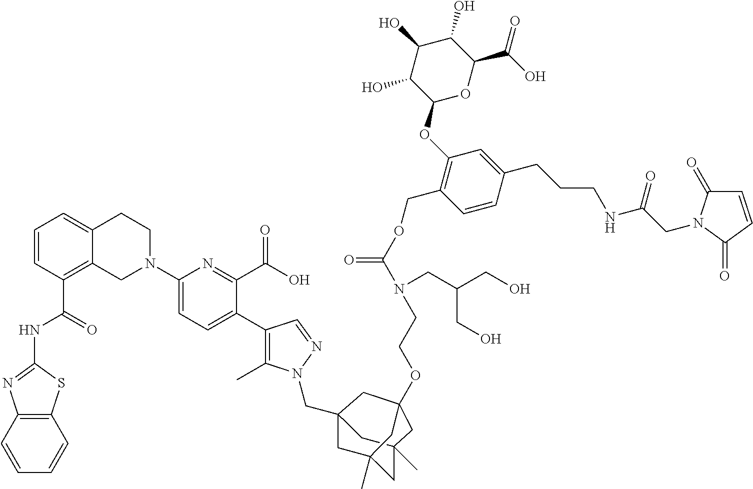 [SMILES: Cc1c(-c2ccc(N3CCc4cccc(C(=O)Nc5nc6ccccc6s5)c4C3)nc2C(=O)O)cnn1CC12CC3(C)CC(C)(C1)CC(OCCN(CC(CO)CO)C(=O)OCc1ccc(CCCNC(=O)CN4C(=O)C=CC4=O)cc1O[C@@H]1O[C@H](C(=O)O)[C@@H](O)[C@H](O)[C@H]1O)(C3)C2] |
| 2.140 | VS | 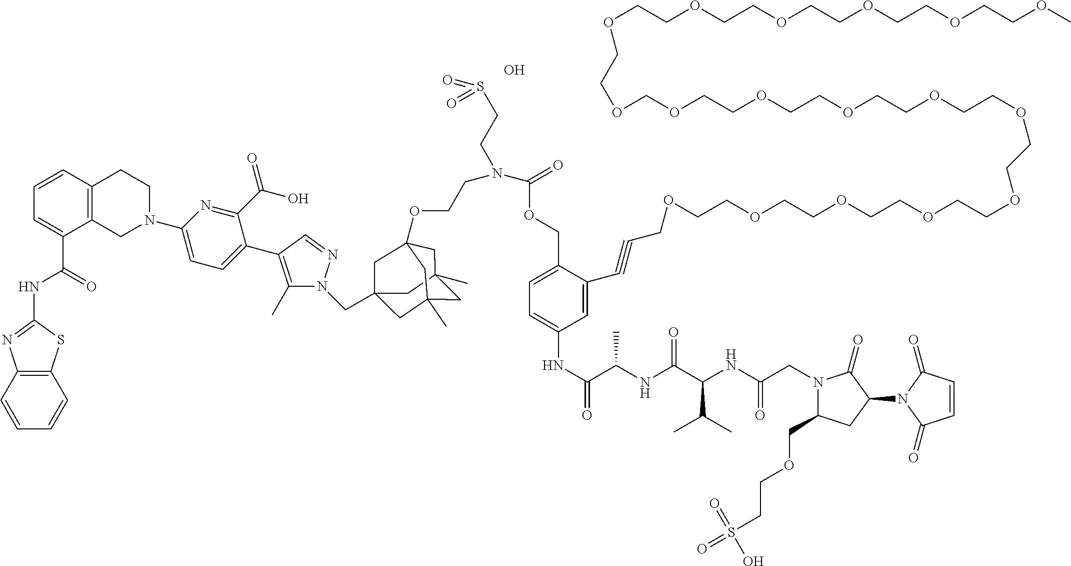 |
| 2.141 | VT | 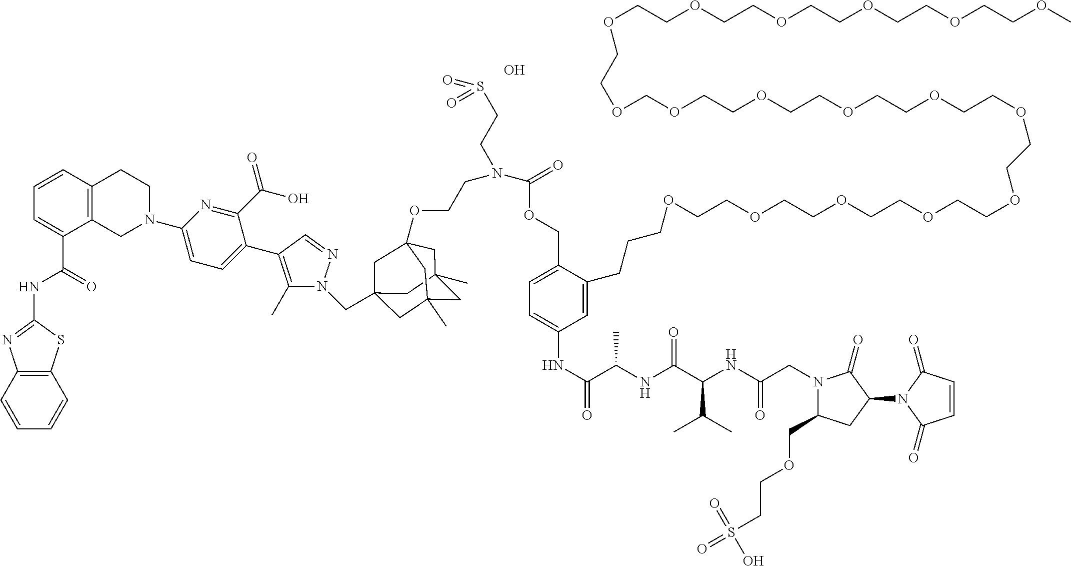 |
| 2.142 | VY | 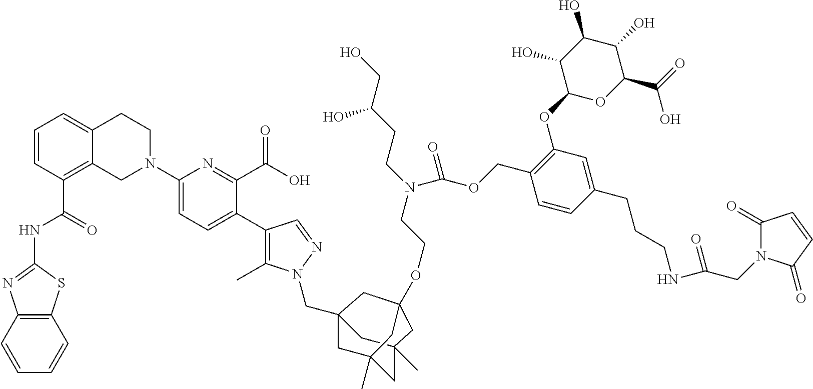 [SMILES: Cc1c(-c2ccc(N3CCc4cccc(C(=O)Nc5nc6ccccc6s5)c4C3)nc2C(=O)O)cnn1CC12CC3(C)CC(C)(C1)CC(OCCN(CC[C@H](O)CO)C(=O)OCc1ccc(CCCNC(=O)CN4C(=O)C=CC4=O)cc1O[C@@H]1O[C@H](C(=O)O)[C@@H](O)[C@H](O)[C@H]1O)(C3)C2] |
| 2.143 | WI | 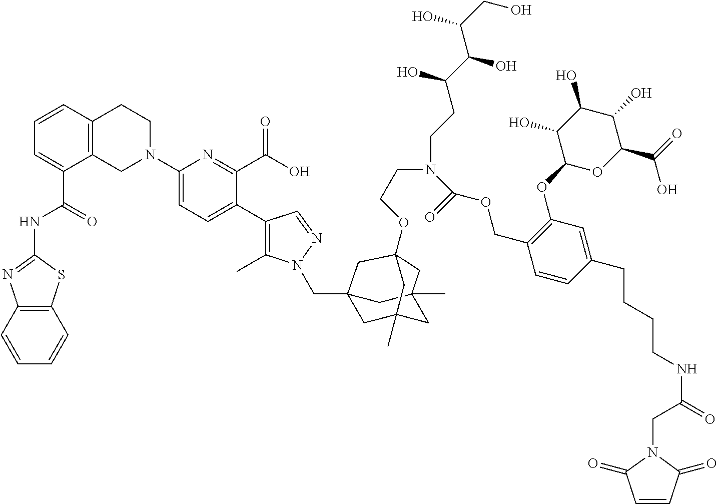 [SMILES: Cc1c(-c2ccc(N3CCc4cccc(C(=O)Nc5nc6ccccc6s5)c4C3)nc2C(=O)O)cnn1CC12CC3(C)CC(C)(C1)CC(OCCN(CC[C@@H](O)[C@H](O)[C@H](O)CO)C(=O)OCc1ccc(CCCCNC(=O)CN4C(=O)C=CC4=O)cc1O[C@@H]1O[C@H](C(=O)O)[C@@H](O)[C@H](O)[C@H]1O)(C3)C2] |
| 2.144 | WK | 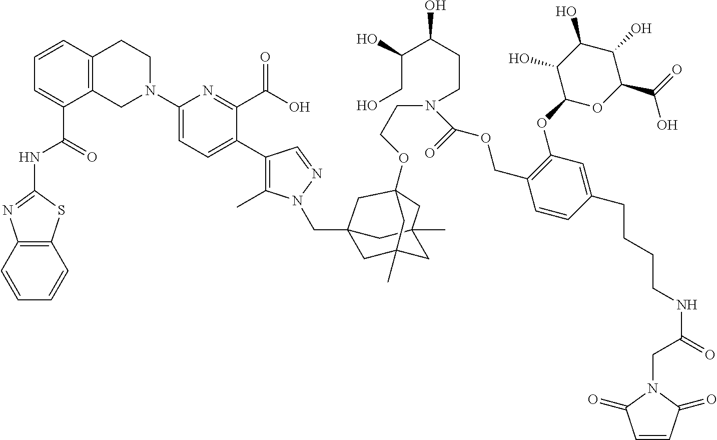 [SMILES: Cc1c(-c2ccc(N3CCc4cccc(C(=O)Nc5nc6ccccc6s5)c4C3)nc2C(=O)O)cnn1CC12CC3(C)CC(C)(C1)CC(OCCN(CC[C@H](O)[C@H](O)CO)C(=O)OCc1ccc(CCCCNC(=O)CN4C(=O)C=CC4=O)cc1O[C@@H]1O[C@H](C(=O)O)[C@@H](O)[C@H](O)[C@H]1O)(C3)C2] |
| 2.145 | WP | 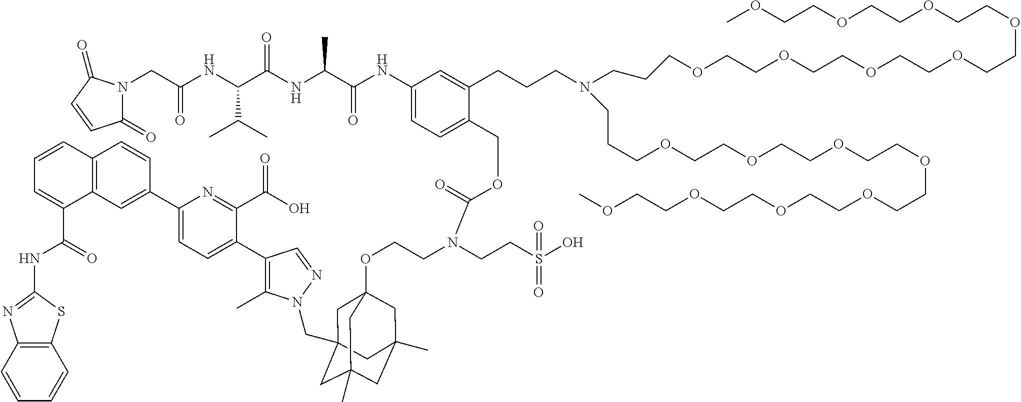 [SMILES: COCCOCCOCCOCCOCCOCCOCCOCCCN(CCCOCCOCCOCCOCCOCCOCCOCCOC)CCCc1cc(NC(=O)[C@H](C)NC(=O)[C@@H](NC(=O)CN2C(=O)C=CC2=O)C(C)C)ccc1COC(=O)N(CCOC12CC3(C)CC(C)(CC(Cn4ncc(-c5ccc(-c6ccc7cccc(C(=O)Nc8nc9ccccc9s8)c7c6)nc5C(=O)O)c4C)(C3)C1)C2)CCS(=O)(=O)O] |
| 2.146 | XD | 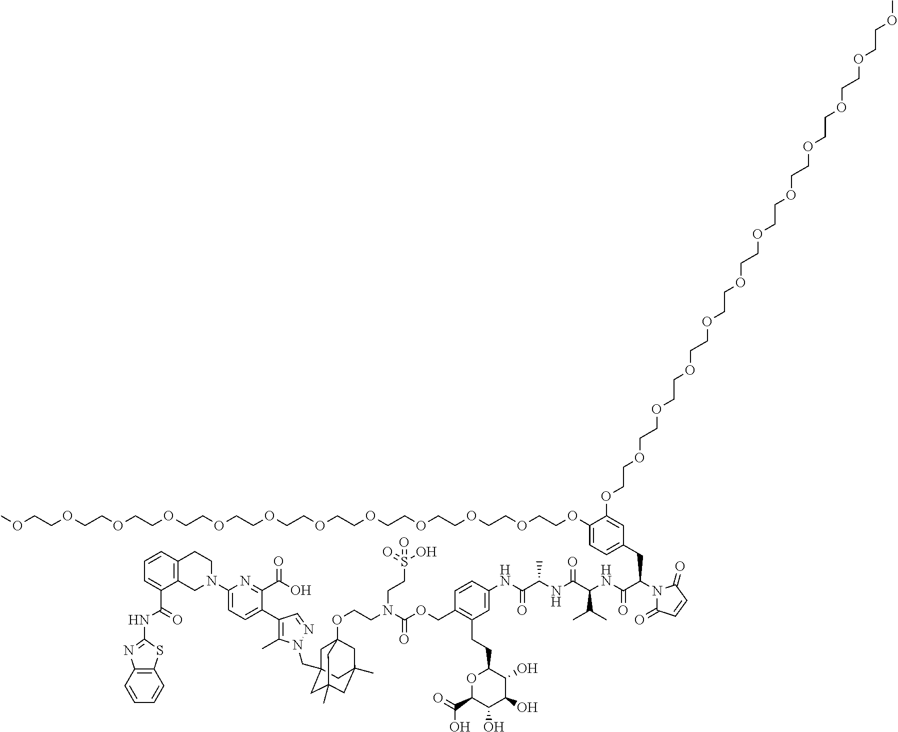 |
| 2.147 | XK | 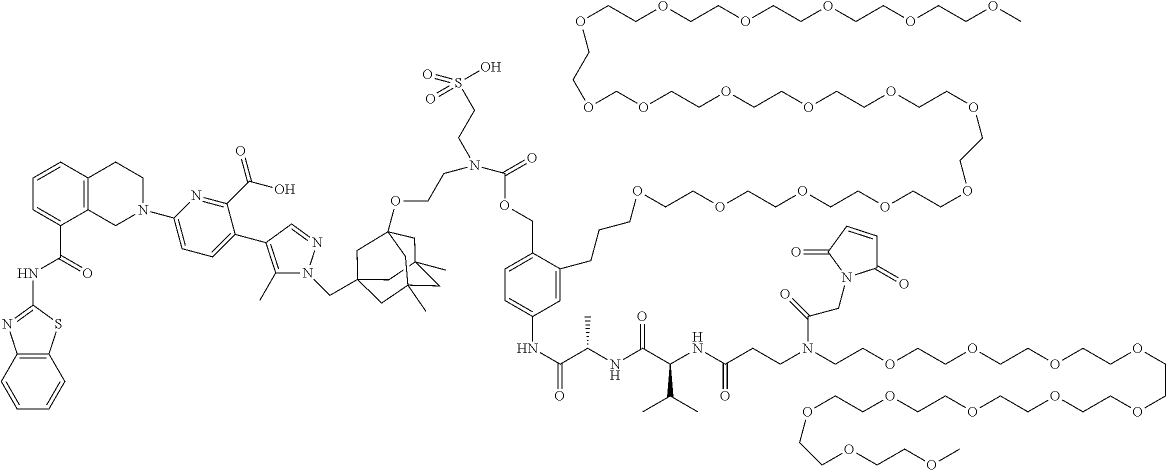 [SMILES: COCCOCCOCCOCCOCCOCCOCCOCCOCCOCCOCCN(CCC(=O)N[C@H](C(=O)N[C@@H](C)C(=O)Nc1ccc(COC(=O)N(CCOC23CC4(C)CC(C)(CC(Cn5ncc(-c6ccc(N7CCc8cccc(C(=O)Nc9nc%10ccccc%10s9)c8C7)nc6C(=O)O)c5C)(C4)C2)C3)CCS(=O)(=O)O)c(CCCOCCOCCOCCOCCOCCOCCOCCOCCOCCOCOCCOCCOCCOCCOCCOCCOC)c1)C(C)C)C(=O)CN1C(=O)C=CC1=O] |
| 2.148 | XL | 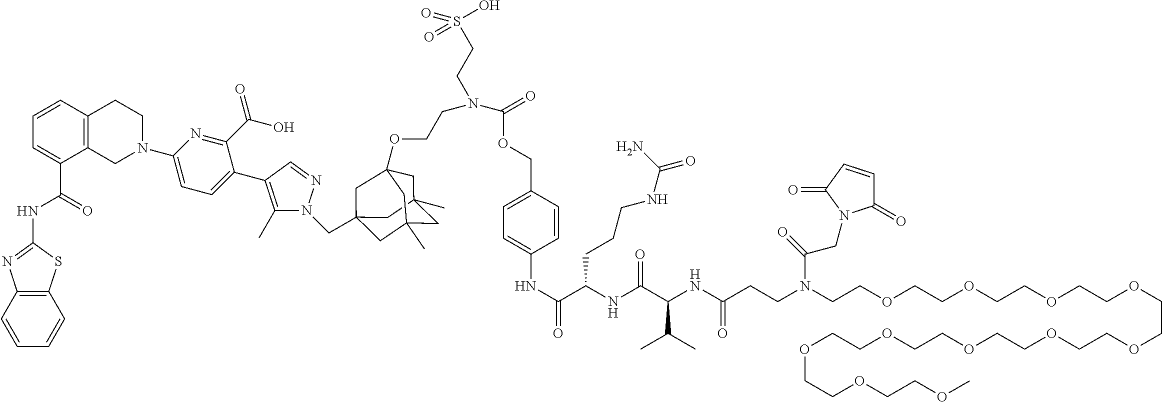 [SMILES: COCCOCCOCCOCCOCCOCCOCCOCCOCCOCCOCCN(CCC(=O)N[C@H](C(=O)N[C@@H](CCCNC(N)=O)C(=O)Nc1ccc(COC(=O)N(CCOC23CC4(C)CC(C)(CC(Cn5ncc(-c6ccc(N7CCc8cccc(C(=O)Nc9nc%10ccccc%10s9)c8C7)nc6C(=O)O)c5C)(C4)C2)C3)CCS(=O)(=O)O)cc1)C(C)C)C(=O)CN1C(=O)C=CC1=O] |
| 2.149 | YJ | 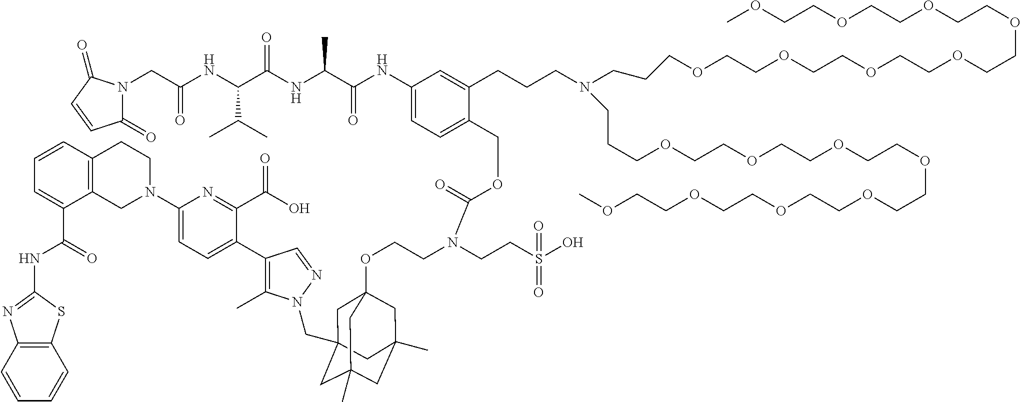 [SMILES: COCCOCCOCCOCCOCCOCCOCCOCCCN(CCCOCCOCCOCCOCCOCCOCCOCCOC)CCCc1cc(NC(=O)[C@H](C)NC(=O)[C@@H](NC(=O)CN2C(=O)C=CC2=O)C(C)C)ccc1COC(=O)N(CCOC12CC3(C)CC(C)(CC(Cn4ncc(-c5ccc(N6CCc7cccc(C(=O)Nc8nc9ccccc9s8)c7C6)nc5C(=O)O)c4C)(C3)C1)C2)CCS(=O)(=O)O] |
| 2.150 | YQ | 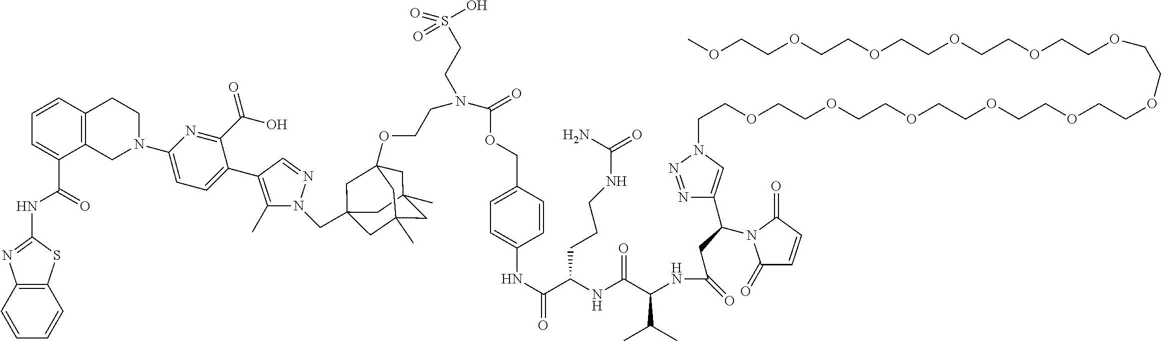 [SMILES: COCCOCCOCCOCCOCCOCCOCCOCCOCCOCCOCCOCCn1cc([C@H](CC(=O)N[C@H](C(=O)N[C@@H](CCCNC(N)=O)C(=O)Nc2ccc(COC(=O)N(CCOC34CC5(C)CC(C)(CC(Cn6ncc(-c7ccc(N8CCc9cccc(C(=O)Nc%10nc%11ccccc%11s%10)c9C8)nc7C(=O)O)c6C)(C5)C3)C4)CCS(=O)(=O)O)cc2)C(C)C)N2C(=O)C=CC2=O)nn1] |
| 2.151 | YR | 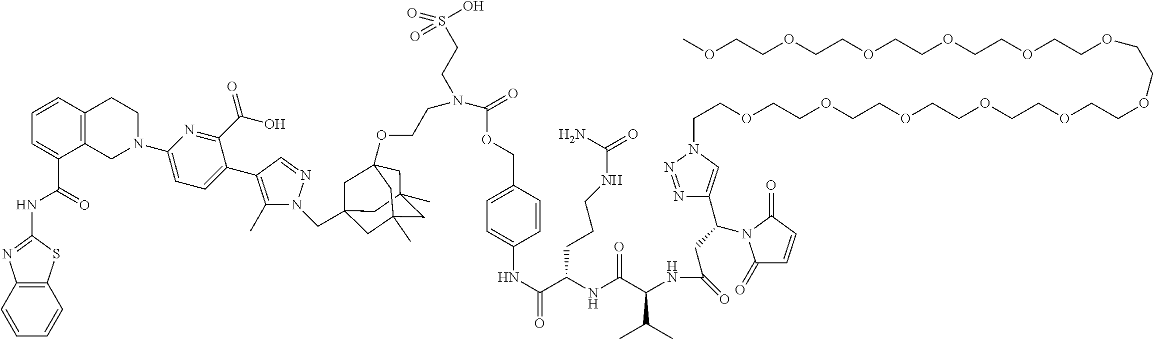 [SMILES: COCCOCCOCCOCCOCCOCCOCCOCCOCCOCCOCCOCCn1cc([C@@H](CC(=O)N[C@H](C(=O)N[C@@H](CCCNC(N)=O)C(=O)Nc2ccc(COC(=O)N(CCOC34CC5(C)CC(C)(CC(Cn6ncc(-c7ccc(N8CCc9cccc(C(=O)Nc%10nc%11ccccc%11s%10)c9C8)nc7C(=O)O)c6C)(C5)C3)C4)CCS(=O)(=O)O)cc2)C(C)C)N2C(=O)C=CC2=O)nn1] |
| 2.152 | YS | 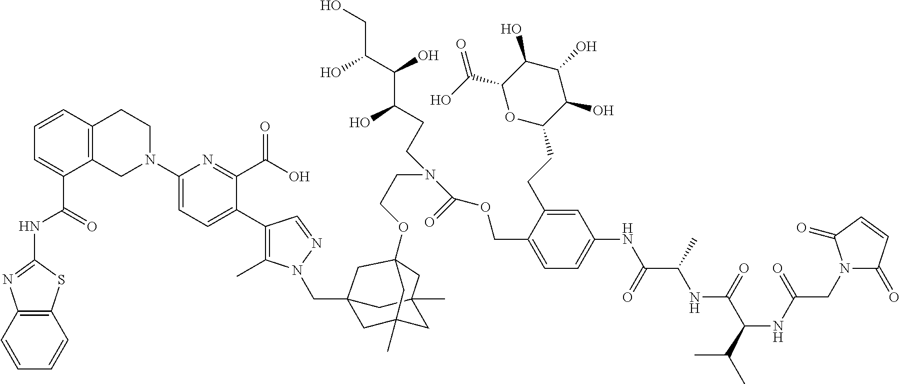 [SMILES: Cc1c(-c2ccc(N3CCc4cccc(C(=O)Nc5nc6ccccc6s5)c4C3)nc2C(=O)O)cnn1CC12CC3(C)CC(C)(C1)CC(OCCN(CC[C@@H](O)[C@H](O)[C@H](O)CO)C(=O)OCc1ccc(NC(=O)[C@H](C)NC(=O)[C@@H](NC(=O)CN4C(=O)C=CC4=O)C(C)C)cc1CC[C@@H]1O[C@H](C(=O)O)[C@@H](O)[C@H](O)[C@H]1O)(C3)C2] |
| 2.153 | YY | 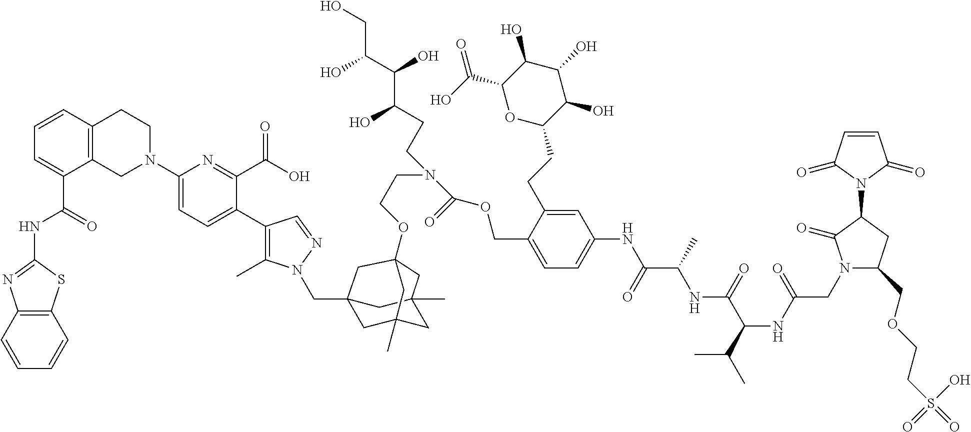 [SMILES: Cc1c(-c2ccc(N3CCc4cccc(C(=O)Nc5nc6ccccc6s5)c4C3)nc2C(=O)O)cnn1CC12CC3(C)CC(C)(C1)CC(OCCN(CC[C@@H](O)[C@H](O)[C@H](O)CO)C(=O)OCc1ccc(NC(=O)[C@H](C)NC(=O)[C@@H](NC(=O)CN4C(=O)[C@@H](N5C(=O)C=CC5=O)C[C@H]4COCCS(=O)(=O)O)C(C)C)cc1CC[C@@H]1O[C@H](C(=O)O)[C@@H](O)[C@H](O)[C@H]1O)(C3)C2] |
| 2.154 | YT | 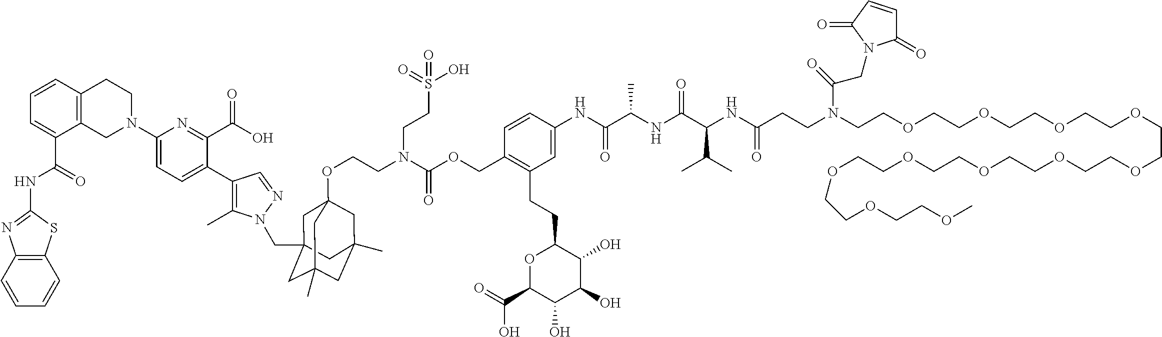 [SMILES: COCCOCCOCCOCCOCCOCCOCCOCCOCCOCCOCCN(CCC(=O)N[C@H](C(=O)N[C@@H](C)C(=O)Nc1ccc(COC(=O)N(CCOC23CC4(C)CC(C)(CC(Cn5ncc(-c6ccc(N7CCc8cccc(C(=O)Nc9nc%10ccccc%10s9)c8C7)nc6C(=O)O)c5C)(C4)C2)C3)CCS(=O)(=O)O)c(CC[C@@H]2O[C@H](C(=O)O)[C@@H](O)[C@H](O)[C@H]2O)c1)C(C)C)C(=O)CN1C(=O)C=CC1=O] |
| 2.155 | YU | 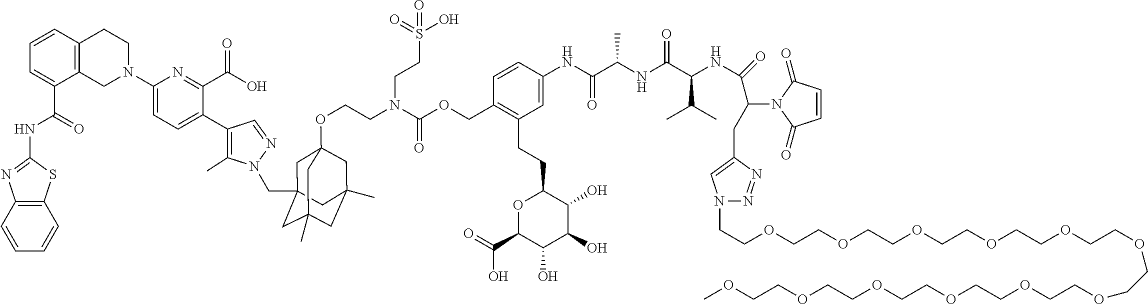 [SMILES: COCCOCCOCCOCCOCCOCCOCCOCCOCCOCCOCCOCCn1cc(CC(C(=O)N[C@H](C(=O)N[C@@H](C)C(=O)Nc2ccc(COC(=O)N(CCOC34CC5(C)CC(C)(CC(Cn6ncc(-c7ccc(N8CCc9cccc(C(=O)Nc%10nc%11ccccc%11s%10)c9C8)nc7C(=O)O)c6C)(C5)C3)C4)CCS(=O)(=O)O)c(CC[C@@H]3O[C@H](C(=O)O)[C@@H](O)[C@H](O)[C@H]3O)c2)C(C)C)N2C(=O)C=CC2=O)nn1] |
| 2.156 | YV | 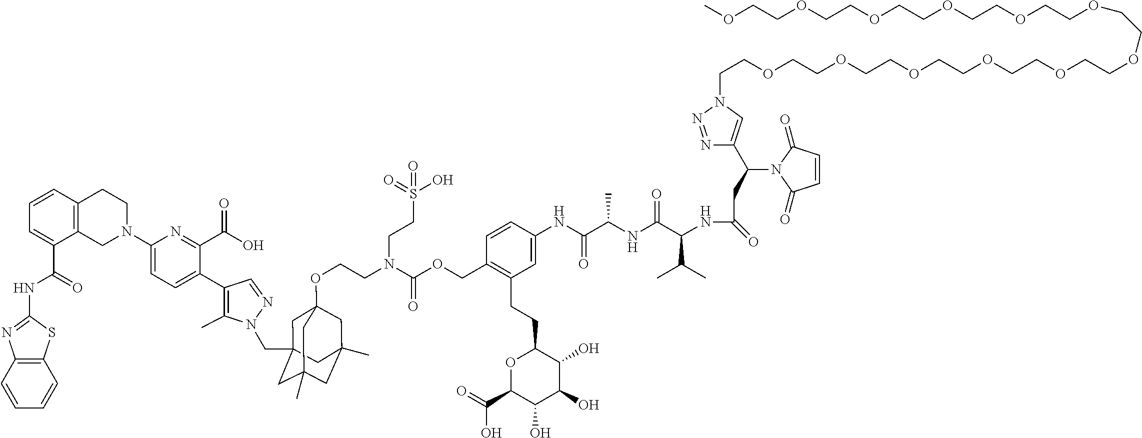 [SMILES: COCCOCCOCCOCCOCCOCCOCCOCCOCCOCCOCCOCCn1cc([C@H](CC(=O)N[C@H](C(=O)N[C@@H](C)C(=O)Nc2ccc(COC(=O)N(CCOC34CC5(C)CC(C)(CC(Cn6ncc(-c7ccc(N8CCc9cccc(C(=O)Nc%10nc%11ccccc%11s%10)c9C8)nc7C(=O)O)c6C)(C5)C3)C4)CCS(=O)(=O)O)c(CC[C@@H]3O[C@H](C(=O)O)[C@@H](O)[C@H](O)[C@H]3O)c2)C(C)C)N2C(=O)C=CC2=O)nn1] |
| 2.157 | YW | 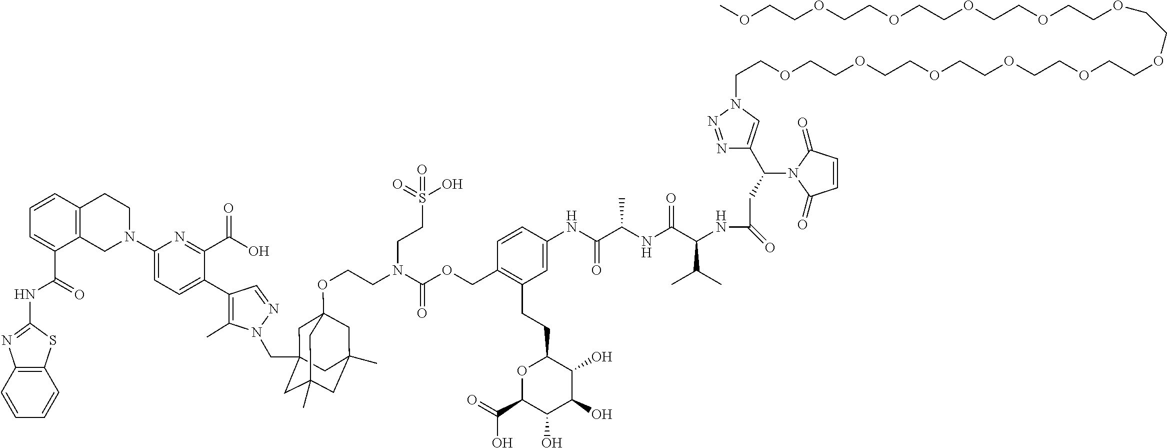 [SMILES: COCCOCCOCCOCCOCCOCCOCCOCCOCCOCCOCCOCCn1cc([C@@H](CC(=O)N[C@H](C(=O)N[C@@H](C)C(=O)Nc2ccc(COC(=O)N(CCOC34CC5(C)CC(C)(CC(Cn6ncc(-c7ccc(N8CCc9cccc(C(=O)Nc%10nc%11ccccc%11s%10)c9C8)nc7C(=O)O)c6C)(C5)C3)C4)CCS(=O)(=O)O)c(CC[C@@H]3O[C@H](C(=O)O)[C@@H](O)[C@H](O)[C@H]3O)c2)C(C)C)N2C(=O)C=CC2=O)nn1] |
| 2.158 | ZB | 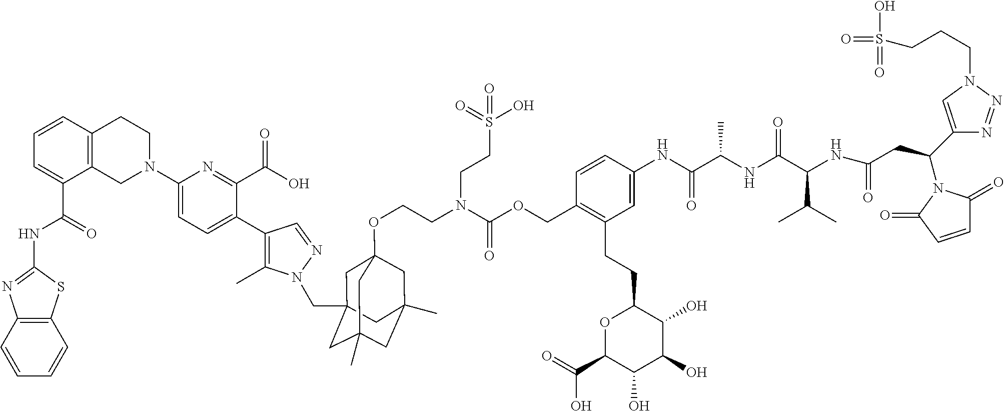 [SMILES: Cc1c(-c2ccc(N3CCc4cccc(C(=O)Nc5nc6ccccc6s5)c4C3)nc2C(=O)O)cnn1CC12CC3(C)CC(C)(C1)CC(OCCN(CCS(=O)(=O)O)C(=O)OCc1ccc(NC(=O)[C@H](C)NC(=O)[C@@H](NC(=O)C[C@@H](c4cn(CCCS(=O)(=O)O)nn4)N4C(=O)C=CC4=O)C(C)C)cc1CC[C@@H]1O[C@H](C(=O)O)[C@@H](O)[C@H](O)[C@H]1O)(C3)C2] |
| 2.159 | ZC | 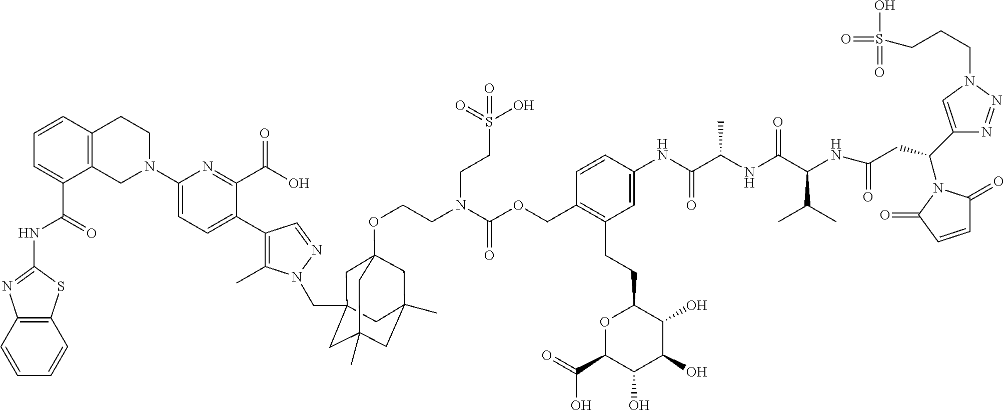 [SMILES: Cc1c(-c2ccc(N3CCc4cccc(C(=O)Nc5nc6ccccc6s5)c4C3)nc2C(=O)O)cnn1CC12CC3(C)CC(C)(C1)CC(OCCN(CCS(=O)(=O)O)C(=O)OCc1ccc(NC(=O)[C@H](C)NC(=O)[C@@H](NC(=O)C[C@H](c4cn(CCCS(=O)(=O)O)nn4)N4C(=O)C=CC4=O)C(C)C)cc1CC[C@@H]1O[C@H](C(=O)O)[C@@H](O)[C@H](O)[C@H]1O)(C3)C2] |
| 2.160 | ZJ | 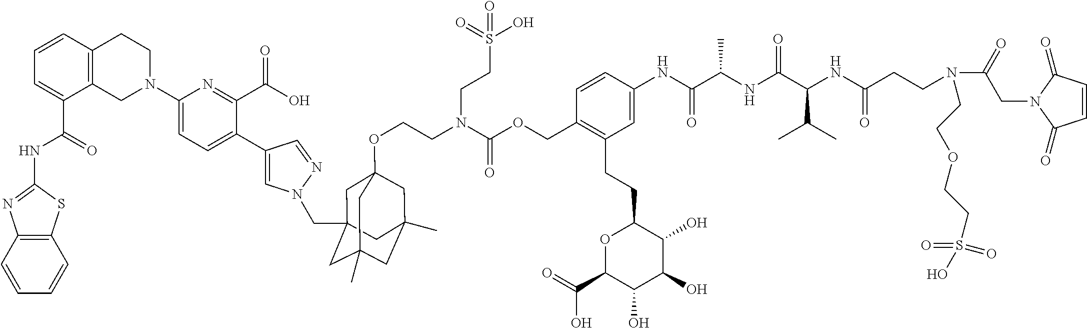 [SMILES: CC(C)[C@H](NC(=O)CCN(CCOCCS(=O)(=O)O)C(=O)CN1C(=O)C=CC1=O)C(=O)N[C@@H](C)C(=O)Nc1ccc(COC(=O)N(CCOC23CC4(C)CC(C)(CC(Cn5cc(-c6ccc(N7CCc8cccc(C(=O)Nc9nc%10ccccc%10s9)c8C7)nc6C(=O)O)cn5)(C4)C2)C3)CCS(=O)(=O)O)c(CC[C@@H]2O[C@H](C(=O)O)[C@@H](O)[C@H](O)[C@H]2O)c1] |
| 2.161 | ZE | 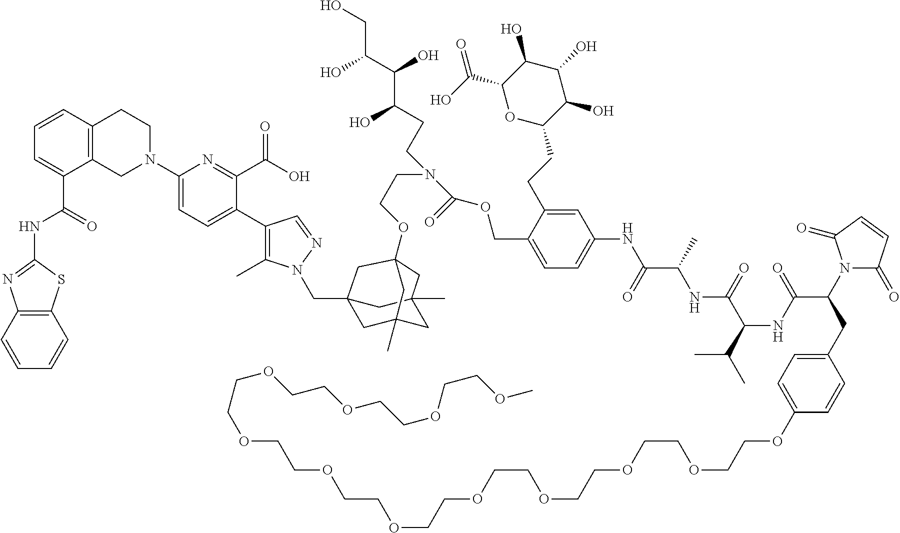 [SMILES: COCCOCCOCCOCCOCCOCCOCCOCCOCCOCCOCCOc1ccc(C[C@@H](C(=O)N[C@H](C(=O)N[C@@H](C)C(=O)Nc2ccc(COC(=O)N(CCOC34CC5(C)CC(C)(CC(Cn6ncc(-c7ccc(N8CCc9cccc(C(=O)Nc%10nc%11ccccc%11s%10)c9C8)nc7C(=O)O)c6C)(C5)C3)C4)CC[C@@H](O)[C@H](O)[C@H](O)CO)c(CC[C@@H]3O[C@H](C(=O)O)[C@@H](O)[C@H](O)[C@H]3O)c2)C(C)C)N2C(=O)C=CC2=O)cc1] |
| 2.162 | ZS | 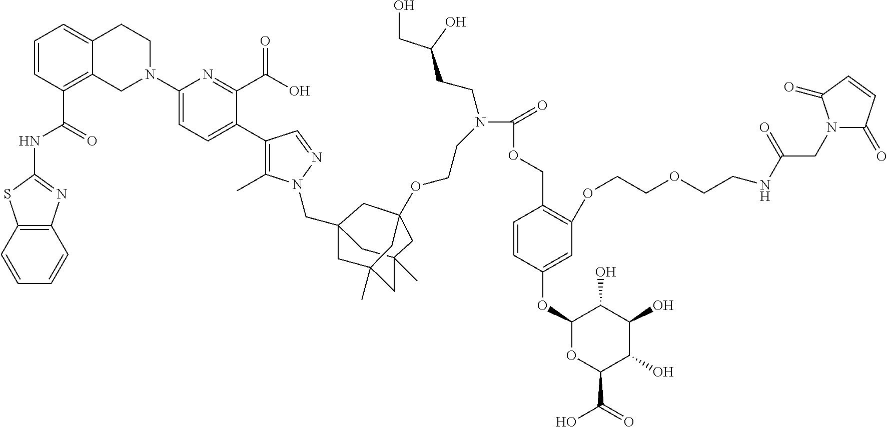 [SMILES: Cc1c(-c2ccc(N3CCc4cccc(C(=O)Nc5nc6ccccc6s5)c4C3)nc2C(=O)O)cnn1CC12CC3(C)CC(C)(C1)CC(OCCN(CC[C@H](O)CO)C(=O)OCc1ccc(O[C@@H]4O[C@H](C(=O)O)[C@@H](O)[C@H](O)[C@H]4O)cc1OCCOCCNC(=O)CN1C(=O)C=CC1=O)(C3)C2] |
| 2.163 | ZW | 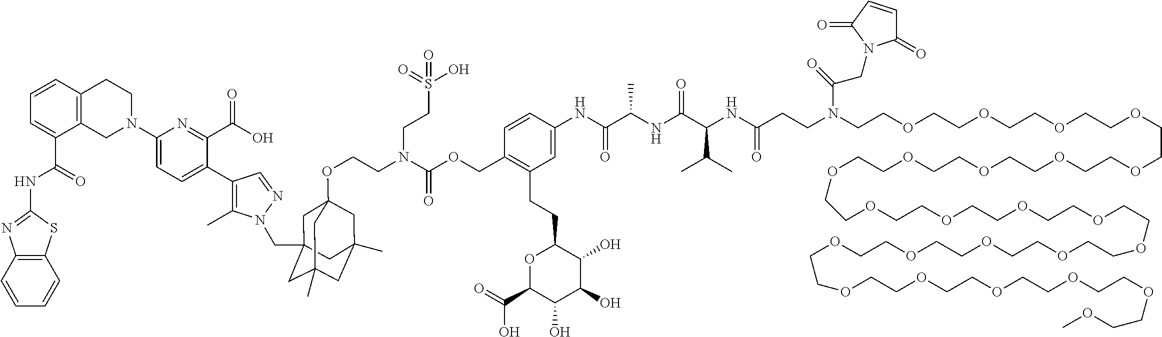 [SMILES: COCCOCCOCCOCCOCCOCCOCCOCCOCCOCCOCCOCCOCCOCCOCCOCCOCCOCCOCCOCCOCCOCCOCCOCCN(CCC(=O)N[C@H](C(=O)N[C@@H](C)C(=O)Nc1ccc(COC(=O)N(CCOC23CC4(C)CC(C)(CC(Cn5ncc(-c6ccc(N7CCc8cccc(C(=O)Nc9nc%10ccccc%10s9)c8C7)nc6C(=O)O)c5C)(C4)C2)C3)CCS(=O)(=O)O)c(CC[C@@H]2O[C@H](C(=O)O)[C@@H](O)[C@H](O)[C@H]2O)c1)C(C)C)C(=O)CN1C(=O)C=CC1=O] |
| 2.164 | ZX | 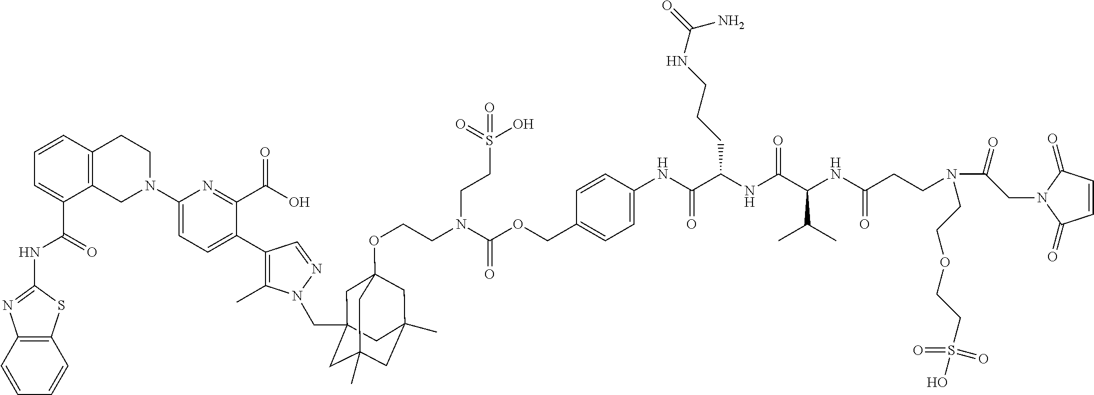 [SMILES: Cc1c(-c2ccc(N3CCc4cccc(C(=O)Nc5nc6ccccc6s5)c4C3)nc2C(=O)O)cnn1CC12CC3(C)CC(C)(C1)CC(OCCN(CCS(=O)(=O)O)C(=O)OCc1ccc(NC(=O)[C@H](CCCNC(N)=O)NC(=O)[C@@H](NC(=O)CCN(CCOCCS(=O)(=O)O)C(=O)CN4C(=O)C=CC4=O)C(C)C)cc1)(C3)C2] |
| 2.166 | AAA | 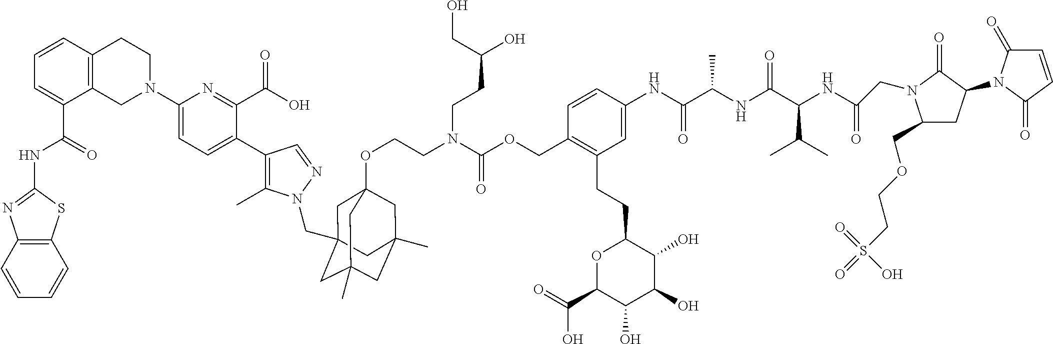 [SMILES: Cc1c(-c2ccc(N3CCc4cccc(C(=O)Nc5nc6ccccc6s5)c4C3)nc2C(=O)O)cnn1CC12CC3(C)CC(C)(C1)CC(OCCN(CC[C@H](O)CO)C(=O)OCc1ccc(NC(=O)[C@H](C)NC(=O)[C@@H](NC(=O)CN4C(=O)[C@@H](N5C(=O)C=CC5=O)C[C@H]4COCCS(=O)(=O)O)C(C)C)cc1CC[C@@H]1O[C@H](C(=O)O)[C@@H](O)[C@H](O)[C@H]1O)(C3)C2] |
| 2.167 | AAD | 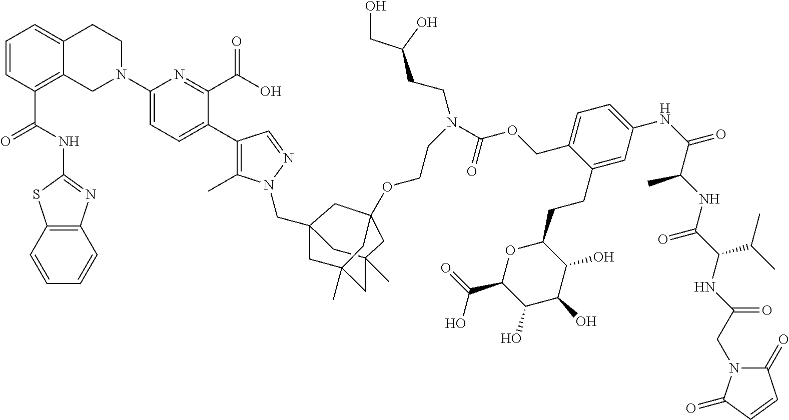 [SMILES: Cc1c(-c2ccc(N3CCc4cccc(C(=O)Nc5nc6ccccc6s5)c4C3)nc2C(=O)O)cnn1CC12CC3(C)CC(C)(C1)CC(OCCN(CC[C@H](O)CO)C(=O)OCc1ccc(NC(=O)[C@H](C)NC(=O)[C@@H](NC(=O)CN4C(=O)C=CC4=O)C(C)C)cc1CC[C@@H]1O[C@H](C(=O)O)[C@@H](O)[C@H](O)[C@H]1O)(C3)C2] |
| 2.168 | AAE | 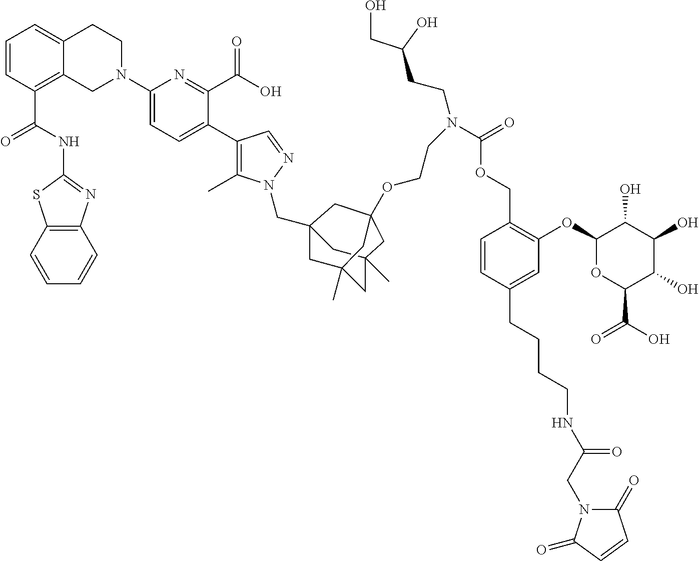 [SMILES: Cc1c(-c2ccc(N3CCc4cccc(C(=O)Nc5nc6ccccc6s5)c4C3)nc2C(=O)O)cnn1CC12CC3(C)CC(C)(C1)CC(OCCN(CC[C@H](O)CO)C(=O)OCc1ccc(CCCCNC(=O)CN4C(=O)C=CC4=O)cc1O[C@@H]1O[C@H](C(=O)O)[C@@H](O)[C@H](O)[C@H]1O)(C3)C2] |
| 2.169 | ABG | 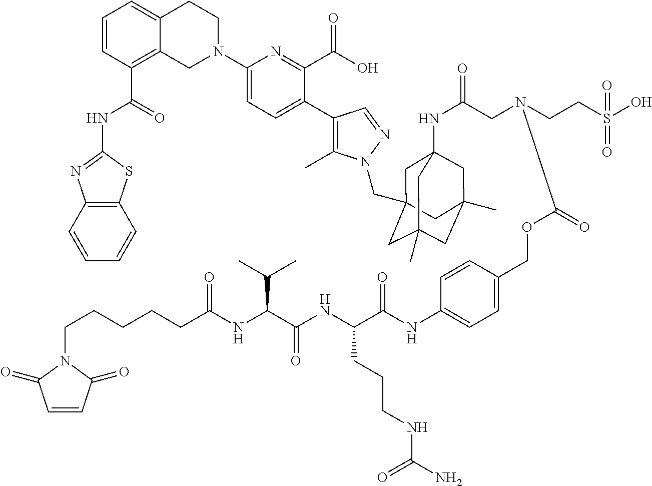 [SMILES: Cc1c(-c2ccc(N3CCc4cccc(C(=O)Nc5nc6ccccc6s5)c4C3)nc2C(=O)O)cnn1CC12CC3(C)CC(C)(C1)CC(NC(=O)CN(CCS(=O)(=O)O)C(=O)OCc1ccc(NC(=O)[C@H](CCCNC(N)=O)NC(=O)[C@@H](NC(=O)CCCCCN4C(=O)C=CC4=O)C(C)C)cc1)(C3)C2] |
| 2.170 | ABL | 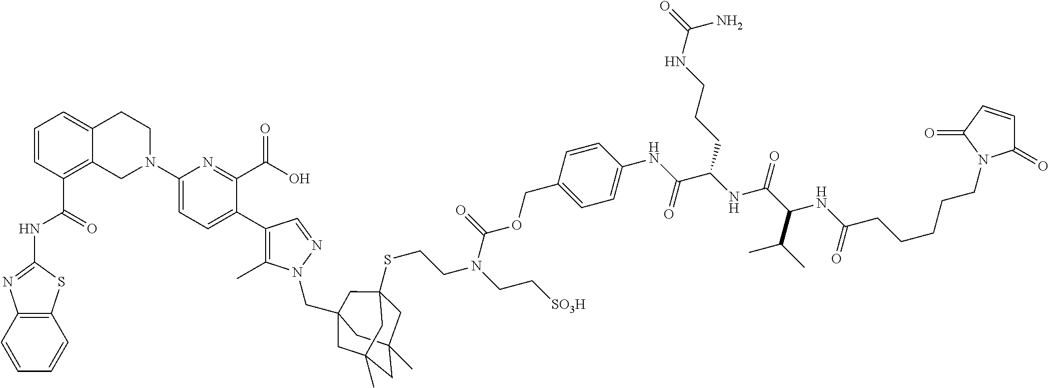 [SMILES: Cc1c(-c2ccc(N3CCc4cccc(C(=O)Nc5nc6ccccc6s5)c4C3)nc2C(=O)O)cnn1CC12CC3(C)CC(C)(C1)CC(SCCN(CCS(=O)(=O)O)C(=O)OCc1ccc(NC(=O)[C@H](CCCNC(N)=O)NC(=O)[C@@H](NC(=O)CCCCCN4C(=O)C=CC4=O)C(C)C)cc1)(C3)C2] |
| 2.171 | ABN | 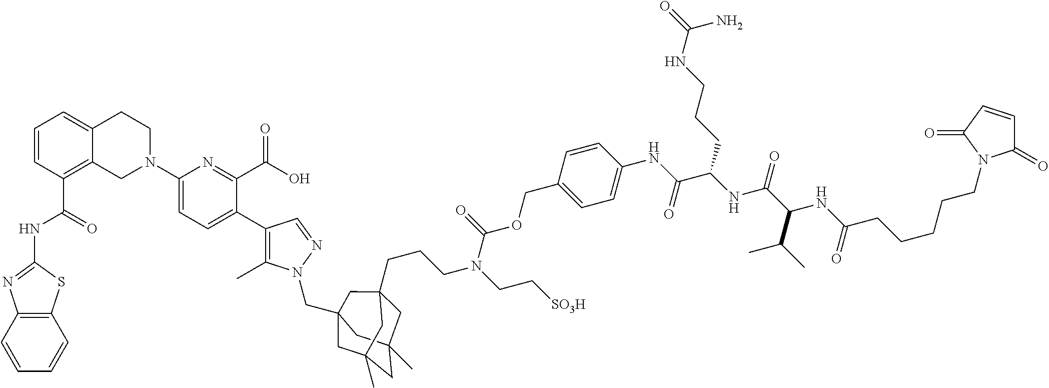 [SMILES: Cc1c(-c2ccc(N3CCc4cccc(C(=O)Nc5nc6ccccc6s5)c4C3)nc2C(=O)O)cnn1CC12CC3(C)CC(C)(CC(CCCN(CCS(=O)(=O)O)C(=O)OCc4ccc(NC(=O)[C@H](CCCNC(N)=O)NC(=O)[C@@H](NC(=O)CCCCCN5C(=O)C=CC5=O)C(C)C)cc4)(C3)C1)C2] |
| 2.172 | AAF | 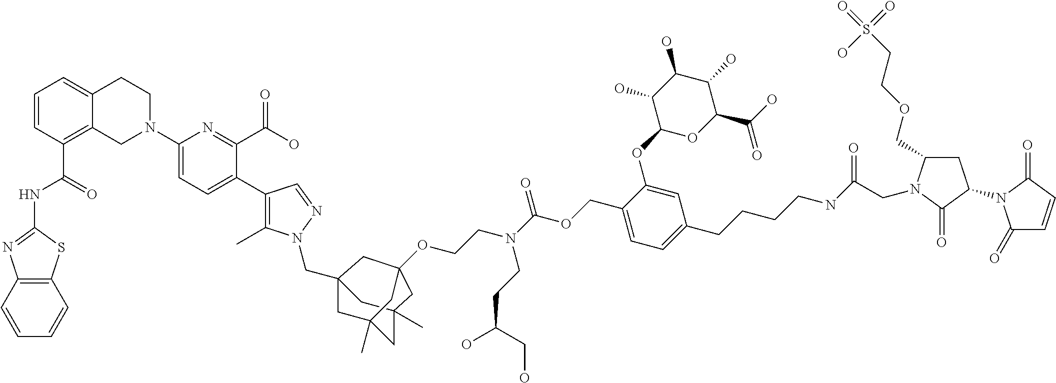 [SMILES: Cc1c(-c2ccc(N3CCc4cccc(C(=O)Nc5nc6ccccc6s5)c4C3)nc2C(=O)O)cnn1CC12CC3(C)CC(C)(C1)CC(OCCN(CC[C@H](O)CO)C(=O)OCc1ccc(CCCCNC(=O)CN4C(=O)[C@@H](N5C(=O)C=CC5=O)C[C@H]4COCCS(=O)(=O)O)cc1O[C@@H]1O[C@H](C(=O)O)[C@@H](O)[C@H](O)[C@H]1O)(C3)C2] |
| 2.173 | ABO | 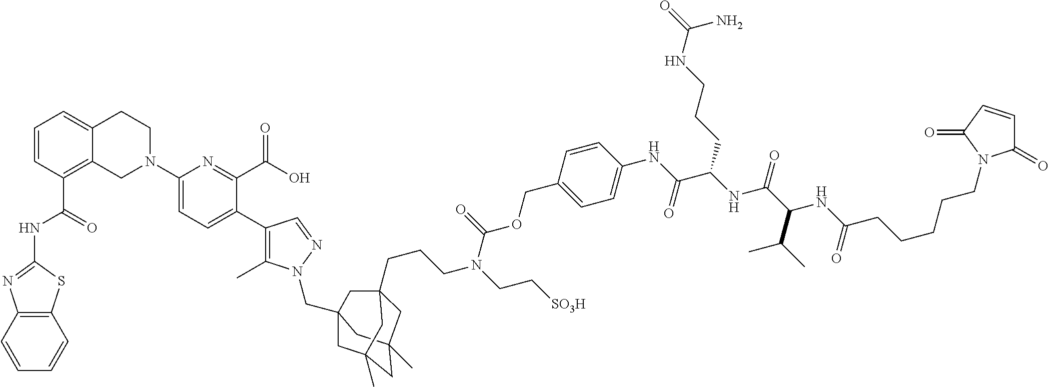 [SMILES: Cc1c(-c2ccc(N3CCc4cccc(C(=O)Nc5nc6ccccc6s5)c4C3)nc2C(=O)O)cnn1CC12CC3(C)CC(C)(CC(CCCN(CCS(=O)(=O)O)C(=O)OCc4ccc(NC(=O)[C@H](CCCNC(N)=O)NC(=O)[C@@H](NC(=O)CCCCCN5C(=O)C=CC5=O)C(C)C)cc4)(C3)C1)C2] |
| 2.174 | ABM | 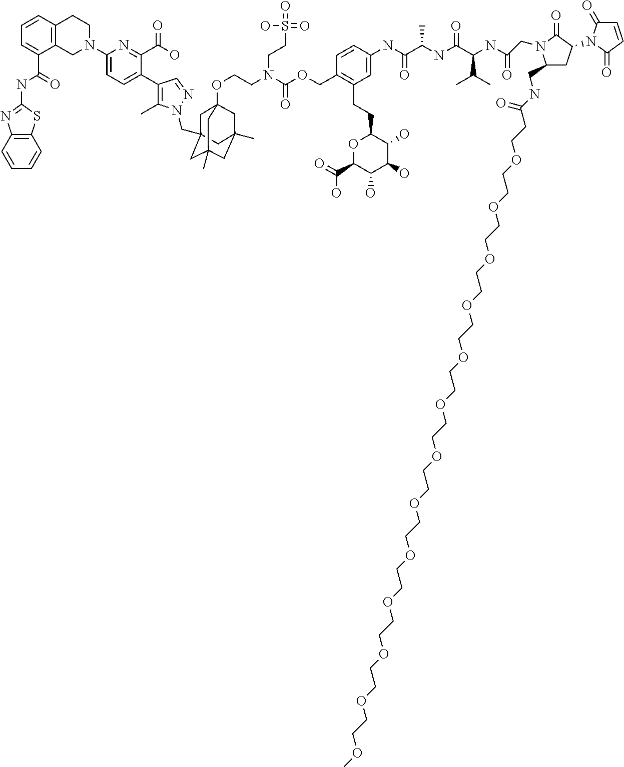 [SMILES: COCCOCCOCCOCCOCCOCCOCCOCCOCCOCCOCCOCCOCCC(=O)NC[C@@H]1C[C@@H](N2C(=O)C=CC2=O)C(=O)N1CC(=O)N[C@H](C(=O)N[C@@H](C)C(=O)Nc1ccc(COC(=O)N(CCOC23CC4(C)CC(C)(CC(Cn5ncc(-c6ccc(N7CCc8cccc(C(=O)Nc9nc%10ccccc%10s9)c8C7)nc6C(=O)O)c5C)(C4)C2)C3)CCS(=O)(=O)O)c(CC[C@@H]2O[C@H](C(=O)O)[C@@H](O)[C@H](O)[C@H]2O)c1)C(C)C] |
| 2.175 | ABU | 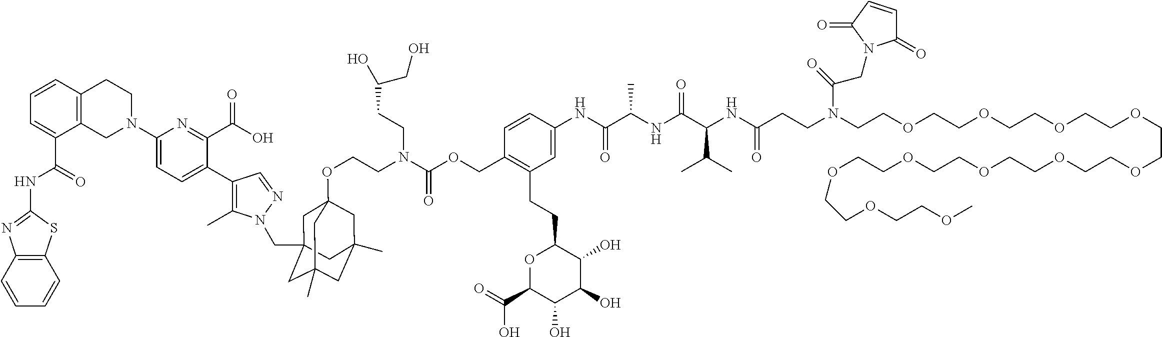 |
| 2.176 | ABV | 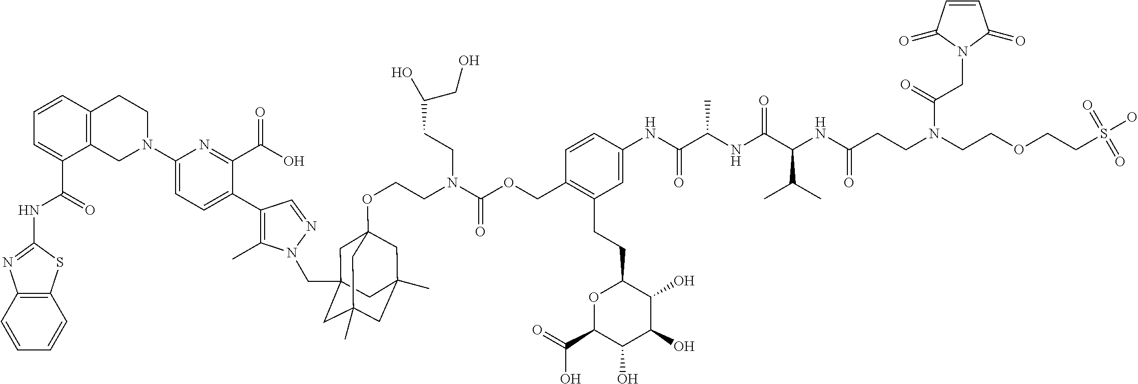 |

In certain embodiments, an ADC, or a pharmaceutically acceptable salt thereof, is formed by contacting an antibody that binds a cell surface receptor or tumor associated antigen expressed on a tumor cell with a synthon, under conditions in which the synthon covalently links to the antibody, wherein the synthon is selected from the group consisting of synthon examples 2.1, 2.2, 2.4, 2.5, 2.6, 2.7, 2.8, 2.9, 2.10, 2.11, 2.12, 2.13, 2.14, 2.15, 2.16, 2.17, 2.18, 2.19, 2.20, 2.21, 2.22, 2.23, 2.24, 2.25, 2.26, 2.27, 2.28, 2.29, 2.30, 2.31, 2.32, 2.33, 2.34, 2.35, 2.36, 2.37, 2.38, 2.39, 2.40, 2.41, 2.42, 2.43, 2.44, 2.45, 2.46, 2.47, 2.48, 2.49, 2.50, 2.51, 2.52, 2.53, 2.54, 2.55, 2.56, 2.57, 2.58, 2.59, 2.60, 2.61, 2.62, 2.63, 2.64, 2.65, 2.66, 2.67, 2.68, 2.69, 2.77, 2.78, 2.79, 2.80, 2.81, 2.82, 2.83, 2.84, 2.85, 2.86, 2.87, 2.88, 2.89, 2.90, 2.91, 2.92, 2.93, 2.94, 2.95, 2.96, 2.97, 2.98, 2.101, 2.102, 2.103, 2.104, 2.105, 2.106, 2.107, 2.108, 2.109, 2.110, 2.111, 2.112, 2.113, 2.114, 2.115, 2.116, 2.117, 2.118, 2.119, 2.120, 2.121, 2.122, 2.123, 2.124, 2.125, 2.126, 2.127, 2.128, 2.129, 2.130, 2.131, 2.132, 2.133, 2.134, 2.135, 2.136, 2.137, 2.138, 2.139, 2.140, 2.141, 2.142, 2.143, 2.144, 2.145, 2.146, 2.147, 2.148, 2.149, 2.150, 2.151, 2.152, 2.153, 2.154, 2.155, 2.156, 2.157, 2.158, 2.159, 2.160, 2.161, 2.162, 2.163, 2.164, 2.166, 2.167, 2.168, 2.169, 2.170, 2.171, 2.172, 2.173, 2.174, 2.175, and 2.176, or a pharmaceutically acceptable salt thereof.

### 4.8. Antibody Drug Conjugates

Bcl-xL inhibitory activity of ADCs described herein may be confirmed in cellular assays with appropriate target cells and/or in vivo assays. Specific assays that may be used to confirm activity of ADCs that target EGFR, EpCAM or NCAM1 are provided in Examples 8 and 9, respectively. Generally, ADCs will exhibit an EC₅₀ of less than about 5000 nM in such a cellular assay, although the ADCs may exhibit significantly lower EC₅₀s, for example, less than about 500, 300, or even 100 nM. Similar cellular assays with cells expressing specific target antigens may be used to confirm the Bcl-xL inhibitory activity of ADCs targeting other antigens.

### 4.9. Methods of Synthesis

The Bcl-xL inhibitors and synthons described herein may be synthesized using standard, known techniques of organic chemistry. General schemes for synthesizing Bcl-xL inhibitors and synthons that may be used as-is or modified to synthesize the full scope of Bcl-xL inhibitors and synthons described herein are provided below. Specific methods for synthesizing exemplary Bcl-xL inhibitors and synthons that may be useful for guidance are provided in the Examples section. ADCs may likewise be prepared by standard methods, such as methods analogous to those described in Hamblett et al., 2004, “Effects of Drug Loading on the Antitumor Activity of a Monoclonal Antibody Drug Conjugate”, Clin. Cancer Res. 10:7063-7070; Doronina et al., 2003, “Development of potent and highly efficacious monoclonal antibody auristatin conjugates for cancer therapy,” Nat. Biotechnol. 21(7):778-784; and Francisco et al., 2003, Blood 102:1458-1465. For example, ADCs with four drugs per antibody may be prepared by partial reduction of the antibody with an excess of a reducing reagent such as DTT or TCEP at 37° C. for 30 min, then the buffer exchanged by elution through SEPHADEX® G-25 resin with 1 mM DTPA in DPBS. The eluent is diluted with further DPBS, and the thiol concentration of the antibody may be measured using 5,5′-dithiobis(2-nitrobenzoic acid) [Ellman's reagent]. An excess, for example 5-fold, of a linker-drug synthon is added at 4° C. for 1 hr, and the conjugation reaction may be quenched by addition of a substantial excess, for example 20-fold, of cysteine. The resulting ADC mixture may be purified on SEPHADEX G-25 equilibrated in PBS to remove unreacted synthons, desalted if desired, and purified by size-exclusion chromatography. The resulting ADC may then be then sterile filtered, for example, through a 0.2 μm filter, and lyophilized if desired for storage. In certain embodiments, all of the interchain cysteine disulfide bonds are replaced by linker-drug conjugates. One embodiment pertains to a method of making an ADC, comprising contacting a synthon described herein with an antibody under conditions in which the synthon covalently links to the antibody.

Specific methods for synthesizing exemplary ADCs that may be used to synthesize the full range of ADCs described herein are provided in the Examples section.

### 4.9.1. General Methods for Synthesizing Bcl-xL Inhibitors

In the schemes below, the various substituents Ar¹, Ar², Z¹, R⁴, R^{11a} and R^{11b} are as defined in the Detailed Description section.

### 4.9.1.1. Synthesis of Compound (6)

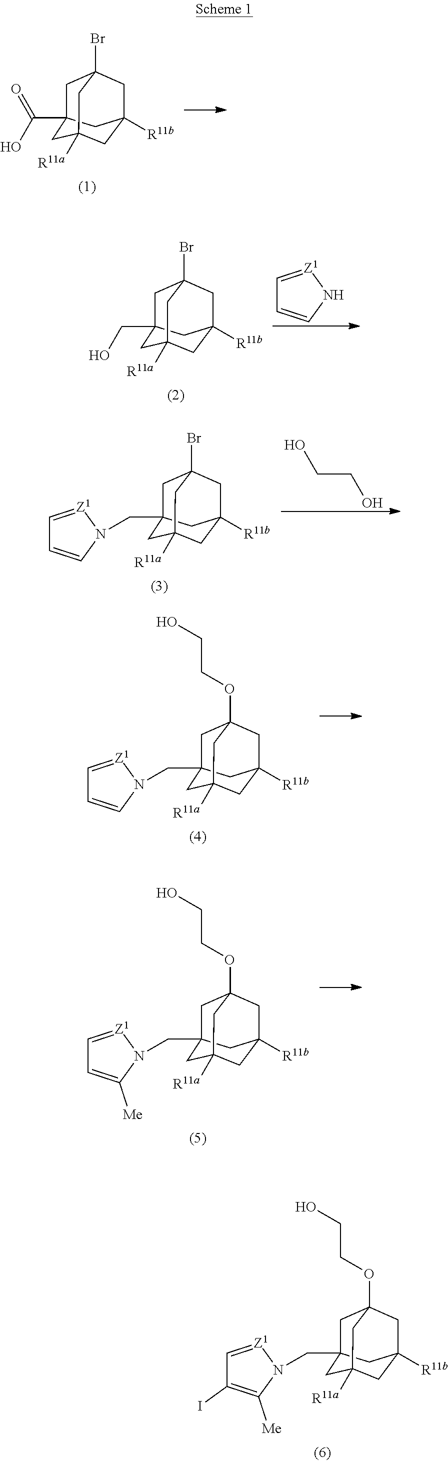

The synthesis of an intermediate (6) is described in Scheme 1. Compound (1) can be treated with BH₃·THF to provide compound (2). The reaction is typically performed at ambient temperature in a solvent, such as, but not limited to, tetrahydrofuran. Compound (3) can be prepared by treating compound (2) with

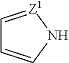

in the presence of cyanomethylenetributylphosphorane. The reaction is typically performed at an elevated temperature in a solvent such as, but not limited to, toluene. Compound (3) can be treated with ethane-1,2-diol in the presence of a base such as, but not limited to, triethylamine, to provide compound (4). The reaction is typically performed at an elevated temperature, and the reaction may be performed under microwave conditions. Compound (4) can be treated with a strong base, such as, but not limited to, n-butyllithium, followed by the addition of iodomethane, to provide compound (5). The addition and reaction is typically performed in a solvent such as, but not limited to, tetrahydrofuran, at a reduced temperature before warming up to ambient temperature for work up. Compound (5) can be treated with N-iodosuccinimide to provide compound (6). The reaction is typically performed at ambient temperature is a solvent such as, but not limited to, N,N-dimethylformamide.

### 4.9.1.2. Synthesis of Compound (12)

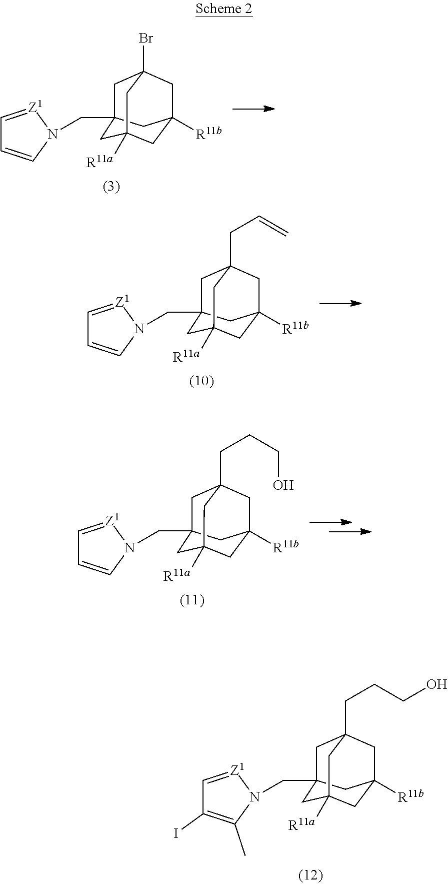

The synthesis of intermediate (12) is described in Scheme 2. Compound (3) can be treated with tri-n-butyl-allylstannane in the presence of ZnCl₂·Et₂O or N, N′-azoisobutyronitrile (AIBN) to provide compound (10) (Yamamoto et al., 1998, Heterocycles 47:765-780). The reaction is typically performed at −78° C. in a solvent, such as, but not limited to dichloromethane. Compound (10) can be treated under standard conditions known in the art for hydroboration/oxidation to provide compound (11). For example, treatment of compound (10) with a reagent such as BH₃·THF in a solvent such as, but not limited to, tetrahydrofuran followed by treatment of the intermediate alkylborane adduct with an oxidant such as, but not limited to, hydrogen peroxide in the presence of a base such as, but not limited to, sodium hydroxide would provide compound (11) (Brown et al., 1968, J. Am. Chem. Soc. 86:397). Typically the addition of BH₃·THF is performed at low temperature before warming to ambient temperature, which is followed by the addition of hydrogen peroxide and sodium hydroxide to generate the alcohol product. Compound (12) can be generated according to Scheme 1, as previously described for compound (6).

### 4.9.1.3. Synthesis of Compound (15)

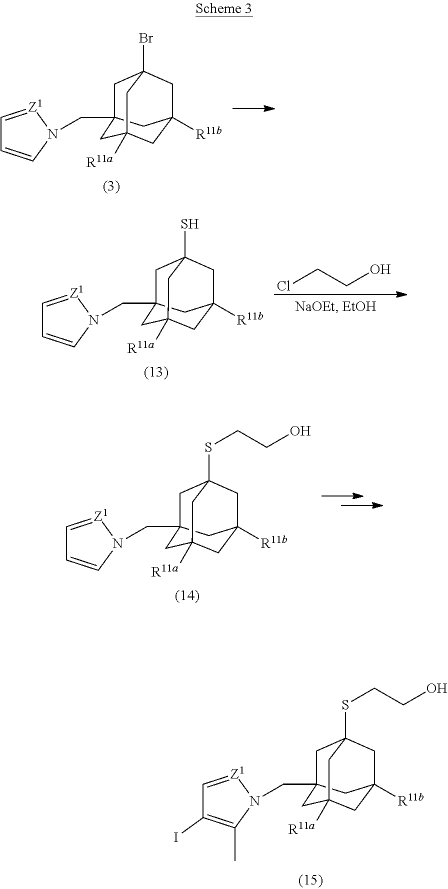

The synthesis of intermediate (15) is described in Scheme 3. Compound (3) can be reacted with thiourea in a solvent mixture of acetic acid and 48% aqueous HBr solution at 100° C. to yield an intermediate that can be subsequently treated with sodium hydroxide in a solvent mixture such as, but not limited to, 20% v/v ethanol in water to provide compound (13). Compound (13) can be reacted with 2-chloroethanol in the presence of a base such as, but not limited to, sodium ethoxide to provide compound (14). The reaction is typically performed at ambient or elevated temperatures in a solvent such as, but not limited to, ethanol. Compound (15) can be generated according to Scheme 1, as previously described for compound (6).

### 4.9.1.4. Synthesis of Compound (22)

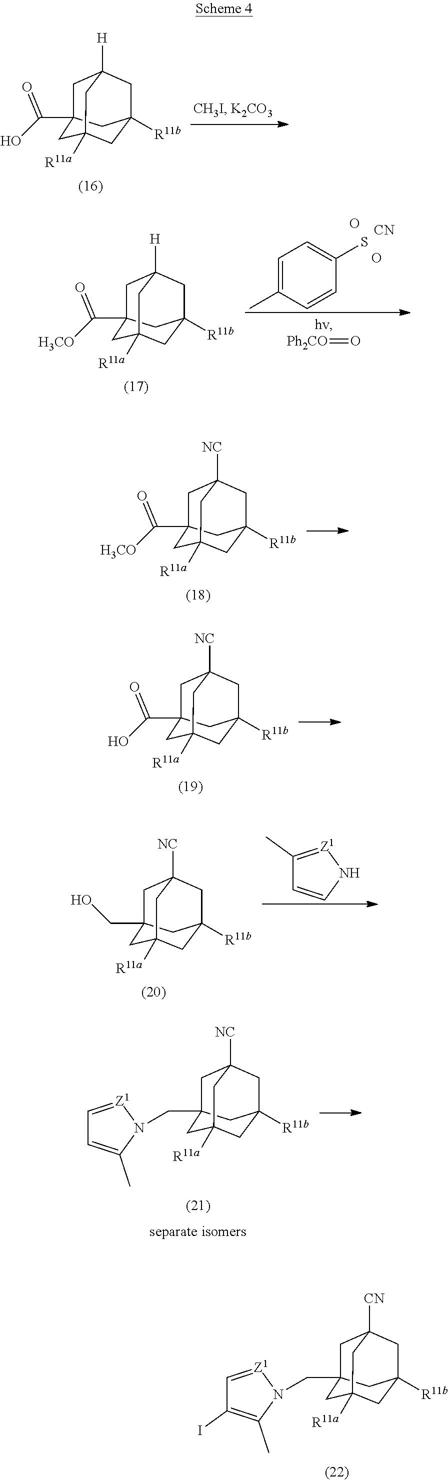

The synthesis of compound (22) is described in Scheme 4. Compound (16) can be reacted with iodomethane in the presence of a base such as, but not limited to, potassium carbonate to provide compound (17). The reaction is typically conducted at ambient or elevated temperature in a solvent such as, but not limited to, acetone or N,N-dimethylformamide. Compound (17) can be reacted under photochemical conditions with tosyl cyanide in the presence of benzophenone to provide compound (18) (see Kamijo et al., 2011, Org. Lett., 13:5928-5931). The reaction is typically run at ambient temperature in a solvent such as, but not limited to, acetonitrile or benzene using a Riko 100W medium pressure mercury lamp as the light source. Compound (18) can be reacted with lithium hydroxide in a solvent system such as, but not limited to, mixtures of water and tetrahydrofuran or water and methanol to provide compound (19). Compound (19) can be treated with BH₃·THF to provide compound (20). The reaction is typically performed at ambient temperature in a solvent, such as, but not limited to, tetrahydrofuran. Compound (21) can be prepared by treating compound (20) with

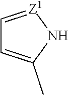

in the presence of cyanomethylenetributylphosphorane. The reaction is typically performed at an elevated temperature in a solvent such as, but not limited to, toluene. Compound (21) can be treated with N-iodosuccinimide to provide compound (22). The reaction is typically performed at ambient temperature is a solvent such as, but not limited to, N,N-dimethylformamide.

### 4.9.1.5. Synthesis of Compound (24)

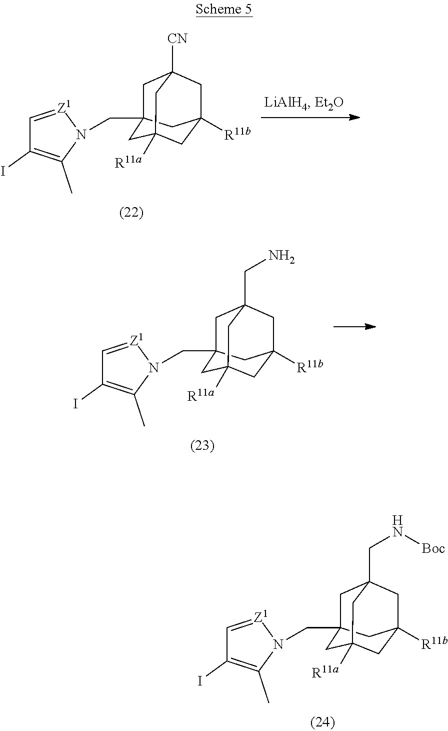

The synthesis of pyrazole compound (24), is described in Scheme 5. Compound (22) can be treated with a reducing agent such as, but not limited to, lithium aluminum hydride in a solvent such as, but not limited to, diethyl ether or tetrahydrofuran to provide compound (23). Typically the reaction is performed at 0° C. before warming to ambient or elevated temperature. Compound (23) can be reacted with di-tert-butyl dicarbonate under standard conditions described herein or in the literature to provide compound (24).

### 4.9.1.6. Synthesis of Compound (24a)

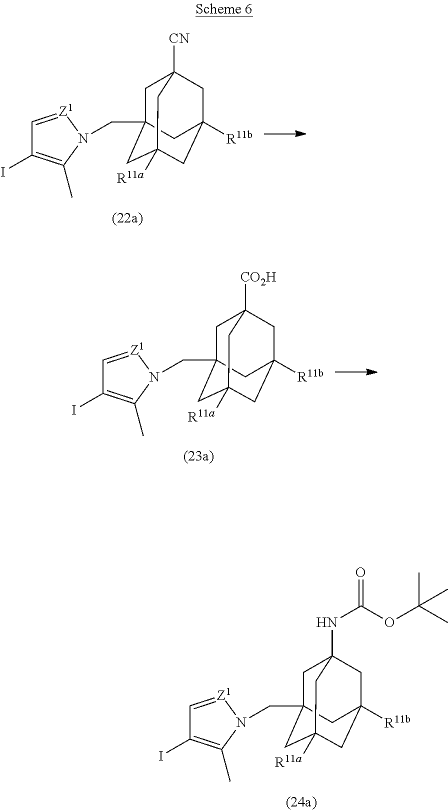

The synthesis of intermediate (24a) is described in Scheme 6. Compound (22a) can be hydrolyzed using conditions described in the literature to provide compound (23a). Typically the reaction is run in the presence of potassium hydroxide in a solvent such as, but not limited to, ethylene glycol at elevated temperatures (see Roberts et al., 1994, J. Org. Chem. 59:6464-6469; Yang et al, 2013, Org. Lett., 15:690-693). Compound (24a) can be made from compound (23a) by Curtius rearrangement using conditions described in the literature. For example, compound (23a) can be reacted with sodium azide in the presence of tetrabutylammonium bromide, zinc(II) triflate and di-tert-butyl dicarbonate to provide compound (24a) (see Lebel et al., Org. Lett., 2005, 7:4107-4110). Typically the reaction is run at elevated temperatures, preferably from 40-50° C., in a solvent such as, but not limited to, tetrahydrofuran.

### 4.9.1.7. Synthesis of Compound (29)

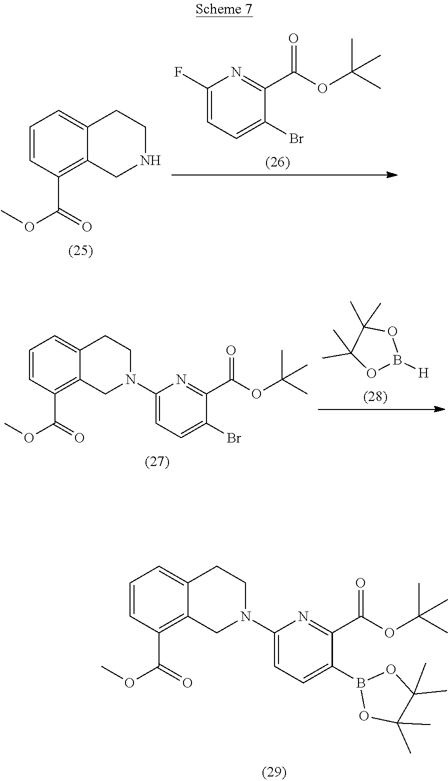

As shown in Scheme 7, compounds of formula (27) can be prepared by reacting compounds of formula (25) with tert-butyl 3-bromo-6-fluoropicolinate (26) in the presence of a base, such as, but not limited to, N,N-diisopropylethylamine, or triethylamine. The reaction is typically performed under an inert atmosphere at an elevated temperature in a solvent, such as, but not limited to, dimethyl sulfoxide. Compounds of formula (27) can be reacted with 4,4,5,5-tetramethyl-1,3,2-dioxaborolane (28), under borylation conditions described herein or in the literature to provide compounds of formula (29).

### 4.9.1.8. Synthesis of Compound (38)

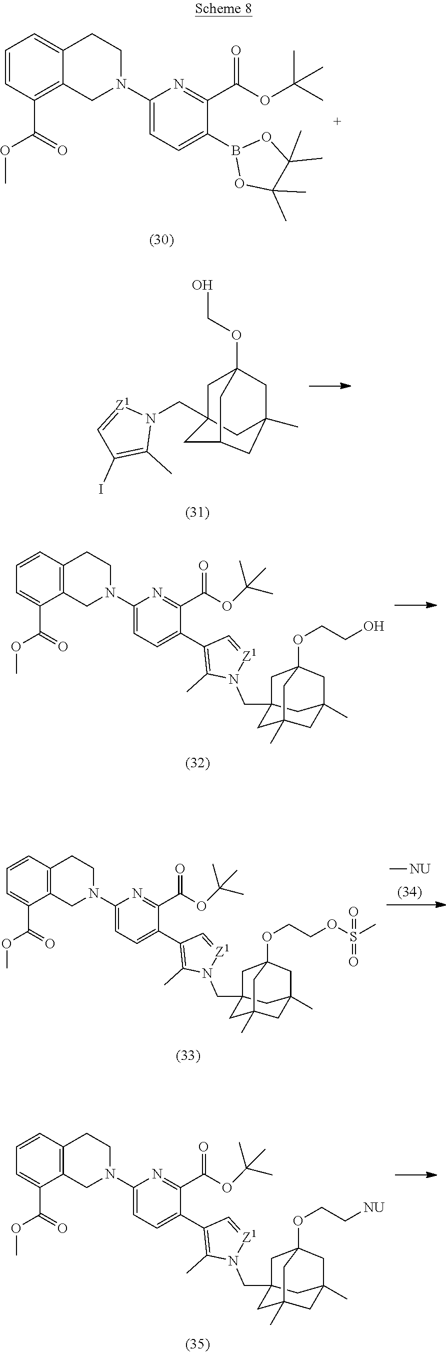

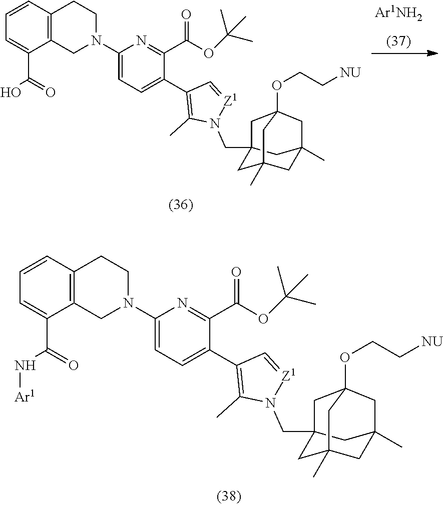

Scheme 8 describes a method to make intermediates which contain -Nu (nucleophile) tethered to an adamantane and picolinate protected as a t-butyl ester. Compound (30) can be reacted with compound (31) under Suzuki Coupling conditions described herein or in the literature to provide methyl compound (32). Compound (32) can be treated with a base such as but not limited to triethylamine, followed by methanesulfonyl chloride to provide compound (33). The addition is typically performed at low temperature before warming up to ambient temperature in a solvent, such as, but not limited to, dichloromethane. Compound (33) can be reacted with a nucleophile (Nu) of formula (34) to provide compound (35). Examples of nucleophiles include, but are not limited to, sodium azide, methylamine, ammonia and di-tert-butyl imidodicarbonate. Compound (17) can be reacted with lithium hydroxide to provide compound (36). The reaction is typically performed at ambient temperature in a solvent such as but not limited to tetrahydrofuran, methanol, water, or mixtures thereof. Compound (36) can be reacted with compound (37) under amidation conditions described herein or readily available in the literature to provide compounds of formula (38).

### 4.9.1.9. Synthesis of Compounds (42) and (43)

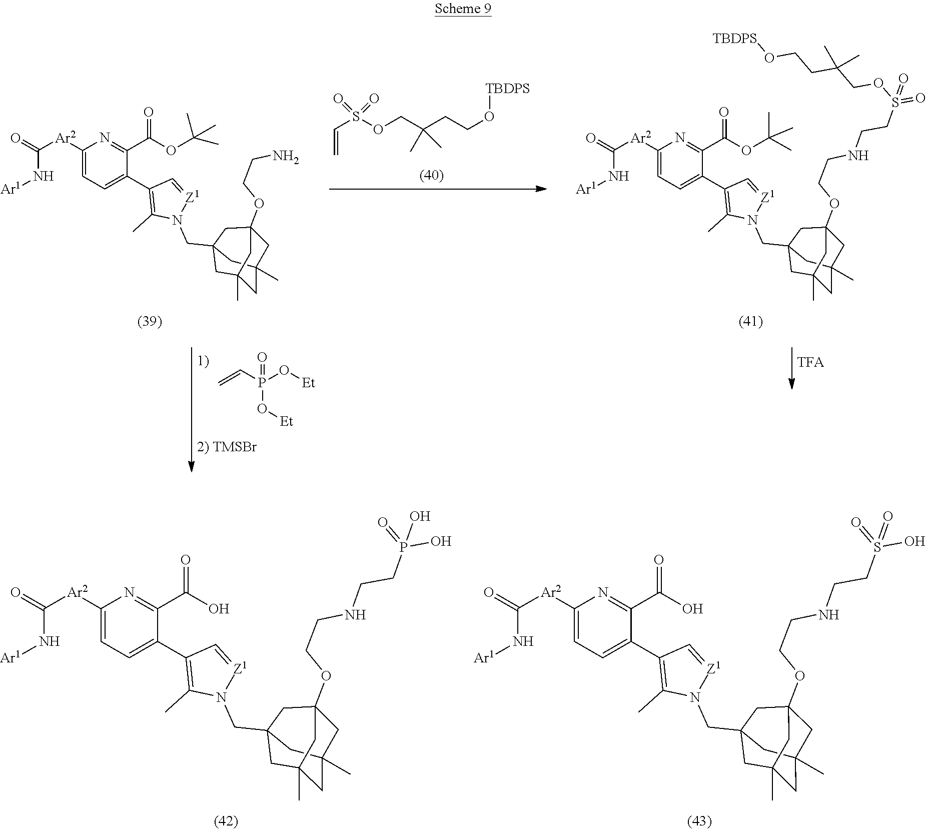

Scheme 9 shows representative methods used to make solubilized Bcl-xL inhibitors. Bcl-xL inhibitors can be synthesized using the general approach of modifying a primary amine with a solubilizing group and then attaching the resulting secondary amine to a linker as described in later schemes. For example, compound (41) can be prepared by reacting compound (39) with compound (40). The reaction is typically performed at ambient temperature in a solvent such as but not limited to N,N-dimethylformamide. Compound (41) can be reacted with trifluoroacetic acid to provide compound (43). The reaction is typically performed at ambient temperature in a solvent such as but not limited to dichloromethane. Another example shown in Scheme 9 is the reaction of compound (39) with diethyl vinylphosphonate, followed by reaction with bromotrimethylsilane and allyltrimethylsilane to provide compound (42). Other examples to introduce solubilizing groups on the Bcl-xL inhibitors described herein include, but are not limited to, reductive amination reactions, alkylations, and amidation reactions.

### 4.9.1.10. Synthesis of Compound (47)

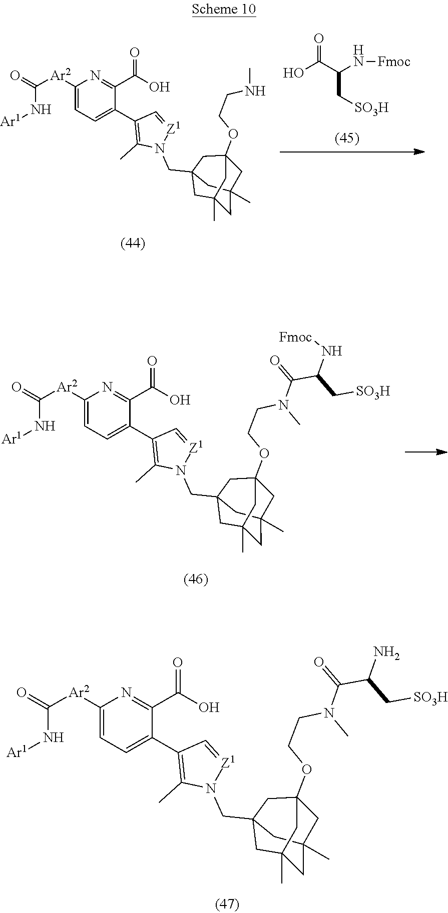

Scheme 10 shows introduction of a solubilizing group by amidation reaction. Bcl-xL inhibitors can be synthesized using the general approach of modifying a primary or secondary amine with a solubilizing group and then attaching the resulting amine to a linker as described in later schemes. For example, compound (45) can be treated sequentially with HATU and compound (44), to provide compound (46). Compound (46) can be treated with diethylamine in solvents such as, but not limited to, N,N-dimethylformamide to give compound (47).

### 4.9.1.11. Synthesis of Compound (51)

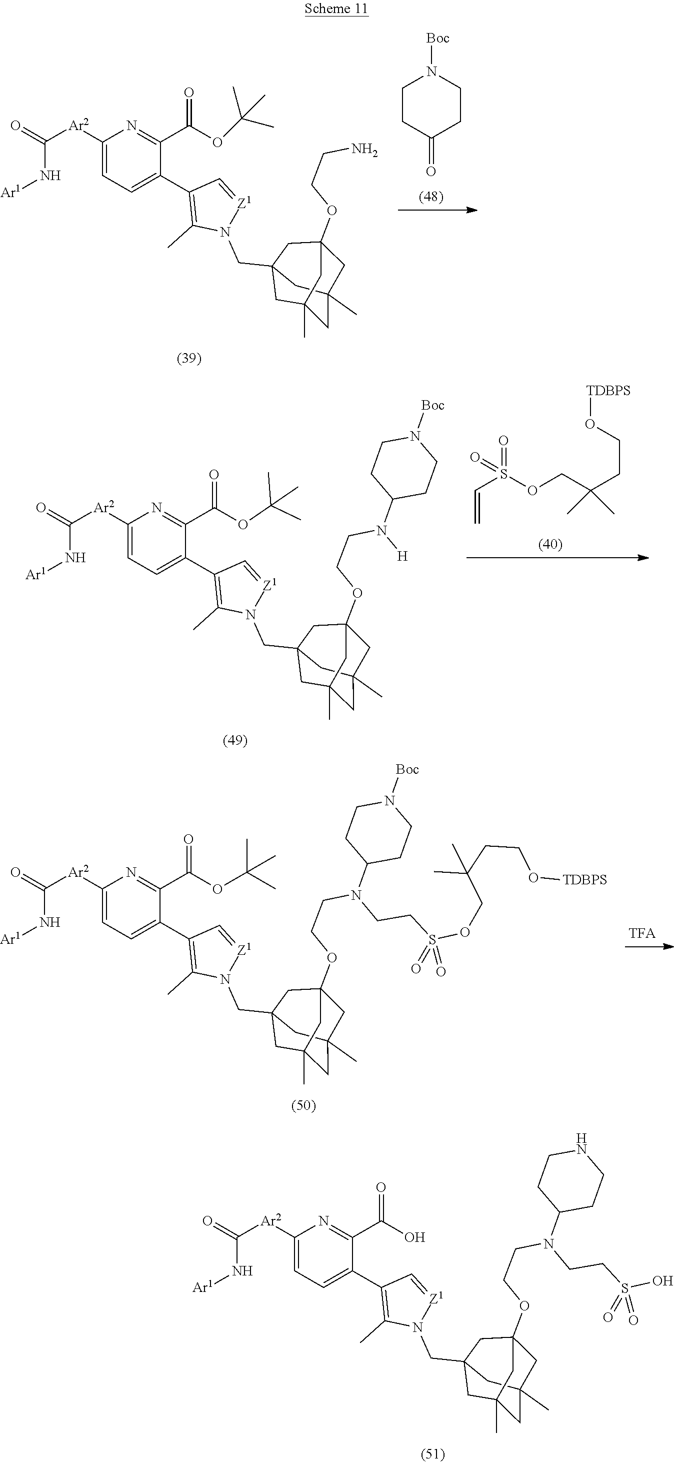

Scheme 11 shows representative methods to make solubilized Bcl-xL inhibitors. Bcl-xL inhibitors can be synthesized using the general approach of modifying a primary amine with a spacer to give a differentially protected diamine. The unprotected secondary amine can be modified with a solubilizing group. Deprotection of a protected amine them reveals a site for linker attachment, as described in later schemes. For example, compound (39) can be reductively alkylated with reagents such as, but not limited to tert-butyl 4-oxopiperidine-1-carboxylate (48), under conditions known in the art, to provide a secondary amine (49). Compound (50) can be prepared by reacting compound (49) with 4-((tert-butyldiphenylsilyl)oxy)-2,2-dimethylbutyl ethanesulfonate (40). The reaction is typically performed at ambient temperature in a solvent such as but not limited to N,N-dimethylformamide. Compound (40) can be reacted with trifluoroacetic acid to provide compound (51). The reaction is typically performed at ambient temperature in a solvent such as but not limited to dichloromethane.

### 4.9.1.12. Synthesis of Compound (61)

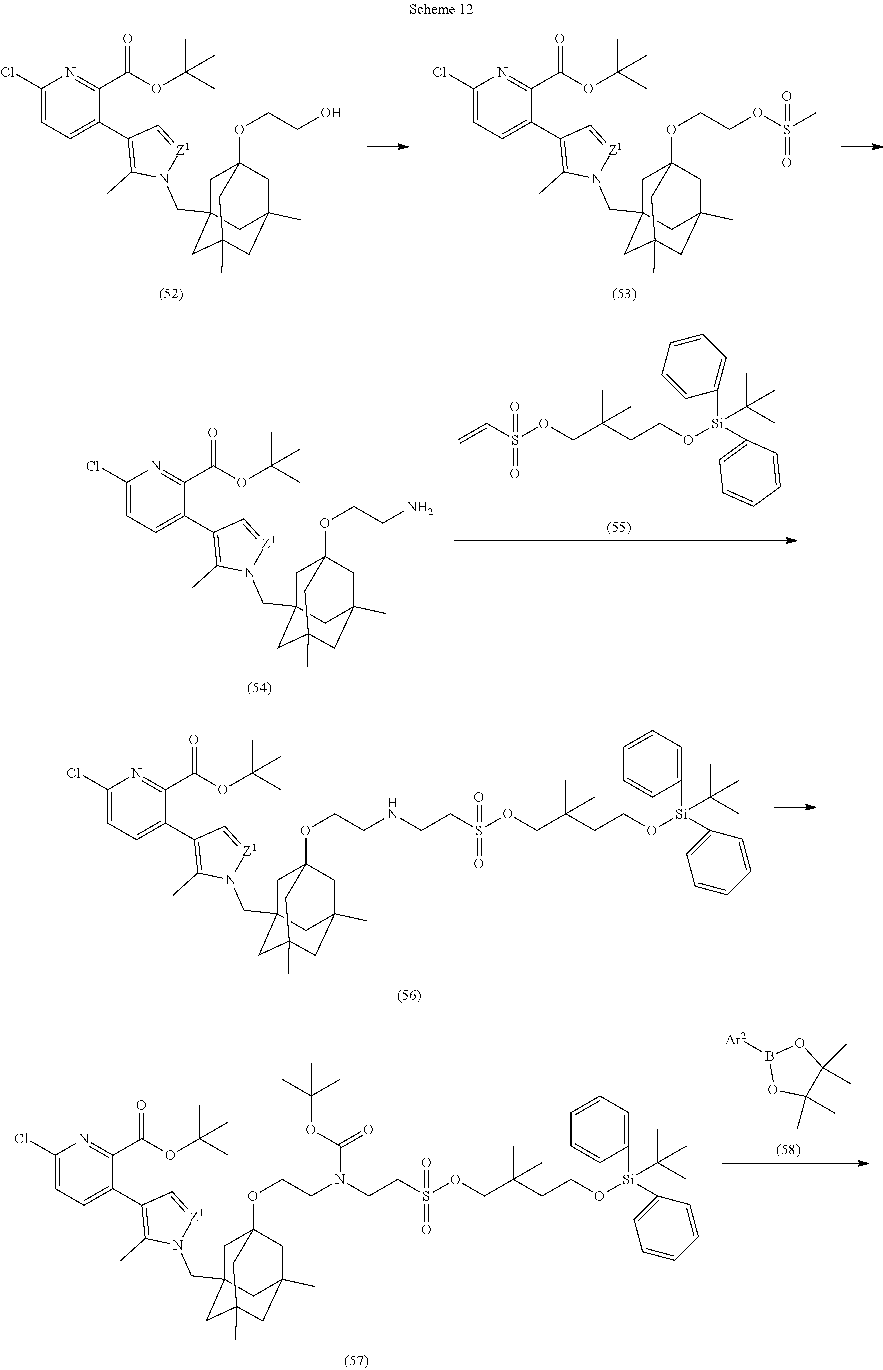

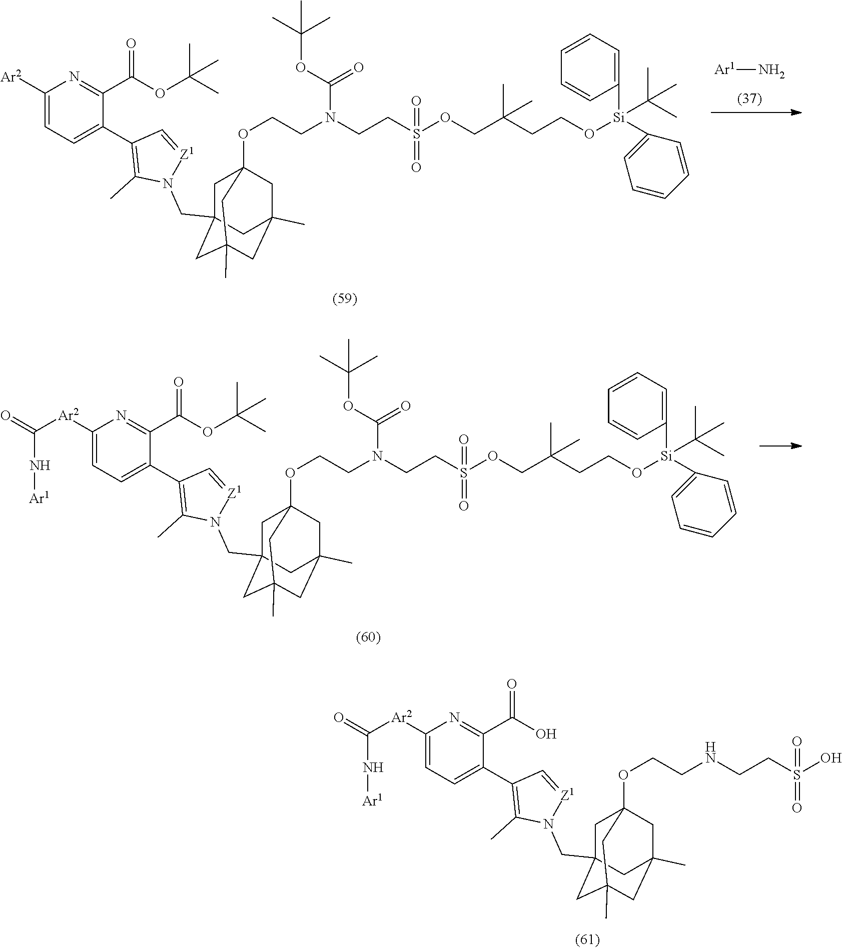

Scheme 12 describes a method to synthesize solubilized Bcl-xL inhibitors. Compound (52) can be reacted with methanesulfonyl chloride, in the presence of a base, such as, but not limited to, triethylamine, to provide compound (53). The reaction is typically performed at a low temperature in a solvent such as but not limited to dichloromethane. Compound (53) can be treated with ammonia in methanol to provide compound (54). The reaction is typically performed at an elevated temperature, and the reaction may be performed under microwave conditions. Compound (56) can be prepared by reacting compound (55) in the presence of a base such as but not limited to N,N-diisopropylethylamine. The reaction is typically performed at ambient temperature in a solvent such as but not limited to N,N-dimethylformamide. Compound (56) can be treated with di-t-butyldicarbonate and 4-(dimethylamino)pyridine to provide compound (57). The reaction is typically performed at ambient temperature in a solvent such as but not limited to tetrahydrofuran. Compound (59) can be prepared by reacting compound (57) with a boronate ester (or the equivalent boronic acid) of formula (58), under Suzuki Coupling conditions described herein or in the literature. Bis(2,5-dioxopyrrolidin-1-yl) carbonate can be reacted with compound (37), followed by reaction with compound (59), to provide compound (60). The reaction is typically performed at ambient temperature in a solvent such as, but not limited to, acetonitrile. Compound (61) can be prepared by treating compound (60) with trifluoroacetic acid. The reaction is typically performed at ambient temperature in a solvent such as but not limited to dichloromethane.

### 4.9.1.13. Synthesis of Compound (70)

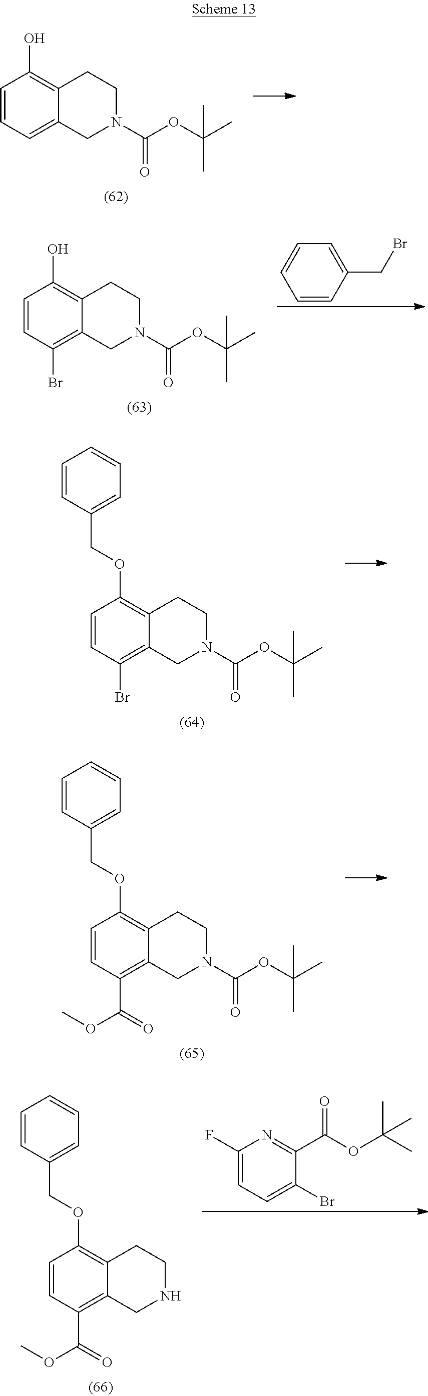

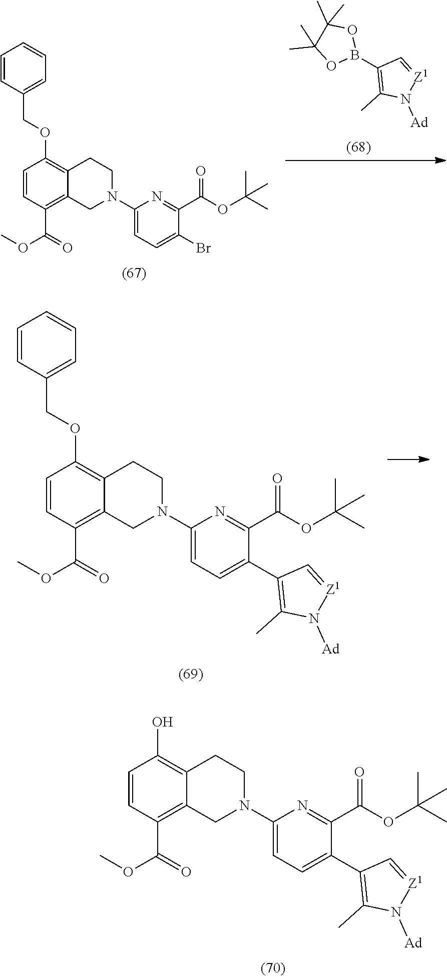

Scheme 13 describes the synthesis of 5-hydroxy tetrahydroisoquinoline intermediates. Compound (63) can be prepared by treating compound (62) with N-bromosuccinimide. The reaction is typically performed at ambient temperature is a solvent such as, but not limited to, N,N-dimethylformamide. Compound (63) can be reacted with benzyl bromide in the presence of a base, such as, but not limited to, potassium carbonate, to provide compound (64). The reaction is typically performed at an elevated temperature, in a solvent such as, but not limited to, acetone. Compound (64) can be treated with carbon monoxide and methanol in the presence of a base, such as, but not limited to, triethylamine, and a catalyst, such as, but not limited to, compound (65). The reaction is typically performed at an elevated temperature under an inert atmosphere. Compound (65) can be treated with an acid, such as, but not limited to, hydrochloric acid in dioxane, to provide compound (66). The reaction is typically performed at ambient temperature in a solvent, such as, but not limited to, tetrahydrofuran. Compound (67) can be prepared by reacting compound (66) with tert-butyl 3-bromo-6-fluoropicolinate in the presence of a base, such as, but not limited to, triethylamine. The reaction is typically performed under an inert atmosphere at an elevated temperature in a solvent, such as, but not limited to, dimethyl sulfoxide. Compound (67) can be reacted with a boronic acid of formula (68), wherein Ad is the methyladamantane moiety of the compounds of the disclosure (e.g., the compounds of formulae (IIa)-(IId)), under Suzuki Coupling conditions described herein or in the literature to provide compound (69). Compound (70) can be prepared by reacting compound (69) with hydrogen in the presence of Pd(OH)₂. The reaction is typically performed at an elevated temperature in a solvent such as, but not limited to tetrahydrofuran.

### 4.9.1.14. Synthesis of Compound (75)

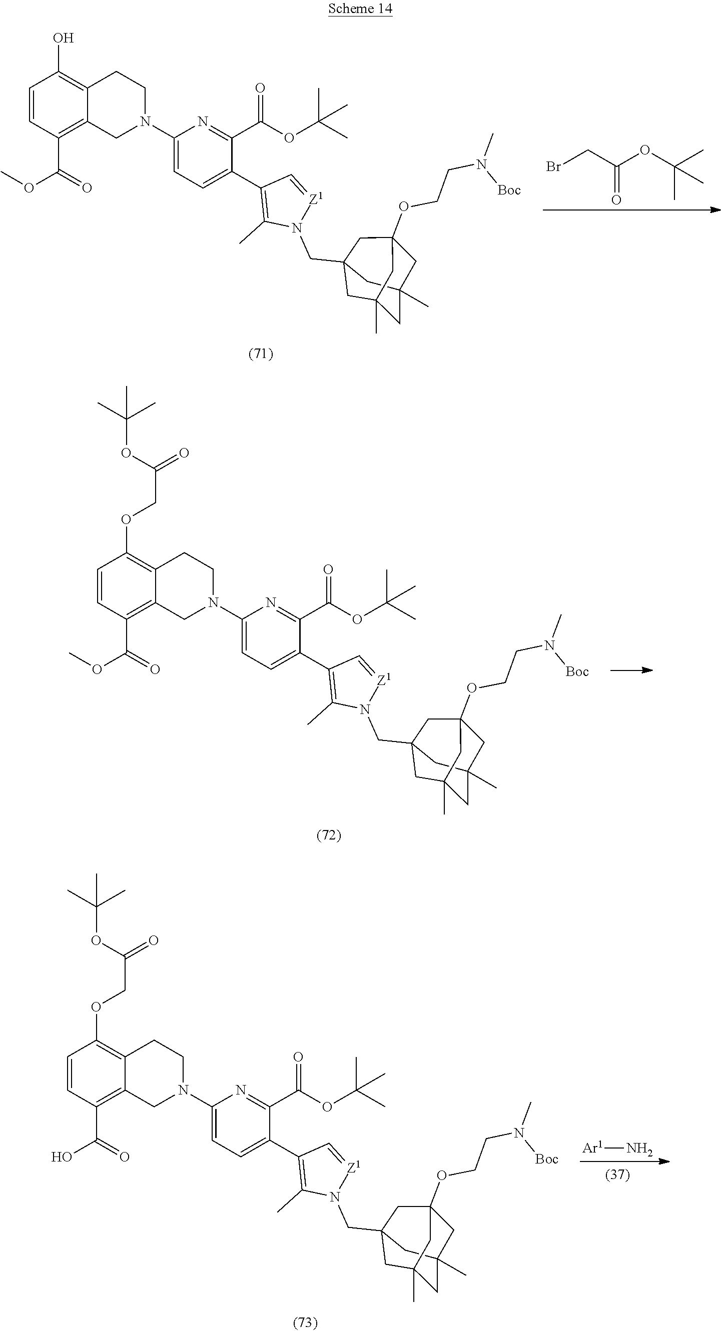

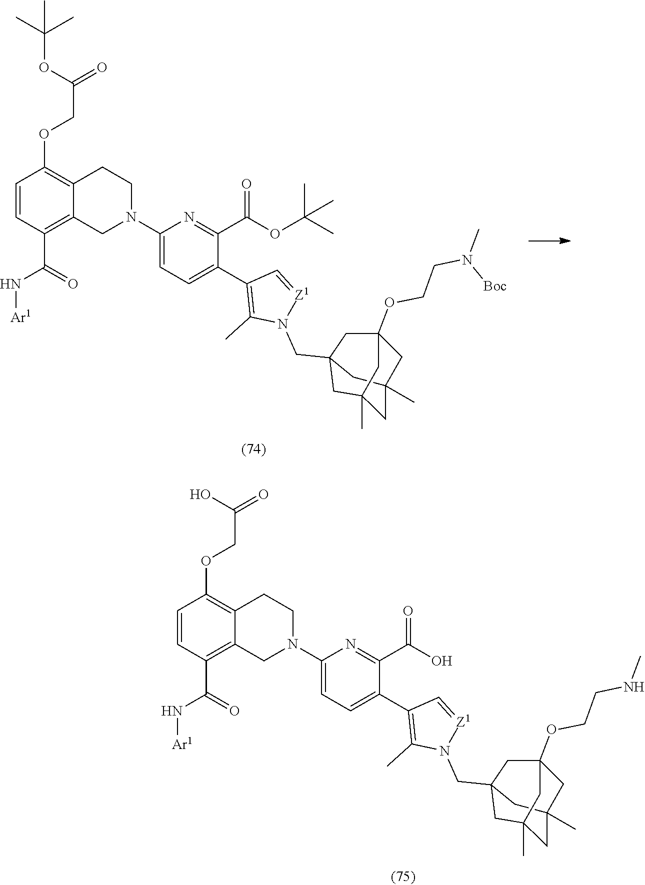

Scheme 14 shows representative methods used to make solubilized Bcl-xL inhibitors. Bcl-xL inhibitors can be synthesized using the general approach of modifying an Ar² substituent with a solubilizing group and then attaching an amine to a linker as described in later schemes. For example, compound (71) can be reacted with tert-butyl 2-bromoacetate in the presence of a base such as, but not limited to, potassium carbonate in a solvent such as, but not limited, to N,N-dimethylformamide. Compound (72) can be treated with aqueous lithium hydroxide in a solvent such as, but not limited to, methanol, tetrahydrofuran or mixtures thereof to provide compound (73). Compound (74) can be obtained by amidation of compound (73) with compound (37) under conditions previously described. Compound (74) can be treated with acids such as, but not limited to trifluoroacetic acid or HCl, to provide a Bcl-xL inhibitor of the formula (75). The reaction is typically performed at ambient temperature in solvents such as, but not limited to, dichloromethane or 1,4-dioxane.

### 4.9.2. General Methods for Synthesizing Synthons

In the schemes below, the various substituents Ar¹, Ar², Z¹, Y, G, R^{11a} and R^{11b} are as defined in the Detailed Description section.

### 4.9.2.1. Synthesis of Compound (89)

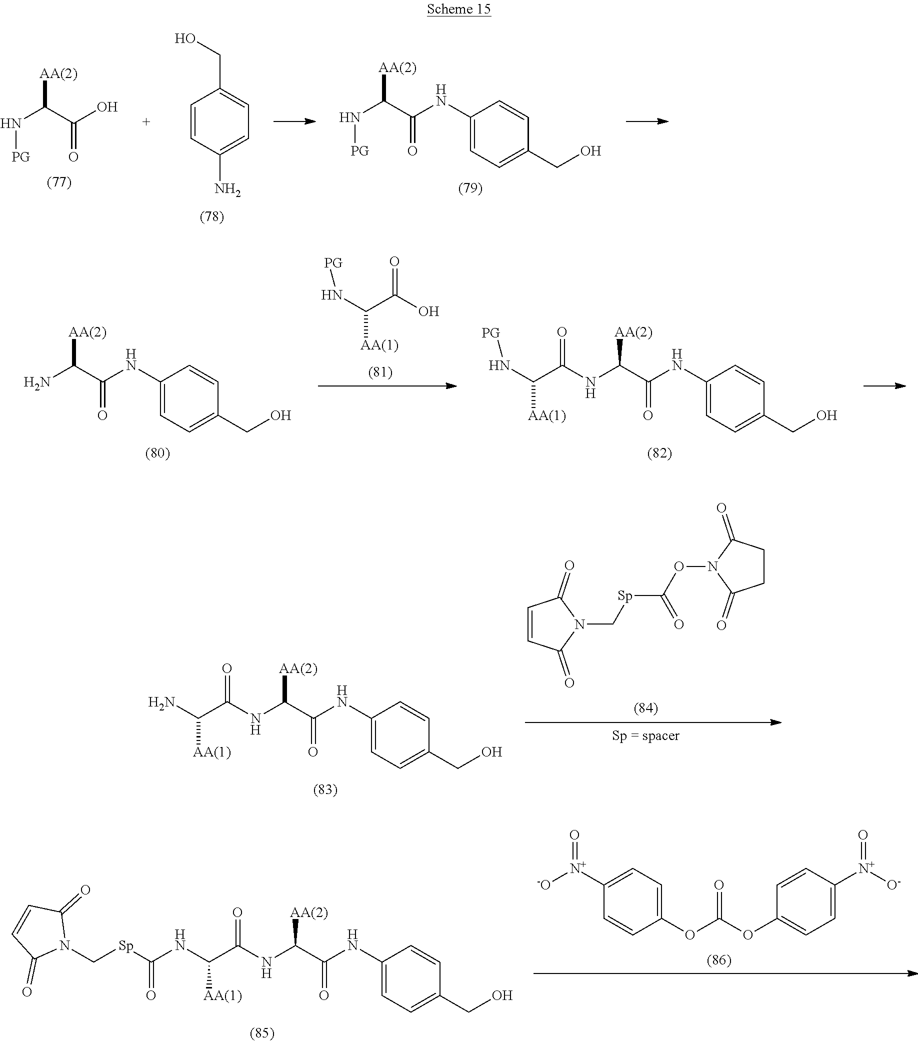

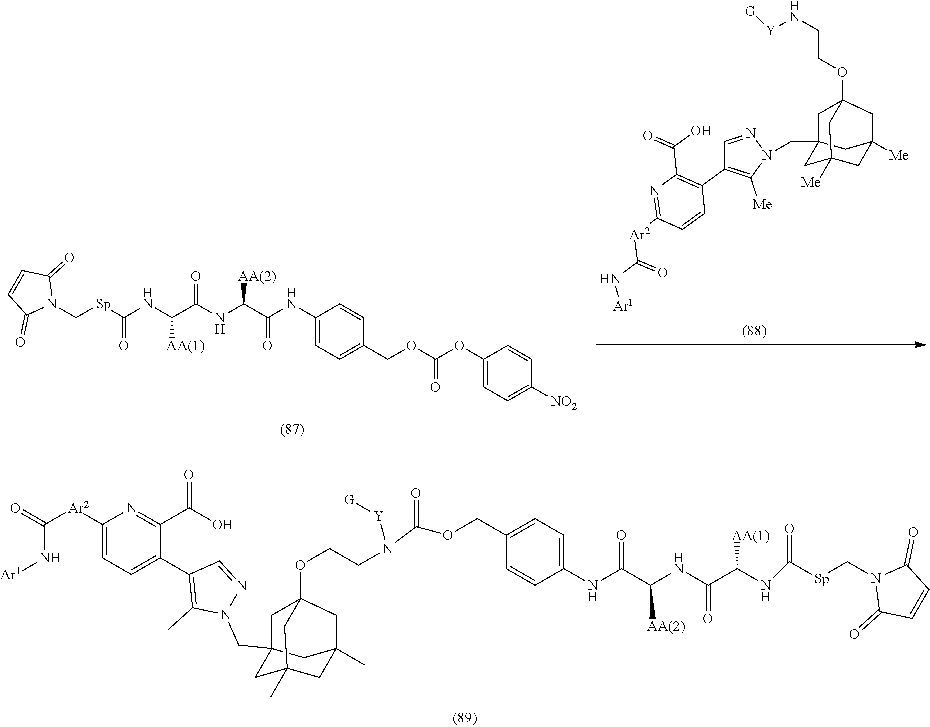

As shown in scheme 15, compounds of formula (77), wherein PG is an appropriate base labile protecting group and AA(2) is Cit, Ala, or Lys, can be reacted with 4-(aminophenyl)methanol (78), under amidation conditions described herein or readily available in the literature to provide compound (79). Compound (80) can be prepared by reacting compound (79) with a base such as, but not limited to, diethylamine. The reaction is typically performed at ambient temperature in a solvent such as but not limited to N,N-dimethylformamide. Compound (81), wherein PG is an appropriate base or acid labile protecting group and AA(1) is Val or Phe, can be reacted with compound (80), under amidation conditions described herein or readily available in the literature to provide compound (82). Compound (83) can be prepared by treating compound (82) with diethylamine or trifluoroacetic acid, as appropriate. The reaction is typically performed at ambient temperature in a solvent such as but not limited to dichloromethane. Compound (84), wherein Sp is a spacer, can be reacted with compound (83) to provide compound (85). The reaction is typically performed at ambient temperature in a solvent such as but not limited to N,N-dimethylformamide. Compound (85) can be reacted with bis(4-nitrophenyl) carbonate (86) in the presence of a base such as, but not limited to N,N-diisopropylethylamine, to provide compounds (87). The reaction is typically performed at ambient temperature in a solvent such as but not limited to N,N-dimethylformamide. Compounds (87) can be reacted with compound (88) in the presence of a base such as, but not limited to, N,N-diisopropylethylamine, to provide compound (89). The reaction is typically performed at ambient temperature in a solvent such as, but not limited to, N,N-dimethylformamide.

### 4.9.2.2. Synthesis of Compounds (94) and (96)

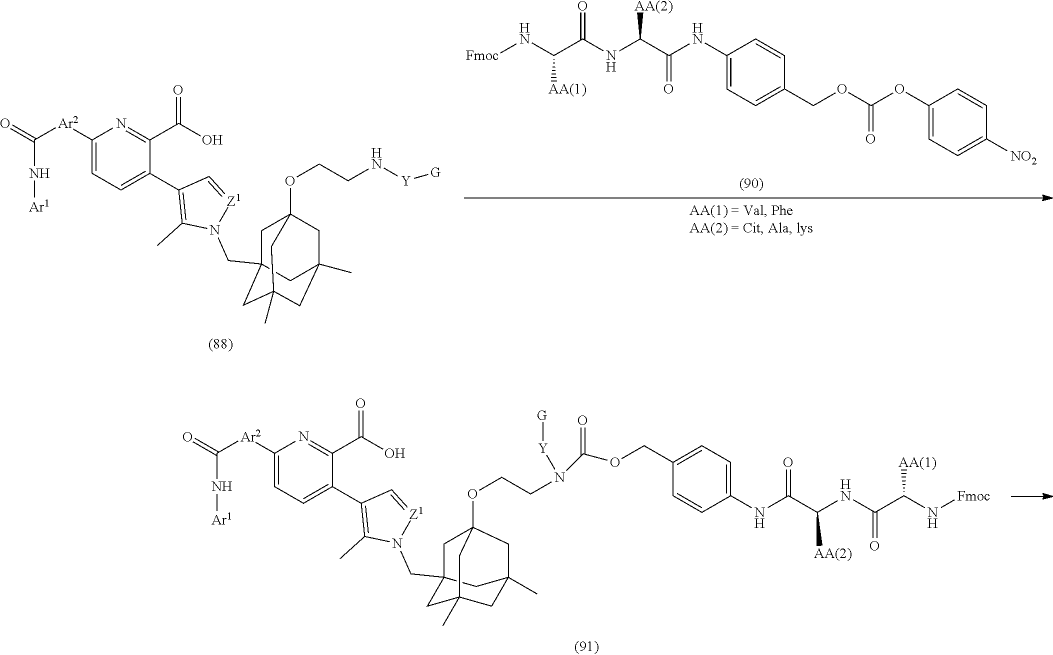

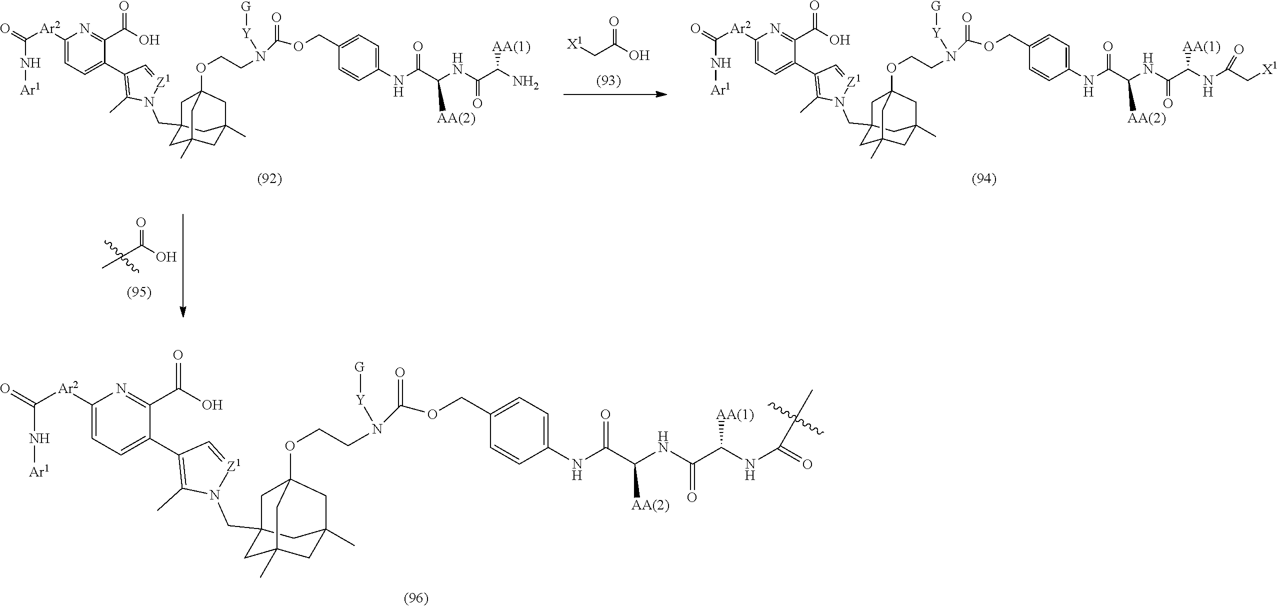

Scheme 16 describes the installment of alternative mAb-linker attachments to dipeptide Synthons. Compound (88) can be reacted with compound (90) in the presence of a base such as, but not limited to, N,N-diisopropylamine to provide compound (91). The reaction is typically performed at ambient temperature in a solvent such as but not limited to N,N-dimethylformamide. Compound (92) can be prepared by reacting compound (91) with diethylamine. The reaction is typically performed at ambient temperature in a solvent such as but not limited to N,N-dimethylformamide. Compound (93), wherein X¹ is Cl, Br, or I, can be reacted with compound (92), under amidation conditions described herein or readily available in the literature to provide compound (94). Compound (92) can be reacted with compounds of formula (95) under amidation conditions described herein or readily available in the literature to provide compound (96).

### 4.9.2.3. Synthesis of Compound (106)

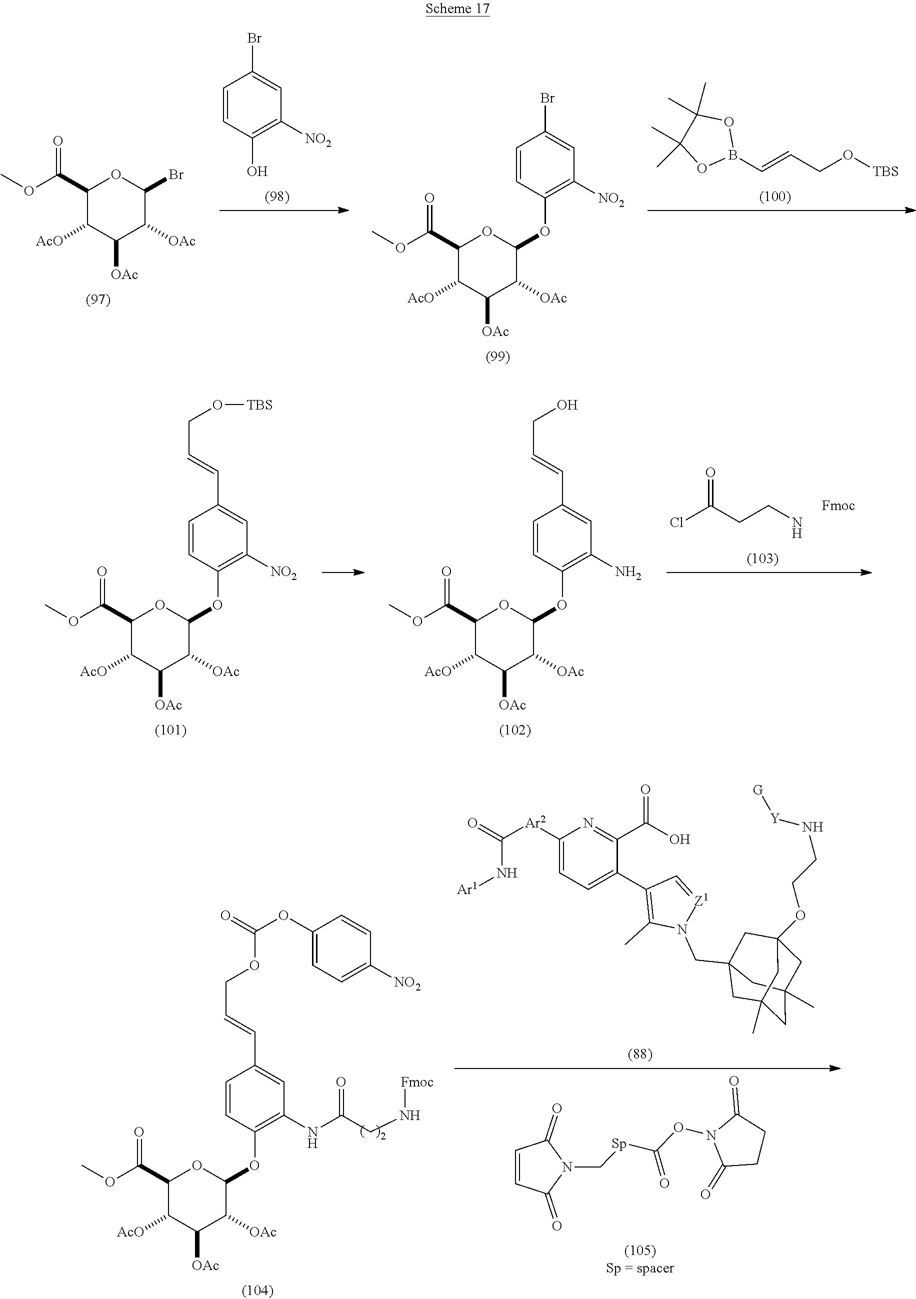

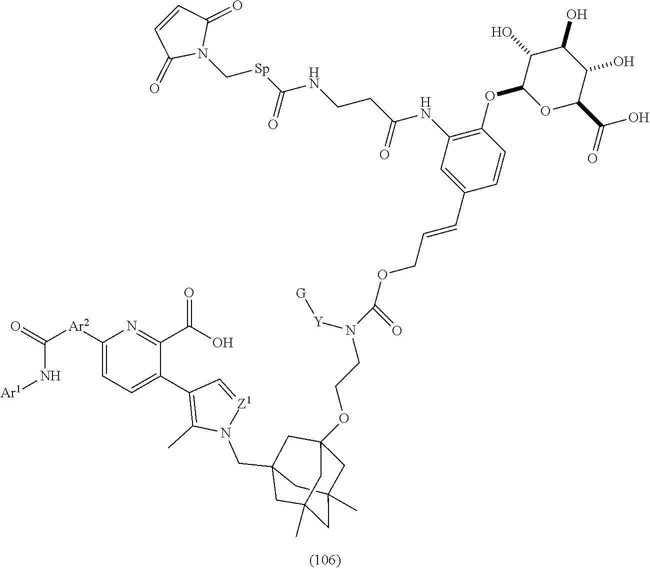

Scheme 17 describes the synthesis of vinyl glucuronide linker intermediates and synthons. (2R,3R,4S,5S,6S)-2-Bromo-6-(methoxycarbonyl)tetrahydro-2H-pyran-3,4,5-triyl triacetate (97) can be treated with silver oxide, followed by 4-bromo-2-nitrophenol (98) to provide (2S,3R,4S,5S,6S)-2-(4-bromo-2-nitrophenoxy)-6-(methoxycarbonyl)tetrahydro-2H-pyran-3,4,5-triyl triacetate (99). The reaction is typically performed at ambient temperature in a solvent, such as, but not limited to, acetonitrile. (2S,3R,4S,5S,6S)-2-(4-Bromo-2-nitrophenoxy)-6-(methoxycarbonyl)tetrahydro-2H-pyran-3,4,5-triyl triacetate (99) can be reacted with (E)-tert-butyldimethyl((3-(4,4,5,5-tetramethyl-1,3,2-dioxaborolan-2-yl)allyl)oxy)silane (100) in the presence of a base such as, but not limited to, sodium carbonate, and a catalyst such as but not limited to tris(dibenzylideneacetone)dipalladium (Pd₂(dba)₃), to provide (2S,3R,4S,5S,6S)-2-(4-((E)-3-((tert-butyldimethylsilyl)oxy)prop-1-en-1-yl)-2-nitrophenoxy)-6-(methoxycarbonyl)tetrahydro-2H-pyran-3,4,5-triyl triacetate (101). The reaction is typically performed at an elevated temperature in a solvent, such as, but not limited to, tetrahydrofuran. (2S,3R,4S,5S,6S)-2-(2-amino-4-((E)-3-hydroxyprop-1-en-1-yl)phenoxy)-6-(methoxycarbonyl)tetrahydro-2H-pyran-3,4,5-triyl triacetate (102) can be prepared by reacting (2S,3R,4S,5S,6S)-2-(4-((E)-3-((tert-butyldimethylsilyl)oxy)prop-1-en-1-yl)-2-nitrophenoxy)-6-(methoxycarbonyl)tetrahydro-2H-pyran-3,4,5-triyl triacetate (101) with zinc in the presence of an acid such as, but not limited to, hydrochloric acid. The addition is typically performed at low temperature before warming to ambient temperature in a solvent such as, but not limited to, tetrahydrofuran, water, or mixtures thereof. (2S,3R,4S,5S,6S)-2-(2-amino-4-((E)-3-hydroxyprop-1-en-1-yl)phenoxy)-6-(methoxycarbonyl)tetrahydro-2H-pyran-3,4,5-triyl triacetate (102) can be reacted with (9H-fluoren-9-yl)methyl (3-chloro-3-oxopropyl)carbamate (103), in the presence of a base such as, but not limited to, N,N-diisopropylethylamine, to provide (2S,3R,4S,5S,6S)-2-(2-(3-((((9H-fluoren-9-yl)methoxy)carbonyl)amino)propanamido)-4-((E)-3-hydroxyprop-1-en-1-yl)phenoxy)-6-(methoxycarbonyl)tetrahydro-2H-pyran-3,4,5-triyl triacetate (104). The addition is typically performed at low temperature before warming to ambient temperature in a solvent such as, but not limited to, dichloromethane. Compound (88) can be reacted with (2S,3R,4S,5S,6S)-2-(2-(3-((((9H-fluoren-9-yl)methoxy)carbonyl)amino)propanamido)-4-((E)-3-hydroxyprop-1-en-1-yl)phenoxy)-6-(methoxycarbonyl)tetrahydro-2H-pyran-3,4,5-triyl triacetate (104) in the presence of a base such as, but not limited to, N-ethyl-N-isopropylpropan-2-amine, followed by work up and reaction with compound (105) in the presence of a base such as, but not limited to, N,N-diisopropylethylamine to provide compound (106). The reactions are typically performed at ambient temperature in a solvent such as, but not limited to N,N-dimethylformamide.

### 4.9.2.4. Synthesis of Compound (115)

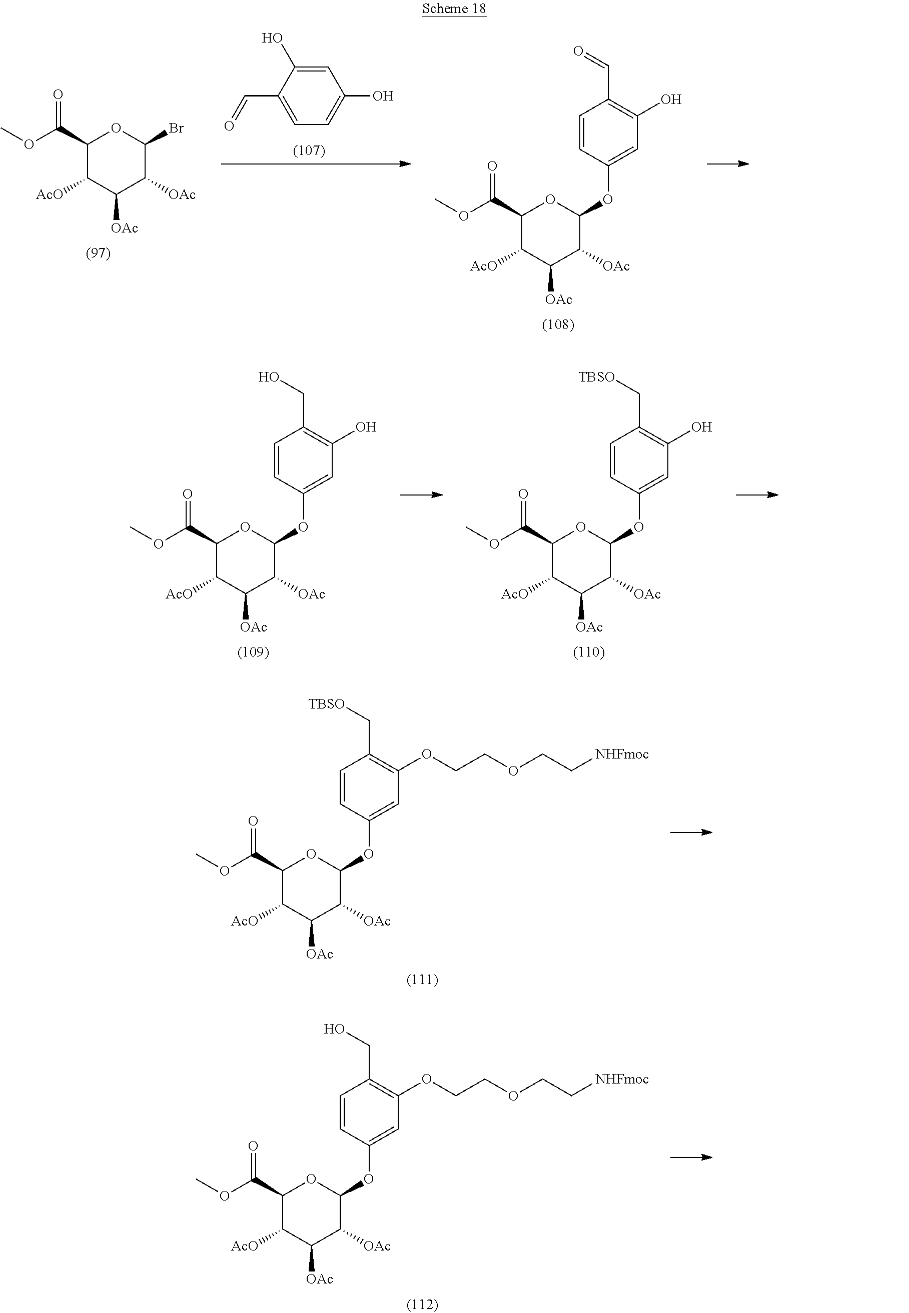

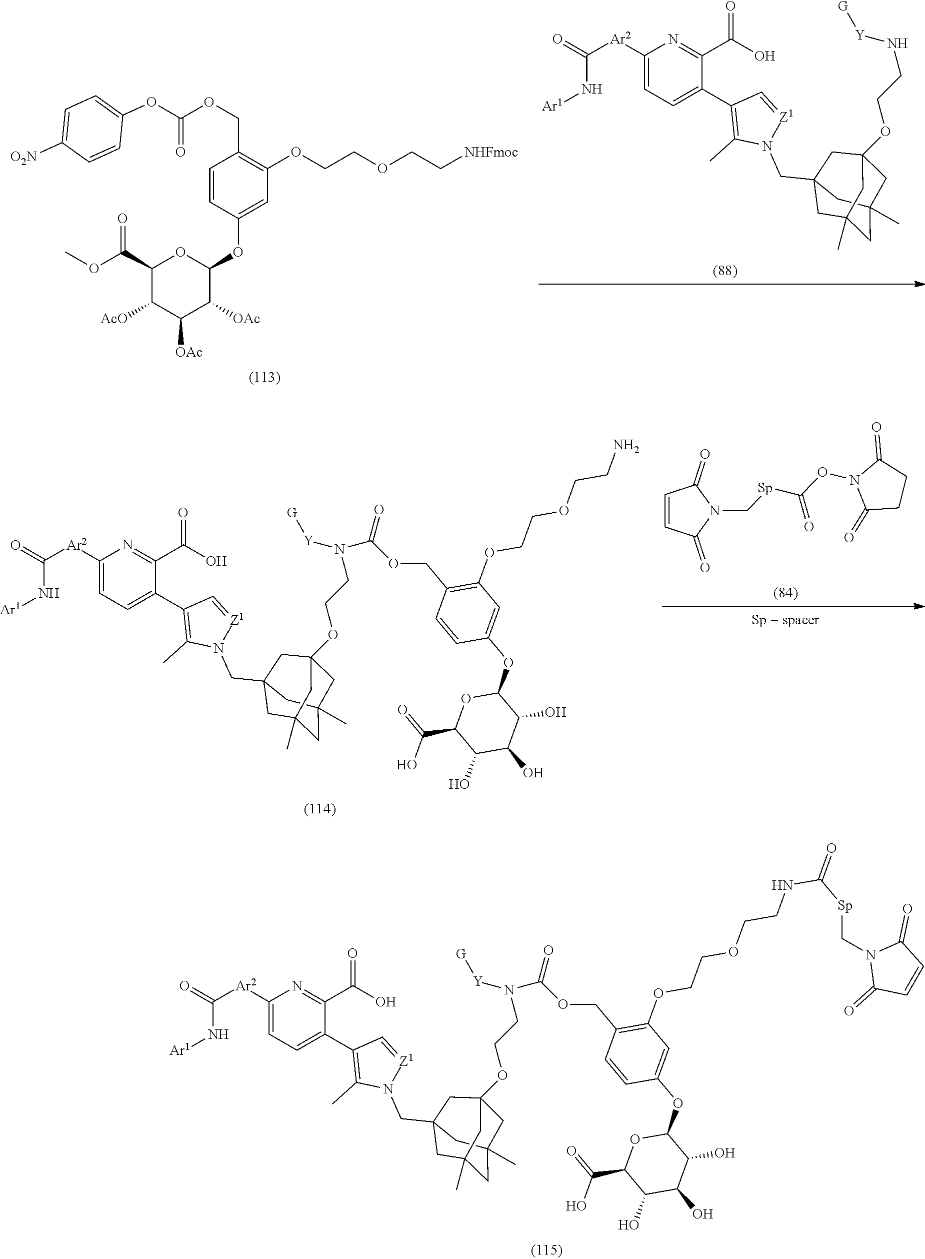

Scheme 18 describes the synthesis of a representative 2-ether glucuronide linker intermediate and synthon. (2S,3R,4S,5S,6S)-2-Bromo-6-(methoxycarbonyl)tetrahydro-2H-pyran-3,4,5-triyl triacetate (97) can be reacted with 2,4-dihydroxybenzaldehyde (107) in the presence of silver carbonate to provide (2S,3R,4S,5S,6S)-2-(4-formyl-3-hydroxyphenoxy)-6-(methoxycarbonyl)tetrahydro-2H-pyran-3,4,5-triyl triacetate (108). The reaction is typically performed at an elevated temperature in a solvent, such as, but not limited to, acetonitrile. (2S,3R,4S,5S,6S)-2-(4-Formyl-3-hydroxyphenoxy)-6-(methoxycarbonyl)tetrahydro-2H-pyran-3,4,5-triyl triacetate (108) can be treated with sodium borohydride to provide (2S,3R,4S,5S,6S)-2-(3-hydroxy-4-(hydroxymethyl)phenoxy)-6-(methoxycarbonyl)tetrahydro-2H-pyran-3,4,5-triyl triacetate (109). The addition is typically performed at low temperature before warming to ambient temperature in a solvent such as but not limited to tetrahydrofuran, methanol, or mixtures thereof. (2S,3R,4S,5S,6S)-2-(4-(((tert-butyldimethylsilyl)oxy)methyl)-3-hydroxyphenoxy)-6-(methoxycarbonyl)tetrahydro-2H-pyran-3,4,5-triyl triacetate (110) can be prepared by reacting (2S,3R,4S,5S,6S)-2-(3-hydroxy-4-(hydroxymethyl)phenoxy)-6-(methoxycarbonyl)tetrahydro-2H-pyran-3,4,5-triyl triacetate (109) with tert-butyldimethylsilyl chloride in the presence of imidazole. The reaction is typically performed at low temperature in a solvent, such as, but not limited to, dichloromethane. (2S,3R,4S,5S,6S)-2-(3-(2-(2-((((9H-Fluoren-9-yl)methoxy)carbonyl)amino)ethoxy)ethoxy)-4-(((tert-butyldimethylsilyl)oxy)methyl)phenoxy)-6-(methoxycarbonyl)tetrahydro-2H-pyran-3,4,5-triyl triacetate (111) can be prepared by reacting (2S,3R,4S,5S,6S)-2-(4-(((tert-butyldimethylsilyl)oxy)methyl)-3-hydroxyphenoxy)-6-(methoxycarbonyl)tetrahydro-2H-pyran-3,4,5-triyl triacetate (110) with (9H-fluoren-9-yl)methyl (2-(2-hydroxyethoxy)ethyl)carbamate in the presence of triphenylphosphine and a azodicarboxylate such as, but not limited to, di-tert-butyl diazene-1,2-dicarboxylate. The reaction is typically performed at ambient temperature in a solvent such as but not limited to toluene. (2S,3R,4S,5S,6S)-2-(3-(2-(2-((((9H-Fluoren-9-yl)methoxy)carbonyl)amino)ethoxy)ethoxy)-4-(((tert-butyldimethylsilyl)oxy)methyl)phenoxy)-6-(methoxycarbonyl)tetrahydro-2H-pyran-3,4,5-triyl triacetate (111) can be treated with acetic acid to provide (2S,3R,4S,5S,6S)-2-(3-(2-(2-((((9H-fluoren-9-yl)methoxy)carbonyl)amino)ethoxy)ethoxy)-4-(hydroxymethyl)phenoxy)-6-(methoxycarbonyl)tetrahydro-2H-pyran-3,4,5-triyl triacetate (112). The reaction is typically performed at ambient temperature in a solvent such as but not limited to water, tetrahydrofuran, or mixtures thereof. (2S,3R,4S,5S,6S)-2-(3-(2-(2-((((9H-Fluoren-9-yl)methoxy)carbonyl)amino)ethoxy)ethoxy)-4-((((4-nitrophenoxy)carbonyl)oxy)methyl)phenoxy)-6-(methoxycarbonyl)tetrahydro-2H-pyran-3,4,5-triyl triacetate (113) can be prepared by reacting (2S,3R,4S,5S,6S)-2-(3-(2-(2-((((9H-fluoren-9-yl)methoxy)carbonyl)amino)ethoxy)ethoxy)-4-(hydroxymethyl)phenoxy)-6-(methoxycarbonyl)tetrahydro-2H-pyran-3,4,5-triyl triacetate (112) with bis(4-nitrophenyl) carbonate in the presence of a base such as but not limited to N-ethyl-N-isopropylpropan-2-amine. The reaction is typically performed at ambient temperature in a solvent such as but not limited to N,N-dimethylformamide. (2S,3R,4S,5S,6S)-2-(3-(2-(2-((((9H-Fluoren-9-yl)methoxy)carbonyl)amino)ethoxy)ethoxy)-4-((((4-nitrophenoxy)carbonyl)oxy)methyl)phenoxy)-6-(methoxycarbonyl)tetrahydro-2H-pyran-3,4,5-triyl triacetate (113) can be treated with compound (88) in the presence of a base such as but not limited to N-ethyl-N-isopropylpropan-2-amine, followed by treatment with lithium hydroxide to provide a compound (114). The reaction is typically performed at ambient temperature in a solvent such as but not limited to N,N-dimethylformamide, tetrahydrofuran, methanol, or mixtures thereof. Compound (115) can be prepared by reacting compound (114) with compound (84) in the presence of a base such as but not limited to N-ethyl-N-isopropylpropan-2-amine. The reaction is typically performed at ambient temperature in a solvent such as but not limited to N,N-dimethylformamide.

### 4.9.2.5. Synthesis of Compound (119)

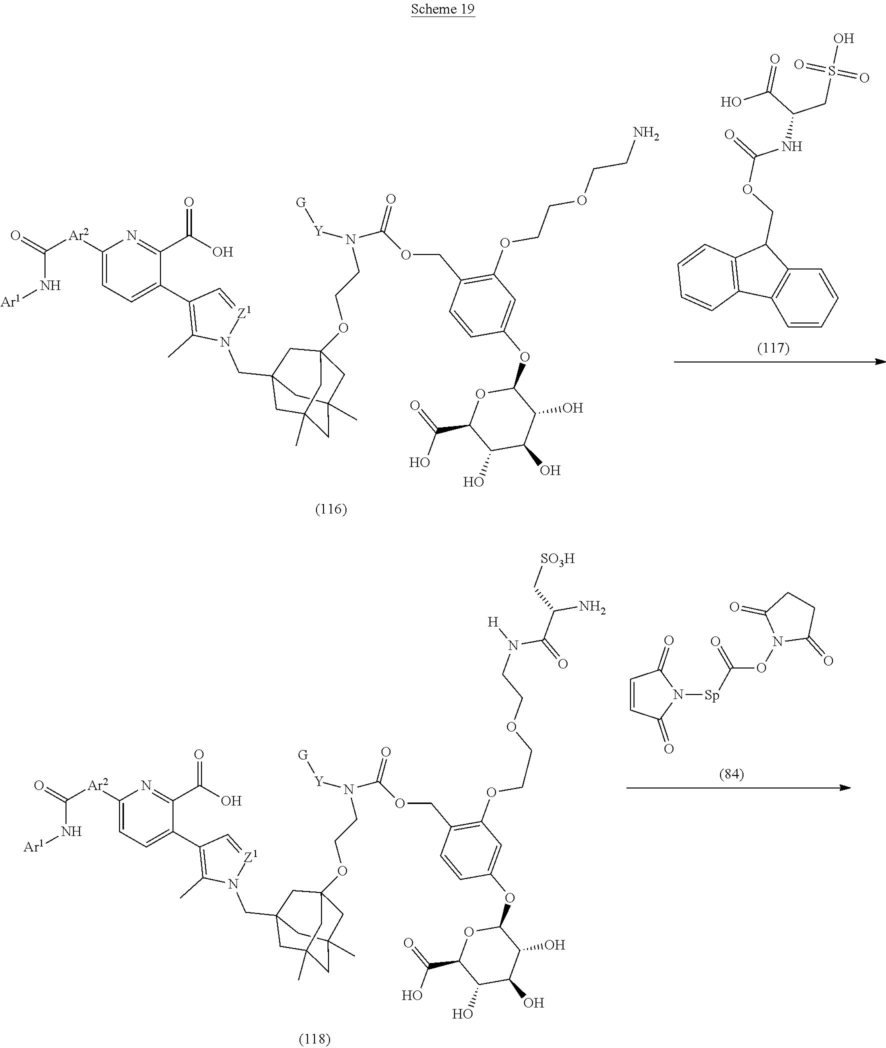

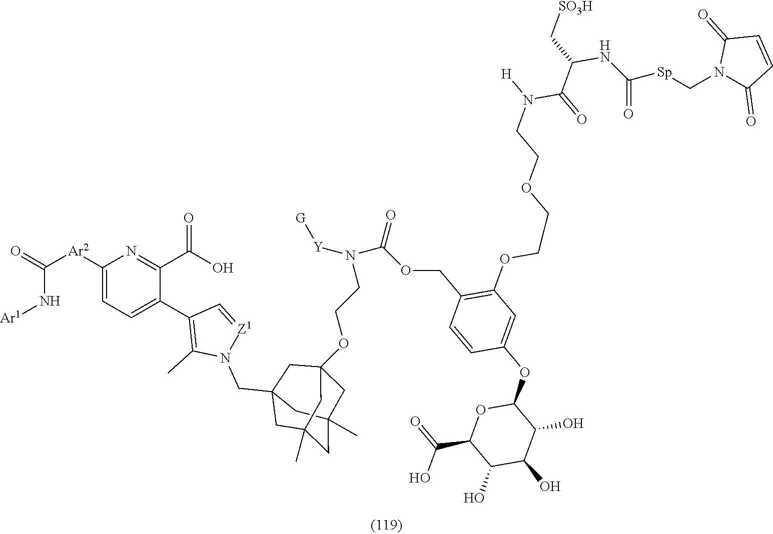

Scheme 19 describes the introduction of a second solubilizing group to a sugar linker. Compound (116) can be reacted with (R)-2-((((9H-fluoren-9-yl)methoxy)carbonyl)amino)-3-sulfopropanoic acid (117), under amidation conditions described herein or readily available in the literature, followed by treatment with a base such as but not limited to diethylamine, to provide compound (118). Compound (118) can be reacted with compound (84), wherein Sp is a spacer, under amidation conditions described herein or readily available in the literature, to provide compound (119).

### 4.9.2.6. Synthesis of Compound (129)

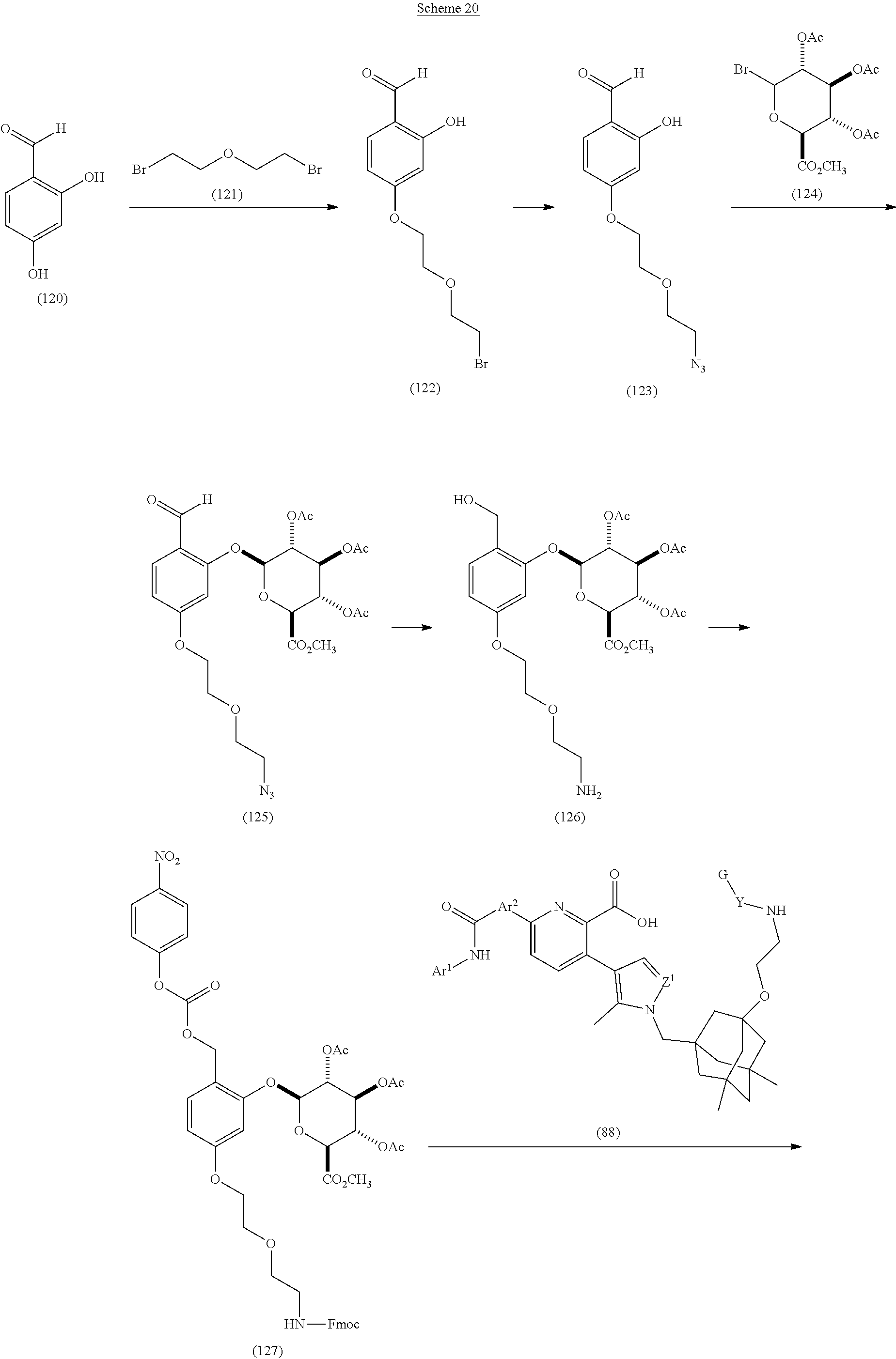

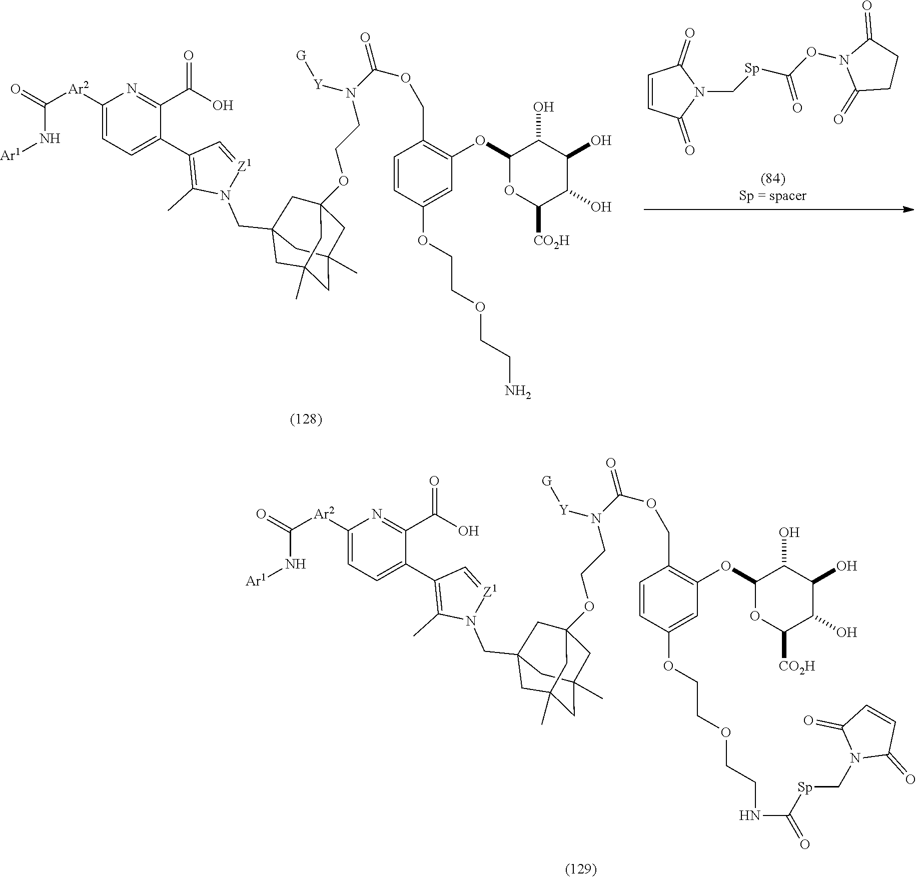

Scheme 20 describes the synthesis of 4-ether glucuronide linker intermediates and synthons. 4-(2-(2-Bromoethoxy)ethoxy)-2-hydroxybenzaldehyde (122) can be prepared by reacting 2,4-dihydroxybenzaldehyde (120) with 1-bromo-2-(2-bromoethoxy)ethane (121) in the presence of a base such as, but not limited to, potassium carbonate. The reaction is typically performed at an elevated temperature in a solvent such as but not limited to acetonitrile. 4-(2-(2-Bromoethoxy)ethoxy)-2-hydroxybenzaldehyde (122) can be treated with sodium azide to provide 4-(2-(2-azidoethoxy)ethoxy)-2-hydroxybenzaldehyde (123). The reaction is typically performed at ambient temperature in a solvent such as but not limited to N,N-dimethylformamide. (2S,3R,4S,5S,6S)-2-(5-(2-(2-Azidoethoxy)ethoxy)-2-formylphenoxy)-6-(methoxycarbonyl)tetrahydro-2H-pyran-3,4,5-triyl triacetate (125) can be prepared by reacting 4-(2-(2-azidoethoxy)ethoxy)-2-hydroxybenzaldehyde (123) with (3R,4S,5S,6S)-2-bromo-6-(methoxycarbonyl)tetrahydro-2H-pyran-3,4,5-triyl triacetate (124) in the presence of silver oxide. The reaction is typically performed at ambient temperature in a solvent such as, but not limited to, acetonitrile. Hydrogenation of (2S,3R,4S,5S,6S)-2-(5-(2-(2-azidoethoxy)ethoxy)-2-formylphenoxy)-6-(methoxycarbonyl)tetrahydro-2H-pyran-3,4,5-triyl triacetate (125) in the presence of Pd/C will provide (2S,3R,4S,5S,6S)-2-(5-(2-(2-aminoethoxy)ethoxy)-2-(hydroxymethyl)phenoxy)-6-(methoxycarbonyl)tetrahydro-2H-pyran-3,4,5-triyl triacetate (126). The reaction is typically performed at ambient temperature in a solvent such as, but not limited to, tetrahydrofuran. (2S,3R,4S,5S,6S)-2-(5-(2-(2-((((9H-Fluoren-9-yl)methoxy)carbonyl)amino)ethoxy)ethoxy)-2-(hydroxymethyl)phenoxy)-6-(methoxycarbonyl)tetrahydro-2H-pyran-3,4,5-triyl triacetate (127) can be prepared by treating (2S,3R,4S,5S,6S)-2-(5-(2-(2-aminoethoxy)ethoxy)-2-(hydroxymethyl)phenoxy)-6-(methoxycarbonyl)tetrahydro-2H-pyran-3,4,5-triyl triacetate (126) with (9H-fluoren-9-yl)methyl carbonochloridate in the presence of a base, such as, but not limited to, N-ethyl-N-isopropylpropan-2-amine. The reaction is typically performed at low temperature in a solvent such as, but not limited to, dichloromethane. Compound (88) can be reacted with (2S,3R,4S,5S,6S)-2-(5-(2-(2-((((9H-Fluoren-9-yl)methoxy)carbonyl)amino)ethoxy)ethoxy)-2-(hydroxymethyl)phenoxy)-6-(methoxycarbonyl)tetrahydro-2H-pyran-3,4,5-triyl triacetate (127) in the presence of a base, such as, but not limited to, N-ethyl-N-isopropylpropan-2-amine, followed by treatment with lithium hydroxide to provide compound (128). The reaction is typically performed at low temperature in a solvent such as, but not limited to, N,N-dimethylformamide. Compound (129) can be prepared by reacting compound (128) with compound (84) in the presence of a base such as, but not limited to, N-ethyl-N-isopropylpropan-2-amine. The reaction is typically performed at ambient temperature in a solvent such as but not limited to N,N-dimethylformamide.

### 4.9.2.7. Synthesis of Compound (139)

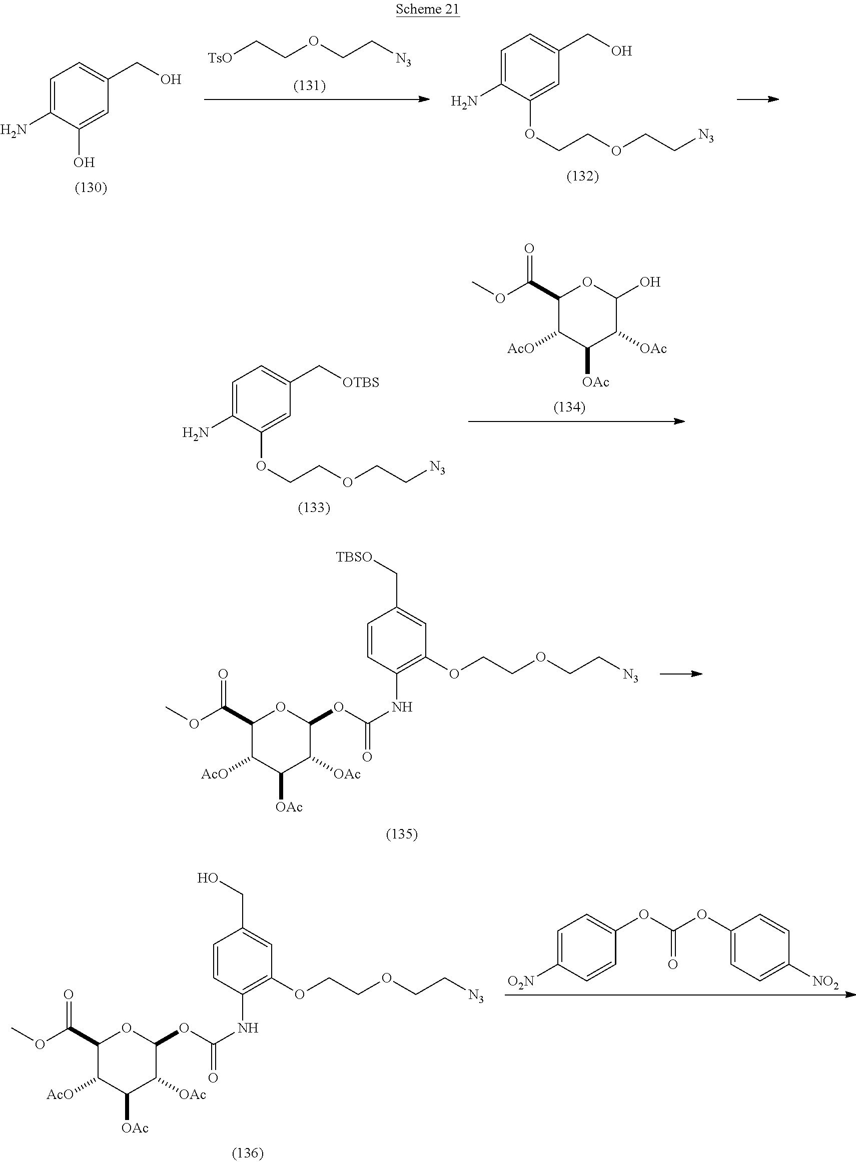

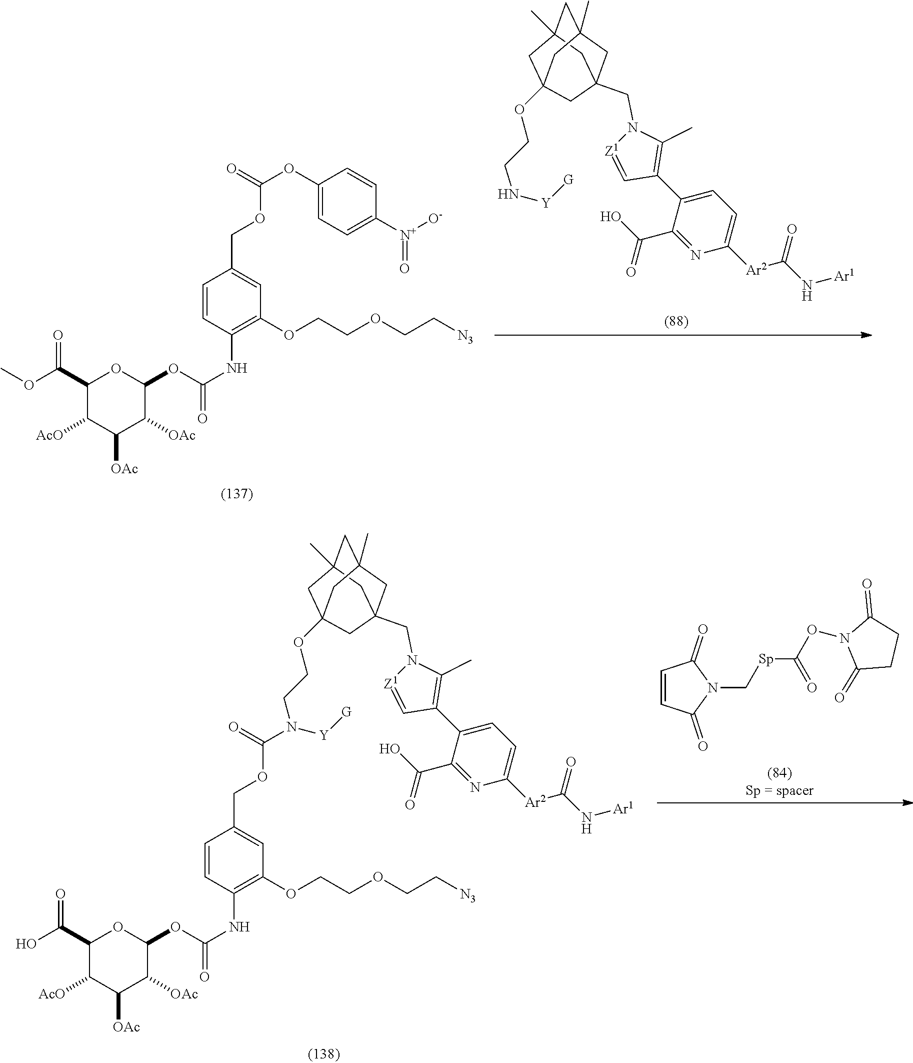

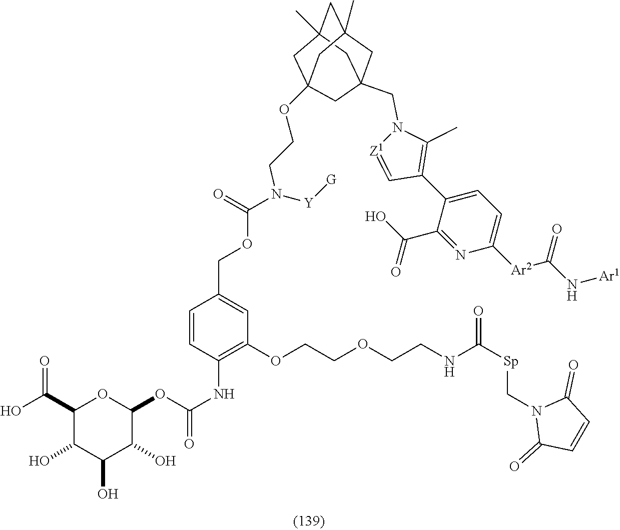

Scheme 21 describes the synthesis of carbamate glucuronide intermediates and synthons. 2-Amino-5-(hydroxymethyl) phenol (130) can be treated with sodium hydride and then reacted with 2-(2-azidoethoxy)ethyl 4-methylbenzenesulfonate (131) to provide (4-amino-3-(2-(2-azidoethoxy)ethoxy)phenyl)methanol (132). The reaction is typically performed at an elevated temperature in a solvent such as, but not limited to N,N-dimethylformamide. 2-(2-(2-Azidoethoxy)ethoxy)-4-(((tert-butyldimethylsilyl)oxy)methyl)aniline (133) can be prepared by reacting (4-amino-3-(2-(2-azidoethoxy)ethoxy)phenyl)methanol (132) with tert-butyldimethylchlorosilane in the presence of imidazole. The reaction is typically performed at ambient temperature in a solvent such as, but not limited to tetrahydrofuran. 2-(2-(2-Azidoethoxy)ethoxy)-4-(((tert-butyldimethylsilyl)oxy)methyl)aniline (133) can be treated with phosgene, in the presence of a base such as but not limited to triethylamine, followed by reaction with (3R,4S,5S,6S)-2-hydroxy-6-(methoxycarbonyl)tetrahydro-2H-pyran-3,4,5-triyl triacetate (134) in the presence of a base such as but not limited to triethylamine, to provide 2S,3R,4S,5S,6S)-2-(((2-(2-(2-azidoethoxy)ethoxy)-4-(((tert-butyldimethylsilyl)oxy)methyl)phenyl)carbamoyl)oxy)-6-(methoxycarbonyl)tetrahydro-2H-pyran-3,4,5-triyl triacetate (135). The reaction is typically performed in a solvent such as, but not limited to, toluene, and the additions are typically performed at low temperature, before warming up to ambient temperature after the phosgene addition and heating at an elevated temperature after the (3R,4S,5S,6S)-2-hydroxy-6-(methoxycarbonyl)tetrahydro-2H-pyran-3,4,5-triyl triacetate (134) addition. (2S,3R,4S,5S,6S)-2-(((2-(2-(2-Azidoethoxy)ethoxy)-4-(hydroxymethyl)phenyl)carbamoyl)oxy)-6-(methoxycarbonyl)tetrahydro-2H-pyran-3,4,5-triyl triacetate (136) can be prepared by reacting 2S,3R,4S,5S,6S)-2-(((2-(2-(2-azidoethoxy)ethoxy)-4-(((tert-butyldimethylsilyl)oxy)methyl)phenyl)carbamoyl)oxy)-6-(methoxycarbonyl)tetrahydro-2H-pyran-3,4,5-triyl triacetate (135) with p-toluenesulfonic acid monohydrate. The reaction is typically performed at ambient temperature in a solvent such as, but not limited to methanol. (2S,3R,4S,5S,6S)-2-(((2-(2-(2-Azidoethoxy)ethoxy)-4-(hydroxymethyl)phenyl)carbamoyl)oxy)-6-(methoxycarbonyl)tetrahydro-2H-pyran-3,4,5-triyl triacetate (136) can be reacted with bis(4-nitrophenyl)carbonate in the presence of a base such as, but not limited to, N,N-diisopropylethylamine, to provide (2S,3R,4S,5S,6S)-2-(((2-(2-(2-azidoethoxy)ethoxy)-4-((((4-nitrophenoxy)carbonyl)oxy)methyl)phenyl)carbamoyl)oxy)-6-(methoxycarbonyl)tetrahydro-2H-pyran-3,4,5-triyl triacetate (137). The reaction is typically performed at ambient temperature in a solvent such as, but not limited to, N,N-dimethylformamide. (2S,3R,4S,5S,6S)-2-(((2-(2-(2-Azidoethoxy)ethoxy)-4-((((4-nitrophenoxy)carbonyl)oxy)methyl)phenyl)carbamoyl)oxy)-6-(methoxycarbonyl)tetrahydro-2H-pyran-3,4,5-triyl triacetate (137) can be reacted with compound in the presence of a base such as, but not limited to, N,N-diisopropylethylamine, followed by treatment with aqueous lithium hydroxide, to provide compound (138). The first step is typically conducted at ambient temperature in a solvent such as, but not limited to N,N-dimethylformamide, and the second step is typically conducted at low temperature in a solvent such as but not limited to methanol. Compound (138) can be treated with tris(2-carboxyethyl))phosphine hydrochloride, followed by reaction with compound (84) in the presence of a base such as, but not limited to, N,N-diisopropylethylamine, to provide compound (139). The reaction with tris(2-carboxyethyl))phosphine hydrochloride is typically performed at ambient temperature in a solvent such as, but not limited to, tetrahydrofuran, water, or mixtures thereof, and the reaction with N-succinimidyl 6-maleimidohexanoate is typically performed at ambient temperature in a solvent such as, but not limited to, N,N-dimethylformamide.

### 4.9.2.8. Synthesis of Compound (149)

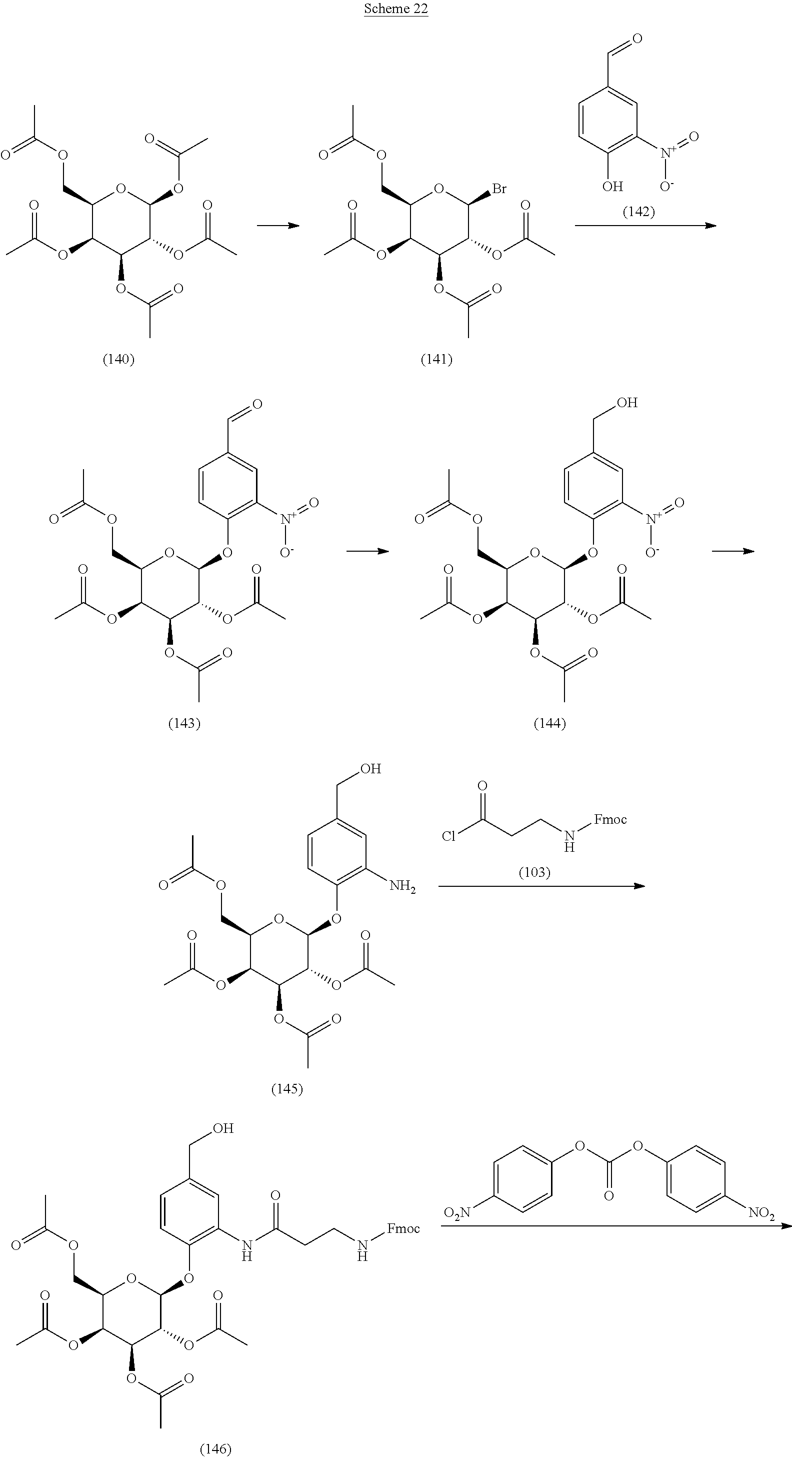

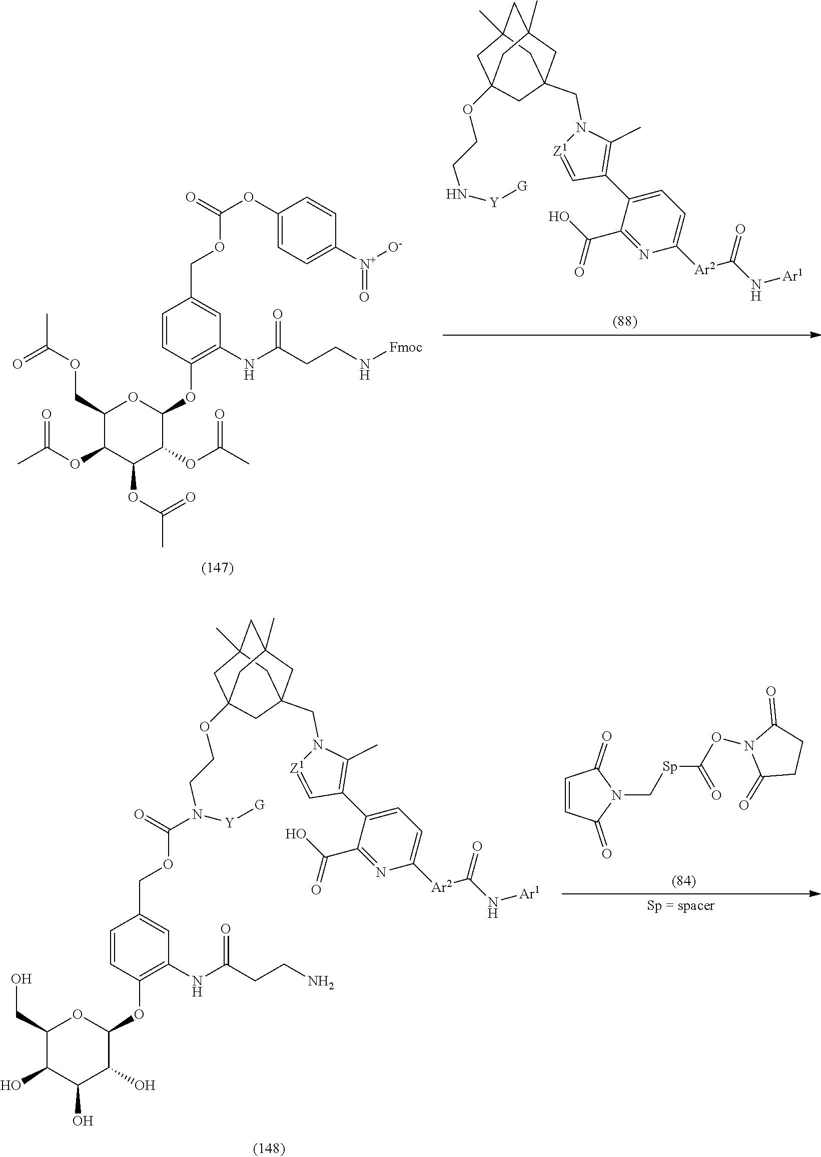

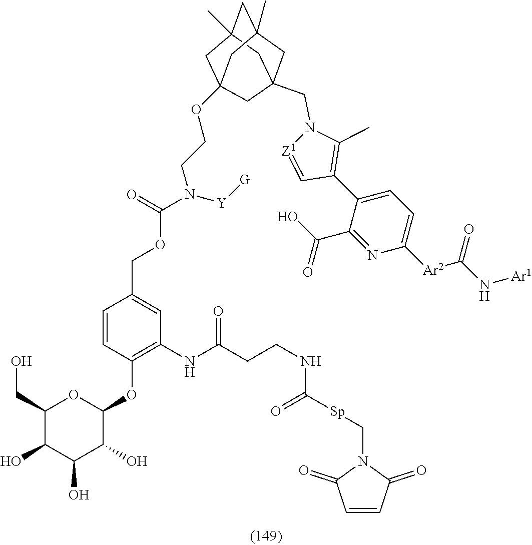

Scheme 22 describes the synthesis of galactoside linker intermediates and synthons. (2S,3R,4S,5S,6R)-6-(Acetoxymethyl)tetrahydro-2H-pyran-2,3,4,5-tetrayl tetraacetate (140) can be treated with HBr in acetic acid to provide (2R,3S,4S,5R,6S)-2-(acetoxymethyl)-6-bromotetrahydro-2H-pyran-3,4,5-triyl triacetate (141). The reaction is typically performed at ambient temperature under a nitrogen atmosphere. (2R,3S,4S,5R,6S)-2-(Acetoxymethyl)-6-(4-formyl-2-nitrophenoxy)tetrahydro-2H-pyran-3,4,5-triyl triacetate (143) can be prepared by treating (2R,3S,4S,5R,6S)-2-(acetoxymethyl)-6-bromotetrahydro-2H-pyran-3,4,5-triyl triacetate (141) with silver(I) oxide in the presence of 4-hydroxy-3-nitrobenzaldehyde (142). The reaction is typically performed at ambient temperature in a solvent such as, but not limited to, acetonitrile. (2R,3S,4S,5R,6S)-2-(Acetoxymethyl)-6-(4-formyl-2-nitrophenoxy)tetrahydro-2H-pyran-3,4,5-triyl triacetate (143) can be treated with sodium borohydride to provide (2R,3S,4S,5R,6S)-2-(acetoxymethyl)-6-(4-(hydroxymethyl)-2-nitrophenoxy)tetrahydro-2H-pyran-3,4,5-triyl triacetate (144). The reaction is typically performed at low temperature in a solvent such as but not limited to tetrahydrofuran, methanol, or mixtures thereof. (2R,3S,4S,5R,6S)-2-(Acetoxymethyl)-6-(2-amino-4-(hydroxymethyl)phenoxy)tetrahydro-2H-pyran-3,4,5-triyl triacetate (145) can be prepared by treating (2R,3S,4S,5R,6S)-2-(acetoxymethyl)-6-(4-(hydroxymethyl)-2-nitrophenoxy)tetrahydro-2H-pyran-3,4,5-triyl triacetate (144) with zinc in the presence of hydrochloric acid. The reaction is typically performed at low temperature, under a nitrogen atmosphere, in a solvent such as, but not limited to, tetrahydrofuran. (2S,3R,4S,5S,6R)-2-(2-(3-((((9H-Fluoren-9-yl)methoxy)carbonyl)amino)propanamido)-4-(hydroxymethyl)phenoxy)-6-(acetoxymethyl)tetrahydro-2H-pyran-3,4,5-triyl triacetate (146) can be prepared by reacting (2R,3S,4S,5R,6S)-2-(acetoxymethyl)-6-(2-amino-4-(hydroxymethyl)phenoxy)tetrahydro-2H-pyran-3,4,5-triyl triacetate (145) with (9H-fluoren-9-yl)methyl (3-chloro-3-oxopropyl)carbamate (103) in the presence of a base such as, but not limited to, N,N-diisopropylethylamine. The reaction is typically performed at low temperature, in a solvent such as, but not limited to, dichloromethane. (2S,3R,4S,5S,6R)-2-(2-(3-((((9H-Fluoren-9-yl)methoxy)carbonyl)amino)propanamido)-4-(hydroxymethyl)phenoxy)-6-(acetoxymethyl)tetrahydro-2H-pyran-3,4,5-triyl triacetate (146) can be reacted with bis(4-nitrophenyl)carbonate in the presence of a base such as, but not limited to, N,N-diisopropylethylamine, to provide (2S,3R,4S,5S,6R)-2-(2-(3-((((9H-fluoren-9-yl)methoxy)carbonyl)amino)propanamido)-4-((((4-nitrophenoxy)carbonyl)oxy)methyl)phenoxy)-6-(acetoxymethyl)tetrahydro-2H-pyran-3,4,5-triyl triacetate (147). The reaction is typically performed at low temperature, in a solvent such as, but not limited to, N,N-dimethylformamide. (2S,3R,4S,5S,6R)-2-(2-(3-((((9H-Fluoren-9-yl)methoxy)carbonyl)amino)propanamido)-4-((((4-nitrophenoxy)carbonyl)oxy)methyl)phenoxy)-6-(acetoxymethyl)tetrahydro-2H-pyran-3,4,5-triyl triacetate (147) can be reacted with compound (88) in the presence of a base such as, but not limited to N,N-diisopropylethylamine, followed by treatment with lithium hydroxide, to provide compound (148). The first step is typically performed at low temperature, in a solvent such as, but not limited to, N,N-dimethylformamide, and the second step is typically performed at ambient temperature, in a solvent such as, but not limited to, methanol. Compound (148) can be treated with compound (84), wherein Sp is a spacer, in the presence of a base, such as, but not limited to N,N-diisopropylethylamine, to provide compound (149). The reaction is typically performed at ambient temperature, in a solvent such as, but not limited to, N,N-dimethylformamide.

### 4.10. Compositions

The Bcl-xL inhibitors and/or ADCs described herein may be in the form of compositions comprising the inhibitor or ADC and one or more carriers, excipients and/or diluents. The compositions may be formulated for specific uses, such as for veterinary uses or pharmaceutical uses in humans. The form of the composition (e.g., dry powder, liquid formulation, etc.) and the excipients, diluents and/or carriers used will depend upon the intended uses of the inhibitors and/or ADCs and, for therapeutic uses, the mode of administration.

For therapeutic uses, the Bcl-xL inhibitor and/or ADC compositions may be supplied as part of a sterile, pharmaceutical composition that includes a pharmaceutically acceptable carrier. This composition can be in any suitable form (depending upon the desired method of administering it to a patient). The pharmaceutical composition can be administered to a patient by a variety of routes such as orally, transdermally, subcutaneously, intranasally, intravenously, intramuscularly, intrathecally, topically or locally. The most suitable route for administration in any given case will depend on the particular Bcl-xL inhibitor or ADC, the subject, and the nature and severity of the disease and the physical condition of the subject. Typically, the Bcl-xL inhibitors will be administered orally or parenterally, and ADC pharmaceutical composition will be administered intravenously or subcutaneously.

Pharmaceutical compositions can be conveniently presented in unit dosage forms containing a predetermined amount of Bcl-xL inhibitor or an ADC described herein per dose. The quantity of inhibitor or ADC included in a unit dose will depend on the disease being treated, as well as other factors as are well known in the art. For Bcl-xL inhibitors, such unit dosages may be in the form of tablets, capsules, lozenges, etc. containing an amount of Bcl-xL inhibitor suitable for a single administration. For ADCs, such unit dosages may be in the form of a lyophilized dry powder containing an amount of ADC suitable for a single administration, or in the form of a liquid. Dry powder unit dosage forms may be packaged in a kit with a syringe, a suitable quantity of diluent and/or other components useful for administration. Unit dosages in liquid form may be conveniently supplied in the form of a syringe pre-filled with a quantity of ADC suitable for a single administration.

The pharmaceutical compositions may also be supplied in bulk from containing quantities of ADC suitable for multiple administrations

Pharmaceutical compositions of ADCs may be prepared for storage as lyophilized formulations or aqueous solutions by mixing an ADC having the desired degree of purity with optional pharmaceutically-acceptable carriers, excipients or stabilizers typically employed in the art (all of which are referred to herein as “carriers”), i.e., buffering agents, stabilizing agents, preservatives, isotonifiers, non-ionic detergents, antioxidants, and other miscellaneous additives. See, Remington's Pharmaceutical Sciences, 16th edition (Osol, ed. 1980). Such additives should be nontoxic to the recipients at the dosages and concentrations employed.

Buffering agents help to maintain the pH in the range which approximates physiological conditions. They may be present at concentrations ranging from about 2 mM to about 50 mM. Suitable buffering agents for use with the present disclosure include both organic and inorganic acids and salts thereof such as citrate buffers (e.g., monosodium citrate-disodium citrate mixture, citric acid-trisodium citrate mixture, citric acid-monosodium citrate mixture, etc.), succinate buffers (e.g., succinic acid-monosodium succinate mixture, succinic acid-sodium hydroxide mixture, succinic acid-disodium succinate mixture, etc.), tartrate buffers (e.g., tartaric acid-sodium tartrate mixture, tartaric acid-potassium tartrate mixture, tartaric acid-sodium hydroxide mixture, etc.), fumarate buffers (e.g., fumaric acid-monosodium fumarate mixture, fumaric acid-disodium fumarate mixture, monosodium fumarate-disodium fumarate mixture, etc.), gluconate buffers (e.g., gluconic acid-sodium gluconate mixture, gluconic acid-sodium hydroxide mixture, gluconic acid-potassium gluconate mixture, etc.), oxalate buffer (e.g., oxalic acid-sodium oxalate mixture, oxalic acid-sodium hydroxide mixture, oxalic acid-potassium oxalate mixture, etc.), lactate buffers (e.g., lactic acid-sodium lactate mixture, lactic acid-sodium hydroxide mixture, lactic acid-potassium lactate mixture, etc.) and acetate buffers (e.g., acetic acid-sodium acetate mixture, acetic acid-sodium hydroxide mixture, etc.). Additionally, phosphate buffers, histidine buffers and trimethylamine salts such as Tris can be used.

Preservatives may be added to retard microbial growth, and can be added in amounts ranging from about 0.2%-1% (w/v). Suitable preservatives for use with the present disclosure include phenol, benzyl alcohol, meta-cresol, methyl paraben, propyl paraben, octadecyldimethylbenzyl ammonium chloride, benzalconium halides (e.g., chloride, bromide, and iodide), hexamethonium chloride, and alkyl parabens such as methyl or propyl paraben, catechol, resorcinol, cyclohexanol, and 3-pentanol. Isotonicifiers sometimes known as “stabilizers” can be added to ensure isotonicity of liquid compositions of the present disclosure and include polyhydric sugar alcohols, for example trihydric or higher sugar alcohols, such as glycerin, erythritol, arabitol, xylitol, sorbitol and mannitol. Stabilizers refer to a broad category of excipients which can range in function from a bulking agent to an additive which solubilizes the therapeutic agent or helps to prevent denaturation or adherence to the container wall. Typical stabilizers can be polyhydric sugar alcohols (enumerated above); amino acids such as arginine, lysine, glycine, glutamine, asparagine, histidine, alanine, ornithine, L-leucine, 2-phenylalanine, glutamic acid, threonine, etc., organic sugars or sugar alcohols, such as lactose, trehalose, stachyose, mannitol, sorbitol, xylitol, ribitol, myoinisitol, galactitol, glycerol and the like, including cyclitols such as inositol; polyethylene glycol; amino acid polymers; sulfur containing reducing agents, such as urea, glutathione, thioctic acid, sodium thioglycolate, thioglycerol, α-monothioglycerol and sodium thio sulfate; low molecular weight polypeptides (e.g., peptides of 10 residues or fewer); proteins such as human serum albumin, bovine serum albumin, gelatin or immunoglobulins; hydrophylic polymers, such as polyvinylpyrrolidone monosaccharides, such as xylose, mannose, fructose, glucose; disaccharides such as lactose, maltose, sucrose and trisaccacharides such as raffinose; and polysaccharides such as dextran.

Non-ionic surfactants or detergents (also known as “wetting agents”) may be added to help solubilize the glycoprotein as well as to protect the glycoprotein against agitation-induced aggregation, which also permits the formulation to be exposed to shear surface stressed without causing denaturation of the protein. Suitable non-ionic surfactants include polysorbates (20, 80, etc.), polyoxamers (184, 188 etc.), Pluronic polyols, polyoxyethylene sorbitan monoethers (TWEEN®-20, TWEEN®-80, etc.). Non-ionic surfactants may be present in a range of about 0.05 mg/ml to about 1.0 mg/ml, for example about 0.07 mg/ml to about 0.2 mg/ml.

Additional miscellaneous excipients include bulking agents (e.g., starch), chelating agents (e.g., EDTA), antioxidants (e.g., ascorbic acid, methionine, vitamin E), and cosolvents.

### 4.11. Methods of Use

The Bcl-xL inhibitors included in the ADCs, as well as the synthons delivered by the ADCs, inhibit Bcl-xL activity and induce apoptosis in cells expressing Bcl-xL. Accordingly, the Bcl-xL inhibitors and/or ADCs may be used in methods to inhibit Bcl-xL activity and/or induce apoptosis in cells.

For Bcl-xL inhibitors, the method generally involves contacting a cell whose survival depends, at least in part, upon Bcl-xL expression with an amount of a Bcl-xL inhibitor sufficient to inhibit Bcl-xL activity and/or induce apoptosis. For ADCs, the method generally involves contacting a cell whose survival depends, at least in part upon Bcl-xL expression, and that expresses a cell-surface antigen for the antibody of the ADC with an ADC under conditions in which the ADC binds the antigen.

In certain embodiments, especially those in which the Bcl-xL inhibitor that comprises the ADC has low or very low cell permeability, the antibody of the ADC binds a target capable of internalizing the ADC into the cell, where it can deliver its Bcl-xL inhibitory synthon. The method may be carried out in vitro in a cellular assay to inhibit Bcl-xL activity and/or inhibit apoptosis, or in vivo as a therapeutic approach towards treating diseases in which inhibition of apoptosis and/or induction of apoptosis would be desirable.

Dysregulated apoptosis has been implicated in a variety of diseases, including, for example, autoimmune disorders (e.g., systemic lupus erythematosus, rheumatoid arthritis, graft-versus-host disease, myasthenia gravis, or Sjogren's syndrome), chronic inflammatory conditions (e.g., psoriasis, asthma or Crohn's disease), hyperproliferative disorders (e.g., breast cancer, lung cancer), viral infections (e.g., herpes, papilloma, or HIV), and other conditions, such as osteoarthritis and atherosclerosis. The Bcl-xL inhibitor or ADCs described herein may be used to treat or ameliorate any of these diseases. Such treatments generally involve administering to a subject suffering from the disease an amount of a Bcl-xL inhibitor or ADC described herein sufficient to provide therapeutic benefit. For ADCs, identity of the antibody of the ADC administered will depend upon the disease being treated—to the antibody should bind a cell-surface antigen expressed in the cell type where inhibition of Bcl-xL activity would be beneficial. The therapeutic benefit achieved will also depend upon the specific disease being treated. In certain instances, the Bcl-xL inhibitor or ADC may treat or ameliorate the disease itself, or symptoms of the disease, when administered as monotherapy. In other instances, the Bcl-xL inhibitor or ADC may be part of an overall treatment regimen including other agents that, together with the inhibitor or ADC, treat or ameliorate the disease being treated, or symptoms of the disease. Agents useful to treat or ameliorate specific diseases that may be administered adjunctive to, or with, the Bcl-xL inhibitors and/or ADCs described herein will be apparent to those of skill in the art.

Although absolute cure is always desirable in any therapeutic regimen, achieving a cure is not required to provide therapeutic benefit. Therapeutic benefit may include halting or slowing the progression of the disease, regressing the disease without curing, and/or ameliorating or slowing the progression of symptoms of the disease. Prolonged survival as compared to statistical averages and/or improved quality of life may also be considered therapeutic benefit.

One particular class of diseases that involve dysregulated apoptosis and that are significant health burden world-wide are cancers. In a specific embodiment, the Bcl-xL inhibitors and/or ADCs described herein may be used to treat cancers. The cancer may be, for example, solid tumors or hematological tumors. Cancers that may be treated with the ADCs described herein include, but are not limited to bladder cancer, brain cancer, breast cancer, bone marrow cancer, cervical cancer, chronic lymphocytic leukemia, colorectal cancer, esophageal cancer, hepatocellular cancer, lymphoblastic leukemia, follicular lymphoma, lymphoid malignancies of T-cell or B-cell origin, melanoma, myelogenous leukemia, myeloma, oral cancer, ovarian cancer, non-small cell lung cancer, chronic lymphocytic leukemia, myeloma, prostate cancer, small cell lung cancer or spleen cancer. ADCs may be especially beneficial in the treatment of cancers because the antibody can be used to target the Bcl-xL inhibitory synthon specifically to tumor cells, thereby potentially avoiding or ameliorating undesirable side-effects and/or toxicities that may be associated with systemic administration of unconjugated inhibitors. In certain embodiments, the tumor cell is a SCLC tumor cell or NSCLC tumor cell.

In the context of tumorigenic cancers, therapeutic benefit, in addition to including the effects discussed above, may also specifically include halting or slowing progression of tumor growth, regressing tumor growth, eradicating one or more tumors and/or increasing patient survival as compared to statistical averages for the type and stage of the cancer being treated.

The Bcl-xL inhibitors and/or ADCs may be administered as monotherapy to provide therapeutic benefit, or may be administered adjunctive to, or with, other chemotherapeutic agents and/or radiation therapy. Chemotherapeutic agents to which the inhibitors and/or ADCs described herein may be utilized as adjunctive therapy may be targeted (for example, other Bcl-xL inhibitors or ADCs, protein kinase inhibitors, etc.) or non-targeted (for example, non-specific cytotoxic agents such as radionucleotides, alkylating agents and intercalating agents). Non-targeted chemotherapeutic agents with which the inhibitors and/or ADCs described herein may be adjunctively administered include, but are not limited to, methotrexate, taxol, L-asparaginase, mercaptopurine, thioguanine, hydroxyurea, cytarabine, cyclophosphamide, ifosfamide, nitrosoureas, cisplatin, carboplatin, mitomycin, dacarbazine, procarbazine, topotecan, nitrogen mustards, Cytoxan, etoposide, 5-fluorouracil, BCNU, irinotecan, camptothecins, bleomycin, doxorubicin, idarubicin, daunorubicin, dactinomycin, plicamycin, mitoxantrone, asparaginase, vinblastine, vincristine, vinorelbine, paclitaxel, calicheamicin, and docetaxel.

Elevated Bcl-xL expression has been shown to correlate with resistance to chemotherapy and radiation therapy. Data herein demonstrate that Bcl-xL inhibitors and/or ADCs that may not be effective as monotherapy to treat cancer may be administered adjunctive to, or with, other chemotherapeutic agents or radiation therapy to provide therapeutic benefit. While not intending to be bound by any therapy of operation, it is believed that administration of the Bcl-xL inhibitors and/or ADCs described herein to tumors that have become resistant to standard of care chemotherapeutic agents and/or radiation therapy sensitizes the tumors such that they again respond to the chemo and/or radiation therapy. Accordingly, in the context of treating cancers, “therapeutic benefit” includes administering the inhibitors and/or ADCs described herein adjunctive to, or with, chemotherapeutic agents and/or radiation therapy, either in patients who have not yet begin such therapy or who have but have not yet exhibited signs of resistance, or in patients who have begun to exhibit signs of resistance, as a means of sensitizing the tumors to the chemo and/or radiation therapy.

### 4.12. Dosages and Administration Regimens

The amount of Bcl-xL inhibitor and/or ADC administered will depend upon a variety of factors, including but not limited to, the particular disease being treated, the mode of administration, the desired therapeutic benefit, the stage or severity of the disease, the age, weight and other characteristics of the patient, etc. Determination of effective dosages is within the capabilities of those skilled in the art.

Effective dosages may be estimated initially from cellular assays. For example, an initial dose for use in humans may be formulated to achieve a circulating blood or serum concentration of Bcl-xL inhibitor or ADC that is expected to achieve a cellular concentration of Bcl-xL inhibitor that is at or above an IC₅₀ or ED₅₀ of the particular inhibitory molecule measured in a cellular assay.

Initial dosages for use in humans may also be estimated from in vivo animal models. Suitable animal models for a wide variety of diseases are known in the art.

When administered adjunctive to, or with, other agents, such as other chemotherapeutic agents, the Bcl-xL inhibitors or ADCs may be administered on the same schedule with the other agents, or on a different schedule. When administered on the same schedule, the inhibitor or ADC may be administered before, after, or concurrently with the other agent. In some embodiments where the inhibitor or ADC is administered adjunctive to, or with, standard chemo- and/or radiation therapy, the inhibitor or ADC may be initiated prior to commencement of the standard therapy, for example a day, several days, a week, several weeks, a month, or even several months before commencement of standard chemo- and/or radiation therapy.

When administered adjunctive to, or with, other agents, such as for example standard chemotherapeutic agents, the other agent will typically be administered according to its standard dosing schedule with respect to route, dosage and frequency. However, in some instances less than the standard amount may be necessary for efficacy when administered adjunctive to Bcl-xL inhibitor or ADC therapy.

### 5. EXAMPLES

### Example 1. Synthesis of Exemplary Bcl-xL Inhibitors

This example provides synthetic methods for exemplary Bcl-xL inhibitory compounds W2.01-W2.62. Bcl-xL inhibitors (W2.01-W2.91) and synthons (Examples 2.1-2.176) were named using ACD/Name 2012 release (Build 56084, 5 Apr. 2012, Advanced Chemistry Development Inc., Toronto, Ontario) or ACD/Name 2014 release (Build 66687, 25 Oct. 2013, Advanced Chemistry Development Inc., Toronto, Ontario). Bcl-xL inhibitor and synthon intermediates were named with ACD/Name 2012 release (Build 56084, 5 Apr. 2012, Advanced Chemistry Development Inc., Toronto, Ontario), ACD/Name 2014 release (Build 66687, 25 Oct. 2013, Advanced Chemistry Development Inc., Toronto, Ontario), ChemDraw® Ver. 9.0.7 (CambridgeSoft, Cambridge, MA), ChemDraw® Ultra Ver. 12.0 (CambridgeSoft, Cambridge, MA), or ChemDraw® Professional Ver. 15.0.0.106.

### 1.1. Synthesis of 6-[8-(1,3-benzothiazol-2-ylcarbamoyl)-3,4-dihydroisoquinolin-2(1H)-yl]-3-[1-({3-[2-({2-[2-(carboxymethoxy)ethoxy]ethyl}amino)ethoxy]-5,7-dimethyltricyclo[3.3.1.1^{3,7}]dec-1-yl}methyl)-5-methyl-1H-pyrazol-4-yl]pyridine-2-carboxylic Acid (Compound W2.01)

### 1.1.1. 3-bromo-5,7-dimethyladamantanecarboxylic Acid

Into a 50 mL round-bottomed flask at 0° C., was added bromine (16 mL). Iron powder (7 g) was added, and the reaction was stirred at 0° C. for 30 minutes. 3,5-Dimethyladamantane-1-carboxylic acid (12 g) was added. The mixture was warmed up to room temperature and stirred for 3 days. A mixture of ice and concentrated HCl was poured into the reaction mixture. The resulting suspension was treated twice with Na₂SO₃ (50 g in 200 mL water) and extracted three times with dichloromethane. The combined organics were washed with 1N aqueous HCl, dried over sodium sulfate, filtered, and concentrated to give the title compound.

### 1.1.2. 3-bromo-5,7-dimethyladamantanemethanol

To a solution of Example 1.1.1 (15.4 g) in tetrahydrofuran (200 mL) was added BH₃ (1M in tetrahydrofuran, 150 mL), and the mixture was stirred at room temperature overnight. The reaction mixture was then carefully quenched by adding methanol dropwise. The mixture was then concentrated under vacuum, and the residue was balanced between ethyl acetate (500 mL) and 2N aqueous HCl (100 mL). The aqueous layer was further extracted twice with ethyl acetate, and the combined organic extracts were washed with water and brine, dried over sodium sulfate, and filtered. Evaporation of the solvent gave the title compound.

### 1.1.3. 1-((3-bromo-5,7-dimethyltricyclo[3.3.1.1^{3,7}]dec-1-yl)methyl)-1H-pyrazole

To a solution of Example 1.1.2 (8.0 g) in toluene (60 mL) was added 1H-pyrazole (1.55 g) and cyanomethylenetributylphosphorane (2.0 g), and the mixture was stirred at 90° C. overnight. The reaction mixture was concentrated, and the residue was purified by silica gel column chromatography (10:1 heptane:ethyl acetate) to give the title compound. MS (ESI) m/e 324.2 (M+H)⁺.

### 1.1.4. 2-{[3,5-dimethyl-7-(1H-pyrazol-1-ylmethyl)tricyclo[3.3.1.1^{3,7}]dec-1-yl]oxy}ethanol

To a solution of Example 1.1.3 (4.0 g) in ethane-1,2-diol (12 mL) was added triethylamine (3 mL). The mixture was stirred at 150° C. under microwave conditions (Biotage Initiator) for 45 minutes. The mixture was poured into water (100 mL) and extracted three times with ethyl acetate. The combined organic extracts were washed with water and brine, dried over sodium sulfate, and filtered. Evaporation of the solvent gave a residue that was purified by silica gel chromatography, eluting with 20% ethyl acetate in heptane, followed by 5% methanol in dichloromethane, to give the title compound. MS (ESI) m/e 305.2 (M+H)⁺.

### 1.1.5. 2-({3,5-dimethyl-7-[(5-methyl-1H-pyrazol-1-yl)methyl]tricyclo[3.3.1.1^{3,7}]dec-1-yl}oxy)ethanol

To a cooled (−78° C.) solution of Example 1.1.4 (6.05 g) in tetrahydrofuran (100 mL) was added n-BuLi (40 mL, 2.5M in hexane), and the mixture was stirred at −78° C. for 1.5 hours. Iodomethane (10 mL) was added through a syringe, and the mixture was stirred at −78° C. for 3 hours. The reaction mixture was then quenched with aqueous NH₄Cl and extracted twice with ethyl acetate, and the combined organic extracts were washed with water and brine. After drying over sodium sulfate, the solution was filtered and concentrated, and the residue was purified by silica gel column chromatography, eluting with 5% methanol in dichloromethane, to give the title compound. MS (ESI) m/e 319.5 (M+H)⁺.

### 1.1.6. 1-({3,5-dimethyl-7-[2-(hydroxy)ethoxy]tricyclo[3.3.1.1^{3,7}]dec-1-yl}methyl)-4-iodo-5-methyl-1H-pyrazole

To a solution of Example 1.1.5 (3.5 g) in N,N-dimethylformamide (30 mL) was added N-iodosuccinimide (3.2 g), and the mixture was stirred at room temperature for 1.5 hours. The reaction mixture was diluted with ethyl acetate (600 mL) and washed with aqueous NaHSO₃, water and brine. The organic layer was dried over sodium sulfate, filtered and concentrated under reduced pressure. The residue was purified by silica gel chromatography, eluting with 20% ethyl acetate in dichloromethane, to give the title compound. MS (ESI) m/e 445.3 (M+H)⁺.

### 1.1.7. 1-((3-(2-((tert-butyldimethylsilyl)oxy)ethoxy)-5,7-dimethyladamantan-1-yl)methyl)-4-iodo-5-methyl-1H-pyrazole

Tert-butyldimethylsilyl trifluoromethanesulfonate (5.34 mL) was added to a solution of Example 1.1.6 (8.6 g) and 2,6-lutidine (3.16 mL) in dichloromethane (125 mL) at −40° C., and the reaction was allowed to warm to room temperature overnight. The mixture was concentrated, and the residue was purified by silica gel chromatography, eluting with 5-20% ethyl acetate in heptanes, to give the title compound. MS (ESI) m/e 523.4 (M+H)⁺.

### 1.1.8. 1-((3-(2-((tert-butyldimethylsilyl)oxy)ethoxy)-5,7-dimethyladamantan-1-yl)methyl)-5-methyl-4-(4,4,5,5-tetramethyl-1,3,2-dioxaborolan-2-yl)-1H-pyrazole

n-Butyllithium (8.42 mL, 2.5M in hexanes) was added to Example 1.1.7 (9.8 g) in 120 mL tetrahydrofuran at −78° C., and the reaction was stirred for 1 minute. Trimethyl borate (3.92 mL) was added, and the reaction stirred for 5 minutes. Pinacol (6.22 g) was added, and the reaction was allowed to warm to room temperature and was stirred 2 hours. The reaction was quenched with pH 7 buffer, and the mixture was poured into ether. The layers were separated, and the organic layer was concentrated under reduced pressure. The residue was purified by silica gel chromatography, eluting with 1-25% ethyl acetate in heptanes, to give the title compound.

### 1.1.9. 6-fluoro-3-bromopicolinic Acid

A slurry of 6-amino-3-bromopicolinic acid (25 g) in 400 mL 1:1 dichloromethane/chloroform was added to nitrosonium tetrafluoroborate (18.2 g) in dichloromethane (100 mL) at 5° C. over 1 hour. The resulting mixture was stirred for another 30 minutes, then warmed to 35° C. and stirred overnight. The reaction was cooled to room temperature, and then adjusted to pH 4 with aqueous NaH₂PO₄ solution. The resulting solution was extracted three times with dichloromethane, and the combined extracts were washed with brine, dried over sodium sulfate, filtered and concentrated to provide the title compound.

### 1.1.10. Tert-butyl 3-bromo-6-fluoropicolinate

Para-toluenesulfonyl chloride (27.6 g) was added to a solution of Example 1.1.9 (14.5 g) and pyridine (26.7 mL) in dichloromethane (100 mL) and tert-butanol (80 mL) at 0° C. The reaction was stirred for 15 minutes, and then warmed to room temperature, and stirred overnight. The solution was concentrated and partitioned between ethyl acetate and aqueous Na₂CO₃ solution. The layers were separated, and the aqueous layer extracted with ethyl acetate. The organic layers were combined, rinsed with aqueous Na₂CO₃ solution and brine, dried over sodium sulfate, filtered, and concentrated to provide the title compound.

### 1.1.11. Methyl 2-(5-bromo-6-(tert-butoxycarbonyl)pyridin-2-yl)-1,2,3,4-tetrahydroisoquinoline-8-carboxylate

To a solution of methyl 1,2,3,4-tetrahydroisoquinoline-8-carboxylate hydrochloride (12.37 g) and Example 1.1.10 (15 g) in dimethyl sulfoxide (100 mL) was added N,N-diisopropylethylamine (12 mL), and the mixture was stirred at 50° C. for 24 hours. The mixture was then diluted with ethyl acetate (500 mL) and washed with water and brine. The organic layer was dried over sodium sulfate, filtered and concentrated under reduced pressure. The residue was purified by silica gel chromatography, eluting with 20% ethyl acetate in hexane, to give the title compound. MS (ESI) m/e 448.4 (M+H)⁺.

### 1.1.12. Methyl 2-(6-(tert-butoxycarbonyl)-5-(1-((3-(2-((tert-butyldimethylsilyl)oxy)ethoxy)-5,7-dimethyladamantan-1-yl)methyl)-5-methyl-1H-pyrazol-4-yl)pyridin-2-yl)-1,2,3,4-tetrahydroisoquinoline-8-carboxylate

A mixture of Example 1.1.11 (3.08 g), Example 1.1.8 (5 g), tris(dibenzylideneacetone)dipalladium(0) (126 mg), 1,3,5,7-tetramethyl-8-tetradecyl-2,4,6-trioxa-8-phosphaadamantane (170 mg), and K₃PO₄ (3.65 g) in 1,4-dioxane (25 mL) and water (25 mL) was heated to 90° C. for 2 hours. The mixture was cooled and poured into 1:1 diethyl ether:ethyl acetate. The layers were separated, and the organic was washed with saturated aqueous NaH₂PO₄ solution, water (2×), and brine. The organic layer was dried over sodium sulfate, filtered, and concentrated. The residue was purified by silica gel chromatography, eluting with 1-25% ethyl acetate in heptanes, to give the title compound. MS (ESI) m/e 799.6 (M+H)⁺.

### 1.1.13. 2-(6-(tert-butoxycarbonyl)-5-(1-((3-(2-((tert-butyldimethylsilyl)oxy)ethoxy)-5,7-dimethyladamantan-1-yl)methyl)-5-methyl-1H-pyrazol-4-yl)pyridin-2-yl)-1,2,3,4-tetrahydroisoquinoline-8-carboxylic acid

Example 1.1.12 (5 g) and lithium hydroxide monohydrate (0.276 g) were stirred together in a solvent mixture of tetrahydrofuran (50 mL), methanol (5 mL) and water (15 mL) at 70° C. for 2 days. The reaction was cooled, acidified with 1M aqueous HCl solution, and extracted twice with ethyl acetate. The combined organic layers were washed with brine, dried over sodium sulfate, filtered, and concentrated. The residue was dissolved in dichloromethane (100 mL), cooled at −40° C., and 2,6-lutidine (1.8 mL) and tert-butyldimethylsilyl trifluoromethanesulfonate (3.28 g) were added. The reaction was allowed to warm to room temperature and was stirred for 2 hours. The mixture was diluted with ether, and the layers were separated. The organic layer was concentrated. The residue was dissolved in tetrahydrofuran and treated with saturated aqueous K₂CO₃ solution for 1 hour. This mixture was acidified with concentrated HCl and extracted twice with ethyl acetate. The combined organic layers were dried over sodium sulfate, filtered, and concentrated under reduced pressure. The residue was purified by silica gel chromatography, eluting with 10-100% ethyl acetate in heptanes then 5% methanol in ethyl acetate, to give the title compound. MS (ESI) m/e 785.6 (M+H)⁺.

### 1.1.14. tert-butyl 6-(8-(benzo[d]thiazol-2-ylcarbamoyl)-3,4-dihydroisoquinolin-2(1H)-yl)-3-(1-((3-(2-hydroxyethoxy)-5,7-dimethyladamantan-1-yl)methyl)-5-methyl-1H-pyrazol-4-yl)picolinate

Example 1.1.13 (970 mg), N,N-diisopropylethylamine (208 mg), and 2-(3H-[1,2,3]triazolo[4,5-b]pyridin-3-yl)-1,1,3,3-tetramethylisouronium hexafluorophosphate (HATU) (970 mg) were stirred in 7 mL N,N-dimethylformamide at 0° C. for 10 minutes. Benzo[d]thiazol-2-amine (278 mg) was added, and the mixture was stirred for 24 hours at 50° C. The mixture was cooled and diluted with ethyl acetate. The organic layer was washed with water and brine, dried over sodium sulfate, filtered, and concentrated. The residue was dissolved in tetrahydrofuran (50 mL), and tetrabutyl ammonium fluoride (10 mL, 1M in tetrahydrofuran) was added. The reaction was stirred for 1 hour, poured into ethyl acetate and washed with pH 7 buffer and brine. The organic layer was dried over sodium sulfate, filtered, and concentrated under reduced pressure. The residue was purified by silica gel chromatography, eluting with 10-100% ethyl acetate in heptanes, to give the title compound. MS (ESI) m/e 803.7 (M+H)⁺.

### 1.1.15. tert-butyl 6-(8-(benzo[d]thiazol-2-ylcarbamoyl)-3,4-dihydroisoquinolin-2(1H)-yl)-3-(1-((3,5-dimethyl-7-(2-oxoethoxy)adamantan-1-yl)methyl)-5-methyl-1H-pyrazol-4-yl)picolinate

To an ambient solution of Example 1.1.14 (100 mg) in dichloromethane (1.3 mL) was added Dess-Martin periodinane (58.1 mg) in a single portion. The reaction was stirred for 0.5 hours, and additional Dess-Martin periodinane (8 mg) was added. The reaction was stirred for 1 hour and quenched by the addition of ˜10% aqueous NaOH solution and dichloromethane. The layers were separated, and the organic layer was washed with ˜10% aqueous NaOH solution. The organic layer was dried with anhydrous sodium sulfate, filtered and concentrated under reduced pressure to a solid, which was used in the subsequent reaction without further purification. MS (ESI) m/e 801.3 (M+H)⁺.

### 1.1.16. 2-(2-(2-((2-((3-((4-(6-(8-(benzo[d]thiazol-2-ylcarbamoyl)-3,4-dihydroisoquinolin-2(1H)-yl)-2-(tert-butoxycarbonyl)pyridin-3-yl)-5-methyl-1H-pyrazol-1-yl)methyl)-5,7-dimethyladamantan-1-yl)oxy)ethyl)amino)ethoxy)ethoxy)acetic acid

To an ambient solution of 2-(2-(2-aminoethoxy)ethoxy)acetic acid (22 mg) and Example 1.1.15 (100 mg) in methanol (1.3 mL) was added MP-CNBH₃ (65 mg, 2.49 mmol/g loading). The reaction was gently shaken overnight and filtered through a 0.4 micron filter. The crude material was purified by reverse phase HPLC using a Gilson system, eluting with 20-80% acetonitrile in water containing 0.1% v/v trifluoroacetic acid. The desired fractions were combined and freeze-dried to provide the title compound. MS (ESI) m/e 948.3 (M+H)⁺.

### 1.1.17. 6-(8-(benzo[d]thiazol-2-ylcarbamoyl)-3,4-dihydroisoquinolin-2(1H)-yl)-3-(1-((3-(2-((2-(2-(carboxymethoxy)ethoxy)ethyl)amino)ethoxy)-5,7-dimethyladamantan-1-yl)methyl)-5-methyl-1H-pyrazol-4-yl)picolinic Acid

To an ambient solution of Example 1.1.16 (15 mg) in dichloromethane (1 mL) was added trifluoroacetic acid (1 mL). The reaction was stirred for 16 hours and then concentrated under reduced pressure. The residue was purified by reverse phase HPLC using a Gilson system, eluting with 20-80% acetonitrile in water containing 0.1% v/v trifluoroacetic acid. The desired fractions were combined and freeze-dried to provide the title compound. ¹H NMR (400 MHz, dimethyl sulfoxide-d₆) δ ppm 12.70 (bs, 2H), 8.29 (s, 1H), 8.03 (d, 1H), 7.79 (d, 1H), 7.62 (d, 1H), 7.53-7.42 (m, 3H), 7.40-7.32 (m, 2H), 7.29 (s, 1H), 6.96 (d, 1H), 4.96 (bs, 2H), 4.03 (s, 2H), 3.90 (t, 2H), 3.84 (s, 2H), 3.68 (t, 2H), 3.63-3.54 (m, 6H), 3.17-3.04 (m, 4H), 3.00 (t, 2H), 2.10 (s, 3H), 1.45-1.40 (m, 2H), 1.36-1.20 (m, 4H), 1.21-0.96 (m, 7H), 0.91-0.81 (m, 6H). MS (ESI) m/e 892.3 (M+H)⁺.

### 1.2. Synthesis of 6-[8-(1,3-benzothiazol-2-ylcarbamoyl)-3,4-dihydroisoquinolin-2(1H)-yl]-3-{1-[(3,5-dimethyl-7-{2-[(2-sulfoethyl)amino]ethoxy}tricyclo[3.3.1.1^{3,7}]dec-1-yl)methyl]-5-methyl-1H-pyrazol-4-yl}pyridine-2-carboxylic Acid (Compound W2.02)

### 1.2.1. Methyl 2-(6-(tert-butoxycarbonyl)-5-(4,4,5,5-tetramethyl-1,3,2-dioxaborolan-2-yl)pyridin-2-yl)-1,2,3,4-tetrahydroisoquinoline-8-carboxylate

To a solution of Example 1.1.11 (2.25 g) and [1,1′-bis(diphenylphosphino)ferrocene]dichloropalladium(II)(205 mg) in acetonitrile (30 mL) was added triethylamine (3 mL) and pinacolborane (2 mL), and the mixture was stirred at reflux for 3 hours. The mixture was diluted with ethyl acetate (200 mL) and washed with water and brine. The organic layer was dried over sodium sulfate, filtered and concentrated under reduced pressure. Purification of the residue by silica gel chromatography, eluting with 20% ethyl acetate in hexane, provided the title compound.

### 1.2.2. Methyl 2-(6-(tert-butoxycarbonyl)-5-(1-((3-(2-hydroxyethoxy)-5,7-dimethyladamantan-1-yl)methyl)-5-methyl-1H-pyrazol-4-yl)pyridin-2-yl)-1,2,3,4-tetrahydroisoquinoline-8-carboxylate

To a solution of Example 1.2.1 (2.25 g) in tetrahydrofuran (30 mL) and water (10 mL) was added Example 1.1.6 (2.0 g), 1,3,5,7-tetramethyl-6-phenyl-2,4,8-trioxa-6-phosphaadamantane (329 mg), tris(dibenzylideneacetone)dipalladium(0) (206 mg) and potassium phosphate tribasic (4.78 g). The mixture was refluxed overnight, cooled and diluted with ethyl acetate (500 mL). The resulting mixture was washed with water and brine, and the organic layer was dried over sodium sulfate, filtered and concentrated. The residue was purified by flash chromatography, eluting with 20% ethyl acetate in heptanes followed by 5% methanol in dichloromethane, to provide the title compound.

### 1.2.3. Methyl 2-(6-(tert-butoxycarbonyl)-5-(1-((3,5-dimethyl-7-(2-((methylsulfonyl)oxy)ethoxy)adamantan-1-yl)methyl)-5-methyl-1H-pyrazol-4-yl)pyridin-2-yl)-1,2,3,4-tetrahydroisoquinoline-8-carboxylate

To a cold solution of Example 1.2.2 (3.32 g) in dichloromethane (100 mL) in an ice-bath was sequentially added triethylamine (3 mL) and methanesulfonyl chloride (1.1 g). The reaction mixture was stirred at room temperature for 1.5 hours and diluted with ethyl acetate, and washed with water and brine. The organic layer was dried over sodium sulfate, filtered, and concentrated to provide the title compound.

### 1.2.4. Methyl 2-(5-(1-((3-(2-azidoethoxy)-5,7-dimethyladamantan-1-yl)methyl)-5-methyl-1H-pyrazol-4-yl)-6-(tert-butoxycarbonyl)pyridin-2-yl)-1,2,3,4-tetrahydroisoquinoline-8-carboxylate

To a solution of Example 1.2.3 (16.5 g) in N,N-dimethylformamide (120 mL) was added sodium azide (4.22 g). The mixture was heated at 80° C. for 3 hours, cooled, diluted with ethyl acetate and washed with water and brine. The organic layer was dried over sodium sulfate, filtered, and concentrated. The residue was purified by flash chromatography, eluting with 20% ethyl acetate in heptanes, to provide the title compound.

### 1.2.5. 2-(5-(1-((3-(2-azidoethoxy)-5,7-dimethyladamantan-1-yl)methyl)-5-methyl-1H-pyrazol-4-yl)-6-(tert-butoxycarbonyl)pyridin-2-yl)-1,2,3,4-tetrahydroisoquinoline-8-carboxylic acid

To a solution of Example 1.2.4 (10 g) in a mixture of tetrahydrofuran (60 mL), methanol (30 mL) and water (30 mL) was added lithium hydroxide monohydrate (1.2 g). The mixture was stirred at room temperature overnight and neutralized with 2% aqueous HCl. The resulting mixture was concentrated, and the residue was dissolved in ethyl acetate (800 mL), and washed with brine. The organic layer was dried over sodium sulfate, filtered, and concentrated to provide the title compound.

### 1.2.6. tert-butyl 3-(1-((3-(2-azidoethoxy)-5,7-dimethyladamantan-1-yl)methyl)-5-methyl-1H-pyrazol-4-yl)-6-(8-(benzo[d]thiazol-2-ylcarbamoyl)-3,4-dihydroisoquinolin-2(1H)-yl)picolinate

A mixture of Example 1.2.5 (10 g), benzo[d]thiazol-2-amine (3.24 g), fluoro-N,N,N′,N′-tetramethylformamidinium hexafluorophosphate (5.69 g) and N,N-diisopropylethylamine (5.57 g) in N,N-dimethylformamide (20 mL) was heated at 60° C. for 3 hours, cooled and diluted with ethyl acetate. The resulting mixture was washed with water and brine. The organic layer was dried over sodium sulfate, filtered, and concentrated. The residue was purified by flash chromatography, eluting with 20% ethyl acetate in dichloromethane to give the title compound.

### 1.2.7. tert-butyl 3-(1-(((3-(2-aminoethoxy)-5,7-dimethyladamantan-1-yl)methyl)-5-methyl-1H-pyrazol-4-yl)-6-(8-(benzo[d]thiazol-2-ylcarbamoyl)-3,4-dihydroisoquinolin-2(1H)-yl)picolinate

To a solution of Example 1.2.6 (2.0 g) in tetrahydrofuran (30 mL) was added Pd/C (10%, 200 mg). The mixture was stirred under a hydrogen atmosphere overnight. The insoluble material was filtered off and the filtrate was concentrated to provide the title compound.

### 1.2.8. tert-butyl 6-[8-(1,3-benzothiazol-2-ylcarbamoyl)-3,4-dihydroisoquinolin-2(1H)-yl]-3-[1-({3,5-dimethyl-7-[(2,2,7,7-tetramethyl-10,10-dioxido-3,3-diphenyl-4,9-dioxa-10λ⁶-thia-13-aza-3-silapentadecan-15-yl)oxy]tricyclo[3.3.1.1^{3,7}]dec-1-yl}methyl)-5-methyl-1H-pyrazol-4-yl]pyridine-2-carboxylate

To a solution of Example 1.2.7 (500 mg) in N,N-dimethylformamide (8 mL) was added 4-((tert-butyldiphenylsilyl)oxy)-2,2-dimethylbutyl ethanesulfonate (334 mg). The reaction was stirred at room temperature overnight and methylamine (0.3 mL) was added to quench the reaction. The resulting mixture was stirred for 20 minutes and purified by reverse-phase chromatography using an Analogix system (C18 column), eluting with 50-100% acetonitrile in water containing 0.1% v/v trifluoroacetic acid, to provide the title compound.

### 1.2.9. 6-[8-(1,3-benzothiazol-2-ylcarbamoyl)-3,4-dihydroisoquinolin-2(1H)-yl]-3-{1-[(3,5-dimethyl-7-{2-[(2-sulfoethyl)amino]ethoxy}tricyclo[3.3.1.1^{3,7}]dec-1-yl)methyl]-5-methyl-1H-pyrazol-4-yl}pyridine-2-carboxylic Acid

Example 1.2.8 (200 mg) in dichloromethane (5 mL) was treated with trifluoroacetic acid (2.5 mL) overnight. The reaction mixture was concentrated and purified by reverse phase chromatography (C18 column), eluting with 20-60% acetonitrile in water containing 0.1% v/v trifluoroacetic acid, to provide the title compound. ¹H NMR (500 MHz, dimethyl sulfoxide-d₆) δ ppm 12.86 (s, 1H), 8.32 (s, 2H), 8.02 (d, 1H), 7.78 (d, 1H), 7.60 (d, 1H), 7.51 (d, 1H), 7.40-7.49 (m, 2H), 7.31-7.39 (m, 2H), 7.27 (s, 1H), 6.95 (d, 1H), 4.94 (s, 2H), 3.87 (t, 2H), 3.81 (s, 2H), 3.15-3.25 (m, 2H), 3.03-3.13 (m, 2H), 3.00 (t, 2H), 2.79 (t, 2H), 2.09 (s, 3H), 1.39 (s, 2H), 1.22-1.34 (m, 4H), 0.94-1.18 (m, 6H), 0.85 (s, 6H). MS (ESI) m/e 854.1 (M+H)⁺.

### 1.3. Synthesis of 2-{[(2-{[2-({3-[(4-{6-[8-(1,3-benzothiazol-2-ylcarbamoyl)-3,4-dihydroisoquinolin-2(1H)-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl]amino}ethyl)sulfonyl]amino}-2-deoxy-D-glucopyranose (Compound W2.03)

### 1.3.1. 3-(1-((3-(2-aminoethoxy)-5,7-dimethyladamantan-1-yl)methyl)-5-methyl-1H-pyrazol-4-yl)-6-(8-(benzo[d]thiazol-2-ylcarbamoyl)-3,4-dihydroisoquinolin-2(1H)-yl)picolinic Acid

Example 1.2.7 (200 mg) in dichloromethane (2.5 mL) was treated with trifluoroacetic acid (2.5 mL) overnight. The reaction mixture was concentrated, and the residue was purified by reverse phase chromatography (C18 column), eluting with 20-60% acetonitrile in water containing 0.1% v/v trifluoroacetic acid, to provide the title compound. MS (ESI) m/e 746.2 (M+H)⁺.

### 1.3.2. (3R,4R,5S,6R)-6-(acetoxymethyl)-3-(vinylsulfonamido)tetrahydro-2H-pyran-2,4,5-triyl Triacetate

To a suspension of (3R,4R,5S,6R)-6-(acetoxymethyl)-3-aminotetrahydro-2H-pyran-2,4,5-triyl triacetate (7.7 g) in dichloromethane (100 mL) at 0° C. was added 2-chloroethanesulfonyl chloride (4.34 g). The mixture was stirred at 0° C. for 15 minutes, and triethylamine (12.1 mL) was added. The mixture was stirred at 0° C. for 1 hour, warmed to room temperature and stirred for 2 days. The mixture was diluted with dichloromethane and washed with water and brine. The organic layer was dried over sodium sulfate, filtered, and concentrated to provide the title compound. N-((3R,4R,5S,6R)-2,4,5-trihydroxy-6-(hydroxymethyl)tetrahydro-2H-pyran-3-yl)ethenesulfonamide

To a solution of Example 1.3.2 (6.74 g) in methanol (150 mL) was added triethylamine (10 mL). The mixture was stirred for 4 days and concentrated. The residue was dissolved in methanol and treated with Dowex HCR-5 until the solution was neutral. The mixture was filtered, and the filtrate was concentrated. The residue was purified by chromatography using a column of Sephadex LH-20 (100 g), eluting with methanol to provide the title compound.

### 1.3.3. 2-{[(2-{[2-({3-[(4-{6-[8-(1,3-benzothiazol-2-ylcarbamoyl)-3,4-dihydroisoquinolin-2(1H)-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl]amino}ethyl)sulfonyl]amino}-2-deoxy-D-glucopyranose

A mixture of Example 1.3.1 (23.5 mg), Example 1.3.3 (42.4 mg), and N,N-diisopropylethylamine (55 μl) in N,N-dimethylformamide (1 mL) and water (0.3 mL) was stirred for 5 days. The mixture was purified by reverse phase chromatography (C18 column), eluting with 20-60% acetonitrile in water containing 0.1% v/v trifluoroacetic acid, to provide the title compound. ¹H NMR (400 MHz, dimethyl sulfoxide-d₆) δ ppm 12.85 (s, 1H), 8.42 (s, 1H), 8.42 (s, 1H), 8.03 (d, 1H), 7.79 (d, 1H), 7.55-7.66 (m, 1H), 7.46-7.54 (m, 2H), 7.42-7.47 (m, 1H), 7.33-7.40 (m, 2H), 7.29 (s, 1H), 6.96 (d, 1H), 4.96 (s, 2H), 3.89 (t, 2H), 3.83 (s, 2H), 2.97-3.14 (m, 6H), 2.10 (s, 3H), 1.44 (s, 2H), 1.22-1.39 (m, 4H), 0.97-1.20 (m, 6H), 0.87 (s, 6H). MS (ESI) m/e 1015.3 (M+H)⁺.

This paragraph was intentionally left blank.

### 1.4. Synthesis of (1xi)-1,5-anhydro-1-[4-({[2-({3-[(4-{6-[8-(1,3-benzothiazol-2-ylcarbamoyl)-3,4-dihydroisoquinolin-2(1H)-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl]amino}methyl)benzyl]-D-glucitol (Compound W2.05)

### 1.4.1. [4-((3S,4R,5R,6R)-3,4,5-Tris-methoxymethoxy-6-methoxymethoxymethyl-tetrahydro-pyran-2-ylmethyl)-phenyl]-methanol

The title compound was prepared according to J. R. Walker et al., Bioorg. Med. Chem. 2006, 14, 3038-3048. MS (ESI) m/e 478 (M+NH₄)⁺.

### 1.4.2. 4-((3S,4R,5R,6R)-3,4,5-Tris-methoxymethoxy-6-methoxymethoxymethyl-tetrahydro-pyran-2-ylmethyl)-benzaldehyde

Example 1.5.1 (1.000 g) was dissolved in dichloromethane (25 mL), and Dess-Martin periodinane (1.013 g) was added. The solution was stirred 16 hours at room temperature. The solution was diluted with diethyl ether (25 mL) and 2 M aqueous sodium carbonate solution (25 mL) was added. The mixture was extracted with diethyl ether three times. The organic extracts were combined, washed with brine, and dried over anhydrous sodium sulfate. After filtration, the solution was concentrated under reduced pressure and purified by silica gel chromatography, eluting with 50-70% ethyl acetate in heptanes. The solvent was evaporated under reduced pressure to provide the title compound. MS (ESI) m/e 476 (M+NH₄)⁺.

### 1.4.3. Acetic Acid (2R,3R,4R,5S)-3,4,5-triacetoxy-6-(4-formyl-benzyl)-tetrahydro-pyran-2-ylmethyl Ester

Example 1.5.2 (660 mg) was dissolved in methanol (145 mL). 6 M Hydrochloric acid (8 mL) was added, and the solution was stirred at room temperature for two days. The solvents were removed under reduced pressure, azeotroping with ethyl acetate three times. The material was dried under vacuum for four days. The material was dissolved in N,N-dimethylformamide (50 mL). Acetic anhydride (12 mL), pyridine (6 mL), and N,N-dimethylpyridin-4-amine (10 mg) were added sequentially, and the solution was stirred at room temperature for 16 hours. The solution was diluted with water (150 mL) and extracted with ethyl acetate (50 mL) three times. The organics were combined, washed with water, washed with brine, and dried over anhydrous sodium sulfate. After filtration, the solution was concentrated under reduced pressure and purified by chromatography on silica gel, eluting with 40-50% ethyl acetate in heptanes. The solvent was evaporated under reduced pressure to provide the title compound.

### 1.4.4. (2R,3R,4R,5S)-2-(acetoxymethyl)-6-(4-(((2-((3-((4-(6-(8-(benzo[d]thiazol-2-ylcarbamoyl)-3,4-dihydroisoquinolin-2(1H)-yl)-2-(tert-butoxycarbonyl)pyridin-3-yl)-5-methyl-1H-pyrazol-1-yl)methyl)-5,7-dimethyladamantan-1-yl)oxy)ethyl)amino)methyl)benzyl)tetrahydro-2H-pyran-3,4,5-triyl Triacetate

Example 1.2.7 (40 mg) and Example 1.5.3 (22.5 mg) were stirred in dichloromethane (1 mL) at room temperature for 10 minutes. Sodium triacetoxyborohydride (14 mg) was added, and the solution was stirred at room temperature for 16 hours. The material was purified by chromatography on silica gel, eluting with 10% methanol in dichloromethane. The solvent was evaporated under reduced pressure to provide the title compound. MS (ESI) m/e 1236 (M+H)⁺.

### 1.4.5. (1xi)-1,5-anhydro-1-[4-({[2-({3-[(4-{6-[8-(1,3-benzothiazol-2-ylcarbamoyl)-3,4-dihydroisoquinolin-2(1H)-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl]amino}methyl)benzyl]-D-glucitol

Example 1.5.4 (68 mg) was dissolved in methanol (0.5 mL). Aqueous lithium hydroxide solution (2M, 1 mL) was added, and the solution was stirred at room temperature for 4.5 hours. Acetic acid (0.1 mL) was added, and the solvents were removed under vacuum. The material was then dissolved in trifluoroacetic acid (2 mL) and stirred at room temperature for 16 hours. The solution was concentrated under vacuum. The residue was purified by reverse phase HPLC using a Gilson PLC 2020 with a 150×30 mm C18 column, eluting with 20-70% acetonitrile in water containing 0.1% v/v trifluoroacetic acid. The desired fractions were combined and freeze-dried to provide the title compound. ¹H NMR (400 MHz, dimethyl sulfoxide-d₆) δ ppm 12.86 (bs, 1H), 8.68 (bs, 2H), 8.04 (d, 1H), 7.80 (d, 1H), 7.62 (d, 1H), 7.51-7.43 (m, 3H), 7.39-7.24 (m, 6H), 6.96 (d, 1H), 5.23 (t, 1H), 4.96 (s, 2H), 4.56 (d, 1H), 4.42 (dd, 1H), 4.11 (m, 2H), 3.89 (t, 2H), 3.83 (s, 2H), 3.61-3.56 (m, 3H), 3.39 (dd, 1H), 3.22 (t, 1H), 3.15 (t, 1H), 3.09 (d, 1H), 3.01 (m, 6H), 2.89 (t, 1H), 2.60 (m, 1H), 2.10 (s, 3H), 1.43 (s, 2H), 1.30 (q, 4H), 1.14 (m, 4H), 1.03 (q, 2H), 0.86 (s, 6H). MS (ESI) m/e 1012 (M+H)⁺.

### 1.5. Synthesis of 6-[8-(1,3-benzothiazol-2-ylcarbamoyl)-3,4-dihydroisoquinolin-2(1H)-yl]-3-{1-[(3,5-dimethyl-7-{2-[(3-sulfopropyl)amino]ethoxy}tricyclo[3.3.1.1^{3,7}]dec-1-yl)methyl]-5-methyl-1H-pyrazol-4-yl}pyridine-2-carboxylic Acid (Compound W2.06)

### 1.5.1. 3-((2-((3-((4-(6-(8-(benzo[d]thiazol-2-ylcarbamoyl)-3,4-dihydroisoquinolin-2(1H)-yl)-2-(tert-butoxycarbonyl)pyridin-3-yl)-5-methyl-1H-pyrazol-1-yl)methyl)-5,7-dimethyladamantan-1-yl)oxy)ethyl)amino)propane-1-sulfonic Acid

A mixture of Example 1.2.7 (100 mg), 1,2-oxathiolane 2,2-dioxide (13 mg) and N,N-diisopropylethylamine (19.07 μL) in N,N-dimethylformamide (2 mL) was heated to 50° C. overnight. The reaction was cooled and purified by reverse phase HPLC (C18 column), eluting with 20-60% acetonitrile in water containing 0.1% v/v trifluoroacetic acid, to provide the title compound. MS (ESI) m/e 924.1 (M+H)⁺.

### 1.5.2. 6-[8-(1,3-benzothiazol-2-ylcarbamoyl)-3,4-dihydroisoquinolin-2(1H)-yl]-3-{1-[(3,5-dimethyl-7-{2-[(3-sulfopropyl)amino]ethoxy}tricyclo[3.3.1.1^{3,7}]dec-1-yl)methyl]-5-methyl-1H-pyrazol-4-yl}pyridine-2-carboxylic Acid

Example 1.6.1 (40 mg) in dichloromethane (2.5 mL) was treated with trifluoroacetic acid (2.5 mL) overnight. The reaction mixture was concentrated, and the residue was purified by reverse phase chromatography (C18 column), eluting with 20-60% acetonitrile in water containing 0.1% v/v trifluoroacetic acid, to provide the title compound. ¹H NMR (400 MHz, dimethyl sulfoxide-d₆) δ ppm 12.86 (s, 1H), 8.52 (s, 2H), 8.04 (d, 1H), 7.79 (d, 1H), 7.61 (d, 1H), 7.41-7.55 (m, 3H), 7.32-7.39 (m, 2H), 7.29 (s, 1H), 6.96 (d, 1H), 4.96 (s, 2H), 3.89 (t, 2H), 3.49-3.58 (m, 2H), 2.94-3.12 (m, 6H), 2.56-2.64 (m, 2H), 1.88-1.99 (m, 2H), 1.41 (s, 2H), 1.22-1.36 (m, 4H), 0.96-1.20 (m, 6H), 0.86 (s, 6H). MS (ESI) m/e 868.3 (M+H)⁺.

### 1.6. Synthesis of 6-[8-(1,3-benzothiazol-2-ylcarbamoyl)-3,4-dihydroisoquinolin-2(1H)-yl]-3-{1-[(3-{2-[(2,3-dihydroxypropyl)amino]ethoxy}-5,7-dimethyltricyclo[3.3.1.1^{3,7}]dec-1-yl)methyl]-5-methyl-1H-pyrazol-4-yl}pyridine-2-carboxylic Acid (Compound W2.07)

To a solution of Example 1.2.7 (30 mg) in dichloromethane (3 mL) was added 2,3-dihydroxypropanal (3.6 mg), and NaCNBH₃ on resin (200 mg). The mixture was stirred overnight, filtered, and the solvent was evaporated. The residue was dissolved in dimethyl sulfoxide/methanol (1:1, 3 mL) and purified by reverse phase HPLC using a Gilson system, eluting with 10-85% acetonitrile in 0.1% trifluoroacetic acid in water, to give the title compound. ¹H NMR (400 MHz, dimethyl sulfoxide-d₆) δ ppm 12.85 (s, 1H), 8.27 (s, 2H), 8.03 (d, 1H), 7.79 (d, 1H), 7.61 (t, 1H), 7.33-7.54 (m, 6H), 7.29 (s, 1H), 6.96 (d, 1H), 4.96 (s, 3H), 3.72-3.89 (m, 8H), 3.25-3.64 (m, 6H), 2.99-3.10 (m, 4H), 2.11 (s, 3H), 1.00-1.52 (m, 8H), 0.86 (s, 6H). MS (ESI) m/e 820.3 (M+H)⁺.

### 1.7. Synthesis of 2-({[4-({[2-({3-[(4-{6-[8-(1,3-benzothiazol-2-ylcarbamoyl)-3,4-dihydroisoquinolin-2(1H)-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl]amino}methyl)phenyl]sulfonyl}amino)-2-deoxy-beta-D-glucopyranose (Compound W2.08)

### 1.7.1. (2R,3S,4S,5R,6S)-6-(acetoxymethyl)-3-(4-formylphenylsulfonamido)tetrahydro-2H-pyran-2,4,5-triyl triacetate

4-Formylbenzene-1-sulfonyl chloride (100 mg) and (2S,3R,4R,5S,6R)-6-(acetoxymethyl)-3-aminotetrahydro-2H-pyran-2,4,5-triyl triacetate hydrochloride (563 mg) were added to 1,2-dichloroethane (4 mL). N,N-Diisopropylethylamine (0.51 mL) was added, and the solution was heated at 55° C. for three days. The solution was concentrated under reduced pressure and purified by flash column chromatography on silica gel, eluting with 70% ethyl acetate in heptanes. The solvent was evaporated under reduced pressure, and the material was dissolved in acetone (4 mL). Hydrochloric acid (1M, 4 mL) was added, and the solution was stirred at room temperature for 16 hours. The solution was then extracted with 70% ethyl acetate in heptanes (20 mL). The organic layer was washed with brine and dried over anhydrous sodium sulfate. After filtration, the solvent was evaporated under reduced pressure to provide the title compound. MS (ESI) m/e 514 (M+H)⁺.

### 1.7.2. (2R,3S,4S,5R,6S)-6-(acetoxymethyl)-3-(4-(((2-((3-((4-(6-(8-(benzo[d]thiazol-2-ylcarbamoyl)-3,4-dihydroisoquinolin-2(1H)-yl)-2-(tert-butoxycarbonyl)pyridin-3-yl)-5-methyl-1H-pyrazol-1-yl)methyl)-5,7-dimethyladamantan-1-yl)oxy)ethyl)amino)methyl)phenylsulfonamido)tetrahydro-2H-pyran-2,4,5-triyl Triacetate

The title compound was prepared by substituting Example 1.8.1 for Example 1.5.3 in Example 1.5.4. MS (ESI) m/e 1301 (M+H)⁺.

### 1.7.3. 2-({[4-({[2-({3-[(4-{6-[8-(1,3-benzothiazol-2-ylcarbamoyl)-3,4-dihydroisoquinolin-2(1H)-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl]amino}methyl)phenyl]sulfonyl}amino)-2-deoxy-beta-D-glucopyranose

The title compound was prepared by substituting Example 1.8.2 for Example 1.5.4 in Example 1.5.5. ¹H NMR (400 MHz, dimethyl sulfoxide-d₆) δ ppm 12.86 (bs, 1H), 8.87 (bs, 2H), 8.04 (d, 1H), 7.91 (d, 2H), 7.79 (d, 1H), 7.70-7.55 (m, 3H), 7.52-7.42 (m, 3H), 7.39-7.33 (m, 2H), 7.29 (m, 1H), 6.96 (d, 1H), 4.96 (bs, 2H), 4.85 (dd, 1H), 4.62-4.52 (m, 2H), 4.32 (m, 2H), 3.89 (t, 2H), 3.83 (s, 2H), 3.70-3.35 (m, 10H), 3.02 (m, 4H), 2.91 (m, 1H), 2.10 (s, 3H), 1.44 (bs, 2H), 1.37-1.22 (m, 4H), 1.18-0.98 (m, 6H), 0.93-0.82 (m, 6H). MS (ESI) m/e 1075 (M+H)⁺.

### 1.8. Synthesis of 8-(1,3-benzothiazol-2-ylcarbamoyl)-2-{6-carboxy-5-[1-({3-[2-({2-[1-(beta-D-glucopyranuronosyl)-1H-1,2,3-triazol-4-yl]ethyl}amino)ethoxy]-5,7-dimethyltricyclo[3.3.1.1^{3,7}]dec-1-yl}methyl)-5-methyl-1H-pyrazol-4-yl]pyridin-2-yl}-1,2,3,4-tetrahydroisoquinoline (Compound W2.09)

### 1.8.1. (2R,3R,4S,5S,6S)-2-(4-(2-hydroxyethyl)-1H-1,2,3-triazol-1-yl)-6-(methoxycarbonyl)tetrahydro-2H-pyran-3,4,5-triyl Triacetate

To a solution of (2R,3R,4S,5S,6S)-2-azido-6-(methoxycarbonyl)tetrahydro-2H-pyran-3,4,5-triyl triacetate (720 mg) in t-butanol (8 mL) and water (4 mL) was added but-3-yn-1-ol (140 mg), copper(II) sulfate pentahydrate (5.0 mg) and sodium ascorbate (40 mg). The mixture was stirred 20 minutes at 100° C. under microwave conditions (Biotage Initiator). The reaction mixture was diluted with ethyl acetate (300 mL), washed with water and brine, and dried over sodium sulfate. Filtration and evaporation of the solvent provided the title compound. MS (ESI) m/e 430.2 (M+H)⁺.

### 1.8.2. (2S,3S,4S,5R,6R)-2-(methoxycarbonyl)-6-(4-(2-oxoethyl)-1H-1,2,3-triazol-1-yl)tetrahydro-2H-pyran-3,4,5-triyl Triacetate

To a solution of dimethyl sulfoxide (0.5 mL) in dichloromethane (10 mL) at −78° C. was added oxalyl chloride (0.2 mL). The mixture was stirred 20 minutes at −78° C., and a solution of (2R,3R,4S,5S,6S)-2-(4-(2-hydroxyethyl)-1H-1,2,3-triazol-1-yl)-6-(methoxycarbonyl)tetrahydro-2H-pyran-3,4,5-triyl triacetate (233 mg) in dichloromethane (10 mL) was added through a syringe. After 20 minutes, triethylamine (1 mL) was added to the mixture, and the mixture was stirred for 30 minutes while the temperature was allowed to rise to room temperature. The reaction mixture was diluted with ethyl acetate (300 mL), washed with water and brine, and dried over sodium sulfate. Filtration and evaporation of the solvent gave the crude product, which was used in the next reaction without further purification. MS (ESI) m/e 429.2 (M+H)⁺.

### 1.8.3. 8-(1,3-benzothiazol-2-ylcarbamoyl)-2-{6-carboxy-5-[1-({3-[2-({2-[1-(beta-D-glucopyranuronosyl)-1H-1,2,3-triazol-4-yl]ethyl}amino)ethoxy]-5,7-dimethyltricyclo[3.3.1.1^{3,7}]dec-1-yl}methyl)-5-methyl-1H-pyrazol-4-yl]pyridin-2-yl}-1,2,3,4-tetrahydroisoquinoline

To a solution of Example 1.3.1 (150 mg) in dichloromethane (10 mL) was added Example 1.9.2 (86 mg) and NaBH₃CN on resin (2.49 mmol/g, 200 mg), and the mixture was stirred overnight. The reaction mixture was then filtered and concentrated. The residue was dissolved in tetrahydrofuran/methanol/H₂O (2:1:1, 12 mL) and lithium hydroxide monohydrate (50 mg) was added. The mixture was stirred overnight. The mixture was concentrated, and the residue was purified by reverse phase HPLC using a Gilson system, eluting with 10-85% acetonitrile in 0.1% trifluoroacetic acid in water, to provide the title compound. ¹H NMR (400 MHz, dimethyl sulfoxide-d₆) δ ppm 12.84 (s, 1H), 8.48 (s, 2H), 8.20 (s, 1H), 8.03 (d, 1H), 7.79 (d, 1H), 7.62 (d, 1H), 7.32-7.53 (m, 5H), 7.29 (s, 1H), 6.96 (d, 1H), 5.66 (d, 1H), 4.96 (s, 2H), 4.00 (d, 1H), 3.76-3.92 (m, 6H), 3.22-3.26 (m, 2H), 2.96-3.15 (m, 8H), 2.10 (s, 3H), 0.99-1.52 (m, 14H), 0.87 (s, 6H). MS (ESI) m/e 1028.3 (M+H)⁺.

### 1.9. Synthesis of 3-[1-({3-[2-(2-{[4-(beta-D-allopyranosyloxy)benzyl]amino}ethoxy)ethoxy]-5,7-dimethyltricyclo[3.3.1.1^{3,7}]dec-1-yl}methyl)-5-methyl-1H-pyrazol-4-yl]-6-[8-(1,3-benzothiazol-2-ylcarbamoyl)-3,4-dihydroisoquinolin-2(1H)-yl]pyridine-2-carboxylic Acid (Compound W2.10)

### 1.9.1. 2-(2-((3-((1H-pyrazol-1-yl)methyl)-5,7-dimethyladamantan-1-yl)oxy)ethoxy)ethanol

The title compound was prepared as in Example 1.1.4 by substituting ethane-1,2-diol with 2,2′-oxydiethanol. MS (ESI) m/e 349.2 (M+H)⁺.

### 1.9.2. 2-(2-((3,5-dimethyl-7-((5-methyl-1H-pyrazol-1-yl)methyl)adamantan-1-yl)oxy)ethoxy)ethanol

The title compound was prepared as in Example 1.1.5 by substituting Example 1.1.4 with Example 1.10.1. MS (ESI) m/e 363.3 (M+H)⁺.

### 1.9.3. 2-(2-((3-((4-iodo-5-methyl-1H-pyrazol-1-yl)methyl)-5,7-dimethyladamantan-1-yl)oxy)ethoxy)ethanol

The title compound was prepared as in Example 1.1.6 by substituting Example 1.1.5 with Example 1.10.2. MS (ESI) m/e 489.2 (M+H)⁺.

### 1.9.4. 2-(2-((3-((4-iodo-5-methyl-1H-pyrazol-1-yl)methyl)-5,7-dimethyladamantan-1-yl)oxy)ethoxy)ethyl Methanesulfonate

To a cooled solution of Example 1.10.3 (6.16 g) in dichloromethane (100 mL) was added triethylamine (4.21 g) followed by methanesulfonyl chloride (1.6 g), and the mixture was stirred at room temperature for 1.5 hours. The reaction mixture was then diluted with ethyl acetate (600 mL) and washed with water and brine. After drying over sodium sulfate, the solution was filtered and concentrated, and the residue was used in the next reaction without further purification. MS (ESI) m/e 567.2 (M+H)⁺.

### 1.9.5. 2-(2-((3-((4-iodo-5-methyl-1H-pyrazol-1-yl)methyl)-5,7-dimethyladamantan-1-yl)oxy)ethoxy)ethanamine

A solution of Example 1.10.4 (2.5 g) in 7N ammonia in methanol (15 mL) was stirred at 100° C. for 20 minutes under microwave conditions (Biotage Initiator). The reaction mixture was concentrated under vacuum, and the residue was diluted with ethyl acetate (400 mL) and washed with aqueous NaHCO₃, water and brine. After drying over sodium sulfate, the solution was filtered and concentrated, and the residue was used in the next reaction without further purification. MS (ESI) m/e 488.2 (M+H)⁺.

### 1.9.6. tert-butyl (2-(2-((3-((4-iodo-5-methyl-1H-pyrazol-1-yl)methyl)-5,7-dimethyladamantan-1-yl)oxy)ethoxy)ethyl)carbamate

To a solution of Example 1.10.5 (2.2 g) in tetrahydrofuran (30 mL) was added di-tert-butyl dicarbonate (1.26 g) and 4-dimethylaminopyridine (100 mg). The mixture was stirred at room temperature for 1.5 hours and was diluted with ethyl acetate (300 mL). The solution was washed with saturated aqueous NaHCO₃, water (60 mL) and brine (60 mL). The organic layer was dried with sodium sulfate, filtered and concentrated. The residue was purified by silica gel chromatography, eluting with 20% ethyl acetate in dichloromethane, to give the title compound. MS (ESI) m/e 588.2 (M+H)⁺.

### 1.9.7. Methyl 2-(6-(tert-butoxycarbonyl)-5-(1-((3-(2-(2-((tert-butoxycarbonyl)amino)ethoxy)ethoxy)-5,7-dimethyladamantan-1-yl)methyl)-5-methyl-1H-pyrazol-4-yl)pyridin-2-yl)-1,2,3,4-tetrahydroisoquinoline-8-carboxylate

The title compound was prepared as in Example 1.2.2 by substituting Example 1.1.6 with Example 1.10.6. MS (ESI) m/e 828.5 (M+H)⁺.

### 1.9.8. 2-(6-(tert-butoxycarbonyl)-5-(1-((3-(2-(2-((tert-butoxycarbonyl)amino)ethoxy)ethoxy)-5,7-dimethyladamantan-1-yl)methyl)-5-methyl-1H-pyrazol-4-yl)pyridin-2-yl)-1,2,3,4-tetrahydroisoquinoline-8-carboxylic Acid

The title compound was prepared as in Example 1.2.5 by substituting Example 1.2.4 with Example 1.10.7. MS (ESI) m/e 814.5 (M+H)⁺.

### 1.9.9. tert-butyl 6-(8-(benzo[d]thiazol-2-ylcarbamoyl)-3,4-dihydroisoquinolin-2(1H)-yl)-3-(1-((3-(2-(2-((tert-butoxycarbonyl)amino)ethoxy)ethoxy)-5,7-dimethyladamantan-1-yl)methyl)-5-methyl-1H-pyrazol-4-yl)picolinate

The title compound was prepared as in Example 1.2.6 by substituting Example 1.2.5 with Example 1.10.8. MS (ESI) m/e 946.2 (M+H)⁺.

### 1.9.10.3-(1-((3-(2-(2-aminoethoxy)ethoxy)-5,7-dimethyladamantan-1-yl)methyl)-5-methyl-1H-pyrazol-4-yl)-6-(8-(benzo[d]thiazol-2-ylcarbamoyl)-3,4-dihydroisoquinolin-2(1H)-yl)picolinic Acid

The title compound was prepared as in Example 1.1.17 by substituting Example 1.1.16 with Example 1.10.9.

### 1.9.11. 3-[1-({3-[2-(2-{[4-(beta-D-allopyranosyloxy)benzyl]amino}ethoxy)ethoxy]-5,7-dimethyltricyclo[3.3.1.1^{3,7}]dec-1-yl}methyl)-5-methyl-1H-pyrazol-4-yl]-6-[8-(1,3-benzothiazol-2-ylcarbamoyl)-3,4-dihydroisoquinolin-2(1H)-yl]pyridine-2-carboxylic Acid

To a solution of Example 1.10.10 (88 mg) and triethylamine (0.04 mL) in dichloromethane (1.5 mL) was added 4-(((2S,3R,4R,5S,6R)-3,4,5-trihydroxy-6-(hydroxymethyl)tetrahydro-2H-pyran-2-yl)oxy)benzaldehyde (27.7 mg), methanol (1 mL), MP-CNBH₃ (2.49 mmol/g, 117 mg) and acetic acid (18 μL). The reaction mixture was stirred overnight. The reaction was filtered, and the filtrate was concentrated. The residue was purified by purified by reverse phase chromatography (C18 column), eluting with 20-60% acetonitrile in water containing 0.1% v/v trifluoroacetic acid, to provide the title compound. ¹H NMR (400 MHz, dimethyl sulfoxide-d₆) δ ppm 7.99 (d, 1H), 7.77 (d, 1H), 7.60 (d, 1H), 7.40-7.50 (m, 2H), 7.29-7.39 (m, 6H), 6.96 (d, 2H), 6.76 (d, 1H), 5.11 (d, 2H), 4.92 (s, 2H), 3.83-3.96 (m, 4H), 3.77 (s, 2H), 3.60-3.72 (m, 4H), 3.01 (d, 2H), 2.80 (t, 2H), 2.09 (s, 3H), 0.98-1.32 (m, 14H), 0.82 (s, 6H). MS (ESI) m/e 1058.3 (M+H)⁺.

### 1.10. Synthesis of 6-[8-(1,3-benzothiazol-2-ylcarbamoyl)-3,4-dihydroisoquinolin-2(1H)-yl]-3-(1-{[3,5-dimethyl-7-(2-{2-[(2-sulfoethyl)amino]ethoxy}ethoxy)tricyclo[3.3.1.1^{3,7}]dec-1-yl]methyl}-5-methyl-1H-pyrazol-4-yl)pyridine-2-carboxylic Acid (Compound W2.11)

### 1.10.1. tert-butyl 3-(1-((3-(2-(2-aminoethoxy)ethoxy)-5,7-dimethyladamantan-1-yl)methyl)-5-methyl-1H-pyrazol-4-yl)-6-(8-(benzo[d]thiazol-2-ylcarbamoyl)-3,4-dihydroisoquinolin-2(1H)-yl)picolinate

Example 1.10.9 (6.8 g) was dissolved in 50% trifluoroacetic acid in dichloromethane (10 mL) and stirred for 20 minutes, and the solvents were removed under vacuum. The residue was purified by reverse phase chromatography, eluting with 20-80% acetonitrile in water containing 0.1% trifluoroacetic acid, to provide the title compound. MS (ESI) m/e 790.2 (M+H)⁺.

### 1.10.2. tert-butyl 6-(8-(benzo[d]thiazol-2-ylcarbamoyl)-3,4-dihydroisoquinolin-2(1H)-yl)-3-(1-((3,5-dimethyl-7-(2-(2-((2-(phenoxysulfonyl)ethyl)amino)ethoxy)ethoxy)adamantan-1-yl)methyl)-5-methyl-1H-pyrazol-4-yl)picolinate

To a solution of Example 1.11.1 (200 mg) and N,N-diisopropylethylamine (146 μL) in tetrahydrofuran (3 mL) at 0° C. was added phenyl ethanesulfonate (46 mg). The reaction mixture was stirred at 0° C. for 30 minutes, gradually warmed to room temperature, stirred overnight and concentrated to provide the title compound.

### 1.10.3. 6-(8-(benzo[d]thiazol-2-ylcarbamoyl)-3,4-dihydroisoquinolin-2(1H)-yl)-3-(1-((3,5-dimethyl-7-(2-(2-((2-(phenoxysulfonyl)ethyl)amino)ethoxy)ethoxy)adamantan-1-yl)methyl)-5-methyl-1H-pyrazol-4-yl)picolinic Acid

A solution of Example 1.11.2 (100 mg) in dichloromethane (5 mL) was treated with trifluoroacetic acid (2.5 mL) overnight and concentrated to provide the title compound. MS (APCI) m/e 974.9 (M+H)⁺.

### 1.10.4. 6-[8-(1,3-benzothiazol-2-ylcarbamoyl)-3,4-dihydroisoquinolin-2(1H)-yl]-3-(1-{[3,5-dimethyl-7-(2-{2-[(2-sulfoethyl)amino]ethoxy}ethoxy)tricyclo[3.3.1.1^{3,7}]dec-1-yl]methyl}-5-methyl-1H-pyrazol-4-yl)pyridine-2-carboxylic Acid

To a solution of Example 1.11.3 (195 mg) in tetrahydrofuran (3 mL) and methanol (2 mL) was slowly added 1M sodium hydroxide aqueous solution (2 mL). The mixture was stirred overnight, and NaOH pellets (0.5 g) were added. The resulting mixture was heated at 40° C. for 3 hours, cooled and concentrated. The concentrate was purified by reverse phase chromatography (C18 column), eluting with 10-70% acetonitrile in 10 mM aqueous NH₄OAc solution, to provide the title compound. ¹H NMR (400 MHz, dimethyl sulfoxide-d₆) δ ppm 8.04 (d, 1H), 7.79 (d, 1H), 7.61 (d, 1H), 7.41-7.51 (m, 3H), 7.32-7.39 (m, 2H), 7.29 (s, 1H), 6.88 (d, 1H), 4.93 (s, 2H), 3.89 (t, 2H), 3.81 (s, 2H), 3.60-3.66 (m, 4H), 3.13-3.19 (m, 2H), 3.05-3.10 (m, 2H), 3.01 (t, 2H), 2.79 (t, 2H), 2.11 (s, 3H), 1.34 (s, 2H), 1.26 (s, 4H), 0.96-1.22 (m, 6H), 0.85 (s, 6H). MS (ESI) m/e 898.2 (M+H)⁺.

### 1.11. Synthesis of 6-[8-(1,3-benzothiazol-2-ylcarbamoyl)-3,4-dihydroisoquinolin-2(1H)-yl]-3-{1-[(3,5-dimethyl-7-{2-[(2-phosphonoethyl)amino]ethoxy}tricyclo[3.3.1.1^{3,7}]dec-1-yl)methyl]-5-methyl-1H-pyrazol-4-yl}pyridine-2-carboxylic acid (Compound W2.12)

### 1.11.1. tert-butyl 6-(8-(benzo[d]thiazol-2-ylcarbamoyl)-3,4-dihydroisoquinolin-2(1H)-yl)-3-(1-((3-(2-((2-(diethoxyphosphoryl)ethyl)amino)ethoxy)-5,7-dimethyladamantan-1-yl)methyl)-5-methyl-1H-pyrazol-4-yl)picolinate

To a solution of Example 1.2.7 (307 mg) in tetrahydrofuran (5 mL) was added diethyl vinylphosphonate (176 mg) in water (2 mL). The reaction mixture was stirred at 70° C. for 3 days, and a few drops of acetic acid were added. The mixture was purified by reverse phase chromatography (C18 column), eluting with 10-70% acetonitrile in water containing 0.1% v/v trifluoroacetic acid, to provide the title compound. MS (APCI) m/e 966.8 (M+H)⁺.

### 1.11.2. 6-[8-(1,3-benzothiazol-2-ylcarbamoyl)-3,4-dihydroisoquinolin-2(1H)-yl]-3-{1-[(3,5-dimethyl-7-{2-[(2-phosphonoethyl)amino]ethoxy}tricyclo[3.3.1.1^{3,7}]dec-1-yl)methyl]-5-methyl-1H-pyrazol-4-yl}pyridine-2-carboxylic Acid

To a solution of Example 1.12.1 (170 mg) in dichloromethane (2.5 mL) was added bromotrimethylsilane (82 μL) and allyltrimethylsilane (50.4 μL). The reaction mixture was stirred overnight and water (0.02 mL) was added. The resulting mixture was stirred overnight and concentrated. The residue was purified by reverse phase chromatography (C18 column), eluting with 20-60% acetonitrile in water containing 0.1% trifluoroacetic acid, to provide the title compound. ¹H NMR (500 MHz, dimethyl sulfoxide-d₆) δ ppm 8.35 (s, 2H), 8.03 (d, 1H), 7.79 (d, 1H), 7.62 (d, 1H), 7.41-7.53 (m, 3H), 7.33-7.40 (m, 2H), 7.29 (s, 1H), 6.96 (d, 1H), 4.96 (s, 2H), 3.89 (t, 2H), 3.83 (s, 2H), 3.09 (s, 4H), 3.01 (t, 2H), 2.10 (s, 3H), 1.85-2.00 (m, 2H), 1.43 (s, 2H), 1.19-1.37 (m, 4H), 1.14 (s, 6H), 0.87 (s, 6H). MS (APCI) m/e 854.4 (M+H)⁺.

### 1.12. Synthesis of 6-[8-(1,3-benzothiazol-2-ylcarbamoyl)-3,4-dihydroisoquinolin-2(1H)-yl]-3-{1-[(3,5-dimethyl-7-{2-[methyl(3-sulfo-L-alanyl)amino]ethoxy}tricyclo[3.3.1.1^{3,7}]dec-1-yl)methyl]-5-methyl-1H-pyrazol-4-yl}pyridine-2-carboxylic Acid (Compound W2.13)

### 1.12.1. 2-({3-[(4-iodo-5-methyl-1H-pyrazol-1-yl)methyl]-5,7-dimethyltricyclo[3.3.1.1^{3,7}]dec-1-yl}oxy)ethyl Methanesulfonate

To a cooled solution of Example 1.1.6 (6.16 g) in dichloromethane (100 mL) was added triethylamine (4.21 g) followed by methanesulfonyl chloride (1.6 g), and the mixture was stirred at room temperature for 1.5 hours. The reaction mixture was diluted with ethyl acetate (600 mL) and washed with water and brine. After drying over sodium sulfate, the solution was filtered and concentrated, and the residue was used in the next reaction without further purification. MS (ESI) m/e 523.4 (M+H)⁺.

### 1.12.2. 1-({3,5-dimethyl-7-[2-(methylamino)ethoxy]tricyclo[3.3.1.1^{3,7}]dec-1-yl}methyl)-4-iodo-5-methyl-1H-pyrazole

A solution of Example 1.13.1 (2.5 g) in 2M methylamine in methanol (15 mL) was stirred at 100° C. for 20 minutes under microwave conditions (Biotage Initiator). The reaction mixture was concentrated under vacuum, and the residue was diluted with ethyl acetate (400 mL) and washed with aqueous NaHCO₃, water and brine. After drying over sodium sulfate, the solution was filtered and concentrated, and the residue was used in the next reaction without further purification. MS (ESI) m/e 458.4 (M+H)⁺.

### 1.12.3. tert-butyl [2-({3-[(4-iodo-5-methyl-1H-pyrazol-1-yl)methyl]-5,7-dimethyltricyclo[3.3.1.1^{3,7}]dec-1-yl}oxy)ethyl]methylcarbamate

To a solution of Example 1.13.2 (2.2 g) in tetrahydrofuran (30 mL) was added di-tert-butyl dicarbonate (1.26 g) and a catalytic amount of 4-dimethylaminopyridine. The mixture was stirred at room temperature for 1.5 hours and diluted with ethyl acetate (300 mL). The solution was washed with saturated aqueous NaHCO₃, water (60 mL) and brine (60 mL). The organic layer was dried with sodium sulfate, filtered and concentrated. The residue was purified by silica gel chromatography, eluting with 20% ethyl acetate in dichloromethane, to give the title compound. MS (ESI) m/e 558.5 (M+H)⁺.

### 1.12.4. Methyl 2-(6-(tert-butoxycarbonyl)-5-(1-((3-(2-((tert-butoxycarbonyl)(methyl)amino)ethoxy)-5,7-dimethyltricyclo[3.3.1.1^{3,7}]dec-1-yl)methyl)-5-methyl-1H-pyrazol-4-yl)pyridin-2-yl)-1,2,3,4-tetrahydroisoquinoline-8-carboxylate

To a solution of Example 1.2.1 (4.94 g) in tetrahydrofuran (60 mL) and water (20 mL) was added Example 1.13.3 (5.57 g), 1,3,5,7-tetramethyl-8-tetradecyl-2,4,6-trioxa-8-phosphaadamantane (412 mg), tris(dibenzylideneacetone)dipalladium(0) (457 mg), and K₃PO₄ (11 g), and the mixture was stirred at reflux for 24 hours. The reaction mixture was cooled and diluted with ethyl acetate (500 mL), washed with water and brine. The organic layer was dried over sodium sulfate, filtered and concentrated under reduced pressure. Purification of the residue by silica gel chromatography, eluting with 20% ethyl acetate in heptane, provided the title compound. MS (ESI) m/e 799.1 (M+H)⁺.

### 1.12.5. 2-(6-(tert-butoxycarbonyl)-5-(1-((3-(2-((tert-butoxycarbonyl)(methyl)amino)ethoxy)-5,7-dimethyltricyclo[3.3.1.1^{3,7}]dec-1-yl)methyl)-5-methyl-1H-pyrazol-4-yl)pyridin-2-yl)-1,2,3,4-tetrahydroisoquinoline-8-carboxylic Acid

To a solution of Example 1.13.4 (10 g) in tetrahydrofuran (60 mL), methanol (30 mL) and water (30 mL) was added lithium hydroxide monohydrate (1.2 g), and the mixture was stirred at room temperature for 24 hours. The reaction mixture was neutralized with 2% aqueous HCl and concentrated under vacuum. The residue was diluted with ethyl acetate (800 mL) and washed with water and brine, and dried over sodium sulfate. Filtration and evaporation of the solvent provided the title compound. MS (ESI) m/e 785.1 (M+H)⁺.

### 1.12.6. tert-butyl 6-[8-(1,3-benzothiazol-2-ylcarbamoyl)-3,4-dihydroisoquinolin-2(1H)-yl]-3-{1-[(3-{2-[(tert-butoxycarbonyl)(methyl)amino]ethoxy}-5,7-dimethyltricyclo[3.3.1.1^{3,7}]dec-1-yl)methyl]-5-methyl-1H-pyrazol-4-yl}pyridine-2-carboxylate

To a solution of Example 1.13.5 (10 g) in N,N-dimethylformamide (20 mL) was added benzo[d]thiazol-2-amine (3.24 g), fluoro-N,N,N′,N′-tetramethylformamidinium hexafluorophosphate (5.69 g) and N,N-diisopropylethylamine (5.57 g), and the mixture was stirred at 60° C. for 3 hours. The reaction mixture was diluted with ethyl acetate (800 mL) and washed with water and brine, and dried over sodium sulfate. Filtration and evaporation of the solvent and silica gel purification of the residue, eluting with 20% ethyl acetate in dichloromethane, provided the title compound. MS (ESI) m/e 915.5 (M+H)⁺.

### 1.12.7. 6-[8-(1,3-benzothiazol-2-ylcarbamoyl)-3,4-dihydroisoquinolin-2(1H)-yl]-3-[1-({3,5-dimethyl-7-[2-(methylamino)ethoxy]tricyclo[3.3.1.1^{3,7}]dec-1-yl}methyl)-5-methyl-1H-pyrazol-4-yl]pyridine-2-carboxylic Acid

To a solution of Example 1.13.6 (5 g) in dichloromethane (20 mL) was added trifluoroacetic acid (10 mL), and the mixture was stirred overnight. The solvent was evaporated under vacuum, and the residue was dissolved in dimethyl sulfoxide/methanol (1:1, 10 mL). The mixture was purified by reverse phase chromatography using an Analogix system and a C18 column (300 g), and eluting with 10-85% acetonitrile and 0.1% trifluoroacetic acid in water, to give the title compound.

### 1.12.8. 6-[8-(1,3-benzothiazol-2-ylcarbamoyl)-3,4-dihydroisoquinolin-2(1H)-yl]-3-{1-[(3,5-dimethyl-7-{2-[methyl(3-sulfo-L-alanyl)amino]ethoxy}tricyclo[3.3.1.1^{3,7}]dec-1-yl)methyl]-5-methyl-1H-pyrazol-4-yl}pyridine-2-carboxylic Acid

A solution of (R)-2-((((9H-fluoren-9-yl)methoxy)carbonyl)amino)-3-sulfopropanoic acid (0.020 g), N,N-diisopropylethylamine (0.045 mL) and O-(7-azabenzotriazol-1-yl)-N,N,N′,N′-tetramethyluronium hexafluorophosphate (HATU, 0.020 g) were stirred together in N,N-dimethylformamide (0.75 mL) at room temperature. After stirring for 30 minutes, Example 1.13.7 (0.039 g) was added, and the reaction stirred for an additional 1 hour. Diethylamine (0.027 mL) was added to the reaction and stirring was continued for 3 hours. The reaction was diluted with water (0.75 mL) and N,N-dimethylformamide (1 mL), neutralized with trifluoroacetic acid (0.039 mL) and purified by reverse phase HPLC using a Gilson system, eluting with 20-80% acetonitrile in water containing 0.1% v/v trifluoroacetic acid. The desired fractions were combined and freeze-dried to provide the title compound. ¹H NMR (400 MHz, dimethyl sulfoxide-d₆) δ ppm 12.89 (s, 1H), 8.11-8.02 (m, 4H), 7.84 (d, 1H), 7.66 (d, 1H), 7.60-7.45 (m, 3H), 7.45-7.36 (m, 2H), 7.34 (d, 1H), 7.00 (dd, 1H), 5.00 (s, 2H), 4.57-4.40 (m, 1H), 3.93 (t, 2H), 3.90-3.84 (m, 2H), 3.58-3.43 (m, 2H), 3.41-3.21 (m, 2H), 3.18-3.02 (m, 3H), 2.95-2.85 (m, 2H), 2.76 (td, 2H), 2.14 (d, 3H), 1.51-0.85 (m, 18H). MS (ESI) m/e 911.2 (M+H)⁺.

### 1.13. Synthesis of 6-[8-(1,3-benzothiazol-2-ylcarbamoyl)-3,4-dihydroisoquinolin-2(1H)-yl]-3-{1-[(3,5-dimethyl-7-{2-[(3-phosphonopropyl)amino]ethoxy}tricyclo[3.3.1.1^{3,7}]dec-1-yl)methyl]-5-methyl-1H-pyrazol-4-yl}pyridine-2-carboxylic acid (Compound W2.14)

### 1.13.1. di-tert-butyl (3-hydroxypropyl)phosphonate

NaH (60% in mineral oil, 400 mg) was added to di-tert-butylphosphonate (1.93 g) in N,N-dimethylformamide (30 mL), and the reaction was stirred at room temperature for 30 minutes. (3-Bromopropoxy)(tert-butyl)dimethylsilane (2.1 g) was added, and the reaction was stirred overnight. The mixture was diluted with diethyl ether (300 mL), and the solution was washed three times with water, and brine, then dried over sodium sulfate, filtered, and concentrated. The residue was dissolved in 20 mL tetrahydrofuran, and tetrabutyl ammonium fluoride (TBAF, 1M in tetrahydrofuran, 9 mL) was added. The solution was stirred for 20 minutes, and then pH 7 buffer (50 mL) was added. The mixture was taken up in diethyl ether, and separated, and the organic layer was washed with brine, and then concentrated. The crude product was chromatographed on silica gel using 10-100% ethyl acetate in heptanes, followed by 5% methanol in ethyl acetate to provide the title compound.

### 1.13.2. di-tert-butyl (3-oxopropyl)phosphonate

Example 1.14.1 (200 mg) and Dess-Martin periodinane (370 mg) were stirred in dichloromethane (5 mL) for 2 hours. The mixture was taken up in ethyl acetate, and washed twice with 1M aqueous NaOH solution, and brine, and then concentrated. The crude product was chromatographed on silica gel, using 50-100% ethyl acetate in heptanes followed by 10% methanol in ethyl acetate, to provide the title compound.

### 1.13.3. tert-butyl 6-(8-(benzo[d]thiazol-2-ylcarbamoyl)-3,4-dihydroisoquinolin-2(1H)-yl)-3-(1-((3-(2-((3-(diethoxyphosphoryl)propyl)amino)ethoxy)-5,7-dimethyladamantan-1-yl)methyl)-5-methyl-1H-pyrazol-4-yl)picolinate

The title compound was prepared as described in Example 1.10.11, replacing Example 1.10.10 and 4-(((2S,3R,4R,5S,6R)-3,4,5-trihydroxy-6-(hydroxymethyl)tetrahydro-2H-pyran-2-yl)oxy)benzaldehyde with Example 1.2.7 and Example 1.14.2, respectively. MS (APCI) m/e 980.9 (M+H)⁺.

### 1.13.4. 6-[8-(1,3-benzothiazol-2-ylcarbamoyl)-3,4-dihydroisoquinolin-2(1H)-yl]-3-{1-[(3,5-dimethyl-7-{2-[(3-phosphonopropyl)amino]ethoxy}tricyclo[3.3.1.1^{3,7}]dec-1-yl)methyl]-5-methyl-1H-pyrazol-4-yl}pyridine-2-carboxylic Acid

The title compound was prepared as described in Example 1.12.2, replacing Example 1.12.1 with Example 1.14.3. ¹H NMR (400 MHz, dimethyl sulfoxide-d₆) δ ppm 8.37 (s, 2H), 8.03 (d, 1H), 7.79 (d, 1H), 7.62 (d, 1H), 7.42-7.53 (m, 3H), 7.33-7.40 (m, 2H), 7.29 (s, 1H), 6.96 (d, 1H), 4.96 (s, 2H), 3.86-3.93 (m, 2H), 3.52-3.59 (m, 2H), 2.93-3.06 (m, 6H), 2.10 (s, 3H), 1.71-1.89 (m, 2H), 1.53-1.65 (m, 2H), 1.43 (s, 2H), 1.23-1.37 (m, 4H), 0.96-1.19 (m, 6H), 0.87 (s, 6H). MS (APCI) m/e 868.3 (M+H)⁺.

### 1.14. Synthesis of 6-[8-(1,3-benzothiazol-2-ylcarbamoyl)-3,4-dihydroisoquinolin-2(1H)-yl]-3-{1-[(3,5-dimethyl-7-{2-[(3-sulfo-L-alanyl)amino]ethoxy}tricyclo[3.3.1.1^{3,7}]dec-1-yl)methyl]-5-methyl-1H-pyrazol-4-yl}pyridine-2-carboxylic Acid (Compound W2.15)

A solution of (R)-2-((((9H-fluoren-9-yl)methoxy)carbonyl)amino)-3-sulfopropanoic acid (0.050 g) and O-(7-azabenzotriazol-1-yl)-N,N,N′,N′-tetramethyluronium hexafluorophosphate (0.049 g) were dissolved in N,N-dimethylformamide (1 mL) and N,N-diisopropylethylamine (0.102 mL) was added. After stirring for 15 minutes, Example 1.3.1 (0.100 g) was added, and the reaction stirred for an additional 3 hours. Diethylamine (0.061 mL) was added to the reaction and stirring was continued overnight. The reaction was neutralized with 2,2,2-trifluoroacetic acid (0.090 mL) and diluted with N,N-dimethylformamide (1 mL) and water (1 mL). The mixture was purified by reverse phase HPLC using a Gilson system, eluting with 20-80% acetonitrile in water containing 0.1% v/v trifluoroacetic acid. The desired fractions were combined and freeze-dried to provide the title compound. ¹H NMR (500 MHz, dimethyl sulfoxide-d₆) δ ppm 12.86 (s, 1H), 8.63 (t, 1H), 8.15-8.01 (m, 4H), 7.79 (d, 1H), 7.62 (d, 1H), 7.56-7.41 (m, 3H), 7.40-7.33 (m, 2H), 7.30 (s, 1H), 6.96 (d, 1H), 4.96 (s, 2H), 4.08-3.97 (m, 1H), 3.89 (t, 2H), 3.82 (s, 2H), 3.42-3.31 (m, 2H), 3.28-3.17 (m, 1H), 3.16-3.06 (m, 1H), 3.01 (t, 2H), 2.97 (dd, 1H), 2.76 (dd, 1H), 2.10 (s, 3H), 1.39 (s, 2H), 1.32-1.20 (m, 4H), 1.19-1.07 (m, 4H), 1.07-0.95 (m, 2H), 0.85 (s, 6H). MS (ESI) m/e 897.2 (M+H)⁺.

### 1.15. Synthesis of 6-[8-(1,3-benzothiazol-2-ylcarbamoyl)-3,4-dihydroisoquinolin-2(1H)-yl]-3-(1-{[3,5-dimethyl-7-(2-{2-[(3-phosphonopropyl)amino]ethoxy}ethoxy)tricyclo[3.3.1.1^{3,7}]dec-1-yl]methyl}-5-methyl-1H-pyrazol-4-yl)pyridine-2-carboxylic Acid (Compound W2.16)

### 1.15.1. tert-butyl 6-(8-(benzo[d]thiazol-2-ylcarbamoyl)-3,4-dihydroisoquinolin-2(1H)-yl)-3-(1-((3-(2-(2-((3-(di-tert-butoxyphosphoryl)propyl)amino)ethoxy)ethoxy)-5,7-dimethyladamantan-1-yl)methyl)-5-methyl-1H-pyrazol-4-yl)picolinate

Example 1.10.10 (338 mg) and Example 1.14.2 (120 mg) were dissolved in ethanol (20 mL), and the solution was concentrated. The residue was again taken up in ethanol (20 mL) and concentrated. The residue was then dissolved in dichloromethane (10 mL) and to this was added sodium triacetoxyborohydride (119 mg), and the reaction was stirred overnight. The crude mixture was chromatographed on silica gel, using 1% triethylamine in 95:5 ethyl acetate/methanol, to provide the title compound. MS (ESI) 1080.3 (M+H)⁺.

### 1.15.2. 6-[8-(1,3-benzothiazol-2-ylcarbamoyl)-3,4-dihydroisoquinolin-2(1H)-yl]-3-(1-{[3,5-dimethyl-7-(2-{2-[(3-phosphonopropyl)amino]ethoxy}ethoxy)tricyclo[3.3.1.1^{3,7}]dec-1-yl]methyl}-5-methyl-1H-pyrazol-4-yl)pyridine-2-carboxylic Acid

Example 1.16.1 (22 mg) was stirred in dichloromethane (3 mL) and trifluoroacetic acid (3 mL) for 2 days. The mixture was concentrated and chromatographed via reverse phase on a Biotage Isolera One system using a 40 g C18 column and eluting with 10-90% acetonitrile in 0.1% trifluoroacetic acid/water, to provide the title compound as a trifluoroacetic acid salt. ¹H NMR (400 MHz, dimethyl sulfoxide-d₆) δ ppm 8.62 (bs, 1H), 8.10 (d, 1H), 7.86 (d, 1H), 7.68 (d, 1H), 7.57 (d, 1H), 7.54 (dd, 1H), 7.50 (d, 1H), 7.42 (m, 2H), 7.35 (s, 1H), 7.02 (d, 1H), 5.02 (s, 2H), 3.94 (m, 2H), 3.97 (m, 2H), 3.68 (m, 2H), 3.55 (m, 2H), 3.15 (m, 1H), 3.09 (m, 4H), 2.55 (m, 4H), 2.15 (s, 3H), 1.86 (m, 1H), 1.66 (m, 2H), 1.45 (m, 2H), 1.31 (m, 4H), 1.19 (m, 4H), 1.08 (m, 2H), 0.90 (s, 6H). MS (ESI) 912.2 (M+H)⁺.

### 1.16. Synthesis of 3-{1-[(3-{2-[L-alpha-aspartyl(methyl)amino]ethoxy}-5,7-dimethyltricyclo[3.3.1.1^{3,7}]dec-1-yl)methyl]-5-methyl-1H-pyrazol-4-yl}-6-[8-(1,3-benzothiazol-2-ylcarbamoyl)-3,4-dihydroisoquinolin-2(1H)-yl]pyridine-2-carboxylic Acid (Compound W2.17)

### 1.16.1. 6-[8-(1,3-benzothiazol-2-ylcarbamoyl)-3,4-dihydroisoquinolin-2(1H)-yl]-3-{1-[(3-{2-[{(2S)-4-tert-butoxy-2-[(tert-butoxycarbonyl)amino]-4-oxobutanoyl}(methyl)amino]ethoxy}-5,7-dimethyltricyclo[3.3.1.1^{3,7}]dec-1-yl)methyl]-5-methyl-1H-pyrazol-4-yl}pyridine-2-carboxylic Acid

A solution of Example 1.13.7 (0.060 g), (S)-4-tert-butyl 1-(2,5-dioxopyrrolidin-1-yl) 2-((tert-butoxycarbonyl)amino)succinate (0.034 g) and N,N-diisopropylethylamine were stirred together in dichloromethane (1 mL). After stirring overnight, the reaction was loaded onto silica gel and eluted using a gradient of 0.5-5% methanol/dichloromethane to give the title compound.

### 1.16.2. 3-{1-[(3-{2-[L-alpha-aspartyl(methyl)amino]ethoxy}-5,7-dimethyltricyclo[3.3.1.1^{3,7}]dec-1-yl)methyl]-5-methyl-1H-pyrazol-4-yl}-6-[8-(1,3-benzothiazol-2-ylcarbamoyl)-3,4-dihydroisoquinolin-2(1H)-yl]pyridine-2-carboxylic Acid

A solution of Example 1.17.1 (0.049 g) in dichloromethane (1 mL) was treated with trifluoroacetic acid (0.5 mL), and the reaction was stirred overnight. The reaction was concentrated, dissolved in N,N-dimethylformamide (2 mL) and water (0.5 mL) then purified by reverse phase HPLC using a Gilson system, eluting with 20-80% acetonitrile in water containing 0.1% v/v trifluoroacetic acid. The desired fractions were combined and freeze-dried to provide the title compound. ¹H NMR (400 MHz, dimethyl sulfoxide-d₆) δ ppm 12.85 (s, 1H), 8.15 (d, 3H), 8.03 (d, 1H), 7.79 (d, 1H), 7.62 (d, 1H), 7.55-7.41 (m, 3H), 7.36 (td, 2H), 7.29 (d, 1H), 6.95 (d, 1H), 4.96 (s, 2H), 4.55 (s, 1H), 3.92-3.86 (m, 2H), 3.60-3.47 (m, 2H), 3.47-3.37 (m, 2H), 3.32-3.21 (m, 1H), 3.09-2.97 (m, 4H), 2.92-2.72 (m, 3H), 2.67-2.53 (m, 1H), 2.10 (s, 3H), 1.46-0.94 (m, 12H), 0.85 (s, 6H). MS (ESI) m/e 875.2 (M+H)⁺.

### 1.17. Synthesis of 6-{4-[({2-[2-(2-aminoethoxy)ethoxy]ethyl}[2-({3-[(4-{6-[8-(1,3-benzothiazol-2-ylcarbamoyl)-3,4-dihydroisoquinolin-2(1H)-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl]amino)methyl]benzyl}-2,6-anhydro-L-gluconic Acid (Compound W2.18)

### 1.17.1. (2S,3S,4R,5S)-3,4,5-Triacetoxy-6-(4-bromomethyl-benzyl)-tetrahydro-pyran-2-carboxylic Acid Methyl Ester

The title compound was prepared as described in J. R. Walker et al., Bioorg. Med. Chem. 2006, 14, 3038-3048. MS (ESI) m/e 518, 520 (M+NH₄)⁺.

### 1.17.2. (2S,3S,4R,5S)-3,4,5-Triacetoxy-6-(4-formyl-benzyl)-tetrahydro-pyran-2-carboxylic Acid Methyl Ester

Example 1.18.1 (75 mg) and pyridine N-oxide (14 mg) were added to acetonitrile (0.75 mL). Silver (I) oxide (24 mg) was added to the solution, and the solution was stirred at room temperature for 16 hours. Anhydrous sodium sulfate (5 mg) was added, and the solution was stirred for five minutes. The solution was filtered and concentrated. The crude material was purified by flash column chromatography on silica gel, eluting with 50-70% ethyl acetate in heptanes. The solvent was evaporated under reduced pressure to provide the title compound.

### 1.17.3. (3R,4S,5R,6R)-2-(4-(((2-((3-((4-(6-(8-(benzo[d]thiazol-2-ylcarbamoyl)-3,4-dihydroisoquinolin-2(1H)-yl)-2-(tert-butoxycarbonyl)pyridin-3-yl)-5-methyl-1H-pyrazol-1-yl)methyl)-5,7-dimethyladamantan-1-yl)oxy)ethyl)amino)methyl)benzyl)-6-(methoxycarbonyl)tetrahydro-2H-pyran-3,4,5-triyl Triacetate

The title compound was prepared by substituting Example 1.18.2 for Example 1.5.3 in Example 1.5.4. MS (ESI) m/e 1222 (M+H)⁺.

### 1.17.4. {2-[2-(2-Oxo-ethoxy)-ethoxy]-ethyl}-carbamic Acid Tert-Butyl Ester

The title compound was prepared by substituting {2-[2-(2-hydroxy-ethoxy)-ethoxy]-ethyl}-carbamic acid tert-butyl ester for Example 1.5.1 in Example 1.5.2.

### 1.17.5. (3R,4S,5R,6R)-2-(4-(2-(2-((3-((4-(6-(8-(benzo[d]thiazol-2-ylcarbamoyl)-3,4-dihydroisoquinolin-2(1H)-yl)-2-(tert-butoxycarbonyl)pyridin-3-yl)-5-methyl-1H-pyrazol-1-yl)methyl)-5,7-dimethyladamantan-1-yl)oxy)ethyl)-14,14-dimethyl-12-oxo-5,8,13-trioxa-2,11-diazapentadecyl)benzyl)-6-(methoxycarbonyl)tetrahydro-2H-pyran-3,4,5-triyl Triacetate

The title compound was prepared by substituting Example 1.18.3 for Example 1.2.7 and Example 1.18.4 for Example 1.5.3 in Example 1.5.4. MS (ESI) m/e 1453 (M+H)⁺.

### 1.17.6. 6-{4-[({2-[2-(2-aminoethoxy)ethoxy]ethyl}[2-({3-[(4-{6-[8-(1,3-benzothiazol-2-ylcarbamoyl)-3,4-dihydroisoquinolin-2(1H)-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl]amino)methyl]benzyl}-2,6-anhydro-L-gluconic Acid

The title compound was prepared by substituting Example 1.18.5 for Example 1.5.4 in Example 1.5.5. ¹H NMR (400 MHz, dimethyl sulfoxide-d₆) δ ppm 9.38 (bs, 1H), 8.05 (dd, 1H), 7.90-7.68 (m, 6H), 7.62 (m, 2H), 7.53-7.27 (m, 8H), 6.94 (d, 1H), 4.96 (bs, 1H), 4.38 (bs, 4H), 3.91-3.57 (m, 11H), 3.37-3.11 (m, 14H), 2.98 (m, 6H), 2.61 (m, 1H), 2.10 (s, 3H), 1.44 (bs, 2H), 1.26 (m, 4H), 1.18-0.90 (m, 6H), 0.87 (bs, 6H). MS (ESI) m/e 1157 (M+H)⁺.

### 1.18. Synthesis of 4-({[2-({3-[(4-{6-[8-(1,3-benzothiazol-2-ylcarbamoyl)-3,4-dihydroisoquinolin-2(1H)-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl]amino}methyl)phenyl Hexopyranosiduronic Acid (Compound W2.19)

### 1.18.1. (2R,3S,4R,5R,6R)-2-(4-formylphenoxy)-6-(methoxycarbonyl)tetrahydro-2H-pyran-3,4,5-triyl Triacetate

To a solution of (2R,3R,4S,5S,6S)-2-bromo-6-(methoxycarbonyl)tetrahydro-2H-pyran-3,4,5-triyl triacetate (2.42 g) in acetonitrile (30 mL) was added silver(I) oxide (1.4 g) and 4-hydroxybenzaldehyde (620 mg). The reaction mixture was stirred for 4 hours and filtered. The filtrate was concentrated, and the residue was purified by silica gel chromatography, eluting with 5-50% ethyl acetate in heptanes, to provide the title compound. MS (ESI) m/e 439.2 (M+H)⁺.

### 1.18.2. 4-({[2-({3-[(4-{6-[8-(1,3-benzothiazol-2-ylcarbamoyl)-3,4-dihydroisoquinolin-2(1H)-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl]amino}methyl)phenyl Hexopyranosiduronic Acid

To a solution of Example 1.2.7 (36 mg) in tetrahydrofuran (2 mL) and acetic acid (0.2 mL) was added Example 1.19.1 (21 mg) followed by MgSO₄ (60 mg). The mixture was stirred for 1 hour before the addition of NaBH₃CN on resin (153 mg). The mixture was then stirred for 3 hours. The mixture was filtered and lithium hydroxide monohydrate (20 mg) was added to the filtrate. The mixture was stirred for 2 hours and was acidified with trifluoroacetic acid and purified by reverse phase HPLC (Gilson system), eluting with 10-85% acetonitrile in 0.1% trifluoroacetic acid in water, to give the title compound. ¹H NMR (400 MHz, dimethyl sulfoxide-d₆) δ ppm 12.86 (s, 1H), 8.57-8.72 (m, 2H), 8.03 (d, 1H), 7.79 (d, 1H), 7.62 (d, 1H), 7.34-7.53 (m, 6H), 7.08 (t, 2H), 6.95 (d, 1H), 5.10 (d, 1H), 4.96 (s, 2H), 4.06-4.15 (m, 4H), 3.83-3.97 (m, 6H), 3.26-3.42 (m, 8H), 2.93-3.10 (m, 6H), 2.10 (s, 3H), 1.43 (s, 2H), 1.24-1.38 (m, 6H), 0.97-1.16 (m, 4H), 0.86 (s, 6H). MS (ESI) m/e 1028.3 (M+H)⁺.

### 1.19. Synthesis of 6-[1-(1,3-benzothiazol-2-ylcarbamoyl)-1,2,3,4-tetrahydroquinolin-7-yl]-3-{1-[(3,5-dimethyl-7-{2-[(2-phosphonoethyl)amino]ethoxy}tricyclo[3.3.1.1^{3,7}]dec-1-yl)methyl]-5-methyl-1H-pyrazol-4-yl}pyridine-2-carboxylic Acid (Compound W2.20)

### 1.19.1. 2-((3,5-dimethyl-7-((5-methyl-4-(4,4,5,5-tetramethyl-1,3,2-dioxaborolan-2-yl)-1H-pyrazol-1-yl)methyl)adamantan-1-yl)oxy)ethanol

To a solution of Example 1.1.6 (9 g) and [1,1′-bis(diphenylphosphino)ferrocene]dichloropalladium(II) dichloromethane (827 mg) in acetonitrile (60 mL) was added triethylamine (10 mL) and pinacolborane (6 mL). The mixture was stirred at reflux overnight, cooled and used directly in the next step. MS (ESI) m/e 445.4 (M+H)⁺.

### 1.19.2. tert-butyl 6-chloro-3-(1-((3-(2-hydroxyethoxy)-5,7-dimethyladamantan-1-yl)methyl)-5-methyl-1H-pyrazol-4-yl)picolinate

To a solution of tert-butyl 3-bromo-6-chloropicolinate (5.92 g) in tetrahydrofuran (60 mL) and water (30 mL) was added the crude Example 1.20.1 (4.44 g), 1,3,5,7-tetramethyl-6-phenyl-2,4,8-trioxa-6-phosphaadamantane (1.5 g), tris(dibenzylideneacetone)dipalladium(0) (927 mg) and K₃PO₄ (22 g). The mixture was stirred at reflux overnight, cooled, diluted with ethyl acetate (800 mL) and washed with water and brine. The organic layer was dried over sodium sulfate, filtered, and concentrated. The residue was purified by flash chromatography, eluting with 20% ethyl acetate in heptane followed by 5% methanol in dichloromethane, to give the title compound. MS (ESI) m/e 531.1 (M+H)⁺.

### 1.19.3. tert-butyl 3-(1-((3-(2-((tert-butyldimethylsilyl)oxy)ethoxy)-5,7-dimethyladamantan-1-yl)methyl)-5-methyl-1H-pyrazol-4-yl)-6-chloropicolinate

To a solution of Example 1.20.2 (3.2 g) in N,N-dimethylformamide (20 mL) was added imidazole (0.62 g) and chloro t-butyldimethylsilane (1.37 g). The mixture was stirred overnight, diluted with ethyl acetate (300 mL), and washed with water and brine. The organic layer was dried over sodium sulfate, filtered, and concentrated. The residue was purified by flash chromatography, eluting with 20% ethyl acetate in heptanes, to provide the title compound. MS (ESI) m/e 645.4 (M+H)⁺.

### 1.19.4. tert-butyl 3-(1-((3-(2-((tert-butyldimethylsilyl)oxy)ethoxy)-5,7-dimethyladamantan-1-yl)methyl)-5-methyl-1H-pyrazol-4-yl)-6-(1,2,3,4-tetrahydroquinolin-7-yl)picolinate

To a solution of 7-(4,4,5,5-tetramethyl-1,3,2-dioxaborolan-2-yl)-1,2,3,4-tetrahydroquinoline (507 mg) in 1,4-dioxane (10 mL) and water (5 mL) was added Example 1.20.3 (1.25 g), bis(triphenylphosphine)palladium(II)dichloride (136 mg), and cesium fluoride (884 mg). The mixture was heated at 120° C. in a microwave synthesizer (Biotage, Initiator) for 20 minutes. The mixture was diluted with ethyl acetate (500 mL), and washed with water and brine. The organic layer was dried over sodium sulfate, filtered, concentrated and purified by flash chromatography, eluting with 20% ethyl acetate in heptanes and then with 5% methanol in dichloromethane, to provide the title compound. MS (ESI) m/e 741.5 (M+H)⁺.

### 1.19.5. tert-butyl 6-(1-(benzo[d]thiazol-2-ylcarbamoyl)-1,2,3,4-tetrahydroquinolin-7-yl)-3-(1-(3-(2-((tert-butyldimethylsilyl)oxy)ethoxy)-5,7-dimethyladamantan-1-yl)methyl)-5-methyl-1H-pyrazol-4-yl)picolinate

To a suspension of bis(2,5-dioxopyrrolidin-1-yl) carbonate (295 mg) in acetonitrile (10 mL) was added benzo[d]thiazol-2-amine (173 mg), and the mixture was stirred for 1 hour. A solution of Example 1.20.4 (710 mg) in acetonitrile (10 mL) was added, and the suspension was stirred overnight. The mixture was diluted with ethyl acetate (300 mL), washed with water and brine and dried over sodium sulfate. After filtration, the organic layer was concentrated and purified by silica gel chromatography, eluting with 20% ethyl acetate in heptanes, to provide the title compound. MS (ESI) m/e 917.2 (M+H)⁺.

### 1.19.6. tert-butyl 6-(1-(benzo[d]thiazol-2-ylcarbamoyl)-1,2,3,4-tetrahydroquinolin-7-yl)-3-(1-((3-(2-hydroxyethoxy)-5,7-dimethyladamantan-1-yl)methyl)-5-methyl-1H-pyrazol-4-yl)picolinate

To a solution of Example 1.20.5 (1.4 g) in tetrahydrofuran (10 mL) was added tetrabutyl ammonium fluoride (1.0 M in tetrahydrofuran, 6 mL). The mixture was stirred for 3 hours, diluted with ethyl acetate (300 mL) and washed with water and brine. The organic layer was dried over sodium sulfate, filtered, and concentrated to provide the title compound. MS (ESI) m/e 803.4 (M+H)⁺.

### 1.19.7. tert-butyl 6-(1-(benzo[d]thiazol-2-ylcarbamoyl)-1,2,3,4-tetrahydroquinolin-7-yl)-3-(1-((3,5-dimethyl-7-(2-((methylsulfonyl)oxy)ethoxy)adamantan-1-yl)methyl)-5-methyl-1H-pyrazol-4-yl)picolinate

To a cooled (0° C.) solution of Example 1.20.6 (1.2 g) in dichloromethane (20 mL) and triethylamine (2 mL) was added methanesulfonyl chloride (300 mg). The mixture was stirred for 4 hours, diluted with ethyl acetate (200 mL) and washed with water and brine. The organic layer was dried over sodium sulfate, filtered, and concentrated to provide the title compound. MS (ESI) m/e 881.3 (M+H)⁺.

### 1.19.8. tert-butyl 3-(1-((3-(2-azidoethoxy)-5,7-dimethyladamantan-1-yl)methyl)-5-methyl-1H-pyrazol-4-yl)-6-(1-(benzo[d]thiazol-2-ylcarbamoyl)-1,2,3,4-tetrahydroquinolin-7-yl)picolinate

To a solution of Example 1.20.7 (1.5 g) in N,N-dimethylformamide (20 mL) was added sodium azide (331 mg). The mixture was stirred for 48 hours, diluted with ethyl acetate (20.0 mL) and washed with water and brine. The organic layer was dried over sodium sulfate, filtered, concentrated and purified by silica gel chromatography, eluting with 20% ethyl acetate in dichloromethane, to provide the title compound. MS (ESI) m/e 828.4 (M+H)⁺.

### 1.19.9. tert-butyl 3-(1-((3-(2-aminoethoxy)-5,7-dimethyladamantan-1-yl)methyl)-5-methyl-1H-pyrazol-4-yl)-6-(1-(benzo[d]thiazol-2-ylcarbamoyl)-1,2,3,4-tetrahydroquinolin-7-yl)picolinate

To a solution of Example 1.20.8 (1.5 g) in tetrahydrofuran (30 mL) was added Pd/C (10%, 200 mg). The mixture was stirred under a hydrogen atmosphere overnight. The reaction was filtered, and the filtrate was concentrated to provide the title compound. MS (ESI) m/e 802.4 (M+H)⁺.

### 1.19.10. tert-butyl 6-(1-(benzo[d]thiazol-2-ylcarbamoyl)-1,2,3,4-tetrahydroquinolin-7-yl)-3-(1-((3-(2-((2-(diethoxyphosphoryl)ethyl)amino)ethoxy)-5,7-dimethyladamantan-1-yl)methyl)-5-methyl-1H-pyrazol-4-yl)picolinate

The title compound was prepared as described in Example 1.12.1, replacing Example 1.2.7 with Example 1.20.9.

### 1.19.11.6-[1-(1,3-benzothiazol-2-ylcarbamoyl)-1,2,3,4-tetrahydroquinolin-7-yl]-3-{1-[(3,5-dimethyl-7-{2-[(2-phosphonoethyl)amino]ethoxy}tricyclo[3.3.1.1^{3,7}]dec-1-yl)methyl]-5-methyl-1H-pyrazol-4-yl}pyridine-2-carboxylic Acid

The title compound was prepared as described in Example 1.12.2, replacing Example 1.12.1 with Example 1.20.10. ¹H NMR (500 MHz, dimethyl sulfoxide-d₆) δ ppm 8.40 (s, 2H), 8.02 (d, 1H), 7.74-7.89 (m, 3H), 7.47 (s, 2H), 7.38 (t, 1H), 7.30 (d, 1H), 7.23 (t, 1H), 3.96 (s, 2H), 3.90 (s, 2H), 3.53-3.64 (m, 2H), 3.03-3.18 (m, 2H), 2.84 (t, 2H), 2.23 (s, 3H), 1.87-2.02 (m, 4H), 1.46 (s, 2H), 1.26-1.38 (m, 4H), 1.12-1.23 (m, 4H), 0.99-1.11 (m, 2H), 0.89 (s, 6H). MS (ESI) m/e 854.1 (M+H)⁺.

### 1.20. Synthesis of 6-[1-(1,3-benzothiazol-2-ylcarbamoyl)-1,2,3,4-tetrahydroquinolin-7-yl]-3-{1-[(3,5-dimethyl-7-{2-[methyl(3-sulfo-L-alanyl)amino]ethoxy}tricyclo[3.3.1.1^{3,7}]dec-1-yl)methyl]-5-methyl-1H-pyrazol-4-yl}pyridine-2-carboxylic Acid (Compound W2.21)

### 1.20.1. tert-butyl (2-((3,5-dimethyl-7-((5-methyl-4-(4,4,5,5-tetramethyl-1,3,2-dioxaborolan-2-yl)-1H-pyrazol-1-yl)methyl)adamantan-1-yl)oxy)ethyl)(methyl)carbamate

To a solution of Example 1.13.3 (1.2 g) in 1,4-dioxane was added bis(benzonitrile)palladium(II) chloride (0.04 g), 4,4,5,5-tetramethyl-1,3,2-dioxaborolane (0.937 mL) and triethylamine (0.9 mL). The mixture was heated at reflux overnight, diluted with ethyl acetate and washed with water (60 mL) and brine (60 mL). The organic layer was dried over sodium sulfate, filtered and concentrated to provide the title compound.

### 1.20.2. tert-butyl 3-(1-((3-(2-((tert-butoxycarbonyl)(methyl)amino)ethoxy)-5,7-dimethyladamantan-1-yl)methyl)-5-methyl-1H-pyrazol-4-yl)-6-chloropicolinate

The title compound was prepared as described in Example 1.1.12, replacing Example 1.1.11 and Example 1.1.8 with tert-butyl 3-bromo-6-chloropicolinate and Example 1.21.1, respectively. MS (APCI) m/e 643.9 (M+H)⁺.

### 1.20.3. tert-butyl 3-(1-((3-(2-((tert-butoxycarbonyl)(methyl)amino)ethoxy)-5,7-dimethyladamantan-1-yl)methyl)-5-methyl-1H-pyrazol-4-yl)-6-(1,2,3,4-tetrahydroquinolin-7-yl)picolinate

A mixture of Example 1.21.2 (480 mg), 7-(4,4,5,5-tetramethyl-1,3,2-dioxaborolan-2-yl)-1,2,3,4-tetrahydroquinoline (387 mg), dichlorobis(triphenylphosphine)-palladium(II) (78 mg) and cesium fluoride (340 mg) in 1,4-dioxane (12 mL) and water (5 mL) was heated at 100° C. for 5 hours. The reaction was cooled and diluted with ethyl acetate. The resulting mixture was washed with water and brine, and the organic layer was dried over sodium sulfate, filtered, and concentrated. The residue was purified by flash chromatography, eluting with 50% ethyl acetate in heptanes, to provide the title compound. MS (APCI) m/e 740.4 (M+H)⁺.

### 1.20.4. tert-butyl 6-(1-(benzo[d]thiazol-2-ylcarbamoyl)-1,2,3,4-tetrahydroquinolin-7-yl)-3-(1-((3-(2-((tert-butoxycarbonyl)(methyl)amino)ethoxy)-5,7-dimethyladamantan-1-yl)methyl)-5-methyl-1H-pyrazol-4-yl)picolinate

To a solution of benzo[d]thiazol-2-amine (114 mg) in acetonitrile (5 mL) was added bis(2,5-dioxopyrrolidin-1-yl) carbonate (194 mg). The mixture was stirred for 1 hour, and Example 1.21.3 (432 mg) in acetonitrile (5 mL) was added. The mixture was stirred overnight, diluted with ethyl acetate, washed with water and brine. The organic layer was dried over sodium sulfate, filtered, and concentrated. The residue was purified by silica gel chromatography, eluting with 50% ethyl acetate in heptanes, to provide the title compound.

### 1.20.5. 6-(1-(benzo[d]thiazol-2-ylcarbamoyl)-1,2,3,4-tetrahydroquinolin-7-yl)-3-(1-((3,5-dimethyl-7-(2-(methylamino)ethoxy)adamantan-1-yl)methyl)-5-methyl-1H-pyrazol-4-yl)picolinic Acid

Example 1.2.4 (200 mg) in dichloromethane (5 mL) was treated with trifluoroacetic acid (2.5 mL) overnight. The mixture was concentrated to provide the title compound. ¹H NMR (400 MHz, dimethyl sulfoxide-d₆) δ ppm 8.40 (s, 1H), 8.30 (s, 2H), 8.02 (d, 1H), 7.85 (d, 1H), 7.74-7.83 (m, 2H), 7.42-7.53 (m, 2H), 7.38 (t, 1H), 7.30 (d, 1H), 7.23 (t, 1H), 3.93-4.05 (m, 2H), 3.52-3.62 (m, 2H), 2.97-3.10 (m, 2H), 2.84 (t, 2H), 2.56 (t, 2H), 2.23 (s, 3H), 1.88-2.00 (m, 2H), 1.45 (s, 2H), 1.25-1.39 (m, 4H), 1.12-1.22 (m, 4H), 1.00-1.09 (m, 2H), 0.89 (s, 6H). MS (ESI) m/e 760.1 (M+H)⁺.

### 1.20.6. 6-(1-(benzo[d]thiazol-2-ylcarbamoyl)-1,2,3,4-tetrahydroquinolin-7-yl)-3-(1-((3-(2-((R)-2-((tert-butoxycarbonyl)amino)-N-methyl-3-sulfopropanamido)ethoxy)-5,7-dimethyladamantan-1-yl)methyl)-5-methyl-1H-pyrazol-4-yl)picolinic Acid

(R)-2-((tert-butoxycarbonyl)amino)-3-sulfopropanoic acid (70.9 mg) and O-(7-azabenzotriazol-1-yl)-N,N,N′,N′-tetramethyluroniumhexafluorophosphate (HATU, 65 mg) in N,N-dimethylformamide (1.5 ml) was cooled in ice-bath, and N,N-diisopropylethylamine (68.9 μL) was added. The mixture was stirred at 0° C. for 15 minutes and at room temperature for 8 hours. Example 1.21.5 (100 mg) in N,N-dimethylformamide (1 mL) and N,N-diisopropylethylamine (60 μL) were added. The resulting mixture was stirred overnight, concentrated and purified by reverse phase chromatography (C18 column), eluting with 20-60% acetonitrile in water containing 0.1% trifluoroacetic acid, to provide the title compound.

### 1.20.7. 6-[1-(1,3-benzothiazol-2-ylcarbamoyl)-1,2,3,4-tetrahydroquinolin-7-yl]-3-{1-[(3,5-dimethyl-7-{2-[methyl(3-sulfo-L-alanyl)amino]ethoxy}tricyclo[3.3.1.1^{3,7}]dec-1-yl)methyl]-5-methyl-1H-pyrazol-4-yl}pyridine-2-carboxylic Acid

Example 1.21.6 (80 mg) in dichloromethane (3 mL) was treated with trifluoroacetic acid (1.5 mL) for 20 minutes. The reaction mixture was concentrated and purified by reverse phase chromatography (C18 column), eluting with 0-50% acetonitrile in 4 mM aqueous ammonium acetate solution, to provide the title compound. ¹H NMR (500 MHz, dimethyl sulfoxide-d₆) δ ppm 8.57 (s, 1H), 7.59-7.67 (m, 3H), 7.54 (d, 1H), 7.46-7.51 (m, 1H), 7.30 (d, 1H), 7.08-7.17 (m, 2H), 6.90 (t, 1H), 3.91-4.10 (m, 3H), 3.84 (s, 2H), 3.04 (s, 2H), 2.75-2.83 (m, 4H), 2.59-2.70 (m, 2H), 2.27-2.39 (m, 2H), 2.26 (s, 3H), 1.81-1.93 (m, 2H), 1.74 (s, 9H), 1.42 (s, 2H), 0.96-1.33 (m, 10H), 0.86 (s, 3H). MS (ESI) m/e 909.2 (M−H)⁻.

### 1.21. Synthesis of 3-{1-[(3,5-dimethyl-7-{2-[(2-sulfoethyl)amino]ethoxy}tricyclo[3.3.1.1^{3,7}]dec-1-yl)methyl]-5-methyl-1H-pyrazol-4-yl}-6-[8-([1,3]thiazolo[5,4-b]pyridin-2-ylcarbamoyl)-3,4-dihydroisoquinolin-2(1H)-yl]pyridine-2-carboxylic Acid (Compound W2.22)

### 1.21.1. tert-butyl 3-(1-((3-(2-azidoethoxy)-5,7-dimethyladamantan-1-yl)methyl)-5-methyl-1H-pyrazol-4-yl)-6-(8-(thiazolo[5,4-b]pyridin-2-ylcarbamoyl)-3,4-dihydroisoquinolin-2(1H)-yl)picolinate

Example 1.2.5 (560 mg) and thiazolo[5,4-b]pyridin-2-amine (135 mg) were dissolved in dichloromethane (12 mL). N,N-Dimethylpyridin-4-amine (165 mg) and N-ethyl-N′-(3-dimethylaminopropyl)carbodiimide hydrochloride (260 mg) were added, and the reaction stirred at room temperature overnight. The reaction mixture was concentrated, and the crude residue was purified by silica gel chromatography, eluting with 65/35 dichloromethane/ethyl acetate, to provide the title compound. MS (ESI) m/e 829.1 (M+H)⁺.

### 1.21.2. tert-butyl 3-(1-((3-(2-aminoethoxy)-5,7-dimethyladamantan-1-yl)methyl)-5-methyl-1H-pyrazol-4-yl)-6-(S-(thiazolo[5,4-b]pyridin-2-ylcarbamoyl)-3,4-dihydroisoquinolin-2(1H)-yl)picolinate

The title compound was prepared by substituting Example 1.22.1 for Example 1.2.6 in Example 1.2.7. MS (ESI) m/e 803.2 (M+H)⁺.

### 1.21.3. tert-butyl 3-[1-({3,5-dimethyl-7-[(2,2,7,7-tetramethyl-10,10-dioxido-3,3-diphenyl-4,9-dioxa-10λ⁶-thia-13-aza-3-silapentadecan-15-yl)oxy]tricyclo[3.3.1.1^{3,7}]dec-1-yl}methyl)-5-methyl-1H-pyrazol-4-yl]-6-[S-([1,3]thiazolo[5,4-b]pyridin-2-ylcarbamoyl)-3,4-dihydroisoquinolin-2(1H)-yl]pyridine-2-carboxylate

To a solution of Example 1.22.2 (70 mg) and 4-((tert-butyldiphenylsilyl)oxy)-2,2-dimethylbutyl ethanesulfonate (48 mg) in dichloromethane (1 mL) was added N,N-diisopropylethylamine (0.06 mL), and the reaction stirred at room temperature overnight. The reaction was concentrated, and the crude residue was purified by silica gel chromatography, eluting with a gradient of 1-4% methanol in dichloromethane, to provide the title compound. MS (ESI) m/e 1249.2 (M+H)⁺.

### 1.21.4. 2-((2-((3-((4-(2-(tert-butoxycarbonyl)-6-(8-(thiazolo[5,4-b]pyridin-2-ylcarbamoyl)-3,4-dihydroisoquinolin-2(1H)-yl)pyridin-3-yl)-5-methyl-1H-pyrazol-1-yl)methyl)-5,7-dimethyladamantan-1-yl)oxy)ethyl)amino)ethanesulfonic Acid

To a solution of Example 1.22.3 (70 mg) in tetrahydrofuran (0.25 mL) was added tetrabutylammonium fluoride (60 μL, 1.0M solution in tetrahydrofuran), and the reaction was stirred at room temperature for two days. The reaction was concentrated, and the residue was purified by reverse phase chromatography (C18 column), eluting with 10-90% acetonitrile in water containing 0.1% trifluoroacetic acid, to provide the title compound as a trifluoroacetic acid salt. MS (ESI) m/e 911.1 (M+H)⁺.

### 1.21.5. 3-{1-[(3,5-dimethyl-7-{2-[(2-sulfoethyl)amino]ethoxy}tricyclo[3.3.1.1^{3,7}]dec-1-yl)methyl]-5-methyl-1H-pyrazol-4-yl}-6-[8-([1,3]thiazolo[5,4-b]pyridin-2-ylcarbamoyl)-3,4-dihydroisoquinolin-2(1H)-yl]pyridine-2-carboxylic Acid

The title compound was prepared by substituting Example 1.22.4 for Example 1.2.8 in Example 1.2.9. ¹H NMR (400 MHz, dimethyl sulfoxide-d₆) δ ppm 13.00 (s, 1H), 8.52 (dd, 2H), 8.33 (br s, 2H), 8.16 (dd, 1H), 7.62 (m, 1H), 7.53 (m, 2H), 7.45 (d, 1H), 7.38 (m, 1H), 7.29 (s, 1H), 6.98 (d, 1H), 4.96 (s, 2H), 3.88 (m, 2H), 3.83 (s, 2H), 3.54 (m, 2H), 3.22 (m, 2H), 3.10 (m, 2H), 3.02 (t, 2H), 2.80 (t, 2H), 2.11 (s, 3H), 1.41 (s, 2H), 1.28 (m, 4H), 1.14 (m, 4H), 1.02 (m, 2H), 0.86 (s, 6H). MS (ESI) m/e 855.2 (M+H)⁺.

### 1.22. Synthesis of 3-{1-[(3,5-dimethyl-7-{2-[(2-sulfoethyl)amino]ethoxy}tricyclo[3.3.1.1^{3,7}]dec-1-yl)methyl]-5-methyl-1H-pyrazol-4-yl}-6-[8-([1,3]thiazolo[4,5-b]pyridin-2-ylcarbamoyl)-3,4-dihydroisoquinolin-2(1H)-yl]pyridine-2-carboxylic Acid (Compound W2.23)

### 1.22.1. tert-butyl 3-(1-((3-(2-azidoethoxy)-5,7-dimethyladamantan-1-yl)methyl)-5-methyl-1H-pyrazol-4-yl)-6-(8-(thiazolo[4,5-b]pyridin-2-ylcarbamoyl)-3,4-dihydroisoquinolin-2(1H)-yl)picolinate

The title compound was prepared by substituting thiazolo[4,5-b]pyridin-2-amine for thiazolo[5,4-b]pyridin-2-amine in Example 1.22.1. MS (ESI) m/e 855.2 (M+H)⁺.

### 1.22.2. tert-butyl 3-(1-((3-(2-aminoethoxy)-5,7-dimethyladamantan-1-yl)methyl)-5-methyl-1H-pyrazol-4-yl)-6-(8-(thiazolo[4,5-b]pyridin-2-ylcarbamoyl)-3,4-dihydroisoquinolin-2(1H)-yl)picolinate

The title compound was prepared by substituting Example 1.23.1 for Example 1.2.6 in Example 1.2.7. MS (ESI) m/e 803.2 (M+H)⁺.

### 1.22.3. tert-butyl 3-[1-({3,5-dimethyl-7-[(2,2,7,7-tetramethyl-10,10-dioxido-3,3-diphenyl-4,9-dioxa-10λ⁶-thia-13-aza-3-silapentadecan-15-yl)oxy]tricyclo[3.3.1.1^{3,7}]dec-1-yl}methyl)-5-methyl-1H-pyrazol-4-yl]-6-[8-([1,3]thiazolo[4,5-b]pyridin-2-ylcarbamoyl)-3,4-dihydroisoquinolin-2(1H)-yl]pyridine-2-carboxylate

The title compound was prepared by substituting Example 1.23.2 for Example 1.22.2 in Example 1.22.3. MS (ESI) m/e 1249.2 (M+H)⁺.

### 1.22.4. 3-{1-[(3,5-dimethyl-7-{2-[(2-sulfoethyl)amino]ethoxy}tricyclo[3.3.1.1^{3,7}]dec-1-yl)methyl]-5-methyl-1H-pyrazol-4-yl}-6-[8-([1,3]thiazolo[4,5-b]pyridin-2-ylcarbamoyl)-3,4-dihydroisoquinolin-2(1H)-yl]pyridine-2-carboxylic Acid

The title compound was prepared by substituting Example 1.23.3 for Example 1.2.8 in Example 1.2.9. ¹H NMR (400 MHz, dimethyl sulfoxide-d₆) δ ppm 13.20 (br s, 1H), 8.61 (dd, 1H), 8.56 (dd, 1H), 8.33 (br s, 2H), 7.56 (d, 1H) 7.52 (d, 1H), 7.46 (d, 1H), 7.39 (m, 2H), 7.29 (s, 1H), 6.98 (d, 1H), 4.98 (s, 2H), 3.88 (m, 2H), 3.83 (s, 2H), 3.54 (m, 2H), 3.22 (m, 2H), 3.10 (m, 2H), 3.02 (t, 2H), 2.80 (t, 2H), 2.10 (s, 3H), 1.41 (s, 2H), 1.30 (m, 4H), 1.12 (m, 4H), 1.02 (m, 2H), 0.86 (s, 6H). MS (ESI) m/e 855.1 (M+H)⁺.

### 1.23. Synthesis of 6-[1-(1,3-benzothiazol-2-ylcarbamoyl)-1,2,3,4-tetrahydroquinolin-7-yl]-3-{1-[(3,5-dimethyl-7-{2-[(2-sulfoethyl)amino]ethoxy}tricyclo[3.3.1.1^{3,7}]dec-1-yl)methyl]-5-methyl-1H-pyrazol-4-yl}pyridine-2-carboxylic Acid (Compound W2.24)

### 1.23.1. The Title Compound was Prepared as Described in Example 1.2.8, Replacing Example 1.2.7 with Example 1.20.9

### 1.23.2. 6-[1-(1,3-benzothiazol-2-ylcarbamoyl)-1,2,3,4-tetrahydroquinolin-7-yl]-3-{1-[(3,5-dimethyl-7-{2-[(2-sulfoethyl)amino]ethoxy}tricyclo[3.3.1.1^{3,7}]dec-1-yl)methyl]-5-methyl-1H-pyrazol-4-yl}pyridine-2-carboxylic Acid

The title compound was prepared as described in Example 1.2.9, replacing Example 1.2.8 with Example 1.24.1. ¹H NMR (500 MHz, dimethyl sulfoxide-d₆) δ ppm 8.26-8.46 (m, 3H), 8.02 (d, 1H), 7.89 (d, 1H), 7.82 (d, 1H), 7.75-7.79 (m, 1H), 7.47 (s, 2H), 7.37 (t, 1H), 7.30 (d, 1H), 7.22 (t, 1H), 3.96 (s, 2H), 3.90 (s, 2H), 3.54-3.61 (m, 2H), 3.18-3.29 (m, 2H), 3.07-3.15 (m, 2H), 2.78-2.92 (m, 4H), 2.23 (s, 3H), 1.87-2.02 (m, 2H), 1.44 (s, 2H), 1.32 (q, 4H), 1.12-1.25 (m, 4H), 1.00-1.11 (m, 2H), 0.88 (s, 6H). MS (ESI) m/e 854.0 (M+H)⁺.

### 1.24. 6-[8-(1,3-benzothiazol-2-ylcarbamoyl)-3,4-dihydroisoquinolin-2(1H)-yl]-3-{1-[(3-{2-[(2-carboxyethyl)amino]ethoxy}-5,7-dimethyltricyclo[3.3.1.1^{3,7}]dec-1-yl)methyl]-5-methyl-1H-pyrazol-4-yl}pyridine-2-carboxylic Acid

### 1.24.1. tert-butyl 6-(8-(benzo[d]thiazol-2-ylcarbamoyl)-3,4-dihydroisoquinolin-2(1H)-yl)-3-(1-((3-(2-((3-(tert-butoxy)-3-oxopropyl)amino)ethoxy)-5,7-dimethyladamantan-1-yl)methyl)-5-methyl-1H-pyrazol-4-yl)picolinate

The title compound was prepared as described in Example 1.12.1, replacing diethyl vinylphosphonate with tert-butyl acrylate. MS (APCI) m/e 930.6 (M+H)⁺.

### 1.24.2. 66-[8-(1,3-benzothiazol-2-ylcarbamoyl)-3,4-dihydroisoquinolin-2(1H)-yl]-3-{1-[(3-{2-[(2-carboxyethyl)amino]ethoxy}-5,7-dimethyltricyclo[3.3.1.1^{3,7}]dec-1-yl)methyl]-5-methyl-1H-pyrazol-4-yl}pyridine-2-carboxylic Acid

The title compound was prepared as described in Example 1.6.2, replacing Example 1.6.1 with Example 1.25.1. ¹H NMR (400 MHz, dimethyl sulfoxide-d₆) δ ppm 8.03 (d, 1H), 7.78 (d, 1H), 7.61 (d, 1H), 7.39-7.50 (m, 2H), 7.32-7.38 (m, 3H), 7.23 (s, 1H), 6.73 (d, 1H), 4.88 (s, 2H), 3.88 (t, 2H), 3.79 (s, 2H), 2.99 (t, 2H), 2.86-2.93 (m, 2H), 2.50-2.58 (m, 2H), 2.08 (s, 3H), 1.35 (d, 2H), 1.01-1.30 (m, 10H), 0.86 (s, 6H). MS (APCI) m/e 819.0 (M+H)⁺.

### 1.25. Synthesis of 6-[8-(1,3-benzothiazol-2-ylcarbamoyl)-3,4-dihydroisoquinolin-2(1H)-yl]-3-{1-[(3,5-dimethyl-7-{2-[(3-phosphonopropyl)(piperidin-4-yl)amino]ethoxy}tricyclo[3.3.1.1^{3,7}]dec-1-yl)methyl]-5-methyl-1H-pyrazol-4-yl}pyridine-2-carboxylic Acid (Compound W2.26)

### 1.25.1. tert-butyl 6-(8-(benzo[d]thiazol-2-ylcarbamoyl)-3,4-dihydroisoquinolin-2(1H)-yl)-3-(1-(((1r,3r)-3-(2-((1-(tert-butoxycarbonyl)piperidin-4-yl)amino)ethoxy)-5,7-dimethyladamantan-1-yl)methyl)-5-methyl-1H-pyrazol-4-yl)picolinate

A solution of Example 1.2.7 (0.020 g), tert-butyl 4-oxopiperidine-1-carboxylate (4.79 mg) and sodium triacetoxyborohydride (7 mg) was stirred in dichloromethane (0.5 mL) at room temperature. The reaction was stirred overnight and purified without workup by silica gel chromatography, eluting with 0 to 10% methanol in dichloromethane, to give the title compound. MS (ELSD) m/e 985.4 (M+H)⁺.

### 1.25.2. 6-[8-(1,3-benzothiazol-2-ylcarbamoyl)-3,4-dihydroisoquinolin-2(1H)-yl]-3-{1-[(3,5-dimethyl-7-{2-[(3-phosphonopropyl)(piperidin-4-yl)amino]ethoxy}tricyclo[3.3.1.1^{3,7}]dec-1-yl)methyl]-5-methyl-1H-pyrazol-4-yl}pyridine-2-carboxylic Acid

A solution of Example 1.26.1 (0.108 g), Example 1.14.2 (0.030 g) and sodium triacetoxyborohydride (0.035 g) in dichloromethane (1 mL) was stirred at room temperature for 1 hour. Trifluoroacetic acid (1 mL) was added to the reaction, and stirring was continued overnight. The reaction was concentrated, dissolved in N,N-dimethylformamide (2 mL) and water (0.5 mL) and purified by reverse phase HPLC using a Gilson system, eluting with 10-75% acetonitrile in water containing 0.1% v/v trifluoroacetic acid. The desired fractions were combined and freeze-dried to provide the title compound. ¹H NMR (400 MHz, dimethyl sulfoxide-d₆) δ ppm 8.83 (s, 1H), 8.50 (s, 1H), 8.04 (d, 2H), 7.80 (d, 2H), 7.63 (d, 2H), 7.56-7.42 (m, 5H), 7.37 (tt, 3H), 7.30 (s, 1H), 6.96 (d, 1H), 4.96 (s, 2H), 3.89 (t, 2H), 3.44 (d, 6H), 3.31-3.16 (m, 6H), 3.09-2.98 (m, 2H), 2.98-2.85 (m, 1H), 2.18 (d, 2H), 2.10 (s, 3H), 2.00-1.74 (m, 4H), 1.71-1.57 (m, 2H), 1.51-0.97 (m, 12H), 0.87 (s, 6H). MS (ESI) m/e 951.2 (M+H)⁺.

### 1.26. Synthesis of 3-{1-[(3-{2-[D-alpha-aspartyl(methyl)amino]ethoxy}-5,7-dimethyltricyclo[3.3.1.1^{3,7}]dec-1-yl)methyl]-5-methyl-1H-pyrazol-4-yl}-6-[8-(1,3-benzothiazol-2-ylcarbamoyl)-3,4-dihydroisoquinolin-2(1H)-yl]pyridine-2-carboxylic Acid (Compound W2.27)

### 1.26.1. tert-butyl 6-(8-(benzo[d]thiazol-2-ylcarbamoyl)-3,4-dihydroisoquinolin-2(1H)-yl)-3-(1-((3,5-dimethyl-7-(2-(methylamino)ethoxy)adamantan-1-yl)methyl)-5-methyl-1H-pyrazol-4-yl)picolinate

The title compound was prepared as described in Example 1.11.1 by substituting Example 1.10.9 with Example 1.13.6.

### 1.26.2. 3-{1-[(3-{2-[D-alpha-aspartyl(methyl)amino]ethoxy}-5,7-dimethyltricyclo[3.3.1.1^{3,7}]dec-1-yl)methyl]-5-methyl-1H-pyrazol-4-yl}-6-[8-(1,3-benzothiazol-2-ylcarbamoyl)-3,4-dihydroisoquinolin-2(1H)-yl]pyridine-2-carboxylic Acid

A solution of Example 1.27.1 (0.074 g), 2-(3H-[1,2,3]triazolo[4,5-b]pyridin-3-yl)-1,1,3,3-tetramethylisouronium hexafluorophosphate(V) (0.038 g), N,N-diisopropylethylamine (0.048 mL) and (R)-4-(tert-butoxy)-2-((tert-butoxycarbonyl)amino)-4-oxobutanoic acid (0.029 g) in dichloromethane (1 mL) was stirred for 2 hours. Trifluoroacetic acid (0.5 mL) was added, and stirring was continued overnight. The reaction was concentrated, dissolved in N,N-dimethylformamide (1.5 mL) and water (0.5 mL), and purified by reverse phase HPLC using a Gilson system, eluting with 10-75% acetonitrile in water containing 0.1% v/v trifluoroacetic acid. The desired fractions were combined and freeze-dried to provide the title compound. ¹H NMR (500 MHz, dimethyl sulfoxide-d₆) δ ppm 12.88 (s, 1H), 8.16 (s, 3H), 8.04 (d, 1H), 7.80 (d, 1H), 7.62 (d, 1H), 7.55-7.42 (m, 3H), 7.41-7.33 (m, 2H), 7.33-7.27 (m, 1H), 6.96 (d, 1H), 4.96 (s, 2H), 4.63-4.49 (m, 1H), 3.89 (t, 2H), 3.82 (s, 2H), 3.61-3.37 (m, 4H), 3.10-2.97 (m, 4H), 2.89-2.73 (m, 2H), 2.67-2.52 (m, 1H), 2.10 (s, 3H), 1.45-0.95 (m, 12H), 0.85 (s, 6H). MS (ESI) m/e 875.3 (M+H)⁺.

### 1.27. Synthesis of 6-[8-(1,3-benzothiazol-2-ylcarbamoyl)-3,4-dihydroisoquinolin-2(1H)-yl]-3-(1-{[3-(2-{[1-(carboxymethyl)piperidin-4-yl]amino}ethoxy)-5,7-dimethyltricyclo[3.3.1.1^{3,7}]dec-1-yl]methyl}-5-methyl-1H-pyrazol-4-yl)pyridine-2-carboxylic Acid (Compound W2.28)

A solution of Example 1.2.7 (0.055 g,), tert-butyl 2-(4-oxopiperidin-1-yl)acetate (0.014 g) and sodium triacetoxyborohydride (0.019 g) was stirred in dichloromethane (0.5 mL) at room temperature. After stirring for 2 hours, trifluoroacetic acid (0.5 mL) was added to the reaction, and stirring was continued overnight. The reaction was concentrated, dissolved in N,N-dimethylformamide (1.5 mL) and water (0.5 mL) and purified by reverse phase HPLC using a Gilson system, eluting with 10-80% acetonitrile in water containing 0.1% v/v trifluoroacetic acid. The desired fractions were combined and freeze-dried to provide the title compound. ¹H NMR (501 MHz, dimethyl sulfoxide-d₆) δ ppm 12.85 (s, 1H), 8.80 (s, 2H), 8.03 (d, 1H), 7.80 (d, 1H), 7.62 (d, 1H), 7.55-7.41 (m, 3H), 7.36 (q, 2H), 7.29 (s, 1H), 6.96 (d, 1H), 4.96 (s, 2H), 4.07 (s, 2H), 3.89 (t, 2H), 3.83 (s, 2H), 3.66-3.55 (m, 4H), 3.30 (s, 1H), 3.08 (s, 4H), 3.02 (t, 2H), 2.22 (d, 2H), 2.10 (s, 3H), 1.97-1.78 (m, 2H), 1.44 (s, 2H), 1.31 (q, 4H), 1.20-0.96 (m, 6H), 0.87 (s, 6H). MS (ESI) m/e 887.3 (M+H)⁺.

### 1.28. Synthesis of N-[(5S)-5-amino-6-{[2-({3-[(4-{6-[8-(1,3-benzothiazol-2-ylcarbamoyl)-3,4-dihydroisoquinolin-2(1H)-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](methyl)amino}-6-oxohexyl]-N,N-dimethylmethanaminium (Compound W2.29)

A solution of Fmoc-N-ε-(trimethyl)-L-lysine hydrochloride (0.032 g), 2-(3H-[1,2,3]triazolo[4,5-b]pyridin-3-yl)-1,1,3,3-tetramethylisouronium hexafluorophosphate(V) (0.028 g) and N,N-diisopropylethylamine (0.034 mL) in N,N-dimethylformamide (0.5 mL) was stirred for 5 minutes. The reaction was added to Example 1.13.7 (0.050 g), and stirring was continued at room temperature overnight. Diethylamine (0.069 mL) was added to the reaction, and stirring was continued for an additional 2 hours. The reaction was diluted with N,N-dimethylformamide (1 mL), water (0.5 mL), and trifluoroacetic acid (0.101 mL). The mixture was purified by reverse phase HPLC using a Gilson system, eluting with 10-90% acetonitrile in water containing 0.1% v/v trifluoroacetic acid. The desired fractions were combined and freeze-dried to provide the title compound. ¹H NMR (500 MHz, dimethyl sulfoxide-d₆) δ ppm 12.87 (s, 1H), 8.13 (s, 3H), 8.04 (d, 1H), 7.80 (d, 1H), 7.62 (d, 1H), 7.54-7.42 (m, 3H), 7.42-7.34 (m, 2H), 7.29 (s, 1H), 6.96 (d, 1H), 4.96 (s, 2H), 4.42-4.24 (m, 1H), 3.89 (t, 2H), 3.82 (s, 2H), 3.29-3.16 (m, 2H), 3.08-3.00 (m, 15H), 2.87 (s, 2H), 2.10 (s, 3H), 1.84-1.60 (m, 4H), 1.42-0.97 (m, 15H), 0.85 (s, 6H). MS (ESI) m/e 930.3 (M+H)⁺.

### 1.29. Synthesis of 6-[8-(1,3-benzothiazol-2-ylcarbamoyl)-3,4-dihydroisoquinolin-2(1H)-yl]-3-{1-[(3,5-dimethyl-7-{2-[piperidin-4-yl(2-sulfoethyl)amino]ethoxy}tricyclo[3.3.1.1^{3,7}]dec-1-yl)methyl]-5-methyl-1H-pyrazol-4-yl}pyridine-2-carboxylic Acid (Compound W2.30)

### 1.29.1. tert-butyl 6-[8-(1,3-benzothiazol-2-ylcarbamoyl)-3,4-dihydroisoquinolin-2(1H)-yl]-3-(1-{[3-({13-[1-(tert-butoxycarbonyl)piperidin-4-yl]-2,2,7,7-tetramethyl-10,10-dioxido-3,3-diphenyl-4,9-dioxa-102-thia-13-aza-3-silapentadecan-15-yl}oxy)-5,7-dimethyltricyclo[3.3.1.1^{3,7}]dec-1-yl]methyl}-5-methyl-1H-pyrazol-4-yl)pyridine-2-carboxylate

A solution of Example 1.2.8 (0.111 g), tert-butyl 4-oxopiperidine-1-carboxylate (0.021 g) and sodium triacetoxyborohydride (0.028 g) in dichloromethane (1 mL) was stirred at room temperature for 1 hour. Acetic acid (7.63 μL) was added, and stirring was continued overnight. Additional tert-butyl 4-oxopiperidine-1-carboxylate (0.021 g), sodium triacetoxyborohydride (0.028 g) and acetic acid (8 μL) were added to the reaction, and stirring was continued for an additional 4 hours. The reaction was loaded directly onto silica gel and eluted with a gradient of 0.5-4% methanol in dichloromethane to give the title compound.

### 1.29.2. 6-[8-(1,3-benzothiazol-2-ylcarbamoyl)-3,4-dihydroisoquinolin-2(1H)-yl]-3-{1-[(3,5-dimethyl-7-{2-[piperidin-4-yl(2-sulfoethyl)amino]ethoxy}tricyclo[3.3.1.1^{3,7}]dec-1-yl)methyl]-5-methyl-1H-pyrazol-4-yl}pyridine-2-carboxylic Acid

To a solution of Example 1.30.1 (0.078 g) in dichloromethane (1 mL) was added trifluoroacetic acid (0.5 mL), and the reaction was stirred at room temperature overnight. The reaction was concentrated and dissolved in N,N-dimethylformamide (1.5 mL) and water (0.5 mL). The mixture was purified by reverse phase HPLC using a Gilson system, eluting with 10-75% acetonitrile in water containing 0.1% v/v trifluoroacetic acid. The desired fractions were combined and freeze-dried to provide the title compound. ¹H NMR (400 MHz, dimethyl sulfoxide-d₆) δ ppm 12.89 (s, 1H), 9.31 (s, 1H), 8.75 (d, 1H), 8.36-8.19 (m, 1H), 8.08 (d, 1H), 7.84 (d, 1H), 7.66 (d, 1H), 7.58 (d, 1H), 7.55-7.45 (m, 2H), 7.40 (td, 2H), 7.34 (s, 1H), 6.99 (d, 1H), 5.00 (s, 2H), 3.93 (t, 2H), 3.87 (s, 2H), 3.49 (d, 6H), 3.39-3.31 (m, 2H), 3.01 (m, 6H), 2.15 (s, 6H), 1.94 (s, 2H), 1.58-0.99 (m, 12H), 0.91 (s, 6H). MS (ESI) m/e 937.3 (M+H)⁺.

### 1.30. Synthesis of 6-[8-(1,3-benzothiazol-2-ylcarbamoyl)-5-(3-phosphonopropoxy)-3,4-dihydroisoquinolin-2(1H)-yl]-3-[1-({3,5-dimethyl-7-[2-(methylamino)ethoxy]tricyclo[3.3.1.1^{3,7}]dec-1-yl}methyl)-5-methyl-1H-pyrazol-4-yl]pyridine-2-carboxylic Acid (Compound W2.31)

### 1.30.1. tert-butyl 8-bromo-5-hydroxy-3,4-dihydroisoquinoline-2(1H)-carboxylate

To a solution of tert-butyl 5-hydroxy-3,4-dihydroisoquinoline-2(1H)-carboxylate (9 g) in N,N-dimethylformamide (150 mL) was added N-bromosuccinimide (6.43 g). The mixture was stirred overnight and quenched with water (200 mL). The mixture was diluted with ethyl acetate (500 mL), washed with water and brine, and dried over sodium sulfate. Evaporation of the solvent gave the title compound, which was used in the next reaction without further purification. MS(ESI) m/e 329.2 (M+H)⁺.

### 1.30.2. tert-butyl 5-(benzyloxy)-8-bromo-3,4-dihydroisoquinoline-2(1H)-carboxylate

To a solution of Example 1.31.1 (11.8 g) in acetone (200 mL) was added benzyl bromide (7.42 g) and K₂CO₃ (5 g), and the mixture was stirred at reflux overnight. The mixture was concentrated, and the residue was partitioned between ethyl acetate (600 mL) and water (200 mL). The organic layer was washed with water and brine, dried over sodium sulfate, filtered and concentrated. The residue was purified by silica gel chromatography, eluting with 10% ethyl acetate in heptane, to provide the title compound. MS (ESI) m/e 418.1 (M+H)⁺.

### 1.30.3. 2-tert-butyl 8-methyl 5-(benzyloxy)-3,4-dihydroisoquinoline-2,8(1H)-dicarboxylate

Methanol (100 mL) and triethylamine (9.15 mL) were added to Example 1.31.2 (10.8 g) and [1,1′-bis(diphenylphosphino)ferrocene]dichloropalladium(II) (0.48 g) in a 500 mL stainless steel pressure reactor. The vessel was sparged with argon several times. The reactor was pressurized with carbon monoxide and stirred for 2 hours at 100° C. under 60 psi of carbon monoxide. After cooling, the crude reaction mixture was concentrated under vacuum. The residue was added to ethyl acetate (500 mL) and water (200 mL). The organic layer was further washed with water and brine, dried over sodium sulfate, filtered and concentrated. The residue was purified by silica gel chromatography, eluting with 10-20% ethyl acetate in heptane, to provide the title compound. MS (ESI) m/e 398.1 (M+H)⁺.

### 1.30.4. Methyl 5-(benzyloxy)-1,2,3,4-tetrahydroisoquinoline-8-carboxylate Hydrochloride

To a solution of Example 1.31.3 (3.78 g) in tetrahydrofuran (20 mL) was added 4N HCl in 1,4-dioxane (20 mL), and the mixture was stirred overnight. The mixture was concentrated under vacuum to give the title compound, which was used in the next reaction without further purification. MS(ESI) m/e 298.1 (M+H)⁺.

### 1.30.5. Methyl 5-(benzyloxy)-2-(5-bromo-6-(tert-butoxycarbonyl)pyridin-2-yl)-1,2,3,4-tetrahydroisoquinoline-8-carboxylate

To a solution of Example 1.31.4 (3.03 g) in dimethyl sulfoxide (50 mL) was added Example 1.1.10 (2.52 g) and triethylamine (3.8 mL), and the mixture was stirred at 60° C. overnight under nitrogen. The reaction mixture was diluted with ethyl acetate (500 mL), washed with water and brine, dried over sodium sulfate, filtered and concentrated. The residue was purified by silica gel chromatography, eluting with 20% ethyl acetate in heptane, to give the title compound. MS (ESI) m/e 553.1 (M+H)⁺.

### 1.30.6. tert-butyl (2-((3,5-dimethyl-7-((5-methyl-4-(4,4,5,5-tetramethyl-1,3,2-dioxaborolan-2-yl)-1H-pyrazol-1-yl)methyl)adamantan-1-yl)oxy)ethyl)(methyl)carbamate

To a solution of Example 1.13.3 (2.6 g) and [1,1′-bis(diphenylphosphino)ferrocene]dichloropalladium(II) dichloromethane (190 mg) in acetonitrile (30 mL) was added triethylamine (2.0 mL) and pinacolborane (1.4 mL), and the mixture was stirred at reflux overnight. The mixture was used directly in the next reaction without work up. MS (ESI) m/e 558.4 (M+H)⁺.

### 1.30.7. Methyl 5-(benzyloxy)-2-(6-(tert-butoxycarbonyl)-5-(1-((3-(2-((tert-butoxycarbonyl)(methyl)amino)ethoxy)-5,7-dimethyladamantan-1-yl)methyl)-5-methyl-1H-pyrazol-4-yl)pyridin-2-yl)-1,2,3,4-tetrahydroisoquinoline-8-carboxylate

To a solution of Example 1.31.5 (2.58 g) in tetrahydrofuran (40 mL) and water (20 mL) was added Example 1.31.6 (2.66 g), 1,3,5,7-tetramethyl-6-phenyl-2,4,8-trioxa-6-phosphaadamantane (341 mg), tris(dibenzylideneacetone)dipalladium(0) (214 mg), and K₃PO₄ (4.95 g), and the mixture was stirred at reflux for 4 hours. The mixture was diluted with ethyl acetate (500 mL), washed with water and brine, dried over sodium sulfate, filtered and concentrated. The residue was purified by silica gel chromatography, eluting with 20% ethyl acetate in dichloromethane, to provide the title compound. MS (ESI) m/e 904.5 (M+H)⁺.

### 1.30.8. Methyl 2-(6-(tert-butoxycarbonyl)-5-(1-((3-(2-((tert-butoxycarbonyl)(methyl)amino)ethoxy)-5,7-dimethyladamantan-1-yl)methyl)-5-methyl-1H-pyrazol-4-yl)pyridin-2-yl)-5-hydroxy-1,2,3,4-tetrahydroisoquinoline-8-carboxylate

Example 1.31.7 (3.0 g) in tetrahydrofuran (60 mL) was added to Pd(OH)₂ (0.6 g, Degussa #E101NE/W, 20% on carbon, 49% water content) in a 250 mL stainless steel pressure bottle. The mixture was shaken for 16 hours under 30 psi of hydrogen gas at 50° C. The mixture was filtered through a nylon membrane, and the solvent was evaporated under vacuum to provide the title compound. MS (ESI) m/e 815.1 (M+H)⁺.

### 1.30.9. Methyl 2-(6-(tert-butoxycarbonyl)-5-(1-((3-(2-((tert-butoxycarbonyl)(methyl)amino)ethoxy)-5,7-dimethyladamantan-1-yl)methyl)-5-methyl-1H-pyrazol-4-yl)pyridin-2-yl)-5-(3-(di-tert-butoxyphosphoryl)propoxy)-1,2,3,4-tetrahydroisoquinoline-8-carboxylate

To a solution of Example 1.31.8 (163 mg) in tetrahydrofuran (10 mL) was added Example 1.14.1 (50.5 mg), triphenylphosphine (52.5 mg) and di-tert-butylazodicarboxylate (46.2 mg), and the mixture was stirred for 3 hours. The mixture was diluted with ethyl acetate (200 mL), washed with water and brine, dried over sodium sulfate, filtered and concentrated. The residue was purified by silica gel chromatography, eluting with 20% ethyl acetate in heptanes followed by 5% methanol in dichloromethane, to provide the title compound. MS (ESI) m/e 1049.2 (M+H)⁺.

### 1.30.10.2-(6-(tert-butoxycarbonyl)-5-(1-((3-(2-((tert-butoxycarbonyl)(methyl)amino)ethoxy)-5,7-dimethyladamantan-1-yl)methyl)-5-methyl-1H-pyrazol-4-yl)pyridin-2-yl)-5-(3-(di-tert-butoxyphosphoryl)propoxy)-1,2,3,4-tetrahydroisoquinoline-8-carboxylic Acid

To a solution of Example 1.31.9 (3 g) in tetrahydrofuran (20 mL), methanol (10 mL) and water (10 mL) was added lithium hydroxide monohydrate (30 mg), and the mixture was stirred at room temperature for 24 hours. The reaction mixture was neutralized with 2% aqueous HCl and concentrated under vacuum. The residue was diluted with ethyl acetate (800 mL), washed with water and brine, and dried over sodium sulfate. Filtration and evaporation of solvent provided the title compound. MS (ESI) m/e 1034.5 (M+H)⁺.

### 1.30.11.6-[8-(1,3-benzothiazol-2-ylcarbamoyl)-5-(3-phosphonopropoxy)-3,4-dihydroisoquinolin-2(1H)-yl]-3-[1-({3,5-dimethyl-7-[2-(methylamino)ethoxy]tricyclo[3.3.1.1^{3,7}]dec-1-yl}methyl)-5-methyl-1H-pyrazol-4-yl]pyridine-2-carboxylic Acid

To a solution of Example 1.31.10 (207 mg) in N,N-dimethylformamide (4 mL) was added benzo[d]thiazol-2-amine (45.1 mg, 0.3 mmol), fluoro-N,N,N′,N′-tetramethylformamidinium hexafluorophosphate (79 mg) and N,N-diisopropylethylamine (150 mg), and the mixture was stirred at 60° C. for 3 hours. The reaction mixture was diluted with ethyl acetate (200 mL,) washed with water and brine, dried over sodium sulfate, filtered and concentrated. The residue was purified by silica gel chromatography, eluting with 20% ethyl acetate in heptane followed by 5% methanol in dichloromethane. After concentration, the material was dissolved in a mixture of dichloromethane and trifluoroacetic acid (1:1, 6 mL) and was allowed to sit at room temperature overnight. The solvent was evaporated, and the residue was dissolved in dimethyl sulfoxide/methanol (1:1, 9 mL). The mixture was purified by reverse phase HPLC using a Gilson system, eluting with 10-85% acetonitrile in water containing 0.1% v/v trifluoroacetic acid, to give the title compound. ¹H NMR (501 MHz, dimethyl sulfoxide-d₆) δ ppm 8.27 (s, 2H), 8.02 (d, 1H), 7.76 (dd, 2H), 7.43-7.56 (m, 2H), 7.32-7.37 (m, 1H), 7.29 (s, 1H), 7.00 (dd, 2H), 5.02 (s, 2H), 4.15 (t, 2H), 3.88-3.93 (m, 2H), 3.83 (s, 3H), 3.50-3.59 (m, 4H), 2.95-3.08 (m, 2H), 2.78-2.87 (m, 2H), 2.51-2.55 (m, 3H), 2.11 (s, 3H), 1.90-2.01 (m, 2H), 1.65-1.75 (m, 2H), 1.41 (s, 2H), 1.22-1.36 (m, 6H), 0.98-1.18 (m, 6H), 0.87 (s, 6H). MS (ESI) m/e 898.2 (M+H)⁺.

### 1.31. Synthesis of 6-[8-(1,3-benzothiazol-2-ylcarbamoyl)-3,4-dihydroisoquinolin-2(1H)-yl]-3-(1-{[3-(2-{[N-(2-carboxyethyl)-L-alpha-aspartyl]amino}ethoxy)-5,7-dimethyltricyclo[3.3.1.1^{3,7}]dec-1-yl]methyl}-5-methyl-1H-pyrazol-4-yl)pyridine-2-carboxylic Acid (Compound W2.32)

### 1.31.1. tert-butyl 6-(8-(benzo[d]thiazol-2-ylcarbamoyl)-3,4-dihydroisoquinolin-2(1H)-yl)-3-(1-((3-(2-((S)-4-(tert-butoxy)-2-((tert-butoxycarbonyl)amino)-4-oxobutanamido)ethoxy)-5,7-dimethyladamantan-1-yl)methyl)-5-methyl-1H-pyrazol-4-yl)picolinate

To a cold (0° C.) solution of (S)-4-(tert-butoxy)-2-((tert-butoxycarbonyl)amino)-4-oxobutanoic acid (136 mg) and O-(7-azabenzotriazol-1-yl)-N,N,N′,N′-tetramethyluronium hexafluorophosphate (HATU, 179 mg) in N,N-dimethylformamide (3 mL) was added N,N-diisopropylethylamine (165 μL). The reaction mixture was stirred for 10 minutes, and Example 1.2.7 (252 mg) in N,N-dimethylformamide (1 mL) was added. The mixture was stirred at room temperature for 1.5 hours and was purified by reverse phase chromatography (C18 column), eluting with 50-100% acetonitrile in water containing 0.1% v/v trifluoroacetic acid, to provide the title compound.

### 1.31.2. 3-(1-((3-(2-((S)-2-amino-3-carboxypropanamido)ethoxy)-5,7-dimethyladamantan-1-yl)methyl)-5-methyl-1H-pyrazol-4-yl)-6-(8-(benzo[d]thiazol-2-ylcarbamoyl)-3,4-dihydroisoquinolin-2(1H)-yl)picolinic Acid

Example 1.32.1 (100 mg) in dichloromethane (3 mL) was treated with trifluoroacetic acid (2.5 mL) overnight. The reaction mixture was concentrated to provide the title compound.

### 1.31.3. 6-(8-(benzo[d]thiazol-2-ylcarbamoyl)-3,4-dihydroisoquinolin-2(1H)-yl)-3-(1-((3-(2-((S)-2-((3-(tert-butoxy)-3-oxopropyl)amino)-3-carboxypropanamido)ethoxy)-5,7-dimethyladamantan-1-yl)methyl)-5-methyl-1H-pyrazol-4-yl)picolinic Acid

To a mixture of Example 1.32.2 (102 mg) and N,N-diisopropylethylamine (0.21 mL) in N,N-dimethylformamide (1.5 mL) was added tert-butyl acrylate (80 mg) and water (1.5 mL). The mixture was heated at 50° C. for 24 hours and purified by reverse phase chromatography (C18 column), eluting with 20-60% acetonitrile in water containing 0.1% v/v trifluoroacetic acid, to provide the title compound. MS (APCI) m/e 989.1 (M+H)⁺.

### 1.31.4. 6-[8-(1,3-benzothiazol-2-ylcarbamoyl)-3,4-dihydroisoquinolin-2(1H)-yl]-3-(1-{[3-(2-{[N-(2-carboxyethyl)-L-alpha-aspartyl]amino}ethoxy)-5,7-dimethyltricyclo[3.3.1.1^{3,7}]dec-1-yl]methyl}-5-methyl-1H-pyrazol-4-yl)pyridine-2-carboxylic Acid

The title compound was prepared as described in Example 1.6.2, replacing Example 1.6.1 with Example 1.32.3. ¹H NMR (400 MHz, dimethyl sulfoxide-d₆) δ ppm 12.86 (s, 3H), 8.62-9.21 (m, 2H), 8.52 (t, 1H), 8.03 (d, 1H), 7.79 (d, 1H), 7.62 (d, 1H), 7.42-7.53 (m, 3H), 7.33-7.41 (m, 2H), 7.29 (s, 1H), 6.95 (d, 1H), 4.96 (s, 2H), 4.04-4.19 (m, 1H), 3.89 (t, 2H), 3.81 (s, 2H), 3.32-3.41 (m, 2H), 3.16-3.27 (m, 2H), 3.10 (t, 2H), 3.01 (t, 2H), 2.83 (d, 2H), 2.66 (t, 2H), 2.10 (s, 3H), 1.39 (s, 2H), 1.20-1.32 (m, 4H), 0.94-1.16 (m, 6H), 0.85 (s, 6H). MS (ESI) m/e 933.2 (M+H)⁺.

### 1.32. Synthesis of 3-{1-[(3-{2-[(2-aminoethyl)(2-sulfoethyl)amino]ethoxy}-5,7-dimethyltricyclo[3.3.1.1^{3,7}]dec-1-yl)methyl]-5-methyl-1H-pyrazol-4-yl}-6-[8-(1,3-benzothiazol-2-ylcarbamoyl)-3,4-dihydroisoquinolin-2(1H)-yl]pyridine-2-carboxylic Acid (Compound W2.33)

### 1.32.1. 6-(8-(benzo[d]thiazol-2-ylcarbamoyl)-3,4-dihydroisoquinolin-2(1H)-yl)-3-(1-((3-(2-((2-((tert-butoxycarbonyl)amino)ethyl)(2-sulfoethyl)amino)ethoxy)-5,7-dimethyladamantan-1-yl)methyl)-5-methyl-1H-pyrazol-4-yl)picolinic Acid

To a solution of Example 1.2.9 (188 mg), tert-butyl (2-oxoethyl)carbamate (70.1 mg) and N,N-diisopropylethylamine (384 μL) was added sodium triacetoxyborohydride (140 mg), and the mixture was stirred overnight. NaCNBH₃ (13.83 mg) was added. The resulting mixture was stirred for 1 hour, and methanol (1 mL) was added. The mixture was stirred for 10 minutes, diluted with ethyl acetate, and washed with brine. The organic layer was dried over sodium sulfate, filtered and concentrated. The residue was purified by reverse phase chromatography (C18 column), eluting with 20-80% acetonitrile in water containing 0.1% v/v trifluoroacetic acid, to provide the title compound.

### 1.32.2. 3-{1-[(3-{2-[(2-aminoethyl)(2-sulfoethyl)amino]ethoxy}-5,7-dimethyltricyclo[3.3.1.1^{3,7}]dec-1-yl)methyl]-5-methyl-1H-pyrazol-4-yl}-6-[8-(1,3-benzothiazol-2-ylcarbamoyl)-3,4-dihydroisoquinolin-2(1H)-yl]pyridine-2-carboxylic Acid

The title compound was prepared as described in Example 1.6.2, replacing Example 1.6.1 with Example 1.33.1. ¹H NMR (400 MHz, dimethyl sulfoxide-d₆) δ ppm 12.85 (s, 1H), 8.03 (d, 1H), 7.87 (s, 2H), 7.79 (d, 1H), 7.62 (d, 1H), 7.41-7.56 (m, 3H), 7.33-7.40 (m, 2H), 7.29 (s, 1H), 6.96 (d, 1H), 4.96 (s, 2H), 3.89 (t, 2H), 3.50 (s, 2H), 3.29-3.40 (m, 4H), 3.19 (s, 2H), 3.01 (t, 2H), 2.94 (t, 2H), 2.11 (s, 3H), 1.43 (s, 2H), 1.25-1.37 (m, 4H), 0.98-1.19 (m, 6H), 0.87 (s, 6H). MS (ESI) m/e 897.2 (M+H)⁺.

### 1.33. Synthesis of 6-[5-(2-aminoethoxy)-8-(1,3-benzothiazol-2-ylcarbamoyl)-3,4-dihydroisoquinolin-2(1H)-yl]-3-[1-({3,5-dimethyl-7-[2-(methylamino)ethoxy]tricyclo[3.3.1.1^{3,7}]dec-1-yl}methyl)-5-methyl-1H-pyrazol-4-yl]pyridine-2-carboxylic Acid (Compound W2.34)

### 1.33.1. Methyl 5-(2-(((benzyloxy)carbonyl)amino)ethoxy)-2-(6-(tert-butoxycarbonyl)-5-(1-((3-(2-((tert-butoxycarbonyl)(methyl)amino)ethoxy)-5,7-dimethyladamantan-1-yl)methyl)-5-methyl-1H-pyrazol-4-yl)pyridin-2-yl)-1,2,3,4-tetrahydroisoquinoline-8-carboxylate

To a mixture of Example 1.31.8 (500 mg), benzyl (2-hydroxyethyl)carbamate (180 mg) and triphenyl phosphine (242 mg) in tetrahydrofuran (9 mL) was added (E)-di-tert-butyl diazene-1,2-dicarboxylate (212 mg). The mixture was stirred for 2 hours, diluted with ethyl acetate and washed with water and brine. The organic layer was dried over sodium sulfate, filtered, and concentrated. The residue was purified by silica gel chromatography, eluting with 50-100% ethyl acetate in heptanes, to provide the title compound. MS (APCI) m/e 991.1 (M+H)⁺.

### 1.33.2. 5-(2-(((benzyloxy)carbonyl)amino)ethoxy)-2-(6-(tert-butoxycarbonyl)-5-(1-((3-(2-((tert-butoxycarbonyl)(methyl)amino)ethoxy)-5,7-dimethyladamantan-1-yl)methyl)-5-methyl-1H-pyrazol-4-yl)pyridin-2-yl)-1,2,3,4-tetrahydroisoquinoline-8-carboxylic Acid

To a solution of Example 1.34.1 (480 mg) in tetrahydrofuran (10 mL) and methanol (5 mL) was added 1 M lithium hydroxide (1.94 mL). The mixture was heated at 50° C. overnight, cooled, acidified with 10% aqueous HCl to pH 3 and concentrated. The residue was purified by reverse phase chromatography (C18 column), eluting with 40-99% acetonitrile in water containing 0.1% v/v trifluoroacetic acid, to provide the title compound. MS (ESI) m/e 977.4 (M+H)⁺.

### 1.33.3. tert-butyl 6-(8-(benzo[d]thiazol-2-ylcarbamoyl)-5-(2-(((benzyloxy)carbonyl)amino)ethoxy)-3,4-dihydroisoquinolin-2(1H)-yl)-3-(1-((3-(2-((tert-butoxycarbonyl)(methyl)amino)ethoxy)-5,7-dimethyladamantan-1-yl)methyl)-5-methyl-1H-pyrazol-4-yl)picolinate

To a mixture of Example 1.34.2 (245 mg), benzo[d]thiazol-2-amine (151 mg) and fluoro-N,N,N′,N′-tetramethylformamidinium hexafluorophosphate (TFFH) (132 mg) in N,N-dimethylformamide (3 mL) was added N,N-diisopropylethylamine (876 μl). The reaction mixture was heated at 65° C. for 24 hours, cooled, diluted with ethyl acetate and washed with water and brine. The organic layer was dried over sodium sulfate, filtered and concentrated. The residue was purified by silica gel chromatography, eluting with 0-80% ethyl acetate in heptanes, to provide the title compound. MS (APCI) m/e 1109.5 (M+H)⁺.

### 1.33.4. 6-[5-(2-aminoethoxy)-8-(1,3-benzothiazol-2-ylcarbamoyl)-3,4-dihydroisoquinolin-2(1H)-yl]-3-[1-({3,5-dimethyl-7-[2-(methylamino)ethoxy]tricyclo[3.3.1.1^{3,7}]dec-1-yl}methyl)-5-methyl-1H-pyrazol-4-yl]pyridine-2-carboxylic Acid

Example 1.34.3 (100 mg) in dichloromethane (0.5 mL) was treated with trifluoroacetic acid (10 mL) overnight. The reaction mixture was concentrated and purified by reverse phase chromatography (C18 column), eluting with 20-60% acetonitrile in water containing 0.1% v/v trifluoroacetic acid, to provide the title compound. ¹H NMR (400 MHz, dimethyl sulfoxide-d₆) δ ppm 12.75 (s, 2H), 8.27 (s, 2H), 7.89-8.09 (m, 4H), 7.77 (s, 2H), 7.44-7.53 (m, 2H), 7.35 (t, 1H), 7.29 (s, 1H), 7.02 (dd, 2H), 5.02 (s, 2H), 4.27 (t, 2H), 3.87-3.97 (m, 2H), 3.83 (s, 2H), 3.50-3.58 (m, 2H), 3.00 (s, 2H), 2.88-2.96 (m, 2H), 2.52-2.60 (m, 2H), 2.10 (s, 3H), 1.42 (s, 2H), 1.23-1.36 (m, 4H), 0.98-1.19 (m, 6H), 0.87 (s, 6H). MS (ESI) m/e 819.3 (M+H)⁺.

### 1.34. Synthesis of 6-[8-(1,3-benzothiazol-2-ylcarbamoyl)naphthalen-2-yl]-3-{1-[(3,5-dimethyl-7-{2-[(3-sulfopropyl)amino]ethoxy}tricyclo[3.3.1.1^{3,7}]dec-1-yl)methyl]-5-methyl-1H-pyrazol-4-yl}pyridine-2-carboxylic Acid (Compound W2.35)

### 1.34.1. tert-butyl 6-chloro-3-(1-((3,5-dimethyl-7-(2-oxoethoxy)adamantan-1-yl)methyl)-5-methyl-1H-pyrazol-4-yl)picolinate

To a solution of oxalyl chloride (8 mL, 2.0 M in dichloromethane) in dichloromethane (20 mL) at −78° C., was added dropwise dimethyl sulfoxide (1 mL) in dichloromethane (10 mL) over 20 minutes. The solution was stirred for 30 minutes under argon, and Example 1.20.2 (3.8 g) as a solution in dichloromethane (30 mL) was added over 10 minutes. The reaction mixture was stirred at −78° C. for an additional 60 minutes. Triethylamine (2 mL) was added at −78° C., and the reaction mixture was stirred for 60 minutes. The cooling bath was removed, and the reaction allowed to warm to room temperature overnight. Water (60 mL) was added. The aqueous layer was acidified with 1% aqueous HCl solution and extracted with dichloromethane. The combined organic layers were washed with 1% aqueous HCl solution, aqueous NaHCO₃ solution, and brine. The organic layer was dried over sodium sulfate and concentrated to provide the title compound. MS (ESI) m/e 527.9 (M+H)⁺.

### 1.34.2. 2,2,2-trifluoro-1-(p-tolyl)ethyl 3-iodopropane-1-sulfonate

The title compound was prepared according to a procedure reported in J. Org. Chem., 2013, 78, 711-716.

### 1.34.3. 2,2,2-trifluoro-1-(p-tolyl)ethyl 3-aminopropane-1-sulfonate

A solution of Example 1.35.2 (2.0 g) in 7 N ammonia in methanol (20 mL) was heated to 80° C. under microwave conditions (Biotage Initiator) for 45 minutes. The mixture was concentrated, and the residue was dissolved in ethyl acetate (300 mL). The organic layer was washed with water and brine, dried over sodium sulfate, filtered, and concentrated to provide the title compound. MS (ESI) m/e 312.23 (M+H)⁺.

### 1.34.4. tert-butyl 6-chloro-3-(1-(((3,5-dimethyl-7-(2-((3-((2,2,2-trifluoro-1-(p-tolyl)ethoxy)sulfonyl)propyl)amino)ethoxy)adamantan-1-yl)methyl)-5-methyl-1H-pyrazol-4-yl)picolinate

To a solution of Example 1.35.3 (1.96 g) in dichloroethane (30 mL) was added Example 1.35.1 (3.33 g). The reaction mixture was stirred at room temperature for 1 hour, and a suspension of NaBH₄ (1.2 g) in methanol (8 mL) was added. The mixture was stirred at room temperature for 3 hours and diluted with ethyl acetate (300 mL). The organic layer was washed with 2N aqueous NaOH, water, and brine, dried over sodium sulfate, filtered and concentrated. The residue was dissolved in tetrahydrofuran (30 mL), and di-tert-butyl dicarbonate (2 g) was added followed by the addition of catalytic amount of 4-dimethylaminopyridine. The mixture was stirred at room temperature overnight. The mixture was diluted with ethyl acetate (300 mL) and washed with water and brine. The organic layer was dried over sodium sulfate, filtered, and concentrated to provide the title compound. MS (ESI) m/e 924.42 (M+H)⁺.

### 1.34.5. 7-(6-(tert-butoxycarbonyl)-5-(1-((3-(2-((tert-butoxycarbonyl)(3-((2,2,2-trifluoro-1-(p-tolyl)ethoxy)sulfonyl)propyl)amino)ethoxy)-5,7-dimethyladamantan-1-yl)methyl)-5-methyl-1H-pyrazol-4-yl)pyridin-2-yl)-1-naphthoic Acid

To a solution of methyl 7-(4,4,5,5-tetramethyl-1,3,2-dioxaborolan-2-yl)-1-naphthoate (203 mg) in a mixture of 1,4-dioxane (10 mL) and water (5 mL) was added Example 1.35.4 (600 mg), bis(triphenylphosphine)palladium(II)dichloride (45.6 mg), and cesium fluoride (296 mg). The mixture was heated at 120° C. under microwave conditions (Biotage Initiator) for 30 minutes, diluted with ethyl acetate (200 mL), and washed with water and brine. The organic layer was dried over sodium sulfate, filtered, and concentrated. The residue was purified by silica gel chromatography, eluting with 20% ethyl acetate in heptane, to provide an ester intermediate. The residue was dissolved in a mixture of tetrahydrofuran (8 mL), methanol (4 mL) and water (4 mL), and was treated with lithium hydroxide monohydrate (200 mg) for 3 hours. The reaction was acidified with 1N aqueous HCl to pH 4 and was diluted with ethyl acetate (400 mL). The resulting mixture was washed with water and brine. The organic layer was dried over sodium sulfate, filtered, and concentrated to provide the title compound. MS (ESI) m/e 1060.24 (M+H)⁺.

### 1.34.6. 6-[8-(1,3-benzothiazol-2-ylcarbamoyl)naphthalen-2-yl]-3-{1-[(3,5-dimethyl-7-{2-[(3-sulfopropyl)amino]ethoxy}tricyclo[3.3.1.1^{3,7}]dec-1-yl)methyl]-5-methyl-1H-pyrazol-4-yl}pyridine-2-carboxylic Acid

To a solution of Example 1.35.5 (405 mg) in dichloromethane (10 mL) was added benzo[d]thiazol-2-amine (57.4 mg), 1-ethyl-3-[3-(dimethylamino)propyl]-carbodiimide hydrochloride (146 mg) and 4-(dimethylamino)pyridine (93 mg). The mixture was stirred at room temperature overnight, diluted with ethyl acetate (200 mL), and washed with water and brine. The organic layer was dried over sodium sulfate, filtered, and concentrated. The residue was dissolved in dichloromethane (3 mL) and treated with trifluoroacetic acid (3 mL) overnight. The reaction mixture was concentrated, and the residue was purified by reverse phase HPLC (Gilson system), eluting with a gradient of 10-85% acetonitrile in water containing 0.1% v/v trifluoroacetic acid, to provide the title compound. ¹H NMR (400 MHz, dimethyl sulfoxide-d₆) δ ppm 13.08 (s, 1H), 9.00 (s, 1H), 8.53 (s, 2H), 8.36 (dd, 1H), 8.26-8.13 (m, 3H), 8.06 (dd, 1H), 8.04-7.97 (m, 1H), 7.94 (d, 1H), 7.80 (d, 1H), 7.69 (dd, 1H), 7.51-7.43 (m, 2H), 7.40-7.31 (m, 1H), 7.19 (d, 0H), 3.88 (s, 2H), 3.54 (t, 2H), 3.16-2.91 (m, 4H), 2.68-2.55 (m, 2H), 2.29 (s, 0H), 2.22 (s, 3H), 1.93 (p, 2H), 1.43 (s, 2H), 1.38-1.23 (m, 4H), 1.10 (dq, 6H), 0.87 (s, 6H). MS (ESI) m/e 863.2 (M+H)⁺.

### 1.35. Synthesis of 6-[8-(1,3-benzothiazol-2-ylcarbamoyl)-3,4-dihydroisoquinolin-2(1H)-yl]-3-{1-[(3-{2-[(2-carboxyethyl)(piperidin-4-yl)amino]ethoxy}-5,7-dimethyltricyclo[3.3.1.1^{3,7}]dec-1-yl)methyl]-5-methyl-1H-pyrazol-4-yl}pyridine-2-carboxylic Acid (Compound W2.36)

### 1.35.1. tert-butyl 6-(8-(benzo[d]thiazol-2-ylcarbamoyl)-3,4-dihydroisoquinolin-2(1H)-yl)-3-(1-(((1r,3r)-3-(2-((3-(tert-butoxy)-3-oxopropyl)(1-(tert-butoxycarbonyl)piperidin-4-yl)amino)ethoxy)-5,7-dimethyladamantan-1-yl)methyl)-5-methyl-1H-pyrazol-4-yl)picolinate

A solution of Example 1.25.1 (0.086 g), tert-butyl 4-oxopiperidine-1-carboxylate (0.037 g), sodium triacetoxyborohydride (0.039 g) and acetic acid (11 μl) in dichloromethane (1 mL) was stirred at room temperature. After stirring overnight, the reaction was loaded onto silica gel and eluted using a gradient of 0.5 to 5% methanol in dichloromethane to give the title compound. MS (ELSD) m/e 1113.5 (M+H)⁺.

### 1.35.2. 6-[8-(1,3-benzothiazol-2-ylcarbamoyl)-3,4-dihydroisoquinolin-2(1H)-yl]-3-{1-[(3-{2-[(2-carboxyethyl)(piperidin-4-yl)amino]ethoxy}-5,7-dimethyltricyclo[3.3.1.1^{3,7}]dec-1-yl)methyl]-5-methyl-1H-pyrazol-4-yl}pyridine-2-carboxylic Acid

A solution of Example 1.36.1 (0.050) in dichloromethane (0.5 mL) was treated with trifluoroacetic acid (0.5 mL), and the reaction was stirred overnight. The reaction was concentrated and dissolved in dimethyl sulfoxide and methanol (1:1). The mixture was purified by reverse phase HPLC using a Gilson system, eluting with 10-75% acetonitrile in water containing 0.1% v/v trifluoroacetic acid. The desired fractions were combined and freeze-dried to provide the title compound. ¹H NMR (400 MHz, dimethyl sulfoxide-d₆) δ ppm 12.84 (s, 1H), 9.38 (s, 1H), 8.78 (s, 1H), 8.42 (s, 1H), 8.03 (d, 1H), 7.80 (d, 1H), 7.63 (d, 1H), 7.55-7.42 (m, 3H), 7.41-7.33 (m, 2H), 7.30 (s, 1H), 6.96 (d, 1H), 4.96 (s, 2H), 3.89 (t, 2H), 3.83 (s, 2H), 3.73-3.54 (m, 3H), 3.53-3.34 (m, 4H), 3.34-3.25 (m, 2H), 3.02 (t, 2H), 2.99-2.85 (m, 2H), 2.78 (t, 2H), 2.23-2.04 (m, 5H), 1.92-1.76 (m, 2H), 1.43 (s, 2H), 1.39-1.23 (m, 4H), 1.23-0.96 (m, 6H), 0.87 (s, 6H). MS (ESI) m/e 901.3 (M+H)⁺.

### 1.36. Synthesis of 6-[8-(1,3-benzothiazol-2-ylcarbamoyl)-3,4-dihydroisoquinolin-2(1H)-yl]-3-{1-[(3,5-dimethyl-7-{2-[(3-sulfo-L-alanyl)(2-sulfoethyl)amino]ethoxy}tricyclo[3.3.1.1^{3,7}]dec-1-yl)methyl]-5-methyl-1H-pyrazol-4-yl}pyridine-2-carboxylic Acid (Compound W2.37)

A solution of (R)-2-((((9H-fluoren-9-yl)methoxy)carbonyl)amino)-3-sulfopropanoic acid (0.011 g) and 2-(3H-[1,2,3]triazolo[4,5-b]pyridin-3-yl)-1,1,3,3-tetramethylisouronium hexafluorophosphate(V) (10.80 mg) in N,N-dimethylformamide (0.5 mL) was stirred for 5 minutes. This solution was added to Example 1.2.9 (0.025 g) and N,N-diisopropylethylamine (0.014 mL). After stirring for 2 hours, diethylamine (0.013 mL) was added to the reaction, and stirring was continued for an additional 1 hour. The reaction was diluted with N,N-dimethylformamide and water and quenched with trifluoroacetic acid. The mixture was purified by reverse phase HPLC using a Gilson system, eluting with 10-75% acetonitrile in water containing 0.1% v/v trifluoroacetic acid. The desired fractions were combined and freeze-dried to provide the title compound. ¹H NMR (400 MHz, dimethyl sulfoxide-d₆) δ ppm 12.84 (s, 1H), 8.03 (dd, 4H), 7.79 (d, 1H), 7.62 (d, 1H), 7.54 (dd, 1H), 7.51-7.41 (m, 2H), 7.36 (td, 2H), 7.33 (s, 1H), 6.98 (dd, 1H), 4.96 (s, 2H), 4.42 (dd, 2H), 3.89 (t, 2H), 3.83 (s, 2H), 3.73 (ddd, 2H), 3.57-3.38 (m, 2H), 3.31 (dt, 1H), 3.08 (dd, 1H), 3.02 (t, 2H), 2.87 (tt, 1H), 2.81-2.54 (m, 2H), 2.10 (d, 3H), 1.51-0.91 (m, 12H), 0.85 (s, 6H). MS (ESI) m/e 1005.2 (M+H)⁺.

### 1.37. Synthesis of 6-[8-(1,3-benzothiazol-2-ylcarbamoyl)-3,4-dihydroisoquinolin-2(1H)-yl]-3-{1-[(3-{2-[{2-[(2-carboxyethyl)amino]ethyl}(2-sulfoethyl)amino]ethoxy}-5,7-dimethyltricyclo[3.3.1.1^{3,7}]dec-1-yl)methyl]-5-methyl-1H-pyrazol-4-yl}pyridine-2-carboxylic Acid (Compound W2.38)

### 1.37.1. 6-(S-(benzo[d]thiazol-2-ylcarbamoyl)-3,4-dihydroisoquinolin-2(1H)-yl)-3-(1-((3-(2-((2-((3-(tert-butoxy)-3-oxopropyl)amino)ethyl)(2-sulfoethyl)amino)ethoxy)-5,7-dimethyladamantan-1-yl)methyl)-5-methyl-1H-pyrazol-4-yl)picolinic Acid

The title compound was prepared as described in Example 1.32.3, replacing Example 1.32.2 with Example 1.33.2.

### 1.37.2. 6-[8-(1,3-benzothiazol-2-ylcarbamoyl)-3,4-dihydroisoquinolin-2(1H)-yl]-3-{1-[(3-{2-[{2-[(2-carboxyethyl)amino]ethyl}(2-sulfoethyl)amino]ethoxy}-5,7-dimethyltricyclo[3.3.1.1^{3,7}]dec-1-yl)methyl]-5-methyl-1H-pyrazol-4-yl}pyridine-2-carboxylic Acid

The title compound was prepared as described in Example 1.6.2, replacing Example 1.6.1 with Example 1.38.1. ¹H NMR (501 MHz, dimethyl sulfoxide-d₆) δ ppm 12.87 (s, 1H), 8.68 (s, 2H), 8.04 (d, 1H), 7.79 (d, 1H), 7.62 (d, 1H), 7.53 (d, 1H), 7.42-7.50 (m, 2H), 7.33-7.40 (m, 2H), 7.29 (s, 1H), 6.96 (d, 1H), 4.96 (s, 3H), 3.89 (t, 2H), 3.83 (s, 2H), 3.66 (t, 2H), 3.31-3.53 (m, 8H), 3.18 (t, 2H), 3.02 (t, 2H), 2.95 (t, 2H), 2.67 (t, 2H), 2.11 (s, 3H), 1.43 (s, 2H), 1.22-1.37 (m, 6H), 0.98-1.19 (m, 6H), 0.87 (s, 6H). MS (APCI) m/e 971.0 (M+H)⁺.

### 1.38. Synthesis of 3-{1-[(3,5-dimethyl-7-{2-[(3-phosphonopropyl)amino]ethoxy}tricyclo[3.3.1.1^{3,7}]dec-1-yl)methyl]-5-methyl-1H-pyrazol-4-yl}-6-[8-([1,3]thiazolo[4,5-b]pyridin-2-ylcarbamoyl)-3,4-dihydroisoquinolin-2(1H)-yl]pyridine-2-carboxylic Acid (Compound W2.39)

### 1.38.1. tert-butyl 3-(1-((3-(2-((3-(di-tert-butoxyphosphoryl)propyl)amino)ethoxy)-5,7-dimethyladamantan-1-yl)methyl)-5-methyl-1H-pyrazol-4-yl)-6-(8-(thiazolo[4,5-b]pyridin-2-ylcarbamoyl)-3,4-dihydroisoquinolin-2(1H)-yl)picolinate

Example 1.23.2 (520 mg) and Example 1.14.2 (175 mg) were dissolved in dichloromethane (6 mL) and stirred at room temperature for two hours. A suspension of sodium borohydride (32 mg) in methanol (1 mL) was added, and the mixture was stirred for 30 minutes. The reaction was added to saturated aqueous NaHCO₃ solution and extracted with ethyl acetate. The organic layer was washed with brine and dried over sodium sulfate. After filtration and concentration, purification by silica gel chromatography, eluting with a gradient of 0.5-5.0% methanol in dichloromethane, gave the title compound. MS (ESI) m/e 1037.3 (M+H)⁺.

### 1.38.2. 3-{1-[(3,5-dimethyl-7-{2-[(3-phosphonopropyl)amino]ethoxy}tricyclo[3.3.1.1^{3,7}]dec-1-yl)methyl]-5-methyl-1H-pyrazol-4-yl}-6-[8-([1,3]thiazolo[4,5-b]pyridin-2-ylcarbamoyl)-3,4-dihydroisoquinolin-2(1H)-yl]pyridine-2-carboxylic Acid

The title compound was prepared by substituting Example 1.39.1 for Example 1.2.8 in Example 1.2.9. ¹H NMR (400 MHz, dimethyl sulfoxide-d₆) δ ppm 8.60 (dd, 1H), 8.52 (dd, 1H), 8.41 (br s, 2H), 7.65 (d, 1H) 7.48 (d, 1H), 7.46 (d, 1H), 7.38 (m, 2H), 7.29 (s, 1H), 6.97 (d, 1H), 4.97 (s, 2H), 3.89 (m, 2H), 3.83 (s, 2H), 3.56 (m, 2H), 3.02 (m, 6H), 2.11 (s, 3H), 1.81 (m, 2H), 1.61 (m, 2H), 2.11 (s, 3H), 1.43 (s, 2H), 1.30 (m, 4H), 1.14 (m, 4H), 1.04 (m, 2H), 0.87 (s, 6H). MS (ESI) m/e 869.2 (M+H)⁺.

### 1.39. Synthesis of 3-{1-[(3,5-dimethyl-7-{2-[(3-phosphonopropyl)amino]ethoxy}tricyclo[3.3.1.1^{3,7}]dec-1-yl)methyl]-5-methyl-1H-pyrazol-4-yl}-6-[8-([1,3]thiazolo[5,4-b]pyridin-2-ylcarbamoyl)-3,4-dihydroisoquinolin-2(1H)-yl]pyridine-2-carboxylic Acid (Compound W2.40)

### 1.39.1. tert-butyl 3-(1-((3-(2-((3-(di-tert-butoxyphosphoryl)propyl)amino)ethoxy)-5,7-dimethyladamantan-1-yl)methyl)-5-methyl-1H-pyrazol-4-yl)-6-(8-(thiazolo[5,4-b]pyridin-2-ylcarbamoyl)-3,4-dihydroisoquinolin-2(1H)-yl)picolinate

The title compound was prepared by substituting Example 1.22.2 for Example 1.23.2 in Example 1.39.1. MS (ESI) m/e 1037.3 (M+H)⁺.

### 1.39.2. 3-{1-[(3,5-dimethyl-7-{2-[(3-phosphonopropyl)amino]ethoxy}tricyclo[3.3.1.1^{3,7}]dec-1-yl)methyl]-5-methyl-1H-pyrazol-4-yl}-6-[8-([1,3]thiazolo[5,4-b]pyridin-2-ylcarbamoyl)-3,4-dihydroisoquinolin-2(1H)-yl]pyridine-2-carboxylic Acid

The title compound was prepared by substituting Example 1.40.1 for Example 1.2.8 in Example 1.2.9. ¹H NMR (500 MHz, dimethyl sulfoxide-d₆) δ ppm 8.52 (dd, 2H), 8.41 (br s, 2H), 8.17 (dd, 1H), 7.63 (m, 1H), 7.53 (m, 2H), 7.46 (d, 1H), 7.38 (t, 1H), 7.30 (s, 1H), 6.98 (d, 1H), 4.96 (s, 2H), 3.88 (m, 2H), 3.83 (s, 2H), 3.56 (t, 2H), 3.00 (m, 6H), 2.11 (s, 3H), 1.81 (m, 2H), 1.60 (m, 2H), 1.43 (s, 2H), 1.31 (m, 4H), 1.14 (m, 4H), 1.04 (m, 2H), 0.87 (s, 6H). MS (ESI) m/e 869.2 (M+H)⁺.

### 1.40. Synthesis of 6-[8-(1,3-benzothiazol-2-ylcarbamoyl)-5-(carboxymethoxy)-3,4-dihydroisoquinolin-2(1H)-yl]-3-[1-({3,5-dimethyl-7-[2-(methylamino)ethoxy]tricyclo[3.3.1.1^{3,7}]dec-1-yl}methyl)-5-methyl-1H-pyrazol-4-yl]pyridine-2-carboxylic Acid (Compound W2.41)

### 1.40.1. Methyl 5-(2-(tert-butoxy)-2-oxoethoxy)-2-(6-(tert-butoxycarbonyl)-5-(1-((3-(2-((tert-butoxycarbonyl)(methyl)amino)ethoxy)-5,7-dimethyladamantan-1-yl)methyl)-5-methyl-1H-pyrazol-4-yl)pyridin-2-yl)-1,2,3,4-tetrahydroisoquinoline-8-carboxylate

To a solution of Example 1.31.8 (163 mg) in N,N-dimethylformamide (10 mL) was added tert-butyl 2-bromoacetate (58.6 mg), and K₂CO₃ (83 mg), and the reaction was stirred overnight. The mixture was diluted with ethyl acetate (200 mL), washed with water and brine, and dried over sodium sulfate. Filtration and evaporation of the solvent gave a residue that was purified by silica gel chromatography, eluting with 20% ethyl acetate in heptane, to provide the title compound. MS (ESI) m/e 929.2 (M+H)⁺.

### 1.40.2. 5-(2-(tert-butoxy)-2-oxoethoxy)-2-(6-(tert-butoxycarbonyl)-5-(1-((3-(2-((tert-butoxycarbonyl)(methyl)amino)ethoxy)-5,7-dimethyladamantan-1-yl)methyl)-5-methyl-1H-pyrazol-4-yl)pyridin-2-yl)-1,2,3,4-tetrahydroisoquinoline-8-carboxylic Acid

To a solution of Example 1.41.1 (3 g) in tetrahydrofuran (20 mL), methanol (10 mL) and water (10 mL) was added lithium hydroxide monohydrate (300 mg). The mixture was stirred at room temperature for 24 hours. The reaction mixture was neutralized with 2% aqueous HCl solution and concentrated under vacuum. The residue was diluted with ethyl acetate (800 mL), washed with water and brine, and dried over sodium sulfate. Filtration and evaporation of the solvent provided the title compound. MS (ESI) m/e 914.5 (M+H)⁺.

### 1.40.3. 6-[8-(1,3-benzothiazol-2-ylcarbamoyl)-5-(carboxymethoxy)-3,4-dihydroisoquinolin-2(1H)-yl]-3-[1-({3,5-dimethyl-7-[2-(methylamino)ethoxy]tricyclo[3.3.1.1^{3,7}]dec-1-yl}methyl)-5-methyl-1H-pyrazol-4-yl]pyridine-2-carboxylic Acid

To a solution of Example 1.41.2 (183 mg) in N,N-dimethylformamide (4 mL) was added benzo[d]thiazol-2-amine (45.1 mg), fluoro-N,N,N′,N′-tetramethylformamidinium hexafluorophosphate (79 mg) and N,N-diisopropylethylamine (0.203 mL). The mixture was stirred at 60° C. overnight. The mixture was diluted with ethyl acetate (300 mL), washed with water and brine, and dried over sodium sulfate. Filtration and evaporation of the solvent gave a residue that was dissolved in dichloromethane/trifluoroacetic acid (1:1, 10 mL) and stirred overnight. The mixture was concentrated, and the residue was purified by reverse phase HPLC using a Gilson system, eluting with 10-85% acetonitrile in in water containing 0.1% v/v trifluoroacetic acid, to provide the title compound. ¹H NMR (400 MHz, dimethyl sulfoxide-d₆) δ ppm 12.73 (s, 1H), 8.30 (s, 2H), 7.99-8.07 (m, 1H), 7.75-7.79 (m, 1H), 7.70 (d, 1H), 7.44-7.56 (m, 2H), 7.30-7.39 (m, 2H), 7.30 (s, 1H), 7.03 (t, 1H), 6.87-6.93 (m, 1H), 4.98-5.18 (m, 4H), 4.84 (s, 3H), 3.78-4.01 (m, 4H), 3.55 (t, 2H). 2.77-3.07 (m, 4H), 2.53-2.61 (m, 3H), 2.04-2.16 (m, 3H), 1.41 (s, 2H), 1.02-1.34 (m, 6H), 0.83-0.91 (m, 6H). MS (ESI) m/e 834.2 (M+H)⁺.

### 1.41. Synthesis of 6-[8-(1,3-benzothiazol-2-ylcarbamoyl)-3,4-dihydroisoquinolin-2(1H)-yl]-3-{1-[(3-{2-[(3-carboxypropyl)(piperidin-4-yl)amino]ethoxy}-5,7-dimethyltricyclo[3.3.1.1^{3,7}]dec-1-yl)methyl]-5-methyl-1H-pyrazol-4-yl}pyridine-2-carboxylic Acid (Compound W2.42)

### 1.41.1. tert-butyl 6-(8-(benzo[d]thiazol-2-ylcarbamoyl)-3,4-dihydroisoquinolin-2(1H)-yl)-3-(1-(((1r,3r)-3-(2-((1-(tert-butoxycarbonyl)piperidin-4-yl)(4-methoxy-4-oxobutyl)amino)ethoxy)-5,7-dimethyladamantan-1-yl)methyl)-5-methyl-1H-pyrazol-4-yl)picolinate

A solution of Example 1.26.1 (0.169 g), methyl 4-oxobutanoate (0.024 g) and sodium triacetoxyborohydride (0.055 g) was stirred in dichloromethane (2 mL) at room temperature. After 2 hours, the reaction was diluted with dichloromethane (50 mL) and washed with saturated aqueous sodium bicarbonate (10 mL). The organic layer was separated, dried over magnesium sulfate, filtered and concentrated. Silica gel chromatography, eluting with a gradient of 0.5-5% methanol/dichloromethane containing ammonia, provided the title compound. MS (ELSD) m/e 1085.5 (M+H)⁺.

### 1.41.2. 6-[8-(1,3-benzothiazol-2-ylcarbamoyl)-3,4-dihydroisoquinolin-2(1H)-yl]-3-{1-[(3-{2-[(3-carboxypropyl)(piperidin-4-yl)amino]ethoxy}-5,7-dimethyltricyclo[3.3.1.1^{3,7}]dec-1-yl)methyl]-5-methyl-1H-pyrazol-4-yl}pyridine-2-carboxylic Acid

A solution of Example 1.42.1 (0.161 g) in dichloromethane (0.5 mL) was treated with trifluoroacetic acid (0.5 mL), and the reaction was stirred overnight. The reaction was concentrated, dissolved in methanol (0.6 mL) and treated with lithium hydroxide monohydrate (0.124 g) as a solution in water (0.5 mL). After stirring for 1.5 hours, the reaction was quenched with trifluoroacetic acid (0.229 mL) and diluted with N,N-dimethylformamide (0.5 mL). The mixture was purified by reverse phase HPLC using a Gilson system, eluting with 10-60% acetonitrile in water containing 0.1% v/v trifluoroacetic acid. The desired fractions were combined and freeze-dried to provide the title compound. ¹H NMR (400 MHz, dimethyl sulfoxide-d₆) δ ppm 12.84 (s, 1H), 9.40 (s, 1H), 8.89-8.79 (m, 1H), 8.57-8.41 (m, 1H), 8.03 (d, 1H), 7.80 (d, 1H), 7.62 (d, 1H), 7.55-7.41 (m, 3H), 7.41-7.32 (m, 2H), 7.30 (s, 1H), 6.96 (d, 1H), 4.96 (s, 2H), 3.89 (t, 2H), 3.83 (s, 2H), 3.44 (d, 2H), 3.26 (s, 2H), 3.22-3.11 (m, 2H), 3.09-2.85 (m, 6H), 2.34 (t, 2H), 2.19 (d, 2H), 2.10 (s, 3H), 1.95-1.71 (m, 5H), 1.44 (s, 2H), 1.39-1.27 (m, 4H), 1.22-0.96 (m, 6H), 0.87 (s, 6H). MS (ESI) m/e 915.3 (M+H)⁺.

### 1.42. Synthesis of 6-[8-(1,3-benzothiazol-2-ylcarbamoyl)naphthalen-2-yl]-3-{1-[(3,5-dimethyl-7-{2-[(2-sulfoethyl)amino]ethoxy}tricyclo[3.3.1.1^{3,7}]dec-1-yl)methyl]-5-methyl-1H-pyrazol-4-yl}pyridine-2-carboxylic Acid (Compound W2.43)

### 1.42.1. tert-butyl 3-(1-((3-(2-hydroxyethoxy)-5,7-dimethyladamantan-1-yl)methyl)-5-methyl-1H-pyrazol-4-yl)-6-(8-(methoxycarbonyl)naphthalen-2-yl)picolinate

To a solution of methyl 7-(4,4,5,5-tetramethyl-1,3,2-dioxaborolan-2-yl)-1-naphthoate (2.47 g) in 1,4-dioxane (40 mL) and water (20 mL) was added Example 1.20.2 (4.2 g), bis(triphenylphosphine)palladium(II)dichloride (556 mg), and cesium fluoride (3.61 g), and the reaction was stirred at reflux overnight. The mixture was diluted with ethyl acetate (400 mL) and washed with water and brine, and dried over sodium sulfate. Filtration and evaporation of the solvent gave a residue that was purified by silica gel chromatography, eluting with 20% ethyl acetate in heptane followed by 5% methanol in dichloromethane, to provide the title compound. MS (ESI) m/e 680.7 (M+H)⁺.

### 1.42.2. tert-butyl 3-(1-((3,5-dimethyl-7-(2-((methylsulfonyl)oxy)ethoxy)adamantan-1-yl)methyl)-5-methyl-1H-pyrazol-4-yl)-6-(8-(methoxycarbonyl)naphthalen-2-yl)picolinate

To a cooled (0° C.) solution of Example 1.43.1 (725 mg) in dichloromethane (10 mL) and triethylamine (0.5 mL) was added methanesulfonyl chloride (0.249 mL), and the mixture was stirred for 4 hours. The reaction mixture was diluted with ethyl acetate (200 mL) and washed with water and brine, and dried over sodium sulfate. Filtration and evaporation of the solvent gave the title product, which was used in the next reaction without further purification. MS (ESI) m/e 759.9 (M+H)⁺.

### 1.42.3. tert-butyl 3-(1-(((3-(2-azidoethoxy)-5,7-dimethyladamantan-1-yl)methyl)-5-methyl-1H-pyrazol-4-yl)-6-(8-(methoxycarbonyl)naphthalen-2-yl)picolinate

To a solution of Example 1.43.2 (4.2 g) in N,N-dimethylformamide (30 mL) was added sodium azide (1.22 g), and the mixture was stirred for 96 hours. The reaction mixture was diluted with ethyl acetate (600 mL), washed with water and brine, and dried over sodium sulfate. Filtration and evaporation of the solvent provided the title compound. MS (ESI) m/e 705.8 (M+H)⁺.

### 1.42.4. 7-(5-(1-((3-(2-azidoethoxy)-5,7-dimethyladamantan-1-yl)methyl)-5-methyl-1H-pyrazol-4-yl)-6-(tert-butoxycarbonyl)pyridin-2-yl)-1-naphthoic Acid

To a solution of Example 1.43.3 (3.5 g) in tetrahydrofuran/methanol/water (2:1:1, 30 mL) was added lithium hydroxide monohydrate (1.2 g), and the mixture was stirred overnight. The reaction mixture was acidified with 1N aqueous HCl and was diluted with ethyl acetate (600 mL), washed with water and brine, and dried over sodium sulfate. Filtration and evaporation of the solvent provided the title compound. MS (ESI) m/e 691.8 (M+H)⁺.

### 1.42.5. tert-butyl 3-(1-((3-(2-azidoethoxy)-5,7-dimethyladamantan-1-yl)methyl)-5-methyl-1H-pyrazol-4-yl)-6-(8-(benzo[d]thiazol-2-ylcarbamoyl)naphthalen-2-yl)picolinate

To a solution of Example 1.43.5 (870 mg) in N,N-dimethylformamide (10 mL) was added benzo[d]thiazol-2-amine (284 mg), fluoro-N,N,N′,N′-tetramethylformamidinium hexafluorophosphate (499 mg) and N,N-diisopropylethylamine (488 mg). The mixture was stirred at 60° C. for 3 hours. The reaction mixture was diluted with ethyl acetate (200 mL) and washed with water and brine, and dried over sodium sulfate. Filtration and evaporation of the solvent provided the title compound. MS (ESI) m/e 824.1 (M+H)⁺.

### 1.42.6. tert-butyl 3-(1-((3-(2-aminoethoxy)-5,7-dimethyladamantan-1-yl)methyl)-5-methyl-1H-pyrazol-4-yl)-6-(8-(benzo[d]thiazol-2-ylcarbamoyl)naphthalen-2-yl)picolinate

To a solution of Example 1.43.5 (890 mg) in tetrahydrofuran (30 mL) was added Pd/C (90 mg). The mixture was stirred under 1 atmosphere of hydrogen overnight. The reaction mixture was filtered, and the catalyst was washed with ethyl acetate. The solvent was evaporated to provide the title compound. MS (ESI) m/e 798.1 (M+H)⁺.

### 1.42.7. 6-[8-(1,3-benzothiazol-2-ylcarbamoyl)naphthalen-2-yl]-3-{1-[(3,5-dimethyl-7-{2-[(2-sulfoethyl)amino]ethoxy}tricyclo[3.3.1.1^{3,7}]dec-1-yl)methyl]-5-methyl-1H-pyrazol-4-yl}pyridine-2-carboxylic Acid

To a solution of Example 1.43.6 (189 mg) in N,N-dimethylformamide (6 mL) was added 4-((tert-butyldiphenylsilyl)oxy)-2,2-dimethylbutyl ethenesulfonate (106 mg). The mixture was stirred for 4 days. The mixture was diluted with ethyl acetate (300 mL) and washed with water and brine and dried over sodium sulfate. After filtration and evaporation of the solvent, the residue was dissolved in trifluoroacetic acid (10 mL) and sat overnight. The trifluoroacetic acid was evaporated under vacuum, and the residue was dissolved in dimethyl sulfoxide/methanol (1:1, 6 mL). The mixture was purified by reverse phase HPLC (Gilson system), eluting with 10-85% acetonitrile in water containing 0.1% v/v trifluoroacetic acid, to give the title compound. ¹H NMR (400 MHz, dimethyl sulfoxide-d₆) δ ppm 13.09 (s, 1H), 9.02 (s, 1H), 8.31-8.43 (m, 3H), 8.16-8.26 (m, 3H), 7.93-8.08 (m, 3H), 7.82 (d, 1H), 7.66-7.75 (m, 1H), 7.46-7.55 (m, 2H), 7.37 (t, 1H), 3.90 (s, 3H), 3.17-3.28 (m, 2H), 3.07-3.16 (m, 2H), 2.82 (t, 2H), 2.24 (s, 3H), 1.44 (s, 2H), 0.99-1.37 (m, 12H), 0.87 (s, 6H). MS (ESI) m/e 849.1 (M+H)⁺.

### 1.43. Synthesis of 3-{1-[(3-{2-[L-alpha-aspartyl(2-sulfoethyl)amino]ethoxy}-5,7-dimethyltricyclo[3.3.1.1^{3,7}]dec-1-yl)methyl]-5-methyl-1H-pyrazol-4-yl}-6-[8-(1,3-benzothiazol-2-ylcarbamoyl)-3,4-dihydroisoquinolin-2(1H)-yl]pyridine-2-carboxylic Acid (Compound W2.44)

### 1.43.1. 6-(8-(benzo[d]thiazol-2-ylcarbamoyl)-3,4-dihydroisoquinolin-2(1H)-yl)-3-(1-((3-(2-((S)-4-(tert-butoxy)-2-((tert-butoxycarbonyl)amino)-4-oxo-N-(2-sulfoethyl)butanamido)ethoxy)-5,7-dimethyladamantan-1-yl)methyl)-5-methyl-1H-pyrazol-4-yl)picolinic Acid

To a cold (0° C.) solution of (S)-4-(tert-butoxy)-2-((tert-butoxycarbonyl)amino)-4-oxobutanoic acid (40.7 mg) and O-(7-azabenzotriazol-1-yl)-N,N,N′,N′-tetramethyluronium hexafluorophosphate (HATU, 40.1 mg,) in N,N-dimethylformamide (3 mL) was added N,N-diisopropylethylamine (98 μL). The reaction mixture was stirred at room temperature for 1 hour, and Example 1.2.9 (60 mg) in N,N-dimethylformamide (1 mL) was added. The mixture was stirred for 1.5 hours and was purified by reverse phase chromatography (C18 column), eluting with 20-90% acetonitrile in water containing 0.1% v/v trifluoroacetic acid, to provide the title compound. MS (ESI) m/e 1123.4 (M−H)⁻.

### 1.43.2. 3-{1-[(3-{2-[L-alpha-aspartyl(2-sulfoethyl)amino]ethoxy}-5,7-dimethyltricyclo[3.3.1.1^{3,7}]dec-1-yl)methyl]-5-methyl-1H-pyrazol-4-yl}-6-[8-(1,3-benzothiazol-2-ylcarbamoyl)-3,4-dihydroisoquinolin-2(1H)-yl]pyridine-2-carboxylic Acid

Example 1.44.1 (100 mg) in dichloromethane (5 mL) was treated with trifluoroacetic acid (1.5 mL) overnight. The reaction mixture was concentrated and purified by reverse phase chromatography (C18 column), eluting with 20-60% acetonitrile in water containing 0.1% v/v trifluoroacetic acid, to provide the title compound. ¹H NMR (500 MHz, dimethyl sulfoxide-d₆) δ ppm 12.85 (s, 2H), 8.11-8.22 (m, 3H), 8.04 (d, 1H), 7.79 (d, 1H), 7.62 (d, 1H), 7.41-7.54 (m, 3H), 7.32-7.39 (m, 2H), 7.29 (s, 1H), 6.95 (d, 1H), 4.95 (s, 2H), 4.80 (s, 1H), 3.89 (t, 2H), 3.81 (s, 2H), 3.55-3.71 (m, 2H), 3.01 (t, 4H), 2.74-2.86 (m, 1H), 2.57-2.73 (m, 2H), 2.09 (s, 3H), 0.91-1.46 (m, 13H), 0.84 (s, 6H). MS (ESI) m/e 969.2 (M+H)⁺.

### 1.44. Synthesis of 6-[8-(1,3-benzothiazol-2-ylcarbamoyl)-3,4-dihydroisoquinolin-2(1H)-yl]-3-{1-[(3-{2-[(1,3-dihydroxypropan-2-yl)amino]ethoxy}-5,7-dimethyltricyclo[3.3.1.1^{3,7}]dec-1-yl)methyl]-5-methyl-1H-pyrazol-4-yl}pyridine-2-carboxylic Acid (Compound W2.45)

### 1.44.1. tert-butyl 6-(8-(benzo[d]thiazol-2-ylcarbamoyl)-3,4-dihydroisoquinolin-2(1H)-yl)-3-(1-((3,5-dimethyl-7-(2-(oxetan-3-ylamino)ethoxy)adamantan-1-yl)methyl)-5-methyl-1H-pyrazol-4-yl)picolinate

A solution of Example 1.2.7 (0.095 g), oxetan-3-one (10 mg) and sodium triacetoxyborohydride (0.038 g) was stirred in dichloromethane (1 mL) at room temperature. After stirring overnight, the reaction mixture was loaded directly onto silica gel and eluted using a gradient of 0.5-5% methanol in dichloromethane containing ammonia to give the title compound. MS (ELSD) m/e 858.4 (M+H)⁺.

### 1.44.2. 6-[8-(1,3-benzothiazol-2-ylcarbamoyl)-3,4-dihydroisoquinolin-2(1H)-yl]-3-{1-[(3-{2-[(1,3-dihydroxypropan-2-yl)amino]ethoxy}-5,7-dimethyltricyclo[3.3.1.1^{3,7}]dec-1-yl)methyl]-5-methyl-1H-pyrazol-4-yl}pyridine-2-carboxylic Acid

Example 1.45.1 was dissolved in dichloromethane (0.5 mL) and was treated with trifluoroacetic acid (0.5 mL) and stirred overnight. The reaction was purified by reverse phase HPLC using a Gilson system, eluting with 10-60% acetonitrile in water containing 0.1% v/v trifluoroacetic acid. The desired fractions were combined and freeze-dried to provide the title compound. ¹H NMR (400 MHz, dimethyl sulfoxide-d₆) δ ppm 12.84 (s, 1H), 8.19 (s, 2H), 8.02 (d, 1H), 7.78 (d, 1H), 7.61 (d, 1H), 7.53-7.40 (m, 3H), 7.40-7.31 (m, 2H), 7.28 (s, 1H), 6.94 (d, 1H), 4.95 (s, 2H), 3.87 (t, 2H), 3.82 (s, 2H), 3.67-3.62 (m, 4H), 3.22-3.14 (m, 1H), 3.14-3.06 (m, 2H), 3.00 (t, 4H), 2.09 (s, 3H), 1.41 (s, 2H), 1.37-1.20 (m, 4H), 1.20-0.95 (m, 6H), 0.85 (s, 6H). MS (ESI) m/e 820.2 (M+H)⁺.

### 1.45. Synthesis of 6-[5-(2-aminoethoxy)-8-(1,3-benzothiazol-2-ylcarbamoyl)-3,4-dihydroisoquinolin-2(1H)-yl]-3-{1-[(3,5-dimethyl-7-{2-[methyl(2-sulfoethyl)amino]ethoxy}tricyclo[3.3.1.1^{3,7}]dec-1-yl)methyl]-5-methyl-1H-pyrazol-4-yl}pyridine-2-carboxylic Acid (Compound W2.46)

### 1.45.1. 6-[8-(1,3-benzothiazol-2-ylcarbamoyl)-5-(2-{[(benzyloxy)carbonyl]amino}ethoxy)-3,4-dihydroisoquinolin-2(1H)-yl]-3-[1-({3,5-dimethyl-7-[(2,2,7,7,13-pentamethyl-10,10-dioxido-3,3-diphenyl-4,9-dioxa-10λ⁶-thia-13-aza-3-silapentadecan-15-yl)oxy]tricyclo[3.3.1.1^{3,7}]dec-1-yl}methyl)-5-methyl-1H-pyrazol-4-yl]pyridine-2-carboxylic Acid

The title compound was prepared as described in Example 1.2.8, replacing Example 1.2.7 with Example 1.35.

### 1.45.2. 6-[5-(2-aminoethoxy)-8-(1,3-benzothiazol-2-ylcarbamoyl)-3,4-dihydroisoquinolin-2(1H)-yl]-3-{1-[(3,5-dimethyl-7-{2-[methyl(2-sulfoethyl)amino]ethoxy}tricyclo[3.3.1.1^{3,7}]dec-1-yl)methyl]-5-methyl-1H-pyrazol-4-yl}pyridine-2-carboxylic Acid

The title compound was prepared as described in Example 1.34.4, replacing Example 1.34.3 with Example 1.46.1. ¹H NMR (500 MHz, dimethyl sulfoxide-d₆) δ ppm 12.74 (s, 2H), 8.96 (s, 1H), 8.03 (d, 1H), 7.94 (s, 3H), 7.72-7.81 (m, 2H), 7.53 (d, 1H), 7.47 (t, 1H), 7.35 (t, 1H), 7.28 (s, 1H), 7.02 (t, 2H), 5.03 (s, 2H), 4.26 (t, 2H), 3.92 (t, 2H), 3.83 (s, 2H), 3.23-3.38 (m, 4H), 3.13-3.25 (m, 1H), 2.82-3.00 (m, 4H), 2.78 (d, 3H), 2.11 (s, 3H), 1.23-1.50 (m, 6H), 0.95-1.21 (m, 6H), 0.86 (s, 6H). MS (ESI) m/e 927.2 (M+H)⁺.

### 1.46. Synthesis of 6-[8-(1,3-benzothiazol-2-ylcarbamoyl)-5-{2-[(2-sulfoethyl)amino]ethoxy}-3,4-dihydroisoquinolin-2(1H)-yl]-3-{1-[(3,5-dimethyl-7-{2-[methyl(2-sulfoethyl)amino]ethoxy}tricyclo[3.3.1.1^{3,7}]dec-1-yl)methyl]-5-methyl-1H-pyrazol-4-yl}pyridine-2-carboxylic Acid (Compound W2.47)

### 1.46.1. 6-[8-(1,3-benzothiazol-2-ylcarbamoyl)-5-[(2,2,7,7-tetramethyl-10,10-dioxido-3,3-diphenyl-4,9-dioxa-10λ⁶-thia-13-aza-3-silapentadecan-15-yl)oxy]-3,4-dihydroisoquinolin-2(1H)-yl]-3-{1-[(3,5-dimethyl-7-{2-[methyl(2-sulfoethyl)amino]ethoxy}tricyclo[3.3.1.1^{3,7}]dec-1-yl)methyl]-5-methyl-1H-pyrazol-4-yl}pyridine-2-carboxylic Acid

The title compound was prepared as described in Example 1.2.8, replacing Example 1.2.7 with Example 1.46.2.

### 1.46.2. 6-[8-(1,3-benzothiazol-2-ylcarbamoyl)-5-{2-[(2-sulfoethyl)amino]ethoxy}-3,4-dihydroisoquinolin-2(1H)-yl]-3-{1-[(3,5-dimethyl-7-{2-[methyl(2-sulfoethyl)amino]ethoxy}tricyclo[3.3.1.1^{3,7}]dec-1-yl)methyl]-5-methyl-1H-pyrazol-4-yl}pyridine-2-carboxylic Acid

Example 1.47.1 (100 mg) in dichloromethane (5 mL) was treated with trifluoroacetic acid (5 mL) overnight. The reaction mixture was concentrated and purified by reverse phase chromatography (C18 column), eluting with 20-60% acetonitrile in water containing 0.1% v/v trifluoroacetic acid, to provide the title compound. ¹H NMR (400 MHz, dimethyl sulfoxide-d₆) δ ppm m 12.74 (s, 1H), 8.96 (d, 1H), 8.64 (s, 2H), 8.02 (d, 1H), 7.76 (dd, 2H), 7.41-7.57 (m, 2H), 7.24-7.40 (m, 2H), 7.02 (t, 2H), 5.03 (s, 2H), 4.23-4.42 (m, 2H), 3.90 (t, 2H), 3.83 (s, 2H), 3.25-3.40 (m, 6H), 3.12-3.24 (m, 2H), 2.81-3.01 (m, 6H), 2.78 (d, 3H), 2.10 (s, 3H), 1.22-1.47 (m, 6H), 0.97-1.21 (m, 6H), 0.86 (s, 6H). MS (ESI) m/e 1035.3 (M+H)⁺.

### 1.47. Synthesis of 6-[8-(1,3-benzothiazol-2-ylcarbamoyl)-3,4-dihydroisoquinolin-2(1H)-yl]-3-{1-[(3,5-dimethyl-7-{2-[(2-sulfoethyl){2-[(2-sulfoethyl)amino]ethyl}amino]ethoxy}tricyclo[3.3.1.1^{3,7}]dec-1-yl)methyl]-5-methyl-1H-pyrazol-4-yl}pyridine-2-carboxylic Acid (Compound W2.48)

### 1.47.1. 6-[8-(1,3-benzothiazol-2-ylcarbamoyl)-3,4-dihydroisoquinolin-2(1H)-yl]-3-{1-[(3,5-dimethyl-7-([2,2,7,7-tetramethyl-10,10-dioxido-3,3-diphenyl-16-(2-sulfoethyl)-4,9-dioxa-10λ⁶-thia-13,16-diaza-3-silaoctadecan-18-yl]oxy)tricyclo[3.3.1.1^{3,7}]dec-1-yl)methyl]-5-methyl-1H-pyrazol-4-yl}pyridine-2-carboxylic Acid

The title compound was prepared as described in Example 1.2.8, replacing Example 1.2.7 with Example 1.33.2.

### 1.47.2. 6-[8-(1,3-benzothiazol-2-ylcarbamoyl)-3,4-dihydroisoquinolin-2(1H)-yl]-3-{1-[(3,5-dimethyl-7-(2-[(2-sulfoethyl){2-[(2-sulfoethyl)amino]ethyl)amino]ethoxy}tricyclo[3.3.1.1^{3,7}]dec-1-yl)methyl]-5-methyl-1H-pyrazol-4-yl}pyridine-2-carboxylic Acid

The title compound was prepared as described in Example 1.47.2, replacing Example 1.47.1 with Example 1.48.1. ¹H NMR (400 MHz, dimethyl sulfoxide-d₆) δ ppm 12.87 (s, 3H), 8.55 (s, 4H), 8.04 (d, 2H), 7.79 (d, 2H), 7.62 (d, 1H), 7.40-7.56 (m, 3H), 7.32-7.40 (m, 2H), 7.29 (s, 1H), 6.96 (d, 2H), 4.96 (s, 3H), 3.89 (t, 2H), 3.83 (s, 2H), 3.47 (d, 2H), 3.36 (s, 2H), 3.18-3.30 (m, 2H), 3.01 (t, 2H), 2.94 (t, 2H), 2.82 (t, 2H), 2.11 (s, 3H), 1.26-1.49 (m, 6H), 0.96-1.20 (m, 6H), 0.87 (s, 6H). MS (ESI) m/e 1005.2 (M+H)⁺.

### 1.48. Synthesis of 6-[8-(1,3-benzothiazol-2-ylcarbamoyl)-5-{2-[(2-carboxyethyl)amino]ethoxy}-3,4-dihydroisoquinolin-2(1H)-yl]-3-{1-[(3,5-dimethyl-7-{2-[methyl(2-sulfoethyl)amino]ethoxy}tricyclo[3.3.1.1^{3,7}]dec-1-yl)methyl]-5-methyl-1H-pyrazol-4-yl}pyridine-2-carboxylic Acid (Compound W2.49)

### 1.48.1. 6-(8-(benzo[d]thiazol-2-ylcarbamoyl)-5-(2-((3-(tert-butoxy)-3-oxopropyl)amino)ethoxy)-3,4-dihydroisoquinolin-2(1H)-yl)-3-(1-((3,5-dimethyl-7-(2-(methyl(2-sulfoethyl)amino)ethoxy)adamantan-1-yl)methyl)-5-methyl-1H-pyrazol-4-yl)picolinic Acid

The title compound was prepared as described in Example 1.32.3, replacing Example 1.32.2 with Example 1.46.2.

### 1.48.2. −[8-(1,3-benzothiazol-2-ylcarbamoyl)-5-{2-[(2-carboxyethyl)amino]ethoxy}-3,4-dihydroisoquinolin-2(1H)-yl]-3-{1-[(3,5-dimethyl-7-{2-[methyl(2-sulfoethyl)amino]ethoxy}tricyclo[3.3.1.1^{3,7}]dec-1-yl)methyl]-5-methyl-1H-pyrazol-4-yl}pyridine-2-carboxylic Acid

The title compound was prepared as described in Example 1.6.2, replacing Example 1.6.1 with Example 1.49.1. ¹H NMR (400 MHz, dimethyl sulfoxide-d₆) δ ppm 12.75 (s, 1H), 8.96 (s, 1H), 8.59 (s, 2H), 8.03 (d, 1H), 7.72-7.82 (m, 2H), 7.54 (d, 1H), 7.43-7.51 (m, 2H), 7.35 (t, 1H), 7.28 (s, 1H), 7.02 (dd, 2H), 5.02 (s, 2H), 4.34 (s, 2H), 3.93 (s, 2H), 3.83 (s, 2H), 3.62 (s, 2H), 2.84-3.01 (m, 4H), 2.78 (d, 3H), 2.65-2.75 (m, 2H), 2.11 (s, 3H), 1.20-1.45 (m, 7H), 0.95-1.21 (m, 6H), 0.86 (s, 6H). MS (ESI) m/e 999.2 (M+H)⁺.

### 1.49. Synthesis of 3-{1-[(3,5-dimethyl-7-{2-[(3-phosphonopropyl)(piperidin-4-yl)amino]ethoxy}tricyclo[3.3.1.1^{3,7}]dec-1-yl)methyl]-5-methyl-1H-pyrazol-4-yl}-6-[8-([1,3]thiazolo[4,5-b]pyridin-2-ylcarbamoyl)-3,4-dihydroisoquinolin-2(1H)-yl]pyridine-2-carboxylic Acid (Compound W2.50)

### 1.49.1. tert-butyl 3-(1-((3-(2-((1-(tert-butoxycarbonyl)piperidin-4-yl)amino)ethoxy)-5,7-dimethyladamantan-1-yl)methyl)-5-methyl-1H-pyrazol-4-yl)-6-(8-(thiazolo[4,5-b]pyridin-2-ylcarbamoyl)-3,4-dihydroisoquinolin-2(1H)-yl)picolinate

Example 1.23.2 (205 mg) was dissolved in dichloromethane (2.4 mL), and tert-butyl 4-oxopiperidine-1-carboxylate (51 mg) and sodium triacetoxyborohydride (75 mg) were added. The reaction was stirred at room temperature for two hours. More dichloromethane was added, and the reaction was poured into to saturated aqueous NaHCO₃ solution. The organic layer was washed with brine and dried over sodium sulfate. After filtration and concentration, the reside was purified by silica gel chromatography on a Grace Reveleris Amino cartridge, eluting with a gradient of 0.5 to 5.0% methanol in dichloromethane, to give the title compound. MS (ESI) m/e 986.3 (M+H)⁺.

### 1.49.2. 3-{1-[(3,5-dimethyl-7-{2-[(3-phosphonopropyl)(piperidin-4-yl)amino]ethoxy}tricyclo[3.3.1.1^{3,7}]dec-1-yl)methyl]-5-methyl-1H-pyrazol-4-yl}-6-[8-([1,3]thiazolo[4,5-b]pyridin-2-ylcarbamoyl)-3,4-dihydroisoquinolin-2(1H)-yl]pyridine-2-carboxylic Acid

Example 1.50.1 (94 mg) was dissolved in dichloromethane (1 mL), then Example 1.14.2 (25 mg) and sodium triacetoxyborohydride (30 mg) were added. The reaction was stirred at room temperature for four hours. Trifluoroacetic acid (1.5 mL) was added, and the reaction stirred at room temperature overnight. The reaction mixture was concentrated and purified by reverse phase chromatography (C18 column), eluting with 10-90% acetonitrile in water containing 0.1% v/v trifluoroacetic acid, to provide the title compound as a trifluoroacetic acid salt. ¹H NMR (400 MHz, dimethyl sulfoxide-d₆) δ ppm 8.82 (br s, 1H) 8.60 (dd, 1H), 8.52 (dd, 1H), 8.50 (br s, 1H), 7.66 (d, 1H), 7.50 (d, 1H), 7.46 (d, 1H), 7.38 (m, 2H), 7.30 (s, 1H), 6.97 (d, 1H), 4.98 (s, 2H), 3.89 (t, 2H), 3.83 (s, 2H) 3.69 (m, 2H), 3.61 (m, 1H), 3.44 (m, 2H) 3.23 (m, 4H), 3.02 (t, 2H), 2.93 (m, 2H), 2.18 (m, 2H), 2.10 (s, 3H), 1.92 (m, 2H), 1.83 (m, 2H), 1.64 (m, 2H), 1.44 (s, 2H), 1.31 (m, 4H), 1.14 (m, 4H), 1.04 (m, 2H), 0.87 (s, 6H). MS (ESI) m/e 952.3 (M+H)⁺.

### 1.50. Synthesis of 6-[4-(1,3-benzothiazol-2-ylcarbamoyl)-3,4-dihydro-2H-1,4-benzoxazin-6-yl]-3-{1-[(3,5-dimethyl-7-{2-[(2-sulfoethyl)amino]ethoxy}tricyclo[3.3.1.1^{3,7}]dec-1-yl)methyl]-5-methyl-1H-pyrazol-4-yl}pyridine-2-carboxylic Acid (Compound W2.51)

### 1.50.1. tert-butyl 3-(1-((3-(2-((tert-butyldimethylsilyl)oxy)ethoxy)-5,7-dimethyladamantan-1-yl)methyl)-5-methyl-1H-pyrazol-4-yl)-6-chloropicolinate

To a solution of Example 1.20.2 (3.2 g) in N,N-dimethylformamide (20 mL) was added imidazole (0.616 g) and chloro t-butyldimethylsilane (1.37 g). The mixture was stirred overnight. The reaction mixture was diluted with ethyl acetate (300 mL), washed with water and brine, and dried over sodium sulfate. Filtration and evaporation of the solvent gave the crude product that was purified by silica gel chromatography, eluting with 20% ethyl acetate in heptane, to provide the title compound. MS (ESI) m/e 645.4 (M+H)⁺.

### 1.50.2. tert-butyl 3-(1-((3-(2-((tert-butyldimethylsilyl)oxy)ethoxy)-5,7-dimethyladamantan-1-yl)methyl)-5-methyl-1H-pyrazol-4-yl)-6-(3,4-dihydro-2H-benzo[b][1,4]oxazin-6-yl)picolinate

To a solution of 6-(4,4,5,5-tetramethyl-1,3,2-dioxaborolan-2-yl)-3,4-dihydro-2H-benzo[b][1,4]oxazine (507 mg) in 1,4-dioxane (10 mL) and water (5 mL) was added Example 1.51.1 (1.25 g), bis(triphenylphosphine)palladium(II)dichloride (136 mg), and cesium fluoride (884 mg). The mixture was stirred at 120° C. under microwave conditions (Biotage, Initiator) for 20 minutes. The mixture was diluted with ethyl acetate (500 mL), washed with water and brine, and dried over sodium sulfate. Filtration and evaporation of the solvent gave a residue that was purified by silica gel chromatography, eluting with 20% ethyl acetate in heptane followed by 5% methanol in dichloromethane, to provide the title compound. MS (ESI) m/e 744.1 (M+H)⁺.

### 1.50.3. tert-butyl 6-(4-(benzo[d]thiazol-2-ylcarbamoyl)-3,4-dihydro-2H-benzo[b][1,4]oxazin-6-yl)-3-(1-((3-(2-((tert-butyldimethylsilyl)oxy)ethoxy)-5,7-dimethyladamantan-1-yl)methyl)-5-methyl-1H-pyrazol-4-yl)picolinate

To an ambient suspension of bis(2,5-dioxopyrrolidin-1-yl) carbonate (295 mg) in acetonitrile (10 mL) was added benzo[d]thiazol-2-amine (173 mg), and the mixture was stirred for 1 hour. A solution of Example 1.51.2 (710 mg) in acetonitrile (10 mL) was added, and the suspension was vigorously stirred overnight. The mixture was diluted with ethyl acetate (300 mL), washed with water and brine, and dried over sodium sulfate. Filtration and evaporation of the solvent gave a residue that was purified by silica gel chromatography, eluting with 20% ethyl acetate in heptane, to give the title compound. MS (ESI) m/e 920.2 (M+H)⁺.

### 1.50.4. tert-butyl 6-(4-(benzo[d]thiazol-2-ylcarbamoyl)-3,4-dihydro-2H-benzo[b][1,4]oxazin-6-yl)-3-(1-((3-(2-hydroxyethoxy)-5,7-dimethyladamantan-1-yl)methyl)-5-methyl-1H-pyrazol-4-yl)picolinate

To a solution of Example 1.51.3 (1.4 g) in tetrahydrofuran (10 mL) was added tetrabutyl ammonium fluoride (1.0M in tetrahydrofuran, 6 mL). The mixture was stirred for 3 hours. The mixture was diluted with ethyl acetate (300 mL), washed with water and brine, and dried over sodium sulfate. Filtration and evaporation of the solvent gave title product, which was used in the next reaction without further purification. MS (ESI) m/e 806.0 (M+H)⁺.

### 1.50.5. tert-butyl 6-(4-(benzo[d]thiazol-2-ylcarbamoyl)-3,4-dihydro-2H-benzo[b][1,4]oxazin-6-yl)-3-(1-((3,5-dimethyl-7-(2-((methylsulfonyl)oxy)ethoxy)adamantan-1-yl)methyl)-5-methyl-1H-pyrazol-4-yl)picolinate

To a cooled (0° C.) solution of Example 1.51.4 (1.2 g) in dichloromethane (20 mL) and triethylamine (2 mL) was added methanesulfonyl chloride (300 mg). The mixture was stirred for 4 hours. The reaction mixture was diluted with ethyl acetate (200 mL), washed with water and brine, and dried over sodium sulfate. Filtration and evaporation of the solvent gave title product, which was used in the next reaction without further purification. MS (ESI) m/e 884.1 (M+H)⁺.

### 1.50.6. tert-butyl 3-(1-((3-(2-azidoethoxy)-5,7-dimethyladamantan-1-yl)methyl)-5-methyl-1H-pyrazol-4-yl)-6-(4-(benzo[d]thiazol-2-ylcarbamoyl)-3,4-dihydro-2H-benzo[b][1,4]oxazin-6-yl)picolinate

To a solution of Example 1.51.5 (1.5 g) in N,N-dimethylformamide (20 mL) was added sodium azide (331 mg). The mixture was stirred for 48 hours. The reaction mixture was diluted with ethyl acetate (200 mL), washed with water and brine, and dried over sodium sulfate. Filtration and evaporation of the solvent gave a residue that was purified by silica gel chromatography, eluting with 20% ethyl acetate in dichloromethane, to provide the title compound. MS (ESI) m/e 831.1 (M+H)⁺.

### 1.50.7. tert-butyl 3-(1-((3-(2-aminoethoxy)-5,7-dimethyladamantan-1-yl)methyl)-5-methyl-1H-pyrazol-4-yl)-6-(4-(benzo[d]thiazol-2-ylcarbamoyl)-3,4-dihydro-2H-benzo[b][1,4]oxazin-6-yl)picolinate

To a solution of Example 1.51.6 (1.5 g) in tetrahydrofuran (30 mL) was added Pd/C (10%, 200 mg). The mixture was stirred under 1 atmosphere of hydrogen overnight. The reaction mixture was filtered, and the filtrate was concentrated under vacuum to give crude product. MS (ESI) m/e 805.1 (M+H)⁺.

### 1.50.8. 66-[4-(1,3-benzothiazol-2-ylcarbamoyl)-3,4-dihydro-2H-1,4-benzoxazin-6-yl]-3-{1-[(3,5-dimethyl-7-{2-[(2-sulfoethyl)amino]ethoxy}tricyclo[3.3.1.1^{3,7}]dec-1-yl)methyl]-5-methyl-1H-pyrazol-4-yl}pyridine-2-carboxylic Acid

To a solution of Example 1.51.7 (164 mg) in N,N-dimethylformamide (10 mL) and N,N-diisopropylethylamine (0.5 mL) was added 4-((tert-butyldiphenylsilyl)oxy)-2,2-dimethylbutyl ethenesulfonate (91 mg). The mixture was stirred overnight. The reaction mixture was diluted with ethyl acetate (200 mL), washed with water and brine, and dried over sodium sulfate. Filtration and evaporation of the solvent gave a residue that was dissolved in tetrahydrofuran (2 mL). Tetrabutyl ammonium fluoride (1 mL, 1M in tetrahydrofuran) was added, and the mixture was stirred overnight. The mixture was concentrated under vacuum, and the residue was dissolved in dichloromethane/trifluoroacetic acid (1:1, 6 mL), which was allowed to sit overnight. After evaporation of the solvent, the residue was purified by reverse phase HPLC (Gilson system), eluting with 10-85% acetonitrile in water containing 0.1% v/v trifluoroacetic acid, to provide the title compound. ¹H NMR (400 MHz, dimethyl sulfoxide-d₆) δ ppm 8.74 (s, 1H), 8.35 (s, 2H), 7.94-8.00 (m, 1H), 7.86 (s, 1H), 7.71-7.82 (m, 2H), 7.46 (s, 1H), 7.34-7.44 (m, 2H), 7.24 (t, 1H), 7.02 (d, 1H), 4.28-4.39 (m, 2H), 4.10-4.19 (m, 2H), 3.90 (s, 3H), 3.55-3.61 (m, 4H), 3.21-3.30 (m, 3H), 3.07-3.16 (m, 3H), 2.23 (s, 3H), 1.44 (s, 2H), 0.98-1.37 (m, 9H), 0.89 (s, 6H). MS (ESI) m/e 856.1 (M+H)⁺.

### 1.51. Synthesis of 6-[8-(1,3-benzothiazol-2-ylcarbamoyl)-5-(3-sulfopropoxy)-3,4-dihydroisoquinolin-2(1H)-yl]-3-[1-({3,5-dimethyl-7-[2-(methylamino)ethoxy]tricyclo[3.3.1.1^{3,7}]dec-1-yl}methyl)-5-methyl-1H-pyrazol-4-yl]pyridine-2-carboxylic Acid (Compound W2.52)

### 1.51.1. Methyl 2-(6-(tert-butoxycarbonyl)-5-(1-((3-(2-((tert-butoxycarbonyl)(methyl)amino)ethoxy)-5,7-dimethyladamantan-1-yl)methyl)-5-methyl-1H-pyrazol-4-yl)pyridin-2-yl)-5-(3-((2,2,2-trifluoro-1-(p-tolyl)ethoxy)sulfonyl)propoxy)-1,2,3,4-tetrahydroisoquinoline-8-carboxylate

To a solution of Example 1.31.8 (460 mg) in N,N-dimethylformamide (10 mL) was added 2,2,2-trifluoro-1-(p-tolyl)ethyl 3-iodopropane-1-sulfonate (239 mg, prepared according to J. Org. Chem., 2013, 78, 711-716) and K₂CO₃ (234 mg), and the mixture was stirred overnight. The mixture was diluted with ethyl acetate (200 mL), washed with water and brine, and dried over sodium sulfate. Filtration and evaporation of the solvent gave a residue that was purified by silica gel chromatography, eluting with 20% ethyl acetate in heptane, to provide the title compound. MS (ESI) m/e 1018.5 (M+H)⁺.

### 1.51.2. 2-(6-(tert-butoxycarbonyl)-5-(1-((3-(2-((tert-butoxycarbonyl)(methyl)amino)ethoxy)-5,7-dimethyladamantan-1-yl)methyl)-5-methyl-1H-pyrazol-4-yl)pyridin-2-yl)-5-(3-((2,2,2-trifluoro-1-(p-tolyl)ethoxy)sulfonyl)propoxy)-1,2,3,4-tetrahydroisoquinoline-8-carboxylic Acid

To a solution of Example 1.52.1 (176 mg) in tetrahydrofuran (4 mL), methanol (3 mL) and water (3 mL) was added lithium hydroxide monohydrate (60 mg), and the mixture was stirred overnight. The mixture was then diluted with ethyl acetate (200 mL), washed with 1N aqueous HCl, water and brine, and dried over sodium sulfate. Filtration and evaporation of the solvent gave the title product, which was used in the next reaction without further purification. MS (ESI) m/e 1095.2 (M+H)⁺.

### 1.51.3. tert-butyl 6-(8-(benzo[d]thiazol-2-ylcarbamoyl)-5-(3-((2,2,2-trifluoro-1-(p-tolyl)ethoxy)sulfonyl)propoxy)-3,4-dihydroisoquinolin-2(1H)-yl)-3-(1-((−(2-((tert-butoxycarbonyl)(methyl)amino)ethoxy)-5,7-dimethyladamantan-1-yl)methyl)-5-methyl-1H-pyrazol-4-yl)picolinate

To a solution of Example 1.52.2 (117 mg) in dichloromethane (6 mL) was added benzo[d]thiazol-2-amine (19.27 mg), 1-ethyl-3-[3-(dimethylamino)propyl]-carbodiimide hydrochloride (37 mg) and 4-(dimethylamino)pyridine (23.5 mg), and the mixture was stirred overnight. The reaction mixture was diluted with ethyl acetate (200 mL), washed with water and brine, and dried over sodium sulfate. Filtration and evaporation of the solvent gave the title product. MS (ESI) m/e 1226.1 (M+H)⁺.

### 1.51.4. 6-[8-(1,3-benzothiazol-2-ylcarbamoyl)-5-(3-sulfopropoxy)-3,4-dihydroisoquinolin-2(1H)-yl]-3-[1-({3,5-dimethyl-7-[2-(methylamino)ethoxy]tricyclo[3.3.1.1^{3,7}]dec-1-yl}methyl)-5-methyl-1H-pyrazol-4-yl]pyridine-2-carboxylic Acid

Example 1.52.3 (130 mg) was dissolved in dichloromethane/trifluoroacetic acid (1:1, 6 mL) and stirred overnight. After evaporation of the solvent, the residue was dissolved in N,N-dimethylformamide/water (1:1, 12 mL) and purified by reverse phase HPLC (Gilson), eluting with 10 to 85% acetonitrile in water containing 0.1% v/v trifluoroacetic acid, to give the title compound. ¹H NMR (400 MHz, dimethyl sulfoxide-d₆) δ ppm 12.68 (s, 1H), 8.13-8.32 (m, 2H), 8.01 (d, 1H), 7.75 (dd, 2H), 7.42-7.56 (m, 2H), 7.29 (s, 1H), 7.28-7.34 (m, 1H), 7.00 (dd, 2H), 5.03 (s, 2H), 4.19 (t, 2H), 3.83 (s, 3H), 3.50-3.57 (m, 4H), 2.95-3.05 (m, 2H), 2.81 (t, 2H), 2.52-2.65 (m, 4H), 1.39 (s, 2H), 0.96-1.32 (m, 12H), 0.87 (s, 6H). MS (ESI) m/e 898.3 (M+H)⁺.

### 1.52. Synthesis of 3-{1-[(3,5-dimethyl-7-{2-[(2-sulfoethyl)amino]ethoxy}tricyclo[3.3.1.1^{3,7}]dec-1-yl)methyl]-5-methyl-1H-pyrazol-4-yl}-6-[1-([1,3]thiazolo[4,5-b]pyridin-2-ylcarbamoyl)-1,2,3,4-tetrahydroquinolin-7-yl]pyridine-2-carboxylic Acid (Compound W2.53)

### 1.52.1. tert-butyl 6-chloro-3-(1-((3-(2-hydroxyethoxy)-5,7-dimethyladamantan-1-yl)methyl)-5-methyl-1H-pyrazol-4-yl)picolinate

The title compound was prepared as described in Example 1.51.4, replacing Example 1.51.3 with Example 1.51.1.

### 1.52.2. tert-butyl 6-chloro-3-(1-((3,5-dimethyl-7-(2-((methylsulfonyl)oxy)ethoxy)adamantan-1-yl)methyl)-5-methyl-1H-pyrazol-4-yl)picolinate

To a cooled (0° C.) solution of Example 1.53.1 (1.89 g) in dichloromethane (30 mL) and triethylamine (3 mL) was added methanesulfonyl chloride (1.03 g), and the mixture was stirred for 4 hours. The reaction mixture was diluted with ethyl acetate (200 mL), washed with water and brine, and dried over sodium sulfate. Filtration and evaporation of the solvent gave the title product, which was used in the next reaction without further purification.

### 1.52.3. tert-butyl 3-(1-((3-(2-aminoethoxy)-5,7-dimethyladamantan-1-yl)methyl)-5-methyl-1H-pyrazol-4-yl)-6-chloropicolinate

Example 1.53.2 (2.2 g) was dissolved in 7N ammonia in methanol (40 mL), and the mixture was stirred at 80° C. under microwave conditions (Biotage Initiator) for 2 hours. The mixture was concentrated under vacuum and, and the residue was dissolved in ethyl acetate, washed with water and brine, and dried over sodium sulfate. Filtration and evaporation of the solvent provided the title compound.

### 1.52.4. tert-butyl 6-chloro-3-[1-({3,5-dimethyl-7-[(2,2,7,7-tetramethyl-10,10-dioxido-3,3-diphenyl-4,9-dioxa-10λ⁶-thia-13-aza-3-silapentadecan-15-yl)oxy]tricyclo[3.3.1.1^{3,7}]dec-1-yl}methyl)-5-methyl-1H-pyrazol-4-yl]pyridine-2-carboxylate

To a solution of Example 1.53.3 (1.59 g) in N,N-dimethylformamide (30 mL) was added 4-((tert-butyldiphenylsilyl)oxy)-2,2-dimethylbutyl ethenesulfonate (1.6 g) and N,N-diisopropylethylamine (1 mL), and the mixture was stirred for 4 days. The reaction mixture was dissolved in ethyl acetate (400 mL), washed with water and brine, and dried over sodium sulfate. Filtration and evaporation of the solvent gave the title product, which was used in the next reaction without further purification. MS (ESI) m/e 976.8 (M+H)⁺.

### 1.52.5. tert-butyl 3-{1-[(3-{[13-(tert-butoxycarbonyl)-2,2,7,7-tetramethyl-10,10-dioxido-3,3-diphenyl-4,9-dioxa-10λ⁶-thia-13-aza-3-silapentadecan-15-yl]oxy}-5,7-dimethyltricyclo[3.3.1.1^{3,7}]dec-1-yl)methyl]-5-methyl-1H-pyrazol-4-yl}-6-chloropyridine-2-carboxylate

To a solution of Example 1.53.4 (2.93 g) in tetrahydrofuran (50 mL) was added di-t-butyldicarbonate (0.786 g) and 4-(dimethylamino)pyridine (100 mg), and the mixture was stirred overnight. The mixture was concentrated under vacuum, and the residue was dissolved in ethyl acetate (300 mL), washed with 1N aqueous HCl solution, water and brine, and dried over sodium sulfate. Filtration and evaporation of the solvent gave a residue that was purified by silica gel chromatography, eluting with 20% ethyl acetate in heptane, to provide the title compound. MS (ESI) m/e 1076.9 (M+H)⁺.

### 1.52.6. tert-butyl 3-{1-[(3-{[13-(tert-butoxycarbonyl)-2,2,7,7-tetramethyl-10,10-dioxido-3,3-diphenyl-4,9-dioxa-10λ⁶-thia-13-aza-3-silapentadecan-15-yl]oxy}-5,7-dimethyltricyclo[3.3.1.1^{3,7}]dec-1-yl)methyl]-5-methyl-1H-pyrazol-4-yl}-6-(1,2,3,4-tetrahydroquinolin-7-yl)pyridine-2-carboxylate

To a solution of 7-(4,4,5,5-tetramethyl-1,3,2-dioxaborolan-2-yl)-1,2,3,4-tetrahydroquinoline (65 mg) in 1,4-dioxane (10 mL) and water (5 mL) was added Example 1.53.5 (220 mg), bis(triphenylphosphine)palladium(II)dichloride (7 mg), and cesium fluoride (45.6 mg). The mixture was stirred at 120° C. for 30 minutes under microwave conditions (Biotage Initiator). The mixture was diluted with ethyl acetate (200 mL), washed with water and brine, and dried over sodium sulfate. Filtration and evaporation of the solvent gave a residue that was purified by silica gel chromatography, eluting with 20% ethyl acetate in heptane, to give the title compound. MS (ESI) m/e 1173.9 (M+H)⁺.

### 1.52.7. 3-{1-[(3,5-dimethyl-7-{2-[(2-sulfoethyl)amino]ethoxy}tricyclo[3.3.1.1^{3,7}]dec-1-yl)methyl]-5-methyl-1H-pyrazol-4-yl}-6-[1-([1,3]thiazolo[4,5-b]pyridin-2-ylcarbamoyl)-1,2,3,4-tetrahydroquinolin-7-yl]pyridine-2-carboxylic Acid

To an ambient suspension of bis(2,5-dioxopyrrolidin-1-yl) carbonate (48.2 mg) in acetonitrile (10 mL) was added thiazolo[4,5-b]pyridin-2-amine (34 mg), and the mixture was stirred for 1 hour. A solution of Example 1.53.6 (220 mg) in acetonitrile (5 mL) was added, and the suspension was vigorously stirred overnight. The mixture was diluted with ethyl acetate (200 mL), washed with water and brine, and dried over sodium sulfate. Filtration and evaporation of the solvent gave a residue, which was dissolved in trifluoroacetic acid (10 mL) and stirred overnight. After evaporation of the solvent, the residue was purified by reverse phase HPLC (Gilson system), eluting with 10-85% acetonitrile in water containing 0.1% v/v trifluoroacetic acid, to provide the title compound. ¹H NMR (500 MHz, dimethyl sulfoxide-d₆) δ ppm 8.42-8.48 (m, 1H), 8.31-8.40 (m, 4H), 8.03 (d, 1H), 7.89 (d, 1H), 7.80 (d, 1H), 7.47 (s, 1H), 7.26-7.37 (m, 2H), 3.93-4.02 (m, 3H), 3.90 (s, 3H), 3.52-3.60 (m, 3H), 3.17-3.26 (m, 2H), 3.05-3.14 (m, 2H), 2.76-2.89 (m, 5H), 2.23 (s, 3H), 1.90-2.01 (m, 2H), 1.44 (s, 2H), 1.27-1.37 (m, 4H), 0.99-1.22 (m, 5H), 0.88 (s, 6H). MS (ESI) m/e 855.1 (M+H)⁺.

### 1.53. Synthesis of 3-{1-[(3,5-dimethyl-7-{2-[(2-sulfoethyl)amino]ethoxy}tricyclo[3.3.1.1^{3,7}]dec-1-yl)methyl]-5-methyl-1H-pyrazol-4-yl}-6-[8-([1,3]thiazolo[4,5-b]pyridin-2-ylcarbamoyl)naphthalen-2-yl]pyridine-2-carboxylic Acid (Compound W2.54)

### 1.53.1. tert-butyl 3-{1-[(3-{[13-(tert-butoxycarbonyl)-2,2,7,7-tetramethyl-10,10-dioxido-3,3-diphenyl-4,9-dioxa-10π⁶-thia-13-aza-3-silapentadecan-15-yl]oxy}-5,7-dimethyltricyclo[3.3.1.1^{3,7}]dec-1-yl)methyl]-5-methyl-1H-pyrazol-4-yl}-6-[8-(methoxycarbonyl)naphthalen-2-yl]pyridine-2-carboxylate

The title compound was prepared by substituting methyl 7-(4,4,5,5-tetramethyl-1,3,2-dioxaborolan-2-yl)-1-naphthoate for 7-(4,4,5,5-tetramethyl-1,3,2-dioxaborolan-2-yl)-1,2,3,4-tetrahydroquinoline in Example 1.53.6. MS (ESI) m/e 1226.6 (M+H)⁺.

### 1.53.2. 7-[6-(tert-butoxycarbonyl)-5-{1-[(3-{[13-(tert-butoxycarbonyl)-2,2,7,7-tetramethyl-10,10-dioxido-3,3-diphenyl-4,9-dioxa-10λ⁶-thia-13-aza-3-silapentadecan-15-yl]oxy}-5,7-dimethyltricyclo[3.3.1.1^{3,7}]dec-1-yl)methyl]-5-methyl-1H-pyrazol-4-yl}pyridin-2-yl]naphthalene-1-carboxylic Acid

To a solution of Example 1.54.1 (79 mg) in tetrahydrofuran (4 mL), methanol (3 mL) and water (3 mL) was added lithium hydroxide monohydrate (60 mg), and the mixture was stirred overnight. The reaction was diluted with ethyl acetate (200 mL), washed with 1N aqueous HCl, water and brine, and dried over sodium sulfate. Filtration and evaporation of the solvent gave the title product, which was used in the next step without further purification. MS (ESI) m/e 1211.6 (M+H)⁺.

### 1.53.3. 3-{1-[(3,5-dimethyl-7-{2-[(2-sulfoethyl)amino]ethoxy}tricyclo[3.3.1.1^{3,7}]dec-1-yl)methyl]-5-methyl-1H-pyrazol-4-yl}-6-[8-([1,3]thiazolo[4,5-b]pyridin-2-ylcarbamoyl)naphthalen-2-yl]pyridine-2-carboxylic Acid

To a solution of Example 1.54.2 (60 mg) in dichloromethane (4 mL) was added thiazolo[4,5-b]pyridin-2-amine (7.56 mg), 1-ethyl-3-[3-(dimethylamino)propyl]-carbodiimide hydrochloride (19 mg) and 4-(dimethylamino)pyridine (12.2 mg), and the mixture was stirred overnight. The reaction mixture was diluted with ethyl acetate (200 mL), washed with water and brine, and dried over sodium sulfate. Filtration and evaporation of the solvent gave the title product, which was dissolved in dichloromethane/trifluoroacetic acid (1:1, 6 mL) and stirred overnight. After evaporation of solvent, the residue was dissolved in N,N-dimethylformamide/water (1:1, 12 mL) and purified by reverse phase HPLC (Gilson system), eluting with 10-85% acetonitrile in water containing 0.1% trifluoroacetic acid, to give the title compound. ¹H NMR (400 MHz, dimethyl sulfoxide-d₆) δ ppm 13.42 (s, 1H), 9.05 (s, 1H), 8.51-8.69 (m, 2H), 8.31-8.41 (m, 2H), 8.18-8.26 (m, 4H), 8.06 (d, 1H), 7.97 (d, 1H), 7.68-7.79 (m, 1H), 7.49 (s, 1H), 7.40 (dd, 1H), 3.90 (s, 3H), 3.18-3.29 (m, 3H), 3.07-3.15 (m, 2H), 2.82 (t, 3H), 2.24 (s, 3H), 1.44 (s, 2H), 0.97-1.37 (m, 10H), 0.88 (s, 6H). MS (ESI) m/e 850.1 (M+H)⁺.

### 1.54. Synthesis of (1ξ)-1-({2-[5-(1-{[3-(2-aminoethoxy)-5,7-dimethyltricyclo[3.3.1.1^{3,7}]dec-1-yl]methyl}-5-methyl-1H-pyrazol-4-yl)-6-carboxypyridin-2-yl]-8-(1,3-benzothiazol-2-ylcarbamoyl)-1,2,3,4-tetrahydroisoquinolin-5-yl}methyl)-1,5-anhydro-D-glucitol (Compound W2.55)

### 1.54.1. (2R,3R,4S,5R)-3,4,5-tris(methoxymethoxy)-2-((methoxymethoxy)methyl)-6-methylenetetrahydro-2H-pyran

The title compound was prepared according to J. R. Walker et al., Bioorg. Med. Chem. 2006, 14, 3038-3048. MS (ESI) m/e 370 (M+NH₄)⁺.

### 1.54.2. 4-Bromo-3-cyanomethyl-benzoic Acid Methyl Ester

To a solution of trimethylsilanecarbonitrile (3.59 mL) in tetrahydrofuran (6 mL) was added 1M tetrabutylammonium fluoride (26.8 mL, 1 M in tetrahydrofuran) dropwise over 30 minutes. The solution was stirred at room temperature for 30 minutes. Methyl 4-bromo-3-(bromomethyl)benzoate (7.50 g) was dissolved in acetonitrile (30 mL) and was added to the first solution dropwise over 30 minutes. The solution was heated to 80° C. for 30 minutes and cooled. The solution was concentrated under reduced pressure, and the residue was purified by silica gel chromatography, eluting with 20-30% ethyl acetate in heptanes, to provide the title compound.

### 1.54.3. 3-(2-Aminoethyl)-4-bromobenzoic Acid Methyl Ester

Example 1.55.2 (5.69 g) was dissolved in tetrahydrofuran (135 mL), and 1 M borane (in tetrahydrofuran, 24.6 mL) was added. The solution was stirred at room temperature for 16 hours and was slowly quenched with methanol and 1 M aqueous hydrochloric acid. 4 M Aqueous hydrochloric acid (150 mL) was added, and the solution was stirred at room temperature for 16 hours. The mixture was concentrated under reduced pressure, and the pH was adjusted to between 11 and 12 using solid potassium carbonate. The solution was then extracted with dichloromethane (3×100 mL). The organic extracts were combined and dried over anhydrous sodium sulfate. The solution was filtered and concentrated under reduced pressure, and the residue was purified by silica gel chromatography, eluting with 10-20% methanol in dichloromethane, to provide the title compound. MS (ESI) m/e 258, 260 (M+H)⁺.

### 1.54.4. 4-Bromo-3-[2-(2,2,2-trifluoroacetylamino)-ethyl]-benzoic Acid Methyl Ester

Example 1.55.2 (3.21 g) was dissolved in dichloromethane (60 mL). The solution was cooled to 0° C., and triethylamine (2.1 mL) was added. Trifluoroacetic anhydride (2.6 mL) was added dropwise. The solution was stirred at 0° C. for ten minutes, and the cooling bath was removed. After 1 hour, water (50 mL) was added, and the solution was diluted with ethyl acetate (100 mL). 1 M Aqueous hydrochloric acid was added (50 mL), and the organic layer was separated, washed with 1 M aqueous hydrochloric acid, and washed with brine. The solution was dried with anhydrous sodium sulfate, filtered and concentrated under reduced pressure to provide the title compound. MS (ESI) m/e 371, 373 (M+H)⁺.

### 1.54.5. 5-Bromo-2-(2,2,2-trifluoroacetyl)-1,2,3,4-tetrahydroisoquinoline-8-carboxylic Acid Methyl Ester

Example 1.55.4 (4.40 g) and paraformaldehyde (1.865 g) were placed in a flask and concentrated sulfuric acid (32 mL) was added. The solution was stirred at room temperature for one hour. Cold water (120 mL) was added, and the solution was extracted with ethyl acetate (3×100 mL). The extracts were combined, washed with saturated aqueous sodium bicarbonate (100 mL) and water (100 mL), and dried over anhydrous sodium sulfate. The mixture was filtered and concentrated under reduced pressure. The residue was purified by silica gel chromatography, eluting with 20-30% ethyl acetate in heptanes, to provide the title compound. MS (ESI) m/e 366, 368 (M+H)⁺.

### 1.54.6. Methyl 2-(2,2,2-trifluoroacetyl)-5-(((3S,4R,5R,6R)-3,4,5-tris(methoxymethoxy)-6-((methoxymethoxy)methyl)tetrahydro-2H-pyran-2-yl)methyl)-1,2,3,4-tetrahydroisoquinoline-8-carboxylate

Example 1.55.1 (242 mg) was dissolved in tetrahydrofuran (7 mL) and 9-borabicyclo[3.3.1]nonane (3.0 mL) was added dropwise. The solution was refluxed for 4.5 hours and allowed to cool to room temperature. Potassium phosphate (3M, 0.6 mL) was added, and the solution was stirred for 10 minutes. The solution was then degassed and flushed with nitrogen three times. Separately, Example 1.55.5 (239 mg) and dichloro[1,1′-bis(diphenylphosphino)ferrocene]palladium (II) dichloromethane adduct (39 mg) were dissolved in N,N-dimethylformamide (7 mL), and the solution was degassed and flushed with nitrogen three times. The N,N-dimethylformamide solution was added dropwise to the tetrahydrofuran solution, and the mixture was stirred for 18 hours. HCl solution (0.1 M aqueous, 25 mL) was added, and the solution was extracted with ethyl acetate (30 mL) three times. The organic extracts were combined, washed with brine, dried over anhydrous sodium sulfate, filtered and concentrated. The residue was purified by silica gel chromatography, eluting with 30-50% ethyl acetate in heptanes, to yield the title compound. MS (ESI) m/e 710 (M+NH₄)⁺.

### 1.54.7. Methyl 5-(((3S,4R,5R,6R)-3,4,5-tris(methoxymethoxy)-6-((methoxymethoxy)methyl)tetrahydro-2H-pyran-2-yl)methyl)-1,2,3,4-tetrahydroisoquinoline-8-carboxylate

Example 1.55.6 (247 mg) was dissolved in methanol (1 mL), tetrahydrofuran (1 mL), and water (0.5 mL). Potassium carbonate (59 mg) was added, and the solution was stirred at room temperature for 16 hours. The solution was diluted with ethyl acetate (10 mL) and washed with saturated aqueous sodium bicarbonate (1 mL). The organic layer was dried over anhydrous sodium sulfate, filtered and concentrated under reduced pressure to yield the title compound. MS (ESI) m/e 600 (M+H)⁺.

### 1.54.8. Methyl 2-(5-bromo-6-(tert-butoxycarbonyl)pyridin-2-yl)-5-(((3S,4R,5R,6R)-3,4,5-tris(methoxymethoxy)-6-((methoxymethoxy)methyl)tetrahydro-2H-pyran-2-yl)methyl)-1,2,3,4-tetrahydroisoquinoline-8-carboxylate

The title compound was prepared by substituting Example 1.55.7 for methyl 1,2,3,4-tetrahydroisoquinoline-8-carboxylate in Example 1.1.11. MS (ESI) m/e 799, 801 (M-tert-butyl)⁺.

### 1.54.9. Methyl 2-(6-(tert-butoxycarbonyl)-5-(4,4,5,5-tetramethyl-1,3,2-dioxaborolan-2-yl)pyridin-2-yl)-5-(((3S,4R,5R,6R)-3,4,5-tris(methoxymethoxy)-6-((methoxymethoxy)methyl)tetrahydro-2H-pyran-2-yl)methyl)-1,2,3,4-tetrahydroisoquinoline-8-carboxylate

The title compound was prepared by substituting Example 1.55.8 for Example 1.1.11 in Example 1.2.1. MS (ESI) m/e 903 (M+H)⁺, 933 (M+MeOH−H)⁻.

### 1.54.10.2-((3-((4-iodo-5-methyl-1H-pyrazol-1-yl)methyl)-5,7-dimethyladamantan-1-yl)oxy)ethanamine

The title compound was prepared by substituting Example 1.13.1 for Example 1.10.4 in Example 1.10.5. MS (ESI) m/e 444 (M+H)⁺.

### 1.54.11. tert-butyl (2-((3-((4-iodo-5-methyl-1H-pyrazol-1-yl)methyl)-5,7-dimethyladamantan-1-yl)oxy)ethyl)carbamate

The title compound was prepared by substituting Example 1.55.10 for Example 1.10.5 in Example 1.10.6. MS (ESI) m/e 544 (M+H)⁺, 488 (M-tert-butyl)⁺, 542 (M−H)⁻.

### 1.54.12.Methyl 2-(6-(tert-butoxycarbonyl)-5-(1-((3-(2-((tert-butoxycarbonyl)amino)ethoxy)-5,7-dimethyladamantan-1-yl)methyl)-5-methyl-1H-pyrazol-4-yl)pyridin-2-yl)-5-(((3R,4S,5S,6S)-3,4,5-tris(methoxymethoxy)-6-((methoxymethoxy)methyl)tetrahydro-2H-pyran-2-yl)methyl)-1,2,3,4-tetrahydroisoquinoline-8-carboxylate

The title compound was prepared by substituting Example 1.55.9 for Example 1.2.1 and Example 1.55.11 for Example 1.13.3 in Example 1.13.4. MS (ESI) m/e 1192 (M+H)⁺.

### 1.54.13.2-(6-(tert-butoxycarbonyl)-5-(1-((3-(2-((tert-butoxycarbonyl)amino)ethoxy)-5,7-dimethyladamantan-1-yl)methyl)-5-methyl-1H-pyrazol-4-yl)pyridin-2-yl)-5-(((3R,4S,5S,6S)-3,4,5-tris(methoxymethoxy)-6-((methoxymethoxy)methyl)tetrahydro-2H-pyran-2-yl)methyl)-1,2,3,4-tetrahydroisoquinoline-8-carboxylic Acid

The title compound was prepared by substituting Example 1.55.12 for Example 1.2.4 in Example 1.2.5. MS (ESI) m/e 1178 (M+H)⁺, 1176 (M−H)⁻.

### 1.54.14.Tert-butyl 6-(8-(benzo[d]thiazol-2-ylcarbamoyl)-5-(((3R,4S,5S,6S)-3,4,5-tris(methoxymethoxy)-6-((methoxymethoxy)methyl)tetrahydro-2H-pyran-2-yl)methyl)-3,4-dihydroisoquinolin-2(1H)-yl)-3-(1-((3-(2-((tert-butoxycarbonyl)amino)ethoxy)-5,7-dimethyladamantan-1-yl)methyl)-5-methyl-1H-pyrazol-4-yl)picolinate

The title compound was prepared by substituting Example 1.55.13 for Example 1.52.2 in Example 1.52.3. MS (ESI) m/e 1310 (M+H)⁺, 1308 (M−H)⁻.

### 1.54.15. (1ξ)-1-({2-[5-(1-{[3-(2-aminoethoxy)-5,7-dimethyltricyclo[3.3.1.1]^{3,7}]dec-1-yl]methyl}-5-methyl-1H-pyrazol-4-yl)-6-carboxypyridin-2-yl]-8-(1,3-benzothiazol-2-ylcarbamoyl)-1,2,3,4-tetrahydroisoquinolin-5-yl}methyl)-1,5-anhydro-D-glucitol

The title compound was prepared by substituting Example 1.55.14 for Example 1.52.3 and 4M aqueous hydrochloric acid for trifluoroacetic acid in Example 1.52.4. ¹H NMR (400 MHz, dimethyl sulfoxide-d₆) δ ppm 7.96 (d, 1H), 7.73 (d, 1H), 7.58 (bs, 3H), 7.46 (d, 1H), 7.43-7.39 (m, 2H), 7.30 (d, 1H), 7.27-7.25 (m, 2H), 6.88 (d, 1H), 4.90 (q, 2H), 3.76 (m, 4H), 3.51 (m, 1H), 3.21 (d, 2H), 3.18 (d, 1H), 3.12 (m, 2H), 3.02 (m, 4H), 2.93 (m, 4H), 2.83 (m, 2H), 2.59 (m, 2H), 2.03 (s, 3H), 1.44 (s, 1H), 1.34 (s, 2H), 1.23 (q, 4H), 1.07 (m, 4H), 0.97 (q, 2H), 0.80 (s, 6H). MS (ESI) m/e 922 (M+H)⁺, 920 (M−H)⁻.

### 1.55. Synthesis of 6-[8-(1,3-benzothiazol-2-ylcarbamoyl)-3,4-dihydroisoquinolin-2(1H)-yl]-3-{1-[(3-{2-[(3-carboxypropyl)amino]ethoxy}-5,7-dimethyltricyclo[3.3.1.1^{3,7}]dec-1-yl)methyl]-5-methyl-1H-pyrazol-4-yl}pyridine-2-carboxylic Acid (Compound W2.56)

### 1.55.1. tert-butyl 6-(8-(benzo[d]thiazol-2-ylcarbamoyl)-3,4-dihydroisoquinolin-2(1H)-yl)-3-(1-((3-(2-((4-(tert-butoxy)-4-oxobutyl)amino)ethoxy)-5,7-dimethyladamantan-1-yl)methyl)-5-methyl-1H-pyrazol-4-yl)picolinate

To a solution of Example 1.2.7 (0.103 g) and tert-butyl 4-bromobutanoate (0.032 g) in dichloromethane (0.5 mL) was added N,N-diisopropylethylamine (0.034 mL) at 50° C. in a sealed amber vial overnight. The reaction was concentrated, dissolved in dimethyl sulfoxide/methanol (1:1, 2 mL) and purified by reverse phase HPLC using a Gilson system, eluting with 5-75% acetonitrile in water containing 0.1% v/v trifluoroacetic acid. The desired fractions were combined and freeze-dried to provide the title compound. MS (ESI) m/e 944.6 (M+1).

### 1.55.2. 6-[8-(1,3-benzothiazol-2-ylcarbamoyl)-3,4-dihydroisoquinolin-2(1H)-yl]-3-{1-[(3-{2-[(3-carboxypropyl)amino]ethoxy}-5,7-dimethyltricyclo[3.3.1.1^{3,7}]dec-1-yl)methyl]-5-methyl-1H-pyrazol-4-yl}pyridine-2-carboxylic Acid

A solution of Example 1.56.1 (0.049 g) was dissolved in dichloromethane (1 mL) and treated with trifluoroacetic acid (0.5 mL) and the mixture was stirred overnight. The reaction was concentrated, dissolved in a (1:1) N,N-dimethylformamide/water mixture (2 mL), and purified by reverse phase HPLC using a Gilson system, eluting with 5-75% acetonitrile in water containing 0.1% v/v trifluoroacetic acid. The desired fractions were combined and freeze-dried to provide the title compound. ¹H NMR (400 MHz, dimethyl sulfoxide-d₆) δ ppm 13.09-12.32 (m, 2H), 8.31 (s, 2H), 8.03 (d, 1H), 7.79 (d, 1H), 7.62 (d, 1H), 7.54-7.40 (m, 3H), 7.40-7.32 (m, 2H), 7.29 (s, 1H), 6.96 (d, 1H), 4.96 (s, 2H), 3.89 (t, 2H), 3.83 (s, 2H), 3.55 (d, 2H), 3.02 (q, 4H), 2.92 (q, 2H), 2.33 (t, 2H), 2.10 (s, 3H), 1.80 (p, 2H), 1.43 (s, 2H), 1.30 (q, 4H), 1.21-0.95 (m, 6H), 0.87 (s, 6H). MS (ESI) m/e 832.3 (M+H)⁺.

### 1.56. Synthesis of 6-[8-(1,3-benzothiazol-2-ylcarbamoyl)naphthalen-2-yl]-3-{1-[(3,5-dimethyl-7-{2-[(3-phosphonopropyl)amino]ethoxy}tricyclo[3.3.1.1^{3,7}]dec-1-yl)methyl]-5-methyl-1H-pyrazol-4-yl}pyridine-2-carboxylic Acid (Compound W2.57)

### 1.56.1. tert-butyl 3-(1-((3-(2-hydroxyethoxy)-5,7-dimethyladamantan-1-yl)methyl)-5-methyl-1H-pyrazol-4-yl)-6-(8-(methoxycarbonyl)naphthalen-2-yl)picolinate

To a solution of methyl 7-(4,4,5,5-tetramethyl-1,3,2-dioxaborolan-2-yl)-1-naphthoate (2.47 g) in 1,4-dioxane (40 mL) and water (20 mL) was added Example 1.20.2 (4.2 g), bis(triphenylphosphine)palladium(II)dichloride (556 mg), and cesium fluoride (3.61 g). The mixture was refluxed overnight, diluted with ethyl acetate (400 mL) and washed with water and brine. The organic layer was dried over sodium sulfate, filtered, and concentrated. The residue was purified by silica gel chromatography, eluting with 20% ethyl acetate in dichloromethane and then with 5% methanol in dichloromethane, to provide the title compound. MS (ESI) m/e 680.84 (M+H)⁺.

### 1.56.2. tert-butyl 3-(1-((3,5-dimethyl-7-(2-((methylsulfonyl)oxy)ethoxy)adamantan-1-yl)methyl)-5-methyl-1H-pyrazol-4-yl)-6-(8-(methoxycarbonyl)naphthalen-2-yl)picolinate

To a cooled (0° C.) solution of Example 1.57.1 (725 mg) in dichloromethane (10 mL) and triethylamine (0.5 mL) was added methanesulfonyl chloride (0.249 mL). The mixture was stirred at room temperature for 4 hours, diluted with ethyl acetate, and washed with water and brine. The organic layer was dried over sodium sulfate, filtered, and concentrated to provide the title compound. MS (ESI) m/e 758.93 (M+H)⁺.

### 1.56.3. tert-butyl 3-(1-((3-(2-azidoethoxy)-5,7-dimethyladamantan-1-yl)methyl)-5-methyl-1H-pyrazol-4-yl)-6-(8-(methoxycarbonyl)naphthalen-2-yl)picolinate

To a solution of Example 1.57.2 (4.2 g) in N,N-dimethylformamide (30 mL) was added sodium azide (1.22 g). The mixture was stirred at room temperature for 96 hours, diluted with ethyl acetate (600 mL) and washed with water and brine. The organic layer was dried over sodium sulfate, filtered, and concentrated to provide the title compound. MS (ESI) m/e 704.86 (M+H)⁺.

### 1.56.4. 7-(5-(1-((3-(2-azidoethoxy)-5,7-dimethyladamantan-1-yl)methyl)-5-methyl-1H-pyrazol-4-yl)-6-(tert-butoxycarbonyl)pyridin-2-yl)-1-naphthoic Acid

To a solution of Example 1.57.3 (3.5 g) in tetrahydrofuran/methanol/H₂O (2:1:1, 30 mL) was added lithium hydroxide monohydrate (1.2 g), and the mixture was stirred at room temperature overnight. The reaction mixture was acidified with 1N aqueous HCl solution, diluted with ethyl acetate (600 mL) and washed with water and brine. The organic layer was dried over sodium sulfate, filtered, and concentrated to provide the title compound. MS (ESI) m/e 691.82 (M+H)⁺.

### 1.56.5. tert-butyl 3-(1-((3-(2-azidoethoxy)-5,7-dimethyladamantan-1-yl)methyl)-5-methyl-1H-pyrazol-4-yl)-6-(8-(benzo[d]thiazol-2-ylcarbamoyl)naphthalen-2-yl)picolinate

To a solution of Example 1.57.4 (870 mg) in N,N-dimethylformamide (10 mL) was added benzo[d]thiazol-2-amine (284 mg), fluoro-N,N,N′N′-tetramethylformamidium hexafluorophosphate (499 mg) and N,N-diisopropylethylamine (488 mg). The mixture was stirred at 60° C. for 3 hours, diluted with ethyl acetate (200 mL), and washed with water and brine. The organic layer was dried over sodium sulfate, filtered, and concentrated to provide the title compound. MS (ESI) m/e 824.02 (M+H)⁺.

### 1.56.6. tert-butyl 3-(1-((3-(2-aminoethoxy)-5,7-dimethyladamantan-1-yl)methyl)-5-methyl-1H-pyrazol-4-yl)-6-(8-(benzo[d]thiazol-2-ylcarbamoyl)naphthalen-2-yl)picolinate

To a solution of Example 1.57.5 (890 mg) in tetrahydrofuran (30 mL) was added Pd/C (90 mg, 5%). The mixture was stirred under a hydrogen atmosphere at room temperature overnight, and filtered. The filtrate was concentrated to provide the title compound. MS (ESI) m/e 798.2 (M+H)⁺.

### 1.56.7. 6-[8-(1,3-benzothiazol-2-ylcarbamoyl)naphthalen-2-yl]-3-{1-[(3,5-dimethyl-7-{2-[(3-phosphonopropyl)amino]ethoxy}tricyclo[3.3.1.1^{3,7}]dec-1-yl)methyl]-5-methyl-1H-pyrazol-4-yl}pyridine-2-carboxylic Acid

To a solution of Example 1.57.6 (137 mg) in dichloromethane (6 mL) was added Example 1.14.2 (43 mg). The mixture was stirred at room temperature for 1.5 hours, and a solution of NaBH₄ (26 mg) in methanol (2 mL) was added. The mixture was stirred at room temperature for 2 hours, diluted with ethyl acetate (200 mL) and washed with 2N aqueous NaOH solution, water and brine. The organic layer was dried over sodium sulfate, filtered, and concentrated. The residue was dissolved in dichloromethane (5 mL) and treated with trifluoroacetic acid (5 mL) overnight. The reaction mixture was concentrated. The residue was purified by reverse phase HPLC (Gilson system), eluting with a gradient of 10-85% acetonitrile in water containing 0.1% v/v trifluoroacetic acid solution, to provide the title compound. ¹H NMR (500 MHz, dimethyl sulfoxide-d₆) δ 9.03 (s, 1H), 8.48-8.35 (m, 3H), 8.29-8.16 (m, 3H), 8.08 (dd, 1H), 8.03 (dd, 1H), 7.94 (d, 1H), 7.82 (d, 1H), 7.71 (dd, 1H), 7.53-7.47 (m, 2H), 7.38 (td, 1H), 4.81-0.53 (m, 89H). MS (ESI) m/e 863.2 (M+H)⁺.

### 1.57. Synthesis of 6-[8-(1,3-benzothiazol-2-ylcarbamoyl)-3,4-dihydroisoquinolin-2(1H)-yl]-3-(1-{[3-(2-{[4-(beta-D-glucopyranosyloxy)benzyl]amino}ethoxy)-5,7-dimethyltricyclo[3.3.1.1^{3,7}]dec-1-yl]methyl}-5-methyl-1H-pyrazol-4-yl)pyridine-2-carboxylic Acid (Compound W2.58)

To a solution of Example 1.3.1 (44.5 mg) in tetrahydrofuran (2 mL) and acetic acid (0.2 mL) was added 4-(((2S,3R,4R,5S,6R)-3,4,5-trihydroxy-6-(hydroxymethyl)tetrahydro-2H-pyran-2-yl)oxy)benzaldehyde (17 mg) and MgSO₄ (300 mg). The mixture was stirred at room temperature for 1 hour before the addition of sodium cyanoborohydride on resin (300 mg). The mixture was stirred at room temperature overnight and filtered. The filtrate was concentrated, and the residue was purified by reverse phase HPLC (Gilson system), eluting with a gradient of 10-85% acetonitrile in water containing 0.1% v/v trifluoroacetic acid solution, to provide the title compound. MS (ESI) m/e 1015.20 (M+H)⁺.

### 1.58. Synthesis of 3-(1-{[3-(2-{[4-(beta-D-allopyranosyloxy)benzyl]amino}ethoxy)-5,7-dimethyltricyclo[3.3.1.1^{3,7}]dec-1-yl]methyl}-5-methyl-1H-pyrazol-4-yl)-6-[8-(1,3-benzothiazol-2-ylcarbamoyl)-3,4-dihydroisoquinolin-2(1H)-yl]pyridine-2-carboxylic Acid (Compound W2.59)

To a solution of Example 1.3.1 (44.5 mg) in tetrahydrofuran (2 mL) and acetic acid (0.2 mL) was added 4-(((2S,3R,4S,5S,6R)-3,4,5-trihydroxy-6-(hydroxymethyl)tetrahydro-2H-pyran-2-yl)oxy)benzaldehyde (17 mg) and MgSO₄ (300 mg), and the mixture was stirred at room temperature for 1 hour before the addition of sodium cyanoborohydride on resin (300 mg). The mixture was stirred at room temperature overnight and filtered. The filtrate was concentrated, and the residue was purified by reverse phase HPLC (Gilson system), eluting with a gradient of 10-85% acetonitrile in water containing 0.1% v/v trifluoroacetic acid, to provide the title compound. MS (ESI) m/e 1015.20 (M+H)⁺.

### 1.59. Synthesis of 3-{1-[(3-{2-[azetidin-3-yl(2-sulfoethyl)amino]ethoxy}-5,7-dimethyltricyclo[3.3.1.1^{3,7}]dec-1-yl)methyl]-5-methyl-1H-pyrazol-4-yl}-6-[8-(1,3-benzothiazol-2-ylcarbamoyl)-3,4-dihydroisoquinolin-2(1H)-yl]pyridine-2-carboxylic Acid (Compound W2.60)

### 1.59.1. tert-butyl 6-(8-(benzo[d]thiazol-2-ylcarbamoyl)-3,4-dihydroisoquinolin-2(1H)-yl)-3-(1-((3-(2-((1-(tert-butoxycarbonyl)azetidin-3-yl)(2-((4-(tert-butyldiphenylsilyl)hydroxy-2,2-dimethylbutoxy)sulfonyl)ethyl)amino)ethoxy)-5,7-dimethyladamantan-1-yl)methyl)-5-methyl-1H-pyrazol-4-yl)picolinate

A solution of Example 1.2.8 (0.075 g), tert-butyl 3-oxoazetidine-1-carboxylate (0.021 g) and sodium triacetoxyborohydride (0.025 g) in dichloromethane (0.5 mL) was stirred at room temperature overnight. The reaction was loaded onto silica gel and eluted with 0-10% methanol in dichloromethane to give the title compound. MS (ESI) m/e 1403.9 (M+1).

### 1.59.2. 3-{1-[(3-{2-[azetidin-3-yl(2-sulfoethyl)amino]ethoxy}-5,7-dimethyltricyclo[3.3.1.1^{3,7}]dec-1-yl)methyl]-5-methyl-1H-pyrazol-4-yl}-6-[8-(1,3-benzothiazol-2-ylcarbamoyl)-3,4-dihydroisoquinolin-2(1H)-yl]pyridine-2-carboxylic Acid

A solution of Example 1.60.1 (0.029 g) in dichloromethane (1 mL) was treated with trifluoroacetic acid (1 mL) and stirred overnight. The reaction was concentrated, dissolved in 1:1 dimethyl sulfoxide/methanol (2 mL), and the mixture was purified by reverse phase HPLC using a Gilson system, eluting with 10-80% acetonitrile in water containing 0.1% v/v trifluoroacetic acid. The desired fractions were combined and freeze-dried to provide the title compound. ¹H NMR (400 MHz, dimethyl sulfoxide-d₆) δ ppm 12.86 (s, 1H), 8.81 (s, 2H), 8.04 (d, 1H), 7.79 (d, 1H), 7.62 (d, 1H), 7.52 (d, 1H), 7.50-7.46 (m, 1H), 7.44 (d, 1H), 7.40-7.33 (m, 2H), 7.30 (s, 1H), 6.96 (d, 1H), 4.96 (s, 2H), 4.37 (q, 1H), 4.27 (s, 2H), 4.11 (s, 2H), 3.89 (t, 2H), 3.83 (s, 2H), 3.58-3.54 (m, 2H), 3.32 (t, 2H), 3.24 (s, 2H), 3.01 (t, 2H), 2.85 (t, 2H), 2.10 (s, 3H), 1.48-0.97 (m, 12H), 0.87 (s, 6H). MS (ESI) m/e 909.2 (M+H)⁺.

### 1.60. Synthesis of 3-{1-[(3-{2-[(3-aminopropyl)(2-sulfoethyl)amino]ethoxy}-5,7-dimethyltricyclo[3.3.1.1^{3,7}]dec-1-yl)methyl]-5-methyl-1H-pyrazol-4-yl}-6-[8-(1,3-benzothiazol-2-ylcarbamoyl)-3,4-dihydroisoquinolin-2(1H)-yl]pyridine-2-carboxylic Acid (Compound W2.61)

### 1.60.1. 6-(8-(benzo[d]thiazol-2-ylcarbamoyl)-3,4-dihydroisoquinolin-2(1H)-yl)-3-(1-((3-(2-((3-((tert-butoxycarbonyl)amino)propyl)(2-sulfoethyl)amino)ethoxy)-5,7-dimethyladamantan-1-yl)methyl)-5-methyl-1H-pyrazol-4-yl)picolinic Acid

The title compound was prepared using the procedure for Example 1.33.1, replacing tert-butyl (2-oxoethyl)carbamate with tert-butyl (3-oxopropyl)carbamate. MS (ESI) m/e 1011.5 (M+H).

### 1.60.2. 3-{1-[(3-{2-[(3-aminopropyl)(2-sulfoethyl)amino]ethoxy}-5,7-dimethyltricyclo[3.3.1.1^{3,7}]dec-1-yl)methyl]-5-methyl-1H-pyrazol-4-yl}-6-[8-(1,3-benzothiazol-2-ylcarbamoyl)-3,4-dihydroisoquinolin-2(1H)-yl]pyridine-2-carboxylic Acid

The title compound was prepared as described in Example 1.6.2, replacing Example 1.6.1 with Example 1.61.1. ¹H NMR (400 MHz, dimethyl sulfoxide-d₆) δ ppm 12.87 (s, 1H), 9.10 (s, 1H), 8.04 (d, 1H), 7.88-7.67 (m, 4H), 7.62 (d, 1H), 7.57-7.40 (m, 3H), 7.36 (td, 2H), 6.96 (d, 1H), 4.96 (s, 2H), 4.05-3.78 (m, 4H), 3.41-3.08 (m, 3H), 2.94 (tt, 6H), 2.11 (s, 3H), 1.92 (t, 2H), 1.53-0.95 (m, 11H), 0.87 (s, 6H). MS (ESI) m/e 911.3 (M+H).

### 1.61. Synthesis of 6-[1-(1,3-benzothiazol-2-ylcarbamoyl)-1,2,3,4-tetrahydroquinolin-7-yl]-3-{1-[(3-{2-[(2-carboxyethyl)amino]ethoxy}-5,7-dimethyltricyclo[3.3.1.1^{3,7}]dec-1-yl)methyl]-5-methyl-1H-pyrazol-4-yl}pyridine-2-carboxylic Acid (Compound W2.62)

### 1.61.1. tert-butyl 3-(1-((3-(2-((3-(tert-butoxy)-3-oxopropyl)amino)ethoxy)-5,7-dimethyladamantan-1-yl)methyl)-5-methyl-1H-pyrazol-4-yl)-6-chloropicolinate

To an ambient solution of Example 1.53.3 (521 mg) in ethanol (10 mL) was added triethylamine (3 mL) followed by tert-butyl acrylate (2 mL). The mixture was stirred at room temperature for 3 hours and then concentrated to dryness. The residue was dissolved in ethyl acetate (200 mL), and the solution was washed with water and brine. The organic layer was dried over sodium sulfate, filtered and concentrated under reduced pressure to give the title compound, which was used in the next reaction without further purification. MS (ESI) m/e 657.21 (M+H)⁺.

### 1.61.2. tert-butyl 3-(1-((3-(2-((3-(tert-butoxy)-3-oxopropyl)(tert-butoxycarbonyl)amino)ethoxy)-5,7-dimethyladamantan-1-yl)methyl)-5-methyl-1H-pyrazol-4-yl)-6-chloropicolinate

To a solution of Example 1.62.1 (780 mg) in tetrahydrofuran (10 mL) was added di-tert-butyl dicarbonate (259 mg) followed by a catalytic amount of 4-dimethylaminopyridine. The reaction was stirred at room temperature for 3 hours and then concentrated to dryness. The residue was dissolved in ethyl acetate (200 mL), and the solution was washed with saturated aqueous NaHCO₃ solution, water and brine. The organic layer was dried over sodium sulfate, filtered and concentrated under reduced pressure. The residue was purified by chromatography on silica gel, eluting with 20% ethyl acetate in heptane, to give the title compound. MS (ESI) m/e 757.13 (M+H)⁺.

### 1.61.3. tert-butyl 3-(1-((3-(2-((3-(tert-butoxy)-3-oxopropyl)(tert-butoxycarbonyl)amino)ethoxy)-5,7-dimethyladamantan-1-yl)methyl)-5-methyl-1H-pyrazol-4-yl)-6-(1,2,3,4-tetrahydroquinolin-7-yl)picolinate

To a solution of 7-(4,4,5,5-tetramethyl-1,3,2-dioxaborolan-2-yl)-1,2,3,4-tetrahydroquinoline (234 mg) in 1,4-dioxane (10 mL) and water (5 mL) was added Example 1.62.2 (685 mg), bis(triphenylphosphine)palladium(II)dichloride (63.2 mg), and cesium fluoride (410 mg). The mixture was heated to 120° C. for 30 minutes by microwave irradiation (Biotage Initiator). The reaction was quenched by the addition of ethyl acetate and water. The layers were separated, and the organic layer was washed with brine, dried over sodium sulfate, filtered and concentrated under reduced pressure. The residue was purified by chromatography on silica gel, eluting with 20% ethyl acetate in heptane, to give the title compound. MS (ESI) m/e 854.82 (M+H)⁺.

### 1.61.4. tert-butyl 6-(1-(benzo[d]thiazol-2-ylcarbamoyl)-1,2,3,4-tetrahydroquinolin-7-yl)-3-(1-((3-(2-((3-(tert-butoxy)-3-oxopropyl)(tert-butoxycarbonyl)amino)ethoxy)-5,7-dimethyladamantan-1-yl)methyl)-5-methyl-1H-pyrazol-4-yl)picolinate

To an ambient suspension of bis(2,5-dioxopyrrolidin-1-yl) carbonate (150 mg) in acetonitrile (10 mL) was added benzo[d]thiazol-2-amine (88 mg), and the mixture was stirred for 1 hour. A solution of Example 1.62.3 (500 mg) in acetonitrile (2 mL) was added, and the suspension was vigorously stirred overnight. The reaction was quenched by the addition of ethyl acetate and water. The layers were separated, and the organic layer was washed with brine, dried over sodium sulfate, filtered and concentrated under reduced pressure. The residue was purified by chromatography on silica gel, eluting with 20% ethyl acetate in dichloromethane, to give the title compound. MS (ESI) m/e 1030.5 (M+H)⁺.

### 1.61.5. 6-[1-(1,3-benzothiazol-2-ylcarbamoyl)-1,2,3,4-tetrahydroquinolin-7-yl]-3-{1-[(3-{2-[(2-carboxyethyl)amino]ethoxy}-5,7-dimethyltricyclo[3.3.1.1^{3,7}]dec-1-yl)methyl]-5-methyl-1H-pyrazol-4-yl}pyridine-2-carboxylic Acid

To an ambient solution of Example 1.62.4 (110 mg) in dichloromethane (0.53 mL) was added trifluoroacetic acid (0.53 mL). The reaction was stirred overnight and was concentrated to a viscous oil. The residue was dissolved in dimethyl sulfoxide/methanol (1:1, 2 mL) and purified by reverse phase HPLC (Gilson system), eluting with 10-55% acetonitrile in 0.1% trifluoroacetic acid in water, to give the title compound. ¹H NMR (400 MHz, dimethyl sulfoxide-d₆) δ ppm 13.10 (s, 3H), 8.37 (s, 1H), 8.26 (s, 2H), 7.98 (d, 1H), 7.86-7.71 (m, 3H), 7.44 (s, 1H), 7.39-7.31 (m, 1H), 7.26 (d, 1H), 7.19 (t, 1H), 3.92 (d, 2H), 3.87 (s, 2H), 3.55 (t, 2H), 3.17-3.00 (m, 4H), 2.80 (t, 2H), 2.62 (t, 2H), 2.19 (s, 3H), 1.95-1.88 (m, 2H), 1.43 (s, 2H), 1.33-1.25 (m, 4H), 1.18-1.11 (m, 4H), 1.09-0.97 (m, 2H), 0.85 (s, 6H). MS (ESI) m/e 818.0 (M+H)⁺.

### 1.63 Synthesis of 6-[8-(1,3-benzothiazol-2-ylcarbamoyl)-3,4-dihydroisoquinolin-2(1H)-yl]-3-{1-[(3-{2-[(N⁶,N⁶-dimethyl-L-lysyl)(methyl)amino]ethoxy}-5,7-dimethyltricyclo[3.3.1.1^{3,7}]dec-1-yl)methyl]-5-methyl-1H-pyrazol-4-yl}pyridine-2-carboxylic Acid

A solution of (S)-2-((((9H-fluoren-9-yl)methoxy)carbonyl)amino)-6-(dimethylamino)hexanoic acid (0.029 g) and 1-[bis(dimethylamino)methylene]-1H-1,2,3-triazolo[4,5-b]pyridinium 3-oxide hexafluorophosphate (0.028 g) was stirred together in N,N-dimethylformamide (0.5 mL) with N,N-diisopropylamine (0.035 mL). After stirring for 5 minutes, the solution was added to Example 1.13.7 (0.051 g) and stirring was continued at room temperature overnight. To the reaction was added diethylamine (0.070 mL), and the reaction was stirred for 2 hours. The reaction was diluted with N,N-dimethylformamide (1 mL), water (0.5 ml), and 2,2,2-trifluoroacetic acid (0.103 ml) then purified via reverse-phase HPLC using a gradient of 10% to 90% acetonitrile/water. The product containing fractions were collected and lyophilized to give the title compound. ¹H NMR (500 MHz, dimethyl sulfoxide-d₆) δ 9.59 (s, 1H), 8.41 (s, 1H), 8.12 (t, 3H), 8.01 (d, 1H), 7.85 (dd, 1H), 7.81 (d, 1H), 7.77 (dd, 1H), 7.47 (s, 1H), 7.38 (t, 1H), 7.30 (d, 1H), 7.22 (t, 1H), 3.97 (t, 2H), 3.89 (s, 2H), 3.49 (dt, 4H), 3.06 (s, 2H), 2.99 (q, 2H), 2.88 (s, 2H), 2.84 (t, 2H), 2.75 (d, 6H), 2.22 (s, 3H), 2.00-1.90 (m, 2H), 1.84-1.52 (m, 4H), 1.48-0.95 (m, 14H), 0.87 (d, 6H). MS (ESI) m/e 916.2 (M+H)⁺.

### 1.64 Synthesis of 3-{1-[(3-{2-[(3-aminopropyl)(methyl)amino]ethoxy}-5,7-dimethyltricyclo[3.3.1.1^{3,7}]dec-1-yl)methyl]-5-methyl-1H-pyrazol-4-yl}-6-[1-(1,3-benzothiazol-2-ylcarbamoyl)-1,2,3,4-tetrahydroquinolin-7-yl]pyridine-2-carboxylic Acid

### 1.64.1 6-(1-(benzo[d]thiazol-2-ylcarbamoyl)-1,2,3,4-tetrahydroquinolin-7-yl)-3-(1-((3-(2-((3-((tert-butoxycarbonyl)amino)propyl)methyl)amino)ethoxy)-5,7-dimethyladamantan-1-yl)methyl)-5-methyl-1H-pyrazol-4-yl)picolinic Acid

A solution of Example 1.21.5 (100 mg), N,N-diisopropylethylamine (68.9 μL) and tert-butyl (3-oxopropyl)carbamate (68.4 mg) in dichloromethane (3 mL) was stirred at ambient temperature for 2 hours, and NaCNBH₄ (8.27 mg) was added. The reaction was stirred at ambient temperature overnight. Methanol (1 mL) and water (0.2 mL) were added. The resulting mixture was stirred for 10 minutes and concentrated. The residue was dissolved in dimethyl sulfoxide and purified by reverse-phase HPLC on a Gilson system (C18 column), eluting with 30-80% acetonitrile in 0.1% trifluoroacetic acid water solution, to provide the title compound as a trifluoroacetic acid salt. MS (ESI) m/e 459.4 (M+2H)²⁺.

### 1.64.2 3-{1-[(3-{2-[(3-aminopropyl)(methyl)amino]ethoxy}-5,7-dimethyltricyclo[3.3.1.1^{3,7}]dec-1-yl)methyl]-5-methyl-1H-pyrazol-4-yl}-6-[1-(1,3-benzothiazol-2-ylcarbamoyl)-1,2,3,4-tetrahydroquinolin-7-yl]pyridine-2-carboxylic Acid

Example 1.64.1 (100 mg) in dichloromethane (4 mL) at 0° C. was treated with trifluoroacetic acid (1 mL) for 1 hour, and the mixture was concentrated. The residue was purified by reverse phase HPLC (C18 column), eluting with a gradient of 10-60% acetonitrile in 0.1% trifluoroacetic acid water solution, to provide the title compound as a trifluoroacetic acid salt. ¹H NMR (400 MHz, dimethyl sulfoxide-d₆) δ 9.38 (s, 1H), 8.37 (s, 1H), 7.98 (d, 1H), 7.90-7.69 (m, 6H), 7.44 (s, 2H), 7.35 (td, 1H), 7.27 (d, 1H), 7.22-7.16 (m, 1H), 3.94 (d, 2H), 3.87 (s, 2H), 3.64 (t, 2H), 3.28-2.98 (m, 4H), 2.87-2.70 (m, 8H), 2.19 (s, 3H), 1.90 (dp, 4H), 1.43 (s, 2H), 1.36-1.22 (m, 4H), 1.15 (s, 4H), 1.08-0.95 (m, 2H), 0.86 (s, 6H). MS (ESI) m/e 817.6 (M+H)⁺.

### 1.65 Synthesis of 3-{1-[(3-{2-[azetidin-3-yl(methyl)amino]ethoxy}-5,7-dimethyltricyclo[3.3.1.1^{3,7}]dec-1-yl)methyl]-5-methyl-1H-pyrazol-4-yl}-6-[1-(1,3-benzothiazol-2-ylcarbamoyl)-1,2,3,4-tetrahydroquinolin-7-yl]pyridine-2-carboxylic Acid

### 1.65.1 6-(1-(benzo[d]thiazol-2-ylcarbamoyl)-1,2,3,4-tetrahydroquinolin-7-yl)-3-(1-((3-(2-((1-(tert-butoxycarbonyl)azetidin-3-yl)(methyl)amino)ethoxy)-5,7-dimethyladamantan-1-yl)methyl)-5-methyl-1H-pyrazol-4-yl)picolinic Acid

The title compound was prepared using the procedure described in Example 1.64.1, substituting tert-butyl (3-oxopropyl)carbamate with tert-butyl 3-oxoazetidine-1-carboxylate. MS (ESI) m/e 915.3 (M+H)⁺.

### 1.65.2 3-{1-[(3-{2-[azetidin-3-yl(methyl)amino]ethoxy}-5,7-dimethyltricyclo[3.3.1.1^{3,7}]dec-1-yl)methyl]-5-methyl-1H-pyrazol-4-yl}-6-[1-(1,3-benzothiazol-2-ylcarbamoyl)-1,2,3,4-tetrahydroquinolin-7-yl]pyridine-2-carboxylic Acid

The title compound was prepared using the procedure in Example 1.64.2, substituting Example 1.64.1 with Example 1.65.1. ¹H NMR (400 MHz, dimethyl sulfoxide-d₆) δ 9.01 (s, 2H), 8.37 (s, 1H), 7.98 (d, 1H), 7.86-7.70 (m, 3H), 7.44 (s, 2H), 7.34 (td, 1H), 7.27 (d, 1H), 7.23-7.15 (m, 1H), 4.22 (s, 4H), 4.07 (s, 2H), 3.93 (t, 2H), 3.58 (t, 2H), 3.11 (s, 2H), 2.80 (t, 2H), 2.68 (s, 3H), 2.19 (s, 3H), 1.92 (p, 2H), 1.42 (s, 2H), 1.30 (s, 4H), 1.15 (s, 4H), 1.09-0.96 (m, 2H), 0.85 (s, 6H). MS (ESI) m/e 815.5 (M+H)⁺.

### 1.66 Synthesis of N⁶-(37-oxo-25,8,11,14,17,20,23,26,29,32,35-dodecaoxaheptatriacontan-37-yl)-L-lysyl-N-[2-({3-[(4-{6-[8-(1,3-benzothiazol-2-ylcarbamoyl)-3,4-dihydroisoquinolin-2(1H)-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl]-L-alaninamide

### 1.66.1 (S)-6-((((9H-fluoren-9-yl)methoxy)carbonyl)amino)-2-((tert-butoxycarbonyl)amino)hexanoic Acid

To a solution of (S)-6-amino-2-((tert-butoxycarbonyl)amino)hexanoic acid (8.5 g) in a mixture of 5% aqueous NaHCO₃ solution (300 mL) and dioxane (40 mL), chilled in an ice bath, was added dropwise a solution of (9H-fluoren-9-yl)methyl pyrrolidin-1-yl carbonate (11.7 g) in dioxane (40 mL). The reaction mixture was allowed to warm to room temperature and was stirred for 24 hours. Three additional vials were set up as described above. After the reaction was completed, all four reaction mixtures were combined, and the organic solvent was removed under vacuum. The aqueous residue was acidified to pH 3 with aqueous hydrochloric acid solution (1N) and then extracted with ethyl acetate (3×500 mL). The combined organic layers were washed with brine, dried over magnesium sulfate, filtered, and concentrated under vacuum to give a crude compound which was recrystallized from methyl tert-butyl ether to afford the title compound. ¹H NMR (400 MHz, chloroform-d) δ 11.05 (br. s., 1H), 7.76 (d, 2H), 7.59 (d, 2H), 7.45-7.27 (m, 4H), 6.52-6.17 (m, 1H), 5.16-4.87 (m, 1H), 4.54-4.17 (m, 4H), 3.26-2.98 (m, 2H), 1.76-1.64 (m, 1H), 1.62-1.31 (m, 14H).

### 1.66.2 tert-butyl 17-hydroxy-3,6,9,12,15-pentaoxaheptadecan-1-oate

To a solution of 3,6,9,12-tetraoxatetradecane-1,14-diol (40 g) in toluene (800 mL) was added portion-wise potassium tert-butoxide (20.7 g). The mixture was stirred at room temperature for 30 minutes. Tert-butyl 2-bromoacetate (36 g) was added dropwise to the mixture. The reaction was stirred at room temperature for 16 hours. Two additional vials were set up as described above. After the reactions were completed, all three reaction mixtures were combined. Water (500 mL) was added to the combined mixture, and the mixture was concentrated to 1 L. The mixture was extracted with dichloromethane and was washed with aqueous 1N potassium tert-butoxide solution (1 L). The organic layer was dried over Na₂SO₄, filtered and concentrated to obtain crude product, which was purified by silica gel column chromatography, eluting with dichloromethane:methanol 50:1, to obtain the title compound. ¹H NMR (400 MHz, chloroform-d) δ 4.01 (s, 2H), 3.75-3.58 (m, 21H), 1.46 (s, 9H).

### 1.66.3 tert-butyl 17-(tosyloxy)-3,6,9,12,15-pentaoxaheptadecan-1-oate

To a solution of Example 1.66.2 (30 g) in dichloromethane (500 mL) was added dropwise a solution of 4-methylbenzene-1-sulfonyl chloride (19.5 g) and triethylamine (10.3 g) in dichloromethane (500 mL) at 0° C. under a nitrogen atmosphere. The mixture was stirred at room temperature for 18 hours and was poured into water (100 mL). The solution was extracted with dichloromethane (3×150 mL), and the organic layer was washed with hydrochloric acid (6N, 15 mL) then NaHCO₃ (5% aqueous solution, 15 mL) followed by water (20 mL). The organic layer was dried over Na₂SO₄, filtered and concentrated to obtain a residue, which was purified by silica gel column chromatography, eluting with petroleum ether:ethyl acetate 10:1 to dichloromethane:methanol 5:1, to obtain the title compound. ¹H NMR (400 MHz, chloroform-d) δ 7.79 (d, 2H), 7.34 (d, 2H), 4.18-4.13 (m, 2H), 4.01 (s, 2H), 3.72-3.56 (m, 18H), 2.44 (s, 3H), 1.47 (s, 9H).

### 1.66.4 2,5,8,11,14,17,20,23,26,29,32,35-dodecaoxaheptatriacontan-37-oic Acid

To a solution of 2,5,8,11,14,17-hexaoxanonadecan-19-ol (32.8 g) in tetrahydrofuran (300 mL) was added sodium hydride (1.6 g) at 0° C. The mixture was stirred at room temperature for 4 hours. A solution of Example 1.66.3 (16 g) in tetrahydrofuran (300 mL) was added dropwise at room temperature to the reaction mixture. The resulting reaction mixture was stirred at room temperature for 16 hours and then water (20 mL) was added. The mixture was stirred at room temperature for another 3 hours to complete the tert-butyl ester hydrolysis. The final reaction mixture was concentrated under vacuum to remove the organic solvent. The aqueous residue was extracted with dichloromethane (2×150 mL). The aqueous layer was acidified to pH 3 and then extracted with ethyl acetate (2×150 mL). The aqueous layer was concentrated to obtain crude product, which was purified by silica gel column chromatography, eluting with a gradient of petroleum ether:ethyl acetate 1:1 to dichloromethane:methanol 5:1, to obtain the title compound. ¹H NMR (400 MHz, chloroform-d) δ 4.19 (s, 2H), 3.80-3.75 (m, 2H), 3.73-3.62 (m, 40H), 3.57 (dd, 2H), 3.40 (s, 3H).

### 1.66.5 (43S,46S)-43-((tert-butoxycarbonyl)amino)-46-methyl-37,44-dioxo-2,5,8,11,14,17,20,23,26,29,32,35-dodecaoxa-38,45-diazaheptatetracontan-47-oic Acid

Example 1.66.5 was synthesized using standard Fmoc solid phase peptide synthesis procedures and a 2-chlorotrityl resin. 2-Chlorotrityl resin (12 g, 100 mmol), (S)-2-((((9H-fluoren-9-yl)methoxy)carbonyl)amino)propanoic acid (10 g, 32.1 mmol) and N,N-diisopropylethylamine (44.9 mL, 257 mmol) in anhydrous, sieve-dried dichloromethane (100 mL) was shaken at 14° C. for 24 hours. The mixture was filtered and the cake was washed with dichloromethane (3×500 mL), dimethylformamide (2×250 mL) and methanol (2×250 mL) (for 5 minutes for each step). To the above resin was added 20% piperidine/dimethylformamide (100 mL) to remove the Fmoc group. The mixture was bubbled with nitrogen for 15 minutes and then filtered. The resin was washed with 20% piperidine/dimethylformamide (100 mL) another five times (5 minutes each step), and washed with dimethylformamide (5×100 mL) to give the deprotected, L-Ala loaded resin.

To a solution of Example 1.66.1 (9.0 g) in N,N-dimethylformamide (50 mL) was added hydroxybenzotriazole (3.5 g), 2-(6-chloro-1H-benzotriazole-1-yl)-1,1,3,3-tetramethylaminium hexafluorophosphate (9.3 g) and N,N-diisopropylethylamine (8.4 mL). The mixture was stirred at 20° C. for 30 minutes. The above mixture was added to the D-Ala loaded resin and mixed by bubbling with nitrogen at room temperature for 90 minutes. The mixture was filtered and the resin was washed with dimethylformamide (5 minutes each step). To the above resin was added approximately 20% piperidine/N,N-dimethylformamide (100 mL) to remove the Fmoc group. The mixture was bubbled with nitrogen for 15 minutes and filtered. The resin was washed with 20% piperidine/dimethylformamide (100 mL) for another five times (5 minutes for each step), and finally washed with dimethylformamide (5×100 mL).

To a solution of Example 1.66.4 (11.0 g) in N,N-dimethylformamide (50 mL) was added hydroxybenzotriazole (3.5 g), 2-(6-chloro-1H-benzotriazole-1-yl)-1,1,3,3-tetramethylaminium hexafluorophosphate (9.3 g) and N,N-diisopropylethylamine (8.4 mL), and the mixture was added to the resin and mixed by bubbling with nitrogen at room temperature for 3 hours. The mixture was filtered and the residue was washed with dimethylformamide (5×100 mL), dichloromethane (8×100 mL) (5 minutes for each step).

To the final resin was added 1% trifluoroacetic acid/dichloromethane (100 mL) and nitrogen was bubbled through for 5 minutes. The mixture was filtrated and the filtrate was collected. The cleavage operation was repeated for four times. The combined filtrate was brought to pH 7 by NaHCO₃ and washed with water. The organic layer was dried over Na₂SO₄, filtered and concentrated to obtain the title compound. ¹H NMR (400 MHz, methanol-d₄) δ 4.44-4.33 (m, 1H), 4.08-4.00 (m, 1H), 3.98 (s, 2H), 3.77-3.57 (m, 42H), 3.57-3.51 (m, 2H), 3.36 (s, 3H), 3.25 (t, 2H), 1.77 (br. s., 1H), 1.70-1.51 (m, 4H), 1.44 (s, 9H), 1.42-1.39 (m, 3H).

### 1.66.6 tert-butyl 6-(8-(benzo[d]thiazol-2-ylcarbamoyl)-3,4-dihydroisoquinolin-2(1H)-yl)-3-(1-((3-(((43S,46S)-43-((tert-butoxycarbonyl)amino)-46-methyl-37,44,47-trioxo-2,5,8,11,14,17,20,23,26,29,32,35-dodecaoxa-38,45,48-triazapentacontan-50-yl)oxy)-5,7-dimethyladamantan-1-yl)methyl)-5-methyl-1H-pyrazol-4-yl)picolinate

Example 1.66.5 (123 mg, 0.141 mmol), was mixed with 1-[bis(dimethylamino)methylene]-1H-1,2,3-triazolo[4,5-b]pyridinium 3-oxide hexafluorophosphate (58.9 mg) and N,N-diisopropylethylamine (0.049 mL) in N-methyl-2-pyrrolidone (1 mL) for 10 minutes and then added to a solution of Example 1.2.7 (142 mg) and N,N-diisopropylethylamine (0.049 mL) in N-methyl-2-pyrrolidone (1.5 mL). The reaction mixture was stirred at room temperature for two hours. The crude reaction mixture was purified by reverse phase HPLC using a Gilson system and a C18 25×100 mm column, eluting with 5-85% acetonitrile in water containing 0.1% v/v trifluoroacetic acid. The product fractions were lyophilized to give the title compound. MS (LC/MS) m/e 1695.5 (M+H)⁺.

### 1.66.7 3-(1-((3-(((43S,46S)-43-amino-46-methyl-37,44,47-trioxo-2,5,8,11,14,17,20,23,26,29,32,35-dodecaoxa-38,45,48-triazapentacontan-50-yl)oxy)-5,7-dimethyladamantan-1-yl)methyl)-5-methyl-1H-pyrazol-4-yl)-6-(8-(benzo[d]thiazol-2-ylcarbamoyl)-3,4-dihydroisoquinolin-2(1H)-yl)picolinic Acid

Example 1.66.6 (82 mg) was treated with 1 mL of trifluoroacetic acid at room temperature for 30 minutes. The solvent was evaporated under a gentle stream of nitrogen, and the residue was purified by reverse phase HPLC using a Gilson system and a C18 25×100 mm column, eluting with 5-85% acetonitrile in water containing 0.1% v/v trifluoroacetic acid. The product fractions were lyophilized to give the title compound as the trifluoroacetic acid salt. ¹H NMR (400 MHz, dimethyl sulfoxide-d₆) δ ppm 12.86 (s, 1H), 8.04 (dd, 4H), 7.64 (dt, 2H), 7.55-7.41 (m, 3H), 7.36 (q, 2H), 6.95 (d, 1H), 4.96 (s, 2H), 4.40-4.27 (m, 1H), 3.93-3.72 (m, 7H), 3.59-3.47 (m, 42H), 3.33-3.27 (m, 3H), 3.23 (s, 5H), 3.05 (dt, 5H), 2.10 (s, 3H), 1.72-1.64 (m, 2H), 1.48-1.36 (m, 4H), 1.35-1.16 (m, 10H), 1.16-0.94 (m, 6H), 0.84 (d, 6H). MS (ESI) m/e 751.8 (2M+H)²⁺.

### 1.67 Synthesis of methyl 6-[4-(3-{[2-({3-[(4-{6-[8-(1,3-benzothiazol-2-ylcarbamoyl)-3,4-dihydroisoquinolin-2(1H)-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl]amino}propyl)-1H-1,2,3-triazol-1-yl]-6-deoxy-beta-L-glucopyranoside

### 1.67.1 6-(8-(benzo[d]thiazol-2-ylcarbamoyl)-3,4-dihydroisoquinolin-2(1H)-yl)-3-(1-((3,5-dimethyl-7-(2-(pent-4-yn-1-ylamino)ethoxy)adamantan-1-yl)methyl)-5-methyl-1H-pyrazol-4-yl)picolinic Acid

To a solution of tert-butyl 3-(1-((3-(2-aminoethoxy)-5,7-dimethyladamantan-1-yl)methyl)-5-methyl-1H-pyrazol-4-yl)-6-(8-(benzo[d]thiazol-2-ylcarbamoyl)-3,4-dihydroisoquinolin-2(1H)-yl)picolinate (85 mg) in tetrahydrofuran (2 mL) was added pent-4-ynal (8.7 mg), acetic acid (20 mg, 0.318) and anhydrous sodium sulfate (300 mg). The mixture was stirred at room temperature for 1 hour. Sodium triacetoxyborohydride (45 mg) was added to the reaction mixture. The mixture was stirred at room temperature overnight. The reaction mixture was diluted with ethyl acetate (200 mL), washed with water and brine, and dried over anhydrous sodium sulfate. Filtration and evaporation of the solvent gave crude product, which was dissolved in dichloromethane (5 mL) and trifluoroacetic acid (3 mL). The mixture was stirred at room temperature overnight. After evaporation of the solvent, the residue was dissolved in dimethyl sulfoxide/methanol (1:1, 3 mL) and purified by reverse-phase HPLC on a Gilson system (C18 column), eluting with 20-80% acetonitrile in water containing 0.1% trifluoroacetic acid, to give the title compound. MS (APCI) m/e 812.2 (M+H)⁺.

### 1.67.2 methyl 6-[4-(3-{[2-({3-[(4-{6-[8-(1,3-benzothiazol-2-ylcarbamoyl)-3,4-dihydroisoquinolin-2(1H)-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl]amino}propyl)-1H-1,2,3-triazol-1-yl]-6-deoxy-beta-L-glucopyranoside

To a solution of (2R,3R,4S,5S,6S)-2-azido-6-(methoxycarbonyl)tetrahydro-2H-pyran-3,4,5-triyl triacetate (8.63 mg) in t-BuOH (2 mL) and water (1 mL) was added Example 1.67.1 (20 mg), copper(II) sulfate pentahydrate (2.0 mg) and sodium ascorbate (5 mg). The mixture was heated for 20 minutes at 100° C. under microwave conditions (Biotage Initiator). LiOH H₂O (50 mg) was added to the mixture, which was stirred at room temperature overnight. The mixture was neutralized with trifluoroacetic acid and purified by reverse-phase HPLC on a Gilson system (C18 column), eluting with 20-80% acetonitrile in water containing 0.1% trifluoroacetic acid, to give the title compound. MS (APCI) m/e 1032.2 (M+H)⁺.

### 1.68 Synthesis of 6-[8-(1,3-benzothiazol-2-ylcarbamoyl)naphthalen-2-yl]-3-{1-[(3-{2-[(2-carboxyethyl)amino]ethoxy}-5,7-dimethyltricyclo[3.3.1.1^{3,7}]dec-1-yl)methyl]-5-methyl-1H-pyrazol-4-yl}pyridine-2-carboxylic Acid

### 1.68.1 2-((3,5-dimethyl-7-((5-methyl-4-(4,4,5,5-tetramethyl-1,3,2-dioxaborolan-2-yl)-1H-pyrazol-1-yl)methyl)adamantan-1-yl)oxy)ethanol

To a solution of 2-((3-((4-iodo-5-methyl-1H-pyrazol-1-yl)methyl)-5,7-dimethyladamantan-1-yl)oxy)ethanol (8.9 g) and PdCl₂(dppf)-CH₂Cl₂ adduct (([1,1′-bis(diphenylphosphino)ferrocene]dichloropalladium(II) (1:1), 818 mg) in acetonitrile (120 mL) was added trimethylamine (10 mL) and 4,4,5,5-tetramethyl-1,3,2-dioxaborolane (12.8 mL). The mixture was stirred at reflux overnight. The mixture was cooled to room temperature and used in the next reaction without further work up. MS (ESI) m/e 467.3 (M+Na)⁺.

### 1.68.2 tert-butyl 6-chloro-3-(1-((3-(2-hydroxyethoxy)-5,7-dimethyladamantan-1-yl)methyl)-5-methyl-1H-pyrazol-4-yl)picolinate

To a solution of tert-butyl 3-bromo-6-chloropicolinate (6.52 g) in tetrahydrofuran (100 mL) and water (20 mL) was added Example 1.68.1 (9.90 g), (1S,3R,5R,7S)-1,3,5,7-tetramethyl-8-tetradecyl-2,4,6-trioxa-8-phosphaadamantane (0.732 g), tris(dibenzylideneacetone)dipalladium(0) (Pd₂(dba)₃, 1.02 g), and K₃PO₄ (23.64 g). The mixture was stirred at reflux overnight. The mixture was concentrated under reduced pressure, the residue was dissolved in ethyl acetate (500 mL), washed with water and brine, and dried over anhydrous sodium sulfate. Filtration and evaporation of the solvent gave crude product, which was purified by silica gel chromatography eluting with 20 to 40% ethyl acetate in dichloromethane to give the title compound. MS (ESI) m/e 530.3 (M+H)⁺.

### 1.68.3 tert-butyl 6-chloro-3-(1-((3,5-dimethyl-7-(2-((methylsulfonyl)oxy)ethoxy)adamantan-1-yl)methyl)-5-methyl-1H-pyrazol-4-yl)picolinate

To a cooled (0° C.) solution of Example 1.68.2 (3.88 g) in dichloromethane (30 mL) and triethylamine (6 mL) was added methanesulfonyl chloride (2.52 g). The mixture was stirred at room temperature for 4 hours. The reaction mixture was diluted with ethyl acetate (400 mL), washed with water and brine, and dried over anhydrous sodium sulfate. Filtration and evaporation of the solvent gave the crude product (4.6 g), which was used in the next reaction without further purification. MS (ESI) m/e 608.1 (M+H)⁺.

### 1.68.4 tert-butyl 3-{1-[(3-{2-[bis(tert-butoxycarbonyl)amino]ethoxy}-5,7-dimethyltricyclo[3.3.1.1^{3,7}]dec-1-yl)methyl]-5-methyl-1H-pyrazol-4-yl}-6-chloropyridine-2-carboxylate

To a solution of Example 1.68.3 (151 mg) in N,N-dimethylformamide (3 mL) was added di-tert-butyl iminodicarboxylate (54 mg). The mixture was stirred at room temperature overnight. The reaction mixture was diluted with ethyl acetate (200 mL), washed with water and brine, and dried over anhydrous sodium sulfate. Filtration and evaporation of the solvent gave the title compound, which was used in the next step without further purification. MS (ESI) m/e 729.4 (M+H)⁺.

### 1.68.5 7-(6-(tert-butoxycarbonyl)-5-(1-((3-(2-((tert-butoxycarbonyl)amino)ethoxy)-5,7-dimethyladamantan-1-yl)methyl)-5-methyl-1H-pyrazol-4-yl)pyridin-2-yl)-1-naphthoic Acid

To a solution of methyl 7-(4,4,5,5-tetramethyl-1,3,2-dioxaborolan-2-yl)-1-naphthoate (257 mg) in 1,4-dioxane (10 mL) and water (5 mL) was added Example 1.68.4 (600 mg), bis(triphenylphosphine)palladium(II) dichloride (57.8 mg), and CsF (375 mg). The mixture was stirred at 120° C. for 30 minutes under microwave conditions (Biotage Initiator). The mixture was diluted with ethyl acetate (200 mL), washed with water and brine, and dried over anhydrous sodium sulfate. Filtration and evaporation of the solvent gave crude product, which was purified by silica gel chromatography, eluting with 20% ethyl acetate in heptane to give a di-ester intermediate. The residue was dissolved in tetrahydrofuran (10 mL), methanol (5 mL) and water (5 mL) and LiOH H₂O (500 mg) was added, and the mixture was stirred at room temperature overnight. The mixture was acidified with 2N aqueous HCl, dissolved in 400 mL of ethyl acetate, washed with water and brine, and dried over anhydrous sodium sulfate. Filtration and evaporation of the solvent gave the title compound. MS (APCI) m/e 765.3 (M+H)⁺.

### 1.68.6 3-(1-((3-(2-aminoethoxy)-5,7-dimethyladamantan-1-yl)methyl)-5-methyl-1H-pyrazol-4-yl)-6-(8-(benzo[d]thiazol-2-ylcarbamoyl)naphthalen-2-yl)picolinic Acid

To a solution of Example 1.68.5 (500 mg) in dichloromethane (10 mL) was added benzo[d]thiazol-2-amine (98 mg), 1-ethyl-3-(3-dimethylaminopropyl)carbodiimide (251 mg) and 4-dimethylaminopyridine (160 mg). The mixture was stirred at room temperature overnight. The reaction mixture was diluted with ethyl acetate (400 mL), washed with water and brine, and dried over anhydrous sodium sulfate. Filtration and evaporation of the solvent gave a residue that was dissolved in dichloromethane and trifluoroacetic acid (10 mL, 1:1). After stirring overnight, the solution was concentrated under reduced pressure. The residue was dissolved in N,N-dimethylformamide (12 mL) and purified by reverse-phase HPLC (using a Gilson system and a C18 column, eluting with 20-80% acetonitrile in water containing 0.1% trifluoroacetic acid) to give the title compound. MS (ESI) m/e 741.2 (M+H)⁺.

### 1.68.7 6-[8-(1,3-benzothiazol-2-ylcarbamoyl)naphthalen-2-yl]-3-{1-[(3-{2-[(2-carboxyethyl)amino]ethoxy}-5,7-dimethyltricyclo[3.3.1.1^{3,7}]dec-1-yl)methyl]-5-methyl-1H-pyrazol-4-yl}pyridine-2-carboxylic Acid

To a solution of Example 1.68.6 (35 mg) in N,N-dimethylformamide (4 mL) was added tert-butyl acrylate (120 mg) and H₂O (138 mg). The mixture was stirred at room temperature overnight. The reaction mixture was diluted with ethyl acetate (400 mL), washed with water and brine, and dried over anhydrous sodium sulfate. Filtration and evaporation of the solvent gave a residue that was dissolved in dichloromethane and trifluoroacetic acid (10 mL, 1:1). After 16 hours, the mixture was concentrated under reduced pressure. The residue was dissolved in N,N-dimethylformamide (2 mL) and purified by reverse-phase HPLC on a Gilson system (C18 column), eluting with 20-80% acetonitrile in water containing 0.1% trifluoroacetic acid, to give the title compound. ¹H NMR (400 MHz, dimethyl sulfoxide-d₆) δ ppm 13.08 (s, 1H), 8.99 (d, 1H), 8.43-8.24 (m, 4H), 8.24-8.11 (m, 3H), 8.04 (d, 1H), 7.99 (d, 1H), 7.90 (d, 1H), 7.78 (d, 1H), 7.74-7.62 (m, 1H), 7.53-7.43 (m, 2H), 7.35 (q, 1H), 3.87 (s, 2H), 3.08 (dp, 4H), 2.62 (t, 2H), 2.20 (s, 3H), 1.43 (s, 2H), 1.29 (q, 4H), 1.14 (s, 4H), 1.03 (q, 2H), 0.85 (s, 6H).

### 1.69 Synthesis of 6-[5-(1,3-benzothiazol-2-ylcarbamoyl)quinolin-3-yl]-3-{1-[(3,5-dimethyl-7-{2-[(2-sulfoethyl)amino]ethoxy}tricyclo[3.3.1.1^{3,7}]dec-1-yl)methyl]-5-methyl-1H-pyrazol-4-yl}pyridine-2-carboxylic Acid

### 1.69.1 methyl 3-bromoquinoline-5-carboxylate

To a solution of 3-bromoquinoline-5-carboxylic acid (2 g) in methanol (30 mL) was added concentrated H₂SO₄ (5 mL). The solution was stirred at reflux overnight. The mixture was concentrated under reduced pressure. The residue was dissolved in ethyl acetate (300 mL) and washed with aqueous Na₂CO₃ solution, water and brine. After drying over anhydrous sodium sulfate, filtration and evaporation of the solvent gave the title compound. MS (ESI) m/e 266 (M+H)⁺.

### 1.69.2 methyl 3-(4,4,5,5-tetramethyl-1,3,2-dioxaborolan-2-yl)quinoline-5-carboxylate

To a solution of Example 1.69.1 (356 mg) in N,N-dimethylformamide (5 mL) was added PdCl₂(dppf)-CH₂Cl₂ adduct ([1,1′-bis(diphenylphosphino)ferrocene]dichloropalladium(II) (1:1), 55 mg) potassium acetate (197 mg) and bis(pinacolato)diboron (510 mg). The mixture was stirred at 60° C. overnight. The mixture was cooled to room temperature and used in the next reaction without further work up. MS (ESI) m/e 339.2 (M+Na)⁺.

### 1.69.3 methyl 3-[5-{1-[(3-{2-[bis(tert-butoxycarbonyl)amino]ethoxy}-5,7-dimethyltricyclo[3.3.1.1^{3,7}]dec-1-yl)methyl]-5-methyl-1H-pyrazol-4-yl}-6-(tert-butoxycarbonyl)pyridin-2-yl]quinoline-5-carboxylate

To a solution of Example 1.69.2 (626 mg) in 1,4-dioxane (10 mL) and water (5 mL) was added Example 1.68.4 (1.46 g), bis(triphenylphosphine)palladium(II) dichloride (140 mg), and CsF (911 mg). The mixture was stirred at 120° C. for 30 minutes under microwave conditions (Biotage Initiator). The mixture was diluted with ethyl acetate (200 mL), washed with water and brine, dried over anhydrous sodium sulfate, filtered and concentrated. The residue was purified by silica gel chromatography, eluting with 20% ethyl acetate in heptane (1 L) to give the title compound. MS (ESI) m/e 880.3 (M+H)⁺.

### 1.69.4 3-(6-(tert-butoxycarbonyl)-5-(1-((3-(2-((tert-butoxycarbonyl)amino)ethoxy)-5,7-dimethyladamantan-1-yl)methyl)-5-methyl-1H-pyrazol-4-yl)pyridin-2-yl)quinoline-5-carboxylic Acid

To a solution of Example 1.69.3 (1.34 g) in tetrahydrofuran (10 mL), methanol (5 mL) and water (5 mL) was added LiOH H₂O (120 mg), and the mixture was stirred at room temperature overnight. The mixture was acidified with 2N aqueous HCl, diluted with ethyl acetate (400 mL), washed with water and brine, and dried over anhydrous sodium sulfate. Filtration and evaporation of the solvent gave the title compound. MS (APCI) m/e 766.3 (M+H)⁺.

### 1.69.5 3-(1-((3-(2-aminoethoxy)-5,7-dimethyladamantan-1-yl)methyl)-5-methyl-1H-pyrazol-4-yl)-6-(5-(benzo[d]thiazol-2-ylcarbamoyl)quinolin-3-yl)picolinic Acid

To a solution of Example 1.69.4 (200 mg) in dichloromethane (10 mL) was added benzo[d]thiazol-2-amine (39.2 mg), 1-ethyl-3-(3-dimethylaminopropyl)carbodiimide (50 mg) and 4-dimethylaminopyridine (32 mg). The mixture was stirred at room temperature overnight. The reaction mixture was diluted with ethyl acetate (200 mL), washed with water and brine, dried over anhydrous sodium sulfate, filtered, and concentrated. The residue was dissolved in dichloromethane and trifluoroacetic acid (10 mL, 1:1), and the reaction was stirred overnight. The mixture was concentrated, and the residue was dissolved in N,N-dimethylformamide (12 mL) and purified by reverse-phase HPLC on a Gilson system (C18 column), eluting with 20-80% acetonitrile in water containing 0.1% trifluoroacetic acid, to give the title compound. MS (ESI) m/e 742.1 (M+H)⁺.

### 1.69.6 6-[5-(1,3-benzothiazol-2-ylcarbamoyl)quinolin-3-yl]-3-{1-[(3,5-dimethyl-7-{2-[(2-sulfoethyl)amino]ethoxy}tricyclo[3.3.1.1^{3,7}]dec-1-yl)methyl]-5-methyl-1H-pyrazol-4-yl}pyridine-2-carboxylic Acid

To a solution of Example 1.69.5 (36 mg) in N,N-dimethylformamide (2 mL) was added 4-((tert-butyldiphenylsilyl)oxy)-2,2-dimethylbutyl ethenesulfonate (22 mg) and H₂O (0.3 mL)). The mixture was stirred at room temperature for 3 hours. The reaction mixture was diluted with dichloromethane and trifluoroacetic acid (10 mL, 1:1) and stirred overnight. The mixture was concentrated, and the residue was dissolved in N,N-dimethylformamide (4 mL) and purified by reverse-phase HPLC on a Gilson system (C18 column), eluting with 20-80% acetonitrile in water containing 0.1% trifluoroacetic acid, to give the title compound. ¹H NMR (400 MHz, dimethyl sulfoxide-d₆) δ ppm 13.19 (s, 2H), 9.70 (d, 1H), 9.40 (s, 1H), 8.31 (d, 2H), 8.16 (d, 1H), 8.06 (d, 1H), 8.01 (d, 1H), 7.98-7.88 (m, 1H), 7.80 (d, 1H), 7.52-7.43 (m, 2H), 7.37 (q, 1H), 3.89 (s, 2H), 3.22 (p, 2H), 3.10 (q, 2H), 2.80 (t, 2H), 2.23 (s, 3H), 1.43 (s, 2H), 1.30 (q, 4H), 1.23-1.10 (m, 4H), 1.04 (q, 2H), 0.87 (s, 6H). MS (ESI) m/e 850.2 (M+H)⁺.

### 1.70 Synthesis of 6-[4-(1,3-benzothiazol-2-ylcarbamoyl)quinolin-6-yl]-3-{1-[(3,5-dimethyl-7-{2-[(2-sulfoethyl)amino]ethoxy}tricyclo[3.3.1.1^{3,7}]dec-1-yl)methyl]-5-methyl-1H-pyrazol-4-yl}pyridine-2-carboxylic Acid

### 1.70.1 ethyl 6-(4,4,5,5-tetramethyl-1,3,2-dioxaborolan-2-yl)quinoline-4-carboxylate

To a solution of ethyl 6-bromoquinoline-4-carboxylate (140 mg) in N,N-dimethylformamide (2 mL) was added PdCl₂(dppf)-CH₂Cl₂ adduct (([1,1′-bis(diphenylphosphino)ferrocene]dichloropalladium(II) (1:1), 20.42 mg), potassium acetate (147 mg) and bis(pinacolato)diboron (190 mg). The mixture was stirred at 60° C. overnight. The mixture was cooled to room temperature and used in the next reaction without further work up. MS (ESI) m/e 328.1 (M+H)⁺.

### 1.70.2 ethyl 6-[5-{1-[(3-{2-[bis(tert-butoxycarbonyl)amino]ethoxy}-5,7-dimethyltricyclo[3.3.1.1^{3,7}]dec-1-yl)methyl]-5-methyl-1H-pyrazol-4-yl}-6-(tert-butoxycarbonyl)pyridin-2-yl]quinoline-4-carboxylate

To a solution of Example 1.70.1 (164 mg) in 1,4-dioxane (10 mL) and water (5 mL) was added Example 1.68.4 (365 mg), bis(triphenylphosphine)palladium(II) dichloride (35 mg), and CsF (228 mg). The mixture was stirred at 120° C. for 30 minutes under microwave conditions (Biotage Initiator). The mixture was diluted with ethyl acetate (200 mL), washed with water and brine, dried over anhydrous sodium sulfate, filtered and concentrated. The residue was purified by silica gel chromatography, eluting with 20% ethyl acetate in heptane (1 L) to give the title compound. MS (ESI) m/e 894.3 (M+H)⁺.

### 1.70.3 6-(6-(tert-butoxycarbonyl)-5-(1-((3-(2-((tert-butoxycarbonyl)amino)ethoxy)-5,7-dimethyladamantan-1-yl)methyl)-5-methyl-1H-pyrazol-4-yl)pyridin-2-yl)quinoline-4-carboxylic Acid

To a solution of Example 1.70.2 (3.1 g) in tetrahydrofuran (20 mL), methanol (10 mL) and water (10 mL) was added LiOH H₂O (240 mg). The mixture was stirred at room temperature overnight. The mixture was acidified with 2N aqueous HCl and diluted with ethyl acetate (400 mL). The organic layer was washed with water and brine and dried over anhydrous sodium sulfate. Filtration and evaporation of the solvent gave the title compound. MS (ESI) m/e 766.3 (M+H)⁺.

### 1.70.4 3-(1-((3-(2-aminoethoxy)-5,7-dimethyladamantan-1-yl)methyl)-5-methyl-1H-pyrazol-4-yl)-6-(4-(benzo[d]thiazol-2-ylcarbamoyl)quinolin-6-yl)picolinic Acid

To a solution of Example 1.70.3 (4.2 g) in dichloromethane (30 mL) was added benzo[d]thiazol-2-amine (728 mg), 1-ethyl-3-(3-dimethylaminopropyl)carbodiimide (1.40 g) and 4-dimethylaminopyridine (890 mg), and the mixture was stirred at room temperature overnight. The reaction mixture was diluted with ethyl acetate (500 mL), washed with water and brine, and dried over anhydrous sodium sulfate. Filtration and evaporation of the solvent gave a residue that was dissolved in dichloromethane and trifluoroacetic acid (10 mL, 1:1) and stirred overnight. The mixture was concentrated, and the residue was dissolved in N,N-dimethylformamide (4 mL) and purified by reverse-phase HPLC on a Gilson system (C18 column), eluting with 20-80% acetonitrile in water containing 0.1% trifluoroacetic acid, to give the title compound. MS (ESI) m/e 742.2 (M+H)⁺.

### 1.70.5 6-[4-(1,3-benzothiazol-2-ylcarbamoyl)quinolin-6-yl]-3-{1-[(3,5-dimethyl-7-{2-[(2-sulfoethyl)amino]ethoxy}tricyclo[3.3.1.1^{3,7}]dec-1-yl)methyl]-5-methyl-1H-pyrazol-4-yl}pyridine-2-carboxylic Acid

To a solution of Example 1.70.4 (111 mg) in N,N-dimethylformamide (4 mL) was added 4-((tert-butyldiphenylsilyl)oxy)-2,2-dimethylbutylethenesulfonate (67 mg), N,N-diisopropylethylamine (0.2 mL) and H₂O (0.3 mL). The mixture was stirred at room temperature for 3 hours. The reaction mixture was diluted with dichloromethane and trifluoroacetic acid (10 mL, 1:1) and stirred overnight. The mixture was concentrated, and the residue was dissolved in N,N-dimethylformamide (4 mL) and purified by reverse-phase HPLC on a Gilson system (C18 column), eluting with 20-80% acetonitrile in water containing 0.1% trifluoroacetic acid, to give the title compound. ¹H NMR (400 MHz, dimethyl sulfoxide-d₆) δ ppm 13.31 (s, 1H), 9.10 (d, 1H), 8.91 (s, 1H), 8.58 (dd, 1H), 8.47-8.16 (m, 4H), 8.06 (dd, 1H), 7.99-7.89 (m, 2H), 7.79 (d, 1H), 7.53-7.43 (m, 2H), 7.42-7.31 (m, 1H), 3.87 (s, 2H), 3.53 (d, 1H), 3.20 (p, 2H), 3.07 (p, 2H), 2.78 (t, 2H), 2.20 (s, 3H), 1.40 (s, 2H), 1.28 (q, 4H), 1.21-1.07 (m, 4H), 1.02 (q, 2H), 0.84 (s, 6H). MS (ESI) m/e 850.1 (M+H)⁺.

### 1.71 Synthesis of 6-[5-(1,3-benzothiazol-2-ylcarbamoyl)quinolin-3-yl]-3-{1-[(3-{2-[(2-carboxyethyl)amino]ethoxy}-5,7-dimethyltricyclo[3.3.1.1^{3,7}]dec-1-yl)methyl]-5-methyl-1H-pyrazol-4-yl}pyridine-2-carboxylic Acid

To a solution of Example 1.69.5 (140 mg) in N,N-dimethylformamide (10 mL) was added tert-butyl acrylate (242 mg), and H₂O (0.3 mL), and the mixture was stirred at room temperature over the weekend. The reaction mixture was diluted with dichloromethane and trifluoroacetic acid (10 mL, 1:1) and stirred overnight. The mixture was concentrated, and the residue was dissolved in N,N-dimethylformamide (4 mL) and purified by reverse-phase HPLC on a Gilson system (C18 column), eluting with 20-80% acetonitrile in water containing 0.1% trifluoroacetic acid, to give the title compound. ¹H NMR (400 MHz, dimethyl sulfoxide-d₆) δ ppm 13.17 (s, 2H), 9.69 (d, 1H), 9.37 (d, 1H), 8.30 (dd, 3H), 8.15 (dd, 1H), 8.04 (dd, 1H), 7.99-7.88 (m, 2H), 7.79 (d, 1H), 7.53-7.40 (m, 2H), 7.34 (td, 1H), 3.88 (s, 2H), 3.55 (t, 2H), 3.08 (dt, 4H), 2.62 (t, 2H), 2.21 (s, 3H), 1.43 (s, 2H), 1.29 (q, 4H), 1.14 (s, 4H), 1.03 (q, 2H), 0.85 (s, 6H). MS (ESI) m/e 814.2 (M+H)⁺.

### 1.72 Synthesis of 6-[1-(1,3-benzothiazol-2-ylcarbamoyl)-5,6-dihydroimidazo[1,5-a]pyrazin-7(8H)-yl]-3-{1-[(3,5-dimethyl-7-{2-[(2-sulfoethyl)amino]ethoxy}tricyclo[3.3.1.1^{3,7}]dec-1-yl)methyl]-5-methyl-1H-pyrazol-4-yl}pyridine-2-carboxylic Acid

### 1.72.1 ethyl 7-(5-bromo-6-(tert-butoxycarbonyl)pyridin-2-yl)-5,6,7,8-tetrahydroimidazo[1,5-a]pyrazine-1-carboxylate

The title compound was prepared by substituting ethyl 5,6,7,8-tetrahydroimidazo[1,5-a]pyrazine-1-carboxylate hydrochloride for 1,2,3,4-tetrahydroisoquinoline-8-carboxylate hydrochloride in Example 1.1.11. MS (ESI) m/e 451, 453 (M+H)⁺, 395, 397 (M-tert-butyl)⁺.

### 1.72.2 ethyl 7-(6-(tert-butoxycarbonyl)-5-(4,4,5,5-tetramethyl-1,3,2-dioxaborolan-2-yl)pyridin-2-yl)-5,6,7,8-tetrahydroimidazo[1,5-a]pyrazine-1-carboxylate

The title compound was prepared by substituting Example 1.72.1 for Example 1.1.11 in Example 1.2.1. MS (ESI) m/e 499 (M+H)⁺, 443 (M-tert-butyl)⁺, 529 (M+CH₃OH−H)⁻.

### 1.72.3 ethyl 7-(6-(tert-butoxycarbonyl)-5-(1-((3-(2-((tert-butoxycarbonyl)amino)ethoxy)-5,7-dimethyladamantan-1-yl)methyl)-5-methyl-1H-pyrazol-4-yl)pyridin-2-yl)-5,6,7,8-tetrahydroimidazo[1,5-a]pyrazine-1-carboxylate

The title compound was prepared by substituting Example 1.72.2 for Example 1.2.1 and Example 1.55.11 for Example 1.13.3 in Example 1.13.4. MS (ESI) m/e 760 (M+H)⁺, 758 (M−H)⁻.

### 1.72.4 7-(6-(tert-butoxycarbonyl)-5-(1-((3-(2-((tert-butoxycarbonyl)amino)ethoxy)-5,7-dimethyladamantan-1-yl)methyl)-5-methyl-1H-pyrazol-4-yl)pyridin-2-yl)-5,6,7,8-tetrahydroimidazo[1,5-a]pyrazine-1-carboxylic Acid

The title compound was prepared by substituting Example 1.72.3 for Example 1.1.12 in Example 1.1.13. MS (ESI) m/e 760 (M+H)⁺, 758 (M−H)⁻.

### 1.72.5 tert-butyl 6-(1-(benzo[d]thiazol-2-ylcarbamoyl)-5,6-dihydroimidazo[1,5-a]pyrazin-7(8H)-yl)-3-(1-((3-(2-((tert-butoxycarbonyl)amino)ethoxy)-5,7-dimethyladamantan-1-yl)methyl)-5-methyl-1H-pyrazol-4-yl)picolinate

The title compound was prepared by substituting Example 1.72.4 for Example 1.52.2 in Example 1.52.3. MS (ESI) m/e 892 (M+H)⁺, 890 (M−H)⁻.

### 1.72.6 3-(1-{[3-(2-aminoethoxy)-5,7-dimethyltricyclo[3.3.1.1^{3,7}]dec-1-yl]methyl}-5-methyl-1H-pyrazol-4-yl)-6-[1-(1,3-benzothiazol-2-ylcarbamoyl)-5,6-dihydroimidazo[1,5-a]pyrazin-7(8H)-yl]pyridine-2-carboxylic Acid

The title compound was prepared by substituting Example 1.72.5 for Example 1.1.16 in Example 1.1.17. MS (ESI) m/e 736 (M+H)⁺, 734 (M−H)⁻.

### 1.72.7 6-(1-(benzo[d]thiazol-2-ylcarbamoyl)-5,6-dihydroimidazo[1,5-a]pyrazin-7(8H)-yl)-3-(1-((3-(2-((2-(((4-((tert-butyldiphenylsilyl)oxy)-2-methylbutan-2-yl)oxy)sulfonyl)ethyl)amino)ethoxy)-5,7-dimethyladamantan-1-yl)methyl)-5-methyl-1H-pyrazol-4-yl)picolinic Acid

The title compound was prepared by substituting Example 1.72.6 for Example 1.2.7 in Example 1.2.8.

### 1.72.8 6-[1-(1,3-benzothiazol-2-ylcarbamoyl)-5,6-dihydroimidazo[1,5-a]pyrazin-7(8H)-yl]-3-{1-[(3,5-dimethyl-7-{2-[(2-sulfoethyl)amino]ethoxy}tricyclo[3.3.1.1^{3,7}]dec-1-yl)methyl]-5-methyl-1H-pyrazol-4-yl}pyridine-2-carboxylic Acid

The title compound was prepared by substituting Example 1.72.7 for Example 1.2.8 in Example 1.2.9. ¹H NMR (400 MHz, dimethyl sulfoxide-d₆) δ ppm 8.36 (bs, 2H), 8.03 (bs, 1H), 7.99 (d, 1H), 7.76 (d, 1H), 7.64 (d, 1H), 7.46 (t, 1H), 7.34 (s, 1H), 7.33 (t, 1H), 7.17 (d, 1H), 5.12 (s, 2H), 4.28 (t, 2H), 4.11 (t, 2H), 3.86 (s, 2H), 3.56 (t, 2H), 3.24 (m, 2H), 3.11 (m, 2H), 2.82 (t, 2H), 2.15 (s, 3H), 1.42 (s, 2H), 1.32 (q, 4H), 1.17 (q, 4, H), 1.03 (m, 2H), 0.88 (s, 6H). MS (ESI) m/e 844 (M+H)⁺, 842 (M−H)⁻.

### 1.73 Synthesis of 8-(1,3-benzothiazol-2-ylcarbamoyl)-2-{6-carboxy-5-[1-({3-[2-({3-[1-(beta-D-glucopyranuronosyl)-1H-1,2,3-triazol-4-yl]propyl}amino)ethoxy]-5,7-dimethyltricyclo[3.3.1.1^{3,7}]dec-1-yl}methyl)-5-methyl-1H-pyrazol-4-yl]pyridin-2-yl}-1,2,3,4-tetrahydroisoquinoline

To a solution of (2R,3R,4S,5S,6S)-2-azido-6-(methoxycarbonyl)tetrahydro-2H-pyran-3,4,5-triyl triacetate (8.63 mg) in t-CH₃OH (2 mL) and water (1 mL) was added Example 1.67.1 (20 mg), copper(II) sulfate pentahydrate (2.0 mg) and sodium ascorbate (5 mg). The mixture was stirred for 20 minutes at 100° C. under microwave conditions (Biotage Initiator). LiOH H₂O (50 mg) was added to the mixture, and stirring was continued overnight. The mixture was neutralized with trifluoroacetic acid and purified by reverse-phase HPLC on a Gilson system (C18 column), eluting with 20-80% acetonitrile in water containing 0.1% trifluoroacetic acid, to give the title compound. MS (APCI) m/e 987.3 (M+H)⁺.

### 1.74 Synthesis of 6-[7-(1,3-benzothiazol-2-ylcarbamoyl)-1H-indol-2-yl]-3-{1-[(3,5-dimethyl-7-{2-[(2-sulfoethyl)amino]ethoxy}tricyclo[3.3.1.1^{3,7}]dec-1-yl)methyl]-5-methyl-1H-pyrazol-4-yl}pyridine-2-carboxylic Acid

### 1.74.1 methyl 2-[5-{1-[(3-{2-[bis(tert-butoxycarbonyl)amino]ethoxy}-5,7-dimethyltricyclo[3.3.1.1^{3,7}]dec-1-yl)methyl]-5-methyl-1H-pyrazol-4-yl}-6-(tert-butoxycarbonyl)pyridin-2-yl]-1H-indole-7-carboxylate

Example 1.74.1 was prepared by substituting methyl 2-(4,4,5,5-tetramethyl-1,3,2-dioxaborolan-2-yl)-1H-indole-7-carboxylate for Example 1.2.1 and substituting Example 1.68.4 for Example 1.1.6 in Example 1.1.12. MS (ESI) m/e 866.3 (M−H)⁻.

### 1.74.2 2-(6-(tert-butoxycarbonyl)-5-(1-((3-(2-((tert-butoxycarbonyl)amino)ethoxy)-5,7-dimethyladamantan-1-yl)methyl)-5-methyl-1H-pyrazol-4-yl)pyridin-2-yl)-1H-indole-7-carboxylic Acid

Example 1.74.2 was prepared by substituting Example 1.74.1 for Example 1.1.12 in Example 1.1.13. MS (ESI) m/e 754.4 (M+H)⁺.

### 1.74.3 tert-butyl 6-(7-(benzo[d]thiazol-2-ylcarbamoyl)-1H-indol-2-yl)-3-(1-((3-(2-((tert-butoxycarbonyl)amino)ethoxy)-5,7-dimethyladamantan-1-yl)methyl)-5-methyl-1H-pyrazol-4-yl)picolinate

Example 1.74.3 was prepared by substituting Example 1.74.2 for Example 1.1.13 in Example 1.1.14. MS (ESI) m/e 886.5 (M+H)⁺.

### 1.74.4 3-(1-((3-(2-aminoethoxy)-5,7-dimethyladamantan-1-yl)methyl)-5-methyl-1H-pyrazol-4-yl)-6-(7-(benzo[d]thiazol-2-ylcarbamoyl)-1H-indol-2-yl)picolinic acid

Example 1.74.4 was prepared by substituting Example 1.74.3 for Example 1.1.16 in Example 1.1.17. MS (ESI) m/e 730.2 (M+H)⁺.

### 1.74.5 6-[7-(1,3-benzothiazol-2-ylcarbamoyl)-1H-indol-2-yl]-3-[1-({3,5-dimethyl-7-[(2,2,7,7-tetramethyl-10,10-dioxido-3,3-diphenyl-4,9-dioxa-10l⁶-thia-13-aza-3-silapentadecan-15-yl)oxy]tricyclo[3.3.1.1^{3,7}]dec-1-yl}methyl)-5-methyl-1H-pyrazol-4-yl]pyridine-2-carboxylic Acid

Example 1.74.5 was prepared by substituting Example 1.74.4 for Example 1.2.7 in Example 1.2.8. MS (ESI) m/e 1176.7 (M+H)⁺.

### 1.74.6 6-[7-(1,3-benzothiazol-2-ylcarbamoyl)-1H-indol-2-yl]-3-{1-[(3,5-dimethyl-7-{2-[(2-sulfoethyl)amino]ethoxy}tricyclo[3.3.1.1^{3,7}]dec-1-yl)methyl]-5-methyl-1H-pyrazol-4-yl}pyridine-2-carboxylic Acid

Example 1.74.6 was prepared by substituting Example 1.74.5 for Example 1.2.8 in Example 1.2.9. ¹H NMR (400 MHz, dimethyl sulfoxide-d₆) δ ppm 11.32 (d, 1H), 8.23 (dd, 1H), 8.18 (d, 1H), 7.93-7.82 (m, 3H), 7.71 (d, 1H), 7.62 (s, 3H), 7.57-7.51 (m, 1H), 7.47 (s, 1H), 7.40 (d, 1H), 7.35 (t, 1H), 7.22 (t, 1H), 4.86 (t, 2H), 3.85 (s, 2H), 3.47 (t, 2H), 3.08 (t, 2H), 2.88 (p, 2H), 2.21 (s, 3H), 1.37 (s, 2H), 1.32-1.20 (m, 4H), 1.14 (q, 4H), 1.07-0.94 (m, 2H), 0.84 (s, 6H). MS (ESI) m/e 838.2 (M+H)⁺.

### 1.75 Synthesis of 6-[8-(1,3-benzothiazol-2-ylcarbamoyl)-6-[3-(methylamino)propyl]-3,4-dihydroisoquinolin-2(1H)-yl]-3-{1-[(3,5-dimethyl-7-{2-[(2-sulfoethyl)amino]ethoxy}tricyclo[3.3.1.1^{3,7}]dec-1-yl)methyl]-5-methyl-1H-pyrazol-4-yl}pyridine-2-carboxylic Acid

### 1.75.1 methyl 3-bromo-5-(bromomethyl)benzoate

Azobisisobutyronitrile (1.79 g) was added to methyl 3-bromo-5-methylbenzoate (50 g) and N-bromosuccinimide (44.7 g) in 350 mL acetonitrile, and the mixture was refluxed overnight. An additional 11 g of N-bromosuccinimide and 0.5 g of azobisisobutyronitrile was added, and the refluxing was continued for 3 hours. The mixture was concentrated, taken up in 500 mL diethyl ether, and stirred for 30 minutes. The mixture was filtered, and the resulting solution was concentrated. The crude product was chromatographed on silica gel using 10% ethyl acetate in heptanes to give the title compound.

### 1.75.2 methyl 3-bromo-5-(cyanomethyl)benzoate

Tetrabutylammonium cyanide (50 g) was added to Example 1.75.1 (67.1 g) in 300 mL acetonitrile, and the mixture was heated to 70° C. overnight. The mixture was cooled, poured into diethyl ether, and rinsed with water and brine. The mixture was then concentrated and chromatographed on silica gel using 2-20% ethyl acetate in heptanes to give the title compound.

### 1.75.3 methyl 3-(2-aminoethyl)-5-bromobenzoate

Borane-THF complex (126 mL, 1M solution) was added to a solution of Example 1.75.2 (16 g) in 200 mL tetrahydrofuran, and the mixture was stirred overnight. The reaction was carefully quenched with methanol (50 mL), and then concentrated to 50 mL volume. The mixture was taken up in 120 mL methanol/120 mL 4M HCl/120 mL dioxane, and stirred overnight. The organics were removed under reduced pressure, and the residue was extracted twice with diethyl ether. The extracts were discarded. The organic layer was basified with solid K₂CO₃, and then extracted with ethyl acetate, and dichloromethane (2×). The extracts were combined, dried over Na₂SO₄, filtered and concentrated to give the title compound.

### 1.75.4 methyl 3-bromo-5-(2-(2,2,2-trifluoroacetamido)ethyl)benzoate

Trifluoroacetic anhydride (9.52 mL) was added dropwise to a mixture of Example 1.75.3 (14.5 g) and trimethylamine (11.74 mL) in 200 mL dichloromethane at 0° C. Upon addition the mixture was allowed to warm to room temperature and was stirred for three days. The mixture was poured into diethyl ether, and washed with NaHCO₃ solution and brine. The mixture was concentrated and chromatographed on silica gel using 5-30% ethyl acetate in heptanes to give the title compound.

### 1.75.5 methyl 6-bromo-2-(2,2,2-trifluoroacetyl)-1,2,3,4-tetrahydroisoquinoline-8-carboxylate

Sulfuric acid was added to Example 1.75.4 (10 g) until it went into solution (40 mL), at which time paraformaldehyde (4.24 g) was added and the mixture was stirred for 2 hours. The solution was then poured onto 400 mL ice, and stirred 10 minutes. The mixture was extracted with ethyl acetate (3×), and the combined extracts were washed with NaHCO₃ solution and brine, and then concentrated The crude product was chromatographed on silica gel using 2-15% ethyl acetate in heptanes to give the title compound.

### 1.75.6 methyl 6-(3-((tert-butoxycarbonyl)(methyl)amino)prop-1-yn-1-yl)-2-(2,2,2-trifluoroacetyl)-1,2,3,4-tetrahydroisoquinoline-8-carboxylate

A solution of Example 1.75.5 (5.1 g), tert-butyl methyl(prop-2-yn-1-yl)carbamate (2.71 g), bis(triphenylphosphine)palladium(II) dichloride (PdCl₂(PPh₃)₂, 0.49 g), CuI (0.106 g), and triethylamine (5.82 mL) was stirred in 50 mL dioxane at 50° C. overnight. The mixture was concentrated and chromatographed on silica gel using 10-50% ethyl acetate in heptanes to give the title compound.

### 1.75.7 methyl 6-(3-((tert-butoxycarbonyl)(methyl)amino)propyl)-2-(2,2,2-trifluoroacetyl)-1,2,3,4-tetrahydroisoquinoline-8-carboxylate

Example 1.75.6 (4.2 g), tetrahydrofuran (20 mL) and methanol (20.00 mL) were added to wet 20% Pd(OH)₂/C (3 g) in a 250 mL pressure bottle and shaken under a pressure of 50 psi and 50° C. for 12 hours. The solution was filtered and concentrated to give the title compound.

### 1.75.8 methyl 2-(5-bromo-6-(tert-butoxycarbonyl)pyridin-2-yl)-6-(3-((tert-butoxycarbonyl)(methyl)amino)propyl)-1,2,3,4-tetrahydroisoquinoline-8-carboxylate

Example 1.75.7 (4.22 g), and potassium carbonate (1.53 g) were stirred in 60 mL tetrahydrofuran, 25 mL methanol, and 10 mL water overnight. The mixture was concentrated and 60 mL N,N-dimethylformamide was added. To this was then added Example 1.1.9 (3.05 g) and triethylamine (5 mL), and the reaction was stirred at 60° C. overnight. The mixture was cooled to room temperature, poured into ethyl acetate (600 mL), washed with water (3×) and brine, dried over Na₂SO₄, filtered, and concentrated. The residue was chromatographed on silica gel using 5-50% ethyl acetate in heptanes to give the title compound. MS (ESI) m/e 618.2 (M+H)⁺.

### 1.75.9 methyl 6-(3-((tert-butoxycarbonyl)(methyl)amino)propyl)-2-(6-(tert-butoxycarbonyl)-5-(4,4,5,5-tetramethyl-1,3,2-dioxaborolan-2-yl)pyridin-2-yl)-1,2,3,4-tetrahydroisoquinoline-8-carboxylate

To a solution of Example 1.75.8 (3.7 g), triethylamine (2.50 mL) and PdCl₂(dppf) (([1,1′-bis(diphenylphosphino)ferrocene]dichloropalladium(II) (1:1), 0.29 g) in 25 mL acetonitrile was added 4,4,5,5-tetramethyl-1,3,2-dioxaborolane (1.74 mL), and the reaction mixture was heated to 75° C. for 5 hours, then stirred at 60° C. overnight. The mixture was concentrated and chromatographed on silica gel using 5-50% ethyl acetate in heptanes to give the title compound. MS (ESI) m/e 666.4 (M+H)⁺.

### 1.75.10 4-((tert-butyldiphenylsilyl)oxy)-2,2-dimethylbutyl 2-((2-((3-((4-iodo-5-methyl-1H-pyrazol-1-yl)methyl)-5,7-dimethyladamantan-1-yl)oxy)ethyl)amino)ethanesulfonate

Example 1.55.10 (2.39 g), 4-((tert-butyldiphenylsilyl)oxy)-2,2-dimethylbutyl ethenesulfonate (2.41 g), and triethylamine (1.51 mL) were stirred in 30 mL N,N-dimethylformamide at 45° C. for 3 hours. The mixture was cooled and poured into diethyl ether (400 mL), and the diethyl ether solution was washed with water (3×) and brine, and concentrated. The crude product was chromatographed on silica gel using 2-50% ethyl acetate in heptanes, with 1% added triethylamine to give the title compound. MS (ESI) m/e 890.6 (M+H)⁺.

### 1.75.11 6-(6-(3-((tert-butoxycarbonyl)(methyl)amino)propyl)-8-(methoxycarbonyl)-3,4-dihydroisoquinolin-2(1H)-yl)-3-(1-((3-(2-((2-((4-((tert-butyldiphenylsilyl)oxy)-2,2-dimethylbutoxy)sulfonyl)ethyl)amino)ethoxy)-5,7-dimethyladamantan-1-yl)methyl)-5-methyl-1H-pyrazol-4-yl)picolinic Acid

Example 1.75.9 (1.777 g), Example 1.75.10 (1.98 g), tris(dibenzylideneacetone)dipalladium(0) (0.102 g), 1,3,5,7-tetramethyl-8-tetradecyl-2,4,6-trioxa-8-phosphaadamantane (0.918 g), and potassium phosphate (1.889 g) were added to 25 mL dioxane/10 mL water, and the solution was evacuated/filled with nitrogen several times. The reaction was clear, and was stirred at 70° C. overnight. The mixture was cooled and poured into ethyl acetate (200 mL), and washed with water and brine. The mixture was concentrated and chromatographed on silica gel using 5-50% ethyl acetate in heptanes, followed by 10% methanol in ethyl acetate with 1% triethylamine to give the title compound. MS (ESI) m/e 1301.4 (M+H)⁺.

### 1.75.12 6-(3-((tert-butoxycarbonyl)(methyl)amino)propyl)-2-(5-(1-((3-(2-((2-((4-((tert-butyldiphenylsilyl)oxy)-2,2-dimethylbutoxy)sulfonyl)ethyl)amino)ethoxy)-5,7-dimethyladamantan-1-yl)methyl)-5-methyl-1H-pyrazol-4-yl)-6-carboxypyridin-2-yl)-1,2,3,4-tetrahydroisoquinoline-8-carboxylic Acid

Example 1.75.11 (1.5 g) and LiOH—H₂O (0.096 g) were stirred in 15 mL tetrahydrofuran and 3 mL water at 45° C. for 10 days. The mixture was poured into 200 mL ethyl acetate/20 mL NaH₂PO₄ solution, and concentrated HCl solution was added until the pH reached 3. The layers were separated, and the aqueous layer was extracted twice with ethyl acetate. The combined organic layers were washed with brine and concentrated. The residue was chromatographed on silica gel using 0-5% methanol in ethyl acetate to give the title compound. MS (ESI) m/e 1287.3 (M+H)⁺.

### 1.75.13 6-(8-(benzo[d]thiazol-2-ylcarbamoyl)-6-(3-((tert-butoxycarbonyl)(methyl)amino)propyl)-3,4-dihydroisoquinolin-2(1H)-yl)-3-(1-((3-(2-((2-((4-((tert-butyldiphenylsilyl)oxy)-2,2-dimethylbutoxy)sulfonyl)ethyl)amino)ethoxy)-5,7-dimethyladamantan-1-yl)methyl)-5-methyl-1H-pyrazol-4-yl)picolinic Acid

The title compound was prepared as described in Example 1.2.6, substituting Example 1.2.5 with Example 1.75.12. MS (ESI) m/e 1419.5 (M+H)⁺.

### 1.75.14 6-[8-(1,3-benzothiazol-2-ylcarbamoyl)-6-[3-(methylamino)propyl]-3,4-dihydroisoquinolin-2(1H)-yl]-3-{1-[(3,5-dimethyl-7-{2-[(2-sulfoethyl)amino]ethoxy}tricyclo[3.3.1.1^{3,7}]dec-1-yl)methyl]-5-methyl-1H-pyrazol-4-yl}pyridine-2-carboxylic Acid

The title compound was prepared as described in Example 1.2.9, substituting Example 1.2.8 with Example 1.75.13. ¹H NMR (400 MHz, dimethyl sulfoxide-d₆) δ ppm 12.90 (bs, 1H), 8.33 (m, 2H), 8.02 (d, 1H), 7.78 (d, 1H), 7.66 (m, 1H), 7.47 (m, 3H), 7.35 (m, 3H), 7.25 (s, 2H), 6.95 (d, 1H), 4.95 (s, 2H), 4.28 (t, 2H), 4.11 (t, 2H), 3.95 (m, 2H), 3.20 (m, 2H), 3.08 (m, 2H), 2.96 (m, 2H), 2.89 (m, 2H), 2.78 (m, 2H), 2.65 (m, 2H), 2.55 (t, 2H), 2.12 (s, 3H), 1.95 (m, 2H), 1.39 (s, 2H), 1.25 (m, 6H), 1.12 (m, 6H), 0.93 (s, 3H), 0.85 (s, 6H). MS (ESI) m/e 926.8 (M+H)⁺.

### 1.76 Synthesis of 5-{[2-({3-[(4-{6-[8-(1,3-benzothiazol-2-ylcarbamoyl)-3,4-dihydroisoquinolin-2(1H)-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl]amino}-5-deoxy-D-arabinitol

### 1.76.1 tert-butyl 6-(8-(benzo[d]thiazol-2-ylcarbamoyl)-3,4-dihydroisoquinolin-2(1H)-yl)-3-(1-((3,5-dimethyl-7-(2-((((4R,4′R,5R)-2,2,2′,2′-tetramethyl-[4,4′-bi(1,3-dioxolan)]-5-yl)methyl)amino)ethoxy)adamantan-1-yl)methyl)-5-methyl-1H-pyrazol-4-yl)picolinate

Example 1.2.7 (75 mg) and (4R,4′R,5S)-2,2,2′,2′-tetramethyl-[4,4′-bi(1,3-dioxolane)]-5-carbaldehyde (22 mg) were dissolved in dichloromethane (1 mL). Sodium triacetoxyborohydride (40 mg) was added, and the solution was stirred for 16 hours at room temperature. The solution was concentrated under reduced pressure, and the material was purified by flash column chromatography on silica gel, eluting with 5-10% methanol in dichloromethane. The solvent was evaporated under reduced pressure to provide the title compound. MS (ESI) m/e 1016 (M+H)⁺, 1014 (M−H)⁻.

### 1.76.2 5-{[2-({3-[(4-{6-[8-(1,3-benzothiazol-2-ylcarbamoyl)-3,4-dihydroisoquinolin-2(1H)-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl]amino}-5-deoxy-D-arabinitol

Example 1.76.1 (45 mg) was dissolved in trifluoroacetic acid (1 mL) and water (0.2 mL). The solution was mixed at room temperature for five days. The solvents were removed under reduced pressure, and the material was taken up in methanol (2 mL). The material was purified by reverse-phase HPLC using 25-75% acetonitrile in water (w/ 0.1% TFA) over 30 minutes on a Grace Reveleris equipped with a Luna column: C18(2), 100 A, 250×30 mm. Product fractions were pooled, frozen, and lyophilized to yield the title compound as the bis trifluoroacetic acid salt. ¹H NMR (400 MHz, dimethyl sulfoxide-d₆) δ ppm 12.85 (bs, 2H), 8.31 (m, 1H), 8.16 (m, 1H), 8.04 (d, 1H), 7.80 (d, 1H), 7.62 (d, 1H), 7.51-7.43 (m, 3H), 7.37 (q, 2H), 7.29 (s, 1H), 6.69 (d, 1H), 4.96 (s, 2H), 4.04 (t, 2H), 3.89 (m, 2H), 3.59 (m, 3H), 3.49 (m, 4H), 3.42 (dd, 2H), 3.22 (dd, 2H), 3.06 (m, 2H), 3.02 (m, 4H), 2.10 (s, 3H), 1.43 (s, 2H), 1.30 (q, 4H), 1.14 (t, 4H), 1.04 (q, 2H), 0.87 (s, 6H). MS (ESI) m/e 880 (M+H)⁺, 878 (M−H)⁻.

### 1.77 Synthesis of 1-{[2-({3-[(4-{6-[8-(1,3-benzothiazol-2-ylcarbamoyl)-3,4-dihydroisoquinolin-2(1H)-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl]amino}-1,2-dideoxy-D-arabino-hexitol

### 1.77.1 tert-butyl 6-(8-(benzo[d]thiazol-2-ylcarbamoyl)-3,4-dihydroisoquinolin-2(1H)-yl)-3-(1-((3,5-dimethyl-7-(2-(((3R,4S,5R)-3,4,5,6-tetrahydroxyhexyl)amino)ethoxy)adamantan-1-yl)methyl)-5-methyl-1H-pyrazol-4-yl)picolinate

(4R,5S,6R)-6-(Hydroxymethyl)tetrahydro-2H-pyran-2,4,5-triol (15 mg) was dissolved in dimethyl sulfoxide (0.5 mL). Example 1.2.7 (88 mg) was added, followed by sodium cyanoborohydride (27 mg). Acetic acid (82 mg) was added dropwise, and the solution was heated at 60° C. for 16 hours. The reaction was cooled, diluted with 1 mL of methanol, and purified by reverse-phase HPLC using 20-75% acetonitrile in water (w/ 0.1% TFA) over 60 minutes on a Grace Reveleris equipped with a Luna column: C18(2), 100 A, 150×30 mm. Product fractions were pooled, frozen, and lyophilized to yield the title compound as the bis trifluoroacetic acid salt. MS (ESI) m/e 950 (M+H)⁺, 948 (M−H)⁻.

### 1.77.2 1-{[2-({3-[(4-{6-[8-(1,3-benzothiazol-2-ylcarbamoyl)-3,4-dihydroisoquinolin-2(1H)-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl]amino}-1,2-dideoxy-D-arabino-hexitol

Example 1.77.1 (39 mg) was dissolved in dichloromethane (0.5 mL). Trifluoroacetic acid (740 mg) was added, and the solution was stirred at room temperature for 16 hours. The solvents were removed under reduced pressure. The residue was dissolved in N,N-dimethylformamide (0.5 mL) and 1 M aqueous sodium hydroxide (0.5 mL) was added. The solution was stirred at room temperature for one hour. Trifluoroacetic acid (0.25 mL) was added, and the material was purified by reverse-phase HPLC using 20-75% acetonitrile in water (w/ 0.1% TFA) over 60 minutes on a Grace Reveleris equipped with a Luna column: C18(2), 100 A, 150×30 mm. Product fractions were pooled, frozen, and lyophilized to yield the title compound as the bis trifluoroacetic acid salt. ¹H NMR (400 MHz, dimethyl sulfoxide-d₆) δ ppm 12.86 (s, 1H), 12.74 (bs, 1H), 8.28 (bs, 1H), 8.20 (bs, 1H), 8.04 (d, 1H), 7.80 (d, 1H), 7.62 (d, 1H), 7.51-7.43 (m, 3H), 7.37 (q, 2H), 7.29 (s, 1H), 6.96 (d, 1H), 4.96 (s, 2H), 4.53 (bs, 3H), 3.89 (t, 2H), 3.83 (s, 2H), 3.77 (d, 1H), 3.60 (dd, 2H), 3.56 (t, 2H), 3.48 (m, 2H), 3.15 (d, 1H), 3.02 (m, 6H), 2.10 (s, 3H), 1.84 (m, 1H), 1.69 (m, 1H), 1.43 (s, 2H), 1.31 (q, 4H), 1.14 (t, 4H), 1.05 (q, 2H), 0.87 (s, 6H). MS (ESI) m/e 894 (M+H)⁺, 892 (M−H)⁻.

### 1.78 Synthesis of 6-[4-(1,3-benzothiazol-2-ylcarbamoyl)isoquinolin-6-yl]-3-{1-[(3,5-dimethyl-7-{2-[(2-sulfoethyl)amino]ethoxy}tricyclo[3.3.1.1^{3,7}]dec-1-yl)methyl]-5-methyl-1H-pyrazol-4-yl}pyridine-2-carboxylic Acid

### 1.78.1 methyl 6-(4,4,5,5-tetramethyl-1,3,2-dioxaborolan-2-yl)isoquinoline-4-carboxylate

To a solution of methyl 6-bromoisoquinoline-4-carboxylate (1.33 g) in N,N-dimethylformamide (30 mL) was added PdCl₂(dppf)-CH₂Cl₂ adduct (([1,1′-bis(diphenylphosphino)ferrocene]dichloropalladium(II) (1:1), 204 mg), potassium acetate (1.48 g) and bis(pinacolato)diboron (1.92 g). The mixture was stirred at 60° C. overnight. The mixture was cooled to room temperature and used in the next reaction without further work up. MS (APCI) m/e 313.3 (M+H)⁺.

### 1.78.2 methyl 6-[5-{1-[(3-{2-[bis(tert-butoxycarbonyl)amino]ethoxy}-5,7-dimethyltricyclo[3.3.1.1^{3,7}]dec-1-yl)methyl]-5-methyl-1H-pyrazol-4-yl}-6-(tert-butoxycarbonyl)pyridin-2-yl]isoquinoline-4-carboxylate

To a solution of the Example 1.68.4 (1.2 g) in 1,4-dioxane (20 mL) and water (10 mL) was added Example 1.78.1 (517 mg), bis(triphenylphosphine)palladium(II) dichloride (58 mg), and CsF (752 mg). The mixture was stirred at reflux overnight. LC/MS showed the expected product as a major peak. The mixture was diluted with ethyl acetate (200 mL), washed with water and brine, dried over anhydrous sodium sulfate, filtered and concentrated. The residue was purified by silica gel chromatography, eluting with 20% ethyl acetate in dichloromethane to give the title compound. MS (ESI) m/e 880.8 (M+H)⁺.

### 1.78.3 6-(6-(tert-butoxycarbonyl)-5-(1-((3-(2-((tert-butoxycarbonyl)amino)ethoxy)-5,7-dimethyladamantan-1-yl)methyl)-5-methyl-1H-pyrazol-4-yl)pyridin-2-yl)isoquinoline-4-carboxylic Acid

To a solution of Example 1.78.2 (3.1 g) in tetrahydrofuran (20 mL), methanol (10 mL) and water (10 mL) was added LiOH H₂O (240 mg). The mixture was stirred at room temperature overnight. The mixture was acidified with aqueous 2N HCl and diluted with ethyl acetate (400 mL). The organic layer was washed with water and brine and dried over anhydrous sodium sulfate. Filtration and evaporation of the solvent gave the title compound. MS (ESI) m/e 766.4 (M+H)⁺.

### 1.78.4 3-(1-((3-(2-aminoethoxy)-5,7-dimethyladamantan-1-yl)methyl)-5-methyl-1H-pyrazol-4-yl)-6-(4-(benzo[d]thiazol-2-ylcarbamoyl)isoquinolin-6-yl)picolinic Acid

To a solution of Example 1.78.3 (1.2 g) in dichloromethane (20 mL) was added benzo[d]thiazol-2-amine (0.236 g), 1-ethyl-3-(3-dimethylaminopropyl)carbodiimide (451 mg), and 4-dimethylaminopyridine (288 mg), and the mixture was stirred at room temperature overnight. The reaction mixture was diluted with ethyl acetate (500 mL), washed with water and brine, and dried over anhydrous sodium sulfate. Filtration and evaporation of the solvent gave a residue that was dissolved in dichloromethane and trifluoroacetic acid (10 mL, 1:1) and stirred overnight. The mixture was concentrated, and the residue was dissolved in N,N-dimethylformamide (4 mL) and purified by reverse-phase HPLC on a Gilson system (C18 column), eluting with 20-80% acetonitrile in water containing 0.1% trifluoroacetic acid, to give the title compound. MS (ESI) m/e 742.1 (M+H)⁺.

### 1.78.5 6-[4-(1,3-benzothiazol-2-ylcarbamoyl)isoquinolin-6-yl]-3-{1-[(3,5-dimethyl-7-{2-[(2-sulfoethyl)amino]ethoxy}tricyclo[3.3.1.1^{3,7}]dec-1-yl)methyl]-5-methyl-1H-pyrazol-4-yl}pyridine-2-carboxylic Acid

To a solution of Example 1.78.4 (55 mg) in N,N-dimethylformamide (6 mL) was added 4-((tert-butyldiphenylsilyl)oxy)-2,2-dimethylbutyl ethenesulfonate (34 mg), N,N-diisopropylethylamine (0.6 mL) and H₂O (0.6 mL). The mixture was stirred at room temperature overnight. The reaction mixture was diluted with dichloromethane and trifluoroacetic acid (10 mL, 1:1) and stirred overnight. The mixture was concentrated, and the residue was dissolved in N,N-dimethylformamide (4 mL) and purified by reverse-phase HPLC on a Gilson system (C18 column), eluting with 20-80% acetonitrile in water containing 0.1% trifluoroacetic acid, to give the title compound. ¹H NMR (400 MHz, dimethyl sulfoxide-d₆) δ ppm 13.25 (s, 2H), 9.58 (s, 1H), 9.06 (s, 1H), 9.00 (s, 1H), 8.52 (dd, 1H), 8.42 (d, 1H), 8.35 (d, 2H), 8.26 (d, 1H), 8.11-8.03 (m, 1H), 8.01 (d, 1H), 7.80 (d, 1H), 7.52-7.44 (m, 2H), 7.41-7.28 (m, 1H), 3.89 (s, 2H), 3.55 (t, 2H), 3.22 (t, 2H), 3.09 (s, 2H), 2.80 (t, 2H), 2.23 (s, 3H), 1.43 (s, 2H), 1.30 (q, 4H), 1.23-1.11 (m, 4H), 1.04 (q, 2H), 0.86 (s, 6H). MS (ESI+) m/e 850.1 (M+H)⁺.

### 1.79 Synthesis of 6-[8-(1,3-benzothiazol-2-ylcarbamoyl)-3,4-dihydroisoquinolin-2(1H)-yl]-3-(1-{[3-(2-{[3-hydroxy-2-(hydroxymethyl)propyl]amino}ethoxy)-5,7-dimethyltricyclo[3.3.1.1^{3,7}]dec-1-yl]methyl}-5-methyl-1H-pyrazol-4-yl)pyridine-2-carboxylic Acid

### 1.79.1 2,2-dimethyl-1,3-dioxane-5-carbaldehyde

To a stirred suspension of pyridinium chlorochromate (1.1 g) and diatomaceous earth (10 g) in dichloromethane (10 mL) was added (2,2-dimethyl-1,3-dioxan-5-yl)methanol (0.5 g) as a solution in dichloromethane (3 mL) dropwise. The mixture was stirred at room temperature for 2 hours. The suspension was filtered through diatomaceous earth and washed with ethyl acetate. The crude product was filtered through silica gel and concentrated to give the title compound. ¹H NMR (501 MHz, chloroform-d) δ 9.89 (s, 1H), 4.28-4.17 (m, 4H), 2.42-2.32 (m, 1H), 1.49 (s, 3H), 1.39 (s, 3H). MS (ESI) m/e 305.9 (2M+NH4)⁺.

### 1.79.2 tert-butyl 6-(8-(benzo[d]thiazol-2-ylcarbamoyl)-3,4-dihydroisoquinolin-2(1H)-yl)-3-(1-((3-(2-(((2,2-dimethyl-1,3-dioxan-5-yl)methyl)amino)ethoxy)-5,7-dimethyladamantan-1-yl)methyl)-5-methyl-1H-pyrazol-4-yl)picolinate

To a solution of Example 1.2.7 (100 mg) and Example 1.79.1 (20 mg) in dichloromethane (1 mL) was added sodium triacetoxyborohydride (40 mg), and the mixture was stirred at room temperature for 2 hours. The reaction was diluted with dichloromethane and washed with saturated sodium bicarbonate solution. The aqueous layer was back extracted with dichloromethane. The combined organic layers were dried over sodium sulfate, filtered and concentrated. Purification of the residue by silica gel chromatography, eluting with 20%-100% ethyl acetate/ethanol (3:1) in heptane, provided the title compound. MS (ESI) m/e 930.3 (M+H)⁺.

### 1.79.3 6-[8-(1,3-benzothiazol-2-ylcarbamoyl)-3,4-dihydroisoquinolin-2(1H)-yl]-3-(1-{[3-(2-{[3-hydroxy-2-(hydroxymethyl)propyl]amino}ethoxy)-5,7-dimethyltricyclo[3.3.1.1^{3,7}]dec-1-yl]methyl}-5-methyl-1H-pyrazol-4-yl)pyridine-2-carboxylic Acid

Example 1.79.3 was prepared by substituting Example 1.79.2 for Example 1.2.8 in Example 1.2.9. ¹H NMR (400 MHz, dimethyl sulfoxide-d₆) δ ppm 12.82 (s, 1H), 8.13 (s, 2H), 8.00 (dd, 1H), 7.76 (d, 1H), 7.59 (d, 1H), 7.49-7.38 (m, 3H), 7.37-7.29 (m, 2H), 7.25 (s, 1H), 6.92 (d, 1H), 4.92 (s, 4H), 3.85 (t, 2H), 3.79 (s, 2H), 3.53 (t, 2H), 3.47 (dd, 2H), 3.00 (dt, 7H), 2.07 (s, 3H), 1.93 (p, 1H), 1.38 (s, 2H), 1.32-1.19 (m, 4H), 1.16-0.91 (m, 6H), 0.83 (s, 7H). MS (ESI) m/e 834.3 (M+H)⁺.

### 1.80 Synthesis of 1-{[2-({3-[(4-{6-[8-(1,3-benzothiazol-2-ylcarbamoyl)-3,4-dihydroisoquinolin-2(1H)-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl]amino}-1,2-dideoxy-D-erythro-pentitol

The title compound was prepared by substituting (4S,5R)-tetrahydro-2H-pyran-2,4,5-triol for (4R,5S,6R)-6-(hydroxymethyl)tetrahydro-2H-pyran-2,4,5-triol and Example 1.3.1 for Example 1.2.7 in Example 1.77.1. ¹H NMR (400 MHz, dimethyl sulfoxide-d₆) δ ppm 12.85 (bs, 1H), 12.72 (bs, 1H), 8.21 (bs, 2H), 8.04 (d, 1H), 7.79 (d, 1H), 7.62 (d, 1H), 7.52-7.42 (m, 3H), 7.37 (q, 2H), 7.29 (s, 1H), 6.95 (d, 1H), 4.96 (s, 2H), 3.89 (t, 2H), 3.83 (s, 2H), 3.65 (m, 2H), 3.56 (m, 2H), 3.38 (m, 2H), 3.32 (m, 2H), 3.24 (m, 2H), 3.03 (m, 5H), 2.10 (s, 3H), 1.89 (m, 1H), 1.67 (m, 1H), 1.44 (s, 2H), 1.31 (q, 4H), 1.14 (t, 4H), 1.05 (q, 2H), 0.86 (s, 6H). MS (ESI) m/e 864 (M+H)⁺, 862 (M−H)⁻.

### 1.81 Synthesis of 6-[8-(1,3-benzothiazol-2-ylcarbamoyl)-3,4-dihydroisoquinolin-2(1H)-yl]-3-(1-{[3,5-dimethyl-7-(2-{[(2S,3S)-2,3,4-trihydroxybutyl]amino}ethoxy)tricyclo[3.3.1.1^{3,7}]dec-1-yl]methyl}-5-methyl-1H-pyrazol-4-yl)pyridine-2-carboxylic Acid

### 1.81.1 Carbonic Acid Tert-Butyl ester (4S,5S)-5-hydroxymethyl-2,2-dimethyl-[1,3]dioxolan-4-ylmethyl Ester

((4S,5S)-2,2-Dimethyl-1,3-dioxolane-4,5-diyl)dimethanol (1000 mg) was dissolved in N,N-dimethylformamide (50 mL). Sodium hydride (60% in mineral oil, 259 mg) was added. The solution was mixed at room temperature for 15 minutes. Di-tert-butyl dicarbonate (1413 mg) was added slowly. The solution was mixed for 30 minutes, and the reaction was quenched with saturated aqueous ammonium chloride solution. The solution was diluted with water (150 mL) and extracted twice using 70% ethyl acetate in heptanes. The organic portions were combined and extracted with water (100 mL), extracted with brine (50 mL), and dried on anhydrous sodium sulfate. The solution was concentrated under reduced pressure, and the material was purified by flash column chromatography on silica gel, eluting with 30% ethyl acetate in heptanes. The solvent was evaporated under reduced pressure to provide the title compound. MS (ESI) m/e 284 (M+Na)⁺.

### 1.81.2 Carbonic Acid Tert-Butyl ester (4S,5R)-5-formyl-2,2-dimethyl-[1,3]dioxolan-4-ylmethyl Ester

Example 1.81.1 (528 mg) was dissolved in dichloromethane (20 mL). Dess-Martin periodinane (896 mg) was added, and the solution was stirred at room temperature for four hours. The solution was concentrated under reduced pressure, and the material was purified by flash column chromatography on silica gel, eluting with 20%-50% ethyl acetate in heptanes. The solvent was evaporated under reduced pressure to provide the title compound.

### 1.81.3 tert-butyl 6-(8-(benzo[d]thiazol-2-ylcarbamoyl)-3,4-dihydroisoquinolin-2(1H)-yl)-3-(1-(((1 S,3s,5R,7S)-3-(2-((((4S,5S)-5-(((tert-butoxycarbonyl)oxy)methyl)-2,2-dimethyl-1,3-dioxolan-4-yl)methyl)amino)ethoxy)-5,7-dimethyladamantan-1-yl)methyl)-5-methyl-1H-pyrazol-4-yl)picolinate

The title compound was prepared by substituting Example 1.81.2 for (4R,4′R,5S)-2,2,2′,2′-tetramethyl-[4,4′-bi(1,3-dioxolane)]-5-carbaldehyde in Example 1.76.1.

### 1.81.4 6-[8-(1,3-benzothiazol-2-ylcarbamoyl)-3,4-dihydroisoquinolin-2(1H)-yl]-3-(1-{[3,5-dimethyl-7-(2-{[(2S,3S)-2,3,4-trihydroxybutyl]amino}ethoxy)tricyclo[3.3.1.1^{3,7}]dec-1-yl]methyl}-5-methyl-1H-pyrazol-4-yl)pyridine-2-carboxylic Acid

The title compound was prepared by substituting Example 1.81.3 for Example 1.76.1 in Example 1.76.2. ¹H NMR (400 MHz, dimethyl sulfoxide-d₆) δ ppm 12.86 (bs, 2H), 8.28 (bs, 1H), 8.18 (bs, 1H), 8.04 (d, 1H), 7.80 (d, 1H), 7.63 (d, 1H), 7.51-7.43 (m, 3H), 7.36 (q, 2H), 7.29 (s, 1H), 6.96 (d, 1H), 4.96 (s, 2H), 3.89 (t, 2H), 3.83 (m, 3H), 3.46 (m, 4H), 3.40 (m, 4H), 3.08-2.96 (m, 6H), 2.10 (s, 3H), 1.43 (s, 2H), 1.30 (q, 4H), 1.14 (t, 4H), 1.04 (q, 2H), 0.87 (s, 6H). MS (ESI) m/e 850 (M+H)⁺, 848 (M−H)⁻.

### 1.82 Synthesis of 6-[8-(1,3-benzothiazol-2-ylcarbamoyl)-3,4-dihydroisoquinolin-2(1H)-yl]-3-(1-{[3-(2-{[(2S,3S,4R,5R,6R)-2,3,4,5,6,7-hexahydroxyheptyl]amino}ethoxy)-5,7-dimethyltricyclo[3.3.1.1^{3,7}]dec-1-yl]methyl}-5-methyl-1H-pyrazol-4-yl)pyridine-2-carboxylic Acid

The title compound was prepared by substituting (2R,3R,4S,5R,6R)-2,3,4,5,6,7-hexahydroxyheptanal for (4R,5S,6R)-6-(hydroxymethyl)tetrahydro-2H-pyran-2,4,5-triol and Example 1.3.1 for Example 1.2.7 in Example 1.77.1. ¹H NMR (400 MHz, dimethyl sulfoxide-d₆) δ ppm 12.86 (bs, 1H), 8.34-8.08 (m, 2H), 8.05 (d, 1H), 7.79 (d, 1H), 7.54-7.43 (m, 3H), 7.37 (m, 2H), 7.30 (s, 1H), 6.95 (d, 1H), 4.96 (s, 2H), 3.93 (m, 2H), 3.90 (m, 4H), 3.83 (s, 2H), 3.47 (m, 4H), 3.41 (m, 4H), 3.18-3.08 (m, 7H), 3.03 (t, 2H), 2.12 (s, 3H), 1.46 (s, 2H), 1.28 (q, 4H), 1.15 (t, 4H), 1.05 (q, 2H), 0.89 (s, 6H). MS (ESI) m/e 940 (M+H)⁺.

### 1.83 Synthesis of 6-[8-(1,3-benzothiazol-2-ylcarbamoyl)-3,4-dihydroisoquinolin-2(1H)-yl]-3-{1-[(3-{2-[({3-[(1,3-dihydroxypropan-2-yl)amino]propyl}sulfonyl)amino]ethoxy}-5,7-dimethyltricyclo[3.3.1.1^{3,7}]dec-1-yl)methyl]-5-methyl-1H-pyrazol-4-yl}pyridine-2-carboxylic Acid

### 1.83.1 tert-butyl 6-(8-(benzo[d]thiazol-2-ylcarbamoyl)-3,4-dihydroisoquinolin-2(1H)-yl)-3-(1-((3-(2-(3-((1,3-dihydroxypropan-2-yl)amino)propylsulfonamido)ethoxy)-5,7-dimethyladamantan-1-yl)methyl)-5-methyl-1H-pyrazol-4-yl)picolinate

To a cooled (ice bath) solution of Example 1.2.7 (31 mg) and N,N-diisopropylethylamine (60 μL) in dichloromethane (1 mL) was added 3-chloropropane-1-sulfonyl chloride (5 μL). The mixture was stirred at room temperature for 2 hours. The reaction was concentrated, dissolved in N,N-dimethylformamide (1 mL), transferred to a 2 mL microwave tube and 2-aminopropane-1,3-diol (70 mg) was added. The mixture was heated at 130° C. under microwave conditions (Biotage Initiator) for 90 minutes. The reaction mixture was concentrated, and the residue was purified by reverse-phase HPLC using a Gilson system, eluting with 20-100% acetonitrile in water containing 0.1% v/v trifluoroacetic acid. The desired fractions were combined and freeze-dried to provide the title compound. MS (ESI) m/e 997.2 (M+H)⁺.

### 1.83.2 6-[8-(1,3-benzothiazol-2-ylcarbamoyl)-3,4-dihydroisoquinolin-2(1H)-yl]-3-{1-[(3-{2-[({3-[(1,3-dihydroxypropan-2-yl)amino]propyl}sulfonyl)amino]ethoxy}-5,7-dimethyltricyclo[3.3.1.1^{3,7}]dec-1-yl)methyl]-5-methyl-1H-pyrazol-4-yl}pyridine-2-carboxylic Acid

Example 1.83.2 was prepared by substituting Example 1.83.1 for Example 1.2.8 in Example 1.2.9. ¹H NMR (400 MHz, dimethyl sulfoxide-d₆) δ ppm 12.84 (s, 1H), 8.40 (s, 2H), 8.05-7.98 (m, 1H), 7.77 (d, 1H), 7.60 (d, 1H), 7.51-7.39 (m, 3H), 7.38-7.30 (m, 2H), 7.27 (s, 1H), 7.13 (t, 1H), 6.93 (d, 1H), 4.94 (s, 2H), 3.61 (qd, 4H), 3.36 (t, 2H), 3.16-2.93 (m, 10H), 2.08 (s, 3H), 2.00 (p, 2H), 1.38 (s, 2H), 1.25 (q, 4H), 1.15-0.92 (m, 6H), 0.84 (s, 6H). MS (ESI) m/e 941.2 (M+H)⁺.

### 1.84 Synthesis of 6-[8-(1,3-benzothiazol-2-ylcarbamoyl)-3,4-dihydroisoquinolin-2(1H)-yl]-3-{1-[(3-{2-[(3-{[1,3-dihydroxy-2-(hydroxymethyl)propan-2-yl]amino}-3-oxopropyl)amino]ethoxy}-5,7-dimethyltricyclo[3.3.1.1^{3,7}]dec-1-yl)methyl]-5-methyl-1H-pyrazol-4-yl}pyridine-2-carboxylic Acid

To a solution of tert-butyl 3-(1-((3-(2-aminoethoxy)-5,7-dimethyladamantan-1-yl)methyl)-5-methyl-1H-pyrazol-4-yl)-6-(8-(benzo[d]thiazol-2-ylcarbamoyl)-3,4-dihydroisoquinolin-2(1H)-yl)picolinate (55 mg) in N,N-dimethylformamide (6 mL) was added N-(1,3-dihydroxy-2-(hydroxymethyl)propan-2-yl)acrylamide (73.4 mg), N,N-diisopropylethylamine (0.2 mL) and H₂O (0.2 mL). The mixture was stirred at room temperature 4 days. LC/MS showed the expected product as a major peak. The reaction mixture was diluted with ethyl acetate (500 mL), washed with water and brine, and dried over anhydrous sodium sulfate. Filtration and evaporation of the solvent gave a residue that was dissolved in dichloromethane and trifluoroacetic acid (10 mL, 1:1) and stirred overnight. The mixture was concentrated, and the residue was dissolved in N,N-dimethylformamide (8 mL) and purified by reverse-phase HPLC on a Gilson system (C18 column), eluting with 20-80% acetonitrile in water containing 0.1% trifluoroacetic acid, to give the title compound. ¹H NMR (400 MHz, dimethylsulfoxide-d₆) δ ppm 12.84 (s, 1H), 8.45 (s, 2H), 8.01 (d, 4H), 7.78 (d, 1H), 7.60 (d, 1H), 7.53-7.39 (m, 3H), 7.39-7.30 (m, 2H), 7.27 (s, 1H), 6.94 (d, 1H), 4.94 (s, 2H), 4.14 (s, 2H), 3.87 (t, 2H), 3.81 (s, 2H), 3.52 (d, 4H), 3.19 (s, 3H), 3.13-2.97 (m, 5H), 2.75 (t, 2H), 2.08 (s, 3H), 1.42 (s, 2H), 1.29 (q, 4H), 1.12 (s, 4H), 1.09-0.99 (m, 2H), 0.85 (s, 7H). MS (ESI) m/e 921.2 (M+H)⁺.

### 1.85 Synthesis of 6-[8-(1,3-benzothiazol-2-ylcarbamoyl)-3,4-dihydroisoquinolin-2(1H)-yl]-3-(1-{[3-(2-{[(3S)-3,4-dihydroxybutyl]amino}ethoxy)-5,7-dimethyltricyclo[3.3.1.1^{3,7}]dec-1-yl]methyl}-5-methyl-1H-pyrazol-4-yl)pyridine-2-carboxylic Acid

To a solution of Example 1.2.7 (213 mg) in dichloromethane (2 mL) was added (S)-2-(2,2-dimethyl-1,3-dioxolan-4-yl)acetaldehyde (42 mg). After stirring at room temperature for 30 minutes, sodium triacetoxyborohydride (144 mg) was added. The reaction mixture was stirred at room temperature overnight. Trifluoroacetic acid (2 mL) was added and stirring was continued overnight. The reaction mixture was concentrated, and the residue was purified by reverse-phase HPLC using a Gilson system, eluting with 5-85% acetonitrile in water containing 0.1% v/v trifluoroacetic acid. The desired fractions were combined and freeze-dried to provide the title compound. ¹H NMR (400 MHz, dimethyl sulfoxide-d₆) δ ppm 12.86 (s, 1H), 8.22 (d, 2H), 8.05-8.01 (m, 1H), 7.79 (d, 1H), 7.61 (d, 1H), 7.53-7.41 (m, 3H), 7.36 (td, 2H), 7.28 (s, 1H), 6.95 (d, 1H), 4.95 (s, 2H), 3.88 (t, 2H), 3.82 (s, 2H), 3.26-2.94 (m, 7H), 2.10 (s, 3H), 1.84-1.75 (m, 1H), 1.52-1.63 (m, 1H), 1.45-1.23 (m, 6H), 1.19-0.96 (m, 7H), 0.86 (s, 6H). MS (ESI) m/e 834.3 (M+H)⁺.

### 1.86 Synthesis of 4-({[2-({3-[(4-{6-[8-(1,3-benzothiazol-2-ylcarbamoyl)-3,4-dihydroisoquinolin-2(1H)-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl]amino}methyl)phenyl Beta-D-Glucopyranosiduronic Acid

To a solution of 3-(1-((3-(2-aminoethoxy)-5,7-dimethyladamantan-1-yl)methyl)-5-methyl-1H-pyrazol-4-yl)-6-(8-(benzo[d]thiazol-2-ylcarbamoyl)-3,4-dihydroisoquinolin-2(1H)-yl)picolinic acid (36 mg) in tetrahydrofuran (2 mL) and acetic acid (0.2 mL) was added (2S,3R,4S,5S,6S)-2-(4-formylphenoxy)-6-(methoxycarbonyl)tetrahydro-2H-pyran-3,4,5-triyl triacetate (21 mg) followed by MgSO₄ (60 mg). The mixture was stirred at room temperature for 1 hour before the addition of MP-cyanoborohydride (Biotage, 153 mg, 2.49 mmol/g). The mixture was then stirred at room temperature for 3 hours. The mixture was filtered, and LiOH H₂O (20 mg) was added to the filtrate. The mixture was stirred at room temperature for 2 hours and then acidified with trifluoroacetic acid. The solution was purified by reverse-phase HPLC on a Gilson system (C18 column), eluting with 20-80% acetonitrile in water containing 0.1% trifluoroacetic acid, to give the title compound. MS (ESI) m/e 1028.3 (M+H)⁺.

### 1.87 Synthesis of 3-{[2-({3-[(4-{6-[8-(1,3-benzothiazol-2-ylcarbamoyl)naphthalen-2-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl]amino}propyl Beta-D-Glucopyranosiduronic Acid

### 1.87.1 (2R,3R,5S,6S)-2-(3-hydroxypropoxy)-6-(methoxycarbonyl)tetrahydro-2H-pyran-3,4,5-triyl Triacetate

To a stirred solution of (2R,3R,5S,6S)-2-bromo-6-(methoxycarbonyl)tetrahydro-2H-pyran-3,4,5-triyl triacetate (3.98 g) in toluene (60 mL) was added propane-1,3-diol (15.22 g). The mixture was stirred at 75° C., and Ag₂CO₃ (5.52 g) was added in three portions over a period of 3 hours. The mixture was stirred at room temperature overnight, after which the suspension was filtered. The filtrate was concentrated, and the residue was purified by silica gel chromatography eluting with 50% ethyl acetate in heptane to give the title compound. MS (ESI) m/e 409.9 (M+NH₄)⁺.

### 1.87.2 (2S,3S,5R,6R)-2-(methoxycarbonyl)-6-(3-oxopropoxy)tetrahydro-2H-pyran-3,4,5-triyl Triacetate

To a solution of dimethyl sulfoxide (0.5 mL) in dichloromethane (10 mL) at −78° C. was added oxalyl chloride (0.2 mL). The mixture was stirred 20 minutes at −78° C., and a solution of Example 1.87.1 (393 mg) in dichloromethane (10 mL) was added through a syringe. After 20 minutes, triethylamine (1 mL) was added. The mixture was stirred for 30 minutes, and the temperature was allowed to rise to room temperature. The reaction mixture was diluted with ethyl acetate (300 mL), washed with water and brine, and dried over anhydrous sodium sulfate. Filtration and evaporation of the solvent gave the title compound, which was used without further purification. MS (DCI) m/e 408.1 (M+NH₄)⁺.

### 1.87.3 3-{[2-({3-[(4-{6-[8-(1,3-benzothiazol-2-ylcarbamoyl)naphthalen-2-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl]amino}propyl Beta-D-Glucopyranosiduronic Acid

To a solution of Example 1.68.6 (171 mg) in dichloromethane (10 mL) was added Example 1.87.2 (90 mg), and NaBH(OAc)₃ (147 mg). The mixture was stirred at room temperature overnight. The reaction mixture was diluted with ethyl acetate (200 mL), washed with 2% aqueous HCl solution, water, and brine, dried over anhydrous sodium sulfate, filtered and concentrated. The residue was dissolved in tetrahydrofuran (6 mL), methanol (3 mL) and water (3 mL) and LiOH H₂O (100 mg) was added. The mixture was stirred at room temperature for 2 hours, acidified with trifluoroacetic acid and concentrated under reduced pressure. The residue was dissolved in dimethyl sulfoxide/methanol (1:1, 12 mL) and purified by reverse-phase HPLC on a Gilson system (C18 column), eluting with 20-80% acetonitrile in water containing 0.1% trifluoroacetic acid) to give the title compound. ¹H NMR (400 MHz, dimethylsulfoxide-d₆) δ ppm 13.07 (s, 2H), 8.99 (s, 1H), 8.34 (dd, 1H), 8.29-8.11 (m, 5H), 8.06-8.02 (m, 1H), 7.99 (d, 1H), 7.90 (d, 1H), 7.78 (d, 1H), 7.68 (dd, 1H), 7.55-7.40 (m, 2H), 7.34 (td, 1H), 4.23 (d, 1H), 3.87 (s, 2H), 3.76 (dt, 1H), 3.60 (d, 1H), 3.53 (dt, 3H), 3.29 (t, 1H), 3.15 (t, 1H), 3.06-2.91 (m, 6H), 2.20 (s, 3H), 1.83 (p, 2H), 1.44 (s, 2H), 1.30 (q, 4H), 1.14 (s, 4H), 1.03 (q, 2H), 0.85 (s, 7H). MS (ESI) m/e 975.2 (M+H)⁺.

### 1.88 Synthesis of 6-[4-(1,3-benzothiazol-2-ylcarbamoyl)-2-oxidoisoquinolin-6-yl]-3-[1-({3,5-dimethyl-7-[2-(methylamino)ethoxy]tricyclo[3.3.1.1^{3,7}]dec-1-yl}methyl)-5-methyl-1H-pyrazol-4-yl]pyridine-2-carboxylic Acid

### 1.88.1 methyl 6-(6-(tert-butoxycarbonyl)-5-(1-((3-(2-((tert-butoxycarbonyl)(methyl)amino)ethoxy)-5,7-dimethyladamantan-1-yl)methyl)-5-methyl-1H-pyrazol-4-yl)pyridin-2-yl)isoquinoline-4-carboxylate

To a solution of Example 1.78.1 (0.73 g) in 1,4-dioxane (20 mL) and water (10 mL) was added tert-butyl 3-(1-((3-(2-((tert-butoxycarbonyl)(methyl)amino)ethoxy)-5,7-dimethyladamantan-1-yl)methyl)-5-methyl-1H-pyrazol-4-yl)-6-chloropicolinate (1.5 g), bis(triphenylphosphine)palladium(II) dichloride (82 mg), and CsF (1.06 g), and the reaction was stirred at reflux overnight. The mixture was diluted with ethyl acetate (200 mL), washed with water and brine, dried over anhydrous sodium sulfate, filtered, and concentrated. The residue was purified by silica gel chromatography, eluting with 20% ethyl acetate in heptane (1 L) to give the title compound. MS (ESI) m/e 794.8 (M+H)⁺.

### 1.88.2 6-(6-(tert-butoxycarbonyl)-5-(1-((3-(2-((tert-butoxycarbonyl)(methyl)amino)ethoxy)-5,7-dimethyladamantan-1-yl)methyl)-5-methyl-1H-pyrazol-4-yl)pyridin-2-yl)isoquinoline-4-carboxylic Acid

To a solution of Example 1.88.1 (300 mg) in tetrahydrofuran (6 mL), methanol (3 mL) and water (3 mL) was added LiOH H₂O (100 mg). The mixture was stirred at room temperature for 2 hours. The mixture was acidified with aqueous 2N HCl solution, diluted with ethyl acetate (300 mL), washed with water and brine, dried over anhydrous sodium sulfate, filtered and concentrated to give the title compound, which was used without further purification. MS (ESI) m/e 781.2 (M+H)⁺.

### 1.88.3 tert-butyl 6-(4-(benzo[d]thiazol-2-ylcarbamoyl)isoquinolin-6-yl)-3-(1-((3-(2-((tert-butoxycarbonyl)(methyl)amino)ethoxy)-5,7-dimethyladamantan-1-yl)methyl)-5-methyl-1H-pyrazol-4-yl)picolinate

To a solution of Example 1.88.2 (350 mg) in dichloromethane (10 mL) was added benzo[d]thiazol-2-amine (67.5 mg), 1-ethyl-3-(3-dimethylaminopropyl)carbodiimide (129 mg), and 4-dimethylaminopyridine (82 mg). The mixture was stirred at room temperature overnight. The mixture was diluted with ethyl acetate (300 mL), washed with water and brine, and dried over anhydrous sodium sulfate. Filtration and evaporation of the solvent gave a residue, which was purified by silica gel chromatography, eluting with 5% methanol in dichloromethane, to give the title compound. MS (APCI) m/e 912.3 (M+H)⁺.

### 1.88.4 4-(benzo[d]thiazol-2-ylcarbamoyl)-6-(6-carboxy-5-(1-((3,5-dimethyl-7-(2-(methylamino)ethoxy)adamantan-1-yl)methyl)-5-methyl-1H-pyrazol-4-yl)pyridin-2-yl)isoquinoline 2-oxide

To a solution of Example 1.88.3 (100 mg) in dichloromethane (6 mL) was added m-chloroperoxybenzoic acid (19 mg). The mixture was stirred at room temperature for 4 hours. The mixture was diluted with ethyl acetate (200 mL), washed with saturated aqueous NaHCO₃ solution, water, and brine, and dried over anhydrous sodium sulfate. Filtration and evaporation of the solvent gave a residue that was dissolved in dichloromethane/trifluoroacetic acid (10 mL, 1:1) and stirred at room temperature overnight. The solvents were evaporated, and the residue was purified by reverse-phase HPLC on a Gilson system (C18 column), eluting with 20-80% acetonitrile in water containing 0.1% trifluoroacetic acid, to give the title compound. ¹H NMR (501 MHz, dimethyl sulfoxide-d₆) δ ppm 13.32 (s, 2H), 9.21 (d, 1H), 8.71 (d, 1H), 8.49 (dd, 1H), 8.36-8.19 (m, 4H), 8.12 (dd, 1H), 8.07 (d, 1H), 7.96 (dd, 1H), 7.82 (d, 1H), 7.56-7.46 (m, 3H), 7.42-7.35 (m, 1H), 3.90 (d, 3H), 3.56 (td, 3H), 3.02 (p, 3H), 2.55 (t, 4H), 2.29-2.19 (m, 4H), 1.45 (d, 3H), 1.37-1.26 (m, 5H), 1.16 (d, 6H), 1.10-1.01 (m, 3H), 0.88 (d, 8H). MS (ESI) m/e 772.1 (M+H)⁺.

### 1.89 Synthesis of 6-{8-[(1,3-benzothiazol-2-yl)carbamoyl]-3,4-dihydroisoquinolin-2(1H)-yl}-3-{1-[(3,5-dimethyl-7-{2-[(2-sulfoethyl)amino]acetamido}tricyclo[3.3.1.1^{3,7}]decan-1-yl)methyl]-5-methyl-1H-pyrazol-4-yl}pyridine-2-carboxylic Acid

### 1.89.1 1-((3-bromo-5,7-dimethyladamantan-1-yl)methyl)-5-methyl-1H-pyrazole

To a cooled (−30° C.) solution of Example 1.1.3 (500 mg) in tetrahydrofuran (30 mL) was added n-butyllithium (9.67 mL), and the mixture was stirred at −30° C. for 2 hours. Methyl iodide (1.934 mL) was added dropwise at −30° C. After completion of the addition, the mixture was stirred at −30° C. for additional 2 hours. 1N aqueous HCl in ice water was added slowly, such that the temperature was maintained below 0° C., until the pH reached 6. The mixture was stirred at room temperature for 10 minutes, and diluted with ice-water (10 mL) and ethyl acetate (20 mL). The layers were separated, and the aqueous layer was extracted twice with ethyl acetate. The combined organic phases were washed with brine, dried over MgSO₄, filtered and concentrated. The residue was purified by flash silica gel chromatography, eluting with 15/1 to 10/1 petroleum/ethyl acetate, to give the title compound. MS (LC-MS) m/e 337, 339 (M+H)⁺.

### 1.89.2 1-(3,5-dimethyl-7-((5-methyl-1H-pyrazol-1-yl)methyl)adamantan-1-yl)urea

Example 1.89.1 (2.7 g) and urea (4.81 g) was mixed and stirred at 140° C. for 16 hours. The mixture was cooled to room temperature and suspended in methanol (200 mL×2). The insoluble material was removed by filtration. The filtrate was concentrated to give the title compound. MS (LC-MS) m/e 317.3 (M+H)⁺.

### 1.89.3 3,5-dimethyl-7-((5-methyl-1H-pyrazol-1-yl)methyl)adamantan-1-amine

To a solution of Example 1.40.2 (2.53 g) in 20% ethanol in water (20 mL) was added sodium hydroxide (12.79 g). The mixture was stirred at 120° C. for 16 hours and at 140° C. for another 16 hours. 6N Aqueous HCl was added until pH 6. The mixture was concentrated, and the residue was suspended in methanol (200 mL). The insoluble material was filtered off. The filtrate was concentrated to give the title compound as an HCl salt. MS (LC-MS) m/e 273.9 (M+H)⁺.

### 1.89.4 tert-butyl (2-((3,5-dimethyl-7-((5-methyl-1H-pyrazol-1-yl)methyl)adamantan-1-yl)amino)-2-oxoethyl)carbamate

To a solution of Example 1.89.3 (2.16 g) in N,N-dimethylformamide (100 mL) was added triethylamine (3.30 mL), 2-((tert-butoxycarbonyl)amino)acetic acid (1.799 g) and 1-[bis(dimethylamino)methylene]-1H-1,2,3-triazolo[4,5-b]pyridinium 3-oxide hexafluorophosphate (3.90 g). The mixture was stirred at room temperature for 2 hours. Water (40 mL) was added, and the mixture was extracted with ethyl acetate (70 mL×2). The combined organic phases were washed with brine, dried over sodium sulfate, filtered and concentrated. The residue was purified by silica gel chromatography, eluting with 3/1 to 2/1 petroleum/ethyl acetate, to give the title compound. MS (LC-MS) m/e 430.8 (M+H)⁺.

### 1.89.5 tert-butyl (2-((3-((4-iodo-5-methyl-1H-pyrazol-1-yl)methyl)-5,7-dimethyladamantan-1-yl)amino)-2-oxoethyl)carbamate

To an ambient solution of Example 1.89.4 (1.7 g) in N,N-dimethylformamide (20 mL) was added NIS (N-iodosuccinimide, 1.066 g) in portions, and the mixture was stirred at room temperature for 16 hours. Ice-water (10 mL) and saturated aqueous Na₂S₂O₃ solution (10 mL) were added. The mixture was extracted with ethyl acetate (30 mL×2). The combined organic phases were washed with brine, dried over sodium sulfate, filtered and concentrated. The residue was purified by silica gel chromatography, eluting with 3/1 to 2/1 petroleum/ethyl acetate, to give the title compound. MS (LC-MS) m/e 556.6 (M+H)⁺.

### 1.89.6 methyl 2-(5-bromo-6-(tert-butoxycarbonyl)pyridin-2-yl)-1,2,3,4-tetrahydroisoquinoline-8-carboxylate

To a solution of methyl 1,2,3,4-tetrahydroisoquinoline-8-carboxylate hydrochloride (12.37 g) and Example 1.1.10 (15 g) in dimethyl sulfoxide (100 mL) was added N,N-diisopropylethylamine (12 mL), and the mixture was stirred at 50° C. for 24 hours. The mixture was then diluted with ethyl acetate (500 mL) and washed with water and brine. The organic layer was dried over sodium sulfate, filtered and concentrated under reduced pressure. The residue was purified by silica gel chromatography, eluting with 20% ethyl acetate in hexane, to give the title compound. MS (ESI) m/e 448.4 (M+H)⁺.

### 1.89.7 methyl 2-(6-(tert-butoxycarbonyl)-5-(4,4,5,5-tetramethyl-1,3,2-dioxaborolan-2-yl)pyridin-2-yl)-1,2,3,4-tetrahydroisoquinoline-8-carboxylate

To a solution of Example 1.89.6 (2.25 g) and [1,1′-bis(diphenylphosphino)ferrocene]dichloropalladium(II)(205 mg) in acetonitrile (30 mL) was added triethylamine (3 mL) and pinacolborane (2 mL), and the mixture was stirred at reflux for 3 hours. The mixture was diluted with ethyl acetate (200 mL) and washed with water and brine. The organic layer was dried over sodium sulfate, filtered and concentrated under reduced pressure. Purification of the residue by flash chromatography, eluting with 20% ethyl acetate in hexane, provided the title compound.

### 1.89.8 methyl 2-(6-(tert-butoxycarbonyl)-5-(1-((3-(2-((tert-butoxycarbonyl)amino)acetamido)-5,7-dimethyladamantan-1-yl)methyl)-5-methyl-1H-pyrazol-4-yl)pyridin-2-yl)-1,2,3,4-tetrahydroisoquinoline-8-carboxylate

The title compound was prepared using the procedure in Example 1.2.2, substituting Example 1.1.6 with Example 1.89.5. MS (ESI) m/e 797.4 (M+H)⁺.

### 1.89.9 2-(6-(tert-butoxycarbonyl)-5-(1-((3-(2-((tert-butoxycarbonyl)amino)acetamido)-5,7-dimethyladamantan-1-yl)methyl)-5-methyl-1H-pyrazol-4-yl)pyridin-2-yl)-1,2,3,4-tetrahydroisoquinoline-8-carboxylic Acid

The title compound was prepared using the procedure in Example 1.2.5, substituting Example 1.2.4 with Example 1.89.8. MS (ESI) m/e 783.4 (M+H)⁺.

### 1.89.10 tert-butyl 6-(8-(benzo[d]thiazol-2-ylcarbamoyl)-3,4-dihydroisoquinolin-2(1H)-yl)-3-(1-((3-(2-((tert-butoxycarbonyl)amino)acetamido)-5,7-dimethyladamantan-1-yl)methyl)-5-methyl-1H-pyrazol-4-yl)picolinate

The title compound was prepared using the procedure in Example 1.2.6, substituting Example 1.2.5 with Example 1.89.9. MS (ESI) m/e 915.3 (M+H)⁺.

### 1.89.11 3-(1-{[3-(2-aminoacetamido)-5,7-dimethyltricyclo[3.3.1.1^{3,7}]decan-1-yl]methyl}-5-methyl-1H-pyrazol-4-yl)-6-{8-[(1,3-benzothiazol-2-yl)carbamoyl]-3,4-dihydroisoquinolin-2(1H)-yl}pyridine-2-carboxylic Acid

The title compound was prepared using the procedure in Example 1.2.9, substituting Example 1.2.8 with Example 1.89.10. ¹H NMR (400 MHz, dimethyl sulfoxide-d₆) δ 12.82 (s, 1H), 8.00 (dd, 1H), 7.90-7.79 (m, 4H), 7.76 (d, 1H), 7.59 (dd, 1H), 7.49-7.38 (m, 3H), 7.37-7.29 (m, 2H), 7.25 (s, 1H), 6.92 (d, 1H), 4.92 (s, 2H), 3.85 (t, 2H), 3.77 (s, 2H), 3.40 (q, 2H), 2.98 (t, 2H), 2.07 (s, 3H), 1.63 (s, 2H), 1.57-1.38 (m, 4H), 1.15-0.93 (m, 6H), 0.80 (s, 6H). MS (ESI) m/e 759.2 (M+H)⁺.

### 1.89.12 6-{8-[(1,3-benzothiazol-2-yl)carbamoyl]-3,4-dihydroisoquinolin-2(1H)-yl}-3-{1-[(3,5-dimethyl-7-{2-[(2-sulfoethyl)amino]acetamido}tricyclo[3.3.1.1^{3,7}]decan-1-yl)methyl]-5-methyl-1H-pyrazol-4-yl}pyridine-2-carboxylic Acid

To a solution of Example 1.89.11 (102 mg) in N,N-dimethylformamide (6 mL) was added 4-((tert-butyldiphenylsilyl)oxy)-2,2-dimethylbutyl ethenesulfonate (60 mg), and the mixture was stirred at room temperature over a weekend. The mixture was diluted with ethyl acetate (300 mL), washed with water and brine, and dried over anhydrous sodium sulfate. Filtration and evaporation of the solvent gave a residue that was dissolved in dichloromethane/trifluoroacetic acid (10 mL, 1:1) and stirred at room temperature overnight. The solvents were evaporated, and the residue was purified by reverse-phase HPLC on a Gilson system (C18 column), eluting with 20-80% acetonitrile in water containing 0.1% trifluoroacetic acid, to give the title compound. ¹H NMR (501 MHz, dimethyl sulfoxide-d₆) δ 12.83 (s, 1H), 8.57 (s, 2H), 8.02 (d, 1H), 7.95 (s, 1H), 7.77 (d, 1H), 7.60 (d, 1H), 7.52-7.37 (m, 3H), 7.39-7.29 (m, 2H), 7.26 (s, 1H), 6.94 (d, 1H), 4.94 (s, 2H), 3.87 (t, 2H), 3.79 (s, 2H), 3.16 (q, 2H), 2.99 (t, 2H), 2.77 (t, 2H), 2.08 (s, 3H), 1.64 (s, 2H), 1.55 (d, 2H), 1.45 (d, 2H), 1.21-0.95 (m, 6H), 0.82 (s, 6H). MS (ESI) m/e 867.2 (M+H)⁺.

### 1.90 Synthesis of 6-[8-(1,3-benzothiazol-2-ylcarbamoyl)-3,4-dihydroisoquinolin-2(1H)-yl]-3-(1-{[3,5-dimethyl-7-({2-[(2-sulfoethyl)amino]ethyl}sulfanyl)tricyclo[3.3.1.1^{3,7}]dec-1-yl]methyl}-5-methyl-1H-pyrazol-4-yl)pyridine-2-carboxylic Acid

### 1.90.1 3-((1H-pyrazol-1-yl)methyl)-5,7-dimethyladamantane-1-thiol

A mixture of Example 1.1.3 (2.8 g) and thiourea (15.82 g) in 33% (w/w) HBr in acetic acid (50 mL) was stirred at 110° C. for 16 hours and was concentrated under reduced pressure to give a residue. The residue was dissolved in 20% ethanol in water (v/v: 200 mL), and sodium hydroxide (19.06 g) was added. The resulting solution was stirred at room temperature for 16 hours and was concentrated. The residue was dissolved in water (60 mL), and acidified with 6 N aqueous HCl to pH 5-pH 6. The mixture was extracted with ethyl acetate (200 mL×2). The combined organic layers were washed with brine, dried over MgSO₄, filtered and concentrated to give the title compound. MS (ESI) m/e 319.1 (M+H)⁺.

### 1.90.2 2-((−3-((1H-pyrazol-1-yl)methyl)-5,7-dimethyladamantan-1-yl)thio)ethanol

To a solution of Example 1.90.1 (3.3 g) in ethanol (120 mL) was added sodium ethoxide (2.437 g). The mixture was stirred for 10 minutes, and 2-chloroethanol (1.80 mL) was added dropwise. The mixture was stirred at room temperature for 6 hours and was neutralized with 1 N aqueous HCl to pH 7. The mixture was concentrated, and the residue was extracted with ethyl acetate (200 mL×2). The combined organic layers were washed with brine, dried over MgSO₄, filtered and concentrated. The residue was purified by column chromatography on silica gel, eluting with petroleum ether/ethyl acetate from 6/1 to 2/1, to give the title compound. MS (ESI) m/e 321.2 (M+H)⁺.

### 1.90.3 2-((−3,5-dimethyl-7-((5-methyl-1H-pyrazol-1-yl)methyl)adamantan-1-yl)thio)ethanol

To a solution of Example 1.90.2 (2.3 g) in tetrahydrofuran (60 mL) was added n-butyllithium (14.35 mL, 2M in hexane) at −20° C. dropwise under nitrogen. The mixture was stirred at this temperature for 2 hours. Methyl iodide (4.49 mL) was added to the resulting mixture at −20° C., and the mixture was stirred at −20° C. for 2 hours. The reaction was quenched by the dropwise addition of saturated aqueous NH₄Cl solution at −20° C. The resulting mixture was stirred for 10 minutes and acidified with 1 N aqueous HCl to pH 5. The mixture was extracted with ethyl acetate twice. The combined organic layers were washed with brine, dried over MgSO₄, filtered and concentrated to give the title compound. MS (ESI) m/e 335.3 (M+H)⁺.

### 1.90.4 2-((−3-((4-iodo-5-methyl-1H-pyrazol-1-yl)methyl)-5,7-dimethyladamantan-1-yl)thio)ethanol

To a solution of Example 1.90.3 (3.65 g) in N,N-dimethylformamide (90 mL) was added N-iodosuccinimide (3.68 g). The mixture was stirred at room temperature for 16 hours. The reaction was quenched by the addition of ice-water (8 mL) and saturated aqueous NaS₂O₃ solution (8 mL). The mixture was stirred for an additional 10 minutes and was extracted with ethyl acetate (30 mL×2). The combined organic layers were washed with brine, dried over MgSO₄, filtered and concentrated under reduced pressure. The residue was purified by silica gel chromatography, eluting with petroleum ether/ethyl acetate (6/1 to 3/1), to give the title compound. MS (ESI) m/e 461.2 (M+H)⁺.

### 1.90.5 di-tert-butyl [2-({3-[(4-iodo-5-methyl-1H-pyrazol-1-yl)methyl]-5,7-dimethyltricyclo[3.3.1.1^{3,7}]decan-1-yl}sulfanyl)ethyl]-2-imidodicarbonate

To a cold solution (0° C. bath) of Example 1.90.4 (3 g) in dichloromethane (100 mL) was added triethylamine (1.181 mL) and mesyl chloride (0.559 mL). The mixture was stirred at room temperature for 4 hours, and the reaction was quenched by the addition of ice-water (30 mL). The mixture was stirred for an additional 10 minutes and was extracted with dichloromethane (50 mL×2). The combined organic layers were washed with brine, dried over MgSO₄, filtered and concentrated under reduced pressure. The residue was dissolved in acetonitrile (100 mL) and NH(Boc)₂ (1.695 g) and Cs₂CO₃ (4.24 g) were added. The mixture was stirred at 85° C. for 16 hours, and the reaction was quenched by the addition of water (20 mL). The mixture was stirred for 10 minutes and was extracted with ethyl acetate (40 mL×2). The combined organic layers were washed with brine, dried over MgSO₄, filtered and concentrated. The residue was purified by silica gel chromatography, eluting with petroleum ether/ethyl acetate from 10/1 to 6/1, to give the title compound. MS (ESI) m/e 660.1 (M+H)⁺.

### 1.90.6 methyl 2-[5-(1-{[3-({2-[bis(tert-butoxycarbonyl)amino]ethyl}sulfanyl)-5,7-dimethyltricyclo[3.3.1.1^{3,7}]decan-1-yl]methyl}-5-methyl-1H-pyrazol-4-yl)-6-(tert-butoxycarbonyl)pyridin-2-yl]-1,2,3,4-tetrahydroisoquinoline-8-carboxylate

The title compound was prepared using the procedure in Example 1.2.2, replacing Example 1.1.6 with Example 1.90.5. MS (ESI) m/e 900.2 (M+H)⁺.

### 190.7A 2-(6-(tert-butoxycarbonyl)-5-(1-((3-((2-((tert-butoxycarbonyl)amino)ethyl)thio)-5,7-dimethyladamantan-1-yl)methyl)-5-methyl-1H-pyrazol-4-yl)pyridin-2-yl)-1,2,3,4-tetrahydroisoquinoline-8-carboxylic Acid

The title compound was prepared as described in Example 1.2.5, replacing Example 1.2.4 with Example 1.90.6. MS (ESI) m/e 786.2 (M+H)⁺.

### 190.7B tert-butyl 6-(8-(benzo[d]thiazol-2-ylcarbamoyl)-3,4-dihydroisoquinolin-2(1H)-yl)-3-(1-((3-((2-((tert-butoxycarbonyl)amino)ethyl)thio)-5,7-dimethyladamantan-1-yl)methyl)-5-methyl-1H-pyrazol-4-yl)picolinate

The title compound was prepared as described in Example 1.2.6, replacing Example 1.2.5 with Example 1.90.7A. MS (ESI) m/e 918.8 (M+H)⁺.

### 1.90.8 tert-butyl 3-(1-((3-((2-aminoethyl)thio)-5,7-dimethyladamantan-1-yl)methyl)-5-methyl-1H-pyrazol-4-yl)-6-(8-(benzo[d]thiazol-2-ylcarbamoyl)-3,4-dihydroisoquinolin-2(1H)-yl)picolinate

To a solution of Example 1.90.7B (510 mg) in dichloromethane (5 mL) was added trifluoroacetic acid (5 mL,) and the reaction was stirred at room temperature for 30 minutes. The reaction was quenched by the addition of saturated aqueous sodium bicarbonate solution and extracted with dichloromethane thrice. The combined organics were dried with anhydrous sodium sulfate, filtered and concentrated under reduced pressure. The residue was purified by reverse-phase HPLC on a Gilson system (C18 column), eluting with 20-80% acetonitrile in water containing 0.1% trifluoroacetic acid, to give the title product. MS (ESI) m/e 818.1 (M+H)⁺.

### 1.90.9 3-(1-((3-((2-aminoethyl)thio)-5,7-dimethyladamantan-1-yl)methyl)-5-methyl-1H-pyrazol-4-yl)-6-(8-(benzo[d]thiazol-2-ylcarbamoyl)-3,4-dihydroisoquinolin-2(1H)-yl)picolinic Acid

Example 1.90.9 was isolated during the preparation of Example 1.90.8. MS (ESI) 762.2 (M+H)⁺.

### 1.90.10 tert-butyl 6-(8-(benzo[d]thiazol-2-ylcarbamoyl)-3,4-dihydroisoquinolin-2(1H)-yl)-3-(1-((3-((2-((2-((4-((tert-butyldiphenylsilyl)oxy)-2,2-dimethylbutoxy)sulfonyl)ethyl)amino)ethyl)thio)-5,7-dimethyladamantan-1-yl)methyl)-5-methyl-1H-pyrazol-4-yl)picolinate

Example 1.90.8 (235 mg) and 4-((tert-butyldiphenylsilyl)oxy)-2,2-dimethylbutyl ethenesulfonate (150 mg) were dissolved in dichloromethane (1 mL), N,N-diisopropylethylamine (140 μL) was added, and the mixture was stirred at room temperature for six days. The reaction was directly purified by silica gel chromatography, eluting with a gradient of 0.5-3.0% methanol in dichloromethane, to give the title compound.

### 1.90.11 6-(8-(benzo[d]thiazol-2-ylcarbamoyl)-3,4-dihydroisoquinolin-2(1H)-yl)-3-(1-((3,5-dimethyl-7-((2-((2-sulfoethyl)amino)ethyl)thio)adamantan-1-yl)methyl)-5-methyl-1H-pyrazol-4-yl)picolinic Acid

The title compound was prepared by substituting Example 1.90.10 for Example 1.2.8 in Example 1.2.9. ¹H NMR (500 MHz, dimethyl sulfoxide-d₆) δ ppm 8.39 (br s, 2H), 8.03 (d, 1H), 7.79 (d, 1H), 7.62 (d, 1H), 7.51 (d, 1H), 7.47 (ddd, 1H), 7.43 (d, 1H), 7.37 (d, 1H), 7.35 (ddd, 1H), 7.30 (s, 1H), 6.96 (d, 1H), 4.96 (s, 2H), 3.89 (t, 2H), 3.81 (s, 2H), 3.22 (m, 2H), 3.06 (br m, 2H), 3.01 (t, 2H), 2.79 (t, 2H), 2.74 (m, 2H), 2.10 (s, 3H), 1.51 (s, 2H), 1.37 (m, 4H), 1.15 (m, 4H), 1.05 (m, 2H), 0.83 (s, 6H). MS (ESI) m/e 870.1 (M+H)⁺.

### 1.91 Synthesis of 6-{8-[(1,3-benzothiazol-2-yl)carbamoyl]-3,4-dihydroisoquinolin-2(1H)-yl}-3-{1-[(3,5-dimethyl-7-{3-[(2-sulfoethyl)amino]propyl}tricyclo[3.3.1.1^{3,7}]decan-1-yl)methyl]-5-methyl-1H-pyrazol-4-yl}pyridine-2-carboxylic Acid

### 1.91.1 1-((3-allyl-5,7-dimethyladamantan-1-yl)methyl)-1H-pyrazole

To a solution of Example 1.1.3 (0.825 g, 2.55 mmol) in toluene (5 mL) was added N, N′-azoisobutyronitrile (AIBN, 0.419 g, 2.55 mmol) and allyltributylstannane (2.039 ml, 6.38 mmol). The mixture was purged with N₂ stream for 15 minutes, heated at 80° C. for 8 hours and concentrated. The residue was purified by flash chromatography, eluting with 5% ethyl acetate in petroleum ether to provide the title compound. MS (ESI) m/e 285.2 (M+H)⁺.

### 1.91.2 1-((3-allyl-5,7-dimethyladamantan-1-yl)methyl)-5-methyl-1H-pyrazole

To a solution of Example 1.91.1 (200 mg, 0.703 mmol) in tetrahydrofuran (5 ml) at −78° C. under N₂ was added n-butyllithium (2.81 mL, 7.03 mmol). The mixture was stirred for 2 hours while the temperature increased to −20° C. and then it was stirred at −20° C. for 1 hour. Iodomethane (0.659 ml, 10.55 mmol) was added and the resulting mixture was stirred for 0.5 hours at −20° C. The reaction was quenched with saturated NH₄Cl and extracted with ethyl acetate twice. The combined organic layer was washed with brine and concentrated to give the title compound. MS (ESI) m/e 299.2 (M+H)⁺.

### 1.91.3 3-(3,5-dimethyl-7-((5-methyl-1H-pyrazol-1-yl)methyl)adamantan-1-yl)propan-1-ol

Under nitrogen atmosphere, a solution of Example 1.91.2 (2.175 g, 7.29 mmol) in anhydrous tetrahydrofuran (42.5 mL) was cooled to 0° C. BH₃·THF (15.30 mL, 15.30 mmol) was added dropwise. The reaction mixture was stirred at room temperature for 2 hours and cooled to 0° C. To the reaction mixture was added 10 N aqueous NaOH (5.03 mL, 50.3 mmol) dropwise, followed by 30 percent H₂O₂ (16.52 mL, 146 mmol) water solution. The resulting mixture was warmed to room temperature and stirred for 90 minutes. The reaction was quenched with 10 percent hydrochloric acid (35 mL). The organic layer was separated and the aqueous layer was extracted with ethyl acetate (2×60 mL). The combined organic layers were washed with brine (3×60 mL) and cooled in an ice bath. A saturated aqueous solution of sodium sulfite (15 mL) was carefully added and the mixture was stirred for a few minutes. The organic layer was dried over sodium sulfate, filtered, and concentrated in vacuo. The residue was purified by flash chromatography, eluting with petroleum ether/ethyl acetate (3:1 to 1:1) to provide the title compound. MS (ESI) m/e 317.3 (M+H)⁺.

### 1.91.4 3-(3-((4-iodo-5-methyl-1H-pyrazol-1-yl)methyl)-5,7-dimethyladamantan-1-yl)propan-1-ol

A mixture of Example 1.91.3 (1.19 g, 3.76 mmol) and 1-iodopyrrolidine-2, 5-dione (1.015 g, 4.51 mmol) in N,N-dimethylformamide (7.5 mL) was stirred for 16 hours at room temperature. The reaction was quenched with saturated Na₂SO₃. The mixture was diluted with ethyl acetate and washed with saturated Na₂SO₃, saturated Na₂CO₃, water and brine. The organic layer was dried over anhydrous Na₂SO₄, filtered, and concentrated. The residue was purified by flash chromatography, eluting with petroleum ether/ethyl acetate (3:1 to 1:1) to provide the title compound. MS (ESI) m/e 443.1 (M+H)⁺.

### 1.91.5 3-(3-((4-iodo-5-methyl-1H-pyrazol-1-yl)methyl)-5,7-dimethyladamantan-1-yl)propyl methanesulfonate

To a solution of Example 1.91.4 (1.55 g, 3.50 mmol) in CH₂Cl₂ (20 mL) at 0° C. were added (CH₃CH₂)₃N (0.693 mL, 4.98 mmol) and mesyl chloride (0.374 mL, 4.80 mmol) slowly. The mixture was stirred for 3.5 hours at 20° C. and diluted with CH₂Cl₂, washed with saturated NH₄Cl, NaHCO₃ and brine. The organic layer was dried over Na₂SO₄, filtered, and concentrated to provide the title compound. MS (ESI) m/e 521.1 (M+H)⁺.

### 1.91.6 di-tert-butyl (3-{3-[(4-iodo-5-methyl-1H-pyrazol-1-yl)methyl]-5,7-dimethyltricyclo[3.3.1.1^{3,7}]decan-1-yl}propyl)-2-imidodicarbonate

To a solution of Example 1.91.5 (1.92 g, 3.69 mmol) in CH₃CN (40 ml) at 20° C. was added di-tert-butyl imidodicarbonate (0.962 g, 4.43 mmol) and Cs₂CO₃ (2.404 g, 7.38 mmol). The mixture was stirred for 16 hours at 80° C. and was diluted with ethyl acetate, and was washed with water and brine. The organic layer was dried over Na₂SO₄, filtered, and concentrated. The residue was purified by flash chromatography, eluting with petroleum ether/ethyl acetate (10:1) to provide the title compound. MS (ESI) m/e 642.3 (M+H)⁺.

### 1.91.7 methyl 2-[5-{1-[(3-{3-[bis(tert-butoxycarbonyl)amino]propyl}-5,7-dimethyltricyclo[3.3.1.1^{3,7}]decan-1-yl)methyl]-5-methyl-1H-pyrazol-4-yl}-6-(tert-butoxycarbonyl)pyridin-2-yl]-1,2,3,4-tetrahydroisoquinoline-8-carboxylate

The title compound was prepared using the procedure in Example 1.2.2, replacing Example 1.1.6 with Example 1.91.6. MS (ESI) m/e 882.2 (M+H)⁺.

### 1.91.8 2-[6-(tert-butoxycarbonyl)-5-{1-[(3-{3-[(tert-butoxycarbonyl)amino]propyl}-5,7-dimethyltricyclo[3.3.1.1^{3,7}]decan-1-yl)methyl]-5-methyl-1H-pyrazol-4-yl}pyridin-2-yl]-1,2,3,4-tetrahydroisoquinoline-8-carboxylic Acid

The title compound was prepared using the procedure in Example 1.2.5, replacing Example 1.2.4 with Example 1.91.7. MS (ESI) m/e 768.4 (M+H)⁺.

### 1.91.9 tert-butyl 6-(8-(benzo[d]thiazol-2-ylcarbamoyl)-3,4-dihydroisoquinolin-2(1H)-yl)-3-(1-((3-(3-((tert-butoxycarbonyl)amino)propyl)-5,7-dimethyladamantan-1-yl)methyl)-5-methyl-1H-pyrazol-4-yl)picolinate

The title compound was prepared using the procedure in Example 1.2.6, replacing Example 1.2.5 with Example 1.91.8. MS (ESI) m/e 901.1 (M+H)⁺.

### 1.91.10 tert-butyl 3-(1-((3-(3-aminopropyl)-5,7-dimethyladamantan-1-yl)methyl)-5-methyl-1H-pyrazol-4-yl)-6-(8-(benzo[d]thiazol-2-ylcarbamoyl)-3,4-dihydroisoquinolin-2(1H)-yl)picolinate

To a solution of Example 1.91.9 (500 mg) in dichloromethane (5 mL) was added trifluoroacetic acid (5 mL) and the reaction was stirred at room temperature for 30 minutes. The reaction was quenched by the addition of saturated aqueous sodium bicarbonate solution and extracted with dichloromethane thrice. The combined organics were dried with anhydrous sodium sulfate, filtered and concentrated under reduced pressure. The residue was purified by reverse-phase HPLC on a Gilson system (C18 column), eluting with 20-80% acetonitrile in water containing 0.1% trifluoroacetic acid, to give the title product.

### 1.91.11 3-(1-((3-(3-aminopropyl)-5,7-dimethyladamantan-1-yl)methyl)-5-methyl-1H-pyrazol-4-yl)-6-(8-(benzo[d]thiazol-2-ylcarbamoyl)-3,4-dihydroisoquinolin-2(1H)-yl)picolinic Acid

To a solution of Example 1.91.9 (350 mg) in dichloromethane (5 mL) was added trifluoroacetic acid (5 mL). The mixture was stirred overnight. The mixture was concentrated and the residue was purified by reverse phase HPLC using a Gilson system, eluting with 20-80% acetonitrile in water containing 0.1% v/v trifluoroacetic acid, to provide the title compound. ¹H NMR (500 MHz, DMSO-d₆) δ ppm 12.86 (s, 1H), 8.03 (d, 1H), 7.79 (d, 1H), 7.62 (d, 4H), 7.47 (dt, 3H), 7.36 (q, 2H), 7.27 (s, 1H), 6.95 (d, 1H), 4.95 (s, 2H), 3.77 (s, 2H), 3.01 (t, 2H), 2.72 (q, 2H), 2.09 (s, 3H), 1.45 (t, 2H), 1.18-1.05 (m, 9H), 1.00 (d, 6H), 0.80 (s, 6H). MS (ESI) m/e 744.2 (M+H)⁺.

### 1.91.12 tert-butyl 6-(8-(benzo[d]thiazol-2-ylcarbamoyl)-3,4-dihydroisoquinolin-2(1H)-yl)-3-(1-((3-(3-((2-((4-((tert-butyldiphenylsilyl)oxy)-2,2-dimethylbutoxy)sulfonyl)ethyl)amino)propyl)-5,7-dimethyladamantan-1-yl)methyl)-5-methyl-1H-pyrazol-4-yl)picolinate

The title compound was prepared using the procedure in Example 1.2.8, replacing Example 1.2.7 with Example 1.91.10.

### 1.91.13 6-{8-[(1,3-benzothiazol-2-yl)carbamoyl]-3,4-dihydroisoquinolin-2(1H)-yl}-3-{1-[(3,5-dimethyl-7-{3-[(2-sulfoethyl)amino]propyl}tricyclo[3.3.1.1^{3,7}]decan-1-yl)methyl]-5-methyl-1H-pyrazol-4-yl}pyridine-2-carboxylic Acid

The title compound was prepared using the procedure in Example 1.2.9, replacing Example 1.2.8 with Example 1.91.12. ¹H NMR (501 MHz, DMSO-d₆) δ 12.85 (s, 1H), 8.02 (dd, 1H), 7.77 (d, 1H), 7.60 (d, 1H), 7.54-7.39 (m, 3H), 7.38-7.31 (m, 2H), 7.26 (s, 1H), 6.94 (d, 1H), 4.94 (s, 2H), 3.87 (t, 2H), 3.15 (p, 2H), 3.00 (t, 2H), 2.86 (dq, 2H), 2.76 (t, 2H), 2.08 (s, 3H), 1.47 (td, 2H), 1.08 (d, 9H), 0.99 (d, 7H), 0.79 (s, 7H). MS (ESI) m/e 852.2 (M+H)⁺.

### Example 1. Synthesis of Exemplary Synthons

This example provides synthetic methods for exemplary synthons useful to make ADCs.

### 1.1. Synthesis of N-[6-(2,5-dioxo-2,5-dihydro-1H-pyrrol-1-yl)hexanoyl]-L-valyl-N-{4-[({[2-({3-[(4-{6-[8-(1,3-benzothiazol-2-ylcarbamoyl)-3,4-dihydroisoquinolin-2(1H)-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](2-sulfoethyl)carbamoyl}oxy)methyl]phenyl}-N⁵-carbamoyl-L-ornithinamide (Synthon CZ)

Example 1.2.9 (100 mg) and 4-((S)-2-((S)-2-(6-(2,5-dioxo-2,5-dihydro-1H-pyrrol-1-yl)hexanamido)-3-methylbutanamido)-5-ureidopentanamido)benzyl (4-nitrophenyl) carbonate (purchased from Synchem, 114 mg) in N,N-dimethylformamide (7 mL) was cooled in an water-ice bath, and N,N-diisopropylethylamine (0.15 mL) was added. The mixture was stirred at 0° C. for 30 minutes and then at room temperature overnight. The reaction was purified by a reverse phase HPLC using a Gilson system, eluting with 20-60% acetonitrile in water containing 0.1% v/v trifluoroacetic acid, to provide the title compound. ¹H NMR (400 MHz, dimethyl sulfoxide-d₆) δ ppm 12.85 (s, 1H), 9.99 (s, 1H), 8.04 (t, 2H), 7.75-7.82 (m, 2H), 7.40-7.63 (m, 6H), 7.32-7.39 (m, 2H), 7.24-7.29 (m, 3H), 6.99 (s, 2H), 6.95 (d, 1H), 6.01 (s, 1H), 4.83-5.08 (m, 4H), 4.29-4.48 (m, 1H), 4.19 (t, 1H), 3.84-3.94 (m, 2H), 3.80 (d, 2H), 3.14-3.29 (m, 2H), 2.87-3.06 (m, 4H), 2.57-2.69 (m, 2H), 2.03-2.24 (m, 5H), 1.89-2.02 (m, 1H), 1.53-1.78 (m, 2H), 1.26-1.53 (m, 8H), 0.89-1.27 (m, 12H), 0.75-0.88 (m, 12H). MS (ESI) m/e 1452.2 (M+H)⁺.

### 1.2. Synthesis of N-[6-(2,5-dioxo-2,5-dihydro-1H-pyrrol-1-yl)hexanoyl]-L-valyl-N-{4-[({[2-({3-[(4-{6-[8-(1,3-benzothiazol-2-ylcarbamoyl)-3,4-dihydroisoquinolin-2(1H)-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](3-sulfopropyl)carbamoyl}oxy)methyl]phenyl}-N⁵-carbamoyl-L-ornithinamide (Synthon DH)

The title compound was prepared as described in Example 2.1, replacing Example 1.2.9 with Example 1.6.2. ¹H NMR (400 MHz, dimethyl sulfoxide-d₆) δ ppm 12.83 (s, 1H), 9.98 (s, 1H), 8.04 (t, 2H), 7.75-7.81 (m, 2H), 7.54-7.64 (m, 3H), 7.40-7.54 (m, 3H), 7.32-7.39 (m, 2H), 7.24-7.31 (m, 3H), 6.93-7.01 (m, 3H), 4.86-5.03 (m, 4H), 4.32-4.48 (m, 2H), 4.13-4.26 (m, 2H), 3.31-3.45 (m, 4H), 3.24 (d, 4H), 2.88-3.07 (m, 4H), 2.30-2.39 (m, 2H), 2.04-2.24 (m, 5H), 1.86-2.03 (m, 1H), 0.89-1.82 (m, 27H), 0.74-0.88 (m, 13H). MS (ESI) m/e 1466.3 (M+H)⁺.

### 1.3. This Paragraph was Intentionally Left Blank

### 1.4. Synthesis of N-[6-(2,5-dioxo-2,5-dihydro-1H-pyrrol-1-yl)hexanoyl]-L-valyl-N-[4-({[{2-[2-({3-[(4-{6-[8-(1,3-benzothiazol-2-ylcarbamoyl)-3,4-dihydroisoquinolin-2(1H)-yl]-2-carboxypyridin-3-yl}-5-methyl-1H-pyrazol-1-yl)methyl]-5,7-dimethyltricyclo[3.3.1.1^{3,7}]dec-1-yl}oxy)ethoxy]ethyl}(2-sulfoethyl)carbamoyl]oxy}methyl)phenyl]-N⁵-carbamoyl-L-ornithinamide (Synthon EP)

The title compound was prepared as described in Example 2.1, replacing Example 1.2.9 with Example 1.11.4. ¹H NMR (500 MHz, dimethyl sulfoxide-d₆) δ ppm 12.85 (s, 1H), 10.00 (s, 1H), 8.01-8.10 (m, 2H), 7.79 (dd, 2H), 7.55-7.65 (m, 3H), 7.41-7.53 (m, 3H), 7.32-7.38 (m, 2H), 7.25-7.30 (m, 3H), 6.97-7.02 (m, 2H), 6.96 (d, 1H), 6.03 (s, 1H), 4.90-5.03 (m, 4H), 4.31-4.46 (m, 1H), 4.20 (s, 1H), 3.88 (t, 2H), 3.82 (s, 2H), 2.97-3.06 (m, 2H), 2.88-2.98 (m, 1H), 2.58-2.68 (m, 2H), 2.05-2.22 (m, 5H), 1.92-2.02 (m, 1H), 0.89-1.75 (m, 23H), 0.77-0.87 (m, 12H). MS (ESI) m/e 1496.3 (M+H)⁺.

### 1.5. Synthesis of methyl 6-[4-(3-{[2-({3-[(4-{6-[8-(1,3-benzothiazol-2-ylcarbamoyl)-3,4-dihydroisoquinolin-2(1H)-yl]-2-carboxypyridin-3-yl)-5-methyl-1H-pyrazol-1-yl)methyl]-5,7-dimethyltricyclo[3.3.1.1^{3,7}]dec-1-yl}oxy)ethyl]({[4-((N-[6-(2,5-dioxo-2,5-dihydro-1H-pyrrol-1-yl)hexanoyl]-L-valyl-N⁵-carbamoyl-L-ornithyl}amino)benzyl]oxy}carbonyl)amino}propyl)-1H-1,2,3-triazol-1-yl]-6-deoxy-beta-L-glucopyranoside (Synthon EF)

### 1.5.1. pent-4-ynal

To a solution of oxalyl chloride (9.12 mL) dissolved in dichloromethane (200 mL) at −78° C. was added dimethyl sulfoxide (14.8 mL) dissolved in dichloromethane (40 mL) over 20 minutes. After the solution was stirred for an additional 30 minutes, 4-pentynol (8.0 g) dissolved in dichloromethane (80 mL) was added over 10 minutes. The reaction mixture was stirred at −78° C. for an additional 60 minutes. Triethylamine (66.2 mL) was added at −78° C., and the reaction mixture was stirred for 60 minutes and then allowed to warm to 10° C. over an additional hour. Water (200 mL) was added, and the two layers were separated. The aqueous layer was acidified with 1% aqueous HCl and then back-extracted with dichloromethane (3×100 mL). The combined organic layers were washed with 1% aqueous HCl, and aqueous NaHCO₃. The aqueous extracts were back-extracted with dichloromethane (2×100 mL), and the combined organic extracts were washed with brine and dried over sodium sulfate. After filtration, the solvent was removed by rotary evaporation (30° C. water bath) to provide the title compound.

### 1.5.2. 6-(8-(benzo[d]thiazol-2-ylcarbamoyl)-3,4-dihydroisoquinolin-2(1H)-yl)-3-(1-((3,5-dimethyl-7-(2-(pent-4-yn-1-ylamino)ethoxy)adamantan-1-yl)methyl)-5-methyl-1H-pyrazol-4-yl)picolinic Acid

To a solution of Example 1.2.7 (85 mg) in tetrahydrofuran (2 mL) was added pent-4-yanl (8.7 mg), acetic acid (20 mg) and sodium sulfate (300 mg). The mixture was stirred for 1 hour, and sodium triacetoxyborohydride (45 mg) was added to the reaction mixture. The mixture was stirred overnight, then diluted with ethyl acetate (200 mL), washed with water and brine, and dried over sodium sulfate. Filtration and evaporation of the solvent gave a residue, which was dissolved in dimethyl sulfoxide/methanol (1:1, 3 mL). The mixture was purified by reverse phase HPLC on a Gilson system, eluting with 10-85% acetonitrile in 0.1% trifluoroacetic acid in water, to give the title compound. MS (ESI) m/e 812.1 (M+H)⁺.

### 1.5.3. 6-(8-(benzo[d]thiazol-2-ylcarbamoyl)-3,4-dihydroisoquinolin-2(1H)-yl)-3-(1-((3,5-dimethyl-7-(2-((3-(1-(((2S,3R,4R,5S,6S)-3,4,5-trihydroxy-6-methoxytetrahydro-2H-pyran-2-yl)methyl)-1H-1,2,3-triazol-4-yl)propyl)amino)ethoxy)adamantan-1-yl)methyl)-5-methyl-1H-pyrazol-4-yl)picolinic Acid

To a solution of (2S,3S,4R,5S,6S)-2-(azidomethyl)-6-methoxytetrahydro-2H-pyran-3,4,5-triyl triacetate (8.63 mg) in t-butanol (2 mL) and water (1 mL) was added Example 2.5.2 (20 mg), copper(II) sulfate pentahydrate (2.0 mg) and sodium ascorbate (5 mg). The mixture was stirred 20 minutes at 100° C. under microwave conditions (Biotage Initiator). Lithium hydroxide monohydrate (50 mg) was added to the mixture, and it was stirred overnight. The mixture was neutralized with trifluoroacetic acid and purified by reverse phase HPLC (Gilson system), eluting with 10-85% acetonitrile in 0.1% trifluoroacetic acid in water, to provide the title compound. MS (ESI) m/e 1032.2 (M+H)⁺.

### 1.5.4. Methyl 6-[4-(3-{[2-({3-[(4-{6-[8-(1,3-benzothiazol-2-ylcarbamoyl)-3,4-dihydroisoquinolin-2(1H)-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl]({[4-({N-[6-(2,5-dioxo-2,5-dihydro-1H-pyrrol-1-yl)hexanoyl]-L-valyl-N⁵-carbamoyl-L-ornithyl}amino)benzyl]oxy}carbonyl)amino}propyl)-1H-1,2,3-triazol-1-yl]-6-deoxy-beta-L-glucopyranoside

To a solution of 4-((S)-2-((S)-2-(6-(2,5-dioxo-2,5-dihydro-1H-pyrrol-1-yl)hexanamido)-3-methylbutanamido)-5-ureidopentanamido)benzyl 4-nitrophenyl carbonate (7.16 mg) and Example 2.5.3 (10 mg) in N,N-dimethylformamide (2 mL) was added N,N-diisopropylethylamine (0.1 mL). The mixture was stirred overnight, then acidified with trifluoroacetic acid and purified by reverse phase HPLC (Gilson system), eluting with 10-85% acetonitrile in 0.1% trifluoroacetic acid in water, to provide the title compound. ¹H NMR (400 MHz, dimethyl sulfoxide-d₆) δ ppm 9.65 (s, 1H), 7.97 (d, 1H), 7.76 (d, 1H), 7.64-7.72 (m, 2H), 7.53-7.63 (m, 3H), 7.38-7.51 (m, 4H), 7.30-7.37 (m, 2H), 7.22-7.27 (m, 3H), 6.84-6.98 (m, 3H), 4.97 (d, 4H), 4.65 (dd, 1H), 4.50 (d, 1H), 4.36-4.46 (m, 1H), 4.25-4.32 (m, 1H), 4.10-4.20 (m, 1H), 3.85-3.95 (m, 2H), 3.79 (s, 2H), 3.66-3.73 (m, 2H), 2.99-3.03 (m, 7H), 2.57 (t, 3H), 2.12-2.22 (m, 3H), 2.08 (s, 3H), 1.99-2.05 (m, 2H), 1.70-1.88 (m, 4H), 1.39-1.67 (m, 8H), 1.35 (s, 3H), 0.92-1.28 (m, 14H), 0.80-0.88 (m, 16H). MS (ESI) m/e 1629.5 (M+H)⁺.

### 1.6. Synthesis of N-[6-(2,5-dioxo-2,5-dihydro-1H-pyrrol-1-yl)hexanoyl]-L-valyl-N-(4-{[([2-({3-[(4-{6-[8-(1,3-benzothiazol-2-ylcarbamoyl)-3,4-dihydroisoquinolin-2(1H)-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl]{3-[1-(beta-D-glucopyranuronosyl)-1H-1,2,3-triazol-4-yl]propyl}carbamoyl) oxy]methyl}phenyl)-N⁵-carbamoyl-L-ornithinamide (Synthon EG)

### 1.6.1. 6-(8-(benzo[d]thiazol-2-ylcarbamoyl)-3,4-dihydroisoquinolin-2(1H)-yl)-3-(1-((3-(2-((3-(1-((2R,3R,4S,5S,6S)-6-carboxy-3,4,5-trihydroxytetrahydro-2H-pyran-2-yl)-1H-1,2,3-triazol-4-yl)propyl)amino)ethoxy)-5,7-dimethyladamantan-1-yl)methyl)-5-methyl-1H-pyrazol-4-yl)picolinic Acid

To a solution of (2R,3R,4S,5S,6S)-2-azido-6-(methoxycarbonyl)tetrahydro-2H-pyran-3,4,5-triyl triacetate (8.63 mg) in t-butanol (2 mL) and water (1 mL) was added Example 2.5.2 (20 mg), copper(II) sulfate pentahydrate (2.0 mg) and sodium ascorbate (5 mg). The mixture was stirred 20 minutes at 100° C. under microwave conditions (Biotage Initiator). Lithium hydroxide monohydrate (50 mg) was added to the mixture, and it was stirred overnight. The mixture was neutralized with trifluoroacetic acid and purified by reverse phase HPLC (Gilson system) eluting with 10-85% acetonitrile in 0.1% trifluoroacetic acid in water, to provide the title compound. MS (ESI) m/e 1032.1 (M+H)⁺.

### 1.6.2. N-[6-(2,5-dioxo-2,5-dihydro-1H-pyrrol-1-yl)hexanoyl]-L-valyl-N-(4-{[([2-({3-[(4-{6-[8-(1,3-benzothiazol-2-ylcarbamoyl)-3,4-dihydroisoquinolin-2(1H)-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl]{3-[1-(beta-D-glucopyranuronosyl)-1H-1,2,3-triazol-4-yl]propyl}carbamoyl)oxy]methyl}phenyl)-N⁵-carbamoyl-L-ornithinamide

The title compound was prepared by substituting Example 2.6.1 for Example 2.5.3 in Example 2.5.4. ¹H NMR (400 MHz, dimethyl sulfoxide-d₆) δ ppm 9.64 (s, 1H), 7.98 (d, 1H), 7.90 (s, 1H), 7.76 (d, 1H), 7.68 (s, 1H), 7.52-7.62 (m, 3H), 7.20-7.50 (m, 9H), 6.84-6.98 (m, 3H), 5.56 (d, 1H), 4.98 (d, 4H), 4.36-4.49 (m, 2H), 4.11-4.23 (m, 2H), 3.96 (d, 2H), 3.74-3.91 (m, 7H), 3.51-3.58 (m, 5H), 3.35-3.49 (m, 10H), 2.97-3.02 (m, 6H), 2.57-2.66 (m, 3H), 2.12-2.24 (m, 2H), 2.08 (s, 3H), 1.69-2.01 (m, 3H), 1.35-1.65 (m, 9H), 0.93-1.28 (m, 10H), 0.81-0.89 (m, 10H). MS (ESI) m/e 1629.4 (M+H)⁺.

### 1.7. Synthesis of N-[6-(2,5-dioxo-2,5-dihydro-1H-pyrrol-1-yl)hexanoyl]-L-valyl-N-{4-[({[(2R)-1-{[2-({3-[(4-{6-[8-(1,3-benzothiazol-2-ylcarbamoyl)-3,4-dihydroisoquinolin-2(1H)-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](methyl)amino}-1-oxo-3-sulfopropan-2-yl]carbamoyl}oxy)methyl]phenyl}-L-alaninamide (Synthon EH)

To a solution of Example 1.13.8 (0.018 g) and 4-((S)-2-((S)-2-(6-(2,5-dioxo-2,5-dihydro-1H-pyrrol-1-yl)hexanamido)-3-methylbutanamido)propanamido)benzyl (4-nitrophenyl) carbonate (0.015 g, 0.023 mmol) in N,N-dimethylformamide (0.75 mL) was added N,N-diisopropylethylamine (0.015 mL). After stirring overnight, the reaction was diluted with N,N-dimethylformamide (0.75 mL) and water (0.5 mL). The mixture was purified by reverse phase HPLC using a Gilson system, eluting with 10-70% acetonitrile in water containing 0.1% v/v trifluoroacetic acid. The desired fractions were combined and freeze-dried to provide the title compound. ¹H NMR (500 MHz, dimethyl sulfoxide-d₆) δ ppm 12.86 (s, 1H), 9.93 (s, 1H), 8.14 (d, 1H), 8.04 (d, 1H), 7.84-7.76 (m, 2H), 7.61 (d, 1H), 7.57 (d, 2H), 7.53 (dd, 1H), 7.47 (t, 1H), 7.43 (d, 1H), 7.39-7.30 (m, 4H), 7.26 (d, 2H), 6.99 (s, 2H), 6.97 (dd, 1H), 4.96 (s, 2H), 4.90 (t, 2H), 4.75-4.65 (m, 1H), 4.46-4.33 (m, 2H), 4.17 (dd, 2H), 3.66-3.47 (m, 4H), 3.36 (t, 4H), 3.12 (s, 2H), 3.01 (t, 2H), 2.85-2.60 (m, 4H), 2.25-2.05 (m, 5H), 2.05-1.90 (m, 1H), 1.58-0.76 (m, 32H). MS (ESI) m/e 1423.2 (M+H)⁺.

### 1.8. Synthesis of N-[6-(2,5-dioxo-2,5-dihydro-1H-pyrrol-1-yl)hexanoyl]-L-valyl-N-{4-[({[2-({3-[(4-{6-[8-(1,3-benzothiazol-2-ylcarbamoyl)-3,4-dihydroisoquinolin-2(1H)-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl][4-(beta-D-glucopyranosyloxy)benzyl]carbamoyl}oxy)methyl]phenyl}-N⁵-carbamoyl-L-ornithinamide (Synthon ER)

### 1.8.1. 6-(8-(benzo[d]thiazol-2-ylcarbamoyl)-3,4-dihydroisoquinolin-2(1H)-yl)-3-(1-((3,5-dimethyl-7-(2-((4-(((2S,3R,4S,5S,6R)-3,4,5-trihydroxy-6-(hydroxymethyl)tetrahydro-2H-pyran-2-yl)oxy)benzyl)amino)ethoxy)adamantan-1-yl)methyl)-5-methyl-1H-pyrazol-4-yl)picolinic Acid

To a solution of Example 1.2.7 (44.5 mg) in tetrahydrofuran (2 mL) and acetic acid (0.2 mL) was added 4-(((2S,3R,4S,5S,6R)-3,4,5-trihydroxy-6-(hydroxymethyl)tetrahydro-2H-pyran-2-yl)oxy)benzaldehyde (17 mg) and MgSO₄ (300 mg). The mixture was stirred for 1 hour before the addition of sodium cyanoborohydride on resin (300 mg). The mixture was stirred overnight. The mixture was filtered, and the solvent was evaporated. The residue was dissolved in dimethyl sulfoxide/methanol (1:1, 4 mL) and purified by reverse phase HPLC (Gilson system), eluting with 10-85% acetonitrile in 0.1% trifluoroacetic acid in water, to give the title compound. MS (ESI) m/e 1015.2 (M+H)⁺.

### 1.8.2. N-[6-(2,5-dioxo-2,5-dihydro-1H-pyrrol-1-yl)hexanoyl]-L-valyl-N-{4-[({[2-({3-[(4-{6-[8-(1,3-benzothiazol-2-ylcarbamoyl)-3,4-dihydroisoquinolin-2(1H)-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl][4-(beta-D-glucopyranosyloxy)benzyl]carbamoyl}oxy)methyl]phenyl}-N⁵-carbamoyl-L-ornithinamide

The title compound was prepared by substituting Example 2.8.1 for Example 2.5.3 in Example 2.5.4. ¹H NMR (400 MHz, dimethyl sulfoxide-d₆) δ ppm 12.87 (s, 1H), 10.00 (s, 1H), 7.96-8.14 (m, 2H), 7.79 (d, 2H), 7.55-7.68 (m, 3H), 7.09-7.52 (m, 11H), 6.91-7.01 (m, 5H), 5.09 (d, 1H), 4.95 (dd, 4H), 4.35-4.47 (m, 4H), 4.14-4.23 (m, 3H), 3.86-3.94 (m, 6H), 3.31-3.46 (m, 8H), 3.16-3.25 (m, 3H), 2.90-3.04 (m, 4H), 2.59 (s, 1H), 1.88-2.24 (m, 6H), 0.88-1.75 (m, 24H), 0.76-0.90 (m, 12H). MS (ESI) m/e 1613.7 (M+H)⁺.

### 1.9. Synthesis of N-[6-(2,5-dioxo-2,5-dihydro-1H-pyrrol-1-yl)hexanoyl]-L-valyl-N-{4-[({[4-(beta-D-allopyranosyloxy)benzyl][2-({3-[(4-{6-[8-(1,3-benzothiazol-2-ylcarbamoyl)-3,4-dihydroisoquinolin-2(1H)-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl]carbamoyl}oxy)methyl]phenyl}-N⁵-carbamoyl-L-ornithinamide (Synthon ES)

### 1.9.1. 6-(8-(benzo[d]thiazol-2-ylcarbamoyl)-3,4-dihydroisoquinolin-2(1H)-yl)-3-(1-((3,5-dimethyl-7-(2-((4-(((2S,3R,4R,5S,6R)-3,4,5-trihydroxy-6-(hydroxymethyl)tetrahydro-2H-pyran-2-yl)oxy)benzyl)amino)ethoxy)adamantan-1-yl)methyl)-5-methyl-1H-pyrazol-4-yl)picolinic Acid

To a solution of Example 1.2.7 (44.5 mg) in tetrahydrofuran (2 mL) and acetic acid (0.2 mL) was added 4-(((2S,3R,4R,5S,6R)-3,4,5-trihydroxy-6-(hydroxymethyl)tetrahydro-2H-pyran-2-yl)oxy)benzaldehyde (17 mg) and MgSO₄ (300 mg). The mixture was stirred for 1 hour before the addition of sodium cyanoborohydride on resin (300 mg). The mixture was stirred overnight. The mixture was filtered, and the solvent was evaporated. The residue was dissolved in dimethyl sulfoxide/methanol (1:1, 4 mL) and purified by reverse phase HPLC (Gilson system), eluting with 10-85% acetonitrile in 0.1% trifluoroacetic acid in water, to give the title compound. MS (ESI) m/e 1015.2 (M+H)⁺.

### 1.9.2. N-[6-(2,5-dioxo-2,5-dihydro-1H-pyrrol-1-yl)hexanoyl]-L-valyl-N-{4-[({[4-(beta-D-allopyranosyloxy)benzyl][2-({3-[(4-{6-[8-(1,3-benzothiazol-2-ylcarbamoyl)-3,4-dihydroisoquinolin-2(1H)-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl]carbamoyl}oxy)methyl]phenyl}-N⁵-carbamoyl-L-ornithinamide

The title compound was prepared by substituting Example 2.9.1 for Example 2.5.3 in Example 2.5.4. ¹H NMR (500 MHz, dimethyl sulfoxide-d₆) δ ppm 12.86 (s, 1H), 10.00 (s, 1H), 7.96-8.11 (m, 2H), 7.79 (d, 2H), 7.53-7.65 (m, 3H), 7.08-7.52 (m, 10H), 6.91-7.00 (m, 5H), 5.09 (d, 1H), 4.99 (d, 4H), 4.35-4.48 (m, 3H), 4.13-4.23 (m, 2H), 3.82-3.96 (m, 8H), 3.32-3.50 (m, 10H), 3.12-3.25 (m, 3H), 2.90-3.06 (m, 5H), 1.89-2.19 (m, 6H), 0.88-1.75 (m, 22H), 0.76-0.88 (m, 11H). MS (ESI) m/e 1612.5 (M+H)⁺.

### 1.10. Synthesis of N-[6-(2,5-dioxo-2,5-dihydro-1H-pyrrol-1-yl)hexanoyl]-L-valyl-N-{4-[({[2-({3-[(4-{6-[8-(1,3-benzothiazol-2-ylcarbamoyl)-3,4-dihydroisoquinolin-2(1H)-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](2-phosphonoethyl)carbamoyl}oxy)methyl]phenyl}-N⁵-carbamoyl-L-ornithinamide (Synthon EQ)

The title compound was prepared as described in Example 2.1, replacing Example 1.2.9 with Example 1.12.2. ¹H NMR (500 MHz, dimethyl sulfoxide-d₆) δ ppm 9.99 (s, 1H), 8.01-8.09 (m, 2H), 7.76-7.81 (m, 2H), 7.56-7.64 (m, 3H), 7.41-7.53 (m, 3H), 7.36 (q, 2H), 7.25-7.30 (m, 3H), 6.99 (s, 2H), 6.94 (d, 1H), 5.98 (s, 1H), 4.89-5.07 (m, 4H), 4.38 (s, 1H), 4.19 (t, 1H), 3.88 (t, 2H), 3.80 (d, 2H), 2.89-3.08 (m, 5H), 2.04-2.24 (m, 5H), 1.89-2.02 (m, 1H), 1.76-1.87 (m, 2H), 0.89-1.72 (m, 23H), 0.78-0.88 (m, 12H). MS (ESI) m/e 1452.2 (M+H)⁺.

### 1.11. Synthesis of N-[6-(2,5-dioxo-2,5-dihydro-1H-pyrrol-1-yl)hexanoyl]-L-valyl-N-{4-[({[2-({3-[(4-{6-[8-(1,3-benzothiazol-2-ylcarbamoyl)-3,4-dihydroisoquinolin-2(1H)-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](2-phosphonoethyl)carbamoyl}oxy)methyl]phenyl}-L-alaninamide (Synthon EU)

The title compound was prepared as described in Example 2.1, replacing Example 1.2.9 and 4-((S)-2-((S)-2-(6-(2,5-dioxo-2,5-dihydro-1H-pyrrol-1-yl)hexanamido)-3-methylbutanamido)-5-ureidopentanamido)benzyl (4-nitrophenyl) carbonate with Example 1.12.2 and 4-((S)-2-((S)-2-(6-(2,5-dioxo-2,5-dihydro-1H-pyrrol-1-yl)hexanamido)-3-methylbutanamido)propanamido)benzyl (4-nitrophenyl) carbonate, respectively. ¹H NMR (400 MHz, dimethyl sulfoxide-d₆) δ ppm 9.93 (s, 1H), 8.12 (d, 1H), 8.03 (d, 1H), 7.72-7.83 (m, 2H), 7.54-7.65 (m, 3H), 7.41-7.54 (m, 3H), 7.31-7.40 (m, 2H), 7.24-7.30 (m, 3H), 6.99 (s, 2H), 6.94 (d, 1H), 4.87-5.11 (m, 3H), 4.11-4.45 (m, 1H), 3.88 (t, 2H), 3.79 (d, 2H), 2.97-3.05 (m, 2H), 2.63-2.70 (m, 1H), 2.29-2.37 (m, 1H), 2.03-2.20 (m, 5H), 1.73-2.00 (m, 5H), 1.39-1.55 (m, 4H), 0.88-1.38 (m, 19H), 0.72-0.89 (m, 12H). MS (ESI) m/e 1364.5 (M−H)⁻.

### 1.12. Synthesis of N-[6-(2,5-dioxo-2,5-dihydro-1H-pyrrol-1-yl)hexanoyl]-L-valyl-N-{(4-[({[2-({3-[(4-{6-[8-(1,3-benzothiazol-2-ylcarbamoyl)-3,4-dihydroisoquinolin-2(1H)-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](3-phosphonopropyl)carbamoyl}oxy)methyl]phenyl}-N⁵-carbamoyl-L-ornithinamide (Synthon EV)

The title compound was prepared as described in Example 2.1, replacing Example 1.2.9 with Example 1.14.4. ¹H NMR (400 MHz, dimethyl sulfoxide-d₆) δ ppm 9.98 (s, 1H), 8.04 (t, 2H), 7.78 (t, 2H), 7.61 (t, 3H), 7.39-7.54 (m, 3H), 7.32-7.39 (m, 2H), 7.25-7.30 (m, 3H), 6.99 (s, 2H), 6.95 (d, 1H), 6.01 (s, 1H), 4.97 (d, 4H), 4.29-4.47 (m, 2H), 4.14-4.23 (m, 2H), 3.85-3.93 (m, 2H), 3.32-3.42 (m, 2H), 3.24 (s, 2H), 2.88-3.09 (m, 3H), 1.87-2.23 (m, 6H), 0.91-1.74 (m, 27H), 0.72-0.89 (m, 12H). MS (ESI) m/e 1466.3 (M+H)⁺.

### 1.13. Synthesis of N-[6-(2,5-dioxo-2,5-dihydro-1H-pyrrol-1-yl)hexanoyl]-L-valyl-N-({4-[({[(2R)-1-{[2-({3-[(4-{6-[8-(1,3-benzothiazol-2-ylcarbamoyl)-3,4-dihydroisoquinolin-2(1H)-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl]amino}-1-oxo-3-sulfopropan-2-yl]carbamoyl}oxy)methyl]phenyl}-L-alaninamide (Synthon EW)

To a solution of Example 1.15 (0.020 g) and 4-((S)-2-((S)-2-(6-(2,5-dioxo-2,5-dihydro-1H-pyrrol-1-yl)hexanamido)-3-methylbutanamido)propanamido)benzyl (4-nitrophenyl) carbonate (0.017 g) in N,N-dimethylformamide (0.5 mL) was added N,N-diisopropylethylamine (0.017 mL). The reaction was stirred overnight and was diluted with N,N-dimethylformamide (1 mL), water (0.5 mL). The mixture was purified by reverse phase HPLC using a Gilson system, eluting with 10-70% acetonitrile in water containing 0.1% v/v trifluoroacetic acid. The desired fractions were combined and freeze-dried to provide the title compound. ¹H NMR (400 MHz, dimethyl sulfoxide-d₆) δ ppm 12.85 (s, 1H), 9.93 (s, 1H), 8.12 (d, 1H), 8.04 (d, 1H), 7.86-7.76 (m, 3H), 7.63-7.41 (m, 7H), 7.39-7.32 (m, 2H), 7.30 (s, 1H), 7.30-7.21 (m, 2H), 6.99 (s, 2H), 6.97 (d, 1H), 4.96 (s, 2H), 4.93 (s, 2H), 4.49-4.33 (m, 2H), 4.18 (dd, 2H), 4.15-4.08 (m, 2H), 3.90-3.86 (m, 2H), 3.36 (t, 2H), 3.34-3.27 (m, 1H), 3.18-3.04 (m, 2H), 3.04-2.96 (m, 2H), 2.89-2.61 (m, 2H), 2.27-2.05 (m, 5H), 2.03-1.87 (m, 1H), 1.59-1.42 (m, 4H), 1.42-0.91 (m, 18H), 0.91-0.76 (m, 11H). MS (−ESI) m/e 1407.5 (M−H)⁻.

### 1.14. Synthesis of N-[6-(2,5-dioxo-2,5-dihydro-1H-pyrrol-1-yl)hexanoyl]-L-valyl-N-[4-({[{2-[2-({3-[(4-{6-[8-(1,3-benzothiazol-2-ylcarbamoyl)-3,4-dihydroisoquinolin-2(1H)-yl]-2-carboxypyridin-3-yl}-5-methyl-1H-pyrazol-1-yl)methyl]-5,7-dimethyltricyclo[3.3.1.1^{3,7}]dec-1-yl}oxy)ethoxy]ethyl}(3-phosphonopropyl)carbamoyl]oxy}methyl)phenyl]-N⁵-carbamoyl-L-ornithinamide (Synthon EX)

A mixture of Example 1.16.2 (59 mg), 4-((S)-2-((S)-2-(6-(2,5-dioxo-2,5-dihydro-1H-pyrrol-1-yl)hexanamido)-3-methylbutanamido)-5-ureidopentanamido)benzyl (4-nitrophenyl) carbonate (48 mg), and N,N-diisopropylethylamine (0.056 mL) in 2 mL N,N-dimethylformamide was stirred for 24 hours. The mixture was purified via reverse phase chromatography on a Biotage Isolera One system using a 40 g C18 column, eluting with 10-90% acetonitrile in 0.1% trifluoroacetic acid/water. The desired fractions were concentrated and the product was lyophilized from water and 1,4-dioxane to give the title compound as a trifluoroacetic acid salt. ¹H NMR (400 MHz, dimethyl sulfoxide-d₆) δ ppm 9.97 (bs, 1H), 8.04 (m, 2H), 7.79 (d, 2H), 7.59 (m, 3H), 7.46 (m, 3H), 7.36 (m, 2H), 7.27 (m, 2H), 6.99 (s, 2H), 6.94 (d, 1H), 4.97 (m, 4H), 4.40 (m, 2H), 4.17 (dd, 2H), 3.50-4.10 (m, 6H), 3.45 (m, 2H), 3.40 (m, 2H), 3.26 (m, 2H), 3.01 (m, 2H), 2.95 (s, 2H), 2.79 (s, 2H), 2.15 (m, 2H), 2.09 (s, 2H), 1.68 (m, 2H), 1.60 (m, 1-2H), 1.35-1.50 (m, 6H), 1.25 (m, 4H), 1.17 (m, 2H), 1.10 (m, 2H), 0.97 (m, 1-2H), 0.84 (m, 12H). MS (ESI) m/e 1510.4 (M+H)⁺.

### 1.15. Synthesis of N-[6-(2,5-dioxo-2,5-dihydro-1H-pyrrol-1-yl)hexanoyl]-L-valyl-N-[4-({[{2-[2-({3-[(4-{6-[8-(1,3-benzothiazol-2-ylcarbamoyl)-3,4-dihydroisoquinolin-2(1H)-yl]-2-carboxypyridin-3-yl}-5-methyl-1H-pyrazol-1-yl)methyl]-5,7-dimethyltricyclo[3.3.1.1^{3,7}]dec-1-yl}oxy)ethoxy]ethyl}(3-phosphonopropyl)carbamoyl]oxy}methyl)phenyl]-L-alaninamide (Synthon EY)

A mixture of Example 1.16.2 (59 mg), 4-((S)-2-((S)-2-(6-(2,5-dioxo-2,5-dihydro-1H-pyrrol-1-yl)hexanamido)-3-methylbutanamido)propanamido)benzyl (4-nitrophenyl) carbonate (42 mg), and N,N-diisopropylethylamine (0.042 mg) in 2 mL N,N-dimethylformamide was stirred for 24 hours. The mixture was purified via reverse phase chromatography on a Biotage Isolera One system using a 40 g C18 column, eluting with 10-90% acetonitrile in 0.1% trifluoroacetic acid/water. Fractions were concentrated and the product was lyophilized from water and 1,4-dioxane to give the title compound as a trifluoroacetic acid salt. MS (ESI) m/e 1422.6 (M−H)⁺.

### 1.16. Synthesis of N-[6-(2,5-dioxo-2,5-dihydro-1H-pyrrol-1-yl)hexanoyl]-L-valyl-N-{4-[({[2-({3-[(4-{6-[8-(1,3-benzothiazol-2-ylcarbamoyl)-3,4-dihydroisoquinolin-2(1H)-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](3-phosphonopropyl)carbamoyl}oxy)methyl]phenyl}-L-alaninamide (Synthon EZ)

A mixture of Example 1.14.4 (50 mg), 4-((S)-2-((S)-2-(6-(2,5-dioxo-2,5-dihydro-1H-pyrrol-1-yl)hexanamido)-3-methylbutanamido)propanamido)benzyl (4-nitrophenyl) carbonate (38 mg), and N,N-diisopropylethylamine (0.050 mL) in 2 mL N,N-dimethylformamide was stirred for 24 hours. The mixture was purified via reverse phase chromatography on a Biotage Isolera One system using a 40 g C18 column, eluting with 10-90% acetonitrile in 0.1% trifluoroacetic acid/water. The desired fractions were concentrated and the product was lyophilized from water and 1,4-dioxane to give the title compound as a trifluoroacetic acid salt. ¹H NMR (400 MHz, dimethyl sulfoxide-d₆) S ppm 9.94 (bs, 1H), 8.12 (d, 1H), 8.04 (d, 1H), 7.80 (d, 2H), 7.61 (m, 3H), 7.47 (m, 3H), 7.36 (m, 2H), 7.29 (m, 2H), 6.99 (s, 2H), 6.95 (d, 1H), 4.97 (m, 4H), 4.40 (m, 2H), 4.16 (dd, 2H), 3.50-4.10 (m, 6H), 3.68 (m, 2H), 3.55 (m, 2H), 3.25 (m, 4H), 3.02 (m, 2H), 2.94 (s, 2H), 2.79 (s, 2H), 2.15 (m, 1H), 2.08 (s, 2H), 1.65 (m, 2H), 1.40-1.50 (m, 6H), 1.20-1.30 (m, 6H), 1.08-1.19 (m, 4H), 0.97 (m, 1-2H), 0.76-0.89 (m, 12H). MS (ESI) m/e 1380.3 (M+H)⁺.

### 1.17. Synthesis of 6-[8-(1,3-benzothiazol-2-ylcarbamoyl)-3,4-dihydroisoquinolin-2(1H)-yl]-3-(1-{[3-(2-{[(2S)-3-carboxy-2-({[(4-{[(2S)-2-{[(2S)-2-{[6-(2,5-dioxo-2,5-dihydro-1H-pyrrol-1-yl)hexanoyl]amino)-3-methylbutanoyl]amino}propanoyl]amino}benzyl)oxy]carbonyl}amino)propanoyl](methyl)amino}ethoxy)-5,7-dimethyltricyclo[3.3.1.1^{3,7}]dec-1-yl]methyl}-5-methyl-1H-pyrazol-4-yl}pyridine-2-carboxylic Acid (Synthon FD)

To a solution of Example 1.17 (0.040 g) and 4-((S)-2-((S)-2-(6-(2,5-dioxo-2,5-dihydro-1H-pyrrol-1-yl)hexanamido)-3-methylbutanamido)propanamido)benzyl (4-nitrophenyl) carbonate (0.034 g) in N,N-dimethylformamide (1 mL) was added N,N-diisopropylethylamine (0.035 mL). The reaction was stirred overnight and diluted with N,N-dimethylformamide (1 mL) and water (0.5 mL). The mixture was purified by reverse phase HPLC using a Gilson system, eluting with 10-70% acetonitrile in water containing 0.1% v/v trifluoroacetic acid. The desired fractions were combined and freeze-dried to provide the title compound. ¹H NMR (400 MHz, dimethyl sulfoxide-d₆) δ ppm 12.84 (s, 1H), 9.92 (s, 1H), 8.13 (d, 1H), 8.03 (d, 1H), 7.79 (d, 2H), 7.62 (d, 1H), 7.57 (d, 2H), 7.54-7.41 (m, 3H), 7.40-7.32 (m, 2H), 7.31-7.23 (m, 4H), 6.99 (s, 2H), 6.95 (dd, 1H), 5.01-4.89 (m, 4H), 4.78 (dq, 1H), 4.45-4.30 (m, 1H), 4.23-4.11 (m, 1H), 3.88 (t, 2H), 3.80 (s, 2H), 3.42-3.26 (m, 6H), 3.06 (s, 1H), 3.01 (t, 2H), 2.80 (s, 2H), 2.76-2.62 (m, 1H), 2.46-2.36 (m, 1H), 2.25-2.05 (m, 5H), 2.05-1.92 (m, 1H), 1.58-1.42 (m, 4H), 1.42-0.91 (m, 20H), 0.91-0.78 (m, 9H). MS (ESI) m/e 1387.4 (M+H)⁺.

### 1.18. Synthesis of N-[6-(2,5-dioxo-2,5-dihydro-1H-pyrrol-1-yl)hexanoyl]-L-valyl-N-{4-[({[2-({3-[(4-{6-[8-(1,3-benzothiazol-2-ylcarbamoyl)-3,4-dihydroisoquinolin-2(1H)-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl][4-(beta-D-glucopyranuronosyloxy)benzyl]carbamoyl}oxy)methyl]phenyl}-N⁵-carbamoyl-L-ornithinamide (Synthon FS)

The title compound was prepared by substituting Example 1.19.2 for Example 2.5.3 in Example 2.5.4. ¹H NMR (500 MHz, dimethyl sulfoxide-d₆) δ ppm 12.86 (s, 1H), 10.00 (s, 1H), 7.97-8.14 (m, 2H), 7.79 (d, 2H), 7.07-7.65 (m, 13H), 6.87-7.01 (m, 4H), 5.92-6.08 (m, 1H), 4.87-5.07 (m, 4H), 4.33-4.48 (m, 3H), 4.13-4.26 (m, 1H), 3.74-3.94 (m, 6H), 3.14-3.34 (m, 8H), 2.84-3.05 (m, 6H), 1.87-2.25 (m, 6H), 0.89-1.73 (m, 21H), 0.76-0.87 (m, 12H). MS (ESI) m/e 1626.4 (M+H)⁺.

### 1.19. Synthesis of N-[6-(2,5-dioxo-2,5-dihydro-1H-pyrrol-1-yl)hexanoyl]-L-valyl-N-{4-[({[2-({3-[(4-{6-[1-(1,3-benzothiazol-2-ylcarbamoyl)-1,2,3,4-tetrahydroquinolin-7-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](2-phosphonoethyl)carbamoyl}oxy)methyl]phenyl}-N⁵-carbamoyl-L-ornithinamide (Synthon FI)

The title compound was prepared as described in Example 2.1, replacing Example 1.2.9 with Example 1.20.11. ¹H NMR (500 MHz, dimethyl sulfoxide-d₆) δ ppm 10.00 (s, 1H), 8.40 (s, 1H), 8.07 (d, 1H), 8.00 (d, 1H), 7.84-7.90 (m, 1H), 7.79 (dd, 3H), 7.55-7.66 (m, 2H), 7.46 (s, 2H), 7.37 (t, 1H), 7.29 (t, 3H), 7.18-7.25 (m, 1H), 6.99 (s, 2H), 5.99 (s, 1H), 5.00 (d, 1H), 4.38 (s, 1H), 4.13-4.24 (m, 1H), 3.96 (s, 2H), 3.87 (d, 2H), 2.88-3.08 (m, 4H), 2.84 (q, 2H), 2.04-2.26 (m, 5H), 1.89-2.01 (m, 3H), 1.75-1.88 (m, 2H), 1.63-1.74 (m, 1H), 0.91-1.63 (m, 21H), 0.76-0.89 (m, 12H). MS (ESI) m/e 1450.5 (M−H)⁻.

### 1.20. Synthesis of N-[6-(2,5-dioxo-2,5-dihydro-1H-pyrrol-1-yl)hexanoyl]-L-valyl-N⁵-carbamoyl-N-{4-[({[2-({3-[(4-{2-carboxy-6-[8-([1,3]thiazolo[5,4-b]pyridin-2-ylcarbamoyl)-3,4-dihydroisoquinolin-2(1H)-yl]pyridin-3-yl}-5-methyl-1H-pyrazol-1-yl)methyl]-5,7-dimethyltricyclo[3.3.1.1^{3,7}]dec-1-yl}oxy)ethyl](2-sulfoethyl)carbamoyl}oxy)methyl]phenyl}-L-ornithinamide (Synthon FV)

The title compound was prepared by substituting Example 1.22.5 for Example 1.2.9 in Example 2.1. ¹H NMR (500 MHz, dimethyl sulfoxide-d₆) δ ppm 13.00 (v br s, 1H), 10.00 (s, 1H), 8.52 (dd, 1H), 8.16 (dd, 1H), 8.06 (d, 1H), 7.78 (d, 1H), 7.62 (d, 1H), 7.59 (br m, 2H), 7.53 (m, 2H), 7.45 (d, 1H), 7.37 (t, 1H), 7.30 (s, 1H) 7.27 (d, 2H), 6.99 (s, 2H), 6.97 (d, 1H), 4.98 (m, 4H), 4.39 (m, 1H), 4.19 (br m, 1H), 3.88 (t, 2H), 3.80 (br d, 2H), 3.44, 3.36 (br m, m, total 6H), 3.24 (m, 2H), 2.94-3.01 (m, 4H), 2.63 (br m, 2H), 2.14 (m, 2H), 2.10 (s, 3H), 1.97 (br m, 1H), 1.68 (br m, 1H), 1.58 (br m, 1H), 1.34-1.47 (m, 8H), 1.08-1.23 (m 10H), 0.95 (br m, 2H), 0.85-0.80 (m, 12H). MS (ESI) m/e 1451.4 (M−H)⁻.

### 1.21. Synthesis of N-[6-(2,5-dioxo-2,5-dihydro-1H-pyrrol-1-yl)hexanoyl]-L-valyl-N-({4-[({[(2R)-{([2-({3-[(4-{6-[1-(1,3-benzothiazol-2-ylcarbamoyl)-1,2,3,4-tetrahydroquinolin-7-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](methyl)amino}-1-oxo-3-sulfopropan-2-yl]carbamoyl}oxy)methyl]phenyl}-N⁵-carbamoyl-L-ornithinamide (Synthon GC)

The title compound was prepared as described in Example 2.1, replacing Example 1.2.9 with Example 1.21.7. ¹H NMR (500 MHz, dimethyl sulfoxide-d₆) δ ppm 9.98 (s, 1H), 8.40 (s, 1H), 8.07 (d, 1H), 8.01 (dd, 1H), 7.89 (t, 1H), 7.74-7.84 (m, 3H), 7.58 (d, 2H), 7.47 (s, 2H), 7.37 (t, 1H), 7.19-7.33 (m, 5H), 7.00 (s, 2H), 4.91 (q, 2H), 4.64-4.76 (m, 2H), 4.33-4.43 (m, 2H), 4.15-4.24 (m, 2H), 3.92-4.03 (m, 2H), 3.88 (s, 2H), 3.32-3.50 (m, 6H), 3.10-3.22 (m, 2H), 2.89-3.07 (m, 2H), 2.70-2.89 (m, 4H), 2.60-2.70 (m, 1H), 2.05-2.28 (m, 5H), 1.90-2.03 (m, 3H), 1.64-1.77 (m, 1H), 1.53-1.65 (m, 1H), 0.92-1.53 (m, 21H), 0.77-0.92 (m, 12H). MS (ESI) m/e 1507.3 (M−H)⁻.

### 1.22. Synthesis of N-[6-(2,5-dioxo-2,5-dihydro-1H-pyrrol-1-yl)hexanoyl]-L-valyl-N-({4-[({[(2R)-1{([2-({3-[(4-{6-[1-(1,3-benzothiazol-2-ylcarbamoyl)-1,2,3,4-tetrahydroquinolin-7-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](methyl)amino}-1-oxo-3-sulfopropan-2-yl]carbamoyl}oxy)methyl]phenyl}-L-alaninamide (Synthon GB)

The title compound was prepared as described in Example 2.1, replacing Example 1.2.9 and 4-((S)-2-((S)-2-(6-(2,5-dioxo-2,5-dihydro-1H-pyrrol-1-yl)hexanamido)-3-methylbutanamido)-5-ureidopentanamido)benzyl (4-nitrophenyl) carbonate with Example 1.21.7 and 4-((S)-2-((S)-2-(6-(2,5-dioxo-2,5-dihydro-1H-pyrrol-1-yl)hexanamido)-3-methylbutanamido)propanamido)benzyl (4-nitrophenyl) carbonate, respectively. ¹H NMR (500 MHz, dimethyl sulfoxide-d₆) δ ppm 9.93 (s, 1H), 8.39 (s, 1H), 8.13 (d, 1H), 8.01 (dd, 1H), 7.88 (t, 1H), 7.74-7.84 (m, 3H), 7.57 (d, 2H), 7.46 (s, 2H), 7.37 (t, 1H), 7.17-7.33 (m, 5H), 6.99 (s, 2H), 4.91 (d, 2H), 4.65-4.76 (m, 1H), 4.30-4.51 (m, 1H), 4.13-4.21 (m, 1H), 3.92-4.00 (m, 2H), 3.88 (s, 2H), 3.29-3.46 (m, 4H), 2.93-3.21 (m, 3H), 2.68-2.88 (m, 4H), 2.58-2.68 (m, 1H), 2.04-2.26 (m, 5H), 1.89-2.02 (m, 3H), 1.37-1.54 (m, 6H), 0.92-1.34 (m, 15H), 0.75-0.91 (m, 12H). MS (ESI) m/e (M+H)⁺.

### 1.23. Synthesis of N-[6-(2,5-dioxo-2,5-dihydro-1H-pyrrol-1-yl)hexanoyl]-L-valyl-N⁵-carbamoyl-N-{4-[({[2-({3-[(4-{2-carboxy-6-[8-([1,3]thiazolo[4,5-b]pyridin-2-ylcarbamoyl)-3,4-dihydroisoquinolin-2(1H)-yl]pyridin-3-yl}-5-methyl-1H-pyrazol-1-yl)methyl]-5,7-dimethyltricyclo[3.3.1.1^{3,7}]dec-1-yl}oxy)ethyl](2-sulfoethyl)carbamoyl}oxy)methyl]phenyl}-L-ornithinamide (Synthon FW)

The title compound was prepared by substituting Example 1.23.4 for Example 1.2.9 in Example 2.1. ¹H NMR (500 MHz, dimethyl sulfoxide-d₆) δ ppm 13.38 (v br s, 1H), 10.00 (s, 1H), 8.66 (m, 2H), 8.06 (d, 1H), 7.78 (d, 1H), 7.65 (d, 1H), 7.59 (br m, 2H), 7.53 (m, 1H), 7.47 (m 2H), 7.37 (t, 1H), 7.30 (s, 1H) 7.27 (d, 2H), 6.99 (s, 2H), 6.97 (d, 1H), 4.98 (m, 4H), 4.39 (m, 1H), 4.19 (br m, 1H), 3.88 (t, 2H), 3.80 (br d, 2H), 3.40 (br m, 6H), 3.24 (m, 2H), 2.98 (m, 4H), 2.63 (m, 2H), 2.16 (m, 2H), 2.10 (s, 3H), 1.97 (br m, 1H), 1.68 (br m, 1H), 1.58 (br m, 1H), 1.34-1.47 (m, 8H), 1.08-1.23 (m, 10H), 0.95 (br m, 2H), 0.85-0.80 (m, 12H). MS (ESI) m/e 1451.5 (M−H)⁻.

### 1.24. Synthesis of N-[6-(2,5-dioxo-2,5-dihydro-1H-pyrrol-1-yl)hexanoyl]-L-valyl-N-{4-[({[2-({3-[(4-{6-[1-(1,3-benzothiazol-2-ylcarbamoyl)-1,2,3,4-tetrahydroquinolin-7-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](2-sulfoethyl)carbamoyl}oxy)methyl]phenyl}-N⁵-carbamoyl-L-ornithinamide (Synthon GD)

The title compound was prepared as described in Example 2.1, replacing Example 1.2.9 with Example 1.24.2. ¹H NMR (500 MHz, dimethyl sulfoxide-d₆) δ ppm 10.00 (s, 1H), 8.38 (s, 1H), 8.07 (d, 1H), 8.00 (d, 1H), 7.85-7.92 (m, 1H), 7.73-7.85 (m, 3H), 7.55-7.65 (m, 2H), 7.46 (s, 2H), 7.37 (t, 1H), 7.28 (t, 3H), 7.22 (t, 1H), 6.99 (s, 2H), 6.00 (s, 1H), 4.99 (d, 1H), 4.28-4.50 (m, 1H), 4.19 (s, 1H), 3.77-4.03 (m, 4H), 3.31-3.41 (m, 2H), 3.20-3.29 (m, 2H), 2.87-3.08 (m, 3H), 2.83 (t, 2H), 2.63 (d, 2H), 2.05-2.25 (m, 5H), 1.88-2.01 (m, 3H), 1.69 (t, 1H), 1.53-1.63 (m, 1H), 1.31-1.53 (m, 8H), 1.04-1.29 (m, 11H), 0.89-1.02 (m, 2H), 0.77-0.88 (m, 12H). MS (ESI) m/e 1450.4 (M−H)⁻.

### 1.25. Synthesis of N-[6-(2,5-dioxo-2,5-dihydro-1H-pyrrol-1-yl)hexanoyl]-L-valyl-N-{4-[({[2-({3-[(4-{6-[8-(1,3-benzothiazol-2-ylcarbamoyl)-3,4-dihydroisoquinolin-2(1H)-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](2-carboxyethyl)carbamoyl}oxy)methyl]phenyl}-N⁵-carbamoyl-L-ornithinamide (Synthon GK)

The title compound was prepared as described in Example 2.1, replacing Example 1.2.9 with Example 1.25.2. ¹H NMR (400 MHz, dimethyl sulfoxide-d₆) δ ppm 12.85 (s, 1H), 9.98 (s, 1H), 8.04 (t, 2H), 7.75-7.82 (m, 2H), 7.60 (t, 3H), 7.41-7.53 (m, 3H), 7.32-7.39 (m, 2H), 7.24-7.29 (m, 3H), 6.99 (s, 2H), 6.94 (d, 3H), 5.97 (s, 1H), 4.88-5.04 (m, 4H), 4.38 (d, 1H), 4.12-4.24 (m, 1H), 3.88 (t, 2H), 3.75-3.84 (m, 2H), 3.32-3.40 (m, 2H), 3.28 (d, 2H), 2.90-3.05 (m, 4H), 2.42-2.49 (m, 2H), 2.05-2.22 (m, 5H), 1.87-2.01 (m, 1H), 0.90-1.76 (m, 22H), 0.74-0.88 (m, 12H). MS (ESI) m/e 1414.5 (M−H)⁻.

### 1.26. Synthesis of N-[6-(2,5-dioxo-2,5-dihydro-1H-pyrrol-1-yl)hexanoyl]-L-valyl-N-(4-[({[2-({3-[(4-{6-[8-(1,3-benzothiazol-2-ylcarbamoyl)-3,4-dihydroisoquinolin-2(1H)-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](2-carboxyethyl)carbamoyl}oxy)methyl]phenyl}-L-alaninamide (Synthon GJ)

The title compound was prepared as described in Example 2.1, replacing Example 1.2.9 and 4-((S)-2-((S)-2-(6-(2,5-dioxo-2,5-dihydro-1H-pyrrol-1-yl)hexanamido)-3-methylbutanamido)-5-ureidopentanamido)benzyl (4-nitrophenyl) carbonate with Example 1.25.2 and 4-((S)-2-((S)-2-(6-(2,5-dioxo-2,5-dihydro-1H-pyrrol-1-yl)hexanamido)-3-methylbutanamido)propanamido)benzyl (4-nitrophenyl) carbonate, respectively. ¹H NMR (400 MHz, dimethyl sulfoxide-d₆) δ ppm 12.78 (s, 1H), 9.93 (s, 1H), 8.12 (d, 1H), 8.03 (d, 1H), 7.75-7.83 (m, 2H), 7.54-7.65 (m, 3H), 7.41-7.52 (m, 3H), 7.32-7.40 (m, 2H), 7.24-7.29 (m, 3H), 6.98 (s, 2H), 6.94 (d, 1H), 4.90-5.04 (m, 4H), 4.32-4.45 (m, 2H), 4.12-4.21 (m, 2H), 3.88 (t, 2H), 3.79 (d, 2H), 3.31-3.46 (m, 4H), 3.23-3.31 (m, 2H), 3.01 (t, 2H), 2.46 (t, 2H), 2.04-2.22 (m, 5H), 1.87-2.02 (m, 1H), 1.40-1.60 (m, 4H), 0.91-1.37 (m, 17H), 0.76-0.88 (m, 12H). MS (ESI) m/e 1328.4 (M−H)⁻.

### 1.27. Synthesis of 6-[8-(1,3-benzothiazol-2-ylcarbamoyl)-3,4-dihydroisoquinolin-2(1H)-yl]-3-(1-{[3-(2-{[(2R)-3-carboxy-2-({[(4-{[(2S)-2-{[(2S)-2-{[6-(2,5-dioxo-2,5-dihydro-1H-pyrrol-1-yl)hexanoyl]amino}-3-methylbutanoyl]amino}propanoyl]amino}benzyl)oxy]carbonyl}amino)propanoyl](methyl)amino}ethoxy)-5,7-dimethyltricyclo[3.3.1.1^{3,7}]dec-1-yl]methyl}-5-methyl-1H-pyrazol-4-yl)pyridine-2-carboxylic Acid (Synthon GW)

A solution of Example 1.27 (0.043 g) in N,N-dimethylformamide (0.5 mL) was added 4-((S)-2-((S)-2-(6-(2,5-dioxo-2,5-dihydro-1H-pyrrol-1-yl)hexanamido)-3-methylbutanamido)propanamido)benzyl (4-nitrophenyl) carbonate (0.042 g) followed by N,N-diisopropylethylamine (0.038 mL), and the reaction was stirred at room temperature. After stirring for 16 hours, the reaction was diluted with water (0.5 mL) and N,N-dimethylformamide (1 mL). The mixture was purified by reverse phase HPLC using a Gilson system, eluting with 10-70% acetonitrile in water containing 0.1% v/v trifluoroacetic acid. The desired fractions were combined and freeze-dried to provide the title compound. ¹H NMR (400 MHz, dimethyl sulfoxide-d₆) δ ppm 13.05 (s, 1H), 10.15 (s, 1H), 8.36 (d, 1H), 8.26 (d, 1H), 8.02 (d, 2H), 7.95-7.77 (m, 4H), 7.77-7.63 (m, 3H), 7.63-7.54 (m, 2H), 7.54-7.46 (m, 3H), 7.22 (s, 2H), 7.18 (dd, 1H), 5.17 (d, 4H), 5.01 (dq, 1H), 4.61 (p, 1H), 4.39 (t, 1H), 4.11 (t, 2H), 4.03 (s, 2H), 3.64-3.49 (m, 2H), 3.29 (s, 1H), 3.24 (t, 2H), 3.03 (s, 2H), 2.92 (dt, 1H), 2.73-2.61 (m, 4H), 2.35 (d, 4H), 2.18 (dt, 1H), 1.71 (h, 4H), 1.65-1.13 (m, 18H), 1.13-1.01 (m, 13H). MS (ESI) m/e 1387.3 (M+H)⁺.

### 1.28. Synthesis of N-[6-(2,5-dioxo-2,5-dihydro-1H-pyrrol-1-yl)hexanoyl]-L-valyl-N-{4-[({[2-({3-[(4-{6-[8-(1,3-benzothiazol-2-ylcarbamoyl)-3,4-dihydroisoquinolin-2(1H)-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl][1-(carboxymethyl)piperidin-4-yl]carbamoyl}oxy)methyl]phenyl}-N⁵-carbamoyl-L-ornithinamide (Synthon HF)

A solution of Example 1.28 (0.0449 g), 4-((S)-2-((S)-2-(6-(2,5-dioxo-2,5-dihydro-1H-pyrrol-1-yl)hexanamido)-3-methylbutanamido)-5-ureidopentanamido)benzyl (4-nitrophenyl) carbonate (0.049 g) and N,N-diisopropylethylamine (0.044 mL) were stirred together in N,N-dimethylformamide (0.5 mL) at room temperature. The reaction mixture was stirred overnight and diluted with N,N-dimethylformamide (1 mL) and water (0.5 mL). The mixture was purified by reverse phase HPLC using a Gilson system, eluting with 10-90% acetonitrile in water containing 0.1% v/v trifluoroacetic acid. The desired fractions were combined and freeze-dried to provide the title compound. ¹H NMR (400 MHz, dimethyl sulfoxide-d₆) δ ppm 12.85 (s, 1H), 9.99 (s, 1H), 8.04 (t, 2H), 7.78 (t, 2H), 7.65-7.58 (m, 3H), 7.54-7.41 (m, 3H), 7.38 (d, 1H), 7.34 (d, 1H), 7.32-7.24 (m, 3H), 6.99 (s, 2H), 6.95 (d, 1H), 5.97 (s, 1H), 5.01 (s, 2H), 4.96 (s, 2H), 4.38 (q, 1H), 4.23-4.14 (m, 1H), 4.05 (s, 2H), 3.88 (t, 2H), 3.80 (s, 2H), 3.36 (t, 2H), 3.26-2.86 (m, 8H), 2.27-2.02 (m, 6H), 2.02-1.86 (m, 2H), 1.86-1.75 (m, 2H), 1.75-1.54 (m, 2H), 1.54-0.90 (m, 24H), 0.89-0.72 (m, 14H). MS (ESI) m/e 1485.2 (M+H)⁺.

### 1.29. Synthesis of (S)-6-((2-((3-((4-(6-(8-(benzo[d]thiazol-2-ylcarbamoyl)-3,4-dihydroisoquinolin-2(1H)-yl)-2-carboxypyridin-3-yl)-5-methyl-1H-pyrazol-1-yl)methyl)-5,7-dimethyladamantan-1-yl)oxy)ethyl)methyl)amino)-5-((((4-((S)-2-((S)-2-(6-(2,5-dioxo-2,5-dihydro-1H-pyrrol-1-yl)hexanamido)-3-methylbutanamido)-5-ureidopentanamido)benzyl)oxy)carbonyl)amino)-N,N,N-trimethyl-6-oxohexan-1-aminium Salt (Synthon HG)

A solution of Example 1.29 (8 mg), 4-((S)-2-((S)-2-(6-(2,5-dioxo-2,5-dihydro-1H-pyrrol-1-yl)hexanamido)-3-methylbutanamido)-5-ureidopentanamido)benzyl (4-nitrophenyl) carbonate (8.24 mg) and N,N-diisopropylethylamine (7.50 μl, 0.043 mmol) in N,N-dimethylformamide (0.250 mL) was stirred at room temperature. After 3 hours, the reaction was diluted with N,N-dimethylformamide (1.25 mL) and water (0.5 mL). The mixture was purified by reverse phase HPLC using a Gilson system, eluting with 10-90% acetonitrile in water containing 0.1% v/v trifluoroacetic acid. The desired fractions were combined and freeze-dried to provide the title compound. ¹H NMR (400 MHz, dimethyl sulfoxide-d₆) δ ppm 12.85 (s, 1H), 9.96 (s, 1H), 8.04 (t, 2H), 7.83-7.76 (m, 2H), 7.66-7.56 (m, 3H), 7.53-7.42 (m, 4H), 7.41-7.32 (m, 2H), 7.31-7.23 (m, 3H), 6.99 (s, 2H), 6.95 (d, 1H), 5.99 (s, 1H), 5.04-4.87 (m, 4H), 4.44-4.33 (m, 2H), 4.24-4.12 (m, 2H), 3.88 (t, 2H), 3.81 (s, 2H), 3.50-3.13 (m, 9H), 3.11-2.92 (m, 14H), 2.80 (s, 1H), 2.25-2.04 (m, 5H), 2.03-1.89 (m, 1H), 1.75-0.91 (m, 28H), 0.91-0.77 (m, 12H). MS (ESI) m/e 1528.5 (M+H)⁺.

### 1.30. Synthesis of N-[6-(2,5-dioxo-2,5-dihydro-1H-pyrrol-1-yl)hexanoyl]-L-valyl-N-{4-[({[2-({3-[(4-{6-[8-(1,3-benzothiazol-2-ylcarbamoyl)-3,4-dihydroisoquinolin-2(1H)-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](2-sulfoethyl)carbamoyl}oxy)methyl]phenyl}-L-alaninamide (Synthon HP)

The title compound was prepared as described in Example 2.1, replacing 4-((S)-2-((S)-2-(6-(2,5-dioxo-2,5-dihydro-1H-pyrrol-1-yl)hexanamido)-3-methylbutanamido)-5-ureidopentanamido)benzyl (4-nitrophenyl) carbonate with 4-((S)-2-((S)-2-(6-(2,5-dioxo-2,5-dihydro-1H-pyrrol-1-yl)hexanamido)-3-methylbutanamido)propanamido)benzyl (4-nitrophenyl) carbonate. ¹H NMR (400 MHz, dimethyl sulfoxide-d₆) δ ppm 12.83 (s, 1H), 9.94 (s, 1H), 8.12 (d, 1H), 8.04 (d, 1H), 7.79 (d, 2H), 7.40-7.63 (m, 6H), 7.32-7.39 (m, 2H), 7.24-7.30 (m, 3H), 6.99 (s, 2H), 6.95 (d, 1H), 4.90-5.03 (m, 4H), 4.31-4.47 (m, 1H), 4.09-4.24 (m, 1H), 3.84-3.93 (m, 2H), 3.81 (s, 2H), 3.30-3.39 (m, 2H), 3.20-3.28 (m, 2H), 3.01 (t, 2H), 2.57-2.65 (m, 2H), 2.05-2.22 (m, 5H), 1.87-2.02 (m, 2H), 1.41-1.58 (m, 4H), 1.22 (d, 18H), 0.74-0.89 (m, 12H). MS (ESI) m/e 1364.5 (M−H)⁻.

### 1.31. Synthesis of N-[6-(2,5-dioxo-2,5-dihydro-1H-pyrrol-1-yl)hexanoyl]-L-valyl-N-[4-({[(4-{[2-({3-[(4-{6-[8-(1,3-benzothiazol-2-ylcarbamoyl)-3,4-dihydroisoquinolin-2(1H)-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](2-sulfoethyl)amino}piperidin-1-yl)carbonyl]oxy}methyl)phenyl]-N⁵-carbamoyl-L-ornithinamide (Synthon HR)

A solution of Example 1.30.2 (0.038 g), 4-((S)-2-((S)-2-(6-(2,5-dioxo-2,5-dihydro-1H-pyrrol-1-yl)hexanamido)-3-methylbutanamido)-5-ureidopentanamido)benzyl (4-nitrophenyl) carbonate (0.035 g) and N,N-diisopropylethylamine (0.032 mL) in N,N-dimethylformamide (0.5 mL) was stirred at room temperature. After stirring for 3 hours, the reaction was diluted with N,N-dimethylformamide (1.25 mL) and water (0.5 mL). The mixture was purified by reverse phase HPLC using a Gilson system, eluting with 10-90% acetonitrile in water containing 0.1% v/v trifluoroacetic acid. The desired fractions were combined and freeze-dried to provide the title compound. ¹H NMR (400 MHz, dimethyl sulfoxide-d₆) δ ppm 9.98 (s, 1H), 9.02 (s, 1H), 8.10-8.00 (m, 2H), 7.79 (d, 2H), 7.64-7.56 (m, 3H), 7.53 (d, 1H), 7.47 (t, 1H), 7.43 (d, 1H), 7.39-7.32 (m, 2H), 7.29 (d, 3H), 6.99 (s, 2H), 6.95 (d, 1H), 6.00 (s, 1H), 4.99 (s, 2H), 4.96 (s, 2H), 4.48-4.32 (m, 2H), 4.27-4.15 (m, 2H), 4.11 (d, 2H), 3.88 (t, 2H), 3.82 (s, 2H), 3.40-3.33 (m, 4H), 3.24-3.11 (m, 2H), 3.11-2.72 (m, 8H), 2.26-2.04 (m, 4H), 2.04-1.80 (m, 3H), 1.80-0.92 (m, 26H), 0.92-0.77 (m, 12H). MS (ESI) m/e 1535.4 (M+H)⁺.

### 1.32. Synthesis of N-[6-(2,5-dioxo-2,5-dihydro-1H-pyrrol-1-yl)hexanoyl]-L-valyl-N-{4-[({[2-({3-[(4-{6-[8-(1,3-benzothiazol-2-ylcarbamoyl)-5-(3-phosphonopropoxy)-3,4-dihydroisoquinolin-2(1H)-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](methyl)carbamoyl}oxy)methyl]phenyl}-N⁵-carbamoyl-L-ornithinamide (Synthon HU)

The title compound was prepared by substituting Example 1.31.11 for Example 2.5.3 in Example 2.5.4. ¹H NMR (400 MHz, dimethyl sulfoxide-d₆) δ ppm 9.98 (s, 1H), 8.03 (dd, 2H), 7.70-7.84 (m, 3H), 7.59 (d, 2H), 7.48 (dd, 2H), 7.23-7.37 (m, 4H), 6.93-7.02 (m, 4H), 4.99 (d, 4H), 4.12-4.21 (m, 8H), 3.88-3.96 (m, 4H), 3.75-3.84 (m, 4H), 3.23-3.49 (m, 7H), 2.73-3.07 (m, 8H), 1.89-2.21 (m, 9H), 0.91-1.77 (m, 25H), 0.77-0.91 (m, 12H). MS (ESI) m/e 1496.3 (M+H)⁺.

### 1.33. Synthesis of N-[6-(2,5-dioxo-2,5-dihydro-1H-pyrrol-1-yl)hexanoyl]-L-valyl-N-[4-({[(4-{[2-({3-[(4-{6-[8-(1,3-benzothiazol-2-ylcarbamoyl)-3,4-dihydroisoquinolin-2(1H)-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](3-phosphonopropyl)amino}piperidin-1-yl)carbonyl]oxy}methyl)phenyl]-N⁵-carbamoyl-L-ornithinamide (Synthon HT)

A solution of Example 1.26.2 (0.040 g), 4-((S)-2-((S)-2-(6-(2,5-dioxo-2,5-dihydro-1H-pyrrol-1-yl)hexanamido)-3-methylbutanamido)-5-ureidopentanamido)benzyl (4-nitrophenyl) carbonate (0.030 g) and N,N-diisopropylethylamine (0.020 mL) in N,N-dimethylformamide (0.5 mL) was stirred at room temperature. After stirring for 3 hours, the reaction was diluted with N,N-dimethylformamide (1.25 mL) and water (0.5 mL). The mixture was purified by reverse phase HPLC using a Gilson system, eluting with 10-90% acetonitrile in water containing 0.1% v/v trifluoroacetic acid. The desired fractions were combined and freeze-dried to provide the title compound. ¹H NMR (500 MHz, dimethyl sulfoxide-d₆) δ ppm 9.98 (s, 1H), 9.26 (s, 1H), 8.06 (d, 1H), 8.05-8.01 (m, 1H), 7.79 (d, 2H), 7.62 (d, 1H), 7.61-7.57 (m, 2H), 7.52-7.42 (m, 3H), 7.38 (d, 1H), 7.35 (d, 1H), 7.32-7.26 (m, 3H), 6.99 (s, 2H), 6.95 (d, 1H), 6.01 (s, 1H), 4.99 (s, 2H), 4.96 (s, 3H), 4.44-4.33 (m, 2H), 4.18 (dd, 2H), 3.88 (t, 2H), 3.83 (s, 2H), 3.71-3.61 (m, 2H), 3.53 (t, 2H), 3.36 (t, 2H), 3.07-2.66 (m, 8H), 2.28-2.06 (m, 6H), 2.05-1.92 (m, 2H), 1.92-1.80 (m, 2H), 1.78-0.95 (m, 32H), 0.92-0.77 (m, 14H). MS (ESI) m/e 1549.5 (M+H)⁺.

### 1.34. Synthesis of N-[6-(2,5-dioxo-2,5-dihydro-1H-pyrrol-1-yl)hexanoyl]-L-valyl-N-{4-[({[2-({3-[(4-{6-[8-(1,3-benzothiazol-2-ylcarbamoyl)naphthalen-2-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](3-phosphonopropyl)carbamoyl}oxy)methyl]phenyl}-N⁵-carbamoyl-L-ornithinamide (Synthon HV)

The title compound was prepared by substituting Example 1.14.4 for Example 2.5.3 in Example 2.5.4. ¹H NMR (400 MHz, dimethyl sulfoxide-d₆) δ ppm 9.98 (s, 1H), 9.02 (s, 1H), 8.32-8.45 (m, 1H), 8.12-8.27 (m, 3H), 7.98-8.09 (m, 3H), 7.93 (d, 1H), 7.66-7.83 (m, 4H), 7.54-7.64 (m, 2H), 7.46-7.50 (m, 2H), 7.24-7.40 (m, 3H), 6.99 (s, 2H), 5.93-6.09 (m, 1H), 4.99 (s, 3H), 4.33-4.49 (m, 3H), 4.15-4.20 (m, 3H), 3.19-3.50 (m, 10H), 2.86-3.07 (m, 3H), 1.87-2.27 (m, 7H), 0.91-1.77 (m, 26H), 0.76-0.89 (m, 10H). MS (ESI) m/e 1461.1 (M+H)⁺.

### 1.35. Synthesis of N-[6-(2,5-dioxo-2,5-dihydro-1H-pyrrol-1-yl)hexanoyl]-L-valyl-N-[4-({[(4-{[2-({3-[(4-{6-[8-(1,3-benzothiazol-2-ylcarbamoyl)-3,4-dihydroisoquinolin-2(1H)-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](2-carboxyethyl)amino}piperidin-1-yl)carbonyl]oxy}methyl)phenyl]-N⁵-carbamoyl-L-ornithinamide (Synthon Hz)

A solution of Example 1.36.2 (0.031 g), 4-((S)-2-((S)-2-(6-(2,5-dioxo-2,5-dihydro-1H-pyrrol-1-yl)hexanamido)-3-methylbutanamido)-5-ureidopentanamido)benzyl (4-nitrophenyl) carbonate (0.025 g) and N,N-diisopropylethylamine (0.016 mL) in N,N-dimethylformamide (0.5 mL) was stirred at room temperature. After stirring for 3 hours, the reaction was diluted with N,N-dimethylformamide (1.25 mL) and water (0.5 mL). The mixture was purified by reverse phase HPLC using a Gilson system, eluting with 10-90% acetonitrile in water containing 0.1% v/v trifluoroacetic acid. The desired fractions were combined and freeze-dried to provide the title compound. ¹H NMR (400 MHz, dimethyl sulfoxide-d₆) δ ppm 12.84 (s, 1H), 9.98 (s, 1H), 8.82 (s, 1H), 8.05 (dd, 2H), 7.79 (d, 2H), 7.70-7.53 (m, 2H), 7.53-7.24 (m, 6H), 6.99 (s, 2H), 6.95 (d, 1H), 6.00 (s, 1H), 4.99 (s, 2H), 4.96 (s, 2H), 4.37 (q, 2H), 4.25-4.15 (m, 2H), 3.88 (t, 2H), 3.83 (s, 2H), 3.69-3.61 (m, 2H), 3.44-3.30 (m, 4H), 3.08-2.90 (m, 4H), 2.90-2.72 (m, 4H), 2.27-2.04 (m, 5H), 2.04-1.89 (m, 2H), 1.77-0.94 (m, 28H), 0.91-0.78 (m, 14H). MS (ESI) m/e 1499.5 (M+H)⁺.

### 1.36. Synthesis of N-[6-(2,5-dioxo-2,5-dihydro-1H-pyrrol-1-yl)hexanoyl]-L-valyl-N⁵-carbamoyl-N-{4-[({[2-({3-[(4-{2-carboxy-6-[8-([1,3]thiazolo[4,5-b]pyridin-2-ylcarbamoyl)-3,4-dihydroisoquinolin-2(1H)-yl]pyridin-3-yl}-5-methyl-1H-pyrazol-1-yl)methyl]-5,7-dimethyltricyclo[3.3.1.1^{3,7}]dec-1-yl}oxy)ethyl](3-phosphonopropyl)carbamoyl}oxy)methyl]phenyl}-L-ornithinamide (Synthon IA)

The title compound was prepared by substituting Example 1.39.2 for Example 1.2.9 in Example 2.1. ¹H NMR (400 MHz, dimethyl sulfoxide-d₆) δ ppm 9.98 (s, 1H), 8.60 (dd, 1H), 8.52 (dd, 1H), 8.06 (d, 1H), 7.78 (d, 1H), 7.65 (d, 1H), 7.59 (br m, 2H), 7.50 (m, 1H), 7.45 (d, 1H), 7.38 (m, 2H), 7.28 (s, 1H), 7.27 (d, 2H), 6.99 (s, 2H), 6.97 (d, 1H), 5.98 (br s, 1H), 4.98 (s, 4H), 4.39 (m, 1H), 4.19 (br m, 1H), 3.88 (t, 2H), 3.80 (br d, 2H), 3.36 (br m, 3H), 3.24 br (m, 4H), 2.98 (m, 4H), 2.16 (m, 2H), 2.12 (s, 3H), 1.95 (br m, 1H), 1.67 (br m, 3H), 1.34-1.47 (m, 9H), 1.08-1.23 (m, 11H), 0.95 (br m, 2H), 0.85-0.80 (m, 12H). MS (ESI) m/e 1465.5 (M−H)⁻.

### 1.37. Synthesis of N-[6-(2,5-dioxo-2,5-dihydro-1H-pyrrol-1-yl)hexanoyl]-L-valyl-N⁵-carbamoyl-N-{4-[({[2-({3-[(4-{2-carboxy-6-[8-([1,3]thiazolo[5,4-b]pyridin-2-ylcarbamoyl)-3,4-dihydroisoquinolin-2(1H)-yl]pyridin-3-yl}-5-methyl-1H-pyrazol-1-yl)methyl]-5,7-dimethyltricyclo[3.3.1.1^{3,7}]dec-1-yl}oxy)ethyl](3-phosphonopropyl)carbamoyl}oxy)methyl]phenyl}-L-ornithinamide (Synthon IF)

The title compound was prepared by substituting Example 1.40.2 for Example 1.2.9 in Example 2.1. ¹H NMR (400 MHz, dimethyl sulfoxide-d₆) δ ppm 9.98 (s, 1H), 8.52 (dd, 1H), 8.16 (dd, 1H), 8.05 (br d, 1H), 7.78 (br d, 1H), 7.62 (m, 1H), 7.58 (br m, 2H), 7.52 (m, 2H), 7.44 (d, 1H), 7.38 (t, 1H), 7.29 (s, 1H) 7.27 (d, 2H), 6.99 (s, 2H), 6.97 (d, 1H), 4.98 (s, 2H), 4.96 (s, 2H), 4.39 (m, 1H), 4.19 (br m, 1H), 3.88 (t, 2H), 3.80 (br d, 2H), 3.36 (br m, 3H), 3.24 br (m, 4H), 2.98 (m, 4H), 2.16 (m, 2H), 2.12 (s, 3H), 1.95 (br m, 1H), 1.67 (br m, 3H), 1.47-1.34 (m, 9H), 1.08-1.23 (m, 11H), 0.95 (br m, 2H), 0.85-0.80 (m, 12H). MS (ESI) m/e 1451.5 (M−H)⁻.

### 1.38. Synthesis of N-{6-[(chloroacetyl)amino]hexanoyl}-L-valyl-N-{4-[({[2-({3-[(4-{6-[8-(1,3-benzothiazol-2-ylcarbamoyl)-3,4-dihydroisoquinolin-2(1H)-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](2-sulfoethyl)carbamoyl}oxy)methyl]phenyl}-L-alaninamide (Synthon IG)

### 1.38.1. 3-(1-((3-(2-((((4-((S)-2-((S)-2-amino-3-methylbutanamido)propanamido)benzyl)oxy)carbonyl)(2-sulfoethyl)amino)ethoxy)-5,7-dimethyladamantan-1-yl)methyl)-5-methyl-1H-pyrazol-4-yl)-6-(8-(benzo[d]thiazol-2-ylcarbamoyl)-3,4-dihydroisoquinolin-2(1H)-yl)picolinic Acid

A solution of Example 1.2.9 (0.050 g), (9H-fluoren-9-yl)methyl ((S)-3-methyl-1-(((S)-1-((4-((((4-nitrophenoxy)carbonyl)oxy)methyl)phenyl)amino)-1-oxopropan-2-yl)amino)-1-oxobutan-2-yl)carbamate (0.039 g) and N,N-diisopropylethylamine (0.027 mL) in N,N-dimethylformamide (1 mL) was stirred at room temperature. After stirring overnight, diethylamine (0.027 mL) was added to the reaction, and stirring was continued for 2 hours. The reaction was quenched with trifluoroacetic acid, and the mixture was purified by reverse phase HPLC using a Gilson system, eluting with 5-75% acetonitrile in water containing 0.1% v/v trifluoroacetic acid. The desired fractions were combined and freeze-dried to provide the title compound. MS (ESI) m/e 1499.5 (M+H)⁺.

### 1.38.2. N-{6-[(chloroacetyl)amino]hexanoyl}-L-valyl-N-{4-[({[2-({3-[(4-{6-[8-(1,3-benzothiazol-2-ylcarbamoyl)-3,4-dihydroisoquinolin-2(1H)-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](2-sulfoethyl)carbamoyl}oxy)methyl]phenyl}-L-alaninamide

To a solution of 6-(2-chloroacetamido)hexanoic acid (6 mg) and 2-(3H-[1,2,3]triazolo[4,5-b]pyridin-3-yl)-1,1,3,3-tetramethylisouronium hexafluorophosphate(V) (0.011 g) in N,N-dimethylformamide (1 mL) was added N,N-diisopropylethylamine (0.015 mL), and the reaction stirred for 5 minutes. This solution was added to Example 2.38.1 (0.022 g) and was stirred for 1 hour. The reaction was diluted with N,N-dimethylformamide (1 mL) and water (0.5 mL). The mixture was purified by reverse phase HPLC using a Gilson system, eluting with 10-90% acetonitrile in water containing 0.1% v/v trifluoroacetic acid. The desired fractions were combined and freeze-dried to provide the title compound. ¹H NMR (400 MHz, dimethyl sulfoxide-d₆) δ ppm 12.83 (s, 1H), 9.93 (s, 1H), 8.20-8.10 (m, 2H), 8.04 (d, 1H), 7.83-7.76 (m, 2H), 7.64-7.55 (m, 3H), 7.55-7.50 (m, 1H), 7.50-7.41 (m, 2H), 7.40-7.32 (m, 2H), 7.32-7.24 (m, 3H), 6.96 (d, 1H), 5.07-4.92 (m, 3H), 4.39 (p, 1H), 4.18 (dd, 2H), 4.01 (s, 2H), 3.92-3.76 (m, 6H), 3.54-3.32 (m, 4H), 3.25 (t, 2H), 3.13-2.93 (m, 4H), 2.72-2.58 (m, 2H), 2.29-2.12 (m, 2H), 2.09 (s, 3H), 2.05-1.92 (m, 1H), 1.58-0.89 (m, 18H), 0.89-0.77 (m, 12H). MS (ESI) m/e 1362.2 (M+H)⁺.

### 1.39. Synthesis of N-[6-(2,5-dioxo-2,5-dihydro-1H-pyrrol-1-yl)hexanoyl]-L-valyl-N-{4-[({[2-({3-[(4-{6-[8-(1,3-benzothiazol-2-ylcarbamoyl)-5-(carboxymethoxy)-3,4-dihydroisoquinolin-2(1H)-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](methyl)carbamoyl}oxy)methyl]phenyl}-N⁵-carbamoyl-L-ornithinamide (Synthon IJ)

The title compound was prepared by substituting Example 1.41.3 for Example 2.5.3 in Example 2.5.4. ¹H NMR (400 MHz, dimethyl sulfoxide-d₆) δ ppm 10.03 (s, 1H), 9.96 (s, 1H), 8.26-8.34 (m, 1H), 7.95-8.11 (m, 2H), 7.73-7.82 (m, 2H), 7.22-7.70 (m, 11H), 6.95-7.05 (m, 3H), 6.89 (d, 1H), 5.23 (s, 1H), 4.98 (d, 3H), 4.83 (s, 1H), 4.33-4.43 (m, 1H), 4.11-4.23 (m, 1H), 3.74-3.95 (m, 3H), 3.22-3.39 (m, 10H), 2.78-3.06 (m, 12H), 1.91-2.22 (m, 8H), 0.93-1.68 (m, 20H), 0.77-0.88 (m, 10H). MS (ESI) m/e 1432.2 (M+H)⁺.

### 1.40. Synthesis of N-[6-(2,5-dioxo-2,5-dihydro-1H-pyrrol-1-yl)hexanoyl]-L-valyl-N-[4-({[(2-{[2-({3-[(4-{6-[8-(1,3-benzothiazol-2-ylcarbamoyl)-3,4-dihydroisoquinolin-2(1H)-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](2-sulfoethyl)amino}ethyl)(2-carboxyethyl)carbamoyl]oxy}methyl)phenyl]-N⁵-carbamoyl-L-ornithinamide (Synthon U)

The title compound was prepared as described in Example 2.1, replacing Example 1.2.9 with Example 1.38.2. ¹H NMR (400 MHz, dimethyl sulfoxide-d₆) δ ppm 12.86 (s, 1H), 9.99 (s, 1H), 9.10 (s, 1H), 8.04 (t, 2H), 7.73-7.85 (m, 2H), 7.61 (t, 3H), 7.41-7.55 (m, 3H), 7.26-7.39 (m, 5H), 6.99 (s, 2H), 6.95 (d, 1H), 6.00 (s, 1H), 4.99 (d, 4H), 4.34-4.45 (m, 2H), 4.19 (dd, 2H), 3.88 (t, 2H), 3.82 (s, 2H), 3.36 (t, 4H), 2.85-3.09 (m, 5H), 2.06-2.22 (m, 4H), 1.89-2.02 (m, 1H), 0.94-1.77 (m, 20H), 0.77-0.90 (m, 11H). MS (ESI) m/e 1567.4 (M+H)⁺.

### 1.41. Synthesis of 6-[8-(1,3-benzothiazol-2-ylcarbamoyl)-3,4-dihydroisoquinolin-2(1H)-yl]-3-[1-({3-[2-({(2S)-2-[{[(4-{[(2S)-5-(carbamoylamino)-2-{[(2S)-2-{[6-(2,5-dioxo-2,5-dihydro-1H-pyrrol-1-yl)hexanoyl]amino}-3-methylbutanoyl]amino}pentanoyl]amino}benzyl)oxy]carbonyl}(2-carboxyethyl)amino]-3-carboxypropanoyl}amino)ethoxy]-5,7-dimethyltricyclo[3.3.1.1^{3,7}]dec-1-yl}methyl)-5-methyl-1H-pyrazol-4-yl]pyridine-2-carboxylic Acid (Synthon IK)

The title compound was prepared as described in Example 2.1, replacing Example 1.2.9 with Example 1.32.4. MS (ESI) m/e 1592.4 (M−H)⁻.

### 1.42. Synthesis of 6-[8-(1,3-benzothiazol-2-ylcarbamoyl)-3,4-dihydroisoquinolin-2(1H)-yl]-3-(1-{[3-(2-{[(2S)-2-({[(4-{[(2S)-5-(carbamoylamino)-2-{[(2S)-2-{[6-(2,5-dioxo-2,5-dihydro-1H-pyrrol-1-yl)hexanoyl]amino}-3-methylbutanoyl]amino}pentanoyl]amino}benzyl)oxy]carbonyl}amino)-3-carboxypropanoyl](2-sulfoethyl)amino}ethoxy)-5,7-dimethyltricyclo[3.3.1.1^{3,7}]dec-1-yl]methyl}-5-methyl-1H-pyrazol-4-yl)pyridine-2-carboxylic Acid (Synthon IL)

The title compound was prepared as described in Example 2.1, replacing Example 1.2.9 with Example 1.44.2. ¹H NMR (400 MHz, dimethyl sulfoxide-d₆) δ ppm 12.82 (s, 1H), 9.96 (s, 1H), 8.03 (t, 2H), 7.77 (d, 2H), 7.39-7.62 (m, 7H), 7.30-7.39 (m, 2H), 7.22-7.29 (m, 3H), 6.98 (s, 2H), 6.92-6.96 (m, 1H), 5.97 (s, 1H), 4.83-5.05 (m, 3H), 3.83-3.92 (m, 1H), 3.79 (s, 1H), 3.00 (s, 2H), 2.03-2.22 (m, 8H), 1.94 (s, 2H), 1.34 (d, 30H), 0.69-0.90 (m, 13H). MS (ESI) m/e 1565.5 (M−H)⁻.

### 1.43. Synthesis of N-[6-(2,5-dioxo-2,5-dihydro-1H-pyrrol-1-yl)hexanoyl]-L-valyl-N-[4-({[(4-{[2-({3-[(4-{6-[8-(1,3-benzothiazol-2-ylcarbamoyl)-3,4-dihydroisoquinolin-2(1H)-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](3-carboxypropyl)amino}piperidin-1-yl)carbonyl]oxy}methyl)phenyl]-N⁵-carbamoyl-L-ornithinamide (Synthon IM)

A solution of Example 1.42.2 (0.045 g), 4-((S)-2-((S)-2-(6-(2,5-dioxo-2,5-dihydro-1H-pyrrol-1-yl)hexanamido)-3-methylbutanamido)-5-ureidopentanamido)benzyl (4-nitrophenyl) carbonate (0.035 g) and N,N-diisopropylethylamine (0.038 mL) in N,N-dimethylformamide (0.5 mL) was stirred at room temperature. After stirring for 3 hours, the reaction was diluted with N,N-dimethylformamide (1.25 mL) and water (0.5 mL). The mixture was purified by reverse phase HPLC using a Gilson system, eluting with 10-90% acetonitrile in water containing 0.1% v/v trifluoroacetic acid. The desired fractions were combined and freeze-dried to provide the title compound. ¹H NMR (400 MHz, dimethyl sulfoxide-d₆) δ ppm 12.76 (s, 1H), 9.91 (s, 1H), 8.79 (s, 1H), 7.98 (dd, 2H), 7.72 (d, 2H), 7.68-7.47 (m, 3H), 7.47-7.00 (m, 7H), 6.96-6.83 (m, 3H), 5.93 (s, 1H), 4.91 (d, 3H), 4.30 (q, 1H), 4.17-3.97 (m, 4H), 3.96-3.53 (m, 4H), 3.34-2.65 (m, 12H), 2.25 (t, 2H), 2.16-1.67 (m, 12H), 1.67-0.88 (m, 26H), 0.84-0.70 (m, 12H). MS (ESI) m/e 1513.6 (M+H)⁺.

### 1.44. Synthesis of 4-{[(1E)-{3-({[2-({3-[(4-{6-[8-(1,3-benzothiazol-2-ylcarbamoyl)-5-(carboxymethoxy)-3,4-dihydroisoquinolin-2(1H)-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](methyl)carbamoyl}oxy)prop-1-en-1-yl]-2-(}N-[6-(2,5-dioxo-2,5-dihydro-H-pyrrol-1-yl)hexanoyl]-beta-alanyl}amino)phenyl beta-D-glucopyranosiduronic Acid (Synthon IO)

### 1.44.1. (E)-tert-butyldimethyl((3-(4,4,5,5-tetramethyl-1,3,2-dioxaborolan-2-yl)allyl)oxy)silane

To a flask charged with tert-butyldimethyl(prop-2-yn-1-yloxy)silane (5 g) and dichloromethane (14.7 mL) under nitrogen atmosphere was added dropwise 4,4,5,5-tetramethyl-1,3,2-dioxaborolane (3.94 g). The mixture was stirred at room temperature for one minute then transferred via cannula to a nitrogen-sparged flask containing Cp₂ZrClH (chloridobis(η5-cyclopentadienyl)hydridozirconium, Schwartz's Reagent) (379 mg). The resulting reaction mixture was stirred at room temperature for 16 hours. The mixture was carefully quenched with water (15 mL), and then extracted with diethyl ether (3×30 mL). The combined organic phases were washed with water (15 mL), dried over MgSO₄, filtered, and purified by silica gel chromatography, eluting with a gradient from 0-8% ethyl acetate/heptanes to give the title compound. MS (ESI) m/z 316.0 (M+NH₄)⁺.

### 1.44.2. (2S,3R,4S,5S,6S)-2-(4-bromo-2-nitrophenoxy)-6-(methoxycarbonyl)tetrahydro-2H-pyran-3,4,5-triyl Triacetate

(2R,3R,4S,5S,6S)-2-Bromo-6-(methoxycarbonyl)tetrahydro-2H-pyran-3,4,5-triyl triacetate (5 g) was dissolved in acetonitrile (100 mL). Ag₂O (2.92 g) was added to the solution, and the reaction was stirred for 5 minutes at room temperature. 4-Bromo-2-nitrophenol (2.74 g) was added, and the reaction mixture was stirred at room temperature for 4 hours. The silver salt residue was filtered through diatomaceous earth, and the filtrate was concentrated under reduced pressure. The residue was purified by silica gel chromatography, eluting with a gradient of 10-70% ethyl acetate in heptanes, to give the title compound. MS (ESI+) m/z 550.9 (M+NH₄)⁺.

### 1.44.3. (2S,3R,4S,5S,6S)-2-(4-((E)-3-((tert-butyldimethylsilyl)oxy)prop-1-en-1-yl)-2-nitrophenoxy)-6-(methoxycarbonyl)tetrahydro-2H-pyran-3,4,5-triyl Triacetate

Example 2.44.2 (1 g), sodium carbonate (0.595 g), tris(dibenzylideneacetone)dipalladium (Pd₂(dba)₃) (0.086 g), and 1,3,5,7-tetramethyl-6-phenyl-2,4,8-trioxa-6-phosphaadamantane (0.055 g) were combined in a 3-neck 50-mL round bottom flask equipped with a reflux condenser and the system was degassed with nitrogen. Separately, a solution of Example 2.44.1 (0.726 g) in tetrahydrofuran (15 mL) was degassed with nitrogen for 30 minutes. The latter solution was transferred via cannula into the flask containing the solid reagents, followed by addition of degassed water (3 mL) via syringe. The reaction was heated to 60° C. for two hours. The reaction mixture was partitioned between ethyl acetate (3×30 mL) and water (30 mL). The combined organic phases were dried (Na₂SO₄), filtered, and concentrated. The residue was purified by silica gel chromatography, eluting with a gradient from 0-35% ethyl acetate in heptanes, to provide the title compound. MS (ESI+) m/z 643.1 (M+NH₄)⁺.

### 1.44.4. (2S,3R,4S,5S,6S)-2-(2-amino-4-((E)-3-hydroxyprop-1-en-1-yl)phenoxy)-6-(methoxycarbonyl)tetrahydro-2H-pyran-3,4,5-triyl Triacetate

A 500-mL three-neck, nitrogen-flushed flask equipped with a pressure-equalizing addition funnel was charged with zinc dust (8.77 g). A degassed solution of Example 2.44.3 (8.39 g) in tetrahydrofuran (67 mL) was added via cannula. The resulting suspension was chilled in an ice bath, and 6N HCl (22.3 mL) was added dropwise via the addition funnel at such a rate that the internal temperature of the reaction did not exceed 35° C. After the addition was complete, the reaction was stirred for two hours at room temperature, and filtered through a pad of diatomaceous earth, rinsing with water and ethyl acetate. The filtrate was treated with saturated aqueous NaHCO₃ solution until the water layer was no longer acidic, and the mixture was filtered to remove the resulting solids. The filtrate was transferred to a separatory funnel, and the layers were separated. The aqueous layer was extracted with ethyl acetate (3×75 mL), and the combined organic layers were washed with water (100 mL), dried over Na₂SO₄, filtered, and concentrated. The residue was triturated with diethyl ether and the solid collected by filtration to provide the title compound. MS (ESI+) m/z 482.0 (M+H)⁺.

### 1.44.5. (9H-fluoren-9-yl)methyl (3-chloro-3-oxopropyl)carbamate

To a solution of 3-((((9H-fluoren-9-yl)methoxy)carbonyl)amino)propanoic acid (5.0 g) in dichloromethane (53.5 mL) was added sulfurous dichloride (0.703 mL). The mixture was stirred at 60° C. for one hour. The mixture was cooled and concentrated to give the title compound, which was used in the next step without further purification.

### 1.44.6. (2S,3R,4S,5S,6S)-2-(2-(3-((((9H-fluoren-9-yl)methoxy)carbonyl)amino)propanamido)-4-((E)-3-hydroxyprop-1-en-1-yl)phenoxy)-6-(methoxycarbonyl)tetrahydro-2H-pyran-3,4,5-triyl Triacetate

Example 2.44.4 (6.78 g) was dissolved in dichloromethane (50 mL), and the solution was chilled to 0° C. in an ice bath. N,N-Diisopropylethylamine (3.64 g) was added, followed by dropwise addition of a solution of Example 2.44.5 (4.88 g) in dichloromethane (50 mL). The reaction was stirred for 16 hours allowing the ice bath to come to room temperature. Saturated aqueous NaHCO₃ solution (100 mL) was added, and the layers were separated. The aqueous layer was further extracted with dichloromethane (2×50 mL). The extracts were dried over Na₂SO₄, filtered, concentrated and purified by silica gel chromatography, eluting with a gradient of 5-95% ethyl acetate/heptane, to give an inseparable mixture of starting aniline and desired product. The mixture was partitioned between 1N aqueous HCl (40 mL) and a 1:1 mixture of diethyl ether and ethyl acetate (40 mL), and then the aqueous phase was further extracted with ethyl acetate (2×25 mL). The organic phases were combined, washed with water (2×25 mL), dried over Na₂SO₄, filtered, and concentrated to give the title compound. MS (ESI+) m/z 774.9 (M+H)⁺.

### 1.44.7. (2S,3R,4S,5S,6S)-2-(2-(3-((((9H-fluoren-9-yl)methoxy)carbonyl)amino)propanamido)-4-((E)-3-(((4-nitrophenoxy)carbonyl)oxy)prop-1-en-1-yl)phenoxy)-6-(methoxycarbonyl)tetrahydro-2H-pyran-3,4,5-triyl Triacetate

Example 2.44.6 (3.57 g) was dissolved in dichloromethane (45 mL) and bis(4-nitrophenyl)carbonate (2.80 g) was added, followed by dropwise addition of N,N-diisopropylethylamine (0.896 g). The reaction mixture was stirred at room temperature for two hours. Silica gel (20 g) was added to the reaction solution, and the mixture was concentrated to dryness under reduced pressure, keeping the bath temperature at or below 25° C. The silica residue was loaded atop a column, and the product was purified by silica gel chromatography, eluting with a gradient from 0-100% ethyl acetate-heptane, providing partially purified product which was contaminated with nitrophenol. The material was triturated with methyl tert-butyl ether (250 mL), and the resulting slurry was allowed to sit for 1 hour. The product was collected by filtration. Three successive crops were collected in a similar fashion to give the title compound. MS (ESI+) m/z 939.8 (M+H)⁺.

### 1.44.8. 3-(1-((3-(2-(((((E)-3-(3-(3-aminopropanamido)-4-(((2S,3R,4S,5S,6S)-6-carboxy-3,4,5-trihydroxytetrahydro-2H-pyran-2-yl)oxy)phenyl)allyl)oxy)carbonyl)methyl)amino)ethoxy)-5,7-dimethyladamantan-1-yl)methyl)-5-methyl-1H-pyrazol-4-yl)-6-(8-(benzo[d]thiazol-2-ylcarbamoyl)-5-(carboxymethoxy)-3,4-dihydroisoquinolin-2(1H)-yl)picolinic Acid

To a cold (0° C.) solution of Example 2.44.7 (19.7 mg) and Example 1.41.3 (18.5 mg) in N,N-dimethylformamide (2 mL) was added N,N-diisopropylethylamine (0.054 mL). The reaction was slowly warmed to room temperature and stirred overnight. To the reaction mixture was added water (2 mL) and lithium hydroxide monohydrate (50 mg), and the mixture was stirred overnight. The mixture was acidified with trifluoroacetic acid and filtered. The mixture was purified by reverse phase HPLC (Gilson system), eluting with 10-85% acetonitrile in 0.1% trifluoroacetic acid in water, to provide the title compound. MS (ESI) m/e 1273.2 (M+H)⁺.

### 1.44.9. 4-[(1E)-3-({[2-({3-[(4-{6-[8-(1,3-benzothiazol-2-ylcarbamoyl)-5-(carboxymethoxy)-3,4-dihydroisoquinolin-2(1H)-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](methyl)carbamoyl}oxy)prop-1-en-1-yl]-2-({N-[6-(2,5-dioxo-2,5-dihydro-1H-pyrrol-1-yl)hexanoyl]-beta-alanyl}amino)phenyl Beta-D-Glucopyranosiduronic Acid

To a solution of Example 2.44.8 (10 mg) and 2,5-dioxopyrrolidin-1-yl 6-(2,5-dioxo-2,5-dihydro-1H-pyrrol-1-yl)hexanoate (2.3 mg) in N,N-dimethylformamide (2 mL) was added N,N-diisopropylethylamine (0.054 mL). The reaction was stirred overnight. The reaction mixture was diluted with methanol (2 mL) and acidified with trifluoroacetic acid. The mixture was purified by reverse phase HPLC (Gilson system), eluting with 10-85% acetonitrile in 0.1% trifluoroacetic acid in water, to give the title compound. ¹H NMR (400 MHz, dimethyl sulfoxide-d₆) δ ppm 12.70 (s, 1H), 9.03 (s, 1H), 8.25 (s, 1H), 8.01 (d, 1H), 7.87 (t, 1H), 7.77 (d, 1H), 7.69 (d, 1H), 7.41-7.55 (m, 2H), 7.23-7.38 (m, 2H), 6.79-7.16 (m, 7H), 6.56 (d, 1H), 6.09-6.25 (m, 1H), 4.96-5.07 (m, 3H), 4.84 (s, 3H), 4.64 (d, 3H), 3.87-3.97 (m, 5H), 3.24-3.47 (m, 12H), 2.77-2.95 (m, 6H), 1.94-2.08 (m, 6H), 0.92-1.56 (m, 20H), 0.74-0.86 (m, 6H). MS (ESI) m/e 1487.3 (M+Na)⁺.

### 1.45. Synthesis of N-[6-(2,5-dioxo-2,5-dihydro-1H-pyrrol-1-yl)hexanoyl]-L-valyl-N-{4-[({[2-({3-[(4-{6-[8-(1,3-benzothiazol-2-ylcarbamoyl)naphthalen-2-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](2-sulfoethyl)carbamoyl}oxy)methyl]phenyl}-N⁵-carbamoyl-L-ornithinamide (Synthon IP)

The title compound was prepared by substituting Example 1.43.7 for Example 2.5.3 in Example 2.5.4. ¹H NMR (400 MHz, dimethyl sulfoxide-d₆) δ ppm 13.09 (s, 1H), 9.99 (s, 1H), 9.02 (s, 1H), 8.30-8.40 (m, 3H), 7.93-8.25 (m, 6H), 7.23-7.86 (m, 10H), 6.92-7.05 (m, 2H), 4.99 (d, 2H), 4.36-4.44 (m, 2H), 4.14-4.23 (m, 2H), 2.87-3.35 (m, 12H), 2.81 (t, 2H), 2.59-2.70 (m, 2H), 1.84-2.28 (m, 8H), 0.97-1.77 (m, 20H), 0.77-0.88 (m, 10H). MS (ESI) m/e 1448.3 (M+Na)⁺.

### 1.46. Synthesis of N-[6-(2,5-dioxo-2,5-dihydro-1H-pyrrol-1-yl)hexanoyl]-L-valyl-N-[4-({[(2-{[8-(1,3-benzothiazol-2-ylcarbamoyl)-2-(6-carboxy-5-{1-[(3,5-dimethyl-7-{2-[methyl(2-sulfoethyl)amino]ethoxy}tricyclo[3.3.1.1^{3,7}]dec-1-yl)methyl]-5-methyl-1H-pyrazol-4-yl}pyridin-2-yl)-1,2,3,4-tetrahydroisoquinolin-5-yl]oxy}ethyl)carbamoyl]oxy}methyl)phenyl]-N⁵-carbamoyl-L-ornithinamide (Synthon IS)

The title compound was prepared as described in Example 2.1, replacing Example 1.2.9 with Example 1.46.2. ¹H NMR (400 MHz, dimethyl sulfoxide-d₆) δ ppm 12.69 (s, 1H), 9.97 (s, 1H), 8.97 (s, 1H), 8.04 (dd, 2H), 7.78 (d, 2H), 7.71 (d, 1H), 7.59 (d, 2H), 7.44-7.54 (m, 3H), 7.26-7.37 (m, 4H), 6.96-7.03 (m, 4H), 5.97 (s, 1H), 4.99 (d, 4H), 4.31-4.45 (m, 1H), 4.18 (dd, 1H), 4.09 (s, 2H), 3.85-3.93 (m, 2H), 3.83 (s, 2H), 3.39-3.47 (m, 2H), 3.24-3.39 (m, 4H), 3.12-3.24 (m, 2H), 2.75-3.07 (m, 9H), 2.06-2.23 (m, 5H), 1.90-2.01 (m, 1H), 1.54-1.75 (m, 2H), 1.24-1.52 (m, 12H), 0.91-1.24 (m, 8H), 0.77-0.88 (m, 12H). MS (ESI) m/e 1525.4 (M+H)⁺.

### 1.47. Synthesis of N-[6-(2,5-dioxo-2,5-dihydro-1H-pyrrol-1-yl)hexanoyl]-L-valyl-N-[4-({[(2-{[8-(1,3-benzothiazol-2-ylcarbamoyl)-2-(6-carboxy-5-{1-[(3,5-dimethyl-7-{2-[methyl(2-sulfoethyl)amino]ethoxy}tricyclo[3.3.1.1^{3,7}]dec-1-yl)methyl]-5-methyl-1H-pyrazol-4-yl}pyridin-2-yl)-1,2,3,4-tetrahydroisoquinolin-5-yl]oxy}ethyl)(2-sulfoethyl)carbamoyl]oxy}methyl)phenyl]-N⁵-carbamoyl-L-ornithinamide (Synthon IU)

The title compound was prepared as described in Example 2.1, replacing Example 1.2.9 with Example 1.47.2. ¹H NMR (400 MHz, dimethyl sulfoxide-d₆) δ ppm 12.70 (s, 1H), 9.99 (s, 1H), 8.97 (s, 1H), 8.04 (dd, 2H), 7.78 (d, 2H), 7.71 (d, 1H), 7.59 (d, 2H), 7.43-7.55 (m, 2H), 7.28-7.37 (m, 4H), 6.94-7.07 (m, 4H), 6.05 (s, 1H), 4.93-5.11 (m, 4H), 4.31-4.46 (m, 2H), 4.12-4.26 (m, 4H), 3.80-3.95 (m, 4H), 3.40-3.50 (m, 2H), 3.24-3.40 (m, 6H), 3.13-3.24 (m, 2H), 2.74-3.08 (m, 9H), 2.63-2.73 (m, 2H), 2.05-2.23 (m, 5H), 1.96 (s, 1H), 1.52-1.77 (m, 2H), 1.23-1.53 (m, 12H), 0.97-1.22 (m, 8H), 0.77-0.89 (m, 12H). MS (ESI) m/e 1631.5 (M−H)⁻.

### 1.48. Synthesis of N-[6-(2,5-dioxo-2,5-dihydro-1H-pyrrol-1-yl)hexanoyl]-L-valyl-N-[4-({[(2-{([2-({3-[(4-{6-[8-(1,3-benzothiazol-2-ylcarbamoyl)-3,4-dihydroisoquinolin-2(1H)-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](2-sulfoethyl)amino}ethyl)(2-sulfoethyl)carbamoyl]oxy}methyl)phenyl]-N⁵-carbamoyl-L-ornithinamide (Synthon IV)

The title compound was prepared as described in Example 2.1, replacing Example 1.2.9 with Example 1.48.2. ¹H NMR (400 MHz, dimethyl sulfoxide-d₆) δ ppm 12.82 (s, 1H), 10.00 (s, 1H), 9.29-9.57 (m, 1H), 8.05 (t, 2H), 7.79 (d, 2H), 7.51-7.63 (m, 4H), 7.40-7.50 (m, 2H), 7.27-7.39 (m, 5H), 6.93-7.02 (m, 3H), 4.99 (d, 3H), 4.30-4.47 (m, 1H), 4.19 (t, 1H), 3.79-3.92 (m, 3H), 3.60-3.74 (m, 2H), 3.01 (s, 9H), 2.70 (d, 4H), 2.05-2.23 (m, 6H), 1.96 (d, 2H), 1.53-1.78 (m, 3H), 1.22-1.54 (m, 13H), 0.89-1.22 (m, 9H), 0.75-0.89 (m, 13H). MS (ESI) m/e 1603.3 (M+H)⁺.

### 1.49. Synthesis of N-{6-[(chloroacetyl)amino]hexanoyl}-L-valyl-N-{4-[({[2-({3-[(4-{6-[8-(1,3-benzothiazol-2-ylcarbamoyl)-3,4-dihydroisoquinolin-2(1H)-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](2-sulfoethyl)carbamoyl}oxy)methyl]phenyl}-N⁵-carbamoyl-L-ornithinamide (Synthon IZ)

### 1.49.1. 3-(1-(((1r,3r)-3-(2-((((4-((S)-2-((S)-2-amino-3-methylbutanamido)-5-ureidopentanamido)benzyl)oxy)carbonyl)(2-sulfoethyl)amino)ethoxy)-5,7-dimethyladamantan-1-yl)methyl)-5-methyl-1H-pyrazol-4-yl)-6-(8-(benzo[d]thiazol-2-ylcarbamoyl)-3,4-dihydroisoquinolin-2(1H)-yl)picolinic Acid

A solution of Example 1.2.9 (0.045 g) (9H-fluoren-9-yl)methyl ((S)-3-methyl-1-(((S)-1-((4-((((4-nitrophenoxy)carbonyl)oxy)methyl)phenyl)amino)-1-oxo-5-ureidopentan-2-yl)amino)-1-oxobutan-2-yl)carbamate (0.043 g) and N,N-diisopropylethylamine (0.041 mL) were stirred together in N,N-dimethylformamide (1 mL) at room temperature. After stirring overnight, diethylamine (0.024 mL) was added to the reaction, and stirring was continued for 2 hours. The reaction was quenched with trifluoroacetic acid then purified by reverse phase HPLC using a Gilson system, eluting with 10-75% acetonitrile in water containing 0.1% v/v trifluoroacetic acid. The desired fractions were combined and freeze-dried to provide the title compound.

### 1.49.2. N-{6-[(chloroacetyl)amino]hexanoyl}-L-valyl-N-{4-[({[2-({3-[(4-{6-[8-(1,3-benzothiazol-2-ylcarbamoyl)-3,4-dihydroisoquinolin-2(1H)-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](2-sulfoethyl)carbamoyl}oxy)methyl]phenyl}-N⁵-carbamoyl-L-ornithinamide

A solution of 6-(2-chloroacetamido)hexanoic acid (6.43 mg) and 2-(3H-[1,2,3]triazolo[4,5-b]pyridin-3-yl)-1,1,3,3-tetramethylisouronium hexafluorophosphate(V) (0.012 g) in N,N-dimethylformamide (0.5 mL) was added N,N-diisopropylethylamine (0.019 mL), and the reaction stirred for 5 minutes. This solution was added to Example 2.49.1 (0.026 g) and was stirred for 1 hour. The reaction was diluted with N,N-dimethylformamide (1 mL) and water (0.5 mL). The mixture was purified by reverse phase HPLC using a Gilson system, eluting with 10-60% acetonitrile in water containing 0.1% v/v trifluoroacetic acid. The desired fractions were combined and freeze-dried to provide the title compound. ¹H NMR (500 MHz, dimethyl sulfoxide-d₆) δ ppm 12.85 (s, 1H), 9.99 (s, 1H), 8.18 (q, 1H), 8.08 (d, 1H), 8.04 (d, 1H), 7.84-7.76 (m, 2H), 7.64-7.56 (m, 3H), 7.56-7.50 (m, 1H), 7.47 (t, 1H), 7.43 (d, 1H), 7.37 (d, 1H), 7.35 (d, 1H), 7.29 (s, 1H), 7.27 (d, 2H), 6.95 (d, 1H), 6.05 (s, 1H), 5.05-4.91 (m, 4H), 4.48-4.33 (m, 1H), 4.26-4.14 (m, 1H), 4.02 (s, 2H), 3.88 (t, 2H), 3.81 (d, 2H), 3.25 (t, 2H), 3.14-2.98 (m, 6H), 2.98-2.87 (m, 2H), 2.74-2.59 (m, 2H), 2.27-2.05 (m, 6H), 2.04-1.92 (m, 1H), 1.78-1.65 (m, 1H), 1.65-1.53 (m, 1H), 1.53-0.90 (m, 22H), 0.90-0.73 (m, 12H). MS (ESI) m/e 1448.2 (M+H)⁺.

### 1.50. Synthesis of N-[6-(2,5-dioxo-2,5-dihydro-1H-pyrrol-1-yl)hexanoyl]-L-valyl-N-{4-[({[2-({3-[(4-{6-[4-(1,3-benzothiazol-2-ylcarbamoyl)-3,4-dihydro-2H-1,4-benzoxazin-6-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](2-sulfoethyl)carbamoyl}oxy)methyl]phenyl}-N⁵-carbamoyl-L-ornithinamide (Synthon JD)

The title compound was prepared by substituting Example 1.51.8 for Example 2.5.3 in Example 2.5.4. ¹H NMR (400 MHz, dimethyl sulfoxide-d₆) δ ppm 9.56 (s, 1H), 8.51-8.59 (m, 1H), 7.89 (d, 1H), 7.82 (d, 1H), 7.69-7.77 (m, 2H), 7.34-7.62 (m, 7H), 7.16-7.34 (m, 4H), 6.95 (dd, 1H), 5.95-6.05 (m, 1H), 4.95 (s, 2H), 4.06-4.44 (m, 6H), 3.85 (s, 3H), 3.39-3.59 (m, 7H), 2.61-2.74 (m, 3H), 2.19 (s, 3H), 1.88-2.16 (m, 3H), 0.96-1.75 (m, 22H), 0.71-0.89 (m, 13H). MS (ESI) m/e 1454.2 (M+Na)⁺.

### 1.51. Synthesis of N-[6-(2,5-dioxo-2,5-dihydro-1H-pyrrol-1-yl)hexanoyl]-L-valyl-N-[4-({[(2-{[8-(1,3-benzothiazol-2-ylcarbamoyl)-2-(6-carboxy-5-{1-[(3,5-dimethyl-7-{2-[methyl(2-sulfoethyl)amino]ethoxy}tricyclo[3.3.1.1^{3,7}]dec-1-yl)methyl]-5-methyl-1H-pyrazol-4-yl}pyridin-2-yl)-1,2,3,4-tetrahydroisoquinolin-5-yl]oxy}ethyl)(2-carboxyethyl)carbamoyl]oxy}methyl)phenyl]-N⁵-carbamoyl-L-ornithinamide (Synthon JF)

The title compound was prepared as described in Example 2.1, replacing Example 1.2.9 with Example 1.49.2. ¹H NMR (500 MHz, dimethyl sulfoxide-d₆) δ ppm 12.71 (s, 1H), 10.00 (s, 1H), 8.97 (s, 1H), 8.08 (d, 1H), 8.02 (d, 1H), 7.78 (d, 2H), 7.72 (d, 1H), 7.60 (d, 2H), 7.52 (d, 1H), 7.44-7.50 (m, 1H), 7.27-7.39 (m, 4H), 6.96-7.06 (m, 3H), 5.98 (s, 1H), 5.01 (d, 4H), 4.31-4.46 (m, 1H), 4.18 (s, 3H), 3.79-3.95 (m, 4H), 3.67-3.76 (m, 2H), 3.12-3.39 (m, 6H), 2.73-3.07 (m, 8H), 2.04-2.24 (m, 4H), 1.87-2.02 (m, 1H), 1.22-1.75 (m, 12H), 0.96-1.20 (m, 7H), 0.76-0.90 (m, 10H). MS (ESI) m/e 1597.4 (M+H)⁺.

### 1.52. Synthesis of N-[6-(2,5-dioxo-2,5-dihydro-1H-pyrrol-1-yl)hexanoyl]-L-valyl-N-{4-[({[2-({3-[(4-{6-[8-(1,3-benzothiazol-2-ylcarbamoyl)-5-(3-sulfopropoxy)-3,4-dihydroisoquinolin-2(1H)-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](methyl)carbamoyl}oxy)methyl]phenyl}-N⁵-carbamoyl-L-ornithinamide (Synthon JK)

The title compound was prepared by substituting Example 1.52.4 for Example 2.5.3 in Example 2.5.4. ¹H NMR (400 MHz, dimethyl sulfoxide-d₆) δ ppm 9.97 (s, 1H), 7.96-8.11 (m, 2H), 7.67-7.82 (m, 3H), 7.59 (d, 2H), 7.42-7.52 (m, 2H), 7.23-7.36 (m, 4H), 6.91-7.08 (m, 4H), 4.99 (d, 4H), 4.33-4.47 (m, 1H), 4.14-4.23 (m, 4H), 3.86-3.95 (m, 6H), 3.21-3.45 (m, 15H), 2.75-3.07 (m, 9H), 2.56-2.69 (m, 2H), 1.93-2.20 (m, 8H), 0.88-1.72 (m, 20H), 0.74-0.89 (m, 11H). MS (ESI) m/e 1496.3 (M+Na)⁺.

### 1.53. Synthesis of N-[3-(2,5-dioxo-2,5-dihydro-1H-pyrrol-1-yl)propanoyl]-L-valyl-N-{4-[({[2-({3-[(4-{6-[8-(1,3-benzothiazol-2-ylcarbamoyl)-3,4-dihydroisoquinolin-2(1H)-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](2-sulfoethyl)carbamoyl}oxy)methyl]phenyl}-N⁵-carbamoyl-L-ornithinamide (Synthon JJ)

A solution of Example 2.49.1 (0.030 g), 2,5-dioxopyrrolidin-1-yl 3-(2,5-dioxo-2,5-dihydro-1H-pyrrol-1-yl)propanoate (6.34 mg) and N,N-diisopropylethylamine (0.012 mL) in N,N-dimethylformamide (0.5 mL) was stirred at room temperature. After 1 hour the reaction was quenched with a 3:1 mixture of N,N-dimethylformamide:water (1.5 mL). The mixture was purified by reverse phase HPLC using a Gilson system, eluting with 10-85% acetonitrile in water containing 0.1% v/v trifluoroacetic acid. The desired fractions were combined and freeze-dried to provide the title compound. ¹H NMR (500 MHz, dimethyl sulfoxide-d₆) δ ppm 12.85 (s, 1H), 9.99 (s, 1H), 8.18 (q, 1H), 8.12-8.00 (m, 2H), 7.86-7.75 (m, 2H), 7.65-7.55 (m, 3H), 7.53 (dd, 1H), 7.47 (t, 1H), 7.43 (d, 1H), 7.36 (q, 2H), 7.33-7.23 (m, 3H), 6.95 (d, 1H), 6.05 (s, 1H), 5.03-4.92 (m, 4H), 4.39 (q, 1H), 4.24-4.14 (m, 1H), 4.02 (s, 2H), 3.88 (t, 2H), 3.81 (d, 2H), 3.39-3.16 (m, 2H), 3.14-2.86 (m, 10H), 2.68-2.60 (m, 2H), 2.25-2.04 (m, 6H), 2.03-1.90 (m, 1H), 1.78-1.65 (m, 1H), 1.64-1.54 (m, 1H), 1.54-0.90 (m, 20H), 0.89-0.75 (m, 12H). MS (ESI) m/e 1410.1 (M+H)⁺.

### 1.54. Synthesis of N-[(2,5-dioxo-2,5-dihydro-1H-pyrrol-1-yl)acetyl]-L-valyl-N-{4-[({[2-({3-[(4-{6-[8-(1,3-benzothiazol-2-ylcarbamoyl)-3,4-dihydroisoquinolin-2(1H)-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](2-sulfoethyl)carbamoyl}oxy)methyl]phenyl}-N⁵-carbamoyl-L-ornithinamide (Synthon JL)

A solution of Example 2.49.1 (0.039 g), 2,5-dioxopyrrolidin-1-yl 2-(2,5-dioxo-2,5-dihydro-1H-pyrrol-1-yl)acetate (7.81 mg) and N,N-diisopropylethylamine (0.016 mL) in N,N-dimethylformamide (0.5 mL) was stirred at room temperature. After 1 hour, the reaction was quenched with a 3:1 mixture of N,N-dimethylformamide:water (1.5 mL). The mixture was purified by reverse phase HPLC using a Gilson system, eluting with 10-85% acetonitrile in water containing 0.1% v/v trifluoroacetic acid. The desired fractions were combined and freeze-dried to provide the title compound. ¹H NMR (400 MHz, dimethyl sulfoxide-d₆) δ ppm 12.85 (s, 1H), 10.00 (d, 1H), 8.24 (d, 2H), 8.04 (d, 1H), 7.79 (d, 1H), 7.59 (q, 3H), 7.53 (dd, 1H), 7.47 (t, 1H), 7.43 (d, 1H), 7.36 (td, 2H), 7.30 (s, 1H), 7.27 (d, 2H), 7.07 (s, 2H), 6.96 (d, 1H), 5.04-4.85 (m, 4H), 4.39 (q, 2H), 4.26 (dd, 2H), 4.13 (s, 2H), 3.86-3.17 (m, 8H), 3.07-2.81 (m, 4H), 2.63 (t, 2H), 2.09 (s, 3H), 2.03-1.79 (m, 1H), 1.75-1.51 (m, 2H), 1.51-1.03 (m, 12H), 1.01-0.76 (m, 16H). MS (ESI) m/e 1394.4 (M−H)⁻.

### 1.55. Synthesis of 6-[8-(1,3-benzothiazol-2-ylcarbamoyl)-3,4-dihydroisoquinolin-2(1H)-yl]-3-(1-{[3-(2-{[(2S)-2-({[(4-{[(2S,3R,4S,5S,6S)-6-carboxy-3,4,5-trihydroxytetrahydro-2H-pyran-2-yl]oxy}-3-[(3-{[6-(2,5-dioxo-2,5-dihydro-1H-pyrrol-1-yl)hexanoyl]amino}propanoyl)amino]benzyl)oxy]carbonyl}amino)-3-sulfopropanoyl](methyl)amino}ethoxy)-5,7-dimethyltricyclo[3.3.1.1^{3,7}]dec-1-yl]methyl}-5-methyl-1H-pyrazol-4-yl)pyridine-2-carboxylic Acid (Synthon FE)

### 1.55.1. (2S,3R,4S,5S,6S)-2-(4-formyl-2-nitrophenoxy)-6-(methoxycarbonyl)tetrahydro-2H-pyran-3,4,5-triyl Triacetate

To a solution of (2R,3R,4S,5S,6S)-2-bromo-6-(methoxycarbonyl)tetrahydro-2H-pyran-3,4,5-triyl triacetate (4 g) in acetonitrile (100 mL)) was added silver(I) oxide (10.04 g) and 4-hydroxy-3-nitrobenzaldehyde (1.683 g). The reaction mixture was stirred for 4 hours at room temperature and filtered. The filtrate was concentrated, and the residue was purified by silica gel chromatography, eluting with 5-50% ethyl acetate in heptanes, to provide the title compound. MS (ESI) m/e (M+18)⁺.

### 1.55.2. (2S,3R,4S,5S,6S)-2-(4-(hydroxymethyl)-2-nitrophenoxy)-6-(methoxycarbonyl)tetrahydro-2H-pyran-3,4,5-triyl Triacetate

To a solution of Example 2.55.1 (6 g) in a mixture of chloroform (75 mL) and isopropanol (18.75 mL) was added 0.87 g of silica gel. The resulting mixture was cooled to 0° C., NaBH₄ (0.470 g) was added, and the resulting suspension was stirred at 0° C. for 45 minutes. The reaction mixture was diluted with dichloromethane (100 mL) and filtered through diatomaceous earth. The filtrate was washed with water and brine and concentrated to give the crude product, which was used without further purification. MS (ESI) m/e (M+NH₄)⁺:

### 1.55.3. (2S,3R,4S,5S,6S)-2-(2-amino-4-(hydroxymethyl)phenoxy)-6-(methoxycarbonyl)tetrahydro-2H-pyran-3,4,5-triyl Triacetate

A stirred solution of Example 2.55.2 (7 g) in ethyl acetate (81 mL) was hydrogenated at 20° C. under 1 atmosphere H₂, using 10% Pd/C (1.535 g) as a catalyst for 12 hours. The reaction mixture was filtered through diatomaceous earth, and the solvent was evaporated under reduced pressure. The residue was purified by silica gel chromatography, eluting with 95/5 dichloromethane/methanol, to give the title compound.

### 1.55.4. 3-((((9H-fluoren-9-yl)methoxy)carbonyl)amino)propanoic Acid

3-Aminopropanoic acid (4.99 g) was dissolved in 10% aqueous Na₂CO₃ solution (120 mL) in a 500 mL flask and cooled with an ice bath. To the resulting solution, (9H-fluoren-9-yl)methyl carbonochloridate (14.5 g) in 1,4-dioxane (100 mL) was gradually added. The reaction mixture was stirred at room temperature for 4 hours, and water (800 mL) was then added. The aqueous phase layer was separated from the reaction mixture and washed with diethyl ether (3×750 mL). The aqueous layer was acidified with 2N HCl aqueous solution to a pH value of 2 and extracted with ethyl acetate (3×750 mL). The organic layers were combined and concentrated to obtain crude product. The crude product was recrystallized in a mixed solvent of ethyl acetate:hexane 1:2 (300 mL) to give the title compound.

### 1.55.5. (9H-fluoren-9-yl)methyl (3-chloro-3-oxopropyl)carbamate

To a solution of Example 2.55.4 in dichloromethane (160 mL) was added sulfurous dichloride (50 mL). The mixture was stirred at 60° C. for 1 hour. The mixture was cooled and concentrated to give the title compound.

### 1.55.6. (2S,3R,4S,5S,6S)-2-(2-(3-((((9H-fluoren-9-yl)methoxy)carbonyl)amino)propanamido)-4-(hydroxymethyl)phenoxy)-6-(methoxycarbonyl)tetrahydro-2H-pyran-3,4,5-triyl Triacetate

To a solution of Example 2.55.3 (6 g) in dichloromethane (480 mL) was added N,N-diisopropylethylamine (4.60 mL). Example 2.55.5 (5.34 g) was added, and the mixture was stirred at room temperature for 30 minutes. The mixture was poured into saturated aqueous sodium bicarbonate and was extracted with ethyl acetate. The combined extracts were washed with water and brine and were dried over sodium sulfate. Filtration and concentration gave a residue that was purified via radial chromatography, using 0-100% ethyl acetate in petroleum ether as mobile phase, to give the title compound.

### 1.55.7. (2S,3R,4S,5S,6S)-2-(2-(3-((((9H-fluoren-9-yl)methoxy)carbonyl)amino)propanamido)-4-((((4-nitrophenoxy)carbonyl)oxy)methyl)phenoxy)-6-(methoxycarbonyl)tetrahydro-2H-pyran-3,4,5-triyl Triacetate

To a mixture of Example 2.55.6 (5.1 g) in N,N-dimethylformamide (200 mL) was added bis(4-nitrophenyl) carbonate (4.14 g) and N,N-diisopropylethylamine (1.784 mL). The mixture was stirred for 16 hours at room temperature and concentrated under reduced pressure. The crude material was dissolved in dichloromethane and aspirated directly onto a 1 mm radial Chromatotron plate and eluted with 50-100% ethyl acetate in hexanes to give the title compound. MS (ESI) m/e (M+H)⁺.

### 1.55.8. 3-(1-((3-(2-((R)-2-((((3-(3-aminopropanamido)-4-(((2S,3R,4S,5S,6S)-6-carboxy-3,4,5-trihydroxytetrahydro-2H-pyran-2-yl)oxy)benzyl)oxy)carbonyl)amino)-N-methyl-3-sulfopropanamido)ethoxy)-5,7-dimethyladamantan-1-yl)methyl)-5-methyl-1H-pyrazol-4-yl)-6-(8-(benzo[d]thiazol-2-ylcarbamoyl)-3,4-dihydroisoquinolin-2(1H)-yl)picolinic Acid

A solution of Example 1.13.7 (0.055 g) and Example 2.55.7 (0.055 g) were stirred together in N,N-dimethylformamide (1.5 mL) and N,N-diisopropylethylamine (0.053 mL) was added. After stirring for 3 hours, the reaction was diluted with ethyl acetate (75 mL) and washed with water (20 mL) and brine (25 mL), dried over magnesium sulfate, filtered, and concentrated. The residue was dissolved in methanol (1 mL) and treated with lithium hydroxide hydrate (0.025 g) in water (0.6 mL). After stirring for 2 hours, the reaction was quenched with trifluoroacetic acid (0.047 ml) and diluted with N,N-dimethylformamide (1 mL). The mixture was purified by reverse phase HPLC using a Gilson system, eluting with 10-80% acetonitrile in water containing 0.1% v/v trifluoroacetic acid. The desired fractions were combined and freeze-dried to provide the title compound as a trifluoroacetic acid salt.

### 1.55.9. 6-[8-(1,3-benzothiazol-2-ylcarbamoyl)-3,4-dihydroisoquinolin-2(1H)-yl]-3-(1-{[3-(2-{[(2S)-2-({[(4-{[(2S,3R,4S,5S,6S)-6-carboxy-3,4,5-trihydroxytetrahydro-2H-pyran-2-yl]oxy}-3-[(3-([6-(2,5-dioxo-2,5-dihydro-1H-pyrrol-1-yl)hexanoyl]amino}propanoyl)amino]benzyl)oxy]carbonyl}amino)-3-sulfopropanoyl](methyl)amino)ethoxy)-5,7-dimethyltricyclo[3.3.1.1^{3,7}]dec-1-yl]methyl}-5-methyl-1H-pyrazol-4-yl)pyridine-2-carboxylic Acid

A solution of Example 2.55.8 (0.013 g) and 2,5-dioxopyrrolidin-1-yl 6-(2,5-dioxo-2,5-dihydro-1H-pyrrol-1-yl)hexanoate (3.07 mg) were stirred in N,N-dimethylformamide (1 mL) and N,N-diisopropylethylamine (7.90 μl) was added. The reaction was stirred for 1 hour and diluted with N,N-dimethylformamide and water. The mixture was purified by reverse phase HPLC using a Gilson system, eluting with 10-75% acetonitrile in water containing 0.1% v/v trifluoroacetic acid. The desired fractions were combined and freeze-dried to provide the title compound. ¹H NMR (400 MHz, dimethyl sulfoxide-d₆) δ ppm 12.84 (s, 1H), 9.07 (s, 1H), 8.15 (s, 1H), 8.04 (d, 1H), 7.89 (t, 1H), 7.79 (d, 1H), 7.61 (d, 1H), 7.56-7.50 (m, 1H), 7.47 (t, 1H), 7.43 (d, 1H), 7.39-7.32 (m, 2H), 7.31 (s, 1H), 7.28 (d, 1H), 7.06 (d, 1H), 7.04-6.92 (m, 4H), 5.00-4.79 (m, 5H), 4.73-4.64 (m, 1H), 3.94-3.78 (m, 4H), 3.57-2.84 (m, 12H), 2.84-2.56 (m, 6H), 2.14-1.73 (m, 5H), 1.57-0.89 (m, 22H), 0.84 (s, 6H). MS (ESI) m/e 1516.2 (M−H)⁻.

### 1.56. Synthesis of 4-[(1E)-3-({[2-({3-[(4-{2-carboxy-6-[8-([1,3]thiazolo[5,4-b]pyridin-2-ylcarbamoyl)-3,4-dihydroisoquinolin-2(1H)-yl]pyridin-3-yl}-5-methyl-1H-pyrazol-1-yl)methyl]-5,7-dimethyltricyclo[3.3.1.1^{3,7}]dec-1-yl}oxy)ethyl](2-sulfoethyl)carbamoyl}oxy)prop-1-en-1-yl]-2-({N-[6-(2,5-dioxo-2,5-dihydro-1H-pyrrol-1-yl)hexanoyl]-beta-alanyl}amino)phenyl Beta-D-Glucopyranosiduronic Acid (Synthon GG)

### 1.56.1. 3-(1-((3-(2-(((((E)-3-(3-(3-aminopropanamido)-4-(((2S,3R,4S,5S,6S)-6-carboxy-3,4,5-trihydroxytetrahydro-2H-pyran-2-yl)oxy)phenyl)allyl)oxy)carbonyl)(2-sulfoethyl)amino)ethoxy)-5,7-dimethyladamantan-1-yl)methyl)-5-methyl-1H-pyrazol-4-yl)-6-(8-(thiazolo[5,4-b]pyridin-2-ylcarbamoyl)-3,4-dihydroisoquinolin-2(1H)-yl)picolinic Acid

Example 1.22.5 (48 mg) was dissolved in dimethylformamide (0.5 mL), and Example 2.44.7 (55 mg) and N,N-diisopropylethylamine (90 μL) were added. The reaction mixture was stirred at room temperature overnight. The reaction was concentrated, and the residue was dissolved in methanol (1 mL) and 1.94N aqueous LiOH (0.27 mL) was added. The mixture was stirred at room temperature for one hour. Purification of the mixture by reverse phase chromatography (C18 column), eluting with 10-90% acetonitrile in water containing 0.1% v/v trifluoroacetic acid, provided the title compound as a trifluoroacetic acid salt. MS (ESI−) m/e 1291.4 (M−H)⁻.

### 1.56.2. 4-[(1E)-3-({[2-({3-[(4-{2-carboxy-6-[8-([1,3]thiazolo[5,4-b]pyridin-2-ylcarbamoyl)-3,4-dihydroisoquinolin-2(1H)-yl]pyridin-3-yl}-5-methyl-1H-pyrazol-1-yl)methyl]-5,7-dimethyltricyclo[3.3.1.1^{3,7}]dec-1-yl}oxy)ethyl](2-sulfoethyl)carbamoyl}oxy)prop-1-en-1-yl]-2-({N-[6-(2,5-dioxo-2,5-dihydro-1H-pyrrol-1-yl)hexanoyl]-beta-alanyl}amino)phenyl Beta-D-Glucopyranosiduronic Acid

The title compound was prepared by substituting Example 1.56.1 for Example 1.2.9 in Example 2.1. ¹H NMR (400 MHz, dimethyl sulfoxide-d₆) δ ppm 13.00 (v br s, 1H), 9.03 (s, 1H), 8.53 (dd, 1H), 8.24 (s, 1H), 8.16 (dd, 1H), 7.90 (br s, 1H), 7.61 (d, 1H), 7.54 (d, 1H) 7.52 (d, 1H), 7.44 (d, 1H), 7.37 (t, 1H), 7.30 (s, 1H), 7.11 (br d, 1H), 7.03 (d, 1H), 6.98 (s, 2H), 6.97 (d, 1H), 6.58 (m, 1H), 6.15 (m, 1H), 4.96 (s, 2H), 4.88 (br m, 1H), 4.64 (br m, 2H), 3.88 (m, 3H), 3.79 (br m, 2H), 3.27-3.48 (m, 14H), 3.01 (m, 2H), 2.67 (br m, 2H), 2.54 (m, 2H), 2.09 (s, 3H), 2.03 (t, 2H), 1.45 (m, 6H), 1.37 (br m, 2H), 1.28-0.90 (m, 10H), 0.77-0.82 (m, 6H). MS (ESI) m/e 1484.4 (M−H)⁻.

### 1.57. Synthesis of 4-[(1E)-3-({[2-({3-[(4-{2-carboxy-6-[8-([1,3]thiazolo[4,5-b]pyridin-2-ylcarbamoyl)-3,4-dihydroisoquinolin-2(1H)-yl]pyridin-3-yl}-5-methyl-1H-pyrazol-1-yl)methyl]-5,7-dimethyltricyclo[3.3.1.1^{3,7}]dec-1-yl}oxy)ethyl](2-sulfoethyl)carbamoyl}oxy)prop-1-en-1-yl]-2-({N-[6-(2,5-dioxo-2,5-dihydro-1H-pyrrol-1-yl)hexanoyl]-beta-alanyl}amino)phenyl Beta-D-Glucopyranosiduronic Acid (Synthon GM)

### 1.57.1. 3-(1-((3-(2-(((((E)-3-(3-(3-aminopropanamido)-4-(((2S,3R,4S,5S,6S)-6-carboxy-3,4,5-trihydroxytetrahydro-2H-pyran-2-yl)oxy)phenyl)allyl)oxy)carbonyl)(2-sulfoethyl)amino)ethoxy)-5,7-dimethyladamantan-1-yl)methyl)-5-methyl-1H-pyrazol-4-yl)-6-(8-(thiazolo[4,5-b]pyridin-2-ylcarbamoyl)-3,4-dihydroisoquinolin-2(1H)-yl)picolinic Acid

The title compound was prepared by substituting Example 1.23.4 for Example 1.22.5 in Example 2.56.1. MS (ESI) m/e 1291.4 (M−H)⁻.

### 1.57.2. 4-[(1E)-3-({[2-({3-[(4-{2-carboxy-6-[8-([1,3]thiazolo[4,5-b]pyridin-2-ylcarbamoyl)-3,4-dihydroisoquinolin-2(1H)-yl]pyridin-3-yl}-5-methyl-1H-pyrazol-1-yl)methyl]-5,7-dimethyltricyclo[3.3.1.1^{3,7}]dec-1-yl}oxy)ethyl](2-sulfoethyl)carbamoyl}oxy)prop-1-en-1-yl]-2-({N-[6-(2,5-dioxo-2,5-dihydro-1H-pyrrol-1-yl)hexanoyl]-beta-alanyl}amino)phenyl Beta-D-Glucopyranosiduronic Acid

The title compound was prepared by substituting Example 1.57.1 for Example 1.2.9 in Example 2.1. ¹H NMR (500 MHz, dimethyl sulfoxide-d₆) δ ppm 9.03 (s, 1H), 8.72 (d, 1H), 8.66 (d, 1H), 8.25 (s, 1H), 7.89 (br m, 1H), 7.65 (d, 1H), 7.52 (br m, 2H), 7.46 (d, 1H), 7.39 (t, 1H), 7.30 (s, 1H), 7.11 (br d, 1H), 7.03 (d, 1H), 6.98 (s, 2H), 6.97 (d, 1H), 6.58 (m, 1H), 6.15 (m, 1H), 4.96 (s, 2H), 4.88 (br m, 1H), 4.64 (br m, 2H), 3.88 (m, 3H), 3.79 (br m, 2H), 3.27-3.48 (m, 14H), 3.01 (m, 2H), 2.67 (br m, 2H), 2.54 (m, 2H), 2.09 (s, 3H), 2.03 (t, 2H), 1.45 (m, 6H), 1.37 (br m, 2H), 1.28-0.90 (m, 10H), 0.77-0.82 (m, 6H). MS (ESI) m/e 1484.4 (M−H)⁻.

### 1.58. Synthesis of 4-[(1E)-3-({[2-({3-[(4-{6-[8-(1,3-benzothiazol-2-ylcarbamoyl)-3,4-dihydroisoquinolin-2(1H)-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](2-sulfoethyl)carbamoyl}oxy)prop-1-en-1-yl]-2-({N-[6-(2,5-dioxo-2,5-dihydro-1H-pyrrol-1-yl)hexanoyl]-beta-alanyl}amino)phenyl Beta-D-Glucopyranosiduronic Acid (Synthon HD)

### 1.58.1. 3-(1-((3-(2-(((((E)-3-(3-(3-aminopropanamido)-4-(((2S,3R,4S,5S,6S)-6-carboxy-3,4,5-trihydroxytetrahydro-2H-pyran-2-yl)oxy)phenyl)allyl)oxy)carbonyl)(2-sulfoethyl)amino)ethoxy)-5,7-dimethyladamantan-1-yl)methyl)-5-methyl-1H-pyrazol-4-yl)-6-(8-(benzo[d]thiazol-2-ylcarbamoyl)-3,4-dihydroisoquinolin-2(1H)-yl)picolinic Acid

The title compound was prepared by substituting Example 1.2.9 for Example 1.22.5 in Example 2.56.1. MS (ESI−) m/e 1290.2 (M−H)⁻.

### 1.58.2. 4-[(1E)-3-({[2-({3-[(4-{6-[8-(1,3-benzothiazol-2-ylcarbamoyl)-3,4-dihydroisoquinolin-2(1H)-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](2-sulfoethyl)carbamoyl}oxy)prop-1-en-1-yl]-2-({N-[6-(2,5-dioxo-2,5-dihydro-1H-pyrrol-1-yl)hexanoyl]-beta-alanyl}amino)phenyl Beta-D-Glucopyranosiduronic Acid

The title compound was prepared by substituting Example 1.58.1 for Example 1.56.1 in Example 2.56.2. ¹H NMR (400 MHz, dimethyl sulfoxide-d₆) δ ppm 9.03 (s, 1H), 8.25 (s, 1H), 8.03 (d, 1H), 7.89 (br m, 1H), 7.79 (d, 1H), 7.61 (d, 1H), 7.53 (br m, 1H), 7.46 (m, 2H), 7.37 (m, 2H), 7.32 (s, 1H), 7.11 (br d, 1H), 7.03 (d, 1H), 6.98 (s, 2H), 6.97 (d, 1H), 6.58 (m, 1H), 6.15 (m, 1H), 4.96 (s, 2H), 4.88 (br m, 1H), 4.64 (br m, 2H), 3.88 (m, 3H), 3.79 (br m, 2H), 3.27-3.48 (m, 14H), 3.01 (m, 2H), 2.67 (br m, 2H), 2.54 (m, 2H), 2.09 (s, 3H), 2.03 (t, 2H), 1.45 (m, 6H), 1.37 (br m, 2H), 1.28-0.90 (m, 10H), 0.77-0.82 (m, 6H). MS (ESI−) m/e 1483.3 (M−H)⁻.

### 1.59. Synthesis of 4-[(1E)-3-({[2-({3-[(4-{2-carboxy-6-[8-([1,3]thiazolo[5,4-b]pyridin-2-ylcarbamoyl)-3,4-dihydroisoquinolin-2(1H)-yl]pyridin-3-yl}-5-methyl-1H-pyrazol-1-yl)methyl]-5,7-dimethyltricyclo[3.3.1.1^{3,7}]dec-1-yl}oxy)ethyl](3-phosphonopropyl) carbamoyl}oxy)prop-1-en-1-yl]-2-({N-[6-(2,5-dioxo-2,5-dihydro-1H-pyrrol-1-yl)hexanoyl]-beta-alanyl}amino)phenyl Beta-D-Glucopyranosiduronic Acid (Synthon HS)

### 1.59.1. 3-(1-((3-(2-(((((E)-3-(3-(3-aminopropanamido)-4-(((2S,3R,4S,5S,6S)-6-carboxy-3,4,5-trihydroxytetrahydro-2H-pyran-2-yl)oxy)phenyl)allyl)oxy)carbonyl)(3-phosphonopropyl)amino)ethoxy)-5,7-dimethyladamantan-1-yl)methyl)-5-methyl-1H-pyrazol-4-yl)-6-(8-(thiazolo[5,4-b]pyridin-2-ylcarbamoyl)-3,4-dihydroisoquinolin-2(1H)-yl)picolinic Acid

The title compound was prepared by substituting Example 1.40.2 for Example 1.22.5 in Example 2.56.1. MS (ESI−) m/e 1305.4 (M−H)⁻.

### 1.59.2. 4-[(1E)-3-({[2-({3-[(4-{2-carboxy-6-[8-([1,3]thiazolo[5,4-b]pyridin-2-ylcarbamoyl)-3,4-dihydroisoquinolin-2(1H)-yl]pyridin-3-yl}-5-methyl-1H-pyrazol-1-yl)methyl]-5,7-dimethyltricyclo[3.3.1.1^{3,7}]dec-1-yl}oxy)ethyl](3-phosphonopropyl) carbamoyl}oxy)prop-1-en-1-yl]-2-({N-[6-(2,5-dioxo-2,5-dihydro-1H-pyrrol-1-yl)hexanoyl]-beta-alanyl}amino)phenyl Beta-D-Glucopyranosiduronic Acid

The title compound was prepared by substituting Example 1.59.1 for Example 1.56.1 in Example 2.56.2. ¹H NMR (400 MHz, dimethyl sulfoxide-d₆) δ ppm 9.03 (s, 1H), 8.53 (dd, 1H), 8.24 (s, 1H), 8.16 (dd, 1H), 7.90 (br s, 1H), 7.61 (d, 1H), 7.54 (d, 1H) 7.52 (d, 1H), 7.44 (d, 1H), 7.37 (t, 1H), 7.28 (s, 1H), 7.11 (br d, 1H), 7.03 (d, 1H), 6.98 (s, 2H), 6.97 (d, 1H), 6.56 (m, 1H), 6.16 (m, 1H), 4.96 (s, 2H), 4.86 (br m, 1H), 4.64 (br d, 2H), 3.88 (m, 3H), 3.79 (br m, 2H), 3.27-3.44 (m, 14H), 3.01 (m, 2H), 2.54 (m, 2H), 2.08 (s, 3H), 2.03 (t, 2H), 1.46 (m, 6H), 1.37 (br m, 2H), 1.28-0.90 (m, 10H), 0.77-0.82 (m, 6H). MS (ESI) m/e 1498.4 (M−H)⁻.

### 1.60. Synthesis of 4-[(1E)-3-({[2-({3-[(4-{6-[8-(1,3-benzothiazol-2-ylcarbamoyl)-5-(3-phosphonopropoxy)-3,4-dihydroisoquinolin-2(1H)-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](methyl)carbamoyl}oxy)prop-1-en-1-yl]-2-({N-[6-(2,5-dioxo-2,5-dihydro-1H-pyrrol-1-yl)hexanoyl]-beta-alanyl}amino)phenyl Beta-D-Glucopyranosiduronic Acid (Synthon HW)

### 1.60.1. 3-(1-(((3-(2-(((((E)-3-(3-(3-aminopropanamido)-4-(((2S,3R,4S,5S,6S)-6-carboxy-3,4,5-trihydroxytetrahydro-2H-pyran-2-yl)oxy)phenyl)allyl)oxy)carbonyl)(methyl)amino)ethoxy)-5,7-dimethyladamantan-1-yl)methyl)-5-methyl-1H-pyrazol-4-yl)-6-(8-(benzo[d]thiazol-2-ylcarbamoyl)-5-(3-phosphonopropoxy)-3,4-dihydroisoquinolin-2(1H)-yl)picolinic Acid

The title compound was prepared by substituting Example 1.31.11 for Example 1.22.5 in Example 2.56.1. MS (ESI) m/e 1336.2 (M+Na)+.

### 1.60.2. 4-[(1E)-3-({[2-({3-[(4-{6-[8-(1,3-benzothiazol-2-ylcarbamoyl)-5-(3-phosphonopropoxy)-3,4-dihydroisoquinolin-2(1H)-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](methyl)carbamoyl}oxy)prop-1-en-1-yl]-2-({N-[6-(2,5-dioxo-2,5-dihydro-1H-pyrrol-1-yl)hexanoyl]-beta-alanyl}amino)phenyl Beta-D-Glucopyranosiduronic Acid

The title compound was prepared by substituting Example 1.60.1 for Example 1.56.1 in Example 2.56.2. ¹H NMR (400 MHz, dimethyl sulfoxide-d₆) δ ppm 9.03 (s, 1H) 8.25 (s, 1H), 8.01 (d, 1H), 7.83-7.91 (m, 1H), 7.75 (dd, 2H), 7.42-7.58 (m, 2H), 7.34 (t, 1H), 7.28 (s, 1H), 6.93-7.15 (m, 6H), 6.56 (d, 1H), 6.09-6.24 (m, 1H), 5.01 (s, 3H), 4.80-4.92 (m, 2H), 4.57-4.69 (m, 3H), 4.12-4.21 (m, 6H), 3.86-3.94 (m, 7H), 3.28-3.47 (m, 12H), 2.77-2.96 (m, 6H), 2.52-2.58 (m, 2H), 2.09 (s, 3H), 1.90-2.05 (m, 4H), 1.65-1.78 (m, 2H), 0.90-1.53 (m, 16H), 0.80 (m, 6H). MS (ESI) m/e 1529.5 (M+H)⁺.

### 1.61. Synthesis of 4-[(1E)-3-({[2-({3-[(4-{6-[8-(1,3-benzothiazol-2-ylcarbamoyl)-3,4-dihydroisoquinolin-2(1H)-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](3-phosphonopropyl) carbamoyl}oxy)prop-1-en-1-yl]-2-({N-[6-(2,5-dioxo-2,5-dihydro-1H-pyrrol-1-yl)hexanoyl]-beta-alanyl}amino)phenyl Beta-D-Glucopyranosiduronic Acid (Synthon HX)

### 1.61.1. 3-(1-((3-(2-(((((E)-3-(3-(3-aminopropanamido)-4-(((2S,3R,4S,5S,6S)-6-carboxy-3,4,5-trihydroxytetrahydro-2H-pyran-2-yl)oxy)phenyl)allyl)oxy)carbonyl)(3-phosphonopropyl)amino)ethoxy)-5,7-dimethyladamantan-1-yl)methyl)-5-methyl-1H-pyrazol-4-yl)-6-(8-(benzo[d]thiazol-2-ylcarbamoyl)-3,4-dihydroisoquinolin-2(1H)-yl)picolinic Acid

The title compound was prepared by substituting Example 1.14.4 for Example 1.22.5 in Example 2.56.1. MS (ESI) m/e 1304.3 (M−H)⁻.

### 1.61.2. 4-[(1E)-3-({[2-({3-[(4-{6-[8-(1,3-benzothiazol-2-ylcarbamoyl)-3,4-dihydroisoquinolin-2(1H)-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](3-phosphonopropyl) carbamoyl}oxy)prop-1-en-1-yl]-2-({N-[6-(2,5-dioxo-2,5-dihydro-1H-pyrrol-1-yl)hexanoyl]-beta-alanyl}amino)phenyl Beta-D-Glucopyranosiduronic Acid

The title compound was prepared by substituting Example 1.61.1 for Example 1.56.1 in Example 2.56.2. ¹H NMR (400 MHz, dimethyl sulfoxide-d₆) δ ppm 9.03 (s, 1H), 8.25 (br s, 1H), 8.03 (d, 1H), 7.89 (br m, 1H), 7.79 (d, 1H), 7.61 (d, 1H), 7.53 (br m, 1H), 7.46 (m, 2H), 7.37 (m, 2H), 7.28 (s, 1H), 7.11 (br d, 1H), 7.03 (d, 1H), 6.98 (s, 2H), 6.97 (d, 1H), 6.56 (m, 1H), 6.17 (m, 1H), 4.96 (s, 2H), 4.86 (br m, 1H), 4.64 (br d, 2H), 3.88 (m, 3H), 3.79 (br m, 2H), 3.27-3.44 (m, 14H), 3.01 (m, 2H), 2.54 (m, 2H), 2.08 (s, 3H), 2.03 (t, 2H), 1.46 (m, 6H), 1.37 (br m, 2H), 1.28-0.90 (m, 10H), 0.77-0.82 (m, 6H). MS (ESI−) m/e 1497.4 (M−H)⁻.

### 1.62. Synthesis of 4-[({[2-({3-[(4-{6-[8-(1,3-benzothiazol-2-ylcarbamoyl)-3,4-dihydroisoquinolin-2(1H)-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](2-sulfoethyl)carbamoyl}oxy)methyl]-3-[2-(2-{[3-(2,5-dioxo-2,5-dihydro-1H-pyrrol-1-yl)propanoyl]amino}ethoxy)ethoxy]phenyl Beta-D-Glucopyranosiduronic Acid (Synthon HY)

### 1.62.1. (2S,3R,4S,5S,6S)-2-(4-formyl-3-hydroxyphenoxy)-6-(methoxycarbonyl)tetrahydro-2H-pyran-3,4,5-triyl Triacetate

2,4-Dihydroxybenzaldehyde (15 g) and (2S,3R,4S,5S,6S)-2-bromo-6-(methoxycarbonyl)tetrahydro-2H-pyran-3,4,5-triyl triacetate (10 g) were dissolved in acetonitrile followed by the addition of silver carbonate (10 g) and the reaction was heated to 49° C. After stirring for 4 hours, the reaction was cooled, filtered and concentrated. The crude title compound was suspended in dichloromethane and was filtered through diatomaceous earth and concentrated. The residue was purified by silica gel chromatography eluting with 1-100% ethyl acetate/heptane to provide the title compound.

### 1.62.2. (2S,3R,4S,5S,6S)-2-(3-hydroxy-4-(hydroxymethyl)phenoxy)-6-(methoxycarbonyl)tetrahydro-2H-pyran-3,4,5-triyl Triacetate

A solution of Example 2.62.1 (16.12 g) in tetrahydrofuran (200 mL) and methanol (200 mL) was cooled to 0° C. and sodium borohydride (1.476 g) was added portionwise. The reaction was stirred for 20 minutes and was quenched with a 1:1 mixture of water:aqueous saturated sodium bicarbonate solution (400 mL). The resulting solids were filtered off and rinsed with ethyl acetate. The phases were separated and the aqueous layer was extracted four times with ethyl acetate. The combined organic layers were dried over magnesium sulfate, filtered, and concentrated. The crude title compound was purified via silica gel chromatography eluting with 1-100% ethyl acetate/heptanes to provide the title compound. MS (ESI) m/e 473.9 (M+NH₄)⁺.

### 1.62.3. (2S,3R,4S,5S,6S)-2-(4-(((tert-butyldimethylsilyl)oxy)methyl)-3-hydroxyphenoxy)-6-(methoxycarbonyl)tetrahydro-2H-pyran-3,4,5-triyl Triacetate

Example 2.62.2 (7.66 g) and tert-butyldimethylsilyl chloride (2.78 g) in dichloromethane (168 mL) at −5° C. was added imidazole (2.63 g) and the reaction was stirred overnight allowing the internal temperature of the reaction to warm to 12° C. The reaction mixture was poured into saturated aqueous ammonium chloride and extracted four times with dichloromethane. The combined organics were washed with brine, dried over magnesium sulfate, filtered and concentrated. The crude title compound was purified via silica gel chromatography eluting with 1-50% ethyl acetate/heptanes to provide the title compound. MS (ESI) m/e 593.0 (M+Na)+.

### 1.62.4. (2S,3R,4S,5S,6S)-2-(3-(2-(2-((((9H-fluoren-9-yl)methoxy)carbonyl)amino)ethoxy)ethoxy)-4-(((tert-butyldimethylsilyl)oxy)methyl)phenoxy)-6-(methoxycarbonyl)tetrahydro-2H-pyran-3,4,5-triyl Triacetate

To Example 2.62.3 (5.03 g) and triphenylphosphine (4.62 g) in toluene (88 mL) was added di-tert-butyl-azodicarboxylate (4.06 g) and the reaction was stirred for 30 minutes. (9H-Fluoren-9-yl)methyl (2-(2-hydroxyethoxy)ethyl)carbamate was added and the reaction was stirred for an addition 1.5 hours. The reaction was loaded directly onto silica gel and was eluted with 1-50% ethyl acetate/heptanes to provide the title compound.

### 1.62.5. (2S,3R,4S,5S,6S)-2-(3-(2-(2-((((9H-fluoren-9-yl)methoxy)carbonyl)amino)ethoxy)ethoxy)-4-(hydroxymethyl)phenoxy)-6-(methoxycarbonyl)tetrahydro-2H-pyran-3,4,5-triyl Triacetate

Example 2.62.4 (4.29 g) was stirred in a 3:1:1 solution of acetic acid:water:tetrahydrofuran (100 mL) overnight. The reaction was poured into saturated aqueous sodium bicarbonate and extracted with ethyl acetate. The organic layer was dried over magnesium sulfate, filtered and concentrated. The crude title compound was purified via silica gel chromatography, eluting with 1-50% ethyl acetate/heptanes to provide the title compound.

### 1.62.6. (2S,3R,4S,5S,6S)-2-(3-(2-(2-((((9H-fluoren-9-yl)methoxy)carbonyl)amino)ethoxy)ethoxy)-4-((((4-nitrophenoxy)carbonyl)oxy)methyl)phenoxy)-6-(methoxycarbonyl)tetrahydro-2H-pyran-3,4,5-triyl Triacetate

To a solution of Example 2.62.5 (0.595 g) and bis(4-nitrophenyl) carbonate (0.492 g) in N,N-dimethylformamide (4 mL) was added N-ethyl-N-isopropylpropan-2-amine (0.212 mL). After 1.5 hours, the reaction was concentrated under high vacuum. The reaction was loaded directly onto silica gel and eluted using 1-50% ethyl acetate/heptanes to provide the title compound. MS (ESI) m/e 922.9 (M+Na)⁺.

### 1.62.7. 3-(1-((3-(2-((((2-(2-(2-aminoethoxy)ethoxy)-4-(((2S,3R,4S,5S,6S)-6-carboxy-3,4,5-trihydroxytetrahydro-2H-pyran-2-yl)oxy)benzyl)oxy)carbonyl)(2-sulfoethyl)amino)ethoxy)-5,7-dimethyladamantan-1-yl)methyl)-5-methyl-1H-pyrazol-4-yl)-6-(8-(benzo[d]thiazol-2-ylcarbamoyl)-3,4-dihydroisoquinolin-2(1H)-yl)picolinic Acid

To a solution of Example 1.2.9 (0.073 g) and Example 2.62.6 (0.077 g) in N,N-dimethylformamide (0.5 mL) was added N,N-diisopropylethylamine (0.066 mL), and the reaction was stirred overnight. The reaction was concentrated, and the residue was dissolved in tetrahydrofuran (0.5 mL) and methanol (0.5 mL) and treated with lithium hydroxide monohydrate (0.047 g) as a solution in water (0.5 mL). After 1 hour, the reaction was diluted with N,N-dimethylformamide and water and was quenched by the addition of trifluoroacetic acid (0.116 mL). The mixture was purified by reverse phase HPLC using a Gilson system, eluting with 10-75% acetonitrile in water containing 0.1% v/v trifluoroacetic acid. The desired fractions were combined and freeze-dried to provide the title compound.

### 1.62.8. 4-[({[2-({3-[(4-{6-[8-(1,3-benzothiazol-2-ylcarbamoyl)-3,4-dihydroisoquinolin-2(1H)-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](2-sulfoethyl)carbamoyl}oxy)methyl]-3-[2-(2-{[3-(2,5-dioxo-2,5-dihydro-1H-pyrrol-1-yl)propanoyl]amino}ethoxy)ethoxy]phenyl Beta-D-Glucopyranosiduronic Acid

A solution of Example 2.62.7 (0.053 g), 2,5-dioxopyrrolidin-1-yl 3-(2,5-dioxo-2,5-dihydro-1H-pyrrol-1-yl)propanoate (0.012 g) and N,N-diisopropylethylamine (0.033 mL) in N,N-dimethylformamide (0.75 mL) was stirred at room temperature. After stirring for 1 hour, the reaction was diluted with N,N-dimethylformamide and water. The mixture was purified by reverse phase HPLC using a Gilson system, eluting with 10-75% acetonitrile in water containing 0.1% v/v trifluoroacetic acid. The desired fractions were combined and freeze-dried to provide the title compound. ¹H NMR (400 MHz, dimethyl sulfoxide-d₆) δ ppm 12.85 (s, 1H), 8.04 (d, 2H), 7.79 (d, 1H), 7.61 (d, 1H), 7.54 (d, 1H), 7.51-7.40 (m, 2H), 7.40-7.31 (m, 3H), 7.20 (d, 1H), 7.00-6.94 (m, 3H), 6.73-6.57 (m, 2H), 5.06 (t, 1H), 5.01-4.91 (m, 4H), 3.96-3.85 (m, 2H), 3.85-3.78 (m, 2H), 3.78-3.69 (m, 2H), 3.59 (t, 2H), 3.53-3.34 (m, 6H), 3.34-3.21 (m, 4H), 3.17 (q, 2H), 3.02 (t, 2H), 2.66 (t, 2H), 2.33 (t, 2H), 2.10 (s, 3H), 1.44-0.90 (m, 16H), 0.83 (d, 6H). MS (−ESI) m/e 1432.4 (M−H)⁻.

### 1.63. Synthesis of 4-[(1E)-3-({[2-({3-[(4-{2-carboxy-6-[8-([1,3]thiazolo[4,5-b]pyridin-2-ylcarbamoyl)-3,4-dihydroisoquinolin-2(1H)-yl]pyridin-3-yl}-5-methyl-1H-pyrazol-1-yl)methyl]-5,7-dimethyltricyclo[3.3.1.1^{3,7}]dec-1-yl}oxy)ethyl](3-phosphonopropyl) carbamoyl}oxy)prop-1-en-1-yl]-2-({N-[6-(2,5-dioxo-2,5-dihydro-1H-pyrrol-1-yl)hexanoyl]-beta-alanyl}amino)phenyl Beta-D-Glucopyranosiduronic Acid (Synthon IB)

### 1.63.1. 3-(1-((3-(2-(((((E)-3-(3-(3-aminopropanamido)-4-(((2S,3R,4S,5S,6S)-6-carboxy-3,4,5-trihydroxytetrahydro-2H-pyran-2-yl)oxy)phenyl)allyl)oxy)carbonyl)(3-phosphonopropyl)amino)ethoxy)-5,7-dimethyladamantan-1-yl)methyl)-5-methyl-1H-pyrazol-4-yl)-6-(8-(thiazolo[4,5-b]pyridin-2-ylcarbamoyl)-3,4-dihydroisoquinolin-2(1H)-yl)picolinic Acid

The title compound was prepared by substituting Example 1.39.2 for Example 1.22.5 in Example 2.56.1.

### 1.63.2. 4-[(1E)-3-({[2-({3-[(4-{2-carboxy-6-[8-([1,3]thiazolo[4,5-b]pyridin-2-ylcarbamoyl)-3,4-dihydroisoquinolin-2(1H)-yl]pyridin-3-yl}-5-methyl-1H-pyrazol-1-yl)methyl]-5,7-dimethyltricyclo[3.3.1.1^{3,7}]dec-1-yl}oxy)ethyl](3-phosphonopropyl) carbamoyl}oxy)prop-1-en-1-yl]-2-({N-[6-(2,5-dioxo-2,5-dihydro-1H-pyrrol-1-yl)hexanoyl]-beta-alanyl}amino)phenyl Beta-D-Glucopyranosiduronic Acid

The title compound was prepared by substituting Example 2.63.1 for Example 1.56.1 in Example 2.56.2. ¹H NMR (400 MHz, dimethyl sulfoxide-d₆) δ ppm 9.03 (s, 1H), 8.61 (d, 1H), 8.55 (d, 1H), 8.25 (br s, 1H), 7.89 (br m, 1H), 7.65 (d, 1H), 7.50 (br d, 1H), 7.46 (d, 1H), 7.39 (m, 2H), 7.28 (s, 1H), 7.11 (br d, 1H), 7.03 (d, 1H), 6.98 (s, 2H), 6.97 (d, 1H), 6.56 (m, 1H), 6.17 (m, 1H), 4.97 (s, 2H), 4.86 (br m, 1H), 4.64 (br d, 2H), 3.88 (m, 3H), 3.79 (br m, 2H), 3.27-3.44 (m, 14H), 3.01 (m, 2H), 2.54 (m, 2H), 2.08 (s, 3H), 2.03 (t, 2H), 1.46 (m, 6H), 1.37 (br m, 2H), 1.28-0.90 (m, 10H), 0.77-0.82 (m, 6H). MS (ESI) m/e 1498.3 (M−H)⁻.

### 1.64. Synthesis of 6-[8-(1,3-benzothiazol-2-ylcarbamoyl)-3,4-dihydroisoquinolin-2(1H)-yl]-3-{1-[(3-{2-[(2-carboxyethyl) ({[(2E)-3-(4-{[(2S,3R,4S,5S,6S)-6-carboxy-3,4,5-trihydroxytetrahydro-2H-pyran-2-yl]oxy}-3-[(3-{[6-(2,5-dioxo-2,5-dihydro-1H-pyrrol-1-yl)hexanoyl]amino}propanoyl)amino]phenyl)prop-2-en-1-yl]oxy}carbonyl)amino]ethoxy}-5,7-dimethyltricyclo[3.3.1.1^{3,7}]dec-1-yl)methyl]-5-methyl-1H-pyrazol-4-yl}pyridine-2-carboxylic Acid (Synthon IE)

### 1.64.1. 3-(1-((3-(2-(((((E)-3-(3-(3-aminopropanamido)-4-(((2S,3R,4S,5S,6S)-6-carboxy-3,4,5-trihydroxytetrahydro-2H-pyran-2-yl)oxy)phenyl)allyl)oxy)carbonyl)(2-carboxyethyl)amino)ethoxy)-5,7-dimethyladamantan-1-yl)methyl)-5-methyl-1H-pyrazol-4-yl)-6-(8-(benzo[d]thiazol-2-ylcarbamoyl)-3,4-dihydroisoquinolin-2(1H)-yl)picolinic Acid, Trifluoroacetic Acid Salt

To a solution of Example 1.25.2 (0.050 g) and Example 2.44.7 (0.061 g) in N,N-dimethylformamide (1 mL) was added N,N-diisopropylethylamine (0.047 mL), and the reaction was stirred at room temperature overnight. The reaction was concentrated, and the residue was dissolved in methanol (0.5 mL) and tetrahydrofuran (0.5 mL) and treated with a solution of lithium hydroxide hydrate (0.034 g) in water (0.5 mL). The reaction was stirred at room temperature for 1 hour. The reaction was quenched with trifluoroacetic acid (0.083 mL) and diluted with N,N-dimethylformamide (1 mL). The mixture was purified by reverse phase HPLC using a Gilson system, eluting with 10-75% acetonitrile in water containing 0.1% v/v trifluoroacetic acid. The desired fractions were combined and freeze-dried to provide the title compound

### 1.64.2. 6-[8-(1,3-benzothiazol-2-ylcarbamoyl)-3,4-dihydroisoquinolin-2(1H)-yl]-3-{1-[(3-{2-[(2-carboxyethyl)({[(2E)-3-(4-{[(2S,3R,4S,5S,6S)-6-carboxy-3,4,5-trihydroxytetrahydro-2H-pyran-2-yl]oxy}-3-[(3-{[6-(2,5-dioxo-2,5-dihydro-1H-pyrrol-1-yl)hexanoyl]amino}propanoyl)amino]phenyl)prop-2-en-1-yl]oxy}carbonyl)amino]ethoxy}-5,7-dimethyltricyclo[3.3.1.1^{3,7}]dec-1-yl)methyl]-5-methyl-1H-pyrazol-4-yl}pyridine-2-carboxylic Acid

To a solution of Example 2.64.1 (0.042 g) and 2,5-dioxopyrrolidin-1-yl 6-(2,5-dioxo-2,5-dihydro-1H-pyrrol-1-yl)hexanoate (10 mg) in N,N-dimethylformamide (0.5 mL) was added N,N-diisopropylethylamine (0.027 mL), and the reaction was stirred at room temperature for 2 hours. The reaction was diluted with N,N-dimethylformamide (1 mL) and water (0.5 mL). The mixture was purified by reverse phase HPLC using a Gilson system, eluting with 10-75% acetonitrile in water containing 0.1% v/v trifluoroacetic acid. The desired fractions were combined and freeze-dried to provide the title compound. ¹H NMR (400 MHz, dimethyl sulfoxide-d₆) δ ppm 12.85 (s, 1H), 9.04 (s, 1H), 8.25 (s, 1H), 8.03 (d, 1H), 7.87 (t, 1H), 7.79 (d, 1H), 7.61 (d, 1H), 7.54-7.40 (m, 3H), 7.40-7.31 (m, 2H), 7.28 (s, 1H), 7.10 (d, 1H), 7.04 (d, 1H), 6.98 (s, 2H), 6.95 (d, 1H), 6.57 (d, 1H), 6.24-6.11 (m, 1H), 4.96 (s, 2H), 4.86 (t, 1H), 4.65 (d, 2H), 3.95-3.84 (m, 2H), 3.84-3.75 (m, 4H), 3.44-3.24 (m, 10H), 3.01 (t, 2H), 2.62-2.52 (m, 4H), 2.09 (s, 3H), 2.03 (t, 2H), 1.46 (h, 4H), 1.40-1.31 (m, 2H), 1.30-0.88 (m, 14H), 0.87-0.75 (m, 6H). MS (ESI) m/e 1447.5 (M−H)⁻.

### 1.65. Synthesis of 6-[8-(1,3-benzothiazol-2-ylcarbamoyl)-3,4-dihydroisoquinolin-2(1H)-yl]-3-{1-[(3-{2-[(2-carboxyethyl) {[(4-{[(2S,3R,4S,5S,6S)-6-carboxy-3,4,5-trihydroxytetrahydro-2H-pyran-2-yl]oxy}-2-[2-(2-{[3-(2,5-dioxo-2,5-dihydro-1H-pyrrol-1-yl)propanoyl]amino}ethoxy)ethoxy]benzyl)oxy]carbonyl}amino]ethoxy}-5,7-dimethyltricyclo[3.3.1.1^{3,7}]dec-1-yl)methyl]-5-methyl-1H-pyrazol-4-yl}pyridine-2-carboxylic Acid (Synthon II)

### 1.65.1. 3-(1-((3-(2-((((2-(2-(2-aminoethoxy)ethoxy)-4-(((2S,3R,4S,5S,6S)-6-carboxy-3,4,5-trihydroxytetrahydro-2H-pyran-2-yl)oxy)benzyl)oxy)carbonyl)(2-carboxyethyl)amino)ethoxy)-5,7-dimethyladamantan-1-yl)methyl)-5-methyl-1H-pyrazol-4-yl)-6-(8-(benzo[d]thiazol-2-ylcarbamoyl)-3,4-dihydroisoquinolin-2(1H)-yl)picolinic Acid

A solution of Example 1.25.2 (0.055 g,), Example 2.62.6 (0.060 g) and N,N-diisopropylethylamine (0.052 mL) in N,N-dimethylformamide (0.4 mL) as stirred overnight. The reaction was concentrated, and the residue was dissolved in tetrahydrofuran (0.5 mL), methanol (0.5 mL) then treated with lithium hydroxide hydrate (0.037 g) as a solution in water (0.5 mL). After stirring for 1 hour, the reaction was quenched with trifluoroacetic acid (0.091 mL) and diluted with N,N-dimethylformamide (1 mL). The mixture was purified by reverse phase HPLC using a Gilson system, eluting with 10-75% acetonitrile in water containing 0.1% v/v trifluoroacetic acid. The desired fractions were combined and freeze-dried to provide the title compound as the trifluoroacetic acid salt.

### 1.65.2. 6-[8-(1,3-benzothiazol-2-ylcarbamoyl)-3,4-dihydroisoquinolin-2(1H)-yl]-3-{1-[(3-{2-[(2-carboxyethyl) {[(4-{[(2S,3R,4S,5S,6S)-6-carboxy-3,4,5-trihydroxytetrahydro-2H-pyran-2-yl]oxy}-2-[2-(2-{[3-(2,5-dioxo-2,5-dihydro-1H-pyrrol-1-yl)propanoyl]amino}ethoxy)ethoxy]benzyl)oxy]carbonyl}amino]ethoxy}-5,7-dimethyltricyclo[3.3.1.1^{3,7}]dec-1-yl)methyl]-5-methyl-1H-pyrazol-4-yl}pyridine-2-carboxylic Acid

A solution of the trifluoroacetic acid salt of Example 2.65.1 (0.043), 2,5-dioxopyrrolidin-1-yl 3-(2,5-dioxo-2,5-dihydro-1H-pyrrol-1-yl)propanoate (10 mg) and N,N-diisopropylethylamine (0.028 mL) were stirred together in N,N-dimethylformamide (1 mL) at room temperature. After stirring for 1 hour, the reaction was diluted with N,N-dimethylformamide (0.5 mL) and water (0.5 mL). The mixture was purified by reverse phase HPLC using a Gilson system, eluting with 5-75% acetonitrile in water containing 0.1% v/v trifluoroacetic acid. The desired fractions were combined and freeze-dried to provide the title compound. ¹H NMR (400 MHz, dimethyl sulfoxide-d₆) δ ppm 12.84 (s, 1H), 8.03 (d, 1H), 8.00 (t, 1H), 7.79 (d, 1H), 7.62 (d, 1H), 7.54-7.41 (m, 3H), 7.36 (td, 2H), 7.29 (s, 1H), 7.19 (d, 1H), 6.97 (s, 2H), 6.95 (d, 1H), 6.67 (d, 1H), 6.60 (dd, 1H), 5.14-5.03 (m, 1H), 4.96 (d, 4H), 4.08 (tt, 4H), 3.89 (q, 4H), 3.84-3.77 (m, 2H), 3.71 (t, 2H), 3.59 (t, 2H), 3.52-3.35 (m, 6H), 3.28 (dq, 4H), 3.17 (q, 2H), 3.01 (t, 2H), 2.46 (d, 1H), 2.33 (t, 2H), 2.09 (s, 3H), 1.45-0.90 (m, 12H), 0.82 (d, 6H). MS (ESI) m/e 1396.4 (M−H)⁻.

### 1.66. Synthesis of N-[6-(ethenylsulfonyl)hexanoyl]-L-valyl-N-{4-[({[2-({3-[(4-{6-[8-(1,3-benzothiazol-2-ylcarbamoyl)-3,4-dihydroisoquinolin-2(1H)-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](2-sulfoethyl)carbamoyl}oxy)methyl]phenyl}-N⁵-carbamoyl-L-ornithinamide (Synthon KY)

### 1.66.1. 3-(1-((3-(2-((((4-((S)-2-((S)-2-amino-3-methylbutanamido)-5-ureidopentanamido)benzyl)oxy)carbonyl)(2-sulfoethyl)amino)ethoxy)-5,7-dimethyladamantan-1-yl)methyl)-5-methyl-1H-pyrazol-4-yl)-6-(8-(benzo[d]thiazol-2-ylcarbamoyl)-3,4-dihydroisoquinolin-2(1H)-yl)picolinic Acid

To a mixture of Example 1.2.9 (57 mg) and (9H-fluoren-9-yl)methyl ((S)-3-methyl-1-(((S)-1-((4-((((4-nitrophenoxy)carbonyl)oxy)methyl)phenyl)amino)-1-oxo-5-ureidopentan-2-yl)amino)-1-oxobutan-2-yl)carbamate (54 mg) in N,N-dimethylformamide (2 mL) was added N,N-diisopropylethylamine (103 μl). The mixture was stirred overnight, and diethylamine (61.5 μl) was added. The resulting mixture was stirred for 4 hours and purified by reverse phase HPLC using a Gilson system and C18 column, eluting with 10-70% acetonitrile in water containing 0.1% v/v trifluoroacetic acid, to provide the title compound. MS (ESI) m/e 1257.4 (M−H)⁻.

### 1.66.2. N-[6-(ethenylsulfonyl)hexanoyl]-L-valyl-N-{4-[({[2-({3-[(4-{6-[8-(1,3-benzothiazol-2-ylcarbamoyl)-3,4-dihydroisoquinolin-2(1H)-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](2-sulfoethyl)carbamoyl}oxy)methyl]phenyl}-N⁵-carbamoyl-L-ornithinamide

The title compound was prepared using the procedure in Example 2.83, replacing Example 1.2.9 and 2,5-dioxopyrrolidin-1-yl 6-(2-chloroacetamido)hexanoate with Example 2.66.1 and Example 2.82.5, respectively. ¹H NMR (400 MHz, dimethyl sulfoxide-d₆) δ ppm 12.88 (s, OH), 9.99 (s, 1H), 8.05 (t, 2H), 7.80 (t, 2H), 7.60 (q, 3H), 7.36 (td, 2H), 7.28 (d, 3H), 7.01-6.89 (m, 2H), 6.29-6.15 (m, 2H), 6.02 (s, 1H), 4.97 (d, 4H), 4.40 (td, 1H), 4.20 (t, 1H), 4.00-3.77 (m, 4H), 3.55-3.33 (m, 4H), 3.25 (d, 2H), 3.14-2.88 (m, 6H), 2.62 (t, 2H), 2.09 (s, 4H), 1.82-0.90 (m, 10H), 0.84 (dd, 13H). MS (ESI) m/e 1447.2 (M+H).

### 1.67. Synthesis of 4-[(1E)-3-{[(4-{[2-({3-[(4-{6-[8-(1,3-benzothiazol-2-ylcarbamoyl)-3,4-dihydroisoquinolin-2(1H)-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](3-phosphonopropyl)amino}piperidin-1-yl)carbonyl]oxy}prop-1-en-1-yl]-2-({N-[6-(2,5-dioxo-2,5-dihydro-1H-pyrrol-1-yl)hexanoyl]-beta-alanyl}amino)phenyl Beta-D-Glucopyranosiduronic Acid (Synthon IW)

### 1.67.1. 3-(1-((3-(2-((1-((((E)-3-(3-(3-aminopropanamido)-4-(((2S,3R,4S,5S,6S)-6-carboxy-3,4,5-trihydroxytetrahydro-2H-pyran-2-yl)oxy)phenyl)allyl)oxy)carbonyl)piperidin-4-yl) (3-phosphonopropyl)amino)ethoxy)-5,7-dimethyladamantan-1-yl)methyl)-5-methyl-1H-pyrazol-4-yl)-6-(8-(benzo[d]thiazol-2-ylcarbamoyl)-3,4-dihydroisoquinolin-2(1H)-yl)picolinic Acid

To a solution of Example 1.26.2 (0.045 g) and Example 2.44.7 (0.053 g) in N,N-dimethylformamide (1 mL) was added N,N-diisopropylethylamine (0.041 mL), and the reaction was stirred at room temperature overnight. The reaction was concentrated, and the residue was dissolved in methanol (0.5 mL) and tetrahydrofuran (0.5 mL) and treated with a solution of lithium hydroxide monohydrate (0.030 g) in water (0.5 mL) at room temperature. After stirring for 1 hour, the reaction was quenched with trifluoroacetic acid (0.073 mL) and diluted with N,N-dimethylformamide (1 mL). The mixture was purified by reverse phase HPLC using a Gilson system, eluting with 10-60% acetonitrile in water containing 0.1% v/v trifluoroacetic acid. The desired fractions were combined and freeze-dried to provide the title compound.

### 1.67.2. 4-[(1E)-3-{[(4-{[2-({3-[(4-{6-[8-(1,3-benzothiazol-2-ylcarbamoyl)-3,4-dihydroisoquinolin-2(1H)-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](3-phosphonopropyl)amino}piperidin-1-yl)carbonyl]oxy}prop-1-en-1-yl]-2-({N-[6-(2,5-dioxo-2,5-dihydro-1H-pyrrol-1-yl)hexanoyl]-beta-alanyl}amino)phenyl Beta-D-Glucopyranosiduronic Acid

To a solution of Example 2.67.1 (0.040 g) and 2,5-dioxopyrrolidin-1-yl 6-(2,5-dioxo-2,5-dihydro-1H-pyrrol-1-yl)hexanoate (9.84 mg) in N,N-dimethylformamide (1 mL) was added N,N-diisopropylethylamine (0.023 mL), and the reaction was stirred at room temperature for 2 hours. The reaction was diluted with N,N-dimethylformamide (1 mL) and water (1 mL). The mixture was purified by reverse phase HPLC using a Gilson system, eluting with 10-60% acetonitrile in water containing 0.1% v/v trifluoroacetic acid. The desired fractions were combined and freeze-dried to provide the title compound. ¹H NMR (400 MHz, dimethyl sulfoxide-d₆) δ ppm 9.28 (s, 1H), 9.04 (s, 1H), 8.25 (s, 1H), 8.03 (d, 1H), 7.87 (t, 1H), 7.79 (d, 1H), 7.62 (dd, 1H), 7.55-7.40 (m, 3H), 7.36 (td, 2H), 7.29 (s, 1H), 7.11 (dd, 1H), 7.05 (d, 1H), 6.98 (s, 2H), 6.95 (d, 1H), 6.59 (d, 1H), 6.20 (t, 1H), 6.16 (t, 0H), 4.96 (s, 2H), 4.88 (d, 1H), 4.66 (d, 2H), 4.14 (d, 2H), 3.96-3.86 (m, 2H), 3.83 (s, 2H), 3.54 (t, 7H), 3.48-3.28 (m, 12H), 3.01 (t, 2H), 2.84 (s, 2H), 2.55 (t, 2H), 2.10 (s, 3H), 2.07-1.95 (m, 4H), 1.88 (s, 2H), 1.73-1.54 (m, 4H), 1.54-1.38 (m, 6H), 1.39-1.26 (m, 4H), 1.26-0.93 (m, 8H), 0.86 (s, 6H). MS (ESI) m/e 1582.4 (M+H)⁺.

### 1.68. Synthesis of 4-[(1E)-3-{[(4-{[2-({3-[(4-{2-carboxy-6-[8-([1,3]thiazolo[4,5-b]pyridin-2-ylcarbamoyl)-3,4-dihydroisoquinolin-2(1H)-yl]pyridin-3-yl}-5-methyl-1H-pyrazol-1-yl)methyl]-5,7-dimethyltricyclo[3.3.1.1^{3,7}]dec-1-yl}oxy)ethyl](3-phosphonopropyl)amino}piperidin-1-yl)carbonyl]oxy}prop-1-en-1-yl]-2-({N-[6-(2,5-dioxo-2,5-dihydro-1H-pyrrol-1-yl)hexanoyl]-beta-alanyl}amino)phenyl Beta-D-Glucopyranosiduronic Acid (Synthon IY)

### 1.68.1. 3-(1-((3-(2-((1-((((E)-3-(3-(3-aminopropanamido)-4-(((2S,3R,4S,5S,6S)-6-carboxy-3,4,5-trihydroxytetrahydro-2H-pyran-2-yl)oxy)phenyl)allyl)oxy)carbonyl)piperidin-4-yl) (3-phosphonopropyl)amino)ethoxy)-5,7-dimethyladamantan-1-yl)methyl)-5-methyl-1H-pyrazol-4-yl)-6-(8-(thiazolo[4,5-b]pyridin-2-ylcarbamoyl)-3,4-dihydroisoquinolin-2(1H)-yl)picolinic Acid

The title compound was prepared by substituting Example 1.50.2 for Example 1.44.7 in Example 2.56.1. MS (ESI) m/e 1388.5 (M−H)⁻.

### 1.68.2. 4-[(1E)-3-{[(4-{[2-({3-[(4-{2-carboxy-6-[8-([1,3]thiazolo[4,5-b]pyridin-2-ylcarbamoyl)-3,4-dihydroisoquinolin-2(1H)-yl]pyridin-3-yl}-5-methyl-1H-pyrazol-1-yl)methyl]-5,7-dimethyltricyclo[3.3.1.1^{3,7}]dec-1-yl}oxy)ethyl](3-phosphonopropyl)amino}piperidin-1-yl)carbonyl]oxy}prop-1-en-1-yl]-2-({N-[6-(2,5-dioxo-2,5-dihydro-1H-pyrrol-1-yl)hexanoyl]-beta-alanyl}amino)phenyl Beta-D-Glucopyranosiduronic Acid

The title compound was prepared by substituting Example 1.68.1 for Example 1.56.1 in Example 2.56.2. ¹H NMR (400 MHz, dimethyl sulfoxide-d₆) δ ppm 9.03 (s, 1H), 8.61 (d, 1H), 8.50 (d, 1H), 8.25 (br s, 1H), 7.89 (t, 1H), 7.65 (d, 1H), 7.49 (d, 1H), 7.46 (d, 1H), 7.36 (m, 2H), 7.29 (s, 1H), 7.11 (br d, 1H), 7.03 (d, 1H), 6.98 (s, 2H), 6.97 (d, 1H), 6.58 (m, 1H), 6.17 (m, 1H), 4.97 (s, 2H), 4.88 (d, 1H), 4.65 (br d, 2H), 3.88 (m, 3H), 3.79 (br m, 2H), 3.66 (br m, 2H), 3.27-3.44, (m, 14H), 3.01 (m, 2H), 2.85 (br m, 2H), 2.54 (m, 2H), 2.10 (s, 3H), 2.03 (t, 2H), 1.98 (br m, 2H), 1.89 (m, 1H), 1.62 (m, 4H), 1.46 (m, 6H), 1.31 (m, 4H), 1.15 (m, 6H), 1.04 (m, 2H), 0.86 (s, 6H). MS (ESI) m/e 1581.4 (M−H)⁻.

### 1.69. Synthesis of 4-[(1E)-3-({[2-({3-[(4-{6-[8-(1,3-benzothiazol-2-ylcarbamoyl)naphthalen-2-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](2-sulfoethyl)carbamoyl}oxy)prop-1-en-1-yl]-2-({N-[6-(2,5-dioxo-2,5-dihydro-1H-pyrrol-1-yl)hexanoyl]-beta-alanyl}amino)phenyl Beta-D-Glucopyranosiduronic Acid (Synthon JA)

### 1.69.1. 3-(1-((3-(2-(((((E)-3-(3-(3-aminopropanamido)-4-(((2S,3R,4S,5S,6S)-6-carboxy-3,4,5-trihydroxytetrahydro-2H-pyran-2-yl)oxy)phenyl)allyl)oxy)carbonyl)(2-sulfoethyl)amino)ethoxy)-5,7-dimethyladamantan-1-yl)methyl)-5-methyl-1H-pyrazol-4-yl)-6-(8-(benzo[d]thiazol-2-ylcarbamoyl)naphthalen-2-yl)picolinic Acid

The title compound was prepared by substituting Example 1.43.7 for Example 2.44.7 in Example 2.56.1. MS (ESI) m/e 1309.1 (M+Na)⁺.

### 1.69.2. 4-[(1E)-3-({[2-({3-[(4-{6-[8-(1,3-benzothiazol-2-ylcarbamoyl)naphthalen-2-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](2-sulfoethyl)carbamoyl}oxy)prop-1-en-1-yl]-2-({N-[6-(2,5-dioxo-2,5-dihydro-1H-pyrrol-1-yl)hexanoyl]-beta-alanyl}amino)phenyl Beta-D-Glucopyranosiduronic Acid

The title compound was prepared by substituting Example 2.69.1 for Example 2.56.1 in Example 2.56.2. ¹H NMR (400 MHz, dimethyl sulfoxide-d₆) δ ppm 13.09 (s, 1H), 9.02 (s, 2H), 8.35 (d, 1H), 8.13-8.29 (m, 4H), 7.86-8.09 (m, 5H), 7.81 (d, 1H), 7.66-7.75 (m, 1H), 7.44-7.55 (m, 1H), 7.37 (t, 1H), 7.09-7.18 (m, 1H), 7.03 (d, 1H), 6.98 (s, 1H), 6.48-6.62 (m, 1H), 6.07-6.22 (m, 1H), 4.81-4.92 (m, 1H), 4.58-4.74 (m, 2H), 3.80-3.93 (m, 3H), 3.27-3.37 (m, 5H), 2.53-2.68 (m, 4H), 2.15-2.23 (m, 3H), 2.03 (t, 2H), 1.36-1.53 (m, 6H), 0.97-1.33 (m, 24H), 0.81 (d, 6H). MS (ESI) m/e 1478.3 (M−H)⁻.

### 1.70. This Paragraph was Intentionally Left Blank

### 1.71. This Paragraph was Intentionally Left Blank

### 1.72. This Paragraph was Intentionally Left Blank

### 1.73. This Paragraph was Intentionally Left Blank

### 1.74. This Paragraph was Intentionally Left Blank

### 1.75. This Paragraph was Intentionally Left Blank

### 1.76. This Paragraph was Intentionally Left Blank

### 1.77. Synthesis of 6-[8-(1,3-benzothiazol-2-ylcarbamoyl)-3,4-dihydroisoquinolin-2(1H)-yl]-3-[1-({3-[2-({N-[6-(2,5-dioxo-2,5-dihydro-1H-pyrrol-1-yl)hexanoyl]-3-sulfo-L-alanyl}amino)ethoxy]-5,7-dimethyltricyclo[3.3.1.1^{3,7}]dec-1-yl}methyl)-5-methyl-1H-pyrazol-4-yl]pyridine-2-carboxylic Acid (Synthon FA)

To a solution of Example 1.15 (0.023 g) and 2,5-dioxopyrrolidin-1-yl 6-(2,5-dioxo-2,5-dihydro-1H-pyrrol-1-yl)hexanoate (9.12 mg) in N,N-dimethylformamide (0.5 mL) was added N,N-diisopropylethylamine (0.012 mL), and the reaction was stirred overnight. The reaction was diluted with N,N-dimethylformamide (1 mL) and water (0.5 mL). The mixture was purified by reverse phase HPLC using a Gilson system, eluting with 10-85% acetonitrile in water containing 0.1% v/v trifluoroacetic acid. The desired fractions were combined and freeze-dried to provide the title compound. ¹H NMR (400 MHz, dimethyl sulfoxide-d₆) δ ppm 12.84 (s, 1H), 8.04 (d, 1H), 7.90 (d, 1H), 7.79 (d, 1H), 7.65-7.57 (m, 2H), 7.54 (d, 1H), 7.51-7.41 (m, 2H), 7.40-7.31 (m, 3H), 7.01-6.96 (m, 3H), 4.96 (s, 2H), 4.34-4.28 (m, 3H), 3.89 (t, 2H), 3.83 (s, 2H), 3.37 (t, 2H), 3.29 (t, 2H), 3.16-2.95 (m, 4H), 2.80 (dd, 1H), 2.70 (dd, 1H), 2.11 (s, 3H), 2.06 (t, 2H), 1.47 (tt, 4H), 1.40-0.92 (m, 12H), 0.84 (s, 6H). MS (ESI) m/e 1090.3 (M+H)⁺.

### 1.78. Synthesis of 6-[8-(1,3-benzothiazol-2-ylcarbamoyl)-3,4-dihydroisoquinolin-2(1H)-yl]-3-[1-({3-[2-(2-{[6-(2,5-dioxo-2,5-dihydro-1H-pyrrol-1-yl)hexanoyl](2-sulfoethyl)amino}ethoxy)ethoxy]-5,7-dimethyltricyclo[3.3.1.1^{3,7}]dec-1-yl}methyl)-5-methyl-1H-pyrazol-4-yl]pyridine-2-carboxylic Acid (Synthon FJ)

The title compound was prepared as described in Example 2.1, replacing Example 1.2.9 and 4-((S)-2-((S)-2-(6-(2,5-dioxo-2,5-dihydro-1H-pyrrol-1-yl) hexanamido)-3-methylbutanamido)-5-ureidopentanamido)benzyl (4-nitrophenyl) carbonate with Example 1.11.4 and perfluorophenyl 6-(2,5-dioxo-2,5-dihydro-1H-pyrrol-1-yl)hexanoate, respectively. ¹H NMR (400 MHz, dimethyl sulfoxide-d₆) δ ppm 12.84 (s, 1H), 8.04 (d, 1H), 7.79 (d, 1H), 7.61 (d, 1H), 7.52 (dd, 1H), 7.42-7.49 (m, 2H), 7.33-7.39 (m, 2H), 7.30 (s, 1H), 6.98 (s, 2H), 6.96 (d, 1H), 4.95 (s, 2H), 3.89 (t, 2H), 3.82 (s, 2H), 3.46-3.56 (m, 4H), 3.31-3.46 (m, 10H), 3.01 (t, 2H), 2.61-2.68 (m, 1H), 2.55-2.60 (m, 1H), 2.21-2.32 (m, 2H), 2.10 (s, 3H), 1.40-1.51 (m, 4H), 1.37 (d, 2H), 0.91-1.30 (m, 12H), 0.83 (s, 6H). MS (ESI) m/e 1091.2 (M+H)⁺.

### 1.79. Synthesis of 6-[8-(1,3-benzothiazol-2-ylcarbamoyl)-3,4-dihydroisoquinolin-2(1H)-yl]-3-(1-{[3-(2-{[6-(2,5-dioxo-2,5-dihydro-1H-pyrrol-1-yl)hexanoyl](2-sulfoethyl)amino}ethoxy)-5,7-dimethyltricyclo[3.3.1.1^{3,7}]dec-1-yl]methyl}-5-methyl-1H-pyrazol-4-yl)pyridine-2-carboxylic Acid (Synthon FK)

The title compound was prepared as described in Example 2.1, replacing 4-((S)-2-((S)-2-(6-(2,5-dioxo-2,5-dihydro-1H-pyrrol-1-yl) hexanamido)-3-methylbutanamido)-5-ureidopentanamido)benzyl (4-nitrophenyl) carbonate with perfluorophenyl 6-(2,5-dioxo-2,5-dihydro-1H-pyrrol-1-yl)hexanoate. ¹H NMR (400 MHz, dimethyl sulfoxide-d₆) δ ppm 12.85 (s, 1H), 8.04 (d, 1H), 7.79 (d, 1H), 7.61 (d, 1H), 7.52 (dd, 1H), 7.41-7.49 (m, 2H), 7.32-7.39 (m, 2H), 7.28 (s, 1H), 6.93-6.98 (m, 3H), 4.95 (s, 2H), 3.89 (t, 2H), 3.81 (s, 2H), 3.32-3.38 (m, 2H), 3.21-3.27 (m, 2H), 3.01 (t, 2H), 2.61-2.67 (m, 2H), 2.53-2.58 (m, 2H), 2.33-2.39 (m, 1H), 2.20-2.29 (m, 2H), 2.09 (s, 3H), 1.40-1.51 (m, 4H), 1.34 (s, 2H), 0.93-1.27 (m, 13H), 0.83 (s, 6H). MS (ESI) m/e 1047.2 (M+H)⁺.

### 1.80. Synthesis of 6-[8-(1,3-benzothiazol-2-ylcarbamoyl)-3,4-dihydroisoquinolin-2(1H)-yl]-3-{1-[(3-{[1-(2,5-dioxo-2,5-dihydro-1H-pyrrol-1-yl)-21-oxo-22-(2-sulfoethyl)-3,6,9,12,15,18-hexaoxa-22-azatetracosan-24-yl]oxy}-5,7-dimethyltricyclo[3.3.1.1^{3,7}]dec-1-yl)methyl]-5-methyl-1H-pyrazol-4-yl}pyridine-2-carboxylic Acid (Synthon FQ)

The title compound was prepared as described in Example 2.1, replacing 4-((S)-2-((S)-2-(6-(2,5-dioxo-2,5-dihydro-1H-pyrrol-1-yl) hexanamido)-3-methylbutanamido)-5-ureidopentanamido)benzyl (4-nitrophenyl) carbonate with perfluorophenyl 1-(2,5-dioxo-2,5-dihydro-1H-pyrrol-1-yl)-3,9,12,15,18-pentaoxahenicosan-21-oate. ¹H NMR (400 MHz, dimethyl sulfoxide-d₆) δ ppm 12.84 (s, 1H), 8.04 (d, 1H), 7.79 (d, 1H), 7.61 (d, 1H), 7.42-7.54 (m, 3H), 7.33-7.38 (m, 2H), 7.28 (s, 1H), 6.95 (dd, 1H), 4.95 (s, 2H), 3.89 (t, 2H), 3.81 (s, 2H), 3.07-3.53 (m, 24H), 3.01 (t, 2H), 2.61-2.69 (m, 1H), 2.54-2.60 (m, 1H), 2.09 (s, 3H), 1.96 (d, 2H), 0.92-1.39 (m, 13H), 0.84 (s, 6H). MS (ESI) m/e 1269.4 (M+H)⁺.

### 1.81. Synthesis of 6-[8-(1,3-benzothiazol-2-ylcarbamoyl)-3,4-dihydroisoquinolin-2(1H)-yl]-3-{1-[(3-{[1-(2,5-dioxo-2,5-dihydro-1H-pyrrol-1-yl)-21-oxo-22-(2-sulfoethyl)-3,6,9,12,15,18,25-heptaoxa-22-azaheptacosan-27-yl]oxy}-5,7-dimethyltricyclo[3.3.1.1^{3,7}]dec-1-yl)methyl]-5-methyl-1H-pyrazol-4-yl}pyridine-2-carboxylic Acid (Synthon FR)

The title compound was prepared as described in Example 2.1, replacing Example 1.2.9 and 4-((S)-2-((S)-2-(6-(2,5-dioxo-2,5-dihydro-1H-pyrrol-1-yl) hexanamido)-3-methylbutanamido)-5-ureidopentanamido)benzyl (4-nitrophenyl) carbonate with Example 1.11.4 and perfluorophenyl 1-(2,5-dioxo-2,5-dihydro-1H-pyrrol-1-yl)-3,6,9,12,15,18-hexaoxahenicosan-21-oate, respectively. ¹H NMR (500 MHz, dimethyl sulfoxide-d₆) δ ppm 12.84 (s, 1H), 8.04 (d, 1H), 7.79 (d, 1H), 7.61 (d, 1H), 7.52 (d, 1H), 7.41-7.50 (m, 2H), 7.33-7.39 (m, 2H), 7.31 (s, 1H), 7.01 (d, 2H), 6.97 (d, 1H), 4.96 (s, 2H), 3.89 (t, 2H), 3.83 (s, 2H), 3.31-3.60 (m, 30H), 3.01 (t, 2H), 2.64-2.71 (m, 1H), 2.53-2.61 (m, 3H), 2.10 (s, 3H), 1.38 (s, 2H), 1.20-1.31 (m, 4H), 1.12-1.18 (m, 2H), 0.91-1.12 (m, 4H), 0.84 (s, 6H).

### 1.82. Synthesis of 6-[8-(1,3-benzothiazol-2-ylcarbamoyl)-3,4-dihydroisoquinolin-2(1H)-yl]-3-(1-{[3-(2-{[6-(ethenylsulfonyl)hexanoyl](2-sulfoethyl)amino}ethoxy)-5,7-dimethyltricyclo[3.3.1.1^{3,7}]dec-1-yl]methyl}-5-methyl-1H-pyrazol-4-yl)pyridine-2-carboxylic Acid (Synthon JE)

### 1.82.1. Ethyl 6-((2-hydroxyethyl)thio)hexanoate

A mixture of ethyl 6-bromohexanoate (3 g), 2-mercaptoethanol (0.947 mL) and K₂CO₃ (12 g) in ethanol (100 mL) was stirred overnight and filtered. The filtrate was concentrated. The residue was dissolved in dichloromethane (100 mL) and washed with water and brine. The organic layer was dried over sodium sulfate, filtered, and concentrated to provide the title compound.

### 1.82.2. 6-((2-hydroxyethyl)thio)hexanoic Acid

A mixture of Example 2.82.1 (12 g) and 3 M aqueous NaOH solution (30 mL) in ethanol (30 mL) was stirred overnight. The organics were removed under reduced pressure. The residual aqueous phase was washed with ethyl acetate, acidified with HCl to pH 5 and extracted with dichloromethane. The extracts were combined, dried over sodium sulfate, filtered and concentrated to provide the title compound.

### 1.82.3. 6-((2-hydroxyethyl)sulfonyl)hexanoic Acid

To a stirred solution of Example 2.82.2 (4 g) in a mixture of water (40 mL) and 1,4-dioxane (160 mL) was added Oxone® (38.4 g), and the mixture was stirred overnight. The mixture was filtered, and the filtrate was concentrated. The residual aqueous layer was extracted with dichloromethane. The extracts were combined and dried over sodium sulfate, filtered, and concentrated to provide the title compound.

### 1.82.4. 6-(vinylsulfonyl)hexanoic Acid

To a cold (0° C.) solution of Example 2.82.3 (1 g) in dichloromethane (10 mL) was added triethylamine (2.8 mL), followed by the addition of methanesulfonyl chloride (1.1 mL) under argon. The mixture was stirred overnight and washed with water and brine. The organic layer was dried over sodium sulfate, filtered, and concentrated to provide the title compound.

### 1.82.5. 2,5-dioxopyrrolidin-1-yl 6-(vinylsulfonyl)hexanoate

To a stirred solution of Example 2.82.4 (0.88 g) in dichloromethane (10 ml) was added 1-hydroxypyrrolidine-2,5-dione (0.54 g) and N,N′-methanediylidenedicyclohexanamine (0.92 g). The mixture was stirred overnight and filtered. The filtrate was concentrated and purified by flash chromatography, eluting with 10-25% ethyl acetate in petroleum, to provide the title compound. MS (ESI) m/e 304.1 (M+1).

### 1.82.6. 6-[8-(1,3-benzothiazol-2-ylcarbamoyl)-3,4-dihydroisoquinolin-2(1H)-yl]-3-(1-{[3-(2-{[6-(ethenylsulfonyl)hexanoyl](2-sulfoethyl)amino}ethoxy)-5,7-dimethyltricyclo[3.3.1.1^{3,7}]dec-1-yl]methyl}-5-methyl-1H-pyrazol-4-yl)pyridine-2-carboxylic Acid

The title compound was prepared as described in Example 2.83, replacing 2,5-dioxopyrrolidin-1-yl 6-(2-chloroacetamido)hexanoate with Example 2.82.5. ¹H NMR (400 MHz, dimethyl sulfoxide-d₆) δ ppm 12.86 (s, 1H), 8.04 (d, 1H), 7.79 (d, 1H), 7.61 (d, 1H), 7.53 (dd, 1H), 7.42-7.49 (m, 2H), 7.33-7.40 (m, 2H), 7.28 (s, 1H), 6.88-7.00 (m, 2H), 6.17-6.25 (m, 2H), 4.95 (s, 2H), 3.89 (t, 2H), 3.81 (s, 2H), 3.38 (dd, 2H), 3.25 (t, 2H), 3.04-3.12 (m, 2H), 3.01 (t, 2H), 2.62-2.69 (m, 1H), 2.56 (dd, 1H), 2.27 (q, 2H), 2.09 (s, 3H), 1.53-1.62 (m, 2H), 1.43-1.51 (m, 2H), 1.28-1.38 (m, 4H), 1.20-1.27 (m, 4H), 0.92-1.19 (m, 6H), 0.84 (s, 6H). MS (ESI) m/e 1042.2 (M+H)⁺.

### 1.83. Synthesis of 6-[8-(1,3-benzothiazol-2-ylcarbamoyl)-3,4-dihydroisoquinolin-2(1H)-yl]-3-{1-[(3-{2-[{6-[(chloroacetyl)amino]hexanoyl}(2-sulfoethyl)amino]ethoxy}-5,7-dimethyltricyclo[3.3.1.1^{3,7}]dec-1-yl)methyl]-5-methyl-1H-pyrazol-4-yl}pyridine-2-carboxylic Acid (Synthon JM)

To a mixture of Example 1.2.9 (12.5 mg) and 2,5-dioxopyrrolidin-1-yl 6-(2-chloroacetamido)hexanoate (6.7 mg) in N,N-dimethylformamide (1.5 mL) was added N,N-diisopropylethylamine (26 μL). The mixture was stirred for 10 days and purified by reverse phase HPLC using a Gilson system and C18 column, eluting with 20-60% acetonitrile in water containing 0.1% v/v trifluoroacetic acid, to provide the title compound. ¹H NMR (500 MHz, dimethyl sulfoxide-d₆) δ ppm 12.83 (s, 1H), 8.15-8.21 (m, 1H), 8.04 (d, 1H), 7.79 (d, 1H), 7.61 (d, 1H), 7.52 (dd, 1H), 7.41-7.49 (m, 2H), 7.32-7.39 (m, 2H), 7.28 (s, 1H), 6.96 (dd, 1H), 4.95 (s, 2H), 4.01 (d, 2H), 3.89 (t, 2H), 3.81 (s, 2H), 3.39 (d, 2H), 3.25 (t, 2H), 2.98-3.10 (m, 5H), 2.62-2.70 (m, 1H), 2.56-2.61 (m, 1H), 2.23-2.30 (m, 2H), 2.09 (s, 3H), 1.33-1.52 (m, 5H), 1.19-1.30 (m, 6H), 0.91-1.18 (m, 6H), 0.84 (s, 6H). MS (ESI) m/e 1043.2 (M+H)⁺.

### 1.84. Synthesis of N-[6-(2,5-dioxo-2,5-dihydro-1H-pyrrol-1-yl)hexanoyl]-L-valyl-N-{4-[({[2-({3-[(4-{6-[8-(1,3-benzothiazol-2-ylcarbamoyl)-3,4-dihydroisoquinolin-2(1H)-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](3-carboxypropyl) carbamoyl}oxy)methyl]phenyl}-N⁵-carbamoyl-L-ornithinamide (Synthon LE)

A mixture of Example 1.56 (0.020 g), 4-((S)-2-((S)-2-(6-(2,5-dioxo-2,5-dihydro-1H-pyrrol-1-yl) hexanamido)-3-methylbutanamido)-5-ureidopentanamido)benzyl (4-nitrophenyl) carbonate (0.022 g) and N,N-diisopropylethylamine (0.018 mL) were stirred together in N,N-dimethylformamide (0.4 mL) at room temperature. After stirring for 5 hours, the reaction was diluted with a 1:1 mixture of N,N-dimethylformamide and water (2 mL). The mixture was purified by reverse phase HPLC using a Gilson system, eluting with 10-85% acetonitrile in water containing 0.1% v/v trifluoroacetic acid. The desired fractions were combined and freeze-dried to provide the title compound. ¹H NMR (400 MHz, dimethyl sulfoxide-d₆) δ ppm 12.82 (s, 1H), 9.97 (s, 1H), 8.10-7.98 (m, 2H), 7.84-7.72 (m, 2H), 7.67-7.54 (m, 3H), 7.54-7.41 (m, 3H), 7.40-7.32 (m, 2H), 7.30-7.23 (m, 3H), 6.99 (s, 2H), 6.94 (d, 1H), 5.99 (s, 1H), 4.98 (s, 2H), 4.95 (s, 2H), 4.45-4.35 (m, 2H), 4.19 (dd, 2H), 3.88 (t, 2H), 3.82-3.76 (m, 2H), 3.47-3.31 (m, 4H), 3.28-3.19 (m, 4H), 3.07-2.89 (m, 4H), 2.21-2.11 (m, 4H), 2.09 (s, 2H), 2.02-1.89 (m, 1H), 1.77-1.63 (m, 2H), 1.62-1.27 (m, 10H), 1.27-0.90 (m, 13H), 0.88-0.78 (m, 12H); MS (ESI) m/e 1430.3 (M+1)⁺.

### 1.85. Synthesis of N-{6-[(bromoacetyl)amino]hexanoyl}-L-valyl-N-{4-[({[2-({3-[(4-{6-[8-(1,3-benzothiazol-2-ylcarbamoyl)-3,4-dihydroisoquinolin-2(1H)-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](2-sulfoethyl)carbamoyl}oxy)methyl]phenyl}-N⁵-carbamoyl-L-ornithinamide (Synthon LH)

### 1.85.1. 1H-benzo[d][1,2,3]triazol-1-yl 6-(2-bromoacetamido)hexanoate

To a solution of 6-(2-bromoacetamido)hexanoic acid (105 mg) and benzotriazol-1-yloxy)tripyrrolidinophosphonium hexafluorophosphate (PyBOP, 325 mg) in N,N-dimethylformamide (3 mL) was added triethylamine (87 μl). The mixture was stirred for 1 hour and purified by a Gilson HPLC system (C18 column), eluting with 20-60% acetonitrile in 0.1% TFA water to provide the title compound. MS (ESI) m/e 368.7 (M+H).

### 1.85.2. N-{6-[(bromoacetyl)amino]hexanoyl}-L-valyl-N-{4-[({[2-({3-[(4-{6-[8-(1,3-benzothiazol-2-ylcarbamoyl)-3,4-dihydroisoquinolin-2(1H)-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](2-sulfoethyl)carbamoyl}oxy)methyl]phenyl}-N⁵-carbamoyl-L-ornithinamide

To a mixture of Example 2.66.1 (6.6 mg) and Example 2.85.2 (3.6 mg) in N,N-dimethylformamide (0.3 mL) was added N,N-diisopropylethylamine (2.52 μl). The mixture was stirred for 5 minutes, diluted with dimethyl sulfoxide and purified by reverse phase HPLC using a Gilson system and C18 column, eluting with 20-60% acetonitrile in water containing 0.1% v/v trifluoroacetic acid, to provide the title compound. ¹H NMR (500 MHz, dimethyl sulfoxide-d₆) δ 9.99 (s, 1H), 8.24 (s, 1H), 8.08 (d, 1H), 8.04 (d, 1H), 7.80 (dd, 2H), 7.60 (q, 3H), 7.56-7.50 (m, 1H), 7.50-7.41 (m, 2H), 7.36 (q, 2H), 7.32-7.25 (m, 3H), 6.96 (d, 1H), 4.98 (d, 4H), 4.39 (q, 1H), 4.20 (dd, 1H), 3.92-3.68 (m, 6H), 3.42 (dd, 1H), 3.25 (t, 2H), 3.09-2.87 (m, 6H), 2.64 (s, 2H), 2.25-1.87 (m, 5H), 1.79-0.89 (m, 17H), 0.88-0.67 (m, 12H). MS (ESI) m/e 1492.5 (M−H)⁻.

### 1.86. Synthesis of 4-[({[2-({3-[(4-{6-[8-(1,3-benzothiazol-2-ylcarbamoyl)-3,4-dihydroisoquinolin-2(1H)-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](3-carboxypropyl) carbamoyl}oxy)methyl]-3-[2-(2-{[3-(2,5-dioxo-2,5-dihydro-1H-pyrrol-1-yl)propanoyl]amino}ethoxy)ethoxy]phenyl Beta-D-Glucopyranosiduronic Acid (Synthon LJ)

### 1.86.1. 3-(1-((3-(2-((((2-(2-(2-aminoethoxy)ethoxy)-4-(((2S,3R,4S,5S,6S)-6-carboxy-3,4,5-trihydroxytetrahydro-2H-pyran-2-yl)oxy)benzyl)oxy)carbonyl)(3-carboxypropyl)amino)ethoxy)-5,7-dimethyladamantan-1-yl)methyl)-5-methyl-1H-pyrazol-4-yl)-6-(8-(benzo[d]thiazol-2-ylcarbamoyl)-3,4-dihydroisoquinolin-2(1H)-yl)picolinic Acid

To a solution of Example 1.56 (0.024 g) and Example 2.62.6 (0.030 g) in N,N-dimethylformamide (0.4 mL) was added N,N-diisopropylethylamine (0.025 mL), and the reaction was stirred overnight. The reaction was concentrated, and the residue dissolved in tetrahydrofuran (0.5 mL) and methanol (0.5 mL) and treated with lithium hydroxide hydrate (0.018 g) as a solution in water (0.5 mL). After stirring for 1 hour, the reaction was diluted with N,N-dimethylformamide (1 mL) and purified by reverse phase HPLC using a Gilson system, eluting with 10-75% acetonitrile in water containing 0.1% v/v trifluoroacetic acid. The desired fractions were combined and freeze-dried to provide the title compound. MS (ESI) m/e 1262.7 (M+H)⁺.

### 1.86.2. 4-[({[2-({3-[(4-{6-[8-(1,3-benzothiazol-2-ylcarbamoyl)-3,4-dihydroisoquinolin-2(1H)-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](3-carboxypropyl) carbamoyl}oxy)methyl]-3-[2-(2-{[3-(2,5-dioxo-2,5-dihydro-1H-pyrrol-1-yl)propanoyl]amino}ethoxy)ethoxy]phenyl Beta-D-Glucopyranosiduronic Acid

To a solution of Example 2.86.1 (0.0173 g) and 2,5-dioxopyrrolidin-1-yl 3-(2,5-dioxo-2,5-dihydro-1H-pyrrol-1-yl)propanoate (4.38 mg) in N,N-dimethylformamide (0.8 mL) was added 2,5-dioxopyrrolidin-1-yl 3-(2,5-dioxo-2,5-dihydro-1H-pyrrol-1-yl)propanoate (4.38 mg), and the reaction was stirred for 2 hours. The reaction was diluted with a 1:1 mixture of N,N-dimethylformamide:water (1 mL), and the mixture was purified by reverse phase HPLC using a Gilson system, eluting with 10-80% acetonitrile in water containing 0.1% v/v trifluoroacetic acid. The desired fractions were combined and freeze-dried to provide the title compound. ¹H NMR (400 MHz, dimethyl sulfoxide-d₆) δ ppm 12.77 (s, 1H), 8.03 (d, 1H), 7.99 (t, 1H), 7.77 (d, 1H), 7.62 (d, 1H), 7.55-7.41 (m, 3H), 7.40-7.32 (m, 2H), 8.28 (s, 1H), 7.23-7.17 (m, 1H), 6.97 (s, 2H), 6.94 (d, 1H), 6.66 (s, 1H), 6.60 (dd, 1H), 5.07 (m, 1H), 5.00-4.91 (m, 4H), 4.17-4.02 (m, 2H), 3.96-3.85 (m, 2H), 3.85-3.76 (m, 2H), 3.71 (t, 2H), 3.64-3.56 (m, 4H), 3.34-3.12 (m, 10H), 3.01 (, 2H), 2.33 (t, 2H), 2.24-2.12 (m, 2H), 2.09 (s, 3H), 1.70 (p, 2H), 1.45-0.88 (m, 12H), 0.88-0.77 (m, 6H); MS (ESI) m/e 1434.2 (M+Na)⁺.

### 1.87. Synthesis of 4-({[(4-{[2-({3-[(4-{6-[8-(1,3-benzothiazol-2-ylcarbamoyl)-3,4-dihydroisoquinolin-2(1H)-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](3-carboxypropyl)amino}piperidin-1-yl)carbonyl]oxy}methyl)-3-[2-(2-{[3-(2,5-dioxo-2,5-dihydro-1H-pyrrol-1-yl)propanoyl]amino}ethoxy)ethoxy]phenyl Beta-D-Glucopyranosiduronic Acid (Synthon MA)

### 1.87.1. 3-(1-((3-(2-((1-(((2-(2-(2-aminoethoxy)ethoxy)-4-(((2S,3R,4S,5S,6S)-6-carboxy-3,4,5-trihydroxytetrahydro-2H-pyran-2-yl)oxy)benzyl)oxy)carbonyl)piperidin-4-yl) (3-carboxypropyl)amino)ethoxy)-5,7-dimethyladamantan-1-yl)methyl)-5-methyl-1H-pyrazol-4-yl)-6-(8-(benzo[d]thiazol-2-ylcarbamoyl)-3,4-dihydroisoquinolin-2(1H)-yl)picolinic Acid

A solution of Example 1.42 (0.050 g) and Example 2.62.6 (0.050 g) in N,N-dimethylformamide (0.5 mL) was treated with N,N-diisopropylethylamine (0.042 mL), and the reaction was stirred at room temperature for 2 hours. The reaction was concentrated, and the residue was dissolved in methanol (0.5 mL) and tetrahydrofuran (0.5 mL) and treated with lithium hydroxide hydrate (0.031 g) as a solution in water (0.5 mL). The reaction was stirred for 1.5 hours and diluted with N,N-dimethylformamide (1 mL). The mixture was purified by reverse phase HPLC using a Gilson system, eluting with 10-80% acetonitrile in water containing 0.1% v/v trifluoroacetic acid. The desired fractions were combined and freeze-dried to provide the title compound. MS (ESI) m/e 1345.7 (M+H)⁺.

### 1.87.2. 4-({[(4-{[2-({3-[(4-{6-[8-(1,3-benzothiazol-2-ylcarbamoyl)-3,4-dihydroisoquinolin-2(1H)-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](3-carboxypropyl)amino}piperidin-1-yl)carbonyl]oxy}methyl)-3-[2-(2-{[3-(2,5-dioxo-2,5-dihydro-1H-pyrrol-1-yl)propanoyl]amino}ethoxy)ethoxy]phenyl Beta-D-Glucopyranosiduronic Acid

A solution of Example 2.87.1 (0.047 g) and 2,5-dioxopyrrolidin-1-yl 3-(2,5-dioxo-2,5-dihydro-1H-pyrrol-1-yl)propanoate (0.011 g) in N,N-dimethylformamide (0.5 mL) was treated with N,N-diisopropylethylamine (0.031 mL), and the reaction was stirred at room temperature for 2 hours. The reaction was diluted with a 1:1 mixture of N,N-dimethylformamide:water (2 mL). The mixture was purified by reverse phase HPLC using a Gilson system, eluting with 10-85% acetonitrile in water containing 0.1% v/v trifluoroacetic acid. The desired fractions were combined and freeze-dried to provide the title compound. ¹H NMR (400 MHz, dimethyl sulfoxide-d₆) δ ppm 12.87 (s, 1H), 8.96 (s, 1H), 8.15-8.07 (m, 2H), 7.88 (d, J=8.1 Hz, 1H), 7.71 (d, J=7.5 Hz, 1H), 7.62-7.50 (m, 3H), 7.50-7.45 (m, 1H), 7.45-7.42 (m, 1H), 7.37 (s, 1H), 7.33-7.27 (m, 1H), 7.07 (s, 2H), 7.07-7.02 (m, 1H), 6.80-6.74 (m, 1H), 6.72-6.66 (m, 1H), 5.23-5.14 (m, 1H), 5.13-5.00 (m, 4H), 4.27-4.12 (m, 4H), 4.06-3.95 (m, 4H), 3.92 (s, 2H), 3.83-3.78 (m, 2H), 3.57-3.32 (m, 10H), 3.32-3.14 (m, 4H), 3.14-3.06 (m, 2H), 2.90 (s, 2H), 2.49-2.37 (m, 4H), 2.19 (s, 3H), 2.12-2.01 (m, 2H), 2.02-1.88 (m, 2H), 1.74-1.57 (m, 2H), 1.52 (s, 2H), 1.45-1.30 (m, 4H), 1.30-1.05 (m, 6H), 0.95 (s, 6H); MS (ESI) m/e 1495.4 (M+H)⁺.

### 1.88. Synthesis of 4-[({[2-({3-[(4-{6-[8-(1,3-benzothiazol-2-ylcarbamoyl)-3,4-dihydroisoquinolin-2(1H)-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](3-sulfopropyl) carbamoyl}oxy)methyl]-3-[2-(2-{[3-(2,5-dioxo-2,5-dihydro-1H-pyrrol-1-yl)propanoyl]amino}ethoxy)ethoxy]phenyl Beta-D-Glucopyranosiduronic Acid (Synthon MD)

### 1.88.1. 3-(1-((3-(2-((((2-(2-(2-aminoethoxy)ethoxy)-4-(((2S,3R,4S,5S,6S)-6-carboxy-3,4,5-trihydroxytetrahydro-2H-pyran-2-yl)oxy)benzyl)oxy)carbonyl)(3-sulfopropyl)amino)ethoxy)-5,7-dimethyladamantan-1-yl)methyl)-5-methyl-1H-pyrazol-4-yl)-6-(8-(benzo[d]thiazol-2-ylcarbamoyl)-3,4-dihydroisoquinolin-2(1H)-yl)picolinic Acid

A solution of Example 1.6 (0.039 g) and Example 2.62.6 (0.041 g) in N,N-dimethylformamide (0.5 mL) was treated with N,N-diisopropylethylamine (0.035 mL), and the reaction was stirred at room temperature for 2 hours. The reaction was concentrated, and the residue was dissolved in methanol (0.5 mL) and tetrahydrofuran (0.5 mL) and treated with lithium hydroxide hydrate (0.025 g) as a solution in water (0.5 mL). The reaction was stirred for 1.5 hours and diluted with N,N-dimethylformamide (1 mL). The mixture was purified by reverse phase HPLC using a Gilson system, eluting with 10-80% acetonitrile in water containing 0.1% v/v trifluoroacetic acid. The desired fractions were combined and freeze-dried to provide the title compound. MS (ESI) m/e 1297.8 (M+H)⁺.

### 1.88.2. 4-[({[2-({3-[(4-{6-[8-(1,3-benzothiazol-2-ylcarbamoyl)-3,4-dihydroisoquinolin-2(1H)-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](3-sulfopropyl) carbamoyl}oxy)methyl]-3-[2-(2-{[3-(2,5-dioxo-2,5-dihydro-1H-pyrrol-1-yl)propanoyl]amino}ethoxy)ethoxy]phenyl Beta-D-Glucopyranosiduronic Acid

To a solution of Example 2.88.1 (0.024 g) and 2,5-dioxopyrrolidin-1-yl 3-(2,5-dioxo-2,5-dihydro-1H-pyrrol-1-yl)propanoate (6.40 mg) in N,N-dimethylformamide (0.5 mL) was added N,N-diisopropylethylamine (0.016 mL), and the reaction was stirred at room temperature for 1 hour. The reaction was diluted with a 1:1 mixture of N,N-dimethylformamide:water (2 mL). The mixture was purified by reverse phase HPLC using a Gilson system, eluting with 10-80% acetonitrile in water containing 0.1% v/v trifluoroacetic acid. The desired fractions were combined and freeze-dried to provide the title compound. ¹H NMR (500 MHz, dimethyl sulfoxide-d₆) δ ppm 12.87 (s, 1H), 8.09-8.02 (m, 2H), 7.79 (d, 1H), 7.61 (d, 1H), 7.52 (dd, 1H), 7.50-7.42 (m, 2H), 7.40-7.33 (m, 2H), 7.31 (s, 1H), 7.20 (t, 1H), 6.98 (s, 3H), 6.66 (s, 1H), 6.60 (dd, 1H), 5.06 (t, 1H), 4.96 (s, 4H), 4.10 (dq, 4H), 3.81 (d, 4H), 3.71 (t, 2H), 3.59 (t, 2H), 3.51-3.35 (m, 4H), 3.26 (td, 6H), 3.17 (q, 2H), 3.01 (t, 2H), 2.35 (dt, 4H), 2.10 (d, 3H), 1.75 (d, 2H), 1.44-0.88 (m, 12H), 0.82 (d, 6H); MS (ESI) m/e 1446.4 (M−H)⁻.

### 1.89. Synthesis of N-[6-(2,5-dioxo-2,5-dihydro-1H-pyrrol-1-yl)hexanoyl]-L-valyl-N-[4-({[(3-{[2-({3-[(4-{6-[8-(1,3-benzothiazol-2-ylcarbamoyl)-3,4-dihydroisoquinolin-2(1H)-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](2-sulfoethyl)amino}azetidin-1-yl)carbonyl]oxy}methyl)phenyl]-N⁵-carbamoyl-L-ornithinamide (Synthon MG)

A solution of Example 1.60 (0.026 g), 4-((S)-2-((S)-2-(6-(2,5-dioxo-2,5-dihydro-1H-pyrrol-1-yl) hexanamido)-3-methylbutanamido)-5-ureidopentanamido)benzyl (4-nitrophenyl) carbonate (0.024 g) and N,N-diisopropylethylamine (0.022 mL) were stirred together in N,N-dimethylformamide (0.8 mL) at room temperature for 3 hours. The reaction was diluted with a 1:1 mixture of N,N-dimethylformamide:water (2 mL). The mixture was purified by reverse phase HPLC using a Gilson system, eluting with 10-80% acetonitrile in water containing 0.1% v/v trifluoroacetic acid. The desired fractions were combined and freeze-dried to provide the title compound. ¹H NMR (400 MHz, dimethyl sulfoxide-d₆) δ ppm 12.85 (s, 1H), 9.99 (s, 1H), 8.06 (d, 1H), 8.03 (d, 1H), 7.79 (dd, 2H), 7.60 (dd, 3H), 7.55-7.41 (m, 3H), 7.36 (td, 2H), 7.29 (t, 3H), 6.99 (s, 2H), 6.95 (d, 1H), 5.99 (s, 1H), 5.04-4.92 (m, 4H), 4.37 (q, 1H), 4.34-4.24 (m, 1H), 4.24-4.10 (m, 4H), 3.88 (t, 2H), 3.82 (s, 2H), 3.40-3.29 (m, 4H), 3.01 (t, 2H), 2.99-2.91 (m, 1H), 2.87 (t, 2H), 2.25-2.06 (m, 5H), 1.95 (dt, 1H), 1.68 (s, 1H), 1.60 (s, 1H), 1.54-1.24 (m, 12H), 1.24-0.94 (m, 9H), 0.90-0.78 (m, 12H); MS (ESI) m/e 1507.4 (M+H)⁺.

### 1.90. Synthesis of 6-[8-(1,3-benzothiazol-2-ylcarbamoyl)-3,4-dihydroisoquinolin-2(1H)-yl]-3-{1-[(3-{[26-(2,5-dioxo-2,5-dihydro-1H-pyrrol-1-yl)-8,24-dioxo-3-(2-sulfoethyl)-11,14,17,20-tetraoxa-3,7,23-triazahexacos-1-yl]oxy}-5,7-dimethyltricyclo[3.3.1.1^{3,7}]dec-1-yl)methyl]-5-methyl-1H-pyrazol-4-yl}pyridine-2-carboxylic Acid (Synthon MS)

To a mixture of Example 1.61.2 (15 mg) and 2,5-dioxopyrrolidin-1-yl 1-(2,5-dioxo-2,5-dihydro-1H-pyrrol-1-yl)-3-oxo-7,10,13,16-tetraoxa-4-azanonadecan-19-oate (16.91 mg) in N,N-dimethylformamide (0.8 mL) was added N,N-diisopropylethylamine (28.8 μl) at 0° C. The mixture was stirred for 3 hours and purified by reverse phase HPLC, using a Gilson system and C18 column, eluting with 20-60% acetonitrile in water containing 0.1% trifluoroacetic acid, to provide the title compound. ¹H NMR (400 MHz, dimethyl sulfoxide-d₆) δ ppm 12.87 (s, 1H), 8.98 (s, 1H), 8.08-7.92 (m, 3H), 7.79 (d, 1H), 7.62 (d, 1H), 7.57-7.41 (m, 3H), 7.36 (td, 2H), 7.29 (s, 1H), 7.04-6.92 (m, 3H), 4.96 (s, 2H), 3.89 (t, 2H), 3.83 (s, 2H), 3.48 (d, 4H), 3.44-3.17 (m, 3H), 3.18-2.83 (m, 10H), 2.38-2.24 (m, 4H), 2.11 (s, 3H), 1.78 (m, 2H), 1.50-0.94 (m, 12H), 0.86 (s, 6H). MS (ESI) m/e 1309.3 (M−H).

### 1.91. Synthesis of N-[6-(2,5-dioxo-2,5-dihydro-1H-pyrrol-1-yl)hexanoyl]-L-valyl-N-[4-({[(3-{[2-({3-[(4-{6-[8-(1,3-benzothiazol-2-ylcarbamoyl)-3,4-dihydroisoquinolin-2(1H)-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](2-sulfoethyl)amino}propyl) carbamoyl]oxy}methyl)phenyl]-N⁵-carbamoyl-L-ornithinamide (Synthon MR)

To a mixture of Example 1.61.2 (12.8 mg) and 4-((S)-2-((S)-2-(6-(2,5-dioxo-2,5-dihydro-1H-pyrrol-1-yl) hexanamido)-3-methylbutanamido)-5-ureidopentanamido)benzyl (4-nitrophenyl) carbonate (10.4 mg) in N,N-dimethylformamide (0.5 mL) at 0° C. was added N,N-diisopropylethylamine (24.54 μl). The mixture was stirred for 3 hours and purified by reverse phase HPLC using a Gilson system and a C18 column, eluting with 20-60% acetonitrile in water containing 0.1% trifluoroacetic acid, to provide the title compound. ¹H NMR (400 MHz, dimethyl sulfoxide-d₆) δ ppm 12.85 (s, 1H), 9.97 (s, 1H), 8.97 (s, 1H), 8.04 (t, 2H), 7.79 (dd, 2H), 7.65-7.40 (m, 7H), 7.36 (td, 3H), 7.28 (d, 3H), 6.99 (s, 2H), 6.95 (d, 1H), 5.98 (s, 1H), 4.95 (d, 4H), 4.49-4.30 (m, 1H), 4.24-4.11 (m, 1H), 3.88 (t, 2H), 3.82 (s, 2H), 3.36 (t, 3H), 3.18-2.84 (m, 9H), 2.25-1.88 (m, 5H), 1.85-0.90 (m, 14H), 0.91-0.75 (m, 13H). MS (ESI) m/e (M+H).

### 1.92. Synthesis of N-{6-[(iodoacetyl)amino]hexanoyl}-L-valyl-N-{4-[({[2-({3-[(4-{6-[8-(1,3-benzothiazol-2-ylcarbamoyl)-3,4-dihydroisoquinolin-2(1H)-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](2-sulfoethyl)carbamoyl}oxy)methyl]phenyl}-N⁵-carbamoyl-L-ornithinamide (Synthon MQ)

To a mixture of Example 1.2.9 (8.2 mg) and 2,5-dioxopyrrolidin-1-yl 6-(2-iodoacetamido)hexanoate (4.7 mg) in N,N-dimethylformamide (0.3 mL) in an ice-bath was added N,N-diisopropylethylamine (3 μl). The mixture was stirred at 0° C. for 1.5 hours. The reaction was diluted with dimethyl sulfoxide, and the mixture purified by reverse phase HPLC using a Gilson system and a C18 column, eluting with 20-60% acetonitrile in water containing 0.1% trifluoroacetic acid, to provide the title compound. ¹H NMR (400 MHz, dimethyl sulfoxide-d₆) δ ppm 12.87 (s, 1H), 10.00 (s, 1H), 8.21 (d, 1H), 8.06 (dd, 2H), 7.81 (dd, 2H), 7.60 (t, 3H), 7.48 (ddd, 3H), 7.36 (td, 2H), 7.28 (d, 3H), 6.95 (d, 1H), 4.97 (d, 4H), 4.39 (q, 1H), 4.19 (t, 1H), 3.88 (t, 2H), 3.80 (d, 2H), 3.25 (d, 2H), 2.97 (dq, 6H), 2.63 (s, 2H), 2.25-1.88 (m, 5H), 1.78-0.70 (m, 29H). MS (ESI) m/e 1538.4 (M−H).

### 1.93. Synthesis of N-{6-[(ethenylsulfonyl)amino]hexanoyl}-L-valyl-N-{4-[({[2-({3-[(4-{6-[8-(1,3-benzothiazol-2-ylcarbamoyl)-3,4-dihydroisoquinolin-2(1H)-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](2-sulfoethyl)carbamoyl}oxy)methyl]phenyl}-N⁵-carbamoyl-L-ornithinamide (Synthon MZ)

### 1.93.1. Methyl 6-(vinylsulfonamido)hexanoate

To a solution of 6-methoxy-6-oxohexan-1-aminium chloride (0.3 g) and triethylamine (1.15 mL) in dichloromethane at 0° C. was added ethenesulfonyl chloride (0.209 g) dropwise. The reaction mixture was warmed to room temperature and stirred for 1 hour. The mixture was diluted with dichloromethane and washed with brine. The organic layer was dried over sodium sulfate, filtered, and concentrated to provide the title compound. MS (ESI) m/e 471.0 (2M+H)⁺.

### 1.93.2. 6-(vinylsulfonamido)hexanoic Acid

A solution of Example 2.93.1 (80 mg) and lithium hydroxide monohydrate (81 mg) in a mixture of tetrahydrofuran (1 mL) and water (1 mL) was stirred for 2 hours, then diluted with water (20 mL), and washed with diethyl ether (10 mL). The aqueous layer was acidified to pH 4 with 1N aqueous HCl and extracted with dichloromethane (3×10 mL). The organic layer was washed with brine (5 mL), dried over sodium sulfate, filtered and concentrated to provide the title compound.

### 1.93.3. 2,5-dioxopyrrolidin-1-yl 6-(vinylsulfonamido)hexanoate

A mixture of Example 2.93.2 (25 mg), 1-ethyl-3-[3-(dimethylamino)propyl]-carbodiimide hydrochloride (43.3 mg) and 1-hydroxypyrrolidine-2,5-dione (15.6 mg) in dichloromethane (8 mL) was stirred overnight, washed with saturated aqueous ammonium chloride solution and brine, and concentrated to provide the title compound.

### 1.93.4. N-{6-[(ethenylsulfonyl)amino]hexanoyl}-L-valyl-N-{4-[({[2-({3-[(4-{6-[8-(1,3-benzothiazol-2-ylcarbamoyl)-3,4-dihydroisoquinolin-2(1H)-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](2-sulfoethyl)carbamoyl}oxy)methyl]phenyl}-N⁵-carbamoyl-L-ornithinamide

The title compound was prepared as described in Example 2.83, replacing Example 1.2.9 and 2,5-dioxopyrrolidin-1-yl 6-(2-chloroacetamido)hexanoate with Example 2.66.1 and Example 2.93.3, respectively. ¹H NMR (400 MHz, dimethyl sulfoxide-d₆) δ ppm 12.85 (s, 1H), 9.98 (s, 1H), 8.05 (dd, 2H), 7.79 (d, 2H), 7.60 (t, 3H), 7.55-7.40 (m, 3H), 7.36 (td, 2H), 7.27 (d, 3H), 7.19 (t, 1H), 6.95 (d, 1H), 6.66 (dd, 1H), 6.09-5.90 (m, 2H), 4.97 (d, 4H), 4.39 (q, 1H), 4.20 (t, 1H), 3.88 (t, 2H), 3.80 (d, 2H), 3.25 (d, 2H), 2.97 (dt, 4H), 2.78 (q, 2H), 2.64 (q, 2H), 2.22-1.86 (m, 6H), 1.77-0.89 (m, 16H), 0.89-0.72 (m, 12H). MS (ESI) m/e 1460.6 (M−H)⁻.

### 1.94. Synthesis of 6-[8-(1,3-benzothiazol-2-ylcarbamoyl)-3,4-dihydroisoquinolin-2(1H)-yl]-3-(1-{[3-(2-{[3-({6-[(iodoacetyl)amino]hexanoyl}amino)propyl](2-sulfoethyl)amino}ethoxy)-5,7-dimethyltricyclo[3.3.1.1^{3,7}]dec-1-yl]methyl}-5-methyl-1H-pyrazol-4-yl)pyridine-2-carboxylic Acid (Synthon NA)

The title compound was prepared using the procedure in Example 2.83, replacing Example 1.2.9 and 2,5-dioxopyrrolidin-1-yl 6-(2-chloroacetamido)hexanoate with Example 2.61.2 and 2,5-dioxopyrrolidin-1-yl 6-(2-iodoacetamido)hexanoate, respectively. ¹H NMR (400 MHz, dimethyl sulfoxide-d₆) δ ppm 12.87 (s, 1H), 8.98 (s, 1H), 8.20 (t, 1H), 8.04 (d, 1H), 7.91 (t, 1H), 7.79 (d, 1H), 7.62 (d, 1H), 7.53 (d, 1H), 7.50-7.41 (m, 2H), 7.36 (td, 2H), 7.29 (s, 1H), 6.96 (d, 1H), 4.96 (s, 2H), 3.89 (t, 2H), 3.83 (s, 2H), 3.06 (dt, 8H), 2.89 (t, 2H), 2.17-1.99 (m, 5H), 1.76 (s, 2H), 1.56-0.93 (m, 14H), 0.86 (s, 6H). MS (ESI) m/e 1190.3 (M−H)⁻.

### 1.95. Synthesis of 6-[8-(1,3-benzothiazol-2-ylcarbamoyl)-3,4-dihydroisoquinolin-2(1H)-yl]-3-{1-[(3-{2-[(3-{[6-(ethenylsulfonyl)hexanoyl]amino}propyl)(2-sulfoethyl)amino]ethoxy}-5,7-dimethyltricyclo[3.3.1.1^{3,7}]dec-1-yl)methyl]-5-methyl-1H-pyrazol-4-yl}pyridine-2-carboxylic Acid (Synthon NB)

The title compound was prepared using the procedure in Example 2.83, replacing Example 1.2.9 and 2,5-dioxopyrrolidin-1-yl 6-(2-chloroacetamido)hexanoate with Example 1.61.2 and Example 2.82.5, respectively. ¹H NMR (400 MHz, dimethyl sulfoxide-d₆) δ ppm 12.87 (s, 1H), 8.98 (s, 1H), 8.04 (d, 1H), 7.92 (t, 1H), 7.79 (d, 1H), 7.62 (d, 1H), 7.53 (d, 1H), 7.51-7.41 (m, 2H), 7.36 (td, 2H), 7.29 (s, 1H), 7.01-6.90 (m, 2H), 6.29-6.16 (m, 2H), 4.96 (s, 2H), 3.89 (t, 2H), 3.83 (s, 2H), 3.45-3.19 (m, 2H), 3.19-2.95 (m, 8H), 2.89 (t, 2H), 2.16-1.98 (m, 5H), 1.84-1.66 (m, 2H), 1.64-1.21 (m, 13H), 1.08 (dq, 6H), 0.86 (s, 6H). MS (ESI) m/e 1199.3 (M+H).

### 1.96. Synthesis of N-[3-(2,5-dioxo-2,5-dihydro-1H-pyrrol-1-yl)propanoyl]-L-valyl-N-{4-[({[2-({3-[(4-{6-[1-(1,3-benzothiazol-2-ylcarbamoyl)-1,2,3,4-tetrahydroquinolin-7-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](2-sulfoethyl)carbamoyl}oxy)methyl]phenyl}-N⁵-carbamoyl-L-ornithinamide (Synthon NP)

### 1.96.1. (S)-(9H-fluoren-9-yl)methyl (1-((4-(hydroxymethyl)phenyl)amino)-1-oxo-5-ureidopentan-2-yl)carbamate

S)-2-((((9H-Fluoren-9-yl)methoxy)carbonyl)amino)-5-ureidopentanoic acid (40 g) was dissolved in dichloromethane (1.3 L). (4-Aminophenyl)methanol (13.01 g), 2-(3H-[1,2,3]triazolo[4,5-b]pyridin-3-yl)-1,1,3,3-tetramethylisouronium hexafluorophosphate(V) (42.1 g) and N,N-diisopropylethylamine (0.035 L) were added to the solution, and the resulting mixture was stirred at room temperature for 16 hours. The product was collected by filtration and rinsed with dichloromethane. The combined solids were dried under vacuum to yield the title compound, which was used in the next step without further purification. MS (ESI) m/e 503.3 (M+H)⁺.

### 1.96.2. (S)-2-amino-N-(4-(hydroxymethyl)phenyl)-5-ureidopentanamide

Example 2.96.1 (44 g) was dissolved in N,N-dimethylformamide (300 mL). The solution was treated with diethylamine (37.2 mL) and stirred for one hour at room temperature. The reaction mixture was filtered, and the solvent was concentrated under reduced pressure. The crude product was purified by basic alumina chromatography eluting with a gradient of 0-30% methanol in ethyl acetate to give the title compound. MS (ESI) m/e 281.2 (M+H)⁺.

### 1.96.3. tert-butyl ((S)-1-(((S)-1-((4-(hydroxymethyl)phenyl)amino)-1-oxo-5-ureidopentan-2-yl)amino)-3-methyl-1-oxobutan-2-yl)carbamate

(S)-2-(Tert-butoxycarbonylamino)-3-methylbutanoic acid (9.69 g) was dissolved in N,N-dimethylformamide (200 mL). To the solution was added 2-(3H-[1,2,3]triazolo[4,5-b]pyridin-3-yl)-1,1,3,3-tetramethylisouronium hexafluorophosphate(V) (18.65 g), and the reaction was stirred for one hour at room temperature. Example 2.96.2 (12.5 g) and N,N-diisopropylethylamine (15.58 mL) were added and the reaction mixture was stirred for 16 hours at room temperature. The solvent was concentrated under reduced pressure and the residue was purified by silica gel chromatography, eluting with 10% methanol in dichloromethane, to give the title compound. MS (ESI) m/e 480.2 (M+H)⁺.

### 1.96.4. (S)-2-((S)-2-amino-3-methylbutanamido)-N-(4-(hydroxymethyl)phenyl)-5-ureidopentanamide

Example 2.96.3 (31.8 g) was dissolved in dichloromethane (650 mL) and trifluoroacetic acid (4.85 mL) was added to the solution. The reaction mixture was stirred for three hours at room temperature. The solvent was concentrated under reduced pressure to yield a mixture of the crude title compound and 4-((S)-2-((S)-2-amino-3-methylbutanamido)-5-ureidopentanamido)benzyl 2,2,2-trifluoroacetate. The crude material was dissolved in a 1:1 dioxane/water solution (300 mL) and to the solution was added sodium hydroxide (5.55 g). The mixture was stirred for three hours at room temperature. The solvent was concentrated under vacuum, and the crude product was purified by reverse phase HPLC using a CombiFlash system, eluting with a gradient of 5-60% acetonitrile in water containing 0.05% v/v ammonium hydroxide, to give the title compound. MS (ESI) m/e 380.2 (M+H)⁺.

### 1.96.5. (S)-2-((S)-2-(3-(2,5-dioxo-2,5-dihydro-1H-pyrrol-1-yl)propanamido)-3-methylbutanamido)-N-(4-(hydroxymethyl)phenyl)-5-ureidopentanamide

To a solution of Example 2.96.4 (38 mg) in N,N-dimethylformamide (1 mL) was added 2,5-dioxopyrrolidin-1-yl 3-(2,5-dioxo-2,5-dihydro-1H-pyrrol-1-yl)propanoate (26.7 mg). The reaction mixture was stirred at room temperature overnight and purified by reverse phase HPLC using a Gilson system, eluting with a gradient of 10-85% acetonitrile in water containing 0.1% v/v trifluoroacetic acid, to give the title compound. MS (ESI) m/e 531.06 (M+H)⁺.

### 1.96.6. 4-((S)-2-((S)-2-(3-(2,5-dioxo-2,5-dihydro-1H-pyrrol-1-yl)propanamido)-3-methylbutanamido)-5-ureidopentanamido)benzyl (4-nitrophenyl) carbonate

To a solution of Example 2.96.5 (53.1 mg) in N,N-dimethylformamide (3 mL) was added bis(4-nitrophenyl) carbonate (60.8 mg). The reaction mixture was stirred at room temperature overnight and purified by reverse phase HPLC using a Gilson system, eluting with a gradient of 10-85% acetonitrile in water containing 0.1% v/v trifluoroacetic acid, to give the title compound. MS (ESI) m/e 696.2 (M+H)⁺.

### 1.96.7. N-[3-(2,5-dioxo-2,5-dihydro-1H-pyrrol-1-yl)propanoyl]-L-valyl-N-{4-[({[2-({3-[(4-{6-[1-(1,3-benzothiazol-2-ylcarbamoyl)-1,2,3,4-tetrahydroquinolin-7-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](2-sulfoethyl)carbamoyl}oxy)methyl]phenyl}-N⁵-carbamoyl-L-ornithinamide

The title compound was prepared as described in Example 2.1, replacing Example 1.2.9 and 4-((S)-2-((S)-2-(6-(2,5-dioxo-2,5-dihydro-1H-pyrrol-1-yl) hexanamido)-3-methylbutanamido)-5-ureidopentanamido)benzyl (4-nitrophenyl) carbonate with Example 1.24.2 and Example 2.96.6, respectively. ¹H NMR (400 MHz, dimethyl sulfoxide-d₆) δ ppm 9.91 (s, 1H), 9.80 (s, 2H), 8.33 (s, 2H), 7.96 (s, 2H), 7.81 (d, 4H), 7.61 (s, 2H), 7.43 (d, 10H), 7.34-7.02 (m, 14H), 5.92 (s, 8H), 4.94-4.70 (m, 6H), 4.18 (d, 11H), 3.85 (s, 8H), 3.05-2.66 (m, 8H), 2.30-2.13 (m, 14H), 2.03-1.49 (m, 2H), 0.92-0.63 (m, 40H). MS (ESI) m/e 1408.3 (M−H)⁺.

### 1.97. Synthesis of 6-[8-(1,3-benzothiazol-2-ylcarbamoyl)-3,4-dihydroisoquinolin-2(1H)-yl]-3-{1-[(3-{2-[(2-carboxyethyl) {[(2-{[(2S,3R,4S,5S,6S)-6-carboxy-3,4,5-trihydroxytetrahydro-2H-pyran-2-yl]oxy}-4-[2-(2-{[3-(2,5-dioxo-2,5-dihydro-1H-pyrrol-1-yl)propanoyl]amino}ethoxy)ethoxy]benzyl)oxy]carbonyl}amino]ethoxy}-5,7-dimethyltricyclo[3.3.1.1^{3,7}]dec-1-yl)methyl]-5-methyl-1H-pyrazol-4-yl}pyridine-2-carboxylic Acid (Synthon NN)

### 1.97.1. 4-(2-(2-bromoethoxy)ethoxy)-2-hydroxybenzaldehyde

A solution of 2,4-dihydroxybenzaldehyde (1.0 g), 1-bromo-2-(2-bromoethoxy) ethane (3.4 g) and potassium carbonate (1.0 g) in acetonitrile (30 mL) was heated to 75° C. for 2 days. The reaction was cooled, diluted with ethyl acetate (100 mL), washed with water (50 mL) and brine (50 mL), dried over magnesium sulfate, filtered and concentrated. Purification of the residue by silica gel chromatography, eluting with a gradient of 5-30% ethyl acetate in heptane, provided the title compound. MS (ELSD) m/e 290.4 (M+H)⁺.

### 1.97.2. 4-(2-(2-azidoethoxy)ethoxy)-2-hydroxybenzaldehyde

To a solution of Example 2.97.1 (1.26 g) in N,N-dimethylformamide (10 mL) was added sodium azide (0.43 g), and the reaction was stirred at room temperature overnight. The reaction was diluted with diethyl ether (100 mL), washed with water (50 mL) and brine (50 mL), dried over magnesium sulfate, filtered, and concentrated. Purification of the residue by silica gel chromatography, eluting with a gradient of 5-30% ethyl acetate in heptane, gave the title compound. MS (ELSD) m/e 251.4 (M+H)⁺.

### 1.97.3. (2S,3R,4S,5S,6S)-2-(5-(2-(2-azidoethoxy)ethoxy)-2-formylphenoxy)-6-(methoxycarbonyl)tetrahydro-2H-pyran-3,4,5-triyl Triacetate

A solution of Example 2.97.2 (0.84 g), (3R,4S,5S,6S)-2-bromo-6-(methoxycarbonyl)tetrahydro-2H-pyran-3,4,5-triyl triacetate (1.99 g) and silver (I) oxide (1.16 g) were stirred together in acetonitrile (15 mL). After stirring overnight, the reaction was diluted with dichloromethane (20 mL). Diatomaceous earth was added, and the reaction filtered and concentrated. Purification of the residue by silica gel chromatography, eluting with a gradient of 5-75% ethyl acetate in heptane, gave the title compound.

### 1.97.4. (2S,3R,4S,5S,6S)-2-(5-(2-(2-azidoethoxy)ethoxy)-2-(hydroxymethyl)phenoxy)-6-(methoxycarbonyl)tetrahydro-2H-pyran-3,4,5-triyl Triacetate

A solution of Example 2.97.3 (0.695 g) in methanol (5 mL) and tetrahydrofuran (2 mL) was cooled to 0° C. Sodium borohydride (0.023 g) was added, and the reaction was warmed to room temperature. After stirring for a total of 1 hour, the reaction was poured into a mixture of ethyl acetate (75 mL) and water (25 mL), and saturated aqueous sodium bicarbonate (10 mL) was added. The organic layer was separated, washed with brine (50 mL), dried over magnesium sulfate, filtered, and concentrated. Purification of the residue by silica gel chromatography, eluting with a gradient of 5-85% ethyl acetate in heptane, gave the title compound. MS (ELSD) m/e 551.8 (M−H₂O)⁻.

### 1.97.5. (2S,3R,4S,5S,6S)-2-(5-(2-(2-aminoethoxy)ethoxy)-2-(hydroxymethyl)phenoxy)-6-(methoxycarbonyl)tetrahydro-2H-pyran-3,4,5-triyl Triacetate

To Example 2.97.4 (0.465 g) in tetrahydrofuran (20 mL) was added 5% Pd/C (0.1 g) in a 50 mL pressure bottle, and the mixture was shaken for 16 hours under 30 psi hydrogen. The reaction was filtered and concentrated to give the title compound, which was used without further purification. MS (ELSD) m/e 544.1 (M+H)⁺.

### 1.97.6. (2S,3R,4S,5S,6S)-2-(5-(2-(2-((((9H-fluoren-9-yl)methoxy)carbonyl)amino)ethoxy)ethoxy)-2-(hydroxymethyl)phenoxy)-6-(methoxycarbonyl)tetrahydro-2H-pyran-3,4,5-triyl Triacetate

A solution of Example 2.97.5 (0.443 g) in dichloromethane (8 mL) was cooled to 0° C., then N,N-diisopropylethylamine (0.214 mL) and (9H-fluoren-9-yl)methyl carbonochloridate (0.190 g) were added. After 1 hour, the reaction was concentrated. Purification of the residue by silica gel chromatography, eluting with a gradient of 5-95% ethyl acetate in heptane, gave the title compound. MS (ELSD) m/e 748.15 (M-OH).

### 1.97.7. (2S,3R,4S,5S,6S)-2-(5-(2-(2-((((9H-fluoren-9-yl)methoxy)carbonyl)amino)ethoxy)ethoxy)-2-((((4-nitrophenoxy)carbonyl)oxy)methyl)phenoxy)-6-(methoxycarbonyl)tetrahydro-2H-pyran-3,4,5-triyl Triacetate

To a solution of Example 2.97.6 (0.444 g) in N,N-dimethylformamide (5 mL) was added N,N-diisopropylethylamine (0.152 mL) and bis(4-nitrophenyl) carbonate (0.353 g), and the reaction was stirred at room temperature. After 5 hours, the reaction was concentrated. Purification of the residue by silica gel chromatography, eluting with a gradient of 5-90% ethyl acetate in heptane, gave the title compound.

### 1.97.8. 3-(1-((3-(2-((((4-(2-(2-aminoethoxy)ethoxy)-2-(((2S,3R,4S,5S,6S)-6-carboxy-3,4,5-trihydroxytetrahydro-2H-pyran-2-yl)oxy)benzyl)oxy)carbonyl)(2-carboxyethyl)amino)ethoxy)-5,7-dimethyladamantan-1-yl)methyl)-5-methyl-1H-pyrazol-4-yl)-6-(8-(benzo[d]thiazol-2-ylcarbamoyl)-3,4-dihydroisoquinolin-2(1H)-yl)picolinic Acid, Trifluoroacetic Acid Salt

To a solution of Example 1.25 (0.070 g) and Example 2.97.7 (0.070 g) in N,N-dimethylformamide (0.4 mL) was added N,N-diisopropylethylamine (0.066 mL). After stirring overnight, the reaction was concentrated. The residue was dissolved in tetrahydrofuran (0.75 mL) and methanol (0.75 mL), and lithium hydroxide monohydrate (0.047 g) was added as a solution in water (0.75 mL). After 3 hours, the reaction was diluted with N,N-dimethylformamide (1 mL) and quenched with trifluoroacetic acid (0.116 mL). The mixture was purified by reverse phase HPLC using a Gilson system, eluting with 10-75% acetonitrile in water containing 0.1% v/v trifluoroacetic acid. The desired fractions were combined and freeze-dried to provide the title compound.

### 1.97.9. 6-(8-(benzo[d]thiazol-2-ylcarbamoyl)-3,4-dihydroisoquinolin-2(1H)-yl)-3-(1-((3-(2-((((2-(((2S,3R,4S,5S,6S)-6-carboxy-3,4,5-trihydroxytetrahydro-2H-pyran-2-yl)oxy)-4-(2-(2-(3-(2,5-dioxo-2,5-dihydro-1H-pyrrol-1-yl)propanamido)ethoxy)ethoxy)benzyl)oxy)carbonyl)(2-carboxyethyl)amino)ethoxy)-5,7-dimethyladamantan-1-yl)methyl)-5-methyl-1H-pyrazol-4-yl)picolinic acid

A solution of Example 2.97.8 (0.027 g), 2,5-dioxopyrrolidin-1-yl 3-(2,5-dioxo-2,5-dihydro-1H-pyrrol-1-yl)propanoate (7.92 mg) and N,N-diisopropylethylamine (0.017 mL) were stirred together in N,N-dimethylformamide (0.4 mL) for 1 hour. The reaction was quenched with a 1:1 mixture of water and N,N-dimethylformamide (2 mL). The mixture was purified by reverse phase HPLC using a Gilson system, eluting with 10-75% acetonitrile in water containing 0.1% v/v trifluoroacetic acid. The desired fractions were combined and freeze-dried to provide the title compound. ¹H NMR (400 MHz, dimethyl sulfoxide-d₆) δ ppm 12.81 (s, 1H), 8.03 (d, 2H), 7.79 (d, 1H), 7.62 (d, 1H), 7.54-7.40 (m, 3H), 7.36 (td, 2H), 7.28 (s, 1H), 7.18 (d, 1H), 6.98 (s, 2H), 6.95 (d, 1H), 6.69 (d, 1H), 6.60 (d, 1H), 5.03 (d, 3H), 4.96 (s, 2H), 4.05 (s, 2H), 3.93 (d, 2H), 3.88 (t, 2H), 3.80 (d, 2H), 3.75-3.67 (m, 2H), 3.59 (t, 6H), 3.29 (q, 6H), 3.17 (q, 2H), 3.01 (t, 2H), 2.47 (d, 2H), 2.33 (t, 2H), 2.09 (s, 3H), 1.44-0.88 (m, 12H), 0.82 (d, 6H); MS (ESI) m/e 1396.5 (M−H)⁻.

### 1.98. Synthesis of N-[6-(2,5-dioxo-2,5-dihydro-1H-pyrrol-1-yl)hexanoyl]-3-sulfo-L-alanyl-L-valyl-N-{4-[({[2-({3-[(4-{6-[8-(1,3-benzothiazol-2-ylcarbamoyl)-3,4-dihydroisoquinolin-2(1H)-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](2-carboxyethyl)carbamoyl}oxy)methyl]phenyl}-N⁵-carbamoyl-L-ornithinamide (Synthon NO)

### 1.98.1. 3-(1-((3-(2-((((4-((S)-2-((S)-2-amino-3-methylbutanamido)-5-ureidopentanamido)benzyl)oxy)carbonyl)(2-carboxyethyl)amino)ethoxy)-5,7-dimethyladamantan-1-yl)methyl)-5-methyl-1H-pyrazol-4-yl)-6-(8-(benzo[d]thiazol-2-ylcarbamoyl)-3,4-dihydroisoquinolin-2(1H)-yl)picolinic Acid

A solution of Example 1.25.2 (0.059 g), (9H-fluoren-9-yl)methyl ((S)-3-methyl-1-(((S)-1-((4-((((4-nitrophenoxy)carbonyl)oxy)methyl)phenyl)amino)-1-oxo-5-ureidopentan-2-yl)amino)-1-oxobutan-2-yl)carbamate (0.053 g) and N,N-diisopropylethylamine (0.055 mL) in N,N-dimethylformamide (0.5 mL) was stirred at room temperature overnight. Diethylamine (0.066 mL) was added to the reaction, and stirring was continued for 30 minutes. The reaction was diluted with a 1:1 mixture of N,N-dimethylformamide and water (2 mL) and quenched by the addition of trifluoroacetic acid (0.073 mL). The mixture was purified by reverse phase HPLC using a Gilson system, eluting with 10-75% acetonitrile in water containing 0.1% v/v trifluoroacetic acid. The desired fractions were combined and freeze-dried to provide the title compound. MS (ESI) m/e 1223.8 (M+H)⁺.

### 1.98.2. 3-(1-((3-(2-((((4-((S)-2-((S)-2-((R)-2-amino-3-sulfopropanamido)-3-methylbutanamido)-5-ureidopentanamido)benzyl)oxy)carbonyl)(2-carboxyethyl)amino)ethoxy)-5,7-dimethyladamantan-1-yl)methyl)-5-methyl-1H-pyrazol-4-yl)-6-(8-(benzo[d]thiazol-2-ylcarbamoyl)-3,4-dihydroisoquinolin-2(1H)-yl)picolinic Acid, Trifluoroacetic Acid Salt

A solution of (R)-2-((((9H-fluoren-9-yl)methoxy)carbonyl)amino)-3-sulfopropanoic acid (0.021 g), O-(7-azabenzotriazol-1-yl)-N,N,N′,N′-tetramethyluronium hexafluorophosphate (0.020 g) and N,N-diisopropylethylamine (0.031 mL) in N,N-dimethylformamide (0.4 mL) was stirred for 3 minutes. The solution was added to Example 2.98.1 (0.043 g) as a solution in N,N-dimethylformamide (0.4 mL). After stirring for 30 minutes, a solution of lithium hydroxide monohydrate (0.022 g) in water (0.5 mL) was added, and the reaction was stirred for 1 hour. The reaction was diluted with a 1:1 mixture of N,N-dimethylformamide and water (2 mL) and quenched by the addition of trifluoroacetic acid (0.054 mL). The mixture was purified by reverse phase HPLC using a Gilson system, eluting with 10-75% acetonitrile in water containing 0.1% v/v trifluoroacetic acid. The desired fractions were combined and freeze-dried to provide the title compound. MS (ESI) m/e 1376.5 (M+1).

### 1.98.3. 6-(8-(benzo[d]thiazol-2-ylcarbamoyl)-3,4-dihydroisoquinolin-2(1H)-yl)-3-(1-((3-(2-((2-carboxyethyl) (((4-((S)-2-((S)-2-((R)-2-(6-(2,5-dioxo-2,5-dihydro-1H-pyrrol-1-yl) hexanamido)-3-sulfopropanamido)-3-methylbutanamido)-5-ureidopentanamido)benzyl)oxy)carbonyl)amino)ethoxy)-5,7-dimethyladamantan-1-yl)methyl)-5-methyl-1H-pyrazol-4-yl)picolinic Acid

A solution of Example 2.98.2 (0.025 g), 2,5-dioxopyrrolidin-1-yl 6-(2,5-dioxo-2,5-dihydro-1H-pyrrol-1-yl)hexanoate (7.77 mg) and N,N-diisopropylethylamine (0.015 mL) in N,N-dimethylformamide (0.4 mL) was stirred for 1 hour. The reaction was diluted with a 1:1 mixture of water and N,N-dimethylformamide (2 mL). The mixture was purified by reverse phase HPLC using a Gilson system, eluting with 10-75% acetonitrile in water containing 0.1% v/v trifluoroacetic acid. The desired fractions were combined and freeze-dried to provide the title compound. ¹H NMR (400 MHz, dimethyl sulfoxide-d₆) δ ppm 12.85 (s, 1H), 9.46 (s, 1H), 8.20 (d, 1H), 8.07 (d, 1H), 8.03 (d, 1H), 8.00 (d, 1H), 7.79 (d, 1H), 7.69 (d, 2H), 7.61 (d, 1H), 7.51 (d, 1H), 7.49-7.45 (m, 1H), 7.43 (d, 1H), 7.36 (td, 2H), 7.29 (s, 1H), 7.25 (d, 2H), 6.97 (s, 2H), 6.95 (d, 1H), 4.98 (s, 2H), 4.96 (s, 2H), 4.73 (s, 2H), 4.16 (s, 2H), 4.03 (dd, 2H), 3.88 (t, 2H), 3.81 (s, 2H), 3.51-3.32 (m, 6H), 3.28 (t, 2H), 3.09 (dd, 1H), 3.06-2.94 (m, 4H), 2.89 (dd, 1H), 2.46 (d, 2H), 2.16 (dd, 1H), 2.09 (d, 4H), 1.74 (s, 2H), 1.62-1.29 (m, 8H), 1.29-0.92 (m, 12H), 0.92-0.78 (m, 12H). MS (ESI) m/e 1566.6 (M−H)⁻.

### 1.99. Synthesis of Control Synthon 4-[({[2-({3-[(4-{6-[8-(1,3-benzothiazol-2-ylcarbamoyl)-3,4-dihydroisoquinolin-2(1H)-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](methyl)carbamoyl}oxy)methyl]-2-({N-[6-(2,5-dioxo-2,5-dihydro-1H-pyrrol-1-yl)hexanoyl]-beta-alanyl}amino)phenyl Beta-D-Glucopyranosiduronic Acid (Synthon H)

### 1.99.1. (2S,3R,4S,5S,6S)-2-(4-formyl-2-nitrophenoxy)-6-(methoxycarbonyl)tetrahydro-2H-pyran-3,4,5-triyl Triacetate

To a solution of (2R,3R,4S,5S,6S)-2-bromo-6-(methoxycarbonyl)tetrahydro-2H-pyran-3,4,5-triyl triacetate (4 g) in acetonitrile (100 mL)) was added silver (I) oxide (10.04 g) and 4-hydroxy-3-nitrobenzaldehyde (1.683 g). The reaction mixture was stirred for 4 hours at room temperature and filtered. The filtrate was concentrated, and the residue was purified by silica gel chromatography, eluting with 5-50% ethyl acetate in heptanes, to provide the title compound. MS (ESI) m/e (M+18)⁺.

### 1.99.2. (2S,3R,4S,5S,6S)-2-(4-(hydroxymethyl)-2-nitrophenoxy)-6-(methoxycarbonyl)tetrahydro-2H-pyran-3,4,5-triyl Triacetate

To a solution of Example 2.99.1 (6 g) in a mixture of chloroform (75 mL) and isopropanol (18.75 mL) was added 0.87 g of silica gel. The resulting mixture was cooled to 0° C., NaBH₄ (0.470 g) was added, and the resulting suspension was stirred at 0° C. for 45 minutes. The reaction mixture was diluted with dichloromethane (100 mL) and filtered through diatomaceous earth. The filtrate was washed with water and brine and concentrated to give the crude product, which was used without further purification. MS (ESI) m/e (M+NH₄)⁺:

### 1.99.3. (2S,3R,4S,5S,6S)-2-(2-amino-4-(hydroxymethyl)phenoxy)-6-(methoxycarbonyl)tetrahydro-2H-pyran-3,4,5-triyl Triacetate

A stirred solution of Example 2.99.2 (7 g) in ethyl acetate (81 mL) was hydrogenated at 20° C. under 1 atmosphere H₂, using 10% Pd/C (1.535 g) as a catalyst for 12 hours. The reaction mixture was filtered through diatomaceous earth, and the solvent was evaporated under reduced pressure. The residue was purified by silica gel chromatography, eluting with 95/5 dichloromethane/methanol, to give the title compound.

### 1.99.4. 3-((((9H-fluoren-9-yl)methoxy)carbonyl)amino)propanoic Acid

3-Aminopropanoic acid (4.99 g) was dissolved in 10% aqueous Na₂CO₃ solution (120 mL) in a 500 mL flask and cooled with an ice bath. To the resulting solution, (9H-fluoren-9-yl)methyl carbonochloridate (14.5 g) in 1,4-dioxane (100 mL) was gradually added. The reaction mixture was stirred at room temperature for 4 hours, and water (800 mL) was then added. The aqueous phase layer was separated from the reaction mixture and washed with diethyl ether (3×750 mL). The aqueous layer was acidified with 2N HCl aqueous solution to a pH value of 2 and extracted with ethyl acetate (3×750 mL). The organic layers were combined and concentrated to obtain crude product. The crude product was recrystallized in a mixed solvent of ethyl acetate:hexane 1:2 (300 mL) to give the title compound.

### 1.99.5. (9H-fluoren-9-yl)methyl (3-chloro-3-oxopropyl)carbamate

To a solution of Example 2.99.4 in dichloromethane (160 mL) was added sulfurous dichloride (50 mL). The mixture was stirred at 60° C. for 1 hour. The mixture was cooled and concentrated to give the title compound.

### 1.99.6. (2S,3R,4S,5S,6S)-2-(2-(3-((((9H-fluoren-9-yl)methoxy)carbonyl)amino)propanamido)-4-(hydroxymethyl)phenoxy)-6-(methoxycarbonyl)tetrahydro-2H-pyran-3,4,5-triyl Triacetate

To a solution of Example 2.99.3 (6 g) in dichloromethane (480 mL) was added N,N-diisopropylethylamine (4.60 mL). Example 2.99.5 (5.34 g) was added, and the mixture was stirred at room temperature for 30 minutes. The mixture was poured into saturated aqueous sodium bicarbonate and was extracted with ethyl acetate. The combined extracts were washed with water and brine and were dried over sodium sulfate. Filtration and concentration gave a residue that was purified via radial chromatography, using 0-100% ethyl acetate in petroleum ether as mobile phase, to give the title compound.

### 1.99.7. (2S,3R,4S,5S,6S)-2-(2-(3-((((9H-fluoren-9-yl)methoxy)carbonyl)amino)propanamido)-4-((((4-nitrophenoxy)carbonyl)oxy)methyl)phenoxy)-6-(methoxycarbonyl)tetrahydro-2H-pyran-3,4,5-triyl Triacetate

To a mixture of Example 2.99.6 (5.1 g) in N,N-dimethylformamide (200 mL) was added bis(4-nitrophenyl) carbonate (4.14 g) and N,N-diisopropylethylamine (1.784 mL). The mixture was stirred for 16 hours at room temperature and concentrated under reduced pressure. The crude material was dissolved in dichloromethane and aspirated directly onto a 1 mm radial Chromatotron plate and eluted with 50-100% ethyl acetate in hexanes to give the title compound. MS (ESI) m/e (M+H)⁺.

### 1.99.8. 3-(1-((3-(2-((((3-(3-aminopropanamido)-4-(((2S,3R,4S,5S,6S)-6-carboxy-3,4,5-trihydroxytetrahydro-2H-pyran-2-yl)oxy)benzyl)oxy)carbonyl)(methyl)amino)ethoxy)-5,7-dimethyladamantan-1-yl)methyl)-5-methyl-1H-pyrazol-4-yl)-6-(8-(benzo[d]thiazol-2-ylcarbamoyl)-3,4-dihydroisoquinolin-2(1H)-yl)picolinic Acid

To a solution of Example 1.13.7 (325 mg) and Example 2.99.7 (382 mg) in N,N-dimethylformamide (9 mL) at 0° C. was added N,N-diisopropylamine (49.1 mg). The reaction mixture was stirred at 0° C. for 5 hours, and acetic acid (22.8 mg) was added. The resulting mixture was diluted with ethyl acetate and washed with water and brine. The organic layer was dried over Na₂SO₄, filtered and concentrated. The residue was dissolved in a mixture of tetrahydrofuran (10 mL) and methanol (5 mL). To this solution at 0° C. was added 1 M aqueous lithium hydroxide solution (3.8 mL). The resulting mixture was stirred at 0° C. for 1 hour, acidified with acetic acid and concentrated. The concentrate was lyophilized to provide a powder. The powder was dissolved in N,N-dimethylformamide (10 mL), cooled in an ice-bath, and piperidine (1 mL) at 0° C. was added. The mixture was stirred at 0° C. for 15 minutes and 1.5 mL of acetic acid was added. The solution was purified by reverse-phase HPLC using a Gilson system, eluting with 30-80% acetonitrile in water containing 0.1% v/v trifluoroacetic acid, to provide the title compound. MS (ESI) m/e 1172.2 (M+H)⁺.

### 1.99.9. 4-[({[2-({3-[(4-{6-[8-(1,3-benzothiazol-2-ylcarbamoyl)-3,4-dihydroisoquinolin-2(1H)-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](methyl)carbamoyl}oxy)methyl]-2-({N-[6-(2,5-dioxo-2,5-dihydro-1H-pyrrol-1-yl)hexanoyl]-beta-alanyl}amino)phenyl Beta-D-Glucopyranosiduronic Acid

To Example 2.99.8 (200 mg) in N,N-dimethylformamide (5 mL) at 0° C. was added 2,5-dioxopyrrolidin-1-yl 6-(2,5-dioxo-2,5-dihydro-1H-pyrrol-1-yl)hexanoate (105 mg) and N,N-diisopropylethylamine (0.12 mL). The mixture was stirred at 0° C. for 15 minutes, warmed to room temperature and purified by reverse-phase HPLC on a Gilson system using a 100 g C18 column, eluting with 30-80% acetonitrile in water containing 0.1% v/v trifluoroacetic acid, to provide the title compound. ¹H NMR (500 MHz, dimethyl sulfoxide-d₆) δ ppm 12.85 (s, 2H) 9.07 (s, 1H) 8.18 (s, 1H) 8.03 (d, 1H) 7.87 (t, 1H) 7.79 (d, 1H) 7.61 (d, 1H) 7.41-7.53 (m, 3H) 7.36 (q, 2H) 7.28 (s, 1H) 7.03-7.09 (m, 1H) 6.96-7.03 (m, 3H) 6.94 (d, 1H) 4.95 (s, 4H) 4.82 (t, 1H) 3.88 (t, 3H) 3.80 (d, 2H) 3.01 (t, 2H) 2.86 (d, 3H) 2.54 (t, 2H) 2.08 (s, 3H) 2.03 (t, 2H) 1.40-1.53 (m, 4H) 1.34 (d, 2H) 0.90-1.28 (m, 12H) 0.82 (d, 6H). MS (ESI) m/e 1365.3 (M+H)⁺.

### 1.100. Synthesis of Control Synthon 4-[({[2-({3-[(4-{6-[8-(1,3-benzothiazol-2-ylcarbamoyl)-3,4-dihydroisoquinolin-2(1H)-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](methyl)carbamoyl}oxy)methyl]-2-({N-[19-(2,5-dioxo-2,5-dihydro-1H-pyrrol-1-yl)-17-oxo-4,7,10,13-tetraoxa-16-azanonadecan-1-oyl]-beta-alanyl}amino)phenyl Beta-D-Glucopyranosiduronic Acid (Synthon I)

The title compound was prepared using the procedure in Example 2.99.9, replacing 2,5-dioxopyrrolidin-1-yl 6-(2,5-dioxo-2,5-dihydro-1H-pyrrol-1-yl)hexanoate with 2,5-dioxopyrrolidin-1-yl 1-(2,5-dioxo-2,5-dihydro-1H-pyrrol-1-yl)-3-oxo-7,10,13,16-tetraoxa-4-azanonadecan-19-oate. ¹H NMR (500 MHz, dimethyl sulfoxide-d₆) δ ppm 8.95 (s, 1H) 8.16 (s, 1H) 7.99 (d, 1H) 7.57-7.81 (m, 4H) 7.38-7.50 (m, 3H) 7.34 (q, 2H) 7.27 (s, 1H) 7.10 (d, 1H) 7.00 (d, 1H) 6.88-6.95 (m, 2H) 4.97 (d, 4H) 4.76 (d, 2H) 3.89 (t, 2H) 3.84 (d, 2H) 3.80 (s, 2H) 3.57-3.63 (m, 4H) 3.44-3.50 (m, 4H) 3.32-3.43 (m, 6H) 3.29 (t, 2H) 3.16 (q, 2H) 3.02 (t, 2H) 2.87 (s, 3H) 2.52-2.60 (m, 2H) 2.29-2.39 (m, 3H) 2.09 (s, 3H) 1.37 (s, 2H) 1.20-1.29 (m, 4H) 1.06-1.18 (m, 4H) 0.92-1.05 (m, 2H) 0.83 (s, 6H). MS (ESI) m/e 1568.6 (M−H)⁻.

### 2.101 Synthesis of 6-[8-(1,3-benzothiazol-2-ylcarbamoyl)-3,4-dihydroisoquinolin-2(1H)-yl]-3-{1-[(3-{[(43S,46S)-43-({[(4-{[(2S)-2-{[(2S)-2-{[6-(2,5-dioxo-2,5-dihydro-1H-pyrrol-1-yl)hexanoyl]amino}-3-methylbutanoyl]amino}propanoyl]amino}benzyl)oxy]carbonyl}amino)-46-methyl-37,44,47-trioxo-2,5,8,11,14,17,20,23,26,29,32,35-dodecaoxa-38,45,48-triazapentacontan-50-yl]oxy}-5,7-dimethyltricyclo[3.3.1.1^{3,7}]dec-1-yl)methyl]-5-methyl-1H-pyrazol-4-yl}pyridine-2-carboxylic Acid

The title compound was prepared as described in Example 2.7, replacing Example 1.13.8 with Example 1.66.7. ¹H NMR (400 MHz, dimethyl sulfoxide-d₆) δ ppm 12.85 (s, 1H), 8.21-7.97 (m, 4H), 7.79 (d, 4H), 7.71-7.32 (m, 15H), 7.28 (t, 4H), 7.02-6.91 (m, 3H), 4.95 (d, 5H), 4.33-4.12 (m, 3H), 3.98-3.76 (m, 11H), 3.41-3.21 (m, 22H), 3.21-2.90 (m, 12H), 2.24-2.05 (m, 7H), 1.81-0.90 (m, 46H), 0.90-0.78 (m, 17H). MS (ESI) m/e 2014.0 (M+H)⁺, 1007.5 (M+2H)²⁺, 672.0 (M+3H)³⁺.

### 2.102 Synthesis of N-[6-(2,5-dioxo-2,5-dihydro-1H-pyrrol-1-yl)hexanoyl]-L-valyl-N-{4-[({[2-({3-[(4-{6-[1-(1,3-benzothiazol-2-ylcarbamoyl)-1,2,3,4-tetrahydroquinolin-7-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](2-carboxyethyl)carbamoyl}oxy)methyl]phenyl}-N⁵-carbamoyl-L-ornithinamide

The title compound was prepared as described in Example 2.1, replacing Example 1.2.9 with Example 1.62.5 ¹H NMR (400 MHz, dimethyl sulfoxide-d₆) δ ppm 9.95 (s, 1H), 8.36 (s, 1H), 8.02 (d, 1H), 7.96 (d, 1H), 7.88-7.68 (m, 4H), 7.57 (d, 2H), 7.42 (s, 2H), 7.34 (t, 1H), 7.25 (dd, 3H), 7.19 (t, 1H), 6.95 (s, 2H), 5.96 (s, 1H), 4.96 (s, 2H), 4.35 (q, 1H), 4.15 (dd, 1H), 3.93 (t, 2H), 3.83 (d, 2H), 3.32 (t, 2H), 3.27 (d, 1H), 2.93 (dtd, 1H), 2.80 (t, 2H), 2.47 (p, 19H), 2.24-2.02 (m, 5H), 1.91 (p, 3H), 1.74-1.25 (m, 8H), 1.27-0.89 (m, 10H), 0.79 (dd, 13H). MS (ESI) m/e 1414.4 (M+H)⁺.

### 2.103 Synthesis of N-[6-(2,5-dioxo-2,5-dihydro-1H-pyrrol-1-yl)hexanoyl]-L-valyl-N-{4-[({[2-({3-[(4-{6-[8-(1,3-benzothiazol-2-ylcarbamoyl)naphthalen-2-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](2-carboxyethyl)carbamoyl}oxy)methyl]phenyl}-N⁵-carbamoyl-L-ornithinamide

The title compound was prepared as described in Example 2.1, replacing Example 1.2.9 with Example 1.68.7. ¹H NMR (400 MHz, dimethyl sulfoxide-d₆) δ ppm 13.07 (s, 1H), 9.95 (s, 1H), 8.99 (s, 1H), 8.33 (dd, 1H), 8.25-8.09 (m, 3H), 8.12-7.95 (m, 3H), 7.90 (d, 1H), 7.76 (dd, 2H), 7.73-7.63 (m, 1H), 7.56 (s, 3H), 7.41-7.29 (m, 1H), 6.95 (s, 2H), 5.97 (s, 1H), 4.96 (s, 2H), 4.35 (d, 2H), 4.15 (dd, 1H), 3.50-3.22 (m, 10H), 2.92 (dtd, 3H), 2.29-2.00 (m, 6H), 1.92 (q, 1H), 1.75-0.88 (m, 24H), 0.79 (dd, 15H). MS (ESI) m/e 1409.5 (M+H)⁺.

### 2.104 Synthesis of 6-[8-(1,3-benzothiazol-2-ylcarbamoyl)naphthalen-2-yl]-3-{1-[(3-{2-[(2-carboxyethyl) {[(2-{[(2R,3S,4R,5R,6R)-6-carboxy-3,4,5-trihydroxytetrahydro-2H-pyran-2-yl]oxy}-4-[2-(2-{[6-(2,5-dioxo-2,5-dihydro-1H-pyrrol-1-yl)hexanoyl]amino}ethoxy)ethoxy]benzyl)oxy]carbonyl}amino]ethoxy}-5,7-dimethyltricyclo[3.3.1.1^{3,7}]dec-1-yl)methyl]-5-methyl-1H-pyrazol-4-yl}pyridine-2-carboxylic Acid

### 2.104.1 3-(1-((3-(2-((((4-(2-(2-aminoethoxy)ethoxy)-2-(((2R,3S,4R,5R,6R)-6-carboxy-3,4,5-trihydroxytetrahydro-2H-pyran-2-yl)oxy)benzyl)oxy)carbonyl)(2-carboxyethyl)amino)ethoxy)-5,7-dimethyladamantan-1-yl)methyl)-5-methyl-1H-pyrazol-4-yl)-6-(8-(benzo[d]thiazol-2-ylcarbamoyl)naphthalen-2-yl)picolinic Acid

To a cold (0° C.) mixture of Example 2.97.7 (26.9 mg) and Example 1.68.7 (23.5 mg) in N,N-dimethylformamide (2 mL) was added N-ethyl-N-isopropylpropan-2-amine (0.043 mL). The reaction was slowly warmed to room temperature and stirred overnight. LC/MS showed the expected product as the major peak. To the reaction mixture was added water (1 mL) and LiOH H₂O (20 mg). The mixture was stirred at room temperature for 3 hours. The mixture was diluted with N,N-dimethylformamide (2 mL), filtered and purified by reverse-phase HPLC on a Gilson system (C18 column), eluting with 20-80% acetonitrile in water containing 0.1% trifluoroacetic acid, to give the title compound. MS (ESI) m/e 1242.2 (M−H)⁻.

### 2.104.2 6-[8-(1,3-benzothiazol-2-ylcarbamoyl)naphthalen-2-yl]-3-{1-[(3-{2-[(2-carboxyethyl) {[(2-{[(2R,3S,4R,5R,6R)-6-carboxy-3,4,5-trihydroxytetrahydro-2H-pyran-2-yl]oxy}-4-[2-(2-{[6-(2,5-dioxo-2,5-dihydro-1H-pyrrol-1-yl)hexanoyl]amino}ethoxy)ethoxy]benzyl)oxy]carbonyl}amino]ethoxy}-5,7-dimethyltricyclo[3.3.1.1^{3,7}]dec-1-yl)methyl]-5-methyl-1H-pyrazol-4-yl}pyridine-2-carboxylic Acid

The title compound was prepared as described in Example 2.97.9 by replacing Example 2.97.8 with Example 2.104.1 and replacing 2,5-dioxopyrrolidin-1-yl 3-(2,5-dioxo-2,5-dihydro-1H-pyrrol-1-yl)propanoate with 2,5-dioxopyrrolidin-1-yl 6-(2,5-dioxo-2,5-dihydro-1H-pyrrol-1-yl)hexanoate. ¹H NMR (400 MHz, dimethyl sulfoxide-d₆) δ ppm 13.06 (s, 2H), 8.99 (s, 1H), 8.34 (dd, 1H), 8.25-8.10 (m, 3H), 8.04 (d, 1H), 7.98 (d, 1H), 7.90 (d, 1H), 7.78 (d, 2H), 7.72-7.63 (m, 1H), 7.50-7.42 (m, 2H), 7.34 (t, 1H), 7.16 (d, 1H), 6.94 (s, 2H), 6.65 (d, 1H), 6.56 (dd, 1H), 4.02 (t, 2H), 3.90 (d, 1H), 3.83 (s, 2H), 3.66 (t, 3H), 3.28 (q, 4H), 3.15 (q, 2H), 2.19 (s, 3H), 1.99 (t, 2H), 1.51-1.30 (m, 6H), 1.28-0.88 (m, 11H), 0.81 (d, 6H). MS (ESI) m/e 1433.4 (M+H)⁺.

### 2.105 Synthesis of N-[6-(2,5-dioxo-2,5-dihydro-1H-pyrrol-1-yl)hexanoyl]-L-valyl-N-{4-[({[2-({3-[(4-{6-[5-(1,3-benzothiazol-2-ylcarbamoyl)quinolin-3-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](2-sulfoethyl)carbamoyl}oxy)methyl]phenyl}-N⁵-carbamoyl-L-ornithinamide

The title compound was prepared as described in Example 2.1, replacing Example 1.2.9 with Example 1.69.6. ¹H NMR (500 MHz, dimethyl sulfoxide-d₆) δ ppm 13.23 (s, 1H), 9.99 (s, 1H), 9.73 (d, 1H), 9.45 (s, 1H), 8.33 (t, 2H), 8.18 (d, 1H), 8.07 (dd, 2H), 8.02 (dd, 1H), 7.97 (dd, 1H), 7.80 (t, 2H), 7.65-7.55 (m, 2H), 7.53-7.44 (m, 2H), 7.37 (t, 1H), 7.27 (d, 2H), 6.98 (s, 2H), 4.98 (d, 2H), 4.38 (d, 1H), 4.18 (d, 1H), 3.56-3.31 (m, 3H), 3.26 (d, 2H), 3.08-2.89 (m, 2H), 2.64 (t, 2H), 2.23 (d, 3H), 2.12 (dp, 2H), 1.95 (s, 1H), 1.68 (s, 1H), 1.62-1.29 (m, 7H), 1.29-0.90 (m, 9H), 0.90-0.74 (m, 12H). MS (ESI) m/e 1446.3 (M−H)⁻.

### 2.106 Synthesis of N-[6-(2,5-dioxo-2,5-dihydro-1H-pyrrol-1-yl)hexanoyl]-L-valyl-N-{4-[({[2-({3-[(4-{6-[4-(1,3-benzothiazol-2-ylcarbamoyl)quinolin-6-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](2-carboxyethyl)carbamoyl}oxy)methyl]phenyl}-N⁵-carbamoyl-L-ornithinamide

The title compound was prepared as described in Example 2.1, replacing Example 1.2.9 with Example 1.70. ¹H NMR (400 MHz, dimethyl sulfoxide-d₆) δ ppm 9.97 (s, 1H), 9.12 (d, 1H), 8.93 (s, 1H), 8.60 (dd, 1H), 8.24 (dd, 2H), 8.05 (dd, 2H), 7.99-7.87 (m, 2H), 7.78 (dd, 2H), 7.67-7.51 (m, 3H), 7.43-7.31 (m, 1H), 7.26 (d, 2H), 6.97 (s, 2H), 5.98 (s, 1H), 4.97 (s, 2H), 4.37 (d, 2H), 4.17 (dd, 1H), 3.49-3.22 (m, 11H), 2.95 (ddd, 3H), 2.20 (s, 4H), 2.19-1.86 (m, 3H), 1.74-0.89 (m, 22H), 0.81 (dd, 15H). MS (ESI) m/e 1410.4 (M−H)⁻.

### 2.107 Synthesis of N-[6-(2,5-dioxo-2,5-dihydro-1H-pyrrol-1-yl)hexanoyl]-L-valyl-N-{4-[({[2-({3-[(4-{6-[4-(1,3-benzothiazol-2-ylcarbamoyl)quinolin-6-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](2-sulfoethyl)carbamoyl}oxy)methyl]phenyl}-N⁵-carbamoyl-L-ornithinamide

The title compound was prepared as described in Example 2.1, replacing Example 1.2.9 with Example 1.70.5. ¹H NMR (400 MHz, dimethyl sulfoxide-d₆) δ ppm 9.96 (s, 1H), 9.11 (d, 1H), 8.92 (s, 1H), 8.60 (dd, 1H), 8.23 (dd, 2H), 8.12-7.97 (m, 2H), 7.98-7.92 (m, 2H), 7.77 (dd, 2H), 7.56 (t, 2H), 7.51-7.42 (m, 2H), 7.42-7.31 (m, 1H), 7.24 (d, 2H), 6.95 (s, 2H), 4.95 (d, 2H), 4.36 (q, 1H), 3.90-3.80 (m, 3H), 3.52-3.27 (m, 3H), 3.23 (t, 2H), 3.06-2.83 (m, 2H), 2.67-2.58 (m, 2H), 2.19 (s, 3H), 2.09 (dp, 2H), 1.93 (d, 1H), 1.72-1.25 (m, 7H), 1.27-0.88 (m, 10H), 0.88-0.70 (m, 13H). MS (ESI) m/e 1446.3 (M−H)⁻.

### 2.108 Synthesis of N-[6-(2,5-dioxo-2,5-dihydro-1H-pyrrol-1-yl)hexanoyl]-L-valyl-N-{4-[({[2-({3-[(4-{6-[5-(1,3-benzothiazol-2-ylcarbamoyl)quinolin-3-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](2-carboxyethyl)carbamoyl}oxy)methyl]phenyl}-N⁵-carbamoyl-L-ornithinamide

The title compound was prepared as described in Example 2.1, replacing Example 1.2.9 with Example 1.71. ¹H NMR (400 MHz, dimethyl sulfoxide-d₆) δ ppm 9.97 (s, 1H), 9.70 (d, 1H), 9.40 (d, 1H), 8.31 (dd, 2H), 8.16 (d, 1H), 8.05 (t, 2H), 8.01-7.91 (m, 2H), 7.78 (dd, 2H), 7.59 (d, 3H), 7.52-7.44 (m, 2H), 7.36 (t, 1H), 7.26 (d, 2H), 6.96 (s, 2H), 5.99 (s, 1H), 4.97 (s, 2H), 4.37 (d, 2H), 4.16 (dd, 1H), 3.53-3.20 (m, 9H), 2.94 (dtd, 2H), 2.21 (s, 3H), 2.17-1.85 (m, 3H), 1.71-0.89 (m, 22H), 0.81 (dd, 14H). MS (ESI) m/e 1410.4 (M−H)⁻.

### 2.109 Synthesis of N-[6-(2,5-dioxo-2,5-dihydro-1H-pyrrol-1-yl)hexanoyl]-L-valyl-N-{4-[({[2-({3-[(4-{6-[1-(1,3-benzothiazol-2-ylcarbamoyl)-5,6-dihydroimidazo[1,5-a]pyrazin-7 (8H)-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](2-sulfoethyl)carbamoyl}oxy)methyl]phenyl}-N⁵-carbamoyl-L-ornithinamide

The title compound was prepared by substituting Example 1.72.8 for Example 1.2.9 in Example 2.1. ¹H NMR (400 MHz, dimethyl sulfoxide d₆) δ ppm 11.07 (bs, 1H), 10.00 (bs, 1H), 8.27 (bs, 1H), 8.12 (m, 2H), 8.07 (d, 1H), 7.99 (d, 1H), 7.84-7.74 (m, 2H), 7.65 (d, 1H), 7.59 (m, 2H), 7.54-7.44 (m, 1H), 7.42-7.31 (m, 2H), 7.28 (m, 2H), 7.21 (q, 1H), 7.00 (m, 1H) 6.94-6.92 (m, 2H), 6.04 (bs, 1H), 5.14 (s, 2H), 4.99 (m, 3H), 4.39 (m, 2H), 4.30 (bs, 2H), 4.20 (m, 2H), 4.12 (bs, 2H), 3.70-3.64 (m, 2H), 3.50 (m, 2H), 3.44-3.35 (m, 2H), 3.27 (m, 2H), 3.02 (m, 2H), 2.95 (m, 2H), 2.68 (t, 2H), 2.14 (m, 4H), 1.96 (m, 1H), 1.69 (m, 1H), 1.58 (m, 1H), 1.47 (m, 4H), 1.36 (m, 2H), 1.30-1.02 (m, 8H), 0.98 (m, 2H), 0.85-0.80 (m, 16H).

### 2.110 Synthesis of N-[6-(2,5-dioxo-2,5-dihydro-1H-pyrrol-1-yl)hexanoyl]-L-valyl-N-{4-[({[2-({3-[(4-{6-[7-(1,3-benzothiazol-2-ylcarbamoyl)-1H-indol-2-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](2-sulfoethyl)carbamoyl}oxy)methyl]phenyl}-N⁵-carbamoyl-L-ornithinamide

Example 2.110 was prepared by substituting Example 1.74.6 for Example 1.2.9 in Example 2.1. ¹H NMR (400 MHz, dimethyl sulfoxide-d₆) δ ppm 11.30 (s, 1H), 9.93 (s, 1H), 8.26 (d, 1H), 8.17 (d, 1H), 8.02 (d, 1H), 7.92-7.84 (m, 3H), 7.76 (d, 1H), 7.69 (d, 1H), 7.54 (d, 3H), 7.47 (s, 1H), 7.35 (dd, 2H), 7.22 (t, 3H), 7.08 (t, 1H), 6.93 (s, 2H), 4.90 (s, 2H), 4.84 (t, 2H), 4.33 (q, 1H), 4.16-4.09 (m, 1H), 3.32 (t, 4H), 2.99 (m, 6H), 2.21 (s, 3H), 2.09 (m, 2H), 1.91 (m, 1H), 1.71-0.71 (m, 25H). MS (ESI) m/e 1434.4 (M−H)⁻.

### 2.111 Synthesis of N-[6-(2,5-dioxo-2,5-dihydro-1H-pyrrol-1-yl)hexanoyl]-L-valyl-N-[4-({[{3-[8-(1,3-benzothiazol-2-ylcarbamoyl)-2-(6-carboxy-5-{1-[(3,5-dimethyl-7-{2-[(2-sulfoethyl)amino]ethoxy}tricyclo[3.3.1.1^{3,7}]dec-1-yl)methyl]-5-methyl-1H-pyrazol-4-yl}pyridin-2-yl)-1,2,3,4-tetrahydroisoquinolin-6-yl]propyl}(methyl)carbamoyl]oxy}methyl)phenyl]-N⁵-carbamoyl-L-ornithinamide

The title compound was prepared as described in Example 2.1, replacing Example 1.2.9 with Example 1.75.14. ¹H NMR (400 MHz, dimethyl sulfoxide-d₆) δ ppm 12.60 (bs, 1H), 9.98 (s, 1H), 8.33 (m, 2H), 8.02 (d, 2H), 7.75 (d, 2H), 7.55 (d, 2H), 7.49 (m, 3H), 7.29 (m, 1H), 7.25 (s, 4H), 6.99 (d, 2H), 6.95 (d, 1H), 5.90 (m, 1H), 5.42 (m, 2H), 4.95 (s, 2H), 4.90 (m, 2H), 4.35 (t, 1H), 4.18 (t, 1H), 3.85 (m, 2H), 3.80 (s, 3H), 3.55 (s, 3H), 3.52 (m, 2H), 3.35 (m, 4H), 3.22 (m, 4H), 3.08 (m, 2H), 2.99 (m, 2H), 2.92 (m, 2H), 2.85 (m, 2H), 2.79 (t, 2H), 2.52 (m, 1H), 2.15 (m, 1H), 2.09 (s, 3H), 1.94 (m, 1H), 1.88 (m, 1H), 1.68 (m, 1H), 1.54 (m, 1H), 1.42 (m, 4H), 1.38 (m, 4H), 1.27 (m, 4H), 1.13 (m, 4H), 1.02 (m, 2H), 0.85 (s, 6H), 0.78 (m, 6H). MS (ESI) m/e 1523.3 (M+H)⁺, 1521.6 (M−H)⁻.

### 2.112 Synthesis of N-(6-{[(2,5-dioxo-2,5-dihydro-1H-pyrrol-1-yl)acetyl]amino}hexanoyl)-L-valyl-N-{4-[({[2-({3-[(4-{6-[8-(1,3-benzothiazol-2-ylcarbamoyl)-3,4-dihydroisoquinolin-2(1H)-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](2-sulfoethyl)carbamoyl}oxy)methyl]phenyl}-N⁵-carbamoyl-L-ornithinamide

### 2.112.1 6-(8-(benzo[d]thiazol-2-ylcarbamoyl)-3,4-dihydroisoquinolin-2(1H)-yl)-3-(1-((3-(2-((((4-((S)-2-((S)-2-((tert-butoxycarbonyl)amino)-3-methylbutanamido)-5-ureidopentanamido)benzyl)oxy)carbonyl)(2-sulfoethyl)amino)ethoxy)-5,7-dimethyladamantan-1-yl)methyl)-5-methyl-1H-pyrazol-4-yl)picolinic Acid

Example 1.2.9, trifluoroacetic acid salt (390 mg), tert-butyl ((S)-3-methyl-1-(((S)-1-((4-((((4-nitrophenoxy)carbonyl)oxy)methyl)phenyl)amino)-1-oxo-5-ureidopentan-2-yl)amino)-1-oxobutan-2-yl)carbamate (286 mg) and 1-hydroxybenzotriazole hydrate (185 mg) in N,N-dimethylformamide (5 mL) was cooled in an ice-bath and N,N-diisopropylethylamine (0.35 mL) was added. The mixture was stirred at 0° C. for 30 minutes and at room temperature overnight. The reaction mixture was diluted with dimethyl sulfoxide to 10 mL and purified by reverse-phase HPLC on a Gilson system (C18 column), eluting with 20-80% acetonitrile in water containing 0.1% trifluoroacetic acid, to give the title compound. MS (ESI) m/e 680.1 (M+2H)²⁺.

### 2.112.2 3-(1-((3-(2-((((4-((S)-2-((S)-2-amino-3-methylbutanamido)-5-ureidopentanamido)benzyl)oxy)carbonyl)(2-sulfoethyl)amino)ethoxy)-5,7-dimethyladamantan-1-yl)methyl)-5-methyl-1H-pyrazol-4-yl)-6-(8-(benzo[d]thiazol-2-ylcarbamoyl)-3,4-dihydroisoquinolin-2(1H)-yl)picolinic Acid

Example 2.112.1 (300 mg) in 10 mL of dichloromethane at 0° C. was treated with trifluoroacetic acid (4 mL) for 30 minutes and the mixture was concentrated. The residue was dissolved in a mixture of acetonitrile and water and lyophilized to provide the desired product as a TFA salt. MS (ESI) m/e 1257.4 (M−H)⁻.

### 2.112.3 6-(8-(benzo[d]thiazol-2-ylcarbamoyl)-3,4-dihydroisoquinolin-2(1H)-yl)-3-(1-((3-(2-((((4-((13S,16S)-13-isopropyl-2,2-dimethyl-4,11,14-trioxo-16-(3-ureidopropyl)-3-oxa-5,12,15-triazaheptadecanamido)benzyl)oxy)carbonyl)(2-sulfoethyl)amino)ethoxy)-5,7-dimethyladamantan-1-yl)methyl)-5-methyl-1H-pyrazol-4-yl)picolinic Acid

Example 2.112.2 (trifluoroacetic acid salt, 385 mg,) and 1-hydroxybenzotriazole hydrate (140 mg) in N,N-dimethylformamide (3 mL) was cooled in an ice-water bath. N,N-Diisopropylethylamine (226 μL) was added dropwise, followed by the addition of 2,5-dioxopyrrolidin-1-yl 6-((tert-butoxycarbonyl)amino)hexanoate (127 mg), and the mixture was stirred overnight. The mixture was purified by reverse-phase HPLC on a Gilson system (C18 column), eluting with 20-75% acetonitrile in water containing 0.1% trifluoroacetic acid, to give the title compound. MS (ESI) m/e 1470.2 (M−H)⁻.

### 2.112.4 3-(1-((3-(2-((((4-((S)-2-((S)-2-(6-aminohexanamido)-3-methylbutanamido)-5-ureidopentanamido)benzyl)oxy)carbonyl)(2-sulfoethyl)amino)ethoxy)-5,7-dimethyladamantan-1-yl)methyl)-5-methyl-1H-pyrazol-4-yl)-6-(8-(benzo[d]thiazol-2-ylcarbamoyl)-3,4-dihydroisoquinolin-2(1H)-yl)picolinic Acid

The title compound was prepared using the procedure in Example 2.112.2, replacing Example 2.112.1 with Example 2.112.3. MS (ESI) m/e 1370.5 (M−H)⁻.

### 2.112.5 N-(6-{[(2,5-dioxo-2,5-dihydro-1H-pyrrol-1-yl)acetyl]amino}hexanoyl)-L-valyl-N-{4-[({[2-({3-[(4-{6-[8-(1,3-benzothiazol-2-ylcarbamoyl)-3,4-dihydroisoquinolin-2(1H)-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](2-sulfoethyl)carbamoyl}oxy)methyl]phenyl}-N⁵-carbamoyl-L-ornithinamide

Example 2.112.4 (25 mg) and 2,5-dioxopyrrolidin-1-yl 2-(2,5-dioxo-2,5-dihydro-1H-pyrrol-1-yl)acetate (9.19 mg) in N,N-dimethylformamide (0.3 mL) was treated with N,N-diisopropylethylamine (25.4 μL) for 30 minutes at 0° C. The reaction mixture was purified by reverse-phase HPLC on a Gilson system (C18 column), eluting with 35-65% acetonitrile in 4 mM ammonium acetate water mixture, to provide the title compound as an ammonium salt. ¹H NMR (400 MHz, dimethyl sulfoxide-d₆) δ 12.81 (s, 1H), 9.94 (s, 1H), 8.01 (dd, 2H), 7.75 (d, 2H), 7.56 (s, 3H), 7.51-7.45 (m, 1H), 7.45-7.37 (m, 2H), 7.36-7.28 (m, 2H), 7.24 (t, 3H), 7.17 (s, 2H), 7.05 (s, 3H), 7.04 (s, 2H), 6.92 (s, 3H), 5.93 (s, 1H), 5.36 (s, 2H), 5.05-4.85 (m, 4H), 4.36 (q, 1H), 4.16 (dd, 1H), 3.95 (s, 2H), 3.85 (t, 2H), 3.76 (d, 2H), 3.22 (d, 1H), 3.05-2.81 (m, 6H), 2.68-2.53 (m, 2H), 2.09 (d, 4H), 1.76-0.86 (m, 14H), 0.86-0.71 (m, 12H). MS (ESI) m/e 1507.5 (M−H)⁻.

### 2.113 Synthesis of N-[6-(2,5-dioxo-2,5-dihydro-1H-pyrrol-1-yl)hexanoyl]-L-valyl-N-{4-[({[2-({3-[(4-{6-[8-(1,3-benzothiazol-2-ylcarbamoyl)naphthalen-2-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl][3-(beta-L-glucopyranuronosyloxy)propyl]carbamoyl}oxy)methyl]phenyl}-N⁵-carbamoyl-L-ornithinamide

The title compound was prepared as described in Example 2.1, replacing Example 1.2.9 with Example 1.87.3. ¹H NMR (501 MHz, dimethyl sulfoxide-d₆) δ ppm 13.08 (s, 1H), 9.96 (s, 1H), 9.00 (s, 1H), 8.35 (dd, 1H), 8.24-8.13 (m, 3H), 8.09-8.02 (m, 2H), 8.00 (d, 1H), 7.91 (d, 1H), 7.77 (dd, 2H), 7.71-7.64 (m, 1H), 7.58 (t, 2H), 7.49-7.44 (m, 2H), 7.39-7.32 (m, 1H), 7.26 (d, 2H), 6.96 (s, 2H), 5.97 (s, 1H), 4.96 (s, 2H), 4.37 (d, 1H), 4.22-4.12 (m, 2H), 3.84 (s, 1H), 3.37-3.20 (m, 6H), 3.15 (t, 1H), 3.04-2.81 (m, 2H), 2.20 (s, 3H), 2.11 (dp, 2H), 1.99-1.88 (m, 1H), 1.71 (q, 2H), 1.62-1.26 (m, 8H), 1.29-0.88 (m, 11H), 0.80 (dd, 14H). MS (ESI) m/e 1571.4 (M−H)⁻.

### 2.114 Synthesis of N-[6-(2,5-dioxo-2,5-dihydro-1H-pyrrol-1-yl)hexanoyl]-L-valyl-N-{4-[({[2-({3-[(4-{6-[4-(1,3-benzothiazol-2-ylcarbamoyl) isoquinolin-6-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](2-sulfoethyl)carbamoyl}oxy)methyl]phenyl}-N⁵-carbamoyl-L-ornithinamide

The title compound was prepared as described in Example 2.1, replacing Example 1.2.9 with Example 1.78.5. ¹H NMR (400 MHz, dimethyl sulfoxide-d₆) δ ppm 9.95 (s, 1H), 9.61 (s, 1H), 9.08 (s, 1H), 9.00 (s, 1H), 8.54 (dd, 1H), 8.43 (d, 1H), 8.24 (d, 1H), 8.08-7.95 (m, 3H), 7.77 (dd, 2H), 7.63-7.51 (m, 2H), 7.50-7.42 (m, 2H), 7.40-7.31 (m, 1H), 7.24 (d, 2H), 6.95 (s, 2H), 6.00 (s, 1H), 4.95 (d, 2H), 4.36 (q, 1H), 4.15 (t, 1H), 3.27 (dt, 4H), 3.10-2.79 (m, 2H), 2.68-2.56 (m, 2H), 2.20 (s, 3H), 1.98-1.84 (m, 1H), 1.72-0.87 (m, 19H), 0.79 (dd, 13H). MS (ESI) m/e 1446.4 (M−H)⁻.

### 2.115 Synthesis of N-[6-(2,5-dioxo-2,5-dihydro-1H-pyrrol-1-yl)hexanoyl]-L-alpha-glutamyl-L-valyl-N-{4-[({[2-({3-[(4-{6-[8-(1,3-benzothiazol-2-ylcarbamoyl)-3,4-dihydroisoquinolin-2(1H)-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](2-sulfoethyl)carbamoyl}oxy)methyl]phenyl}-N⁵-carbamoyl-L-ornithinamide

### 2.115.1 6-(8-(benzo[d]thiazol-2-ylcarbamoyl)-3,4-dihydroisoquinolin-2(1H)-yl)-3-(1-((3-(2-((((4-((6S,9S,12S)-6-(3-(tert-butoxy)-3-oxopropyl)-9-isopropyl-2,2-dimethyl-4,7,10-trioxo-12-(3-ureidopropyl)-3-oxa-5,8,11-triazatridecanamido)benzyl)oxy)carbonyl)(2-sulfoethyl)amino)ethoxy)-5,7-dimethyladamantan-1-yl)methyl)-5-methyl-1H-pyrazol-4-yl)picolinic Acid

To a mixture of Example 2.112.2 (85 mg), 1-hydroxybenzotriazole hydrate (41.3 mg), and (S)-5-tert-butyl 1-(2,5-dioxopyrrolidin-1-yl) 2-((tert-butoxycarbonyl)amino)pentanedioate (54.0 mg) in N,N-dimethylformamide (3 mL) at 0° C. was added N,N-diisopropylethylamine (118 μL) dropwise, and the mixture was stirred at 0° C. for 1 hour. The mixture was purified by reverse-phase HPLC on a Gilson system (C18 column), eluting with 35-100% acetonitrile in water containing 0.1% trifluoroacetic acid, to give the title compound. MS (ESI) m/e 773.4 (M+2H)²⁺.

### 2.115.2 3-(1-((3-(2-((((4-((S)-2-((S)-2-((S)-2-amino-4-carboxybutanamido)-3-methylbutanamido)-5-ureidopentanamido)benzyl)oxy)carbonyl)(2-sulfoethyl)amino)ethoxy)-5,7-dimethyladamantan-1-yl)methyl)-5-methyl-1H-pyrazol-4-yl)-6-(8-(benzo[d]thiazol-2-ylcarbamoyl)-3,4-dihydroisoquinolin-2(1H)-yl)picolinic Acid

Example 2.115.1 (100 mg) in dichloromethane (11 mL) at 0° C. was treated with trifluoroacetic acid (4 mL). The mixture was stirred at 0° C. for 3.5 hours and concentrated. The residue was purified by reverse phase HPLC, eluting with 5-60% acetonitrile in 0.1% trifluoroacetic acid water mixture to provide the title compound.

### 2.115.3 N-[6-(2,5-dioxo-2,5-dihydro-1H-pyrrol-1-yl)hexanoyl]-L-alpha-glutamyl-L-valyl-N-{4-[({[2-({3-[(4-{6-[8-(1,3-benzothiazol-2-ylcarbamoyl)-3,4-dihydroisoquinolin-2(1H)-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](2-sulfoethyl)carbamoyl}oxy)methyl]phenyl}-N⁵-carbamoyl-L-ornithinamide

To a mixture of 1-hydroxybenzotriazole hydrate (2.87 mg), 2,5-dioxopyrrolidin-1-yl 6-(2,5-dioxo-2,5-dihydro-1H-pyrrol-1-yl)hexanoate (5.77 mg) and Example 2.115.2 (13 mg) at 0° C. was added N,N-diisopropylethylamine (13.08 μL), and the mixture was stirred at 0° C. for 1 hour. The reaction was purified by reverse-phase HPLC on a Gilson system (C18 column), eluting with 20-75% acetonitrile in water containing 0.1% trifluoroacetic acid, to give the title compound. ¹H NMR (501 MHz, dimethyl sulfoxide-d₆) δ 12.83 (s, 1H), 9.99 (s, 1H), 8.13 (d, 1H), 8.02 (dd, 1H), 7.97 (d, 1H), 7.80-7.74 (m, 1H), 7.64 (t, 1H), 7.61-7.48 (m, 4H), 7.47-7.38 (m, 2H), 7.38-7.30 (m, 2H), 7.29-7.23 (m, 3H), 6.96 (s, 2H), 6.93 (d, 1H), 5.99 (s, 1H), 5.06-4.88 (m, 5H), 4.37 (q, 1H), 4.28 (q, 1H), 4.18 (dd, 1H), 3.86 (t, 2H), 3.78 (d, 2H), 3.34 (t, 3H), 3.23 (d, 2H), 2.99 (t, 3H), 2.97-2.85 (m, 1H), 2.62 (dt, 1H), 2.26-2.15 (m, 2H), 2.16-2.00 (m, 5H), 2.01-1.79 (m, 1H), 1.75-1.50 (m, 3H), 1.50-0.87 (m, 17H), 0.81 (dd, 14H). MS (ESI) m/e 1579.6 (M−H)⁻.

### 2.116 Synthesis of N-[(2,5-dioxo-2,5-dihydro-1H-pyrrol-1-yl)acetyl]-L-alpha-glutamyl-L-valyl-N-{4-[({[2-({3-[(4-{6-[8-(1,3-benzothiazol-2-ylcarbamoyl)-3,4-dihydroisoquinolin-2(1H)-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](2-sulfoethyl)carbamoyl}oxy)methyl]phenyl}-N⁵-carbamoyl-L-ornithinamide

The title compound was prepared using the procedure in Example 2.115.3, replacing 2,5-dioxopyrrolidin-1-yl 6-(2,5-dioxo-2,5-dihydro-1H-pyrrol-1-yl)hexanoate with 2,5-dioxopyrrolidin-1-yl 2-(2,5-dioxo-2,5-dihydro-1H-pyrrol-1-yl)acetate. ¹H NMR (400 MHz, dimethyl sulfoxide-d₆) δ 10.02 (s, 1H), 8.38 (d, 1H), 8.14 (d, 1H), 8.03 (d, 1H), 7.82 (dd, 2H), 7.60 (t, 3H), 7.55-7.40 (m, 3H), 7.35 (td, 2H), 7.31-7.24 (m, 3H), 7.07 (s, 2H), 6.95 (d, 1H), 4.97 (d, 4H), 4.37 (ddd, 2H), 4.23-4.05 (m, 3H), 3.88 (t, 6H), 3.80 (d, 2H), 3.25 (d, 2H), 3.09-2.88 (m, 4H), 2.64 (s, 2H), 2.22 (dd, 2H), 2.09 (s, 3H), 2.02-1.49 (m, 5H), 1.47-0.89 (m, 12H), 0.83 (dd, 12H). MS (ESI) m/e 1523.5 (M−H)⁻.

### 2.117 Synthesis of 1-{[2-({3-[(4-{6-[8-(1,3-benzothiazol-2-ylcarbamoyl)-3,4-dihydroisoquinolin-2(1H)-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl]({[4-({N-[6-(2,5-dioxo-2,5-dihydro-1H-pyrrol-1-yl)hexanoyl]-D-valyl-N⁵-carbamoyl-D-ornithyl}amino)benzyl]oxy}carbonyl)amino}-1,2-dideoxy-D-arabino-hexitol

The title compound was prepared by substituting Example 1.77.2 for Example 1.2.9 in Example 2.1. ¹H NMR (400 MHz, dimethyl sulfoxide-d₆) δ ppm 12.85 (bs, 1H), 9.98 (s, 1H), 8.06 (d, 1H), 8.03 (d, 1H), 7.78 (t, 2H), 7.60 (m, 3H), 7.52-7.42 (m, 4H), 7.36 (q, 2H), 7.28 (s, 1H), 7.27 (d, 2H), 6.99 (s, 1H), 6.95 (d, 1H), 5.97 (bs, 1H), 5.00 (m, 2H), 4.95 (s, 2H), 4.39 (m, 1H), 4.19 (m, 2H), 3.88 (t, 2H), 3.79 (m, 4H), 3.58 (m, 4H), 3.46-3.33 (m, 10H), 3.26 (m, 4H), 3.01 (m, 2H), 2.94 (m, 1H), 2.14 (m, 2H), 2.09 (s, 3H), 1.96 (m, 1H), 1.69 (m, 2H), 1.59 (m, 1H), 1.47 (m, 4H), 1.35 (m, 4H), 1.28-1.03 (m, 10H), 0.95 (m, 2H), 0.82 (m, 12H). MS (ESI) m/e 1493 (M+H)⁺, 1491 (M−H)⁻.

### 2.118 Synthesis of N-[6-(2,5-dioxo-2,5-dihydro-1H-pyrrol-1-yl)hexanoyl]-L-valyl-N-{4-[({[2-({3-[(4-{6-[4-(1,3-benzothiazol-2-ylcarbamoyl)-2-oxidoisoquinolin-6-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](methyl)carbamoyl}oxy)methyl]phenyl}-N⁵-carbamoyl-L-ornithinamide

The title compound was prepared as described in Example 2.1, replacing Example 1.2.9 with Example 1.88.4. ¹H NMR (400 MHz, dimethyl sulfoxide-d₆) δ ppm 13.29 (s, 2H), 9.95 (s, 1H), 9.18 (s, 1H), 8.67 (s, 1H), 8.57-8.36 (m, 1H), 8.29-7.87 (m, 4H), 7.77 (dd, 2H), 7.56 (d, 2H), 7.53-7.41 (m, 2H), 7.24 (d, 2H), 6.95 (s, 2H), 5.95 (s, 1H), 4.94 (s, 2H), 4.35 (q, 1H), 4.15 (dd, 1H), 3.84 (s, 3H), 3.28 (dt, 4H), 3.06-2.77 (m, 3H), 2.19 (d, 3H), 2.17-1.80 (m, 3H), 1.74-0.88 (m, 22H), 0.79 (dd, 13H). MS (ESI) m/e 1368.4 (M−H)⁻.

### 2.119 Synthesis of N-({(3S,5S)-3-(2,5-dioxo-2,5-dihydro-1H-pyrrol-1-yl)-2-oxo-5-[(2-sulfoethoxy)methyl]pyrrolidin-1-yl}acetyl)-L-valyl-N-{4-[({[2-({3-[(4-{6-[8-(1,3-benzothiazol-2-ylcarbamoyl)naphthalen-2-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](2-sulfoethyl)carbamoyl}oxy)methyl]phenyl}-N⁵-carbamoyl-L-ornithinamide

### 2.119.1 (3R,7aS)-3-phenyltetrahydropyrrolo[1,2-c]oxazol-5(3H)-one

A mixture of (S)-5-(hydroxymethyl) pyrrolidin-2-one (25 g), benzaldehyde (25.5 g) and para-toluenesulfonic acid monohydrate (0.50 g) in toluene (300 mL) was heated to reflux using a Dean-Stark trap under a drying tube for 16 hours. The reaction was cooled to room temperature, and the solvent was decanted from the insoluble materials. The organic layer was washed with saturated aqueous sodium bicarbonate mixture (2×) and brine (1×). The organic layer was dried over sodium sulfate, filtered and concentrated under reduced pressure. The residue was purified by flash chromatography on silica gel, eluting with 35/65 heptane/ethyl acetate, to give the title compound. MS (DCI) m/e 204.0 (M+H)+.

### 2.119.2 (3R,6R,7aS)-6-bromo-3-phenyltetrahydropyrrolo[1,2-c]oxazol-5(3H)-one

To a cold (−77° C.) mixture of Example 2.119.1 (44.6 g) in tetrahydrofuran (670 mL) was added lithium bis(trimethylsilyl)amide (1.0M in hexanes, 250 mL) dropwise over 40 minutes, keeping Tᵣₓₙ<−73° C. The reaction was stirred at −77° C. for 2 hours, and bromine (12.5 mL) was added dropwise over 20 minutes, keeping Tᵣₓₙ<−64° C. The reaction was stirred at −77° C. for 75 minutes and was quenched by the addition of 150 mL cold 10% aqueous sodium thiosulfate mixture to the −77° C. reaction. The reaction was warmed to room temperature and partitioned between half-saturated aqueous ammonium chloride mixture and ethyl acetate. The layers were separated, and the organic layer was washed with water and brine, dried over sodium sulfate, filtered and concentrated under reduced pressure. The residue was purified by silica gel chromatography, eluting with a gradient of 80/20, 75/25, and 70/30 heptane/ethyl acetate to give the title compound. MS (DCI) m/e 299.0 and 301.0 (M+NH₃+H)⁺.

### 2.119.3 (3R,6S,7aS)-6-bromo-3-phenyltetrahydropyrrolo[1,2-c]oxazol-5(3H)-one

The title compound was isolated as a by-product from Example 2.119.2. MS (DCI) m/e 299.0 and 301.0 (M+NH₃+H)⁺.

### 2.119.4 (3R,6S,7aS)-6-azido-3-phenyltetrahydropyrrolo[1,2-c]oxazol-5(3H)-one

To a mixture of Example 2.119.2 (19.3 g) in N,N-dimethylformamide (100 mL) was added sodium azide (13.5 g). The reaction was heated to 60° C. for 2.5 hours. The reaction was cooled to room temperature and quenched by the addition of water (500 mL) and ethyl acetate (200 mL). The layers were separated, and the organic layer was washed brine. The combined aqueous layers were back-extracted with ethyl acetate (50 mL). The combined organic layers were dried with sodium sulfate, filtered and concentrated under reduced pressure. The residue was purified by silica gel chromatography, eluting with 78/22 heptane/ethyl acetate, to give the title compound. MS (DCI) m/e 262.0 (M+NH₃+H)⁺.

### 2.119.5 (3R,6S,7aS)-6-amino-3-phenyltetrahydropyrrolo[1,2-c]oxazol-5(3H)-one

To a mixture of Example 2.119.4 (13.5 g) in tetrahydrofuran (500 mL) and water (50 mL) was added polymer-supported triphenylphosphine (55 g). The reaction was mechanically stirred overnight at room temperature. The reaction was filtered through diatomaceous earth, eluting with ethyl acetate and toluene. The mixture was concentrated under reduced pressure, dissolved in dichloromethane (100 mL), dried with sodium sulfate, then filtered and concentrated to give the title compound, which was used in the subsequent step without further purification. MS (DCI) m/e 219.0 (M+H)⁺.

### 2.119.6 (3R,6S,7aS)-6-(dibenzylamino)-3-phenyltetrahydropyrrolo[1,2-c]oxazol-5(3H)-one

To a mixture of Example 2.119.5 (11.3 g) in N,N-dimethylformamide (100 mL) was added potassium carbonate (7.0 g), potassium iodide (4.2 g), and benzyl bromide (14.5 mL). The reaction was stirred at room temperature overnight and quenched by the addition of water and ethyl acetate. The layers were separated, and the organic layer was washed brine. The combined aqueous layers were back-extracted with ethyl acetate. The combined organic layers were dried with sodium sulfate, filtered and concentrated under reduced pressure. The residue was purified by silica gel chromatography, eluting with a gradient of 10 to 15% ethyl acetate in heptane to give a solid that was triturated with heptane to give the title compound. MS (DCI) m/e 399.1 (M+H)⁺.

### 2.119.7 (3S,5S)-3-(dibenzylamino)-5-(hydroxymethyl)pyrrolidin-2-one

To a mixture of Example 2.119.6 (13 g) in tetrahydrofuran (130 mL) was added para-toluene sulfonic acid monohydrate (12.4 g) and water (50 mL), and the reaction was heated to 65° C. for 6 days. The reaction was cooled to room temperature and quenched by the addition of saturated aqueous sodium bicarbonate and ethyl acetate. The layers were separated, and the organic layer was washed with brine. The combined aqueous layers were back-extracted with ethyl acetate. The combined organic layers were dried with sodium sulfate, filtered and concentrated under reduced pressure. The waxy solids were triturated with heptane (150 mL) to give the title compound. MS (DCI) m/e 311.1 (M+H)⁺.

### 2.119.8 (3S,5S)-5-(((tert-butyldimethylsilyl)oxy)methyl)-3-(dibenzylamino)pyrrolidin-2-one

To a mixture of Example 2.119.7 (9.3 g) and 1H-imidazole (2.2 g) in N,N-dimethylformamide was added tert-butylchlorodimethylsilane (11.2 mL, 50 weight % in toluene), and the reaction mixture was stirred overnight. The reaction mixture was quenched by the addition of water and ethyl ether. The layers were separated, and the organic layer was washed with brine. The combined aqueous layers were back-extracted with diethyl ether. The combined organic layers were dried with sodium sulfate, filtered and concentrated under reduced pressure. The residue was purified by silica gel chromatography, eluting with 35% ethyl acetate in heptane, to give the title compound. MS (DCI) m/e 425.1 (M+H)⁺.

### 2.119.9 tert-butyl 2-((3S,5S)-5-(((tert-butyldimethylsilyl)oxy)methyl)-3-(dibenzylamino)-2-oxopyrrolidin-1-yl)acetate

To a cold (0° C.) mixture of Example 2.119.8 (4.5 g) in tetrahydrofuran (45 mL) was added 95% sodium hydride (320 mg) in two portions. The cold mixture was stirred for 40 minutes, and tert-butyl 2-bromoacetate (3.2 mL) was added. The reaction was warmed to room temperature and stirred overnight. The reaction was quenched by the addition of water and ethyl acetate. The layers were separated, and the organic layer was washed with brine. The combined aqueous layers were back-extracted with ethyl acetate. The combined organic layers were dried with sodium sulfate, filtered and concentrated under reduced pressure. The residue was purified by silica gel chromatography, eluting with a gradient of 5-12% ethyl acetate in heptane, to give the title compound. MS (DCI) m/e 539.2 (M+H)⁺.

### 2.119.10 tert-butyl 2-((3S,5S)-3-(dibenzylamino)-5-(hydroxymethyl)-2-oxopyrrolidin-1-yl)acetate

To a mixture of Example 2.119.9 (5.3 g) in tetrahydrofuran (25 mL) was added tetrabutylammonium fluoride (11 mL, 1.0M in 95/5 tetrahydrofuran/water). The reaction was stirred at room temperature for one hour and then quenched by the addition of saturated aqueous ammonium chloride mixture, water and ethyl acetate. The layers were separated, and the organic layer was washed with brine. The combined aqueous layers were back-extracted with ethyl acetate. The combined organic layers were dried with sodium sulfate, filtered and concentrated under reduced pressure. The residue was purified by silica gel chromatography, eluting with 35% ethyl acetate in heptane, to give the title compound. MS (DCI) m/e 425.1 (M+H)⁺.

### 2.119.11 tert-butyl 2-((3S,5S)-5-((2-((4-((tert-butyldimethylsilyl)oxy)-2,2-dimethylbutoxy)sulfonyl)ethoxy)methyl)-3-(dibenzylamino)-2-oxopyrrolidin-1-yl)acetate

To a mixture of Example 2.119.10 (4.7 g) in dimethyl sulfoxide (14 mL) was added a mixture of 4-((tert-butyldiphenylsilyl)oxy)-2,2-dimethylbutyl ethenesulfonate (14.5 g) in dimethyl sulfoxide (14 mL). Potassium carbonate (2.6 g) and water (28 μL) were added, and the reaction was heated at 60° C. under nitrogen for one day. The reaction was cooled to room temperature, and then quenched by the addition of brine mixture, water and diethyl ether. The layers were separated, and the organic layer was washed with brine. The combined aqueous layers were back-extracted with diethyl ether. The combined organic layers were dried with sodium sulfate, filtered and concentrated under reduced pressure. The residue was purified by silica gel chromatography, eluting with a gradient of 15-25% ethyl acetate in heptane, to give the title compound. MS (ESI+) m/e 871.2 (M+H)⁺.

### 2.119.12 tert-butyl 2-((3S,5S)-3-amino-5-((2-((4-((tert-butyldimethylsilyl)oxy)-2,2-dimethylbutoxy)sulfonyl)ethoxy)methyl)-2-oxopyrrolidin-1-yl)acetate

Example 2.119.11 (873 mg) was dissolved in ethyl acetate (5 mL) and methanol (15 mL), and palladium hydroxide on carbon, 20% by wt (180 mg) was added. The reaction mixture was stirred under a hydrogen atmosphere (30 psi) at room temperature for 30 hours, then at 50° C. for one hour. The reaction was cooled to room temperature, filtered, and concentrated to give the desired product. MS (ESI+) m/e 691.0 (M+H)⁺.

### 2.119.13 4-(((3S,5S)-1-(2-(tert-butoxy)-2-oxoethyl)-5-((2-((4-((tert-butyldimethylsilyl)oxy)-2,2-dimethylbutoxy)sulfonyl)ethoxy)methyl)-2-oxopyrrolidin-3-yl)amino)-4-oxobut-2-enoic Acid

Maleic anhydride (100 mg) was dissolved in dichloromethane (0.90 mL), and a mixture of Example 2.119.12 (650 mg) in dichloromethane (0.90 mL) was added dropwise, then heated at 40° C. for 2 hours. The reaction mixture was directly purified by silica gel chromatography, eluting with a gradient of 1.0-2.5% methanol in dichloromethane containing 0.2% acetic acid. After concentrating the product-bearing fractions, toluene (10 mL) was added, and the mixture was concentrated again to give the title compound. MS (ESI−) m/e 787.3 (M−H)⁻.

### 2.119.14 tert-butyl 2-((3S,5S)-5-((2-((4-((tert-butyldimethylsilyl)oxy)-2,2-dimethylbutoxy)sulfonyl)ethoxy)methyl)-3-(2,5-dioxo-2,5-dihydro-1H-pyrrol-1-yl)-2-oxopyrrolidin-1-yl)acetate

Example 2.119.13 (560 mg) was slurried in toluene (7 mL), and triethylamine (220 μL) and sodium sulfate (525 mg) were added. The reaction was heated at reflux under a nitrogen atmosphere for 6 hours, and the reaction mixture was stirred at room temperature overnight. The reaction was filtered, and the solids were rinsed with ethyl acetate. The eluent was concentrated under reduced pressure, and the residue was purified by silica gel chromatography, eluting with 45/55 heptane/ethyl acetate to give the title compound.

### 2.119.15 2-((3S,5S)-3-(2,5-dioxo-2,5-dihydro-1H-pyrrol-1-yl)-2-oxo-5-((2-sulfoethoxy)methyl)pyrrolidin-1-yl)acetic Acid

Example 2.119.14 (1.2 g) was dissolved in trifluoroacetic acid (15 mL) and heated to 65-70° C. under nitrogen overnight. The trifluoroacetic acid was removed under reduced pressure. The residue was dissolved in acetonitrile (2.5 mL) and purified by preparative reverse-phase liquid chromatography on a Luna C18(2) AXIA column (250×50 mm, 10μ particle size) using a gradient of 5-75% acetonitrile containing 0.1% trifluoroacetic acid in water over 30 minutes, to give the title compound. MS (ESI−) m/e 375.2 (M−H)⁻.

### 2.119.16 3-(1-((3-(2-((((4-((S)-2-((S)-2-amino-3-methylbutanamido)-5-ureidopentanamido)benzyl)oxy)carbonyl)(2-sulfoethyl)amino)ethoxy)-5,7-dimethyladamantan-1-yl)methyl)-5-methyl-1H-pyrazol-4-yl)-6-(8-(benzo[d]thiazol-2-ylcarbamoyl)naphthalen-2-yl)picolinic Acid

The title compound was prepared by substituting Example 1.43.7 for Example 1.2.9 in Example 2.49.1. MS (ESI−) m/e 1252.4 (M−H)⁻.

### 2.119.17 N-({(3S,5S)-3-(2,5-dioxo-2,5-dihydro-1H-pyrrol-1-yl)-2-oxo-5-[(2-sulfoethoxy)methyl]pyrrolidin-1-yl}acetyl)-L-valyl-N-{4-[({[2-({3-[(4-{6-[8-(1,3-benzothiazol-2-ylcarbamoyl)naphthalen-2-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](2-sulfoethyl)carbamoyl}oxy)methyl]phenyl}-N⁵-carbamoyl-L-ornithinamide

Example 2.119.15 (7 mg) was dissolved in N,N-dimethylformamide (0.15 mL), and O-(7-azabenzotriazol-1-yl)-N,N,N′,N′-tetramethyluronium hexafluorophosphate (9 mg) and N,N-diisopropylethylamine (7 μL) were added. The mixture was stirred for 3 minutes at room temperature and added to a mixture of Example 2.119.16 (28 mg) and N,N-diisopropylethylamine (15 μL) in N,N-dimethylformamide (0.15 mL). After 1 hour, the reaction was diluted with N,N-dimethylformamide/water 1/1 (1.0 mL) and purified by reverse-phase chromatography (C18 column), eluting with 5-75% acetonitrile in 0.1% TFA water, to provide the title compound. ¹H NMR (500 MHz, dimethyl sulfoxide-d₆) δ ppm 9.95 (s, 1H), 9.02 (s, 1H), 8.37 (d, 1H), 8.22 (m, 2H), 8.18 (m, 2H), 8.08 (m, 2H), 8.03 (m, 1H), 7.96 (br d, 1H), 7.81 (d, 1H), 7.70 (t, 1H), 7.61 (br m, 3H), 7.48 (m, 2H), 7.37 (t, 1H), 7.27 (br m, 2H), 7.08 (s, 2H), 4.99 (br d, 3H), 4.68 (t, 1H), 4.39 (m, 1H), 4.20 (m, 2H), 4.04 (m, 1H), 3.87 (br d, 2H), 3.74 (br m, 1H) 3.65 (br t, 2H), 3.48 (br m, 4H), 3.43 (br m, 2H), 3.26 (br m, 2H), 3.00 (br m, 2H), 2.80 (m, 1H), 2.76 (m, 1H), 2.66 (br m, 2H), 2.36 (br m, 1H), 2.22 (s, 3H), 2.00 (m, 1H), 1.87 (m, 1H), 1.69 (br m, 1H), 1.62 (br m, 1H), 1.40 (br m, 4H), 1.31-1.02 (m, 10H), 0.96 (m, 2H), 0.85 (m, 12H). MS (ESI−) m/e 1610.3 (M−H)⁻.

### 2.120 Synthesis of N-{(2S)-2-(2,5-dioxo-2,5-dihydro-1H-pyrrol-1-yl)-3-[4-(2,5,8,11,14,17,20,23,26,29,32-undecaoxatetratriacontan-34-yloxy)phenyl]propanoyl}-L-valyl-N-{4-[({[2-({3-[(4-{6-[8-(1,3-benzothiazol-2-ylcarbamoyl)naphthalen-2-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](2-sulfoethyl)carbamoyl}oxy)methyl]phenyl}-N⁵-carbamoyl-L-ornithinamide

### 2.120.1 (S)-methyl 3-(4-(2,5,8,11,14,17,20,23,26,29,32-undecaoxatetratriacontan-34-yloxy)phenyl)-2-((tert-butoxycarbonyl)amino)propanoate

To a mixture of 2,5,8,11,14,17,20,23,26,29,32-undecaoxatetratriacontan-34-yl 4-methylbenzenesulfonate (82.48 g) and potassium carbonate (84.97 g) in acetonitrile (1.5 L) was added(S)-methyl 2-((tert-butoxycarbonyl)amino)-3-(4-hydroxyphenyl)propanoate (72.63 g), and the reaction mixture was stirred at 30° C. for 12 hours. After LC/MS indicated the starting material was consumed and the major product was the desired product, the reaction was filtered, and the filtrate was concentrated to afford the crude product which was purified by prep-HPLC to provide the title compound. MS (ESI): m/e 811 (M+H₂O)⁺.

### 2.120.2 3-(4-(2,5,8,11,14,17,20,23,26,29,32-undecaoxatetratriacontan-34-yloxy)phenyl)-2-((tert-butoxycarbonyl)amino)propanoic Acid

To a mixture of Example 2.120.1 (90.00 g) in tetrahydrofuran (1.5 L) and water (500 mL) was added lithium hydroxide monohydrate (14.27 g). The reaction mixture was stirred at 30° C. for 12 hours, and LC/MS indicated the starting material was consumed and the major product was the desired product. The reaction mixture was adjusted using aqueous HCl to pH=6, and the mixture was concentrated to provide the crude title compound. MS (ESI): m/e 778.3 (M−H)⁻.

### 2.120.3 3-(4-(2,5,8,11,14,17,20,23,26,29,32-undecaoxatetratriacontan-34-yloxy)phenyl)-2-aminopropanoic Acid

To a mixture of Example 2.120.2 (88.41 g) in dichloromethane (1.5 L) was added trifluoroacetic acid (100 mL) at 25° C. under N₂, and the reaction mixture was stirred at 40° C. for 12 hours. LC/MS indicated the starting material was consumed, and the major product was the desired product. The mixture was concentrated to afford the crude product which was purified by prep-HPLC provide the title compound as a trifluoroacetic acid salt. ¹H NMR (400 MHz, CDCl₃) δ ppm 7.20 (d, J=8.6 Hz, 2H), 6.93 (d, J=8.2 Hz, 2H), 4.22 (dd, J=5.5, 7.4 Hz, 1H), 4.14-4.06 (m, 2H), 3.84-3.79 (m, 2H), 3.68-3.50 (m, 40H), 3.33 (s, 3H), 3.21 (d, J=5.5 Hz, 1H), 3.12-3.05 (m, 1H). MS (ESI) m/e 680.1 (M+H)⁺.

### 2.120.4 4-((2-(4-(2,5,8,11,14,17,20,23,26,29,32-undecaoxatetratriacontan-34-yloxy)phenyl)-1-carboxyethyl)amino)-4-oxobut-2-enoic Acid

To a mixture of Example 2.120.3 (80.00 g) in dioxane (1 L) was added furan-2, 5-dione (35 g), and the reaction mixture was stirred at 120° C. for 4 hours. LC/MS indicated the starting material was consumed, and the major product was the desired product. The mixture was concentrated to afford crude title compound which was used without purification in the next step. MS (ESI) m/e 795.4 (M+H)⁺.

### 2.120.5 (S)-3-(4-(2,5,8,11,14,17,20,23,26,29,32-undecaoxatetratriacontan-34-yloxy)phenyl)-2-(2,5-dioxo-2,5-dihydro-1H-pyrrol-1-yl)propanoic Acid

To a mixture of Example 2.120.4 (96 g, crude) in toluene (1.5 L) and was added triethylamine (35.13 g), and the reaction mixture was stirred at 120° C. for 4 hours. LC/MS indicated the starting material was consumed, and the major product was the desired product. The reaction was filtered to isolate the organic phase, and the organics were concentrated to afford the crude product which was purified by prep-HPLC (Instrument: Shimadzu LC-20AP preparative HPLC, Column: Phenomenex Luna (2) C18 250*50 mm i.d. 10 u, Mobile phase: A for H₂O (0.09% trifluoroacetic acid) and B for CH₃CN, Gradient: B from 15% to 43% in 20 minutes, Flow rate: 80 ml/minute, Wavelength: 220 & 254 nm, Injection amount: 1 gram per injection), followed by SFC-HPLC to provide the title compound. ¹H NMR (400 MHz, CDCl₃) δ ppm 6.98 (d, 2H), 6.74 (d, 2H), 6.56 (s, 2H), 4.85 (dd, 1H), 4.03 (t, 2H), 3.84-3.76 (m, 2H), 3.71-3.66 (m, 2H), 3.65-3.58 (m, 39H), 3.55-3.50 (m, 2H), 3.41-3.30 (m, 4H). MS (ESI) m/e 760.3 (M+H)⁺.

### 2.120.6 N-{(2S)-2-(2,5-dioxo-2,5-dihydro-1H-pyrrol-1-yl)-3-[4-(2,5,8,11,14,17,20,23,26,29,32-undecaoxatetratriacontan-34-yloxy)phenyl]propanoyl}-L-valyl-N-{4-[({[2-({3-[(4-{6-[8-(1,3-benzothiazol-2-ylcarbamoyl)naphthalen-2-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](2-sulfoethyl)carbamoyl}oxy)methyl]phenyl}-N⁵-carbamoyl-L-ornithinamide

The title compound was prepared by substituting Example 2.120.5 for Example 2.119.15 in Example 2.119.17. ¹H NMR (400 MHz, dimethyl sulfoxide-d₆) δ ppm 10.03 (s, 1H), 9.02 (s, 1H), 8.37 (d, 1H), 8.22 (m, 3H), 8.16 (d, 1H), 8.12 (br m, 1H), 8.07 (d, 1H), 8.01 (d, 1H), 7.96 (br d, 1H), 7.81 (d, 1H), 7.70 (t, 1H), 7.59 (br m, 2H), 7.48 (m, 2H), 7.37 (t, 1H), 7.28 (d, 2H), 7.02 (d, 2H), 6.89 (s, 2H), 6.77 (d, 2H), 4.98 (br d, 2H), 4.79 (dd, 1H), 4.39 (br m, 1H), 4.23 (br m, 2H), 3.99 (br m, 2H), 3.88 (br m, 2H), 3.69 (br m, 4H), 3.55 (m, 4H), 3.50 (s, 32H), 3.42 (m, 4H), 3.27 (m, 4H), 3.23 (s, 3H), 3.20 (m, 1H), 3.03 (br m, 1H), 2.98 (m, 1H), 2.65 (br t, 2H), 2.22 (s, 3H), 1.97 (br m, 1H), 1.69 (br m, 1H), 1.61 (br m, 1H), 1.39 (m, 4H), 1.31-0.91 (m, 12H), 0.85 (m, 9H), 0.77 (d, 3H). MS (ESI) m/e 1993.7 (M−H)⁻.

### 2.121 Synthesis of N-({(3S,5S)-3-(2,5-dioxo-2,5-dihydro-1H-pyrrol-1-yl)-2-oxo-5-[(2-sulfoethoxy)methyl]pyrrolidin-1-yl}acetyl)-L-valyl-N-{4-[({[2-({3-[(4-{6-[8-(1,3-benzothiazol-2-ylcarbamoyl)-3,4-dihydroisoquinolin-2(1H)-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](2-sulfoethyl)carbamoyl}oxy)methyl]phenyl}-N⁵-carbamoyl-L-ornithinamide

The title compound was prepared by substituting Example 2.49.1 for Example 2.119.16 in Example 2.119.17. ¹H NMR (400 MHz, dimethyl sulfoxide-d₆) δ ppm 9.96 (s, 1H), 8.17 (br d, 1H), 8.03 (d, 2H), 7.79 (d, 1H), 7.61 (m, 3H), 7.55 (d, 1H), 7.45 (m, 2H), 7.37 (m, 3H), 7.27 (d, 2H), 7.08 (s, 2H), 6.98 (d, 1H), 4.97 (m, 4H), 4.68 (t, 1H), 4.37 (br m, 1H), 4.22 (br s, 1H), 4.17 (d, 1H), 4.03 (d, 1H), 3.89 (br t, 2H), 3.83 (br d, 2H), 3.74 (br m, 1H), 3.65 (t, 2H), 3.49 (m, 3H), 3.40 (br m, 4H), 3.25 (br m, 2H), 3.02 (br m, 4H), 2.80 (m, 2H), 2.67 (br m, 2H), 2.37 (br m, 1H), 2.10 (s, 3H), 1.99 (m, 1H), 1.86 (m, 1H), 1.69 (br m, 1H), 1.61 (br m, 1H), 1.52-0.91 (m, 16H), 0.85 (m, 12H). MS (ESI) m/e 1615.4 (M−H)⁻.

### 2.122 Synthesis of N-{(2S)-2-(2,5-dioxo-2,5-dihydro-1H-pyrrol-1-yl)-3-[4-(2,5,8,11,14,17,20,23,26,29,32-undecaoxatetratriacontan-34-yloxy)phenyl]propanoyl}-L-valyl-N-{4-[({[2-({3-[(4-{6-[8-(1,3-benzothiazol-2-ylcarbamoyl)-3,4-dihydroisoquinolin-2(1H)-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](2-sulfoethyl)carbamoyl}oxy)methyl]phenyl}-N⁵-carbamoyl-L-ornithinamide

To a mixture of Example 2.120.5 (19.61 mg), and O-(7-azabenzotriazol-1-yl)-N,N,N′,N′-tetramethyluronium hexafluorophosphate (9.81 mg) in N,N-dimethylformamide (0.8 mL) was added N,N-diisopropylethylamine (27.7 μL). The mixture was stirred for 5 minutes and added to a cold mixture of Example 2.112.2 in N,N-dimethylformamide (0.5 mL) at 0° C. The reaction mixture was stirred at 0° C. for 40 minutes, and purified by reverse-phase HPLC on a Gilson system (C18 column), eluting with 20-80% acetonitrile in water containing 0.1% trifluoroacetic acid, to give the title compound. ¹H NMR (400 MHz, dimethyl sulfoxide-d₆) δ 9.99 (s, 1H), 8.19 (d, 1H), 8.14-8.04 (m, 1H), 8.00 (dd, 1H), 7.75 (d, 1H), 7.62-7.52 (m, 3H), 7.49 (d, 1H), 7.46-7.37 (m, 2H), 7.36-7.29 (m, 2H), 7.28-7.21 (m, 3H), 6.99 (d, 2H), 6.92 (d, 1H), 6.85 (s, 2H), 6.79-6.71 (m, 2H), 4.94 (d, 3H), 4.76 (dd, 1H), 4.35 (d, 1H), 4.20 (t, 1H), 3.96 (dd, 2H), 3.85 (t, 2H), 3.77 (d, 2H), 3.66 (dd, 2H), 3.52 (dd, 2H), 3.50-3.47 (m, 2H), 3.39 (dd, 2H), 3.20 (s, 4H), 2.97 (t, 3H), 2.60 (t, 2H), 2.13-2.01 (m, 3H), 1.93 (s, 1H), 1.61 (d, 2H), 1.49-0.88 (m, 10H), 0.87-0.59 (m, 12H). MS (ESI) m/e 1998.7 (M−H)⁻.

### 2.123 Synthesis of (6S)-2,6-anhydro-6-(2-{2-[({[2-({3-[(4-{6-[8-(1,3-benzothiazol-2-ylcarbamoyl)-3,4-dihydroisoquinolin-2(1H)-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](2-sulfoethyl)carbamoyl}oxy)methyl]-5-({N-[(2,5-dioxo-2,5-dihydro-1H-pyrrol-1-yl)acetyl]-L-valyl-L-alanyl}amino)phenyl}ethyl)-L-gluconic Acid

### 2.123.1 (3R,4S,5R,6R)-3,4,5-tris(benzyloxy)-6-(benzyloxymethyl)-tetrahydropyran-2-one

To a mixture of (3R,4S,5R,6R)-3,4,5-tris(benzyloxy)-6-((benzyloxy)methyl)tetrahydro-2H-pyran-2-ol (75 g) in dimethyl sulfoxide (400 mL) at 0° C. was added acetic anhydride (225 mL). The mixture was stirred for 16 hours at room temperature before it was cooled to 0° C. A large volume of water was added, and stirring was stopped so that the reaction mixture was allowed to settle for 3 hours (the crude lactone migrated to the bottom of the flask). The supernatant was removed, and the crude mixture was diluted with ethyl acetate and was washed 3 times with water, neutralized with saturated aqueous mixture of NaHCO₃, and washed again twice with water. The organic layer was then dried over magnesium sulfate, filtered and concentrated to give the title compound. MS (ESI) m/e 561 (M+Na)⁺.

### 2.123.2 (3R,4S,5R,6R)-3,4,5-tris(benzyloxy)-6-(benzyloxymethyl)-2-ethynyl-tetrahydro-2H-pyran-2-ol

To a mixture of ethynyltrimethylsilane (18.23 g) in tetrahydrofuran (400 mL) under nitrogen and chilled in a dry ice/acetone bath (internal temp −65° C.) was added 2.5M BuLi in hexane (55.7 mL) dropwise, keeping the temperature below −60° C. The mixture was stirred in a cold bath for 40 minutes, followed by an ice-water bath (internal temp rose to 0.4° C.) for 40 minutes, and finally cooled to −75° C. again. A mixture of Example 2.123.1 (50 g) in tetrahydrofuran (50 mL) was added dropwise, keeping the internal temperature below −70° C. The mixture was stirred in a dry ice/acetone bath for additional 3 hours. The reaction was quenched with saturated aqueous NaHCO₃ mixture (250 mL). The mixture was allowed to warm to room temperature, extracted with ethyl acetate (3×300 mL), dried over MgSO₄, filtered, and concentrated in vacuo to give the title compound. MS (ESI) m/e 659 (M+Na)⁺.

### 2.123.3 trimethyl(((3S,4R,5R,6R)-3,4,5-tris(benzyloxy)-6-(benzyloxymethyl)-tetrahydro-2H-pyran-2-yl)ethynyl)silane

To a mixed mixture of Example 2.123.2 (60 g) in acetonitrile (450 mL) and dichloromethane (150 mL) at −15° C. in an ice-salt bath was added triethylsilane (81 mL) dropwise, followed by addition of boron trifluoride diethyl ether complex (40.6 mL) at such a rate that the internal temperature did not exceed −10° C. The mixture was then stirred at −15° C. to −10° C. for 2 hours. The reaction was quenched with saturated aqueous NaHCO₃ mixture (275 mL) and stirred for 1 hour at room temperature. The mixture was then extracted with ethyl acetate (3×550 mL). The extracts were dried over MgSO₄, filtered, and concentrated. The residue was purified by flash chromatography eluting with a gradient of 0% to 7% ethyl acetate/petroleum ether to give the title compound. MS (ESI) m/e 643 (M+Na)⁺.

### 2.123.4 (2R,3R,4R,5S)-3,4,5-tris(benzyloxy)-2-(benzyloxymethyl)-6-ethynyl-tetrahydro-2H-pyran

To a mixed mixture of Example 2.123.3 (80 g) in dichloromethane (200 mL) and methanol (1000 mL) was added 1N aqueous NaOH mixture (258 mL). The mixture was stirred at room temperature for 2 hours. The solvent was removed. The residue was then partitioned between water and dichloromethane. The extracts were washed with brine, dried over Na₂SO₄, filtered, and concentrated to give the title compound. MS (ESI) m/e 571 (M+Na)⁺.

### 2.123.5 (2R,3R,4R,5S)-2-(acetoxymethyl)-6-ethynyl-tetrahydro-2H-pyran-3,4,5-triyl Triacetate

To a mixture of Example 2.123.4 (66 g) in acetic anhydride (500 mL) cooled by an ice/water bath was added boron trifluoride diethyl ether complex (152 mL) dropwise. The mixture was stirred at room temperature for 16 hours, cooled with an ice/water bath and neutralized with saturated aqueous NaHCO₃ mixture. The mixture was extracted with ethyl acetate (3×500 mL), dried over Na₂SO₄, filtered, and concentrated in vacuo. The residue was purified by flash chromatography eluting with a gradient of 0% to 30% ethyl acetate/petroleum ether to give the title compound. MS (ESI) m/e 357 (M+H)⁺.

### 2.123.6 (3R,4R,5S,6R)-2-ethynyl-6-(hydroxymethyl)-tetrahydro-2H-pyran-3,4,5-triol

To a mixture of Example 2.123.5 (25 g) in methanol (440 mL) was added sodium methanolate (2.1 g). The mixture was stirred at room temperature for 2 hours, then neutralized with 4M HCl in dioxane. The solvent was removed, and the residue was adsorbed onto silica gel and loaded onto a silica gel column. The column was eluted with a gradient of 0 to 100% ethyl acetate/petroleum ether then 0% to 12% methanol/ethyl acetate to give the title compound. MS (ESI) m/e 211 (M+Na)⁺.

### 2.123.7 (2S,3S,4R,5R)-6-ethynyl-3,4,5-trihydroxy-tetrahydro-2H-pyran-2-carboxylic Acid

A three-necked round bottom flask was charged with Example 2.123.6 (6.00 g), KBr (0.30 g), tetrabutylammonium bromide (0.41 g) and 60 mL of saturated aqueous NaHCO₃ mixture. TEMPO ((2,2,6,6-tetramethylpiperidin-1-yl)oxyl, 0.15 g) in 60 mL dichloromethane was added. The mixture was stirred vigorously and cooled in an ice-salt bath to −2° C. internal temperature. A mixture of brine (12 mL), aqueous NaHCO₃ mixture (24 mL) and NaOCl (154 mL) was added dropwise such that the internal temperature was maintained below 2° C. The pH of the reaction mixture was maintained in the 8.2-8.4 range with the addition of solid Na₂CO₃. After a total of 6 hours, the reaction mixture was cooled to 3° C. internal temperature and ethanol (˜20 mL) was added dropwise. The mixture was stirred for ˜ 30 minutes. The mixture was transferred to a separatory funnel, and the dichloromethane layer was discarded. The pH of the aqueous layer was adjusted to 2-3 using 1 M aqueous HCl. The aqueous layer was then concentrated to dryness to afford a solid. Methanol (100 mL was) added to the dry solid, and the slurry was stirred for ˜30 minutes. The mixture was filtered over a pad of diatomaceous earth, and the residue in the funnel was washed with ˜100 mL of methanol. The filtrate was concentrated under reduced pressure to obtain the title compound.

### 2.123.8 (2S,3S,4R,5R)-methyl 6-ethynyl-3,4,5-trihydroxytetrahydro-2H-pyran-2-carboxylate

A 500 mL three-necked round bottom flask was charged with a suspension of Example 2.123.7 (6.45 g) in methanol (96 mL) and was cooled in an ice-salt-bath with internal temperature of −1° C. Neat thionyl chloride (2.79 mL) was carefully added. The internal temperature kept rising throughout the addition but did not exceed 10° C. The reaction was allowed to slowly warm up to 15-20° C. over 2.5 hours. After 2.5 hours, the reaction was concentrated to give the title compound.

### 2.123.9 (3S,4R,5S,6S)-2-ethynyl-6-(methoxycarbonyl)tetrahydro-2H-pyran-3,4,5-triyl Triacetate

To Example 2.123.8 (6.9 g) as a mixture in N,N-dimethylformamide (75 mL) was added 4-(dimethylamino)pyridine (0.17 g) and acetic anhydride (36.1 mL). The suspension was cooled in an ice-bath and pyridine (18.04 mL) was added via syringe over 15 minutes. The reaction was allowed to warm to room temperature overnight. Additional acetic anhydride (12 mL) and pyridine (6 mL) were added and stirring was continued for an additional 6 hours. The reaction was cooled in an ice-bath and 250 mL of saturated aqueous NaHCO₃ mixture was added and stirred for 1 hour. Water (100 mL) was added, and the mixture was extracted with ethyl acetate. The organic extract was washed twice with saturated CuSO₄ mixture, dried, filtered, and concentrated. The residue was purified by flash chromatography, eluting with 50% ethyl acetate/petroleum ether to give the title compound. ¹H NMR (500 MHz, methanol-d₄) δ ppm 5.29 (t, 1H), 5.08 (td, 2H), 4.48 (dd, 1H), 4.23 (d, 1H), 3.71 (s, 3H), 3.04 (d, 1H), 2.03 (s, 3H), 1.99 (s, 3H), 1.98 (s, 4H).

### 2.123.10 2-iodo-4-nitrobenzoic Acid

A 3 L fully jacketed flask equipped with a mechanical stirrer, temperature probe and an addition funnel under a nitrogen atmosphere, was charged with 2-amino-4-nitrobenzoic acid (69.1 g, Combi-Blocks) and sulfuric acid, 1.5 M aqueous (696 mL). The resulting suspension was cooled to 0° C. internal temperature, and a mixture of sodium nitrite (28.8 g) in water (250 mL) was added dropwise over 43 minutes with the temperature kept below 1° C. The reaction was stirred at ca. 0° C. for 1 hour. A mixture of potassium iodide (107 g) in water (250 mL) was added dropwise over 44 minutes with the internal temperature kept below 1° C. (Initially addition was exothermic and there was gas evolution). The reaction was stirred 1 hour at 0° C. The temperature was raised to 20° C. and then stirred at ambient temperature overnight. The reaction mixture became a suspension. The reaction mixture was filtered, and the collected solid was washed with water. The wet solid (˜ 108 g) was stirred in 10% sodium sulfite (350 ml, with ˜ 200 mL water used to wash in the solid) for 30 minutes. The suspension was acidified with concentrated hydrochloric acid (35 mL), and the solid was collected by filtration and washed with water. The solid was slurried in water (1 L) and re-filtered, and the solid was left to dry in the funnel overnight. The solid was then dried in a vacuum oven for 2 hours at 60° C. The resulting solid was triturated with dichloromethane (500 mL), and the suspension was filtered and washed with additional dichloromethane. The solid was air-dried to give the title compound

### 2.123.11 (2-iodo-4-nitrophenyl)methanol

A flame-dried 3 L 3-necked flask was charged with Example 2.123.10 (51.9 g) and tetrahydrofuran (700 mL). The mixture was cooled in an ice bath to 0.5° C., and borane-tetrahydrofuran complex (443 mL, 1M in THF) was added dropwise (gas evolution) over 50 minutes, reaching a final internal temperature of 1.3° C. The reaction mixture was stirred for 15 minutes, and the ice bath was removed. The reaction was left to come to ambient temperature over 30 minutes. A heating mantle was installed, and the reaction was heated to an internal temperature of 65.5° C. for 3 hours, and then allowed to cool to room temperature while stirring overnight. The reaction mixture was cooled in an ice bath to 0° C. and quenched by dropwise addition of methanol (400 mL). After a brief incubation period, the temperature rose quickly to 2.5° C. with gas evolution. After the first 100 mL are added over ˜ 30 minutes, the addition was no longer exothermic, and the gas evolution ceased. The ice bath was removed, and the mixture was stirred at ambient temperature under nitrogen overnight. The mixture was concentrated to a solid, dissolved in dichloromethane/methanol and adsorbed on to silica gel (˜ 150 g). The residue was loaded on a plug of silica gel (3000 mL) and eluted with dichloromethane to give the title compound.

### 2.123.12 (4-amino-2-iodophenyl)methanol

A 5 L flask equipped with a mechanical stirrer, heating mantle controlled by a JKEM temperature probe and a condenser was charged with Example 2.123.11 (98.83 g) and ethanol (2 L). The reaction was stirred rapidly, and iron (99 g) was added, followed by a mixture of ammonium chloride (20.84 g) in water (500 mL). The reaction was heated over the course of 20 minutes to an internal temperature of 80.3° C., where it began to reflux vigorously. The mantle was dropped until the reflux calmed. Thereafter, the mixture was heated to 80° C. for 1.5 hour. The reaction was filtered hot through a membrane filter, and the iron residue was washed with hot 50% ethyl acetate/methanol (800 mL). The eluent was passed through a diatomaceous earth pad, and the filtrate was concentrated. The residue was partitioned between 50% brine (1500 mL) and ethyl acetate (1500 mL). The layers were separated, and the aqueous layer was extracted with ethyl acetate (400 mL×3). The combined organic layers were dried over sodium sulfate, filtered and concentrated to give the title compound, which was used without further purification.

### 2.123.13 4-(((tert-butyldimethylsilyl)oxy)methyl)-3-iodoaniline

A 5 L flask with a mechanical stirrer was charged with Example 2.123.12 (88 g) and dichloromethane (2 L). The suspension was cooled in an ice bath to an internal temperature of 2.5° C., and tert-butylchlorodimethylsilane (53.3 g) was added portion-wise over 8 minutes. After 10 minutes, 1H-imidazole (33.7 g) was added portionwise to the cold reaction. The reaction was stirred 90 minutes while the internal temperature rose to 15° C. The reaction mixture was diluted with water (3 L) and dichloromethane (1 L). The layers were separated, and the organic layer was dried over sodium sulfate, filtered, and concentrated to an oil. The residue was purified by silica gel chromatography (1600 g silica gel), eluting a gradient of 0-25% ethyl acetate in heptane, to give the title compound as an oil.

### 2.123.14 (S)-2-((S)-2-((((9H-fluoren-9-yl)methoxy)carbonyl)amino)-3-methylbutanamido)propanoic Acid

To a mixture of (S)-2-((((9H-fluoren-9-yl)methoxy)carbonyl)amino)-3-methylbutanoic acid (6.5 g) in dimethoxyethane (40 mL) was added(S)-2-aminopropanoic acid (1.393 g) and sodium bicarbonate (1.314 g) in water (40 mL). Tetrahydrofuran (20 mL) was added to aid solubility. The resulting mixture was stirred at room temperature for 16 hours. Aqueous citric acid (15%, 75 mL) was added, and the mixture was extracted with 10% 2-propanol in ethyl acetate (2×100 mL). A precipitate formed in the organic layer. The combined organic layers were washed with water (2×150 mL). The organic layer was concentrated under reduced pressure and then triturated with diethyl ether (80 mL). After brief sonication, the title compound was collected by filtration. MS (ESI) m/e 411 (M+H)⁺.

### 2.123.15 (9H-fluoren-9-yl)methyl ((S)-1-(((S)-1-((4-(((tert-butyldimethylsilyl)oxy)methyl)-3-iodophenyl)amino)-1-oxopropan-2-yl)amino)-3-methyl-1-oxobutan-2-yl)carbamate

A mixture of Example 2.123.13 (5.44 g) and Example 2.123.14 (6.15 g) in a mixture of dichloromethane (70 mL) and methanol (35.0 mL) was added ethyl 2-ethoxyquinoline-1 (2H)-carboxylate (4.08 g), and the reaction was stirred overnight. The reaction mixture was concentrated and loaded onto silica gel, eluting with a gradient of 10% to 95% heptane in ethyl acetate followed by 5% methanol in dichloromethane. The product-containing fractions were concentrated, dissolved in 0.2% methanol in dichloromethane (50 mL), loaded onto silica gel and eluted with a gradient of 0.2% to 2% methanol in dichloromethane. The product containing fractions were collected to give the title compound. MS (ESI) m/e 756.0 (M+H)⁺.

### 2.123.16 (2S,3S,4R,5S,6S)-2-((5-((S)-2-((S)-2-((((9H-fluoren-9-yl)methoxy)carbonyl)amino)-3-methylbutanamido)propanamido)-2-(((tert-butyldimethylsilyl)oxy)methyl)phenyl)ethynyl)-6-(methoxycarbonyl)tetrahydro-2H-pyran-3,4,5-triyl Triacetate

A mixture of Example 2.123.9 (4.500 g), Example 2.123.15 (6.62 g), copper(I) iodide (0.083 g) and bis(triphenylphosphine)palladium(II) dichloride (0.308 g) were combined in vial and degassed. N,N-dimethylformamide (45 mL) and N-ethyl-N-isopropylpropan-2-amine (4.55 mL) were added, and the reaction vessel was flushed with nitrogen and stirred at room temperature overnight. The reaction was partitioned between water (100 mL) and ethyl acetate (250 mL). The layers were separated, and the organic layer was dried over magnesium sulfate, filtered, and concentrated. The residue was purified by silica gel chromatography, eluting with a gradient of 5% to 95% ethyl acetate in heptane. The product containing fractions were collected, concentrated and purified by silica gel chromatography, eluting with a gradient of 0.25% to 2.5% methanol in dichloromethane to give the title compound. MS (ESI) m/e 970.4 (M+H)⁺.

### 2.123.17 (2S,3S,4R,5S,6S)-2-(5-((S)-2-((S)-2-((((9H-fluoren-9-yl)methoxy)carbonyl)amino)-3-methylbutanamido)propanamido)-2-(((tert-butyldimethylsilyl)oxy)methyl) phenethyl)-6-(methoxycarbonyl)tetrahydro-2H-pyran-3,4,5-triyl Triacetate

Example 2.123.16 (4.7 g) and tetrahydrofuran (95 mL) were added to 5% Pt/C (2.42 g, wet) in a 50 mL pressure bottle and shaken for 90 minutes at room temperature under 50 psi of hydrogen. The reaction was filtered and concentrated to give the title compound. MS (ESI) m/e 974.6 (M+H)⁺.

### 2.123.18 (2S,3S,4R,5S,6S)-2-(5-((S)-2-((S)-2-((((9H-fluoren-9-yl)methoxy)carbonyl)amino)-3-methylbutanamido)propanamido)-2-(hydroxymethyl) phenethyl)-6-(methoxycarbonyl)tetrahydro-2H-pyran-3,4,5-triyl Triacetate

A mixture of Example 2.123.17 (5.4 g) in tetrahydrofuran (7 mL), water (7 mL) and glacial acetic acid (21 mL) was stirred overnight at room temperature. The reaction was diluted with ethyl acetate (200 mL) and washed with water (100 mL), saturated aqueous NaHCO₃ mixture (100 mL), brine (100 mL), dried over magnesium sulfate, filtered, and concentrated. The residue was purified by silica gel chromatography, eluting with a gradient of 0.5% to 5% methanol in dichloromethane, to give the title compound. MS (ESI) m/e 860.4 (M+H)⁺.

### 2.123.19 (2S,3S,4R,5S,6S)-2-(5-((S)-2-((S)-2-((((9H-fluoren-9-yl)methoxy)carbonyl)amino)-3-methylbutanamido)propanamido)-2-((((4-nitrophenoxy)carbonyl)oxy)methyl) phenethyl)-6-(methoxycarbonyl)tetrahydro-2H-pyran-3,4,5-triyl triacetate

To a mixture of Example 2.123.18 (4.00 g) and bis(4-nitrophenyl) carbonate (2.83 g) in acetonitrile (80 mL) was added N-ethyl-N-isopropylpropan-2-amine (1.22 mL) at room temperature. After stirring overnight, the reaction was concentrated, dissolved in dichloromethane (250 mL) and washed with saturated aqueous NaHCO₃ mixture (4×150 mL). The organic layer was dried over magnesium sulfate, filtered, and concentrated. The resulting foam was purified by silica gel chromatography, eluting with a gradient of 5% to 75% ethyl acetate in hexanes to give the title compound. MS (ESI) m/e 1025.5 (M+H)⁺.

### 2.123.20 3-(1-((3-(2-((((4-((R)-2-((R)-2-amino-3-methylbutanamido)propanamido)-2-(2-((2S,3R,4R,5S,6S)-6-carboxy-3,4,5-trihydroxytetrahydro-2H-pyran-2-yl)ethyl)benzyl)oxy)carbonyl)(2-sulfoethyl)amino)ethoxy)-5,7-dimethyladamantan-1-yl)methyl)-5-methyl-1H-pyrazol-4-yl)-6-(8-(benzo[d]thiazol-2-ylcarbamoyl)-3,4-dihydroisoquinolin-2(1H)-yl)picolinic Acid

To a cold (0° C.) mixture of Example 2.123.19 (70 mg) and Example 1.2.9 (58.1 mg) in N,N-dimethylformamide (4 mL) was added N-ethyl-N-isopropylpropan-2-amine (0.026 mL). The reaction was slowly warmed to room temperature and stirred overnight. To the reaction mixture was added water (1 mL) and LiOH H₂O (20 mg). The mixture was stirred at room temperature for 3 hours. The mixture was acidified with trifluoroacetic acid, filtered and purified by reverse-phase HPLC on a Gilson system (C18 column), eluting with 20-80% acetonitrile in water containing 0.1% trifluoroacetic acid, to give the title compound. MS (ESI) m/e 1564.4 (M−H)⁻.

### 2.123.21 (6S)-2,6-anhydro-6-(2-{2-[({[2-({3-[(4-{6-[8-(1,3-benzothiazol-2-ylcarbamoyl)-3,4-dihydroisoquinolin-2(1H)-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](2-sulfoethyl)carbamoyl}oxy)methyl]-5-({N-[(2,5-dioxo-2,5-dihydro-1H-pyrrol-1-yl)acetyl]-L-valyl-L-alanyl}amino)phenyl}ethyl)-L-gluconic Acid

The title compound was prepared as described in Example 2.54, replacing Example 2.49.1 with Example 2.123.20. ¹H NMR (500 MHz, dimethyl sulfoxide-d₆) δ ppm 12.86 (s, 1H), 9.92 (d, 1H), 8.35-8.19 (m, 2H), 8.04 (d, 1H), 7.80 (d, 1H), 7.61 (d, 1H), 7.57-7.32 (m, 8H), 7.28 (s, 1H), 7.22 (d, 1H), 7.08 (s, 2H), 6.95 (d, 1H), 5.12-4.91 (m, 5H), 4.39 (t, 1H), 4.32-4.19 (m, 1H), 4.12 (s, 2H), 3.89 (t, 2H), 3.80 (d, 2H), 3.14 (t, 1H), 3.06-2.87 (m, 4H), 2.69-2.58 (m, 4H), 2.37 (p, 1H), 2.09 (d, 4H), 2.04-1.91 (m, 4H), 1.54 (d, 1H), 1.40-0.99 (m, 20H), 0.99-0.74 (m, 16H). MS (ESI) m/e 1513.5 (M−H)⁻.

### 2.124 Synthesis of 3-{[2-({3-[(4-{6-[8-(1,3-benzothiazol-2-ylcarbamoyl)naphthalen-2-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl]({[4-(4-{[6-(2,5-dioxo-2,5-dihydro-1H-pyrrol-1-yl)hexanoyl]amino}butyl)-2-(beta-D-glucopyranuronosyloxy)benzyl]oxy}carbonyl)amino}propyl Beta-D-Glucopyranosiduronic Acid

### 2.124.1A (9H-fluoren-9-yl)methyl but-3-yn-1-ylcarbamate

A mixture of but-3-yn-1-amine hydrochloride (9 g) and N,N-diisopropylethylamine (44.7 mL) was stirred in dichloromethane (70 mL) and cooled to 0° C. A mixture of (9H-fluoren-9-yl)methyl carbonochloridate (22.06 g) in dichloromethane (35 mL) was added, and the reaction stirred for 2 hours. The reaction was concentrated, and the residue purified by silica gel chromatography, eluting with petroleum ether in ethyl acetate (10%-25%) to give the title compound. MS (ESI) m/e 314 (M+Na)⁺.

### 2.124.1B (3R,4S,5S,6S)-2-(2-formyl-5-iodophenoxy)-6-(methoxycarbonyl)tetrahydro-2H-pyran-3,4,5-triyl Triacetate

To a stirred solution of 2-hydroxy-4-iodobenzaldehyde (0.95 g) in acetonitrile (10 ml) was added (3R,4S,5S,6S)-2-bromo-6-(methoxycarbonyl)tetrahydro-2H-pyran-3,4,5-triyl triacetate (2.5 g) and silver oxide (2 g). The mixture was covered with aluminum foil and was stirred at room temperature overnight. After filtration through diatomaceous earth, the filtrate was washed with ethyl acetate, the solution was concentrated. The reaction mixture was purified by flash chromatography using an ISCO CombiFlash system, SF40-80 g column, eluted with 15-30% ethyl acetate/heptane (flow rate: 60 ml/min), to provide the title compound. MS (ESI) m/e 586.9 (M+Na)⁺.

### 2.124.2 (2S,3S,4S,5R,6S)-methyl 6-(5-(4-(((9H-fluoren-9-yl)methoxy)carbonylamino) but-1-ynyl)-2-formylphenoxy)-3,4,5-triacetoxy-tetrahydro-2H-pyran-2-carboxylate

Example 2.124.1B (2.7 g), Example 2.124.1A (2.091 g), bis(triphenylphosphine)palladium(II) chloride (0.336 g) and copper(I) iodide (0.091 g) were weighed into a vial and flushed with a stream of nitrogen. Triethylamine (2.001 mL) and tetrahydrofuran (45 mL) were added, and the reaction stirred at room temperature. After stirring for 16 hours, the reaction was diluted with ethyl acetate (200 mL) and washed with water (100 mL) and brine (100 mL). The organic layer was dried over magnesium sulfate, filtered, and concentrated. The residue was purified by silica gel chromatography, eluting with petroleum ether in ethyl acetate (10%-50%), to give the title compound. MS (ESI) m/e 750 (M+Na)⁺.

### 2.124.3 (2S,3S,4S,5R,6S)-methyl 6-(5-(4-(((9H-fluoren-9-yl)methoxy)carbonylamino)butyl)-2-formylphenoxy)-3,4,5-triacetoxy-tetrahydro-2H-pyran-2-carboxylate

Example 2.124.2 (1.5 g) and tetrahydrofuran (45 mL) were added to 10% Pd—C (0.483 g) in a 100 mL pressure bottle and stirred for 16 hours under 1 atm H₂ at room temperature. The reaction was filtered and concentrated to give the title compound. MS (ESI) m/e 754 (M+Na)⁺.

### 2.124.4 (2S,3S,4S,5R,6S)-methyl 6-(5-(4-(((9H-fluoren-9-yl)methoxy)carbonylamino)butyl)-2-(hydroxymethyl)phenoxy)-3,4,5-triacetoxy-tetrahydro-2H-pyran-2-carboxylate

A mixture of Example 2.124.3 (2.0 g) in tetrahydrofuran (7.00 mL) and methanol (7 mL) was cooled to 0° C. and NaBH₄ (0.052 g) was added in one portion. After 30 minutes, the reaction was diluted with ethyl acetate (150 mL) and water (100 mL). The organic layer was separated, washed with brine (100 mL), dried over magnesium sulfate, filtered, and concentrated. The residue was purified by silica gel chromatography, eluting with petroleum ether in ethyl acetate (10%-40%), to give the title compound. MS (ESI) m/e 756 (M+Na)⁺.

### 2.124.5 (2S,3S,4S,5R,6S)-methyl 6-(5-(4-(((9H-fluoren-9-yl)methoxy)carbonylamino)butyl)-2-(((4-nitrophenoxy)carbonyloxy)methyl)phenoxy)-3,4,5-triacetoxy-tetrahydro-2H-pyran-2-carboxylate

To a mixture of Example 2.124.4 (3.0 g) and bis(4-nitrophenyl) carbonate (2.488 g) in dry acetonitrile (70 mL) at 0° C. was added N,N-diisopropylethylamine (1.07 mL). After stirring at room temperature for 16 hours, the reaction was concentrated to give the residue, which was purified by silica gel chromatography, eluting with petroleum ether in ethyl acetate (10%-50%), to give the title compound. MS (ESI) m/e 921 (M+Na)⁺.

### 2.124.6 3-(1-((3-(2-((((4-(4-aminobutyl)-2-(((2R,3S,4R,5R,6R)-6-carboxy-3,4,5-trihydroxytetrahydro-2H-pyran-2-yl)oxy)benzyl)oxy)carbonyl)(3-(((2S,3S,4R,5R,6R)-6-carboxy-3,4,5-trihydroxytetrahydro-2H-pyran-2-yl)oxy)propyl)amino)ethoxy)-5,7-dimethyladamantan-1-yl)methyl)-5-methyl-1H-pyrazol-4-yl)-6-(8-(benzo[d]thiazol-2-ylcarbamoyl)naphthalen-2-yl)picolinic Acid

To a cold (0° C.) mixture of Example 2.124.5 (44 mg) and Example 1.87.3 (47.4 mg) in N,N-dimethylformamide (4 mL) was added N-ethyl-N-isopropylpropan-2-amine (0.026 mL). The reaction was slowly warmed to room temperature and stirred overnight. To the reaction mixture was added water (1 mL) and LiOH H₂O (20 mg). The mixture was stirred at room temperature for 3 hours. The mixture was acidified with trifluoroacetic acid, filtered and purified by reverse-phase HPLC on a Gilson system (C18 column), eluting with 20-80% acetonitrile in water containing 0.1% trifluoroacetic acid, to give the title compound. MS (ESI) m/e 1564.4 (M−H)⁻.

### 2.124.7 3-{[2-({3-[(4-{6-[8-(1,3-benzothiazol-2-ylcarbamoyl)naphthalen-2-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl]({[4-(4-{[6-(2,5-dioxo-2,5-dihydro-1H-pyrrol-1-yl)hexanoyl]amino}butyl)-2-(beta-D-glucopyranuronosyloxy)benzyl]oxy}carbonyl)amino}propyl beta-D-glucopyranosiduronic Acid

The title compound was prepared as described in Example 2.5.4, replacing Example 2.5.3 with Example 2.124.6. ¹H NMR (400 MHz, dimethyl sulfoxide-d₆) δ ppm 13.06 (s, 2H), 8.99 (s, 1H), 8.34 (dd, 1H), 8.25-8.09 (m, 3H), 8.08-8.02 (m, 1H), 7.98 (d, 1H), 7.89 (d, 1H), 7.78 (d, 1H), 7.66 (q, 2H), 7.50-7.41 (m, 2H), 7.37-7.31 (m, 1H), 7.14 (t, 1H), 6.94 (s, 2H), 6.90 (s, 1H), 6.82 (d, 1H), 5.14-5.02 (m, 2H), 4.97 (d, 1H), 4.19 (d, 1H), 3.85 (dd, 3H), 3.37-3.23 (m, 9H), 3.14 (t, 1H), 3.04-2.92 (m, 4H), 2.19 (s, 3H), 1.96 (t, 2H), 1.73 (s, 2H), 1.55-0.87 (m, 21H), 0.81 (d, 6H). MS (ESI) m/e 1564.4 (M−H)⁻.

### 2.125 Synthesis of N-{[(3S,5S)-3-(2,5-dioxo-2,5-dihydro-1H-pyrrol-1-yl)-5-(methoxymethyl)-2-oxopyrrolidin-1-yl]acetyl}-L-valyl-N-{4-[({[2-({3-[(4-{6-[8-(1,3-benzothiazol-2-ylcarbamoyl)-3,4-dihydroisoquinolin-2(1H)-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](2-sulfoethyl)carbamoyl}oxy)methyl]phenyl}-N⁵-carbamoyl-L-ornithinamide

### 2.125.1 tert-butyl 2-((3S,5S)-3-(dibenzylamino)-5-(methoxymethyl)-2-oxopyrrolidin-1-yl)acetate

To a mixture of Example 2.119.10 (1.4 g) in N,N-dimethylformamide (5 mL) was added iodomethane (0.8 mL). The reaction was cooled to 0° C., and 95% sodium hydride (80 mg) was added. After five minutes the cooling bath was removed, and the reaction stirred at room temperature for 2.5 hours. The reaction was quenched by the addition of water (20 mL) and ethyl acetate (40 mL). The layers were separated, and the organic layer was washed with brine. The combined aqueous layers were back-extracted with ethyl acetate (10 mL). The combined organic layers were dried with sodium sulfate, filtered and concentrated under reduced pressure. The residue was purified by silica gel chromatography, eluting with 80/20 heptane/ethyl acetate, to give the title compound. MS (DCI) m/e 439.2 (M+H)⁺.

### 2.125.2 tert-butyl 2-((3S,5S)-3-amino-5-(methoxymethyl)-2-oxopyrrolidin-1-yl)acetate

To a mixture of Example 2.125.1 (726 mg) in 2,2,2-trifluoroethanol (10 mL) was added palladium hydroxide on carbon (20% by wt, 150 mg). The reaction was stirred under a hydrogen atmosphere (50 psi) at room temperature for two hours. The reaction was filtered and concentrated to give the title compound. MS (DCI) m/e 259.0 (M+H)⁺.

### 2.125.3 4-(((3S,5S)-1-(2-(tert-butoxy)-2-oxoethyl)-5-(methoxymethyl)-2-oxopyrrolidin-3-yl)amino)-4-oxobut-2-enoic Acid

The title compound was prepared by substituting Example 2.125.2 for Example 2.119.12 in Example 2.119.13. MS (DCI) m/e 374.0 (M+NH₃+H)⁺.

### 2.125.4 tert-butyl 2-((3S,5S)-3-(2,5-dioxo-2,5-dihydro-1H-pyrrol-1-yl)-5-(methoxymethyl)-2-oxopyrrolidin-1-yl)acetate

The title compound was prepared by substituting Example 2.125.3 for Example 2.119.13 in Example 2.119.14. MS (DCI) m/e 356.0 (M+NH₃+H)⁺.

### 2.125.5 2-((3S,5S)-3-(2,5-dioxo-2,5-dihydro-1H-pyrrol-1-yl)-5-(methoxymethyl)-2-oxopyrrolidin-1-yl)acetic Acid

To a mixture of Example 2.125.4 (120 mg) in dichloromethane (8 mL) was added trifluoroacetic acid (4 mL). The reaction was stirred at room temperature for 90 minutes and then concentrated under reduced pressure. The residue was dissolved in acetonitrile (4 mL) and purified by preparative reverse-phase HPLC with a Luna C18(2) AXIA column, 250×50 mm, 10μ particle size, using a gradient of 5-75% acetonitrile in 0.1% trifluoroacetic acid in water over 30 minutes, to give the title compound. MS (DCI) m/e 300.0 (M+NH₃+H)⁺.

### 2.125.6 N-{[(3S,5S)-3-(2,5-dioxo-2,5-dihydro-1H-pyrrol-1-yl)-5-(methoxymethyl)-2-oxopyrrolidin-1-yl]acetyl}-L-valyl-N-{4-[({[2-({3-[(4-{6-[8-(1,3-benzothiazol-2-ylcarbamoyl)-3,4-dihydroisoquinolin-2(1H)-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](2-sulfoethyl)carbamoyl}oxy)methyl]phenyl}-N⁵-carbamoyl-L-ornithinamide

The title compound was prepared by substituting Example 2.125.5 for Example 2.119.15 and Example 2.49.1 for Example 2.119.16 in Example 2.119.17. ¹H NMR (400 MHz, dimethyl sulfoxide-d₆) δ ppm 9.98 (s, 1H), 8.19 (br d, 1H), 8.03 (d, 1H), 7.96 (d, 1H), 7.79 (d, 1H), 7.61 (m, 3H), 7.55 (d, 1H), 7.45 (m, 2H), 7.37 (m, 2H), 7.32 (s, 1H), 7.27 (d, 2H), 7.08 (s, 2H), 6.96 (d, 1H), 5.00 (m, 2H), 4.96 (s, 2H), 4.69 (t, 1H), 4.39 (br m, 1H), 4.28 (m, 1H), 4.20 (d, 1H), 3.88 (t, 3H), 3.81 (br m, 3H), 3.46 (m, 3H), 3.40 (m, 2H), 3.26 (br m, 2H), 3.25 (s, 3H), 3.01 (m, 3H), 2.96 (m, 1H), 2.65 (t, 2H), 2.36 (br m, 1H), 2.10 (s, 3H), 2.00 (m, 1H), 1.94 (m, 1H), 1.69 (br m, 1H), 1.59 (br m, 1H), 1.49-0.92 (m, 16H), 0.88 (d, 3H), 0.83 (m, 9H). MS (ESI) m/e 1521.5 (M−H)⁻.

### 2.126 Synthesis of (6S)-2,6-anhydro-6-(2-{2-[({[2-({3-[(4-{6-[8-(1,3-benzothiazol-2-ylcarbamoyl)-3,4-dihydroisoquinolin-2(1H)-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](2-sulfoethyl)carbamoyl}oxy)methyl]-5-({N-[6-(2,5-dioxo-2,5-dihydro-1H-pyrrol-1-yl)hexanoyl]-L-valyl-L-alanyl}amino)phenyl}ethyl)-L-gluconic Acid

The title compound was prepared as described in Example 2.123.21, replacing 2,5-dioxopyrrolidin-1-yl 2-(2,5-dioxo-2,5-dihydro-1H-pyrrol-1-yl)acetate with 2,5-dioxopyrrolidin-1-yl 6-(2,5-dioxo-2,5-dihydro-1H-pyrrol-1-yl)hexanoate. ¹H NMR (501 MHz, dimethyl sulfoxide-d₆) δ ppm 12.83 (s, 1H), 9.87 (s, 1H), 8.09 (d, 1H), 8.05-7.95 (m, 1H), 7.77 (d, 2H), 7.59 (d, 1H), 7.55-7.31 (m, 7H), 7.28 (s, 1H), 7.20 (d, 1H), 6.97 (s, 2H), 6.94 (d, 1H), 5.08-4.84 (m, 5H), 4.36 (p, 1H), 3.78 (d, 2H), 3.54 (t, 1H), 3.48-3.28 (m, 9H), 3.21 (s, 2H), 3.12 (t, 2H), 3.02-2.84 (m, 4H), 2.81-2.54 (m, 6H), 2.19-1.84 (m, 9H), 1.63-1.39 (m, 6H), 1.35 (s, 1H), 1.29-0.86 (m, 18H), 0.80 (td, 15H). MS (ESI) m/e 1568.4 (M−H)⁻.

### 2.127 Synthesis of 2-[({[2-({3-[(4-{6-[8-(1,3-benzothiazol-2-ylcarbamoyl)-3,4-dihydroisoquinolin-2(1H)-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](2-sulfoethyl)carbamoyl}oxy)methyl]-5-(4-{[3-(2,5-dioxo-2,5-dihydro-1H-pyrrol-1-yl)propanoyl]amino}butyl)phenyl Beta-D-Glucopyranosiduronic Acid

### 2.127.1 3-(1-((3-(2-((((4-(4-aminobutyl)-2-(((2S,3R,4S,5S,6S)-6-carboxy-3,4,5-trihydroxytetrahydro-2H-pyran-2-yl)oxy)benzyl)oxy)carbonyl)(2-sulfoethyl)amino)ethoxy)-5,7-dimethyladamantan-1-yl)methyl)-5-methyl-1H-pyrazol-4-yl)-6-(8-(benzo[d]thiazol-2-ylcarbamoyl)-3,4-dihydroisoquinolin-2(1H)-yl)picolinic Acid

To a mixture of Example 1.2.9 (0.030 g), Example 2.124.5 (0.031 g) and 1H-benzo[d][1,2,3]triazol-1-ol hydrate (5 mg) in N,N-dimethylformamide (0.5 mL) was added N-ethyl-N-isopropylpropan-2-amine (0.017 mL), and the reaction mixture was stirred for 3 hours. The reaction mixture was concentrated, dissolved in tetrahydrofuran (0.4 mL) and methanol (0.4 mL) and treated with lithium hydroxide hydrate (0.020 g) as a mixture in water (0.5 mL). After 1 hour, the reaction was quenched with 2,2,2-trifluoroacetic acid (0.072 mL), diluted with N,N-dimethylformamide:water (1:1) (1 mL) and purified by preparatory reverse-phase HPLC using a Gilson PLC 2020 system, eluting with a gradient of 5% to 75% acetonitrile/water. Product-containing fractions were combined and lyophilized to give to title compound. MS (ESI) m/e 1251.7 (M+H)⁺.

### 2.127.2 2-[({[2-({3-[(4-{6-[8-(1,3-benzothiazol-2-ylcarbamoyl)-3,4-dihydroisoquinolin-2(1H)-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](2-sulfoethyl)carbamoyl}oxy)methyl]-5-(4-{[3-(2,5-dioxo-2,5-dihydro-1H-pyrrol-1-yl)propanoyl]amino}butyl)phenyl Beta-D-Glucopyranosiduronic Acid

To a mixture of Example 2.127.1 (0.027 g) and 2,5-dioxopyrrolidin-1-yl 3-(2,5-dioxo-2,5-dihydro-1H-pyrrol-1-yl)propanoate (6.32 mg) in N,N-dimethylformamide (0.4 mL) was added N-ethyl-N-isopropylpropan-2-amine (0.017 mL), and the reaction was stirred for 1 hour at room temperature. The reaction was quenched with a mixture of 2,2,2-trifluoroacetic acid (0.038 mL), water (1.5 mL) and N,N-dimethylformamide (0.5 mL) and purified by preparatory reverse-phase HPLC on a Gilson 2020 system, using a gradient of 5% to 75% acetonitrile/water. The product-containing fractions were lyophilized to give the title compound. ¹H NMR (501 MHz, dimethyl sulfoxide-d₆) δ 12.84 (s, 1H), 8.03 (dd, 1H), 7.91-7.85 (m, 1H), 7.78 (d, 1H), 7.61 (dd, 1H), 7.52 (dd, 1H), 7.50-7.40 (m, 2H), 7.39-7.31 (m, 2H), 7.31 (s, 1H), 7.17 (dd, 1H), 6.99-6.90 (m, 4H), 6.83 (d, 1H), 5.15-5.04 (m, 2H), 5.05-4.96 (m, 1H), 4.95 (s, 2H), 3.91-3.83 (m, 4H), 3.81 (d, 3H), 3.58 (t, 2H), 3.42 (td, 3H), 3.33-3.24 (m, 5H), 3.00 (q, 4H), 2.68 (dt, 2H), 2.29 (t, 2H), 2.09 (d, 3H), 1.49 (d, 3H), 1.34 (td, 5H), 1.21 (dd, 5H), 1.15-1.07 (m, 2H), 1.07 (s, 4H), 0.95 (q, 1H), 0.82 (d, 6H). MS (ESI) m/e 1402.1 (M+H)⁺.

### 2.128 Synthesis of 2-[({[2-({3-[(4-{6-[8-(1,3-benzothiazol-2-ylcarbamoyl)-3,4-dihydroisoquinolin-2(1H)-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](2-sulfoethyl)carbamoyl}oxy)methyl]-5-[4-({(2S)-2-(2,5-dioxo-2,5-dihydro-1H-pyrrol-1-yl)-3-[4-(2,5,8,11,14,17,20,23,26,29,32-undecaoxatetratriacontan-34-yloxy)phenyl]propanoyl}amino)butyl]phenyl Beta-D-Glucopyranosiduronic Acid

A mixture of Example 2.120.5 (0.035 g), O-(7-azabenzotriazol-1-yl)-N,N,N′,N′-tetramethyluronium hexafluorophosphate (0.015 g) and N-ethyl-N-isopropylpropan-2-amine (0.015 mL) was stirred in N,N-dimethylformamide (0.4 mL) for 5 minutes. The mixture was added to a mixture of Example 2.127.1 (0.030 g) and N-ethyl-N-isopropylpropan-2-amine (0.015 mL) in N,N-dimethylformamide (0.4 mL) and stirred at room temperature for 3 hours. The reaction was diluted with a mixture of water (1.5 mL), N,N-dimethylformamide (0.5 mL) and 2,2,2-trifluoroacetic acid (0.034 mL) and purified by preparatory reverse-phase HPLC on a Gilson 2020 system, using a gradient of 5% to 85% acetonitrile/water. The product-containing fractions were lyophilized to give the title compound. ¹H NMR (400 MHz, dimethyl sulfoxide-d₆) δ 12.83 (s, 1H), 8.04-7.93 (m, 2H), 7.76 (d, 1H), 7.58 (dd, 1H), 7.53-7.36 (m, 3H), 7.37-7.25 (m, 3H), 7.15 (d, 1H), 6.97-6.88 (m, 4H), 6.87 (d, 2H), 6.85-6.77 (m, 1H), 6.76-6.69 (m, 2H), 5.13-4.96 (m, 3H), 4.92 (s, 2H), 3.95 (dd, 2H), 3.84 (d, 2H), 3.78 (s, 8H), 3.69-3.60 (m, 2H), 3.47 (d, 38H), 3.48-3.35 (m, 6H), 3.20 (s, 8H), 3.10 (dd, 2H), 2.98 (t, 2H), 2.69-2.60 (m, 2H), 2.50 (d, 1H), 2.06 (s, 3H), 1.49 (t, 2H), 1.35 (s, 4H), 1.21 (d, 4H), 1.05 (s, 6H), 0.79 (d, 6H). MS (ESI) m/e 1991.6 (M−H)⁻.

### 2.129 Synthesis of (6S)-2,6-anhydro-6-(2-{2-[({[2-({3-[(4-{6-[8-(1,3-benzothiazol-2-ylcarbamoyl)-3,4-dihydroisoquinolin-2(1H)-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](2-sulfoethyl)carbamoyl}oxy)methyl]-5-[(N-{(2S)-2-(2,5-dioxo-2,5-dihydro-1H-pyrrol-1-yl)-3-[4-(2,5,8,11,14,17,20,23,26,29,32-undecaoxatetratriacontan-34-yloxy)phenyl]propanoyl}-L-valyl-L-alanyl)amino]phenyl}ethyl)-L-gluconic Acid

A mixture of Example 2.120.5 (0.033 g), O-(7-azabenzotriazol-1-yl)-N,N,N′,N′-tetramethyluronium hexafluorophosphate (0.014 g) and N-ethyl-N-isopropylpropan-2-amine (0.015 mL) was stirred in N,N-dimethylformamide (0.4 mL) for 5 minutes. This mixture was added to a mixture of Example 2.123.20 (0.032 g) and N-ethyl-N-isopropylpropan-2-amine (0.015 mL) in N,N-dimethylformamide (0.4 mL) and stirred at room temperature for 3 hours. The reaction was diluted with a mixture of water (1.5 mL), N,N-dimethylformamide (0.5 mL) and 2,2,2-trifluoroacetic acid (0.033 mL) and purified by preparatory reverse-phase HPLC on a Gilson 2020 system, using a gradient of 5% to 85% acetonitrile/water. The product-containing fractions were lyophilized to give the title compound. ¹H NMR (501 MHz, dimethyl sulfoxide-d₆) δ 9.90 (d, 1H), 8.25 (d, 1H), 8.12 (m, 1), 8.01 (m, 1H), 1.78 (m, 1H), 7.59 (d, 1H), 7.53-7.40 (m, 4H), 7.43-7.30 (m, 4H), 7.27 (s, 1H), 7.18 (d, 2H), 7.06 (s, 1H), 7.00 (d, 2H), 6.97-6.91 (m, 2H), 6.87 (s, 2H), 6.76 (d, 2H), 5.02-4.92 (m, 4H), 4.77 (dd, 1H), 4.20 (t, 1H), 3.98 (dd, 2H), 3.86 (t, 2H), 3.78 (d, 2H), 3.70-3.65 (m, 2H), 3.54 (s, 2H), 3.55-3.45 (m, 38H), 3.45-3.37 (m, 2H), 3.35-3.25 (m, 2H), 3.21 (s, 4H), 3.17-3.06 (m, 2H), 2.99 (t, 2H), 2.73 (s, 2H), 2.61 (s, 4H), 2.07 (d, 4H), 2.01 (s, 2H), 1.94 (s, 2H), 1.54 (s, 2H), 1.27 (d, 4H), 1.22 (s, 2H), 1.11 (s, 6H), 1.08-0.99 (m, 2H), 0.90-0.79 (m, 6H), 0.76 (d, 6H). MS (ESI) m/e 705.6 (M−3H)³⁻.

### 2.130 Synthesis of 6-(8-(benzo[d]thiazol-2-ylcarbamoyl)-3,4-dihydroisoquinolin-2(1H)-yl)-3-(1-((3-(2-((((2-(2-((2S,3R,4R,5S,6S)-6-carboxy-3,4,5-trihydroxytetrahydro-2H-pyran-2-yl)ethyl)-4-((S)-2-((S)-2-(2-((3S,5S)-3-(2,5-dioxo-2,5-dihydro-1H-pyrrol-1-yl)-2-oxo-5-((2-sulfoethoxy)methyl)pyrrolidin-1-yl)acetamido)-3-methylbutanamido)propanamido)benzyl)oxy)carbonyl)(2-sulfoethyl)amino)ethoxy)-5,7-dimethyladamantan-1-yl)methyl)-5-methyl-1H-pyrazol-4-yl)picolinic Acid

The title compound was prepared by substituting Example 2.123.20 for Example 2.119.16 in Example 2.119.17. ¹H NMR (400 MHz, dimethyl sulfoxide-d₆) δ ppm 9.85 (s, 1H), 8.17 (br d, 1H), 8.01 (d, 2H), 7.77 (d, 1H), 7.59 (d, 1H), 7.53 (d, 1H), 7.43 (m, 4H), 7.34 (m, 3H), 7.19 (d, 1H), 7.06 (s, 2H), 6.96 (d, 1H), 4.99 (m, 2H), 4.95 (s, 2H), 4.63 (t, 1H), 4.36 (t, 1H), 4.19 (br m, 1H), 4.16 (d, 1H), 3.98 (d, 1H), 3.87 (br t, 2H), 3.81 (br d, 2H), 3.73 (brm, 1H), 3.63 (t, 2H), 3.53 (m, 2H), 3.44 (m, 4H), 3.31 (t, 2H), 3.21 (br m, 2H), 3.17 (m, 2H), 3.00 (m, 2H), 2.92 (br m, 1H), 2.75 (m, 3H), 2.65 (br m, 3H), 2.35 (br m, 1H), 2.07 (s, 3H), 1.98 (br m, 2H), 1.85 (m, 1H), 1.55 (br m, 1H), 1.34 (br m, 1H), 1.26 (br m, 6H), 1.09 (br m, 7H), 0.93 (br m, 1H), 0.87, 0.83, 0.79 (all d, total 12H). MS (ESI) m/e 1733.4 (M−H)⁻.

### 2.131 Synthesis of 6-(8-(benzo[d]thiazol-2-ylcarbamoyl)-3,4-dihydroisoquinolin-2(1H)-yl)-3-(1-((3-(2-((((2-(((2S,3R,4S,5S,6S)-6-carboxy-3,4,5-trihydroxytetrahydro-2H-pyran-2-yl)oxy)-4-(4-(2-((3S,5S)-3-(2,5-dioxo-2,5-dihydro-1H-pyrrol-1-yl)-2-oxo-5-((2-sulfoethoxy)methyl)pyrrolidin-1-yl)acetamido)butyl)benzyl)oxy)carbonyl)(2-sulfoethyl)amino)ethoxy)-5,7-dimethyladamantan-1-yl)methyl)-5-methyl-1H-pyrazol-4-yl)picolinic Acid

The title compound was prepared by substituting Example 2.127.1 for Example 2.119.16 in Example 2.119.17. ¹H NMR (500 MHz, dimethyl sulfoxide-d₆) δ ppm 8.02 (d, 1H), 7.82 (br t, 1H), 7.77 (d, 1H), 7.60 (d, 1H), 7.53 (br d, 1H), 7.45 (ddd, 1H), 7.42 (d, 1H), 7.36 (d, 1H), 7.35 (s, 1H), 7.33 (m, 1H), 7.15 (d, 1H), 7.05 (s, 2H), 6.97 (d, 1H), 6.94 (s, 1H), 6.83 (d, 1H), 5.07 (br m, 2H), 5.00 (d, 1H), 4.95 (s, 2H), 4.69 (t, 1H), 4.04 (d, 2H), 3.87 (m, 3H), 3.82 (m, 3H), 3.73 (br m, 1H), 3.61 (m, 2H), 3.47 (br m, 3H), 3.40 (m, 4H), 3.29 (m, 4H), 3.06 (br m, 2H), 3.00 (t, 2H), 2.73 (br m, 2H) 2.69 (br m, 2H), 2.52 (br t, 2H), 2.35 (br m, 1H), 2.08 (s, 3H), 1.81 (m, 1H), 1.53 (br m, 2H), 1.40 (m, 2H), 1.35 (br m, 2H), 1.29-0.88 (br m, 10H), 0.82, 0.80 (both s, total 6H). MS (ESI−) m/e 1607.5 (M−H)⁻.

### 2.132 Synthesis of 2-[({[2-({3-[(4-{6-[8-(1,3-benzothiazol-2-ylcarbamoyl)-3,4-dihydroisoquinolin-2(1H)-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](2-sulfoethyl)carbamoyl}oxy)methyl]-5-(4-{[(2,5-dioxo-2,5-dihydro-1H-pyrrol-1-yl)acetyl]amino}butyl)phenyl Beta-D-Glucopyranosiduronic Acid

To a mixture of Example 2.127.1 (0.032 g) in N,N-dimethylformamide (0.4 mL) was added N-ethyl-N-isopropylpropan-2-amine (0.025 mL), and the mixture cooled to 0° C. 2,5-Dioxopyrrolidin-1-yl 2-(2,5-dioxo-2,5-dihydro-1H-pyrrol-1-yl)acetate (8.86 mg) was added in one portion and stirred at 0° C. for 45 minutes. The reaction was diluted with a mixture of water (1.5 mL), N,N-dimethylformamide (0.5 mL) and 2,2,2-trifluoroacetic acid (0.036 mL) and was purified by preparatory reverse-phase HPLC on a Gilson 2020 system, using a gradient of 5% to 75% acetonitrile/water. The product-containing fractions were lyophilized to give the title compound. ¹H NMR (501 MHz, dimethyl sulfoxide-d₆) δ 12.86 (s, 1H), 8.06 (s, 1H), 8.02 (dd, 1H), 7.77 (d, 1H), 7.60 (dd, 1H), 7.51 (dd, 1H), 7.49-7.39 (m, 2H), 7.38-7.28 (m, 3H), 7.17 (dd, 1H), 7.06 (d, 2H), 6.98-6.89 (m, 2H), 6.83 (d, 1H), 5.13-5.03 (m, 2H), 5.04-4.96 (m, 1H), 4.94 (s, 2H), 3.97 (s, 2H), 3.90-3.77 (m, 6H), 3.50 (s, 1H), 3.50-3.41 (m, 2H), 3.41 (dt, 3H), 3.28 (dt, 4H), 3.06-2.96 (m, 4H), 2.66 (dt, 2H), 2.51 (s, 2H), 2.08 (d, 3H), 1.52 (s, 2H), 1.42-1.32 (m, 4H), 1.23 (d, 4H), 1.11 (q, 2H), 1.06 (s, 4H), 0.81 (d, 6H). MS (ESI) m/e 1388.0 (M+H)⁺.

### 2.133 Synthesis of 2-[({[2-({3-[(4-{6-[8-(1,3-benzothiazol-2-ylcarbamoyl)naphthalen-2-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](2-sulfoethyl)carbamoyl}oxy)methyl]-5-(4-{[(2,5-dioxo-2,5-dihydro-1H-pyrrol-1-yl)acetyl]amino}butyl)phenyl Beta-D-Glucopyranosiduronic Acid

### 2.133.1 3-(1-((3-(2-((((4-(4-aminobutyl)-2-(((2S,3R,4S,5S,6S)-6-carboxy-3,4,5-trihydroxytetrahydro-2H-pyran-2-yl)oxy)benzyl)oxy)carbonyl)(2-sulfoethyl)amino)ethoxy)-5,7-dimethyladamantan-1-yl)methyl)-5-methyl-1H-pyrazol-4-yl)-6-(8-(benzo[d]thiazol-2-ylcarbamoyl)naphthalen-2-yl)picolinic Acid

To a mixture of Example 2.124.5 (0.060 g), Example 1.43.7 (0.056 g) and 1H-benzo[d][1,2,3]triazol-1-ol (8 mg) in dimethyl sulfoxide (0.5 mL) was added N-ethyl-N-isopropylpropan-2-amine (0.056 mL), and the reaction was stirred at room temperature for 3 hours. The reaction was treated with a mixture of lithium hydroxide hydrate (0.026 g) in water (1 mL) and stirred for 30 minutes. Methanol (0.5 mL) was added to the reaction and stirring was continued for 30 minutes. Diethylamine (0.033 mL) was added to the reaction and stirring was continued overnight. The reaction was quenched with 2,2,2-trifluoroacetic acid (0.120 mL) and purified by preparatory reverse-phase HPLC on a Gilson 2020 system, using a gradient of 5% to 75% acetonitrile/water. The product-containing fractions were lyophilized to give the title compound. MS (ESI) m/e 1247.7 (M+H)⁺.

### 2.133.2 2-[({[2-({3-[(4-{6-[8-(1,3-benzothiazol-2-ylcarbamoyl)naphthalen-2-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](2-sulfoethyl)carbamoyl}oxy)methyl]-5-(4-{[(2,5-dioxo-2,5-dihydro-1H-pyrrol-1-yl)acetyl]amino}butyl)phenyl Beta-D-Glucopyranosiduronic Acid

To a mixture of Example 2.133.1 (0.030 g) in N,N-dimethylformamide (0.400 mL) was added N-ethyl-N-isopropylpropan-2-amine (0.023 mL) and the mixture was cooled to 0° C. 2,5-Dioxopyrrolidin-1-yl 2-(2,5-dioxo-2,5-dihydro-1H-pyrrol-1-yl)acetate (8.34 mg) was added in one portion and the mixture was stirred at 0° C. for 30 minutes. The reaction was diluted with a mixture of water (1.5 mL), N,N-dimethylformamide (0.5 mL) and 2,2,2-trifluoroacetic acid (0.034 mL) and was purified by preparatory reverse-phase HPLC on a Gilson 2020 system, using a gradient of 5% to 75% acetonitrile/water. The product-containing fractions were lyophilized to give the title compound. ¹H NMR (400 MHz, dimethyl sulfoxide-d₆) δ 13.08 (s, 1H), 9.01 (s, 1H), 8.39-8.31 (m, 1H), 8.25-8.11 (m, 3H), 8.06 (d, 2H), 7.99 (d, 1H), 7.94 (d, 1H), 7.79 (d, 1H), 7.68 (t, 1H), 7.51-7.42 (m, 1H), 7.46 (s, 1H), 7.35 (t, 1H), 7.22-7.13 (m, 1H), 7.06 (d, 2H), 6.93 (d, 1H), 6.83 (d, 1H), 5.15-5.00 (m, 2H), 4.99 (d, 1H), 3.97 (s, 2H), 3.86 (d, 3H), 3.42 (d, 4H), 3.29 (d, 5H), 3.03 (p, 2H), 2.72-2.62 (m, 2H), 2.51 (d, 3H), 2.21 (s, 3H), 1.51 (q, 2H), 1.37 (q, 4H), 1.24 (d, 4H), 1.10 (s, 5H), 0.83 (d, 6H), 0.61 (s, 2H). MS (ESI) m/e 1383.0 (M+H)⁺.

### 2.134 Synthesis of 2-[({[2-({3-[(4-{6-[8-(1,3-benzothiazol-2-ylcarbamoyl)naphthalen-2-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](2-sulfoethyl)carbamoyl}oxy)methyl]-5-[4-({(2S)-2-(2,5-dioxo-2,5-dihydro-1H-pyrrol-1-yl)-3-[4-(2,5,8,11,14,17,20,23,26,29,32-undecaoxatetratriacontan-34-yloxy)phenyl]propanoyl}amino)butyl]phenyl Beta-D-Glucopyranosiduronic Acid

A mixture of Example 2.120.5 (0.028 g), O-(7-azabenzotriazol-1-yl)-N,N,N′,N′-tetramethyluronium hexafluorophosphate (0.013 g) and N-ethyl-N-isopropylpropan-2-amine (0.015 mL) were stirred in N,N-dimethylformamide (0.4 mL) for 5 minutes. The mixture was added to a mixture of Example 2.133.1 (0.030 g) and N-ethyl-N-isopropylpropan-2-amine (0.015 mL) in N,N-dimethylformamide (0.4 mL) and was stirred at room temperature for 1 hour. The reaction was diluted with a mixture of water (1.5 mL), N,N-dimethylformamide (0.5 mL) and 2,2,2-trifluoroacetic acid (0.042 mL) and was purified by preparatory reverse-phase HPLC on a Gilson 2020 system, using a gradient of 5% to 75% acetonitrile/water. The product-containing fractions were lyophilized to give the title compound. ¹H NMR (400 MHz, dimethyl sulfoxide-d₆) δ 9.01 (s, 1H), 8.35 (dd, 1H), 8.27-8.13 (m, 3H), 8.06 (d, 1H), 8.00 (d, 1H), 7.94 (d, 1H), 7.79 (d, 1H), 7.73-7.64 (m, 1H), 7.53-7.43 (m, 2H), 7.42-7.32 (m, 1H), 7.17 (d, 1H), 7.06 (s, 1H), 7.04-6.91 (m, 3H), 6.89 (d, 2H), 6.83 (d, 1H), 6.74 (d, 1H), 5.16-4.93 (m, 4H), 4.63 (dd, 2H), 3.96 (t, 2H), 3.86 (d, 4H), 3.66 (s, 4H), 3.55-3.46 (m, 36H), 3.45-3.35 (m, 8H), 3.35-3.24 (m, 6H), 3.21 (s, 2H), 3.11 (s, 2H), 2.99 (d, 2H), 2.83-2.59 (m, 3H), 2.52 (d, 2H), 2.21 (s, 3H), 1.57-0.86 (m, 14H), 0.83 (d, 4H). MS (ESI) m/e 1986.6 (M−H)⁻.

### 2.135 Synthesis of N-[(2,5-dioxo-2,5-dihydro-1H-pyrrol-1-yl)acetyl]-L-valyl-N-{4-[({[2-({3-[(4-{6-[8-(1,3-benzothiazol-2-ylcarbamoyl)-3,4-dihydroisoquinolin-2(1H)-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](2-sulfoethyl)carbamoyl}oxy)methyl]-3-(4-carboxybutyl)phenyl}-L-alaninamide 2.135.1 methyl 4-((tert-butoxycarbonyl)amino)-2-iodobenzoate

3-Iodo-4-(methoxycarbonyl)benzoic acid (9 g) was dissolved in tert-butanol (100 mL), and diphenyl phosphorazidate (7.6 mL) and triethylamine (4.9 mL) were added. The mixture was heated to 83° C. (internal temperature) overnight. The mixture was concentrated to dryness and purified by flash chromatography, eluting with a gradient of 0% to 20% ethyl acetate in heptane to give the title compound. MS (ESI) m/e 377.9 (M+H)⁺.

### 2.135.2 methyl 4-amino-2-iodobenzoate

Example 2.135.1 (3 g) was stirred in dichloromethane (30 mL) and trifluoroacetic acid (10 mL) at room temperature for 1.5 hours. The reaction was concentrated to dryness and partitioned between water (adjusted to pH 1 with hydrochloric acid) and diethyl ether. The layers were separated, and the aqueous layer was washed with aqueous sodium bicarbonate mixture, dried over sodium sulfate, filtered and concentrated to dryness. The resulting solid was triturated with toluene to give the title compound. MS (ESI) m/e 278.0 (M+H)⁺.

### 2.135.3 methyl 4-((S)-2-((S)-2-((((9H-fluoren-9-yl)methoxy)carbonyl)amino)-3-methylbutanamido)propanamido)-2-iodobenzoate

A flask was charged with Example 2.135.2 (337 mg) and Example 2.123.14 (500 mg). Ethyl acetate (18 mL) was added followed by pyridine (0.296 mL). The resulting suspension was chilled in an ice bath, and 2,4,6-tripropyl-1,3,5,2,4,6-trioxatriphosphinane 2,4,6-trioxide (50% mixture in ethyl acetate, 1.4 mL) was added dropwise. Stirring was continued at 0° C. for 45 minutes, and the reaction was placed in a −20° C. freezer overnight. The reaction was allowed to warm to room temperature and was quenched with water. The layers were separated, and the aqueous layer was extracted twice more with ethyl acetate. The combined extracts were dried with anhydrous sodium sulfate, filtered and concentrated. The residue was dissolved in dichloromethane and diluted with diethyl ether to precipitate the title compound, which was collected by filtration. MS (ESI) m/e 669.7 (M+H)⁺.

### 2.135.4 (9H-fluoren-9-yl)methyl ((S)-1-(((S)-1-((4-(hydroxymethyl)-3-iodophenyl)amino)-1-oxopropan-2-yl)amino)-3-methyl-1-oxobutan-2-yl)carbamate

Example 2.54.3 (1 g) was dissolved in tetrahydrofuran (15 mL), and the mixture was chilled to −15° C. in an ice-acetone bath. Lithium aluminum hydride (1N in tetrahydrofuran, 3 mL) was then added dropwise, keeping the temperature below −10° C. The reaction was stirred for 1 hour and carefully quenched with 10% citric acid (25 mL). The layers were separated, and the aqueous layer was extracted thrice with ethyl acetate. The combined organic layers were washed with water and brine, dried over anhydrous sodium sulfate, filtered and concentrated. The residue was adsorbed onto silica gel and purified by flash chromatography, eluting with a gradient of 5% to 6% methanol in dichloromethane, to give the title compound. MS (ESI) m/e 664.1 (M+H)⁺.

### 2.135.5 methyl 5-(5-((S)-2-((S)-2-((((9H-fluoren-9-yl)methoxy)carbonyl)amino)-3-methylbutanamido)propanamido)-2-(hydroxymethyl)phenyl)pent-4-ynoate

To a stirred mixture of methyl pent-4-ynoate (50 mg), Example 2.135.4 (180 mg) and N,N-diisopropylethylamine (0.15 mL) in N,N-dimethylformamide (2 mL) was added bis(triphenylphosphine)palladium(II) dichloride (20 mg) and copper iodide (5 mg). The mixture was purged with nitrogen three times and stirred at room temperature overnight. The reaction was diluted with ethyl acetate and washed with water and brine. The aqueous layers were back extracted with ethyl acetate. The combined organic layers were dried over sodium sulfate, filtered and concentrated. The residue was purified by reverse-phase HPLC on a Gilson system, eluting with 20-90% acetonitrile in water containing 0.1% v/v trifluoroacetic acid. The desired fractions were combined and freeze-dried to provide the title compound. MS (ESI) m/e 608.0 (M−H₂O)⁺.

### 2.135.6 methyl 5-(5-((S)-2-((S)-2-((((9H-fluoren-9-yl)methoxy)carbonyl)amino)-3-methylbutanamido)propanamido)-2-(hydroxymethyl)phenyl)pentanoate

A mixture of Example 2.135.5 (0.084 g) and 10% Pd/C (0.02 g) in tetrahydrofuran (5 mL) was stirred at 20° C. under an atmosphere of 50 psi H₂ for 1 hour. The reaction mixture was filtered through diatomaceous earth, and the solvent was evaporated under reduced pressure to provide the title compound. MS (ESI) m/e 612.0 (M−H₂O)⁺.

### 2.135.7 methyl 5-(5-((S)-2-((S)-2-((((9H-fluoren-9-yl)methoxy)carbonyl)amino)-3-methylbutanamido)propanamido)-2-((((4-nitrophenoxy)carbonyl)oxy)methyl)phenyl)pentanoate

Example 2.135.7 was prepared by substituting Example 2.135.7 for Example 2.55.6 in Example 2.55.7. MS (ESI) m/e 795.4 (M+H)⁺.

### 2.135.8 3-(1-((3-(2-((((4-((S)-2-((S)-2-amino-3-methylbutanamido)propanamido)-2-(4-carboxybutyl)benzyl)oxy)carbonyl)(2-sulfoethyl)amino)ethoxy)-5,7-dimethyladamantan-1-yl)methyl)-5-methyl-1H-pyrazol-4-yl)-6-(8-(benzo[d]thiazol-2-ylcarbamoyl)-3,4-dihydroisoquinolin-2(1H)-yl)picolinic Acid

Example 2.135.8 was prepared by substituting 2.135.7 for (9H-fluoren-9-yl)methyl ((S)-3-methyl-1-(((S)-1-((4-((((4-nitrophenoxy)carbonyl)oxy)methyl)phenyl)amino)-1-oxo-5-ureidopentan-2-yl)amino)-1-oxobutan-2-yl)carbamate in Example 2.49.1. MS (ESI) m/e 1271.4 (M−H)⁻.

### 2.135.9 N-[(2,5-dioxo-2,5-dihydro-1H-pyrrol-1-yl)acetyl]-L-valyl-N-{4-[({[2-({3-[(4-{6-[8-(1,3-benzothiazol-2-ylcarbamoyl)-3,4-dihydroisoquinolin-2(1H)-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](2-sulfoethyl)carbamoyl}oxy)methyl]-3-(4-carboxybutyl)phenyl}-L-alaninamide

Example 2.135.9 was prepared by substituting 2.135.8 for Example 2.49.1 in Example 2.54. ¹H NMR (400 MHz, dimethyl sulfoxide-d₆) δ ppm 9.88 (d, 1H), 8.3-8.2 (m, 2H), 8.01 (dd, 1H), 7.77 (d, 1H), 7.59 (dd, 1H), 7.52 (dd, 1H), 7.47-7.29 (m, 8H), 7.23-7.18 (m, 1H), 7.05 (s, 2H), 6.95 (d, 1H), 5.00 (d, 2H), 4.94 (s, 2H), 4.37 (p, 1H), 3.51-3.28 (m, 5H), 3.26-3.14 (m, 2H), 2.99 (t, 2H), 2.65 (t, 2H), 2.57 (s, 2H), 2.26-2.17 (m, 3H), 2.07 (d, 3H), 1.94 (dd, 1H), 1.61-0.69 (m, 35H). MS (ESI) m/e 1408.5 (M−H)⁺.

### 2.136 Synthesis of 2-[({[2-({3-[(4-{6-[8-(1,3-benzothiazol-2-ylcarbamoyl)-3,4-dihydroisoquinolin-2(1H)-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](2-sulfoethyl)carbamoyl}oxy)methyl]-5-(3-{[(2,5-dioxo-2,5-dihydro-1H-pyrrol-1-yl)acetyl]amino}propyl)phenyl Beta-D-Glucopyranosiduronic Acid

### 2.136.1 (3R,4S,5S,6S)-2-(5-(3-((((9H-fluoren-9-yl)methoxy)carbonyl)amino)prop-1-yn-1-yl)-2-formylphenoxy)-6-(methoxycarbonyl)tetrahydro-2H-pyran-3,4,5-triyl Triacetate

Example 2.136.1 was prepared by substituting (9H-fluoren-9-yl)methyl prop-2-yn-1-ylcarbamate for 2.124.1A in Example 2.124.2. MS (ESI) m/e 714.1 (M+H)⁺.

### 2.136.2 (2S,3R,4S,5S,6S)-2-(5-(3-((((9H-fluoren-9-yl)methoxy)carbonyl)amino)propyl)-2-formylphenoxy)-6-(methoxycarbonyl)tetrahydro-2H-pyran-3,4,5-triyl Triacetate

Example 2.136.2 was prepared by substituting 2.136.1 for 2.124.2 in Example 2.124.3. MS (ESI) m/e 718.5 (M+H)⁺.

### 2.136.3 (2S,3R,4S,5S,6S)-2-(5-(3-((((9H-fluoren-9-yl)methoxy)carbonyl)amino)propyl)-2-(hydroxymethyl)phenoxy)-6-(methoxycarbonyl)tetrahydro-2H-pyran-3,4,5-triyl Triacetate

Example 2.136.3 was prepared by substituting 2.136.2 for 2.124.3 in Example 2.124.4. MS (ESI) m/e 742.2 (M+Na)⁺.

### 2.136.4 (2S,3R,4S,5S,6S)-2-(5-(3-((((9H-fluoren-9-yl)methoxy)carbonyl)amino)propyl)-2-((((4-nitrophenoxy)carbonyl)oxy)methyl)phenoxy)-6-(methoxycarbonyl)tetrahydro-2H-pyran-3,4,5-triyl Triacetate

Example 2.136.4 was prepared by substituting 2.136.3 for 2.124.4 in Example 2.124.5. MS (ESI) m/e 885.2 (M+Na)⁺.

### 2.136.5 3-(1-((3-(2-((((4-(3-aminopropyl)-2-(((3R,4S,5S,6S)-6-carboxy-3,4,5-trihydroxytetrahydro-2H-pyran-2-yl)oxy)benzyl)oxy)carbonyl)(2-sulfoethyl)amino)ethoxy)-5,7-dimethyladamantan-1-yl)methyl)-5-methyl-1H-pyrazol-4-yl)-6-(8-(benzo[d]thiazol-2-ylcarbamoyl)-3,4-dihydroisoquinolin-2(1H)-yl)picolinic Acid

Example 2.136.5 was prepared by substituting Example 2.136.4 for (9H-fluoren-9-yl)methyl ((S)-3-methyl-1-(((S)-1-((4-((((4-nitrophenoxy)carbonyl)oxy)methyl)phenyl)amino)-1-oxo-5-ureidopentan-2-yl)amino)-1-oxobutan-2-yl)carbamate in Example 2.49.1. MS (ESI) m/e 1237.7 (M+H)⁺.

### 2.136.6 2-[({[2-({3-[(4-{6-[8-(1,3-benzothiazol-2-ylcarbamoyl)-3,4-dihydroisoquinolin-2(1H)-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](2-sulfoethyl)carbamoyl}oxy)methyl]-5-(3-{[(2,5-dioxo-2,5-dihydro-1H-pyrrol-1-yl)acetyl]amino}propyl)phenyl Beta-D-Glucopyranosiduronic Acid

Example 2.136.6 was prepared by substituting Example 2.136.5 for Example 2.49.1 in Example 2.54. ¹H NMR (400 MHz, dimethyl sulfoxide-d₆) δ ppm 8.14 (d, 1H), 8.01 (d, 1H), 7.59 (d, 1H), 7.53-7.39 (m, 4H), 7.38-7.28 (m, 3H), 7.22-7.15 (m, 2H), 7.13-6.91 (m, 5H), 6.84 (d, 1H), 5.17-4.91 (m, 5H), 3.35-3.2 (m, 4H), 3.10-2.90 (m, 4H), 2.75-2.65 (m, 2H), 2.08 (s, 3H), 1.65 (s, 2H), 1.39-0.71 (m, 21H). MS (ESI) m/e 1372.3 (M−H)⁻.

### 2.137 Synthesis of 6-[8-(1,3-benzothiazol-2-ylcarbamoyl)-3,4-dihydroisoquinolin-2(1H)-yl]-3-{1-[(3-{2-[({[2-{[(2S,3R,4S,5S,6S)-6-carboxy-3,4,5-trihydroxytetrahydro-2H-pyran-2-yl]oxy}-4-(4-{[(2,5-dioxo-2,5-dihydro-1H-pyrrol-1-yl)acetyl]amino}butyl)benzyl]oxy}carbonyl)(3-{[1,3-dihydroxy-2-(hydroxymethyl)propan-2-yl]amino}-3-oxopropyl)amino]ethoxy}-5,7-dimethyltricyclo[3.3.1.1^{3,7}]dec-1-yl)methyl]-5-methyl-1H-pyrazol-4-yl}pyridine-2-carboxylic Acid

### 2.137.1 3-(1-((3-(2-((((4-(4-aminobutyl)-2-(((2R,3S,4R,5R,6R)-6-carboxy-3,4,5-trihydroxytetrahydro-2H-pyran-2-yl)oxy)benzyl)oxy)carbonyl)(3-((1,3-dihydroxy-2-(hydroxymethyl)propan-2-yl)amino)-3-oxopropyl)amino)ethoxy)-5,7-dimethyladamantan-1-yl)methyl)-5-methyl-1H-pyrazol-4-yl)-6-(8-(benzo[d]thiazol-2-ylcarbamoyl)-3,4-dihydroisoquinolin-2(1H)-yl)picolinic Acid

The title compound was prepared as described in Example 2.124.6, replacing Example 1.87.3 with Example 1.84. MS (ESI) m/e 1319.4 (M−H)⁻.

### 2.137.2 6-[8-(1,3-benzothiazol-2-ylcarbamoyl)-3,4-dihydroisoquinolin-2(1H)-yl]-3-{1-[(3-{2-[({[2-{[(2S,3R,4S,5S,6S)-6-carboxy-3,4,5-trihydroxytetrahydro-2H-pyran-2-yl]oxy}-4-(4-{[(2,5-dioxo-2,5-dihydro-1H-pyrrol-1-yl)acetyl]amino}butyl)benzyl]oxy}carbonyl)(3-{[1,3-dihydroxy-2-(hydroxymethyl)propan-2-yl]amino}-3-oxopropyl)amino]ethoxy}-5,7-dimethyltricyclo[3.3.1.1^{3,7}]dec-1-yl)methyl]-5-methyl-1H-pyrazol-4-yl}pyridine-2-carboxylic Acid

The title compound was prepared as described in Example 2.54, replacing Example 2.49.1 with Example 2.137.1. ¹H NMR (501 MHz, dimethyl sulfoxide-d₆) δ ppm 12.83 (s, 2H), 8.12 (s, OH), 8.06 (s, 1H), 8.03-7.99 (m, 1H), 7.77 (d, 1H), 7.72 (s, OH), 7.60 (d, 1H), 7.52-7.39 (m, 3H), 7.34 (td, 2H), 7.26 (s, 1H), 7.21-7.11 (m, 2H), 7.05 (s, 2H), 6.93 (d, 2H), 6.83 (d, 1H), 5.09 (d, 2H), 5.00 (d, 1H), 4.94 (s, 2H), 4.12 (t, 1H), 3.97 (s, 2H), 3.87 (q, 4H), 3.79 (d, 2H), 3.29 (q, 2H), 3.12-2.93 (m, 5H), 2.47-2.23 (m, 1H), 2.07 (d, 3H), 1.50 (d, 3H), 1.36 (d, 5H), 1.31-0.85 (m, 9H), 0.81 (d, 7H). MS (ESI) m/e 1568.4 (M−H)⁻.

### 2.138 Synthesis of 6-(8-(benzo[d]thiazol-2-ylcarbamoyl)naphthalen-2-yl)-3-(1-((3-(2-((((2-(((2S,3R,4S,5S,6S)-6-carboxy-3,4,5-trihydroxytetrahydro-2H-pyran-2-yl)oxy)-4-(4-(2-((3S,5S)-3-(2,5-dioxo-2,5-dihydro-1H-pyrrol-1-yl)-2-oxo-5-((2-sulfoethoxy)methyl)pyrrolidin-1-yl)acetamido)butyl)benzyl)oxy)carbonyl)(2-sulfoethyl)amino)ethoxy)-5,7-dimethyladamantan-1-yl)methyl)-5-methyl-1H-pyrazol-4-yl)picolinic Acid

The title compound was prepared by substituting Example 2.133.1 for Example 2.119.16 in Example 2.119.17. ¹H NMR (400 MHz, dimethyl sulfoxide-d₆) δ ppm 8.99 (s, 1H), 8.34 (dd, 1H), 8.19 (d, 1H), 8.17 (d, 1H), 8.13 (d, 1H), 8.04 (d, 1H), 7.97 (d, 1H), 7.93 (d, 1H), 7.80 (br t, 1H), 7.77 (d, 1H), 7.67 (dd, 1H), 7.45 (s, 1H), 7.45 (dd, 1H), 7.34 (dd, 1H), 7.14 (d, 1H), 7.03 (s, 2H), 6.93 (s, 1H), 6.82 (br d, 1H), 5.06 (br m, 2H), 4.98 (d, 1H), 4.67 (t, 1H), 4.02 (d, 2H), 3.85 (m, 3H), 3.71 (br m, 1H), 3.59 (t, 2H), 3.45 (br m, 3H), 3.41 (m, 4H), 3.27 (m, 4H), 3.03 (m, 2H), 2.70 (m, 2H) 2.65 (br m, 2H), 2.50 (br t, 2H), 2.31 (br m, 1H), 2.19 (s, 3H), 1.80 (m, 1H), 1.52 (br m, 2H), 1.38 (m, 2H), 1.35 (br m, 2H), 1.29-0.88 (br m, 10H), 0.82 (s, 3H), 0.80 (s, 3H). MS (ESI) m/e 1602.4 (M−H)⁻.

### 2.139 Synthesis of 2-[({[2-({3-[(4-{6-[8-(1,3-benzothiazol-2-ylcarbamoyl)-3,4-dihydroisoquinolin-2(1H)-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl][3-hydroxy-2-(hydroxymethyl)propyl]carbamoyl}oxy)methyl]-5-(3-{[(2,5-dioxo-2,5-dihydro-1H-pyrrol-1-yl)acetyl]amino}propyl)phenyl Beta-D-Glucopyranosiduronic Acid

### 2.139.1 3-(1-((3-(2-((((4-(3-aminopropyl)-2-(((2S,3R,4S,5S,6S)-6-carboxy-3,4,5-trihydroxytetrahydro-2H-pyran-2-yl)oxy)benzyl)oxy)carbonyl)(3-hydroxy-2-(hydroxymethyl)propyl)amino)ethoxy)-5,7-dimethyladamantan-1-yl)methyl)-5-methyl-1H-pyrazol-4-yl)-6-(8-(benzo[d]thiazol-2-ylcarbamoyl)-3,4-dihydroisoquinolin-2(1H)-yl)picolinic Acid

Example 2.139.1 was prepared by substituting Example 2.136.4 for (9H-fluoren-9-yl)methyl ((S)-3-methyl-1-(((S)-1-((4-((((4-nitrophenoxy)carbonyl)oxy)methyl)phenyl)amino)-1-oxo-5-ureidopentan-2-yl)amino)-1-oxobutan-2-yl)carbamate and substituting Example 1.79.3 for Example 1.2.9 in Example 2.49.1. MS (ESI) m/e 1217.7 (M+H)⁺.

### 2.139.2 2-[({[2-({3-[(4-{6-[8-(1,3-benzothiazol-2-ylcarbamoyl)-3,4-dihydroisoquinolin-2(1H)-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl][3-hydroxy-2-(hydroxymethyl)propyl]carbamoyl}oxy)methyl]-5-(3-{[(2,5-dioxo-2,5-dihydro-1H-pyrrol-1-yl)acetyl]amino}propyl)phenyl Beta-D-Glucopyranosiduronic Acid

Example 2.139.1 was prepared by substituting Example 2.139.1 for Example 2.49.1 in Example 2.54. ¹H NMR (400 MHz, dimethyl sulfoxide-d₆) δ ppm 12.84 (s, 2H), 8.11 (t, 1H), 8.00 (dd, 1H), 7.76 (d, 1H), 7.62-7.56 (m, 1H), 7.50-7.37 (m, 3H), 7.37-7.29 (m, 2H), 7.25 (s, 1H), 7.16 (d, 1H), 7.04 (s, 2H), 6.96-6.88 (m, 2H), 6.82 (d, 1H), 5.06 (s, 2H), 4.98 (d, 1H), 4.92 (s, 2H), 3.97 (s, 2H), 3.44-3.18 (m, 11H), 3.07-2.90 (m, 4H), 2.05 (s, 3H), 1.80 (s, 1H), 1.64 (p, 2H), 1.38-0.67 (m, 19H). (m, 21H). MS (ESI) m/e 1352.5 (M−H)⁻.

### 2.140 Synthesis of N-({(3S,5S)-3-(2,5-dioxo-2,5-dihydro-1H-pyrrol-1-yl)-2-oxo-5-[(2-sulfoethoxy)methyl]pyrrolidin-1-yl}acetyl)-L-valyl-N-{4-[({[2-({3-[(4-{6-[8-(1,3-benzothiazol-2-ylcarbamoyl)-3,4-dihydroisoquinolin-2(1H)-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](2-sulfoethyl)carbamoyl}oxy)methyl]-3-(2,5,8,11,14,17,20,23,26,29,32,35,38,41,44,47,50-heptadecaoxatripentacont-52-yn-53-yl)phenyl}-L-alaninamide

### 2.140.1 2-iodo-4-nitrobenzoic Acid

2-Amino-4-nitrobenzoic acid (50 g) was added to a mixture of concentrated H₂SO₄ (75 mL) and water (750 mL) at 0° C., and the mixture was stirred for 1 hour. To the mixture was added a mixture of sodium nitrite (24.62 g) in water (300 mL) dropwise at 0° C. The resulting mixture was stirred at 0° C. for 3 hours. A mixture of sodium iodide (65.8 g) in water (300 mL) was added to above mixture slowly. After the completion of the addition, the resulting mixture was stirred at 0° C. for 2 hours, then at room temperature for 16 hours and at 60° C. for 2 hours. The resulting mixture was cooled to room temperature and diluted with ice-water (300 mL). The solid was collected by filtration, washed by water (100 mL×5), and dried in air for 16 hours to give the title compound. MS (LC-MS) m/e 291.9 (M−H)⁻.

### 2.140.2 methyl 2-iodo-4-nitrobenzoate

A mixture of Example 2.140.1 (130 g) in a mixture of methanol (1000 mL) and sulfuric acid (23.65 mL) was stirred at 85° C. for 16 hours and concentrated to dryness. The residue was triturated with methanol (100 mL) and the suspension was stirred for 10 minutes. The solid was collected by filtration, washed with water (200 mL×3) and methanol (20 mL), and air-dried for 16 hours to give the title compound. MS (LC-MS) m/e 308.0 (M+H)⁺.

### 2.140.3 methyl 4-amino-2-iodobenzoate

To a mixture of ammonium chloride (122 g) and iron (38.2 g) in ethanol (1000 mL) and water (100 mL) was added Example 2.140.2 (70 g,) at room temperature. The mixture was stirred at 80° C. for 4 hours and filtered to remove insoluble material. The filtrate was concentrated under reduced pressure. The residue was dissolved in ethyl acetate (1000 mL) and washed with water (500 mL). The aqueous phase was extracted with ethyl acetate (1000 mL×2). The combined organic phase was washed with brine, dried over MgSO₄, filtered and concentrated to give the title compound. MS (LC-MS) m/e 278.0 (M+H)⁺.

### 2.140.4 (4-amino-2-iodophenyl)methanol

To a mixture of Example 2.140.3 (40 g) in tetrahydrofuran (800 mL) was added 1M diisobutylaluminum hydride (505 mL) dropwise at −50° C. The mixture was stirred at −50° C. for 3 hours and cooled to −20° C. Ice-water (180 mL) was added dropwise (keeping temperature below 0° C.) to the mixture. After the addition of ice-water, the mixture was stirred for 10 minutes and filtered. The filtrate was concentrated, and the residue was dissolved in ethyl acetate (800 mL) and water (200 mL). The aqueous phase was extracted with ethyl acetate (300 mL×2). The combined organic phases were washed with brine, dried over MgSO₄, filtered and concentrated to give the title compound. MS (LC-MS) m/e 250.0 (M+H)⁺.

### 2.140.5 4-(((tert-butyldimethylsilyl)oxy)methyl)-3-iodoaniline

To a mixture of Example 2.140.4 (40 g) and imidazole (21.87 g) in dichloromethane (600 mL) and tetrahydrofuran (150 mL) was added tert-butyldimethylchlorosilane (29.0 g). The mixture was stirred at room temperature for 16 hours and filtered to remove the solid. To the filtrate was added ice-water (50 mL). The mixture was stirred for 10 minutes and water (100 mL) was added. The mixture was extracted with dichloromethane (500 mL×2). The combined organic phases were washed with brine, dried over MgSO₄, filtered and concentrated. The residue was purified by silica gel chromatography, eluting with 15/1 to 10/1 petroleum ether/ethyl acetate, to give the title compound. MS (LC-MS) m/e 364.0 (M+H)⁺.

### 2.140.6 (S)-tert-butyl (1-((4-(((tert-butyldimethylsilyl)oxy)methyl)-3-iodophenyl)amino)-1-oxopropan-2-yl)carbamate

To a mixed mixture of (S)-2-((tert-butoxycarbonyl)amino)propanoic acid (15.62 g) and Example 2.140.5 (30 g) in dichloromethane (600 mL) at 0° C. was added POCl₃ (15.39 mL) dropwise. The mixture was stirred at 0° C. for 2 hours. Ice-water (60 mL) was carefully added to the mixture dropwise (keeping temperature below 5° C.). The mixture was stirred for 30 minutes and concentrated to remove dichloromethane. The residue was suspended in ethyl acetate (500 mL) and water (100 mL). The suspension was filtered. The organic phase was washed by water (200 mL×2) and brine, dried over MgSO₄, filtered and concentrated to give the title compound. MS (LC-MS) m/e 533.0 (M−H)⁺.

### 2.140.7 (S)-tert-butyl (1-((4-(hydroxymethyl)-3-iodophenyl)amino)-1-oxopropan-2-yl)carbamate

To a mixture of Example 2.140.6 (60 g) in tetrahydrofuran (600 mL) was added tetrabutyl ammonium fluoride (28.2 g) in tetrahydrofuran (120 mL) at 0° C. The mixture was stirred at room temperature for 16 hours and filtered. To the filtrate was added water (100 mL). The mixture was stirred for 10 minutes and then concentrated. The residue was diluted with ethyl acetate (800 mL) and water (300 mL). The aqueous phase was extracted with ethyl acetate (200 mL×3). The combined organic phases were washed with brine, dried over MgSO₄, filtered and concentrated. The residue was purified by silica gel chromatography, eluting with 3/1 to 1/1 petroleum ether/ethyl acetate, to give the title compound. MS (LC-MS) m/e 443.0 (M+Na)⁺.

### 2.140.8 (S)-2-amino-N-(4-(hydroxymethyl)-3-iodophenyl)propanamide

A mixture of Example 2.140.7 (20 g) in a mixture of dichloromethane (80 mL) and trifluoroacetic acid (40 mL) was stirred at room temperature for 2 hours and concentrated. The residue was dissolved in dichloromethane (80 mL) and triethylamine (16.95 mL) was added to adjust the pH to 8. The title compound was obtained as free base in dichloromethane, which was used in next step without further purification. MS (LC-MS) m/e 321.1 (M+H)⁺.

### 2.140.9 tert-butyl ((S)-1-(((S)-1-((4-(hydroxymethyl)-3-iodophenyl)amino)-1-oxopropan-2-yl)amino)-3-methyl-1-oxobutan-2-yl)carbamate

A mixture of (S)-2-((tert-butoxycarbonyl)amino)-3-methylbutanoic acid (6.79 g), triethylamine (9.58 mL) and 1-hydroxybenzotriazole hydrate (5.26 g) in dichloromethane (250 mL) was stirred for 20 minutes. The resulting mixture was added to a mixture of Example 2.140.8 (10 g) and 1-ethyl-3-[3-(dimethylamino)propyl]-carbodiimide hydrochloride (6.59 g) in dichloromethane (100 mL) at 0° C., dropwise. After the completion of addition, the mixture was stirred at 0° C. for 2 hours. Ice-water (200 mL) was added, and the resulting mixture was stirred for 20 minutes. The organic phase was washed with saturated aqueous sodium bicarbonate mixture (100 mL×2), water (100 mL×2) and brine (100 mL), dried over MgSO₄, filtered and concentrated. The residue was purified by silica gel chromatography, eluting with 3/1 to 1/1 petroleum ether/ethyl acetate, to give the title compound. LC-MS m/e 542.1 (M+Na)⁺.

### 2.140.10 tert-butyl ((S)-1-(((S)-1-((4-(hydroxymethyl)-3-(2,5,8,11,14,17,20,23,26,29,32,35,38,41,44,47,50-heptadecaoxatripentacont-52-yn-53-yl)phenyl)amino)-1-oxopropan-2-yl)amino)-3-methyl-1-oxobutan-2-yl)carbamate

To a mixture of Example 2.140.9 (50 mg), 2,5,8,11,14,17,20,23,26,29,32,35,38,41,44,47,50-heptadecaoxatripentacont-52-yne (149 mg), bis(triphenylphosphine)palladium(II) dichloride (27.0 mg) and N,N-diisopropylethylamine (0.05 mL) in N,N-dimethylformamide (1 mL) was added copper(I) iodide (3.67 mg). The reaction was purged with a stream of nitrogen gas for 10 minutes and stirred overnight. The reaction was diluted with dimethyl sulfoxide purified by reverse-phase HPLC on a Gilson system (C18 column), eluting with 20-70% acetonitrile in water containing 0.1% trifluoroacetic acid, to give the title compound. MS (LC-MS) m/e 1164.2 (M−H)⁻.

### 2.140.11 tert-butyl ((S)-3-methyl-1-(((S)-1-((4-((((4-nitrophenoxy)carbonyl)oxy)methyl)-3-(2,5,8,11,14,17,20,23,26,29,32,35,38,41,44,47,50-heptadecaoxatripentacont-52-yn-53-yl)phenyl)amino)-1-oxopropan-2-yl)amino)-1-oxobutan-2-yl)carbamate

To a mixture of Example 2.140.10 (80 mg) and bis(4-nitrophenyl) carbonate (31.3 mg) in N,N-dimethylformamide (0.2 mL) was added N,N-diisopropylethylamine (0.06 mL). The mixture was stirred 3 hours and was purified by reverse-phase HPLC on a Gilson system (C18 column), eluting with 35-75% acetonitrile in water containing 0.1% trifluoroacetic acid, to give the title compound.

### 2.140.12 6-(8-(benzo[d]thiazol-2-ylcarbamoyl)-3,4-dihydroisoquinolin-2(1H)-yl)-3-(1-((3-(2-((((4-((S)-2-((S)-2-((tert-butoxycarbonyl)amino)-3-methylbutanamido)propanamido)-2-(2,5,8,11,14,17,20,23,26,29,32,35,38,41,44,47,50-heptadecaoxatripentacont-52-yn-53-yl)benzyl)oxy)carbonyl)(2-sulfoethyl)amino)ethoxy)-5,7-dimethyladamantan-1-yl)methyl)-5-methyl-1H-pyrazol-4-yl)picolinic Acid

To a mixture of Example 1.2.9 (95 mg), Example 2.140.11 (148 mg) and 1-hydroxybenzotriazole hydrate (68.1 mg) in N,N-dimethylformamide (2.5 mL) was added N,N-diisopropylethylamine (97 μL). The mixture was stirred for 3.5 hours and purified by reverse-phase HPLC on a Gilson system (C18 column), eluting with 35-80% acetonitrile in water containing 0.1% trifluoroacetic acid, to give the title compound.

### 2.140.13 3-(1-((3-(2-((((4-((S)-2-((S)-2-amino-3-methylbutanamido)propanamido)-2-(2,5,8,11,14,17,20,23,26,29,32,35,38,41,44,47,50-heptadecaoxatripentacont-52-yn-53-yl)benzyl)oxy)carbonyl)(2-sulfoethyl)amino)ethoxy)-5,7-dimethyladamantan-1-yl)methyl)-5-methyl-1H-pyrazol-4-yl)-6-(8-(benzo[d]thiazol-2-ylcarbamoyl)-3,4-dihydroisoquinolin-2(1H)-yl)picolinic Acid

A cold (0° C.) mixture of Example 2.140.12 (135 mg) in dichloromethane (4 mL) was treated with trifluoroacetic acid (1 mL) for 5 hours. The mixture was concentrated and purified by reverse-phase HPLC on a Gilson system (C18 column), eluting with 20-60% acetonitrile in water containing 0.1% trifluoroacetic acid, to give the title compound. MS (ESI) m/e 973.4 (M+2H)²⁺.

### 2.140.14 N-({(3S,5S)-3-(2,5-dioxo-2,5-dihydro-1H-pyrrol-1-yl)-2-oxo-5-[(2-sulfoethoxy)methyl]pyrrolidin-1-yl}acetyl)-L-valyl-N-{4-[({[2-({3-[(4-{6-[8-(1,3-benzothiazol-2-ylcarbamoyl)-3,4-dihydroisoquinolin-2(1H)-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](2-sulfoethyl)carbamoyl}oxy)methyl]-3-(2,5,8,11,14,17,20,23,26,29,32,35,38,41,44,47,50-heptadecaoxatripentacont-52-yn-53-yl)phenyl}-L-alaninamide

A mixture of Example 2.119.15 (20.88 mg) and O-(7-azabenzotriazol-1-yl)-N,N,N′,N′-tetramethyluronium hexafluorophosphate (21.1 mg) in N,N-dimethylformamide (0.4 mL) was treated with N,N-diisopropylethylamine (16.2 μL) for 7 minutes, and a mixture of Example 2.140.13 (60 mg) and N,N-diisopropylethylamine (32.3 μL) in N,N-dimethylformamide (0.6 mL) was slowly added. The reaction mixture was stirred for 10 minutes and was purified by reverse-phase HPLC on a Gilson system (C18 column), eluting with 20-70% acetonitrile in water containing 0.1% trifluoroacetic acid, to give the title compound. ¹H NMR (500 MHz, dimethyl sulfoxide-d₆) δ 10.01 (d, 1H), 8.22 (d, 1H), 8.02 (t, 2H), 7.90-7.75 (m, 2H), 7.66-7.50 (m, 3H), 7.50-7.39 (m, 3H), 7.35 (q, 3H), 7.05 (s, 2H), 7.00 (d, 1H), 5.08 (d, 2H), 4.97 (s, 2H), 4.65 (t, 1H), 4.47-4.31 (m, 4H), 4.23-4.14 (m, 2H), 3.90-3.69 (m, 5H), 3.68-3.58 (m, 4H), 3.57-3.53 (m, 2H), 3.52-3.43 (m, 57H), 3.42-3.33 (m, 4H), 3.22 (s, 5H), 3.01 (t, 2H), 2.49 (p, 3H), 2.09 (d, 3H), 2.04-1.77 (m, 1H), 1.40-1.17 (m, 6H), 1.06 (dd, 6H), 0.97-0.63 (m, 11H). MS (ESI) m/e 1153.3 (M+2H)²⁺.

### 2.141 Synthesis of N-({(3S,5S)-3-(2,5-dioxo-2,5-dihydro-1H-pyrrol-1-yl)-2-oxo-5-[(2-sulfoethoxy)methyl]pyrrolidin-1-yl}acetyl)-L-valyl-N-{4-[({[2-({3-[(4-{6-[8-(1,3-benzothiazol-2-ylcarbamoyl)-3,4-dihydroisoquinolin-2(1H)-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](2-sulfoethyl)carbamoyl}oxy)methyl]-3-(2,5,8,11,14,17,20,23,26,29,32,35,38,41,44,47,50-heptadecaoxatripentacontan-53-yl)phenyl}-L-alaninamide

### 2.141.1 tert-butyl ((S)-1-(((S)-1-((3-(2,5,8,11,14,17,20,23,26,29,32,35,38,41,44,47,50-heptadecaoxadopentacontan-52-yl)-4-(hydroxymethyl)phenyl)amino)-1-oxopropan-2-yl)amino)-3-methyl-1-oxobutan-2-yl)carbamate

A mixture of Example 2.140.10 (304 mg) and 10% Pd/C (90 mg, dry) in tetrahydrofuran (20 mL) was shaken in a pressure bottle for 2 hours under 50 psi of hydrogen gas. The insoluble material was filtered off, and the filtrate was concentrated to provide the title compound. MS (ESI) m/e 1168.3 (M−H)⁻.

### 2.141.2 tert-butyl ((S)-1-(((S)-1-((3-(2,5,8,11,14,17,20,23,26,29,32,35,38,41,44,47,50-heptadecaoxadopentacontan-52-yl)-4-((((4-nitrophenoxy)carbonyl)oxy)methyl)phenyl)amino)-1-oxopropan-2-yl)amino)-3-methyl-1-oxobutan-2-yl)carbamate

The title compound was prepared using the procedure in Example 2.140.11, replacing Example 2.140.10 with Example 2.141.1.

### 2.141.3 6-(8-(benzo[d]thiazol-2-ylcarbamoyl)-3,4-dihydroisoquinolin-2(1H)-yl)-3-(1-((3-(2-((((4-((S)-2-((S)-2-((tert-butoxycarbonyl)amino)-3-methylbutanamido)propanamido)-2-(2,5,8,11,14,17,20,23,26,29,32,35,38,41,44,47,50-heptadecaoxatripentacontan-53-yl)benzyl)oxy)carbonyl)(2-sulfoethyl)amino)ethoxy)-5,7-dimethyladamantan-1-yl)methyl)-5-methyl-1H-pyrazol-4-yl)picolinic Acid

The title compound was prepared using the procedure in Example 2.140.12, replacing Example 2.140.11 with Example 2.141.2.

### 2.141.4 3-(1-((3-(2-((((4-((S)-2-((S)-2-amino-3-methylbutanamido)propanamido)-2-(2,5,8,11,14,17,20,23,26,29,32,35,38,41,44,47,50-heptadecaoxatripentacontan-53-yl)benzyl)oxy)carbonyl)(2-sulfoethyl)amino)ethoxy)-5,7-dimethyladamantan-1-yl)methyl)-5-methyl-1H-pyrazol-4-yl)-6-(8-(benzo[d]thiazol-2-ylcarbamoyl)-3,4-dihydroisoquinolin-2(1H)-yl)picolinic Acid

The title compound was prepared using the procedure in Example 2.140.13, replacing Example 2.140.12 with Example 2.141.3. MS (ESI) m/e 1948.8 (M−H)⁻.

### 2.141.5 N-({(3S,5S)-3-(2,5-dioxo-2,5-dihydro-1H-pyrrol-1-yl)-2-oxo-5-[(2-sulfoethoxy)methyl]pyrrolidin-1-yl}acetyl)-L-valyl-N-{4-[({[2-({3-[(4-{6-[8-(1,3-benzothiazol-2-ylcarbamoyl)-3,4-dihydroisoquinolin-2(1H)-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](2-sulfoethyl)carbamoyl}oxy)methyl]-3-(2,5,8,11,14,17,20,23,26,29,32,35,38,41,44,47,50-heptadecaoxatripentacontan-53-yl)phenyl}-L-alaninamide

The title compound was prepared using the procedure in Example 2.140.14, replacing Example 2.140.13 with Example 2.141.4. ¹H NMR (501 MHz, dimethyl sulfoxide-d₆) δ 12.87 (s, 1H), 9.84 (s, 1H), 8.18 (d, 1H), 8.03 (dd, 2H), 7.78 (d, 1H), 7.61 (d, 1H), 7.52 (d, 1H), 7.45 (ddd, 4H), 7.40-7.32 (m, 2H), 7.30 (s, 1H), 7.22 (d, 1H), 7.07 (s, 2H), 6.96 (d, 1H), 5.01 (d, 2H), 4.95 (s, 2H), 4.64 (t, 1H), 4.38 (t, 1H), 4.24-4.12 (m, 2H), 4.00 (d, 1H), 3.88 (t, 2H), 3.78 (t, 3H), 3.64 (ddt, 2H), 3.49 (dd, 62H), 3.43-3.37 (m, 6H), 3.23 (s, 3H), 3.01 (t, 2H), 2.84-2.68 (m, 1.5H), 2.63 (dd, 4H), 2.36 (d, 0.5H), 2.08 (d, 3H), 1.74 (t, 2H), 1.25 (dt, 6H), 1.17-1.00 (m, 6H), 0.99-0.72 (m, 11H). MS (ESI) m/e 1153.0 (M−2H)²⁻.

### 2.141 Synthesis of 2-[({[2-({3-[(4-{6-[8-(1,3-benzothiazol-2-ylcarbamoyl)-3,4-dihydroisoquinolin-2(1H)-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl][(3S)-3,4-dihydroxybutyl]carbamoyl}oxy)methyl]-5-(3-{[(2,5-dioxo-2,5-dihydro-1H-pyrrol-1-yl)acetyl]amino}propyl)phenyl Beta-D-Glucopyranosiduronic Acid

### 2.142.1 3-(1-((3-(2-((((4-(3-aminopropyl)-2-(((2S,3R,4S,5S,6S)-6-carboxy-3,4,5-trihydroxytetrahydro-2H-pyran-2-yl)oxy)benzyl)oxy)carbonyl)((S)-3,4-dihydroxybutyl)amino)ethoxy)-5,7-dimethyladamantan-1-yl)methyl)-5-methyl-1H-pyrazol-4-yl)-6-(8-(benzo[d]thiazol-2-ylcarbamoyl)-3,4-dihydroisoquinolin-2(1H)-yl)picolinic Acid

Example 2.142.1 was prepared by substituting Example 2.136.4 for (9H-fluoren-9-yl)methyl ((S)-3-methyl-1-(((S)-1-((4-((((4-nitrophenoxy)carbonyl)oxy)methyl)phenyl)amino)-1-oxo-5-ureidopentan-2-yl)amino)-1-oxobutan-2-yl)carbamate and substituting Example 1.85 for Example 1.2.9 in Example 2.49.1. MS (ESI) m/e 1217.3 (M+H)⁺.

### 2.142.2 2-[({[2-({3-[(4-{6-[8-(1,3-benzothiazol-2-ylcarbamoyl)-3,4-dihydroisoquinolin-2(1H)-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl][(3S)-3,4-dihydroxybutyl]carbamoyl}oxy)methyl]-5-(3-{[(2,5-dioxo-2,5-dihydro-1H-pyrrol-1-yl)acetyl]amino}propyl)phenyl Beta-D-Glucopyranosiduronic Acid

Example 2.142.2 was prepared by substituting Example 2.142.1 for Example 2.49.1 in Example 2.54. ¹H NMR (400 MHz, dimethyl sulfoxide-d₆) δ ppm 8.14 (d, 1H), 8.03 (dt, 1H), 7.81-7.76 (m, 1H), 7.61 (dd, 1H), 7.53-7.41 (m, 3H), 7.38-7.32 (m, 2H), 7.28 (s, 1H), 7.18 (d, 1H), 7.06 (d, 2H), 6.97-6.92 (m, 2H), 6.85 (dd, 1H), 5.10 (q, 2H), 5.01 (d, 1H), 4.96 (s, 2H), 3.48-3.18 (m, 12H), 3.06 (q, 2H), 3.00 (t, 2H), 2.08 (s, 3H), 1.77-0.66 (m, 16H). MS (ESI) m/e 1352.5 (M−H)⁻.

### 2.143 Synthesis of 1-{[2-({3-[(4-{6-[8-(1,3-benzothiazol-2-ylcarbamoyl)-3,4-dihydroisoquinolin-2(1H)-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl]({[4-(4-{[(2,5-dioxo-2,5-dihydro-1H-pyrrol-1-yl)acetyl]amino}butyl)-2-(beta-D-glucopyranuronosyloxy)benzyl]oxy}carbonyl)amino}-1,2-dideoxy-D-arabino-hexitol

### 2.143.1 3-(1-((3-(2-((((4-(4-aminobutyl)-2-(((2S,3R,4S,5S,6S)-6-carboxy-3,4,5-trihydroxytetrahydro-2H-pyran-2-yl)oxy)benzyl)oxy)carbonyl)((3R,4S,5R)-3,4,5,6-tetrahydroxyhexyl)amino)ethoxy)-5,7-dimethyladamantan-1-yl)methyl)-5-methyl-1H-pyrazol-4-yl)-6-(8-(benzo[d]thiazol-2-ylcarbamoyl)-3,4-dihydroisoquinolin-2(1H)-yl)picolinic Acid

The title compound was prepared by substituting Example 1.77.2 for Example 1.25 and Example 2.124.5 for Example 2.97.7 in Example 2.97.8. MS (ESI) m/e 1291 (M+H)⁺, 1289 (M−H)⁻.

### 2.143.2 1-{[2-({3-[(4-{6-[8-(1,3-benzothiazol-2-ylcarbamoyl)-3,4-dihydroisoquinolin-2(1H)-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl]({[4-(4-{[(2,5-dioxo-2,5-dihydro-1H-pyrrol-1-yl)acetyl]amino}butyl)-2-(beta-D-glucopyranuronosyloxy)benzyl]oxy}carbonyl)amino}-1,2-dideoxy-D-arabino-hexitol

The title compound was prepared by substituting Example 2.143.1 for Example 2.49.1 in Example 2.54. ¹H NMR (400 MHz, dimethyl sulfoxide-d₆) δ ppm 8.04 (d, 1H), 7.81 (d, 1H), 7.61 (d, 1H), 7.54-7.43 (m, 3H), 7.41-7.35 (m, 2H), 7.29 (s, 1H), 7.18 (m, 1H), 7.03 (s, 2H), 6.97 (d, 1H), 6.93 (s, 1H), 6.86 (d, 1H), 5.18-5.05 (m, 3H), 5.03 (d, 1H), 4.97 (s, 2H), 4.01 (s, 2H), 3.91 (d, 1H), 3.87 (t, 2H), 3.83 (m, 2H), 3.72 (s, 2H), 3.67 (m, 2H), 3.59 (dd, 2H), 3.50-3.27 (m, 16H), 3.14 (d, 2H), 3.04 (m, 4H), 2.09 (s, 3H), 1.68 (m, 2H), 1.52 (m, 2H), 1.44-1.31 (m, 4H), 1.26-1.14 (m, 4H), 1.10 (m, 4H), 0.98 (q, 2H), 0.85 (m, 6H). MS (ESI) m/e 1428 (M+H)⁺, 1426 (M−H)⁻.

### 2.144 Synthesis of 1-{[2-({3-[(4-{6-[8-(1,3-benzothiazol-2-ylcarbamoyl)-3,4-dihydroisoquinolin-2(1H)-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl]({[4-(4-{[(2,5-dioxo-2,5-dihydro-1H-pyrrol-1-yl)acetyl]amino}butyl)-2-(beta-D-glucopyranuronosyloxy)benzyl]oxy}carbonyl)amino}-1,2-dideoxy-D-erythro-pentitol

### 2.144.1 3-(1-((3-(2-((((4-(4-aminobutyl)-2-(((2S,3R,4S,5S,6S)-6-carboxy-3,4,5-trihydroxytetrahydro-2H-pyran-2-yl)oxy)benzyl)oxy)carbonyl)((3S,4R)-3,4,5-trihydroxypentyl)amino)ethoxy)-5,7-dimethyladamantan-1-yl)methyl)-5-methyl-1H-pyrazol-4-yl)-6-(8-(benzo[d]thiazol-2-ylcarbamoyl)-3,4-dihydroisoquinolin-2(1H)-yl)picolinic Acid

The title compound was prepared by substituting Example 1.80 for Example 1.25 and Example 2.124.5 for Example 2.97.7 in Example 2.97.8. MS (ESI) m/e 1261 (M+H)⁺, 1259 (M−H)⁻.

### 2.144.2 1-{[2-({3-[(4-{6-[8-(1,3-benzothiazol-2-ylcarbamoyl)-3,4-dihydroisoquinolin-2(1H)-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl]({[4-(4-{[(2,5-dioxo-2,5-dihydro-1H-pyrrol-1-yl)acetyl]amino}butyl)-2-(beta-D-glucopyranuronosyloxy)benzyl]oxy}carbonyl)amino}-1,2-dideoxy-D-erythro-pentitol

The title compound was prepared by substituting Example 2.144.1 for Example 2.49.1 in Example 2.54. ¹H NMR (400 MHz, dimethyl sulfoxide-d₆) δ ppm 8.08 (t, 1H), 8.03 (d, 1H), 7.79 (d, 1H), 7.62 (d, 1H), 7.53-7.42 (m, 3H), 7.38-7.33 (m, 2H), 7.20 (s, 1H), 7.17 (m, 1H), 7.07 (s, 2H), 6.97-6.93 (m, 2H), 6.85 (d, 1H), 5.17-5.05 (m, 3H), 5.02 (d, 1H), 4.96 (s, 2H), 3.98 (s, 2H), 3.88 (m, 4H), 3.80 (m, 4H), 3.67 (m, 2H), 3.42 (m, 4H), 3.36-3.23 (m, 13H), 3.08-2.99 (m, 5H), 2.09 (s, 3H), 1.86 (m, 1H), 1.53 (m, 2H), 1.38 (m, 4H), 1.25 (m, 4H), 1.11 (m, 4H), 0.96 (m, 2H), 0.83 (m, 6H). MS (ESI) m/e 1398 (M+H)⁺, 1396 (M−H)⁻.

### 2.145 Synthesis of N-[(2,5-dioxo-2,5-dihydro-1H-pyrrol-1-yl)acetyl]-L-valyl-N-{4-[({[2-({3-[(4-{6-[8-(1,3-benzothiazol-2-ylcarbamoyl)naphthalen-2-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](2-sulfoethyl)carbamoyl}oxy)methyl]-3-[27-(2,5,8,11,14,17,20,23-octaoxahexacosan-26-yl)-2,5,8,11,14,17,20,23-octaoxa-27-azatriacontan-30-yl]phenyl}-L-alaninamide

### 2.145.1 tert-butyl ((S)-1-(((S)-1-((3-(3-(((benzyloxy)carbonyl)amino)prop-1-yn-1-yl)-4-(hydroxymethyl)phenyl)amino)-1-oxopropan-2-yl)amino)-3-methyl-1-oxobutan-2-yl)carbamate

To a mixture of tert-butyl ((S)-1-(((S)-1-((4-(hydroxymethyl)-3-iodophenyl)amino)-1-oxopropan-2-yl)amino)-3-methyl-1-oxobutan-2-yl)carbamate (0.5 g) in N,N-dimethylformamide (6 mL) was added benzyl prop-2-yn-1-ylcarbamate (0.182 g), CuI (9.2 mg), bis(triphenylphosphine)palladium(II) dichloride (35 mg) and N,N-diisopropylethylamine (1.0 mL). The mixture was stirred at room temperature overnight. The mixture was concentrated under vacuum. The residue was dissolved in ethyl acetate (300 mL), washed with water, brine, dried over anhydrous sodium sulfate, filtered and concentrated. Evaporation of the solvent, and purification of the residue by silica gel chromatography, eluting with 30% ethyl acetate in dichloromethane, gave the title compound. MS (APCI) m/e 581.2 (M−H)⁻.

### 2.145.2 tert-butyl ((S)-1-(((S)-1-((3-(3-aminopropyl)-4-(hydroxymethyl)phenyl)amino)-1-oxopropan-2-yl)amino)-3-methyl-1-oxobutan-2-yl)carbamate

To a mixture of Example 2.145.1 (1.7 g) in ethanol (30 mL) was added 5% Pd/C (0.3 g) and cyclohexene (large excess). The reaction was stirred at 100° C. for 45 minutes. The reaction was filtered and concentrated under reduced pressure. The residue was dissolved in N,N-dimethylformamide and purified by reverse-phase HPLC on a Gilson system (C18 column), eluting with 20-80% acetonitrile in water containing 0.1% trifluoroacetic acid, to give the title compound. MS (ESI) m/e 451.1 (M−H)⁻.

### 2.145.3 tert-butyl ((S)-1-(((S)-1-((3-(27-(2,5,8,11,14,17,20,23-octaoxahexacosan-26-yl)-2,5,8,11,14,17,20,23-octaoxa-27-azatriacontan-30-yl)-4-(hydroxymethyl)phenyl)amino)-1-oxopropan-2-yl)amino)-3-methyl-1-oxobutan-2-yl)carbamate

To a mixture of Example 2.145.2 (45 mg) in dichloromethane (4 mL) was added 2,5,8,11,14,17,20,23-octaoxahexacosan-26-al (79 mg) followed by NaH(OAc)₃ (63.5 mg). The mixture was stirred at room temperature for 3 hours and then concentrated under reduced pressure. The residue was dissolved in N,N-dimethylformamide and purified by reverse-phase HPLC on a Gilson system (C18 column), eluting with 20-80% acetonitrile in water containing 0.1% trifluoroacetic acid, to give the title compound. MS (ESI) m/e 1212.1 (M−H)⁻.

### 2.145.4 tert-butyl ((S)-1-(((S)-1-((3-(27-(2,5,8,11,14,17,20,23-octaoxahexacosan-26-yl)-2,5,8,11,14,17,20,23-octaoxa-27-azatriacontan-30-yl)-4-((((4-nitrophenoxy)carbonyl)oxy)methyl)phenyl)amino)-1-oxopropan-2-yl)amino)-3-methyl-1-oxobutan-2-yl)carbamate

To a mixture of Example 2.145.3 (80 mg) in N,N-dimethylformamide (2 mL) was added bis(4-nitrophenyl) carbonate (26 mg) followed by N,N-diisopropylamine (0.012 mL). The mixture was stirred at room temperature overnight and purified directly by reverse phase HPLC on a Gilson system (C18 column), eluting with 20-80% acetonitrile in water containing 0.1% trifluoroacetic acid, to give the title compound. MS (ESI) m/e 1376.97 (M−H)⁻.

### 2.145.5 3-(1-((3-(2-((((2-(27-(2,5,8,11,14,17,20,23-octaoxahexacosan-26-yl)-2,5,8,11,14,17,20,23-octaoxa-27-azatriacontan-30-yl)-4-((S)-2-((S)-2-amino-3-methylbutanamido)propanamido)benzyl)oxy)carbonyl)(2-sulfoethyl)amino)ethoxy)-5,7-dimethyladamantan-1-yl)methyl)-5-methyl-1H-pyrazol-4-yl)-6-(8-(benzo[d]thiazol-2-ylcarbamoyl)naphthalen-2-yl)picolinic Acid

To a mixture of Example 2.145.4 (30 mg) in N,N-dimethylformamide (4 mL) was added Example 1.43 (18.68 mg) followed by 1-hydroxybenzotriazole hydrate (3.4 mg) and N,N-diisopropylamine (3.84 uL). The mixture was stirred at room temperature overnight. Trifluoroacetic acid (0.55 mL) was added to the mixture and stirred at room temperature for 3 hours. The mixture was purified by reverse-phase HPLC on a Gilson system (C18 column), eluting with 20-80% acetonitrile in water containing 0.1% trifluoroacetic acid, to give the title compound. MS (ESI) m/e 1986.6 (M−H)⁻.

### 2.145.6 N-[(2,5-dioxo-2,5-dihydro-1H-pyrrol-1-yl)acetyl]-L-valyl-N-{4-[({[2-({3-[(4-{6-[8-(1,3-benzothiazol-2-ylcarbamoyl)naphthalen-2-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](2-sulfoethyl)carbamoyl}oxy)methyl]-3-[27-(2,5,8,11,14,17,20,23-octaoxahexacosan-26-yl)-2,5,8,11,14,17,20,23-octaoxa-27-azatriacontan-30-yl]phenyl}-L-alaninamide

The title compound was prepared as described in Example 2.123.21, replacing Example 2.123.20 with Example 2.145.5. ¹H NMR (400 MHz, dimethyl sulfoxide-d₆) δ ppm 13.10 (s, 1H), 9.92 (s, 1H), 9.43 (s, 1H), 9.02 (s, 1H), 8.37 (dd, 1H), 8.30-8.14 (m, 5H), 8.07 (d, 1H), 8.02 (d, 1H), 7.96 (d, 1H), 7.81 (d, 1H), 7.74-7.68 (m, 1H), 7.57 (s, 1H), 7.52-7.45 (m, 2H), 7.42-7.34 (m, 2H), 7.28 (d, 1H), 7.08 (s, 2H), 5.05 (d, 2H), 4.39 (t, 1H), 4.21 (dd, 1H), 4.12 (s, 2H), 3.88 (s, 2H), 3.49 (d, 55H), 3.34 (s, 200H), 3.23 (s, 5H), 3.13 (d, 4H), 2.79-2.65 (m, 5H), 2.23 (s, 3H), 1.94 (d, 8H), 1.47-0.94 (m, 15H), 0.92-0.76 (m, 12H).

### 2.146 Synthesis of (6S)-2,6-anhydro-6-(2-{2-[({[2-({3-[(4-{6-[8-(1,3-benzothiazol-2-ylcarbamoyl)-3,4-dihydroisoquinolin-2(1H)-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](2-sulfoethyl)carbamoyl}oxy)methyl]-5-({N-[(2S)-3-[3,4-bis(2,5,8,11,14,17,20,23,26,29,32-undecaoxatetratriacontan-34-yloxy)phenyl]-2-(2,5-dioxo-2,5-dihydro-1H-pyrrol-1-yl)propanoyl]-L-valyl-L-alanyl}amino)phenyl}ethyl)-L-gluconic Acid

### 2.146.1 (S)-2-(((benzyloxy)carbonyl)amino)-3-(3,4-dihydroxyphenyl)propanoic Acid

To a mixture of (S)-2-amino-3-(3,4-dihydroxyphenyl)propanoic acid (1.00 kg) and NaHCO₃ (1.28 kg) in dioxane (5.00 L) and water (5.00 L) was added benzyl carbonochloridate (1.04 k) dropwise. The reaction mixture was stirred at 25° C. for 12 hours. The reaction mixture was adjusted to pH=3.0˜4.0 by addition of 6 N aqueous HCl and extracted with ethyl acetate (25 L). The organic layer was dried over Na₂SO₄, filtered, and concentrated in vacuo to afford the title compound. ¹H NMR (400 MHz, dimethyl sulfoxide-d₆) δ ppm 8.73 (s, 1H), 7.54-7.26 (m, 8H), 6.64-6.45 (m, 3H), 4.98 (s, 2H), 4.49 (s, 1H), 2.87 (d, J=9.60 Hz, 1H), 2.68-2.62 (m, 1H).

### 2.146.2 (S)-benzyl 2-(((benzyloxy)carbonyl)amino)-3-(3,4-dihydroxyphenyl)propanoate

To a mixture of Example 2.146.1 (800.00 g) and Cs₂CO₃ (1.18 kg) was added bromomethylbenzene (259.67 g) at 20° C. The reaction mixture was stirred for 1 hour, and TLC showed the reaction was complete. The residue was diluted with H₂O (5 L) and extracted with ethyl acetate (three times 5 L). The combined organic layers were washed with brine (5 L), dried over Na₂SO₄ (150 g), filtered, and concentrated under reduce pressure. The residue was purified by column chromatography (SiO₂, petroleum ether/ethyl acetate=100:1 to 1:1) twice to provide the title compound. ¹H NMR (400 MHz, CDCl₃) δ ppm 2.77-3.02 (m, 2H), 4.47 (br. s., 1H), 4.61 (d, J=7.94 Hz, 1H), 5.01-5.17 (m, 4H), 5.35-5.47 (m, 1H), 6.32 (br. s., 1H), 6.38 (d, J=7.94 Hz, 1H), 6.51 (s, 1H), 6.65 (d, J=7.94 Hz, 1H), 7.17-7.42 (m, 9H).

### 2.146.3 (S)-benzyl 2-(((benzyloxy)carbonyl)amino)-3-(3,4-bis(2,5,8,11,14,17,20,23,26,29,32-undecaoxatetratriacontan-34-yloxy)phenyl)propanoate

To a mixture of K₂CO₃ (27.04 g) and KI (5.95 g) in N,N-dimethylformamide (150 mL) was added Example 2.146.2 (8.12 g) and 2,5,8,11,14,17,20,23,26,29,32-undecaoxatetratriacontan-34-yl 4-methylbenzenesulfonate (27.00 g) in dimethylformamide (150 mL). The mixture was stirred at 75° C. for 12 hours under N₂. Two additional vials were set up as described above. All three reaction mixtures were combined for purification. The mixture was poured into NH₄Cl aqueous mixture (9 L), and extracted with ethyl acetate (five times with 900 mL). The combined organic layers were washed with brine (1500 mL), dried over Na₂SO₄ (150 g), filtered, and concentrated under reduce pressure to afford the crude residue. The residue was purified by column chromatography (SiO₂, dichloromethane/methanol=100/1 to 20:1) to provide the title compound. ¹H NMR (400 MHz, CDCl₃) δ ppm 2.95-3.08 (m, 2H), 3.38 (s, 6H), 3.57-3.68 (m, 80H), 3.78 (t, J=4.85 Hz, 2H), 3.83 (t, J=5.29 Hz, 2H), 4.01 (t, J=5.07 Hz, 2H), 4.10 (t, J=5.07 Hz, 2H), 4.58-4.70 (m, 1H), 5.09 (s, 2H), 5.14 (d, J=3.53 Hz, 2H), 6.55 (d, J=8.38 Hz, 1H), 6.62 (d, J=1.76 Hz, 1H), 6.74 (d, J=7.94 Hz, 1H), 7.27-7.49 (m, 10H).

### 2.146.4 (S)-2-amino-3-(3,4-bis(2,5,8,11,14,17,20,23,26,29,32-undecaoxatetratriacontan-34-yloxy)phenyl)propanoic Acid

To a mixture of Example 2.146.3 (16.50 g) in methanol (200 mL) was added Pd/C (9.00 g), and the mixture was stirred at 50° C. under H₂ (50 psi) for 16 hours. An additional reaction was set up as described above. LC/MS showed the reaction was complete, and both reaction mixtures were combined for purification. The mixture was filtered and concentrated. The crude title compound was used in the next step without further purification.

### 2.146.5 (S)-3-(3,4-bis(2,5,8,11,14,17,20,23,26,29,32-undecaoxatetratriacontan-34-yloxy)phenyl)-2-(2,5-dioxo-2,5-dihydro-1H-pyrrol-1-yl)propanoic Acid

To a mixture of Example 2.146.4 (5.94 g) in H₂O (60.00 mL) was added Na₂CO₃ (790.67 mg) and methyl 2,5-dioxopyrrole-1-carboxylate (1.19 g). The mixture was stirred at 25° C. for 3 hours. Four additional reactions were set up as described above. All five reaction mixtures were combined for purification. Aqueous 4M HCl was added to adjust the pH to 2. The combined mixture was purified by preparatory reverse-phase HPLC (trifluoroacetic acid conditions) to provide the title compound. ¹H NMR (400 MHz, CDCl₃) δ ppm 3.35-3.40 (m, 6H), 3.51-3.58 (m, 4H), 3.58-3.75 (m, 78H), 3.81 (q, J=4.70 Hz, 4H), 4.11 (dt, J=10.14, 5.07 Hz, 4H), 4.91 (dd, J=11.47, 5.29 Hz, 1H), 6.53-6.69 (m, 3H), 6.71-6.89 (m, 2H). MS (ESI) m/e6 38.0 (M+H)⁺.

### 2.146.6 (6S)-2,6-anhydro-6-(2-{2-[({[2-({3-[(4-{6-[8-(1,3-benzothiazol-2-ylcarbamoyl)-3,4-dihydroisoquinolin-2(1H)-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](2-sulfoethyl)carbamoyl}oxy)methyl]-5-({N-[(2S)-3-[3,4-bis(2,5,8,11,14,17,20,23,26,29,32-undecaoxatetratriacontan-34-yloxy)phenyl]-2-(2,5-dioxo-2,5-dihydro-1H-pyrrol-1-yl)propanoyl]-L-valyl-L-alanyl}amino)phenyl}ethyl)-L-gluconic Acid

A mixture of Example 2.146.5 (0.020 mL), O-(7-azabenzotriazol-1-yl)-N,N,N′,N′-tetramethyluronium hexafluorophosphate (0.014 g) and N-ethyl-N-isopropylpropan-2-amine (0.020 mL) was stirred in N,N-dimethylformamide (0.4 mL) for 5 minutes. The mixture was added to a mixture of Example 2.123.20 (0.042 g) and N-ethyl-N-isopropylpropan-2-amine (0.020 mL) in N,N-dimethylformamide (0.4 mL) and it was stirred at room temperature for 3 hours. The reaction was diluted with a mixture of water (1.5 mL), N,N-dimethylformamide (0.5 mL) and 2,2,2-trifluoroacetic acid (0.054 mL) and purified by preparatory reverse-phase HPLC on a Gilson 2020 system, using a gradient of 5% to 85% acetonitrile/water. The product-containing fractions were lyophilized to give the title compound. ¹H NMR (501 MHz, dimethyl sulfoxide-d₆) δ 12.86 (s, 4H), 9.92 (s, 2H), 8.26 (d, 1H), 8.10 (s, 1H), 8.02 (dd, 1H), 7.77 (d, 1H), 7.64 (s, 1H), 7.54-7.49 (m, 1H), 7.49-7.39 (m, 2H), 7.39-7.31 (m, 2H), 7.28 (s, 1H), 7.20 (d, 1H), 6.94 (d, 1H), 6.87 (s, 2H), 6.77 (d, 1H), 6.60-6.53 (m, 1H), 5.05-4.91 (m, 5H), 4.80 (dd, 2H), 4.37 (t, 2H), 4.21 (t, 2H), 3.97 (dt, 3H), 3.86 (t, 3H), 3.78 (d, 3H), 3.68 (dt, 4H), 3.65-3.28 (m, 102H), 3.20-3.08 (m, 2H), 2.99 (t, 2H), 2.92 (d, 2H), 2.68 (dd, 2H), 2.07 (d, 4H), 1.54 (s, 2H), 1.37-0.71 (m, 16H). MS (ESI) m/e 2631.2 (M−H)⁻.

### 2.147 Synthesis of N-[(2,5-dioxo-2,5-dihydro-1H-pyrrol-1-yl)acetyl]-N-(2,5,8,11,14,17,20,23,26,29,32-undecaoxatetratriacontan-34-yl)-beta-alanyl-L-valyl-N-{4-[({[2-({3-[(4-{6-[8-(1,3-benzothiazol-2-ylcarbamoyl)-3,4-dihydroisoquinolin-2(1H)-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](2-sulfoethyl)carbamoyl}oxy)methyl]-3-(2,5,8,11,14,17,20,23,26,29,32,35,38,41,44,47,50-heptadecaoxatripentacontan-53-yl)phenyl}-L-alaninamide

### 2.147.1 benzyl 2,5,8,11,14,17,20,23,26,29,32-undecaoxa-35-azaoctatriacontan-38-oate

To a mixture of 2,5,8,11,14,17,20,23,26,29,32-undecaoxatetratriacontan-34-amine (1 g) in N,N-dimethylformamide (4 mL) and water (3 mL) was added benzyl acrylate (0.377 g), dropwise. The reaction mixture was stirred overnight purified by reverse-phase HPLC on a Gilson system (C18 column), eluting with 20-70% acetonitrile in water containing 0.1% trifluoroacetic acid, to give the title compound. MS (ESI) m/e 678.4 (M+H)⁺.

### 2.147.2 2,5,8,11,14,17,20,23,26,29,32-undecaoxa-35-azaoctatriacontan-38-oic Acid

Example 2.147.1 (220 mg) and 10% Pd/C (44 mg, dry) in tetrahydrofuran (10 mL) was shaken in a pressure bottle for 1 hour under 50 psi of hydrogen gas. The reaction was filtered, and the filtrate was concentrated. The residue was dried under high vacuum to provide the title compound. MS (ESI) m/e 588.3 (M+H)⁺.

### 2.147.3 2,5-dioxopyrrolidin-1-yl 35-(2-(2,5-dioxo-2,5-dihydro-1H-pyrrol-1-yl)acetyl)-2,5,8,11,14,17,20,23,26,29,32-undecaoxa-35-azaoctatriacontan-38-oate

A cold (0° C.) mixture of 2,5-dioxopyrrolidin-1-yl 2-(2,5-dioxo-2,5-dihydro-1H-pyrrol-1-yl)acetate (566 mg), 1-hydroxybenzotriazole hydrate (229 mg), 1-hydroxypyrrolidine-2,5-dione (86 mg) and Example 2.147.2 (440 mg) in N,N-dimethylformamide (3 mL) was treated with N,N-diisopropylethylamine (785 μL) for 25 minutes. The reaction was diluted with dimethyl sulfoxide and purified by reverse-phase HPLC on a Gilson system (C18 column), eluting with 5-55% acetonitrile in water containing 0.1% trifluoroacetic acid, to give the title compound. MS (ESI) m/e 822.3 (M+H)⁺.

### 2.147.4 N-[(2,5-dioxo-2,5-dihydro-1H-pyrrol-1-yl)acetyl]-N-(2,5,8,11,14,17,20,23,26,29,32-undecaoxatetratriacontan-34-yl)-beta-alanyl-L-valyl-N-{4-[({[2-({3-[(4-{6-[8-(1,3-benzothiazol-2-ylcarbamoyl)-3,4-dihydroisoquinolin-2(1H)-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](2-sulfoethyl)carbamoyl}oxy)methyl]-3-(2,5,8,11,14,17,20,23,26,29,32,35,38,41,44,47,50-heptadecaoxatripentacontan-53-yl)phenyl}-L-alaninamide

To a cold (0° C.) mixture of Example 2.141.4 (28 mg), Example 2.147.3 (27.1 mg) and 1-hydroxybenzotriazole hydrate (6.6 mg) in N,N-dimethylformamide (0.8 mL) was added N,N-diisopropylethylamine-2 (20.1 μL). The mixture was stirred for 10 minutes and was purified by reverse-phase HPLC on a Gilson system (C18 column), eluting with 30-70% acetonitrile in water containing 0.1% trifluoroacetic acid, to give the title compound. ¹H NMR (400 MHz, dimethyl sulfoxide-d₆) δ 12.81 (s, 1H), 9.84 (s, 1H), 8.21-7.86 (m, 2H), 7.75 (d, 1H), 7.57 (d, 1H), 7.52-7.28 (m, 7H), 7.27-7.15 (m, 2H), 7.04 (d, 2H), 6.91 (d, 1H), 4.94 (d, 4H), 4.36 (dt, 3H), 4.19 (dt, 1H), 3.84 (t, 2H), 3.75 (d, 2H), 3.63 (d, 1H), 3.46 (dd, 104H), 3.36 (s, 2H), 3.19 (s, 5H), 2.97 (t, 2H), 2.57 (t, 5H), 2.42-2.26 (m, 1H), 2.03 (s, 7H), 2.00-1.83 (m, 1H), 1.70 (t, 2H), 1.38-0.96 (m, 13H), 0.96-0.69 (m, 13H). MS (ESI) m/e 1327.7 (M−2H)²⁻.

### 2.148 Synthesis of N-[(2,5-dioxo-2,5-dihydro-1H-pyrrol-1-yl)acetyl]-N-(2,5,8,11,14,17,20,23,26,29,32-undecaoxatetratriacontan-34-yl)-beta-alanyl-L-valyl-N-{4-[({[2-({3-[(4-{6-[8-(1,3-benzothiazol-2-ylcarbamoyl)-3,4-dihydroisoquinolin-2(1H)-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](2-sulfoethyl)carbamoyl}oxy)methyl]phenyl}-N⁵-carbamoyl-L-ornithinamide

The title compound was prepared using the procedure in Example 2.147.4, replacing Example 2.141.4 with Example 2.112.2. ¹H NMR (400 MHz, dimethyl sulfoxide-d₆) δ 12.83 (s, 1H), 9.96 (d, 1H), 8.18-7.85 (m, 3H), 7.75 (d, 1H), 7.64-7.37 (m, 7H), 7.32 (td, 2H), 7.28-7.20 (m, 3H), 7.04 (s, 2H), 6.92 (d, 1H), 5.17-4.79 (m, 4H), 4.59-4.31 (m, 3H), 4.21 (dt, 1H), 3.84 (t, 2H), 3.77 (d, 2H), 3.52 (s, 4H), 3.39 (d, 2H), 3.19 (s, 5H), 2.94 (dt, 4H), 2.60 (t, 3H), 2.43-2.27 (m, 1H), 2.05 (s, 4H), 1.60 (d, 2H), 1.44-0.57 (m, 22H). MS (ESI) m/e 1964.8 (M−H)⁻.

### 2.149 Synthesis of N-[(2,5-dioxo-2,5-dihydro-1H-pyrrol-1-yl)acetyl]-L-valyl-N-{4-[({[2-({3-[(4-{6-[8-(1,3-benzothiazol-2-ylcarbamoyl)-3,4-dihydroisoquinolin-2(1H)-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](2-sulfoethyl)carbamoyl}oxy)methyl]-3-[27-(2,5,8,11,14,17,20,23-octaoxahexacosan-26-yl)-2,5,8,11,14,17,20,23-octaoxa-27-azatriacontan-30-yl]phenyl}-L-alaninamide

### 2.149.1 3-(1-((3-(2-((((2-(27-(2,5,8,11,14,17,20,23-octaoxahexacosan-26-yl)-2,5,8,11,14,17,20,23-octaoxa-27-azatriacontan-30-yl)-4-((S)-2-((S)-2-amino-3-methylbutanamido)propanamido)benzyl)oxy)carbonyl)(2-sulfoethyl)amino)ethoxy)-5,7-dimethyladamantan-1-yl)methyl)-5-methyl-1H-pyrazol-4-yl)-6-(8-(benzo[d]thiazol-2-ylcarbamoyl)-3,4-dihydroisoquinolin-2(1H)-yl)picolinic Acid

The title compound was prepared as described in Example 2.145.5, replacing Example 1.43 with Example 1.2.9. MS (ESI) m/e 1991.4 (M−H)⁻.

### 2.149.2 N-[(2,5-dioxo-2,5-dihydro-1H-pyrrol-1-yl)acetyl]-L-valyl-N-{4-[({[2-({3-[(4-{6-[8-(1,3-benzothiazol-2-ylcarbamoyl)-3,4-dihydroisoquinolin-2(1H)-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](2-sulfoethyl)carbamoyl}oxy)methyl]-3-[27-(2,5,8,11,14,17,20,23-octaoxahexacosan-26-yl)-2,5,8,11,14,17,20,23-octaoxa-27-azatriacontan-30-yl]phenyl}-L-alaninamide

The title compound was prepared as described in Example 2.145, replacing Example 2.145.5 with Example 2.149.1. ¹H NMR (400 MHz, dimethyl sulfoxide-d₆) δ ppm 12.83 (s, 1H), 9.90 (s, 1H), 9.41 (s, 1H), 8.24 (d, 2H), 8.01 (d, 1H), 7.77 (d, 1H), 7.67-7.29 (m, 8H), 7.26 (s, 2H), 7.06 (s, 2H), 6.93 (d, 1H), 5.03 (d, 2H), 4.93 (s, 2H), 4.37 (t, 1H), 4.19 (dd, 1H), 4.11 (s, 2H), 3.86 (t, 2H), 3.79 (s, 2H), 3.70-3.26 (m, 226H), 3.21 (s, 6H), 3.11 (s, 5H), 2.99 (t, 2H), 2.66 (d, 4H), 2.08 (s, 3H), 1.89 (s, 8H), 1.44-0.90 (m, 14H), 0.89-0.68 (m, 11H).

### 2.150 Synthesis of N-{(3S)-3-(2,5-dioxo-2,5-dihydro-1H-pyrrol-1-yl)-3-[1-(2,5,8,11,14,17,20,23,26,29,32,35-dodecaoxaheptatriacontan-37-yl)-1H-1,2,3-triazol-4-yl]propanoyl}-L-valyl-N-{4-[({[2-({3-[(4-{6-[8-(1,3-benzothiazol-2-ylcarbamoyl)-3,4-dihydroisoquinolin-2(1H)-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](2-sulfoethyl)carbamoyl}oxy)methyl]phenyl}-N⁵-carbamoyl-L-ornithinamide

### 2.150.1 3-(2,5-dioxo-2,5-dihydro-1H-pyrrol-1-yl)pent-4-ynoic Acid

To a mixture of 3-aminopent-4-ynoic acid trifluoroacetic acid salt (1.9 g) in tetrahydrofuran (30 mL) was added methyl 2,5-dioxo-2,5-dihydro-1H-pyrrole-1-carboxylate (1.946 g), followed by the rapid addition of N,N-diisopropylethylamine (8.04 mL). The resulting mixture was stirred at 60° C. for 16 hours. The mixture was concentrated to dryness. The residue was purified by reverse-phase HPLC on a Gilson system (C18 column), eluting with 20-80% acetonitrile in water containing 0.1% trifluoroacetic acid, to give the title compound. MS (LC-MS) m/e 194 (M+H). ¹H-NMR (dimethyl sulfoxide-d₆, 400 MHz) δ 2.92-3.07 (m, 2H), 3.38 (d, 1H), 5.07-5.12 (m, 1H), 7.08 (s, 2H), 12.27 (bs, 0.6H).

### 2.150.2 3-(1-(2,5,8,11,14,17,20,23,26,29,32,35-dodecaoxaheptatriacontan-37-yl)-1H-1,2,3-triazol-4-yl)-3-(2,5-dioxo-2,5-dihydro-1H-pyrrol-1-yl)propanoic Acid

To Example 2.150.1 (700 mg) in a mixture of t-butanol/H₂O, (2:1, 15 mL) was added 37-azido-2,5,8,11,14,17,20,23,26,29,32,35-dodecaoxaheptatriacontane (2123 mg). Sodium (R)-2-((S)-1,2-dihydroxyethyl)-4-hydroxy-5-oxo-2,5-dihydrofuran-3-olate (71.8 mg) and copper(II) sulfate (28.9 mg) were sequentially added to the mixture. The resulting mixture was stirred at room temperature for 16 hours and concentrated. The residue was purified by reverse-phase HPLC on a Gilson system (C18 column), eluting with 20-80% acetonitrile in water containing 0.1% trifluoroacetic acid, to give the title compound. ¹H NMR (400 MHz, dimethyl sulfoxide-d₆) δ 3.24 (s, 3H), 3.15-3.28 (m, 2H), 3.41-3.52 (m, 44H), 3.79 (t, 2H), 4.48 (t, 2H), 5.56-5.60 (m, 1H), 7.05 (s, 2H), 8.03 (s, 1H). MS (LC-MS) m/e 779 (M+H)⁺.

### 2.150.3 N-{(3S)-3-(2,5-dioxo-2,5-dihydro-1H-pyrrol-1-yl)-3-[1-(2,5,8,11,14,17,20,23,26,29,32,35-dodecaoxaheptatriacontan-37-yl)-1H-1,2,3-triazol-4-yl]propanoyl}-L-valyl-N-{4-[({[2-({3-[(4-{6-[8-(1,3-benzothiazol-2-ylcarbamoyl)-3,4-dihydroisoquinolin-2(1H)-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](2-sulfoethyl)carbamoyl}oxy)methyl]phenyl}-N⁵-carbamoyl-L-ornithinamide

To a mixture of O-(7-azabenzotriazol-1-yl)-N,N,N′,N′-tetramethyluronium hexafluorophosphate (8.45 mg), and Example 2.150.2 (20 mg) in N,N-dimethylformamide (0.3 mL) at 0° C. was slowly added N,N-diisopropylethylamine (22.19 μL), and the reaction mixture was stirred for 1 minute. A cold (0° C.) mixture of Example 2.112.2 (20 mg) and N,N-diisopropylethylamine (22 μL) in N,N-dimethylformamide (0.4 mL) was added. The resulting mixture was stirred for 10 minutes and was purified by reverse-phase HPLC on a Gilson system (C18 column), eluting with 20-80% acetonitrile in water containing 0.1% trifluoroacetic acid, to give the title compound. (The absolute configuration of the 3-position was arbitrarily assigned.) ¹H NMR (501 MHz, dimethyl sulfoxide-d₆) δ 9.95 (s, 1H), 8.07 (d, 3H), 8.04-7.96 (m, 2H), 7.77 (d, 1H), 7.64-7.53 (m, 3H), 7.50 (s, 1H), 7.48-7.39 (m, 2H), 7.34 (q, 2H), 7.30-7.23 (m, 3H), 6.98 (s, 2H), 6.93 (d, 1H), 5.61 (t, 1H), 4.96 (d, 4H), 4.54-4.27 (m, 3H), 4.14 (t, 1H), 3.86 (t, 2H), 3.77 (q, 4H), 3.43 (d, 71H), 3.21 (s, 6H), 3.00 (d, 5H), 2.61 (s, 2H), 2.07 (d, 3H), 1.92 (s, 1H), 1.60 (d, 2H), 1.47-0.86 (m, 10H), 0.85-0.67 (m, 12H). MS (ESI) m/e 1010.6 (M−2H)²⁻.

### 2.151 Synthesis of N-{(3R)-3-(2,5-dioxo-2,5-dihydro-1H-pyrrol-1-yl)-3-[1-(2,5,8,11,14,17,20,23,26,29,32,35-dodecaoxaheptatriacontan-37-yl)-1H-1,2,3-triazol-4-yl]propanoyl}-L-valyl-N-{4-[({[2-({3-[(4-{6-[8-(1,3-benzothiazol-2-ylcarbamoyl)-3,4-dihydroisoquinolin-2(1H)-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](2-sulfoethyl)carbamoyl}oxy)methyl]phenyl}-N⁵-carbamoyl-L-ornithinamide

Example 2.151 was isolated during the preparation of 2.150.3. (The absolute configuration of the 3-position was arbitrarily assigned.) ¹H NMR (501 MHz, dimethyl sulfoxide-d₆) δ 9.91 (s, 1H), 8.11 (dd, 2H), 8.04-7.99 (m, 1H), 7.96 (s, 1H), 7.77 (d, 1H), 7.58 (t, 3H), 7.54-7.39 (m, 2H), 7.39-7.31 (m, 2H), 7.31-7.24 (m, 3H), 7.00 (s, 2H), 6.94 (d, 1H), 5.61 (dd, 1H), 5.08-4.79 (m, 4H), 4.40 (dt, 3H), 4.16 (s, 1H), 3.86 (t, 2H), 3.82-3.73 (m, 4H), 3.51-3.30 (m, 46H), 3.21 (s, 7H), 3.05-2.87 (m, 3H), 2.62 (t, 2H), 2.07 (d, 3H), 1.95 (s, 2H), 1.69 (s, 1H), 1.51-0.86 (m, 10H), 0.88-0.70 (m, 13H). MS (ESI) m/e 1010.6 (M−2H)²⁻.

### 2.152 Synthesis of 6-[8-(1,3-benzothiazol-2-ylcarbamoyl)-3,4-dihydroisoquinolin-2(1H)-yl]-3-[1-({3-[2-({[(2-{2-[(2S,3R,4R,5S,6S)-6-carboxy-3,4,5-trihydroxytetrahydro-2H-pyran-2-yl]ethyl}-4-{[(2S)-2-{[(2S)-2-{[(2,5-dioxo-2,5-dihydro-1H-pyrrol-1-yl)acetyl]amino}-3-methylbutanoyl]amino}propanoyl]amino}benzyl)oxy]carbonyl}[(3R,4S,5R)-3,4,5,6-tetrahydroxyhexyl]amino)ethoxy]-5,7-dimethyltricyclo[3.3.1.1^{3,7}]dec-1-yl}methyl)-5-methyl-1H-pyrazol-4-yl]pyridine-2-carboxylic Acid

### 2.152.1 3-(1-((3-(2-((((4-((S)-2-((S)-2-amino-3-methylbutanamido)propanamido)-2-(2-((2S,3R,4R,5S,6S)-6-carboxy-3,4,5-trihydroxytetrahydro-2H-pyran-2-yl)ethyl)benzyl)oxy)carbonyl)((3R,4S,5R)-3,4,5,6-tetrahydroxyhexyl)amino)ethoxy)-5,7-dimethyladamantan-1-yl)methyl)-5-methyl-1H-pyrazol-4-yl)-6-(8-(benzo[d]thiazol-2-ylcarbamoyl)-3,4-dihydroisoquinolin-2(1H)-yl)picolinic Acid

The title compound was prepared by substituting Example 1.77.2 for Example 1.25 and Example 2.123.19 for Example 2.97.7 in Example 2.97.8. MS (ESI) m/e 1417 (M+H)⁺, 1415 (M−H)⁺.

### 2.152.2 6-[8-(1,3-benzothiazol-2-ylcarbamoyl)-3,4-dihydroisoquinolin-2(1H)-yl]-3-[1-({3-[2-({[(2-{2-[(2S,3R,4R,5S,6S)-6-carboxy-3,4,5-trihydroxytetrahydro-2H-pyran-2-yl]ethyl}-4-{[(2S)-2-{[(2S)-2-{[(2,5-dioxo-2,5-dihydro-1H-pyrrol-1-yl)acetyl]amino}-3-methylbutanoyl]amino}propanoyl]amino}benzyl)oxy]carbonyl}[(3R,4S,5R)-3,4,5,6-tetrahydroxyhexyl]amino)ethoxy]-5,7-dimethyltricyclo[3.3.1.1^{3,7}]dec-1-yl}methyl)-5-methyl-1H-pyrazol-4-yl]pyridine-2-carboxylic Acid

The title compound was prepared by substituting Example 2.152.1 for Example 2.49.1 in Example 2.54. ¹H NMR (400 MHz, dimethyl sulfoxide-d₆) δ ppm 9.85 (m, 1H), 8.18 (t, 2H), 7.96 (d, 1H), 7.73 (d, 1H), 7.55 (d, 1H), 7.46-7.25 (m, 8H), 7.21 (s, 1H), 7.15 (d, 1H), 7.00 (s, 1H), 6.99 (d, 1H), 6.88 (d, 1H), 4.95 (bs, 2H), 4.88 (s, 2H), 4.32 (m, 1H), 4.15 (t, 1H), 4.05 (s, 2H), 3.82 (t, 2H), 3.72 (m, 4H), 3.58-3.29 (m, 6H), 3.19 (m, 4H), 3.11-3.00 (m, 6H), 2.97 (t, 2H), 2.91 (t, 2H), 2.72 (m, 2H), 2.55 (m, 2H), 2.04 (s, 3H), 2.02-1.85 (m, 3H), 1.54 (m, 4H), 1.44 (s, 1H), 1.33 (bs, 1H), 1.22 (m, 6H), 1.04 (m, 6H), 0.86 (m, 2H), 0.77 (m, 12H). MS (ESI) m/e 1554 (M+H)⁺, 1552 (M−H)⁻.

### 2.153 Synthesis of 6-[8-(1,3-benzothiazol-2-ylcarbamoyl)-3,4-dihydroisoquinolin-2(1H)-yl]-3-[1-({3-[2-({[(2-{2-[(2S,3R,4R,5S,6S)-6-carboxy-3,4,5-trihydroxytetrahydro-2H-pyran-2-yl]ethyl}-4-{[(2S)-2-({(2S)-2-[({(3S,5S)-3-(2,5-dioxo-2,5-dihydro-1H-pyrrol-1-yl)-2-oxo-5-[(2-sulfoethoxy)methyl]pyrrolidin-1-yl}acetyl)amino]-3-methylbutanoyl}amino)propanoyl]amino}benzyl)oxy]carbonyl}[(3R,4S,5R)-3,4,5,6-tetrahydroxyhexyl]amino)ethoxy]-5,7-dimethyltricyclo[3.3.1.1^{3,7}]dec-1-yl}methyl)-5-methyl-1H-pyrazol-4-yl]pyridine-2-carboxylic Acid

Example 2.119.15 (11 mg) was dissolved in N,N-dimethylformamide (0.1 mL). 2-(3H-[1,2,3]triazolo[4,5-b]pyridin-3-yl)-1,1,3,3-tetramethylisouronium hexafluorophosphate(V) (11 mg) and N,N-diisopropylethylamine (7.4 mg) were added. The mixture was stirred at room temperature for five minutes. The mixture was then added to another mixture of Example 2.152.1 (34 mg) and N,N-diisopropylethylamine (16.3 mg) in N,N-dimethylformamide (0.2 mL). The reaction was stirred for 60 minutes at room temperature and quenched with trifluoroacetic acid (36 mg). The mixture was diluted with water (0.75 mL) and dimethyl sulfoxide (0.75 mL) and purified by reverse-phase HPLC using 10-75% acetonitrile in water (w/0.1% TFA) over 30 minutes on a Grace Reveleris equipped with a Luna column: C18(2), 100 A, 150×30 mm. Product fractions were pooled, frozen, and lyophilized to yield the title compound as the trifluoroacetic acid salt. ¹H NMR (400 MHz, dimethyl sulfoxide-d₆) δ ppm 9.85 (m, 1H), 8.18 (d, 1H), 8.05 (d, 1H), 8.04 (d, 1H), 7.79 (d, 1H), 7.53-7.39 (m, 8H), 7.36 (q, 2H), 7.29 (s, 1H), 7.22 (d, 1H), 7.07 (s, 1H), 6.96 (d, 1H), 5.18 (bs, 2H), 4.96 (s, 2H), 4.65 (t, 1H), 4.37 (t, 1H), 4.19 (t, 1H), 4.16 (s, 1H), 4.01 (d, 2H), 3.89 (t, 2H), 3.78 (m, 4H), 3.73 (m, 2H), 3.49-3.44 (m, 4H), 3.40-3.20 (m, 8H), 3.24 (m, 4H), 3.17-3.07 (m, 4H), 3.02 (t, 2H), 2.95 (t, 2H), 2.76 (m, 4H), 2.62 (m, 1H), 2.37 (m, 1H), 2.09 (s, 3H), 1.99 (m, 2H), 1.86 (q, 1H), 1.62 (m, 4H), 1.38 (bs, 2H), 1.28 (m, 6H), 1.18-1.02 (m, 6H), 0.96 (m, 2H), 0.91-0.79 (m, 12H). MS (ESI) m/e 1773 (M−H)⁻.

### 2.154 Synthesis of (6S)-2,6-anhydro-6-(2-{2-[({[2-({3-[(4-{6-[8-(1,3-benzothiazol-2-ylcarbamoyl)-3,4-dihydroisoquinolin-2(1H)-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](2-sulfoethyl)carbamoyl}oxy)methyl]-5-({N-[(2,5-dioxo-2,5-dihydro-1H-pyrrol-1-yl)acetyl]-N-(2,5,8,11,14,17,20,23,26,29,32-undecaoxatetratriacontan-34-yl)-beta-alanyl-L-valyl-L-alanyl}amino)phenyl}ethyl)-L-gluconic Acid

### 2.154.1 3-(1-((3-(2-((((4-((S)-2-((S)-2-amino-3-methylbutanamido)propanamido)-2-(2-((2S,3R,4R,5S,6S)-6-carboxy-3,4,5-trihydroxytetrahydro-2H-pyran-2-yl)ethyl)benzyl)oxy)carbonyl)(2-sulfoethyl)amino)ethoxy)-5,7-dimethyladamantan-1-yl)methyl)-5-methyl-1H-pyrazol-4-yl)-6-(8-yl)picolinic Acid

A mixture of Example 1.2.9 (200 mg), Example 2.123.19 (288 mg), and 1-hydroxybenzotriazole hydrate (50.2 mg) in N,N-dimethylformamide (2 mL) was cooled in an ice-bath, and N,N-diisopropylethylamine (143 μL) was added. The reaction mixture was stirred at room temperature for 2.5 hours and concentrated. Tetrahydrofuran (0.5 mL) and methanol (0.5 mL) were added into the residue. The resulting mixture was cooled in ice-bath and lithium hydroxide hydrate (147 mg) in water (2.5 mL) was slowly added. The mixture was stirred at room temperature for 1.5 hours, and cooled in ice bath. Trifluoroacetic acid (361 μL) was added dropwise until the pH reached 6. The mixture was purified by reverse-phase HPLC on a Gilson system (C18 column), eluting with 35-45% acetonitrile in water containing 0.1% trifluoroacetic acid, to give the title compound. MS (ESI) m/e 1375.5 (M−H)⁻.

### 2.154.2 (6S)-2,6-anhydro-6-(2-{2-[({[2-({3-[(4-{6-[8-(1,3-benzothiazol-2-ylcarbamoyl)-3,4-dihydroisoquinolin-2(1H)-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](2-sulfoethyl)carbamoyl}oxy)methyl]-5-({N-[(2,5-dioxo-2,5-dihydro-1H-pyrrol-1-yl)acetyl]-N-(2,5,8,11,14,17,20,23,26,29,32-undecaoxatetratriacontan-34-yl)-beta-alanyl-L-valyl-L-alanyl}amino)phenyl}ethyl)-L-gluconic Acid

To a mixture of 1-hydroxybenzotriazole hydrate (5.22 mg), Example 2.154.1 (23.5 mg) and Example 2.147.3 (24 mg) in N,N-dimethylformamide (1 mL) at 0° C. was slowly added N,N-diisopropylethylamine (23.84 μL). The reaction mixture was stirred at room temperature for 15 minutes and purified by reverse-phase HPLC on a Gilson system (C18 column), eluting with 35-50% acetonitrile in water containing 0.1% trifluoroacetic acid, to give the title compound. ¹H NMR (501 MHz, dimethyl sulfoxide-d₆) δ 12.83 (s, 1H), 9.88 (s, 1H), 8.23-8.04 (m, 2H), 8.02 (dd, 1H), 7.92 (s, 1H), 7.77 (d, 1H), 7.59 (d, 1H), 7.55-7.30 (m, 7H), 7.27 (s, 1H), 7.20 (d, 1H), 7.07 (d, 2H), 6.93 (d, 1H), 5.07-4.88 (m, 4H), 4.47-4.32 (m, 3H), 4.22 (dt, 1H), 3.97-3.73 (m, 4H), 3.62-3.45 (m, 35H), 3.31 (t, 3H), 3.21 (s, 3H), 3.06 (d, 2H), 2.83-2.54 (m, 5H), 2.47-2.29 (m, 1H), 2.13-1.84 (m, 5H), 1.52 (d, 1H), 1.43-0.69 (m, 26H). MS (ESI) m/e 1043.0 (M−2H)²⁻.

### 2.155 Synthesis of (6S)-2,6-anhydro-6-(2-{2-[({[2-({3-[(4-{6-[8-(1,3-benzothiazol-2-ylcarbamoyl)-3,4-dihydroisoquinolin-2(1H)-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](2-sulfoethyl)carbamoyl}oxy)methyl]-5-[(N-{2-(2,5-dioxo-2,5-dihydro-1H-pyrrol-1-yl)-3-[1-(2,5,8,11,14,17,20,23,26,29,32,35-dodecaoxaheptatriacontan-37-yl)-1H-1,2,3-triazol-4-yl]propanoyl}-L-valyl-L-alanyl)amino]phenyl}ethyl)-L-gluconic Acid

### 2.155.1 3-(1-(2,5,8,11,14,17,20,23,26,29,32,35-dodecaoxaheptatriacontan-37-yl)-1H-1,2,3-triazol-4-yl)-2-(2,5-dioxo-2,5-dihydro-1H-pyrrol-1-yl)propanoic Acid

The title compound was prepared using the procedure in Example 2.150.2, replacing Example 2.150.1 with 2-(2,5-dioxo-2,5-dihydro-1H-pyrrol-1-yl)pent-4-ynoic acid.

### 2.155.2 (6S)-2,6-anhydro-6-(2-{2-[({[2-({3-[(4-{6-[8-(1,3-benzothiazol-2-ylcarbamoyl)-3,4-dihydroisoquinolin-2(1H)-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](2-sulfoethyl)carbamoyl}oxy)methyl]-5-[(N-{2-(2,5-dioxo-2,5-dihydro-1H-pyrrol-1-yl)-3-[1-(2,5,8,11,14,17,20,23,26,29,32,35-dodecaoxaheptatriacontan-37-yl)-1H-1,2,3-triazol-4-yl]propanoyl}-L-valyl-L-alanyl)amino]phenyl}ethyl)-L-gluconic Acid

The title compound was prepared using the procedure in Example 2.150.3, replacing Example 2.150.2 and Example 2.112.2 with Example 2.155.1 and Example 2.154.1, respectively. ¹H NMR (400 MHz, dimethyl sulfoxide-d₆) δ 12.83 (s, 1H), 9.87 (d, 1H), 8.25-8.06 (m, 2H), 8.00 (d, 1H), 7.75 (d, 1H), 7.71 (s, 1H), 7.57 (d, 1H), 7.54-7.28 (m, 6H), 7.25 (s, 1H), 7.18 (d, 1H), 6.98-6.85 (m, 3H), 5.09-4.89 (m, 4H), 4.76 (ddd, 1H), 4.36 (ddd, 3H), 4.17 (q, 1H), 3.84 (t, 2H), 3.76 (d, 2H), 3.72-3.66 (m, 2H), 3.49-3.44 (m, 37H), 3.20 (s, 5H), 3.01-2.82 (m, 3H), 2.13-1.81 (m, 5H), 1.52 (s, 1H), 1.39-0.50 (m, 23H). MS (ESI) m/e 1069.7 (M+2H)²⁺.

### 2.156 Synthesis of (6S)-2,6-anhydro-6-(2-{2-[({[2-({3-[(4-{6-[8-(1,3-benzothiazol-2-ylcarbamoyl)-3,4-dihydroisoquinolin-2(1H)-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](2-sulfoethyl)carbamoyl}oxy)methyl]-5-[(N-{(3S)-3-(2,5-dioxo-2,5-dihydro-1H-pyrrol-1-yl)-3-[1-(2,5,8,11,14,17,20,23,26,29,32,35-dodecaoxaheptatriacontan-37-yl)-1H-1,2,3-triazol-4-yl]propanoyl}-L-valyl-L-alanyl)amino]phenyl}ethyl)-L-gluconic Acid

Example 2.156 was isolated as a pure diastereomer during the preparation of Example 2.155.2. (The assignment of absolute configuration at the 3-position is arbitrary.) ¹H NMR (400 MHz, dimethyl sulfoxide-d₆) δ 12.82 (s, 1H), 9.85 (s, 1H), 8.08 (d, 2H), 8.03-7.95 (m, 2H), 7.75 (d, 1H), 7.57 (d, 1H), 7.51-7.29 (m, 6H), 7.24 (s, 1H), 7.18 (d, 1H), 6.95 (s, 2H), 6.91 (d, 1H), 5.59 (dd, 1H), 5.06-4.86 (m, 4H), 4.43 (dt, 2H), 4.32 (t, 1H), 4.11 (t, 1H), 3.84 (t, 2H), 3.75 (t, 3H), 3.55-3.41 (m, 43H), 3.41-3.36 (m, 2H), 3.19 (s, 5H), 3.10 (t, 1H), 3.03-2.86 (m, 3H), 2.59 (s, 3H), 2.13-1.82 (m, 6H), 1.52 (s, 1H), 1.37-0.65 (m, 26H). MS (ESI) m/e 1067.8 (M−2H)²⁻.

### 2.157 Synthesis of (6S)-2,6-anhydro-6-(2-{2-[({[2-({3-[(4-{6-[8-(1,3-benzothiazol-2-ylcarbamoyl)-3,4-dihydroisoquinolin-2(1H)-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](2-sulfoethyl)carbamoyl}oxy)methyl]-5-[(N-{(3R)-3-(2,5-dioxo-2,5-dihydro-1H-pyrrol-1-yl)-3-[1-(2,5,8,11,14,17,20,23,26,29,32,35-dodecaoxaheptatriacontan-37-yl)-1H-1,2,3-triazol-4-yl]propanoyl}-L-valyl-L-alanyl)amino]phenyl}ethyl)-L-gluconic Acid

Example 2.157 was isolated as a pure diastereomer during the preparation of Example 2.155.2. (The assignment of absolute configuration at the 3-position is arbitrary.) ¹H NMR (400 MHz, dimethyl sulfoxide-d₆) δ 12.81 (s, 1H), 9.81 (s, 1H), 8.10 (d, 2H), 8.00 (d, 1H), 7.94 (s, 1H), 7.75 (d, 1H), 7.57 (d, 1H), 7.51-7.28 (m, 6H), 7.24 (s, 1H), 7.18 (d, 1H), 6.98 (s, 2H), 6.91 (d, 1H), 5.59 (t, 1H), 5.06-4.87 (m, 4H), 4.46-4.26 (m, 2H), 4.12 (d, 1H), 3.84 (t, 2H), 3.75 (d, 3H), 3.46 (d, 27H), 3.40-3.36 (m, 2H), 3.19 (s, 5H), 3.01-2.85 (m, 3H), 2.60 (s, 3H), 1.99 (d, 4H), 1.52 (s, 1H), 1.35-0.65 (m, 23H). MS (ESI) m/e 1067.8 (M−2H)²⁻.

### 2.158 Synthesis of (6S)-2,6-anhydro-6-(2-{2-[({[2-({3-[(4-{6-[8-(1,3-benzothiazol-2-ylcarbamoyl)-3,4-dihydroisoquinolin-2(1H)-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](2-sulfoethyl)carbamoyl}oxy)methyl]-5-[(N-{(3S)-3-(2,5-dioxo-2,5-dihydro-1H-pyrrol-1-yl)-3-[1-(3-sulfopropyl)-1H-1,2,3-triazol-4-yl]propanoyl}-L-valyl-L-alanyl)amino]phenyl}ethyl)-L-gluconic Acid

### 2.158.1 sodium 3-azidopropane-1-sulfonate

To a mixture of sodium azide (3.25 g) in water (25 mL) was added 1, 2-oxathiolane 2,2-dioxide (6.1 g) in acetone (25 mL). The resulting mixture was stirred at room temperature for 24 hours and concentrated to dryness. The solid was suspended in diethyl ether (100 mL) and stirred at reflux for 1 hour. The suspension was cooled to room temperature, and the solid was collected by filtration, washed with acetone and diethyl ether, and dried under vacuum to afford the title compound. MS (LC-MS) m/e 164 (M−H)⁻.

### 2.158.2 isopropyl 3-azidopropane-1-sulfonate

A mixture of Example 2.158.1 (6.8 g) in concentrated HCl (90 mL) was stirred at room temperature for 1 hour. The mixture was concentrated to dryness. The residue was dissolved in dichloromethane (350 mL), and triisopropoxymethane (42.0 mL) was added in one portion to the mixture. The resulting mixture was stirred at 50° C. for 2 hours and concentrated to dryness. The crude residue was purified by silica gel chromatography, eluting with 10/1 petroleum ether/ethyl acetate, to give the title compound. ¹H-NMR (CDCl₃, 400 MHz): 1.42 (s, 3H), 1.44 (s, 3H), 2.08-2.15 (m, 2H), 3.17 (t, 2H), 3.51 (t, 2H), 4.95-5.01 (m, 1H).

### 2.158.3 3-(2,5-dioxo-2,5-dihydro-1H-pyrrol-1-yl)-3-(1-(3-sulfopropyl)-1H-1,2,3-triazol-4-yl)propanoic Acid

To a mixture of Example 2.150.1 (450 mg) in t-butanol/H₂O (2:1, 9 mL) was added Example 2.158.2 (483 mg) followed by copper(II) sulfate (18.59 mg) and sodium (R)-2-((S)-1,2-dihydroxyethyl)-4-hydroxy-5-oxo-2,5-dihydrofuran-3-olate (46.2 mg). The resulting mixture was stirred at room temperature for 16 hours, and the mixture was concentrated to dryness. The residue was purified by reverse-phase HPLC on a Gilson system (C18 column), eluting with 20-80% acetonitrile in water containing 0.1% trifluoroacetic acid, to give the title compound. 1H-NMR (dimethyl sulfoxide-d₆, 400 MHz): 2.06-2.10 (m, 2H), 2.45-2.48 (m, 2H), 3.21-3.23 (m, 2H), 4.40-4.44 (m, 2H), 5.55-5.59 (m, 1H), 7.05 (s, 2H), 8.10 (s, 1H). MS (LCMS) m/e 359 (M+H)⁺.

### 2.158.4 (6S)-2,6-anhydro-6-(2-{2-[({[2-({3-[(4-{6-[8-(1,3-benzothiazol-2-ylcarbamoyl)-3,4-dihydroisoquinolin-2(1H)-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](2-sulfoethyl)carbamoyl}oxy)methyl]-5-[(N-{(3S)-3-(2,5-dioxo-2,5-dihydro-1H-pyrrol-1-yl)-3-[1-(3-sulfopropyl)-1H-1,2,3-triazol-4-yl]propanoyl}-L-valyl-L-alanyl)amino]phenyl}ethyl)-L-gluconic Acid

The title compound was prepared using the procedure in Example 2.150.3, replacing Example 2.150.2 and Example 2.112.2 with Example 2.158.3 and Example 2.154.1, respectively. The compound was isolated as a pure diastereomer. (The absolute configuration of the 3-position was arbitrarily assigned.) ¹H NMR (400 MHz, dimethyl sulfoxide-d₆) δ 10.14-9.66 (m, 1H), 8.07 (d, 2H), 8.04-7.96 (m, 2H), 7.75 (d, 1H), 7.57 (d, 1H), 7.52-7.29 (m, 7H), 7.26 (s, 1H), 7.18 (d, 1H), 6.92 (d, 3H), 5.58 (t, 1H), 5.09-4.84 (m, 4H), 4.35 (dt, 3H), 4.15-4.02 (m, 1H), 3.89-3.65 (m, 4H), 3.28 (d, 1H), 3.21 (dd, 2H), 3.14-3.02 (m, 2H), 3.01-2.86 (m, 4H), 2.62 (d, 3H), 2.37 (t, 2H), 2.29 (s, OH), 2.02 (dt, 5H), 1.52 (s, 1H), 1.40-0.59 (m, 24H). MS (ESI) m/e 1715.3 (M−H)⁻.

### 2.159 Synthesis of (6S)-2,6-anhydro-6-(2-{2-[({[2-({3-[(4-{6-[8-(1,3-benzothiazol-2-ylcarbamoyl)-3,4-dihydroisoquinolin-2(1H)-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](2-sulfoethyl)carbamoyl}oxy)methyl]-5-[(N-{(3R)-3-(2,5-dioxo-2,5-dihydro-1H-pyrrol-1-yl)-3-[1-(3-sulfopropyl)-1H-1,2,3-triazol-4-yl]propanoyl}-L-valyl-L-alanyl)amino]phenyl}ethyl)-L-gluconic Acid

Example 2.159 was isolated as a pure diastereomer during the preparation of Example 2.158. (The absolute configuration of the 3-position was arbitrarily assigned.) ¹H NMR (400 MHz, dimethyl sulfoxide-d₆) δ 9.97 (d, 1H), 8.21 (d, 1H), 8.13 (d, 1H), 8.04-7.96 (m, 2H), 7.75 (d, 1H), 7.57 (d, 1H), 7.55-7.37 (m, 4H), 7.36-7.25 (m, 3H), 7.17 (d, 1H), 6.98 (s, 2H), 6.93 (d, 1H), 5.58 (t, 1H), 4.94 (d, 4H), 4.50-4.26 (m, 3H), 4.10 (s, 1H), 3.98-3.73 (m, 3H), 3.51 (d, 1H), 3.42 (s, 3H), 3.34-3.01 (m, 6H), 3.01-2.83 (m, 4H), 2.63 (d, 4H), 2.42 (d, 1H), 2.18-1.80 (m, 8H), 1.53 (s, 1H), 1.39-0.68 (m, 27H). MS (ESI) m/e 1715.4 (M−H)⁻.

### 2.160 Synthesis of (6S)-2,6-anhydro-6-(2-{2-[({[2-({3-[(4-{6-[8-(1,3-benzothiazol-2-ylcarbamoyl)-3,4-dihydroisoquinolin-2(1H)-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](2-sulfoethyl)carbamoyl}oxy)methyl]-5-({N-[(2,5-dioxo-2,5-dihydro-1H-pyrrol-1-yl)acetyl]-N-[2-(2-sulfoethoxy)ethyl]-beta-alanyl-L-valyl-L-alanyl}amino)phenyl}ethyl)-L-gluconic Acid

### 2.160.1 4-((tert-butyldiphenylsilyl)oxy)-2,2-dimethylbutyl 2-(2-((tert-butoxycarbonyl)amino)ethoxy)ethanesulfonate

To a mixture of tert-butyl (2-hydroxyethyl)carbamate (433 mg) in dimethyl sulfoxide (0.9 mL) at 20° C. were added 4-((tert-butyldiphenylsilyl)oxy)-2,2-dimethylbutyl ethenesulfonate (500 mg) and K₂CO₃ (210 mg). The mixture was warmed to 60° C. and stirred for 16 hours in a capped bottle. The mixture was diluted with ethyl acetate, washed with water and brine. The organic layer was dried over anhydrous sodium sulfate, filtered, and concentrated. The residue was purified by silica gel flash chromatography, eluting with petrol ether/ethyl acetate (10:1˜2:1), to give the title compound. MS (LC-MS) m/e 630.3 (M+Na)⁺.

### 2.160.2 4-((tert-butyldiphenylsilyl)oxy)-2,2-dimethylbutyl 2-(2-aminoethoxy)ethanesulfonate

To a mixture of Example 2.160.1 (1.5 g) in anhydrous dichloromethane (100 mL) at 20° C. was added zinc (II) bromide (0.445 g). The mixture was stirred at room temperature for 16 hours. Additional zinc (II) bromide (278 mg) was added to above mixture, and the reaction was stirred for additional 16 hours. The reaction was quenched with 1 M aqueous Na₂CO₃ mixture (5 mL), and the aqueous layer was extracted with ethyl acetate three times. The combined organic layers were dried over sodium sulfate, filtered, and concentrated. The residue was purified by silica gel column chromatography, eluting with dichloromethane/methanol (10:1), to give the title compound. MS (LC-MS) m/e 508.2 (M+H)⁺.

### 2.160.3 tert-butyl 3-((2-(2-((4-((tert-butyldiphenylsilyl)oxy)-2,2-dimethylbutoxy)sulfonyl)ethoxy)ethyl)amino)propanoate

To a mixture of Example 2.160.2 (0.365 g) in N, N-dimethylformamide (5.5 mL) and water (0.55 mL) were added tert-butyl acrylate (0.105 mL) and triethylamine (10.02 μL). The mixture was stirred at 60° C. for 30 hours. The mixture was concentrated. The residue was mixed with 1 M aqueous Na₂CO₃ mixture (5 mL). The aqueous layer was extracted with ethyl acetate three times. The combined organic layers were dried over sodium sulfate, filtered and concentrated. The residue was purified by silica gel column chromatography, eluting with dichloromethane/ethyl acetate (3:1) and dichloromethane/methanol (10:1), to give the title compound. MS (LC-MS) m/e 636.3 (M+H)⁺.

### 2.160.4 tert-butyl 3-(N-(2-(2-((4-((tert-butyldiphenylsilyl)oxy)-2,2-dimethylbutoxy)sulfonyl)ethoxy)ethyl)-2-(2,5-dioxo-2,5-dihydro-1H-pyrrol-1-yl)acetamido)propanoate

To a mixture of Example 2.160.3 (557.5 mg), 2-(2,5-dioxo-2,5-dihydro-1H-pyrrol-1-yl) acetic acid (272 mg) and O-(7-azabenzotriazol-1-yl)-N,N,N′,N′-tetramethyluronium hexafluorophosphate (667 mg) in N, N-dimethylformamide (1.75 mL) at 0° C. was added N,N-diisopropylethylamine (0.459 mL). The resulting mixture was stirred at 0° C. for 1 hour. The reaction mixture was mixed with saturated aqueous NH₄Cl mixture, extracted with ethyl acetate and washed with brine. The organic layer was dried over sodium sulfate, filtered and concentrated. The residue was purified by silica gel column chromatography, eluting with petroleum ether/ethyl acetate (2/1), to provide the title compound. MS (LC-MS) m/e 795.3 (M+Na)⁺.

### 2.160.5 3-(2-(2,5-dioxo-2,5-dihydro-1H-pyrrol-1-yl)-N-(2-(2-sulfoethoxy)ethyl)acetamido)propanoic Acid

To a mixture of Example 2.160.4 (230 mg) in dichloromethane (4 mL) was added trifluoroacetic acid (3 mL). The mixture was stirred at 20° C. for 16 hours and was concentrated. The residue was purified by reverse-phase HPLC on a Gilson system (C18 column), eluting with 20-80% acetonitrile in water containing 0.1% trifluoroacetic acid, to give the title compound. MS (LC-MS) m/e 379.0 (M+Na)⁺.

### 2.160.6 2-(2-(2-(2,5-dioxo-2,5-dihydro-1H-pyrrol-1-yl)-N-(3-((2,5-dioxopyrrolidin-1-yl)oxy)-3-oxopropyl)acetamido)ethoxy) ethane-1-sulfonic Acid

A mixture of 1-hydroxypyrrolidine-2,5-dione (16.43 mg), Example 2.160.5 (30 mg), 1-ethyl-3-[3-(dimethylamino)propyl]-carbodiimide hydrochloride (45.6 mg) in N,N-dimethylformamide were stirred overnight. The reaction mixture was purified by reverse-phase HPLC on a Gilson system (C18 column), eluting with 2-30% acetonitrile in water containing 0.1% trifluoroacetic acid, to give the title compound. MS (ESI) m/e 475.9 (M+H)⁺.

### 2.160.7 (6S)-2,6-anhydro-6-(2-{2-[({[2-({3-[(4-{6-[8-(1,3-benzothiazol-2-ylcarbamoyl)-3,4-dihydroisoquinolin-2(1H)-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl](2-sulfoethyl)carbamoyl}oxy)methyl]-5-({N-[(2,5-dioxo-2,5-dihydro-1H-pyrrol-1-yl)acetyl]-N-[2-(2-sulfoethoxy)ethyl]-beta-alanyl-L-valyl-L-alanyl}amino)phenyl}ethyl)-L-gluconic Acid

To a mixture of 1-hydroxybenzotriazole hydrate (4.45 mg), Example 2.160.6 (8.97 mg) and Example 2.154.1 (20 mg) in N,N-dimethylformamide (0.8 mL) at 0° C. was added N,N-diisopropylethylamine (20 μL dropwise). The reaction mixture was stirred at room temperature for 1 hour and purified by reverse-phase HPLC on a Gilson system (C18 column), eluting with 30-55% acetonitrile in water containing 0.1% trifluoroacetic acid, to give the title compound. ¹H NMR (500 MHz, dimethyl sulfoxide-d₆) δ 12.87 (s, 1H), 9.88 (d, 1H), 8.28-8.10 (m, 1H), 8.03 (d, 1H), 7.95 (d, 1H), 7.78 (d, 1H), 7.60 (d, 1H), 7.56-7.31 (m, 7H), 7.28 (s, 1H), 7.21 (d, 1H), 7.06 (d, 2H), 6.95 (d, 1H), 5.06-4.90 (m, 4H), 4.38 (q, 3H), 4.28-4.11 (m, 1H), 3.87 (t, 2H), 3.79 (d, 2H), 3.71-3.49 (m, 5H), 3.21 (d, 2H), 3.12 (q, 2H), 2.97 (dt, 3H), 2.84-2.57 (m, 6H), 2.38 (dd, 1H), 2.13-1.86 (m, 5H), 1.55 (s, 1H), 1.39-0.64 (m, 25H). MS (ESI) m/e 867.6 (M−2H)²⁻.

### 2.161 Synthesis of 6-{8-[(1,3-benzothiazol-2-yl) carbamoyl]-3,4-dihydroisoquinolin-2(1H)-yl}-3-[1-({3-[2-({[(2-{2-[(2S,3R,4R,5S,6S)-6-carboxy-3,4,5-trihydroxyoxan-2-yl]ethyl}-4-{[(2S)-2-{[(2S)-2-{[(2S)-2-(2,5-dioxo-2,5-dihydro-1H-pyrrol-1-yl)-3-{4-[(2,5,8,11,14,17,20,23,26,29,32-undecaoxatetratriacontan-34-yl)oxy]phenyl}propanoyl]amino}-3-methylbutanoyl]amino}propanoyl]amino}phenyl)methoxy]carbonyl}[(3R,4S,5R)-3,4,5,6-tetrahydroxyhexyl]amino)ethoxy]-5,7-dimethyltricyclo[3.3.1.1^{3,7}]decan-1-yl}methyl)-5-methyl-1H-pyrazol-4-yl]pyridine-2-carboxylic Acid

The title compound was prepared by substituting Example 2.120.5 for Example 2.119.15 in Example 2.153. ¹H NMR (400 MHz, dimethyl sulfoxide-d₆) δ ppm 12.84 (bs, 2H), 9.92 (m, 1H), 8.26 (d, 1H), 8.13 (d, 1H), 8.03 (d, 1H), 7.79 (d, 1H), 7.61 (d, 1H), 7.52-7.41 (m, 4H), 7.36 (m, 3H), 7.27 (s, 1H), 7.21 (d, 1H), 7.02 (d, 2H), 6.95 (d, 1H), 6.89 (s, 2H), 6.78 (d, 2H), 5.02 (bs, 4H), 4.96 (s, 2H), 4.59 (dd, 1H), 4.38 (m, 2H), 4.21 (t, 1H), 3.99 (t, 2H), 3.88 (t, 2H), 3.79 (m, 2H), 3.69 (t, 2H), 3.64 (m, 1H), 3.57 (m, 4H), 3.53 (m, 4H), 3.50 (s, 40H), 3.42 (m, 2H), 3.38 (m, 1H), 3.30 (m, 2H), 3.23 (s, 6H), 3.20-3.08 (m, 6H), 3.01 (t, 2H), 2.94 (t, 1H), 2.76 (m, 1H), 2.61 (m, 1H), 2.08 (s, 3H), 2.06-1.92 (m, 2H), 1.67-1.52 (m, 3H), 1.38 (m, 1H), 1.32-1.22 (m, 6H), 1.18-1.01 (m, 6H), 0.92 (m, 2H), 0.84 (m, 6H), 0.78 (m, 6H). MS (ESI) m/e 1078 (M−2H).

### 2.162 Synthesis of 4-{[({2-[(3-{[4-(6-{8-[(1,3-benzothiazol-2-yl) carbamoyl]-3,4-dihydroisoquinolin-2(1H)-yl}-2-carboxypyridin-3-yl)-5-methyl-1H-pyrazol-1-yl]methyl}-5,7-dimethyltricyclo[3.3.1.1^{3,7}]decan-1-yl)oxy]ethyl}[(3S)-3,4-dihydroxybutyl]carbamoyl)oxy]methyl}-3-(2-{2-[2-(2,5-dioxo-2,5-dihydro-1H-pyrrol-1-yl)acetamido]ethoxy}ethoxy)phenyl Beta-D-Glucopyranosiduronic Acid

### 2.162.1 3-(1-((3-(2-((((2-(2-(2-aminoethoxy)ethoxy)-4-(((2S,3R,4S,5S,6S)-6-carboxy-3,4,5-trihydroxytetrahydro-2H-pyran-2-yl)oxy)benzyl)oxy)carbonyl)((S)-3,4-dihydroxybutyl)amino)ethoxy)-5,7-dimethyladamantan-1-yl)methyl)-5-methyl-1H-pyrazol-4-yl)-6-(8-yl)picolinic Acid

Example 2.162.1 was prepared by substituting Example 2.62.6 for (9H-fluoren-9-yl)methyl ((S)-3-methyl-1-(((S)-1-((4-((((4-nitrophenoxy)carbonyl)oxy)methyl)phenyl)amino)-1-oxo-5-ureidopentan-2-yl)amino)-1-oxobutan-2-yl)carbamate and substituting Example 1.85 for Example 1.2.9 in Example 2.49.1. MS (ESI) m/e 1261.4 (M−H)⁻.

### 2.162.2 4-{[({2-[(3-{[4-(6-{8-[(1,3-benzothiazol-2-yl) carbamoyl]-3,4-dihydroisoquinolin-2(1H)-yl}-2-carboxypyridin-3-yl)-5-methyl-1H-pyrazol-1-yl]methyl}-5,7-dimethyltricyclo[3.3.1.1^{3,7}]decan-1-yl)oxy]ethyl}[(3S)-3,4-dihydroxybutyl]carbamoyl)oxy]methyl}-3-(2-{2-[2-(2,5-dioxo-2,5-dihydro-1H-pyrrol-1-yl)acetamido]ethoxy}ethoxy)phenyl Beta-D-Glucopyranosiduronic Acid

Example 2.162.2 was prepared by substituting Example 2.162.1 for Example 2.49.1 in Example 2.54. ¹H NMR (400 MHz, dimethyl sulfoxide-d₆) δ 8.18 (t, 1H), 8.00 (dd, 1H), 7.76 (d, 1H), 7.58 (dd, 1H), 7.50-7.29 (m, 6H), 7.26 (s, 1H), 7.17 (d, 1H), 7.03 (s, 2H), 6.92 (d, 1H), 6.64 (d, 1H), 6.57 (dd, 1H), 4.94 (d, 4H), 4.08 (hept, 2H), 4.00 (s, 2H), 3.92-3.68 (m, 8H), 3.51-3.13 (m, 12H), 2.98 (t, 2H), 2.06 (s, 3H), 1.65 (s, 1H), 1.43-0.66 (m, 18H). MS (ESI) m/e 1398.5 (M−H)⁻.

### 2.163 Synthesis of 2,6-anhydro-8-[2-({[{2-[(3-{[4-(6-{8-[(1,3-benzothiazol-2-yl) carbamoyl]-3,4-dihydroisoquinolin-2(1H)-yl}-2-carboxypyridin-3-yl)-5-methyl-1H-pyrazol-1-yl]methyl}-5,7-dimethyltricyclo[3.3.1.1^{3,7}]decan-1-yl)oxy]ethyl}(2-sulfoethyl)carbamoyl]oxy}methyl)-5-{[(79S,82S)-74-[(2,5-dioxo-2,5-dihydro-1H-pyrrol-1-yl)acetyl]-82-methyl-77,80,83-trioxo-79-(propan-2-yl)-2,5,8,11,14,17,20,23,26,29,32,35,38,41,44,47,50,53,56,59,62,65,68,71-tetracosaoxa-74,78,81-triazatrioctacontan-83-yl]amino}phenyl]-7,8-dideoxy-L-glycero-L-gulo-octonic Acid

### 2.163.1 benzyl 2,5,8,11,14,17,20,23,26,29,32,35,38,41,44,47,50,53,56,59,62,65,68,71-tetracosaoxa-74-azaheptaheptacontan-77-oate

The title compound was prepared using the procedure in Example 2.147.1, replacing 2,5,8,11,14,17,20,23,26,29,32-undecaoxatetratriacontan-34-amine with 2,5,8,11,14,17,20,23,26,29,32,35,38,41,44,47,50,53,56,59,62,65,68,71-tetracosaoxatriheptacontan-73-amine. MS (ESI) m/e 625.9 (M+2H)²⁺.

### 2.163.2 2,5,8,11,14,17,20,23,26,29,32,35,38,41,44,47,50,53,56,59,62,65,68,71-tetracosaoxa-74-azaheptaheptacontan-77-oic Acid

The title compound was prepared using the procedure in Example 2.147.2, replacing Example 2.147.1 with Example 2.163.1. MS (ESI) m/e 1160.7 (M+H)⁺.

### 2.163.3 2,5-dioxopyrrolidin-1-yl 74-(2-(2,5-dioxo-2,5-dihydro-1H-pyrrol-1-yl)acetyl)-2,5,8,11,14,17,20,23,26,29,32,35,38,41,44,47,50,53,56,59,62,65,68,71-tetracosaoxa-74-azaheptaheptacontan-77-oate

The title compound was prepared using the procedure in Example 2.147.3, replacing Example 2.147.2 with Example 2.163.2. MS (ESI) m/e 698.1 (M+2H)²⁺.

### 2.163.4 2,6-anhydro-8-[2-({[{2-[(3-{[4-(6-{8-[(1,3-benzothiazol-2-yl) carbamoyl]-3,4-dihydroisoquinolin-2(1H)-yl}-2-carboxypyridin-3-yl)-5-methyl-1H-pyrazol-1-yl]methyl}-5,7-dimethyltricyclo[3.3.1.1^{3,7}]decan-1-yl)oxy]ethyl}(2-sulfoethyl)carbamoyl]oxy}methyl)-5-{[(79S,82S)-74-[(2,5-dioxo-2,5-dihydro-1H-pyrrol-1-yl)acetyl]-82-methyl-77,80,83-trioxo-79-(propan-2-yl)-2,5,8,11,14,17,20,23,26,29,32,35,38,41,44,47,50,53,56,59,62,65,68,71-tetracosaoxa-74,78,81-triazatrioctacontan-83-yl]amino}phenyl]-7,8-dideoxy-L-glycero-L-gulo-octonic Acid

The title compound was prepared using the procedure in Example 2.147.4, replacing Example 2.147.3 and Example 2.141.4 with Example 2.163.3 and Example 2.154.1, respectively. ¹H NMR (400 MHz, dimethyl sulfoxide-d₆) δ 9.86 (s, 1H), 8.23-7.87 (m, 3H), 7.76 (d, 1H), 7.58 (dd, 1H), 7.53-7.25 (m, 7H), 7.19 (d, 1H), 7.05 (d, 2H), 6.92 (d, 1H), 5.07-4.85 (m, 4H), 4.49-4.30 (m, 3H), 4.20 (dt, 1H), 3.52 (d, 8H), 3.46-3.26 (m, 7H), 3.20 (s, 4H), 3.15-2.82 (m, 4H), 2.61 (s, 3H), 2.38 (dq, 1H), 2.11-1.82 (m, 5H), 1.53 (s, 1H), 1.39-0.66 (m, 24H). MS (ESI) m/e 1326.9 (M−2H)²⁻.

### 2.164 Synthesis of 6-{8-[(1,3-benzothiazol-2-yl) carbamoyl]-3,4-dihydroisoquinolin-2(1H)-yl}-3-{1-[(3-{2-[{[(4-{[(2S,5S)-2-[3-(carbamoylamino)propyl]-10-[(2,5-dioxo-2,5-dihydro-1H-pyrrol-1-yl)acetyl]-4,7-dioxo-5-(propan-2-yl)-15-sulfo-13-oxa-3,6,10-triazapentadecanan-1-oyl]amino}phenyl)methoxy]carbonyl}(2-sulfoethyl)amino]ethoxy}-5,7-dimethyltricyclo[3.3.1.1^{3,7}]decan-1-yl)methyl]-5-methyl-1H-pyrazol-4-yl}pyridine-2-carboxylic Acid

A mixture of 1-hydroxypyrrolidine-2,5-dione (2.74 mg), 1-ethyl-3-[3-(dimethylamino)propyl]-carbodiimide hydrochloride (4.26 mg) and Example 2.160.5 (9.01 mg) in N,N-dimethylformamide (0.3 mL) were stirred at room temperature overnight. The mixture was cooled in ice bath. 1-Hydroxybenzotriazole hydrate (3.65 mg) and a mixture of Example 2.112.2 (20 mg) and N,N-diisopropylethylamine (22.19 μL) were added. The resulting mixture was stirred at 0° C. for 10 minutes and purified by reverse phase HPLC, eluting with 30%-55% acetonitrile in 0.1% trifluoroacetic acid water, to provide the title compound. ¹H NMR (400 MHz, dimethyl sulfoxide-d₆) δ 9.95 (d, 1H), 8.18-7.89 (m, 3H), 7.76 (d, 1H), 7.57 (d, 3H), 7.52-7.21 (m, 8H), 7.04 (d, 2H), 6.92 (d, 1H), 4.94 (d, 4H), 4.37 (d, 2H), 4.19 (d, 1H), 3.85 (t, 2H), 3.77 (d, 2H), 3.22 (d, 2H), 2.96 (dt, 4H), 2.73 (dt, 2H), 2.66-2.55 (m, 2H), 2.36 (s, 1H), 2.06 (s, 3H), 1.91 (s, 1H), 1.61 (d, 3H), 1.47-0.86 (m, 11H), 0.80 (ddd, 12H). MS (ESI) m/e 1617.5 (M−H)⁻.

### 2.165 this Paragraph was Intentionally Left Blank

### 2.166 Synthesis of 6-(8-(benzo[d]thiazol-2-ylcarbamoyl)-3,4-dihydroisoquinolin-2(1H)-yl)-3-(1-((3-(2-((((2-(2-((2S,3R,4R,5S,6S)-6-carboxy-3,4,5-trihydroxytetrahydro-2H-pyran-2-yl)ethyl)-4-((S)-2-((S)-2-(2-((3S,5S)-3-(2,5-dioxo-2,5-dihydro-1H-pyrrol-1-yl)-2-oxo-5-((2-sulfoethoxy)methyl)pyrrolidin-1-yl)acetamido)-3-methylbutanamido)propanamido)benzyl)oxy)carbonyl)((S)-3,4-dihydroxybutyl)amino)ethoxy)-5,7-dimethyladamantan-1-yl)methyl)-5-methyl-1H-pyrazol-4-yl)picolinic Acid

The title compound was prepared by substituting Example 2.167.1 for Example 2.119.16 in Example 2.119.17. ¹H NMR (500 MHz, dimethyl sulfoxide-d₆) δ ppm 9.86 (br d, 1H), 8.17 (br d, 1H), 8.04 (m, 2H), 7.78 (d, 1H), 7.61 (d, 1H), 7.51 (br d, 1H), 7.49-7.39 (m, 4H), 7.36 (m, 2H), 7.29 (s, 1H), 7.21 (d, 1H), 7.07 (s, 2H), 6.95 (d, 1H), 5.00 (s, 2H), 4.96 (s, 2H), 4.64 (t, 1H), 4.36 (m, 1H), 4.19 (m, 1H), 4.16 (d, 1H), 4.01 (d, 1H), 3.88 (br t, 2H), 3.82 (br m, 3H), 3.75 (br m, 1H), 3.64 (t, 2H), 3.54 (d, 2H), 3.47 (m, 4H), 3.43 (br m, 4H), 3.23 (br m, 5H), 3.13 (t, 1H), 3.10 (br m, 1H), 3.01 (br m, 2H), 2.93 (t, 1H), 2.83-2.68 (m, 3H), 2.37 (m, 1H), 2.08 (s, 3H), 1.99 (br m, 2H), 1.85 (m, 1H), 1.55 (br m, 1H), 1.37 (br m, 1H), 1.28 (br m, 6H), 1.10 (br m, 7H), 0.93 (br m, 1H), 0.88, 0.85, 0.83 0.79 (d, d, s, s, total 12H). MS (ESI) m/e 1713.6 (M−H)⁻.

### 2.167 Synthesis of 2,6-anhydro-8-(2-{[({2-[(3-{[4-(6-{8-[(1,3-benzothiazol-2-yl) carbamoyl]-3,4-dihydroisoquinolin-2(1H)-yl}-2-carboxypyridin-3-yl)-5-methyl-1H-pyrazol-1-yl]methyl}-5,7-dimethyltricyclo[3.3.1.1^{3,7}]decan-1-yl)oxy]ethyl}[(3S)-3,4-dihydroxybutyl]carbamoyl)oxy]methyl}-5-{[(2S)-2-({(2S)-2-[2-(2,5-dioxo-2,5-dihydro-1H-pyrrol-1-yl)acetamido]-3-methylbutanoyl}amino)propanoyl]amino}phenyl)-7,8-dideoxy-L-glycero-L-gulo-octonic Acid

### 2.167.1 3-(1-((3-(2-((((4-((S)-2-((S)-2-amino-3-methylbutanamido)propanamido)-2-(2-((2S,3R,4R,5S,6S)-6-carboxy-3,4,5-trihydroxytetrahydro-2H-pyran-2-yl)ethyl)benzyl)oxy)carbonyl)((S)-3,4-dihydroxybutyl)amino)ethoxy)-5,7-dimethyladamantan-1-yl)methyl)-5-methyl-1H-pyrazol-4-yl)-6-(8-(benzo[d]thiazol-2-ylcarbamoyl)-3,4-dihydroisoquinolin-2(1H)-yl)picolinic Acid

Example 2.167.1 was prepared by substituting Example 2.123.19 for (9H-fluoren-9-yl)methyl ((S)-3-methyl-1-(((S)-1-((4-((((4-nitrophenoxy)carbonyl)oxy)methyl)phenyl)amino)-1-oxo-5-ureidopentan-2-yl)amino)-1-oxobutan-2-yl)carbamate and substituting Example 1.85 for Example 1.2.9 in Example 2.49.1. MS (ESI) m/e 1355.5 (M−H)⁻.

### 2.167.2 2,6-anhydro-8-(2-{[({2-[(3-{[4-(6-{8-[(1,3-benzothiazol-2-yl) carbamoyl]-3,4-dihydroisoquinolin-2(1H)-yl}-2-carboxypyridin-3-yl)-5-methyl-1H-pyrazol-1-yl]methyl}-5,7-dimethyltricyclo[3.3.1.1^{3,7}]decan-1-yl)oxy]ethyl}[(3S)-3,4-dihydroxybutyl]carbamoyl)oxy]methyl}-5-{[(2S)-2-({(2S)-2-[2-(2,5-dioxo-2,5-dihydro-1H-pyrrol-1-yl)acetamido]-3-methylbutanoyl}amino)propanoyl]amino}phenyl)-7,8-dideoxy-L-glycero-L-gulo-octonic Acid

Example 2.167.2 was prepared by substituting Example 2.167.1 for Example 2.49.1 in Example 2.54. ¹H NMR (501 MHz, dimethyl sulfoxide-d₆) δ 9.90 (d, 1H), 8.25 (m, 2H), 8.01 (d, 1H), 7.77 (d, 1H), 7.59 (d, 1H), 7.51-7.40 (m, 4H), 7.40-7.31 (m, 3H), 7.26 (s, 1H), 7.20 (d, 1H), 7.05 (s, 2H), 6.93 (d, 1H), 4.96 (d, 4H), 4.36 (t, 1H), 4.22-4.06 (m, 3H), 3.85 (t, 2H), 3.26-3.17 (m, 4H), 3.14-2.88 (m, 5H), 2.78-2.55 (m, 2H), 2.10-1.88 (m, 5H), 1.69-1.49 (m, 2H), 1.39-0.73 (m, 28H). MS (ESI) m/e 1492.5 (M−H)⁻.

### 2.168 Synthesis of 2-{[({2-[(3-{[4-(6-{8-[(1,3-benzothiazol-2-yl) carbamoyl]-3,4-dihydroisoquinolin-2(1H)-yl}-2-carboxypyridin-3-yl)-5-methyl-1H-pyrazol-1-yl]methyl}-5,7-dimethyltricyclo[3.3.1.1^{3,7}]decan-1-yl)oxy]ethyl}[(3S)-3,4-dihydroxybutyl]carbamoyl)oxy]methyl}-5-{4-[2-(2,5-dioxo-2,5-dihydro-1H-pyrrol-1-yl)acetamido]butyl}phenyl Beta-D-Glucopyranosiduronic Acid

### 2.168.1 3-(1-((3-(2-((((4-(4-aminobutyl)-2-(((2S,3R,4S,5S,6S)-6-carboxy-3,4,5-trihydroxytetrahydro-2H-pyran-2-yl)oxy)benzyl)oxy)carbonyl)((S)-3,4-dihydroxybutyl)amino)ethoxy)-5,7-dimethyladamantan-1-yl)methyl)-5-methyl-1H-pyrazol-4-yl)-6-(8-(benzo[d]thiazol-2-ylcarbamoyl)-3,4-dihydroisoquinolin-2(1H)-yl)picolinic Acid

Example 2.168.1 was prepared by substituting Example 2.124.5 for (9H-fluoren-9-yl)methyl ((S)-3-methyl-1-(((S)-1-((4-((((4-nitrophenoxy)carbonyl)oxy)methyl)phenyl)amino)-1-oxo-5-ureidopentan-2-yl)amino)-1-oxobutan-2-yl)carbamate and substituting Example 1.85 for Example 1.2.9 in Example 2.49.1. MS (ESI) m/e 1229.5 (M−H)⁻.

### 2.168.2 2-{[({2-[(3-{[4-(6-{8-[(1,3-benzothiazol-2-yl) carbamoyl]-3,4-dihydroisoquinolin-2(1H)-yl}-2-carboxypyridin-3-yl)-5-methyl-1H-pyrazol-1-yl]methyl}-5,7-dimethyltricyclo[3.3.1.1^{3,7}]decan-1-yl)oxy]ethyl}[(3S)-3,4-dihydroxybutyl]carbamoyl)oxy]methyl}-5-{4-[2-(2,5-dioxo-2,5-dihydro-1H-pyrrol-1-yl)acetamido]butyl}phenyl Beta-D-Glucopyranosiduronic Acid

Example 2.168.2 was prepared by substituting Example 2.168.1 for Example 2.49.1 in Example 2.54. ¹H NMR (501 MHz, dimethyl sulfoxide-d₆) δ 8.07 (s, 1H), 8.01 (dt, 1H), 7.77 (dt, 1H), 7.63-7.57 (m, 1H), 7.51-7.39 (m, 3H), 7.38-7.31 (m, 2H), 7.26 (s, 1H), 7.16 (d, 1H), 7.05 (s, 2H), 6.93 (d, 2H), 6.84-6.80 (m, 1H), 5.14-4.98 (m, 3H), 4.94 (s, 2H), 3.79 (d, 2H), 3.48-3.19 (m, 10H), 3.08-2.96 (m, 4H), 2.52 (s, 4H), 2.07 (s, 2H), 1.77-0.72 (m, 14H). MS (ESI) m/e 1366.5 (M−H)⁻.

### 2.169 Synthesis of 6-{8-[(1,3-benzothiazol-2-yl) carbamoyl]-3,4-dihydroisoquinolin-2(1H)-yl}-3-{1-[(3-{2-[{[(4-{[(2S)-5-(carbamoylamino)-2-{[(2S)-2-{[6-(2,5-dioxo-2,5-dihydro-1H-pyrrol-1-yl)hexanoyl]amino}-3-methylbutanoyl]amino}pentanoyl]amino}phenyl)methoxy]carbonyl}(2-sulfoethyl)amino]acetamido}-5,7-dimethyltricyclo[3.3.1.1^{3,7}]decan-1-yl)methyl]-5-methyl-1H-pyrazol-4-yl}pyridine-2-carboxylic Acid

The title compound was prepared as described in Example 2.54, replacing Example 2.49.1 with Example 1.89.12. ¹H NMR (501 MHz, dimethyl sulfoxide-d₆) δ ppm 9.95 (d, 1H), 8.10-7.96 (m, 1H), 7.75 (t, 2H), 7.57 (dd, 3H), 7.51-7.18 (m, 8H), 6.95 (d, 3H), 6.92 (s, OH), 5.03-4.86 (m, 4H), 4.36 (d, 1H), 3.85 (t, 2H), 3.78-3.67 (m, 4H), 3.42 (s, 2H), 3.33 (t, 2H), 3.04-2.86 (m, 4H), 2.63 (d, 2H), 2.13 (dd, 1H), 2.07 (s, 3H), 1.98-1.87 (m, OH), 1.71-1.23 (m, 10H), 1.24-0.85 (m, 6H), 0.78 (t, 11H). MS (ESI) m/e 1463.5 (M−H)⁻.

### 2.170 Synthesis of N-[6-(2,5-dioxo-2,5-dihydro-1H-pyrrol-1-yl)hexanoyl]-L-valyl-N-{4-[({[2-({3-[(4-{6-[8-(1,3-benzothiazol-2-ylcarbamoyl)-3,4-dihydroisoquinolin-2(1H)-yl]-2-carboxypyridin-3-yl}-5-methyl-1H-pyrazol-1-yl)methyl]-5,7-dimethyltricyclo[3.3.1.1^{3,7}]dec-1-yl}sulfanyl)ethyl](2-sulfoethyl)carbamoyl}oxy)methyl]phenyl}-N⁵-carbamoyl-L-ornithinamide

The title compound was prepared by substituting Example 1.90.11 for Example 1.2.9 in Example 2.1. ¹H NMR (500 MHz, dimethyl sulfoxide-d₆) δ ppm 10.0 (s, 1H), 8.08 (br s, 1H), 8.03 (d, 1H), 7.81 (br s, 1H) 7.78 (d, 1H), 7.60 (m, 3H) 7.52 (t, 1H), 7.47 (t, 1H), 7.43 (d, 1H), 7.37 (d, 1H), 7.34 (d, 1H) 7.32 (s, 1H), 7.28 (d, 2H), 6.99 (s, 1H), 6.96 (d, 2H), 5.00 (s, 2H), 4.96 (s, 2H), 4.39 (m, 1H), 4.18 (m, 2H), 3.88 (m, 2H), 3.82 (s, 1H), 3.77 (s, 1H), 3.46 (br m, 2H), 3.58 (t, 2H), 3.29 (v br m, 2H), 3.01 (br m, 3H), 2.95 (br m, 1H), 2.47 (m, 2H), 2.61 (br m, 2H) 2.16 (m, 1H), 2.10 (m, 4H), 1.96 (br m, 1H), 1.69 (v br m, 1H), 1.59 (v br m, 1H), 1.53-1.40 (m, 7H), 1.39-1.22 (m, 5H), 1.17 (m, 3H), 1.13-0.88 (m, 6H), 0.87-0.77 (m, 9H), 0.75 (s, 3H). MS (ESI) m/e 1466.5 (M−H)⁻.

### 2.171 Synthesis of N-[6-(2,5-dioxo-2,5-dihydro-1H-pyrrol-1-yl)hexanoyl]-L-valyl-N-[4-({[(3-{3-[(4-{6-[8-(1,3-benzothiazol-2-ylcarbamoyl)-3,4-dihydroisoquinolin-2(1H)-yl]-2-carboxypyridin-3-yl}-5-methyl-1H-pyrazol-1-yl)methyl]-5,7-dimethyltricyclo[3.3.1.1^{3,7}]dec-1-yl}propyl)(2-sulfoethyl)carbamoyl]oxy}methyl)phenyl]-N⁵-carbamoyl-L-ornithinamide

The title compound was prepared as described in Example 2.1, replacing Example 1.2.9 with Example 1.91.13. ¹H NMR (501 MHz, DMSO-d₆) δ ppm 12.83 (s, 1H), 9.96 (s, 1H), 8.03 (t, 2H), 7.77 (d, 2H), 7.64-7.52 (m, 3H), 7.45 (ddd, 3H), 7.34 (td, 2H), 7.29-7.21 (m, 3H), 7.03-6.91 (m, 3H), 4.95 (d, 4H), 4.37 (q, 1H), 4.17 (s, 1H), 3.86 (t, 2H), 3.45-3.29 (m, 4H), 3.10 (t, 2H), 2.95 (dt, 4H), 2.61 (q, 2H), 2.15 (td, 2H), 2.07 (s, 3H), 2.00-1.89 (m, 1H), 1.74-1.24 (m, 10H), 1.25-0.87 (m, 13H), 0.88-0.70 (m, 12H). MS (ESI) m/e 1450.2 (M+H)⁺.

### 2.172 Synthesis of 2-{[({2-[(3-{[4-(6-{8-[(1,3-benzothiazol-2-yl) carbamoyl]-3,4-dihydroisoquinolin-2(1H)-yl}-2-carboxypyridin-3-yl)-5-methyl-1H-pyrazol-1-yl]methyl}-5,7-dimethyltricyclo[3.3.1.1^{3,7}]decan-1-yl)oxy]ethyl}[(3S)-3,4-dihydroxybutyl]carbamoyl)oxy]methyl}-5-[4-(2-{(3S,5S)-3-(2,5-dioxo-2,5-dihydro-1H-pyrrol-1-yl)-2-oxo-5-[(2-sulfoethoxy)methyl]pyrrolidin-1-yl}acetamido)butyl]phenyl b-D-glucopyranosiduronic Acid

The title compound was prepared as described in Example 2.119.17, replacing Example 2.168.1 for Example 2.119.16. ¹H NMR (400 MHz, dimethyl sulfoxide-d₆) δ ppm 8.03 (d, 1H), 7.84 (br t, 1H), 7.78 (d, 1H), 7.61 (d, 1H), 7.50 (br d, 1H), 7.45 (dd, 1H), 7.43 (d, 1H), 7.36 (m, 2H), 7.29 (s, 1H), 7.17 (br m, 1H), 7.06 (s, 2H), 6.95 (m, 2H), 6.85 (d, 1H), 5.08 (s, 2H), 5.02 (d, 1H), 4.96 (s, 2H), 4.70 (t, 1H), 4.06 (d, 2H), 3.88 (m, 4H), 3.81 (m, 2H), 3.73 (br m, 1H), 3.62 (m, 2H), 3.47 (br m, 4H), 3.40 (m, 4H), 3.35 (m, 2H), 3.29 (m, 4H), 3.07 (m, 2H), 3.00 (t, 2H), 2.73 (m, 2H), 2.54 (m, 2H), 2.36 (br m, 1H), 2.09 (s, 3H), 1.83 (m, 1H), 1.71 (br m, 1H), 1.55 (br m, 2H), 1.40 (br m, 5H), 1.24 (br m, 4H), 1.10 (br m, 5H), 0.94 (br m, 1H), 0.83, 0.81 (both s, total 6H). MS (ESI) m/e 1587.5 (M−H)⁻.

### 2.173 Synthesis of 2,6-anhydro-8-[2-({[{2-[(3-{[4-(6-{8-[(1,3-benzothiazol-2-yl) carbamoyl]-3,4-dihydroisoquinolin-2 (1H)-yl}-2-carboxypyridin-3-yl)-5-methyl-1H-pyrazol-1-yl]methyl}-5,7-dimethyltricyclo[3.3.1.1^{3,7}]decan-1-yl)oxy]ethyl}(2-sulfoethyl)carbamoyl]oxy}methyl)-5-{[N-({(3R,5S)-3-(2,5-dioxo-2,5-dihydro-1H-pyrrol-1-yl)-2-oxo-5-[(2-sulfoethoxy)methyl]pyrrolidin-1-yl}acetyl)-L-valyl-L-alanyl]amino}phenyl]-7,8-dideoxy-L-glycero-L-gulo-octonic Acid

### 2.173.1 (3R,6R,7aS)-6-azido-3-phenyltetrahydropyrrolo[1,2-c]oxazol-5(3H)-one

The title compound was prepared by substituting Example 2.119.3 for Example 2.119.2 in Example 2.119.4. MS (DCI) m/e 262.0 (M+NH₄)⁺.

### 2.173.2 (3R,6R,7aS)-6-amino-3-phenyltetrahydropyrrolo[1,2-c]oxazol-5(3H)-one

The title compound was prepared by substituting Example 2.173.1 for Example 2.119.4 in Example 2.119.5. MS (DCI) m/e 219.0 (M+H)⁺.

### 2173.3 (3R,6R,7aS)-6-(dibenzylamino)-3-phenyltetrahydropyrrolo[1,2-c]oxazol-5(3H)-one

The title compound was prepared by substituting Example 2.173.2 for Example 2.119.5 in Example 2.119.6. MS (DCI) m/e 399.1 (M+H)⁺.

### 2.173.4 (3R,5S)-3-(dibenzylamino)-5-(hydroxymethyl)pyrrolidin-2-one

The title compound was prepared by substituting Example 2.173.3 for Example 2.119.6 in Example 2.119.7, with the exception that the reaction was heated to 65° C. for one day rather than 6 days. MS (DCI) m/e 311.1 (M+H)+.

### 2.173.5 (3R,5S)-5-(((tert-butyldimethylsilyl)oxy)methyl)-3-(dibenzylamino)pyrrolidin-2-one

The title compound was prepared by substituting Example 2.173.4 for Example 2.119.7 in Example 2.119.8. The title compound was carried on to the next step without purification. MS (DCI) m/e 425.2 (M+H)⁺.

### 2.173.6 tert-butyl 2-((3R,5S)-5-(((tert-butyldimethylsilyl)oxy)methyl)-3-(dibenzylamino)-2-oxopyrrolidin-1-yl)acetate

The title compound was prepared by substituting Example 2.173.5 for Example 2.119.8 in Example 2.119.9. The title compound was carried on to the next step without purification. MS (DCI) m/e 539.3 (M+H)⁺.

### 2.173.7 tert-butyl 2-((3R,5S)-3-(dibenzylamino)-5-(hydroxymethyl)-2-oxopyrrolidin-1-yl)acetate

The title compound was prepared by substituting Example 2.173.6 for Example 2.119.9 in Example 2.119.10. MS (DCI) m/e 425.2 (M+H)⁺.

### 2.173.8 tert-butyl 2-((3R,5S)-5-((2-((4-((tert-butyldiphenylsilyl)oxy)-2,2-dimethylbutoxy)sulfonyl)ethoxy)methyl)-3-(dibenzylamino)-2-oxopyrrolidin-1-yl)acetate

The title compound was prepared by substituting Example 2.173.7 for Example 2.119.10 in Example 2.119.11.

### 2.173.9 tert-butyl(S)-2-(2-((2-((4-((tert-butyldiphenylsilyl)oxy)-2,2-dimethylbutoxy)sulfonyl)ethoxy)methyl)-5-oxopyrrolidin-1-yl)acetate

The title compound was prepared by substituting Example 2.173.8 for Example 2.119.11 in Example 2.119.12. MS (ESI) m/e 691.1 (M+H)⁺.

### 2.173.10 4-(((3R,5S)-1-(2-(tert-butoxy)-2-oxoethyl)-5-((2-((4-((tert-butyldiphenylsilyl)oxy)-2,2-dimethylbutoxy)sulfonyl)ethoxy)methyl)-2-oxopyrrolidin-3-yl)amino)-4-oxobut-2-enoic Acid

The title compound was prepared by substituting Example 2.173.9 for Example 2.119.12 in Example 2.119.13. MS (ESI) m/e 789.0 (M+H)⁺.

### 2.173.11 tert-butyl 2-((3R,5S)-5-((2-((4-((tert-butyldiphenylsilyl)oxy)-2,2-dimethylbutoxy)sulfonyl)ethoxy)methyl)-3-(2,5-dioxo-2,5-dihydro-1H-pyrrol-1-yl)-2-oxopyrrolidin-1-yl)acetate

The title compound was prepared by substituting Example 2173.10 for Example 2.119.13 in Example 2.119.14.

### 2.173.12 2-((3R,5S)-3-(2,5-dioxo-2,5-dihydro-1H-pyrrol-1-yl)-2-oxo-5-((2-sulfoethoxy)methyl)pyrrolidin-1-yl)acetic Acid

The title compound was prepared by substituting Example 2.173.11 for Example 2.119.14 in Example 2.119.15. MS (ESI) m/e 377.0 (M+H)⁺.

### 2.173.13 2,6-anhydro-8-[2-({[{2-[(3-{[4-(6-{8-[(1,3-benzothiazol-2-yl) carbamoyl]-3,4-dihydroisoquinolin-2(1H)-yl}-2-carboxypyridin-3-yl)-5-methyl-1H-pyrazol-1-yl]methyl}-5,7-dimethyltricyclo[3.3.1.1^{3,7}]decan-1-yl)oxy]ethyl}(2-sulfoethyl)carbamoyl]oxy}methyl)-5-{[N-({(3R,5S)-3-(2,5-dioxo-2,5-dihydro-1H-pyrrol-1-yl)-2-oxo-5-[(2-sulfoethoxy)methyl]pyrrolidin-1-yl}acetyl)-L-valyl-L-alanyl]amino}phenyl]-7,8-dideoxy-L-glycero-L-gulo-octonic Acid

The title compound was prepared by substituting Example 2.123.20 for Example 2.119.16 and Example 2.173.12 for Example 2.119.15 in Example 2.119.17. ¹H NMR (400 MHz, dimethyl sulfoxide-d₆) δ ppm 9.94 (d, 1H), 8.28 (br d, 1H), 8.01 (d, 2H), 7.77 (d, 1H), 7.59 (d, 1H), 7.53 (d, 1H), 7.43 (m, 4H), 7.34 (m, 3H), 7.19 (d, 1H), 7.06 (s, 2H), 6.96 (d, 1H), 4.99 (m, 2H), 4.95 (s, 2H), 4.78 (t, 1H), 4.36 (t, 1H), 4.19 (br m, 1H), 4.16 (d, 1H), 3.98 (d, 1H), 3.87 (br t, 2H), 3.81 (br d, 2H), 3.73 (brm, 1H), 3.63 (t, 2H), 3.53 (m, 2H), 3.44 (m, 4H), 3.31 (t, 2H), 3.21 (br m, 2H), 3.17 (m, 2H), 3.00 (m, 2H), 2.92 (br m, 1H), 2.75 (m, 3H), 2.65 (br m, 3H), 2.35 (br m, 1H), 2.16 (m, 1H), 2.07 (s, 3H), 1.98 (br m, 2H), 1.55 (br m, 1H), 1.34 (br m, 1H), 1.26 (br m, 6H), 1.09 (br m, 7H), 0.93 (br m, 1H), 0.87, 0.83, 0.79 (all d, total 12H). MS (ESI) m/e 1733.3 (M−H)⁻.

### 2.174 Synthesis of 2,6-anhydro-8-{2-({[{2-[(3-{[4-(6-{8-[(1,3-benzothiazol-2-yl) carbamoyl]-3,4-dihydroisoquinolin-2(1H)-yl}-2-carboxypyridin-3-yl)-5-methyl-1H-pyrazol-1-yl]methyl}-5,7-dimethyltricyclo[3.3.1.1^{3,7}]decan-1-yl)oxy]ethyl}(2-sulfoethyl)carbamoyl]oxy}methyl)-5-[(N-{[(3R,5S)-3-(2,5-dioxo-2,5-dihydro-1H-pyrrol-1-yl)-2-oxo-5-(41-oxo-2,5,8,11,14,17,20,23,26,29,32,35,38-tridecaoxa-42-azatritetracontan-43-yl)pyrrolidin-1-yl]acetyl}-L-valyl-L-alanyl)amino]phenyl}-7,8-dideoxy-L-glycero-L-gulo-octonic Acid

### 2.174.1 tert-butyl [(3R,5S)-5-{[bis(tert-butoxycarbonyl)amino]methyl}-3-(dibenzylamino)-2-oxopyrrolidin-1-yl]acetate

To a cold (0° C.) solution of Example 2.173.7 (1.6 g) in dichloromethane (15 mL) was added triethylamine (0.70 mL) and methanesulfonyl chloride (0.39 mL) dropwise. The ice-bath was removed, and the reaction was stirred at room temperature for two hours. The reaction was quenched by the addition of saturated aqueous sodium bicarbonate solution. The layers were separated, and the organic layer was washed with brine. The combined aqueous layers were back-extracted with dichloromethane. The combined organic layers were dried with anhydrous sodium sulfate, filtered and concentrated under reduced pressure to give the intermediate mesylate (1.9 g). The residue was dissolved in acetonitrile (15 mL), and di-tert-butyl-iminodicarboxylate (1.0 g) and cesium carbonate (2.4 g) were added. The reaction was heated to reflux under nitrogen for one day. The reaction was cooled and quenched by the addition of water and diethyl ether. The layers were separated, and the organic was washed with brine. The combined aqueous layers were back-extracted with diethyl ether. The combined organic layers were dried with anhydrous sodium sulfate, filtered and concentrated under reduced pressure. The residue was purified by silica gel chromatography, eluting with 20% ethyl acetate in heptanes, to give the title compound. MS (DCI) m/e 624.3 (M+H)⁺.

### 2.174.2 tert-butyl [(3R,5S)-3-amino-5-{[bis(tert-butoxycarbonyl)amino]methyl}-2-oxopyrrolidin-1-yl]acetate

To a solution of Example 2.174.1 (1.0 g) in ethyl acetate (6 mL) and methanol (18 mL) was added palladium hydroxide on carbon (100 mg, 20% by weight). The reaction was stirred at room temperature under a hydrogen balloon for one day. The reaction was filtered through diatomaceous earth, eluting with ethyl acetate. The filtrate was concentrated under reduced pressure, dissolved in dichloromethane (10 mL) and filtered through a syringe-tip Teflon 40 micron filter. The filtrate was concentrated under reduced pressure to give the title compound. MS (DCI) m/e 444.1 (M+H)⁺.

### 2.174.3 4-{[(3R,5S)-5-{[bis(tert-butoxycarbonyl)amino]methyl}-1-(2-tert-butoxy-2-oxoethyl)-2-oxopyrrolidin-3-yl]amino}-4-oxobut-2-enoic Acid

The title compound was prepared by substituting Example 2.174.2 for Example 2.119.12 in Example 2.119.13. MS (ESI) m/e 540.2 (M−H)⁻.

### 2.174.4 tert-butyl [(3R,5S)-5-{[bis(tert-butoxycarbonyl)amino]methyl}-3-(2,5-dioxo-2,5-dihydro-1H-pyrrol-1-yl)-2-oxopyrrolidin-1-yl]acetate

The title compound was prepared by substituting Example 2.174.3 for Example 2.119.13 in Example 2.119.14. MS (DCI) m/e 541.1 (M+NH₄)⁺.

### 2.174.5 2-((3R,5S)-5-(aminomethyl)-3-(2,5-dioxo-2,5-dihydro-1H-pyrrol-1-yl)-2-oxopyrrolidin-1-yl)acetic Acid

To a solution of Example 2.174.4 (284 mg) in dichloromethane (10 mL) was added trifluoroacetic acid (5 mL). The reaction was stirred at room temperature for two hours and was concentrated under reduced pressure. The residue was dissolved in water/acetonitrile 7/3 (5 mL), frozen and lyophilized to provide the title compound, which was used in the subsequent step without further purification. MS (ESI) m/e 266.1 (M−H)⁻.

### 2.174.6 2-((3R,5S)-3-(2,5-dioxo-2,5-dihydro-1H-pyrrol-1-yl)-2-oxo-5-(41-oxo-2,5,8,11,14,17,20,23,26,29,32,35,38-tridecaoxa-42-azatritetracontan-43-yl)pyrrolidin-1-yl)acetic Acid

To a solution of 2,5,8,11,14,17,20,23,26,29,32,35,38-tridecaoxahentetracontan-41-oic acid (160 mg) in N,N-dimethylformamide (1.0 mL) was added O-(7-azabenzotriazol-1-yl)-N,N,N′,N′-tetramethyluronium hexafluorophosphate (85 mg) and N,N-diisopropylethylamine (130 μL). The reaction mixture was stirred for three minutes at room temperature, and a solution of Example 2.174.5 (70 mg) and N,N-diisopropylethylamine (130 μL) in N,N-dimethylformamide (1.0 mL) was added. The reaction was stirred at room temperature for one hour and diluted with N,N-dimethylformamide/water 1/1 (3.5 mL). The solution was purified by reverse phase HPLC on a Gilson system (C18 column), eluting with 20-70% acetonitrile in 0.1% TFA water, to provide the title compound. MS (ESI) m/e 880.4 (M−H)⁻.

### 2.174.7 2,6-anhydro-8-{2-({[{2-[(3-{[4-(6-{8-[(1,3-benzothiazol-2-yl) carbamoyl]-3,4-dihydroisoquinolin-2(1H)-yl}-2-carboxypyridin-3-yl)-5-methyl-1H-pyrazol-1-yl]methyl}-5,7-dimethyltricyclo[3.3.1.1^{3,7}]decan-1-yl)oxy]ethyl}(2-sulfoethyl)carbamoyl]oxy}methyl)-5-[(N-{[(3R,5S)-3-(2,5-dioxo-2,5-dihydro-1H-pyrrol-1-yl)-2-oxo-5-(41-oxo-2,5,8,11,14,17,20,23,26,29,32,35,38-tridecaoxa-42-azatritetracontan-43-yl)pyrrolidin-1-yl]acetyl}-L-valyl-L-alanyl)amino]phenyl}-7,8-dideoxy-L-glycero-L-gulo-octonic Acid

The title compound was prepared by substituting Example 2.174.6 for Example 2.119.15 and Example 2.123.20 for Example 2.119.16 in Example 2.119.17 ¹H NMR (500 MHz, dimethyl sulfoxide-d₆) δ ppm 9.93 (br d, 1H), 8.28 (d, 1H), 8.03 (d, 1H), 8.02 (br s, 1H), 7.91 (br d, 1H), 7.79 (d, 1H), 7.61 (d, 1H), 7.51 (br d, 1H), 7.49-7.42 (m, 3H), 7.40 (br d, 1H), 7.36 (m, 2H), 7.28 (s, 1H), 7.22 (d, 1H), 7.06 (s, 2H), 6.95 (d, 1H), 5.00 (br d, 2H), 4.95 (s, 2H), 4.70 (t, 1H), 4.39 (m, 1H), 4.28 (m, 1H), 4.00 (dd, 2H), 3.88 (br m, 2H), 3.85 (br m, 1H), 3.80 (br m, 2H), 3.62 (t, 2H), 3.50 (s, 44H), 3.48 (d, 4H), 3.43 (br m, 2H), 3.34 (br m, 2H), 3.23 (s, 3H), 3.21 (v br m, 2H), 3.14 (t, 2H), 3.10 (v br m, 1H), 3.00 (t, 2H), 2.94 (br m, 1H), 2.76 (v br m, 1H), 2.64 (v br m, 3H), 2.34 (br t, 2H), 2.32 (m, 1H), 2.17 (m, 1H), 2.09 (br d, 3H), 2.00 (br m, 1H), 1.56 (br m, 1H), 1.39-1.19 (br m, 8H), 1.19-0.92 (br m, 8H), 0.88 (br d, 3H), 0.87 (br m, 1H), 0.82 (br d, 6H), 0.79 (br s, 3H). MS (ESI) m/e 1119.2 [(M−2H)/2]⁻.

### 2.175 Synthesis of (6S)-2,6-anhydro-6-(2-{2-[({[2-({3-[(4-{6-[8-(1,3-benzothiazol-2-ylcarbamoyl)-3,4-dihydroisoquinolin-2(1H)-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl][(3S)-3,4-dihydroxybutyl]carbamoyl}oxy)methyl]-5-{(N-[(2,5-dioxo-2,5-dihydro-1H-pyrrol-1-yl)acetyl]-N-(2,5,8,11,14,17,20,23,26,29,32-undecaoxatetratriacontan-34-yl)-b-alanyl-L-valyl-L-alanyl}amino)phenyl}ethyl)-L-gluconic Acid

The title compound was prepared using the procedure in Example 2.147.4, replacing Example 2.141.4 with Example 2.167.1. MS (ESI) m/e 1033.4 (M+2H)²⁺.

### 2.176 Synthesis of (6S)-2,6-anhydro-6-(2-{2-[({[2-({3-[(4-{6-[8-(1,3-benzothiazol-2-ylcarbamoyl)-3,4-dihydroisoquinolin-2(1H)-yl]-2-carboxypyridin-3-yl}-5-methyl-1H-pyrazol-1-yl)methyl]-5,7-dimethyltricyclo[3.3.1.1^{3,7}]dec-1-yl}oxy)ethyl][(3S)-3,4-dihydroxybutyl]carbamoyl}oxy)methyl]-5-({N-[(2,5-dioxo-2,5-dihydro-1H-pyrrol-1-yl)acetyl]-N-[2-(2-sulfoethoxy)ethyl]-b-alanyl-L-valyl-L-alanyl}amino)phenyl}ethyl)-L-gluconic Acid

The title compound was prepared using the procedure in Example 2.160.7, replacing Example 2.154.1 with Example 2.167.1. MS (ESI) m/e 859.4 (M+2H)²⁺.

### Example 2. Synthesis of Exemplary Bcl-xL Inhibitory ADCs

Exemplary ADCs were synthesized using one of four exemplary methods, described below. Table 1 correlates which method was used to synthesize each exemplary ADC.

Method A. A solution of TCEP (10 mM, 0.017 mL) was added to a solution of antibody (10 mg/mL, 1 mL) preheated to 37° C. The reaction mixture was kept at 37° C. for 1 hour. The solution of reduced antibody was added to a solution of linker-warhead payload (3.3 mM, 0.160 mL in DMSO) and gently mixed for 30 minutes. The reaction solution was loaded onto a desalting column (PD10, washed with DPBS 3× before use), followed by DPBS (1.6 mL) and eluted with additional DPBS (3 mL). The purified ADC solution was filtered through a 0.2 micron, low protein-binding 13 mm syringe-filter and stored at 4° C.

Method B. A solution of TCEP (10 mM, 0.017 mL) was added to the solution of antibody (10 mg/mL, 1 mL) preheated to 37° C. The reaction mixture was kept at 37° C. for 1 hour. The solution of reduced antibody was adjusted to pH=8 by adding boric buffer (0.05 mL, 0.5 M, pH8), added to a solution of linker-warhead payload (3.3 mM, 0.160 mL in DMSO) and gently mixed for 4 hours. The reaction solution was loaded onto a desalting column (PD10, washed with DPBS 3× before use), followed by DPBS (1.6 mL) and eluted with additional DPBS (3 mL). The purified ADC solution was filtered through a 0.2 micron, low protein-binding 13 mm syringe-filter and stored at 4° C.

Method Q. Conjugations were performed using a PerkinElmer Janus (part AJL8M01) robotic liquid handling system equipped with an I235/96 tip ModuLar Dispense Technology (MDT), disposable head (part 70243540) containing a gripper arm (part 7400358), and an 8-tip Varispan pipetting arm (part 7002357) on an expanded deck. The PerkinElmer Janus system was controlled using the WinPREP version 4.8.3.315 Software.

A Pall Filter plate 5052 was prewet with 100 μL 1×DPBS using the MDT. Vacuum was applied to the filter plate for 10 seconds and was followed by a 5 second vent to remove DPBS from filter plate. A 50% slurry of Protein A resin (GE MabSelect Sure) in DPBS was poured into an 8 well reservoir equipped with a magnetic ball, and the resin was mixed by passing a traveling magnet underneath the reservoir plate. The 8 tip Varispan arm, equipped with 1 mL conductive tips, was used to aspirate the resin (250 μL) and transfer to a 96-well filter plate. A vacuum was applied for 2 cycles to remove most of the buffer. Using the MDT, 150 μL of 1×PBS was aspirated and dispensed to the 96-well filter plate holding the resin. A vacuum was applied, removing the buffer from the resin. The rinse/vacuum cycle was repeated 3 times. A 2 mL, 96-well collection plate was mounted on the Janus deck, and the MDT transferred 450 μL of 5×DPBS to the collection plate for later use. Reduced antibody (2 mg) as a solution in (200 μL) DPBS was prepared as described above for Conditions A and preloaded into a 96 well plate. The solutions of reduced antibody were transferred to the filter plate wells containing the resin, and the mixture was mixed with the MDT by repeated aspiration/dispensation of a 100 μL volume within the well for 45 seconds per cycle. The aspiration/dispensation cycle was repeated for a total of 5 times over the course of 5 minutes. A vacuum was applied to the filter plate for 2 cycles, thereby removing excess antibody. The MDT tips were rinsed with water for 5 cycles (200 μL, 1 mL total volume). The MDT aspirated and dispensed 150 μL of DPBS to the filter plate wells containing resin-bound antibody, and a vacuum was applied for two cycles. The wash and vacuum sequence was repeated two more times. After the last vacuum cycle, 100 μL of 1×DPBS was dispensed to the wells containing the resin-bound antibody. The MDT then collected 30 μL each of 3.3 mM dimethyl sulfoxide solutions of synthons plated in a 96-well format and dispensed it to the filter plate containing resin-bound antibody in DPBS. The wells containing the conjugation mixture were mixed with the MDT by repeated aspiration/dispensation of a 100 μL volume within the well for 45 seconds per cycle. The aspiration/dispensation sequence was repeated for a total of 5 times over the course of 5 minutes. A vacuum was applied for 2 cycles to remove excess synthon to waste. The MDT tips were rinsed with water for 5 cycles (200 μL, 1 mL total volume). The MDT aspirated and dispensed DPBS (150 μL) to the conjugation mixture, and a vacuum was applied for two cycles. The wash and vacuum sequence was repeated two more times. The MDT gripper then moved the filter plate and collar to a holding station. The MDT placed the 2 mL collection plate containing 450 μL of 10×DPBS inside the vacuum manifold. The MDT reassembled the vacuum manifold by placement of the filter plate and collar. The MDT tips were rinsed with water for 5 cycles (200 μL, 1 mL total volume). The MDT aspirated and dispensed 100 μL of IgG Elution Buffer 3.75 (Pierce) to the conjugation mixture. After one minute, a vacuum was applied for 2 cycles, and the eluent was captured in the receiving plate containing 450 μL of 5×DPBS. The aspiration/dispensation sequence was repeated 3 additional times to deliver ADC samples with concentrations in the range of 1.5-2.5 mg/mL at pH 7.4 in DPBS.

Method D. Conjugations were performed using a PerkinElmer Janus (part AJL8M01) robotic liquid handling system equipped with an I235/96 tip ModuLar Dispense Technology (MDT), disposable head (part 70243540) containing a gripper arm (part 7400358), and an 8-tip Varispan pipetting arm (part 7002357) on an expanded deck. The PerkinElmer Janus system was controlled using the WinPREP version 4.8.3.315 Software.

A Pall Filter plate 5052 was prewet with 100 μL 1×DPBS using the MDT. Vacuum was applied to the filter plate for 10 seconds and was followed by a 5 second vent to remove DPBS from filter plate. A 50% slurry of Protein A resin (GE MabSelect Sure) in DPBS was poured into an 8-well reservoir equipped with a magnetic ball, and the resin was mixed by passing a traveling magnet underneath the reservoir plate. The 8 tip Varispan arm, equipped with 1 mL conductive tips, was used to aspirate the resin (250 μL) and transfer to a 96-well filter plate. A vacuum was applied to the filter plate for 2 cycles to remove most of the buffer. The MDT aspirated and dispensed 150 μL of DPBS to the filter plate wells containing the resin. The wash and vacuum sequence was repeated two more times. A 2 mL, 96-well collection plate was mounted on the Janus deck, and the MDT transferred 450 μL of 5×DPBS to the collection plate for later use. Reduced antibody (2 mg) as a solution in (200 μL) DPBS was prepared as described above for Conditions A and dispensed into the 96-well plate. The MDT then collected 30 μL each of 3.3 mM dimethyl sulfoxide solutions of synthons plated in a 96-well format and dispensed it to the plate loaded with reduced antibody in DPBS. The mixture was mixed with the MDT by twice repeated aspiration/dispensation of a 100 μL volume within the well. After five minutes, the conjugation reaction mixture (230 μL) was transferred to the 96-well filter plate containing the resin. The wells containing the conjugation mixture and resin were mixed with the MDT by repeated aspiration/dispensation of a 100 μL volume within the well for 45 seconds per cycle. The aspiration/dispensation sequence was repeated for a total of 5 times over the course of 5 minutes. A vacuum was applied for 2 cycles to remove excess synthon and protein to waste. The MDT tips were rinsed with water for 5 cycles (200 μL, 1 mL total volume). The MDT aspirated and dispensed DPBS (150 μL) to the conjugation mixture, and a vacuum was applied for two cycles. The wash and vacuum sequence was repeated two more times. The MDT gripper then moved the filter plate and collar to a holding station. The MDT placed the 2 mL collection plate containing 450 μL of 10×DPBS inside the vacuum manifold. The MDT reassembled the vacuum manifold by placement of the filter plate and collar. The MDT tips were rinsed with water for 5 cycles (200 μL, 1 mL total volume). The MDT aspirated and dispensed 100 μL of IgG Elution Buffer 3.75 (P) to the conjugation mixture. After one minute, a vacuum was applied for 2 cycles, and the eluent was captured in the receiving plate containing 450 μL of 5×DPBS. The aspiration/dispensation sequence was repeated 3 additional times to deliver ADCsamples with concentrations in the range of 1.5-2.5 mg/mL at pH 7.4 in DPBS.

Method E. A solution of TCEP (10 mM, 0.017 mL) was added to the solution of antibody (10 mg/mL, 1 mL) at room temperature. The reaction mixture was heated to 37° C. for 75 minutes. The solution of reduced antibody cooled to room temperature and was added to a solution of synthon (10 mM, 0.040 mL in DMSO) followed by addition of boric buffer (0.1 mL, 1M, pH 8). The reaction solution was let to stand for 3 days at room temperature, loaded onto a desalting column (PD10, washed with DPBS 3×5 mL before use), followed by DPBS (1.6 mL) and eluted with additional DPBS (3 mL). The purified ADC solution was filtered through a 0.2 micron, low protein-binding 13 mm syringe-filter and stored at 4 C.

Method F. Conjugations were performed using a Tecan Freedom Evo robotic liquid handling system.

The solution of antibody (10 mg/mL) was preheated to 37° C. and aliquoted to a heated 96 deep-well plate in amounts of 3 mg per well (0.3 mL) and kept at 37 C. A solution of TCEP (1 mM, 0.051 mL/well) was added to antibodies, and the reaction mixture was kept at 37° C. for 75 minutes. The solution of reduced antibody was transferred to an unheated 96 deep-well plate. Corresponding solutions of synthons (5 mM, 0.024 mL in DMSO) were added to the wells with reduced antibodies and treated for 15 minutes. The reaction solutions were loaded onto a platform (8×12) of desalting columns (NAP5, washed with DPBS 4× before use), followed by DPBS (0.3 mL) and eluted with additional DPBS (0.8 mL). The purified ADC solutions were further aliquoted for analytics and stored at 4° C.

Method G. Conjugations were performed using a Tecan Freedom Evo robotic liquid handling system.

The solution of antibody (10 mg/mL) was preheated to 37° C. and aliquoted onto a heated 96 deep-well plate in amounts of 3 mg per well (0.3 mL) and kept at 37 C. A solution of TCEP (1 mM, 0.051 mL/well) was added to antibodies, and the reaction mixture was kept at 37° C. for 75 minutes. The solutions of reduced antibody were transferred to an unheated 96 deep-well plate. Corresponding solutions of synthons (5 mM, 0.024 mL/well in DMSO) were added to the wells with reduced antibodies followed by addition of boric buffer (pH=8, 0.03 mL/well) and treated for 3 days. The reaction solutions were loaded onto a platform (8×12) of desalting columns (NAP5, washed with DPBS 4× before use), followed by DPBS (0.3 mL) and eluted with additional DPBS (0.8 mL). The purified ADC solutions were further aliquoted for analytics and stored at 4° C.

Method H. A solution of TCEP (10 mM, 0.17 mL) was added to the solution of antibody (10 mg/mL, 10 mL) at room temperature. The reaction mixture was heated to 37° C. for 75 minutes. The solution of synthon (10 mM, 0.40 mL in DMSO) was added to a solution of reduced antibody cooled to room temperature. The reaction solution was let to stand for 30 minutes at room temperature. The solution of ADC was treated with saturated ammonium sulfate solution (˜2-2.5 mL) until a slightly cloudy solution formed. This solution was loaded onto butyl sepharose column (5 mL of butyl sepharose) equilibrated with 30% phase B in phase A (phase A: 1.5 M ammonium sulfate, 25 mM phosphate; phase B: 25 mM phosphate, 25% isopropanol v/v). Individual fractions with DAR2 (also referred to as “E2”) and DAR4 (also referred to as “E4”) eluted upon applying gradient A/B up to 75% phase B. Each ADC solution was concentrated and buffer switched using centrifuge concentrators or TFF for larger scales. The purified ADC solutions were filtered through a 0.2 micron, low protein-binding 13 mm syringe-filter and stored at 4 C.

Method I. A solution of TCEP (10 mM, 0.17 mL) was added to the solution of antibody (10 mg/mL, 10 mL) at room temperature. The reaction mixture was heated to 37° C. for 75 minutes. The solution of synthon (10 mM, 0.40 mL in DMSO) was added to a solution of reduced antibody cooled to room temperature. The reaction solution was let to stand for 30 minutes at room temperature. The solution of ADC was treated with saturated ammonium sulfate solution (˜2-2.5 mL) until a slightly cloudy solution formed. This solution was loaded onto a butyl sepharose column (5 mL of butyl sepharose) equilibrated with 30% phase B in Phase A (phase A: 1.5 M ammonium sulfate, 25 mM phosphate; phase B: 25 mM phosphate, 25% isopropanol v/v). Individual fractions with DAR2 (also referred to as “E2”) and DAR 4 (also referred to as “E4”) eluted upon applying a gradient A/B up to 75% phase B. Each ADC solution was concentrated and buffer switched using centrifuge concentrators or TFF for larger scales. The ADC solutions were treated with boric buffer (0.1 mL, 1M, pH8). The reaction solution was let stand for 3 days at room temperature, then loaded onto a desalting column (PD10, washed with DPBS 3×5 mL before use), followed by DPBS (1.6 mL) and eluted with additional DPBS (3 mL). The purified ADC solution was filtered through a 0.2 micron, low protein-binding 13 mm syringe-filter and stored at 4 C.

Table 1, below, indicates which exemplary ADCs were synthesized via which exemplary method. The monoclonal antibody to EpCAM referred to as EpCAM(ING-1) is described in Studnicka et al., 1994, Protein Engineering, 7:805-814 and Ammons et al., 2003, Neoplasia 5:146-154. The NCAM-1 antibody referred to as N901 is described in Roguska et al., 1994, Proc Natl Acad Sci USA 91:969-973. The EGFR antibody referred to as AB033 is described in WO 2009/134776 (see page 120).

TABLE 1
Synthetic Methods Used to Synthesize Exemplary ADCs

| Appln | | |
|---|---|---|
| Ex. No. | ADC | Method |
| 3.1 | AB033-CZ | A |
| 3.2 | AB033-DH | A |
| 3.4 | AB033-EP | A |
| 3.6 | AB033-EG | A |
| 3.7 | AB033-EH | A |
| 3.8 | AB033-ER | A |
| 3.9 | AB033-ES | A |
| 3.10 | AB033-EQ | A |
| 3.11 | AB033-EU | A |
| 3.12 | AB033-EV | A |
| 3.13 | AB033-EW | A |
| 3.14 | AB033-EX | A |
| 3.15 | AB033-EY | A |
| 3.16 | AB033-EZ | A |
| 3.17 | AB033-FD | A |
| 3.18 | AB033-FS | A |
| 3.19 | AB033-FI | A |
| 3.20 | AB033-FV | A |
| 3.21 | AB033-GC | A |
| 3.22 | AB033-GB | A |
| 3.23 | AB033-FW | A |
| 3.24 | AB033-GD | A |
| 3.25 | AB033-GK | A |
| 3.26 | AB033-GJ | A |
| 3.27 | AB033-GW | A |
| 3.28 | AB033-HF | A |
| 3.29 | AB033-HG | A |
| 3.30 | AB033-HP | A |
| 3.31 | AB033-HR | A |
| 3.32 | AB033-HU | A |
| 3.33 | AB033-HT | A |
| 3.34 | AB033-HV | A, C |
| 3.35 | AB033-HZ | A, C |
| 3.36 | AB033-IA | A, C |
| 3.37 | AB033-IF | A, C |
| 3.38 | AB033-IG | B |
| 3.39 | AB033-IH | A, C |
| 3.40 | AB033-IJ | A |
| 3.41 | AB033-IK | — |
| 3.42 | AB033-IL | A, C |
| 3.43 | AB033-IM | A, C |
| 3.44 | AB033-IO | A, C |
| 3.45 | AB033-IP | A, C |
| 3.46 | AB033-IS | A, C |
| 3.47 | AB033-IU | A, C |
| 3.48 | AB033-IV | A, C |
| 3.49 | AB033-IZ | B |
| 3.50 | AB033-JK | A |
| 3.51 | AB033-JF | A, C |
| 3.52 | AB033-FE | A |
| 3.53 | AB033-GG | A |
| 3.54 | AB033-GM | A |
| 3.55 | AB033-HD | A |
| 3.56 | AB033-HS | A |
| 3.57 | AB033-HW | A, C |
| 3.58 | AB033-HX | A, C |
| 3.59 | AB033-HY | A, C |
| 3.60 | AB033-IB | A, C |
| 3.61 | AB033-IE | A, C |
| 3.62 | AB033-II | A, C |
| 3.63 | AB033-JJ | A, D |
| 3.64 | AB033-IW | A, C |
| 3.65 | AB033-IY | A, C |
| 3.66 | AB033-JA | A, C |
| 3.74 | AB033-FA | A |
| 3.75 | AB033-FJ | A |
| 3.76 | AB033-FK | A |
| 3.77 | AB033-FR | A |
| 3.78 | AB033-JE | A |
| 3.79 | AB033-JL | A |
| 3.80 | AB033-LE | D |
| 3.81 | AB033-LH | B |
| 3.82 | AB033-LJ | D |
| 3.83 | AB033-MA | D |
| 3.84 | AB033-MD | D |
| 3.85 | AB033-MG | D |
| 3.86 | AB033-MS | D |
| 3.87 | AB033-MR | D |
| 3.88 | AB033-MQ | A |
| 3.89 | AB033-MZ | B |
| 3.90 | AB033-NA | A |
| 3.91 | AB033-NB | B |
| 3.92 | AB033-NN | D |
| 3.93 | AB033-NO | D |
| 3.94 | EpCAM (ING-1)-CZ | A |
| 3.96 | EpCAM (ING-1)-FE | A |
| 3.97 | EpCAM (ING-1)-GG | A |
| 3.98 | EpCAM (ING-1)-GM | A |
| 3.99 | EpCAM (ING-1)-HD | A |
| 3.100 | EpCAM (ING-1)-HS | A |
| 3.101 | EpCAM (ING-1)-HW | A |
| 3.102 | EpCAM (ING-1)-HX | A |
| 3.103 | EpCAM (ING-1)-HY | A |
| 3.104 | EpCAM (ING-1)-IB | A |
| 3.105 | EpCAM (ING-1)-IE | A |
| 3.106 | EpCAM (ING-1)-IJ | A |
| 3.107 | EpCAM (ING-1)-IK | A |
| 3.108 | EpCAM (ING-1)-IL | A |
| 3.123 | N901-CZ | A |
| 3.124 | AB033-OK | A |
| 3.125 | AB033-OW | D |
| 3.126 | AB033-PC | D |
| 3.127 | AB033-PI | D |
| 3.128 | AB033-PJ | D |
| 3.129 | AB033-PU | D |
| 3.130 | AB033-PV | D |
| 3.131 | AB033-PW | D |
| 3.132 | AB033-QW | D |
| 3.133 | AB033-RM | D |
| 3.134 | AB033-RR | A |
| 3.135 | AB033-SJ | E |
| 3.136 | AB033-SM | A |
| 3.137 | AB033-SN | A |
| 3.138 | AB033-SS | A |
| 3.139 | AB033-TA | E |
| 3.140 | AB033-TW | G |
| 3.141 | AB033-ST | A |
| 3.142 | AB033-ZL | E |
| 3.143 | AB033-SX | E |
| 3.144 | AB033-SW | E |
| 3.145 | AB033-TV | E |
| 3.146 | AB033-SZ | E |
| 3.147 | AB033-ZM | G |
| 3.148 | AB033-SV | E |
| 3.149 | AB033-SY | A |
| 3.150 | AB033-TK | G |
| 3.151 | AB033-TR | E |
| 3.152 | AB033-TY | E |
| 3.153 | AB033-TX | E |
| 3.154 | AB033-TZ | E |
| 3.155 | AB033-UA | E |
| 3.156 | AB033-UJ | E |
| 3.157 | AB033-UK | E |
| 3.158 | AB033-UU | G |
| 3.159 | AB033-UV | G |
| 3.160 | AB033-UZ | E |
| 3.161 | AB033-VB | E |
| 3.162 | AB033-VC | E |
| 3.163 | AB033-VS | E |
| 3.164 | AB033-VT | E |
| 3.165 | AB033-VY | E |
| 3.166 | AB033-WI | E |
| 3.167 | AB033-WK | E |
| 3.168 | AB033-WP | E |
| 3.169 | AB033-XD | G |
| 3.170 | AB033-XK | G |
| 3.171 | AB033-XL | G |
| 3.172 | AB033-YJ | G |
| 3.173 | AB033-YQ | G |
| 3.174 | AB033-YR | G |
| 3.175 | AB033-YS | G |
| 3.176 | AB033-YY | G |
| 3.177 | AB033-YT | G |
| 3.178 | AB033-YU | G |
| 3.179 | AB033-YV | G |
| 3.180 | AB033-YW | G |
| 3.181 | AB033-ZB | G |
| 3.182 | AB033-ZC | G |
| 3.183 | AB033-ZJ | G |
| 3.184 | AB033-ZE | G |
| 3.185 | AB033-ZW | G |
| 3.186 | AB033-ZX | G |
| 3.187 | AB033-AAA | G |
| 3.188 | AB033-AAD | G |
| 3.189 | AB033-AAE | G |
| 3.190 | AB033-AAF | G |
| 3.191 | AB033-JL (E2) | I |
| 3.192 | AB033-JL (E4) | I |
| 3.193 | AB033-TX (E2) | I |
| 3.194 | AB033-TX (E4) | I |

### Example 3. Exemplary Bcl-xL Inhibitors Bind Bcl-xL

The ability of the exemplary Bcl-xL inhibitors of Examples 1.1 through 1.91 (compounds W2.01-W2.91 respectively) to bind Bcl-xL was demonstrated using the Time Resolved-Fluorescence Resonance Energy Transfer (TR-FRET) Assay. Tb-anti-GST antibody was purchased from Invitrogen (Catalog No. PV4216).

### 3.1. Probe Synthesis

### 3.1.1. Reagents

All reagents were used as obtained from the vendor unless otherwise specified. Peptide synthesis reagents including diisopropylethylamine (DIEA), dichloromethane (DCM), N-methylpyrrolidone (NMP), 2-(1H-benzotriazole-1-yl)-1,1,3,3-tetramethyluronium hexafluorophosphate (HBTU), N-hydroxybenzotriazole (HOBt) and piperidine were obtained from Applied Biosystems, Inc. (ABI), Foster City, CA or American Bioanalytical, Natick, MA.

Preloaded 9-Fluorenylmethyloxycarbonyl (Fmoc)amino acid cartridges (Fmoc-Ala-OH, Fmoc-Cys(Trt)-OH, Fmoc-Asp(tBu)-OH, Fmoc-Glu(tBu)-OH, Fmoc-Phe-OH, Fmoc-Gly-OH, Fmoc-His(Trt)-OH, Fmoc-Ile-OH, Fmoc-Leu-OH, Fmoc-Lys(Boc)-OH, Fmoc-Met-OH, Fmoc-Asn(Trt)-OH, Fmoc-Pro-OH, Fmor-Gln(Trt)-OH, Fmoc-Arg(Pbf)-OH, Fmoc-Ser(tBu)-OH, Fmoc-Thr(tBu)-OH, Fmoc-Val-OH, Fmoc-Trp(Boc)-OH, Fmoc-Tyr(tBu)-OH) were obtained from ABI or Anaspec, San Jose, CA.

The peptide synthesis resin (Fmoc-Rink amide MBHA resin) and Fmoc-Lys(Mtt)-OH were obtained from Novabiochem, San Diego, CA.

Single-isomer 6-carboxyfluorescein succinimidyl ester (6-FAM-NHS) was obtained from Anaspec.

Trifluoroacetic acid (TFA) was obtained from Oakwood Products, West Columbia, SC.

Thioanisole, phenol, triisopropylsilane (TIS), 3,6-dioxa-1,8-octanedithiol (DODT) and isopropanol were obtained from Aldrich Chemical Co., Milwaukee, WI.

Matrix-assisted laser desorption ionization mass-spectra (MALDI-MS) were recorded on an Applied Biosystems Voyager DE-PRO MS).

Electrospray mass-spectra (ESI-MS) were recorded on Finnigan SSQ7000 (Finnigan Corp., San Jose, CA) in both positive and negative ion mode.

### 3.1.2. General Procedure for Solid-Phase Peptide Synthesis (SPPS)

Peptides were synthesized with, at most, 250 μmol preloaded Wang resin/vessel on an ABI 433A peptide synthesizer using 250 μmol scale Fastmoc™ coupling cycles. Preloaded cartridges containing 1 mmol standard Fmoc-amino acids, except for the position of attachment of the fluorophore, where 1 mmol Fmoc-Lys(Mtt)-OH was placed in the cartridge, were used with conductivity feedback monitoring. N-terminal acetylation was accomplished by using 1 mmol acetic acid in a cartridge under standard coupling conditions.

### 3.1.3. Removal of 4-Methyltrityl (Mtt) from Lysine

The resin from the synthesizer was washed thrice with dichloromethane and kept wet. 150 mL of 95:4:1 dichloromethane:triisopropylsilane:trifluoroacetic acid was flowed through the resin bed over 30 minutes. The mixture turned deep yellow then faded to pale yellow. 100 mL of DMF was flowed through the bed over 15 minutes. The resin was then washed thrice with DMF and filtered. Ninhydrin tests showed a strong signal for primary amine.

### 3.1.4. Resin Labeling with 6-Carboxyfluorescein-NHS (6-FAM-NHS)

The resin was treated with 2 equivalents 6-FAM-NHS in 1% DIEA/DMF and stirred or shaken at ambient temperature overnight. When complete, the resin was drained, washed thrice with DMF, thrice with (1× dichloromethane and 1× methanol) and dried to provide an orange resin that was negative by ninhydrin test.

### 3.1.5. General Procedure for Cleavage and Deprotection of Resin-Bound Peptide

Peptides were cleaved from the resin by shaking for 3 hours at ambient temperature in a cleavage cocktail consisting of 80% TFA, 5% water, 5% thioanisole, 5% phenol, 2.5% TIS, and 2.5% EDT (1 mL/0.1 g resin). The resin was removed by filtration and rinsing twice with TFA. The TFA was evaporated from the filtrates, and product was precipitated with ether (10 mL/0.1 g resin), recovered by centrifugation, washed twice with ether (10 mL/0.1 g resin) and dried to give the crude peptide.

### 3.1.6. General Procedure for Purification of Peptides

The crude peptides were purified on a Gilson preparative HPLC system running Unipoint® analysis software (Gilson, Inc., Middleton, WI) on a radial compression column containing two 25×100 mm segments packed with Delta-Pak™ C18 15 μm particles with 100 Å pore size and eluted with one of the gradient methods listed below. One to two milliliters of crude peptide solution (10 mg/mL in 90% DMSO/water) was purified per injection. The peaks containing the product(s) from each run were pooled and lyophilized. All preparative runs were run at 20 mL/min with eluents as buffer A: 0.1% TFA-water and buffer B: acetonitrile.

### 3.1.7. General Procedure for Analytical HPLC

Analytical HPLC was performed on a Hewlett-Packard 1200 series system with a diode-array detector and a Hewlett-Packard 1046A fluorescence detector running HPLC 3D ChemStation software version A.03.04 (Hewlett-Packard. Palo Alto, CA) on a 4.6×250 mm YMC column packed with ODS-AQ 5 μm particles with a 120 Å pore size and eluted with one of the gradient methods listed below after preequilibrating at the starting conditions for 7 minutes. Eluents were buffer A: 0.1% TFA-water and buffer B: acetonitrile. The flow rate for all gradients was 1 mL/min.

### 3.1.8. Synthesis of Probe F-Bak

Peptide probe F-bak, which binds Bcl-xL, was synthesized as described below. Probe F-Bak is acetylated at the N-terminus, amidated at the C-terminus and has the amino acid sequence GQVGRQLAIIGDKINR. It is fluoresceinated at the lysine residue (K) with 6-FAM. Probe F-Bak can be abbreviated as follows: acetyl-GQVGRQLAIIGDK(6-FAM)INR—NH₂.

To make probe F-Bak, Fmoc-Rink amide MBHA resin was extended using the general peptide synthesis procedure to provide the protected resin-bound peptide (1.020 g). The Mtt group was removed, labeled with 6-FAM-NHS and cleaved and deprotected as described hereinabove to provide the crude product as an orange solid (0.37 g). This product was purified by RP-HPLC. Fractions across the main peak were tested by analytical RP-HPLC, and the pure fractions were isolated and lyophilized, with the major peak providing the title compound (0.0802 g) as a yellow solid; MALDI-MS m/z=2137.1 [(M+H)⁺].

### 3.1.9. Alternative Synthesis of Peptide Probe F-Bak

In an alternative method, the protected peptide was assembled on 0.25 mmol Fmoc-Rink amide MBHA resin (Novabiochem) on an Applied Biosystems 433A automated peptide synthesizer running Fastmoc™ coupling cycles using pre-loaded 1 mmol amino acid cartridges, except for the fluorescein (6-FAM)-labeled lysine, where 1 mmol Fmoc-Lys(4-methyltrityl) was weighed into the cartridge. The N-terminal acetyl group was incorporated by putting 1 mmol acetic acid in a cartridge and coupling as described hereinabove. Selective removal of the 4-methyltrityl group was accomplished with a solution of 95:4:1 DCM:TIS:TFA (v/v/v) flowed through the resin over 15 minutes, followed by quenching with a flow of dimethylformamide. Single-isomer 6-carboxyfluorescein-NHS was reacted with the lysine side-chain in 1% DIEA in DMF and confirmed complete by ninhydrin testing. The peptide was cleaved from the resin and side-chains deprotected by treating with 80:5:5:5:2.5:2.5 TFA/water/phenol/thioanisole/triisopropylsilane: 3,6-dioxa-1,8-octanedithiol (v/v/v/v/v/v), and the crude peptide was recovered by precipitation with diethyl ether. The crude peptide was purified by reverse-phase high-performance liquid chromatography, and its purity and identity were confirmed by analytical reverse-phase high-performance liquid chromatography and matrix-assisted laser-desorption mass-spectrometry (m/z=2137.1 ((M+H)⁺).

### 3.2. Time Resolved-Fluorescence Resonance Energy Transfer (TR-FRET) Assay

The ability of exemplary Bcl-xL inhibitors W2.01-W2.62 to compete with probe F-Bak for binding Bcl-xL was demonstrated using a Time Resolved Fluorescence Resonance Energy Transfer (TR-FRET) binding assay. For the assay, test compounds were serially diluted in DMSO starting at 50 μM (2× starting concentration; 10% DMSO) and 10 μL transferred into a 384-well plate. 10 μL of a protein/probe/antibody mix was then added to each well at final concentrations listed below:

| | | | |
|---|---|---|---|
| Protein: | GST-BcL-xL | 1 | nM |
| Antibody | Tb-anti-GST | 1 | nM |
| Probe: | F-Bak | 100 | nM |

The samples were then mixed on a shaker for 1 minute and incubated for an additional 2 hours at room temperature. For each assay plate, a probe/antibody and protein/antibody/probe mixture were included as a negative and a positive control, respectively. Fluorescence was measured on the Envision (Perkin Elmer) using a 340/35 nm excitation filter and 520/525 (F-Bak) and 495/510 nm (Tb-labeled anti-his antibody) emission filters. Dissociation constants (Kᵢ) were determined using Wang's equation (Wang, 1995, FEBS Lett. 360:111-114). The TR-FRET assay can be performed in the presence of varying concentrations of human serum (HS) or fetal bovine serum (FBS). Compounds were tested both without HS and in the presence of 1% HS.

### 3.2.1. Results

The results of binding assays (Kᵢ in nanomolar) are provided in Table 2, below (in Table 2, “NT” means not tested):

TABLE 2
TR-FRET BcL-xL Binding Data

| Appln | Bcl-xL Binding | Bcl-xL Binding |
|---|---|---|
| Ex. No. | Kᵢ (nM) | Kᵢ (nM, 1% HS) |
| 1.1 | <0.001 | 0.09 |
| 1.2 | <0.001 | 0.16 |
| 1.3 | 0.0011 | 0.18 |
| 1.5 | 0.019 | 0.26 |
| 1.6 | <0.001 | 0.052 |
| 1.7 | 0.0007 | 0.24 |
| 1.8 | 0.0019 | 0.19 |
| 1.9 | <0.001 | 0.047 |
| 1.10 | <0.001 | 0.027 |
| 1.11 | <0.001 | 0.17 |
| 1.12 | <0.001 | 0.08 |
| 1.13 | <0.001 | 0.2 |
| 1.14 | <0.001 | 0.18 |
| 1.15 | 0.003 | 0.39 |
| 1.16 | <0.001 | 0.17 |
| 1.17 | 0.022 | 0.3 |
| 1.18 | <0.001 | 1.38 |
| 1.19 | NT | NT |
| 1.20 | <0.001 | 0.47 |
| 1.21 | 0.004 | 0.22 |
| 1.22 | 0.006 | 7.11 |
| 1.23 | 0.006 | 0.1 |
| 1.24 | <0.001 | 0.074 |
| 1.25 | <0.001 | 0.083 |
| 1.26 | <0.001 | 0.11 |
| 1.27 | <0.001 | 0.18 |
| 1.28 | <0.001 | 0.046 |
| 1.29 | 0.009 | 0.033 |
| 1.30 | <0.001 | 0.12 |
| 1.31 | <0.001 | 0.055 |
| 1.32 | <0.001 | 0.28 |
| 1.33 | <0.001 | 0.12 |
| 1.34 | 0.00043 | 0.031 |
| 1.35 | <0.001 | 0.439 |
| 1.36 | <0.001 | 0.0635 |
| 1.37 | <0.001 | 0.294 |
| 1.38 | <0.001 | 0.203 |
| 1.39 | 0.018 | 0.174 |
| 1.40 | <0.001 | 6.28 |
| 1.41 | 0.008 | 0.81 |
| 1.42 | <0.001 | 0.133 |
| 1.43 | <0.001 | 0.86 |
| 1.44 | <0.001 | 0.282 |
| 1.45 | <0.001 | 0.134 |
| 1.46 | <0.001 | 0.00615 |
| 1.47 | 0.006 | 0.0294 |
| 1.48 | <0.001 | 0.497 |
| 1.49 | NT | NT |
| 1.50 | 0.0073 | 0.051 |
| 1.51 | <0.001 | 0.17 |
| 1.52 | <0.001 | 0.0329 |
| 1.53 | <0.001 | 0.088 |
| 1.54 | <0.001 | 0.11 |
| 1.55 | 0.039 | 0.039 |
| 1.56 | <0.001 | 0.123 |
| 1.57 | NT | NT |
| 1.58 | NT | NT |
| 1.59 | NT | NT |
| 1.60 | <0.001 | 0.049 |
| 1.61 | <0.001 | 0.097 |
| 1.62 | <0.001 | 0.2 |
| 1.63 | <0.01 | <0.01 |
| 1.64 | <0.01 | <0.01 |
| 1.65 | <0.01 | 0.099 |
| 1.66 | NT | NT |
| 1.67 | <0.01 | 0.51 |
| 1.68 | <0.01 | 0.32 |
| 1.69 | <0.01 | 4.9 |
| 1.70 | <0.01 | 0.275 |
| 1.71 | <0.01 | 0.061 |
| 1.72 | NT | NT |
| 1.73 | 0.88 | 34 |
| 1.74 | <0.01 | 0.016 |
| 1.75 | 0.016 | 0.85 |
| 1.76 | <0.01 | 1.05 |
| 1.77 | <0.01 | 0.166 |
| 1.78 | <0.01 | <0.01 |
| 1.79 | <0.01 | 3.1 |
| 1.80 | 0.053 | 0.78 |
| 1.81 | 0.011 | 0.37 |
| 1.82 | 0.023 | 0.46 |
| 1.83 | 0.031 | 0.66 |
| 1.84 | 0.069 | 1.2 |
| 1.85 | 0.016 | 0.4 |
| 1.86 | NT | NT |
| 1.87 | <0.01 | 0.55 |
| 1.88 | 0.127 | 3 |
| 1.89 | <0.01 | 0.97 |
| 1.90 | 0.13 | 4.2 |
| 1.91 | 0.064 | 2.8 |

NT = not tested

### Example 4. Exemplary Bcl-xL Inhibitors Able to Traverse Cell Membranes Inhibit Bcl-xL in Molt-4 Cell Viability Assays

The ability of Bcl-xL inhibitors that are able to traverse cell membranes to inhibit Bcl-xL can be determined in cell-based killing assays using a variety of cell lines and mouse tumor models. For example, their activity on cell viability can be assessed on a panel of cultured tumorigenic and non-tumorigenic cell lines, as well as primary mouse or human cell populations.

In one exemplary set of conditions, Molt-4 (ATCC, Manassas, VA) human acute lymphoblastic leukemia cells were plated 12,500 cells per well in 384-well tissue culture plates (Corning, Corning, NY) in a total volume of 25 μL tissue culture medium supplemented with 10% human serum (Sigma-Aldrich, St. Louis, MO) and treated with a 3-fold serial dilution of the compounds of interest from 10 μM to 0.0005 μM. Each concentration was tested in duplicate at least 3 separate times. The number of viable cells following 48 hours of compound treatment was determined using the CellTiter-Glo® Luminescent Cell Viability Assay according to the manufacturer's recommendations (Promega Corp., Madison, WI). Compounds were tested in the presence of 10% HS.

### 4.1. Results

The results of a Molt-4 cell viability assay (EC₅₀ in nanomolar) carried out in the presence of 10% HS for exemplary Bcl-xL inhibitors of Examples 1.1-1.91 (compounds W2.01-W2.91, respectively) are provided in Table 3, below.

TABLE 3
Bcl-xL, Inhibitor In Vitro Data

| | Bcl-xL, Binding | Bcl-xL Binding | Molt-4-Viability |
|---|---|---|---|
| Ex. No. | Kᵢ (nM) | Kᵢ (nM, 1% HS) | EC₅₀ (nM, 10% HS) |
| 1.1 | <0.001 | 0.09 | 26 |
| 1.2 | <0.001 | 0.16 | 259 |
| 1.3 | 0.0011 | 0.18 | 23 |
| 1.5 | 0.019 | 0.26 | 189 |
| 1.6 | <0.001 | 0.052 | 89 |
| 1.7 | 0.0007 | 0.24 | 12 |
| 1.8 | 0.0019 | 0.19 | 108 |
| 1.9 | <0.001 | 0.047 | NT |
| 1.10 | <0.001 | 0.027 | 74 |
| 1.11 | <0.001 | 0.17 | 63 |
| 1.12 | <0.001 | 0.08 | 389 |
| 1.13 | <0.001 | 0.2 | 257 |
| 1.14 | <0.001 | 0.18 | 415 |
| 1.15 | 0.003 | 0.39 | 663 |
| 1.16 | <0.001 | 0.17 | 218 |
| 1.17 | 0.022 | 0.3 | 576 |
| 1.18 | <0.001 | 1.38 | 3328 |
| 1.19 | NT | NT | NT |
| 1.20 | <0.001 | 0.47 | 774 |
| 1.21 | 0.004 | 0.22 | 420 |
| 1.22 | 0.006 | 7.11 | >10,000 |
| 1.23 | 0.006 | 0.1 | 2,720 |
| 1.24 | <0.001 | 0.074 | 390 |
| 1.25 | <0.001 | 0.083 | 430 |
| 1.26 | <0.001 | 0.11 | 2,330 |
| 1.27 | <0.001 | 0.18 | 935 |
| 1.28 | <0.001 | 0.046 | 568 |
| 1.29 | 0.009 | 0.033 | 935 |
| 1.30 | <0.001 | 0.12 | 184 |
| 1.31 | <0.001 | 0.055 | 446 |
| 1.32 | <0.001 | 0.28 | 708 |
| 1.33 | <0.001 | 0.12 | 319 |
| 1.34 | 0.00043 | 0.031 | 87.7 |
| 1.35 | <0.001 | 0.439 | 238 |
| 1.36 | <0.001 | 0.0635 | 405 |
| 1.37 | <0.001 | 0.294 | 805 |
| 1.38 | <0.001 | 0.203 | 245 |
| 1.39 | 0.018 | 0.174 | 1,590 |
| 1.40 | <0.001 | 6.28 | 6,480 |
| 1.41 | 0.008 | 0.81 | 473 |
| 1.42 | <0.001 | 0.133 | 482 |
| 1.43 | <0.001 | 0.86 | 185 |
| 1.44 | <0.001 | 0.282 | 1260 |
| 1.45 | <0.001 | 0.134 | 19 |
| 1.46 | <0.001 | 0.00615 | 68 |
| 1.47 | 0.006 | 0.0294 | 197 |
| 1.48 | <0.001 | 0.497 | 692 |
| 1.49 | NT | NT | 92 |
| 1.50 | 0.0073 | 0.051 | 2770 |
| 1.51 | <0.001 | 0.17 | 555 |
| 1.52 | <0.001 | 0.0329 | 973 |
| 1.53 | <0.001 | 0.088 | 462 |
| 1.54 | <0.001 | 0.11 | >10,000 |
| 1.55 | 0.039 | 0.039 | 73 |
| 1.56 | <0.001 | 0.123 | 196 |
| 1.57 | NT | NT | NT |
| 1.58 | NT | NT | NT |
| 1.59 | NT | NT | NT |
| 1.60 | <0.001 | 0.049 | 425 |
| 1.61 | <0.001 | 0.097 | 255 |
| 1.62 | <0.001 | 0.2 | 153 |
| 1.63 | <0.01 | <0.01 | 2.22 |
| 1.64 | <0.01 | <0.01 | 5.25 |
| 1.65 | <0.01 | 0.099 | NT |
| 1.66 | NT | NT | NT |
| 1.67 | <0.01 | 0.51 | NT |
| 1.68 | <0.01 | 0.32 | 346 |
| 1.69 | <0.01 | 4.9 | NT |
| 1.70 | <0.01 | 0.275 | 193 |
| 1.71 | <0.01 | 0.061 | 677 |
| 1.72 | NT | NT | NT |
| 1.73 | 0.88 | 34 | 3500 |
| 1.74 | <0.01 | 0.016 | 5.7 |
| 1.75 | 0.016 | 0.85 | 409 |
| 1.76 | <0.01 | 1.05 | 495 |
| 1.77 | <0.01 | 0.166 | 837 |
| 1.78 | <0.01 | <0.01 | 2.22 |
| 1.79 | <0.01 | 3.1 | 104 |
| 1.80 | 0.053 | 0.78 | 355 |
| 1.81 | 0.011 | 0.37 | 252 |
| 1.82 | 0.023 | 0.46 | 373 |
| 1.83 | 0.031 | 0.66 | 178 |
| 1.84 | 0.069 | 1.2 | 259 |
| 1.85 | 0.016 | 0.4 | 78 |
| 1.86 | NT | NT | NT |
| 1.87 | <0.01 | 0.55 | 378 |
| 1.88 | 0.127 | 3 | 96 |
| 1.89 | <0.01 | 0.97 | NT |
| 1.90 | 0.13 | 4.2 | NT |
| 1.91 | 0.064 | 2.8 | NT |

NT = not tested

### Example 5. Exemplary Bcl-xL Inhibitors Having Low Cell Permeability Inhibit Bcl-xL in Molt-4 Cell Viability Assays with Permeabilized Cells

The ability of Bcl-xL inhibitors have low cell permeability to inhibit Bcl-xL was demonstrated in a Molt4 cell viability assay with permeabilized cells.

The permeabilization of the mitochondrial outer membrane to release proteins from the intermembrane space into the cytosol is a pivotal event in the process of apoptosis. Specifically, the release of cytochrome C triggers the formation of the apoptosome which, in turn, results in caspase activation and other events that will commit the cell to undergo programmed cell death (Goldstein et al., 2005, Cell Death and Differentiation 12:453). The process of mitochondrial outer membrane permeabilization (MOMP) is controlled by the Bcl-2 family members. It is promoted by the multidomain pro-apoptotic proteins Bax and Bak which, upon activation, oligomerize on the outer mitochondrial membrane and form pores to ultimately release cytochrome C. This action is antagonized by the antiapoptotic members, including Bcl-2 and Bcl-xL. Compounds that are capable of inhibiting Bcl-2 or Bcl-xL, for example, in cells that depend upon these proteins for survival, can cause activation of Bax and or Bak, MOMP, release of cytochrome C and the mentioned downstream steps in the apoptotic process. This process of cytochrome C release can be measured in cells via Western blot of both mitochondrial and cytosolic fractions, and can be used a proxy measurement of apoptosis in cells.

In cells that rely upon Bcl-xL for survival, Bcl-xL inhibitors that are able to permeate cells can enter cells and cause release of cytochrome C if they sufficiently inhibit Bcl-xL. Compounds that are not cell-permeable or exhibit low cell-permeability are not expected to cause release of cytochrome C, or are expected to require higher concentrations to cause release of cytochrome C.

As a means of detecting the ability of a Bcl-xL inhibitor with low cell-permeability to cause release of cytochrome C, the cells can be treated with an agent that causes selective pore formation in the plasma membrane, but not the mitochondrial membrane. Specifically, the cholesterol/phospholipid ratio is much higher in the plasma membrane than the mitochondrial membrane. As a result, short incubation with low concentrations of the cholesterol-directed detergent digitonin selectively permeabilizes the plasma membrane without significantly affecting the mitochondrial membrane. This agent forms insoluble complexes with cholesterol leading to the segregation of cholesterol from its normal phospholipid binding sites. This action, in turn, leads to the formation of holes about 40-50 Å wide in the lipid bilayer. Once the plasma membrane is permeabilized, cytosolic components able to pass over digitonin-formed holes can be washed out, including the cytochrome C that was released from mitochondria to cytosol in the apoptotic cells ((Campos, 2006, Cytometry A 69(6):515-523)).

To determine if Bcl-xL inhibitors were inducing cell death through apoptosis, cytochrome C release was measured in Bcl-xL dependent Molt-4 cells following treatment. Specifically, 1×10⁶ cells were treated for 4 h (37° C., 5% CO₂) with the test compounds in half-log dilutions starting at 3.0 μM and ending at 0.01 μM. Cells were then processed as described in Chen et al., 2011, Mol. Cancer Ther. 10:2340-2350 (the Bcl-2/Bcl-X(L)/Bcl-w inhibitor, navitoclax, enhances the activity of chemotherapeutic agents in vitro and in vivo.).

In addition to cytochrome c release, mitochondria undergoing apoptosis frequently lose their transmembrane mitochondrial membrane potential (Bouchier-Hayes et al., 2008, Methods 44(3): 222-228). JC-1 is a cationic carbocyanine dye that accumulates in mitochondria and fluoresces red when mitochondria are healthy and is lost when the mitochondrial membrane is compromised (percentage depolarization; Smiley et al., 1991, Proc. Natl. Acad. Sci. USA, 88: 3671-3675; Reers et al., 1991: Biochemistry, 30: 4480-4486). This loss in signal can be detected in permeabilized cells using a fluorimeter (excitation 545 nm and emission of 590 mu) and is therefore fully quantitative, enhancing both reproducibility and throughput. Specifically, digitonin permeabilized molt-4 cells (75,000/well) were treated with Bcl-xL inhibitors (1000 nM-0.001 nM) for up to 180 minutes at 32° C., with fluorescence determined every 10 minutes. At the maximal signal, the percentage depolarization can be determined for each time concentration of Bcl-xL inhibitors according to the formula:

% Depolarization=1−[(Sample−FCCP)/(DMSO−FCCP)]

DMSO and FCCP (10 μM) are used as negative and positive controls respectively (Ryan & Letai, 2013, Methods 61(2): 156-164). EC₅₀ values were subsequently determined from the resulting concentration-response curves. Good alignment was observed when the ability of Bcl-xL inhibitors to induce cytochrome c release was compared to loss in JC-1 fluorescence in permeabilized Molt4 cells.

### 5.1. Results

The results of the Molt-4 cell viability assay with permeabilized cells for exemplary compounds are provided in Table 4, below. Also provided for comparison are Bcl-xL binding data and the results of Molt-4 cell viability assays carried out in non-permeabilized cells. In Table 4, the following convention is used to report EC₅₀'s: “+++” corresponds to an EC₅₀<˜500 pM, “++” corresponds to an EC₅₀ between ˜500 pM and ˜1 nM and “+” corresponds to an EC₅₀ between ˜1 nM and ˜5 nM.

TABLE 4
Bcl-xL Inhibitors with Low Cell Permeability Functionally Inhibit
Bcl-xL to Release Cytochrome C and Induce Apoptosis in
Selectively Permeabilized Cells

| | | | Molt-4 |
|---|---|---|---|
| Appln | Bcl-xL Binding | Molt-4 Viability | Permeabilized |
| Ex. No. | Kᵢ (nM) | EC₅₀ (nM) | Cyt C Release |
| 1.2 | <0.001 | 259 | +++ |
| 1.5 | 0.019 | 189 | ++ |
| 1.6 | <0.001 | 89 | +++ |
| 1.10 | <0.001 | 74 | +++ |
| 1.11 | <0.001 | 63 | +++ |
| 1.12 | <0.001 | 389 | +++ |
| 1.13 | <0.001 | 257 | +++ |
| 1.14 | <0.001 | 415 | +++ |
| 1.15 | 0.003 | 663 | + |
| 1.18 | <0.001 | 3,328 | + |
| 1.20 | <0.001 | 774 | +++ |
| 1.22 | 0.006 | >10,000 | +++ |
| 1.23 | 0.006 | 2,720 | +++ |
| 1.24 | <0.001 | 390 | +++ |
| 1.25 | <0.001 | 430 | +++ |
| 1.26 | <0.001 | 2,330 | +++ |
| 1.29 | 0.009 | 935 | +++ |
| 1.32 | <0.001 | 708 | +++ |
| 1.38 | <0.001 | 245 | +++ |
| 1.39 | 0.018 | 1,590 | +++ |
| 1.41 | 0.008 | 473 | +++ |
| 1.44 | <0.001 | 1,260 | +++ |
| 1.46 | <0.001 | 68 | +++ |
| 1.47 | 0.006 | 197 | +++ |
| 1.18 | <0.001 | 692 | +++ |
| 1.49 | NT | 92 | +++ |

As can be seen from Table 4, exemplary Bcl-xL inhibitors that have low-cell permeability and that do not exhibit significant inhibitory activity in assays with non-permeabilized cells cause cytochrome C release, an on-target functional response en route to apoptosis, at sub-nanomolar concentrations in selectively permeabilized cells. In addition, percent depolarization assay results (EC₅₀ in nanomolar) in the JC-1 assay in permeabilized Molt-4 cells for representative Examples 1.23, 1.41, 1.45, 1.69, 1.76, 1.77, 1.79, 1.85, 1.87 and 1.88 are 0.24 nM, 0.56 nM, 0.13 nM, 0.14 nM, 0.26 nM, 0.099 nM, 0.14 nM, 0.07 nM, 0.06 nM and 0.21 nM, respectively. Thus, the inhibitors are expected to be functionally potent when trafficked into the cell by methods such as, but not limited to, antibody-mediated endocytosis.

### Example 6. DAR and Aggregation of Exemplary ADCs

The DAR and percentage aggregation of exemplary ADCs synthesized as described in Example 3, above, were determined by LC-MS and size exclusion chromatography (SEC), respectively.

### 6.1. LC-MS General Methodology

LC-MS analysis was performed using an Agilent 1100 HPLC system interfaced to an Agilent LC/MSD TOF 6220 ESI mass spectrometer. The ADC was reduced with 5 mM (final concentration) Bond-Breaker® TCEP solution (Thermo Scientific, Rockford, IL), loaded onto a Protein Microtrap (Michrom Bioresources, Auburn, CA) desalting cartridge, and eluted with a gradient of 10% B to 75% B in 0.2 minutes at ambient temperature. Mobile phase A was H₂O with 0.1% formic acid (FA), mobile phase B was acetonitrile with 0.1% FA, and the flow rate was 0.2 ml/min. Electrospray-ionization time-of-flight mass spectra of the co-eluting light and heavy chains were acquired using Agilent MassHunter™ acquisition software. The extracted intensity vs. m/z spectrum was deconvoluted using the Maximum Entropy feature of MassHunter software to determine the mass of each reduced antibody fragment. DAR was calculated from the deconvoluted spectrum by summing intensities of the naked and modified peaks for the light chain and heavy chain, normalized by multiplying intensity by the number of drugs attached. The summed, normalized intensities were divided by the sum of the intensities, and the summing results for two light chains and two heavy chains produced a final average DAR value for the full ADC.

### 6.2. Size Exclusion Chromatography General Methodology

Size exclusion chromatography was performed using a Shodex KW802.5 column in 0.2M potassium phosphate pH 6.2 with 0.25 mM potassium chloride and 15% IPA at a flow rate of 0.75 ml/min. The peak area absorbance at 280 nm was determined for each of the high molecular weight and monomeric eluents by integration of the area under the curve. The % aggregate fraction of the conjugate sample was determined by dividing the peak area absorbance at 280 nM for the high molecular weight eluent by the sum of the peak area absorbances at 280 nM of the high molecular weight and monomeric eluents multiplied by 100%.

### 6.3. Results

The average DAR values determined by the above LC-MS method and the % aggregate fraction for the exemplary ADCs are reported in Table 5:

TABLE 5
ADC Analytical Characterization

| Appln | | % Agg | DAR |
|---|---|---|---|
| Ex. No. | ADC Code | (by SEC) | (by MS) |
| 3.1 | AB033-CZ | 3 | 4.2 |
| 3.2 | AB033-DH | 3.2 | 4.5 |
| 3.4 | AB033-EP | 4.8 | 4 |
| 3.5 | AB033-EF | 24 | 2.9 |
| 3.6 | AB033-EG | 8.1 | 4 |
| 3.7 | AB033-EH | 4.8 | 3.5 |
| 3.8 | AB033-ER | 30.2 | 3.6 |
| 3.9 | AB033-ES | 36.2 | 3.2 |
| 3.10 | AB033-EQ | 2.7 | 3.9 |
| 3.11 | AB033-EU | 3.3 | 3.4 |
| 3.12 | AB033-EV | 3.2 | 3.6 |
| 3.13 | AB033-EW | 5.8 | 1.7 |
| 3.14 | AB033-EX | 7.2 | 2.9 |
| 3.15 | AB033-EY | 5.6 | 3 |
| 3.16 | AB033-EZ | 5.5 | 3.2 |
| 3.17 | AB033-FD | 3.4 | 3.2 |
| 3.18 | AB033-FS | 15.6 | 2.9 |
| 3.19 | AB033-FI | 1.9 | 3.1 |
| 3.20 | AB033-FV | 1.8 | 3.09 |
| 3.21 | AB033-GC | 3.5 | 3.82 |
| 3.22 | AB033-GB | 3.0 | 3.85 |
| 3.23 | AB033-FW | 1.1 | 3.20 |
| 3.24 | AB033-GD | 3.5 | 3.26 |
| 3.25 | AB033-GK | 4.2 | 3.31 |
| 3.26 | AB033-GJ | 7.0 | 3.66 |
| 3.27 | AB033-GW | 5.1 | 3.62 |
| 3.28 | AB033-HF | 25 | 3.69 |
| 3.29 | AB033-HG | 28 | 1.93 |
| 3.30 | AB033-HP | 9.3 | 3.42 |
| 3.31 | AB033-HR | 29 | 3.70 |
| 3.32 | AB033-HU | 15 | 2.66 |
| 3.33 | AB033-HT | 27 | 3.47 |
| 3.34 | AB033-HV | 7.5 | 3.8 |
| 3.35 | AB033-HZ | 27.3 | 2.3 |
| 3.36 | AB033-IA | 1.4 | 3.7 |
| 3.37 | AB033-IF | 4.6 | 2.7 |
| 3.38 | AB033-IG | 17.6 | 2.3 |
| 3.39 | AB033-IH | 24.3 | 1.6 |
| 3.40 | AB033-IJ | 1.6 | 4.59 |
| 3.41 | AB033-IK | NT | NT |
| 3.42 | AB033-IL | 1.6 | 3.61 |
| 3.43 | AB033-IM | 25.6 | 2.5 |
| 3.44 | AB033-IO | 3.0 | 2.5 |
| 3.45 | AB033-IP | 18.0 | 2.1 |
| 3.46 | AB033-IS | 46.8 | 3.2 |
| 3.47 | AB033-IU | 5.6 | 3.7 |
| 3.48 | AB033-IV | 4.9 | 3.1 |
| 3.49 | AB033-IZ | 13.4 | 2.66 |
| 3.50 | AB033-JK | 23 | 4.1 |
| 3.51 | AB033-JF | 4.0 | 3.8 |
| 3.52 | AB033-FE | 1.4 | 3.4 |
| 3.53 | AB033-GG | 0.9 | 3.10 |
| 3.54 | AB033-GM | 0.8 | 2.90 |
| 3.55 | AB033-HD | 0.9 | 3.09 |
| 3.56 | AB033-HS | 0.8 | 2.94 |
| 3.57 | AB033-HW | 1.8 | 2.9 |
| 3.58 | AB033-HX | 1.1 | 1.7 |
| 3.59 | AB033-HY | 1.9 | 0.8 |
| 3.60 | AB033-IB | 1.1 | 1.9 |
| 3.61 | AB033-IE | 0.9 | 2.3 |
| 3.62 | AB033-II | 1.4 | 2.4 |
| 3.63 | AB033-JJ | 3.7 | 3.8 |
| 3.64 | AB033-IW | 2.3 | 3.1 |
| 3.65 | AB033-IY | 1.1 | 3.3 |
| 3.66 | AB033-JA | 1.0 | 3.4 |
| 3.74 | AB033-FA | 2.1 | 3.6 |
| 3.76 | AB033-FK | 3.4 | 3.4 |
| 3.77 | AB033-FR | 3.1 | 2.5 |
| 3.78 | AB033-JE | 14.6 | 5.9 |
| 3.79 | AB033-JL | 3.1 | 4.42 |
| 3.80 | AB033-LE | 11.3 | 3.9 |
| 3.81 | AB033-LH | 5.5 | 2.9 |
| 3.82 | AB033-LJ | 3.6 | 3.9 |
| 3.83 | AB033-MA | 5.3 | 4.3 |
| 3.84 | AB033-MD | 1.6 | 3.4 |
| 3.85 | AB033-MG | 10.4 | 3.8 |
| 3.86 | AB033-MS | 5.5 | 3.9 |
| 3.87 | AB033-MR | 9.6 | 4 |
| 3.88 | AB033-MQ | 5 | 2.6 |
| 3.89 | AB033-MZ | 5.6 | 1.7 |
| 3.90 | AB033-NA | 13.4 | 2.9 |
| 3.91 | AB033-NB | 18.5 | 3.8 |
| 3.92 | AB033-NN | 1.9 | 3.1 |
| 3.93 | AB033-NO | 2.3 | 3.5 |
| 3.94 | EpCAM(ING-1)-CZ | 1.67 | 3.2 |
| 3.96 | EpCAM(ING-1)-FE | 1.30 | 2.44 |
| 3.97 | EpCAM(ING-1)-GG | 0.60 | 2.27 |
| 3.98 | EpCAM(ING-1)-GM | 0.70 | 2.25 |
| 3.99 | EpCAM(ING-1)-HD | 0.90 | 3.02 |
| 3.100 | EpCAM(ING-1)-HS | 0.50 | 2.92 |
| 3.101 | EpCAM(ING-1)-HW | 0.80 | 2.73 |
| 3.102 | EpCAM(ING-1)-HX | 0.50 | 3.09 |
| 3.103 | EpCAM(ING-1)-HY | 0.70 | 2.46 |
| 3.104 | EpCAM(ING-1)-IB | 0.50 | 3.11 |
| 3.105 | EpCAM(ING-1)-IE | 0.90 | 2.88 |
| 3.106 | EpCAM(ING-1)-IJ | 0.70 | 3.52 |
| 3.107 | EpCAM(ING-1)-IK | 1.50 | 2.53 |
| 3.108 | EpCAM(ING-1)-IL | 0.70 | 3.35 |
| 3.123 | N901-CZ | 1.73 | 3.1 |
| 3.124 | AB033-OK | 9.1 | 2.69 |
| 3.125 | AB033-OW | 4.6 | 3.01 |
| 3.126 | AB033-PC | 10.34 | 1.59 |
| 3.127 | AB033-PI | 1.7 | 1.74 |
| 3.128 | AB033-PJ | 5.41 | 3.26 |
| 3.129 | AB033-PU | 9.34 | 2.55 |
| 3.130 | AB033-PV | 3.0 | 1.72 |
| 3.131 | AB033-PW | 8.88 | 2.25 |
| 3.132 | AB033-QW | 4.5 | 0.83 |
| 3.133 | AB033-RM | 4.4 | 1.2 |
| 3.134 | AB033-RR | 53.3 | 1.9 |
| 3.135 | AB033-SI | 3.6 | 2.8 |
| 3.136 | AB033-SM | 9.9 | 3.1 |
| 3.137 | AB033-SN | 1.8 | 3.3 |
| 3.138 | AB033-SS | 1.0 | 3.0 |
| 3.139 | AB033-TA | 1.2 | 3.1 |
| 3.140 | AB033-TW | 56 | 3.3 |
| 3.141 | AB033-ST | 26.3 | 1.7 |
| 3.142 | AB033-ZL | 1.7 | 2.5 |
| 3.143 | AB033-SX | 0.5 | 3.7 |
| 3.144 | AB033-SW | 1.3 | 3.4 |
| 3.145 | AB033-TV | 0.5 | 3.4 |
| 3.146 | AB033-SZ | 0.7 | 3.6 |
| 3.147 | AB033-ZM | 1.1 | 4.7 |
| 3.148 | AB033-SV | 2.9 | 2.8 |
| 3.149 | AB033-SY | 0.8 | 3.5 |
| 3.150 | AB033-TK | 1.1 | 5.2 |
| 3.151 | AB033-TR | 0.3 | 3.0 |
| 3.152 | AB033-TY | 0.3 | 3.5 |
| 3.153 | AB033-TX | <0.1 | 3.4 |
| 3.154 | AB033-TZ | 5.9 | 8.0 |
| 3.155 | AB033-UA | 0.6 | 3.8 |
| 3.156 | AB033-UJ | 0.6 | 3.5 |
| 3.157 | AB033-UK | <0.1 | 3.2 |
| 3.158 | AB033-UU | 1.8 | 4.6 |
| 3.159 | AB033-UV | 1.4 | 4.5 |
| 3.160 | AB033-UZ | 3.5 | 3.8 |
| 3.161 | AB033-VB | 0.5 | 3.7 |
| 3.162 | AB033-VC | 4.5 | 4.2 |
| 3.163 | AB033-VS | 0.9 | 3.6 |
| 3.164 | AB033-VT | 1.3 | 3.5 |
| 3.165 | AB033-VY | 2.6 | 3.9 |
| 3.166 | AB033-WI | 2.3 | 3.6 |
| 3.167 | AB033-WK | 2.4 | 3.8 |
| 3.168 | AB033-WP | 1.1 | 3.4 |
| 3.169 | AB033-XD | 1.1 | 2.7 |
| 3.170 | AB033-XK | 0.6 | 2.5 |
| 3.171 | AB033-XL | 1.5 | 2.9 |
| 3.172 | AB033-YJ | 4.4 | 1.5 |
| 3.173 | AB033-YQ | 0.6 | 4.8 |
| 3.174 | AB033-YR | 0.6 | 4.9 |
| 3.175 | AB033-YS | 2 | 4.5 |
| 3.176 | AB033-YY | 1.1 | 3.9 |
| 3.177 | AB033-YT | 0.9 | 4.2 |
| 3.178 | AB033-YU | 1.5 | 4.6 |
| 3.179 | AB033-YV | 1.6 | 4.3 |
| 3.180 | AB033-YW | 1.2 | 4.8 |
| 3.181 | AB033-ZB | 1.4 | 4.6 |
| 3.182 | AB033-ZC | 0.8 | 4.5 |
| 3.183 | AB033-ZJ | 0.7 | 4.4 |
| 3.184 | AB033-ZE | 0.7 | 3.8 |
| 3.185 | AB033-ZW | 1.3 | 1.3 |
| 3.186 | AB033-ZX | 1.5 | 4.3 |
| 3.187 | AB033-AAA | 1.5 | 5.9 |
| 3.188 | AB033-AAD | 2.3 | 5.8 |
| 3.189 | AB033-AAE | 3 | 6.3 |
| 3.190 | AB033-AAF | 1.1 | 6 |
| 3.191 | AB033-JL (E2) | 0.54 | 2 |
| 3.192 | AB033-JL (E4) | 2.2 | 4 |
| 3.193 | AB033-TX (E2) | 1.4 | 2 |
| 3.194 | AB033-TX (E4) | 6.1 | 4 |

NT = not tested

### Example 7. EGFR-Targeted ADCs Inhibit the Growth of Cancer Cells In Vitro

Certain exemplary ADCs comprising antibody AB033 was evaluated. Antibody AB033 targets human EGFR. The variable heavy and light chain sequences of antibody AB033 are described in WO 2009/134776 (see page 120). The ability of antibody AB033 to inhibit the growth of cancer cells was demonstrated with mcl-1′ mouse embryonic fibroblast (MEF) cells. Mcl-1^{-/-} MEFs are dependent upon Bcl-xL for survival (Lessene et al., 2013, Nature Chemical Biology 9:390-397). To evaluate the efficacy of exemplary AB033-targeted Bcl-xL-ADCs, human EGFR was over-expressed in mcl-1^{-/-} MEFs.

### 7.1. Method

Retroviral supernatants were produced through transfection of the GP2-293 packaging cell line (Clontech) with the retroviral construct pLVC-IRES-Hygro (Clontech) containing huEGFR sequence or the empty vector utilizing FuGENE 6 transfection reagent (Roche Molecular Biochemicals, Mannheim. Germany). After 48 hours of culture, virus-containing supernatant was harvested and applied to mcl-1^{-/-} MEFs in 75 cm² culture flasks (0.5×10⁶ per flask) for a further 48 hrs in the presence of polybrene (8 μg/ml; Sigma). Mcl-1^{-/-} MEFs were washed and selected after 3 days with 250 μg/ml hygromycin B (Invitrogen) in the full complement of media. The expression of huEGFR was confirmed by flow cytometry and compared to the parental cell line or those transfected with the empty vector.

Mcl-1^{-/-} MEFs expressing huEGFR or the pLVX empty vector (Vct Ctrl) were treated with EGFR-targeted Bcl-xL-ADCs, AB033 alone or MSL109-targeted Bcl-xL-ADCs for 96 hours in DMEM containing 10% FBS. For the assay, the cells were plated at 250 cells per well in 384-well tissue culture plates (Corning, Corning, NY) in a total volume of 25 μL of assay media (DMEM and 10% HI FBS). The plated cells were treated with a 4-fold serial dilution of the Antibody Drug Conjugates of interest from 1 pM to 1 pM dispensed by an Echo 550 Acoustic Liquid Handler (Labcyte). Each concentration was tested in twelve replicates for the mcl-1^{-/-} MEF huEGFR cell line and in six replicates for the mcl-1^{-/-} MEF vector cell line. The fraction of viable cells following 96 hours of Antibody Drug Conjugate treatment at 37° C. and 5% CO₂ was determined using the CellTiter-Glo® Luminescent Cell Viability Assay according to the manufacturer's recommendations (Promega Corp., Madison, WI). The plates were read in a Perkin Elmer Envision using a Luminescence protocol with 0.5 sec integration time. The replicate values for each dilution point were averaged and the EC₅₀ values for the Antibody Drug Conjugates were generated by fitting the data with GraphPad Prism 5 (GraphPad Software, Inc.) to a sigmoidal curve model using linear regression, Y=((Bottom−Top)/(1+((x/K)ⁿ)))+Top, where Y is the measured response, x is the compound concentration, n is the Hill Slope and K is the EC₅₀ and Bottom and Top are the lower and higher asymptotes respectively. Visual inspection of curves was used to verify curve fit results. Mcl-1^{-/-} MEFs were obtained from David C. S. Huang of the Walter and Eliza Hall Institute of Medical Research.

### 7.2. Results

Cell viability assay results (EC₅₀ in nanomolar) for representative Examples are provided below in Table 6, below:

| | | huEGFR⁺ mcl-1^{-/} |
|---|---|---|
| Appln | | MEF⁻ |
| Ex. No. | ADC Code | EC₅₀ (nM) |
| 3.1 | AB033-CZ | 0.21 |
| 3.2 | AB033-DH | NT |
| 3.4 | AB033-EP | 0.88 |
| 3.5 | AB033-EF | 3.7 |
| 3.6 | AB033-EG | 2.6 |
| 3.7 | AB033-EH | 0.86 |
| 3.8 | AB033-ER | >67 |
| 3.9 | AB033-ES | >67 |
| 3.10 | AB033-EQ | 0.85 |
| 3.11 | AB033-EU | 0.93 |
| 3.12 | AB033-EV | 0.91 |
| 3.13 | AB033-EW | 0.87 |
| 3.14 | AB033-EX | 3.76 |
| 3.15 | AB033-EY | 4.49 |
| 3.16 | AB033-EZ | 2.18 |
| 3.17 | AB033-FD | 0.38 |
| 3.18 | AB033-FS | 28.2 |
| 3.19 | AB033-FI | 0.89 |
| 3.20 | AB033-FV | 4.41 |
| 3.21 | AB033-GC | 0.51 |
| 3.22 | AB033-GB | 0.46 |
| 3.73 | AB033-FW | 147.6 |
| 3.24 | AB033-GD | 0.94 |
| 3.25 | AB033-GK | 4.04 |
| 3.26 | AB033-GJ | 2.96 |
| 3.27 | AB033-GW | 0.25 |
| 3.28 | AB033-HF | 3.92 |
| 3.29 | AB033-HG | 3.94 |
| 3.30 | AB033-HP | 2.24 |
| 3.31 | AB033-HR | 20.75 |
| 3.32 | AB033-HU | 61.1 |
| 3.33 | AB033-HT | 55.28 |
| 3.34 | AB033-HV | 5.1 |
| 3.35 | AB033-HZ | 9.3 |
| 3.36 | AB033-IA | 132 |
| 3.37 | AB033-1F | 3.2 |
| 3.38 | AB033-IG | 1.92 |
| 3.39 | AB033-IH | 1.4 |
| 3.40 | AB033-IJ | 3.2 |
| 3.41 | AB033-IK | NT |
| 3.42 | AB033-IL | 47.9 |
| 3.43 | AB033-IM | 22.7 |
| 3.45 | AB033-IP | 6.5 |
| 3.46 | AB033-IS | 102 |
| 3.47 | AB033-IU | 809 |
| 3.48 | AB033-IV | 38.7 |
| 3.49 | AB033-IZ | 1.7 |
| 3.50 | AB033-JK | 161.3 |
| 3.51 | AB033-JF | 159.3 |
| 3.52 | AB033-FE | 0.94 |
| 3.53 | AB033-GG | 19.21 |
| 3.54 | AB033-GM | 459 |
| 3.55 | AB033-HD | >67 |
| 3.56 | AB033-HS | 3.56 |
| 3.57 | AB033-HW | >1000 |
| 3.58 | AB033-HX | 97.7 |
| 3.59 | AB033-HY | 265 |
| 3.60 | A9033-IB | >1000 |
| 3.61 | AB033-IE | 164 |
| 3.62 | AB033-II | 5 |
| 3.63 | AB033-JJ | 0.58 |
| 3.64 | AB033-IW | 226 |
| 3.65 | AB033-IY | >1000 |
| 3.66 | AB033-JA | 6.38 |
| 3.75 | AB033-FJ | >67 |
| 3.76 | AB033-FK | >67 |
| 3.77 | AB033-FR | 28.1 |
| 3.78 | AB033-JE | 20 |
| 3.79 | AB033-JL | 5.0 |
| 3.80 | AB033-LE | 10.26 |
| 3.81 | AB033-LH | 0.706 |
| 3.82 | AB033-LJ | 0.42 |
| 3.83 | AB033-MA | 29.25 |
| 3.84 | AB033-MD | 35.78 |
| 3.85 | AB033-MG | 56.92 |
| 3.86 | AB033-MS | 389.6 |
| 3.87 | AB033-MR | 220.5 |
| 3.88 | AB033-MQ | 18.65 |
| 3.89 | AB033-MZ | 1.384 |
| 3.90 | AB033-NA | 316.0 |
| 3.91 | AB033-NB | 205.7 |
| 3.92 | AB033-NN | 3.21 |
| 3.93 | AB033-NO | 46.92 |
| 3.124 | AB033-OK | 53 |
| 3.125 | AB033-OW | <1 |
| 3.126 | AB033-PC | 36 |
| 3.127 | AB033-PI | 3 |
| 3.128 | AB033-PJ | 47 |
| 3.129 | AB033-PU | 173 |
| 3.130 | AB033-PV | 616 |
| 3.131 | AB033-PW | 37 |
| 3.132 | AB033-QW | >1000 |
| 3.133 | AB033-RM | >1000 |
| 3.134 | AB033-RR | 29 |
| 3.135 | AB033-SJ | 27 |
| 3.136 | AB033-SM | 210 |
| 3.137 | AB033-SN | 2 |
| 3.138 | AB033-SS | 9 |
| 3.139 | AB033-TA | 6 |
| 3.140 | AB033-TW | 517 |
| 3.141 | AB033-ST | 224 |
| 3.142 | AB033-ZL | NT |
| 3.143 | AB033-SX | 1.2 |
| 3.144 | AB033-SW | 2.2 |
| 3.145 | AB033-TV | 2.2 |
| 3.146 | AB033-SZ | 2.8 |
| 3.147 | AB033-ZM | 13 |
| 3.148 | AB033-SV | 2 |
| 3.149 | AB033-SY | 2.6 |
| 3.150 | AB033-TK | 40 |
| 3.151 | AB033-TR | 15 |
| 3.152 | AB033-TY | 3.7 |
| 3.153 | AB033-TX | 1.9 |
| 3.154 | AB033-TZ | 8.7 |
| 3.155 | AB033-UA | 7.2 |
| 3.156 | AB033-UJ | 2.6 |
| 3.157 | AB033-UK | 2.8 |
| 3.158 | AB033-UU | <1 |
| 3.159 | AB033-UV | 62 |
| 3.160 | AB033-UZ | >1000 |
| 3.161 | AB033-VB | 2.6 |
| 3.162 | AB033-VC | 881 |
| 3.163 | AB033-VS | 2.4 |
| 3.164 | AB033-VT | 2.9 |
| 3.165 | AB033-VY | >1000 |
| 3.166 | AB033-WI | >1000 |
| 3.167 | AB033-WK | >1000 |
| 3.168 | AB033-WP | 2.1 |
| 3.169 | AB033-XD | 5.8 |
| 3.170 | AB033-XK | 2.7 |
| 3.171 | AB033-XL | 2.7 |
| 3.172 | AB033-YJ | 7.7 |
| 3.173 | AB033-YQ | <1 |
| 3.174 | AB033-YR | <1 |
| 3.175 | AB033-YS | 578 |
| 3.176 | AB033-YY | 555 |
| 3.177 | AB033-YT | 1.1 |
| 3.178 | AB033-YU | 4.5 |
| 3.179 | AB033-YV | 4.3 |
| 3.180 | AB033-YW | 2.2 |
| 3.181 | AB033-ZB | 3.3 |
| 3.182 | AB033-ZC | 2 |
| 3.183 | AB033-ZJ | <1 |
| 3.184 | AB033-ZE | >1 |
| 3.185 | AB033-ZW | 12 |
| 3.186 | AB033-ZX | <1 |
| 3.187 | AB033-AAA | 9.8 |
| 3.188 | AB033-AAD | 4.7 |
| 3.189 | AB033-AAE | 131 |
| 3.190 | AB033-AAF | 171 |
| 3.191 | AB033-JL (E2) | 17 |
| 3.192 | AB033-JL (E4) | 2 |
| 3.193 | AB033-TX (E2) | 32 |
| 3.194 | AB033-TX (E4) | 4.7 |

NT = not tested

Cell viability assay results (EC₅₀ in nanomolar) for representative Examples 3.1, 3.79, 3.143, 3.145, 3.153, 3.175, 3.183, 3.186 and 3.190 against the Mcl-1^{-/-} MEF vector cell line are 99 nM, 103 nM, 96 nM, 239 nM, >1,000 nM, >250 nM, 148 nM and 662 nM, respectively.

### EpCAM and NCAM1—Targeted ADCs Inhibit the Growth of Cancer Cells In Vitro

The ability of certain exemplary ADCs comprising an antibody that targets human cell adhesion molecule (EpCAM) to inhibit Bcl-xL and induce apoptosis was demonstrated with NCC38 cells, a human breast cancer cell line expressing endogenous EpCAM protein. ADCs comprising the monoclonal antibody ING-1 (see Studnicka et al., 1994, Protein Engineering, 7:805-814 and Ammons et al., 2003, Neoplasia 5:146-154), which targets EpCAM, were evaluated in the assay.

Cytotoxicity of certain exemplary ADCs targeted to human neural cell adhesion molecule NCAM-1 was demonstrated in NCI-H146 cells, a human small cell lung cancer line that expresses endogenous NCAM-1. The ADCs evaluated comprised the monoclonal NCAM-1 antibody referred to as N901. See Roguska et al., 1994, Proc Natl Acad Sci USA 91:969-973).

### 7.3. Method

For the assay, both HCC38 and NCI-H146 cell lines were cultured in RPMI 1640 media (Invitrogen, #11995) containing 10% FBS. Prior to the assay, cells were resuspended to 4×10⁴ cells/ml in culture media and then added to the 96-well tissue culture plates at 75 μL cells/well for a final concentration of 3,000 cells/well. The assay plates were then incubated at 37° C. with 5% CO₂ for overnight. On the following day, N901, EpCAM(ING-1) or negative control (MSL109, an antibody targeting CMV) ADCs were serially diluted in culture media and were added to the assay plates at 25 μL/well. The assay plates were then incubated at 37° C. with 5% CO₂ for 72 hours. Cell viability was measured by the CellTiter-Glo® Luminescent Cell Viability Assay Kit (Promega, #G7573).

### 7.4. Results

Data was analyzed using GraphPad Prism software. The IC₅₀ values (the concentration of ADC to achieve 50% of the maximum growth inhibition of the cells) are reported in Tables 7 and 8.

As shown in Table 7, EpCAM-targeted ADCs potently killed HCC38 breast cancer cells (IC₅₀≤0.4 nM) while the negative control ADCs MSL109-CZ showed weak activity. As seen in Table 8, NCAM1-targeted ADCs also showed specific activity toward NCI-H146 small cell lung cancer cells (IC₅₀˜20 nM) whereas the negative control MSL109-targeted ADC showed weak activity.

TABLE 7
EpCAM ADCs Inhibit the Growth of HCC38
Breast Cancer Cells

| Appln | | HCC38 Cells |
|---|---|---|
| Ex. No. | ADC Code | IC₅₀ (nM) |
| 3.94 | Epcam(ING-1)-CZ | 0.10 |
| | MSL109-CZ | 41.29 |

As seen in Table 8, NCAM1-targeted ADCs also showed specific activity toward NCI-H146 small cell lung cancer cells (IC₅₀˜20 nM) whereas the negative control MSL109-targeted ADC showed weak activity.

TABLE 8
NCAM1 ADCs Inhibit the Growth of NCI-H146
Small Cell Lung Cancer Cells

| Appln | | NCI-H146 Cells |
|---|---|---|
| Ex. No. | ADC Code | IC₅₀ (nM) |
| 3.123 | N901-CZ | 19.57 |
| | M5L109-CZ | 329.85 |

### Example 8. Exemplary EGFR-Targeted Bcl-xL Inhibitory ADCs (Bcl-xLi ADCs) Inhibit the Growth of Tumors In Vivo Following Administration of a Single Dose

The ability of certain exemplary EGFR-targeted ADCs to inhibit the growth of tumor cells in vivo in mice was demonstrated in a xenograft model with tumors derived from NCI-H1650 cells, a human non small cell lung cancer (NSCLC) cell line.

### 8.1. Method

The NSCLC cell line NCI-H1650 was purchased from the American Type Culture Collection (ATCC, Manassas, VA). The cells were cultured as monolayers in RPMI 1640 culture medium (Invitrogen, Carlsbad, CA) that was supplemented with Fetal Bovine Serum (FBS, Hyclone, Logan, UT). Five million viable cells NCI-H1650 cells were inoculated subcutaneously into the right flank of immune deficient female SCID/bg mice (Charles River Laboratories, Wilmington, MA). The injection volume was 0.2 ml and composed of a 1:1 mixture of S-MEM and Matrigel (BD, Franklin Lakes, NJ). Tumors were size matched at approximately 200 mm³. Antibodies and conjugates were formulated in phosphate buffered saline (PBS) and injected intraperitoneally. Injection volume did not exceed 400 μl. Therapy began within 24 hours after size matching of the tumors. Mice weighed approximately 25 g at the onset of therapy. Tumor volume was estimated two to three times weekly. Measurements of the length (L) and width (W) of the tumor were taken via electronic caliper and the volume was calculated according to the following equation: V=L×W²/2. Mice were euthanized when tumor volume reached 3,000 mm³ or skin ulcerations occurred. Eight to ten mice were housed per cage. Food and water were available ad libitum. Mice were acclimated to the animal facilities for a period of at least one week prior to commencement of experiments. Animals were tested in the light phase of a 12-hour light: 12-hour dark schedule (lights on at 06:00 hours). All experiments were conducted in compliance with AbbVie's Institutional Animal Care and Use Committee and the National Institutes of Health Guide for Care and Use of Laboratory Animals guidelines in a facility accredited by the Association for the Assessment and Accreditation of Laboratory Animal Care.

The EGFR-targeted ADCs 3.1, 3.2, 3.12, 3.20, 3.23, 3.21, 3.53, 3.25, 3.54, 3.55, 3.31, 3.32, 3.57, 3.59, 3.36, 3.79, 3.30, 3.78, 3.24, 3.48, 3.40, 3.62, 3.47, 3.79, 3.143, 3.144, 3.145. 3.146, 3.149, 3.151, 3.152, 3.153, 3.154, 3.155, 3.156, 3.157, 3.160, 3.161, 3.162, 3.163, 3.164, 3.165, 3.166, 3.167, 3.193, 3.194, 3.195 and 3.196 were prepared according to procedures in Example 3 (Synthesis of exemplary ADCs), Table 1. A conjugate (MSL109-H) of synthon H (see Example 2.99) and the CMV targeting antibody MSL109 was used as a passive targeting control. This conjugate is hereafter also referred to as ‘non-targeting’ ADC because the carrier antibody does not recognize a tumor associated antigen. MSL109 is described in Drobyski et al., 1991, Transplantation 51:1190-1196 and U.S. Pat. No. 5,750,106. An antibody that targets tetanus toxoid (antibody AB095) was used as a control for the effect of administering IgG see Larrick et al., 1992, Immunological Reviews 69-85. The efficacy of inhibition of H1650 xenograft growth with EGFR-targeted ADCs is illustrated by Table 9, 10, 11, and 12, below. The tumor growth inhibition by EGFR-targeting control antibody and ‘non-targeting’ ADCs is described in Table 13. Treatment was initiated at 11 days at the earliest (Table 9) or at latest 15 days (Table 12) post inoculation of tumor cells. The tumor size at onset of treatment was between 210 mm³ and 230 mm³. All conjugates and antibodies were given intraperitoneally. The doses and regimens of treatment are specified in the tables.

### 8.2. Results

### 8.2.1. Parameters of Efficacy and Statistical Analysis

The efficacy of inhibition of H1650 xenograft growth with EGFR-targeted ADCs is illustrated in Table 9, Table 10, Table 11 and Table 12, below. In the tables, to refer to efficacy, parameters of amplitude (TGIₘₐₓ) and durability (TGD) of therapeutic response are used.

TGIₘₐₓ is the maximum tumor growth inhibition during the experiment. Tumor growth inhibition is calculated by 100*(1−Tᵥ/Cᵥ) where Tᵥ and Cᵥ are the mean tumor volumes of the treated and control groups, respectively.

TGD or tumor growth delay is the extended time of a treated tumor needed to reach a volume of 1 cm³ relative to the control group. TGD is calculated by 100*(Tₜ/Cₜ−1) where Tₜ and Cₜ are the median time periods to reach 1 cm³ of the treated and control groups, respectively.

Distribution of the response amplitude in a specific group is given by the frequency of complete responders (CR), partial responders (PR), and overall responders (OR). CR is the percentage of mice within a group with a tumor burden of 25 mm³ for at least three measurements. PR is the percentage of mice within a group with a tumor burden larger than 25 mm³ but less than one-half of the volume at onset of treatment for at least three measurements. OR is the sum of CR and PR.

The 2-tailed Student's test and Kaplan-Meier log-rank test were used to determine significance of the difference in TGIₘₐₓ and TGD, respectively.

### 8.2.2. Efficacy of EGFR-Targeting Bcl-xLi ADCs In Vivo

The cytomegalovirus (CMV)-targeting ADC MSL109-H, at a dose of 10 mg/kg, inhibited tumor growth by 20% (Table 9). This inhibition is associated with passive targeting (Boghaert et al., 2006, Int. J. Oncol., 28 (3):675-684). The efficacy achieved with passive targeting is inferior to the efficacy seen with the ADCs that use the EGFR-targeting antibody, AB033. The TGIₘₐₓ of EGFR-targeted ADC AB033-CZ at 10 mg/kg is between 93 and 99% (Table 9 and Table 10, respectively) and the TGD is between 153 (Table 11) and >507% (Table 9). The TGIₘₐₓ of other conjugates consisting of AB033 and Bcl-xL-targeting synthons was between 41% and 99% and TGD between 11% and >507% (Table 11 and Table 9, respectively). For the experiment presented in Table 9, the TGIₘₐₓ of EGFR-targeted ADCs are between 3.5-4.9 fold higher than the TGIₘₐₓ of the non-targeting ADC MSL109-H. The response to EGFR-targeting ADCs is also more durable than that of MSL109-H as shown by 6 to >72-fold increase in TGD.

In Tables 9-12, the lowest activity observed was following treatment with AB033-UJ. This conjugate inhibited tumor growth by 44% and caused a tumor growth delay of 11%. The efficacy of the EGFR-targeting BclxLi conjugates is unlikely due to the activity of the carrier antibody or to activity from passive targeting. Historical controls (Table 13) show that the minimum total amount of AB033 necessary to match the efficacy of AB033-UJ is approximately 18 mg/kg given as 6 doses of 3 mg/kg with an interval of 4 days. The non-targeting ADCs, MSL109-H and MSL 109-CZ approximated the efficacy of AB033-UJ when a total amount of 60 mg/kg was administered. Treatment with AB033, MSL 109-CZ or MSL109-H induced neither complete nor partial responses.

TABLE 9
Inhibition of H1650 xenograft tumor growth after treatment
with a single dose of EGFR-targeting Bcl-xLi ADCs

| | | | Growth | | | | |
|---|---|---|---|---|---|---|---|
| | | | Inhibition | | Response Frequency | | |
| Appln | | Dose^{[a]}/route/ | TGIₘₐₓ | TGD | CR | PR | OR |
| No. | Treatment | regimen | (%) | (%) | (%) | (%) | (%) |
| | AB095**^{, †} | 10/IP/QDx1 | 0 | 0 | 0 | 0 | 0 |
| | MSL109-H^{†} | 10/IP/QDx1 | 20 | 7* | 0 | 0 | 0 |
| 3.1 | AB033-CZ | 10/IP/QDx1 | 98* | >507* | 100 | 0 | 100 |
| 3.2 | AB033-DH | 10/IP/QDx1 | 97* | >507* | 88 | 13 | 100 |
| 3.12 | AB033-EV | 10/IP/QDx1 | 97* | 207* | 38 | 63 | 100 |
| 3.20 | AB033-FV | 10/IP/QDx1 | 98* | >507* | 75 | 25 | 100 |
| 3.23 | AB033-FW | 10/IP/QDx1 | 92* | 170* | 0 | 88 | 88 |
| 3.21 | AB033-GC | 10/IP/QDx1 | 98* | 290* | 50 | 50 | 100 |
| 3.53 | AB033-GG | 10/IP/QDx1 | 98* | 300* | 38 | 63 | 100 |
| 3.25 | AB033-GK | 10/IP/QDx1 | 96* | 460* | 75 | 25 | 100 |
| 3.54 | AB033-GM | 10/IP/QDx1 | 78* | 73* | 0 | 25 | 25 |
| 3.55 | AB033-HD | 10/IP/QDx1 | 96* | >507* | 13 | 88 | 100 |
| 3.31 | AB033-HR | 10/IP/QDx1 | 82* | 133* | 0 | 38 | 38 |
| 3.32 | AB033-HU | 10/IP/QDx1 | 69* | 50* | 0 | 0 | 0 |
| 3.57 | AB033-HW | 10/IP/QDx1 | 75* | 50* | 0 | 0 | 0 |
| 3.59 | AB033-HY | 10/IP/QDx1 | 85* | 193* | 13 | 63 | 75 |
| 3.36 | AB033-IA | 10/IP/QDx1 | 69* | 40* | 0 | 13 | 13 |

**IgG1 mAb
^{†}Non-targeting antibody
^{[a]}dose is given in mg/kg/day
*= p < 0.05 as compared to control treatment (AB095)

TABLE 10
Inhibition of H1650 xenograft tumor growth after treatment
with a single dose of EGFR-targeting Bcl-xLi ADCs

| | | | Growth | | | | |
|---|---|---|---|---|---|---|---|
| | | | Inhibition | | Response Frequency | | |
| | | Dose^{[a]}/route/ | TGIₘₐₓ | TGD | CR | PR | OR |
| Ex. No. | Treatment | regimen | (%) | (%) | (%) | (%) | (%) |
| IgG1 | AB095**^{, †} | 10/IP/QDx1 | 0 | 0 | 0 | 0 | 0 |
| mAb | | | | | | | |
| 3.1 | AB033-CZ | 10/IP/QDx1 | 99* | >500* | 100 | 0 | 100 |
| 3.79 | AB033-JL | 10/IP/QDx1 | 97* | >500* | 38 | 63 | 100 |
| 3.30 | AB033-HP | 10/IP/QDx1 | 99* | >500* | 100 | 0 | 100 |
| 3.78 | AB033-JE | 10/IP/QDx1 | 83* | 60* | 0 | 0 | 0 |
| 3.24 | AB033-GD | 10/IP/QDx1 | 98* | >500* | 88 | 13 | 100 |
| 3.48 | AB033-IV | 10/IP/QDx1 | 97* | >500* | 88 | 13 | 100 |
| 3.40 | AB033-IJ | 10/IP/QDx1 | 99* | >500* | 75 | 25 | 100 |
| 3.62 | AB033-II | 10/IP/QDx1 | 98* | >500* | 100 | 0 | 100 |
| 3.47 | AB033-IU | 10/IP/QDx1 | 53* | 23* | 0 | 0 | 0 |

**IgG1 mAb
^{†}Non-targeting antibody
^{[a]}dose is given in mg/kg/day
*= p < 0.05 as compared to control treatment (AB095)

TABLE 11
Inhibition of H1650 xenograft tumor growth after treatment
with a single dose of EGFR-targeting Bcl-xLi ADC

| | | | Growth | | | | |
|---|---|---|---|---|---|---|---|
| | | | Inhibition | | Response Frequency | | |
| | | Dose^{[a]}/route/ | TGIₘₐₓ | TGD | CR | PR | OR |
| Ex. No. | Treatment | regimen | (%) | (%) | (%) | (%) | (%) |
| | AB095**^{, †} | 10/IP/QDx1 | 0 | 0 | 0 | 0 | 0 |
| 3.1 | AB033-CZ | 10/IP/QDx1 | 93* | 153* | 13 | 88 | 100 |
| 3.79 | AB033-JL | 10/IP/QDx1 | 90* | 137* | 0 | 88 | 88 |
| 3.193 | AB033-JL (E2) | 10/IP/QDx1 | 95* | 221* | 13 | 88 | 100 |
| 3.194 | AB033-JL (E4) | 10/IP/QDx1 | 93* | 218* | 13 | 88 | 100 |
| 3.144 | AB033-SW | 10/IP/QDx1 | 92* | 184* | 0 | 88 | 88 |
| 3.143 | AB033-SX | 10/IP/QDx1 | 92* | 153* | 0 | 100 | 100 |
| 3.145 | AB033-TV | 10/IP/QDx1 | 84* | 108* | 0 | 63 | 63 |
| 3.146 | AB033-SZ | 10/IP/QDx1 | 85* | 168* | 0 | 63 | 63 |
| 3.149 | AB033-SY | 10/IP/QDx1 | 92* | 168* | 0 | 88 | 88 |
| 3.153 | AB033-TX | 10/IP/QDx1 | 93* | 161* | 0 | 100 | 100 |
| 3.195 | AB033-TX (E2) | 10/IP/QDx1 | 87* | 195* | 0 | 75 | 75 |
| 3.196 | AB033-TX (E4) | 10/IP/QDx1 | 84* | 147* | 0 | 75 | 75 |
| 3.157 | AB033-UK | 10/IP/QDx1 | 41* | 34* | 0 | 0 | 0 |
| 3.154 | AB033-TZ | 10/IP/QDx1 | 84* | 89* | 0 | 50 | 50 |
| 3.152 | AB033-TY | 10/IP/QDx1 | 73* | 47* | 0 | 13 | 13 |
| 3.151 | AB033-TR | 10/IP/QDx1 | 57* | 21 | 0 | 13 | 13 |
| 3.156 | AB033-UJ | 10/IP/QDx1 | 44 | 11 | 0 | 0 | 0 |
| 3.155 | AB033-UA | 10/IP/QDx1 | 57* | 29* | 0 | 0 | 0 |
| 3.161 | AB033-VB | 10/IP/QDx1 | 68* | 37* | 0 | 0 | 0 |

**IgG1 mAb
^{†}Non-targeting antibody
^{[a]}dose is given in mg/kg/day
*= p < 0.05 as compared to control treatment (AB095)

TABLE 12
Inhibition of H1650 xenograft tumor growth after treatment
with a single dose of EGFR-targeting Bcl-xLi ADC

| | | | Growth | | | | |
|---|---|---|---|---|---|---|---|
| | | | Inhibition | | Response Frequency | | |
| | | Dose^{[a]}/route/ | TGIₘₐₓ | TGD | CR | PR | OR |
| Ex. No. | Treatment | regimen | (%) | (%) | (%) | (%) | (%) |
| | AB095**^{, †} | 10/IP/QDx1 | 0 | 0 | 0 | 0 | 0 |
| 3.1 | AB033-CZ | 10/IP/QDx1 | 94* | 174* | 0 | 100 | 100 |
| 3.165 | AB033-VY | 10/IP/QDx1 | 93* | 163* | 13 | 75 | 88 |
| 3.166 | AB033-WI | 10/IP/QDx1 | 86* | 208* | 0 | 38 | 38 |
| 3.167 | AB033-WK | 10/IP/QDx1 | 81* | 97* | 0 | 13 | 13 |
| 3.162 | AB033-VC | 10/IP/QDx1 | 81* | 113* | 0 | 13 | 13 |
| 3.160 | AB033-UZ | 10/IP/QDx1 | 73* | 68* | 0 | 0 | 0 |
| 3.163 | AB033-VS | 10/IP/QDx1 | 65* | 74* | 0 | 0 | 0 |
| 3.164 | AB033-VT | 10/IP/QDx1 | 65* | 61* | 0 | 0 | 0 |

**IgG1 mAb
^{†}Non-targeting antibody
^{[a]}dose is given in mg/kg/day
*= p < 0.05 as compared to control treatment (AB095)

TABLE 13
Inhibition of H1650 xenograft tumor growth
after treatment with EGFR-targeting antibody,
AB033 and ‘non-targeting, ADC, MSL109-H

| | | Growth | | | | |
|---|---|---|---|---|---|---|
| | | Inhibition | | Response Frequency | | |
| | Dose^{[a]}/route/ | TGIₘₐₓ | TGD | CR | PR | OR |
| Treatment | regimen | (%) | (%) | (%) | (%) | (%) |
| AB033 | 3/IP/Q4Dx6 | 17* | 0 | 0 | 0 | 0 |
| AB033 | 3/IP/Q4Dx6 | 54* | 44* | 0 | 0 | 0 |
| AB033 | 10/IP/Q4Dx6 | 62* | 56* | 0 | 0 | 0 |
| MSL109^{†}-H | 3/IP/Q4Dx6 | 18* | 0 | 0 | 0 | 0 |
| MSL109^{†}-H | 10/IP/Q4Dx6 | 43* | 20* | 0 | 0 | 0 |
| MSL109^{†}-H | 10/IP/Q4Dx6 | 8 | 0 | 0 | 0 | 0 |
| MSL109^{†}-CZ | 3/IP/Q4Dx6 | 29* | 0 | 0 | 0 | 0 |
| MSL109^{†}-CZ | 3/IP/Q7Dx6 | 18* | 0 | 0 | 0 | 0 |
| MSL109^{†}-CZ | 10/IP/Q4Dx6 | 32* | 16 | 0 | 0 | 0 |
| MSL109^{†}-CZ | 3/IP/Q4Dx6 | 32* | 12 | 0 | 0 | 0 |

†Non-targeting antibody
^{[a]}dose is given in mg/kg/day
*= p < 0.05 as compared to control treatment (AB095)

### Example 9. EpCAM-Targeted ADCs Inhibit the Growth of Tumors In Vivo

### 9.1. Methods

Methods of cell culture, inoculation of tumor cells, tumor measurements and animal husbandry were as in Example 9. Treatment was initiated at 10 days (Table 14) post inoculation of tumor cells. The tumor size at onset of treatment was approximately 222 mm³. All conjugates and antibodies were given intraperitoneally. The doses and regimens of treatment are specified in Table 14. Each treatment group consisted of 8 mice.

### 9.2. Results

As discussed in Example 10, the efficacy of inhibition of H1650 xenografts growth with EpCAM-targeted ADCs is illustrated by Table 14, below. In the table, to refer to efficacy, the same parameters of amplitude and durability of response are used as in Example 9.

A Bcl-XLi conjugate of the EpCAM(ING-1) antibody against EpCAM inhibited tumor growth more effectively than a conjugate with the non-targeting antibody MSL109. The improved efficacy over the passive targeting control was evidenced for the CZ conjugates. The TGIₘₐₓ was increased between 1.3-fold while increase of TGD was 3 fold (Table 14).

TABLE 14
Inhibition of H1650 xenograft tumor growth after treatment with
α-EpCAM--targeting Bcl-xLi ADCs administered as single agent

| | | Growth inhibition | |
|---|---|---|---|
| Name treatment | Dose^{[a]}/route/regimen | TGIₘₐₓ (%) | TGD (%) |
| AB095 ** | 10/IP/Q4Dx6 | 0 | 0 |
| MSL109-CZ † | 10/IP/Q4Dx6 | 44* | 27* |
| α-EpCAM(ING-1)-CZ | 10/IP/Q4Dx6 | 58* | 81* |

** IgG1 mAb
† Non-targeting antibody
^{[a]}dose is given in mg/kg/day
*= p < 0.05 as compared to control treatment (AB09S)

### Example 10. Bcl-xLi Antibody-Drug Conjugates Mitigate Systemic Toxicity

### 10.1. Circumvention of Thrombocytopenia

Administration of Bcl-xLi ADCs as antibody drug conjugate can circumvent the systemic toxicity of the small molecule via selective targeting of the tumor. In this manner, the ADC can bypass systemic toxicity and allow tumor-specific efficacy via two possible mechanisms. First, for ADCs with a cell membrane permeating Bcl-xL inhibitor, the binding to the carrier antibody can limit systemic exposure to the small molecule. Second, the ADC can drive the internalization of a non-permeating Bcl-xL inhibitor and thus selectively affect tumor cells that carry the targeted antigen.

### 10.1.1. Method & Results

The influence of two Bcl-xL inhibitory ADCs on the number of circulating platelets in mice was tested following a single intraperitoneal injection (the inhibitory ADCs are comprised of anti-EGFR antibody AB033 and control synthons H and I (Examples 2.99 and 2.100) and are designated AB033-H and AB033-I). The anti-tetanus toxoid antibody AB095 was used as a negative control. Navitoclax (ABT-263, a dual Bcl-2 and Bcl-xL inhibitor), A-1331852 (a selective ccli permeable Bcl-xL inhibitor, Leverson et al, 2015, Sci. Transl Med. 7:279ra40.) and the unconjugated Bcl-xL inhibitor Example 1.13.7 caused thrombocytopenia which was maximal at 6 hours following injection of the compounds. A dose of 0.61 mg/kg, which is the equivalent amount of Bcl-xL inhibitor found in Bcl-xLi ADC at 30 mg/kg, decreased the platelet number 100-fold from a normal count of approximately 6*10⁵/mm³ to 6*10³/mm³.

In contrast, none of the Bcl-xL inhibitory ADCs caused a meaningful reduction of the platelets 6 hours after administration (Table 15) or at any time point during an observation period of 14 days. The latter observation renders induction of thrombocytopenia caused by slow release of the inhibitor from the ADCs is unlikely.

TABLE 15
Influence of Bcl-xLi ADCs with cell permeating Bcl-xL
inhibitors on the number of circulating platelets

| | | Lowest | Time to |
|---|---|---|---|
| | | thrombocyte | lowest count |
| Compound | Dose (mg/kg) | count | (hours) |
| none | | 594 | 0 |
| AB095 | 30 | 539 | 6 |
| ABT-263 | 100 | 10 | 6 |
| Example 1.13.7 | 0.61 | 6 | 6 |
| A-1331852 | 25 | 9 | 6 |
| AB033-I | 30 | 335 | 72 |
| AB033-I | 10 | 567 | 72 |
| AB033-H | 30 | 521 | 72 |

Platelet count is presented as 1/10³ of the platelet#/mm³

While various specific embodiments have been illustrated and described, it will be appreciated that various changes can be made without departing from the spirit and scope of the disclosure.

What is claimed is:

1. A Bcl-xL inhibitor according to structural formulae (IIa), (IIb), (IIc) or (IId), or a pharmaceutically acceptable salt thereof, 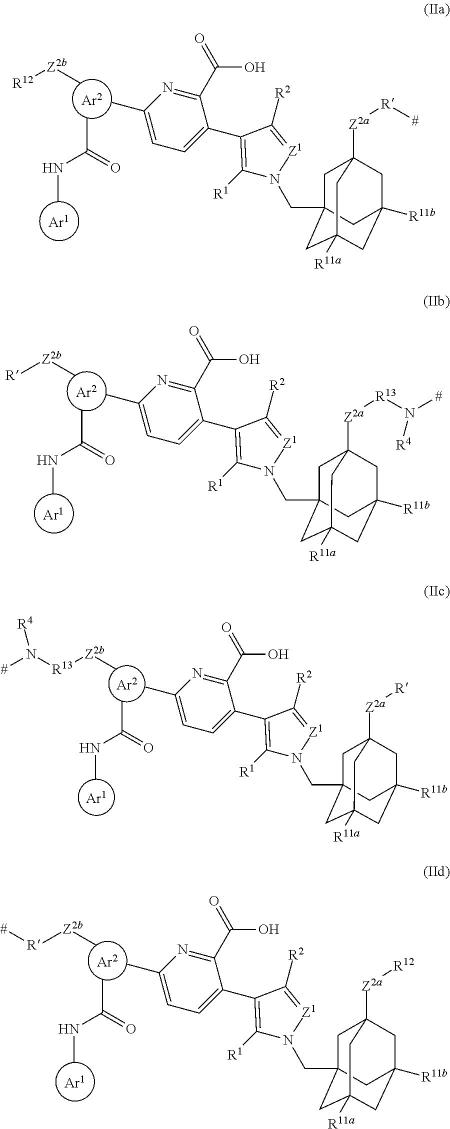
wherein:
Ar¹ is selected from 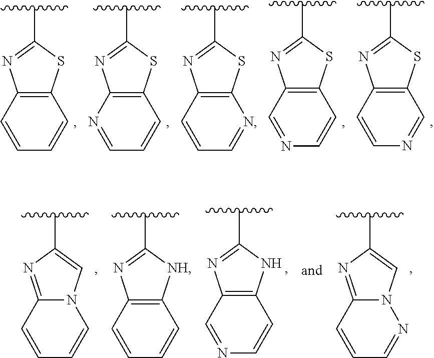
and is optionally substituted with one or more substituents independently selected from halo, hydroxy, nitro, lower alkyl, lower heteroalkyl, alkoxy, amino, cyano and halomethyl;
Ar² is selected from 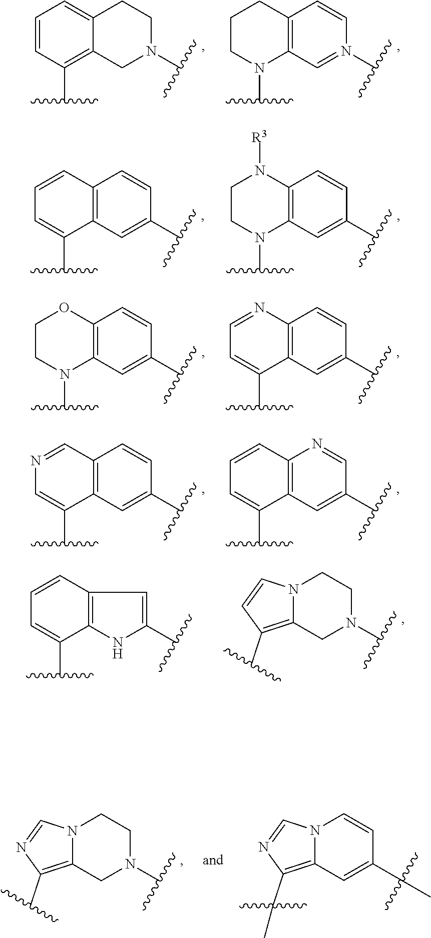
and is optionally substituted with one or more substituents independently selected from halo, hydroxy, nitro, lower alkyl, lower heteroalkyl, alkoxy, amino, cyano and halomethyl, wherein the R¹²—Z^{2b}—, R′—Z^{2b}—, #—N(R⁴)—R¹³—Z^{2b}—, or #—R′—Z^{2b}— substituents are attached to Ar² at any Ar² atom capable of being substituted;
Z¹ is selected from N, CH, C-halo, C—CH₃ and C—CN;
Z^{2a} and Z^{2b} are each, independently from one another, selected from a bond, NR⁶, CR^{6a} R^{6b}, O, S, S(O), SO₂, —NR⁶C(O)—, —NR^{6a}C(O)NR^{6b}—, and —NR⁶C(O)O—;
R′ is 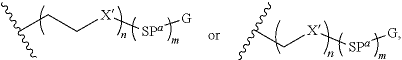
wherein #, where attached to R′, is attached to R′ at any R′ atom capable of being substituted;
X′ is selected at each occurrence from —N(R¹⁰)—, —N(R¹⁰)C(O)—, —N(R¹⁰)S(O)₂—, —S(O)₂N(R¹⁰)—, and —O—;
n is selected from 0-3;
R¹⁰ is independently selected at each occurrence from hydrogen, alkyl, heterocycle, aminoalkyl, G-alkyl, heterocycle, and —(CH₂)₂—O—(CH₂)₂—O—(CH₂)₂—NH₂;
G at each occurrence is independently selected from a polyol, a polyethylene glycol with between 4 and 30 repeating units, a salt and a moiety that is charged at physiological pH;
SP^{a} is independently selected at each occurrence from oxygen, S(O)₂N(H), N(H)S(O)₂, —N(H)C(O), C(O)N(H), N(H), arylene, heterocyclene, and optionally substituted methylene; wherein methylene is optionally substituted with one or more of —NH(CH₂)₂G, NH₂, alkyl, and carbonyl;
m is selected from 0-12;
R¹ is selected from hydrogen, methyl, halo, halomethyl, ethyl, and cyano;
R² is selected from hydrogen, methyl, halo, halomethyl and cyano;
R³ is selected from hydrogen, methyl, ethyl, halomethyl and haloethyl;
R⁴ is selected from hydrogen, lower alkyl and lower heteroalkyl or is taken together with an atom of R¹³ to form a cycloalkyl or heterocyclyl ring having between 3 and 7 ring atoms;
R⁶, R^{6a} and R^{6b} are each, independent from one another, selected from hydrogen, optionally substituted lower alkyl, optionally substituted lower heteroalkyl, optionally substituted cycloalkyl and optionally substituted heterocyclyl, or are taken together with an atom from R⁴ and at atom from R¹³ to form a cycloalkyl or heterocyclyl ring having between 3 and 7 ring atoms;
R^{11A} and R^{11b} are each, independently of one another, selected from hydrogen, halo, methyl, ethyl, halomethyl, hydroxyl, methoxy, CN, and SCH₃;
R¹² is optionally R′ or is selected from hydrogen, halo, cyano, optionally substituted alkyl, optionally substituted heteroalkyl, optionally substituted heterocyclyl, and optionally substituted cycloalkyl;
R¹³ is selected from optionally substituted alkylene, optionally substituted heteroalkylene, optionally substituted heterocyclene, and optionally substituted cycloalkylene; and
#represents either a hydrogen atom or the point of attachment to a linker L.

2.-101. (canceled)
